(19) United States
(12) Patent Application Publication
(10) Pub. No.: US 2005/0255114 A1
(43) Pub. Date: Nov. 17, 2005

(54) Methods and diagnosis for the treatment of preeclampsia

(75) Inventors: Ivan Labat, Mountain View, CA (US); Y. Tom Tang, San Jose, CA (US); Birgit Stache-Crain, Sunnyvale, CA (US); Bryan Boyle, Palo Cedro, CA (US)

Correspondence Address: NUVELO, INC, 675 ALMANOR AVE., SUNNYVALE, CA, 94085, US

(73) Assignee: NUVELO, Inc., Sunnyvale, CA (US)

(21) Appl. No.: 10/821,234

(22) Filed: Apr. 7, 2004

Related U.S. Application Data
(60) Provisional application No. 60/462,047, filed on Apr. 7, 2003.

(51) Int. Cl. C12Q 1/68; G01N 33/53; G01N 33/567; A61K 39/395

(52) U.S. Cl. 424/146.1; 435/6; 435/7.2

(57) ABSTRACT

Provided by the present invention are methods for treating and diagnosing preeclampsia, as well as kits for use in diagnosing patients with a higher risk of preeclampsia.

### 1. TECHNICAL FIELD

The present invention provides novel methods for the diagnosis of preeclampsia by determination of both lower and higher levels of proteins and/or polynucleotides or combinations thereof in patient tissue samples. Furthermore, it presents methods of treatment of preeclampsia through either inhibition of function of proteins or polynucleotides that are expressed higher in preeclampsia, or through replacement therapy of proteins or polynucleotides that are expressed lower in preeclampsia.

1.2 Sequence Listing

The sequences of the polynucleotides and polypeptides of the invention are listed in the Sequence Listing and are submitted on a compact disc containing the file labeled “821A.txt”-4.69 MB (4,921,344 bytes) which was created on an IBM PC, Windows 2000 operating system on Thursday, Apr. 1, 2004 at 12:22:06 PM. The Sequence Listing entitled “821A.txt” is herein incorporated by reference in its entirety. A computer readable format (“CRF”) and two duplicate copies (“Copy 1” and “Copy 2”) of the Sequence Listing “821A.txt” are submitted herein. Applicants hereby state that the content of the CRF and Copies 1 and 2 of the Sequence Listing, submitted in accordance with 37 C.F.R. §1.821(c) and (e), respectively, are the same.

### 2. BACKGROUND

Preeclampsia is a disorder complicating 5% of pregnancies in U.S. Moreover, it is the second major cause of maternal death in both U.S. and Japan, and is the leading cause of neonatal morbidity and mortality. It is a maternal disease, presenting with wide spectrum of symptoms, including hypertension, edema, proteinuria, fatigue, rapid weight gain, cardiac, pulmonary and renal failure. Pregnancy can further be complicated with hemolytic and liver manifestations, i.e. disease can progress into HELLP syndrome (Hemolysis, which is the breaking down of red blood cells, Elevated Liver enzymes, and Low Platelet count), or can progress into eclampsia, manifested by seizures and eventual death.

The fact that there is no effective predelivery treatment and that ultimately, the treatment of choice is delivery of the placenta, leads to two observations: First, there is a substantial lack of understanding of both etiology and pathophysiology of the disorder. Suggested treatments are symptomatic and include seizure prophylaxis with use of magnesium sulfate, aspirin (generally unsuccessful), and prevention by increasing the dietary intake of calcium. Attempts of mapping the genes or loci involved have yielded no obvious success, leaving the root cause of the disorder unknown. Secondly, research on the subject of preeclampsia has revolved around placental growth and angiogenesis, as both are central to the etiology of the disease. Available evidence implicates the placenta in the maternal systemic effects seen in preeclampsia. Therefore, extracellular placental factor(s) secreted by the placenta are likely to be synthesized by the placenta and may be useful in the diagnosis and therapy of preeclampsia.

Several differentially-expressed genes have been previously examined for roles in both diagnosis and treatment of preeclampsia. Despite this fact, there remains no reliable diagnostic nor treatment for this disease. Thus, there exists a need in the art to identify and develop agents, such as peptides, nucleic acids, or antibodies that provide therapeutic compositions and diagnostic methods for treating and identifying preeclampsia.

### 3. SUMMARY OF THE INVENTION

This invention is based on the discovery that SEQ ID NO: 1-852 encode nucleotides that are either up-regulated or down-regulated in preeclamptic placenta as compared to control placenta. SEQ ID NO: 853-1704 comprise the protein or protein fragments encoded by the nucleotide sequences of SEQ ID NO: 1-852.

The invention provides therapeutic and diagnostic methods for targeting tissue or tissue samples expressing SEQ ID NO: 853-1704 by using diagnostic, therapeutic, or targeting elements such as SEQ ID NO: 853-1704 polypeptides, and nucleic acids encoding said SEQ ID NO: 853-1704 polypeptides. SEQ ID NO: 1-852 exhibit differential expression profiles in preeclampic placental tissue samples as compared to healthy placental tissues (see Examples, specifically 3 and 11). Thus, detection of expression levels of these genes individually or in combination will provide a means through which to detect preeclampsia during pregnancy. Additionally, genes that exhibit lower expression in preeclamptic tissues as compared to healthy tissues provide a means through which a patient experiencing preeclampsia may be treated therapeutically, as replacement therapy with these gene products individually or in combination may ameliorate signs or symptoms of the disease. Furthermore, genes that exhibit higher expression in preeclamptic tissues as compared to healthy tissues provide a means through which a patient experiencing preeclampsia may be treated therapeutically, as targeting of these genes in order to decrease the effect of their overexpression may ameliorate signs or symptoms of the disease.

The present invention provides a variety of diagnostic elements and compositions. One such embodiment is a diagnostic kit comprising antibody preparation that is specific for SEQ ID NO: 853-1704. The kit would be utilized to detect levels of any of the polypeptides of SEQ ID NO: 853 through 1704 that are differentially expressed in tissues derived from preeclampic patients as compared to a normal range of expression derived from healthy patients. Exemplary diagnostic antibodies include a single antibody selective for a polypeptide of the invention, a combination of 2 or more such antibodies of the invention, and a combination of at least one such antibody of the invention with an antibody that does not recognize a polypeptide of the invention. Such a kit includes a standard for any of SEQ ID NO: 853-1704 indicative of a higher risk of diagnosis of preeclampsia. Exemplary tissues subject to this diagnostic kit include serum, plasma, urine, vaginal mucous, amniotic fluid, and fetal and/or maternal cells.

Another diagnostic embodiment of the invention is a kit comprising a protein chip for quantification of levels of SEQ ID NO: 853-1704 in biological samples. Such a chip is a solid surface array with specific chemical (e.g. anion, cation, hydrophobic, hydrophilic, metal) or biochemical (e.g. antibody, receptor, DNA, enzyme) moiety precoated on the surface. The chemical moieties are designed to capture and recognize whole classes of polypeptides from a tissue sample, while the biochemical moieties are designed to capture and recognize specific polypeptide sequences.

Another diagnostic embodiment of the invention is a kit comprising one or more polynucleotide sequences of SEQ ID NO: 1-852 coupled to a surface. Polynucleotides derived from the messenger RNA (mRNA) from fetal cells or maternal tissues will be hybridized to the surface-coupled polynucleotides of any of SEQ ID NO: 1-852. The kit will be utilized to detect differentially-expressed levels of one or more of the polynucleotides of SEQ ID NO: 1-852 in fetal cells or placental tissues derived from preeclamptic patients, as compared to a normal range of expression derived from healthy patients. Such a kit includes a standard for any of SEQ ID NO: 1-852 indicative of a higher risk of diagnosis of preeclampsia. An exemplary source of fetal cells or placental tissues is from cells or tissues shed into the maternal blood, and maternal cells as well.

The present invention provides for compositions that can be utilized for replacement therapeutics in order to ameliorate the signs and symptoms of preeclampsia wherein one or more of the genes set forth in SEQ ID NO: 1-852 are down-regulated in preeclampsia. One such embodiment is a composition comprising one or more isolated polypeptides that include, but are not limited to, a polypeptide comprising the polypeptide sequence set forth in SEQ ID NO: 853-1704; or a fragment of SEQ ID NO: 853-1704. This embodiment further includes amelioration of the signs or symptoms of preeclampsia via administration of one or more of the polypeptides of SEQ ID NO: 853-1704 and a pharmaceutically acceptable carrier.

Another embodiment of the invention is a composition comprising one or more isolated polynucleotides that include, but are not limited to, a polynucleotide comprising the polynucleotide sequence set forth in SEQ ID NO: 1-852; or a fragment of SEQ ID NO: 1-852; or a full length coding sequence SEQ ID NO: 1-852. This embodiment further includes amelioration of the signs or symptoms of preeclampsia via administration of one or more of the polynucleotides of SEQ ID NO: 1-852 and a pharmaceutically acceptable vector for delivery of a functional gene corresponding to polynucleotides of SEQ ID NO: 1-852, wherein the loss of normal function of the corresponding gene has been observed through diagnostics.

The present invention provides for targeting elements and compositions for genes expressed at higher levels in preeclamptic tissues than healthy. One such embodiment is a composition comprising an antibody preparation selective for a polypeptide of SEQ ID NO: 853-1704. Exemplary therapeutic antibodies include a single such antibody of the invention, a combination of two or more such antibodies of the invention, a combination of at least one antibody of the invention with an antibody that does not recognize a polypeptide of the invention, humanized antibodies that retain all or a portion of antigen-binding sites of said antibody or complementarity-determining regions (CDR) that recognize SEQ ID NO: 853-1704, Fab antibodies or fragments thereof, including a fragment of an antibody that retains one or more CDRs that recognize SEQ ID NO: 853-1704, and antibody fusion proteins that recognize a polypeptide of the invention.

Another targeting embodiment of the invention is a vaccine comprising a polypeptide of the invention, or a fragment or variant thereof and optionally comprising a suitable adjuvant.

### 4. BRIEF DESCRIPTION OF THE DRAWINGS

FIG. 1 shows the expression ratios of SEQ ID NO: 357 to SEQ ID NO: 551 relative to each other, using a real-time PCR protocol (TaqMan assay). The raw cycle count scores for each gene in the measured tissues were selected, with each score representing the number of doublings required to detect the message. The inverse expression ratios were computed for each gene in each measured tissue using the following formula:
(2 raised to the power of the SEQ ID NO: 347 raw cycle count score) divided by (2 raised to the power of the SEQ ID NO: 551 raw cycle count score).

### 5. DETAILED DESCRIPTION OF THE INVENTION

The development of the human fetus depends on the ability of trophoblast cells to invade the maternal tissues in order to anchor the placenta and fetus to the maternal endometrium and to gain access to, and control of, the maternal circulation. Human haemochorial placentation involves proliferation, migration and invasion of the endometrium and its vasculature by trophoblast cells (extravillous trophoblast, EVT). A completion of remodeling of uteroplacental arteries, which is dependent on trophoblast invasion, appears to provide a means of unhindered placental perfusion with maternal blood. Poor placental perfusion in a hypoinvasive placenta of preeclampsia is believed to impede fetal growth and thus affect fetal well being in some cases.

The pathophysiology of preeclampsia is poorly understood. Two stages of vascular dysfunction seem to be involved. In the early stage suboptimal development of the placenta and a hemodynamic maladaptation to pregnancy exist. At this stage maternal constitutional factors such as genetic and immunological factors and preexisting vascular diseases could play a role. However, no causal relationship has yet been proven. Extracellular placental factor(s), supposedly under the influence of ischemia due to defective placentation, may be responsible for the development of the preeclampsia condition rather than preeclampsia being a simple consequence of improper placentation. Thus, these factors may cause late vascular dysfunction characterized mainly by a generalized endothelial dysfunction, thus leading to the clinical presentation of preeclampsia.

There are three distinct regions of the maternal-fetal interface, any or all of which may be involved in the clinical manifestation of preeclampsia pregnancy disorder. It has been demonstrated that there is differential gene expression of some growth factors and their receptors between these regions in normal and preeclamptic pregnancy, and thus their spatial and temporal regulation is suggested in pregnancy disorders. The compositions of the present invention are based on an anlysis of tissues from the maternal-fetal interface including 2 different areas of the placental bed: the basal plate (deciduas basalis) and marginal zone (deciduas marginalis) as well as trophoblastic villi (see Examples 1-2).

The deciduas basalis is the portion of the maternal endometrium that participates with the chorion in the formation of the placenta at the site of implantation. The invasive function of the trophoblast cells is achieved by two distinctive pathways: 1) the villous pathway, in which cytotrophoblast cells proliferate and fuse, giving rise to the syncytiotrophoblast layer of the floating villi, which is engaged primarily in exchange and endocrine functions; and 2) the extravillous pathway, in which certain cytotrophoblast cells break out of the villi as discrete cell columns, migrate and invade the decidua and its vasculature. These “extravillous trophoblast cells” (EVT) are highly proliferative and invasive. In the placental bed, these cells segregate into several subsets. Of high importance are endovascular EVTs, which replace the endothelium (maternal blood vessel lining) of the uteroplacental arteries during the process of remodeling (endovascular invasion). Poor EVT cell invasion and remodeling of uteroplacental arteries are key features of preeclampsia, whereas uncontrolled invasion is a feature of trophoblastic neoplasias including choriocarcinomas.

The decidua marginalis represents the lateral part of the placenta, responsible for the lateral placental growth.

The trophoblast villi is from the middle area of placental body represent fetal placental pathology and could provide valuable information about placental development. Moreover, the analysis of molecular pathophysiology of this region could indicate abrogated functions in pregnancy disorders.

This invention is based on the discovery that SEQ ID NO: 1-852 encode nucleotides that are either up-regulated or down-regulated in preeclamptic placenta as compared to control placenta. SEQ ID NO: 853-1704 comprise the protein or protein fragments encoded by the nucleotide sequences of SEQ ID NO: 1-852.

5.1 Definitions

It must be noted that as used herein and in the appended claims, the singular forms “a”, “an” and “the” include plural references unless the context clearly dictates otherwise.

The term “active” refers to those forms of the polypeptide that retain the biologic and/or immunologic activities of any naturally occurring polypeptide. According to the invention, the terms “biologically active” or “biological activity” refer to a protein or peptide having structural, regulatory or biochemical functions of a naturally occurring molecule. Likewise “biologically active” or “biological activity” refers to the capability of the natural, recombinant or synthetic polypeptide of the invention, or any peptide thereof, to induce a specific biological response in appropriate animals or cells and to bind with specific antibodies.

The term “activated cells” as used in this application are those cells which are engaged in extracellular or intracellular membrane trafficking, including the export of secretory or enzymatic molecules as part of a normal or disease process.

The terms “complementary” or “complementarity” refer to the natural binding of polynucleotides by base pairing. For example, the sequence 5′-AGT-3′ binds to the complementary sequence 3′-TCA-5′. Complementarity between two single-stranded molecules may be “partial” such that only some of the nucleic acids bind or it may be “complete” such that total complementarity exists between the single stranded molecules. The degree of complementarity between the nucleic acid strands has significant effects on the efficiency and strength of the hybridization between the nucleic acid strands.

The term “embryonic stem cells (ES)” refers to a cell that can give rise to many differentiated cell types in an embryo or an adult, including the germ cells. The term “germ line stem cells (GSCs)” refers to stem cells derived from primordial stem cells that provide a steady and continuous source of germ cells for the production of gametes. The term “primordial germ cells (PGCs)” refers to a small population of cells set aside from other cell lineages particularly from the yolk sac, mesenteries, or gonadal ridges during embryogenesis that have the potential to differentiate into germ cells and other cells. PGCs are the source from which GSCs and ES cells are derived The PGCs, the GSCs and the ES cells are capable of self-renewal. Thus these cells not only populate the germ line and give rise to a plurality of terminally differentiated cells that comprise the adult specialized organs, but are able to regenerate themselves. The term “totipotent” refers to the capability of a cell to differentiate into all of the cell types of an adult organism. The term “pluripotent” refers to the capability of a cell to differentiate into a number of differentiated cell types that are present in an adult organism. A pluripotent cell is restricted in its differentiation capability in comparison to a totipotent cell.

The term “expression modulating fragment,” EMF, means a series of nucleotides that modulates the expression of an operably linked ORF or another EMF. As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs is nucleic acid fragments which induce the expression of an operably linked ORF in response to a specific regulatory factor or physiological event.

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences, A is adenine, C is cytosine, G is guanine, and T is thymine, while N is A, T, G, or C. It is contemplated that where the polynucleotide is RNA, the T (thymine) in the sequence herein may be replaced with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment”, “portion,” or “segment” or “probe” or “primer” are used interchangeably and refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention. Preferably the fragment comprises a sequence substantially similar to a portion of SEQ ID NO: 1-852.

Probes may, for example, be used to determine whether specific mRNA molecules are present in a cell or tissue or to isolate similar nucleic acid sequences from chromosomal DNA as described by Walsh et al. (Walsh, P. S. et al., PCR Methods Appl. 1:241-250 (1992)). They may be labeled by nick translation, Klenow fill-in reaction, PCR, or other methods well known in the art. Probes of the present invention, their preparation and/or labeling are elaborated in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y., both of which are incorporated herein by reference in their entirety.

The nucleic acid sequences of the present invention also include the sequence information from any of the nucleic acid sequences of SEQ ID NO: 1-852. The sequence information can be a segment of SEQ ID NO: 1-852 that uniquely identifies or represents the sequence information of SEQ ID NO: 1-852. One such segment can be a twenty-mer nucleic acid sequence because the probability that a twenty-mer is fully matched in the human genome is 1 in 300. In the human genome, there are three billion base pairs in one set of chromosomes. Because 4²⁰ possible twenty-mers exist, there are 300 times more twenty-mers than there are base pairs in a set of human chromosomes. Using the same analysis, the probability for a seventeen-mer to be fully matched in the human genome is approximately 1 in 5. When these segments are used in arrays for expression studies, fifteen-mer segments can be used. The probability that the fifteen-mer is fully matched in the expressed sequences is also approximately one in five because expressed sequences comprise less than approximately 5% of the entire genome sequence.

Similarly, when using sequence information for detecting a single mismatch, a segment can be a twenty-five mer. The probability that the twenty-five mer would appear in a human genome with a single mismatch is calculated by multiplying the probability for a full match (1÷4²⁵) times the increased probability for mismatch at each nucleotide position (3×25). The probability that an eighteen mer with a single mismatch can be detected in an array for expression studies is approximately one in five. The probability that a twenty-mer with a single mismatch can be detected in a human genome is approximately one in five.

The term “open reading frame,” ORF, means a series of nucleotide triplets coding for amino acids without any termination codons and is a sequence translatable into protein.

The terms “operably linked” or “operably associated” refer to functionally related nucleic acid sequences. For example, a promoter is operably associated or operably linked with a coding sequence if the promoter controls the transcription of the coding sequence. While operably linked nucleic acid sequences can be contiguous and in the same reading frame, certain genetic elements e.g. repressor genes are not contiguously linked to the coding sequence but still control transcription/translation of the coding sequence.

The term “pluripotent” refers to the capability of a cell to differentiate into a number of differentiated cell types that are present in an adult organism. A pluripotent cell is restricted in its differentiation capability in comparison to a totipotent cell.

The terms “polypeptide” or “peptide” or “amino acid sequence” refer to an oligopeptide, peptide, polypeptide, or protein sequence or fragment thereof and to naturally occurring or synthetic molecules. A polypeptide “fragment,” “portion,” or “segment” is a stretch of amino acid residues of at least about 5 amino acids, preferably at least about 7 amino acids, more preferably at least about 9 amino acids and most preferably at least about 17 or more amino acids. The peptide preferably is not greater than about 200 amino acids, more preferably less than 150 amino acids and most preferably less than 100 amino acids. Preferably the peptide is from about 5 to about 200 amino acids. To be active, any polypeptide must have sufficient length to display biological and/or immunological activity.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “translated protein coding portion” means a sequence which encodes for the full length protein which may include any leader sequence or a processing sequence.

The term “mature protein coding sequence” refers to a sequence which encodes a peptide or protein without any leader/signal sequence. The “mature protein portion” refers to that portion of the protein without the leader/signal sequence. The peptide may have the leader sequences removed during processing in the cell or the protein may have been produced synthetically or using a polynucleotide only encoding for the mature protein coding sequence. It is contemplated that the mature protein portion may or may not include an initial methionine residue. The initial methionine is often removed during processing of the peptide.

The term “derivative” refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as omithine, which do not normally occur in human proteins.

The term “variant” (or “analog”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other components normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term “recombinant expression system” means host cells which have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins that are transported across the membrane of the endoplasmic reticulum. “Secreted” proteins are also intended to include proteins containing non-typical signal sequences (e.g. Interleukin-1 Beta, see Krasney, P. A. and Young, P. R. Cytokine 4:134-143 (1992)) and factors released from damaged cells (e.g. Interleukin-1 Receptor Antagonist, see Arend, W. P. et. al. Annu. Rev. Immunol. 16:27-55 (1998)).

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.). Other exemplary hybridization conditions are described herein in the examples.

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

As used herein, “substantially equivalent” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 35% (i.e., the number of individual residue substitutions, additions, and/or deletions in a substantially equivalent sequence, as compared to the corresponding reference sequence, divided by the total number of residues in the substantially equivalent sequence is about 0.35 or less). Such a sequence is said to have 65% sequence identity to the listed sequence. In one embodiment, a substantially equivalent, e.g., mutant, sequence of the invention varies from a listed sequence by no more than 30% (70% sequence identity); in a variation of this embodiment, by no more than 25% (75% sequence identity); and in a further variation of this embodiment, by no more than 20% (80% sequence identity) and in a further variation of this embodiment, by no more than 10% (90% sequence identity) and in a further variation of this embodiment, by no more that 5% (95% sequence identity). Substantially equivalent, e.g., mutant, amino acid sequences according to the invention preferably have at least 80% sequence identity with a listed amino acid sequence, more preferably at least 90% sequence identity. Substantially equivalent nucleotide sequence of the invention can have lower percent sequence identities, taking into account, for example, the redundancy or degeneracy of the genetic code. Preferably, nucleotide sequence has at least about 65% identity, more preferably at least about 75% identity, and most preferably at least about 95% identity. For the purposes of the present invention, sequences having substantially equivalent biological activity and substantially equivalent expression characteristics are considered substantially equivalent. For the purposes of determining equivalence, truncation of the mature sequence (e.g., via a mutation which creates a spurious stop codon) should be disregarded. Sequence identity may be determined, e.g., using the Jotun Hein method (Hein, J. Methods Enzymol. 183:626-645 (1990)). Identity between sequences can also be determined by other methods known in the art, e.g. by varying hybridization conditions.

The term “totipotent” refers to the capability of a cell to differentiate into all of the cell types of an adult organism.

The term “transformation” means introducing DNA into a suitable host cell so that the DNA is replicable, either as an extrachromosomal element, or by chromosomal integration. The term “transfection” refers to the taking up of an expression vector by a suitable host cell, whether or not any coding sequences are in fact expressed. The term “infection” refers to the introduction of nucleic acids into a suitable host cell by use of a virus or viral vector.

As used herein, an “uptake modulating fragment,” UMF, means a series of nucleotides which mediate the uptake of a linked DNA fragment into a cell. UMFs can be readily identified using known UMFs as a target sequence or target motif with the computer-based systems described below. The presence and activity of a UMF can be confirmed by attaching the suspected UMF to a marker sequence. The resulting nucleic acid molecule is then incubated with an appropriate host under appropriate conditions and the uptake of the marker sequence is determined. As described above, a UMF will increase the frequency of uptake of a linked marker sequence.

Each of the above terms is meant to encompass all that is described for each, unless the context dictates otherwise.

5.2 Nucleic Acids of the Invention

The invention is based on the discovery of differentially-expressed polynucleotides in preeclamptic versus normal placental tissues, the polynucleotides encoding corresponding polypeptides and the use of these compositions for the diagnosis, treatment or prevention of preeclampsia.

The isolated polynucleotides of the invention include, but are not limited to a polynucleotide comprising any of the nucleotide sequences of SEQ ID NO: 1-852; a fragment of SEQ ID NO: 1-852; a polynucleotide comprising the full length protein coding sequence of SEQ ID NO: 1-852 (for example coding for SEQ ID NO: 853 through 1704); and a polynucleotide comprising the nucleotide sequence encoding the mature protein coding sequence of the polypeptides of any one of SEQ ID NO: 853-1704. The polynucleotides of the present invention also include, but are not limited to, a polynucleotide that hybridizes under stringent conditions to (a) the complement of any of the nucleotides sequences of SEQ ID NO: 1-852; (b) a polynucleotide encoding any one of the polypeptides of SEQ ID NO: 853-1704; (c) a polynucleotide which is an allelic variant of any polynucleotides recited above; (d) a polynucleotide which encodes a species homolog of any of the proteins recited above; or (e) a polynucleotide that encodes a polypeptide comprising a specific domain or truncation of the polypeptides of SEQ ID NO: 853-1704. Domains of interest may depend on the nature of the encoded polypeptide; e.g., domains in receptor-like polypeptides include ligand-binding, extracellular, transmembrane, or cytoplasmic domains, or combinations thereof; domains in immunoglobulin-like proteins include the variable immunoglobulin-like domains; domains in enzyme-like polypeptides include catalytic and substrate binding domains; and domains in ligand polypeptides include receptor-binding domains.

The polynucleotides of the invention include naturally occurring or wholly or partially synthetic DNA, e.g., cDNA and genomic DNA, and RNA, e.g., mRNA. The polynucleotides may include all of the coding region of the cDNA or may represent a portion of the coding region of the cDNA.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to any of the polynucleotides of SEQ ID NO: 1-852 can be obtained by screening appropriate cDNA or genomic DNA libraries under suitable hybridization conditions using any of the polynucleotides of SEQ ID NO: 1-852 or a portion thereof as a probe. Alternatively, the polynucleotides of SEQ ID NO: 1-852 may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri, and UniGene. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99% sequence identity to a polynucleotide recited above.

Included within the scope of the nucleic acid sequences of the invention are nucleic acid sequence fragments that hybridize under stringent conditions to any of the nucleotide sequences of SEQ ID NO: 1-852, or complements thereof, which fragment is greater than about 5 nucleotides, preferably 7 nucleotides, more preferably greater than 9 nucleotides and most preferably greater than 17 nucleotides. Fragments of, e.g. 15, 17, or 20 nucleotides or more that are selective for (i.e. specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate human genes from genes of other species, and are preferably based on unique nucleotide sequences.

The sequences falling within the scope of the present invention are not limited to these specific sequences, but also include allelic and species variations thereof. Allelic and species variations can be routinely determined by comparing the sequence provided in SEQ ID NO: 1-852, a representative fragment thereof, or a nucleotide sequence at least 90% identical, preferably 95% identical, to SEQ ID NO: 1-852 with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another codon that encodes the same amino acid is expressly contemplated.

The nearest neighbor result for the nucleic acids or polypeptides of the present invention, including SEQ ID NO: 1-852, and SEQ ID NO: 853-1704 can be obtained by searching a database using an algorithm or a program. Preferably, a BLAST which stands for Basic Local Alignment Search Tool is used to search for local sequence alignments (Altshul, S. F. J. Mol. Evol. 36 290-300 (1993) and Altschul S. F. et al. J. Mol. Biol. 21:403-410 (1990)).

Species homologs (or orthologs) of the disclosed polynucleotides and proteins are also provided by the present invention. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally occurring alternative forms of the isolated polynucleotide that also encode proteins which are identical, homologous or related to that encoded by the polynucleotides.

The nucleic acid sequences of the invention are further directed to sequences which encode variants of the described nucleic acids. These amino acid sequence variants may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence variants are preferably constructed by mutating the polynucleotide to encode an amino acid sequence that does not occur in nature. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells and sequences such as FLAG or poly-histidine sequences useful for purifying the expressed protein.

In a preferred method, polynucleotides encoding the novel amino acid sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant.

A further technique for generating amino acid variants is the cassette mutagenesis technique described in Wells et al., Gene 34:315 (1985); and other mutagenesis techniques well known in the art, such as, for example, the techniques in Sambrook, et al., Molecular Cloning: A Laboratory Manual, Cold Spring Harbor Laboratory, NY (1989), and Current Protocols in Molecular Biology, Ausubel et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those that are capable of hybridizing to the appropriate novel nucleic acid sequence under stringent conditions.

Polynucleotides encoding preferred polypeptide truncations of the invention can be used to generate polynucleotides encoding chimeric or fusion proteins comprising one or more domains of the invention and heterologous protein sequences.

The polynucleotides of the invention additionally include the complement of any of the polynucleotides recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions that can routinely isolate polynucleotides of the desired sequence identities.

In accordance with the invention, polynucleotide sequences comprising the mature protein coding sequences, coding for any one of SEQ ID NO: 853-1704, or functional equivalents thereof, may be used to generate recombinant DNA molecules that direct the expression of that nucleic acid, or a functional equivalent thereof, in appropriate host cells. Also included are the cDNA inserts of any of the clones identified herein.

A polynucleotide according to the invention can be joined to any of a variety of other nucleotide sequences by well-established recombinant DNA techniques (see Sambrook J et al. (1989) Molecular Cloning: A Laboratory Manual, Cold Spring Harbor Laboratory, NY). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having any of the nucleotide sequences of SEQ ID NO: 1-852 or a fragment thereof or any other polynucleotides of the invention. In one embodiment, the recombinant constructs of the present invention comprise a vector, such as a plasmid or viral vector, into which a nucleic acid having any of the nucleotide sequences of SEQ ID NO: 1-852 or a fragment thereof is inserted, in a forward or reverse orientation. In the case of a vector comprising one of the ORFs of the present invention, the vector may further comprise regulatory sequences, including for example, a promoter, operably linked to the ORF. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, and pSVL (Pharmacia/Pfizer).

The isolated polynucleotide of the invention may be operably linked to an expression control sequence such as the pMT2 or pED expression vectors disclosed in Kaufinan et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacd, lacZ, T3, T7, gpt, lambda PR, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers permitting transformation of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

Polynucleotides of the invention can also be used to induce immune responses. For example, as described in Fan et al., Nat. Biotech. 17:870-872 (1999), hereby incorporated herein by reference, nucleic acid sequences encoding a polypeptide may be used to generate antibodies against the encoded polypeptide following topical administration of naked plasmid DNA or following injection, and preferably intramuscular injection of the DNA. The nucleic acid sequences are preferably inserted in a recombinant expression vector and may be in the form of naked DNA.

Another aspect of the invention pertains to isolated antisense nucleic acid molecules that can hybridize to, or are complementary to, the nucleic acid molecules comprising SEQ ID NO: 1-852, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire coding strand of SEQ ID NO: 1-852, or to only a portion thereof. Nucleic acid molecules encoding fragments, homologs, derivatives and analogs of SEQ ID NO: 1-852 or antisense nucleic acids complementary to SEQ ID NO: 1-852 nucleic acid sequences of are additionally provided.

In one embodiment, an antisense nucleic acid molecule is antisense to a “coding region” of the coding strand of a nucleotide sequence encoding like protein of any of SEQ ID NO: 1-852. The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the proteins of SEQ ID NO: 853-1704. The term “conceding region” refers to 5′ and 3′ sequences which flank the coding region that are not translated into amino acids (i.e., also referred to as 5′ and 3′ untranslated regions).

Given the coding strand sequences encoding the polypeptides of SEQ ID NO: 853-1704 disclosed herein, antisense nucleic acids of the invention can be designed according to the rules of Watson and Crick or Hoogsteen base pairing. The antisense nucleic acid molecule can be complementary to the entire coding region of SEQ ID NO: 1-852-like mRNA, but more preferably is an oligonucleotide that is antisense to only a portion of the coding or noncoding region of said mRNA. For example, the antisense oligonucleotide can be complementary to the region surrounding the translation start site of SEQ ID NO: 1-852-like mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a polynucleotide or polypeptide of the invention to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they can be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave mRNA transcripts corresponding to SEQ ID NO: 1-852 to thereby inhibit translation of said mRNA. A ribozyme having specificity for SEQ ID NO: 1-852-like-encoding nucleic acid can be designed based upon the nucleotide sequence of a corresponding cDNA disclosed herein. For example, a derivative of a Tetrahymena L-19 IVS RNA can be constructed in which the nucleotide sequence of the active site is complementary to the nucleotide sequence to be cleaved in SEQ ID NO: 1-852-like encoding mRNA. See, e.g., U.S. Pat. No. 4,987,071 to Cech, et al. and U.S. Pat. No. 5,116,742 to Cech, et al. mRNA corresponding to SEQ ID NO: 1-852 can also be used to select a catalytic RNA having a specific ribonuclease activity from a pool of RNA molecules. See, e.g., Bartel et al., (1993) Science 261:1411-1418.

Alternatively, SEQ ID NO: 1-852 gene expression can be inhibited by targeting nucleotide sequences complementary to the regulatory region of the said nucleic acids (e.g., the promoter and/or enhancers of each gene corresponding to the polynucleotide of the invention) to form triple helical structures that prevent transcription of the genes in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the nucleic acids of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of the nucleic acids of the invention can be used in therapeutic and diagnostic applications. For example, PNAs can be used as antisense or antigene agents for sequence-specific modulation of gene expression by, e.g., inducing transcription or translation arrest or inhibiting replication. PNAs of the nucleic acids of the invention can also be used, for example, in the analysis of single base pair mutations in a gene (e.g., PNA directed PCR clamping; as artificial restriction enzymes when used in combination with other enzymes, e.g., S1 nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of nucleic acids of the invention can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of the nucleic acids of the invention can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. Supra, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide can be conjugated to another molecule, e.g., a peptide, a hybridization triggered cross-linking agent, a transport agent, a hybridization-triggered cleavage agent, and the like.

5.3 Use of Nucleic Acids as Probes

Another aspect of the subject invention is to provide for polypeptide-specific nucleic acid hybridization probes capable of hybridizing with naturally occurring nucleotide sequences. The hybridization probes of the subject invention may be derived from any of the nucleotide sequences SEQ ID NO: 1-852. Because the corresponding gene is only expressed in a limited number of tissues, a hybridization probe derived from of any of the nucleotide sequences SEQ ID NO: 1-852 can be used as an indicator of the presence of RNA of cell type of such a tissue in a sample.

Any suitable hybridization technique can be employed, such as, for example, in situ hybridization. PCR as described in U.S. Pat. Nos. 4,683,195 and 4,965,188 provides additional uses for oligonucleotides based upon the nucleotide sequences. Such probes used in PCR may be of recombinant origin, may be chemically synthesized, or a mixture of both. The probe will comprise a discrete nucleotide sequence for the detection of identical sequences or a degenerate pool of possible sequences for identification of closely related genomic sequences.

Other means for producing specific hybridization probes for nucleic acids include the cloning of nucleic acid sequences into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides. The nucleotide sequences may be used to construct hybridization probes for mapping their respective genomic sequences. The nucleotide sequence provided herein may be mapped to a chromosome or specific regions of a chromosome using well known genetic and/or chromosomal mapping techniques. These techniques include in situ hybridization, linkage analysis against known chromosomal markers, hybridization screening with libraries or flow-sorted chromosomal preparations specific to known chromosomes, and the like. The technique of fluorescent in situ hybridization of chromosome spreads has been described, among other places, in Verma et al (1988) Human Chromosomes: A Manual of Basic Techniques, Pergamon Press, New York N.Y.

Fluorescent in situ hybridization of chromosomal preparations and other physical chromosome mapping techniques may be correlated with additional genetic map data. Examples of genetic map data can be found in the 1994 Genome Issue of Science (265:1981f). Correlation between the location of a nucleic acid on a physical chromosomal map and a specific disease (or predisposition to a specific disease) may help delimit the region of DNA associated with that genetic disease. The nucleotide sequences of the subject invention may be used to detect differences in gene sequences between normal, carrier or affected individuals.

5.4 Preparation of Support Bound Oligonucleotides

Oligonucleotides, i.e., small nucleic acid segments, may be readily prepared by, for example, directly synthesizing the oligonucleotide by chemical means, as is commonly practiced using an automated oligonucleotide synthesizer.

Support bound oligonucleotides may be prepared by any of the methods known to those of skill in the art using any suitable support such as glass, polystyrene or Teflon. One strategy is to precisely spot oligonucleotides synthesized by standard synthesizers. Immobilization can be achieved using passive adsorption (Inouye & Hondo, 1990 J. Clin Microbiol 28(6) 1462-72); using UV light (Nagata et al., 1985; Dahlen et al., 1987; Morrissey & Collins, Mol. Cell Probes 1989 3(2) 189-207) or by covalent binding of base modified DNA (Keller et al., 1988; 1989); all references being specifically incorporated herein.

Another strategy that may be employed is the use of the strong biotin-streptavidin interaction as a linker. For example, Broude et al. (1994) Proc. Natl. Acad. Sci USA 91(8) 3072-6 describe the use of biotinylated probes, although these are duplex probes, that are immobilized on streptavidin-coated magnetic beads. Streptavidin-coated beads may be purchased from Dynal, Oslo. Of course, this same linking chemistry is applicable to coating any surface with streptavidin. Biotinylated probes may be purchased from various sources, such as, e.g., Operon Technologies (Alameda, Calif.).

Alternatively, DNA may be covalently bound to the microwell surface using Covalink NH technology (Nunc Laboratories, Naperville, Ill.). CovaLink NH is a polystyrene surface grafted with secondary amino groups (>NH) that serve as bridge-heads for further covalent coupling. CovaLink Modules may be purchased from Nunc Laboratories. DNA molecules may be bound to CovaLink exclusively at the 5′-end by a phosphoramidate bond, allowing immobilization of more than 1 pmol of DNA (Rasmussen et al., (1991) Anal Biochem 198(1) 138-42.

The use of CovaLink NH strips for covalent binding of DNA molecules at the 5′-end has been described (Rasmussen et al., 1991, Ann Biol Clin (Paris). 1990;48(9):647-50). In this technology, a phosphoramidate bond is employed (Chu et al., 1983 Nucleic Acids 11(18) 6513-29). This is beneficial as immobilization using only a single covalent bond is preferred. The phosphoramidate bond joins the DNA to the CovaLink NH secondary amino groups that are positioned at the end of spacer arms covalently grafted onto the polystyrene surface through a 2 nm long spacer arm. To link an oligonucleotide to Covaink NH via an phosphoramidate bond, the oligonucleotide terminus must have a 5′-end phosphate group. It is, perhaps, even possible for biotin to be covalently bound to CovaLink and then streptavidin used to bind the probes.

More specifically, the linkage method includes dissolving DNA in water (7.5 ng/ul) and denaturing for 10 min. at 95° C. and cooling on ice for 10 min. Ice-cold 0.1 M 1-methylimidazole, pH 7.0 (1-MeIm₇), is then added to a final concentration of 10 mM 1-MeIm₇. A single stranded DNA solution is then dispensed into CovaLink NH strips (75 ul/well) standing on ice.

Carbodiimide 0.2 M 1-ethyl-3-(3-dimethylaminopropyl)-carbodiimide (EDC), dissolved in 10 mM 1-MeIm₇, is made fresh and 25 ul added per well. The strips are incubated for 5 hours at 50° C. After incubation the strips are washed using, e.g., Nunc-Immuno Wash; first the wells are washed 3 times, then they are soaked with washing solution for 5 min., and finally they are washed 3 times (where in the washing solution is 0.4 N NaOH, 0.25% SDS heated to 50° C.).

It is contemplated that a further suitable method for use with the present invention is that described in PCT Patent Application WO 90/03382 (Southern & Maskos), hereby incorporated herein by reference. This method of preparing an oligonucleotide bound to a support involves attaching a nucleoside 3′-reagent through the phosphate group by a covalent phosphodiester link to aliphatic hydroxyl groups carried by the support. The oligonucleotide is then synthesized on the supported nucleoside and protecting groups removed from the synthetic oligonucleotide chain under standard conditions that do not cleave the oligonucleotide from the support. Suitable reagents include nucleoside phosphoramidite and nucleoside hydrogen phosphorate.

An on-chip strategy for the preparation of DNA probe for the preparation of DNA probe arrays may be employed. For example, addressable laser-activated photodeprotection may be employed in the chemical synthesis of oligonucleotides directly on a glass surface, as described by Fodor et al. (1991) Science 251(4995) 767-73, hereby incorporated herein by reference. Probes may also be immobilized on nylon supports as described by Van Ness et al. (1991) Nucleic Acids Res. 19(12) 3345-50; or linked to Teflon using the method of Duncan & Cavalier (1988) Anal Biochem 169(1) 104-8; all references being specifically incorporated herein.

To link an oligonucleotide to a nylon support, as described by Van Ness et al. (1991), requires activation of the nylon surface via alkylation and selective activation of the 5′-amine of oligonucleotides with cyanuric chloride.

One particular way to prepare support bound oligonucleotides is to utilize the light-generated synthesis described by Pease et al., (1994) Proc. Natl. Acad. Sci USA 91(11) 5022-6. These authors used current photolithographic techniques to generate arrays of immobilized oligonucleotide probes (DNA chips). These methods, in which light is used to direct the synthesis of oligonucleotide probes in high-density, miniaturized arrays, utilize photolabile 5′-protected N-acyl-deoxynucleoside phosphoramidites, surface linker chemistry and versatile combinatorial synthesis strategies. A matrix of 256 spatially defined oligonucleotide probes may be generated in this manner.

5.5 Preparation of Nucleic Acid Fragments

The nucleic acids may be obtained from any appropriate source, such as cDNAs, genomic DNA, chromosomal DNA, microdissected chromosome bands, cosmid or YAC inserts, and RNA, including mRNA without any amplification steps. For example, Sambrook et al. (1989) describes three protocols for the isolation of high molecular weight DNA from mammalian cells (p. 9.14-9.23).

DNA fragments may be prepared as clones in M13, plasmid or lambda vectors and/or prepared directly from genomic DNA or cDNA by PCR or other amplification methods. Samples may be prepared or dispensed in multiwell plates. About 100-1000 ng of DNA samples may be prepared in 2-500 ml of final volume.

The nucleic acids are then be fragmented by any of the methods known to those of skill in the art including, for example, using restriction enzymes as described at 9.24-9.28 of Sambrook et al. (1989), shearing by ultrasound and NaOH treatment.

Low pressure shearing is also appropriate, as described by Schriefer et al. (1990) Nucleic Acids Res. 18(24) 7455-6. In this method, DNA samples are passed through a small French pressure cell at a variety of low to intermediate pressures. A lever device allows controlled application of low to intermediate pressures to the cell. The results of these studies indicate that low-pressure shearing is a useful alternative to sonic and enzymatic DNA fragmentation methods.

One particularly suitable way for fragmenting DNA is contemplated to be that using the two base recognition endonuclease, CviJI, described by Fitzgerald et al. (1992) Nucleic Acids Res. 20(14) 3753-62. These authors described an approach for the rapid fragmentation and fractionation of DNA into particular sizes that they contemplated to be suitable for shotgun cloning and sequencing.

The restriction endonuclease CviJI normally cleaves the recognition sequence PuGCPy between the G and C to leave blunt ends. Atypical reaction conditions, which alter the specificity of this enzyme (CviJI**), yield a quasi-random distribution of DNA fragments form the small molecule pUC19 (2688 base pairs). Fitzgerald et al. (1992) quantitatively evaluated the randomness of this fragmentation strategy, using a CviJI** digest of pUC19 that was size fractionated by a rapid gel filtration method and directly ligated, without end repair, to a lac Z minus M13 cloning vector. Sequence analysis of 76 clones showed that CviJI** restricts pyGCPy and PuGCPu, in addition to PuGCPy sites, and that new sequence data is accumulated at a rate consistent with random fragmentation.

As reported in the literature, advantages of this approach compared to sonication and agarose gel fractionation include: smaller amounts of DNA are required (0.2-0.5 ug instead of 2-5 ug); and fewer steps are involved (no preligation, end repair, chemical extraction, or agarose gel electrophoresis and elution are needed).

Irrespective of the manner in which the nucleic acid fragments are obtained or prepared, it is important to denature the DNA to give single stranded pieces available for hybridization. This is achieved by incubating the DNA solution for 2-5 minutes at 80-90° C. The solution is then cooled quickly to 2° C. to prevent renaturation of the DNA fragments before they are contacted with the chip. Phosphate groups must also be removed from genomic DNA by methods known in the art.

5.6 Preparation of DNA Arrays

Arrays may be prepared by spotting DNA samples on a support such as a nylon membrane. Spotting may be performed by using arrays of metal pins (the positions of which correspond to an array of wells in a microtiter plate) to repeated by transfer of about 20 nl of a DNA solution to a nylon membrane. By offset printing, a density of dots higher than the density of the wells is achieved. One to 25 dots may be accommodated in 1 mm², depending on the type of label used. By avoiding spotting in some preselected number of rows and columns, separate subsets (subarrays) may be formed. Samples in one subarray may be the same genomic segment of DNA (or the same gene) from different individuals, or may be different, overlapped genomic clones. Each of the subarrays may represent replica spotting of the same samples. In one example, a selected gene segment may be amplified from 64 patients. For each patient, the amplified gene segment may be in one 96-well plate (all 96 wells containing the same sample). A plate for each of the 64 patients is prepared. By using a 96-pin device, all samples may be spotted on one 8×12 cm membrane. Subarrays may contain 64 samples, one from each patient. Where the 96 subarrays are identical, the dot span may be 1 mm² and there may be a 1 mm space between subarrays.

Another approach is to use membranes or plates (available from NUNC, Naperville, Ill.) which may be partitioned by physical spacers e.g. a plastic grid molded over the membrane, the grid being similar to the sort of membrane applied to the bottom of multiwell plates, or hydrophobic strips. A fixed physical spacer is not preferred for imaging by exposure to flat phosphor-storage screens or x-ray films.

5.7 Polypeptides of the Invention

The isolated polypeptides of the invention include, but are not limited to, a polypeptide comprising: the amino acid sequence set forth as any one of SEQ ID NO: 853-1704 or an amino acid sequence encoded by any one of the nucleotide sequences SEQ ID NO: 1-852 or the corresponding full length or mature protein. Polypeptides of the invention also include polypeptides preferably with biological or immunological activity that are encoded by: (a) a polynucleotide having any one of the nucleotide sequences set forth in SEQ ID NO: 1-852 or (b) polynucleotides encoding any one of the amino acid sequences set forth as SEQ ID NO: 853-1704 or (c) polynucleotides that hybridize to the complement of the polynucleotides of either (a) or (b) under stringent hybridization conditions. The invention also provides biologically active or immunologically active variants of any of the amino acid sequences set forth as SEQ ID NO: 853-1704 or the corresponding full length or mature protein; and “substantial equivalents” thereof (e.g., with at least about 65%, at least about 70%, at least about 75%, at least about 80%, 81%, 82%, 83%, 84%, 85%, 86%, 87%, 88%, or 89%, more typically at least about 90%, 91%, 92%, 93%, or 94% and even more typically at least about 95%, 96%, 97%, 98% or 99%, most typically at least about 99% amino acid identity) that retain biological activity. Polypeptides encoded by allelic variants may have a similar, increased, or decreased activity compared to polypeptides comprising SEQ ID NO: 853-1704.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114, 9245-9253 (1992), both of which are hereby incorporated herein by reference. Such fragments may be fused to carrier molecules such as immunoglobulins for many purposes, including increasing the valency of protein binding sites.

The present invention also provides both full-length and mature forms (for example, without a signal sequence or precursor sequence) of the disclosed proteins. The protein coding sequence is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form. Where proteins of the present invention are membrane bound, soluble forms of the proteins are also provided. In such forms, part or all of the regions causing the proteins to be membrane bound are deleted so that the proteins are fully secreted from the cell in which it is expressed.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

A variety of methodologies known in the art can be utilized to obtain any one of the isolated polypeptides or proteins of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptide. Thus, they may be employed as biologically active or immunological substitutes for natural, purified proteins in screening of therapeutic compounds and in immunological processes for the development of antibodies.

The polypeptides and proteins of the present invention can alternatively be purified from cells that have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell that produces one of the polypeptides or proteins of the present invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length or mature form of the protein.

In an alternative method, the polypeptide or protein is purified from bacterial cells that naturally produce the polypeptide or protein. One skilled in the art can readily follow known methods for isolating polypeptides and proteins in order to obtain one of the isolated polypeptides or proteins of the present invention. These include, but are not limited to, immunochromatography, High Performance Liquid Chromatography (HPLC), size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology. Polypeptide fragments that retain biological/immunological activity include fragments comprising greater than about 100 amino acids, or greater than about 200 amino acids, and fragments that encode specific protein domains.

The purified polypeptides can be used in in vitro binding assays that are well known in the art to identify molecules that bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

In addition, the peptides of the invention or molecules capable of binding to the peptides may be complexed with toxins, e.g., ricin or cholera, or with other compounds that are toxic to cells. The toxin-binding molecule complex is then targeted to a tumor or other cell by the specificity of the binding molecule for SEQ ID NO: 853-1704.

The protein of the invention may also be expressed as a product of transgenic animals, e.g., as a component of the milk of transgenic cows, goats, pigs, or sheep which are characterized by somatic or germ cells containing a nucleotide sequence encoding the protein.

The proteins provided herein also include proteins characterized by amino acid sequences similar to those of purified proteins but into which modification are naturally provided or deliberately engineered. For example, modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein. Regions of the protein that are important for the protein function can be determined by various methods known in the art including the alanine-scanning method which involved systematic substitution of single or strings of amino acids with alanine, followed by testing the resulting alanine-containing variant for biological activity. This type of analysis determines the importance of the substituted amino acid(s) in biological activity. Regions of the protein that are important for protein function may be determined by the eMATRIX program or the Pfam program.

Other fragments and derivatives of the sequences of proteins which would be expected to retain protein activity in whole or in part and are useful for screening or other immunological methodologies may also be easily made by those skilled in the art given the disclosures herein. Such modifications are encompassed by the present invention.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat™ kit), and such methods are well known in the art, as described in Summers and Smith, Texas Agricultural Experiment Station Bulletin No. 1555 (1987), hereby incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX), or as a His tag. Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“FLAG®”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

5.8 Determining Polypeptide and Polynucleotide Identity and Similarity

Preferred identity and/or similarity are designed to give the largest match between the sequences tested. Methods to determine identity and similarity are codified in computer programs including, but are not limited to, the GCG program package, including GAP (Devereux, J., et al., Nucleic Acids Research 12(1):387 (1984); Genetics Computer Group, University of Wisconsin, Madison, Wis.), BLASTP, BLASTN, BLASTX, FASTA (Altschul, S. F. et al., J. Molec. Biol. 215:403-410 (1990), PSI-BLAST (Altschul S. F. et al., Nucleic Acids Res. vol. 25, pp. 3389-3402, hereby incorporated herein by reference), the eMatrix software (Wu et al., J. Comp. Biol., vol. 6, pp. 219-235 (1999), hereby incorporated herein by reference), eMotif software (Nevill-Manning et al, ISMB-97, vol 4, pp. 202-209, hereby incorporated herein by reference), the GeneAtlas software Accelrys, Inc. San Diego, Calif.) (Sanchez and Sali (1998) Proc. Natl. Acad. Sci., 95, 13597-13602; Kitson D H et al, (2000) “Remote homology detection using structural modeling—an evaluation” Submitted; Fischer and Eisenberg (1996) Protein Sci. 5, 947-955), and the Kyte-Doolittle hydrophobocity prediction algorithm (J. Mol Biol, 157, pp. 105-31 (1982), hereby incorporated herein by reference). The BLAST programs are publicly available from the National Center for Biotechnology Information (NCBI) and other sources (BLAST Manual, Altschul, S., et al. NCB NLM NIH Bethesda, Md. 20894; Altschul, S., et al., J. Mol. Biol. 215:403-410 (1990).

5.9 Chimeric and Fusion Proteins

The invention also provides chimeric or fusion proteins of the polypeptides of the invention. As used herein “chimeric protein” of the polypeptides of the invention or “fusion protein” of the polypeptides of the invention comprises a polypeptide of the invention operatively linked to a polypeptide not of the invention. A “polypeptide not of the invention” refers to a polypeptide having an amino acid sequence corresponding to a protein that is not substantially homologous to the polypeptides of the invention, e.g., a protein that is different from the polypeptide or polypeptides of the invention in question and that is derived from the same or a different organism. Within a fusion protein of the polypeptides of the invention the polypeptide of the invention can correspond to all or a portion of a polypeptide of the invention. In one embodiment, a fusion protein of the polypeptide of the invention comprises at least one biologically active portion of a polypeptide of the invention. In another embodiment, a fusion protein of the polypeptide of the invention comprises at least two biologically active portions of a polypeptide of the invention. In yet another embodiment, a fusion protein of the polypeptide of the invention comprises at least three biologically active portions of a polypeptide of the invention. Within the fusion protein, the term “operatively-linked” is intended to indicate that the polypeptide of the invention and the polypeptide not of the invention are fused in-frame with one another. The polypeptide not of the invention can be fused to the N-terminus or C-terminus of polypeptide of the invention.

In one embodiment, the fusion protein is a GST-fusion protein of the polypeptides of the invention in which the polypeptide of the invention sequences are fused to the C-terminus of the GST (glutathione S-transferase) sequences. Such fusion proteins can facilitate the purification of recombinant polypeptides of the invention. In another embodiment, the fusion protein is a polypeptide of the invention containing a heterologous signal sequence at its N-terminus. In certain host cells (e.g., mammalian host cells), expression and/or secretion of the polypeptides of the invention can be increased through use of a heterologous signal sequence.

In yet another embodiment, the fusion protein is an immunoglobulin fusion protein of the polypeptide of the invention in which the sequences corresponding to the polypeptides of the invention are fused to sequences derived from a member of the immunoglobulin protein family. The immunoglobulin fusion proteins of the polypeptides of the invention can be incorporated into pharmaceutical compositions and administered to a subject to inhibit an interaction between ligand that is a polypeptide of the invention and a polypeptide of the invention on the surface of a cell, to thereby suppress signal transduction in vivo mediated by polypeptides of the invention. The immunoglobulin fusion proteins of the polypeptides of the invention can be used to affect the bioavailability of a polypeptide of the invention cognate ligand. Inhibition of the polypeptide of the invention ligand/polypeptide of the invention interaction can be useful therapeutically for both the treatment of proliferative and differentiative disorders, as well as modulating (e.g. promoting or inhibiting) cell survival. Moreover, the immunoglobulin fusion proteins of the polypeptides of the invention can be used as immunogens to produce anti-polypeptides of the invention-like antibodies in a subject, to purify ligands to the polypeptides of the invention, and in screening assays to identify molecules that inhibit the interaction of polypeptides of the invention with ligands for the polypeptide of the invention.

Chimeric or fusion protein of the polypeptides of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A nucleic acid encoding a polynucleotide or polypeptide of the invention can be cloned into such an expression vector such that the fusion moiety is linked in-frame to the polypeptide of the invention.

5.10 Hosts

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected by methods well-known to those of skill in the art, for example calcium phosphate transfection, DEAE, dextran mediated transfection, or electroporation (Davis, L. et al., Basic Methods in Molecular Biology (1986)). The host cells containing one of the polynucleotides of the invention, can be used in conventional manners to produce the gene product encoded by the isolated fragment (in the case of an ORF) or can be used to produce a heterologous protein under the control of the EMF.

Any host/vector system can be used to express one or more of the ORFs of the present invention. These include, but are not limited to, eukaryotic hosts such as HeLa cells, Cv-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated herein by reference.

Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell tines. Mammalian expression vectors will comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

5.11 Gene Therapy

Mutations in the polynucleotides of the invention gene may result in loss of normal function of the encoded protein. The invention thus provides gene therapy to restore normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp. 25-20 (1998). For additional reviews of gene therapy technology see Friedmann, Science, 244: 1275-1281 (1989); Verma, Scientific American: 68-84 (1990); and Miller, Nature, 357: 455-460 (1992). Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo in the presence of proteins of the present invention in order to proliferate or to produce a desired effect on or activity in such cells. Treated cells can then be introduced in vivo for therapeutic purposes. Alternatively, it is contemplated that in other human disease states, preventing the expression of or inhibiting the activity of polypeptides of the invention will be useful in treating the disease states. It is contemplated that antisense therapy or gene therapy could be applied to negatively regulate the expression of polypeptides of the invention.

Other methods inhibiting expression of a protein include the introduction of antisense molecules to the nucleic acids of the present invention, their complements, or their translated RNA sequences, by methods known in the art. Further, the polypeptides of the present invention can be inhibited by using targeted deletion methods, or the insertion of a negative regulatory element such as a silencer, which is tissue specific.

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of the polynucleotides of the present invention.

Knowledge of DNA sequences provided by the invention allows for modification of cells to permit, increase, or decrease, expression of endogenous polypeptide. Cells can be modified (e.g., by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequences include polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

5.12 Antibodies

Also included in the invention are antibodies to proteins, or fragments of proteins of the invention. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG, IgG₂, and others. Furthermore, in humans, the light chain may be a kappa chain or a lambda chain. Reference herein to antibodies includes a reference to all such classes, subclasses and types of human antibody species.

An isolated related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO: 853-1704, or designated in Tables from Examples 2, 4, and 5 and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a surface region of the protein, e.g., a hydrophilic region. A hydrophobicity analysis of the human related protein sequence will indicate which regions of a related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, Proc. Nat. Acad. Sci. USA 78: 3824-3828 (1981); Kyte and Doolittle, J. Mol. Biol. 157: 105-142 (1982), each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

The terms “specific for” and “selective for” indicate that the variable regions of the antibodies of the invention recognize and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the polypeptides of the invention are also contemplated, provided that the antibodies are first and foremost specific for, as defined above, full-length polypeptides of the invention. As with antibodies that are specific for full length polypeptides of the invention, antibodies of the invention that recognize fragments are those which can distinguish polypeptides from the same family of polypeptides despite inherent sequence identity, homology, or similarity found in the family of proteins.

Antibodies of the invention are useful for, for example, therapeutic purposes (by modulating activity of a polypeptide of the invention, or immunotargeting cells expressing said polypeptide of the invention to induce cell death or targeting by the immune system), diagnostic purposes to detect or quantitate a polypeptide of the invention, as well as purification of a polypeptide of the invention. Kits comprising an antibody of the invention for any of the purposes described herein are also comprehended. In general, a kit of the invention also includes a control antigen for which the antibody is immunospecific. The invention further provides a hybridoma that produces an antibody according to the invention. Antibodies of the invention are useful for detection and/or purification of the polypeptides of the invention.

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

The labeled antibodies of the present invention can be used for in vitro, in vivo, and in situ assays to identify cells or tissues in which a fragment of the polypeptide of interest is expressed. The antibodies may also be used directly in therapies or other diagnostics. The present invention further provides the above-described antibodies immobilized on a solid support. Examples of such solid supports include plastics such as polycarbonate, complex carbohydrates such as agarose and Sepharose®, acrylic resins and such as polyacrylamide and latex beads. Techniques for coupling antibodies to such solid supports are well known in the art (Weir, D. M. et al., “Handbook of Experimental Immunology” 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

5.12.1 Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

5.12.2 Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen-binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368:812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

5.12.3 Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann, et al., Nature, 332:323-327 (1988); Verhoeyen, et al., Science, 239:1534-1536 (1988)), by substituting rodent CDRs or CDR sequences for the corresponding sequences of a human antibody. (See also U.S. Pat. No. 5,225,539). In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechhmann et al., 1988; and Presta, Curr. Op. Struct. Biol., 2:593-596 (1992)).

5.12.4 Human Antibodies

Fully human antibodies relate to antibody molecules in which essentially the entire sequences of both the light chain and the heavy chain, including the CDRs, arise from human genes. Such antibodies are termed “human antibodies” or “fully human antibodies” herein. Human monoclonal antibodies can be prepared by the trioma technique; the human B-cell hybridoma technique (see Kozbor, et al., Immunol Today 4: 72 (1983)) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., Proc Natl Acad Sci USA 80: 2026-2030 (1983)) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10,:779-783 (1992)); Lonberg et al. (Nature 368:856-859 (1994)); Morrison (Nature 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

5.12.5 Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., Science 246:1275-1281 (1989)) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′) 2} fragment; (iii) an F_{ab} fragment generated by the treatment of the antibody molecule with papain and a reducing agent and (iv) Fᵥ fragments.

5.12.6 Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

5.12.7 Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

5.12.8 Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

5.12.9 Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, 131I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. In another embodiment, the antibody can be conjugated to a “receptor” (such streptavidin) for utilization in tumor pretargeting wherein the antibody-receptor conjugate is administered to the patient, followed by removal of unbound conjugate from the circulation using a claring agent and then administration of a “ligand” (e.g., avidin) that is in turn conjugated to a cytotoxic agent.

5.13 Assays for Detection of Polypeptides and Polynucleotides of the Invention

5.13.1 Sample Collection and Processing

The polypeptides of the invention individually or in combination are preferably quantified in a biological sample obtained from a patient. In the same manner, polynucleotides of the invention individually or in combination are prefereably quantified in a biological sample obtained from a patient. As used herein, a biological sample is a sample of biological tissue or fluid that contains a concentration of any of the polypeptides or polynucleotides of the invention that may be correlated with blood or tissue levels of any of the polypeptides or polynucleotides of the invention. Particularly preferred biological samples include blood serum (blood lacking a cellular component and clotting factors), blood plasma (blood lacking a cellular component), urine, cervicovaginal mucous, amniotic fluid, or fetal cells.

In one preferred embodiment, the polypeptides of the invention individually or in combination are quantified in whole blood or blood derivatives such as blood plasma or blood serum. Said samples are isolated from a patient according to standard methods well known to those of skill in the art, including venipuncture. After blood isolation from the patient, the blood sample may either be pretreated by dilution in an appropriate buffer solution, or be concentrated. Any of a number of standard aqueous buffer solutions at physiological pH may be employed, including phosphate, Tris, or others. Means of preparing blood serum or blood plasma are well known to those skilled in the art and typically involve centrifugation or filtration to produce blood plasma, or clotting followed by centrifugation or filtration to produce blood serum. Blood plasma or serum may be diluted by the addition of buffers or other reagents well known to those of skill in the art, and may be stored for up to 24 hours at 2-8° C., or at −20° C. or lower for longer periods, prior to measurement of any of the polypeptides of the invention individually or in combination.

In another preferred embodiment, any of the polynucleotides of the invention individually or in combination are quantified from cells present in the amniotic fluid. Said samples are isolated from the pregnant patient according to standard methods well known to those of skill in the art, including amniocentesis. Preferably within the second trimester of pregnancy, a thin, hollow needle is inserted from the abdomen through the uterus into the amniotic sac. A small amount (approximately 1 or 2 tablespoons) of amniotic fluid is withdrawn, with the needle then being removed. Subsequently, living cells from the withdrawn amniotic fluid are cultured for approximately two weeks before quantification of expression of any of the polynucleotides of the invention individually or in combination.

In another preferred embodiment, any of the polypeptides of the invention individually or in combination are quantified from vaginal mucous. Cervicovaginal mucous is removed from an area within the vaginal cavity such as the posterior formix, cervical canal, or uterine cavity. The sample is removed with a swab having a fibrous tip, aspirator, suction device, lavage device or the like and transferred to a suitable container for storage and transport to the testing laboratory. It is important that the sample be dispersed in a liquid that preserves the sensitive protein analytes such as any of the polypeptides of the invention which may be unstable in the sampled composition. The storage and transfer medium should prevent decline in the protein analyte level during storage and transfer. One example of a suitable preserving solution for storage and transfer consists of 0.05M Tris-HCL, pH 7.4; 0.15M NaCl, 0.02% NaN₃, 1% BSA, 500 Kallikrein Units/mL aprotinin, 1 mM phenylmethylsulfonyl fluoride (PMSF) and 5 mM EDTA.

In another preferred embodiment, any of the polypeptides of the invention individually or in combination are quantified from a sample of urine from a patient during pregnancy. The sample can be taken from any time of day, but preferably from the first morning void. One example of urine specimen collection and storage includes having the patient void into a plastic urine specimen container, and then immediately storing the container with the urine sample at 2 to 30° C. until testing. If the sample is to be stored for longer than 30 days, then it should be frozen between −20 and −70° C. until testing.

In another preferred embodiment, any of the polynucleotides of the invention individually or in combination are quantified from a sample of fetal cells and/or maternal cells derived from maternal blood. Fetal cell isolation from samples of maternal blood is achieved using any number of well-recognized detection and isolation techniques. (See for example Clin Chem Lab Med. 2002 February; 40(2):126-31, Int J Mol Med. 2002 September; 10(3):257-61, Cytometry. 2001 Dec. 1;45(4):267-76, and Expert Rev Mol Diagn. 2002 Jul.; 2(4):303-11 which are hereby incorporated by reference). Isolation of maternal blood cells is well-known to those of skill in the art.

5.13.2 Quantification of the Polypeptides of the Invention

Any of the polypeptides of the invention individually or in combination may be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, by surface-enhanced laser desorption/ionization (SELDI), and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, western blotting, and the like.

As used herein, an immunoassay is an assay that utilizes an antibody to specifically bind to the analyte. The immunoassay is characterized by the use of specific binding to a particular antibody as opposed to other physical or chemical properties to isolate, target, and quantify the analyte.

5.13.3 Immunological Binding Assays

In a preferred embodiment, any of the polypeptides of the invention individually or in combination may be detected and quantified using any of a number of well-recognized immunological binding assays. (See for example, U.S. Pat. No. 5,712,103 hereby incorporated herein by reference.) For a review of the general immunoassays, see also Methods in Cell Biology Volume 37: Antibodies in Cell Biology, Asai, ed. Academic Press, Inc. N.Y. (1993); Basic and Clinical Immunology 7^{th} Edition, Stites & Terr, eds. (1991), which are hereby incorporated herein by reference.

Immunological binding assays (or immunoassays) typically utilize a “capture agent” to specifically bind to and often immobilize the analyte (in this case any of the polypeptides of the invention). The capture agent is a moiety that specifically binds to the analyte. In a preferred embodiment, the capture agent is an antibody that specifically binds one of the polypeptides of the invention.

The antibody may be provided by any of a number of means well known to those of skill in the art (see, for example Methods in Cell Biology Vol. 37: Antibodies in Cell Biology, Asai, ed. Academic Press, Inc. N.Y. (1993); and Basic and Clinical Immunology 7^{th} Edition, Stites & Terr, eds. (1991), which are hereby incorporated herein by reference). The antibody may be a whole antibody or an antibody fragment. It may be polyclonal or monoclonal, and it may be produced by challenging an organism (e.g. mouse, rat, rabbit, etc.) with any of the polypeptides of the invention or an epitope derived therefrom. Alternatively, the antibody may be produced de novo using recombinant DNA methodology (e.g. DNA vaccination) and any of the polynucleotides of the invention in full-length gene form or a fragment derived therefrom. Additionally, antibodies that specifically bind any of the polypeptides of the invention may be produced using standard methods well-known to those of skill in the art, or may be obtained commercially.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled version of any of the polypeptides of the invention or a labeled antibody selectively recognizing any of the polypeptides of the invention. Alternatively, the labeling agent may be a third moiety, such as another antibody, that specifically binds to an antibody complexed with a polypeptide of the invention.

In a preferred embodiment, the labeling agent is an antibody that specifically binds to the capture agent (the antibody specifically recognizing any of the polypeptides of the invention). Such agents are well known to those of skill in the art, and most typically comprise labeled antibodies that specifically bind antibodies of the particular animal species from which the capture agent is derived. Thus, for example, where the capture agent is rabbit derived anti-human antibody to any of the polypeptides of the invention, the label agent may be a goat anti-rabbit IgG; an antibody that is specific to the constant region of the rabbit antibody.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species.

Throughout the assays, incubation and/or washing steps may be required after each combination of reagents. Incubation steps can vary from about 5 seconds to several hours, preferably from about 5 minutes to 24 hours. However, the incubation time will depend upon the assay format, analyte, volume of solution, concentration, and the like. Usually, the assays will be carried out at ambient temperature, although they can be conducted over a range of temperatures, such as 10° C. to 40° C.

5.13.4 Non-Competitive Assay Formats

Immunoassays for detecting any of the polypeptides of the invention may be either competitive or noncompetitive. Noncompetitive immunoassays are assays in which the amount of captured analyte (in this case any of the polypeptides of the invention) is directly measured. In one preferred “sandwich” assay, for example, the capture agent (antibodies selective for any of the polypeptides of the invention) can be bound directly to a solid substrate where they are immobilized. These immobilized antibodies then capture the protein encoded from any of the polypeptides of the invention present in the test sample. The protein thus immobilized is then bound by a labeling agent, such as a second human antibody selective for any of the polypeptides of the invention bearing a label. Alternatively, the second antibody may lack a label, but it may, in turn, be bound by a labeled third antibody specific to antibodies of the species from which the second antibody is derived.

Sandwich assays are particularly preferred as diagnostics for the present invention. Such an assay provides immobilized antibodies selective for any of the polypeptides of the invention, the polypeptides of the invention concentration standards, ¹²⁵I labeled antibodies selective for any of the polypeptides of the invention, and positive and negative controls for the polypeptides of the invention. As described above, the immobilized antibodies specifically bind to the the polypeptides of the invention present in the sample. Then the ¹²⁵I labeled antibodies selective for any of the polypeptides of the invention bind to the already bound polypeptides of the invention. Free ¹²⁵I labeled antibodies selective for any of the polypeptides of the invention are washed away and the remaining bound ¹²⁵I labeled antibodies selective for any of the polypeptides of the invention are detected using a gamma detector.

5.13.5 Competitive Assays

In competitive assays, the amount of analyte (any of the polypeptides of the invention) present in the sample is measured indirectly by measuring the amount of an added (exogenous) analyte (the polypeptides of the invention) displaced (or competed away) from a capture agent (antibody selective for the polypeptides of the invention) by the analyte present in the sample. In one competitive assay, a known amount of any of the polypeptides of the invention is added to the sample and the sample is then contacted with a capture agent, in this case an antibody that specifically binds to one of the polypeptides of the invention. The amount of the polypeptides of the invention bound to the antibody is inversely proportional to the concentration of protein present in the sample.

In a particularly preferred embodiment, the antibody is immobilized on a solid substrate. The amount of any of the polypeptides of the invention bound to the antibody may be determined either by measuring the amount of the polypeptides of the invention present in an complex consisting of an antibody specific for the polypeptides of the invention and the corresponding polypeptide of the invention, or alternatively by measuring the amount of remaining uncomplexed polypeptides of the invention. The amount of polypeptides of the invention may be detected by providing a labeled version of nay of the polypeptides of the invention.

A hapten inhibition assay is another preferred competitive assay. In this assay a known analyte, in this case any of the polypeptides of the invention is immobilized on a solid substrate. A known amount of antibody selective for any of the polypeptides of the invention is added to the sample, and the sample is then contacted with the immobilized quantity of the polypeptides of the invention. In this case, the amount of antibody selective for any of the polypeptides of the invention that is bound to the immobilized quantity of any of the polypeptides of the invention is proportional to the amount of actual polypeptides of the invention present in the sample. Again the amount of immobilized antibody may be detected by detecting either the immobilized fraction of antibody or the fraction of the antibody that remains in solution. Detection may be direct wherein the antibody is labeled or indirect by the subsequent addition of a labeled moiety that specifically binds to the antibody as described above.

5.13.6 Reduction of Non-Specific Binding

One of skill in the art will appreciate that it is often desirable to reduce non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this involves coating the substrate with a proteinaceous composition. In particular, protein compositions such as bovine serum albumin (BSA), nonfat powdered milk, and gelatin are widely used with powdered milk being most preferred.

5.13.7 Other Assay Formats

Western blot (immunoblot) analysis can also be used to detect and quantify the presence of any of the polypeptides of the invention in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with an antibody that specifically binds one of the polypeptides of the invention. These antibodies may be directly labeled or alternatively may be subsequently detected using labeled antibodies (e.g. labeled sheep anti-mouse antibodies) that specifically bind to the antibodies specific for the polypeptides of the invention.

5.13.8 Labels

The particular label or detectable group used in the assay is not a critical aspect of the invention, so long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads.™.), fluorescent dyes (e.g., fluorescein isothiocyanate, texas red, rhodamine, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g. polystyrene, polypropylene, latex, etc.) beads.

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is hereby incorporated herein by reference.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

5.13.9 Substrates

As mentioned above, depending upon the assay, various components, including the antigen, target antibody, or anti-human antibody, may be bound to a solid surface. Many methods for immobilizing biomolecules to a variety of solid surfaces are known in the art. For instance, the solid surface may be a membrane (e.g., nitrocellulose), a microtiter dish (e.g., PVC, polypropylene, or polystyrene), a test tube (glass or plastic), a dipstick (e.g. glass, PVC, polypropylene, polystyrene, latex, and the like), a microcentrifuge tube, or a glass or plastic bead. The desired component may be covalently bound or noncovalently attached through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials which may be employed, include paper, glasses, ceramics, metals, metalloids, semiconductive materials, cements or the like. In addition, are included substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

In preparing the surface, a plurality of different materials may be employed, particularly as laminates, to obtain various properties. For example, protein coatings, such as gelatin can be used to avoid non-specific binding, simplify covalent conjugation, enhance signal detection or the like.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. See, for example, Cuatrecasas, J. Biol. Chem. 245 3059 (1970) which is hereby incorporated herein by reference.

In addition to covalent bonding, various methods for noncovalently binding an assay component can be used. Noncovalent binding is typically nonspecific absorption of a compound to the surface. Typically, the surface is blocked with a second compound to prevent nonspecific binding of labeled assay components. Alternatively, the surface is designed such that it nonspecifically binds one component but does not significantly bind another. For example, a surface bearing a lectin such as Concanavalin A will bind a carbohydrate containing compound but not a labeled protein that lacks glycosylation. Various solid surfaces for use in noncovalent attachment of assay components are reviewed in U.S. Pat. Nos. 4,447,576 and 4,254,082, which are hereby incorporated herein by reference.

5.13.10 Mass Spectrometry

Levels of polypeptides can be detected also by mass spectrometry. Detection of disease biomarkers using mass spectrometry with protein biochip analysis is well known to those of skill in the art, and is detailed in: Rosty et al. Cancer Research 62: 1868 (2002), and Qu et al. Expert Rev Mol Diagn 2: 549 (2002), and are hereby incorporated herein by reference.

5.13.11 Determination of Levels of the Polypeptides of the Invention for Prediction of Preeclampsia

The preeclamptic condition is predicted by a statistically significant change in the concentration of any of the polypeptides of the invention individually or in combination in maternal blood, urine, or cervicovaginal mucous during pregnancy. Thus, the levels of any of the polypeptides of the invention individually or in combination provide an advantageous diagnostic of preeclampsia.

Baseline healthy levels of any of the polypeptides of the invention individually or in combination and levels indicative of preeclampsia may be determined by means well known to those of skill in the art. Generally this simply involves routine screening of healthy patients and patients that ultimately develop preeclampsia. Any variation between the two patient populations in the polypeptides of the invention individually or in combination that is determined by time of gestation, may be controlled by sampling uniformly at approximately the same gestational period, or alternatively, by determining blood, urine, or cervicovaginal mucous levels of the polypeptides of the invention (individually or in combination) as a function of period of gestation.

5.13.12 Quantification of the Polynucleotides of the Invention

Levels of any of the polynucleotides of the invention individually or in combination may be detected and quantified from tissue samples by any of a number of means well known to those of skill in the art. These may include analytic methods or techniques such as microarrays, PCR (polymerase chain reaction), RT-PCR (reverse transcriptase PCR), real-time PCR, TaqMan (Applied Biosystems), or Northern analysis.

5.14 Diagnostic Kits

5.14.1 Diagnostic Kits for Detection or Prediction of Preeclampsia Using any of the Polypeptides of the invention Individually or in Combination

The present invention provides for kits for the diagnosis of women at risk for preeclampsia. The kits preferably include one or more antibodies that specifically bind to the polypeptides of the invention. The antibodies may be free or immobilized on a solid support such as a test tube, a microtiter plate, a dipstick and the like. The kit may also contain instructional materials teaching the use of the antibody in an assay for the detection of a predilection to preeclampsia.

Additionally, the kit may contain one or more second antibodies that specifically bind to one of the polypeptides of the invention or alternatively, the first antibody. The second antibody may be labeled, or alternatively, the kit may contain a labeled third antibody that specifically binds the second antibody. The kit may also contain appropriate control series of any of the polypeptides of the invention, buffer solutions, positive and negative controls, washing solutions, dilution buffers and the like for the preparation and analysis of any of the polypeptides of the invention in samples derived from tissues, including blood plasma, blood serum, urine, or cervicovaginal mucous.

5.14.2 Diagnostic Kits for Detection or Prediction of Preeclampsia Using any of the Polynucleotides of the Invention Individually or in Combination

The present invention also provides for kits for the diagnosis of women at risk for preeclampsia. The kits preferably include one or more pairs of primers that specifically hybridize to the polynucleotides of the invention plus reagents well known to those of skill in the art for polymerase chain reaction (PCR) amplification of the polynucleotides of the invention, for example Medhurst et al. Methods Mol Med. 2003;79:229 and Dotsch et al. Expert Rev Mol Diagn. 2001 Jul.; 1(2):233.

Another preferred embodiment of the diagnostic kit for detection of preeclampsia using polynucleotides of the invention individually or in combination, is a microarray detection kit. Said microarray is comprised of a solid surface onto which single or double stranded polynucleotides of the invention are chemically or physically bonded or attached. Detection of levels of the polynucleotides of the invention are quantified by hybidization of the polynucleotides of the invention derived from tissue samples with like polynucleotides of the invention attached onto said solid surface. Either the polynucleotides of the invention derived from tissues or attached onto the solid surface may be attached to a detection label, for example Cy3 or Cy5 dyes (Amersham, Piscataway, N.J.). Microarray techniques are well known to those of skill in the art, and are detailed in Iyer V R, et al. (1999) Science 283(5398):83-7 and Whitney A R, et al. (2003). Proc Natl Acad Sci USA 100(4): 1896-901, hereby incorporated herein by reference.

5.15 Therapeutic Methods

The compositions (including polypeptide fragments, analogs, variants and antibodies or other binding partners or modulators including antisense polynucleotides) of the invention have applications in the treatment of preeclampsia.

5.15.1 Example

One embodiment of the invention is the administration of an effective amount of the one or more of the polypeptides or other composition of the invention to individuals affected by preeclampsia that can be modulated by regulating the peptides of the invention. While the mode of administration is not particularly important, parenteral administration is preferred. An exemplary mode of administration is to deliver an intravenous bolus. The dosage of polypeptides of the invention or other composition of the invention will normally be determined by the prescribing physician. It is to be expected that the dosage will vary according to the age, weight, condition and response of the individual patient. Typically, the amount of polypeptide administered per dose will be in the range of about 0.01 μg/kg to 100 mg/kg of body weight, with the preferred dose being about 0.1 μg/kg to 10 mg/kg of patient body weight. For parenteral administration, polypeptides of the invention or other composition of the invention will be formulated in an injectable form combined with a pharmaceutically acceptable parenteral vehicle. Such vehicles are well known in the art and examples include water, saline, Ringer's solution, dextrose solution, and solutions consisting of small amounts of the human serum albumin. The vehicle may contain minor amounts of additives that maintain the isotonicity and stability of the polypeptide or other active ingredient. The preparation of such solutions is within the skill of the art.

5.16 Pharmaceutical Formulations and Routes of Administration

A polypeptide or other composition of the present invention (from whatever source derived, including without limitation from recombinant and non-recombinant sources and including antibodies and other binding partners of the polypeptides of the invention) may be administered to a patient in need, by itself, or in pharmaceutical compositions where it is mixed with suitable carriers or excipient(s) at doses to treat or ameliorate a variety of disorders. Such a composition may optionally contain (in addition to protein or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. In further compositions, proteins of the invention may be combined with other agents beneficial to the treatment of the disease or disorder in question. These agents include various growth factors such as epidermal growth factor (EGF), platelet-derived growth factor (PDGF), transforming growth factors (TGF-α and TGF-β), insulin-like growth factor (IGF), as well as cytokines described herein.

The pharmaceutical composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. Conversely, protein or other active ingredient of the present invention may be included in formulations of the particular clotting factor, cytokine, lymphokine, other hematopoietic factor, thrombolytic or anti-thrombotic factor, or anti-inflammatory agent to minimize side effects of the clotting factor, cytokine, lymphokine, other hematopoietic factor, thrombolytic or anti-thrombotic factor, or anti-inflammatory agent (such as IL-IRa, IL-1 Hy1, IL-1 Hy2, anti-TNF, corticosteroids, immunosuppressive agents). A protein of the present invention may be active in multimers (e.g., heterodimers or homodimers) or complexes with itself or other proteins. As a result, pharmaceutical compositions of the invention may comprise a protein of the invention in such multimeric or complexed form.

As an alternative to being included in a pharmaceutical composition of the invention including a first protein, a second protein or a therapeutic agent may be concurrently administered with the first protein (e.g., at the same time, or at differing times provided that therapeutic concentrations of the combination of agents is achieved at the treatment site). Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein or other active ingredient of the present invention is administered to a mammal having a condition to be treated. Protein or other active ingredient of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein or other active ingredient of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein or other active ingredient of the present invention in combination with cytokine(s), lymphokine(s), other hematopoietic factor(s), thrombolytic or anti-thrombotic factors.

5.16.1 Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein or other active ingredient of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

Alternately, one may administer the compound in a local rather than systemic manner, for example, via injection of the compound directly into the amniotic cavity or placental tissue, often in a depot or sustained release formulation. Furthermore, one may administer the drug in a targeted drug delivery system, for example, in a liposome coated with a specific antibody, targeting, for example, placental tissue. The liposomes will be targeted to and taken up selectively by the afflicted tissue.

The polypeptides of the invention are administered by any route that delivers an effective dosage to the desired site of action. The determination of a suitable route of administration and an effective dosage for a particular indication is within the level of skill in the art. Suitable dosage ranges for the polypeptides of the invention can be extrapolated from these dosages or from similar studies in appropriate animal models. Dosages can then be adjusted as necessary by the clinician to provide maximal therapeutic benefit.

5.16.2 Compositions and Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein or other active ingredient of the present invention, and preferably from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein or other active ingredient of the present invention, and preferably from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. A preferred pharmaceutical composition for intravenous, cutaneous, or subcutaneous injection should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with pharmaceutically compatible counter ions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by NMC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are hereby incorporated herein by reference.

The amount of protein or other active ingredient of the present invention in the pharmaceutical composition of the present invention will depend upon the nature and severity of the condition being treated, and on the nature of prior treatments which the patient has undergone. Ultimately, the attending physician will decide the amount of protein or other active ingredient of the present invention with which to treat each individual patient. Initially, the attending physician will administer low doses of protein or other active ingredient of the present invention and observe the patient's response. Larger doses of protein or other active ingredient of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein or other active ingredient of the present invention per kg body weight. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Therapeutically useful agents other than a protein or other active ingredient of the invention which may also optionally be included in the composition as described above, may alternatively or additionally, be administered simultaneously or sequentially with the composition in the methods of the invention.

The therapeutic compositions are also presently valuable for veterinary applications. Particularly domestic animals and thoroughbred horses, in addition to humans, are desired patients for such treatment with proteins or other active ingredient of the present invention. The dosage regimen of a protein-containing pharmaceutical composition to be used will be determined by the attending physician considering various factors which modify the action of the proteins, e.g., the patient's age, sex, and diet, the severity of any infection, time of administration and other clinical factors. The dosage may vary with the type protein used in co-administration. For example, the addition of other known protein factors, cytokines or the like to the final composition, may also affect the dosage. Progress can be monitored by periodic assessment of symptoms (for example high blood pressure).

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

5.16.3 Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated (for example, high blood pressure). Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound which achieves a half-maximal inhibition of the protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p.1. Dosage amount and interval may be adjusted individually to provide plasma levels of the active moiety which are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for polypeptides or other compositions of the invention will be in the range of about 0.01 μg/kg to 100 mg/kg of body weight daily, with the preferred dose being about 0.1 μg/kg to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

5.16.4 Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

5.17 Examples

The present invention is illustrated in the following examples. Upon consideration of the present disclosure, one of skill in the art will appreciate that many other embodiments and variations may be made in the scope of the present invention. Accordingly, it is intended that the broader aspects of the present invention not be limited to the disclosure of the following examples. The present invention is not to be limited in scope by the exemplified embodiments which are intended as illustrations of single aspects of the invention, and compositions and methods which are functionally equivalent are within the scope of the invention. Indeed, numerous modifications and variations in the practice of the invention are expected to occur to those skilled in the art upon consideration of the present preferred embodiments. Consequently, the only limitations which should be placed upon the scope of the invention are those which appear in the appended claims.

All references cited within the body of the instant specification are hereby incorporated by reference in their entirety.

### EXAMPLES

### Example 1

### Isolation of SEQ ID NO: 1 through 852 from a cDNA Libraries of Human Cells

The novel nucleic acids of SEQ ID NO: 1 through 852 were obtained from several human normal and pre-eclamptic placental cDNA libraries using standard PCR, sequencing by hybridization sequence signature analysis, and Sanger sequencing techniques. The inserts of the library were amplified with PCR using primers specific for vector sequences flanking the inserts. These samples were spotted onto nylon membranes and interrogated with oligonucleotide probes to give sequence signatures. The clones were clustered into groups of similar or identical sequences, and single representative clones were selected from each group for gel sequencing. The 5′ sequence of the amplified inserts were then deduced using the reverse M13 sequencing primer in a typical Sanger sequencing protocol. PCR products were purified and subjected to fluorescent dye terminator cycle sequencing. Single-pass gel sequencing was done using a 377 Applied Biosystems (ABI) sequencer. These inserts was identified as a novel sequence not previously obtained from this library and not previously reported in public databases. These sequences are designated as SEQ ID NO: 1 through 852 in the attached sequence listing.

### Example 2

### Assemblage OF SEQ ID NO: 1 Through 852

The nucleic acids (SEQ ID NO: 1 through 852) of the invention were assembled from sequences that were obtained from various cDNA libraries by methods described in Example 1 above, and in some cases obtained from one or more public databases. The final sequence was assembled using the EST sequence as seed. Then a recursive algorithm was used to extend the seed into an extended assemblage, by pulling additional sequences from different databases (i.e. Nuvelo's database containing EST sequences, dbEST, gb pri, and UniGene) that belong to this assemblage. The algorithm terminated when there was no additional sequences from the above databases that would extend the assemblage. Inclusion of component sequences into the assemblage was based on a BLASTN hit to the extending assemblage with BLAST score greater than 300 and percent identity greater than 95%.

The nearest neighbor results for the assembled contigs (SEQ ID NO: 1-852) were obtained by using the BLASTN algorithm against the nucleotide (nt) database from Genbank (National Center for Biotechnology Information http://www.ncbi.nlm.nih.gov). The nucleotide database used was the version as of Dec. 30, 2002. The nearest neighbor results for the polypeptides (SEQ ID NO: 853-1704) corresponding to the assembled contigswere obtained by a FASTA search against Genpept, using FASTXY algorithm. FASTXY is an improved version of FASTA alignment that allows in-codon frame shifts. The version of the Genpept database was the version as of Jan. 10, 2003. The nearest neighbor results showed the closest homologue for each assemblage from Genpept (and contain the translated amino acid sequences for which the assemblages encodes SEQ ID NO 853 through 1704). The predicted amino acid sequences corresponding to SEQ ID NO: 1 through 852 were obtained by using a software program called FASTY (available from http://fasta:bioch.virginia.edu) which selects a polypeptide based on a comparison of translated novel polynucleotide to known polynucleotides (W. R. Pearson, Methods in Enzymology, 183:63-98 (1990), incorporated herein by reference). For ease of creating SEQ ID Numbers, the following formula was used: nucleotide SEQ ID NO added to 852. (For Example, the polypeptide corresponding to nucleotide SEQ ID NO 1 is SEQ ID NO 853).

The nearest neighbor results for both the nucleotide contigs (SEQ ID NO 1 through 852) and corresponding polypeptides (SEQ ID NO 853 through 1704) are set forth below. In some cases, no nearest neighbor result could be determined, and therefore are excluded from the following table.

TABLE 1

| SEQ | Genbank | | | | |
|---|---|---|---|---|---|
| ID | Accesssion | BLAST | Percent | | |
| NO: | Number | Score | Identity | Annotation | Organism |
| 1 | AF090949 | 1710 | 97 | HBV-X associated protein 2 | Cercopithecus |
| | | | | | aethiops |
| 1 | U31913 | 1736 | 99 | HBV-X associated protein | Homo sapiens |
| 1 | U78521 | 1740 | 100 | immunophilin homolog ARA9 | Homo sapiens |
| 2 | AAH12333 | 1168 | 100 | BC012333 Unknown (protein for | Homo sapiens |
| | | | | MGC: 2084) | |
| 2 | AF187981_1 | 1168 | 100 | AF187981 delta7-sterol-C5- | Homo sapiens |
| | | | | desaturase | |
| 2 | D85181 | 1273 | 100 | fungal sterol-C5-desaturase | Homo sapiens |
| | | | | homolog | |
| 4 | A84511 | 1017 | 99 | unnamed protein product | unidentified |
| 4 | AF498970_1 | 1016 | 100 | AF498970 small GTP binding | Homo sapiens |
| | | | | protein RhoA | |
| 4 | AX427410 | 1017 | 99 | unnamed protein product | unidentified |
| 5 | AAH04312 | 485 | 98 | BC004312 insulin-like growth | Homo sapiens |
| | | | | factor binding protein 2 (36 kD) | |
| 5 | AAH09902 | 485 | 98 | BC009902 Unknown (protein for | Homo sapiens |
| | | | | MGC: 2339) | |
| 5 | AAH12769 | 485 | 98 | BC012769 Unknown (protein for | Homo sapiens |
| | | | | MGC: 16256) | |
| 6 | AAH02434 | 430 | 100 | BC002434 p8 protein (candidate of | Homo sapiens |
| | | | | metastasis 1) | |
| 6 | AF069074 | 430 | 100 | P8 protein | Homo sapiens |
| 6 | AF135266_1 | 430 | 100 | AF135266 p8 protein homolog | Homo sapiens |
| 7 | AB047892 | 258 | 82 | hypothetical protein | Macaca |
| | | | | | fascicularis |
| 8 | AAH01199 | 833 | 99 | BC001199 Similar to glucose | Homo sapiens |
| | | | | regulated protein, 58 kDa | |
| 8 | AJ440721 | 833 | 99 | thioredoxin related protein | Homo sapiens |
| 8 | AL834423 | 833 | 99 | hypothetical protein | Homo sapiens |
| 9 | AAH00866 | 1124 | 100 | BC000866 tissue inhibitor of | Homo sapiens |
| | | | | metalloproteinase 1 (erythroid | |
| | | | | potentiating activity, | |
| | | | | collagenase inhibitor) | |
| 9 | M59906 | 1124 | 100 | collagenase inhibitor | Homo sapiens |
| 9 | X03124 | 1124 | 100 | precursor | Homo sapiens |
| 10 | AAH00518 | 834 | 83 | BC000518 Similar to brain acid- | Homo sapiens |
| | | | | soluble protein 1 | |
| 10 | AF039656 | 822 | 81 | neuronal tissue-enriched acidic | Homo sapiens |
| | | | | protein | |
| 10 | AF521668_1 | 516 | 59 | AF521668 CAP23 | Gallus |
| | | | | | gallus |
| 11 | AAH05931 | 733 | 100 | BC005931 hemoglobin, alpha 2 | Homo sapiens |
| 11 | AAH08572 | 733 | 100 | BC008572 hemoglobin, alpha 2 | Homo sapiens |
| 11 | BC032122 | 733 | 100 | hemoglobin, alpha 2 | Homo sapiens |
| 12 | AF347006 | 1171 | 90 | cytochrome c oxidase subunit I | Homo sapiens |
| 12 | AF382012 | 1159 | 91 | cytochrome c oxidase subunit I | Homo sapiens |
| 12 | AF382013 | 1159 | 91 | cytochrome c oxidase subunit I | Homo sapiens |
| 13 | AF242729_1 | 1242 | 100 | AF242729 HT022 | Homo sapiens |
| 13 | AJ245874 | 1242 | 100 | putative ATG/GTP binding protein | Homo sapiens |
| 13 | AJ250344 | 1242 | 100 | hypothetical protein | Homo sapiens |
| 14 | AAH17050 | 1367 | 100 | BC017050 2,3-bisphosphoglycerate | Homo sapiens |
| | | | | mutase | |
| 14 | M23068 | 1367 | 100 | 2,3-bisphosphoglycerate | Homo sapiens |
| 14 | X04327 | 1367 | 100 | 2,3 biphosphoglycerated mutase | Homo sapiens |
| | | | | (AA 1-259) | |
| 15 | AAH02334 | 921 | 100 | BC002334 DAZ associated protein 2 | Homo sapiens |
| 15 | AAH07900 | 921 | 100 | BC007900 DAZ associated protein 2 | Homo sapiens |
| 15 | D31767 | 921 | 100 | KIAA0058 | Homo sapiens |
| 16 | AAH09571 | 308 | 98 | BC009571 Similar to stimulated by | Homo sapiens |
| | | | | retinoic acid 13 | |
| 16 | U95006 | 309 | 98 | D9 splice variant A | Homo sapiens |
| 16 | U95007 | 273 | 98 | D9 splice variant B | Homo sapiens |
| 17 | AAH11674 | 493 | 66 | BC011674 Similar to procollagen- | Homo sapiens |
| | | | | lysine, 2-oxoglutarate 5- | |
| | | | | dioxygenase 3 | |
| 17 | AC004876_4 | 493 | 66 | AC004876 lysyl hydroxylase 3 | Homo sapiens |
| 17 | AF207069 | 493 | 66 | lysyl hydroxylase 3 | Homo sapiens |
| 18 | AAH16765 | 1778 | 99 | BC016765 Unknown (protein for | Homo sapiens |
| | | | | MGC: 22663) | |
| 18 | AF153882_1 | 1785 | 100 | AF153882 reversion-induced LIM | Homo sapiens |
| | | | | protein | |
| 18 | AF154335_1 | 1785 | 100 | AF154335 reversion-induced LIM | Homo sapiens |
| | | | | protein | |
| 19 | AF023268 | 1150 | 74 | metaxin | Homo sapiens |
| 19 | BC023071 | 1050 | 68 | metaxin | Mus musculus |
| 19 | U46920 | 1150 | 74 | metaxin | Homo sapiens |
| 20 | AF033815 | 363 | 34 | Pr180 | Mason-Pfizer |
| | | | | | monkey virus |
| 20 | AF193053_1 | 575 | 100 | AF193053 unknown | Homo sapiens |
| 20 | Y10392 | 500 | 43 | protease | Human |
| | | | | | endogenous |
| | | | | | retrovirus K |
| 21 | AF318351_1 | 3211 | 100 | AF318351 unknown | Homo sapiens |
| 21 | AF318377_1 | 3211 | 100 | AF318377 unknown | Homo sapiens |
| 21 | AX106709 | 3211 | 100 | unnamed protein product | Homo sapiens |
| 22 | AL357374 | 2678 | 100 | bA353C18.3.2 (splicing factor | Homo sapiens |
| | | | | CC1.3, isoform 2 (CC1.4)) | |
| 22 | AY061882 | 2663 | 99 | transcription coactivator CAPER | Mus musculus |
| 22 | L10911 | 2678 | 100 | splicing factor | Homo sapiens |
| 23 | AAH08807 | 2069 | 90 | BC008807 keratin 6A | Homo sapiens |
| 23 | AAH14152 | 2069 | 90 | BC014152 Similar to keratin 6A | Homo sapiens |
| 23 | L42583 | 2069 | 90 | keratin type II | Homo sapiens |
| 24 | AK000496 | 301 | 72 | unnamed protein product | Homo sapiens |
| 24 | AK025116 | 271 | 67 | unnamed protein product | Homo sapiens |
| 24 | AK090511 | 282 | 60 | unnamed protein product | Homo sapiens |
| 25 | AAH16842 | 2431 | 99 | BC016842 Unknown (protein for | Homo sapiens |
| | | | | MGC: 10135) | |
| 25 | AK027643 | 2435 | 100 | unnamed protein product | Homo sapiens |
| 25 | AL834398 | 2529 | 99 | hypothetical protein | Homo sapiens |
| 26 | AAH02563 | 1176 | 100 | BC002563 Similar to zinc/iron | Homo sapiens |
| | | | | regulated transporter-like | |
| 26 | AAH07886 | 1176 | 100 | BC007886 Unknown (protein for | Homo sapiens |
| | | | | MGC: 14180) | |
| 26 | AAH14303 | 1176 | 100 | BC014303 Unknown (protein for | Homo sapiens |
| | | | | MGC: 16418) | |
| 27 | AF056732 | 3342 | 100 | prostaglandin transporter | Homo sapiens |
| 27 | BC041140 | 3453 | 99 | solute carrier family 21 | Homo sapiens |
| | | | | (prostaglandin transporter), | |
| | | | | member 2 | |
| 27 | U70867 | 3342 | 100 | prostaglandin transporter hPGT | Homo sapiens |
| 28 | AAH15206 | 335 | 100 | BC015206 guanine nucleotide | Homo sapiens |
| | | | | binding protein 10 | |
| 28 | AAH16319 | 335 | 100 | BC016319 guanine nucleotide | Homo sapiens |
| | | | | binding protein 10 | |
| 28 | AF493877_1 | 335 | 100 | AF493877 guanine nucleotide | Homo sapiens |
| | | | | binding protein gamma 10 | |
| 29 | D26307 | 1148 | 66 | Jun-D | Rattus |
| | | | | | norvegicus |
| 29 | X51346 | 1419 | 97 | jun-D gene product (AA 1-303) | Homo sapiens |
| 29 | X56681 | 1388 | 96 | junD protein | Homo sapiens |
| 30 | AY081680 | 693 | 100 | chlorophyll a/b-binding protein | Arabidopsis |
| | | | | CP29 | thaliana |
| 30 | AY133566 | 693 | 100 | At5g01530/F7A7_50 | Arabidopsis |
| | | | | | thaliana |
| 30 | BT000363 | 693 | 100 | chlorophyll a/b-binding protein | Arabidopsis |
| | | | | CP29 | thaliana |
| 31 | AY086905 | 622 | 100 | photosystem II type I | Arabidopsis |
| | | | | chlorophyll a/b binding protein, | thaliana |
| | | | | putative | |
| 31 | AY128345 | 622 | 100 | photosystem II type I chlorophyll | Arabidopsis |
| | | | | a/b binding protein, putative | thaliana |
| 31 | BT000726 | 622 | 100 | putative photosystem II type I | Arabidopsis |
| | | | | chlorophyll a/b binding protein | thaliana |
| 32 | AF177203_1 | 847 | 98 | AF177203 cerebral cell adhesion | Homo sapiens |
| | | | | molecule | |
| 32 | AK074519 | 872 | 100 | unnamed protein product | Homo sapiens |
| 32 | AK074523 | 872 | 100 | unnamed protein product | Homo sapiens |
| 33 | AY050935 | 497 | 100 | putative photosystem II type I | Arabidopsis |
| | | | | chlorophyll a/b binding protein | thaliana |
| 33 | AY058180 | 497 | 100 | At1g29930/F1N18_23 | Arabidopsis |
| | | | | | thaliana |
| 33 | AY091169 | 497 | 100 | putative chlorophyll a/b-binding | Arabidopsis |
| | | | | protein | thaliana |
| 34 | AF178954 | 2149 | 88 | monocarboxylate transporter 4 | Mus musculus |
| 34 | U81800 | 2399 | 100 | monocarboxylate transporter | Homo sapiens |
| 34 | U87627 | 2164 | 88 | putative monocarboxylate | Rattus |
| | | | | transporter | norvegicus |
| 35 | AAH18345 | 173 | 100 | BC018345 Unknown (protein for | Homo sapiens |
| | | | | MGC: 8802) | |
| 35 | AF237776_1 | 173 | 100 | AF237776 MAP kinase-interacting | Homo sapiens |
| | | | | kinase 2b | |
| 35 | AL137565 | 173 | 100 | hypothetical protein | Homo sapiens |
| 36 | AF006013 | 3773 | 99 | dishevelled 3 | Homo sapiens |
| 36 | BC032459 | 3784 | 99 | dishevelled, dsh homolog 3 | Homo sapiens |
| | | | | (Drosophila) | |
| 36 | U75651 | 3806 | 100 | dishevelled 3 | Homo sapiens |
| 37 | AK000268 | 2659 | 100 | unnamed protein product | Homo sapiens |
| 37 | AK053678 | 1598 | 71 | unnamed protein product | Mus musculus |
| 37 | L00193 | 1342 | 58 | epidermal keratin subunit I | Mus musculus |
| 38 | AX354357 | 840 | 100 | unnamed protein product | Homo sapiens |
| 38 | BC002198 | 243 | 42 | Similar to hypothetical protein | Mus musculus |
| | | | | FLJ20093 | |
| 38 | BC039642 | 658 | 75 | Unknown (protein for | Mus musculus |
| | | | | IMAGE: 5352548) | |
| 39 | AF012348 | 857 | 84 | smooth muscle gamma actin | Gallus |
| | | | | | gallus |
| 39 | S63494 | 857 | 84 | gamma-actin | Gallus |
| | | | | | gallus |
| 39 | U20365 | 857 | 84 | smooth muscle gamma-actin | Mus musculus |
| 40 | AB023209 | 2102 | 61 | KIAA0992 protein | Homo sapiens |
| 40 | AF077041_1 | 2057 | 100 | AF077041 SIH002 | Homo sapiens |
| 40 | AF151909_1 | 2161 | 100 | AF151909 CGI-151 protein | Homo sapiens |
| 41 | AF287271_1 | 714 | 100 | AF287271 ribosomal protein L23 | Mus musculus |
| 41 | AF401577_1 | 714 | 100 | AF401577 ribosomal protein L23 | Ictalurus |
| | | | | | punctatus |
| 41 | BC025918 | 714 | 100 | ribosomal protein L23 | Mus musculus |
| 42 | U43916 | 817 | 100 | TMP | Homo sapiens |
| 42 | Y07909 | 817 | 100 | progression associated protein | Homo sapiens |
| 42 | Z50751 | 817 | 100 | B4B | Homo sapiens |
| 43 | AAH09647 | 2778 | 99 | BC009647 Unknown (protein for | Homo sapiens |
| | | | | MGC: 5096) | |
| 43 | M13078 | 2773 | 99 | alkaline phosphatase precursor | Homo sapiens |
| 43 | M19159 | 2780 | 100 | placental alkaline phosphatase-1 | Homo sapiens |
| 44 | AF326591 | 2240 | 100 | fenestrated-endothelial linked | Homo sapiens |
| | | | | structure protein | |
| 44 | AF348827 | 2232 | 99 | PV1 protein | Homo sapiens |
| 44 | AL834363 | 2304 | 99 | hypothetical protein | Homo sapiens |
| 45 | AAH11686 | 5877 | 100 | BC011686 damage-specific DNA | Homo sapiens |
| | | | | binding protein 1 (127 kD) | |
| 45 | L40326 | 5877 | 100 | X-associated protein 1 | Homo sapiens |
| 45 | U18299 | 5877 | 100 | DDBa p127 | Homo sapiens |
| 46 | U66661 | 1330 | 100 | GABA-A receptor epsilon subunit | Homo sapiens |
| 46 | Y07637 | 1330 | 100 | putative GABA-gated chloride | Homo sapiens |
| | | | | channel | |
| 46 | Y09765 | 1330 | 100 | GABA receptor epsilon subunit | Homo sapiens |
| 47 | AF380181_1 | 11506 | 99 | AF380181 SON DNA binding protein | Homo sapiens |
| | | | | isoform C | |
| 47 | AF380184_1 | 12117 | 99 | AF380184 SON DNA binding protein | Homo sapiens |
| | | | | isoform F | |
| 47 | AY026895 | 11842 | 97 | NREBP | Homo sapiens |
| 48 | A20470 | 2142 | 99 | preA-PAI-2 | synthetic |
| | | | | | construct |
| 48 | AAH12609 | 2148 | 100 | BC012609 Similar to serine (or | Homo sapiens |
| | | | | cysteine) proteinase inhibitor, | |
| | | | | clade B (ovalbumin), member 2 | |
| 48 | Y00630 | 2139 | 99 | PAI-2 precursor (AA −22 to 393) | Homo sapiens |
| 49 | AB037819 | 7936 | 100 | KIAA1398 protein | Homo sapiens |
| 49 | AL132765 | 5785 | 95 | bA462D18.3.2 (ribosome binding | Homo sapiens |
| | | | | protein 1 (dog 180 kDa homolog) | |
| | | | | (KIAA1398) (isoform 2)) | |
| 49 | X87224 | 6518 | 84 | ribosome receptor | Canis |
| | | | | | familiaris |
| 50 | AAH00716 | 1700 | 99 | BC000716 seryl-tRNA synthetase | Homo sapiens |
| 50 | D49914 | 1700 | 99 | Seryl tRNA Synthetase | Homo sapiens |
| 50 | X91257 | 1700 | 99 | seryl-tRNA synthetase | Homo sapiens |
| 51 | D29640 | 4698 | 57 | KIAA0051 | Homo sapiens |
| 51 | L33075 | 4698 | 57 | ras GTPase-activating-like | Homo sapiens |
| | | | | protein | |
| 51 | U51903 | 8003 | 100 | RasGAP-related protein | Homo sapiens |
| 52 | AAH17338 | 2576 | 99 | BC017338 fucosidase, alpha-L-1, | Homo sapiens |
| | | | | tissue | |
| 52 | M29877 | 2581 | 100 | alpha-L-fucosidase precursor (EC | Homo sapiens |
| | | | | 3.2.1.5) | |
| 52 | M80815 | 2576 | 99 | alpha-L-fucosidase | Homo sapiens |
| 53 | AAH03067 | 3601 | 100 | BC003067 growth arrest and DNA- | Homo sapiens |
| | | | | damage-inducible 34 | |
| 53 | AK001361 | 3568 | 99 | unnamed protein product | Homo sapiens |
| 53 | U83981 | 3601 | 100 | apoptosis associated protein | Homo sapiens |
| 54 | AAH02717 | 379 | 77 | BC002717 Similar to chorionic | Homo sapiens |
| | | | | somatomammotropin hormone 1 | |
| | | | | (placental lactogen) | |
| 54 | AAH05921 | 379 | 77 | BC005921 chorionic | Homo sapiens |
| | | | | somatomammotropin hormone 1 | |
| | | | | (placental lactogen) | |
| 54 | AAH20756 | 379 | 77 | BC020756 chorionic | Homo sapiens |
| | | | | somatomammotropin hormone 1 | |
| | | | | (placental lactogen) | |
| 55 | M57939 | 2349 | 100 | ribonucleoprotein antigen | Homo sapiens |
| 55 | X04654 | 2385 | 97 | 70 K protein (AA 1-614) | Homo sapiens |
| 55 | X84841 | 2349 | 100 | 68 kDa (U1) ribonucleoprotein | Homo sapiens |
| 56 | AAH09945 | 3737 | 99 | BC009945 Unknown (protein for | Homo sapiens |
| | | | | IMAGE: 4123572) | |
| 56 | AB018289 | 5502 | 100 | KIAA0746 protein | Homo sapiens |
| 56 | AK030487 | 4620 | 87 | unnamed protein product | Mus musculus |
| 57 | AJ422147 | 664 | 100 | C15 protein | Homo sapiens |
| 57 | AX352692 | 664 | 100 | unnamed protein product | Homo sapiens |
| 57 | AX357061 | 664 | 100 | unnamed protein product | Homo sapiens |
| 59 | AAH01501 | 1520 | 100 | BC001501 Unknown (protein for | Homo sapiens |
| | | | | MGC: 2233) | |
| 59 | AAH14078 | 1520 | 100 | BC014078 cyclin D1 (PRAD1: | Homo sapiens |
| | | | | parathyroid adenomatosis 1) | |
| 59 | BC025302 | 1520 | 100 | cyclin D1 (PRAD1: parathyroid | Homo sapiens |
| | | | | adenomatosis 1) | |
| 60 | BC036285 | 1936 | 100 | protein kinase, cAMP-dependent, | Homo sapiens |
| | | | | regulatory, type I, alpha (tissue | |
| | | | | specific extinguisher 1) | |
| 60 | M18468 | 1936 | 100 | cAMP-dependent protein kinase | Homo sapiens |
| | | | | regulatory subunit type 1 | |
| 60 | M33336 | 1936 | 100 | cAMP-dependent protein kinase | Homo sapiens |
| | | | | type I-alpha subunit | |
| 61 | BC024165 | 600 | 100 | polymerase (RNA) II (DNA | Homo sapiens |
| | | | | directed) polypeptide J (13.3 kD) | |
| 61 | X82385 | 600 | 100 | RNA polymerase II subunit | Homo sapiens |
| 61 | X98433 | 600 | 100 | RNA polymerase II subunit | Homo sapiens |
| 62 | AL138761 | 8033 | 99 | bA16H23.2 (collagen, type XVII, | Homo sapiens |
| | | | | alpha 1 (BP180)) | |
| 62 | M91669 | 8228 | 100 | autoantigen | Homo sapiens |
| 62 | U76604 | 8037 | 99 | 180 kDa bullous pemphigoid | Homo sapiens |
| | | | | antigen 2/type XVII collagen | |
| 63 | AAH03575 | 2373 | 100 | BC003575 mannosyl (alpha-1,3-)- | Homo sapiens |
| | | | | glycoprotein beta-1,2-N- | |
| | | | | acetylglucosaminyltransferase | |
| 63 | M55621 | 2363 | 99 | N-acetylglucosaminyltransferase I | Homo sapiens |
| 63 | M61829 | 2373 | 100 | alpha-1,3-mannosyl-glycoprotein | Homo sapiens |
| | | | | beta-1,2-N- | |
| | | | | acetylglucosaminyltransferase | |
| 64 | L13923 | 16639 | 99 | fibrillin | Homo sapiens |
| 64 | L28748 | 16362 | 97 | putative | Bos taurus |
| 64 | X63556 | 17359 | 100 | fibrillin | Homo sapiens |
| 65 | AAH02576 | 3661 | 100 | BC002576 matrix metalloproteinase | Homo sapiens |
| | | | | 2 (gelatinase A, 72 kD gelatinase, | |
| | | | | 72 kD type IV collagenase) | |
| 65 | J03210 | 3584 | 100 | collagenase type IV precursor | Homo sapiens |
| 65 | M55593 | 3661 | 100 | type IV collagenase | Homo sapiens |
| 66 | AAH22087 | 2466 | 99 | BC022087 Similar to CD36 antigen | Homo sapiens |
| | | | | (collagen type I receptor, | |
| | | | | thrombospondin receptor)-like 1 | |
| 66 | AF467889_1 | 2380 | 86 | AF467889 high density lipoprotein | Sus scrofa |
| | | | | receptor SR-BI | |
| 66 | Z22555 | 2684 | 100 | CLA-1 | Homo sapiens |
| 67 | AF106202_1 | 1249 | 100 | AF106202 endothelial cell protein | Homo sapiens |
| | | | | C receptor precursor | |
| 67 | AF375468_1 | 1249 | 100 | AF375468 endothelial protein C | Homo sapiens |
| | | | | receptor | |
| 67 | X89079 | 1249 | 100 | centrocyclin | Homo sapiens |
| 68 | AF153603_1 | 666 | 100 | AF153603 TSC-22 related protein | Homo sapiens |
| 68 | AF183393_1 | 666 | 100 | AF183393 TSC-22-like Protein | Homo sapiens |
| 68 | AF228339 | 666 | 100 | glucocorticoid-induced GILZ | Homo sapiens |
| 69 | AF111168 | 3586 | 99 | unknown | Homo sapiens |
| 69 | AK075445 | 2876 | 99 | unnamed protein product | Homo sapiens |
| 69 | AX136231 | 2876 | 99 | unnamed protein product | Homo sapiens |
| 70 | AAH11234 | 1220 | 100 | BC011234 Similar to splicing | Homo sapiens |
| | | | | factor 30, survival of motor | |
| | | | | neuron-related | |
| 70 | AF083385 | 1220 | 100 | 30 kDa splicing factor; SPF 30 | Homo sapiens |
| 70 | AF107463 | 1220 | 100 | splicing factor | Homo sapiens |
| 71 | AAH10240 | 1921 | 100 | BC010240 cathepsin B | Homo sapiens |
| 71 | L16510 | 1912 | 99 | cathepsin B | Homo sapiens |
| 71 | M14221 | 1915 | 99 | preprocathepsin B | Homo sapiens |
| 72 | AK056033 | 398 | 83 | unnamed protein product | Homo sapiens |
| 73 | AE014836_21 | 182 | 23 | AE014836 hypothetical protein | Plasnodium |
| | | | | | falciparun |
| | | | | | 3D7 |
| 73 | AK033354 | 174 | 24 | unnamed protein product | Mus musculus |
| 74 | AF222743_1 | 2415 | 100 | AF222743 Sec61 alpha-1 | Mus musculus |
| 74 | BC003707 | 2415 | 100 | sec61 homolog | Mus musculus |
| 74 | M96630 | 2415 | 100 | sec61-like protein | Rattus sp. |
| 75 | AF332086_1 | 3193 | 96 | AF332086 Rad21 | Mus musculus |
| 75 | D38551 | 3284 | 100 | The ha1237 gene product is | Homo sapiens |
| | | | | related to S.pombe rad21 gene | |
| | | | | product. | |
| 75 | X98294 | 3275 | 99 | HR21spA | Homo sapiens |
| 76 | AAH16325 | 925 | 100 | BC016325 ADP-ribosylation factor 4 | Homo sapiens |
| 76 | AF493883_1 | 925 | 100 | AF493883 ADP-ribosylation factor | Homo sapiens |
| | | | | protein 4 | |
| 76 | BC022866 | 925 | 100 | ADP-ribosylation factor 4 | Homo sapiens |
| 77 | AAH18201 | 763 | 100 | BC018201 Unknown (protein for | Homo sapiens |
| | | | | MGC: 21505) | |
| 77 | AF217965_1 | 763 | 100 | AF217965 unknown | Homo sapiens |
| 77 | AF217972_1 | 763 | 100 | AF217972 unknown | Homo sapiens |
| 78 | AAH00211 | 1127 | 97 | BC000211 eukaryotic translation | Homo sapiens |
| | | | | elongation factor 1 beta 2 | |
| 78 | AAH04931 | 1127 | 97 | BC004931 eukaryotic translation | Homo sapiens |
| | | | | elongation factor 1 beta 2 | |
| 78 | X60489 | 1127 | 97 | elongation factor-1-beta | Homo sapiens |
| 79 | D82984 | 1758 | 93 | C/EBP alpha protein | Bos taurus |
| 79 | U34070 | 1857 | 96 | CCAAT/enhancer binding protein | Homo sapiens |
| | | | | alpha | |
| 79 | Y11525 | 1927 | 100 | CCAAT/enhancer binding protein | Homo sapiens |
| | | | | alpha | |
| 80 | AK025116 | 341 | 71 | unnamed protein product | Homo sapiens |
| 80 | AK090511 | 358 | 72 | unnamed protein product | Homo sapiens |
| 80 | AK096373 | 341 | 70 | unnamed protein product | Homo sapiens |
| 81 | AAH13963 | 2458 | 100 | BC013963 Similar to adenylyl | Homo sapiens |
| | | | | cyclase-associated protein | |
| 81 | AAH17196 | 2458 | 100 | BC017196 adenylyl cyclase- | Homo sapiens |
| | | | | associated protein | |
| 81 | M98474 | 2458 | 100 | CAP protein | Homo sapiens |
| 82 | AAH02464 | 2175 | 99 | BC002464 coagulation factor II | Homo sapiens |
| | | | | (thrombin) receptor | |
| 82 | AF391809_1 | 2179 | 99 | AF391809 coagulation factor II | Homo sapiens |
| | | | | (thrombin) receptor | |
| 82 | M62424 | 2187 | 100 | thrombin receptor | Homo sapiens |
| 83 | AF085250 | 759 | 100 | calmodulin | Perca |
| | | | | | flavescens |
| 83 | D83350 | 759 | 100 | calmodulin | Anas |
| | | | | | platyrhynchos |
| 83 | K01945 | 759 | 100 | calmodulin (cDNA clone 71) | Xenopus |
| | | | | | laevis |
| 84 | AL050265 | 2193 | 100 | hypothetical protein | Homo sapiens |
| 84 | BC033475 | 2128 | 96 | similar to TAR DNA-binding | Mus musculus |
| | | | | protein-43 (TDP-43) | |
| 84 | U23731 | 2193 | 100 | TAR DNA-binding protein-43 | Homo sapiens |
| 85 | AAH01495 | 1673 | 99 | BC001495 chromosome 1 open | Homo sapiens |
| | | | | reading frame 8 | |
| 85 | AAH16374 | 1697 | 100 | BC016374 chromosome 1 open | Homo sapiens |
| | | | | reading frame 8 | |
| 85 | AF290615_1 | 1673 | 99 | AF290615 liver membrane-bound | Homo sapiens |
| | | | | protein | |
| 86 | AK025047 | 319 | 69 | unnamed protein product | Homo sapiens |
| 86 | AK090511 | 327 | 70 | unnamed protein product | Homo sapiens |
| 86 | AK092450 | 358 | 69 | unnamed protein product | Homo sapiens |
| 87 | AAH00642 | 683 | 99 | BC000642 MY047 protein | Homo sapiens |
| 87 | AF063605 | 688 | 100 | brain my047 protein | Homo sapiens |
| 87 | AX136119 | 683 | 99 | unnamed protein product | Homo sapiens |
| 88 | AF035947_1 | 1394 | 100 | AF035947 cytokine-inducible | Homo sapiens |
| | | | | inhibitor of signalling type 1b | |
| 88 | AF132297_1 | 1332 | 99 | AF132297 cytokine-inducible SH2- | Homo sapiens |
| | | | | containing protein | |
| 88 | BC031590 | 1321 | 100 | cytokine inducible SH2-containing | Homo sapiens |
| | | | | protein | |
| 89 | AF151018_1 | 863 | 100 | AF151018 HSPC184 | Homo sapiens |
| 89 | AK012047 | 863 | 100 | unnamed protein product | Mus musculus |
| 89 | AK075227 | 863 | 100 | unnamed protein product | Homo sapiens |
| 90 | AK025116 | 323 | 68 | unnamed protein product | Homo sapiens |
| 90 | AK090929 | 316 | 70 | unnamed protein product | Homo sapiens |
| 90 | U22376 | 203 | 61 | alternatively spliced product | Homo sapiens |
| | | | | using exon 13A | |
| 91 | AAH09357 | 782 | 98 | BC009357 transgelin 2 | Homo sapiens |
| 91 | AY007127 | 887 | 98 | similar to Homo sapiens mRNA for | |
| | | | | KIAA0120 gene with GenBank | |
| | | | | Accession Number D21261.1 | |
| 91 | D21261 | 782 | 98 | similar to human 22 kDa, SM22 mRNA | Homo sapiens |
| | | | | (HUM22SM). | |
| 92 | AE005963 | 413 | 91 | TonB-dependent receptor | Caulobacter |
| | | | | | crescentus |
| | | | | | CB15 |
| 92 | AE005978 | 307 | 67 | TonB-dependent receptor | Caulobacter |
| | | | | | crescentus |
| | | | | | CB15 |
| 92 | AE005979 | 307 | 67 | TonB-dependent receptor | Caulobacter |
| | | | | | crescentus |
| | | | | | CB15 |
| 93 | AAH14006 | 367 | 81 | BC014006 Similar to 6- | Homo sapiens |
| | | | | phosphogluconolactonase | |
| 93 | AJ243972 | 367 | 81 | 6-phosphogluconolactonase | Homo sapiens |
| 93 | BC006594 | 325 | 71 | RIKEN cDNA 1110030K05 gene | Mus musculus |
| 94 | AAH03125 | 2055 | 99 | BC003125 Unknown (protein for | Homo sapiens |
| | | | | MGC: 3222) | |
| 94 | AK027877 | 2055 | 99 | unnamed protein product | Homo sapiens |
| 94 | AL136916 | 2055 | 99 | hypothetical protein | Homo sapiens |
| 95 | AAH03681 | 2956 | 100 | BC003681 Unknown (protein for | Homo sapiens |
| | | | | IMAGE: 3453235) | |
| 95 | AL096828 | 3008 | 100 | dJ963E22.1 (Novel protein similar | Homo sapiens |
| | | | | to NY-REN-2 Antigen) | |
| 95 | AX400070 | 2878 | 96 | unnamed protein product | Homo sapiens |
| 96 | AF161372_1 | 872 | 100 | AF161372 HSPC254 | Homo sapiens |
| 96 | BC021602 | 519 | 98 | Unknown (protein for | Mus musculus |
| | | | | IMAGE: 5102170) | |
| 96 | BC021603 | 519 | 98 | Unknown (protein for | Mus musculus |
| | | | | IMAGE: 5102465) | |
| 97 | AAH11714 | 476 | 100 | BC011714 Similar to heterogeneous | Homo sapiens |
| | | | | nuclear ribonucleoprotein D-like | |
| 97 | AB017020 | 476 | 100 | JKTBP | Mus musculus |
| 97 | BC021374 | 476 | 100 | heterogeneous nuclear | Mus musculus |
| | | | | ribonucleoprotein D-like | |
| 98 | AAH02673 | 1063 | 100 | BC002673 protein kinase H11; | Homo sapiens |
| | | | | small stress protein-like protein | |
| | | | | HSP22 | |
| 98 | AF191017_1 | 1063 | 100 | AF191017 E2IG1 | Homo sapiens |
| 98 | AF250138_1 | 1063 | 100 | AF250138 small stress protein- | Homo sapiens |
| | | | | like protein HSP22 | |
| 99 | AAH05033 | 2990 | 99 | BC005033 Unknown (protein for | Homo sapiens |
| | | | | MGC: 12692) | |
| 99 | AAH15620 | 2990 | 99 | BC015620 Unknown (protein for | Homo sapiens |
| | | | | IMAGE: 4843317) | |
| 99 | D89980 | 2990 | 99 | alpha actinin 4 | Homo sapiens |
| 100 | AAH11908 | 1787 | 100 | BC011908 Unknown (protein for | Homo sapiens |
| | | | | MGC: 20321) | |
| 100 | AAH14564 | 1787 | 100 | BC014564 Unknown (protein for | Homo sapiens |
| | | | | MGC: 2063) | |
| 100 | AAH18695 | 1787 | 100 | BC018695 Unknown (protein for | Homo sapiens |
| | | | | MGC: 3838) | |
| 101 | AAH12977 | 1363 | 100 | BC012977 nuclear LIM interactor- | Homo sapiens |
| | | | | interacting factor | |
| 101 | AF229162_1 | 1363 | 100 | AF229162 nuclear LIM interactor- | Homo sapiens |
| | | | | interacting factor | |
| 101 | AF229163 | 1363 | 100 | nuclear LIM interactor- | Homo sapiens |
| | | | | interacting factor | |
| 102 | AAH09718 | 355 | 76 | BC009718 epithelial membrane | Homo sapiens |
| | | | | protein 3 | |
| 102 | U52101 | 355 | 76 | YMP | Homo sapiens |
| 102 | U87947 | 355 | 76 | hematopoietic neural membrane | Homo sapiens |
| | | | | protein | |
| 103 | AF162781_1 | 4987 | 88 | AF162781 SH2-containing inositol | Mus musculus |
| | | | | 5-phosphatase 2 | |
| 103 | L36818 | 6041 | 99 | 51C protein | Homo sapiens |
| 103 | Y14385 | 5272 | 92 | inositol polyphosphate 5- | Homo sapiens |
| | | | | phosphatase | |
| 104 | AAH17369 | 1011 | 92 | BC017369 splicing factor, | Homo sapiens |
| | | | | arginine/serine-rich 7 (35 kD) | |
| 104 | AAH17908 | 1011 | 92 | BC017908 splicing factor, | Homo sapiens |
| | | | | arginine/serine-rich 7 (35 kD) | |
| 104 | BC022328 | 1011 | 92 | splicing factor, arginine/serine- | Homo sapiens |
| | | | | rich 7 (35 kD) | |
| 105 | AAH14323 | 1209 | 99 | BC014323 Similar to accessory | Homo sapiens |
| | | | | proteins BAP31/BAP29 | |
| 105 | X81109 | 1213 | 100 | tumor-associated antigen | Homo sapiens |
| 105 | Z31696 | 1213 | 100 | CDM | Homo sapiens |
| 106 | AAH15763 | 3102 | 100 | BC015763 hypothetical protein | Homo sapiens |
| | | | | FLJ20258 | |
| 106 | AK000265 | 3093 | 99 | unnamed protein product | Homo sapiens |
| 106 | AK075098 | 2998 | 99 | unnamed protein product | Homo sapiens |
| 107 | AAH07858 | 2250 | 100 | BC007858 inhibin, beta A (activin | Homo sapiens |
| | | | | A, activin AB alpha polypeptide) | |
| 107 | M13436 | 2250 | 100 | beta-A inhibin | Homo sapiens |
| 107 | X57578 | 2250 | 100 | activin beta-A subunit | Homo sapiens |
| 108 | AK027598 | 3474 | 100 | unnamed protein product | Homo sapiens |
| 108 | AX481758 | 3498 | 99 | unnamed protein product | Homo sapiens |
| 108 | AX481760 | 3498 | 99 | unnamed protein product | Homo sapiens |
| 109 | AAH15958 | 1926 | 86 | BC015958 Unknown (protein for | Homo sapiens |
| | | | | MGC: 15290) | |
| 109 | AJ401269 | 1926 | 86 | polyadenylate-binding protein 1 | Bos taurus |
| 109 | AK044829 | 1922 | 85 | unnamed protein product | Mus musculus |
| 110 | AAH10623 | 3729 | 96 | BC010623 Similar to nuclear | Homo sapiens |
| | | | | factor (erythroid-derived 2)-like 1 | |
| 110 | AK090459 | 4032 | 100 | FLJ00380 protein | Homo sapiens |
| 110 | X77366 | 3926 | 100 | hbZ17 | Homo sapiens |
| 111 | AAH03177 | 3500 | 100 | BC003177 KIAA1536 protein | Homo sapiens |
| 111 | AK027881 | 3480 | 99 | unnamed protein product | Homo sapiens |
| 111 | AL136895 | 3500 | 100 | hypothetical protein | Homo sapiens |
| 112 | AY054301 | 4981 | 94 | type III preprocollagen alpha 1 | Homo sapiens |
| | | | | chain | |
| 112 | BC028178 | 6603 | 100 | Similar to collagen, type III, | Homo sapiens |
| | | | | alpha 1 (Ehlers-Danlos syndrome | |
| | | | | type IV, autosomal dominant) | |
| 112 | X14420 | 4981 | 94 | prepro-alpha-1 type 3 collagen | Homo sapiens |
| 113 | AAH11819 | 3473 | 99 | BC011819 DEAD/H (Asp-Glu-Ala- | Homo sapiens |
| | | | | Asp/His) box polypeptide 3 | |
| 113 | AF000982 | 3476 | 100 | dead box, X isoform | Homo sapiens |
| 113 | AF000983 | 3476 | 100 | dead box, X isoform | Homo sapiens |
| 114 | AAH14959 | 1160 | 100 | BC014959 RING1 and YY1 binding | Homo sapiens |
| | | | | protein | |
| 114 | AF179286_1 | 1160 | 100 | AF179286 death effector domain- | Homo sapiens |
| | | | | associated factor | |
| 114 | BC036459 | 1156 | 99 | RING1 and YY1 binding protein | Homo sapiens |
| 115 | AK002791 | 282 | 81 | unnamed protein product | Mus musculus |
| 115 | AK003292 | 282 | 81 | unnamed protein product | Mus musculus |
| 115 | BC016191 | 282 | 81 | RIKEN cDNA 0610038D11 gene | Mus musculus |
| 116 | AAH08442 | 1106 | 100 | BC008442 Similar to transmembrane | Homo sapiens |
| | | | | 4 superfamily member 1 | |
| 116 | AAH10166 | 1106 | 100 | BC010166 Similar to transmembrane | Homo sapiens |
| | | | | 4 superfamily member 1 | |
| 116 | M90657 | 1106 | 100 | L6 | Homo sapiens |
| 117 | AAH10354 | 1764 | 100 | BC010354 PTD010 protein | Homo sapiens |
| 117 | AB009685 | 1764 | 100 | DERP2 | Homo sapiens |
| 117 | AF131820 | 1764 | 100 | Unknown | Homo sapiens |
| 118 | AF217787 | 1172 | 99 | extracellular glutathione | Homo sapiens |
| | | | | peroxidase | |
| 118 | D00632 | 1192 | 99 | glutathione peroxidase | Homo sapiens |
| 118 | X58295 | 1192 | 99 | glutathione peroxidase | Homo sapiens |
| 119 | AAH00531 | 939 | 97 | BC000531 insulin-like growth | Homo sapiens |
| | | | | factor 2 (somatomedin A) | |
| 119 | AF517226_1 | 939 | 97 | AF517226 insulin-like growth | Homo sapiens |
| | | | | factor 2 (somatomedin A) | |
| 119 | M17863 | 942 | 97 | preproinsulin-like growth factor | Homo sapiens |
| | | | | II, domains A-E | |
| 120 | AJ310544 | 2183 | 100 | EGLN2 protein | Homo sapiens |
| 120 | AX553864 | 2183 | 100 | unnamed protein product | Homo sapiens |
| 120 | BC036051 | 2183 | 100 | Similar to egl nine homolog 2 (C. elegans) | Homo sapiens |
| 121 | AAH00867 | 915 | 98 | BC000867 PRO0659 protein | Homo sapiens |
| 121 | AL834135 | 1118 | 99 | hypothetical protein | Homo sapiens |
| 121 | AX405861 | 1118 | 99 | unnamed protein product | Homo sapiens |
| 122 | AB051480 | 1087 | 60 | KIAA1693 protein | Homo sapiens |
| 122 | AL136890 | 1741 | 83 | hypothetical protein | Homo sapiens |
| 122 | AX402334 | 1084 | 60 | unnamed protein product | Homo sapiens |
| 123 | AB037755 | 4896 | 99 | KIAA1334 protein | Homo sapiens |
| 123 | AF155135_1 | 4845 | 99 | AF155135 novel retinal pigment | Homo sapiens |
| | | | | epithelial cell protein | |
| 123 | AF202315_1 | 4141 | 85 | AF202315 ankycorbin | Mus musculus |
| 124 | AK090929 | 329 | 76 | unnamed protein product | Homo sapiens |
| 124 | AK097965 | 338 | 77 | unnamed protein product | Homo sapiens |
| 124 | BC033959 | 336 | 70 | similar to Alu subfamily SQ | Homo sapiens |
| | | | | sequence contamination warning | |
| | | | | entry | |
| 125 | AAH12444 | 1319 | 100 | BC012444 Similar to chloride | Homo sapiens |
| | | | | intracellular channel 4 | |
| 125 | AF097330_1 | 1305 | 99 | AF097330 H1 chloride channel; | Homo sapiens |
| | | | | p64H1; CLIC4 | |
| 125 | AL117424 | 1319 | 100 | hypothetical protein | Homo sapiens |
| 126 | AB034747 | 904 | 100 | small integral membrane protein | Homo sapiens |
| | | | | of lysosome/late endosome | |
| 126 | AF171100 | 812 | 85 | TBX1 protein | Mus musculus |
| 126 | BC018559 | 812 | 85 | LPS-induced TNF-alpha factor | Mus musculus |
| 127 | AAH10645 | 1737 | 100 | BC010645 Similar to | Homo sapiens |
| | | | | sialyltransferase 4C (beta- | |
| | | | | galactosidase alpha-2,3- | |
| | | | | sialytransferase) | |
| 127 | AY040826 | 1715 | 97 | alpha 2,3-sialyltransferase IV | Homo sapiens |
| | | | | type B+18 | |
| 127 | L23767 | 1710 | 100 | a2,3 sialyltransferse | Homo sapiens |
| 128 | AAH09329 | 639 | 64 | BC009329 triosephosphate | Homo sapiens |
| | | | | isomerase 1 | |
| 128 | AAH11611 | 639 | 64 | BC011611 Similar to | Homo sapiens |
| | | | | triosephosphate isomerase 1 | |
| 128 | AAH15100 | 639 | 64 | BC015100 triosephosphate | Homo sapiens |
| | | | | isomerase 1 | |
| 129 | AAH18986 | 1064 | 91 | BC018986 Unknown (protein for | Homo sapiens |
| | | | | MGC: 20092) | |
| 129 | AB059653 | 1060 | 90 | prostaglandin dehydrogenase I | Macaca |
| | | | | | fascicularis |
| 129 | L76465 | 1057 | 90 | NAD+-dependent 15- | Homo sapiens |
| | | | | hydroxyprostaglandin | |
| | | | | dehydrogenase | |
| 130 | AAH00690 | 794 | 100 | BC000690 ribosomal protein L24 | Homo sapiens |
| 130 | BC002110 | 794 | 100 | ribosomal protein L24 | Mus musculus |
| 130 | X78443 | 794 | 100 | ribosomal protein L24 | Rattus |
| | | | | | norvegicus |
| 131 | AAH11402 | 1122 | 100 | BC011402 Similar to decidual | Homo sapiens |
| | | | | protein induced by progesterone | |
| 131 | AB022718 | 1122 | 100 | DEPP | Homo sapiens |
| 131 | AL136653 | 1116 | 99 | hypothetical protein | Homo sapiens |
| 132 | AF108841 | 402 | 81 | pol protein | Homo sapiens |
| 132 | AF108843 | 415 | 75 | pol protein | Homo sapiens |
| 132 | U88902 | 317 | 73 | integrase | Homo sapiens |
| 133 | A07358 | 1408 | 99 | VAC beta | Mus musculus |
| 133 | AAH04376 | 1417 | 100 | BC004376 annexin A8 | Homo sapiens |
| 133 | X16662 | 1408 | 99 | vascular anticoagulant-beta (AA 1-327) | Homo sapiens |
| 134 | AF068227 | 2236 | 100 | putative transmembrane protein | Homo sapiens |
| 134 | AK032293 | 1426 | 76 | unnamed protein product | Mus musculus |
| 134 | BC025487 | 1447 | 77 | Similar to ceroid-lipofuscinosis, | Mus musculus |
| | | | | neuronal 5 | |
| 135 | AAH10652 | 1355 | 100 | BC010652 Unknown (protein for | Homo sapiens |
| | | | | MGC: 9753) | |
| 135 | AF217980_1 | 1338 | 98 | AF217980 unknown | Homo sapiens |
| 135 | AX119063 | 965 | 96 | unnamed protein product | Homo sapiens |
| 136 | AK000496 | 374 | 71 | unnamed protein product | Homo sapiens |
| 136 | AK025116 | 400 | 72 | unnamed protein product | Homo sapiens |
| 136 | AK057830 | 369 | 72 | unnamed protein product | Homo sapiens |
| 137 | AF001900 | 1702 | 100 | secreted frizzled-related protein | Homo sapiens |
| 137 | AF056087 | 1702 | 100 | secreted frizzled related protein | Homo sapiens |
| 137 | BC036503 | 1690 | 99 | Unknown (protein for MGC: 43294) | Homo sapiens |
| 138 | AAH14052 | 2127 | 100 | BC014052 transcriptional co- | Homo sapiens |
| | | | | activator with PDZ-binding motif | |
| | | | | (TAZ) | |
| 138 | AJ299431 | 2127 | 100 | transcriptional coactivator | Homo sapiens |
| 138 | AK022036 | 2127 | 100 | unnamed protein product | Homo sapiens |
| 139 | AAH07493 | 254 | 80 | BC007493 galactosidase, beta 1 | Homo sapiens |
| 139 | M22590 | 254 | 80 | beta-galactosidase precursor (EC | Homo sapiens |
| | | | | 3.2.1.23) | |
| 139 | M34423 | 254 | 80 | beta-galactosidase precursor (EC | Homo sapiens |
| | | | | 3.2.1.23) | |
| 140 | AAH15928 | 2186 | 100 | BC015928 Unknown (protein for | Homo sapiens |
| | | | | MGC: 8773) | |
| 140 | AB037797 | 2313 | 100 | KIAA1376 protein | Homo sapiens |
| 140 | AK088288 | 2153 | 98 | unnamed protein product | Mus musculus |
| 141 | AF087873_1 | 383 | 100 | AF087873 protein kinase inhibitor | Homo sapiens |
| 141 | AF225513_1 | 383 | 100 | AF225513 cAMP-dependent protein | Homo sapiens |
| | | | | kinase inhibitor beta | |
| 141 | BC036011 | 383 | 100 | protein kinase (cAMP-dependent, | Homo sapiens |
| | | | | catalytic) inhibitor beta | |
| 142 | X53305 | 736 | 100 | stathmin | Homo sapiens |
| 142 | X94912 | 736 | 100 | Pr22 | Homo sapiens |
| 142 | Z11566 | 736 | 100 | Pr22 protein | Homo sapiens |
| 143 | AF148856_2 | 150 | 79 | AF148856 unknown | Homo sapiens |
| 143 | AL096744 | 153 | 82 | hypothetical protein | Homo sapiens |
| 143 | M80341 | 150 | 79 | ORF2 | Homo sapiens |
| 144 | AF421375 | 619 | 44 | unknown | Homo sapiens |
| 144 | AK002154 | 798 | 94 | unnamed protein product | Homo sapiens |
| 144 | AK023417 | 819 | 100 | unnamed protein product | Homo sapiens |
| 145 | AF151807_1 | 2197 | 99 | AF151807 CGI-49 protein | Homo sapiens |
| 145 | AK075178 | 2150 | 97 | unnamed protein product | Homo sapiens |
| 145 | BC026185 | 2137 | 97 | Similar to CGI-49 protein | Homo sapiens |
| 146 | AAH10123 | 2134 | 100 | BC010123 SH3-domain binding | Homo sapiens |
| | | | | protein 5 (BTK-associated) | |
| 146 | AB005047 | 2134 | 100 | SH3 binding protein | Homo sapiens |
| 146 | AB027562 | 2008 | 94 | vascular endothelial cell | Rattus |
| | | | | specific protein 18 | norvegicus |
| 147 | AJ131023 | 5309 | 99 | oxytocinase/insulin-responsive | Homo sapiens |
| | | | | aminopeptidase, variant 1 | |
| 147 | U62768 | 5318 | 99 | oxytocinase splice variant 1 | Homo sapiens |
| 147 | U62769 | 5242 | 99 | oxytocinase splice variant 2 | Homo sapiens |
| 148 | AF234263 | 2521 | 99 | cathepsin C | Homo sapiens |
| 148 | U79415 | 2527 | 100 | prepro dipeptidyl peptidase I | Homo sapiens |
| 148 | X87212 | 2527 | 100 | cathepsin C | Homo sapiens |
| 149 | BC003426 | 1438 | 94 | Similar to eukaryotic translation | Mus musculus |
| | | | | initiation factor 2B, subunit 1 | |
| | | | | (alpha, 26 kD) | |
| 149 | U05821 | 1419 | 92 | translation initiation factor | Rattus |
| | | | | eIF-2B alpha-subunit | norvegicus |
| 149 | X95648 | 1526 | 100 | alfa subunit | Homo sapiens |
| 150 | AAH14093 | 2678 | 97 | BC014093 Similar to amiloride | Homo sapiens |
| | | | | binding protein 1 (amine oxidase | |
| | | | | (copper-containing)) | |
| 150 | M55602 | 2431 | 89 | amiloride-binding protein | Homo sapiens |
| 150 | X78212 | 2678 | 97 | amiloride binding protein | Homo sapiens |
| 151 | AK003633 | 925 | 98 | unnamed protein product | Mus musculus |
| 151 | AK007989 | 925 | 98 | unnamed protein product | Mus musculus |
| 151 | AK076212 | 925 | 98 | unnamed protein product | Mus musculus |
| 152 | AAH18340 | 1692 | 99 | BC018340 Unknown (protein for | Homo sapiens |
| | | | | MGC: 9372) | |
| 152 | X79066 | 1692 | 99 | ERF-1 | Homo sapiens |
| 152 | X99404 | 1684 | 99 | Berg36 | Homo sapiens |
| 153 | AB055977 | 492 | 93 | I3 protein | Homo sapiens |
| 153 | AF041430 | 492 | 93 | pRGR2 | Homo sapiens |
| 153 | AF106966 | 492 | 93 | I3 protein | Homo sapiens |
| 154 | AAH14438 | 1018 | 100 | BC014438 Unknown (protein for | Homo sapiens |
| | | | | MGC: 22947) | |
| 154 | AE006639_3 | 1018 | 100 | AE006639 HN1 like | Homo sapiens |
| 154 | AK023154 | 1018 | 100 | unnamed protein product | Homo sapiens |
| 155 | J03779 | 3944 | 100 | common acute lymphoblastic | Homo sapiens |
| | | | | leukemia antigen precursor | |
| 155 | M26628 | 3919 | 99 | enkephalinase | Homo sapiens |
| 155 | Y00811 | 3919 | 99 | CALLA protein (AA 1-750) | Homo sapiens |
| 156 | AB035304 | 4092 | 100 | cadherin-5 | Homo sapiens |
| 156 | U84722 | 4086 | 99 | vascular endothelial cadherin | Homo sapiens |
| 156 | X79981 | 4092 | 100 | VE-cadherin | Homo sapiens |
| 157 | AX019983 | 739 | 100 | unnamed protein product | Homo sapiens |
| 157 | BC026254 | 739 | 100 | insulin-like 4 (placenta) | Homo sapiens |
| 157 | L34838 | 739 | 100 | EPIL | Homo sapiens |
| 158 | AF139894_1 | 1813 | 100 | AF139894 RNA-binding protein | Mus musculus |
| | | | | alpha-CP1 | |
| 158 | AF139895_1 | 1813 | 100 | AF139895 RNA-binding protein | Mus musculus |
| | | | | alpha-CP1 | |
| 158 | BC004793 | 1813 | 100 | poly(rC)-binding protein 1 | Mus musculus |
| 159 | U12390 | 369 | 67 | beta-galactosidase alpha peptide | Cloning |
| | | | | | vector |
| | | | | | pSport1 |
| 159 | U12391 | 355 | 88 | beta-galactosidase alpha peptide | Cloning |
| | | | | | vector |
| | | | | | pSport2 |
| 159 | Y14836 | 294 | 68 | beta-galactosidase | Phagemid |
| | | | | | cloning |
| | | | | | vector |
| | | | | | pTZ19U |
| 160 | AF218012_1 | 1751 | 100 | AF218012 unknown | Homo sapiens |
| 160 | AF218030_1 | 1751 | 100 | AF218030 unknown | Homo sapiens |
| 160 | AL121586 | 1778 | 100 | dJ477O4.1.1 (novel protein | Homo sapiens |
| | | | | similar to otoferlin and | |
| | | | | dysferlin, isoform 1) | |
| 161 | AAH00352 | 1286 | 100 | BC000352 proteasome (prosome, | Homo sapiens |
| | | | | macropain) activator subunit 1 | |
| | | | | (PA28 alpha) | |
| 161 | AAH07503 | 1286 | 100 | BC007503 proteasome (prosome, | Homo sapiens |
| | | | | macropain) activator subunit 1 | |
| | | | | (PA28 alpha) | |
| 161 | AF078829_1 | 1286 | 100 | AF078829 proteasome activator | Homo sapiens |
| | | | | PA28 alpha | |
| 162 | AAH16155 | 382 | 100 | BC016155 Ras homolog enriched in | Homo sapiens |
| | | | | brain 2 | |
| 162 | AF493921_1 | 382 | 100 | AF493921 Ras family small GTP | Homo sapiens |
| | | | | binding protein RHEB2 | |
| 162 | Z29677 | 382 | 100 | Ras-related GTP-binding protein | Homo sapiens |
| 163 | AAH10423 | 2657 | 100 | BC010423 Ig superfamily receptor | Homo sapiens |
| | | | | LNIR | |
| 163 | AF160477_1 | 2655 | 99 | AF160477 Ig superfamily receptor | Homo sapiens |
| | | | | LNIR precursor | |
| 163 | AF426163_1 | 2657 | 100 | AF426163 nectin 4 | Homo sapiens |
| 164 | AJ295142 | 3578 | 99 | Clq receptor protein | Homo sapiens |
| 164 | BC028075 | 3577 | 99 | complement component 1, q | Homo sapiens |
| | | | | subcomponent, receptor 1 | |
| 164 | U94333 | 3587 | 100 | C1qR(p) | Homo sapiens |
| 165 | AAH14271 | 3392 | 100 | BC014271 endoglin (Osler-Rendu- | Homo sapiens |
| | | | | Weber syndrome 1) | |
| 165 | J05481 | 3319 | 99 | endoglin precursor | Homo sapiens |
| 165 | U37439 | 3385 | 99 | endoglin | Homo sapiens |
| 166 | AAH07411 | 323 | 98 | BC007411 Unknown (protein for | Homo sapiens |
| | | | | MGC: 2554) | |
| 166 | AF051782 | 319 | 97 | diaphanous 1 | Homo sapiens |
| 166 | U96963 | 322 | 98 | p140mDia | Mus musculus |
| 167 | AF151966_1 | 1875 | 66 | AF151966 GTPase activating | Gallus |
| | | | | protein Rap1-GAP | gallus |
| 167 | BC030891 | 2109 | 95 | Unknown (protein for | Mus musculus |
| | | | | IMAGE: 4485689) | |
| 167 | M64788 | 3477 | 100 | GTPase activating protein | Homo sapiens |
| 168 | AX206708 | 1968 | 100 | unnamed protein product | Homo sapiens |
| 168 | M92934 | 1968 | 100 | connective tissue growth factor | Homo sapiens |
| 168 | X78947 | 1968 | 100 | connective tissue growth factor | Homo sapiens |
| 169 | AAH18823 | 849 | 100 | BC018823 Unknown (protein for | Homo sapiens |
| | | | | MGC: 1236) | |
| 169 | AK007729 | 809 | 97 | unnamed protein product | Mus musculus |
| 169 | U30826 | 849 | 100 | SRp40-1 | Homo sapiens |
| 170 | AAH07333 | 2823 | 100 | BC007333 etc variant gene 5 (ets- | Homo sapiens |
| | | | | related molecule) | |
| 170 | X76184 | 2823 | 100 | etc-related protein | Homo sapiens |
| 170 | X96381 | 2823 | 100 | erm | Homo sapiens |
| 171 | AAH02599 | 996 | 99 | BC002599 corticotropin releasing | Homo sapiens |
| | | | | hormone | |
| 171 | AAH11031 | 996 | 99 | BC011031 Similar to corticotropin | Homo sapiens |
| | | | | releasing hormone | |
| 171 | V00571 | 996 | 99 | corticotropin releasing factor | Homo sapiens |
| 172 | AF151872_1 | 1069 | 100 | AF151872 CGI-114 protein | Homo sapiens |
| 172 | AL110239 | 1066 | 99 | hypothetical protein | Homo sapiens |
| 172 | AX399865 | 1069 | 100 | unnamed protein product | Homo sapiens |
| 173 | AAH00732 | 1553 | 100 | BC000732 glioblastoma amplified | Homo sapiens |
| | | | | sequence | |
| 173 | AAH01837 | 1553 | 100 | BC001837 glioblastoma amplified | Homo sapiens |
| | | | | sequence | |
| 173 | BC030821 | 1553 | 100 | glioblastoma amplified sequence | Homo sapiens |
| 174 | AAH01483 | 2103 | 100 | BC001483 Unknown (protein for | Homo sapiens |
| | | | | MGC: 1364) | |
| 174 | AAH09241 | 2103 | 100 | BC009241 Unknown (protein for | Homo sapiens |
| | | | | MGC: 3556) | |
| 174 | BC023599 | 2103 | 100 | TRK-fused gene | Homo sapiens |
| 175 | AAH12806 | 3278 | 100 | BC012806 solute carrier family 5 | Homo sapiens |
| | | | | (sodium-dependent vitamin | |
| | | | | transporter), member 6 | |
| 175 | AAH15631 | 3278 | 100 | BC015631 solute carrier family 5 | Homo sapiens |
| | | | | (sodium-dependent vitamin | |
| | | | | transporter), member 6 | |
| 175 | AF116241 | 3272 | 99 | sodium-dependent multivitamin | Homo sapiens |
| | | | | transporter | |
| 177 | AAH04324 | 744 | 100 | BC004324 ribosomal protein S16 | Homo sapiens |
| 177 | AAH07977 | 744 | 100 | BC007977 ribosomal protein S16 | Homo sapiens |
| 177 | X17665 | 744 | 100 | ribosomal protein S16 (AA 1-146) | Rattus |
| | | | | | rattus |
| 178 | AB047555 | 624 | 90 | IZP6 | Mus musculus |
| | | | | | musculus |
| 178 | AK007948 | 624 | 90 | unnamed protein product | Mus musculus |
| 178 | BC004729 | 624 | 90 | RIKEN cDNA 1810063P04 gene | Mus musculus |
| 179 | BC003337 | 1957 | 100 | actin, gamma 1 | Mus musculus |
| 179 | BC021796 | 1957 | 100 | actin, gamma 1 | Mus musculus |
| 179 | BC023248 | 1957 | 100 | actin, gamma, cytoplasmic | Mus musculus |
| 180 | AAH11700 | 2048 | 100 | BC011700 glutamate-ammonia ligase | Homo sapiens |
| | | | | (glutamine synthase) | |
| 180 | AAH11852 | 2048 | 100 | BC011852 glutamate-ammonia ligase | Homo sapiens |
| | | | | (glutamine synthase) | |
| 180 | AAH18992 | 2048 | 100 | BC018992 Unknown (protein for | Homo sapiens |
| | | | | MGC: 20095 | |
| 181 | AF234654_1 | 1155 | 100 | AF234654 PLAC1 | Homo sapiens |
| 181 | AK075086 | 1155 | 100 | unnamed protein product | Homo sapiens |
| 181 | BC022335 | 1155 | 100 | placenta-specific 1 | Homo sapiens |
| 182 | AAB67977 | 4108 | 99 | AF019413 complement factor B | Homo sapiens |
| 182 | AAH04143 | 4121 | 100 | BC004143 B-factor, properdin | Homo sapiens |
| 182 | AAH07990 | 4121 | 100 | BC007990 B-factor, properdin | Homo sapiens |
| 183 | AAH17399 | 355 | 98 | BC017399 Similar to RIKEN cDNA | Homo sapiens |
| | | | | 1810037I17 gene | |
| 183 | AK007721 | 236 | 68 | unnamed protein product | Mus musculus |
| 183 | BC002135 | 236 | 68 | Unknown (protein for MGC: 7243) | Mus musculus |
| 184 | AAH13383 | 1701 | 100 | BC013383 calumenin | Homo sapiens |
| 184 | AF013759 | 1701 | 100 | calumein | Homo sapiens |
| 184 | U67280 | 1695 | 99 | calumenin | Homo sapiens |
| 185 | AF069065 | 596 | 97 | induced cAMP early repressor | Homo sapiens |
| | | | | type 1 | |
| 185 | AJ311667 | 588 | 95 | induced cAMP early repressor, | Mus musculus |
| | | | | ICER I | |
| 185 | D14826 | 608 | 100 | hCREM 2alpha-a protein | Homo sapiens |
| 186 | AAH00452 | 1043 | 100 | BC000452 Similar to thioredoxin | Homo sapiens |
| | | | | peroxidase 1 | |
| 186 | AAH03022 | 1043 | 100 | BC003022 Similar to thioredoxin | Homo sapiens |
| | | | | peroxidase 1 | |
| 186 | BC039428 | 1043 | 100 | Similar to peroxiredoxin 2 | Homo sapiens |
| 187 | AB056107 | 2995 | 100 | zinc finger protein homologous to | Homo sapiens |
| | | | | mouse Zfp91 | |
| 187 | AB057443 | 2775 | 100 | testis specific ZFP91 | Homo sapiens |
| 187 | AF310246_1 | 2586 | 99 | AF310246 FKSG11 | Homo sapiens |
| 188 | AF023476 | 4954 | 100 | meltrin-L precursor | Homo sapiens |
| 188 | AF023477 | 3855 | 99 | meltrin-S | Homo sapiens |
| 188 | D50411 | 3939 | 80 | meltrin alpha | Mus musculus |
| 189 | AF154121_1 | 2195 | 94 | AF154121 sodium-dependent high- | Homo sapiens |
| | | | | affinity dicarboxylate | |
| | | | | transporter | |
| 189 | AL034424 | 2126 | 91 | dJ257E24.2.1 (sodium-dependent | Homo sapiens |
| | | | | high-affinity dicarboxylate | |
| | | | | transporter (NADC3, SDCT2) | |
| | | | | (isoform 1)) | |
| 189 | AY072810 | 2126 | 91 | energy metabolism-related sodium- | Homo sapiens |
| | | | | dependent high-affinity | |
| | | | | dicarboxylate transporter | |
| 190 | AF400594 | 352 | 54 | endothelial nitric oxide synthase | Homo sapiens |
| 190 | AF519768_1 | 352 | 54 | AF519768 nitric oxide synthase 3 | Homo sapiens |
| | | | | (endothelial cell) | |
| 190 | D26607 | 350 | 53 | endothelial nitric oxide synthase | Homo sapiens |
| 191 | AAH01462 | 1835 | 100 | BC001462 protease, serine, 8 | Homo sapiens |
| | | | | (prostasin) | |
| 191 | L41351 | 1835 | 100 | prostasin | Homo sapiens |
| 191 | U33446 | 1835 | 100 | prostasin | Homo sapiens |
| 192 | AK025116 | 272 | 73 | unnamed protein product | Homo sapiens |
| 192 | AK090913 | 310 | 71 | unnamed protein product | Homo sapiens |
| 192 | BC033959 | 200 | 70 | similar to Alu subfamily SQ | Homo sapiens |
| | | | | sequence contamination warning | |
| | | | | entry | |
| 193 | AF346465 | 7291 | 91 | latent transforming growth factor | Mus musculus |
| | | | | beta binding protein 1S | |
| 193 | AF489528_1 | 7887 | 99 | AF489528 transforming growth | Homo sapiens |
| | | | | factor-beta binding protein-1S | |
| 193 | M34057 | 7903 | 100 | transforming growth factor-beta 1 | Homo sapiens |
| | | | | binding protein precursor | |
| 194 | AY004254 | 1199 | 90 | endoplasmic reticulum protein 29 | Rattus |
| | | | | precursor | norvegicus |
| 194 | X94910 | 1334 | 100 | ERp28 | Homo sapiens |
| 194 | Y10264 | 1199 | 90 | ERp29 precursor | Rattus |
| | | | | | norvegicus |
| 195 | AF119917_58 | 250 | 100 | AF119896 PRO2751 | Homo sapiens |
| 196 | AB020692 | 4158 | 100 | KIAA0885 protein | Homo sapiens |
| 196 | BC016898 | 4107 | 98 | Unknown (protein for MGC: 19174) | Mus musculus |
| 196 | X52311 | 4102 | 98 | unr protein (AA 1-798) | Rattus |
| | | | | | norvegicus |
| 198 | AL049698 | 7034 | 98 | dJ470B24.1.3 (myeloid/lymphoid or | Homo sapiens |
| | | | | mixed-lineage leukemia (trithorax | |
| | | | | (Drosophila) homolog); | |
| | | | | translocated to, 4 (AF-6) | |
| | | | | (isoform 3)) | |
| 198 | AL049698 | 7050 | 98 | dJ470B24.1.5 (myeloid/lymphoid or | Homo sapiens |
| | | | | mixed-lineage leukemia (trithorax | |
| | | | | (Drosophila) homolog); | |
| | | | | translocated to, 4 (AF-6) | |
| | | | | (isoform 5)) | |
| 198 | AL161973 | 7363 | 100 | hypothetical protein | Homo sapiens |
| 199 | AAH21288 | 2134 | 100 | BC021288 Unknown (protein for | Homo sapiens |
| | | | | MGC: 29494) | |
| 199 | J03263 | 1934 | 98 | membrane glycoprotein | Homo sapiens |
| 199 | J04182 | 2041 | 96 | lysosomal membrane glycoprotein-1 | Homo sapiens |
| 200 | AC002400 | 817 | 100 | Acyl carrier protein, | Homo sapiens |
| | | | | Mitochondrial (ACP) (5′partial) | |
| 200 | AF087660_1 | 788 | 100 | AF087660 NADH: ubiquinone | Homo sapiens |
| | | | | oxidoreductase SDAP subunit | |
| 200 | AK018717 | 672 | 85 | unnamed protein product | Mus musculus |
| 201 | AB031550 | 1298 | 87 | pctp-L | Mus musculus |
| 201 | AF151810_1 | 1470 | 90 | AF151810 CGI-52 protein | Homo sapiens |
| 201 | Z81134 | 419 | 39 | Hypothetical protein T28D6.7 | Caenorhabditis |
| | | | | | elegans |
| 203 | AAH09392 | 5306 | 99 | BC009392 Unknown (protein for | Homo sapiens |
| | | | | MGC: 15329) | |
| 203 | AB011149 | 5318 | 100 | KIAA0577 protein | Homo sapiens |
| 203 | AP000512 | 5318 | 100 | RNA helicase | Homo sapiens |
| 204 | AAH08751 | 3757 | 99 | BC008751 calpain 1, (mu/I) large | Homo sapiens |
| | | | | subunit | |
| 204 | AAH17200 | 3762 | 100 | BC017200 calpain 1, (mu/I) large | Homo sapiens |
| | | | | subunit | |
| 204 | X04366 | 3762 | 100 | CANP, large subunit (aa 1-714) | Homo sapiens |
| 205 | AB093679 | 740 | 100 | ribosomal protein L26 | Macaca |
| | | | | | fascicularis |
| 205 | X69392 | 740 | 100 | ribosomal protein L26 | Homo sapiens |
| 205 | X80699 | 740 | 100 | L26 | Mus musculus |
| 206 | AAH06391 | 1974 | 100 | BC006391 inhibin, alpha | Homo sapiens |
| 206 | M13981 | 1974 | 100 | inhibin A-subunit precursor | Homo sapiens |
| 206 | X04445 | 1974 | 100 | preproinhibin | Homo sapiens |
| 207 | AK025116 | 190 | 66 | unnamed protein product | Homo sapiens |
| 207 | AK092450 | 300 | 63 | unnamed protein product | Homo sapiens |
| 207 | AK097214 | 305 | 68 | unnamed protein product | Homo sapiens |
| 208 | AF130117_48 | 337 | 73 | AF130089 PRO2550 | Homo sapiens |
| 208 | AK000385 | 333 | 70 | unnamed protein product | Homo sapiens |
| 208 | AK092450 | 336 | 70 | unnamed protein product | Homo sapiens |
| 209 | AB062485 | 161 | 77 | OK/SW-CL.18 | Homo sapiens |
| 209 | AF090930_1 | 172 | 51 | AF090930 PRO0478 | Homo sapiens |
| 209 | AX399919 | 158 | 68 | unnamed protein product | Homo sapiens |
| 210 | AF063591_1 | 1401 | 99 | AF063591 brain my033 protein | Homo sapiens |
| 210 | BC022522 | 1411 | 100 | antigen identified by monoclonal | Homo sapiens |
| | | | | antibody MRC OX-2 | |
| 210 | BC031103 | 1411 | 100 | antigen identified by monoclonal | Homo sapiens |
| | | | | antibody MRC OX-2 | |
| 211 | AAH00692 | 2582 | 100 | BC000692 Similar to | Homo sapiens |
| | | | | hyaluronoglucosaminidase 2 | |
| 211 | AC002455 | 2579 | 99 | human PH-20 homolog (LUCA-2) | Homo sapiens |
| 211 | U09577 | 2579 | 99 | lysosomal hyaluronidase | Homo sapiens |
| 212 | AAH02700 | 2294 | 100 | BC002700 Similar to keratin 7 | Homo sapiens |
| 212 | AF509887 | 2294 | 100 | keratin 7 | Homo sapiens |
| 212 | AF509892 | 2294 | 100 | keratin 7 | Homo sapiens |
| 213 | AF523361_1 | 1951 | 99 | AF523361 CD34 antigen | Homo sapiens |
| 213 | M81945 | 1955 | 100 | hematopoietic stem cell antigen | Homo sapiens |
| 213 | S53910 | 1951 | 99 | CD34 | Homo sapiens |
| 214 | AB037891 | 493 | 100 | Bc10 | Mus musculus |
| 214 | AF303656_1 | 493 | 100 | AF303656 bladder cancer-related | Mus musculus |
| | | | | protein | |
| 214 | BC026935 | 493 | 100 | bladder cancer associated protein | Mus musculus |
| 215 | AF322909 | 3026 | 100 | transmembrane glycoprotein HGFIN | Homo sapiens |
| 215 | BC032783 | 3003 | 97 | Similar to glycoprotein | Homo sapiens |
| | | | | (transmembrane) nmb | |
| 215 | X76534 | 3026 | 100 | NMB | Homo sapiens |
| 216 | AAH17352 | 3091 | 100 | BC017352 Unknown (protein for | Homo sapiens |
| | | | | MGC: 29525) | |
| 216 | AF230388_1 | 3085 | 99 | AF230388 tripartite motif protein | Homo sapiens |
| | | | | TRIM29 alpha | |
| 216 | L24203 | 3085 | 99 | ataxia-telangiectasia group D- | Homo sapiens |
| | | | | associated protein | |
| 217 | AK003609 | 523 | 100 | unnamed protein product | Mus musculus |
| 217 | AL136635 | 523 | 100 | hypothetical protein | Homo sapiens |
| 217 | BC016408 | 523 | 100 | RIKEN cDNA 1110011F09 gene | Mus musculus |
| 218 | AF073310 | 7103 | 100 | insulin receptor substrate-2 | Homo sapiens |
| 218 | AF385932_1 | 7014 | 99 | AF385932 insulin receptor | Homo sapiens |
| | | | | substrate 2 insertion mutant | |
| 218 | AF385933_1 | 7026 | 99 | AF385933 insulin receptor | Homo sapiens |
| | | | | substrate 2 insertion mutant | |
| 219 | AAH09586 | 1584 | 70 | BC009586 Unknown (protein for | Homo sapiens |
| | | | | MGC: 14649) | |
| 219 | D26485 | 1584 | 70 | core I protein | Homo sapiens |
| 219 | L16842 | 1584 | 70 | ubiquinol-cytochrome c reductase | Homo sapiens |
| | | | | core I protein | |
| 220 | AF062534 | 1919 | 100 | genethonin 1 | Homo sapiens |
| 220 | AK050416 | 1083 | 62 | unnamed protein product | Mus musculus |
| 220 | BC022301 | 1919 | 100 | genethonin 1 | Homo sapiens |
| 221 | AAH08950 | 956 | 100 | BC008950 Unknown (protein for | Homo sapiens |
| | | | | MGC: 3593) | |
| 221 | AF112202_1 | 956 | 100 | AF112202 prenylated rab acceptor 1 | Homo sapiens |
| 221 | AJ133534 | 951 | 99 | prenylated Rab acceptor 1 (PRA1) | Homo sapiens |
| 222 | AAH14257 | 888 | 100 | BC014257 Similar to DKFZp564J157 | Homo sapiens |
| | | | | protein | |
| 222 | AAH16064 | 888 | 100 | BC016064 DKFZp564J157 protein | Homo sapiens |
| 222 | AX405840 | 888 | 100 | unnamed protein product | Homo sapiens |
| 223 | AAH02394 | 2275 | 100 | BC002394 ceroid-lipofuscinosis, | Homo sapiens |
| | | | | neuronal 3, juvenile (Batten, | |
| | | | | Spielmeyer-Vogt disease) | |
| 223 | AAH04433 | 2275 | 100 | BC004433 ceroid-lipofuscinosis, | Homo sapiens |
| | | | | neuronal 3, juvenile (Batten, | |
| | | | | Spielmeyer-Vogt disease) | |
| 223 | U32680 | 2275 | 100 | CLN3 protein | Homo sapiens |
| 224 | AF443072_1 | 17656 | 99 | AF443072 laminin alpha5 chain | Homo sapiens |
| | | | | precursor | |
| 224 | AL354836 | 17668 | 99 | bA157P1.1.1 (laminin alpha 5) | Homo sapiens |
| 224 | AX463738 | 17656 | 99 | laminin 5 | Homo sapiens |
| 225 | BC024366 | 703 | 100 | ribosomal protein L27 | Mus musculus |
| 225 | X07424 | 703 | 100 | ribosomal protein L27 (AA 1-136) | Rattus |
| | | | | | norvegicus |
| 225 | X56852 | 703 | 100 | ribosomal protein L27 | Gallus |
| | | | | | gallus |
| 226 | AAH13131 | 969 | 100 | BC013131 Similar to hypothetical | Homo sapiens |
| | | | | protein FLJ10702 | |
| 226 | AK001564 | 969 | 100 | unnamed protein product | Homo sapiens |
| 226 | BC013719 | 969 | 100 | hypothetical protein FLJ10702 | Mus musculus |
| 227 | AF214737_1 | 5676 | 100 | AF214737 C9orf10a | Homo sapiens |
| 227 | BC010304 | 3765 | 93 | Unknown (protein for | Mus musculus |
| | | | | IMAGE: 3597827) | |
| 227 | D80005 | 5676 | 100 | KIAA0183 | Homo sapiens |
| 228 | AF113127_1 | 1186 | 99 | AF113127 S1R protein | Homo sapiens |
| 228 | AF151877_1 | 1203 | 94 | AF151877 CGI-119 protein | Homo sapiens |
| 228 | AF182041_1 | 1186 | 99 | AF182041 z-protein | Homo sapiens |
| 229 | AAH11616 | 1514 | 100 | BC011616 Similar to cyclin D3 | Homo sapiens |
| 229 | AF517525_1 | 1511 | 99 | AF517525 cyclin D3 | Homo sapiens |
| 229 | M92287 | 1514 | 100 | cyclin D3 | Homo sapiens |
| 230 | AAH11581 | 4280 | 100 | BC011581 Similar to oxysterol | Homo sapiens |
| | | | | binding protein | |
| 230 | AF185696_1 | 4280 | 100 | AF185696 oxysterol-binding | Homo sapiens |
| | | | | protein 1 | |
| 230 | AF185705 | 4280 | 100 | oxysterol binding protein 1 | Homo sapiens |
| 231 | AF053745 | 462 | 32 | pol polyprotein | Mus dunni |
| | | | | | endogenous |
| | | | | | virus |
| 231 | AF151794 | 473 | 35 | pol protein | Phascolarctos |
| | | | | | cinereus |
| 231 | D10032 | 506 | 36 | gag-pol precursor polyprotein | Baboon |
| | | | | | endogenous |
| | | | | | virus strain |
| | | | | | M7 |
| 232 | AF012920 | 625 | 100 | GEC-1 | Cavia |
| | | | | | porcellus |
| 232 | AF312680 | 625 | 100 | GEC-1 | Cavia |
| | | | | | porcellus |
| 232 | BC004602 | 625 | 100 | RIKEN cDNA 9130422N19 gene | Mus musculus |
| 233 | AAH14241 | 1733 | 100 | BC014241 Similar to hypothetical | Homo sapiens |
| | | | | protein, MGC: 7035 | |
| 233 | AB093636 | 1687 | 96 | hypothetical protein | Macaca |
| | | | | | fascicularis |
| 233 | AK046512 | 1274 | 74 | unnamed protein product | Mus musculus |
| 234 | AF140240_1 | 749 | 100 | AF140240 transcription factor | Homo sapiens |
| | | | | TBX3 | |
| 234 | AK075457 | 749 | 100 | unnamed protein product | Homo sapiens |
| 234 | BC025258 | 749 | 100 | T-box 3 (ulnar mammary syndrome) | Homo sapiens |
| 235 | AB062477 | 166 | 64 | OK/SW-CL.41 | Homo sapiens |
| 235 | AK054840 | 167 | 65 | unnamed protein product | Homo sapiens |
| 235 | AK092888 | 224 | 74 | unnamed protein product | Homo sapiens |
| 236 | AAH01863 | 320 | 100 | BC001863 hypoxia-inducible | Homo sapiens |
| | | | | protein 2 | |
| 236 | AF144755_1 | 320 | 100 | AF144755 hypoxia-inducible | Homo sapiens |
| | | | | protein 2 | |
| 236 | AX105363 | 320 | 100 | unnamed protein product | Homo sapiens |
| 237 | AAH00718 | 1025 | 97 | BC000718 Similar to dynamitin | Homo sapiens |
| | | | | (dynactin complex 50 kD subunit) | |
| 237 | AAH09468 | 1025 | 97 | BC009468 dynactin 2 (p50) | Homo sapiens |
| 237 | AAH14083 | 1025 | 97 | BC014083 dynactin 2 (p50) | Homo sapiens |
| 238 | AB037669 | 2790 | 100 | L-type amino acid transporter 2 | Homo sapiens |
| 238 | AF171669_1 | 2790 | 100 | AF171669 glycoprotein-associated | Homo sapiens |
| | | | | amino acid transporter LAT2 | |
| 238 | Y18483 | 2778 | 99 | SLC7A8 protein | Homo sapiens |
| 239 | AF118094_21 | 187 | 48 | AF118082 PRO1902 | Homo sapiens |
| 239 | AF119917_13 | 158 | 61 | AF119851 PRO1722 | Homo sapiens |
| 239 | AK097266 | 185 | 60 | unnamed protein product | Homo sapiens |
| 240 | AK075392 | 1353 | 90 | unnamed protein product | Homo sapiens |
| 240 | AX136157 | 1353 | 90 | unnamed protein product | Homo sapiens |
| 240 | AX179293 | 1356 | 90 | 21676 ADH | Homo sapiens |
| 241 | AF328769_1 | 2426 | 91 | AF328769 PC2-glutamine-rich- | Homo sapiens |
| | | | | associated protein | |
| 241 | AK090465 | 2677 | 99 | FLJ00386 protein | Homo sapiens |
| 241 | BC017110 | 2677 | 99 | Unknown (protein for MGC: 16010) | Homo sapiens |
| 242 | AAH10514 | 2363 | 100 | BC010514 clusterin (complement | Homo sapiens |
| | | | | lysis inhibitor, SP-40,40, | |
| | | | | sulfated glycoprotein 2, | |
| | | | | testosterone-repressed prostate | |
| | | | | message 2, apolipoprotein J) | |
| 242 | AAH19588 | 2363 | 100 | BC019588 clusterin (complement | Homo sapiens |
| | | | | lysis inhibitor, SP-40,40, | |
| | | | | sulfated glycoprotein 2, | |
| | | | | testosterone-repressed prostate | |
| | | | | message 2, apolipoprotein J) | |
| 242 | X14723 | 2363 | 100 | SP-40,40 prepropetide (AA −22 to | Homo sapiens |
| | | | | 427) | |
| 243 | AAH06786 | 1161 | 100 | BC006786 cellular repressor of | Homo sapiens |
| | | | | E1A-stimulated genes | |
| 243 | AAH08628 | 1161 | 100 | BC008628 cellular repressor of | Homo sapiens |
| | | | | E1A-stimulated genes | |
| 243 | AF084523 | 1161 | 100 | cellular repressor of E1A- | Homo sapiens |
| | | | | stimulated genes CREG | |
| 244 | AX167114 | 8220 | 99 | unnamed protein product | Homo sapiens |
| 244 | BC036531 | 8206 | 99 | Unknown (protein for MGC: 33668) | Homo sapiens |
| 244 | Z74615 | 8220 | 99 | prepro-alpha1(I) collagen | Homo sapiens |
| 245 | AB025412 | 5671 | 98 | Ten-m3 | Mus musculus |
| 245 | AF195418_1 | 5676 | 99 | AF195418 ODZ3 | Mus musculus |
| 245 | AK001336 | 5447 | 100 | unnamed protein product | Homo sapiens |
| 246 | AAH03550 | 1419 | 100 | BC003550 Similar to CGI-20 | Homo sapiens |
| | | | | protein | |
| 246 | AF132954_1 | 1577 | 99 | AF132954 CGI-20 protein | Homo sapiens |
| 246 | AK003551 | 1613 | 95 | unnamed protein product | Mus musculus |
| 247 | BC022512 | 242 | 96 | platelet/endothelial cell | Homo sapiens |
| | | | | adhesion molecule (CD31 antigen) | |
| 247 | L34657 | 242 | 96 | platelet endothelial cell | Homo sapiens |
| | | | | adhesion molecule-1 | |
| 247 | M28526 | 242 | 96 | PECAM-1 precursor | Homo sapiens |
| 248 | AAH12138 | 1849 | 100 | BC012138 Similar to guanine | Homo sapiens |
| | | | | nucleotide binding protein (G | |
| | | | | protein), alpha inhibiting | |
| | | | | activity polypeptide 2 | |
| 248 | AF493906_1 | 1849 | 100 | AF493906 guanine nucleotide | Homo sapiens |
| | | | | binding protein alpha i2 | |
| 248 | X04828 | 1849 | 100 | G protein alpha-subunit (AA 1-355) | Homo sapiens |
| 249 | AF081258_1 | 3091 | 100 | AF081258 testis-specific | Homo sapiens |
| | | | | chromodomain Y-like protein | |
| 249 | AF081259_1 | 3091 | 100 | AF081259 testis-specific | Homo sapiens |
| | | | | chromodomain Y-like protein | |
| 249 | AF081261_1 | 2614 | 88 | AF081261 testis-specific | Mus musculus |
| | | | | chromodomain Y-like protein | |
| 250 | AF162856_1 | 5147 | 65 | AF162856 receptor-like protein | Mus musculus |
| | | | | tyrosine phosphatase rho-1 | |
| 250 | X58287 | 7569 | 97 | protein-tyrosine phosphatase | Mus musculus |
| 250 | X58288 | 7731 | 99 | protein-tyrosine phosphatase | Homo sapiens |
| 251 | AK096998 | 303 | 65 | unnamed protein product | Homo sapiens |
| 251 | AK097965 | 304 | 73 | unnamed protein product | Homo sapiens |
| 251 | BC033959 | 333 | 77 | similar to Alu subfamily SQ | Homo sapiens |
| | | | | sequence contamination warning | |
| | | | | entry | |
| 252 | AAH18991 | 1272 | 100 | BC018991 hepatoma-derived growth | Homo sapiens |
| | | | | factor (high-mobility group | |
| | | | | protein 1-like) | |
| 252 | AJ237996 | 1205 | 95 | hepatoma derived growth factor | Bos taurus |
| 252 | D16431 | 1272 | 100 | hepatoma-derived GF | Homo sapiens |
| 253 | AAH17197 | 1785 | 99 | BC017197 myeloid cell leukemia | Homo sapiens |
| | | | | sequence 1 (BCL2-related) | |
| 253 | AF147742_1 | 1785 | 99 | AF147742 myeloid cell | Homo sapiens |
| | | | | differentiation protein | |
| 253 | AF162677 | 1785 | 99 | myeloid cell leukemia protein 1 | Homo sapiens |
| 254 | AF274892 | 2520 | 100 | glucose transporter 3 | Homo sapiens |
| 254 | BC039196 | 2520 | 100 | solute carrier family 2 | Homo sapiens |
| | | | | (facilitated glucose | |
| | | | | transporter), member 3 | |
| 254 | M20681 | 2520 | 100 | glucose transporter-like protein | Homo sapiens |
| 255 | AAH00730 | 3448 | 100 | BC000730 calpain 6 | Homo sapiens |
| 255 | AJ000388 | 3439 | 99 | calpain-like protease, CANPX | Homo sapiens |
| 255 | AL031117 | 3448 | 100 | dJ914P14.1 (calpain-like protease | Homo sapiens |
| | | | | CANPX) | |
| 256 | AAH03143 | 1980 | 100 | BC003143 dual specificity | Homo sapiens |
| | | | | phosphatase 6 | |
| 256 | AAH03562 | 1980 | 100 | BC003562 dual specificity | Homo sapiens |
| | | | | phosphatase 6 | |
| 256 | X93920 | 1980 | 100 | protein-tyrosine-phosphatase | Homo sapiens |
| 257 | AB023194 | 6006 | 100 | KIAA0977 protein | Homo sapiens |
| 257 | AK052533 | 3549 | 61 | unnamed protein product | Mus musculus |
| 257 | AL049939 | 1823 | 99 | hypothetical protein | Homo sapiens |
| 258 | AAH15031 | 4447 | 100 | BC015031 Unknown (protein for | Homo sapiens |
| | | | | MGC: 9105) | |
| 258 | AB018266 | 4447 | 100 | KIAA0723 protein | Homo sapiens |
| 258 | BC029070 | 4375 | 98 | Similar to matrin 3 | Mus musculus |
| 259 | AAH18726 | 1229 | 100 | BC018726 CD74 antigen (invariant | Homo sapiens |
| | | | | polypeptide of major | |
| | | | | histocompatibility complex, class | |
| | | | | II antigen-associated) | |
| 259 | M13560 | 1229 | 100 | cell surface glycoprotein | Homo sapiens |
| 259 | X00497 | 1229 | 100 | putative p33 | Homo sapiens |
| 260 | AF114262_3 | 2305 | 88 | AF114260 forkhead protein AFXH | Mus musculus |
| 260 | X93996 | 2624 | 100 | AFX | Homo sapiens |
| 260 | Y11284 | 2489 | 97 | AFX1 | Homo sapiens |
| 261 | AAH09409 | 1688 | 100 | BC009409 Unknown (protein for | Homo sapiens |
| | | | | MGC: 10655) | |
| 261 | X13425 | 1688 | 100 | GA733-1 protein (AA 1-323) | Homo sapiens |
| 261 | X77754 | 1688 | 100 | gp50/TROP-2 | Homo sapiens |
| 262 | AAH02532 | 795 | 100 | BC002532 epididymal secretory | Homo sapiens |
| | | | | protein (19.5 kD) | |
| 262 | X67698 | 795 | 100 | orf | Homo sapiens |
| 262 | X78134 | 795 | 100 | epididymal secretory protein 14.6 | Macaca |
| | | | | | fascicularis |
| 263 | AK075092 | 233 | 100 | unnamed protein product | Homo sapiens |
| 264 | AAH16692 | 1163 | 100 | BC016692 progesterone receptor | Homo sapiens |
| | | | | membrane component 2 | |
| 264 | AJ002030 | 1163 | 100 | progresterone binding protein | Homo sapiens |
| 264 | AY069921 | 525 | 60 | membrane progesterone receptor- | Oncorhynchus |
| | | | | like protein | mykiss |
| 265 | AAH16041 | 1441 | 100 | BC016041 insulin-like growth | Homo sapiens |
| | | | | factor-binding protein 4 | |
| 265 | M62403 | 1441 | 100 | IGF-BP 4 | Homo sapiens |
| 265 | Y12508 | 1441 | 100 | insulin-like growth factor | Homo sapiens |
| | | | | binding protein 4 | |
| 266 | AK000575 | 734 | 100 | unnamed protein product | Homo sapiens |
| 266 | AK013438 | 484 | 72 | unnamed protein product | Mus musculus |
| 266 | AY116892 | 734 | 100 | HCF-1 beta-propeller interacting | Homo sapiens |
| | | | | protein | |
| 267 | AAH14410 | 2273 | 100 | BC014410 Similar to EGF- | Homo sapiens |
| | | | | containing fibulin-like | |
| | | | | extracellular matrix protein 1 | |
| 267 | AK077302 | 2156 | 93 | unnamed protein product | Mus musculus |
| 267 | AY004330 | 2273 | 100 | EGF-containing fibulin-like | Homo sapiens |
| | | | | extracellular matrix protein 1 | |
| 268 | AF102552 | 11066 | 84 | 270 kDa ankyrin G isoform | Rattus |
| | | | | | norvegicus |
| 268 | L40632 | 7184 | 95 | ankyrin 3 | Mus musculus |
| 268 | U13616 | 20107 | 99 | ankyrin G | Homo sapiens |
| 269 | AC002073 | 3406 | 100 | Lim Kinase | Homo sapiens |
| 269 | AL117466 | 3183 | 100 | hypothetical protein | Homo sapiens |
| 269 | D45906 | 3406 | 100 | LIMK-2 | Homo sapiens |
| 270 | AAH00836 | 633 | 100 | BC000836 CGI-127 protein | Homo sapiens |
| 270 | AF135161_1 | 633 | 100 | AF135161 unknown | Homo sapiens |
| 270 | AF151885_1 | 633 | 100 | AF151885 CGI-127 protein | Homo sapiens |
| 271 | A16768 | 356 | 79 | kunitz type protease inhibitor | synthetic |
| | | | | | construct |
| 271 | BC033174 | 501 | 98 | Similar to collagen, type VI, | Homo sapiens |
| | | | | alpha 3 | |
| 271 | X52022 | 501 | 98 | collagen type VI, alpha 3 chain | Homo sapiens |
| 272 | AAH04440 | 405 | 100 | BC004440 Unknown (protein for | Homo sapiens |
| | | | | MGC: 4033) | |
| 272 | AAH19336 | 424 | 98 | BC019336 Unknown (protein for | Homo sapiens |
| | | | | IMAGE: 3617778) | |
| 272 | U91512 | 399 | 98 | ninjurin | Homo sapiens |
| 273 | AAH18127 | 1250 | 93 | BC018127 Similar to | Homo sapiens |
| | | | | Peptidylglycine alpha-amidating | |
| | | | | monooxygenase | |
| 273 | AB095007 | 1250 | 93 | peptidylglycine alpha-amidating | Homo sapiens |
| | | | | monooxygenase | |
| 273 | AF035320 | 1279 | 100 | peptidylglycine alpha-amidating | Homo sapiens |
| | | | | monooxygenase | |
| 274 | AAH13398 | 6167 | 100 | BC013398 pumilio (Drosophila) | Homo sapiens |
| | | | | homolog 1 | |
| 274 | AF315592_1 | 6167 | 100 | AF315592 Pumilio 1 | Homo sapiens |
| 274 | D43951 | 6233 | 100 | KIAA0099 protein | Homo sapiens |
| 275 | AAH00465 | 826 | 100 | BC000465 growth arrest and DNA- | Homo sapiens |
| | | | | damage-inducible, gamma | |
| 275 | AAH19325 | 826 | 100 | BC019325 growth arrest and DNA- | Homo sapiens |
| | | | | damage-inducible, gamma | |
| 275 | AF494037_1 | 826 | 100 | AF494037 growth arrest and DNA- | Homo sapiens |
| | | | | damage-inducible, gamma | |
| 276 | AAH01955 | 883 | 100 | BC001955 ribosomal protein S10 | Homo sapiens |
| 276 | AAH04334 | 883 | 100 | BC004334 ribosomal protein S10 | Homo sapiens |
| 276 | AAH05012 | 883 | 100 | BC005012 ribosomal protein S10 | Homo sapiens |
| 277 | AAH12740 | 4687 | 99 | BC012740 Similar to dystroglycan | Homo sapiens |
| | | | | 1 (dystrophin-associated | |
| | | | | glycoprotein 1) | |
| 277 | AAH14616 | 4687 | 99 | BC014616 Unknown (protein for | Homo sapiens |
| | | | | MGC: 3618) | |
| 277 | L19711 | 4703 | 100 | dystroglycan | Homo sapiens |
| 278 | AAH20983 | 583 | 100 | BC020983 pituitary tumor- | Homo sapiens |
| | | | | transforming 1 interacting | |
| | | | | protein | |
| 278 | BC031097 | 583 | 100 | pituitary tumor-transforming 1 | Homo sapiens |
| | | | | interacting protein | |
| 278 | BC034250 | 583 | 100 | pituitary tumor-transforming 1 | Homo sapiens |
| | | | | interacting protein | |
| 279 | AAH07082 | 185 | 100 | BC007082 SWI/SNF related, matrix | Homo sapiens |
| | | | | associated, actin dependent | |
| | | | | regulator of chromatin, subfamily | |
| | | | | e, member 1 | |
| 279 | AAH11017 | 185 | 100 | BC011017 SWI/SNF related, matrix | Homo sapiens |
| | | | | associated, actin dependent | |
| | | | | regulator of chromatin, subfamily | |
| | | | | e, member 1 | |
| 279 | AF035262 | 185 | 100 | BAF57 | Homo sapiens |
| 281 | AF099154 | 13656 | 86 | von Willebrand factor | Canis |
| | | | | | familiaris |
| 281 | M25865 | 15510 | 99 | von Willebrand factor | Homo sapiens |
| 281 | X04385 | 15515 | 100 | VWF pre-pro-polypeptide (−22 to | Homo sapiens |
| | | | | 2791) | |
| 282 | AAH17692 | 1500 | 78 | BC017692 Similar to quiescin Q6 | Homo sapiens |
| 282 | AF361868 | 1492 | 78 | sulfhydryl oxidase | Homo sapiens |
| 282 | U97276 | 2262 | 84 | quiescin | Homo sapiens |
| 284 | AAH12123 | 1528 | 100 | BC012123 golgi phosphoprotein 3 | Homo sapiens |
| 284 | AJ296152 | 1528 | 100 | Golgi protein | Homo sapiens |
| 284 | BC033725 | 1528 | 100 | golgi phosphoprotein 3 (coat- | Homo sapiens |
| | | | | protein) | |
| 285 | AAH08726 | 2687 | 100 | BC008726 oxidative-stress | Homo sapiens |
| | | | | responsive 1 | |
| 285 | AB017642 | 2687 | 100 | oxidative-stress responsive 1 | Homo sapiens |
| 285 | AB029024 | 2355 | 100 | KIAA1101 protein | Homo sapiens |
| 286 | AAH00478 | 3403 | 99 | BC000478 heat shock 70 kD protein | Homo sapiens |
| | | | | 9B (mortalin-2) | |
| 286 | BC024034 | 3403 | 99 | heat shock 70 kD protein 9B | Homo sapiens |
| | | | | (mortalin-2) | |
| 286 | BC030634 | 3399 | 99 | heat shock 70 kD protein 9B | Homo sapiens |
| | | | | (mortalin-2) | |
| 287 | BC006834 | 892 | 100 | B-cell translocation gene 1, | Mus musculus |
| | | | | anti-proliferative | |
| 287 | BC018309 | 892 | 100 | B-cell translocation gene 1, | Mus musculus |
| | | | | anti-proliferative | |
| 287 | Z16410 | 892 | 100 | btg1 | Mus musculus |
| 288 | AE003667 | 256 | 81 | CG9318-PA | Drosophila |
| | | | | | melanogaster |
| 288 | AF026213 | 244 | 71 | Hypothetical protein F08F1.7 | Caenorhabditis |
| | | | | | elegans |
| 288 | AY069665 | 256 | 81 | LD44273p | Drosophila |
| | | | | | melanogaster |
| 289 | A03736 | 3879 | 100 | factor XIIIa | Homo sapiens |
| 289 | M14354 | 3879 | 100 | clotting factor XIIIa precursor | Homo sapiens |
| | | | | (EC 2.3.2.13) | |
| 289 | M14539 | 4016 | 99 | factor XIII precursor | Homo sapiens |
| 290 | AAH06541 | 969 | 99 | BC006541 integrin, beta 5 | Homo sapiens |
| 290 | J05633 | 962 | 98 | integrin beta-5 subunit precursor | Homo sapiens |
| 290 | X53002 | 969 | 99 | precusor polypeptide (AA −23 to | Homo sapiens |
| | | | | 776) | |
| 291 | D50929 | 7262 | 100 | The KIAA0139 gene product is | Homo sapiens |
| | | | | related to mouse centrosomin B. | |
| 291 | U58046 | 7262 | 100 | p167 | Homo sapiens |
| 291 | U78311 | 7262 | 100 | translation initiation factor 3 | Homo sapiens |
| | | | | large subunit | |
| 292 | AAH01571 | 2935 | 100 | BC001571 eukaryotic translation | Homo sapiens |
| | | | | initiation factor 3, subunit 8 | |
| | | | | (110 kD) | |
| 292 | AC002544 | 2935 | 100 | Translation initiation factor | Homo sapiens |
| | | | | eIF-p110 | |
| 292 | U46025 | 2935 | 100 | translation intiation factor eIF- | Homo sapiens |
| | | | | 3 p110 subunit | |
| 293 | AB019987 | 6513 | 99 | chromosome-associated | Homo sapiens |
| | | | | polypeptide-C | |
| 293 | AF092564 | 6041 | 99 | chromosome-associated protein-C | Homo sapiens |
| 293 | AL136877 | 6517 | 100 | hypothetical protein | Homo sapiens |
| 294 | AAH01756 | 2880 | 99 | BC001756 phosphoglucomutase 1 | Homo sapiens |
| 294 | AAH19920 | 2873 | 99 | BC019920 Unknown (protein for | Homo sapiens |
| | | | | MGC: 29909) | |
| 294 | M83088 | 2880 | 99 | PGM1 | Homo sapiens |
| 295 | AB020664 | 3759 | 100 | KIAA0857 protein | Homo sapiens |
| 295 | AF334812_1 | 3333 | 100 | AF334812 Rab11 interacting | Homo sapiens |
| | | | | protein Rip11a | |
| 295 | BC035013 | 3303 | 99 | Similar to Rab coupling protein | Homo sapiens |
| 296 | AF356518_1 | 1637 | 100 | AF356518 junctional adhesion | Homo sapiens |
| | | | | molecule 3 precursor | |
| 296 | AF448478_1 | 1637 | 100 | AF448478 junctional adhesional | Homo sapiens |
| | | | | molecule-3 | |
| 296 | AJ416101 | 1870 | 100 | junction adhesion molecule 3 | Homo sapiens |
| 297 | AF130117_48 | 239 | 76 | AF130089 PRO2550 | Homo sapiens |
| 297 | AK000385 | 240 | 76 | unnamed protein product | Homo sapiens |
| 297 | AK090988 | 218 | 71 | unnamed protein product | Homo sapiens |
| 298 | AAH01562 | 3284 | 100 | BC001562 nuclear receptor | Homo sapiens |
| | | | | coactivator 4 | |
| 298 | AL162047 | 3284 | 100 | hypothetical protein | Homo sapiens |
| 298 | L49399 | 3284 | 100 | ORF | Homo sapiens |
| 299 | AAH02356 | 2379 | 100 | BC002356 nucleobindin 1 | Homo sapiens |
| 299 | M96824 | 2326 | 98 | nucleobindin | Homo sapiens |
| 299 | U31342 | 2357 | 99 | nucleobindin | Homo sapiens |
| 300 | AAH17180 | 4694 | 100 | BC017180 Unknown (protein for | Homo sapiens |
| | | | | MGC: 790) | |
| 300 | AK088028 | 4597 | 97 | unnamed protein product | Mus musculus |
| 300 | BC007126 | 4597 | 97 | Unknown (protein for MGC: 5677) | Mus musculus |
| 301 | AAH08099 | 378 | 100 | BC008099 Unknown (protein for | Homo sapiens |
| | | | | IMAGE: 4183312) | |
| 301 | AAH18427 | 214 | 100 | BC018427 Unknown (protein for | Homo sapiens |
| | | | | IMAGE: 4515233) | |
| 302 | AF137053_1 | 2310 | 99 | AF137053 mutant desmin | Homo sapiens |
| 302 | BC032116 | 2313 | 99 | desmin | Homo sapiens |
| 302 | U59167 | 2325 | 100 | desmin | Homo sapiens |
| 303 | AL445795 | 23376 | 99 | heparan sulfate proteoglycan | Homo sapiens |
| | | | | perlecan | |
| 303 | M85289 | 23477 | 99 | heparan sulfate proteoglycan | Homo sapiens |
| 303 | X62515 | 23627 | 99 | Human basement membrane heparan | Homo sapiens |
| | | | | sulfate proteoglycan core protein | |
| 304 | AK025116 | 206 | 48 | unnamed protein product | Homo sapiens |
| 304 | AK057830 | 227 | 49 | unnamed protein product | Homo sapiens |
| 304 | AK090511 | 194 | 51 | unnamed protein product | Homo sapiens |
| 305 | AAH00429 | 309 | 100 | BC000429 chromosome 14 open | Homo sapiens |
| | | | | reading frame 2 | |
| 305 | AAH01944 | 309 | 100 | BC001944 chromosome 14 open | Homo sapiens |
| | | | | reading frame 2 | |
| 305 | AF054175 | 309 | 100 | mitochondrial proteolipid 68MP | Homo sapiens |
| | | | | homolog | |
| 306 | AAH01263 | 2152 | 100 | BC001263 serum/glucocorticoid | Homo sapiens |
| | | | | regulated kinase | |
| 306 | AF153609_1 | 2152 | 100 | AF153609 serine/threonine protein | Homo sapiens |
| | | | | kinase sgk | |
| 306 | AX553549 | 2152 | 100 | unnamed protein product | Homo sapiens |
| 307 | AAH07103 | 710 | 100 | BC007103 small acidic protein | Homo sapiens |
| 307 | AAH16352 | 710 | 100 | BC016352 small acidic protein | Homo sapiens |
| 307 | AAH20937 | 710 | 100 | BC020937 small acidic protein | Homo sapiens |
| 309 | AY043487 | 412 | 100 | selenoprotein SelM | Homo sapiens |
| 309 | AY043488 | 367 | 85 | selenoprotein SelM | Mus musculus |
| 309 | BC019742 | 345 | 85 | Unknown (protein for MGC: 30803) | Mus musculus |
| 310 | AF124719_1 | 1018 | 100 | AF124719 GM2 activator protein | Homo sapiens |
| 310 | X61095 | 1039 | 97 | GM2-activator protein | Homo sapiens |
| 310 | X62078 | 1018 | 100 | GM2 activator protein | Homo sapiens |
| 311 | AB028974 | 1802 | 100 | KIAA1051 protein | Homo sapiens |
| 311 | AX082622 | 1995 | 100 | unnamed protein product | Homo sapiens |
| 311 | AX082622 | 2129 | 100 | unnamed protein product | Homo sapiens |
| 312 | BC026690 | 3964 | 100 | CD97 antigen | Homo sapiens |
| 312 | U76764 | 3796 | 87 | CD97 | Homo sapiens |
| 312 | X84700 | 3951 | 99 | leucocyte antigen CD97 | Homo sapiens |
| 313 | K03195 | 2422 | 99 | glucose transporter glycoprotein | Homo sapiens |
| 313 | M13979 | 2367 | 97 | glucose-transporter protein | Rattus |
| | | | | | norvegicus |
| 313 | M22063 | 2367 | 97 | glucose transporter protein | Rattus |
| | | | | | norvegicus |
| 314 | AAH19297 | 1801 | 100 | BC019297 Unknown (protein for | Homo sapiens |
| | | | | MGC: 4111) | |
| 314 | AK021655 | 1801 | 100 | unnamed protein product | Homo sapiens |
| 314 | AK022757 | 1801 | 100 | unnamed protein product | Homo sapiens |
| 315 | AC005943 | 1522 | 100 | methyl-CpG binding protein MBD3 | Homo sapiens |
| 315 | AF072247 | 1522 | 100 | methyl-CpG binding domain- | Homo sapiens |
| | | | | containing protein MBD3 | |
| 315 | AF072248 | 1427 | 95 | methyl-CpG binding domain- | Mus musculus |
| | | | | containing protein MBD3 | |
| 316 | AB018009 | 2602 | 100 | L-type amino acid transporter 1 | Homo sapiens |
| 316 | AB018542 | 2602 | 100 | CD98 light chain | Homo sapiens |
| 316 | AF104032 | 2602 | 100 | L-type amino acid transporter | Homo sapiens |
| | | | | subunit LAT1 | |
| 318 | AAH10737 | 1911 | 100 | BC010737 Similar to reticulon 4 | Homo sapiens |
| 318 | AAH12619 | 1911 | 100 | BC012619 Unknown (protein for | Homo sapiens |
| | | | | MGC: 13655) | |
| 318 | BC026788 | 1911 | 100 | reticulon 4 | Homo sapiens |
| 319 | AAH02412 | 3766 | 99 | BC002412 inner membrane protein, | Homo sapiens |
| | | | | mitochondrial (mitofilin) | |
| 319 | D21094 | 3766 | 99 | motor protein | Homo sapiens |
| 319 | L42572 | 3771 | 100 | transmembrane protein | Homo sapiens |
| 320 | AAH01925 | 573 | 100 | BC001925 FK506-binding protein 1A | Homo sapiens |
| | | | | (12 kD) | |
| 320 | AAH05147 | 573 | 100 | BC005147 FK506-binding protein 1A | Homo sapiens |
| | | | | (12 kD) | |
| 320 | M93060 | 573 | 100 | FK506-binding protein 12 | Homo sapiens |
| 321 | AF116721_20 | 391 | 100 | AF116620 PRO1068 | Homo sapiens |
| 322 | AK075023 | 1335 | 99 | unnamed protein product | Homo sapiens |
| 322 | AL117442 | 1344 | 100 | hypothetical protein | Homo sapiens |
| 322 | BC002138 | 1302 | 96 | Unknown (protein for | Mus musculus |
| | | | | IMAGE: 3484538) | |
| 323 | AAH07674 | 382 | 100 | BC007674 CD24 antigen (small cell | Homo sapiens |
| | | | | lung carcinoma cluster 4 antigen) | |
| 323 | L33930 | 382 | 100 | signal transducer CD24 | Homo sapiens |
| 323 | M58664 | 382 | 100 | signal transducer CD24 | Homo sapiens |
| 324 | AAH12181 | 4260 | 100 | BC012181 Similar to paired basic | Homo sapiens |
| | | | | amino acid cleaving enzyme | |
| | | | | (furin, membrane associated | |
| | | | | receptor protein) | |
| 324 | U20436 | 4060 | 95 | furin endoprotease | Cricetulus |
| | | | | | griseus |
| 324 | X17094 | 4260 | 100 | furin (AA 1-794) | Homo sapiens |
| 325 | AAH15236 | 876 | 100 | BC015236 hypothetical protein | Homo sapiens |
| 325 | AF335324_1 | 876 | 100 | AF335324 RTP801 | Homo sapiens |
| 325 | AY090097 | 876 | 100 | REDD-1 | Homo sapiens |
| 326 | AK000496 | 332 | 66 | unnamed protein product | Homo sapiens |
| 326 | AK025116 | 327 | 69 | unnamed protein product | Homo sapiens |
| 326 | AK057830 | 358 | 69 | unnamed protein product | Homo sapiens |
| 327 | AAH10058 | 3813 | 100 | BC010058 exostoses (multiple) 2 | Homo sapiens |
| 327 | U62740 | 3813 | 100 | EXT2 | Homo sapiens |
| 327 | U67368 | 3813 | 100 | multiple exostosis 2 | Homo sapiens |
| 328 | AAH08301 | 1967 | 100 | BC008301 tuftelin 1 | Homo sapiens |
| 328 | AF254260_1 | 1967 | 100 | AF254260 tuftelin 1 | Homo sapiens |
| 328 | AF254860 | 1967 | 100 | tuftelin 1 | Homo sapiens |
| 330 | AF272662_1 | 6961 | 80 | AF272662 alpha 1 type V collagen | Rattus |
| | | | | | norvegicus |
| 330 | D90279 | 7452 | 86 | collagen alpha 1(V) chain | Homo sapiens |
| | | | | precursor | |
| 330 | M76729 | 7422 | 85 | pro-alpha-1 type V collagen | Homo sapiens |
| 331 | AAH03155 | 1193 | 76 | BC003155 coatomer protein | Homo sapiens |
| | | | | complex, subunit epsilon | |
| 331 | AAH07250 | 1193 | 76 | BC007250 coatomer protein | Homo sapiens |
| | | | | complex, subunit epsilon | |
| 331 | AAH17285 | 1193 | 76 | BC017285 Unknown (protein for | Homo sapiens |
| | | | | MGC: 29652) | |
| 332 | AX354381 | 347 | 100 | unnamed protein product | Homo sapiens |
| 333 | AF202636_1 | 2164 | 100 | AF202636 angiopoietin-like | Homo sapiens |
| | | | | protein PP1158 | |
| 333 | AX079971 | 2164 | 100 | unnamed protein product | Homo sapiens |
| 333 | BC023647 | 2164 | 100 | angiopoietin-like 4 | Homo sapiens |
| 334 | AAH12777 | 162 | 100 | BC012777 Unknown (protein for | Homo sapiens |
| | | | | MGC: 16395) | |
| 334 | AK056767 | 1189 | 100 | unnamed protein product | Homo sapiens |
| 334 | AL831913 | 713 | 85 | hypothetical protein | Homo sapiens |
| 335 | AAH01270 | 1896 | 100 | BC001270 SH3-domain GRB2-like 1 | Homo sapiens |
| 335 | U65999 | 1896 | 100 | SH3-containing protein EEN | Homo sapiens |
| 335 | X99656 | 1896 | 100 | SH3-containing Grb-2-like 1 | Homo sapiens |
| 336 | AAH00385 | 4505 | 100 | BC000385 Unknown (protein for | Homo sapiens |
| | | | | MGC: 8429) | |
| 336 | AF102803 | 4505 | 100 | alphaE-catenin | Homo sapiens |
| 336 | D13866 | 4505 | 100 | alpha-catenin | Homo sapiens |
| 337 | AAH07564 | 601 | 90 | BC007564 annexin All | Homo sapiens |
| 337 | AJ278464 | 601 | 90 | annexin All | Homo sapiens |
| 337 | AJ278465 | 601 | 90 | annexin All | Homo sapiens |
| 338 | AAH01491 | 1470 | 99 | BC001491 heme oxygenase | Homo sapiens |
| | | | | (decycling) 1 | |
| 338 | X06985 | 1475 | 100 | heme oxygenase (AA 1-288) | Homo sapiens |
| 338 | Z82244 | 1475 | 100 | bK286B10.2 (Heme Oxygenase | Homo sapiens |
| | | | | (decycling) 1 (HO-1 EC | |
| | | | | 1.14.99.3)) | |
| 340 | AAH13910 | 1671 | 100 | BC013910 Similar to death | Homo sapiens |
| | | | | effector domain-containing | |
| 340 | AAH16724 | 1671 | 100 | BC016724 death effector domain- | Homo sapiens |
| | | | | containing | |
| 340 | AJ010973 | 1671 | 100 | DEDD protein | Homo sapiens |
| 341 | A16794 | 881 | 95 | cDNA isolated for this protein | Homo sapiens |
| | | | | using a monoclonal antibody | |
| | | | | directed against the p27k | |
| | | | | prosomal protein | |
| 341 | BC022354 | 956 | 97 | proteasome (prosome, macropain) | Homo sapiens |
| | | | | subunit, alpha type 6 | |
| 341 | D10755 | 956 | 97 | proteasome subunit R-IOTA | Rattus sp. |
| 342 | AAH09581 | 2041 | 100 | BC009581 hydroxysteroid (17-beta) | Homo sapiens |
| | | | | dehydrogenase 2 | |
| 342 | L11708 | 2041 | 100 | 17 beta hydroxysteroid | Homo sapiens |
| | | | | dehydrogenase type 2 | |
| 342 | L40802 | 2041 | 100 | 17-hydroxysteroid dehydrogenase | Homo sapiens |
| 343 | AAH08452 | 586 | 100 | BC008452 ATPase, H+ transporting, | Homo sapiens |
| | | | | lysosomal (vacuolar proton pump), | |
| | | | | member J | |
| 343 | AF038954 | 586 | 100 | vacuolar H(+)-ATPase subunit | Homo sapiens |
| 343 | S82464 | 573 | 96 | vacuolar H(+)-ATPase subunit; V- | Bos taurus |
| | | | | ATPase subunit; M16 | |
| 344 | AE003452 | 193 | 35 | CG9350-PB | Drosophila |
| | | | | | melanogaster |
| 344 | AJ510148 | 571 | 72 | mitochondrial NADH: ubiquinone | Bos taurus |
| | | | | oxidoreductase B14.7 subunit | |
| 344 | AK008201 | 532 | 65 | unnamed protein product | Mus musculus |
| 345 | AB020685 | 3216 | 100 | KIAA0878 protein | Homo sapiens |
| 345 | AK006650 | 2065 | 93 | unnamed protein product | Mus musculus |
| 345 | BC041337 | 3212 | 99 | Rho-related BTB domain containing 3 | Homo sapiens |
| 346 | AB058749 | 1868 | 100 | KIAA1846 protein | Homo sapiens |
| 346 | AK092295 | 1868 | 100 | unnamed protein product | Homo sapiens |
| 346 | AL035661 | 1868 | 100 | dJ568C11.3 (novel AMP-binding | Homo sapiens |
| | | | | enzyme similar to acetyl-coenzyme | |
| | | | | A synthethase (acetate-coA | |
| | | | | ligase)) | |
| 347 | AF009242 | 1168 | 100 | proline-rich Gla protein 1 | Homo sapiens |
| 347 | AF419154 | 577 | 52 | mitotic phosphoprotein 77 | Xenopus |
| | | | | | laevis |
| 347 | BC030786 | 1169 | 84 | proline-rich Gla (G- | Homo sapiens |
| | | | | carboxyglutamic acid) polypeptide 1 | |
| 348 | AF000652 | 1503 | 99 | syntenin | Homo sapiens |
| 348 | AF006636 | 1508 | 100 | melanoma differentiation | Homo sapiens |
| | | | | associated protein-9 | |
| 348 | U83463 | 1503 | 99 | scaffold protein Pbp1 | Homo sapiens |
| 349 | AF312032 | 5200 | 99 | ephrin type-B receptor 4 | Homo sapiens |
| | | | | precursor | |
| 349 | AY056047 | 5200 | 99 | receptor protein tyrosine kinase | Homo sapiens |
| | | | | EphB4 | |
| 349 | U07695 | 5196 | 99 | tyrosine kinase | Homo sapiens |
| 350 | AF015553 | 4955 | 99 | TFII-I protein | Homo sapiens |
| 350 | AF038969 | 4949 | 99 | general transcription factor 2-I | Homo sapiens |
| 350 | Y14946 | 4949 | 99 | SPIN protein | Homo sapiens |
| 351 | AAH07452 | 1442 | 100 | BC007452 Similar to WW domain | Homo sapiens |
| | | | | binding protein 2 | |
| 351 | AAH10616 | 1442 | 100 | BC010616 Unknown (protein for | Homo sapiens |
| | | | | MGC: 18269) | |
| 351 | U79458 | 1458 | 100 | WW domain binding protein-2 | Homo sapiens |
| 352 | AB097511 | 3996 | 93 | hypothetical protein | Macaca |
| | | | | | fascicularis |
| 352 | AL359292 | 7373 | 99 | dJ448K1.1.1 (absent in melanoma | Homo sapiens |
| | | | | 1, isoform 1) | |
| 352 | U83115 | 8577 | 100 | non-lens beta gamma-crystallin | Homo sapiens |
| | | | | like protein | |
| 353 | AAH05921 | 357 | 86 | BC005921 chorionic | Homo sapiens |
| | | | | somatomammotropin hormone 1 | |
| | | | | (placental lactogen) | |
| 353 | AAH20756 | 357 | 86 | BC020756 chorionic | Homo sapiens |
| | | | | somatomammotropin hormone 1 | |
| | | | | (placental lactogen) | |
| 353 | AAH22044 | 357 | 86 | BC022044 chorionic | Homo sapiens |
| | | | | somatomammotropin hormone 2 | |
| 354 | AY158924 | 650 | 100 | histone protein Hist2h2aa2 | Mus musculus |
| 354 | AY158925 | 650 | 100 | histone protein Hist2h2aa1 | Mus musculus |
| 354 | AY158953 | 650 | 100 | histone protein Hist2h3c2 | Mus musculus |
| 355 | AF226614_1 | 2929 | 100 | AF226614 ferroportin1 | Homo sapiens |
| 355 | AF231121_1 | 2929 | 100 | AF231121 iron-regulated | Homo sapiens |
| | | | | transporter IREG1 | |
| 355 | BC037733 | 2929 | 100 | solute carrier family 11 (proton- | Homo sapiens |
| | | | | coupled divalent metal ion | |
| | | | | transporters), member 3 | |
| 356 | AAH01693 | 709 | 100 | BC001693 lectin, galactoside- | Homo sapiens |
| | | | | binding, soluble, 1 (galectin 1) | |
| 356 | AAH20675 | 709 | 100 | BC020675 lectin, galactoside- | Homo sapiens |
| | | | | binding, soluble, 1 (galectin 1) | |
| 356 | X14829 | 709 | 100 | beta-galactoside-binding lectin | Homo sapiens |
| | | | | (AA 1-135) | |
| 357 | AF151794 | 521 | 39 | pol protein | Phascolarctos |
| | | | | | cinereus |
| 357 | AJ293657 | 496 | 37 | polymerase | Porcine |
| | | | | | endogenous |
| | | | | | type C |
| | | | | | retrovirus |
| 357 | M26927 | 517 | 39 | pol polyprotein | Gibbon ape |
| | | | | | leukemia |
| | | | | | virus |
| 358 | AAD18076 | 689 | 99 | AF129756 G6c | Homo sapiens |
| 358 | AJ012008 | 689 | 99 | Ly6-C protein | Homo sapiens |
| 358 | AJ315533 | 689 | 99 | LY6G6C protein | Homo sapiens |
| 359 | AAH11682 | 2559 | 100 | BC011682 Similar to cathepsin F | Homo sapiens |
| 359 | AF132894_1 | 2559 | 100 | AF132894 cathepsin F | Homo sapiens |
| 359 | AJ007331 | 2559 | 100 | cysteine proteinase | Homo sapiens |
| 360 | AF151048_1 | 407 | 87 | AF151048 HSPC214 | Homo sapiens |
| 360 | AF247565_1 | 423 | 91 | AF247565 anaphase promoting | Homo sapiens |
| | | | | complex subunit 11 | |
| 360 | AF247789_1 | 423 | 91 | AF247789 putative anaphase- | Homo sapiens |
| | | | | promoting complex subunit APC11 | |
| 361 | AAH11811 | 559 | 92 | BC011811 Unknown (protein for | Homo sapiens |
| | | | | MGC: 20260) | |
| 361 | AF218016_1 | 432 | 76 | AF218016 unknown | Homo sapiens |
| 362 | AAH17201 | 634 | 98 | BC017201 insulin-like growth | Homo sapiens |
| | | | | factor binding protein 7 | |
| 362 | L19182 | 634 | 98 | MAC25 | Homo sapiens |
| 362 | S75725 | 634 | 98 | prostacyclin-stimulating factor; | Homo sapiens |
| | | | | PGI2-stimulating factor; PSF | |
| 364 | AF126110_1 | 643 | 96 | AF126110 fibulin-1 isoform D | Homo sapiens |
| | | | | precursor | |
| 364 | BC022497 | 643 | 96 | fibulin 1 | Homo sapiens |
| 364 | U01244 | 643 | 96 | fibulin-1D | Homo sapiens |
| 365 | AAH05839 | 414 | 97 | BC005839 follistatin-like 3 | Homo sapiens |
| | | | | (secreted glycoprotein) | |
| 365 | BC033119 | 414 | 97 | follistatin-like 3 (secreted | Homo sapiens |
| | | | | glycoprotein) | |
| 365 | U76702 | 414 | 97 | follistatin-related protein FLRG | Homo sapiens |
| 366 | AAH00163 | 601 | 99 | BC000163 vimentin | Homo sapiens |
| 366 | BC030573 | 601 | 99 | Unknown (protein for MGC: 16183) | Homo sapiens |
| 366 | X56134 | 601 | 99 | vimentin | Homo sapiens |
| 367 | AB018265 | 5555 | 100 | KIAA0722 protein | Homo sapiens |
| 367 | AF045458 | 5460 | 100 | serine/threonine kinase ULK1 | Homo sapiens |
| 367 | AF072370_1 | 4865 | 89 | AF072370 UNC51.1 serine/threonine | Mus musculus |
| | | | | kinase | |
| 368 | AAH00865 | 227 | 100 | BC000865 Unknown (protein for | Homo sapiens |
| | | | | IMAGE: 3460093) | |
| 369 | AAH02978 | 279 | 91 | BC002978 CD81 antigen (target of | Homo sapiens |
| | | | | antiproliferative antibody 1) | |
| 369 | AF116600 | 279 | 91 | CD81 | Pan |
| | | | | | troglodytes |
| 369 | M33680 | 279 | 91 | 26-kDa cell surface protein TAPA-1 | Homo sapiens |
| 370 | AAH15156 | 678 | 100 | BC015156 ferritin, heavy | Homo sapiens |
| | | | | polypeptide 1 | |
| 370 | AAH16009 | 678 | 100 | BC016009 ferritin, heavy | Homo sapiens |
| | | | | polypeptide 1 | |
| 370 | AAH16857 | 678 | 100 | BC016857 ferritin, heavy | Homo sapiens |
| | | | | polypeptide 1 | |
| 371 | AAH08012 | 298 | 67 | BC008012 eukaryotic translation | Homo sapiens |
| | | | | elongation factor 1 delta | |
| | | | | (guanine nucleotide exchange | |
| | | | | protein) | |
| 371 | AAH09907 | 298 | 67 | BC009907 eukaryotic translation | Homo sapiens |
| | | | | elongation factor 1 delta | |
| | | | | (guanine nucleotide exchange | |
| | | | | protein) | |
| 371 | AAH12819 | 298 | 67 | BC012819 eukaryotic translation | Homo sapiens |
| | | | | elongation factor 1 delta | |
| | | | | (guanine nucleotide exchange | |
| | | | | protein) | |
| 372 | AAH20756 | 470 | 83 | BC020756 chorionic | Homo sapiens |
| | | | | somatomammotropin hormone 1 | |
| | | | | (placental lactogen) | |
| 372 | AAH22044 | 470 | 83 | BC022044 chorionic | Homo sapiens |
| | | | | somatomammotropin hormone 2 | |
| 372 | M15894 | 461 | 92 | chorionic somatomammotropin | Homo sapiens |
| | | | | precursor | |
| 373 | A65264 | 705 | 100 | unnamed protein product | unidentified |
| 373 | AAH01288 | 574 | 93 | BC001288 Similar to decay | Homo sapiens |
| | | | | accelerating factor for | |
| | | | | complement (CD55, Cromer blood | |
| | | | | group system) | |
| 373 | M31516 | 574 | 93 | decay-accelerating factor | Homo sapiens |
| 374 | AF289553_1 | 193 | 75 | AF289553 unknown | Homo sapiens |
| 374 | AK096998 | 202 | 64 | unnamed protein product | Homo sapiens |
| 374 | AK097342 | 207 | 78 | unnamed protein product | Homo sapiens |
| 375 | AAH05326 | 499 | 98 | BC005326 ribosomal protein L27a | Homo sapiens |
| 375 | AAH20169 | 499 | 98 | BC020169 Unknown (protein for | Homo sapiens |
| | | | | IMAGE: 3543815) | |
| 375 | U14968 | 499 | 98 | ribosomal protein L27a | Homo sapiens |
| 377 | AAH21297 | 282 | 91 | BC021297 Similar to Dynein heavy | Homo sapiens |
| | | | | chain 64C | |
| 377 | AB002323 | 282 | 91 | KIAA0325 | Homo sapiens |
| 377 | AY004877 | 267 | 94 | cytoplasmic dynein heavy chain | Mus musculus |
| 378 | BC026018 | 643 | 100 | Similar to laminin, beta 1 | Homo sapiens |
| 378 | M20206 | 643 | 100 | laminin B1 | Homo sapiens |
| 378 | M55370 | 643 | 100 | laminin B1 | Homo sapiens |
| 379 | AF061658 | 585 | 91 | cytidine deaminase | Homo sapiens |
| 379 | AJ000474 | 585 | 91 | cytidine deaminase | Homo sapiens |
| 379 | L27943 | 585 | 91 | cytidine deaminase | Homo sapiens |
| 380 | AAH18986 | 412 | 73 | BC018986 Unknown (protein for | Homo sapiens |
| | | | | MGC: 20092) | |
| 380 | AF229830 | 412 | 73 | prostaglandin dehydrogenase | Papio |
| | | | | | hamadryas |
| 380 | U63296 | 412 | 73 | 15-hydroxyprostaglandin | Homo sapiens |
| | | | | dehydrogenase | |
| 381 | AAH00749 | 507 | 100 | BC000749 lactate dehydrogenase A | Homo sapiens |
| 381 | AAH01829 | 507 | 100 | BC001829 lactate dehydrogenase A | Homo sapiens |
| 381 | X03077 | 507 | 100 | lactate dehydrogenase-A | Homo sapiens |
| 382 | AAH00903 | 749 | 84 | BC000903 high-mobility group | Homo sapiens |
| | | | | (nonhistone chromosomal) protein 2 | |
| 382 | AAH01063 | 749 | 84 | BC001063 high-mobility group | Homo sapiens |
| | | | | (nonhistone chromosomal) protein 2 | |
| 382 | X62534 | 749 | 84 | HMG-2 | Homo sapiens |
| 383 | AK075214 | 338 | 91 | unnamed protein product | Homo sapiens |
| 383 | AX417526 | 338 | 91 | unnamed protein product | Homo sapiens |
| 383 | AX417528 | 338 | 91 | unnamed protein product | Homo sapiens |
| 384 | AF135060 | 735 | 96 | fibrillin-2 | Rattus |
| | | | | | norvegicus |
| 384 | U03272 | 745 | 99 | fibrillin-2 | Homo sapiens |
| 384 | X62009 | 745 | 98 | fibrillin 5 | Homo sapiens |
| 385 | AAH13083 | 478 | 100 | BC013083 Similar to cystatin C | Homo sapiens |
| | | | | (amyloid angiopathy and cerebral | |
| | | | | hemorrhage) | |
| 385 | X12763 | 478 | 100 | ompA - cystatin C fusion | synthetic |
| | | | | preprotein (AA −21 to 120) | construct |
| 385 | X61681 | 478 | 100 | cystatin C | Homo sapiens |
| 387 | AF135060 | 523 | 89 | fibrillin-2 | Rattus |
| | | | | | norvegicus |
| 387 | L39790 | 532 | 90 | fibrillin 2 | Mus musculus |
| 387 | U03272 | 597 | 100 | fibrillin-2 | Homo sapiens |
| 388 | M65149 | 1200 | 85 | CELF | Rattus |
| | | | | | norvegicus |
| 388 | M83667 | 1430 | 100 | NF-IL6-beta protein | Homo sapiens |
| 388 | S63168 | 1418 | 99 | CCAAT/enhancer-binding protein | Homo sapiens |
| | | | | delta; C/EBP delta | |
| 389 | AF186111_1 | 228 | 52 | AF186111 NOTCH4-like protein | Homo sapiens |
| 389 | AL512735 | 228 | 52 | hypothetical protein | Homo sapiens |
| 389 | AX133831 | 228 | 52 | unnamed protein product | Homo sapiens |
| 390 | M13452 | 641 | 90 | lamin A protein | Homo sapiens |
| 390 | X03444 | 631 | 95 | put. lamin A precursor (aa 1-702) | Homo sapiens |
| 390 | X66870 | 629 | 84 | lamin A | Rattus |
| | | | | | norvegicus |
| 391 | U14631 | 244 | 100 | 11 beta-hydroxysteroid | Homo sapiens |
| | | | | dehydrogenase type II | |
| 391 | U26726 | 244 | 100 | 11-beta-hydroxysteroid | Homo sapiens |
| | | | | dehydrogenase type 2 | |
| 391 | U27317 | 244 | 100 | 11 beta-hydroxysteroid | Homo sapiens |
| | | | | dehydrogenase 2 | |
| 392 | AB049946 | 666 | 100 | mitochondrial ribosomal protein | Homo sapiens |
| | | | | S15 | |
| 392 | AF265439_1 | 415 | 100 | AF265439 DC37 | Homo sapiens |
| 392 | BC031336 | 666 | 100 | mitochondrial ribosomal protein | Homo sapiens |
| | | | | S15 | |
| 393 | AAH13733 | 333 | 92 | BC013733 px19-like protein | Homo sapiens |
| 393 | AAH13748 | 333 | 92 | BC013748 px19-like protein | Homo sapiens |
| 393 | AF201925_1 | 333 | 92 | AF201925 PRELI | Homo sapiens |
| 394 | AAH02362 | 694 | 100 | BC002362 lactate dehydrogenase B | Homo sapiens |
| 394 | AAH15122 | 694 | 100 | BC015122 lactate dehydrogenase B | Homo sapiens |
| 394 | Y00711 | 694 | 100 | lactate dehydrogenase B (AA 1-334) | Homo sapiens |
| 395 | AAH03070 | 427 | 76 | BC003070 GATA-binding protein 3 | Homo sapiens |
| 395 | AAH06793 | 427 | 76 | BC006793 GATA-binding protein 3 | Homo sapiens |
| 395 | X55037 | 427 | 76 | GATA-3 | Homo sapiens |
| 396 | AF269289_1 | 287 | 94 | AF269289 unknown | Homo sapiens |
| 397 | AAH07728 | 376 | 80 | BC007728 Unknown (protein for | Homo sapiens |
| | | | | MGC: 12671) | |
| 397 | AL080102 | 376 | 80 | hypothetical protein | Homo sapiens |
| 397 | U49436 | 376 | 80 | translation initiation factor 5 | Homo sapiens |
| 398 | AAH03190 | 472 | 94 | BC003190 p75NTR-associated cell | Homo sapiens |
| | | | | death executor; ovarian granulosa | |
| | | | | cell protein (13 kD) | |
| 398 | AF18704_1 | 472 | 94 | AF187064 p75NTR-associated cell | Homo sapiens |
| | | | | death executor; NADE | |
| 398 | M38188 | 472 | 94 | unknown | Homo sapiens |
| 399 | AB037767 | 5427 | 99 | KIAA1346 protein | Homo sapiens |
| 399 | AF207664_1 | 5274 | 100 | AF207664 matrix metalloprotease | Homo sapiens |
| 399 | AP001697 | 5274 | 100 | metalloprotease with | Homo sapiens |
| | | | | thrombospondin type 1 motifs | |
| 400 | U90938 | 213 | 76 | Fc gamma receptor IIc1 | Homo sapiens |
| 400 | X17652 | 213 | 76 | IgG Fc receptor | Homo sapiens |
| 400 | X17652 | 213 | 76 | IgG Fc receptor | Homo sapiens |
| 401 | AAH01768 | 390 | 93 | BC001768 neuronatin | Homo sapiens |
| 401 | AL109614 | 390 | 93 | bA425M5.3.1 (neuronatin (isoform | Homo sapiens |
| | | | | 1)) | |
| 401 | U31767 | 390 | 93 | neuronatin alpha | Homo sapiens |
| 402 | AF151373_1 | 328 | 82 | AF151373 nucleolin-related | Rattus |
| | | | | protein NRP | norvegicus |
| 402 | M15825 | 281 | 73 | nucleolin, C23 | Cricetulus |
| | | | | | griseus |
| 402 | M55022 | 286 | 75 | nucleolin | Rattus |
| | | | | | norvegicus |
| 403 | AAH13428 | 558 | 86 | BC013428 PP1201 protein | Homo sapiens |
| 403 | AF193045_1 | 558 | 86 | AF193045 unknown | Homo sapiens |
| 403 | BC026348 | 558 | 86 | PP1201 protein | Homo sapiens |
| 404 | AF393832_1 | 721 | 94 | AF393832 beta-actin | Morulius |
| | | | | | calbasu |
| 404 | AY039657 | 721 | 94 | beta-actin | Chrysophrys |
| | | | | | auratus |
| 404 | AY148350 | 721 | 94 | actin | Dicentrarchus |
| | | | | | labrax |
| 405 | AK055593 | 370 | 98 | unnamed protein product | Homo sapiens |
| 406 | AAH21233 | 1036 | 100 | BC021233 ATP synthase, H+ | Homo sapiens |
| | | | | transporting, mitochondrial F1 | |
| | | | | complex, O subunit (oligomycin | |
| | | | | sensitivity conferring protein) | |
| 406 | BC022865 | 1036 | 100 | ATP synthase, H+ transporting, | Homo sapiens |
| | | | | mitochondrial F1 complex, O | |
| | | | | subunit (oligomycin sensitivity | |
| | | | | conferring protein) | |
| 406 | X83218 | 1036 | 100 | ATP synthase, oligomycin | Homo sapiens |
| | | | | sensitivity conferring protein | |
| 407 | AAH04368 | 1221 | 99 | BC004368 proteasome (prosome, | Homo sapiens |
| | | | | macropain) activator subunit 2 | |
| | | | | (PA28 beta) | |
| 407 | AAH19885 | 1221 | 99 | BC019885 proteasome (prosome, | Homo sapiens |
| | | | | macropain) activator subunit 2 | |
| | | | | (PA28 beta) | |
| 407 | D45248 | 1226 | 100 | proteasome activator hPA28 suunit | Homo sapiens |
| | | | | beta | |
| 408 | AF090306 | 2306 | 100 | retinoblastoma binding protein | Rattus |
| | | | | | norvegicus |
| 408 | U35143 | 2306 | 100 | retinoblastoma-binding protein | Homo sapiens |
| | | | | RbAp46 | |
| 408 | X72841 | 2306 | 100 | IEF 7442 | Homo sapiens |
| 409 | AAH00413 | 1748 | 100 | BC000413 eukaryotic translation | Homo sapiens |
| | | | | initiation factor 3, subunit 2 | |
| | | | | (beta, 36 kD) | |
| 409 | AAH03140 | 1748 | 100 | BC003140 eukaryotic translation | Homo sapiens |
| | | | | initiation factor 3, subunit 2 | |
| | | | | (beta, 36 kD) | |
| 409 | U39067 | 1748 | 100 | translation initiation factor | Homo sapiens |
| | | | | eIF3 p36 subunit | |
| 410 | AF042166 | 13721 | 100 | beta-filamin | Homo sapiens |
| 410 | AF043045 | 13709 | 99 | actin-binding protein homolog | Homo sapiens |
| | | | | ABP-278 | |
| 410 | AF191633 | 13721 | 100 | filamin | Homo sapiens |
| 411 | AF165515_1 | 1862 | 100 | AF165515 ancient ubiquitous | Homo sapiens |
| | | | | protein 1 precursor | |
| 411 | AK023983 | 1935 | 100 | unnamed protein product | Homo sapiens |
| 411 | BC033646 | 1862 | 100 | ancient ubiquitous protein 1 | Homo sapiens |
| 412 | AB009865 | 1347 | 100 | Angiopoietin-2 | Homo sapiens |
| 412 | AF187858_1 | 1335 | 99 | AF187858 angiopoietin-2 isoform-1 | Homo sapiens |
| 412 | AF218015_1 | 1347 | 100 | AF218015 unknown | Homo sapiens |
| 413 | AF035718 | 914 | 99 | mesoderm-specific basic-helix- | Homo sapiens |
| | | | | loop-helix protein; Pod-1 | |
| 413 | AL356109 | 917 | 100 | bA373A10.1 (transcription factor | Homo sapiens |
| | | | | 21) | |
| 413 | BC025697 | 917 | 100 | transcription factor 21 | Homo sapiens |
| 414 | M38690 | 1200 | 100 | CD9 antigen | Homo sapiens |
| 414 | S60489 | 1200 | 100 | CD9 antigen | Homo sapiens |
| 414 | X60111 | 1200 | 100 | MRP-1 (motility related protein) | Homo sapiens |
| 415 | AAH02368 | 4629 | 99 | BC002368 proteasome (prosome, | Homo sapiens |
| | | | | macropain) 26S subunit, non- | |
| | | | | ATPase, 2 | |
| 415 | AAH02997 | 4629 | 99 | BC002997 proteasome (prosome, | Homo sapiens |
| | | | | macropain) 26S subunit, non- | |
| | | | | ATPase, 2 | |
| 415 | D78151 | 4637 | 100 | human 26S proteasome subunit p97 | Homo sapiens |
| 416 | AAH00182 | 1622 | 100 | BC000182 annexin A4 | Homo sapiens |
| 416 | AAH11659 | 1622 | 100 | BC011659 Similar to annexin A4 | Homo sapiens |
| 416 | D78152 | 1622 | 100 | annexin IV (carbohydrtate-binding | Homo sapiens |
| | | | | protein p33/41) | |
| 417 | AAH03064 | 4016 | 99 | BC003064 disabled (Drosophila) | Homo sapiens |
| | | | | homolog 2 (mitogen-responsive | |
| | | | | phosphoprotein) | |
| 417 | AF205890 | 4028 | 100 | disabled-2 | Homo sapiens |
| 417 | U39050 | 4028 | 100 | DOC-2 | Homo sapiens |
| 418 | AAH07075 | 780 | 100 | BC007075 hemoglobin, beta | Homo sapiens |
| 418 | U01317 | 780 | 100 | beta-globin | Homo sapiens |
| 418 | V00499 | 780 | 100 | beta globin | Homo sapiens |
| 419 | AAH21557 | 4093 | 99 | BC021557 transmembrane protein 8 | Homo sapiens |
| | | | | (five membrane-spanning domains) | |
| 419 | AB045292 | 4100 | 100 | M83 protein | Homo sapiens |
| 419 | AE006463_7 | 4088 | 99 | AE006463 M83 | Homo sapiens |
| 420 | AF193048_1 | 946 | 100 | AF193048 unknown | Homo sapiens |
| 421 | AF151980_1 | 2009 | 100 | AF151980 connexin 43 | Homo sapiens |
| 421 | BC026329 | 2009 | 100 | gap junction protein, alpha 1, | Homo sapiens |
| | | | | 43 kD (connexin 43) | |
| 421 | X52947 | 2009 | 100 | gap junction protein (AA 1-382) | Homo sapiens |
| 422 | AJ313463 | 1258 | 100 | adipsin/complement factor D | Homo sapiens |
| | | | | precursor | |
| 422 | BC034529 | 1249 | 100 | Unknown (protein for | Homo sapiens |
| | | | | IMAGE: 4780594) | |
| 422 | M84526 | 1198 | 98 | adipsin/complement factor D | Homo sapiens |
| 423 | AF258549_1 | 533 | 100 | AF258549 PP1292 | Homo sapiens |
| 424 | AF217963_1 | 4215 | 99 | AF217963 NRAGE | Homo sapiens |
| 424 | AF258554_1 | 4223 | 100 | AF258554 PP2250 | Homo sapiens |
| 424 | BC032473 | 4223 | 100 | melanoma antigen, family D, 1 | Homo sapiens |
| 425 | AAH14635 | 635 | 100 | BC014635 Similar to SH3-domain, | Homo sapiens |
| | | | | GRB2-like, endophilin B2 | |
| 425 | AF257319_1 | 635 | 100 | AF257319 SH3-containing protein | Homo sapiens |
| | | | | SH3GLB2 | |
| 425 | AF258589_1 | 635 | 100 | AF258589 PP578 | Homo sapiens |
| 426 | AAH03390 | 707 | 100 | BC003390 hypothetical protein | Homo sapiens |
| 426 | AAH14334 | 707 | 100 | BC014334 Unknown (protein for | Homo sapiens |
| | | | | MGC: 22874) | |
| 426 | AF275807_1 | 822 | 100 | AF275807 PNAS-110 | Homo sapiens |
| 427 | AAH05238 | 469 | 100 | BC005238 FXYD domain-containing | Homo sapiens |
| | | | | ion transport regulator 3 | |
| 427 | U28249 | 411 | 75 | 11 kD protein | Homo sapiens |
| 427 | X93036 | 469 | 100 | MAT8 protein | Homo sapiens |
| 428 | AF014402 | 1500 | 100 | type-2 phosphatidic acid | Homo sapiens |
| | | | | phosphatase alpha-1 | |
| 428 | BC039847 | 1500 | 100 | Similar to phosphatidic acid | Homo sapiens |
| | | | | phosphatase type 2A | |
| 428 | Y14436 | 1500 | 100 | phosphatidic acid phosphatase | Homo sapiens |
| | | | | type 2 | |
| 429 | AL163249 | 2729 | 99 | T-complex protein 1 theta subunit | Homo sapiens |
| 429 | D13627 | 2730 | 99 | KIAA0002 | Homo sapiens |
| 429 | D42052 | 2734 | 100 | predicted protein of 548 amino | Homo sapiens |
| | | | | acids | |
| 430 | AAH01312 | 2306 | 100 | BC001312 protein disulfide | Homo sapiens |
| | | | | isomerase-related protein | |
| 430 | BC006865 | 2220 | 95 | Similar to protein disulfide | Mus musculus |
| | | | | isomerase-related protein | |
| 430 | D49489 | 2306 | 100 | human P5 | Homo sapiens |
| 431 | AF439513_1 | 7917 | 91 | AF439513 pregnancy-associated | Mus musculus |
| | | | | plasma protein-A | |
| 431 | U28727 | 8980 | 100 | pregnancy-associated plasma | Homo sapiens |
| | | | | protein-A preproform | |
| 431 | X68280 | 8574 | 100 | unnamed protein product | Homo sapiens |
| 432 | AAH01936 | 1357 | 100 | BC001936 Similar to BCL2- | Homo sapiens |
| | | | | associated athanogene | |
| 432 | AAH14774 | 1357 | 100 | BC014774 Unknown (protein for | Homo sapiens |
| | | | | MGC: 17086) | |
| 432 | AF022224 | 1357 | 100 | Bcl-2-binding protein | Homo sapiens |
| 433 | AAH04490 | 1953 | 100 | BC004490 v-fos FBJ murine | Homo sapiens |
| | | | | osteosarcoma viral oncogene | |
| | | | | homolog | |
| 433 | AF111167 | 1953 | 100 | cfos | Homo sapiens |
| 433 | V01512 | 1953 | 100 | c-fos | Homo sapiens |
| 434 | AAH20235 | 3282 | 99 | BC020235 Unknown (protein for | Homo sapiens |
| | | | | MGC: 31939) | |
| 434 | M19645 | 3314 | 100 | GRP78 precursor | Homo sapiens |
| 434 | X87949 | 3314 | 100 | BiP | Homo sapiens |
| 435 | AAH14433 | 2640 | 100 | BC014433 Unknown (protein for | Homo sapiens |
| | | | | MGC: 2159) | |
| 435 | BC036000 | 2640 | 100 | Unknown (protein for | Homo sapiens |
| | | | | IMAGE: 4712175) | |
| 435 | U42068 | 2640 | 100 | P58 | Homo sapiens |
| 436 | BC032722 | 1478 | 100 | tumor necrosis factor (ligand) | Homo sapiens |
| | | | | superfamily, member 10 | |
| 436 | U37518 | 1478 | 100 | TNF-related apoptosis inducing | Homo sapiens |
| | | | | ligand TRAIL | |
| 436 | U57059 | 1478 | 100 | Apo-2 ligand | Homo sapiens |
| 437 | AAH01022 | 1591 | 100 | BC001022 pyrophosphatase | Homo sapiens |
| | | | | (inorganic) | |
| 437 | AF119665_1 | 1591 | 100 | AF119665 inorganic | Homo sapiens |
| | | | | pyrophosphatase | |
| 437 | AF217186_1 | 1591 | 100 | AF217186 inorganic | Homo sapiens |
| | | | | pyrophosphatase 1 | |
| 438 | AAH08743 | 3145 | 100 | BC008743 zyxin | Homo sapiens |
| 438 | AAH09360 | 3145 | 100 | BC009360 zyxin | Homo sapiens |
| 438 | AAH10031 | 3145 | 100 | BC010031 zyxin | Homo sapiens |
| 439 | AAH05901 | 919 | 100 | BC005901 Microfibril-associated | Homo sapiens |
| | | | | glycoprotein-2 | |
| 439 | AF084927 | 919 | 100 | microfibril-associated | Homo sapiens |
| | | | | glycoprotein 2 | |
| 439 | U37283 | 919 | 100 | microfibril-associated | Homo sapiens |
| | | | | glycoprotein-2 MAGP-2 | |
| 440 | AAH00933 | 2015 | 100 | BC000933 isocitrate dehydrogenase | Homo sapiens |
| | | | | 3 (NAD+) gamma | |
| 440 | Z68129 | 2015 | 100 | NAD(H)-specific isocitrate | Homo sapiens |
| | | | | dehydrogenase gamma-subunit | |
| | | | | precursor | |
| 440 | Z68907 | 2015 | 100 | NAD (H)-specific isocitrate | Homo sapiens |
| | | | | dehydrogenase gamma subunit | |
| | | | | precursor | |
| 441 | AAH12265 | 846 | 100 | BC012265 Similar to cofilin 1, | Homo sapiens |
| | | | | non-muscle | |
| 441 | AAH12318 | 846 | 100 | BC012318 Similar to cofilin 1, | Homo sapiens |
| | | | | non-muscle | |
| 441 | AAH18256 | 846 | 100 | BC018256 Similar to cofilin 1, | Homo sapiens |
| | | | | non-muscle | |
| 442 | AB069964 | 861 | 100 | ubiquitin-conjugating enzyme 9 | Gallus |
| | | | | | gallus |
| 442 | AF461016_1 | 861 | 100 | AF461016 ubiquitin-conjugating | Gallus |
| | | | | enzyme | gallus |
| 442 | U88561 | 861 | 100 | E2 ubiquitin conjugating enzyme | Xenopus |
| | | | | | laevis |
| 443 | AAH06249 | 989 | 100 | BC006249 guanylate kinase 1 | Homo sapiens |
| 443 | AAH09914 | 989 | 100 | BC009914 guanylate kinase 1 | Homo sapiens |
| 443 | U66895 | 989 | 100 | guanylate kinase | Homo sapiens |
| 445 | AAH01120 | 1369 | 99 | BC001120 lectin, galactoside- | Homo sapiens |
| | | | | binding, soluble, 3 (galectin 3) | |
| 445 | AF031425 | 1369 | 99 | galectin 3 | Homo sapiens |
| 445 | M35368 | 1377 | 100 | galactose-specific lectin | Homo sapiens |
| 446 | AAH00877 | 615 | 88 | BC000877 hypothetical protein | Homo sapiens |
| | | | | PP5395 | |
| 446 | AF218019_1 | 615 | 88 | AF218019 unknown | Homo sapiens |
| 446 | AF241786_1 | 1125 | 100 | AF241786 NPD013 | Homo sapiens |
| 447 | AAH02503 | 547 | 70 | BC002503 spermidine/spermine N1- | Homo sapiens |
| | | | | acetyltransferase | |
| 447 | AAH08424 | 547 | 70 | BC008424 spermidine/spermine N1- | Homo sapiens |
| | | | | acetyltransferase | |
| 447 | AF251292_1 | 1059 | 100 | AF251292 DC21 | Homo sapiens |
| 448 | AF135157_1 | 745 | 69 | AF135157 complement C1q A chain | Homo sapiens |
| | | | | precursor | |
| 448 | AF260332_1 | 1550 | 100 | AF260332 DC33 | Homo sapiens |
| 448 | BC030153 | 745 | 69 | complement component 1, q | Homo sapiens |
| | | | | subcomponent, alpha polypeptide | |
| 449 | AAH00589 | 770 | 100 | BC000589 CGI-39 protein; cell | Homo sapiens |
| | | | | death-regulatory protein GRIM19 | |
| 449 | AAH09189 | 770 | 100 | BC009189 CGI-39 protein; cell | Homo sapiens |
| | | | | death-regulatory protein GRIM19 | |
| 449 | AF261134_1 | 1196 | 100 | AF261134 CDA016 | Homo sapiens |
| 450 | AAH12296 | 1135 | 99 | BC012296 hypothetical protein | Homo sapiens |
| | | | | FLJ21174 | |
| 450 | AF271783_1 | 1141 | 100 | AF271783 NPD017 | Homo sapiens |
| 450 | AF314542_1 | 1141 | 100 | AF314542 B lymphocyte activation- | Homo sapiens |
| | | | | related protein | |
| 451 | AK090427 | 13940 | 99 | FLJ00343 protein | Homo sapiens |
| 451 | L44140 | 14011 | 100 | filamin | Homo sapiens |
| 451 | X53416 | 14004 | 99 | actin-binding protein | Homo sapiens |
| 452 | AAH12341 | 1492 | 100 | BC012341 Similar to M5-14 protein | Homo sapiens |
| 452 | AL136622 | 1492 | 100 | hypothetical protein | Homo sapiens |
| 452 | AL390090 | 1492 | 100 | c3orf1 hypothetical protein, M5-14 | Homo sapiens |
| | | | | similar to (AE003703)140up | |
| | | | | gene product Drosophila | |
| | | | | melanogaster | |
| 453 | AC005624 | 824 | 100 | MY18_HUMAN | Homo sapiens |
| 453 | AF078077 | 824 | 100 | growth arrest and DNA-damage- | Homo sapiens |
| | | | | inducible protein GADD45beta | |
| 453 | AF087853_1 | 824 | 100 | AF087853 growth arrest and DNA | Homo sapiens |
| | | | | damage inducible protein beta | |
| 454 | AF020185 | 478 | 100 | protein inhibitor of nitric oxide | Mus musculus |
| | | | | synthase | |
| 454 | BC008106 | 478 | 100 | dynein, cytoplasmic, light | Mus musculus |
| | | | | polypeptide | |
| 454 | BC034258 | 478 | 100 | dynein, cytoplasmic, light | Mus musculus |
| | | | | polypeptide | |
| 455 | AAH01539 | 1466 | 100 | BC001539 dickkopf (Xenopus | Homo sapiens |
| | | | | laevis) homolog 1 | |
| 455 | AF177394_1 | 1466 | 100 | AF177394 dickkopf-1 | Homo sapiens |
| 455 | AF261158 | 1466 | 100 | dickkopf homolog 1 | Homo sapiens |
| 456 | AAC33279 | 1396 | 100 | AC005559 basigin | Homo sapiens |
| 456 | AAH09040 | 1396 | 100 | BC009040 basigin (OK blood group) | Homo sapiens |
| 456 | AF042855 | 1396 | 100 | EMMPRIN | Homo sapiens |
| 457 | L47125 | 3057 | 100 | glypican | Homo sapiens |
| 457 | L47176 | 3057 | 100 | GTR2-2 gene product | Homo sapiens |
| 457 | Z37987 | 3057 | 100 | MXR7 | Homo sapiens |
| 458 | D83476 | 4306 | 77 | Xtld protein | Xenopus |
| | | | | | laevis |
| 458 | L24755 | 4948 | 92 | bone morphogenetic protein | Mus musculus |
| 458 | U50330 | 5416 | 100 | procollagen C-proteinase | Homo sapiens |
| 459 | AAH01287 | 669 | 100 | BC001287 histidine triad | Homo sapiens |
| | | | | nucleotide-binding protein | |
| 459 | AAH07090 | 669 | 100 | BC007090 histidine triad | Homo sapiens |
| | | | | nucleotide-binding protein | |
| 459 | U51004 | 669 | 100 | protein kinase C inhibitor | Homo sapiens |
| 460 | AB003306 | 1280 | 94 | PSMB5 | Mus musculus |
| 460 | AF060091_1 | 1280 | 94 | AF060091 proteasome subunit X | Mus musculus |
| 460 | X95586 | 1370 | 100 | proteasome | Homo sapiens |
| 461 | AF102848_1 | 2178 | 100 | AF102848 keratin 23 | Homo sapiens |
| 461 | AK002047 | 2151 | 98 | unnamed protein product | Homo sapiens |
| 461 | BC028356 | 2155 | 99 | type I intermediate filament | Homo sapiens |
| | | | | cytokeratin | |
| 462 | AAH00097 | 3483 | 100 | BC000097 transforming growth | Homo sapiens |
| | | | | factor, beta-induced, 68 kD | |
| 462 | AAH04972 | 3483 | 100 | BC004972 transforming growth | Homo sapiens |
| | | | | factor, beta-induced, 68 kD | |
| 462 | AY149344 | 3483 | 100 | transforming growth factor, beta- | Homo sapiens |
| | | | | induced, 68 kDa | |
| 463 | AF414110_1 | 635 | 100 | AF414110 histone variant H2A.F/Z | Danio rerio |
| 463 | AF414111_1 | 635 | 100 | AF414111 histone variant H2A.F/Z | Danio rerio |
| 463 | V00414 | 635 | 100 | histone H2A | Gallus |
| | | | | | gallus |
| 464 | BC011457 | 957 | 100 | Unknown (protein for MGC: 7976) | Mus musculus |
| 464 | BC019761 | 957 | 100 | putative membrane protein | Mus musculus |
| 464 | BC020098 | 957 | 100 | putative membrane protein | Mus musculus |
| 465 | AAH00140 | 3579 | 100 | BC000140 propionyl Coenzyme A | Homo sapiens |
| | | | | carboxylase, alpha polypeptide | |
| 465 | AF385926_1 | 3579 | 100 | AF385926 propionyl-CoA | Homo sapiens |
| | | | | carboxylase alpha subunit | |
| 465 | AY035808 | 3579 | 100 | propionyl-CoA carboxylase alpha | Homo sapiens |
| | | | | polypeptide precursor | |
| 466 | AF372216_1 | 562 | 99 | AF372216 tropomyosin alpha | Rattus |
| | | | | isoform | norvegicus |
| 466 | M19267 | 565 | 100 | tropomyosin | Homo sapiens |
| 466 | M19715 | 565 | 100 | skeletal muscle tropomyosin | Homo sapiens |
| 467 | AAH00191 | 704 | 100 | BC000191 hypothetical protein | Homo sapiens |
| 467 | AAH14329 | 704 | 100 | BC014329 Unknown (protein for | Homo sapiens |
| | | | | MGC: 22862) | |
| 467 | AF212248_1 | 704 | 100 | AF212248 CDA09 | Homo sapiens |
| 468 | AAH00255 | 783 | 100 | BC000255 Unknown (protein for | Homo sapiens |
| | | | | MGC: 2495) | |
| 468 | AF320778_1 | 783 | 100 | AF320778 cervical cancer oncogene 3 | Homo sapiens |
| 468 | AY032594 | 783 | 100 | hepatitis C virus core-binding | Homo sapiens |
| | | | | protein 6 | |
| 469 | AAH00271 | 799 | 100 | BC000271 Unknown (protein for | Homo sapiens |
| | | | | MGC: 3204) | |
| 469 | AAH01434 | 799 | 100 | BC001434 Unknown (protein for | Homo sapiens |
| | | | | MGC: 2477) | |
| 469 | AK007390 | 449 | 82 | unnamed protein product | Mus musculus |
| 470 | AAH00421 | 1243 | 100 | BC000421 lysosomal-associated | Homo sapiens |
| | | | | protein transmembrane 4 alpha | |
| 470 | AAH03158 | 1240 | 99 | BC003158 lysosomal-associated | Homo sapiens |
| | | | | protein transmembrane 4 alpha | |
| 470 | D14696 | 1243 | 100 | KIAA0108 | Homo sapiens |
| 471 | AAH00461 | 1738 | 100 | BC000461 eukaryotic translation | Homo sapiens |
| | | | | initiation factor 2, subunit 2 | |
| | | | | (beta, 38 kD) | |
| 471 | AAH00934 | 1735 | 99 | BC000934 eukaryotic translation | Homo sapiens |
| | | | | initiation factor 2, subunit 2 | |
| | | | | (beta, 38 kD) | |
| 471 | AL031668 | 1738 | 100 | dJ64K7.2 (eukaryotic translation | Homo sapiens |
| | | | | initiation factor 2, subunit 2 | |
| | | | | (beta, 38 kD)) | |
| 472 | AAH00466 | 1059 | 100 | BC000466 NADH dehydrogenase | Homo sapiens |
| | | | | (ubiquinone) 1 beta subcomplex, 8 | |
| | | | | (19 kD, ASHI) | |
| 472 | AAH19276 | 1059 | 100 | BC019276 NADH dehydrogenase | Homo sapiens |
| | | | | (ubiquinone) 1 beta subcomplex, 8 | |
| | | | | (19 kD, ASHI) | |
| 472 | AF044958 | 1059 | 100 | NADH: ubiquinone oxidoreductase | Homo sapiens |
| | | | | ASHI subunit | |
| 473 | AAH00490 | 1821 | 100 | BC000490 eukaryotic translation | Homo sapiens |
| | | | | initiation factor 3, subunit 5 | |
| | | | | (epsilon, 47 kD) | |
| 473 | AK095574 | 1774 | 98 | unnamed protein product | Homo sapiens |
| 473 | U94855 | 1821 | 100 | translation initiation factor 3 | Homo sapiens |
| | | | | 47 kDa subunit | |
| 474 | AAH00502 | 970 | 100 | BC000502 ribosomal protein L17 | Homo sapiens |
| 474 | AAH17831 | 970 | 100 | BC017831 ribosomal protein L17 | Homo sapiens |
| 474 | X53777 | 970 | 100 | putative ribosomal protein (AA 1-184) | Homo sapiens |
| 475 | AAH00505 | 808 | 100 | BC000505 microsomal glutathione | Homo sapiens |
| | | | | S-transferase 3 | |
| 475 | AAH03034 | 808 | 100 | BC003034 microsomal glutathione | Homo sapiens |
| | | | | S-transferase 3 | |
| 475 | AAH05964 | 808 | 100 | BC005964 microsomal glutathione | Homo sapiens |
| | | | | S-transferase 3 | |
| 476 | AAH00509 | 1425 | 100 | BC000509 proteasome (prosome, | Homo sapiens |
| | | | | macropain) subunit, beta type, 7 | |
| 476 | AAH17116 | 1393 | 100 | BC017116 proteasome (prosome, | Homo sapiens |
| | | | | macropain) subunit, beta type, 7 | |
| 476 | D38048 | 1421 | 99 | proteasome subunit z | Homo sapiens |
| 477 | BC033015 | 4539 | 100 | RAS p21 protein activator (GTPase | Homo sapiens |
| | | | | activating protein) 1 | |
| 477 | M23379 | 4539 | 100 | GTPase-activating protein | Homo sapiens |
| 477 | M23612 | 4555 | 100 | GTPase-activating protein | Homo sapiens |
| 478 | BC003860 | 2254 | 100 | protease (prosome, macropain) 26S | Mus musculus |
| | | | | subunit, ATPase 1 | |
| 478 | D50696 | 2254 | 100 | proteasomal ATPase (S4) | Rattus |
| | | | | | norvegicus |
| 478 | U39302 | 2254 | 100 | P26s4 | Mus musculus |
| 479 | AAH00522 | 2131 | 100 | BC000522 Similar to serine (or | Homo sapiens |
| | | | | cysteine) proteinase inhibitor, | |
| | | | | clade F (alpha-2 antiplasmin, | |
| | | | | pigment epithelium derived | |
| | | | | factor). member 1 | |
| 479 | AF400442_1 | 2125 | 99 | AF400442 pigment epithelium- | Homo sapiens |
| | | | | derived factor | |
| 479 | M76979 | 2119 | 99 | pigment epithelial- | Homo sapiens |
| | | | | differentiating factor | |
| 480 | AAH00529 | 1616 | 100 | BC000529 prostate differentiation | Homo sapiens |
| | | | | factor | |
| 480 | AAH08962 | 1616 | 100 | BC008962 Unknown (protein for | Homo sapiens |
| | | | | MGC: 4145) | |
| 480 | AF003934 | 1613 | 99 | prostate differentiation factor | Homo sapiens |
| 481 | AAH00548 | 806 | 100 | BC000548 receptor (calcitonin) | Homo sapiens |
| | | | | activity modifying protein 1 | |
| 481 | AF181550_1 | 596 | 71 | AF181550 receptor activity | Rattus |
| | | | | modifying protein 1 | norvegicus |
| 481 | AJ001014 | 806 | 100 | RAMP1 | Homo sapiens |
| 482 | AAH00554 | 1409 | 100 | BC000554 Similar to integral | Homo sapiens |
| | | | | membrane protein 2B | |
| 482 | AF136973_1 | 1409 | 100 | AF136973 putative transmembrane | Homo sapiens |
| | | | | protein E3-16 | |
| 482 | AF152462_1 | 1409 | 100 | AF152462 transmembrane protein | Homo sapiens |
| | | | | BRI | |
| 483 | AAH00601 | 479 | 100 | BC000601 DKFZP564K247 protein | Homo sapiens |
| 483 | AAH09583 | 479 | 100 | BC009583 DKFZP564K247 protein | Homo sapiens |
| 483 | AAH09594 | 479 | 100 | BC009594 DKFZP564K247 protein | Homo sapiens |
| 484 | AAH00691 | 906 | 100 | BC000691 brain specific protein | Homo sapiens |
| 484 | AF132972_1 | 902 | 99 | AF132972 CGI-38 protein | Homo sapiens |
| 484 | BC010788 | 888 | 96 | RIKEN cDNA 2700055K07 gene | Mus musculus |
| 485 | AAH00720 | 798 | 100 | BC000720 ubiquitously-expressed | Homo sapiens |
| | | | | transcript | |
| 485 | AAH08890 | 798 | 100 | BC008890 ubiquitously-expressed | Homo sapiens |
| | | | | transcript | |
| 485 | AF092737_1 | 798 | 100 | AF092737 ubiquitously expressed | Homo sapiens |
| | | | | transcript | |
| 486 | AAH01016 | 958 | 100 | BC001016 NADH dehydrogenase | Homo sapiens |
| | | | | (ubiquinone) 1 alpha subcomplex, | |
| | | | | 8 (19 kD, PGIV) | |
| 486 | AF044953_1 | 958 | 100 | AF044953 NADH: ubiquinone | Homo sapiens |
| | | | | oxidoreductase PGIV subunit | |
| 486 | X59697 | 866 | 88 | 19 kDa subunit of NADH: ubiquinone | Bos taurus |
| | | | | oxidoreductase complex (complex | |
| | | | | I) | |
| 487 | AAH01066 | 763 | 100 | BC001066 hypothetical protein | Homo sapiens |
| 487 | AAH21986 | 763 | 100 | BC021986 mitochondrial ribosomal | Homo sapiens |
| | | | | protein L27 | |
| 487 | AB049647 | 763 | 100 | mitochondrial ribosomal protein | Homo sapiens |
| | | | | L27 (L27mt) | |
| 488 | AAH01101 | 2975 | 100 | BC001101 HSPC025 | Homo sapiens |
| 488 | AAH07510 | 2975 | 100 | BC007510 HSPC025 | Homo sapiens |
| 488 | BC029265 | 2975 | 100 | eukaryotic translation initiation | Homo sapiens |
| | | | | factor 3, subunit 6 interacting | |
| | | | | protein | |
| 489 | AAH01150 | 2803 | 100 | BC001150 metalloprotease 1 | Homo sapiens |
| | | | | (pitrilysin family) | |
| 489 | AAH05025 | 2802 | 99 | BC005025 Similar to | Homo sapiens |
| | | | | metalloprotease 1 (pitrilysin | |
| | | | | family) | |
| 489 | AB029027 | 2803 | 100 | KIAA1104 protein | Homo sapiens |
| 490 | AAH01288 | 2064 | 100 | BC001288 Similar to decay | Homo sapiens |
| | | | | accelerating factor for | |
| | | | | complement (CD55, Cromer blood | |
| | | | | group system) | |
| 490 | M30142 | 2064 | 100 | decay-accelerating factor A | Homo sapiens |
| 490 | M31516 | 2064 | 100 | decay-accelerating factor | Homo sapiens |
| 491 | AAH01387 | 833 | 100 | BC001387 similar to rat HREV107 | Homo sapiens |
| 491 | AB030814 | 833 | 100 | H-REV107 protein homolog | Homo sapiens |
| 491 | AF317086 | 833 | 100 | HREV107-3 | Homo sapiens |
| 492 | AAH01420 | 800 | 100 | BC001420 HN1 protein | Homo sapiens |
| 492 | AF177862_1 | 800 | 100 | AF177862 HN1 protein | Homo sapiens |
| 492 | AF348672 | 800 | 100 | hematological and neurological | Homo sapiens |
| | | | | expressed 1 protein | |
| 493 | AAH01426 | 482 | 100 | BC001426 Similar to ubiquinol- | Homo sapiens |
| | | | | cytochrome c reductase hinge | |
| | | | | protein | |
| 493 | AAH01934 | 482 | 100 | BC001934 Similar to ubiquinol- | Homo sapiens |
| | | | | cytochrome c reductase hinge | |
| | | | | protein | |
| 493 | AAH15177 | 482 | 100 | BC015177 Unknown (protein for | Homo sapiens |
| | | | | MGC: 10149) | |
| 494 | AAH21173 | 429 | 100 | BC021173 normal mucosa of | Homo sapiens |
| | | | | esophagus specific 1 | |
| 494 | AB026707 | 429 | 100 | FOAP-11 protein | Homo sapiens |
| 494 | AF228422_1 | 429 | 100 | AF228422 normal mucosa of | Homo sapiens |
| | | | | esophagus specific 1 | |
| 495 | AAH15173 | 608 | 100 | BC015173 ribosomal protein, | Homo sapiens |
| | | | | large, P0 | |
| 495 | AAH15690 | 608 | 100 | BC015690 ribosomal protein, | Homo sapiens |
| | | | | large, P0 | |
| 495 | AF274958_1 | 737 | 100 | AF274958 PNAS-101 | Homo sapiens |
| 496 | AAH14908 | 1453 | 100 | BC014908 stem-loop (histone) | Homo sapiens |
| | | | | binding protein | |
| 496 | AAH15703 | 1453 | 100 | BC015703 stem-loop (histone) | Homo sapiens |
| | | | | binding protein | |
| 496 | Z71188 | 1453 | 100 | histone RNA hairpin-binding | Homo sapiens |
| | | | | protein | |
| 497 | AB000491 | 2046 | 100 | proteasome p45/SUG | Rattus |
| | | | | | norvegicus |
| 497 | D83521 | 2046 | 100 | proteasomal ATPase (rat SUG1) | Rattus |
| | | | | | norvegicus |
| 497 | Z54219 | 2046 | 100 | mSUG1 protein | Mus musculus |
| 498 | AK075215 | 831 | 100 | unnamed protein product | Homo sapiens |
| 499 | AAH02481 | 480 | 100 | BC002481 HSPC162 protein | Homo sapiens |
| 499 | AF132750_1 | 480 | 100 | AF132750 bithoraxoid-like protein | Homo sapiens |
| 499 | AF178431_1 | 480 | 100 | AF178431 BITH | Homo sapiens |
| 500 | AAH02559 | 3087 | 100 | BC002559 high-glucose-regulated | Homo sapiens |
| | | | | protein 8 | |
| 500 | AK083882 | 3077 | 99 | unnamed protein product | Mus musculus |
| 500 | BC014797 | 3077 | 99 | Unknown (protein for MGC: 11691) | Mus musculus |
| 501 | AAH02589 | 2230 | 100 | BC002589 proteasome (prosome, | Homo sapiens |
| | | | | macropain) 26S subunit, ATPase, 2 | |
| 501 | BC005462 | 2226 | 99 | proteasome (prosome, macropain) | Mus musculus |
| | | | | 26S subunit, ATPase 2 | |
| 501 | D11094 | 2230 | 100 | MSS1 protein | Homo sapiens |
| 502 | AAH02634 | 1769 | 100 | BC002634 Unknown (protein for | Homo sapiens |
| | | | | MGC: 4272) | |
| 502 | AK003354 | 1732 | 96 | unnamed protein product | Mus musculus |
| 502 | BC002128 | 1725 | 96 | Unknown (protein for MGC: 6737) | Mus musculus |
| 503 | AAH02803 | 843 | 100 | BC002803 hypothetical protein | Homo sapiens |
| 503 | AF151072_1 | 843 | 100 | AF151072 HSPC238 | Homo sapiens |
| 503 | BC005559 | 711 | 78 | RIKEN cDNA 2500002L14 gene | Mus musculus |
| 504 | AAH02911 | 856 | 100 | BC002911 Unknown (protein for | Homo sapiens |
| | | | | MGC: 11276) | |
| 504 | AK003237 | 722 | 84 | unnamed protein product | Mus musculus |
| 504 | BC031732 | 722 | 84 | DNA segment, Chr 7, Wayne State | Mus musculus |
| | | | | University 86, expressed | |
| 505 | AAH02945 | 2197 | 100 | BC002945 Similar to hypothetical | Homo sapiens |
| | | | | protein FLJ10101 | |
| 505 | AAH21095 | 1290 | 100 | BC021095 Unknown (protein for | Homo sapiens |
| | | | | MGC: 31800) | |
| 505 | AK027586 | 1290 | 100 | unnamed protein product | Homo sapiens |
| 506 | AAH02954 | 2552 | 100 | BC002954 UDP-glucose | Homo sapiens |
| | | | | pyrophosphorylase 2 | |
| 506 | BC023810 | 2524 | 98 | UDP-glucose pyrophosphorylase 2 | Mus musculus |
| 506 | U27460 | 2542 | 99 | uridine diphosphoglucose | Homo sapiens |
| | | | | pyrophosphorylase | |
| 507 | AAH03005 | 886 | 100 | BC003005 unactive progesterone | Homo sapiens |
| | | | | receptor, 23 kD | |
| 507 | BC003708 | 876 | 98 | telomerase binding protein, p23 | Mus musculus |
| 507 | L24804 | 886 | 100 | p23 | Homo sapiens |
| 508 | AAH03056 | 2132 | 100 | BC003056 HSPC028 protein | Homo sapiens |
| 508 | AAH08453 | 2132 | 100 | BC008453 HSPC028 protein | Homo sapiens |
| 508 | AAH09597 | 2132 | 100 | BC009597 HSPC028 protein | Homo sapiens |
| 510 | AAH01773 | 591 | 100 | BC001773 Similar to ribosomal | Homo sapiens |
| | | | | protein L34 | |
| 510 | AB061832 | 591 | 100 | ribosomal protein L34 | Homo sapiens |
| 510 | BC028517 | 587 | 99 | Unknown (protein for MGC: 41239) | Mus musculus |
| 511 | AAH01882 | 533 | 100 | BC001882 Similar to ribosomal | Homo sapiens |
| | | | | protein L5 | |
| 511 | D10737 | 512 | 100 | ribosomal protein L5 | Gallus |
| | | | | | gallus |
| 511 | X57016 | 512 | 100 | ribosomal protein L5 | Gallus |
| | | | | | gallus |
| 512 | AAH01884 | 589 | 100 | BC001884 NADH dehydrogenase | Homo sapiens |
| | | | | (ubiquinone) Fe-S protein 5 | |
| | | | | (15 kD) (NADH-coenzyme Q | |
| | | | | reductase) | |
| 512 | AF020352 | 589 | 100 | NADH: ubiquinone oxidoreductase 15 kDa | Homo sapiens |
| | | | | IP subunit | |
| 512 | AF047434 | 589 | 100 | NADH-ubiquinone oxidoreductase | Homo sapiens |
| | | | | 15 kDa subunit; CI-15 protein | |
| 513 | AAH01926 | 2197 | 100 | BC001926 creatine kinase, | Homo sapiens |
| | | | | mitochondrial 1 (ubiquitous) | |
| 513 | AAH06467 | 2197 | 100 | BC006467 creatine kinase, | Homo sapiens |
| | | | | mitochondrial 1 (ubiquitous) | |
| 513 | J04469 | 2197 | 100 | creatine kinase | Homo sapiens |
| 514 | AAH03373 | 763 | 100 | BC003373 prefoldin 5 | Homo sapiens |
| 514 | AB055803 | 763 | 100 | MM-1 alpha | Homo sapiens |
| 514 | D89667 | 763 | 100 | c-myc binding protein | Homo sapiens |
| 515 | AAH05939 | 998 | 100 | BC005939 prostaglandin D2 | Homo sapiens |
| | | | | synthase (21 kD, brain) | |
| 515 | AY026356 | 998 | 100 | prostaglandin D synthase | Homo sapiens |
| 515 | M98539 | 998 | 100 | prostaglandin D2 synthase | Homo sapiens |
| 516 | AAH00045 | 1388 | 100 | BC000045 TONDU | Homo sapiens |
| 516 | AAH03362 | 1388 | 100 | BC003362 TONDU | Homo sapiens |
| 516 | Z97632 | 1388 | 100 | dJ196E23.1.1 (novel protein) | Homo sapiens |
| | | | | (isoform 1) | |
| 517 | AAH03366 | 796 | 100 | BC003366 calcium-regulated heat- | Homo sapiens |
| | | | | stable protein (24 kD) | |
| 517 | AF115345_1 | 787 | 99 | AF115345 calcium-regulated heat | Homo sapiens |
| | | | | stable protein CRHSP-24 | |
| 517 | AF115346_1 | 785 | 97 | AF115346 calcium-regulated heat | Rattus sp. |
| | | | | stable protein CRHSP-24 | |
| 518 | AAH03369 | 581 | 100 | BC003369 ribosomal protein, | Homo sapiens |
| | | | | large, P1 | |
| 518 | AAH07590 | 581 | 100 | BC007590 ribosomal protein, | Homo sapiens |
| | | | | large, P1 | |
| 518 | AB061836 | 581 | 100 | ribosomal protein P1 | Homo sapiens |
| 519 | AAH03377 | 550 | 100 | BC003377 Similar to thioredoxin | Homo sapiens |
| 519 | AF313911_1 | 550 | 100 | AF313911 thioredoxin | Homo sapiens |
| 519 | AY004872 | 550 | 100 | thioredoxin | Homo sapiens |
| 520 | BC032493 | 1086 | 100 | cysteine and glycine-rich protein 1 | Homo sapiens |
| 520 | M33146 | 1086 | 100 | cysteine-rich protein | Homo sapiens |
| 520 | M76378 | 1086 | 100 | cysteine-rich protein | Homo sapiens |
| 521 | AAH03382 | 2620 | 100 | BC003382 sorting nexin 2 | Homo sapiens |
| 521 | AF065482 | 2612 | 99 | sorting nexin 2 | Homo sapiens |
| 521 | AK075929 | 2564 | 98 | unnamed protein product | Mus musculus |
| 522 | AAH03394 | 1485 | 100 | BC003394 heterogeneous nuclear | Homo sapiens |
| | | | | ribonucleoprotein C (C1/C2) | |
| 522 | AAH08364 | 1478 | 99 | BC008364 heterogeneous nuclear | Homo sapiens |
| | | | | ribonucleoprotein C (C1/C2) | |
| 522 | AAH08423 | 1461 | 95 | BC008423 heterogeneous nuclear | Homo sapiens |
| | | | | ribonucleoprotein C (C1/C2) | |
| 523 | AAH03501 | 2155 | 100 | BC003501 Similar to RIKEN cDNA | Homo sapiens |
| | | | | 2310001A20 gene | |
| 523 | AB033767 | 2155 | 100 | brain-selective and closely | Homo sapiens |
| | | | | mapped on the counter allele of | |
| | | | | CMAP in cystatin cluster | |
| 523 | AL035661 | 2155 | 100 | dJ568C11.2 (chromosome 20 open | Homo sapiens |
| | | | | reading frame 3) | |
| 524 | AAH03512 | 3204 | 100 | BC003512 mesothelin | Homo sapiens |
| 524 | AE006464_21 | 3189 | 99 | AE006464 pre-pro-megakarycyte | Homo sapiens |
| | | | | potentiating factor precursor | |
| 524 | D49441 | 3192 | 99 | pre-pro-megakaryocyte | Homo sapiens |
| | | | | potentiating factor | |
| 525 | AAH03077 | 5288 | 100 | BC003077 Similar to ATPase, Na+K+ | Homo sapiens |
| | | | | transporting, alpha 1 polypeptide | |
| 525 | D00099 | 5288 | 100 | Na, K-ATPase alpha-subunit | Homo sapiens |
| 525 | X04297 | 5288 | 100 | ATPase alpha subunit (aa 1-1023) | Homo sapiens |
| 526 | AAH03079 | 786 | 100 | BC003079 16.7 Kd protein | Homo sapiens |
| 526 | AAH15639 | 786 | 100 | BC015639 16.7 Kd protein | Homo sapiens |
| 526 | AF078845 | 786 | 100 | 16.7 Kd protein | Homo sapiens |
| 527 | AAH00161 | 1827 | 100 | BC000161 secretory carrier | Homo sapiens |
| | | | | membrane protein 3 | |
| 527 | AAH05135 | 1827 | 100 | BC005135 secretory carrier | Homo sapiens |
| | | | | membrane protein 3 | |
| 527 | AF005039 | 1817 | 99 | secretory carrier membrane | Homo sapiens |
| | | | | protein | |
| 528 | AAH08704 | 887 | 100 | BC008704 cytochrome c oxidase | Homo sapiens |
| | | | | subunit IV | |
| 528 | AAH21236 | 887 | 100 | BC021236 cytochrome c oxidase | Homo sapiens |
| | | | | subunit IV isoform 1 | |
| 528 | X54802 | 887 | 100 | cytochrome-c oxidase subunit IV | Homo sapiens |
| 529 | AAH00915 | 1731 | 100 | BC000915 PDZ and LIM domain 1 | Homo sapiens |
| | | | | (elfin) | |
| 529 | AAH18755 | 1731 | 100 | BC018755 PDZ and LIM domain 1 | Homo sapiens |
| | | | | (elfin) | |
| 529 | AJ310549 | 1731 | 100 | CLP-36 protein | Homo sapiens |
| 530 | AF236636 | 1302 | 98 | uridine-cytidine kinase 2 | Mus musculus |
| 530 | AF236637 | 1333 | 100 | uridine-cytidine kinase 2 | Homo sapiens |
| 530 | BC023789 | 1302 | 98 | uridine-cytidine kinase 2 | Mus musculus |
| 531 | AAH00176 | 940 | 100 | BC000176 RAP1B, member of RAS | Homo sapiens |
| | | | | oncogene family | |
| 531 | AF493913_1 | 940 | 100 | AF493913 Ras family small GTP | Homo sapiens |
| | | | | binding protein RAP1B | |
| 531 | BC033382 | 952 | 87 | RAP1B, member of RAS oncogene | Mus musculus |
| | | | | family | |
| 532 | D63519 | 870 | 100 | leptin | Homo sapiens |
| 532 | D63710 | 870 | 100 | ob protein | Homo sapiens |
| 532 | U43653 | 870 | 100 | obese protein | Homo sapiens |
| 533 | BC002088 | 637 | 100 | ribosomal protein S25 | Mus musculus |
| 533 | BC027208 | 637 | 100 | ribosomal protein S25 | Mus musculus |
| 533 | X62482 | 637 | 100 | ribosomal protein S25 | Rattus |
| | | | | | Rattus |
| 534 | AAH03662 | 2092 | 100 | BC003662 KIAA0111 gene product | Homo sapiens |
| 534 | AAH04386 | 2092 | 100 | BC004386 KIAA0111 gene product | Homo sapiens |
| 534 | AAH11151 | 2092 | 100 | BC011151 Similar to KIAA0111 gene | Homo sapiens |
| | | | | product | |
| 535 | AF349038_1 | 1780 | 100 | AF349038 TFIID subunit TAFII55 | Homo sapiens |
| 535 | BC032737 | 1780 | 100 | similar to TAF7 RNA polymerase | Homo sapiens |
| | | | | II, TATA box binding protein | |
| | | | | (TBP)-associated factor, 55 kD | |
| 535 | X97999 | 1780 | 100 | transcription factor IID | Homo sapiens |
| 536 | AAH02601 | 1673 | 100 | BC002601 nuclear factor of kappa | Homo sapiens |
| | | | | light polypeptide gene enhancer | |
| | | | | in B-cells inhibitor, alpha | |
| 536 | AAH04983 | 1673 | 100 | BC004983 nuclear factor of kappa | Homo sapiens |
| | | | | light polypeptide gene enhancer | |
| | | | | in B-cells inhibitor, alpha | |
| 536 | AY033600 | 1673 | 100 | NFKBIA | Homo sapiens |
| 537 | AF211480_1 | 2102 | 100 | AF211480 CD001 | Homo sapiens |
| 537 | AK002102 | 2086 | 100 | unnamed protein product | Homo sapiens |
| 537 | AX191505 | 2086 | 100 | unnamed protein product | Homo sapiens |
| 538 | AAH05110 | 4609 | 100 | BC005110 Unknown (protein for | Homo sapiens |
| | | | | MGC: 13217) | |
| 538 | AK049371 | 4473 | 93 | unnamed protein product | Mus musculus |
| 538 | AY044865 | 4609 | 100 | sorting nexin 14 | Homo sapiens |
| 539 | AAH05118 | 592 | 100 | BC005118 Similar to putative | Homo sapiens |
| | | | | translation initiation factor | |
| 539 | AAH08710 | 592 | 100 | BC008710 putative translation | Homo sapiens |
| | | | | initiation factor | |
| 539 | AF083441_1 | 592 | 100 | AF083441 SUI1 isolog | Homo sapiens |
| 540 | AAH05127 | 2190 | 100 | BC005127 adipose differentiation- | Homo sapiens |
| | | | | related protein | |
| 540 | AF443203_1 | 2190 | 100 | AF443203 adipose differentiation- | Homo sapiens |
| | | | | related protein | |
| 540 | AX025098 | 2181 | 99 | unnamed protein product | Homo sapiens |
| 541 | AAH05143 | 964 | 100 | BC005143 vitamin A responsive; | Homo sapiens |
| | | | | cytoskeleton related | |
| 541 | AAH20797 | 964 | 100 | BC020797 vitamin A responsive; | Homo sapiens |
| | | | | cytoskeleton related | |
| 541 | AY102608 | 964 | 100 | JWA protein | Homo sapiens |
| 542 | AAH05228 | 360 | 100 | BC005228 Unknown (protein for | Homo sapiens |
| | | | | MGC: 12250) | |
| 543 | AF072506 | 2871 | 100 | envelope protein precursor | Homo sapiens |
| 543 | AF208161 | 2868 | 99 | syncytin precursor | Homo sapiens |
| 543 | AF513360_1 | 2868 | 99 | AF513360 enverin | Homo sapiens |
| 544 | AAH05322 | 1866 | 100 | BC005322 decorin | Homo sapiens |
| 544 | AF138300 | 1866 | 100 | decorin variant A | Homo sapiens |
| 544 | AF491944_1 | 1866 | 100 | AF491944 decorin | Homo sapiens |
| 545 | AAH01392 | 827 | 100 | BC001392 ribosomal protein S27a | Homo sapiens |
| 545 | D83209 | 827 | 100 | ubiquitin extention protein | Cavia |
| | | | | | porcellus |
| 545 | M24507 | 827 | 100 | ubiquitin | synthetic |
| | | | | | construct |
| 546 | AAH05330 | 1306 | 100 | BC005330 tissue factor pathway | Homo sapiens |
| | | | | inhibitor 2 | |
| 546 | AF217542 | 1306 | 100 | tissue factor pathway inhibitor 2 | Homo sapiens |
| 546 | D29992 | 1306 | 100 | placental protein 5 (PP5) | Homo sapiens |
| 547 | AAH05361 | 1334 | 100 | BC005361 proteasome (prosome, | Homo sapiens |
| | | | | macropain) subunit, alpha type, 4 | |
| 547 | BC022445 | 1334 | 100 | proteasome (prosome, macropain) | Homo sapiens |
| | | | | subunit, alpha type, 4 | |
| 547 | D00763 | 1334 | 100 | proteasome subunit C9 | Homo sapiens |
| 548 | AAH05366 | 1298 | 100 | BC005366 ATP synthase, H+ | Homo sapiens |
| | | | | transporting, mitochondrial F0 | |
| | | | | complex, subunit b, isoform 1 | |
| 548 | AAH16350 | 1298 | 100 | BC016350 Unknown (protein for | Homo sapiens |
| | | | | MGC: 24431) | |
| 548 | AL390195 | 1298 | 100 | bA552M11.3 (ATP synthase, H+ | Homo sapiens |
| | | | | transporting, mitochondrial F0 | |
| | | | | complex, subunit b, isoform 1) | |
| 549 | AAH05390 | 1993 | 100 | BC005390 Unknown (protein for | Homo sapiens |
| | | | | MGC: 12520) | |
| 549 | AF006305 | 1993 | 100 | 26S proteasome regulatory subunit | Homo sapiens |
| 549 | U36395 | 1993 | 100 | conserved ATPase domain protein | Spermophilus |
| | | | | 44 | tridecemlineatus |
| 550 | AAH10370 | 1800 | 99 | BC010370 Putative prostate cancer | Homo sapiens |
| | | | | tumor suppressor | |
| 550 | U42349 | 1826 | 100 | 39 kDa encoded by N33 | Homo sapiens |
| 550 | U42360 | 1808 | 99 | N33 protein form 1 | Homo sapiens |
| 551 | AAH05839 | 1492 | 100 | BC005839 follistatin-like 3 | Homo sapiens |
| | | | | (secreted glycoprotein) | |
| 551 | BC033119 | 1492 | 100 | follistatin-like 3 (secreted | Homo sapiens |
| | | | | glycoprotein) | |
| 551 | U76702 | 1492 | 100 | follistatin-related protein FLRG | Homo sapiens |
| 552 | AAH05354 | 564 | 100 | BC005354 ribosomal protein, large | Homo sapiens |
| | | | | P2 | |
| 552 | AAH05920 | 564 | 100 | BC005920 ribosomal protein, large | Homo sapiens |
| | | | | P2 | |
| 552 | AAH07573 | 564 | 100 | BC007573 ribosomal protein, large | Homo sapiens |
| | | | | P2 | |
| 553 | AAH17660 | 414 | 100 | BC017660 Unknown (protein for | Homo sapiens |
| | | | | MGC: 14608) | |
| 553 | AJ249731 | 414 | 100 | putative G8.2 protein | Homo sapiens |
| 553 | AJ249732 | 307 | 79 | G8 protein | Homo sapiens |
| 554 | AAH05966 | 647 | 100 | BC005966 ring finger protein 7 | Homo sapiens |
| 554 | AAH08627 | 647 | 100 | BC008627 ring finger protein 7 | Homo sapiens |
| 554 | AF092878_1 | 647 | 100 | AF092878 zinc RING finger protein | Homo sapiens |
| | | | | SAG | |
| 555 | AAH05975 | 1163 | 100 | BC005975 calcyclin binding | Homo sapiens |
| | | | | protein | |
| 555 | AF314752_1 | 1163 | 100 | AF314752 calcyclin binding | Homo sapiens |
| | | | | protein | |
| 555 | BC022352 | 1163 | 100 | Siah-interacting protein | Homo sapiens |
| 556 | AAH06337 | 739 | 100 | BC006337 Unknown (protein for | Homo sapiens |
| | | | | MGC: 12798) | |
| 556 | M37194 | 739 | 100 | clathrin-associated protein 17 | Rattus |
| | | | | | norvegicus |
| 556 | X97074 | 729 | 98 | clathrin-associated protein | Homo sapiens |
| 557 | AAH01928 | 3365 | 100 | BC001928 protein disulfide | Homo sapiens |
| | | | | isomerase related protein | |
| | | | | (calcium-binding protein, | |
| | | | | intestinal-related) | |
| 557 | AAH06344 | 3365 | 100 | BC006344 protein disulfide | Homo sapiens |
| | | | | isomerase related protein | |
| | | | | (calcium-binding protein, | |
| | | | | intestinal-related) | |
| 557 | AAH11754 | 3365 | 100 | BC011754 Similar to protein | Homo sapiens |
| | | | | disulfide isomerase related | |
| | | | | protein (calcium-binding protein, | |
| | | | | intestinal-related) | |
| 558 | AF453478_1 | 1155 | 100 | AF453478 phosphopantetheine | Homo sapiens |
| | | | | adenylyltransferase/ | |
| | | | | dephosphocoenzyme A kinase | |
| 558 | AY094602 | 1155 | 100 | bifunctional phosphopantetheine | Homo sapiens |
| | | | | adenylyl transferase/dephospho | |
| | | | | CoA kinase | |
| 558 | BC020985 | 1155 | 100 | Unknown (protein for MGC: 9724) | Homo sapiens |
| 559 | AAH06393 | 2797 | 100 | BC006393 Similar to | Homo sapiens |
| | | | | carboxypeptidase Z | |
| 559 | AF017638 | 2419 | 86 | carboxypeptidase Z | Rattus |
| | | | | | norvegicus |
| 559 | U83411 | 2787 | 99 | carboxypeptidase Z precursor | Homo sapiens |
| 560 | AF126110_1 | 3913 | 100 | AF126110 fibulin-1 isoform D | Homo sapiens |
| | | | | precursor | |
| 560 | U01244 | 3913 | 100 | fibulin-1D | Homo sapiens |
| 560 | X70854 | 3392 | 84 | BM-90/fibulin | Mus musculus |
| 561 | AAH02343 | 2494 | 100 | BC002343 Similar to nucleolin | Homo sapiens |
| 561 | AAH06494 | 2494 | 100 | BC006494 Unknown (protein for | Homo sapiens |
| | | | | MGC: 1440) | |
| 561 | AAH06516 | 2494 | 100 | BC006516 Unknown (protein for | Homo sapiens |
| | | | | MGC: 3588) | |
| 562 | AAH20515 | 772 | 100 | BC020515 ribosomal protein S14 | Homo sapiens |
| 562 | M11241 | 772 | 100 | ribosomal protein S14 | Cricetulus |
| | | | | | griseus |
| 562 | M35008 | 772 | 100 | ribosomal protein S14 | Cricetulus |
| | | | | | griseus |
| 563 | AAH08926 | 645 | 100 | BC008926 ribosomal protein L29 | Homo sapiens |
| 563 | U10248 | 645 | 100 | ribosomal protein L29 | Homo sapiens |
| 563 | U49083 | 645 | 100 | HIP | Homo sapiens |
| 564 | AAH06791 | 1105 | 100 | BC006791 ribosomal protein L10a | Homo sapiens |
| 564 | AAH11366 | 1105 | 100 | BC011366 Similar to ribosomal | Homo sapiens |
| | | | | protein L10a | |
| 564 | X93352 | 1105 | 100 | ribosomal protein L10a | Rattus |
| | | | | | norvegicus |
| 565 | AAH01418 | 1132 | 100 | BC001418 Similar to RIKEN cDNA | Homo sapiens |
| | | | | 1810017F10 gene | |
| 565 | AAH13953 | 1132 | 100 | BC013953 Unknown (protein for | Homo sapiens |
| | | | | MGC: 2853) | |
| 565 | AY027543 | 1132 | 100 | beta-casein-like protein | Homo sapiens |
| 566 | AAH07038 | 1762 | 100 | BC007038 lumican | Homo sapiens |
| 566 | BC035997 | 1762 | 100 | lumican | Homo sapiens |
| 566 | U21128 | 1762 | 100 | lumican | Homo sapiens |
| 567 | AAH03609 | 1441 | 100 | BC003609 thioredoxin peroxidase | Homo sapiens |
| | | | | (antioxidant enzyme) | |
| 567 | AAH07107 | 1441 | 100 | BC007107 thioredoxin peroxidase | Homo sapiens |
| | | | | (antioxidant enzyme) | |
| 567 | AAH16770 | 1441 | 100 | BC016770 thioredoxin peroxidase | Homo sapiens |
| | | | | (antioxidant enzyme) | |
| 568 | AAH07282 | 952 | 100 | BC007282 Unknown (protein for | Homo sapiens |
| | | | | MGC: 15626) | |
| 568 | AK014338 | 909 | 97 | unnamed protein product | Mus musculus |
| 568 | M83751 | 937 | 98 | arginine-rich protein | Homo sapiens |
| 569 | AAH07402 | 1005 | 100 | BC007402 apolipoprotein D | Homo sapiens |
| 569 | J02611 | 1005 | 100 | apolipoprotein D precursor | Homo sapiens |
| 569 | M16696 | 1005 | 100 | apolipoprotein D precursor | Homo sapiens |
| 570 | AAH01603 | 849 | 100 | BC001603 Similar to ribosomal | Homo sapiens |
| | | | | protein L21 | |
| 570 | AAH07505 | 849 | 100 | BC007505 Unknown (protein for | Homo sapiens |
| | | | | MGC: 4136) | |
| 570 | X89401 | 849 | 100 | ribosomal protein L21 | Homo sapiens |
| 571 | AAH07507 | 605 | 100 | BC007507 ribosomal protein S20 | Homo sapiens |
| 571 | BC011323 | 605 | 100 | Similar to ribosomal protein S20 | Mus musculus |
| 571 | X51537 | 605 | 100 | ribosomal protein S20 (AA 1-119) | Rattus |
| | | | | | Rattus |
| 572 | AAH14459 | 791 | 100 | BC014459 Similar to ribosomal | Homo sapiens |
| | | | | protein L23a | |
| 572 | BC029892 | 791 | 100 | ribosomal protein L23a | Mus musculus |
| 572 | X65228 | 791 | 100 | ribosomal protein L23a | Rattus |
| | | | | | Rattus |
| 573 | AJ420896 | 2114 | 99 | SPPL2a protein | Homo sapiens |
| 573 | AK027446 | 2126 | 100 | unnamed protein product | Homo sapiens |
| 573 | BC025740 | 2114 | 99 | Similar to hypothetical protein | Homo sapiens |
| | | | | FLJ14540 | |
| 574 | AF036548 | 554 | 82 | RGC-32 | Rattus |
| | | | | | norvegicus |
| 574 | AF036549_1 | 569 | 100 | AF036549 RGC32 | Homo sapiens |
| 574 | AL354833 | 607 | 91 | bA157L14.2 (RGC32, a novel gene | Homo sapiens |
| | | | | induced by complement activation | |
| | | | | in oligodendrocytes) | |
| 575 | AAH01024 | 2749 | 99 | BC001024 putative nucleotide | Homo sapiens |
| | | | | binding protein, estradiol- | |
| | | | | induced | |
| 575 | AK027514 | 2756 | 100 | unnamed protein product | Homo sapiens |
| 575 | AK027516 | 2753 | 99 | unnamed protein product | Homo sapiens |
| 576 | AAH07834 | 563 | 100 | BC007834 Unknown (protein for | Homo sapiens |
| | | | | MGC: 14141) | |
| 577 | AAH03061 | 2338 | 99 | BC003061 protease, cysteine, 1 | Homo sapiens |
| | | | | (legumain) | |
| 577 | D55696 | 2335 | 99 | cysteine protease | Homo sapiens |
| 577 | Y09862 | 2339 | 100 | legumain | Homo sapiens |
| 578 | AAH08081 | 1095 | 100 | BC008081 Similar to KDEL (Lys- | Homo sapiens |
| | | | | Asp-Glu-Leu) endoplasmic | |
| | | | | reticulum protein retention | |
| | | | | receptor 2 | |
| 578 | AAH14568 | 1095 | 100 | BC014568 Similar to KDEL (Lys- | Homo sapiens |
| | | | | Asp-Glu-Leu) endoplasmic | |
| | | | | reticulum protein retention | |
| | | | | receptor 2 | |
| 578 | X63745 | 1095 | 100 | KDEL receptor | Homo sapiens |
| 579 | AF004877 | 7574 | 99 | pro-alpha 2(I) collagen | Homo sapiens |
| 579 | J03464 | 7554 | 99 | pre-pro-alpha-2 type I collagen | Homo sapiens |
| 579 | Z74616 | 7596 | 100 | prepro-alpha2(I) collagen | Homo sapiens |
| 580 | AAH08188 | 946 | 100 | BC008188 Unknown (protein for | Homo sapiens |
| | | | | MGC: 5243) | |
| 580 | AF021819 | 946 | 100 | RNA-binding protein regulatory | Homo sapiens |
| | | | | subunit | |
| 580 | AL034417 | 946 | 100 | bK215D11.1 (RNA-binding protein | Homo sapiens |
| | | | | regulatory subunit) | |
| 581 | AAH08283 | 594 | 100 | BC008283 cholecystokinin | Homo sapiens |
| 581 | L00354 | 594 | 100 | cholecystokinin | Homo sapiens |
| 581 | M60458 | 569 | 95 | cholecystokinin | Macaca |
| | | | | | fascicularis |
| 582 | AAH06794 | 693 | 99 | BC006794 Similar to interferon | Homo sapiens |
| | | | | induced transmembrane protein 3 | |
| | | | | (1-8U) | |
| 582 | AAH08417 | 698 | 100 | BC008417 Similar to interferon | Homo sapiens |
| | | | | induced transmembrane protein 3 | |
| | | | | (1-8U) | |
| 582 | BC022439 | 698 | 100 | interferon induced transmembrane | Homo sapiens |
| | | | | protein 3 (1-8U) | |
| 583 | AAH05993 | 1421 | 100 | BC005993 Unknown (protein for | Homo sapiens |
| | | | | MGC: 14796) | |
| 583 | AAH08691 | 1421 | 100 | BC008691 Unknown (protein for | Homo sapiens |
| | | | | MGC: 8886) | |
| 583 | AAH15644 | 1421 | 100 | BC015644 Ras suppressor protein 1 | Homo sapiens |
| 584 | AAH08938 | 1942 | 100 | BC008938 Similar to | Homo sapiens |
| | | | | histocompatibility 13 | |
| 584 | AAH08959 | 1942 | 100 | BC008959 Similar to | Homo sapiens |
| | | | | histocompatibility 13 | |
| 584 | AF483215_1 | 1934 | 99 | AF483215 minor histocompatibility | Homo sapiens |
| | | | | antigen H13 isoform 1 | |
| 585 | AAH16320 | 2150 | 100 | BC016320 cathepsin D (lysosomal | Homo sapiens |
| | | | | aspartyl protease) | |
| 585 | M11233 | 2150 | 100 | preprocathepsin D | Homo sapiens |
| 585 | X05344 | 2150 | 100 | precursor polypeptide (AA −20 to | Homo sapiens |
| | | | | 392) | |
| 586 | AAH08983 | 1364 | 100 | BC008983 complement component 1, | Homo sapiens |
| | | | | q subcomponent, beta polypeptide | |
| 586 | X03084 | 1240 | 100 | C1q B-chain precursor | Homo sapiens |
| 586 | X16874 | 1075 | 79 | precursor polypeptide (AA −25 to | Mus musculus |
| | | | | 228) | |
| 587 | AAH09177 | 1014 | 100 | BC009177 ras homolog gene family, | Homo sapiens |
| | | | | member C | |
| 587 | AF498972_1 | 1014 | 100 | AF498972 small GTP binding | Homo sapiens |
| | | | | protein RhoC | |
| 587 | BC004627 | 1014 | 100 | ras homolog gene family, member C | Mus musculus |
| 588 | AAH09200 | 1068 | 100 | BC009200 Rho GDP dissociation | Homo sapiens |
| | | | | inhibitor (GDI) beta | |
| 588 | AF498927_1 | 1068 | 100 | AF498927 Rho GDP dissociation | Homo sapiens |
| | | | | inhibitor beta | |
| 588 | X69549 | 1068 | 100 | Human rho GDP-dissociation | Homo sapiens |
| | | | | Inhibitor 2(IEF 8120) | |
| 589 | AAH09201 | 1137 | 100 | BC009201 clathrin, light | Homo sapiens |
| | | | | polypeptide (Lca) | |
| 589 | AAH19287 | 1137 | 100 | BC019287 clathrin, light | Homo sapiens |
| | | | | polypeptide (Lca) | |
| 589 | AL158830 | 1137 | 100 | bA421H8.4.2 (clathrin, light | Homo sapiens |
| | | | | polypeptide (LCA)) | |
| 590 | AAH09232 | 596 | 100 | BC009232 Similar to G antigen 8 | Homo sapiens |
| 590 | AJ318880 | 596 | 100 | XAGE-2 protein | Homo sapiens |
| 590 | AJ318891 | 596 | 100 | XAGE-2 protein | Homo sapiens |
| 591 | AAH03378 | 1149 | 100 | BC003378 high-mobility group | Homo sapiens |
| | | | | (nonhistone chromosomal) protein 1 | |
| 591 | BC030981 | 1149 | 100 | high-mobility group (nonhistone | Homo sapiens |
| | | | | chromosomal) protein 1 | |
| 591 | X12597 | 1149 | 100 | HMG-1 protein (AA 1-215) | Homo sapiens |
| 592 | AB002368 | 5586 | 100 | KIAA0370 | Homo sapiens |
| 592 | AY026388 | 5765 | 100 | ran binding protein RanBP20 | Homo sapiens |
| 592 | AY029528 | 5663 | 97 | RANBP20 | Mus musculus |
| 593 | AAH09799 | 1308 | 100 | BC009799 amphiregulin | Homo sapiens |
| | | | | (schwannoma-derived growth | |
| | | | | factor) | |
| 593 | M30703 | 1308 | 100 | amphiregulin | Homo sapiens |
| 593 | M30704 | 1308 | 100 | amphiregulin | Homo sapiens |
| 594 | AAH09869 | 511 | 100 | BC009869 Unknown (protein for | Homo sapiens |
| | | | | MGC: 16406) | |
| 594 | AAH15491 | 511 | 100 | BC015491 Unknown (protein for | Homo sapiens |
| | | | | MGC: 8965) | |
| 594 | BC022326 | 511 | 100 | Unknown (protein for MGC: 22741) | Homo sapiens |
| 595 | AAH09898 | 1921 | 100 | BC009898 spermine synthase | Homo sapiens |
| 595 | AD001528 | 1921 | 100 | spermidine aminopropyltransferase | Homo sapiens |
| 595 | Z49099 | 1916 | 100 | spermine synthase | Homo sapiens |
| 596 | AL365410 | 1055 | 100 | hypothetical protein | Homo sapiens |
| 596 | L32162 | 294 | 70 | transcription factor | Homo sapiens |
| 597 | AAH10016 | 1542 | 100 | BC010016 Similar to CD47 antigen | Homo sapiens |
| | | | | (Rh-related antigen, integrin- | |
| | | | | associated signal transducer) | |
| 597 | AAH12884 | 1542 | 100 | BC012884 Unknown (protein for | Homo sapiens |
| | | | | MGC: 9240) | |
| 597 | Z25521 | 1542 | 100 | integrin associated protein | Homo sapiens |
| 598 | AAH10055 | 784 | 100 | BC010055 hypothetical protein | Homo sapiens |
| | | | | PRO2605 | |
| 598 | AF116721_109 | 642 | 100 | AF116709 PRO2605 | Homo sapiens |
| 599 | AAH04976 | 634 | 100 | BC004976 Unknown (protein for | Homo sapiens |
| | | | | IMAGE: 2958115) | |
| 599 | AAH10076 | 634 | 100 | BC010076 Unknown (protein for | Homo sapiens |
| | | | | MGC: 19576) | |
| 599 | AY061855 | 634 | 100 | mitochondrial ribosomal protein | Homo sapiens |
| | | | | S6 | |
| 600 | AAH10129 | 907 | 100 | BC010129 Similar to hypothetical | Homo sapiens |
| | | | | protein R33729_1 | |
| 600 | AAH14655 | 907 | 100 | BC014655 Unknown (protein for | Homo sapiens |
| | | | | MGC: 20383) | |
| 600 | AL365374 | 894 | 100 | R33729_1 hypothetical protein | Homo sapiens |
| 601 | AB028894 | 824 | 100 | ribosomal protein S11 | Mus musculus |
| 601 | BC012641 | 824 | 100 | ribosomal protein S11 | Mus musculus |
| 601 | U93864 | 824 | 100 | ribosomal protein S11 | Mus musculus |
| 602 | BC002077 | 1058 | 100 | RAB1, member RAS oncogene family | Mus musculus |
| 602 | X15744 | 1058 | 100 | GTP-binding protein | Mus musculus |
| 602 | Y00094 | 1058 | 100 | Ypt1 protein (AA 1-205) | Mus musculus |
| 603 | AAH02947 | 1482 | 100 | BC002947 folate receptor 1 | Homo sapiens |
| | | | | (adult) | |
| 603 | U20391 | 1482 | 100 | folate receptor | Homo sapiens |
| 603 | X62753 | 1482 | 100 | adult folate binding protein | Homo sapiens |
| 604 | U65932 | 3016 | 100 | extracellular matrix protein 1 | Homo sapiens |
| 604 | U65938 | 3016 | 100 | extracellular matrix protein 1 | Homo sapiens |
| 604 | U68186 | 3016 | 100 | extracellular matrix protein 1 | Homo sapiens |
| 605 | AAH10897 | 541 | 100 | BC010897 Similar to JM27 protein | Homo sapiens |
| 605 | AF275258_1 | 541 | 100 | AF275258 PAGE-4 | Homo sapiens |
| 605 | AJ005894 | 541 | 100 | JM27 | Homo sapiens |
| 606 | AAH11171 | 2528 | 100 | BC011171 serine (or cysteine) | Homo sapiens |
| | | | | proteinase inhibitor, clade G (C1 | |
| | | | | inhibitor), member 1 | |
| 606 | AF435921_1 | 2524 | 99 | AF435921 C1 esterase inhibitor | Homo sapiens |
| 606 | X07427 | 2524 | 99 | C1 inhibitor | Homo sapiens |
| 607 | D38112 | 1781 | 93 | cytochrome b | Homo sapiens |
| 607 | U09500 | 1780 | 93 | cytochrome b | Homo sapiens |
| 607 | V00662 | 1785 | 93 | cytochrome B | Homo sapiens |
| 608 | AAH11514 | 696 | 100 | BC011514 Similar to ribosomal | Homo sapiens |
| | | | | protein L32 | |
| 608 | K02060 | 696 | 100 | ribosomal protein L32-3A | Mus musculus |
| 608 | X06483 | 696 | 100 | ribosomal protein L32 | Rattus |
| | | | | | norvegicus |
| 609 | AAH11786 | 645 | 100 | BC011786 Unknown (protein for | Homo sapiens |
| | | | | MGC: 19839) | |
| 609 | AF318382_1 | 645 | 100 | AF318382 unknown | Homo sapiens |
| 609 | X07868 | 456 | 100 | 1.8 kb mRNA (AA 1-84) | Homo sapiens |
| 610 | AAH11792 | 1649 | 100 | BC011792 Unknown (protein for | Homo sapiens |
| | | | | MGC: 19561) | |
| 610 | AAH17408 | 1643 | 99 | BC017408 Unknown (protein for | Homo sapiens |
| | | | | MGC: 27221) | |
| 610 | U16660 | 1603 | 97 | peroxisomal enoyl-CoA hydratase- | Homo sapiens |
| | | | | like protein | |
| 611 | AAH00159 | 2163 | 100 | BC000159 keratin 17 | Homo sapiens |
| 611 | AAH11901 | 2163 | 100 | BC011901 Similar to keratin 17 | Homo sapiens |
| 611 | X62571 | 2163 | 100 | keratin related product | Homo sapiens |
| 612 | AAH12132 | 3933 | 100 | BC012132 Similar to DEAD/H (Asp- | Homo sapiens |
| | | | | Glu-Ala-Asp/His) box polypeptide 1 | |
| 612 | BC010624 | 3872 | 97 | Unknown (protein for MGC: 11570) | Mus musculus |
| 612 | X70649 | 3933 | 100 | member of DEAD box protein family | Homo sapiens |
| 613 | AF157623_1 | 2466 | 100 | AF157623 HTRA serine protease | Homo sapiens |
| 613 | D87258 | 2466 | 100 | serin protease with IGF-binding | Homo sapiens |
| | | | | motif | |
| 613 | Y07921 | 2466 | 100 | novel serine protease, PRSS11 | Homo sapiens |
| 614 | AF242550_1 | 1030 | 100 | AF242550 cellular nucleic acid | Rattus |
| | | | | binding protein | norvegicus |
| 614 | AF389887 | 1030 | 100 | zinc finger protein 9 | Homo sapiens |
| 614 | D45254 | 1030 | 100 | Cellular Nucleic Acid Binding | Rattus |
| | | | | Protein | norvegicus |
| 615 | AAH14277 | 1191 | 100 | BC014277 Similar to tissue | Homo sapiens |
| | | | | inhibitor of metalloproteinase 3 | |
| | | | | (Sorsby fundus dystrophy, | |
| | | | | pseudoinflammatory) | |
| 615 | U33114 | 1191 | 100 | tissue inhibitor of | Homo sapiens |
| | | | | metalloproteinases-3 | |
| 615 | X76227 | 1191 | 100 | tissue inhibitor of | Homo sapiens |
| | | | | metalloproteinases-3 | |
| 616 | AAH12384 | 1017 | 100 | BC012384 Similar to programmed | Homo sapiens |
| | | | | cell death 6 | |
| 616 | AF035606 | 1017 | 100 | calcium binding protein | Homo sapiens |
| 616 | U58773_1 | 1017 | 100 | U58773 calcium binding protein | Homo sapiens |
| 617 | AAH12464 | 755 | 100 | BC012464 prefoldin 2 | Homo sapiens |
| 617 | AF117237_1 | 755 | 100 | AF117237 prefoldin subunit 2 | Homo sapiens |
| 617 | BC026839 | 729 | 96 | Similar to prefoldin 2 | Mus musculus |
| 618 | AAH08230 | 849 | 100 | BC008230 ribosomal protein L12 | Homo sapiens |
| 618 | BC018321 | 848 | 99 | ribosomal protein L12 | Mus musculus |
| 618 | L06505 | 849 | 100 | ribosomal protein L12 | Homo sapiens |
| 619 | AAH12606 | 1665 | 100 | BC012606 Similar to proteasome | Homo sapiens |
| | | | | (prosome, macropain) 26S subunit, | |
| | | | | non-ATPase, 7 (Mov34 homolog) | |
| 619 | D50063 | 1637 | 98 | proteasome subunit p40/Mov34 | Homo sapiens |
| | | | | protein | |
| 619 | M64640 | 1597 | 96 | 36 kD protein | Mus musculus |
| 620 | AAH118345 | 1485 | 100 | BC011835 Similar to ATPase, | Homo sapiens |
| | | | | Na+/K+ transporting, beta 3 | |
| | | | | polypeptide | |
| 620 | AF005896 | 1485 | 100 | Na K-ATPase beta-3 subunit | Homo sapiens |
| 620 | U51478 | 1485 | 100 | sodium/potassium-transporting | Homo sapiens |
| | | | | ATPase beta-3 subunit | |
| 621 | AAH13041 | 5526 | 100 | BC013041 Unknown (protein for | Homo sapiens |
| | | | | MGC: 4781) | |
| 621 | M58028 | 5526 | 100 | ubiquitin-activating enzyme E1 | Homo sapiens |
| 621 | X56976 | 5516 | 99 | ubiquitin activating enzyme E1 | Homo sapiens |
| 622 | AAH13162 | 491 | 100 | BC013162 Similar to heat shock | Homo sapiens |
| | | | | protein, 30 kDa | |
| 622 | AF085359 | 491 | 100 | HSPC030 | Homo sapiens |
| 622 | AF537132 | 491 | 100 | selenoprotein K | Homo sapiens |
| 623 | AAH13175 | 992 | 100 | BC013175 Similar to novel RGD- | Homo sapiens |
| | | | | containing protein | |
| 623 | BC029249 | 958 | 96 | dynactin 6 | Mus musculus |
| 623 | D84145 | 992 | 100 | WS-3 | Homo sapiens |
| 624 | BC003825 | 2344 | 100 | tubulin, beta 5 | Mus musculus |
| 624 | X04663 | 2344 | 100 | beta-tubulin AA 1-444 (79 is 1st | Mus musculus |
| | | | | base in codon) | |
| 624 | X07011 | 2344 | 100 | c(beta)7 tubulin (AA 1-444) | Gallus |
| | | | | | gallus |
| 625 | A47413 | 3336 | 96 | CALPASTATINE HUMAINE | Homo sapiens |
| 625 | AAH13579 | 3420 | 100 | BC013579 Similar to calpastatin | Homo sapiens |
| 625 | D16217 | 3340 | 93 | calpastatin | Homo sapiens |
| 626 | BC024378 | 567 | 100 | defender against cell death 1 | Mus musculus |
| 626 | Y13335 | 567 | 100 | DAD-1 | Mus musculus |
| 626 | Y13336 | 567 | 100 | DAD-1 | Rattus |
| | | | | | norvegicus |
| 627 | AAH13975 | 3963 | 100 | BC013975 Similar to sema domain, | Homo sapiens |
| | | | | immunoglobulin domain (Ig), short | |
| | | | | basic domain, secreted, | |
| | | | | (semaphorin) 3B | |
| 627 | BC024220 | 3963 | 100 | Unknown (protein for MGC: 1483) | Homo sapiens |
| 627 | U28369 | 3951 | 99 | semaphorin V | Homo sapiens |
| 628 | AAH14142 | 3503 | 100 | BC014142 Similar to | Homo sapiens |
| | | | | glucuronidase, beta | |
| 628 | AX147652 | 3496 | 99 | unnamed protein product | Homo sapiens |
| 628 | M15182 | 3496 | 99 | beta-glucuronidase precursor (EC | Homo sapiens |
| | | | | 3.2.1.31) | |
| 629 | AB022163 | 2024 | 92 | mouse ortholog of the zebrafish | Mus musculus |
| | | | | hagoromo gene | |
| 629 | AF281859 | 2217 | 100 | dactylin | Homo sapiens |
| 629 | BC027031 | 2024 | 92 | f-box and WD-40 domain protein 4 | Mus musculus |
| 630 | AAH01377 | 1114 | 100 | BC001377 DKFZP586G1722 protein | Homo sapiens |
| 630 | AAH06514 | 1114 | 100 | BC006514 DKFZP586G1722 protein | Homo sapiens |
| 630 | AAH14391 | 1114 | 100 | BC014391 DKFZP586G1722 protein | Homo sapiens |
| 631 | AF159256_1 | 1147 | 100 | AF159256 Lps/Ran GTPase | Mus musculus |
| 631 | AF306457_1 | 1147 | 100 | AF306457 GTPase | Rattus |
| | | | | | norvegicus |
| 631 | BC014829 | 1147 | 100 | RAN, member RAS oncogene family | Mus musculus |
| 632 | AAH15000 | 1347 | 100 | BC015000 Similar to major | Homo sapiens |
| | | | | histocompatibility complex, class | |
| | | | | II, DP beta 1 | |
| 632 | M57466 | 1306 | 96 | light chain | Homo sapiens |
| 632 | X01426 | 1301 | 96 | SB-2-beta precursor polypeptide | Homo sapiens |
| | | | | (aa −29 to 229) | |
| 633 | AAH15039 | 1006 | 100 | BC015039 microfibrillar- | Homo sapiens |
| | | | | associated protein 2 | |
| 633 | AL049569 | 1006 | 100 | dJ37C10.4 (microfibrillar- | Homo sapiens |
| | | | | associated protein 2 | |
| | | | | (microfibril-associated | |
| | | | | glycoprotein precursor, MGAP1)) | |
| 633 | U19718 | 1006 | 100 | microfibril-associated | Homo sapiens |
| | | | | glycoprotein | |
| 634 | AF117383_1 | 746 | 100 | AF117383 placental protein 13; | Homo sapiens |
| | | | | PP13 | |
| 634 | AY055826 | 746 | 100 | placenta protein 13 | Homo sapiens |
| 634 | BC022257 | 510 | 69 | Similar to placental protein 13- | Homo sapiens |
| | | | | like protein | |
| 635 | AAH02387 | 2063 | 100 | BC002387 nucleosome assembly | Homo sapiens |
| | | | | protein 1-like 1 | |
| 635 | AAH15599 | 2063 | 100 | BC015599 nucleosome assembly | Homo sapiens |
| | | | | protein 1-like 1 | |
| 635 | AL162068 | 2063 | 100 | hypothetical protein | Homo sapiens |
| 636 | A03911 | 2017 | 99 | glia-derived neurite-promoting | Homo sapiens |
| | | | | factor (GdNPF) | |
| 636 | AAH15663 | 2040 | 100 | BC015663 Similar to serine (or | Homo sapiens |
| | | | | cysteine) proteinase inhibitor, | |
| | | | | clade E (nexin, plasminogen | |
| | | | | activator inhibitor type 1), | |
| | | | | member 2 | |
| 636 | M17783 | 2016 | 99 | glia-derived nexin precursor | Homo sapiens |
| 637 | AAH02403 | 3107 | 100 | BC002403 lectin, galactoside- | Homo sapiens |
| | | | | binding, soluble, 3 binding | |
| | | | | protein (galectin 6 binding | |
| | | | | protein) | |
| 637 | AAH02998 | 3107 | 100 | BC002998 lectin, galactoside- | Homo sapiens |
| | | | | binding, soluble, 3 binding | |
| | | | | protein (galectin 6 binding | |
| | | | | protein) | |
| 637 | AAH15761 | 3107 | 100 | BC015761 lectin, galactoside- | Homo sapiens |
| | | | | binding, soluble, 3 binding | |
| | | | | protein | |
| 638 | AAH15032 | 2122 | 100 | BC015032 ribosomal protein L3 | Homo sapiens |
| 638 | AAH15767 | 2122 | 100 | BC015767 ribosomal protein L3 | Homo sapiens |
| 638 | BC008003 | 2122 | 100 | ribosomal protein L3 | Homo sapiens |
| 639 | AF052694 | 2391 | 100 | alpha-tubulin | Meriones |
| | | | | | unguiculatus |
| 639 | BC002219 | 2391 | 100 | Similar to tubulin alpha 1 | Mus musculus |
| 639 | BC008117 | 2391 | 100 | tubulin alpha 1 | Mus musculus |
| 640 | AK054840 | 169 | 67 | unnamed protein product | Homo sapiens |
| 640 | AK097965 | 163 | 64 | unnamed protein product | Homo sapiens |
| 640 | AX400008 | 150 | 58 | unnamed protein product | Homo sapiens |
| 641 | AAH02449 | 1196 | 99 | BC002449 Similar to CG10641 gene | Homo sapiens |
| | | | | product | |
| 641 | AAH04128 | 1331 | 99 | BC004128 Unknown (protein for | Homo sapiens |
| | | | | IMAGE: 3940060) | |
| 641 | AK023674 | 1199 | 100 | unnamed protein product | Homo sapiens |
| 642 | AAH01537 | 2971 | 100 | BC001537 Unknown (protein for | Homo sapiens |
| | | | | MGC: 786) | |
| 642 | AB021743 | 2968 | 99 | PR65 | Mus musculus |
| 642 | BC006606 | 2968 | 99 | protein phosphatase 2 (formerly | Mus musculus |
| | | | | 2A), regulatory subunit A (PR | |
| | | | | 65), alpha isoform | |
| 643 | AAH09642 | 939 | 100 | BC009642 FXYD domain-containing | Homo sapiens |
| | | | | ion transport regulator 5 | |
| 643 | AB072911 | 939 | 100 | dysadherin | Homo sapiens |
| 643 | AF161462_1 | 923 | 98 | AF161462 HSPC113 | Homo sapiens |
| 644 | AAH09687 | 869 | 100 | BC009687 epithelial membrane | Homo sapiens |
| | | | | protein 2 | |
| 644 | AY057060 | 869 | 100 | epithelial membrane protein 2 | Homo sapiens |
| 644 | U52100 | 869 | 100 | XMP | Homo sapiens |
| 645 | AB012214 | 6727 | 78 | DNA cytosine 5 methyltransferase | Rattus |
| | | | | | Rattus |
| 645 | AF180682 | 6869 | 100 | DNA (cytosine-5)- | Homo sapiens |
| | | | | methyltransferase | |
| 645 | X63692 | 8587 | 100 | DNA (cytosine-5-)- | Homo sapiens |
| | | | | methyltransferase | |
| 646 | AAH15961 | 961 | 100 | BC015961 adrenomedullin | Homo sapiens |
| 646 | D14874 | 961 | 100 | adrenomedullin precursor | Homo sapiens |
| 646 | S73906 | 961 | 100 | adrenomedullin; AM | Homo sapiens |
| 647 | AAH15973 | 514 | 100 | BC015973 S100 calcium-binding | Homo sapiens |
| | | | | protein A10 (annexin II ligand, | |
| | | | | calpactin I, light polypeptide | |
| | | | | (p11)) | |
| 647 | M38591 | 514 | 100 | cellular ligand of annexin II | Homo sapiens |
| 647 | M81457 | 514 | 100 | calpactin I light chain | Homo sapiens |
| 648 | AAH16056 | 1137 | 100 | BC016056 claudin 3 | Homo sapiens |
| 648 | AB000714 | 1137 | 100 | RVP1 | Homo sapiens |
| 648 | AF007189 | 1137 | 100 | claudin 3 | Homo sapiens |
| 649 | AAH16142 | 1432 | 100 | BC016142 Similar to RIKEN cDNA | Homo sapiens |
| | | | | 2310032N20 gene | |
| 649 | AAH20773 | 1432 | 100 | BC020773 Unknown (protein for | Homo sapiens |
| | | | | MGC: 22685) | |
| 649 | AX405824 | 1432 | 100 | unnamed protein product | Homo sapiens |
| 650 | AF315378_1 | 1955 | 98 | AF315378 suppressor of | Rattus |
| | | | | profilin/p41 of actin-related | norvegicus |
| | | | | complex 2/3 | |
| 650 | BC039594 | 1964 | 99 | actin related protein 2/3 | Homo sapiens |
| | | | | complex, subunit 1A, 41 kDa | |
| 650 | Y08999 | 1980 | 100 | Sop2p-like protein | Homo sapiens |
| 651 | AF492677_1 | 383 | 100 | AF492677 odd homeobox 1 protein | Homo sapiens |
| | | | | isoform A | |
| 651 | AF492680_1 | 383 | 100 | AF492680 odd homeobox 1 protein | Homo sapiens |
| | | | | isoform A | |
| 651 | AF492681_1 | 383 | 100 | AF492681 odd homeobox 1 protein | Homo sapiens |
| | | | | isoform A | |
| 652 | AK056992 | 2163 | 100 | unnamed protein product | Homo sapiens |
| 652 | AX392121 | 2155 | 99 | unnamed protein product | Homo sapiens |
| 652 | AY033237 | 2155 | 99 | alcohol dehydrogenase 8 | Homo sapiens |
| 653 | AAH16748 | 485 | 100 | BC016748 ribosomal protein L37a | Homo sapiens |
| 653 | X14069 | 485 | 100 | ribosomal protein L37a (AA 1-92) | Rattus |
| | | | | | Rattus |
| 653 | X73331 | 485 | 100 | ribosomal protein L37a | Mus musculus |
| 654 | AAH12807 | 3702 | 100 | BC012807 Unknown (protein for | Homo sapiens |
| | | | | MGC: 3483) | |
| 654 | AAH14485 | 3702 | 100 | BC014485 Unknown (protein for | Homo sapiens |
| | | | | MGC: 23206) | |
| 654 | AAH16753 | 3702 | 100 | BC016753 Unknown (protein for | Homo sapiens |
| | | | | MGC: 1138) | |
| 655 | AAH15834 | 1724 | 100 | BC015834 annexin A2 | Homo sapiens |
| 655 | AAH16774 | 1724 | 100 | BC016774 annexin A2 | Homo sapiens |
| 655 | AAH21114 | 1724 | 100 | BC021114 annexin A2 | Homo sapiens |
| 656 | AAH17343 | 644 | 100 | BC017343 ribosomal protein L31 | Homo sapiens |
| 656 | BC008223 | 644 | 100 | ribosomal protein L31 | Mus musculus |
| 656 | X04809 | 644 | 100 | ribosomal protein L31 (AA 1-125) | Rattus |
| | | | | | norvegicus |
| 657 | AAH17378 | 2955 | 100 | BC017378 hexosaminidase B (beta | Homo sapiens |
| | | | | polypeptide) | |
| 657 | AF378118_1 | 2955 | 100 | AF378118 cervical cancer proto- | Homo sapiens |
| | | | | oncogene 7 | |
| 657 | M23294 | 2955 | 100 | beta-hexosaminidase beta-subunit | Homo sapiens |
| 658 | AAH12168 | 1373 | 100 | BC012168 Similar to proteasome | Homo sapiens |
| | | | | (prosome, macropain) subunit, | |
| | | | | beta type, 4 | |
| 658 | AAH17451 | 1373 | 100 | BC017451 proteasome (prosome, | Homo sapiens |
| | | | | macropain) subunit, beta type, 4 | |
| 658 | AAH17486 | 1373 | 100 | BC017486 proteasome (prosome, | Homo sapiens |
| | | | | macropain) subunit, beta type, 4 | |
| 659 | AAH06124 | 2618 | 100 | BC006124 IMP (inosine | Homo sapiens |
| | | | | monophosphate) dehydrogenase 2 | |
| 659 | AAH12840 | 2618 | 100 | BC012840 IMP (inosine | Homo sapiens |
| | | | | monophosphate) dehydrogenase 2 | |
| 659 | AAH15567 | 2618 | 100 | BC015567 IMP (inosine | Homo sapiens |
| | | | | monophosphate) dehydrogenase 2 | |
| 660 | AAH07457 | 1103 | 100 | BC007457 hypothetical protein | Homo sapiens |
| 660 | AAH20796 | 1103 | 100 | BC020796 hypothetical protein | Homo sapiens |
| | | | | HSPC177 | |
| 660 | AAH21168 | 1103 | 100 | BC021168 hypothetical protein | Homo sapiens |
| | | | | HSPC177 | |
| 661 | AAH04904 | 2894 | 99 | BC004904 nuclear RNA export | Homo sapiens |
| | | | | factor 1 | |
| 661 | BC028041 | 2894 | 99 | nuclear RNA export factor 1 | Homo sapiens |
| 661 | U80073 | 2905 | 100 | tip associating protein | Homo sapiens |
| 662 | AF064238_1 | 4503 | 97 | AF064238 smoothelin large isoform | Homo sapiens |
| | | | | L2 | |
| 662 | AY061971 | 4653 | 100 | smoothelin-B2 | Homo sapiens |
| 662 | AY061972 | 4533 | 96 | smoothelin-B3 | Homo sapiens |
| 663 | AAH17773 | 1214 | 100 | BC017773 triggering receptor | Homo sapiens |
| | | | | expressed on myeloid cells 1 | |
| 663 | AF196329_1 | 1214 | 100 | AF196329 triggering receptor | Homo sapiens |
| | | | | expressed on monocytes 1 | |
| 663 | AF287008_1 | 1214 | 100 | AF287008 triggering receptor | Homo sapiens |
| | | | | expressed on monocytes 1 | |
| 664 | AAH17891 | 579 | 100 | BC017891 vesicle-associated | Homo sapiens |
| | | | | membrane protein 5 (myobrevin) | |
| 664 | AF054825 | 579 | 100 | VAMP5 | Homo sapiens |
| 664 | AF077197 | 579 | 100 | VAMP5-like protein | Homo sapiens |
| 665 | AAH17931 | 819 | 100 | BC017931 Similar to RIKEN cDNA | Homo sapiens |
| | | | | 1110055A02 gene | |
| 665 | AF400652_1 | 819 | 100 | AF400652 thyroid hormone receptor | Homo sapiens |
| | | | | interacting protein 3 | |
| 665 | L40410 | 806 | 100 | thyroid receptor interactor | Homo sapiens |
| 666 | AAH18732 | 1606 | 100 | BC018732 Unknown (protein for | Homo sapiens |
| | | | | MGC: 31847) | |
| 666 | AF125533_1 | 1606 | 100 | AF125533 NADH-cytochrome b5 | Homo sapiens |
| | | | | reductase isoform | |
| 666 | AX083421 | 1606 | 100 | unnamed protein product | Homo sapiens |
| 667 | AAH02453 | 3263 | 100 | BC002453 heat shock 70 kD protein | Homo sapiens |
| | | | | 1A | |
| 667 | AAH09322 | 3263 | 100 | BC009322 heat shock 70 kD protein | Homo sapiens |
| | | | | 1A | |
| 667 | AAH18740 | 3263 | 100 | BC018740 heat shock 70 kD protein | Homo sapiens |
| | | | | 1A | |
| 668 | 1680052_1 | 2856 | 100 | S62076 lysosomal enzyme beta-N- | Homo sapiens |
| | | | | acetylhexosaminidase A | |
| 668 | AAH18927 | 2856 | 100 | BC018927 hexosaminidase A (alpha | Homo sapiens |
| | | | | polypeptide) | |
| 668 | M16424 | 2856 | 100 | beta-hexosaminidase alpha chain | Homo sapiens |
| 669 | AAH12992 | 2115 | 99 | BC012992 Similar to RNA binding | Homo sapiens |
| | | | | motif, single stranded | |
| | | | | interacting protein 1 | |
| 669 | AAH12993 | 2115 | 99 | BC012993 Similar to RNA binding | Homo sapiens |
| | | | | motif, single stranded | |
| | | | | interacting protein 1 | |
| 669 | AAH18951 | 2142 | 100 | BC018951 RNA binding motif, | Homo sapiens |
| | | | | single stranded interacting | |
| | | | | protein 1 | |
| 670 | AAH16346 | 908 | 100 | BC016346 Unknown (protein for | Homo sapiens |
| | | | | MGC: 24392) | |
| 670 | AAH16354 | 908 | 100 | BC016354 Unknown (protein for | Homo sapiens |
| | | | | MGC: 24480) | |
| 670 | AAH18990 | 908 | 100 | BC018990 Unknown (protein for | Homo sapiens |
| | | | | MGC: 20176) | |
| 671 | AAH01206 | 2235 | 99 | BC001206 Unknown (protein for | Homo sapiens |
| | | | | MGC: 3208) | |
| 671 | AK025822 | 3505 | 99 | unnamed protein product | Homo sapiens |
| 671 | AL833865 | 3414 | 100 | hypothetical protein | Homo sapiens |
| 672 | AAH06772 | 769 | 100 | BC006772 ribosomal protein S13 | Homo sapiens |
| 672 | BC029732 | 769 | 100 | ribosomal protein S13 | Homo sapiens |
| 672 | X53378 | 769 | 100 | ribosomal protein S13 | Rattus |
| | | | | | Rattus |
| 673 | J05192 | 1982 | 100 | alpha-actin | Homo sapiens |
| 673 | X06801 | 1968 | 99 | alpha-actin (AA 1-377) | Rattus |
| | | | | | Rattus |
| 673 | X13297 | 1968 | 99 | alpha-actin (AA 1-377) | Mus musculus |
| 674 | AAH00260 | 1675 | 99 | BC000260 aldo-keto reductase | Homo sapiens |
| | | | | family 1, member B1 (aldose | |
| | | | | reductase) | |
| 674 | AAH10391 | 1675 | 99 | BC010391 aldo-keto reductase | Homo sapiens |
| | | | | family 1, member B1 (aldose | |
| | | | | reductase) | |
| 674 | J05017 | 1677 | 100 | aldose reductase | Homo sapiens |
| 675 | M22246 | 2623 | 100 | aromatase precursor (EC | Homo sapiens |
| | | | | 1.14.14.1) | |
| 675 | X13589 | 2623 | 100 | aromatase (AA 1-503) | Homo sapiens |
| 675 | Y07508 | 2620 | 99 | aromatase (AA 1-503) | Homo sapiens |
| 676 | M59916 | 3466 | 100 | acid sphingomyelinase | Homo sapiens |
| 676 | M81780 | 3450 | 99 | acid sphingomyelinase | Homo sapiens |
| 676 | X63600 | 3461 | 99 | acid sphingomyelinase | Homo sapiens |
| 677 | AAH21993 | 1707 | 100 | BC021993 guanine nucleotide | Homo sapiens |
| | | | | binding protein (G protein), beta | |
| | | | | polypeptide 2-like 1 | |
| 677 | M24193 | 1707 | 100 | MHC B complex protein 12.3 | Gallus |
| | | | | | gallus |
| 677 | X75313 | 1707 | 100 | B complex protein mRNA 12-3 | Mus musculus |
| 678 | AAH05358 | 642 | 100 | BC005358 non-histone chromosome | Homo sapiens |
| | | | | protein 2 (S. cerevisiae)-like 1 | |
| 678 | AAH19282 | 642 | 100 | BC019282 non-histone chromosome | Homo sapiens |
| | | | | protein 2 (S. cerevisiae)-like 1 | |
| 678 | BC026755 | 642 | 100 | Similar to sperm specific antigen 1 | Mus musculus |
| 679 | AAH19296 | 2747 | 100 | BC019296 chaperonin containing | Homo sapiens |
| | | | | TCP1, subunit 7 (eta) | |
| 679 | AF026292 | 2747 | 100 | chaperonin containing t-complex | Homo sapiens |
| | | | | polypeptide 1, eta subunit; CCT- | |
| | | | | eta | |
| 679 | BC008255 | 2628 | 95 | chaperonin subunit 7 (eta) | Mus musculus |
| 680 | BC035220 | 3879 | 99 | similar to complement component | Homo sapiens |
| 680 | M14058 | 3885 | 100 | human complement C1r | Homo sapiens |
| 680 | X04701 | 3879 | 99 | precursor of C1r (AA −17 to 688) | Homo sapiens |
| 681 | J04080 | 3789 | 100 | complement component C1s | Homo sapiens |
| 681 | M18767 | 3789 | 100 | complement subcomponent C1s | Homo sapiens |
| | | | | precursor | |
| 681 | X06596 | 3789 | 100 | complement protein C1s precursor | Homo sapiens |
| 682 | AAH03175 | 2072 | 100 | BC003175 N-myc downstream | Homo sapiens |
| | | | | regulated | |
| 682 | AF004162 | 2072 | 100 | nickel-specific induction protein | Homo sapiens |
| 682 | D87953 | 2072 | 100 | RTP | Homo sapiens |
| 683 | AF085250 | 759 | 100 | calmodulin | Perca |
| | | | | | flavescens |
| 683 | D83350 | 759 | 100 | calmodulin | Anas |
| | | | | | platyrhynchos |
| 683 | K01945 | 759 | 100 | calmodulin (cDNA clone 71) | Xenopus |
| | | | | | laevis |
| 684 | AAH07911 | 2282 | 100 | BC007911 calreticulin | Homo sapiens |
| 684 | AAH20493 | 2282 | 100 | BC020493 calreticulin | Homo sapiens |
| 684 | AY047586 | 2282 | 100 | calreticulin | Homo sapiens |
| 685 | AAH16300 | 533 | 100 | BC016300 S100 calcium-binding | Homo sapiens |
| | | | | protein A4 (calcium protein, | |
| | | | | calvasculin, metastasin, murine | |
| | | | | placental homolog) | |
| 685 | Z18950 | 533 | 100 | CAPL | Homo sapiens |
| 685 | Z33457 | 533 | 100 | mts1 | Homo sapiens |
| 686 | AAH01506 | 710 | 100 | BC001506 CD59 antigen p18-20 | Homo sapiens |
| | | | | (antigen identified by monoclonal | |
| | | | | antibodies 16.3A5, EJ16, EJ30, | |
| | | | | EL32 and G344) | |
| 686 | X16447 | 710 | 100 | precursor polypeptide (AA −25 to | Homo sapiens |
| | | | | 103) | |
| 686 | X17198 | 710 | 100 | CD59 antigen precursor | Homo sapiens |
| 687 | AAH06290 | 898 | 100 | BC006290 chorionic gonadotropin, | Homo sapiens |
| | | | | beta polypeptide | |
| 687 | BC022796 | 898 | 100 | Unknown (protein for MGC: 39080) | Homo sapiens |
| 687 | BC030994 | 898 | 100 | chorionic gonadotropin, beta | Homo sapiens |
| | | | | polypeptide | |
| 688 | J03225 | 1674 | 100 | lipoprotein-associated | Homo sapiens |
| | | | | coagulation inhibitor precursor | |
| 688 | M58650 | 1674 | 100 | lipoprotein associated | Homo sapiens |
| | | | | coagulation inhibitor | |
| 688 | M59499 | 1674 | 100 | lipoprotein-associated | Homo sapiens |
| | | | | coagulation inhibitor | |
| 689 | AAH14167 | 1072 | 100 | BC014167 Unknown (protein for | Homo sapiens |
| | | | | MGC: 20892) | |
| 689 | AAH20804 | 1072 | 100 | BC020804 ribosomal protein L13 | Homo sapiens |
| 689 | BC027463 | 1072 | 100 | ribosomal protein L13 | Homo sapiens |
| 690 | AAH21136 | 1333 | 100 | BC021136 Unknown (protein for | Homo sapiens |
| | | | | MGC: 32056) | |
| 690 | AF097362_1 | 1333 | 100 | AF097362 gamma-interferon | Homo sapiens |
| | | | | inducible lysosomal thiol | |
| | | | | reductase | |
| 690 | BC031020 | 1329 | 99 | interferon, gamma-inducible | Homo sapiens |
| | | | | protein 30 | |
| 691 | AAH07008 | 929 | 100 | BC007008 crystallin, alpha B | Homo sapiens |
| 691 | M28638 | 929 | 100 | alpha-B2-crystallin | Homo sapiens |
| 691 | S45630 | 929 | 100 | alpha B-crystallin | Homo sapiens |
| 692 | BC003775 | 1169 | 99 | casein kinase II, beta subunit | Mus musculus |
| 692 | M30448 | 1174 | 100 | casein kinase II beta subunit | Homo sapiens |
| 692 | M59458 | 1169 | 99 | casein kinase II beta subunit | Gallus |
| | | | | | gallus |
| 693 | M77820 | 6209 | 50 | fibronectin | Xenopus |
| | | | | | laevis |
| 693 | X02761 | 12570 | 99 | fibronectin precursor | Homo sapiens |
| 693 | X15906 | 7305 | 65 | precursor polypeptide (AA −32 to | Rattus |
| | | | | 2445) | norvegicus |
| 694 | BC032329 | 2740 | 99 | cytochrome P450, subfamily XIA | Homo sapiens |
| | | | | (cholesterol side chain cleavage) | |
| 694 | M14565 | 2753 | 100 | cholesterol side-chain cleavage | Homo sapiens |
| | | | | enzyme P450scc (EC 1.14.15.67) | |
| 694 | X05367 | 2734 | 99 | desmolase | Homo sapiens |
| 695 | AF318372_1 | 592 | 99 | AF318372 unknown | Homo sapiens |
| 695 | AJ318881 | 601 | 100 | XAGE-3 protein | Homo sapiens |
| 695 | AJ318893 | 601 | 100 | XAGE-3 protein | Homo sapiens |
| 696 | AAH18641 | 2406 | 100 | BC018641 eukaryotic translation | Homo sapiens |
| | | | | elongation factor 1 alpha 1 | |
| 696 | AAH21686 | 2406 | 100 | BC021686 eukaryotic translation | Homo sapiens |
| | | | | elongation factor 1 alpha 1 | |
| 696 | BC028674 | 2406 | 100 | eukaryotic translation elongation | Homo sapiens |
| | | | | factor 1 alpha 1 | |
| 697 | AAH07044 | 2377 | 100 | BC007044 fibrinogen, gamma | Homo sapiens |
| | | | | polypeptide | |
| 697 | AAH21674 | 2377 | 100 | BC021674 fibrinogen, gamma | Homo sapiens |
| | | | | polypeptide | |
| 697 | AF350254_1 | 2377 | 100 | AF350254 fibrinogen gamma chain, | Homo sapiens |
| | | | | isoform gamma-A precursor | |
| 698 | AAH07785 | 2357 | 100 | BC007785 Unknown (protein for | Homo sapiens |
| | | | | IMAGE: 2819608) | |
| 698 | AAH21565 | 2387 | 100 | BC021565 Unknown (protein for | Homo sapiens |
| | | | | MGC: 31904) | |
| 698 | AF121858_1 | 2387 | 100 | AF121858 sorting nexin 8 | Homo sapiens |
| 699 | AAH05929 | 1222 | 100 | BC005929 proteoglycan 2, bone | Homo sapiens |
| | | | | marrow (natural killer cell | |
| | | | | activator, eosinophil granule | |
| | | | | major basic protein) | |
| 699 | X14088 | 1222 | 100 | major basic protein precusor (AA | Homo sapiens |
| | | | | 1-222) | |
| 699 | Y00809 | 1222 | 100 | major basic preproprotein (AA −15 | Homo sapiens |
| | | | | to 207) | |
| 700 | AL050169 | 951 | 100 | hypothetical protein | Homo sapiens |
| 700 | M34046 | 951 | 100 | placental protein 14 precursor | Homo sapiens |
| | | | | (PP14) | |
| 700 | M61886 | 951 | 100 | pregnancy-associated endometrial | Homo sapiens |
| | | | | alpha2-globulin | |
| 701 | AAH15641 | 2224 | 100 | BC015641 enolase 1, (alpha) | Homo sapiens |
| 701 | BC022545 | 2224 | 100 | enolase 1, (alpha) | Homo sapiens |
| 701 | BC027725 | 2224 | 100 | enolase 1, (alpha) | Homo sapiens |
| 702 | AF272142 | 2649 | 100 | cytochrome P450 | Homo sapiens |
| 702 | BC032594 | 2649 | 100 | cytochrome P450, subfamily IIJ | Homo sapiens |
| | | | | (arachidonic acid epoxygenase) | |
| | | | | polypeptide 2 | |
| 702 | U37143 | 2649 | 100 | cytochrome P450 monooxygenase | Homo sapiens |
| | | | | CYP2J2 | |
| 703 | AAH17123 | 1367 | 100 | BC017123 ribosomal protein S3A | Homo sapiens |
| 703 | AAH19072 | 1367 | 100 | BC019072 ribosomal protein S3A | Homo sapiens |
| 703 | BC030161 | 1367 | 100 | ribosomal protein S3A | Homo sapiens |
| 704 | AB042200 | 2004 | 88 | low-density lipoprotein receptor- | Mus musculus |
| | | | | related protein 9 | |
| 704 | AL834518 | 2154 | 99 | hypothetical protein | Homo sapiens |
| 704 | AX359701 | 2268 | 99 | unnamed protein product | Homo sapiens |
| 705 | BC040431 | 5104 | 99 | glucosidase, alpha; acid (Pompe | Homo sapiens |
| | | | | disease, glycogen storage disease | |
| | | | | type II) | |
| 705 | M34424 | 5105 | 100 | acid alpha-glucosidase | Homo sapiens |
| 705 | X55080 | 5105 | 100 | acid alpha-glucosidase | Homo sapiens |
| 706 | AAH10914 | 768 | 100 | BC010914 Similar to hemoglobin, | Homo sapiens |
| | | | | gamma G | |
| 706 | BC029387 | 768 | 100 | hemoglobin, gamma G | Homo sapiens |
| 706 | M91037 | 768 | 100 | G-gamma globin | Homo sapiens |
| 707 | AY148100 | 4568 | 100 | colony stimulating factor 3 | Homo sapiens |
| | | | | receptor (granulocyte) | |
| 707 | M59818 | 4568 | 100 | granulocyte colony-stimulating | Homo sapiens |
| | | | | factor receptor | |
| 707 | X55721 | 4568 | 100 | granulocyte colony stimulating | Homo sapiens |
| | | | | factor receptor 25-1 | |
| 708 | AAH00013 | 1579 | 100 | BC000013 insulin-like growth | Homo sapiens |
| | | | | factor binding protein 3 | |
| 708 | AAH18962 | 1579 | 100 | BC018962 insulin-like growth | Homo sapiens |
| | | | | factor binding protein 3 | |
| 708 | M35878 | 1579 | 100 | growth factor-binding protein-3 | Homo sapiens |
| | | | | precursor | |
| 709 | M23595 | 1413 | 100 | insulin-like growth factor | Homo sapiens |
| | | | | binding protein 1 | |
| 709 | M59316 | 1413 | 100 | insulin-like growth factor | Homo sapiens |
| | | | | binding protein-1 | |
| 709 | X12385 | 1413 | 100 | PP12 precorsor (AA −25 to 234) | Homo sapiens |
| 710 | BC041218 | 676 | 100 | H3 histone, family 3B | Xenopus |
| | | | | | laevis |
| 710 | M11393 | 676 | 100 | histone 3.3 | Gallus |
| | | | | | gallus |
| 710 | Y00392 | 676 | 100 | histone H3.3B (AA 1-136) | Gallus |
| | | | | | gallus |
| 711 | AF523281 | 1916 | 99 | MHC class Ib antigen | Homo sapiens |
| 711 | AF523283 | 1916 | 99 | MHC class Ib antigen | Homo sapiens |
| 711 | M20022 | 1922 | 100 | HLA-E class I protein precursor | Homo sapiens |
| 712 | AAH03569 | 1980 | 100 | BC003569 E74-like factor 3 (ets | Homo sapiens |
| | | | | domain transcription factor, | |
| | | | | epithelial-specific) | |
| 712 | AF017307 | 1980 | 100 | Ets-related transcription factor | Homo sapiens |
| 712 | AF110184_1 | 1980 | 100 | AF110184 epithelium-restricted | Homo sapiens |
| | | | | Ets protein ESX | |
| 713 | AF523305 | 1810 | 100 | MHC class Ib antigen | Homo sapiens |
| 713 | AF523306 | 1810 | 100 | MHC class Ib antigen | Homo sapiens |
| 713 | AF523309 | 1810 | 100 | MHC class Ib antigen | Homo sapiens |
| 714 | AAH09507 | 836 | 99 | BC009507 Unknown (protein for | Homo sapiens |
| | | | | MGC: 3945) | |
| 714 | AY168648 | 836 | 99 | ubiquitin-like protein ISG15 | Homo sapiens |
| 714 | M13755 | 839 | 100 | 17-kDa protein | Homo sapiens |
| 715 | AB033054 | 2549 | 95 | KIAA1228 protein | Homo sapiens |
| 715 | AF099138 | 1351 | 51 | GLUT4 vesicle protein | Rattus |
| | | | | | norvegicus |
| 715 | BC011482 | 1358 | 50 | Similar to membrane bound C2 | Mus musculus |
| | | | | domain containing protein | |
| 716 | AAH10853 | 1129 | 100 | BC010853 Similar to integral type | Homo sapiens |
| | | | | I protein | |
| 716 | AAH17495 | 1129 | 100 | BC017495 integral type I protein | Homo sapiens |
| 716 | BC022232 | 1129 | 100 | integral type I protein | Homo sapiens |
| 717 | AF357203_1 | 1832 | 97 | AF357203 MAP kinase phosphatase-1 | Rattus |
| | | | | | norvegicus |
| 717 | BC022463 | 1886 | 100 | dual specificity phosphatase 1 | Homo sapiens |
| 717 | X68277 | 1886 | 100 | protein-tyrosine phosphatase | Homo sapiens |
| 718 | AAH09407 | 687 | 100 | BC009407 ribosomal protein S17 | Homo sapiens |
| 718 | AAH19899 | 687 | 100 | BC019899 ribosomal protein S17 | Homo sapiens |
| 718 | BC022370 | 687 | 100 | ribosomal protein S17 | Homo sapiens |
| 719 | AAH15105 | 1370 | 100 | BC015105 proteasome (prosome, | Homo sapiens |
| | | | | macropain) subunit, alpha type, 1 | |
| 719 | AAH15356 | 1370 | 100 | BC015356 proteasome (prosome, | Homo sapiens |
| | | | | macropain) subunit, alpha type, 1 | |
| 719 | BC022372 | 1370 | 100 | proteasome (prosome, macropain) | Homo sapiens |
| | | | | subunit, alpha type, 1 | |
| 720 | AAH03352 | 723 | 100 | BC003352 tumor protein, | Homo sapiens |
| | | | | translationally-controlled 1 | |
| 720 | BC022436 | 723 | 100 | Similar to tumor protein, | Homo sapiens |
| | | | | translationally-controlled 1 | |
| 720 | X16064 | 723 | 100 | tumor protein (AA 1-172) | Homo sapiens |
| 721 | BC023006 | 3271 | 100 | Unknown (protein for MGC: 30059) | Homo sapiens |
| 721 | M27024 | 3271 | 100 | heat shock protein | Homo sapiens |
| 721 | X15183 | 3271 | 100 | 90 kDa heat-shock protein (AA 1-732) | Homo sapiens |
| 722 | AAH08064 | 2806 | 100 | BC008064 monoamine oxidase A | Homo sapiens |
| 722 | M68857 | 2806 | 100 | monoamine oxidase A | Homo sapiens |
| 722 | M69226 | 2806 | 100 | monoamine oxidase A | Homo sapiens |
| 723 | M84378 | 1952 | 100 | lymphocyte antigen | Homo sapiens |
| 723 | M84379 | 1945 | 99 | MHC class I lymphocyte antigen | Homo sapiens |
| 723 | Z27120 | 1946 | 99 | HLA-A2 SLU | Homo sapiens |
| 724 | BC032350 | 1338 | 100 | major histocompatibility complex, | Homo sapiens |
| | | | | class II, DR alpha | |
| 724 | M60334 | 1338 | 100 | cell surface glycoprotein | Homo sapiens |
| 724 | Z84814 | 1338 | 100 | dJ172K2.4.1 (major | Homo sapiens |
| | | | | histocompatibility complex, class | |
| | | | | II, DR alpha, isoform 1) | |
| 725 | AAH02476 | 803 | 100 | BC002476 non-metastatic cells 2, | Homo sapiens |
| | | | | protein (NM23B) expressed in | |
| 725 | L16785 | 803 | 100 | c-myc transcription factor | Homo sapiens |
| 725 | X58965 | 803 | 100 | NM23-H2 protein | Homo sapiens |
| 726 | AAH11645 | 1585 | 100 | BC011645 Similar to Ras-related | Homo sapiens |
| | | | | associated with diabetes | |
| 726 | L24564 | 1585 | 100 | Rad | Homo sapiens |
| 726 | U46165 | 1565 | 99 | Rad GTPase | Homo sapiens |
| 727 | AAH03358 | 1151 | 100 | BC003358 ribosomal protein L10 | Homo sapiens |
| 727 | BC026276 | 1151 | 100 | ribosomal protein L10 | Homo sapiens |
| 727 | L44140 | 1151 | 100 | QM | Homo sapiens |
| 728 | A84527 | 2912 | 100 | unnamed protein product | unidentified |
| 728 | AX024042 | 2912 | 100 | fusion between Aequorea victoria | synthetic |
| | | | | and human | construct |
| 728 | M62399 | 2912 | 100 | NF-kappa-B transcription factor | Homo sapiens |
| 729 | AAH10860 | 2071 | 100 | BC010860 serine (or cysteine) | Homo sapiens |
| | | | | proteinase inhibitor, clade E | |
| | | | | (nexin, plasminogen activator | |
| | | | | inhibitor type 1), member 1 | |
| 729 | AF386492_1 | 2071 | 100 | AF386492 serine-cysteine | Homo sapiens |
| | | | | proteinase inhibitor clade E | |
| | | | | member 1 | |
| 729 | X04429 | 2071 | 100 | precursor polypeptide | Homo sapiens |
| 730 | AAH00491 | 1316 | 100 | BC000491 proliferating cell | Homo sapiens |
| | | | | nuclear antigen | |
| 730 | AF347680_1 | 1316 | 100 | AF347680 proliferating cell | Macaca |
| | | | | nuclear antigen | fascicularis |
| 730 | AL121924 | 1316 | 100 | dJ746J20.2 (proliferating cell | Homo sapiens |
| | | | | nuclear antigen) | |
| 731 | AC007165 | 2130 | 100 | interleukin-1 receptor type II | Homo sapiens |
| 731 | AY124010 | 2130 | 100 | interleukin 1 receptor, type II | Homo sapiens |
| 731 | X59770 | 2130 | 100 | type II interleukin-1 receptor | Homo sapiens |
| 732 | BC041071 | 2366 | 100 | protein phosphatase 2 (formerly | Homo sapiens |
| | | | | 2A), regulatory subunit B (PR | |
| | | | | 52), alpha isoform | |
| 732 | M64929 | 2366 | 100 | protein phosphatase-2A subunit- | Homo sapiens |
| | | | | alpha | |
| 732 | U09356 | 2366 | 100 | protein phosphatase 2A1 B alpha | Oryctolagus |
| | | | | subunit | cuniculus |
| 733 | M34421 | 2279 | 99 | pregnancy-specific beta-1 | Homo sapiens |
| | | | | glycoprotein precursor | |
| 733 | M34481 | 2279 | 99 | pregnancy-specific beta-1- | Homo sapiens |
| | | | | glycoprotein 11 | |
| 733 | M58591 | 2291 | 100 | PSG11 | Homo sapiens |
| 734 | BC018254 | 703 | 97 | retinol binding protein 1, | Mus musculus |
| | | | | cellular | |
| 734 | M11433 | 724 | 100 | retinol-binding protein | Homo sapiens |
| 734 | X07437 | 724 | 100 | cellular retinol binding protein | Homo sapiens |
| 735 | J03149 | 4237 | 83 | M-CSF receptor | Felis catus |
| 735 | U63963 | 5158 | 100 | CSF-1 receptor | Homo sapiens |
| 735 | X03663 | 5150 | 99 | put. c-fms precursor | Homo sapiens |
| 736 | BC005580 | 902 | 100 | polymerase (RNA) II (DNA | Mus musculus |
| | | | | directed) polypeptide G | |
| 736 | U52427 | 902 | 100 | RNA polymerase II seventh subunit | Homo sapiens |
| 736 | Z71925 | 902 | 100 | RNA polymerase II RPB7 subunit- | Rattus |
| | | | | like protein | norvegicus |
| 737 | AAH11601 | 658 | 100 | BC011601 interferon, alpha- | Homo sapiens |
| | | | | inducible protein (clone IFI-6- | |
| | | | | 16) | |
| 737 | AAH15603 | 658 | 100 | BC015603 interferon, alpha- | Homo sapiens |
| | | | | inducible protein (clone IFI-6- | |
| | | | | 16) | |
| 737 | D11384 | 652 | 98 | ORF | Pan |
| | | | | | troglodytes |
| 738 | AF147083 | 1683 | 100 | gamma-glutamyl hydrolase | Homo sapiens |
| 738 | BC025025 | 1683 | 100 | gamma-glutamyl hydrolase | Homo sapiens |
| | | | | (conjugase, | |
| | | | | folylpolygammaglutamyl hydrolase) | |
| 738 | U55206 | 1683 | 100 | human gamma-glutamyl hydrolase | Homo sapiens |
| 739 | AAH10418 | 1552 | 100 | BC010418 laminin receptor 1 | Homo sapiens |
| | | | | (67 kD, ribosomal protein SA) | |
| 739 | AAH13827 | 1552 | 100 | BC013827 laminin receptor 1 | Homo sapiens |
| | | | | (67 kD, ribosomal protein SA) | |
| 739 | U43901 | 1552 | 100 | 37 kD laminin receptor | Homo sapiens |
| | | | | precursor/p40 ribosome associated | |
| | | | | protein | |
| 740 | BC026033 | 4101 | 100 | gelsolin (amyloidosis, Finnish | Homo sapiens |
| | | | | type) | |
| 740 | X04412 | 4101 | 100 | plasma gelsolin | Homo sapiens |
| 740 | X13871 | 3849 | 94 | gelsolin | Sus scrofa |
| 741 | AF304463_1 | 2813 | 100 | AF304463 calcium transport | Homo sapiens |
| | | | | protein CaT1 | |
| 741 | AF365927_1 | 2813 | 100 | AF365927 calcium transport | Homo sapiens |
| | | | | protein CaT1 | |
| 741 | AF365928_1 | 2946 | 100 | AF365928 calcium transport | Homo sapiens |
| | | | | protein CaT1 | |
| 742 | AY040542 | 2353 | 100 | sialic acid binding | Homo sapiens |
| | | | | immunoglobulin-like lectin 6 | |
| 742 | D86358 | 2353 | 100 | CD33L1 | Homo sapiens |
| 742 | U71382 | 2246 | 97 | OB binding protein-1 | Homo sapiens |
| 743 | AF286534_1 | 1115 | 100 | AF286534 GTP-binding protein | Rattus |
| | | | | RAB11B | norvegicus |
| 743 | AF498947_1 | 1115 | 100 | AF498947 small GTP binding | Homo sapiens |
| | | | | protein RAB11B | |
| 743 | X79780 | 1110 | 99 | YPT3 | Homo sapiens |
| 744 | BC010604 | 1253 | 99 | ribosomal protein S6 | Mus musculus |
| 744 | BC027620 | 1256 | 100 | ribosomal protein S6 | Homo sapiens |
| 744 | Z54209 | 1253 | 99 | rpS6 | Mus musculus |
| 745 | AJ005654 | 703 | 100 | secondary lymphoid tissue | Homo sapiens |
| | | | | chemokine | |
| 745 | BC027918 | 703 | 100 | small inducible cytokine | Homo sapiens |
| | | | | subfamily A (Cys—Cys), member 21 | |
| 745 | U88320 | 703 | 100 | beta chemokine Exodus-2 | Homo sapiens |
| 746 | AF148856_2 | 6613 | 98 | AF148856 unknown | Homo sapiens |
| 746 | AF149422_2 | 6615 | 98 | AF149422 unknown | Homo sapiens |
| 746 | U93564 | 6714 | 100 | putative p150 | Homo sapiens |
| 747 | AK012013 | 1608 | 100 | unnamed protein product | Mus musculus |
| 747 | BC003742 | 1608 | 100 | 26S proteasome-associated pad1 | Mus musculus |
| | | | | homolog | |
| 747 | U86782 | 1608 | 100 | 26S proteasome-associated pad1 | Homo sapiens |
| | | | | homolog | |
| 748 | AK011431 | 484 | 100 | unnamed protein product | Mus musculus |
| 748 | BC041218 | 676 | 100 | H3 histone, family 3B | Xenopus |
| | | | | | laevis |
| 748 | Y00392 | 676 | 100 | histone H3.3B (AA 1-136) | Gallus |
| | | | | | gallus |
| 749 | AAH11262 | 2716 | 99 | BC011262 Unknown (protein for | Homo sapiens |
| | | | | MGC: 18226) | |
| 749 | AF006043 | 2719 | 100 | 3-phosphoglycerate dehydrogenase | Homo sapiens |
| 749 | AF171237_1 | 2719 | 100 | AF171237 3-phosphoglycerate | Homo sapiens |
| | | | | dehydrogenase | |
| 750 | AF109197_1 | 1254 | 100 | AF109197 nuclear chloride channel | Homo sapiens |
| 750 | AJ012008 | 1251 | 99 | RNCC protein | Homo sapiens |
| 750 | U93205 | 1254 | 100 | nuclear chloride ion channel | Homo sapiens |
| | | | | protein | |
| 751 | BC029402 | 1311 | 100 | proteasome (prosome, macropain) | Homo sapiens |
| | | | | subunit, alpha type, 3 | |
| 751 | BC038990 | 1307 | 99 | Similar to proteasome (prosome, | Homo sapiens |
| | | | | macropain) subunit, alpha type, 3 | |
| 751 | D00762 | 1307 | 99 | proteasome subunit C8 | Homo sapiens |
| 752 | AB062400 | 671 | 100 | Similar to ribosomal protein S15a | Homo sapiens |
| 752 | BC030569 | 671 | 100 | ribosomal protein S15a | Homo sapiens |
| 752 | X77953 | 671 | 100 | ribosomal protein S15a | Rattus |
| | | | | | norvegicus |
| 753 | AB062438 | 1926 | 100 | OK/SW-CL.30 | Homo sapiens |
| 753 | BC025832 | 1581 | 90 | Unknown (protein for MGC: 37936) | Mus musculus |
| 753 | BC036872 | 1912 | 99 | Unknown (protein for | Homo sapiens |
| | | | | IMAGE: 5225889) | |
| 754 | AAH16706 | 2319 | 100 | BC016706 eukaryotic translation | Homo sapiens |
| | | | | initiation factor 3, subunit 6 | |
| | | | | (48 kD) | |
| 754 | AAH21679 | 2319 | 100 | BC021679 eukaryotic translation | Homo sapiens |
| | | | | initiation factor 3, subunit 6 | |
| | | | | (48 kD) | |
| 754 | BC029177 | 2319 | 100 | eukaryotic translation initiation | Mus musculus |
| | | | | factor 3, subunit 6 (48 kD) | |
| 755 | AAH04138 | 1465 | 99 | BC004138 ribosomal protein L6 | Homo sapiens |
| 755 | BC031009 | 1475 | 100 | ribosomal protein L6 | Homo sapiens |
| 755 | BC032299 | 1465 | 99 | ribosomal protein L6 | Homo sapiens |
| 756 | AAH15813 | 2331 | 100 | BC015813 eukaryotic translation | Homo sapiens |
| | | | | elongation factor 1 gamma | |
| 756 | BC028179 | 2331 | 100 | eukaryotic translation elongation | Homo sapiens |
| | | | | factor 1 gamma | |
| 756 | BC031012 | 2331 | 100 | eukaryotic translation elongation | Homo sapiens |
| | | | | factor 1 gamma | |
| 757 | AAH04927 | 1051 | 100 | BC004927 Unknown (protein for | Homo sapiens |
| | | | | MGC: 10432) | |
| 757 | AF013711 | 1051 | 100 | 22 kDa actin-binding protein | Homo sapiens |
| 757 | D84342 | 1051 | 100 | SM22 alpha | Homo sapiens |
| 758 | AB007510 | 12543 | 100 | PRP8 protein | Homo sapiens |
| 758 | AB047391 | 12461 | 99 | pre-mRNA processing 8 protein | Mus musculus |
| 758 | AF092565 | 12477 | 99 | splicing factor Prp8 | Homo sapiens |
| 759 | AAH10034 | 3127 | 100 | BC010034 thyroid autoantigen 70 kD | Homo sapiens |
| | | | | (Ku antigen) | |
| 759 | AAH12154 | 3127 | 100 | BC012154 Similar to thyroid | Homo sapiens |
| | | | | autoantigen 70 kD (Ku antigen) | |
| 759 | AAH18259 | 3127 | 100 | BC018259 thyroid autoantigen 70 kD | Homo sapiens |
| | | | | (Ku antigen) | |
| 760 | AL050369 | 2492 | 99 | hypothetical protein | Homo sapiens |
| 760 | AY040822 | 2500 | 100 | U4/U6 snRNP-associated 61 kDa | Homo sapiens |
| | | | | protein | |
| 760 | BC018376 | 2479 | 98 | Unknown (protein for MGC: 19247) | Mus musculus |
| 761 | AF343440_1 | 454 | 98 | AF343440 lysosomal H+ | Canis |
| | | | | transporting-ATPase subunit M9.2 | familiaris |
| 761 | AX400012 | 459 | 100 | unnamed protein product | Homo sapiens |
| 761 | Y15286 | 459 | 100 | vacuolar proton-ATPase subunit | Homo sapiens |
| | | | | M9.2 | |
| 762 | AAH01273 | 1831 | 99 | BC001273 galactose-4-epimerase, | Homo sapiens |
| | | | | UDP- | |
| 762 | AF022382 | 1835 | 100 | UDP-galactose 4′ epimerase | Homo sapiens |
| 762 | L41668 | 1835 | 100 | UDP-galactose-4-epimerase | Homo sapiens |
| 763 | AAH05270 | 900 | 99 | BC005270 NADH dehydrogenase | Homo sapiens |
| | | | | (ubiquinone) Fe-S protein 4 | |
| | | | | (18 kD) (NADH-coenzyme Q | |
| | | | | reductase) | |
| 763 | AF020351 | 904 | 100 | NADH: ubiquinone oxidoreductase 18 kDa | Homo sapiens |
| | | | | IP subunit | |
| 763 | X63215 | 839 | 90 | NADH dehydrogenase | Bos taurus |
| 764 | AAH04963 | 674 | 100 | BC004963 ATP synthase, H+ | Homo sapiens |
| | | | | transporting, mitochondrial F0 | |
| | | | | complex, subunit c (subunit 9), | |
| | | | | isoform 1 | |
| 764 | AL080089 | 674 | 100 | hypothetical protein | Homo sapiens |
| 764 | X69907 | 674 | 100 | P1 gene for c subunit of human | Homo sapiens |
| | | | | mitochondrial ATP synthase | |
| 765 | AF100620_1 | 1492 | 100 | AF100620 transcription factor- | Homo sapiens |
| | | | | like protein MRGX | |
| 765 | AF167174_1 | 1492 | 100 | AF167174 MSL3-2 protein | Homo sapiens |
| 765 | AL049610 | 1492 | 100 | dJ1055C14.2 (KIAA0026 | Homo sapiens |
| | | | | (transcription factor-like | |
| | | | | protein MRGX)) | |
| 766 | AAH15557 | 1854 | 99 | BC015557 Unknown (protein for | Homo sapiens |
| | | | | MGC: 1567) | |
| 766 | BC021637 | 1140 | 66 | CD68 antigen | Mus musculus |
| 766 | S57235 | 1858 | 100 | CD68 | Homo sapiens |
| 767 | L27631 | 4010 | 100 | amyloid precursor-like protein 2 | Homo sapiens |
| 767 | S60099 | 4010 | 100 | APPH | Homo sapiens |
| 767 | Z22572 | 4010 | 100 | CDEI binding protein | Homo sapiens |
| 768 | AAH08850 | 1273 | 99 | BC008850 Unknown (protein for | Homo sapiens |
| | | | | MGC: 14308) | |
| 768 | AAH09599 | 1273 | 99 | BC009599 Unknown (protein for | Homo sapiens |
| | | | | MGC: 14690) | |
| 768 | L16558 | 1276 | 100 | ribosomal protein L7 | Homo sapiens |
| 769 | AF062482 | 845 | 99 | SDP3 | Mus musculus |
| 769 | AF062483 | 852 | 100 | SDP3 | Homo sapiens |
| 769 | AF520420 | 838 | 98 | sorting nexin 3 | Mus musculus |
| 770 | AF063657 | 6995 | 100 | vascular endothelial growth | Homo sapiens |
| | | | | factor receptor | |
| 770 | D28498 | 5882 | 82 | Fit-1 tyrosine kinase receptor | Rattus |
| | | | | | norvegicus |
| 770 | X51602 | 6989 | 99 | flt gene product (AA 1-1338) | Homo sapiens |
| 771 | AF081112 | 239 | 34 | ORF2 | Mus musculus |
| | | | | | domesticus |
| 771 | U83119 | 244 | 34 | ORF2 consensus sequence encoding | Rattus |
| | | | | endonuclease and reverse | norvegicus |
| | | | | transcriptase minus RNaseH | |
| 771 | U93572 | 336 | 42 | putative p150 | Homo sapiens |
| 772 | AF029729 | 3066 | 100 | neuralized | Homo sapiens |
| 772 | BC026336 | 3041 | 99 | neuralized-like (Drosophila) | Homo sapiens |
| 772 | U87864 | 3066 | 100 | neuralized homolog | Homo sapiens |
| 773 | AAH20782 | 643 | 100 | BC020782 glycoprotein hormones, | Homo sapiens |
| | | | | alpha polypeptide | |
| 773 | J00152 | 643 | 100 | glycoprotein alpha subunit | Homo sapiens |
| 773 | V00518 | 643 | 100 | chorionic gonadotropin | Homo sapiens |
| 774 | AAH02722 | 2070 | 100 | BC002722 guanine nucleotide | Homo sapiens |
| | | | | binding protein (G protein), | |
| | | | | alpha stimulating activity | |
| | | | | polypeptide 1 | |
| 774 | AF493897_1 | 2070 | 100 | AF493897 guanine nucleotide | Homo sapiens |
| | | | | binding protein alpha s long | |
| 774 | X04408 | 2070 | 100 | alpha-S2 (aa 1-394) | Homo sapiens |
| 775 | BC033714 | 1724 | 100 | heterogeneous nuclear | Homo sapiens |
| | | | | ribonucleoprotein A1 | |
| 775 | D86729 | 1724 | 100 | TIS | Mus musculus |
| 775 | M99167 | 1724 | 100 | RNA binding protein | Mus musculus |
| 776 | AF072836 | 1827 | 100 | Sox-like transcriptional factor | Homo sapiens |
| 776 | AF146223_1 | 620 | 100 | AF146223 HMG domain protein HMGX2 | Homo sapiens |
| 776 | AF288679 | 620 | 100 | smarcel-related protein | Homo sapiens |
| 777 | AAH21979 | 1360 | 100 | BC021979 ribosomal protein L7a | Homo sapiens |
| 777 | BC032533 | 1360 | 100 | ribosomal protein L7a | Homo sapiens |
| 777 | X15013 | 1360 | 100 | ribosomal protein L7a (AA 1-266) | Rattus |
| | | | | | Rattus |
| 778 | X51538 | 693 | 100 | ribosomal protein S24 (AA 1-133) | Rattus |
| | | | | | Rattus |
| 778 | X52445 | 693 | 100 | ribosomal protein S24 | Rattus |
| | | | | | norvegicus |
| 778 | X52658 | 693 | 100 | ribosomal protein S19 (AA 1-133) | Mesocricetus |
| | | | | | auratus |
| 779 | AAH01503 | 2789 | 100 | BC001503 prosaposin (variant | Homo sapiens |
| | | | | Gaucher disease and variant | |
| | | | | metachromatic leukodystrophy) | |
| 779 | AAH04275 | 2789 | 100 | BC004275 prosaposin (variant | Homo sapiens |
| | | | | Gaucher disease and variant | |
| | | | | metachromatic leukodystrophy) | |
| 779 | AAH07612 | 2789 | 100 | BC007612 prosaposin (variant | Homo sapiens |
| | | | | Gaucher disease and variant | |
| | | | | metachromatic leukodystrophy) | |
| 780 | AAH11776 | 1369 | 100 | BC011776 Similar to tropomyosin 2 | Homo sapiens |
| | | | | (beta) | |
| 780 | M74817 | 1369 | 100 | tropomyosin-1 | Homo sapiens |
| 780 | M75165 | 1369 | 100 | tropomyosin | Homo sapiens |
| 781 | AL023883 | 1173 | 99 | dJ404K8.1 (prolactin) | Homo sapiens |
| 781 | BC015850 | 1185 | 100 | similar to prolactin precursor | Homo sapiens |
| 781 | V00566 | 1185 | 100 | reading frame prolactin | Homo sapiens |
| 782 | AK012601 | 247 | 64 | unnamed protein product | Mus musculus |
| 782 | AK021409 | 286 | 69 | unnamed protein product | Mus musculus |
| 782 | AK075801 | 246 | 64 | unnamed protein product | Mus musculus |
| 783 | AF300943_1 | 9468 | 94 | AF300943 tyrosine phosphatase LAR | Mus musculus |
| 783 | L11586 | 9484 | 95 | leukocyte common antigen-related | Rattus |
| | | | | phosphatase | norvegicus |
| 783 | Y00815 | 9953 | 100 | put. LAR preprotein (AA −16 to | Homo sapiens |
| | | | | 1881) | |
| 784 | AAH16046 | 2739 | 100 | BC016046 ATP synthase, H+ | Homo sapiens |
| | | | | transporting, mitochondrial F1 | |
| | | | | complex, alpha subunit, isoform | |
| | | | | 1, cardiac muscle | |
| 784 | AAH19310 | 2739 | 100 | BC019310 ATP synthase, H+ | Homo sapiens |
| | | | | transporting, mitochondrial F1 | |
| | | | | complex, alpha subunit, isoform | |
| | | | | 1, cardiac muscle | |
| 784 | X65460 | 2739 | 100 | ATP synthase alpha subunit | Homo sapiens |
| 785 | AF237581_1 | 613 | 100 | AF237581 migration inhibitory | Homo sapiens |
| | | | | factor-related protein 14 variant P | |
| 785 | M26311 | 613 | 100 | cystic fibrosis antigen | Homo sapiens |
| 785 | X06233 | 613 | 100 | MRP-14 (AA 1-114) | Homo sapiens |
| 786 | AAH15492 | 523 | 96 | BC015492 Unknown (protein for | Homo sapiens |
| | | | | MGC: 8967) | |
| 786 | BC032626 | 280 | 64 | TLH29 protein precursor | Homo sapiens |
| 786 | X67325 | 556 | 100 | ISG12 | Homo sapiens |
| 787 | L00160 | 2135 | 100 | phosphoglycerate kinase | Homo sapiens |
| 787 | M11968 | 2135 | 100 | phosphoglycerate kinase | Homo sapiens |
| 787 | V00572 | 2135 | 100 | coding sequence | Homo sapiens |
| 788 | AF075575 | 11112 | 100 | dysferlin | Homo sapiens |
| 788 | AF188290_1 | 10355 | 93 | AF188290 dysferlin | Mus musculus |
| 788 | AK074104 | 7939 | 98 | FLJ00175 protein | Homo sapiens |
| 789 | AB052229 | 10414 | 100 | RNA polymerase II largest subunit | Cricetulus |
| 789 | D87294 | 10414 | 100 | RNA polymerase II largest subunit | Cricetulus |
| | | | | | griseus |
| 789 | X63564 | 10414 | 100 | RNA polymerase II largest subunit | Homo sapiens |
| 790 | AF070650 | 844 | 100 | F1FO-type ATP synthase subunit d | Homo sapiens |
| 790 | AF087135 | 844 | 100 | F1FO-type ATPase subunit d | Homo sapiens |
| 790 | BC038092 | 844 | 100 | ATP synthase, H+ transporting, | Homo sapiens |
| | | | | mitochondrial F0 complex, subunit d | |
| 791 | U01153 | 4046 | 97 | GRP94 | Canis |
| | | | | | familiaris |
| 791 | X15187 | 4112 | 100 | precursor polypeptide (AA −21 to | Homo sapiens |
| | | | | 782) | |
| 791 | Y09136 | 4038 | 97 | gp96/GRP94 | Sus scrofa |
| 792 | AF297036_1 | 670 | 100 | AF297036 ubiquitin fusion protein | Ophiophagus |
| | | | | | hannah |
| 792 | BC014772 | 670 | 100 | ubiquitin A-52 residue ribosomal | Mus musculus |
| | | | | protein fusion product 1 | |
| 792 | M24508 | 670 | 100 | ubiquitin | synthetic |
| | | | | | construct |
| 793 | AB047600 | 167 | 67 | hypothetical protein | Macaca |
| | | | | | fascicularis |
| 793 | AK097085 | 208 | 48 | unnamed protein product | Homo sapiens |
| 793 | X92485 | 165 | 68 | pval | Plasmodium |
| | | | | | vivax |
| 794 | AF100956 | 782 | 100 | ribosomal protein subunit S18 | Mus musculus |
| 794 | AF110520 | 782 | 100 | RPS18 | Mus musculus |
| 794 | M76763 | 782 | 100 | ribosomal protein | Mus musculus |
| 795 | BC025077 | 930 | 100 | RIKEN cDNA 2010203J19 gene | Mus musculus |
| 795 | L05092 | 930 | 100 | ribosomal protein L11 | Homo sapiens |
| 795 | X62146 | 930 | 100 | ribosomal protein L11 | Rattus |
| | | | | | Rattus |
| 796 | AB037890 | 6795 | 99 | pre-mRNA splicing factor SF3b 155 kDa | Mus musculus |
| | | | | subunit | |
| 796 | AF054284 | 6808 | 100 | spliceosomal protein SAP 155 | Homo sapiens |
| 796 | Y08997 | 6566 | 96 | 146 kDa nuclear protein | Xenopus |
| | | | | | laevis |
| 797 | AF072097 | 624 | 100 | beta-2 microglobulin | Homo sapiens |
| 797 | AY007153 | 624 | 100 | similar to Homo sapiens mRNA for | |
| | | | | beta 2-microglobulin with GenBank | |
| | | | | Accession Number AB021288 | |
| 797 | BC032589 | 624 | 100 | beta 2-microglobulin | Homo sapiens |
| 798 | AAH17839 | 1602 | 100 | BC017839 caspase 4, apoptosis- | Homo sapiens |
| | | | | related cysteine protease | |
| 798 | U28976 | 1602 | 100 | Mih1/TX isoform alpha | Homo sapiens |
| 798 | U28977 | 1769 | 100 | Mih1/TX isoform beta | Homo sapiens |
| 799 | AAH07967 | 974 | 100 | BC007967 Unknown (protein for | Homo sapiens |
| | | | | MGC: 14460) | |
| 799 | AAH12149 | 974 | 100 | BC012149 Unknown (protein for | Homo sapiens |
| | | | | MGC: 20365) | |
| 799 | BC031906 | 974 | 100 | similar to ribosomal protein L9; | Homo sapiens |
| | | | | 60S ribosomal protein L9 | |
| 800 | BC002145 | 745 | 100 | ribosomal protein S23 | Mus musculus |
| 800 | D14530 | 745 | 100 | ribosomal protein | Homo sapiens |
| 800 | X77398 | 745 | 100 | ribosomal protein S23 | Rattus |
| | | | | | norvegicus |
| 801 | AF227256_1 | 405 | 100 | AF227256 NEDD8 | Bos taurus |
| 801 | BC004625 | 398 | 98 | neural precursor cell expressed, | Mus musculus |
| | | | | developmentally down-regulated | |
| | | | | gene 8 | |
| 801 | D23662 | 405 | 100 | ubiquitin-like protein | Homo sapiens |
| 802 | U46034 | 2076 | 83 | stromelysin 3 | Rattus |
| | | | | | norvegicus |
| 802 | X57766 | 2679 | 100 | stromelysin-3 precursor | Homo sapiens |
| 802 | Z12604 | 2164 | 81 | stromelysin-3 | Mus musculus |
| 803 | AAH01734 | 499 | 100 | BC001734 protein translocation | Homo sapiens |
| | | | | complex beta | |
| 803 | AL137067 | 499 | 100 | bA13B9.2 (novel protein similar | Homo sapiens |
| | | | | to the beta subunit of transport | |
| | | | | protein SEC61) | |
| 803 | L25085 | 499 | 100 | Sec61-complex beta-subunit | Homo sapiens |
| 804 | AF165522_1 | 360 | 100 | AF165522 ras-related GTP-binding | Homo sapiens |
| | | | | protein 4b | |
| 804 | AF217985_1 | 360 | 100 | AF217985 unknown | Homo sapiens |
| 804 | AF498935_1 | 360 | 100 | AF498935 small GTP binding | Homo sapiens |
| | | | | protein RAB4B | |
| 805 | AF006602 | 5436 | 94 | histone deacetylase mHDA1 | Mus musculus |
| 805 | AF132608_1 | 5784 | 100 | AF132608 histone deacetylase 5 | Homo sapiens |
| 805 | AF207748_1 | 5431 | 94 | AF207748 histone deacetylase 5 | Mus musculus |
| 806 | AL050259 | 4009 | 100 | hypothetical protein | Homo sapiens |
| 806 | BC032681 | 4009 | 100 | RAB2, member RAS oncogene family- | Homo sapiens |
| | | | | like | |
| 806 | Z97184 | 4009 | 100 | cICF0811.4.1 (RAB2, member RAS | Homo sapiens |
| | | | | oncogene family-like, isoform 1) | |
| 807 | AF151855_1 | 1278 | 100 | AF151855 CGI-97 protein | Homo sapiens |
| 807 | AK001779 | 1214 | 96 | unnamed protein product | Homo sapiens |
| 807 | BC003849 | 1200 | 94 | RIKEN cDNA 4733401P19 gene | Mus musculus |
| 808 | AAH00234 | 1377 | 100 | BC000234 nicotinamide N- | Homo sapiens |
| | | | | methyltransferase | |
| 808 | U08021 | 1377 | 100 | nicotinamide N-methyltransferase | Homo sapiens |
| 808 | U20971 | 1377 | 100 | nicotinamide N-methyltransferase | Homo sapiens |
| 809 | AAH03137 | 1240 | 100 | BC003137 ribosomal protein S3 | Homo sapiens |
| 809 | BC034149 | 1240 | 100 | ribosomal protein S3 | Homo sapiens |
| 809 | U14992 | 1240 | 100 | ribosomal protein S3 | Homo sapiens |
| 810 | AF090891_1 | 3913 | 99 | AF090891 PRO0105 | Homo sapiens |
| 810 | AF268075_1 | 3913 | 99 | AF268075 TRAF6-binding protein | Homo sapiens |
| | | | | T6BP | |
| 810 | U33821 | 3924 | 100 | tax1-binding protein TXBP151 | Homo sapiens |
| 811 | AF186112_1 | 619 | 100 | AF186112 neurokinin B-like | Homo sapiens |
| | | | | protein ZNEUROK1 | |
| 811 | AF216586_1 | 619 | 100 | AF216586 neurokinin B | Homo sapiens |
| 811 | BC032145 | 619 | 100 | tachykinin 3 (neuromedin K, | Homo sapiens |
| | | | | neurokinin beta) | |
| 812 | AAH08405 | 2044 | 90 | BC008405 Unknown (protein for | Homo sapiens |
| | | | | MGC: 14528) | |
| 812 | M94891 | 2042 | 90 | pregnancy-specific beta-1 | Homo sapiens |
| | | | | glycoprotein | |
| 812 | U18467 | 2252 | 100 | pregnancy-specific beta 1- | Homo sapiens |
| | | | | glycoprotein 7 precursor | |
| 813 | AK002989 | 980 | 93 | unnamed protein product | Mus musculus |
| 813 | D38076 | 1041 | 99 | Ran-BP1 (Ran-binding protein 1) | Homo sapiens |
| 813 | X83617 | 1057 | 100 | RanBP1 | Homo sapiens |
| 814 | AF152604 | 3398 | 100 | bromodomain-containing protein | Homo sapiens |
| 814 | AF213969 | 3369 | 99 | bromodomain protein CELTIX1 | Homo sapiens |
| 814 | AJ271881 | 3398 | 100 | bromodomain containing protein | Homo sapiens |
| 815 | AK000496 | 327 | 65 | unnamed protein product | Homo sapiens |
| 815 | AK090511 | 328 | 65 | unnamed protein product | Homo sapiens |
| 815 | AX399963 | 319 | 62 | unnamed protein product | Homo sapiens |
| 816 | AK000917 | 2276 | 100 | unnamed protein product | Homo sapiens |
| 816 | BC025560 | 2156 | 94 | Similar to hypothetical protein | Mus musculus |
| | | | | FLJ10055 | |
| 816 | BC039867 | 2259 | 99 | hypothetical protein FLJ10055 | Homo sapiens |
| 817 | AB039886 | 1071 | 100 | down-regulated in gastric cancer | Homo sapiens |
| 817 | AY139182 | 995 | 100 | 18 kDa antrum mucosa protein | Homo sapiens |
| 817 | AY139184 | 995 | 100 | 18 kDa antrum mucosa protein | Homo sapiens |
| 818 | BC033721 | 3540 | 99 | Unknown (protein for MGC: 45264) | Homo sapiens |
| 818 | X82157 | 3543 | 100 | hevin | Homo sapiens |
| 818 | X86693 | 3504 | 99 | Hevin-like protein | Homo sapiens |
| 819 | M84472 | 1675 | 100 | 17-beta-hydroxysteroid | Homo sapiens |
| | | | | dehydrogenase | |
| 819 | U34879 | 1675 | 100 | 17-beta-hydroxysteroid | Homo sapiens |
| | | | | dehydrogenase | |
| 819 | X13440 | 1675 | 100 | 17-HSD protein (AA 1-328) | Homo sapiens |
| 820 | AAH12612 | 1812 | 99 | BC012612 Similar to cathepsin L | Homo sapiens |
| 820 | M20496 | 1816 | 100 | preprocathepsin L precursor | Homo sapiens |
| 820 | X12451 | 1816 | 100 | pro-(cathepsin L) | Homo sapiens |
| 821 | AAH12355 | 3449 | 100 | BC012355 Similar to solute | Homo sapiens |
| | | | | carrier family 6 | |
| | | | | (neurotransmitter transporter, | |
| | | | | creatine), member 8 | |
| 821 | U36341 | 3449 | 100 | creatine transporter | Homo sapiens |
| 821 | Z66539 | 3449 | 100 | creatine transporter | Homo sapiens |
| 822 | AF267852_1 | 749 | 100 | AF267852 placental protein 13- | Homo sapiens |
| | | | | like protein | |
| 822 | AF367430 | 571 | 100 | placental protein 13-like | Homo sapiens |
| 822 | BC022257 | 731 | 98 | Similar to placental protein 13- | Homo sapiens |
| | | | | like protein | |
| 823 | AB001636 | 4072 | 99 | ATP-dependent RNA helicase #46 | Homo sapiens |
| 823 | AF279891_1 | 4162 | 100 | AF279891 dead box protein 15 | Homo sapiens |
| 823 | BC035974 | 4162 | 100 | DEAD/H (Asp-Glu-Ala-Asp/His) box | Homo sapiens |
| | | | | polypeptide 15 | |
| 824 | AAH17321 | 687 | 100 | BC017321 Unknown (protein for | Homo sapiens |
| | | | | MGC: 29768) | |
| 824 | BC002079 | 687 | 100 | Similar to ribosomal protein S12 | Mus musculus |
| 824 | BC018362 | 687 | 100 | Unknown (protein for MGC: 19264) | Mus musculus |
| 825 | AAH20619 | 1123 | 99 | BC020619 glutathione S- | Homo sapiens |
| | | | | transferase A3 | |
| 825 | AF508266_1 | 1127 | 100 | AF508266 glutathione transferase | Homo sapiens |
| | | | | A3-3 | |
| 825 | AL121969 | 1127 | 100 | dJ214M20.1 (glutathione S- | Homo sapiens |
| | | | | transferase A3) | |
| 826 | AAH00359 | 923 | 100 | BC000359 signal peptidase complex | Homo sapiens |
| | | | | (18 kD) | |
| 826 | AAH14508 | 923 | 100 | BC014508 Similar to signal | Homo sapiens |
| | | | | peptidase complex (18 kD) | |
| 826 | AF061737 | 923 | 100 | microsomal signal peptidase | Homo sapiens |
| 827 | AF179890_1 | 598 | 100 | AF179890 DNA polymerase delta | Homo sapiens |
| | | | | smallest subunit p12 | |
| 827 | AF515709_1 | 492 | 83 | AF515709 small subunit DNA | Mus musculus |
| | | | | polymerase delta p12 | |
| 827 | BC028520 | 492 | 83 | Unknown (protein for MGC: 41275) | Mus musculus |
| 828 | AC005067_2 | 1340 | 73 | AC005067 Supported by Human EST | Homo sapiens |
| | | | | H08032.1 (NID: g872854), mouse EST | |
| | | | | AA870042.1 (NID: g2965487), and | |
| | | | | genscan | |
| 828 | AF226732_1 | 2286 | 100 | AF226732 NPD007 | Homo sapiens |
| 828 | BC018360 | 1199 | 68 | Unknown (protein for MGC: 18730) | Mus musculus |
| 829 | AAH19040 | 897 | 100 | BC019040 Unknown (protein for | Homo sapiens |
| | | | | MGC: 20769) | |
| 829 | D11428 | 826 | 100 | PMP-22(PAS-II/SR13/Gas-3) | Homo sapiens |
| 829 | L03203 | 826 | 100 | peripheral myelin protein 22 | Homo sapiens |
| 830 | AK023767 | 960 | 100 | unnamed protein product | Homo sapiens |
| 830 | AL080169 | 979 | 100 | hypothetical protein | Homo sapiens |
| 830 | BC016647 | 960 | 100 | Unknown (protein for MGC: 16844) | Homo sapiens |
| 831 | AF309646_1 | 1144 | 88 | AF309646 Rab6-like protein WTH3 | Homo sapiens |
| 831 | AF498941_1 | 1058 | 100 | AF498941 small GTP binding | Homo sapiens |
| | | | | protein RAB6C | |
| 831 | AL136727 | 1144 | 88 | hypothetical protein | Homo sapiens |
| 832 | AC005095_1 | 1373 | 100 | AC005095 pantophysin | Homo sapiens |
| 832 | S72481 | 1373 | 100 | pantophysin | Homo sapiens |
| 832 | X68194 | 1373 | 100 | h-Sp1 | Homo sapiens |
| 833 | D14665 | 3850 | 99 | KIAA0021 protein | Homo sapiens |
| 833 | U41765 | 3814 | 83 | metalloprotease/disintegrin/cysteine | Mus musculus |
| | | | | rich protein precursor | |
| 833 | U41766 | 4476 | 100 | metalloprotease/disintegrin/cysteine- | Homo sapiens |
| | | | | rich protein precursor | |
| 834 | U20770 | 1412 | 100 | cell surface glycoprotein | Homo sapiens |
| 834 | U67274 | 1412 | 100 | metastasis suppressor | Homo sapiens |
| 834 | X53795 | 1412 | 100 | inducible membrane protein | Homo sapiens |
| 835 | AF517934_1 | 2980 | 100 | AF517934 interleukin 2 receptor, | Homo sapiens |
| | | | | beta | |
| 835 | AL022314 | 2980 | 100 | dJ1170K4.3 (Interleukin 2 | Homo sapiens |
| | | | | Receptor, Beta (IL-2 Receptor, | |
| | | | | CD122 antigen)) | |
| 835 | BC025691 | 2980 | 100 | interleukin 2 receptor, beta | Homo sapiens |
| 836 | AF072242 | 2167 | 100 | methyl-CpG binding protein MBD2 | Homo sapiens |
| 836 | AF120994 | 2167 | 100 | methyl-CpG binding protein 2 | Homo sapiens |
| 836 | BC032638 | 2167 | 100 | methyl-CpG binding domain protein 2 | Homo sapiens |
| 837 | AAH00486 | 607 | 100 | BC000486 small nuclear | Homo sapiens |
| | | | | ribonucleoprotein D2 polypeptide | |
| | | | | (16.5 kD) | |
| 837 | AAH01930 | 607 | 100 | BC001930 small nuclear | Homo sapiens |
| | | | | ribonucleoprotein D2 polypeptide | |
| | | | | (16.5 kD) | |
| 837 | AC007191_1 | 607 | 100 | AC007191 SMD2_HUMAN; SNRNP CORE | Homo sapiens |
| | | | | PROTEIN D2; SM-D2 | |
| 838 | AAH18180 | 870 | 100 | BC018180 CGI-121 protein | Homo sapiens |
| 838 | AF151879_1 | 870 | 100 | AF151879 CGI-121 protein | Homo sapiens |
| 838 | BC029492 | 870 | 100 | CGI-121 protein | Homo sapiens |
| 839 | U52373 | 4063 | 99 | MNB | Homo sapiens |
| 839 | U58497 | 4037 | 99 | mp86 | Mus musculus |
| 839 | X79769 | 4052 | 99 | Dual Specificity Yak1-related | Rattus |
| | | | | Kinase (Dyrk) | norvegicus |
| 853 | AF090949 | 1710 | 97 | HBV-X associated protein 2 | Cercopithecus |
| | | | | | aethiops |
| 853 | U31913 | 1736 | 99 | HBV-X associated protein | Homo sapiens |
| 853 | U78521 | 1740 | 100 | immunophilin homolog ARA9 | Homo sapiens |
| 854 | AAH12333 | 1168 | 100 | BC012333 Unknown (protein for | Homo sapiens |
| | | | | MGC: 2084) | |
| 854 | AF187981_1 | 1168 | 100 | AF187981 delta7-sterol-C5- | Homo sapiens |
| | | | | desaturase | |
| 854 | D85181 | 1273 | 100 | fungal sterol-C5-desaturase | Homo sapiens |
| | | | | homolog | |
| 856 | A84511 | 1017 | 99 | unnamed protein product | unidentified |
| 856 | AF498970_1 | 1016 | 100 | AF498970 small GTP binding | Homo sapiens |
| | | | | protein RhoA | |
| 856 | AX427410 | 1017 | 99 | unnamed protein product | unidentified |
| 857 | AAH04312 | 563 | 94 | BC004312 insulin-like growth | Homo sapiens |
| | | | | factor binding protein 2 (36 kD) | |
| 857 | AAH09902 | 563 | 94 | BC009902 Unknown (protein for | Homo sapiens |
| | | | | MGC: 2339) | |
| 857 | AAH12769 | 563 | 94 | BC012769 Unknown (protein for | Homo sapiens |
| | | | | MGC: 16256) | |
| 858 | AAH02434 | 430 | 100 | BC002434 p8 protein (candidate of | Homo sapiens |
| | | | | metastasis 1) | |
| 858 | AF069074 | 430 | 100 | P8 protein | Homo sapiens |
| 858 | AF135266_1 | 430 | 100 | AF135266 p8 protein homolog | Homo sapiens |
| 859 | AB047892 | 301 | 84 | hypothetical protein | Macaca |
| | | | | | fascicularis |
| 860 | AAH01199 | 833 | 99 | BC001199 Similar to glucose | Homo sapiens |
| | | | | regulated protein, 58 kDa | |
| 860 | AJ440721 | 833 | 99 | thioredoxin related protein | Homo sapiens |
| 860 | AL834423 | 833 | 99 | hypothetical protein | Homo sapiens |
| 861 | AAH00866 | 1124 | 100 | BC000866 tissue inhibitor of | Homo sapiens |
| | | | | metalloproteinase 1 (erythroid | |
| | | | | potentiating activity, | |
| | | | | collagenase inhibitor) | |
| 861 | M59906 | 1124 | 100 | collagenase inhibitor | Homo sapiens |
| 861 | X03124 | 1124 | 100 | precursor | Homo sapiens |
| 862 | AAH00518 | 1120 | 98 | BC000518 Similar to brain acid- | Homo sapiens |
| | | | | soluble protein 1 | |
| 862 | AF039656 | 1105 | 96 | neuronal tissue-enriched acidic | Homo sapiens |
| | | | | protein | |
| 862 | U92535 | 818 | 75 | neuronal axonal membrane protein | Bos taurus |
| 863 | AAH05931 | 733 | 100 | BC005931 hemoglobin, alpha 2 | Homo sapiens |
| 863 | AAH08572 | 733 | 100 | BC008572 hemoglobin, alpha 2 | Homo sapiens |
| 863 | BC032122 | 733 | 100 | hemoglobin, alpha 2 | Homo sapiens |
| 864 | AF347006 | 2436 | 91 | cytochrome c oxidase subunit I | Homo sapiens |
| 864 | AF382012 | 2424 | 92 | cytochrome c oxidase subunit I | Homo sapiens |
| 864 | AF382013 | 2424 | 92 | cytochrome c oxidase subunit I | Homo sapiens |
| 865 | AF242729_1 | 1242 | 100 | AF242729 HT022 | Homo sapiens |
| 865 | AJ245874 | 1242 | 100 | putative ATG/GTP binding protein | Homo sapiens |
| 865 | AJ250344 | 1242 | 100 | hypothetical protein | Homo sapiens |
| 866 | AAH17050 | 1367 | 100 | BC017050 2,3-bisphosphoglycerate | Homo sapiens |
| | | | | mutase | |
| 866 | M23068 | 1367 | 100 | 2,3-bisphosphoglycerate | Homo sapiens |
| 866 | X04327 | 1367 | 100 | 2,3 biphosphoglycerated mutase | Homo sapiens |
| | | | | (AA 1-259) | |
| 867 | AAH02334 | 921 | 100 | BC002334 DAZ associated protein 2 | Homo sapiens |
| 867 | AAH07900 | 921 | 100 | BC007900 DAZ associated protein 2 | Homo sapiens |
| 867 | D31767 | 921 | 100 | KIAA0058 | Homo sapiens |
| 868 | AAH09571 | 308 | 98 | BC009571 Similar to stimulated by | Homo sapiens |
| | | | | retinoic acid 13 | |
| 868 | U95006 | 309 | 98 | D9 splice variant A | Homo sapiens |
| 868 | U95007 | 273 | 98 | D9 splice variant B | Homo sapiens |
| 869 | AAH11674 | 753 | 92 | BC011674 Similar to procollagen- | Homo sapiens |
| | | | | lysine, 2-oxoglutarate 5- | |
| | | | | dioxygenase 3 | |
| 869 | AC004876_4 | 753 | 92 | AC004876 lysyl hydroxylase 3 | Homo sapiens |
| 869 | AF207069 | 753 | 92 | lysyl hydroxylase 3 | Homo sapiens |
| 870 | AAH16765 | 1778 | 99 | BC016765 Unknown (protein for | Homo sapiens |
| | | | | MGC: 22663) | |
| 870 | AF153882_1 | 1785 | 100 | AF153882 reversion-induced LIM | Homo sapiens |
| | | | | protein | |
| 870 | AF154335_1 | 1785 | 100 | AF154335 reversion-induced LIM | Homo sapiens |
| | | | | protein | |
| 871 | AF023268 | 1546 | 95 | metaxin | Homo sapiens |
| 871 | BC023071 | 1424 | 87 | metaxin | Mus musculus |
| 871 | U46920 | 1546 | 95 | metaxin | Homo sapiens |
| 872 | AF193053_1 | 575 | 100 | AF193053 unknown | Homo sapiens |
| 873 | AF318351_1 | 3211 | 100 | AF318351 unknown | Homo sapiens |
| 873 | AF318377_1 | 3211 | 100 | AF318377 unknown | Homo sapiens |
| 873 | AX106709 | 3211 | 100 | unnamed protein product | Homo sapiens |
| 874 | AL357374 | 2678 | 100 | bA353C18.3.2 (splicing factor | Homo sapiens |
| | | | | CC1.3, isoform 2 (CC1.4)) | |
| 874 | AY061882 | 2663 | 99 | transcription coactivator CAPER | Mus musculus |
| 874 | L10911 | 2678 | 100 | splicing factor | Homo sapiens |
| 875 | AAH08807 | 2806 | 98 | BC008807 keratin 6A | Homo sapiens |
| 875 | AAH14152 | 2809 | 98 | BC014152 Similar to keratin 6A | Homo sapiens |
| 875 | L42583 | 2806 | 98 | keratin type II | Homo sapiens |
| 876 | AK000496 | 301 | 72 | unnamed protein product | Homo sapiens |
| 876 | AK025116 | 271 | 67 | unnamed protein product | Homo sapiens |
| 876 | AK090511 | 282 | 60 | unnamed protein product | Homo sapiens |
| 877 | AAH16842 | 2431 | 99 | BC016842 Unknown (protein for | Homo sapiens |
| | | | | MGC: 10135) | |
| 877 | AK027643 | 2435 | 100 | unnamed protein product | Homo sapiens |
| 877 | AL834398 | 2529 | 99 | hypothetical protein | Homo sapiens |
| 878 | AAH02563 | 1624 | 100 | BC002563 Similar to zinc/iron | Homo sapiens |
| | | | | regulated transporter-like | |
| 878 | AAH07886 | 1624 | 100 | BC007886 Unknown (protein for | Homo sapiens |
| | | | | MGC: 14180) | |
| 878 | AAH14303 | 1624 | 100 | BC014303 Unknown (protein for | Homo sapiens |
| | | | | MGC: 16418) | |
| 879 | AF056732 | 3342 | 100 | prostaglandin transporter | Homo sapiens |
| 879 | BC041140 | 3478 | 99 | solute carrier family 21 | Homo sapiens |
| | | | | (prostaglandin transporter), | |
| | | | | member 2 | |
| 879 | U70867 | 3342 | 100 | prostaglandin transporter hPGT | Homo sapiens |
| 880 | AAH15206 | 335 | 100 | BC015206 guanine nucleotide | Homo sapiens |
| | | | | binding protein 10 | |
| 880 | AAH16319 | 335 | 100 | BC016319 guanine nucleotide | Homo sapiens |
| | | | | binding protein 10 | |
| 880 | AF493877_1 | 335 | 100 | AF493877 guanine nucleotide | Homo sapiens |
| | | | | binding protein gamma 10 | |
| 881 | BC010572 | 1333 | 79 | Jun proto-oncogene related gene | Mus musculus |
| | | | | d1 | |
| 881 | X51346 | 1517 | 99 | jun-D gene product (AA 1-303) | Homo sapiens |
| 881 | X56681 | 1716 | 98 | junD protein | Homo sapiens |
| 882 | AY081680 | 693 | 100 | chlorophyll a/b-binding protein | Arabidopsis |
| | | | | CP29 | thaliana |
| 882 | AY133566 | 693 | 100 | At5g01530/F7A7_50 | Arabidopsis |
| | | | | | thaliana |
| 882 | BT000363 | 693 | 100 | chlorophyll a/b-binding protein | Arabidopsis |
| | | | | CP29 | thaliana |
| 883 | AY086905 | 622 | 100 | photosystem II type I | Arabidopsis |
| | | | | chlorophyll a/b binding protein, | thaliana |
| | | | | putative | |
| 883 | AY128345 | 622 | 100 | photosystem II type I chlorophyll | Arabidopsis |
| | | | | a/b binding protein, putative | thaliana |
| 883 | BT000726 | 622 | 100 | putative photosystem II type I | Arabidopsis |
| | | | | chlorophyll a/b binding protein | thaliana |
| 884 | AK074519 | 1524 | 97 | unnamed protein product | Homo sapiens |
| 884 | AK074523 | 1524 | 97 | unnamed protein product | Homo sapiens |
| 884 | AX118867 | 1543 | 100 | unnamed protein product | Homo sapiens |
| 885 | AY050935 | 572 | 100 | putative photosystem II type I | Arabidopsis |
| | | | | chlorophyll a/b binding protein | thaliana |
| 885 | AY058180 | 572 | 100 | At1g29930/F1N18_23 | Arabidopsis |
| | | | | | thaliana |
| 885 | AY091169 | 572 | 100 | putative chlorophyll a/b-binding | Arabidopsis |
| | | | | protein | thaliana |
| 886 | AF178954 | 2149 | 88 | monocarboxylate transporter 4 | Mus musculus |
| 886 | U81800 | 2399 | 100 | monocarboxylate transporter | Homo sapiens |
| 886 | U87627 | 2164 | 88 | putative monocarboxylate | Rattus |
| | | | | transporter | norvegicus |
| 887 | AAH18345 | 173 | 100 | BC018345 Unknown (protein for | Homo sapiens |
| | | | | MGC: 8802) | |
| 887 | AF237776_1 | 173 | 100 | AF237776 MAP kinase-interacting | Homo sapiens |
| | | | | kinase 2b | |
| 887 | AL137565 | 173 | 100 | hypothetical protein | Homo sapiens |
| 888 | AF006013 | 3773 | 99 | dishevelled 3 | Homo sapiens |
| 888 | BC032459 | 3784 | 99 | dishevelled, dsh homolog 3 | Homo sapiens |
| | | | | (Drosophila) | |
| 888 | U75651 | 3806 | 100 | dishevelled 3 | Homo sapiens |
| 889 | AK000268 | 2659 | 100 | unnamed protein product | Homo sapiens |
| 889 | AK053678 | 1598 | 71 | unnamed protein product | Mus musculus |
| 889 | L00193 | 1342 | 58 | epidermal keratin subunit I | Mus musculus |
| 890 | AX354357 | 840 | 100 | unnamed protein product | Homo sapiens |
| 890 | BC002198 | 243 | 42 | Similar to hypothetical protein | Mus musculus |
| | | | | FLJ20093 | |
| 890 | BC039642 | 658 | 75 | Unknown (protein for | Mus musculus |
| | | | | IMAGE: 5352548) | |
| 891 | AF012348 | 962 | 92 | smooth muscle gamma actin | Gallus |
| | | | | | gallus |
| 891 | BC002042 | 962 | 92 | Similar to actin, gamma 2, smooth | Mus musculus |
| | | | | muscle, enteric | |
| 891 | S63494 | 962 | 92 | gamma-actin | Gallus |
| | | | | | gallus |
| 892 | AAH13867 | 2057 | 100 | BC013867 Similar to palladin | Homo sapiens |
| 892 | AB023209 | 2064 | 99 | KIAA0992 protein | Homo sapiens |
| 892 | AF151909_1 | 2161 | 100 | AF151909 CGI-151 protein | Homo sapiens |
| 893 | AF287271_1 | 714 | 100 | AF287271 ribosomal protein L23 | Mus musculus |
| 893 | AF401577_1 | 714 | 100 | AF401577 ribosomal protein L23 | Ictalurus |
| | | | | | punctatus |
| 893 | BC025918 | 714 | 100 | ribosomal protein L23 | Mus musculus |
| 894 | U43916 | 817 | 100 | TMP | Homo sapiens |
| 894 | Y07909 | 817 | 100 | progression associated protein | Homo sapiens |
| 894 | Z50751 | 817 | 100 | B4B | Homo sapiens |
| 895 | AAH09647 | 2778 | 99 | BC009647 Unknown (protein for | Homo sapiens |
| | | | | MGC: 5096) | |
| 895 | M13078 | 2773 | 99 | alkaline phosphatase precursor | Homo sapiens |
| 895 | M19159 | 2780 | 100 | placental alkaline phosphatase-1 | Homo sapiens |
| 896 | AF326591 | 2240 | 100 | fenestrated-endothelial linked | Homo sapiens |
| | | | | structure protein | |
| 896 | AF348827 | 2232 | 99 | PV1 protein | Homo sapiens |
| 896 | AL834363 | 2304 | 99 | hypothetical protein | Homo sapiens |
| 897 | AAH11686 | 5877 | 100 | BC011686 damage-specific DNA | Homo sapiens |
| | | | | binding protein 1 (127 kD) | |
| 897 | L40326 | 5877 | 100 | X-associated protein 1 | Homo sapiens |
| 897 | U18299 | 5877 | 100 | DDBa p127 | Homo sapiens |
| 898 | U66661 | 1330 | 100 | GABA-A receptor epsilon subunit | Homo sapiens |
| 898 | Y07637 | 1330 | 100 | putative GABA-gated chloride | Homo sapiens |
| | | | | channel | |
| 898 | Y09765 | 1330 | 100 | GABA receptor epsilon subunit | Homo sapiens |
| 899 | AF380181_1 | 11506 | 99 | AF380181 SON DNA binding protein | Homo sapiens |
| | | | | isoform C | |
| 899 | AF380184_1 | 12193 | 99 | AF380184 SON DNA binding protein | Homo sapiens |
| | | | | isoform F | |
| 899 | AY026895 | 11871 | 97 | NREBP | Homo sapiens |
| 900 | A20470 | 2142 | 99 | preA-PAI-2 | synthetic |
| | | | | | construct |
| 900 | AAH12609 | 2148 | 100 | BC012609 Similar to serine (or | Homo sapiens |
| | | | | cysteine) proteinase inhibitor, | |
| | | | | clade B (ovalbumin), member 2 | |
| 900 | Y00630 | 2139 | 99 | PAI-2 precursor (AA −22 to 393) | Homo sapiens |
| 901 | AB037819 | 7936 | 100 | KIAA1398 protein | Homo sapiens |
| 901 | AL132765 | 5785 | 95 | bA462D18.3.2 (ribosome binding | Homo sapiens |
| | | | | protein 1 (dog 180 kDa homolog) | |
| | | | | (KIAA1398) (isoform 2)) | |
| 901 | X87224 | 6518 | 84 | ribosome receptor | Canis |
| | | | | | familiaris |
| 902 | AAH00716 | 2646 | 98 | BC000716 seryl-tRNA synthetase | Homo sapiens |
| 902 | D49914 | 2646 | 98 | Seryl tRNA Synthetase | Homo sapiens |
| 902 | X91257 | 2649 | 98 | seryl-tRNA synthetase | Homo sapiens |
| 903 | D29640 | 4698 | 57 | KIAA0051 | Homo sapiens |
| 903 | L33075 | 4698 | 57 | ras GTPase-activating-like | Homo sapiens |
| | | | | protein | |
| 903 | U51903 | 8003 | 100 | RasGAP-related protein | Homo sapiens |
| 904 | AAH17338 | 2576 | 99 | BC017338 fucosidase, alpha-L-1, | Homo sapiens |
| | | | | tissue | |
| 904 | M29877 | 2581 | 100 | alpha-L-fucosidase precursor (EC | Homo sapiens |
| | | | | 3.2.1.5) | |
| 904 | M80815 | 2576 | 99 | alpha-L-fucosidase | Homo sapiens |
| 905 | AAH03067 | 3601 | 100 | BC003067 growth arrest and DNA- | Homo sapiens |
| | | | | damage-inducible 34 | |
| 905 | AK001361 | 3568 | 99 | unnamed protein product | Homo sapiens |
| 905 | U83981 | 3601 | 100 | apoptosis associated protein | Homo sapiens |
| 906 | AAH02717 | 379 | 77 | BC002717 Similar to chorionic | Homo sapiens |
| | | | | somatomammotropin hormone 1 | |
| | | | | (placental lactogen) | |
| 906 | AAH05921 | 379 | 77 | BC005921 chorionic | Homo sapiens |
| | | | | somatomammotropin hormone 1 | |
| | | | | (placental lactogen) | |
| 906 | AAH20756 | 379 | 77 | BC020756 chorionic | Homo sapiens |
| | | | | somatomammotropin hormone 1 | |
| | | | | (placental lactogen) | |
| 907 | M57939 | 2349 | 100 | ribonucleoprotein antigen | Homo sapiens |
| 907 | X04654 | 3252 | 100 | 70 K protein (AA 1-614) | Homo sapiens |
| 907 | X84841 | 2349 | 100 | 68 kDa (U1) ribonucleoprotein | Homo sapiens |
| 908 | AAH09945 | 3737 | 99 | BC009945 Unknown (protein for | Homo sapiens |
| | | | | IMAGE: 4123572) | |
| 908 | AB018289 | 5502 | 100 | KIAA0746 protein | Homo sapiens |
| 908 | AK030487 | 4620 | 87 | unnamed protein product | Mus musculus |
| 909 | AJ422147 | 664 | 100 | C15 protein | Homo sapiens |
| 909 | AX352692 | 664 | 100 | unnamed protein product | Homo sapiens |
| 909 | AX357061 | 664 | 100 | unnamed protein product | Homo sapiens |
| 910 | AF251294_1 | 375 | 90 | AF251294 hCLA-iso | Homo sapiens |
| 910 | AL834432 | 375 | 90 | hypothetical protein | Homo sapiens |
| 910 | AY037150 | 375 | 90 | hypothetical protein SB138 | Homo sapiens |
| 911 | AAH01501 | 1520 | 100 | BC001501 Unknown (protein for | Homo sapiens |
| | | | | MGC: 2233) | |
| 911 | AAH14078 | 1520 | 100 | BC014078 cyclin D1 (PRAD1: | Homo sapiens |
| | | | | parathyroid adenomatosis 1) | |
| 911 | BC025302 | 1520 | 100 | cyclin D1 (PRAD1: parathyroid | Homo sapiens |
| | | | | adenomatosis 1) | |
| 912 | BC036285 | 1936 | 100 | protein kinase, cAMP-dependent, | Homo sapiens |
| | | | | regulatory, type I, alpha (tissue | |
| | | | | specific extinguisher 1) | |
| 912 | M18468 | 1936 | 100 | cAMP-dependent protein kinase | Homo sapiens |
| | | | | regulatory subunit type 1 | |
| 912 | M33336 | 1936 | 100 | cAMP-dependent protein kinase | Homo sapiens |
| | | | | type I-alpha subunit | |
| 913 | BC024165 | 600 | 100 | polymerase (RNA) II (DNA | Homo sapiens |
| | | | | directed) polypeptide J (13.3 kD) | |
| 913 | X82385 | 600 | 100 | RNA polymerase II subunit | Homo sapiens |
| 913 | X98433 | 600 | 100 | RNA polymerase II subunit | Homo sapiens |
| 914 | AL138761 | 8033 | 99 | bA16H23.2 (collagen, type XVII, | Homo sapiens |
| | | | | alpha 1 (BP180)) | |
| 914 | M91669 | 8228 | 100 | autoantigen | Homo sapiens |
| 914 | U76604 | 8037 | 99 | 180 kDa bullous pemphigoid | Homo sapiens |
| | | | | antigen 2/type XVII collagen | |
| 915 | AAH03575 | 2373 | 100 | BC003575 mannosyl (alpha-1,3-)- | Homo sapiens |
| | | | | glycoprotein beta-1,2-N- | |
| | | | | acetylglucosaminyltransferase | |
| 915 | M55621 | 2363 | 99 | N-acetylglucosaminyltransferase I | Homo sapiens |
| 915 | M61829 | 2373 | 100 | alpha-1,3-mannosyl-glycoprotein | Homo sapiens |
| | | | | beta-1,2-N- | |
| | | | | acetylglucosaminyltransferase | |
| 916 | L13923 | 16639 | 99 | fibrillin | Homo sapiens |
| 916 | L28748 | 16362 | 97 | putative | Bos taurus |
| 916 | X63556 | 17359 | 100 | fibrillin | Homo sapiens |
| 917 | AAH02576 | 3661 | 100 | BC002576 matrix metalloproteinase | Homo sapiens |
| | | | | 2 (gelatinase A, 72 kD gelatinase, | |
| | | | | 72 kD type IV collagenase) | |
| 917 | J03210 | 3584 | 100 | collagenase type IV precursor | Homo sapiens |
| 917 | M55593 | 3661 | 100 | type IV collagenase | Homo sapiens |
| 918 | AAH22087 | 2466 | 99 | BC022087 Similar to CD36 antigen | Homo sapiens |
| | | | | (collagen type I receptor, | |
| | | | | thrombospondin receptor)-like 1 | |
| 918 | AF467889_1 | 2380 | 86 | AF467889 high density lipoprotein | Sus scrofa |
| | | | | receptor SR-BI | |
| 918 | Z22555 | 2684 | 100 | CLA-1 | Homo sapiens |
| 919 | AF106202_1 | 1249 | 100 | AF106202 endothelial cell protein | Homo sapiens |
| | | | | C receptor precursor | |
| 919 | AF375468_1 | 1249 | 100 | AF375468 endothelial protein C | Homo sapiens |
| | | | | receptor | |
| 919 | X89079 | 1249 | 100 | centrocyclin | Homo sapiens |
| 920 | AF153603_1 | 666 | 100 | AF153603 TSC-22 related protein | Homo sapiens |
| 920 | AF183393_1 | 666 | 100 | AF183393 TSC-22-like Protein | Homo sapiens |
| 920 | AF228339 | 666 | 100 | glucocorticoid-induced GILZ | Homo sapiens |
| 921 | AF111168 | 3586 | 99 | unknown | Homo sapiens |
| 921 | AK075445 | 2876 | 99 | unnamed protein product | Homo sapiens |
| 921 | AX136231 | 2876 | 99 | unnamed protein product | Homo sapiens |
| 922 | AAH11234 | 1220 | 100 | BC011234 Similar to splicing | Homo sapiens |
| | | | | factor 30, survival of motor | |
| | | | | neuron-related | |
| 922 | AF083385 | 1220 | 100 | 30 kDa splicing factor; SPF 30 | Homo sapiens |
| 922 | AF107463 | 1220 | 100 | splicing factor | Homo sapiens |
| 923 | AAH10240 | 1921 | 100 | BC010240 cathepsin B | Homo sapiens |
| 923 | L16510 | 1912 | 99 | cathepsin B | Homo sapiens |
| 923 | M14221 | 1915 | 99 | preprocathepsin B | Homo sapiens |
| 924 | AK056033 | 495 | 97 | unnamed protein product | Homo sapiens |
| 925 | AE003588 | 205 | 22 | CG2839-PA | Drosophila |
| | | | | | melanogaster |
| 925 | AE014836_21 | 231 | 26 | AE014836 hypothetical protein | Plasmodium |
| | | | | | falciparum |
| | | | | | 3D7 |
| 925 | AY052127 | 202 | 22 | SD07741p | Drosophila |
| | | | | | melanogaster |
| 926 | AF222743_1 | 2415 | 100 | AF222743 Sec61 alpha-1 | Mus musculus |
| 926 | BC003707 | 2415 | 100 | sec61 homolog | Mus musculus |
| 926 | M96630 | 2415 | 100 | sec61-like protein | Rattus sp. |
| 927 | AF332086_1 | 3193 | 96 | AF332086 Rad21 | Mus musculus |
| 927 | D38551 | 3284 | 100 | The ha1237 gene product is | Homo sapiens |
| | | | | related to S. pombe rad21 gene | |
| | | | | product. | |
| 927 | X98294 | 3275 | 99 | HR21spA | Homo sapiens |
| 928 | AAH16325 | 925 | 100 | BC016325 ADP-ribosylation factor 4 | Homo sapiens |
| 928 | AF493883_1 | 925 | 100 | AF493883 ADP-ribosylation factor | Homo sapiens |
| | | | | protein 4 | |
| 928 | BC022866 | 925 | 100 | ADP-ribosylation factor 4 | Homo sapiens |
| 929 | AAH18201 | 763 | 100 | BC018201 Unknown (protein for | Homo sapiens |
| | | | | MGC: 21505) | |
| 929 | AF217965_1 | 763 | 100 | AF217965 unknown | Homo sapiens |
| 929 | AF217972_1 | 763 | 100 | AF217972 unknown | Homo sapiens |
| 930 | AAH00211 | 1162 | 100 | BC000211 eukaryotic translation | Homo sapiens |
| | | | | elongation factor 1 beta 2 | |
| 930 | AAH04931 | 1162 | 100 | BC004931 eukaryotic translation | Homo sapiens |
| | | | | elongation factor 1 beta 2 | |
| 930 | X60489 | 1162 | 100 | elongation factor-1-beta | Homo sapiens |
| 931 | D82984 | 1758 | 93 | C/EBP alpha protein | Bos taurus |
| 931 | U34070 | 1857 | 96 | CCAAT/enhancer binding protein | Homo sapiens |
| | | | | alpha | |
| 931 | Y11525 | 1927 | 100 | CCAAT/enhancer binding protein | Homo sapiens |
| | | | | alpha | |
| 932 | AK025116 | 341 | 71 | unnamed protein product | Homo sapiens |
| 932 | AK090511 | 358 | 72 | unnamed protein product | Homo sapiens |
| 932 | AK096373 | 341 | 70 | unnamed protein product | Homo sapiens |
| 933 | AAH13963 | 2458 | 100 | BC013963 Similar to adenylyl | Homo sapiens |
| | | | | cyclase-associated protein | |
| 933 | AAH17196 | 2458 | 100 | BC017196 adenylyl cyclase- | Homo sapiens |
| | | | | associated protein | |
| 933 | M98474 | 2458 | 100 | CAP protein | Homo sapiens |
| 934 | AAH02464 | 2175 | 99 | BC002464 coagulation factor II | Homo sapiens |
| | | | | (thrombin) receptor | |
| 934 | AF391809_1 | 2179 | 99 | AF391809 coagulation factor II | Homo sapiens |
| | | | | (thrombin) receptor | |
| 934 | M62424 | 2187 | 100 | thrombin receptor | Homo sapiens |
| 935 | AF085250 | 759 | 100 | calmodulin | Perca |
| | | | | | flavescens |
| 935 | D83350 | 759 | 100 | calmodulin | Anas |
| | | | | | platyrhynchos |
| 935 | K01945 | 759 | 100 | calmodulin (cDNA clone 71) | Xenopus |
| | | | | | laevis |
| 936 | AL050265 | 2193 | 100 | hypothetical protein | Homo sapiens |
| 936 | BC033475 | 2128 | 96 | similar to TAR DNA-binding | Mus musculus |
| | | | | protein-43 (TDP-43) | |
| 936 | U23731 | 2193 | 100 | TAR DNA-binding protein-43 | Homo sapiens |
| 937 | AAH01495 | 1673 | 99 | BC001495 chromosome 1 open | Homo sapiens |
| | | | | reading frame 8 | |
| 937 | AAH16374 | 1697 | 100 | BC016374 chromosome 1 open | Homo sapiens |
| | | | | reading frame 8 | |
| 937 | AF290615_1 | 1673 | 99 | AF290615 liver membrane-bound | Homo sapiens |
| | | | | protein | |
| 938 | AK000385 | 329 | 65 | unnamed protein product | Homo sapiens |
| 938 | AK092450 | 360 | 69 | unnamed protein product | Homo sapiens |
| 938 | AK097266 | 329 | 74 | unnamed protein product | Homo sapiens |
| 939 | AAH00642 | 683 | 99 | BC000642 MY047 protein | Homo sapiens |
| 939 | AF063605 | 688 | 100 | brain my047 protein | Homo sapiens |
| 939 | AX136119 | 683 | 99 | unnamed protein product | Homo sapiens |
| 940 | AF035947_1 | 1394 | 100 | AF035947 cytokine-inducible | Homo sapiens |
| | | | | inhibitor of signalling type 1b | |
| 940 | AF132297_1 | 1332 | 99 | AF132297 cytokine-inducible SH2- | Homo sapiens |
| | | | | containing protein | |
| 940 | BC031590 | 1321 | 100 | cytokine inducible SH2-containing | Homo sapiens |
| | | | | protein | |
| 941 | AF151018_1 | 955 | 100 | AF151018 HSPC184 | Homo sapiens |
| 941 | AK012047 | 951 | 99 | unnamed protein product | Mus musculus |
| 941 | AK075227 | 955 | 100 | unnamed protein product | Homo sapiens |
| 942 | AK025116 | 322 | 70 | unnamed protein product | Homo sapiens |
| 942 | AK090511 | 302 | 63 | unnamed protein product | Homo sapiens |
| 942 | AK090929 | 308 | 71 | unnamed protein product | Homo sapiens |
| 943 | AAH09357 | 1052 | 100 | BC009357 transgelin 2 | Homo sapiens |
| 943 | AY007127 | 1157 | 100 | similar to Homo sapiens mRNA for | |
| | | | | KIAA0120 gene with GenBank | |
| | | | | Accession Number D21261.1 | |
| 943 | D21261 | 1052 | 100 | similar to human 22 kDa, SM22 mRNA | Homo sapiens |
| | | | | (HUM22SM). | |
| 945 | AAH14006 | 260 | 89 | BC014006 Similar to 6- | Homo sapiens |
| | | | | phosphogluconolactonase | |
| 945 | AJ243972 | 260 | 89 | 6-phosphogluconolactonase | Homo sapiens |
| 945 | BC006594 | 243 | 80 | RIKEN cDNA 1110030K05 gene | Mus musculus |
| 946 | AAH03125 | 2055 | 99 | BC003125 Unknown (protein for | Homo sapiens |
| | | | | MGC: 3222) | |
| 946 | AK027877 | 2055 | 99 | unnamed protein product | Homo sapiens |
| 946 | AL136916 | 2055 | 99 | hypothetical protein | Homo sapiens |
| 947 | AAH03681 | 2956 | 100 | BC003681 Unknown (protein for | Homo sapiens |
| | | | | IMAGE: 3453235) | |
| 947 | AL096828 | 3008 | 100 | dJ963E22.1 (Novel protein similar | Homo sapiens |
| | | | | to NY-REN-2 Antigen) | |
| 947 | AX400070 | 2878 | 96 | unnamed protein product | Homo sapiens |
| 948 | AF161372_1 | 872 | 100 | AF161372 HSPC254 | Homo sapiens |
| 949 | AAH11714 | 476 | 100 | BC011714 Similar to heterogeneous | Homo sapiens |
| | | | | nuclear ribonucleoprotein D-like | |
| 949 | AB017020 | 476 | 100 | JKTBP | Mus musculus |
| 949 | BC021374 | 476 | 100 | heterogeneous nuclear | Mus musculus |
| | | | | ribonucleoprotein D-like | |
| 950 | AAH02673 | 1063 | 100 | BC002673 protein kinase H11; | Homo sapiens |
| | | | | small stress protein-like protein | |
| | | | | HSP22 | |
| 950 | AF191017_1 | 1063 | 100 | AF191017 E2IG1 | Homo sapiens |
| 950 | AF250138_1 | 1063 | 100 | AF250138 small stress protein- | Homo sapiens |
| | | | | like protein HSP22 | |
| 951 | AAH05033 | 4718 | 99 | BC005033 Unknown (protein for | Homo sapiens |
| | | | | MGC: 12692) | |
| 951 | AJ289242 | 4654 | 97 | alpha-actinin 4 | Mus musculus |
| 951 | BC013616 | 4654 | 97 | alpha actinin 4 | Mus musculus |
| 952 | AAH11908 | 1787 | 100 | BC011908 Unknown (protein for | Homo sapiens |
| | | | | MGC: 20321) | |
| 952 | AAH14564 | 1787 | 100 | BC014564 Unknown (protein for | Homo sapiens |
| | | | | MGC: 2063) | |
| 952 | AAH18695 | 1787 | 100 | BC018695 Unknown (protein for | Homo sapiens |
| | | | | MGC: 3838) | |
| 953 | AAH12977 | 1363 | 100 | BC012977 nuclear LIM interactor- | Homo sapiens |
| | | | | interacting factor | |
| 953 | AF229162_1 | 1363 | 100 | AF229162 nuclear LIM interactor- | Homo sapiens |
| | | | | interacting factor | |
| 953 | AF229163 | 1363 | 100 | nuclear LIM interactor- | Homo sapiens |
| | | | | interacting factor | |
| 954 | AAH09718 | 557 | 92 | BC009718 epithelial membrane | Homo sapiens |
| | | | | protein 3 | |
| 954 | U52101 | 557 | 92 | YMP | Homo sapiens |
| 954 | U87947 | 557 | 92 | hematopoietic neural membrane | Homo sapiens |
| | | | | protein | |
| 955 | AF162781_1 | 4986 | 94 | AF162781 SH2-containing inositol | Mus musculus |
| | | | | 5-phosphatase 2 | |
| 955 | L36818 | 6044 | 99 | 51C protein | Homo sapiens |
| 955 | Y14385 | 5270 | 98 | inositol polyphosphate 5- | Homo sapiens |
| | | | | phosphatase | |
| 956 | AAH17369 | 1238 | 100 | BC017369 splicing factor, | Homo sapiens |
| | | | | arginine/serine-rich 7 (35 kD) | |
| 956 | AAH17908 | 1238 | 100 | BC017908 splicing factor, | Homo sapiens |
| | | | | arginine/serine-rich 7 (35 kD) | |
| 956 | BC022328 | 1238 | 100 | splicing factor, arginine/serine- | Homo sapiens |
| | | | | rich 7 (35 kD) | |
| 957 | AAH14323 | 1209 | 99 | BC014323 Similar to accessory | Homo sapiens |
| | | | | proteins BAP31/BAP29 | |
| 957 | X81109 | 1213 | 100 | tumor-associated antigen | Homo sapiens |
| 957 | Z31696 | 1213 | 100 | CDM | Homo sapiens |
| 958 | AAH15763 | 3102 | 100 | BC015763 hypothetical protein | Homo sapiens |
| | | | | FLJ20258 | |
| 958 | AK000265 | 3093 | 99 | unnamed protein product | Homo sapiens |
| 958 | AK075098 | 2998 | 99 | unnamed protein product | Homo sapiens |
| 959 | AAH07858 | 2250 | 100 | BC007858 inhibin, beta A (activin | Homo sapiens |
| | | | | A, activin AB alpha polypeptide) | |
| 959 | M13436 | 2250 | 100 | beta-A inhibin | Homo sapiens |
| 959 | X57578 | 2250 | 100 | activin beta-A subunit | Homo sapiens |
| 960 | AK027598 | 3474 | 100 | unnamed protein product | Homo sapiens |
| 960 | AX481758 | 3492 | 99 | unnamed protein product | Homo sapiens |
| 960 | AX481760 | 3492 | 99 | unnamed protein product | Homo sapiens |
| 961 | AAH15958 | 3278 | 100 | BC015958 Unknown (protein for | Homo sapiens |
| | | | | MGC: 15290) | |
| 961 | BC023520 | 3278 | 100 | Unknown (protein for MGC: 12727) | Homo sapiens |
| 961 | U68105 | 3278 | 100 | poly(A)-binding protein | Homo sapiens |
| 962 | AAH10623 | 3729 | 96 | BC010623 Similar to nuclear | Homo sapiens |
| | | | | factor (erythroid-derived 2)-like 1 | |
| 962 | AK090459 | 4032 | 100 | FLJ00380 protein | Homo sapiens |
| 962 | X77366 | 3926 | 100 | hbZ17 | Homo sapiens |
| 963 | AAH03177 | 3500 | 100 | BC003177 KIAA1536 protein | Homo sapiens |
| 963 | AK027881 | 3480 | 99 | unnamed protein product | Homo sapiens |
| 963 | AL136895 | 3500 | 100 | hypothetical protein | Homo sapiens |
| 964 | AY054301 | 4981 | 94 | type III preprocollagen alpha 1 | Homo sapiens |
| | | | | chain | |
| 964 | BC028178 | 6603 | 100 | Similar to collagen, type III, | Homo sapiens |
| | | | | alpha 1 (Ehlers-Danlos syndrome | |
| | | | | type IV, autosomal dominant) | |
| 964 | X14420 | 4981 | 94 | prepro-alpha-1 type 3 collagen | Homo sapiens |
| 965 | AAH11819 | 3473 | 99 | BC011819 DEAD/H (Asp-Glu-Ala- | Homo sapiens |
| | | | | Asp/His) box polypeptide 3 | |
| 965 | AF000982 | 3476 | 100 | dead box, X isoform | Homo sapiens |
| 965 | AF000983 | 3476 | 100 | dead box, X isoform | Homo sapiens |
| 966 | AAH14959 | 1160 | 100 | BC014959 RING1 and YY1 binding | Homo sapiens |
| | | | | protein | |
| 966 | AF179286_1 | 1160 | 100 | AF179286 death effector domain- | Homo sapiens |
| | | | | associated factor | |
| 966 | BC036459 | 1156 | 99 | RING1 and YY1 binding protein | Homo sapiens |
| 967 | AAH17172 | 634 | 99 | BC017172 hypothetical protein | Homo sapiens |
| 967 | AF110774_1 | 634 | 99 | AF110774 adrenal gland protein | Homo sapiens |
| | | | | AD-001 | |
| 967 | AX354355 | 634 | 99 | unnamed protein product | Homo sapiens |
| 968 | AAH08442 | 1106 | 100 | BC008442 Similar to transmembrane | Homo sapiens |
| | | | | 4 superfamily member 1 | |
| 968 | AAH10166 | 1106 | 100 | BC010166 Similar to transmembrane | Homo sapiens |
| | | | | 4 superfamily member 1 | |
| 968 | M90657 | 1106 | 100 | L6 | Homo sapiens |
| 969 | AAH10354 | 1764 | 100 | BC010354 PTD010 protein | Homo sapiens |
| 969 | AB009685 | 1764 | 100 | DERP2 | Homo sapiens |
| 969 | AF131820 | 1764 | 100 | Unknown | Homo sapiens |
| 970 | AF217787 | 1172 | 99 | extracellular glutathione | Homo sapiens |
| | | | | peroxidase | |
| 970 | D00632 | 1195 | 100 | glutathione peroxidase | Homo sapiens |
| 970 | X58295 | 1195 | 100 | glutathione peroxidase | Homo sapiens |
| 971 | AAH00531 | 939 | 97 | BC000531 insulin-like growth | Homo sapiens |
| | | | | factor 2 (somatomedin A) | |
| 971 | AF517226_1 | 939 | 97 | AF517226 insulin-like growth | Homo sapiens |
| | | | | factor 2 (somatomedin A) | |
| 971 | M17863 | 942 | 97 | preproinsulin-like growth factor | Homo sapiens |
| | | | | II, domains A-E | |
| 972 | AJ310544 | 2183 | 100 | EGLN2 protein | Homo sapiens |
| 972 | AX553864 | 2183 | 100 | unnamed protein product | Homo sapiens |
| 972 | BC036051 | 2183 | 100 | Similar to egl nine homolog 2 (C. elegans) | Homo sapiens |
| 973 | AAH00867 | 915 | 98 | BC000867 PRO0659 protein | Homo sapiens |
| 973 | AL834135 | 1118 | 99 | hypothetical protein | Homo sapiens |
| 973 | AX405861 | 1118 | 99 | unnamed protein product | Homo sapiens |
| 974 | AL136890 | 1293 | 98 | hypothetical protein | Homo sapiens |
| 974 | AL137798 | 878 | 80 | dJ1182A14.5.2 (novel gene | Homo sapiens |
| | | | | (isoform 2) KIAA1245 protein) | |
| 974 | BC024011 | 1747 | 100 | Similar to hypothetical protein | Homo sapiens |
| | | | | DKFZp434D177 | |
| 975 | AB037755 | 4896 | 99 | KIAA1334 protein | Homo sapiens |
| 975 | AF155135_1 | 4845 | 99 | AF155135 novel retinal pigment | Homo sapiens |
| | | | | epithelial cell protein | |
| 975 | AF202315_1 | 4141 | 85 | AF202315 ankycorbin | Mus musculus |
| 976 | AK097965 | 344 | 77 | unnamed protein product | Homo sapiens |
| 976 | AX400008 | 283 | 62 | unnamed protein product | Homo sapiens |
| 976 | BC033959 | 342 | 70 | similar to Alu subfamily SQ | Homo sapiens |
| | | | | sequence contamination warning | |
| | | | | entry | |
| 977 | AAH12444 | 1319 | 100 | BC012444 Similar to chloride | Homo sapiens |
| | | | | intracellular channel 4 | |
| 977 | AF097330_1 | 1305 | 99 | AF097330 H1 chloride channel; | Homo sapiens |
| | | | | p64H1; CLIC4 | |
| 977 | AL117424 | 1319 | 100 | hypothetical protein | Homo sapiens |
| 978 | AB034747 | 904 | 100 | small integral membrane protein | Homo sapiens |
| | | | | of lysosome/late endosome | |
| 978 | AF171100 | 812 | 85 | TBX1 protein | Mus musculus |
| 978 | BC018559 | 812 | 85 | LPS-induced TNF-alpha factor | Mus musculus |
| 979 | AAH10645 | 1737 | 100 | BC010645 Similar to | Homo sapiens |
| | | | | sialyltransferase 4C (beta- | |
| | | | | galactosidase alpha-2,3- | |
| | | | | sialytransferase) | |
| 979 | AY040826 | 1715 | 97 | alpha 2,3-sialyltranferase IV | Homo sapiens |
| | | | | type B + 18 | |
| 979 | L23767 | 1710 | 100 | a2,3 sialyltransferse | Homo sapiens |
| 980 | AAH09329 | 887 | 82 | BC009329 triosephosphate | Homo sapiens |
| | | | | isomerase 1 | |
| 980 | AAH11611 | 887 | 82 | BC011611 Similar to | Homo sapiens |
| | | | | triosephosphate isomerase 1 | |
| 980 | AAH15100 | 887 | 82 | BC015100 triosephosphate | Homo sapiens |
| | | | | isomerase 1 | |
| 981 | AAH18986 | 1338 | 98 | BC018986 Unknown (protein for | Homo sapiens |
| | | | | MGC: 20092) | |
| 981 | J05594 | 1331 | 98 | NAD+-dependent 15- | Homo sapiens |
| | | | | hydroxyprostaglandin | |
| | | | | dehydrogenase | |
| 981 | L76465 | 1331 | 98 | NAD+-dependent 15- | Homo sapiens |
| | | | | hydroxyprostaglandin | |
| | | | | dehydrogenase | |
| 982 | AAH00690 | 794 | 100 | BC000690 ribosomal protein L24 | Homo sapiens |
| 982 | BC002110 | 794 | 100 | ribosomal protein L24 | Mus musculus |
| 982 | X78443 | 794 | 100 | ribosomal protein L24 | Rattus |
| | | | | | norvegicus |
| 983 | AAH11402 | 1122 | 100 | BC011402 Similar to decidual | Homo sapiens |
| | | | | protein induced by progesterone | |
| 983 | AB022718 | 1122 | 100 | DEPP | Homo sapiens |
| 983 | AL136653 | 1116 | 99 | hypothetical protein | Homo sapiens |
| 984 | AF108841 | 406 | 74 | pol protein | Homo sapiens |
| 984 | AF108843 | 420 | 75 | pol protein | Homo sapiens |
| 984 | U88902 | 319 | 73 | integrase | Homo sapiens |
| 985 | A07358 | 1657 | 99 | VAC beta | Mus musculus |
| 985 | AAH04376 | 1666 | 100 | BC004376 annexin A8 | Homo sapiens |
| 985 | X16662 | 1657 | 99 | vascular anticoagulant-beta (AA 1-327) | Homo sapiens |
| 986 | AF068227 | 2236 | 100 | putative transmembrane protein | Homo sapiens |
| 986 | AK032293 | 1426 | 76 | unnamed protein product | Mus musculus |
| 986 | BC025487 | 1447 | 77 | Similar to ceroid-lipofuscinosis, | Mus musculus |
| | | | | neuronal 5 | |
| 987 | AAH10652 | 1355 | 100 | BC010652 Unknown (protein for | Homo sapiens |
| | | | | MGC: 9753) | |
| 987 | AF217980_1 | 1338 | 98 | AF217980 unknown | Homo sapiens |
| 988 | AK000496 | 378 | 68 | unnamed protein product | Homo sapiens |
| 988 | AK025116 | 403 | 72 | unnamed protein product | Homo sapiens |
| 988 | AK090511 | 363 | 68 | unnamed protein product | Homo sapiens |
| 989 | AF001900 | 1702 | 100 | secreted frizzled-related protein | Homo sapiens |
| 989 | AF056087 | 1702 | 100 | secreted frizzled related protein | Homo sapiens |
| 989 | BC036503 | 1690 | 99 | Unknown (protein for MGC: 43294) | Homo sapiens |
| 990 | AAH14052 | 2127 | 100 | BC014052 transcriptional coactivator | Homo sapiens |
| | | | | with PDZ-binding motif | |
| | | | | (TAZ) | |
| 990 | AJ299431 | 2127 | 100 | transcriptional coactivator | Homo sapiens |
| 990 | AK022036 | 2127 | 100 | unnamed protein product | Homo sapiens |
| 991 | AAH07493 | 552 | 99 | BC007493 galactosidase, beta 1 | Homo sapiens |
| 991 | M22590 | 552 | 99 | beta-galactosidase precursor (EC | Homo sapiens |
| | | | | 3.2.1.23) | |
| 991 | M34423 | 552 | 99 | beta-galactosidase precursor (EC | Homo sapiens |
| | | | | 3.2.1.23) | |
| 992 | AAH15928 | 2186 | 100 | BC015928 Unknown (protein for | Homo sapiens |
| | | | | MGC: 8773) | |
| 992 | AB037797 | 2313 | 100 | KIAA1376 protein | Homo sapiens |
| 992 | AK088288 | 2153 | 98 | unnamed protein product | Mus musculus |
| 993 | AF087873_1 | 383 | 100 | AF087873 protein kinase inhibitor | Homo sapiens |
| 993 | AF225513_1 | 383 | 100 | AF225513 cAMP-dependent protein | Homo sapiens |
| | | | | kinase inhibitor beta | |
| 993 | BC036011 | 383 | 100 | protein kinase (cAMP-dependent, | Homo sapiens |
| | | | | catalytic) inhibitor beta | |
| 994 | X53305 | 736 | 100 | stathmin | Homo sapiens |
| 994 | X94912 | 736 | 100 | Pr22 | Homo sapiens |
| 994 | Z11566 | 736 | 100 | Pr22 protein | Homo sapiens |
| 995 | AF148856_2 | 150 | 79 | AF148856 unknown | Homo sapiens |
| 995 | AL096744 | 153 | 82 | hypothetical protein | Homo sapiens |
| 995 | M80341 | 150 | 79 | ORF2 | Homo sapiens |
| 996 | AK002154 | 798 | 94 | unnamed protein product | Homo sapiens |
| 996 | AK023417 | 819 | 100 | unnamed protein product | Homo sapiens |
| 996 | AX399949 | 230 | 64 | unnamed protein product | Homo sapiens |
| 997 | AF151807_1 | 2196 | 99 | AF151807 CGI-49 protein | Homo sapiens |
| 997 | AK075178 | 2149 | 97 | unnamed protein product | Homo sapiens |
| 997 | BC026185 | 2136 | 97 | Similar to CGI-49 protein | Homo sapiens |
| 998 | AAH10123 | 2134 | 100 | BC010123 SH3-domain binding | Homo sapiens |
| | | | | protein 5 (BTK-associated) | |
| 998 | AB005047 | 2134 | 100 | SH3 binding protein | Homo sapiens |
| 998 | AB027562 | 2008 | 94 | vascular endothelial cell | Rattus |
| | | | | specific protein 18 | norvegicus |
| 999 | AJ131023 | 5309 | 99 | oxytocinase/insulin-responsive | Homo sapiens |
| | | | | aminopeptidase, variant 1 | |
| 999 | U62768 | 5318 | 99 | oxytocinase splice variant 1 | Homo sapiens |
| 999 | U62769 | 5242 | 99 | oxytocinase splice variant 2 | Homo sapiens |
| 1000 | AF234263 | 2521 | 99 | cathepsin C | Homo sapiens |
| 1000 | U79415 | 2527 | 100 | prepro dipeptidyl peptidase I | Homo sapiens |
| 1000 | X87212 | 2527 | 100 | cathepsin C | Homo sapiens |
| 1001 | BC003426 | 1438 | 94 | Similar to eukaryotic translation | Mus musculus |
| | | | | initiation factor 2B, subunit 1 | |
| | | | | (alpha, 26 kD) | |
| 1001 | U05821 | 1419 | 92 | translation initiation factor | Rattus |
| | | | | eIF-2B alpha-subunit | norvegicus |
| 1001 | X95648 | 1526 | 100 | alfa subunit | Homo sapiens |
| 1002 | AAH14093 | 4073 | 96 | BC014093 Similar to amiloride | Homo sapiens |
| | | | | binding protein 1 (amine oxidase | |
| | | | | (copper-containing)) | |
| 1002 | U11862 | 4060 | 96 | diamine oxidase, copper/topa | Homo sapiens |
| | | | | quinone-containing | |
| 1002 | X78212 | 4066 | 96 | amiloride binding protein | Homo sapiens |
| 1003 | AK003633 | 925 | 98 | unnamed protein product | Mus musculus |
| 1003 | AK007989 | 925 | 98 | unnamed protein product | Mus musculus |
| 1003 | AK076212 | 925 | 98 | unnamed protein product | Mus musculus |
| 1004 | AAH18340 | 1692 | 99 | BC018340 Unknown (protein for | Homo sapiens |
| | | | | MGC: 9372) | |
| 1004 | X79066 | 1692 | 99 | ERF-1 | Homo sapiens |
| 1004 | X99404 | 1684 | 99 | Berg36 | Homo sapiens |
| 1005 | AB055977 | 492 | 93 | I3 protein | Homo sapiens |
| 1005 | AF041430 | 492 | 93 | pRGR2 | Homo sapiens |
| 1005 | AF106966 | 492 | 93 | I3 protein | Homo sapiens |
| 1006 | AAH14438 | 1018 | 100 | BC014438 Unknown (protein for | Homo sapiens |
| | | | | MGC: 22947) | |
| 1006 | AE006639_3 | 1018 | 100 | AE006639 HN1 like | Homo sapiens |
| 1006 | AK023154 | 1018 | 100 | unnamed protein product | Homo sapiens |
| 1007 | J03779 | 3944 | 100 | common acute lymphoblastic | Homo sapiens |
| | | | | leukemia antigen precursor | |
| 1007 | M26628 | 3919 | 99 | enkephalinase | Homo sapiens |
| 1007 | Y00811 | 3919 | 99 | CALLA protein (AA 1-750) | Homo sapiens |
| 1008 | AB035304 | 4092 | 100 | cadherin-5 | Homo sapiens |
| 1008 | U84722 | 4086 | 99 | vascular endothelial cadherin | Homo sapiens |
| 1008 | X79981 | 4092 | 100 | VE-cadherin | Homo sapiens |
| 1009 | AX019983 | 739 | 100 | unnamed protein product | Homo sapiens |
| 1009 | BC026254 | 739 | 100 | insulin-like 4 (placenta) | Homo sapiens |
| 1009 | L34838 | 739 | 100 | EPIL | Homo sapiens |
| 1010 | AF139894_1 | 1813 | 100 | AF139894 RNA-binding protein | Mus musculus |
| | | | | alpha-CP1 | |
| 1010 | AF139895_1 | 1813 | 100 | AF139895 RNA-binding protein | Mus musculus |
| | | | | alpha-CP1 | |
| 1010 | BC004793 | 1813 | 100 | poly (rC)-binding protein 1 | Mus musculus |
| 1011 | U12390 | 366 | 88 | beta-galactosidase alpha peptide | Cloning |
| | | | | | vector |
| | | | | | pSport1 |
| 1011 | U12391 | 355 | 88 | beta-galactosidase alpha peptide | Cloning |
| | | | | | vector |
| | | | | | pSport2 |
| 1011 | Y14836 | 290 | 77 | beta-galactosidase | Phagemid |
| | | | | | cloning |
| | | | | | vector |
| | | | | | pTZ19U |
| 1012 | AF218012_1 | 1751 | 100 | AF218012 unknown | Homo sapiens |
| 1012 | AF218030_1 | 1751 | 100 | AF218030 unknown | Homo sapiens |
| 1012 | AL121586 | 2000 | 88 | dJ47704.1.1 (novel protein | Homo sapiens |
| | | | | similar to otoferlin and | |
| | | | | dysferlin, isoform 1) | |
| 1013 | AAH00352 | 1286 | 100 | BC000352 proteasome (prosome, | Homo sapiens |
| | | | | macropain) activator subunit 1 | |
| | | | | (PA28 alpha) | |
| 1013 | AAH07503 | 1286 | 100 | BC007503 proteasome (prosome, | Homo sapiens |
| | | | | macropain) activator subunit 1 | |
| | | | | (PA28 alpha) | |
| 1013 | AF078829_1 | 1286 | 100 | AF078829 proteasome activator | Homo sapiens |
| | | | | PA28 alpha | |
| 1014 | AAH16155 | 521 | 89 | BC016155 Ras homolog enriched in | Homo sapiens |
| | | | | brain 2 | |
| 1014 | AF493921_1 | 521 | 89 | AF493921 Ras family small GTP | Homo sapiens |
| | | | | binding protein RHEB2 | |
| 1014 | Z29677 | 521 | 89 | Ras-related GTP-binding protein | Homo sapiens |
| 1015 | AAH10423 | 2657 | 100 | BC010423 Ig superfamily receptor | Homo sapiens |
| | | | | LNIR | |
| 1015 | AF160477_1 | 2655 | 99 | AF160477 Ig superfamily receptor | Homo sapiens |
| | | | | LNIR precursor | |
| 1015 | AF426163_1 | 2657 | 100 | AF426163 nectin 4 | Homo sapiens |
| 1016 | AJ295142 | 3578 | 99 | C1q receptor protein | Homo sapiens |
| 1016 | BC028075 | 3577 | 99 | complement component 1, q | Homo sapiens |
| | | | | subcomponent, receptor 1 | |
| 1016 | U94333 | 3587 | 100 | C1qR(p) | Homo sapiens |
| 1017 | AAH14271 | 3392 | 100 | BC014271 endoglin (Osler-Rendu- | Homo sapiens |
| | | | | Weber syndrome 1) | |
| 1017 | J05481 | 3319 | 99 | endoglin precursor | Homo sapiens |
| 1017 | U37439 | 3385 | 99 | endoglin | Homo sapiens |
| 1018 | AAH07411 | 540 | 86 | BC007411 Unknown (protein for | Homo sapiens |
| | | | | MGC: 2554) | |
| 1018 | AF051782 | 536 | 85 | diaphanous 1 | Homo sapiens |
| 1018 | U96963 | 495 | 80 | p140mDia | Mus musculus |
| 1019 | AF151966_1 | 1875 | 66 | AF151966 GTPase activating | Gallus |
| | | | | protein Rap1-GAP | gallus |
| 1019 | BC030891 | 2109 | 95 | Unknown (protein for | Mus musculus |
| | | | | IMAGE: 4485689) | |
| 1019 | M64788 | 3477 | 100 | GTPase activating protein | Homo sapiens |
| 1020 | AX206708 | 1968 | 100 | unnamed protein product | Homo sapiens |
| 1020 | M92934 | 1968 | 100 | connective tissue growth factor | Homo sapiens |
| 1020 | X78947 | 1968 | 100 | connective tissue growth factor | Homo sapiens |
| 1021 | AAH18823 | 849 | 100 | BC018823 Unknown (protein for | Homo sapiens |
| | | | | MGC: 1236) | |
| 1021 | AK007729 | 809 | 97 | unnamed protein product | Mus musculus |
| 1021 | U30826 | 849 | 100 | SRp40-1 | Homo sapiens |
| 1022 | AAH07333 | 2823 | 100 | BC007333 ets variant gene 5 (ets- | Homo sapiens |
| | | | | related molecule) | |
| 1022 | X76184 | 2823 | 100 | ets-related protein | Homo sapiens |
| 1022 | X96381 | 2823 | 100 | erm | Homo sapiens |
| 1023 | AAH02599 | 996 | 99 | BC002599 corticotropin releasing | Homo sapiens |
| | | | | hormone | |
| 1023 | AAH11031 | 996 | 99 | BC011031 Similar to corticotropin | Homo sapiens |
| | | | | releasing hormone | |
| 1023 | V00571 | 996 | 99 | corticotropin releasing factor | Homo sapiens |
| 1024 | AK007731 | 1164 | 94 | unnamed protein product | Mus musculus |
| 1024 | AL110239 | 1234 | 99 | hypothetical protein | Homo sapiens |
| 1024 | AX399865 | 1355 | 100 | unnamed protein product | Homo sapiens |
| 1025 | AAH00732 | 1553 | 100 | BC000732 glioblastoma amplified | Homo sapiens |
| | | | | sequence | |
| 1025 | AAH01837 | 1553 | 100 | BC001837 glioblastoma amplified | Homo sapiens |
| | | | | sequence | |
| 1025 | BC030821 | 1553 | 100 | glioblastoma amplified sequence | Homo sapiens |
| 1026 | AAH01483 | 2103 | 100 | BC001483 Unknown (protein for | Homo sapiens |
| | | | | MGC: 1364) | |
| 1026 | AAH09241 | 2103 | 100 | BC009241 Unknown (protein for | Homo sapiens |
| | | | | MGC: 3556) | |
| 1026 | BC023599 | 2103 | 100 | TRK-fused gene | Homo sapiens |
| 1027 | AAH12806 | 3278 | 100 | BC012806 solute carrier family 5 | Homo sapiens |
| | | | | (sodium-dependent vitamin | |
| | | | | transporter), member 6 | |
| 1027 | AAH15631 | 3278 | 100 | BC015631 solute carrier family 5 | Homo sapiens |
| | | | | (sodium-dependent vitamin | |
| | | | | transporter), member 6 | |
| 1027 | AF116241 | 3272 | 99 | sodium-dependent multivitamin | Homo sapiens |
| | | | | transporter | |
| 1029 | AAH04324 | 744 | 100 | BC004324 ribosomal protein S16 | Homo sapiens |
| 1029 | AAH07977 | 744 | 100 | BC007977 ribosomal protein S16 | Homo sapiens |
| 1029 | X17665 | 744 | 100 | ribosomal protein S16 (AA 1-146) | Rattus |
| | | | | | Rattus |
| 1030 | AB047555 | 624 | 90 | IZP6 | Mus musculus |
| | | | | | musculus |
| 1030 | AK007948 | 624 | 90 | unnamed protein product | Mus musculus |
| 1030 | BC004729 | 624 | 90 | RIKEN cDNA 1810063P04 gene | Mus musculus |
| 1031 | BC003337 | 1957 | 100 | actin, gamma 1 | Mus musculus |
| 1031 | BC021796 | 1957 | 100 | actin, gamma 1 | Mus musculus |
| 1031 | BC023248 | 1957 | 100 | actin, gamma, cytoplasmic | Mus musculus |
| 1032 | AAH11700 | 2048 | 100 | BC011700 glutamate-ammonia ligase | Homo sapiens |
| | | | | (glutamine synthase) | |
| 1032 | AAH11852 | 2048 | 100 | BC011852 glutamate-ammonia ligase | Homo sapiens |
| | | | | (glutamine synthase) | |
| 1032 | AAH18992 | 2048 | 100 | BC018992 Unknown (protein for | Homo sapiens |
| | | | | MGC: 20095) | |
| 1033 | AF234654_1 | 1155 | 100 | AF234654 PLAC1 | Homo sapiens |
| 1033 | AK075086 | 1155 | 100 | unnamed protein product | Homo sapiens |
| 1033 | BC022335 | 1155 | 100 | placenta-specific 1 | Homo sapiens |
| 1034 | AAB67977 | 4108 | 99 | AF019413 complement factor B | Homo sapiens |
| 1034 | AAH04143 | 4121 | 100 | BC004143 B-factor, properdin | Homo sapiens |
| 1034 | AAH07990 | 4121 | 100 | BC007990 B-factor, properdin | Homo sapiens |
| 1035 | AAH17399 | 355 | 98 | BC017399 Similar to RIKEN cDNA | Homo sapiens |
| | | | | 1810037I17 gene | |
| 1035 | AK007721 | 236 | 68 | unnamed protein product | Mus musculus |
| 1035 | BC002135 | 236 | 68 | Unknown (protein for MGC: 7243) | Mus musculus |
| 1036 | AAH13383 | 1701 | 100 | BC013383 calumenin | Homo sapiens |
| 1036 | AF013759 | 1701 | 100 | calumein | Homo sapiens |
| 1036 | U67280 | 1695 | 99 | calumenin | Homo sapiens |
| 1037 | AF069065 | 596 | 97 | inducible cAMP early repressor | Homo sapiens |
| | | | | type 1 | |
| 1037 | AJ311667 | 588 | 95 | induced cAMP early repressor, | Mus musculus |
| | | | | ICER I | |
| 1037 | D14826 | 608 | 100 | hCREM 2alpha-a protein | Homo sapiens |
| 1038 | AAH00452 | 1043 | 100 | BC000452 Similar to thioredoxin | Homo sapiens |
| | | | | peroxidase 1 | |
| 1038 | AAH03022 | 1043 | 100 | BC003022 Similar to thioredoxin | Homo sapiens |
| | | | | peroxidase 1 | |
| 1038 | BC039428 | 1043 | 100 | Similar to peroxiredoxin 2 | Homo sapiens |
| 1039 | AB056107 | 2995 | 100 | zinc finger protein homologous to | Homo sapiens |
| | | | | mouse Zfp91 | |
| 1039 | AB057443 | 2775 | 100 | testis specific ZFP91 | Homo sapiens |
| 1039 | AF310246_1 | 2586 | 99 | AF310246 FKSG11 | Homo sapiens |
| 1040 | AF023476 | 4954 | 100 | meltrin-L precursor | Homo sapiens |
| 1040 | AF023477 | 3855 | 99 | meltrin-S | Homo sapiens |
| 1040 | D50411 | 3939 | 80 | meltrin alpha | Mus musculus |
| 1041 | AF154121_1 | 3126 | 100 | AF154121 sodium-dependent high- | Homo sapiens |
| | | | | affinity dicarboxylate | |
| | | | | transporter | |
| 1041 | AY072810 | 3057 | 98 | energy metabolism-related sodium- | Homo sapiens |
| | | | | dependent high-affinity | |
| | | | | dicarboxylate transporter | |
| 1041 | BC035966 | 3056 | 98 | similar to solute carrier family | Homo sapiens |
| | | | | 13 (sodium-dependent | |
| | | | | dicarboxylate transporter), | |
| | | | | member 3 | |
| 1042 | AF400594 | 687 | 64 | endothelial nitric oxide synthase | Homo sapiens |
| 1042 | AF519768_1 | 687 | 64 | AF519768 nitric oxide synthase 3 | Homo sapiens |
| | | | | (endothelial cell) | |
| 1042 | L26914 | 687 | 64 | nitric oxide synthase | Homo sapiens |
| 1043 | AAH01462 | 1835 | 100 | BC001462 protease, serine, 8 | Homo sapiens |
| | | | | (prostasin) | |
| 1043 | L41351 | 1835 | 100 | prostasin | Homo sapiens |
| 1043 | U33446 | 1835 | 100 | prostasin | Homo sapiens |
| 1044 | AB051442 | 304 | 71 | KIAA1655 protein | Homo sapiens |
| 1044 | AK090913 | 358 | 64 | unnamed protein product | Homo sapiens |
| 1044 | BC024651 | 276 | 76 | similar to PRO2550 | Homo sapiens |
| 1045 | AF346465 | 7291 | 91 | latent transforming growth factor | Mus musculus |
| | | | | beta binding protein 1S | |
| 1045 | AF489528_1 | 7887 | 99 | AF489528 transforming growth | Homo sapiens |
| | | | | factor-beta binding protein-1S | |
| 1045 | M34057 | 7903 | 100 | transforming growth factor-beta 1 | Homo sapiens |
| | | | | binding protein precursor | |
| 1046 | AY004254 | 1199 | 90 | endoplasmic reticulum protein 29 | Rattus |
| | | | | precursor | norvegicus |
| 1046 | X94910 | 1334 | 100 | ERp28 | Homo sapiens |
| 1046 | Y10264 | 1199 | 90 | ERp29 precursor | Rattus |
| | | | | | norvegicus |
| 1047 | AF119917_58 | 250 | 100 | AF119896 PRO2751 | Homo sapiens |
| 1048 | AB020692 | 4158 | 100 | KIAA0885 protein | Homo sapiens |
| 1048 | BC016898 | 4107 | 98 | Unknown (protein for MGC: 19174) | Mus musculus |
| 1048 | X52311 | 4102 | 98 | unr protein (AA 1-798) | Rattus |
| | | | | | norvegicus |
| 1050 | AL049698 | 7034 | 98 | dJ470B24.1.3 (myeloid/lymphoid or | Homo sapiens |
| | | | | mixed-lineage leukemia (trithorax | |
| | | | | (Drosophila) homolog); | |
| | | | | translocated to, 4 (AF-6) | |
| | | | | (isoform 3)) | |
| 1050 | AL049698 | 7050 | 98 | dJ470B24.1.5 (myeloid/lymphoid or | Homo sapiens |
| | | | | mixed-lineage leukemia (trithorax | |
| | | | | (Drosophila) homolog); | |
| | | | | translocated to, 4 (AF-6) | |
| | | | | (isoform 5)) | |
| 1050 | AL161973 | 7363 | 100 | hypothetical protein | Homo sapiens |
| 1051 | AAH21288 | 2134 | 100 | BC021288 Unknown (protein for | Homo sapiens |
| | | | | MGC: 29494) | |
| 1051 | J03263 | 1934 | 98 | membrane glycoprotein | Homo sapiens |
| 1051 | J04182 | 2041 | 96 | lysosomal membrane glycoprotein-1 | Homo sapiens |
| 1052 | AC002400 | 817 | 100 | Acyl carrier protein, | Homo sapiens |
| | | | | Mitochondrial (ACP) (5′partial) | |
| 1052 | AF087660_1 | 788 | 100 | AF087660 NADH: ubiquinone | Homo sapiens |
| | | | | oxidoreductase SDAP subunit | |
| 1052 | AK018717 | 672 | 85 | unnamed protein product | Mus musculus |
| 1053 | AB031550 | 1490 | 95 | pctp-L | Mus musculus |
| 1053 | AF151810_1 | 1853 | 98 | AF151810 CGI-52 protein | Homo sapiens |
| 1053 | Z81134 | 477 | 41 | Hypothetical protein T28D6.7 | Caenorhabditis |
| | | | | | elegans |
| 1055 | AAH09392 | 5306 | 99 | BC009392 Unknown (protein for | Homo sapiens |
| | | | | MGC: 15329) | |
| 1055 | AB011149 | 5318 | 100 | KIAA0577 protein | Homo sapiens |
| 1055 | AP000512 | 5318 | 100 | RNA helicase | Homo sapiens |
| 1056 | AAH08751 | 3757 | 99 | BC008751 calpain 1, (mu/I) large | Homo sapiens |
| | | | | subunit | |
| 1056 | AAH17200 | 3762 | 100 | BC017200 calpain 1, (mu/I) large | Homo sapiens |
| | | | | subunit | |
| 1056 | X04366 | 3762 | 100 | CANP, large subunit (aa 1-714) | Homo sapiens |
| 1057 | AB093679 | 740 | 100 | ribosomal protein L26 | Macaca |
| | | | | | fascicularis |
| 1057 | X69392 | 740 | 100 | ribosomal protein L26 | Homo sapiens |
| 1057 | X80699 | 740 | 100 | L26 | Mus musculus |
| 1058 | AAH06391 | 1974 | 100 | BC006391 inhibin, alpha | Homo sapiens |
| 1058 | M13981 | 1974 | 100 | inhibin A-subunit precursor | Homo sapiens |
| 1058 | X04445 | 1974 | 100 | preproinhibin | Homo sapiens |
| 1059 | AK000385 | 267 | 65 | unnamed protein product | Homo sapiens |
| 1059 | AK092450 | 270 | 58 | unnamed protein product | Homo sapiens |
| 1059 | AK097214 | 354 | 74 | unnamed protein product | Homo sapiens |
| 1060 | AF130117_48 | 357 | 77 | AF130089 PRO2550 | Homo sapiens |
| 1060 | AK000385 | 363 | 75 | unnamed protein product | Homo sapiens |
| 1060 | U22376 | 327 | 76 | alternatively spliced product | Homo sapiens |
| | | | | using exon 13A | |
| 1061 | AF090930_1 | 172 | 51 | AF090930 PRO0478 | Homo sapiens |
| 1061 | AX399919 | 158 | 68 | unnamed protein product | Homo sapiens |
| 1062 | AF063591_1 | 1401 | 99 | AF063591 brain my033 protein | Homo sapiens |
| 1062 | BC022522 | 1411 | 100 | antigen identified by monoclonal | Homo sapiens |
| | | | | antibody MRC OX-2 | |
| 1062 | BC031103 | 1411 | 100 | antigen identified by monoclonal | Homo sapiens |
| | | | | antibody MRC OX-2 | |
| 1063 | AAH00692 | 2582 | 100 | BC000692 Similar to | Homo sapiens |
| | | | | hyaluronoglucosaminidase 2 | |
| 1063 | AC002455 | 2579 | 99 | human PH-20 homolog (LUCA-2) | Homo sapiens |
| 1063 | U09577 | 2579 | 99 | lysosomal hyaluronidase | Homo sapiens |
| 1064 | AAH02700 | 2294 | 100 | BC002700 Similar to keratin 7 | Homo sapiens |
| 1064 | AF509887 | 2294 | 100 | keratin 7 | Homo sapiens |
| 1064 | AF509892 | 2294 | 100 | keratin 7 | Homo sapiens |
| 1065 | AF523361_1 | 1951 | 99 | AF523361 CD34 antigen | Homo sapiens |
| 1065 | M81945 | 1955 | 100 | hematopoietic stem cell antigen | Homo sapiens |
| 1065 | S53910 | 1951 | 99 | CD34 | Homo sapiens |
| 1066 | AB037891 | 493 | 100 | Bc10 | Mus musculus |
| 1066 | AF303656_1 | 493 | 100 | AF303656 bladder cancer-related | Mus musculus |
| | | | | protein | |
| 1066 | BC026935 | 493 | 100 | bladder cancer associated protein | Mus musculus |
| 1067 | AF322909 | 3026 | 100 | transmembrane glycoprotein HGFIN | Homo sapiens |
| 1067 | BC032783 | 3003 | 97 | Similar to glycoprotein | Homo sapiens |
| | | | | (transmembrane) nmb | |
| 1067 | X76534 | 3026 | 100 | NMB | Homo sapiens |
| 1068 | AAH17352 | 3091 | 100 | BC017352 Unknown (protein for | Homo sapiens |
| | | | | MGC: 29525) | |
| 1068 | AF230388_1 | 3085 | 99 | AF230388 tripartite motif protein | Homo sapiens |
| | | | | TRIM29 alpha | |
| 1068 | L24203 | 3085 | 99 | ataxia-telangiectasia group D- | Homo sapiens |
| | | | | associated protein | |
| 1069 | AK003609 | 1032 | 99 | unnamed protein product | Mus musculus |
| 1069 | AL136635 | 1039 | 100 | hypothetical protein | Homo sapiens |
| 1069 | BC016408 | 1032 | 99 | RIKEN cDNA 1110011F09 gene | Mus musculus |
| 1070 | AF073310 | 7103 | 100 | insulin receptor substrate-2 | Homo sapiens |
| 1070 | AF385932_1 | 7014 | 99 | AF385932 insulin receptor | Homo sapiens |
| | | | | substrate 2 insertion mutant | |
| 1070 | AF385933_1 | 7026 | 99 | AF385933 insulin receptor | Homo sapiens |
| | | | | substrate 2 insertion mutant | |
| 1071 | AAH09586 | 2198 | 91 | BC009586 Unknown (protein for | Homo sapiens |
| | | | | MGC: 14649) | |
| 1071 | D26485 | 2198 | 91 | core I protein | Homo sapiens |
| 1071 | L16842 | 2193 | 90 | ubiquinol-cytochrome c reductase | Homo sapiens |
| | | | | core I protein | |
| 1072 | AF062534 | 1919 | 100 | genethonin 1 | Homo sapiens |
| 1072 | AK050416 | 1083 | 62 | unnamed protein product | Mus musculus |
| 1072 | BC022301 | 1919 | 100 | genethonin 1 | Homo sapiens |
| 1073 | AAH08950 | 956 | 100 | BC008950 Unknown (protein for | Homo sapiens |
| | | | | MGC: 3593) | |
| 1073 | AF112202_1 | 956 | 100 | AF112202 prenylated rab acceptor 1 | Homo sapiens |
| 1073 | AJ133534 | 951 | 99 | prenylated Rab acceptor 1 (PRA1) | Homo sapiens |
| 1074 | AAH14257 | 888 | 100 | BC014257 Similar to DKFZp564J157 | Homo sapiens |
| | | | | protein | |
| 1074 | AAH16064 | 888 | 100 | BC016064 DKFZp564J157 protein | Homo sapiens |
| 1074 | AX405840 | 888 | 100 | unnamed protein product | Homo sapiens |
| 1075 | AAH02394 | 2275 | 100 | BC002394 ceroid-lipofuscinosis, | Homo sapiens |
| | | | | neuronal 3, juvenile (Batten, | |
| | | | | Spielmeyer-Vogt disease) | |
| 1075 | AAH04433 | 2275 | 100 | BC004433 ceroid-lipofuscinosis, | Homo sapiens |
| | | | | neuronal 3, juvenile (Batten, | |
| | | | | Spielmeyer-Vogt disease) | |
| 1075 | U32680 | 2275 | 100 | CLN3 protein | Homo sapiens |
| 1076 | AF443072_1 | 20090 | 99 | AF443072 laminin alpha5 chain | Homo sapiens |
| | | | | precursor | |
| 1076 | AL354836 | 20063 | 99 | bA157P1.1.1 (laminin alpha 5) | Homo sapiens |
| 1076 | AX463738 | 20090 | 99 | laminin 5 | Homo sapiens |
| 1077 | BC024366 | 703 | 100 | ribosomal protein L27 | Mus musculus |
| 1077 | X07424 | 703 | 100 | ribosomal protein L27 (AA 1-136) | Rattus |
| | | | | | norvegicus |
| 1077 | X56852 | 703 | 100 | ribosomal protein L27 | Gallus |
| | | | | | gallus |
| 1078 | AAH13131 | 969 | 100 | BC013131 Similar to hypothetical | Homo sapiens |
| | | | | protein FLJ10702 | |
| 1078 | AK001564 | 969 | 100 | unnamed protein product | Homo sapiens |
| 1078 | BC013719 | 969 | 100 | hypothetical protein FLJ10702 | Mus musculus |
| 1079 | AF214737_1 | 5676 | 100 | AF214737 C9orf10a | Homo sapiens |
| 1079 | BC010304 | 3765 | 93 | Unknown (protein for | Mus musculus |
| | | | | IMAGE: 3597827) | |
| 1079 | D80005 | 5676 | 100 | KIAA0183 | Homo sapiens |
| 1080 | AF113127_1 | 1186 | 99 | AF113127 S1R protein | Homo sapiens |
| 1080 | AF151877_1 | 1242 | 99 | AF151877 CGI-119 protein | Homo sapiens |
| 1080 | AF182041_1 | 1186 | 99 | AF182041 z-protein | Homo sapiens |
| 1081 | AAH11616 | 1514 | 100 | BC011616 Similar to cyclin D3 | Homo sapiens |
| 1081 | AF517525_1 | 1511 | 99 | AF517525 cyclin D3 | Homo sapiens |
| 1081 | M92287 | 1514 | 100 | cyclin D3 | Homo sapiens |
| 1082 | AAH11581 | 4280 | 100 | BC011581 Similar to oxysterol | Homo sapiens |
| | | | | binding protein | |
| 1082 | AF185696_1 | 4280 | 100 | AF185696 oxysterol-binding | Homo sapiens |
| | | | | protein 1 | |
| 1082 | AF185705 | 4280 | 100 | oxysterol binding protein 1 | Homo sapiens |
| 1083 | AJ133816 | 1566 | 36 | viral polypeptide | Porcine |
| | | | | | endogenous |
| | | | | | retrovirus |
| 1083 | D10032 | 1645 | 37 | gag-pol precursor polyprotein | Baboon |
| | | | | | endogenous |
| | | | | | virus strain |
| | | | | | M7 |
| 1083 | M26927 | 1571 | 36 | pol polyprotein | Gibbon ape |
| | | | | | leukemia |
| | | | | | virus |
| 1084 | AF012920 | 625 | 100 | GEC-1 | Cavia |
| | | | | | porcellus |
| 1084 | AF312680 | 625 | 100 | GEC-1 | Cavia |
| | | | | | porcellus |
| 1084 | BC004602 | 625 | 100 | RIKEN cDNA 9130422N19 gene | Mus musculus |
| 1085 | AAH14241 | 1733 | 100 | BC014241 Similar to hypothetical | Homo sapiens |
| | | | | protein, MGC: 7035 | |
| 1085 | AB093636 | 1687 | 96 | hypothetical protein | Macaca |
| | | | | | fascicularis |
| 1085 | AK046512 | 1274 | 74 | unnamed protein product | Mus musculus |
| 1086 | AF140240_1 | 749 | 100 | AF140240 transcription factor | Homo sapiens |
| | | | | TBX3 | |
| 1086 | AK075457 | 749 | 100 | unnamed protein product | Homo sapiens |
| 1086 | BC025258 | 749 | 100 | T-box 3 (ulnar mammary syndrome) | Homo sapiens |
| 1087 | AK025047 | 331 | 68 | unnamed protein product | Homo sapiens |
| 1087 | AK025116 | 330 | 67 | unnamed protein product | Homo sapiens |
| 1087 | AK090511 | 365 | 72 | unnamed protein product | Homo sapiens |
| 1088 | AAH01863 | 320 | 100 | BC001863 hypoxia-inducible | Homo sapiens |
| | | | | protein 2 | |
| 1088 | AF144755_1 | 320 | 100 | AF144755 hypoxia-inducible | Homo sapiens |
| | | | | protein 2 | |
| 1088 | AX105363 | 320 | 100 | unnamed protein product | Homo sapiens |
| 1089 | AAH00718 | 1965 | 98 | BC000718 Similar to dynamitin | Homo sapiens |
| | | | | (dynactin complex 50 kD subunit) | |
| 1089 | AAH09468 | 1965 | 98 | BC009468 dynactin 2 (p50) | Homo sapiens |
| 1089 | AAH14083 | 1965 | 98 | BC014083 dynactin 2 (p50) | Homo sapiens |
| 1090 | AB037669 | 2790 | 100 | L-type amino acid transporter 2 | Homo sapiens |
| 1090 | AF171669_1 | 2790 | 100 | AF171669 glycoprotein-associated | Homo sapiens |
| | | | | amino acid transporter LAT2 | |
| 1090 | Y18483 | 2778 | 99 | SLC7A8 protein | Homo sapiens |
| 1091 | AAH16905 | 228 | 62 | BC016905 Similar to hypothetical | Homo sapiens |
| | | | | protein PRO1722 | |
| 1091 | AF118094_21 | 261 | 65 | AF118082 PRO1902 | Homo sapiens |
| 1091 | AK090913 | 202 | 59 | unnamed protein product | Homo sapiens |
| 1092 | AK075392 | 1588 | 99 | unnamed protein product | Homo sapiens |
| 1092 | AX136157 | 1588 | 99 | unnamed protein product | Homo sapiens |
| 1092 | AX179293 | 1591 | 99 | 21676 ADH | Homo sapiens |
| 1093 | AAH13985 | 2426 | 91 | BC013985 PC2 (positive cofactor | Homo sapiens |
| | | | | 2, multiprotein complex) | |
| | | | | glutanine/Q-rich-associated | |
| | | | | protein | |
| 1093 | AK090465 | 2677 | 99 | FLJ00386 protein | Homo sapiens |
| 1093 | BC017110 | 2677 | 99 | Unknown (protein for MGC: 16010) | Homo sapiens |
| 1094 | AAH10514 | 2363 | 100 | BC010514 clusterin (complement | Homo sapiens |
| | | | | lysis inhibitor, SP-40,40, | |
| | | | | sulfated glycoprotein 2, | |
| | | | | testosterone-repressed prostate | |
| | | | | message 2, apolipoprotein J) | |
| 1094 | AAH19588 | 2363 | 100 | BC019588 clusterin (complement | Homo sapiens |
| | | | | lysis inhibitor, SP-40,40, | |
| | | | | sulfated glycoprotein 2, | |
| | | | | testosterone-repressed prostate | |
| | | | | message 2, apolipoprotein J) | |
| 1094 | X14723 | 2363 | 100 | SP-40,40 prepropetide (AA −22 to | Homo sapiens |
| | | | | 427) | |
| 1095 | AAH06786 | 1161 | 100 | BC006786 cellular repressor of | Homo sapiens |
| | | | | E1A-stimulated genes | |
| 1095 | AAH08628 | 1161 | 100 | BC008628 cellular repressor of | Homo sapiens |
| | | | | E1A-stimulated genes | |
| 1095 | AF084523 | 1161 | 100 | cellular repressor of E1A- | Homo sapiens |
| | | | | stimulated genes CREG | |
| 1096 | AX167114 | 8220 | 99 | unnamed protein product | Homo sapiens |
| 1096 | BC036531 | 8206 | 99 | Unknown (protein for MGC: 33668) | Homo sapiens |
| 1096 | Z74615 | 8220 | 99 | prepro-alpha1 (I) collagen | Homo sapiens |
| 1097 | AB025412 | 5671 | 98 | Ten-m3 | Mus musculus |
| 1097 | AF195418_1 | 5676 | 99 | AF195418 ODZ3 | Mus musculus |
| 1097 | AK001336 | 5447 | 100 | unnamed protein product | Homo sapiens |
| 1098 | AAH03550 | 1419 | 100 | BC003550 Similar to CGI-20 | Homo sapiens |
| | | | | protein | |
| 1098 | AF132954_1 | 1577 | 99 | AF132954 CGI-20 protein | Homo sapiens |
| 1098 | AK003551 | 1613 | 95 | unnamed protein product | Mus musculus |
| 1099 | BC022512 | 244 | 96 | platelet/endothelial cell | Homo sapiens |
| | | | | adhesion molecule (CD31 antigen) | |
| 1099 | L34657 | 244 | 96 | platelet endothelial cell | Homo sapiens |
| | | | | adhesion molecule-1 | |
| 1099 | M28526 | 244 | 96 | PECAM-1 precursor | Homo sapiens |
| 1100 | AAH12138 | 1849 | 100 | BC012138 Similar to guanine | Homo sapiens |
| | | | | nucleotide binding protein (G | |
| | | | | protein), alpha inhibiting | |
| | | | | activity polypeptide 2 | |
| 1100 | AF493906_1 | 1849 | 100 | AF493906 guanine nucleotide | Homo sapiens |
| | | | | binding protein alpha i2 | |
| 1100 | X04828 | 1849 | 100 | G protein alpha-subunit (AA 1-355) | Homo sapiens |
| 1101 | AF081258_1 | 3091 | 100 | AF081258 testis-specific | Homo sapiens |
| | | | | chromodomain Y-like protein | |
| 1101 | AF081259_1 | 3091 | 100 | AF081259 testis-specific | Homo sapiens |
| | | | | chromodomain Y-like protein | |
| 1101 | AF081261_1 | 2614 | 88 | AF081261 testis-specific | Mus musculus |
| | | | | chromodomain Y-like protein | |
| 1102 | AF162856_1 | 5147 | 65 | AF162856 receptor-like protein | Mus musculus |
| | | | | tyrosine phosphatase rho-1 | |
| 1102 | X58287 | 7569 | 97 | protein-tyrosine phosphatase | Mus musculus |
| 1102 | X58288 | 7731 | 99 | protein-tyrosine phosphatase | Homo sapiens |
| 1103 | AK097965 | 330 | 74 | unnamed protein product | Homo sapiens |
| 1103 | AX400008 | 319 | 69 | unnamed protein product | Homo sapiens |
| 1103 | BC033959 | 362 | 79 | similar to Alu subfamily SQ | Homo sapiens |
| | | | | sequence contamination warning | |
| | | | | entry | |
| 1104 | AAH18991 | 1272 | 100 | BC018991 hepatoma-derived growth | Homo sapiens |
| | | | | factor (high-mobility group | |
| | | | | protein 1-like) | |
| 1104 | AJ237996 | 1205 | 95 | hepatoma derived growth factor | Bos taurus |
| 1104 | D16431 | 1272 | 100 | hepatoma-derived GF | Homo sapiens |
| 1105 | AAH17197 | 1785 | 99 | BC017197 myeloid cell leukemia | Homo sapiens |
| | | | | sequence 1 (BCL2-related) | |
| 1105 | AF147742_1 | 1785 | 99 | AF147742 myeloid cell | Homo sapiens |
| | | | | differentiation protein | |
| 1105 | AF162677 | 1785 | 99 | myeloid cell leukemia protein 1 | Homo sapiens |
| 1106 | AF274892 | 2520 | 100 | glucose transporter 3 | Homo sapiens |
| 1106 | BC039196 | 2520 | 100 | solute carrier family 2 | Homo sapiens |
| | | | | (facilitated glucose | |
| | | | | transporter), member 3 | |
| 1106 | M20681 | 2520 | 100 | glucose transporter-like protein | Homo sapiens |
| 1107 | AAH00730 | 3448 | 100 | BC000730 calpain 6 | Homo sapiens |
| 1107 | AJ000388 | 3439 | 99 | calpain-like protease, CANPX | Homo sapiens |
| 1107 | AL031117 | 3448 | 100 | dJ914P14.1 (calpain-like protease | Homo sapiens |
| | | | | CANPX) | |
| 1108 | AAH03143 | 1980 | 100 | BC003143 dual specificity | Homo sapiens |
| | | | | phosphatase 6 | |
| 1108 | AAH03562 | 1980 | 100 | BC003562 dual specificity | Homo sapiens |
| | | | | phosphatase 6 | |
| 1108 | X93920 | 1980 | 100 | protein-tyrosine-phosphatase | Homo sapiens |
| 1109 | AB023194 | 6006 | 100 | KIAA0977 protein | Homo sapiens |
| 1109 | AK052533 | 3549 | 61 | unnamed protein product | Mus musculus |
| 1109 | AL049939 | 1823 | 99 | hypothetical protein | Homo sapiens |
| 1110 | AAH15031 | 4447 | 100 | BC015031 Unknown (protein for | Homo sapiens |
| | | | | MGC: 9105) | |
| 1110 | AB018266 | 4447 | 100 | KIAA0723 protein | Homo sapiens |
| 1110 | BC029070 | 4375 | 98 | Similar to matrin 3 | Mus musculus |
| 1111 | AAH18726 | 1229 | 100 | BC018726 CD74 antigen (invariant | Homo sapiens |
| | | | | polypeptide of major | |
| | | | | histocompatibility complex, class | |
| | | | | II antigen-associated) | |
| 1111 | M13560 | 1229 | 100 | cell surface glycoprotein | Homo sapiens |
| 1111 | X00497 | 1229 | 100 | putative p33 | Homo sapiens |
| 1112 | AF114262_3 | 2305 | 88 | AF114260 forkhead protein AFXH | Mus musculus |
| 1112 | X93996 | 2624 | 100 | AFX | Homo sapiens |
| 1112 | Y11284 | 2489 | 97 | AFX1 | Homo sapiens |
| 1113 | AAH09409 | 1688 | 100 | BC009409 Unknown (protein for | Homo sapiens |
| | | | | MGC: 10655) | |
| 1113 | X13425 | 1688 | 100 | GA733-1 protein (AA 1-323) | Homo sapiens |
| 1113 | X77754 | 1688 | 100 | gp50/TROP-2 | Homo sapiens |
| 1114 | AAH02532 | 795 | 100 | BC002532 epididymal secretory | Homo sapiens |
| | | | | protein (19.5 kD) | |
| 1114 | X67698 | 795 | 100 | orf | Homo sapiens |
| 1114 | X78134 | 795 | 100 | epididymal secretory protein 14.6 | Macaca |
| | | | | | fascicularis |
| 1115 | AK075092 | 233 | 100 | unnamed protein product | Homo sapiens |
| 1116 | AAH16692 | 1163 | 100 | BC016692 progesterone receptor | Homo sapiens |
| | | | | membrane component 2 | |
| 1116 | AJ002030 | 1163 | 100 | progresterone binding protein | Homo sapiens |
| 1116 | AY069921 | 525 | 60 | membrane progesterone receptor- | Oncorhynchus |
| | | | | like protein | mykiss |
| 1117 | AAH16041 | 1441 | 100 | BC016041 insulin-like growth | Homo sapiens |
| | | | | factor-binding protein 4 | |
| 1117 | M62403 | 1441 | 100 | IGF-BP 4 | Homo sapiens |
| 1117 | Y12508 | 1441 | 100 | insulin-like growth factor | Homo sapiens |
| | | | | binding protein 4 | |
| 1118 | AK000575 | 734 | 100 | unnamed protein product | Homo sapiens |
| 1118 | AK013438 | 484 | 72 | unnamed protein product | Mus musculus |
| 1118 | AY116892 | 734 | 100 | HCF-1 beta-propeller interacting | Homo sapiens |
| | | | | protein | |
| 1119 | AAH14410 | 2710 | 100 | BC014410 Similar to EGF- | Homo sapiens |
| | | | | containing fibulin-like | |
| | | | | extracellular matrix protein 1 | |
| 1119 | AK077302 | 2581 | 94 | unnamed protein product | Mus musculus |
| 1119 | AY004330 | 2710 | 100 | EGF-containing fibulin-like | Homo sapiens |
| | | | | extracellular matrix protein 1 | |
| 1120 | AF102552 | 11066 | 84 | 270 kDa ankyrin G isoform | Rattus |
| | | | | | norvegicus |
| 1120 | L40632 | 7184 | 95 | ankyrin 3 | Mus musculus |
| 1120 | U13616 | 20107 | 99 | ankyrin G | Homo sapiens |
| 1121 | AC002073 | 3406 | 100 | Lim Kinase | Homo sapiens |
| 1121 | AL117466 | 3183 | 100 | hypothetical protein | Homo sapiens |
| 1121 | D45906 | 3406 | 100 | LIMK-2 | Homo sapiens |
| 1122 | AAH00836 | 633 | 100 | BC000836 CGI-127 protein | Homo sapiens |
| 1122 | AF135161_1 | 633 | 100 | AF135161 unknown | Homo sapiens |
| 1122 | AF151885_1 | 633 | 100 | AF151885 CGI-127 protein | Homo sapiens |
| 1123 | A16768 | 356 | 79 | kunitz type protease inhibitor | synthetic |
| | | | | | construct |
| 1123 | BC033174 | 501 | 98 | Similar to collagen, type VI, | Homo sapiens |
| | | | | alpha 3 | |
| 1123 | X52022 | 501 | 98 | collagen type VI, alpha 3 chain | Homo sapiens |
| 1124 | AAH04440 | 776 | 100 | BC004440 Unknown (protein for | Homo sapiens |
| | | | | MGC: 4033) | |
| 1124 | AAH19336 | 821 | 100 | BC019336 Unknown (protein for | Homo sapiens |
| | | | | IMAGE: 3617778) | |
| 1124 | U91512 | 770 | 99 | ninjurin | Homo sapiens |
| 1125 | AAH18127 | 1250 | 93 | BC018127 Similar to | Homo sapiens |
| | | | | Peptidylglycine alpha-amidating | |
| | | | | monooxygenase | |
| 1125 | AB095007 | 1250 | 93 | peptidylglycine alpha-amidating | Homo sapiens |
| | | | | monooxygenase | |
| 1125 | AF035320 | 1279 | 100 | peptidylglycine alpha-amidating | Homo sapiens |
| | | | | monooxygenase | |
| 1126 | AAH13398 | 6167 | 100 | BC013398 pumilio (Drosophila) | Homo sapiens |
| 1126 | AF315592_1 | 6167 | 100 | AF315592 Pumilio 1 | Homo sapiens |
| 1126 | D43951 | 6233 | 100 | KIAA0099 protein | Homo sapiens |
| 1127 | AAH00465 | 826 | 100 | BC000465 growth arrest and DNA- | Homo sapiens |
| | | | | damage-inducible, gamma | |
| 1127 | AAH19325 | 826 | 100 | BC019325 growth arrest and DNA- | Homo sapiens |
| | | | | damage-inducible, gamma | |
| 1127 | AF494037_1 | 826 | 100 | AF494037 growth arrest and DNA- | Homo sapiens |
| | | | | damage-inducible, gamma | |
| 1128 | AAH01955 | 883 | 100 | BC001955 ribosomal protein S10 | Homo sapiens |
| 1128 | AAH04334 | 883 | 100 | BC004334 ribosomal protein S10 | Homo sapiens |
| 1128 | AAH05012 | 883 | 100 | BC005012 ribosomal protein S10 | Homo sapiens |
| 1129 | AAH12740 | 4687 | 99 | BC012740 Similar to dystroglycan | Homo sapiens |
| | | | | 1 (dystrophin-associated | |
| | | | | glycoprotein 1) | |
| 1129 | AAH14616 | 4687 | 99 | BC014616 Unknown (protein for | Homo sapiens |
| | | | | MGC: 3618) | |
| 1129 | L19711 | 4703 | 100 | dystroglycan | Homo sapiens |
| 1130 | AAH20983 | 978 | 100 | BC020983 pituitary tumor- | Homo sapiens |
| | | | | transforming 1 interacting | |
| | | | | protein | |
| 1130 | BC031097 | 978 | 100 | pituitary tumor-transforming 1 | Homo sapiens |
| | | | | interacting protein | |
| 1130 | BC034250 | 978 | 100 | pituitary tumor-transforming 1 | Homo sapiens |
| | | | | interacting protein | |
| 1131 | AAH07082 | 185 | 100 | BC007082 SWI/SNF related, matrix | Homo sapiens |
| | | | | associated, actin dependent | |
| | | | | regulator of chromatin, subfamily | |
| | | | | e, member 1 | |
| 1131 | AAH11017 | 185 | 100 | BC011017 SWI/SNF related, matrix | Homo sapiens |
| | | | | associated, actin dependent | |
| | | | | regulator of chromatin, subfamily | |
| | | | | e, member 1 | |
| 1131 | AF035262 | 185 | 100 | BAF57 | Homo sapiens |
| 1133 | AF099154 | 13656 | 86 | von Willebrand factor | Canis |
| | | | | | familiaris |
| 1133 | M25865 | 15510 | 99 | von Willebrand factor | Homo sapiens |
| 1133 | X04385 | 15515 | 100 | VWF pre-pro-polypeptide (−22 to | Homo sapiens |
| | | | | 2791) | |
| 1134 | AAH17692 | 3143 | 99 | BC017692 Similar to quiescin Q6 | Homo sapiens |
| 1134 | AF361868 | 3135 | 98 | sulfhydryl oxidase | Homo sapiens |
| 1134 | U97276 | 3911 | 99 | quiescin | Homo sapiens |
| 1136 | AAH12123 | 1528 | 100 | BC012123 golgi phosphoprotein 3 | Homo sapiens |
| 1136 | AJ296152 | 1528 | 100 | Golgi protein | Homo sapiens |
| 1136 | BC033725 | 1528 | 100 | golgi phosphoprotein 3 (coat- | Homo sapiens |
| | | | | protein) | |
| 1137 | AAH08726 | 2687 | 100 | BC008726 oxidative-stress | Homo sapiens |
| | | | | responsive 1 | |
| 1137 | AB017642 | 2687 | 100 | oxidative-stress responsive 1 | Homo sapiens |
| 1137 | AB029024 | 2355 | 100 | KIAA1101 protein | Homo sapiens |
| 1138 | AAH00478 | 3403 | 99 | BC000478 heat shock 70 kD protein | Homo sapiens |
| | | | | 9B (mortalin-2) | |
| 1138 | BC024034 | 3403 | 99 | heat shock 70 kD protein 9B | Homo sapiens |
| | | | | (mortalin-2) | |
| 1138 | BC030634 | 3399 | 99 | heat shock 70 kD protein 9B | Homo sapiens |
| | | | | (mortalin-2) | |
| 1139 | BC006834 | 892 | 100 | B-cell translocation gene 1, | Mus musculus |
| | | | | anti-proliferative | |
| 1139 | BC018309 | 892 | 100 | B-cell translocation gene 1, | Mus musculus |
| | | | | anti-proliferative | |
| 1139 | Z16410 | 892 | 100 | btg1 | Mus musculus |
| 1140 | AK075720 | 517 | 92 | unnamed protein product | Mus musculus |
| 1140 | BC003862 | 517 | 92 | Similar to transmembrane 9 | Mus musculus |
| | | | | superfamily member 2 | |
| 1140 | U81006 | 518 | 93 | p76 | Homo sapiens |
| 1141 | A03736 | 3879 | 100 | factor XIIIa | Homo sapiens |
| 1141 | M14354 | 3879 | 100 | clotting factor XIIIa precursor | Homo sapiens |
| | | | | (EC 2.3.2.13) | |
| 1141 | M14539 | 4016 | 99 | factor XIII precursor | Homo sapiens |
| 1142 | AAH06541 | 969 | 99 | BC006541 integrin, beta 5 | Homo sapiens |
| 1142 | J05633 | 962 | 98 | integrin beta-5 subunit precursor | Homo sapiens |
| 1142 | X53002 | 969 | 99 | precusor polypeptide (AA −23 to | Homo sapiens |
| | | | | 776) | |
| 1143 | D50929 | 7262 | 100 | The KIAA0139 gene product is | Homo sapiens |
| | | | | related to mouse centrosomin B. | |
| 1143 | U58046 | 7262 | 100 | p167 | Homo sapiens |
| 1143 | U78311 | 7262 | 100 | translation initiation factor 3 | Homo sapiens |
| | | | | large subunit | |
| 1144 | AAH01571 | 4708 | 99 | BC001571 eukaryotic translation | Homo sapiens |
| | | | | initiation factor 3, subunit 8 | |
| | | | | (110 kD) | |
| 1144 | AC002544 | 4708 | 99 | Translation initiation factor | Homo sapiens |
| | | | | eIF-p110 | |
| 1144 | U46025 | 4708 | 99 | translation intiation factor eIF-3 | Homo sapiens |
| | | | | p110 subunit | |
| 1145 | AB019987 | 6513 | 99 | chromosome-associated | Homo sapiens |
| | | | | polypeptide-C | |
| 1145 | AF092564 | 6041 | 99 | chromosome-associated protein-C | Homo sapiens |
| 1145 | AL136877 | 6517 | 100 | hypothetical protein | Homo sapiens |
| 1146 | AAH01756 | 2880 | 99 | BC001756 phosphoglucomutase 1 | Homo sapiens |
| 1146 | AAH19920 | 2873 | 99 | BC019920 Unknown (protein for | Homo sapiens |
| | | | | MGC: 29909) | |
| 1146 | M83088 | 2880 | 99 | PGM1 | Homo sapiens |
| 1147 | AB020664 | 3759 | 100 | KIAA0857 protein | Homo sapiens |
| 1147 | AF334812_1 | 3333 | 100 | AF334812 Rab11 interacting | Homo sapiens |
| | | | | protein Rip11a | |
| 1147 | BC035013 | 3303 | 99 | Similar to Rab coupling protein | Homo sapiens |
| 1148 | AF356518_1 | 1637 | 100 | AF356518 junctional adhesion | Homo sapiens |
| | | | | molecule 3 precursor | |
| 1148 | AF448478_1 | 1637 | 100 | AF448478 junctional adhesional | Homo sapiens |
| | | | | molecule-3 | |
| 1148 | AJ416101 | 1870 | 100 | junction adhesion molecule 3 | Homo sapiens |
| 1149 | AF130117_48 | 355 | 77 | AF130089 PRO2550 | Homo sapiens |
| 1149 | AK000385 | 353 | 74 | unnamed protein product | Homo sapiens |
| 1149 | AK097266 | 324 | 79 | unnamed protein product | Homo sapiens |
| 1150 | AAH01562 | 3284 | 100 | BC001562 nuclear receptor | Homo sapiens |
| | | | | coactivator 4 | |
| 1150 | AL162047 | 3284 | 100 | hypothetical protein | Homo sapiens |
| 1150 | L49399 | 3284 | 100 | ORF | Homo sapiens |
| 1151 | AAH02356 | 2379 | 100 | B0002356 nucleobindin 1 | Homo sapiens |
| 1151 | M96824 | 2326 | 98 | nucleobindin | Homo sapiens |
| 1151 | U31342 | 2357 | 99 | nucleobindin | Homo sapiens |
| 1152 | AAH17180 | 4694 | 100 | BC017180 Unknown (protein for | Homo sapiens |
| | | | | MGC: 790) | |
| 1152 | AK088028 | 4597 | 97 | unnamed protein product | Mus musculus |
| 1152 | BC007126 | 4597 | 97 | Unknown (protein for MGC: 5677) | Mus musculus |
| 1153 | AAH08099 | 378 | 100 | BC008099 Unknown (protein for | Homo sapiens |
| | | | | IMAGE: 4183312) | |
| 1154 | AF137053_1 | 2310 | 99 | AF137053 mutant desmin | Homo sapiens |
| 1154 | BC032116 | 2313 | 99 | desmin | Homo sapiens |
| 1154 | U59167 | 2325 | 100 | desmin | Homo sapiens |
| 1155 | AL445795 | 23376 | 99 | heparan sulfate proteoglycan | Homo sapiens |
| | | | | perlecan | |
| 1155 | M85289 | 23477 | 99 | heparan sulfate proteoglycan | Homo sapiens |
| 1155 | X62515 | 23627 | 99 | Human basement membrane heparan | Homo sapiens |
| | | | | sulfate proteoglycan core protein | |
| 1156 | AK057830 | 270 | 51 | unnamed protein product | Homo sapiens |
| 1156 | BC024651 | 210 | 58 | similar to PRO2550 | Homo sapiens |
| 1156 | BC032019 | 230 | 55 | similar to Alu subfamily SQ | Homo sapiens |
| | | | | sequence contamination warning | |
| | | | | entry | |
| 1157 | AAH00429 | 309 | 100 | BC000429 chromosome 14 open | Homo sapiens |
| | | | | reading frame 2 | |
| 1157 | AAH01944 | 309 | 100 | BC001944 chromosome 14 open | Homo sapiens |
| | | | | reading frame 2 | |
| 1157 | AF054175 | 309 | 100 | mitochondrial proteolipid 68MP | Homo sapiens |
| | | | | homolog | |
| 1158 | AAH01263 | 2152 | 100 | BC001263 serum/glucocorticoid | Homo sapiens |
| | | | | regulated kinase | |
| 1158 | AF153609_1 | 2152 | 100 | AF153609 serine/threonine protein | Homo sapiens |
| | | | | kinase sgk | |
| 1158 | AX553549 | 2152 | 100 | unnamed protein product | Homo sapiens |
| 1159 | AAH07103 | 710 | 100 | BC007103 small acidic protein | Homo sapiens |
| 1159 | AAH16352 | 710 | 100 | BC016352 small acidic protein | Homo sapiens |
| 1159 | AAH20937 | 710 | 100 | BC020937 small acidic protein | Homo sapiens |
| 1161 | AY043487 | 412 | 100 | selenoprotein SelM | Homo sapiens |
| 1161 | AY043488 | 367 | 85 | selenoprotein SelM | Mus musculus |
| 1161 | BC019742 | 345 | 85 | Unknown (protein for MGC: 30803) | Mus musculus |
| 1162 | AF124719_1 | 1018 | 100 | AF124719 GM2 activator protein | Homo sapiens |
| 1162 | X61095 | 1039 | 97 | GM2-activator protein | Homo sapiens |
| 1162 | X62078 | 1018 | 100 | GM2 activator protein | Homo sapiens |
| 1163 | AB028974 | 1802 | 100 | KIAA1051 protein | Homo sapiens |
| 1163 | AB049150 | 1703 | 100 | MEF3 like 1 | Homo sapiens |
| 1163 | AX082622 | 2129 | 100 | unnamed protein product | Homo sapiens |
| 1164 | BC026690 | 3964 | 100 | CD97 antigen | Homo sapiens |
| 1164 | U76764 | 3796 | 87 | CD97 | Homo sapiens |
| 1164 | X84700 | 3951 | 99 | leucocyte antigen CD97 | Homo sapiens |
| 1165 | K03195 | 2485 | 99 | glucose transporter glycoprotein | Homo sapiens |
| 1165 | M13979 | 2430 | 97 | glucose-transporter protein | Rattus |
| | | | | | norvegicus |
| 1165 | M22063 | 2430 | 97 | glucose transporter protein | Rattus |
| | | | | | norvegicus |
| 1166 | AAH19297 | 1801 | 100 | BC019297 Unknown (protein for | Homo sapiens |
| | | | | MGC: 4111) | |
| 1166 | AK021655 | 1801 | 100 | unnamed protein product | Homo sapiens |
| 1166 | AK022757 | 1801 | 100 | unnamed protein product | Homo sapiens |
| 1167 | AC005943 | 1522 | 100 | methyl-CpG binding protein MBD3 | Homo sapiens |
| 1167 | AF072247 | 1522 | 100 | methyl-CpG binding domain- | Homo sapiens |
| | | | | containing protein MBD3 | |
| 1167 | AF072248 | 1427 | 95 | methyl-CpG binding domain- | Mus musculus |
| | | | | containing protein MBD3 | |
| 1168 | AB018009 | 2602 | 100 | L-type amino acid transporter 1 | Homo sapiens |
| 1168 | AB018542 | 2602 | 100 | CD98 light chain | Homo sapiens |
| 1168 | AF104032 | 2602 | 100 | L-type amino acid transporter | Homo sapiens |
| | | | | subunit LAT1 | |
| 1170 | AAH10737 | 1911 | 100 | BC010737 Similar to reticulon 4 | Homo sapiens |
| 1170 | AAH12619 | 1911 | 100 | BC012619 Unknown (protein for | Homo sapiens |
| | | | | MGC: 13655) | |
| 1170 | BC026788 | 1911 | 100 | reticulon 4 | Homo sapiens |
| 1171 | AAH02412 | 3766 | 99 | BC002412 inner membrane protein, | Homo sapiens |
| | | | | mitochondrial (mitofilin) | |
| 1171 | D21094 | 3766 | 99 | motor protein | Homo sapiens |
| 1171 | L42572 | 3771 | 100 | transmembrane protein | Homo sapiens |
| 1172 | AAH01925 | 573 | 100 | BC001925 FK506-binding protein 1A | Homo sapiens |
| | | | | (12 kD) | |
| 1172 | AAH05147 | 573 | 100 | BC005147 FK506-binding protein 1A | Homo sapiens |
| | | | | (12 kD) | |
| 1172 | M93060 | 573 | 100 | FK506-binding protein 12 | Homo sapiens |
| 1173 | AF116721_20 | 391 | 100 | AF116620 PRO1068 | Homo sapiens |
| 1174 | AK075023 | 1335 | 99 | unnamed protein product | Homo sapiens |
| 1174 | AL117442 | 1344 | 100 | hypothetical protein | Homo sapiens |
| 1174 | BC002138 | 1302 | 96 | Unknown (protein for | Mus musculus |
| | | | | IMAGE: 3484538) | |
| 1175 | AAH07674 | 382 | 100 | BC007674 CD24 antigen (small cell | Homo sapiens |
| | | | | lung carcinoma cluster 4 antigen) | |
| 1175 | L33930 | 382 | 100 | signal transducer CD24 | Homo sapiens |
| 1175 | M58664 | 382 | 100 | signal transducer CD24 | Homo sapiens |
| 1176 | AAH12181 | 4260 | 100 | BC012181 Similar to paired basic | Homo sapiens |
| | | | | amino acid cleaving enzyme | |
| | | | | (furin, membrane associated | |
| | | | | receptor protein) | |
| 1176 | U20436 | 4060 | 95 | furin endoprotease | Cricetulus |
| | | | | | griseus |
| 1176 | X17094 | 4260 | 100 | furin (AA 1-794) | Homo sapiens |
| 1177 | AAH15236 | 1189 | 100 | BC015236 hypothetical protein | Homo sapiens |
| 1177 | AF335324_1 | 1189 | 100 | AF335324 RTP801 | Homo sapiens |
| 1177 | AY090097 | 1189 | 100 | REDD-1 | Homo sapiens |
| 1178 | AK026800 | 289 | 72 | unnamed protein product | Homo sapiens |
| 1178 | AK057830 | 364 | 69 | unnamed protein product | Homo sapiens |
| 1178 | BC032019 | 330 | 70 | similar to Alu subfamily SQ | Homo sapiens |
| | | | | sequence contamination warning | |
| | | | | entry | |
| 1179 | AAH10058 | 3813 | 100 | BC010058 exostoses (multiple) 2 | Homo sapiens |
| 1179 | U62740 | 3813 | 100 | EXT2 | Homo sapiens |
| 1179 | U67368 | 3813 | 100 | multiple exostosis 2 | Homo sapiens |
| 1180 | AAH08301 | 1967 | 100 | BC008301 tuftelin 1 | Homo sapiens |
| 1180 | AF254260_1 | 1967 | 100 | AF254260 tuftelin 1 | Homo sapiens |
| 1180 | AF254860 | 1967 | 100 | tuftelin 1 | Homo sapiens |
| 1182 | AF272662_1 | 9764 | 94 | AF272662 alpha 1 type V collagen | Rattus |
| | | | | | norvegicus |
| 1182 | D90279 | 10311 | 99 | collagen alpha 1(V) chain | Homo sapiens |
| | | | | precursor | |
| 1182 | M76729 | 10277 | 99 | pro-alpha-1 type V collagen | Homo sapiens |
| 1183 | AAH03155 | 1475 | 90 | BC003155 coatomer protein | Homo sapiens |
| | | | | complex, subunit epsilon | |
| 1183 | AAH07250 | 1475 | 90 | BC007250 coatomer protein | Homo sapiens |
| | | | | complex, subunit epsilon | |
| 1183 | AAH17285 | 1475 | 90 | BC017285 Unknown (protein for | Homo sapiens |
| | | | | MGC: 29652) | |
| 1184 | AX354381 | 347 | 100 | unnamed protein product | Homo sapiens |
| 1185 | AF202636_1 | 2164 | 100 | AF202636 angiopoietin-like | Homo sapiens |
| | | | | protein PP1158 | |
| 1185 | AX079971 | 2164 | 100 | unnamed protein product | Homo sapiens |
| 1185 | BC023647 | 2164 | 100 | angiopoietin-like 4 | Homo sapiens |
| 1186 | AK056767 | 1189 | 100 | unnamed protein product | Homo sapiens |
| 1186 | AL831913 | 713 | 85 | hypothetical protein | Homo sapiens |
| 1187 | AAH01270 | 1896 | 100 | BC001270 SM3-domain GRB2-like 1 | Homo sapiens |
| 1187 | U65999 | 1896 | 100 | SH3-containing protein EEN | Homo sapiens |
| 1187 | X99656 | 1896 | 100 | SH3-containing Grb-2-like 1 | Homo sapiens |
| 1188 | AAH00385 | 4505 | 100 | BC000385 Unknown (protein for | Homo sapiens |
| | | | | MGC: 8429) | |
| 1188 | AF102803 | 4505 | 100 | alphaE-catenin | Homo sapiens |
| 1188 | D13866 | 4505 | 100 | alpha-catenin | Homo sapiens |
| 1189 | AAH07564 | 607 | 90 | BC007564 annexin A11 | Homo sapiens |
| 1189 | AJ278464 | 607 | 90 | annexin A11 | Homo sapiens |
| 1189 | AJ278465 | 607 | 90 | annexin A11 | Homo sapiens |
| 1190 | AAH01491 | 1470 | 99 | BC001491 heme oxygenase | Homo sapiens |
| | | | | (decycling) 1 | |
| 1190 | X06985 | 1475 | 100 | heme oxygenase (AA 1-288) | Homo sapiens |
| 1190 | Z82244 | 1475 | 100 | bK286B10.2 (Heme Oxygenase | Homo sapiens |
| | | | | (decycling) 1 (HO-1 EC | |
| | | | | 1.14.99.3)) | |
| 1192 | AAH13910 | 1671 | 100 | BC013910 Similar to death | Homo sapiens |
| | | | | effector domain-containing | |
| 1192 | AAH16724 | 1671 | 100 | BC016724 death effector domain- | Homo sapiens |
| | | | | containing | |
| 1192 | AJ010973 | 1671 | 100 | DEDD protein | Homo sapiens |
| 1193 | AAH17882 | 1276 | 100 | BC017882 proteasome (prosome, | Homo sapiens |
| | | | | macropain) subunit, alpha type 6 | |
| 1193 | BC022354 | 1276 | 100 | proteasome (prosome, macropain) | Homo sapiens |
| | | | | subunit, alpha type 6 | |
| 1193 | D10755 | 1276 | 100 | proteasome subunit R-IOTA | Rattus sp. |
| 1194 | AAH09581 | 2041 | 100 | BC009581 hydroxysteroid (17-beta) | Homo sapiens |
| | | | | dehydrogenase 2 | |
| 1194 | L11708 | 2041 | 100 | 17 beta hydroxysteroid | Homo sapiens |
| | | | | dehydrogenase type 2 | |
| 1194 | L40802 | 2041 | 100 | 17-hydroxysteroid dehydrogenase | Homo sapiens |
| 1195 | AAH08452 | 586 | 100 | BC008452 ATPase, H+ transporting, | Homo sapiens |
| | | | | lysosomal (vacuolar proton pump), | |
| | | | | member J | |
| 1195 | AP038954 | 586 | 100 | vacuolar H(+)-ATPase subunit | Homo sapiens |
| 1195 | S82464 | 573 | 96 | vacuolar H(+)-ATPase subunit; V- | Bos taurus |
| | | | | ATPase subunit; M16 | |
| 1196 | AE003452 | 193 | 35 | CG9350-PB | Drosophila |
| | | | | | melanogaster |
| 1196 | AJ510148 | 571 | 72 | mitochondrial NADH: ubiquinone | Bos taurus |
| | | | | oxidoreductase B14.7 subunit | |
| 1196 | AK008201 | 532 | 65 | unnamed protein product | Mus musculus |
| 1197 | AB020685 | 3216 | 100 | KIAA0878 protein | Homo sapiens |
| 1197 | AK006650 | 2065 | 93 | unnamed protein product | Mus musculus |
| 1197 | BC041337 | 3212 | 99 | Rho-related BTB domain containing 3 | Homo sapiens |
| 1198 | AB058749 | 1868 | 100 | KIAA1846 protein | Homo sapiens |
| 1198 | AK092295 | 1868 | 100 | unnamed protein product | Homo sapiens |
| 1198 | AL035661 | 1868 | 100 | dJ568C11.3 (novel AMP-binding | Homo sapiens |
| | | | | enzyme similar to acetyl-coenzyme | |
| | | | | A synthethase (acetate-coA | |
| | | | | ligase)) | |
| 1199 | AF009242 | 1163 | 100 | proline-rich Gla protein 1 | Homo sapiens |
| 1199 | AF419154 | 572 | 52 | mitotic phosphoprotein 77 | Xenopus |
| | | | | | laevis |
| 1199 | BC030786 | 1246 | 100 | proline-rich Gla (G- | Homo sapiens |
| | | | | carboxyglutamic acid) polypeptide 1 | |
| 1200 | AF000652 | 1503 | 99 | syntenin | Homo sapiens |
| 1200 | AF006636 | 1508 | 100 | melanoma differentiation | Homo sapiens |
| | | | | associated protein-9 | |
| 1200 | U83463 | 1503 | 99 | scaffold protein Pbp1 | Homo sapiens |
| 1201 | AF312032 | 5200 | 99 | ephrin type-B receptor 4 | Homo sapiens |
| | | | | precursor | |
| 1201 | AY056047 | 5200 | 99 | receptor protein tyrosine kinase | Homo sapiens |
| | | | | EphB4 | |
| 1201 | U07695 | 5196 | 99 | tyrosine kinase | Homo sapiens |
| 1202 | AF015553 | 4955 | 99 | TFII-I protein | Homo sapiens |
| 1202 | AF038969 | 4949 | 99 | general transcription factor 2-I | Homo sapiens |
| 1202 | Y14946 | 4949 | 99 | SPIN protein | Homo sapiens |
| 1203 | AAH07452 | 1442 | 100 | BC007452 Similar to WW domain | Homo sapiens |
| | | | | binding protein 2 | |
| 1203 | AAH10616 | 1442 | 100 | BC010616 Unknown (protein for | Homo sapiens |
| | | | | MGC: 18269) | |
| 1203 | U79458 | 1458 | 100 | WW domain binding protein-2 | Homo sapiens |
| 1204 | AB097511 | 3996 | 93 | hypothetical protein | Macaca |
| | | | | | fascicularis |
| 1204 | AL359292 | 7373 | 99 | dJ448K1.1.1 (absent in melanoma | Homo sapiens |
| | | | | 1, isoform 1) | |
| 1204 | U83115 | 8577 | 100 | non-lens beta gamma-crystallin | Homo sapiens |
| | | | | like protein | |
| 1205 | AAH05921 | 589 | 87 | BC005921 chorionic | Homo sapiens |
| | | | | somatomammotropin hormone 1 | |
| | | | | (placental lactogen) | |
| 1205 | AAH20756 | 589 | 87 | BC020756 chorionic | Homo sapiens |
| | | | | somatomammotropin hormone 1 | |
| | | | | (placental lactogen) | |
| 1205 | AAH22044 | 589 | 87 | BC022044 chorionic | Homo sapiens |
| | | | | somatomammotropin hormone 2 | |
| 1206 | AY158924 | 650 | 100 | histone protein Hist2h2aa2 | Mus musculus |
| 1206 | AY158925 | 650 | 100 | histone protein Hist2h2aa1 | Mus musculus |
| 1206 | AY158953 | 650 | 100 | histone protein Hist2h3c2 | Mus musculus |
| 1207 | AF226614_1 | 2929 | 100 | AF226614 ferroportin1 | Homo sapiens |
| 1207 | AF231121_1 | 2929 | 100 | AF231121 iron-regulated | Homo sapiens |
| | | | | transporter IREG1 | |
| 1207 | BC037733 | 2929 | 100 | solute carrier family 11 (proton- | Homo sapiens |
| | | | | coupled divalent metal ion | |
| | | | | transporters), member 3 | |
| 1208 | AAH01693 | 709 | 100 | BC001693 lectin, galactoside- | Homo sapiens |
| | | | | binding, soluble, 1 (galectin 1) | |
| 1208 | AAH20675 | 709 | 100 | BC020675 lectin, galactoside- | Homo sapiens |
| | | | | binding, soluble, 1 (galectin 1) | |
| 1208 | X14829 | 709 | 100 | beta-galactoside-binding lectin | Homo sapiens |
| | | | | (AA 1-135) | |
| 1209 | AF151794 | 1678 | 38 | pol protein | Phascolarctos |
| | | | | | cinereus |
| 1209 | AY099324 | 1671 | 38 | gag-pol polyprotein | porcine |
| | | | | | endogenous |
| | | | | | retrovirus B |
| 1209 | M26927 | 1717 | 38 | pol polyprotein | Gibbon ape |
| | | | | | leukemia |
| | | | | | virus |
| 1210 | AAD18076 | 689 | 99 | AF129756 G6c | Homo sapiens |
| 1210 | AJ012008 | 689 | 99 | Ly6-C protein | Homo sapiens |
| 1210 | AJ315533 | 689 | 99 | LY6G6C protein | Homo sapiens |
| 1211 | AAH11682 | 2559 | 100 | BC011682 Similar to cathepsin F | Homo sapiens |
| 1211 | AF132894_1 | 2559 | 100 | AF132894 cathepsin F | Homo sapiens |
| 1211 | AJ007331 | 2559 | 100 | cysteine proteinase | Homo sapiens |
| 1212 | AF247565_1 | 513 | 97 | AF247565 anaphase promoting | Homo sapiens |
| | | | | complex subunit 11 | |
| 1212 | AF247789_1 | 513 | 97 | AF247789 putative anaphase- | Homo sapiens |
| | | | | promoting complex subunit APC11 | |
| 1212 | AX061622 | 513 | 97 | unnamed protein product | Homo sapiens |
| 1213 | AAH11811 | 559 | 92 | BC011811 Unknown (protein for | Homo sapiens |
| | | | | MGC: 20260) | |
| 1213 | AF218016_1 | 432 | 76 | AF218016 unknown | Homo sapiens |
| 1214 | AAH17201 | 680 | 92 | BC017201 insulin-like growth | Homo sapiens |
| | | | | factor binding protein 7 | |
| 1214 | L19182 | 680 | 92 | MAC25 | Homo sapiens |
| 1214 | S75725 | 680 | 92 | prostacyclin-stimulating factor; | Homo sapiens |
| | | | | PGI2-stimulating factor; PSF | |
| 1215 | AAH00590 | 250 | 71 | BC000590 actin related protein | Homo sapiens |
| | | | | 2/3 complex, subunit 2 (34 kD) | |
| 1215 | AP006085 | 250 | 71 | p34-Arc | Homo sapiens |
| 1215 | U50523 | 250 | 71 | unknown | Homo sapiens |
| 1216 | AF126110_1 | 718 | 96 | AF126110 fibulin-1 isoform D | Homo sapiens |
| | | | | precursor | |
| 1216 | AF217999_1 | 718 | 96 | AF217999 unknown | Homo sapiens |
| 1216 | BC022497 | 718 | 96 | fibulin 1 | Homo sapiens |
| 1217 | AAH05839 | 502 | 97 | BC005839 follistatin-like 3 | Homo sapiens |
| | | | | (secreted glycoprotein) | |
| 1217 | BC033119 | 502 | 97 | follistatin-like 3 (secreted | Homo sapiens |
| | | | | glycoprotein) | |
| 1217 | U76702 | 502 | 97 | follistatin-related protein FLRG | Homo sapiens |
| 1218 | AAH00163 | 601 | 99 | BC000163 vimentin | Homo sapiens |
| 1218 | BC030573 | 601 | 99 | Unknown (protein for MGC: 16183) | Homo sapiens |
| 1218 | X56134 | 601 | 99 | vimentin | Homo sapiens |
| 1219 | AB018265 | 5555 | 100 | KIAA0722 protein | Homo sapiens |
| 1219 | AF045458 | 5460 | 100 | serine/threonine kinase ULK1 | Homo sapiens |
| 1219 | AF072370_1 | 4865 | 89 | AF072370 UNC51.1 serine/threonine | Mus musculus |
| | | | | kinase | |
| 1220 | AAH00865 | 227 | 100 | BC000865 Unknown (protein for | Homo sapiens |
| | | | | IMAGE: 3460093) | |
| 1221 | AAH02978 | 279 | 91 | BC002978 CD81 antigen (target of | Homo sapiens |
| | | | | antiproliferative antibody 1) | |
| 1221 | AF116600 | 279 | 91 | CD81 | Pan |
| | | | | | troglodytes |
| 1221 | M33680 | 279 | 91 | 26-kDa cell surface protein TAPA-1 | Homo sapiens |
| 1222 | AAH15156 | 678 | 100 | BC015156 ferritin, heavy | Homo sapiens |
| | | | | polypeptide 1 | |
| 1222 | AAH16009 | 678 | 100 | BC016009 ferritin, heavy | Homo sapiens |
| | | | | polypeptide 1 | |
| 1222 | AAH16857 | 678 | 100 | BC016857 ferritin, heavy | Homo sapiens |
| | | | | polypeptide 1 | |
| 1223 | AAH08012 | 518 | 100 | BC008012 eukaryotic translation | Homo sapiens |
| | | | | elongation factor 1 delta | |
| | | | | (guanine nucleotide exchange | |
| | | | | protein) | |
| 1223 | AAH09907 | 518 | 100 | BC009907 eukaryotic translation | Homo sapiens |
| | | | | elongation factor 1 delta | |
| | | | | (guanine nucleotide exchange | |
| | | | | protein) | |
| 1223 | AAH12819 | 518 | 100 | BC012819 eukaryotic translation | Homo sapiens |
| | | | | elongation factor 1 delta | |
| | | | | (guanine nucleotide exchange | |
| | | | | protein) | |
| 1224 | AAH05921 | 556 | 83 | BC005921 chorionic | Homo sapiens |
| | | | | somatomammotropin hormone 1 | |
| | | | | (placental lactogen) | |
| 1224 | AAH20756 | 556 | 83 | BC020756 chorionic | Homo sapiens |
| | | | | somatomammotropin hormone 1 | |
| | | | | (placental lactogen) | |
| 1224 | AAH22044 | 556 | 83 | BC022044 chorionic | Homo sapiens |
| | | | | somatomammotropin hormone 2 | |
| 1225 | A65264 | 705 | 100 | unnamed protein product | unidentified |
| 1225 | AAH01288 | 574 | 93 | BC001288 Similar to decay | Homo sapiens |
| | | | | accelerating factor for | |
| | | | | complement (CD55, Cromer blood | |
| | | | | group system) | |
| 1225 | AF149760_1 | 574 | 93 | AF149760 decay-accelerating | Pan |
| | | | | factor | troglodytes |
| 1226 | AK022217 | 191 | 76 | unnamed protein product | Homo sapiens |
| 1226 | AK056734 | 191 | 72 | unnamed protein product | Homo sapiens |
| 1226 | AK097342 | 209 | 78 | unnamed protein product | Homo sapiens |
| 1227 | AAH05326 | 499 | 98 | BC005326 ribosomal protein L27a | Homo sapiens |
| 1227 | AAH20169 | 499 | 98 | BC020169 Unknown (protein for | Homo sapiens |
| | | | | IMAGE: 3543815) | |
| 1227 | U14968 | 499 | 98 | ribosomal protein L27a | Homo sapiens |
| 1229 | AAH21297 | 569 | 94 | BC021297 Similar to Dynein heavy | Homo sapiens |
| | | | | chain 64C | |
| 1229 | AB002323 | 569 | 94 | KIAA0325 | Homo sapiens |
| 1229 | AY004877 | 539 | 89 | cytoplasmic dynein heavy chain | Mus musculus |
| 1230 | BC026018 | 643 | 100 | Similar to laminin, beta 1 | Homo sapiens |
| 1230 | M20206 | 643 | 100 | laminin B1 | Homo sapiens |
| 1230 | M55370 | 643 | 100 | laminin B1 | Homo sapiens |
| 1231 | AF061658 | 591 | 91 | cytidine deaminase | Homo sapiens |
| 1231 | AJ000474 | 591 | 91 | cytidine deaminase | Homo sapiens |
| 1231 | L27943 | 591 | 91 | cytidine deaminase | Homo sapiens |
| 1232 | AAH18986 | 486 | 83 | BC018986 Unknown (protein for | Homo sapiens |
| | | | | MGC: 20092) | |
| 1232 | AF229830 | 486 | 83 | prostaglandin dehydrogenase | Papio |
| | | | | | hamadryas |
| 1232 | U63296 | 486 | 83 | 15-hydroxyprostaglandin | Homo sapiens |
| | | | | dehydrogenase | |
| 1233 | AAH00749 | 507 | 100 | BC000749 lactate dehydrogenase A | Homo sapiens |
| 1233 | AAH01829 | 507 | 100 | BC001829 lactate dehydrogenase A | Homo sapiens |
| 1233 | X03077 | 507 | 100 | lactate dehydrogenase-A | Homo sapiens |
| 1234 | AAH00903 | 791 | 88 | BC000903 high-mobility group | Homo sapiens |
| | | | | (nonhistone chromosomal) protein 2 | |
| 1234 | AAH01063 | 791 | 88 | BC001063 high-mobility group | Homo sapiens |
| | | | | (nonhistone chromosomal) protein 2 | |
| 1234 | X62534 | 791 | 88 | HMG-2 | Homo sapiens |
| 1235 | AF193055_1 | 594 | 88 | AF193055 unknown | Homo sapiens |
| 1235 | AK075214 | 594 | 88 | unnamed protein product | Homo sapiens |
| 1235 | BC038587 | 594 | 88 | Similar to EGF-like-domain, | Homo sapiens |
| | | | | multiple 6 | |
| 1236 | AF135060 | 632 | 96 | fibrillin-2 | Rattus |
| | | | | | norvegicus |
| 1236 | U03272 | 637 | 99 | fibrillin-2 | Homo sapiens |
| 1236 | X62009 | 637 | 98 | fibrillin 5 | Homo sapiens |
| 1237 | AAH13083 | 522 | 100 | BC013083 Similar to cystatin C | Homo sapiens |
| | | | | (amyloid angiopathy and cerebral | |
| | | | | hemorrhage) | |
| 1237 | X12763 | 522 | 100 | ompA —cystatin C fusion | synthetic |
| | | | | preprotein (AA −21 to 120) | construct |
| 1237 | X61681 | 522 | 100 | cystatin C | Homo sapiens |
| 1239 | AF135060 | 523 | 89 | fibrillin-2 | Rattus |
| | | | | | norvegicus |
| 1239 | L39790 | 532 | 90 | fibrillin 2 | Mus musculus |
| 1239 | U03272 | 597 | 100 | fibrillin-2 | Homo sapiens |
| 1240 | M65149 | 1200 | 85 | CELF | Rattus |
| | | | | | norvegicus |
| 1240 | M83667 | 1430 | 100 | NF-IL6-beta protein | Homo sapiens |
| 1240 | S63168 | 1418 | 99 | CCAAT/enhancer-binding protein | Homo sapiens |
| | | | | delta; C/EBP delta | |
| 1241 | AF186111_1 | 230 | 52 | AF186111 NOTCH4-like protein | Homo sapiens |
| 1241 | AL512735 | 230 | 52 | hypothetical protein | Homo sapiens |
| 1241 | AX133831 | 230 | 52 | unnamed protein product | Homo sapiens |
| 1242 | BC015302 | 664 | 87 | lamin A | Mus musculus |
| 1242 | M13452 | 715 | 95 | lamin A protein | Homo sapiens |
| 1242 | X76297 | 670 | 88 | lamin A | Rattus |
| | | | | | norvegicus |
| 1244 | AB049946 | 666 | 100 | mitochondrial ribosomal protein | Homo sapiens |
| | | | | S15 | |
| 1244 | BC029193 | 433 | 69 | mitochondrial ribosomal protein | Mus musculus |
| | | | | S15 | |
| 1244 | BC031336 | 666 | 100 | mitochondrial ribosomal protein | Homo sapiens |
| | | | | S15 | |
| 1245 | AAH13733 | 392 | 93 | BC013733 px19-like protein | Homo sapiens |
| 1245 | AAH13748 | 392 | 93 | BC013748 px19-like protein | Homo sapiens |
| 1245 | AF201925_1 | 392 | 93 | AF201925 PRELI | Homo sapiens |
| 1246 | AAH02362 | 694 | 100 | BC002362 lactate dehydrogenase B | Homo sapiens |
| 1246 | AAH15122 | 694 | 100 | BC015122 lactate dehydrogenase B | Homo sapiens |
| 1246 | Y00711 | 694 | 100 | lactate dehydrogenase B (AA 1-334) | Homo sapiens |
| 1247 | AAH03070 | 504 | 86 | BC003070 GATA-binding protein 3 | Homo sapiens |
| 1247 | AAH06793 | 504 | 86 | BC006793 GATA-binding protein 3 | Homo sapiens |
| 1247 | X55037 | 504 | 86 | GATA-3 | Homo sapiens |
| 1248 | AF269289_1 | 357 | 100 | AF269289 unknown | Homo sapiens |
| 1249 | AAH07728 | 546 | 86 | BC007728 Unknown (protein for | Homo sapiens |
| | | | | MGC: 12671) | |
| 1249 | AL080102 | 546 | 86 | hypothetical protein | Homo sapiens |
| 1249 | U49436 | 546 | 86 | translation initiation factor 5 | Homo sapiens |
| 1250 | AAH03190 | 472 | 94 | BC003190 p75NTR-associated cell | Homo sapiens |
| | | | | death executor; ovarian granulosa | |
| | | | | cell protein (13 kD) | |
| 1250 | AF187064_1 | 472 | 94 | AF187064 p75NTR-associated cell | Homo sapiens |
| | | | | death executor; NADE | |
| 1250 | M38188 | 472 | 94 | unknown | Homo sapiens |
| 1251 | AB037767 | 5427 | 99 | KIAA1346 protein | Homo sapiens |
| 1251 | AF207664_1 | 5274 | 100 | AF207664 matrix metalloprotease | Homo sapiens |
| 1251 | AP001697 | 5274 | 100 | metalloprotease with | Homo sapiens |
| | | | | thrombospondin type 1 motifs | |
| 1252 | U90938 | 222 | 76 | Fc gamma receptor IIc1 | Homo sapiens |
| 1252 | X17652 | 222 | 76 | IgG Fc receptor | Homo sapiens |
| 1252 | X17652 | 222 | 76 | IgG Fc receptor | Homo sapiens |
| 1253 | AAH01768 | 390 | 93 | BC001768 neuronatin | Homo sapiens |
| 1253 | AL109614 | 390 | 93 | bA425M5.3.1 (neuronatin (isoform | Homo sapiens |
| | | | | 1)) | |
| 1253 | U31767 | 390 | 93 | neuronatin alpha | Homo sapiens |
| 1254 | AF151373_1 | 542 | 75 | AF151373 nucleolin-related | Rattus |
| | | | | protein NRP | norvegicus |
| 1254 | AK091742 | 670 | 96 | unnamed protein product | Homo sapiens |
| 1254 | M60858 | 670 | 96 | nucleolin | Homo sapiens |
| 1255 | AAH13428 | 616 | 93 | BC013428 PP1201 protein | Homo sapiens |
| 1255 | AF193045_1 | 616 | 93 | AF193045 unknown | Homo sapiens |
| 1255 | BC026348 | 616 | 93 | PP1201 protein | Homo sapiens |
| 1256 | AF393832_1 | 765 | 99 | AF393832 beta-actin | Morulius |
| | | | | | calbasu |
| 1256 | AY039657 | 765 | 99 | beta-actin | Chrysophrys |
| | | | | | auratus |
| 1256 | AY148350 | 765 | 99 | actin | Dicentrarchus |
| | | | | | labrax |
| 1257 | AK055593 | 379 | 100 | unnamed protein product | Homo sapiens |
| 1258 | AAH21233 | 1036 | 100 | BC021233 ATP synthase, H+ | Homo sapiens |
| | | | | transporting, mitochondrial F1 | |
| | | | | complex, O subunit (oligomycin | |
| | | | | sensitivity conferring protein) | |
| 1258 | BC022865 | 1036 | 100 | ATP synthase, H+ transporting, | Homo sapiens |
| | | | | mitochondrial F1 complex, O | |
| | | | | subunit (oligomycin sensitivity | |
| | | | | conferring protein) | |
| 1258 | X83218 | 1036 | 100 | ATP synthase, oligomycin | Homo sapiens |
| | | | | sensitivity conferring protein | |
| 1259 | AAH04368 | 1221 | 99 | BC004368 proteasome (prosome, | Homo sapiens |
| | | | | macropain) activator subunit 2 | |
| | | | | (PA28 beta) | |
| 1259 | AAH19885 | 1221 | 99 | BC019885 proteasome (prosome, | Homo sapiens |
| | | | | macropain) activator subunit 2 | |
| | | | | (PA28 beta) | |
| 1259 | D45248 | 1226 | 100 | proteasome activator hPA28 suunit | Homo sapiens |
| | | | | beta | |
| 1260 | AF090306 | 2306 | 100 | retinoblastoma binding protein | Rattus |
| | | | | | norvegicus |
| 1260 | U35143 | 2306 | 100 | retinoblastoma-binding protein | Homo sapiens |
| | | | | RbAp46 | |
| 1260 | X72841 | 2306 | 100 | IEF 7442 | Homo sapiens |
| 1261 | AAH00413 | 1748 | 100 | BC000413 eukaryotic translation | Homo sapiens |
| | | | | initiation factor 3, subunit 2 | |
| | | | | (beta, 36 kD) | |
| 1261 | AAH03140 | 1748 | 100 | BC003140 eukaryotic translation | Homo sapiens |
| | | | | initiation factor 3, subunit 2 | |
| | | | | (beta, 36 kD) | |
| 1261 | U39067 | 1748 | 100 | translation initiation factor | Homo sapiens |
| | | | | eIF3 p36 subunit | |
| 1262 | AF042166 | 13721 | 100 | beta-filamin | Homo sapiens |
| 1262 | AF043045 | 13709 | 99 | actin-binding protein homolog | Homo sapiens |
| | | | | ABP-278 | |
| 1262 | AF191633 | 13721 | 100 | filamin | Homo sapiens |
| 1263 | AF165515_1 | 1862 | 100 | AF165515 ancient ubiquitous | Homo sapiens |
| | | | | protein 1 precursor | |
| 1263 | AK023983 | 1935 | 100 | unnamed protein product | Homo sapiens |
| 1263 | BC033646 | 1862 | 100 | ancient ubiquitous protein 1 | Homo sapiens |
| 1264 | AB009865 | 1347 | 100 | Angiopoietin-2 | Homo sapiens |
| 1264 | AF187858_1 | 1335 | 99 | AF187858 angiopoietin-2 isoform-1 | Homo sapiens |
| 1264 | AF218015_1 | 1347 | 100 | AF218015 unknown | Homo sapiens |
| 1265 | AF035718 | 914 | 99 | mesoderm-specific basic-helix- | Homo sapiens |
| | | | | loop-helix protein; Pod-1 | |
| 1265 | AL356109 | 917 | 100 | bA373A10.1 (transcription factor | Homo sapiens |
| | | | | 21) | |
| 1265 | BC025697 | 917 | 100 | transcription factor 21 | Homo sapiens |
| 1266 | M38690 | 1200 | 100 | CD9 antigen | Homo sapiens |
| 1266 | S60489 | 1200 | 100 | CD9 antigen | Homo sapiens |
| 1266 | X60111 | 1200 | 100 | MRP-1 (motility related protein) | Homo sapiens |
| 1267 | AAH02368 | 4629 | 99 | BC002368 proteasome (prosome, | Homo sapiens |
| | | | | macropain) 26S subunit, non- | |
| | | | | ATPase, 2 | |
| 1267 | AAH02997 | 4629 | 99 | BC002997 proteasome (prosome, | Homo sapiens |
| | | | | macropain) 26S subunit, non- | |
| | | | | ATPase, 2 | |
| 1267 | D78151 | 4637 | 100 | human 26S proteasome subunit p97 | Homo sapiens |
| 1268 | AAH00182 | 1622 | 100 | BC000182 annexin A4 | Homo sapiens |
| 1268 | AAH11659 | 1622 | 100 | BC011659 Similar to annexin A4 | Homo sapiens |
| 1268 | D78152 | 1622 | 100 | annexin IV (carbohydrtate-binding | Homo sapiens |
| | | | | protein p33/41) | |
| 1269 | AAH03064 | 4016 | 99 | BC003064 disabled (Drosophila) | Homo sapiens |
| | | | | homolog 2 (mitogen-responsive | |
| | | | | phosphoprotein) | |
| 1269 | AF205890 | 4028 | 100 | disabled-2 | Homo sapiens |
| 1269 | U39050 | 4028 | 100 | DOC-2 | Homo sapiens |
| 1270 | AAH07075 | 780 | 100 | BC007075 hemoglobin, beta | Homo sapiens |
| 1270 | U01317 | 780 | 100 | beta-globin | Homo sapiens |
| 1270 | V00499 | 780 | 100 | beta globin | Homo sapiens |
| 1271 | AAH21557 | 4093 | 99 | BC021557 transmembrane protein 8 | Homo sapiens |
| | | | | (five membrane-spanning domains) | |
| 1271 | AB045292 | 4100 | 100 | M83 protein | Homo sapiens |
| 1271 | AE006463_7 | 4088 | 99 | AE006463 M83 | Homo sapiens |
| 1272 | AF193048_1 | 946 | 100 | AF193048 unknown | Homo sapiens |
| 1273 | AF151980_1 | 2009 | 100 | AF151980 connexin 43 | Homo sapiens |
| 1273 | BC026329 | 2009 | 100 | gap junction protein, alpha 1, | Homo sapiens |
| | | | | 43 kD (connexin 43) | |
| 1273 | X52947 | 2009 | 100 | gap junction protein (AA 1-382) | Homo sapiens |
| 1274 | AJ313463 | 1258 | 100 | adipsin/complement factor D | Homo sapiens |
| | | | | precursor | |
| 1274 | BC034529 | 1249 | 100 | Unknown (protein for | Homo sapiens |
| | | | | IMAGE: 4780594) | |
| 1274 | M84526 | 1198 | 98 | adipsin/complement factor D | Homo sapiens |
| 1275 | AF258549_1 | 533 | 100 | AF258549 PP1292 | Homo sapiens |
| 1276 | AF217963_1 | 4215 | 99 | AF217963 NRAGE | Homo sapiens |
| 1276 | AF258554_1 | 4223 | 100 | AF258554 PP2250 | Homo sapiens |
| 1276 | BC032473 | 4223 | 100 | melanoma antigen, family D, 1 | Homo sapiens |
| 1277 | AAH14635 | 635 | 100 | BC014635 Similar to SH3-domain, | Homo sapiens |
| | | | | GRB2-like, endophilin B2 | |
| 1277 | AF257319_1 | 635 | 100 | AF257319 SH3-containing protein | Homo sapiens |
| | | | | SH3GLB2 | |
| 1277 | AF258589_1 | 635 | 100 | AF258589 PP578 | Homo sapiens |
| 1278 | AAH03390 | 707 | 100 | BC003390 hypothetical protein | Homo sapiens |
| 1278 | AAH14334 | 707 | 100 | BC014334 Unknown (protein for | Homo sapiens |
| | | | | MGC: 22874) | |
| 1278 | AF275807_1 | 822 | 100 | AF275807 PNAS-110 | Homo sapiens |
| 1279 | AAH05238 | 469 | 100 | BC005238 FXYD domain-containing | Homo sapiens |
| | | | | ion transport regulator 3 | |
| 1279 | U28249 | 411 | 75 | 11 kD protein | Homo sapiens |
| 1279 | X93036 | 469 | 100 | MAT8 protein | Homo sapiens |
| 1280 | AF014402 | 1500 | 100 | type-2 phosphatidic acid | Homo sapiens |
| | | | | phosphatase alpha-1 | |
| 1280 | BC039847 | 1500 | 100 | Similar to phosphatidic acid | Homo sapiens |
| | | | | phosphatase type 2A | |
| 1280 | Y14436 | 1500 | 100 | phosphatidic acid phosphatase | Homo sapiens |
| | | | | type 2 | |
| 1281 | AL163249 | 2729 | 99 | T-complex protein 1 theta subunit | Homo sapiens |
| 1281 | D13627 | 2730 | 99 | KIAA0002 | Homo sapiens |
| 1281 | D42052 | 2734 | 100 | predicted protein of 548 amino | Homo sapiens |
| | | | | acids | |
| 1282 | AAH01312 | 2306 | 100 | BC001312 protein disulfide | Homo sapiens |
| | | | | isomerase-related protein | |
| 1282 | BC006865 | 2220 | 95 | Similar to protein disulfide | Mus musculus |
| | | | | isomerase-related protein | |
| 1283 | D49489 | 2306 | 100 | human P5 | Homo sapiens |
| 1283 | AF439513_1 | 7917 | 91 | AF439513 pregnancy-associated | Mus musculus |
| | | | | plasma protein-A | |
| 1283 | U28727 | 8980 | 100 | pregnancy-associated plasma | Homo sapiens |
| | | | | protein-A preproform | |
| 1283 | X68280 | 8574 | 100 | unnamed protein product | Homo sapiens |
| 1284 | AAH01936 | 1357 | 100 | BC001936 Similar to BCL2- | Homo sapiens |
| | | | | associated athanogene | |
| 1284 | AAH14774 | 1357 | 100 | BC014774 Unknown (protein for | Homo sapiens |
| | | | | MGC: 17086) | |
| 1284 | Z35491 | 1357 | 100 | glucocortoid receptor-associated | Homo sapiens |
| | | | | protein RAP46 | |
| 1285 | AAH04490 | 1953 | 100 | BC004490 v-fos FBJ murine | Homo sapiens |
| | | | | osteosarcoma viral oncogene | |
| | | | | homolog | |
| 1285 | AF111167 | 1953 | 100 | cfos | Homo sapiens |
| 1285 | V01512 | 1953 | 100 | c-fos | Homo sapiens |
| 1286 | AAH20235 | 3282 | 99 | BC020235 Unknown (protein for | Homo sapiens |
| | | | | MGC: 31939) | |
| 1286 | M19645 | 3314 | 100 | GRP78 precursor | Homo sapiens |
| 1286 | X87949 | 3314 | 100 | BiP | Homo sapiens |
| 1287 | AAH14433 | 2640 | 100 | BC014433 Unknown (protein for | Homo sapiens |
| | | | | MGC: 2159) | |
| 1287 | BC036000 | 2640 | 100 | Unknown (protein for | Homo sapiens |
| | | | | IMAGE: 4712175) | |
| 1287 | U42068 | 2640 | 100 | P58 | Homo sapiens |
| 1288 | BC032722 | 1478 | 100 | tumor necrosis factor (ligand) | Homo sapiens |
| | | | | superfamily, member 10 | |
| 1288 | U37518 | 1478 | 100 | TNF-related apoptosis inducing | Homo sapiens |
| | | | | ligand TRAIL | |
| 1288 | U57059 | 1478 | 100 | Apo-2 ligand | Homo sapiens |
| 1289 | AAH01022 | 1591 | 100 | BC001022 pyrophosphatase | Homo sapiens |
| | | | | (inorganic) | |
| 1289 | AF119665_1 | 1591 | 100 | AF119665 inorganic | Homo sapiens |
| | | | | pyrophosphatase | |
| 1289 | AF217186_1 | 1591 | 100 | AF217186 inorganic | Homo sapiens |
| | | | | pyrophosphatase 1 | |
| 1290 | AAH08743 | 3145 | 100 | BC008743 zyxin | Homo sapiens |
| 1290 | AAH09360 | 3145 | 100 | BC009360 zyxin | Homo sapiens |
| 1290 | AAH10031 | 3145 | 100 | BC010031 zyxin | Homo sapiens |
| 1291 | AAH05901 | 919 | 100 | BC005901 Microfibril-associated | Homo sapiens |
| | | | | glycoprotein-2 | |
| 1291 | AF084927 | 919 | 100 | microfibril-associated | Homo sapiens |
| | | | | glycoprotein 2 | |
| 1291 | U37283 | 919 | 100 | microfibril-associated | Homo sapiens |
| | | | | glycoprotein-2 MAGP-2 | |
| 1292 | AAH00933 | 2015 | 100 | BC000933 isocitrate dehydrogenase | Homo sapiens |
| | | | | 3 (NAD+) gamma | |
| 1292 | Z68129 | 2015 | 100 | NAD (H)-specific isocitrate | Homo sapiens |
| | | | | dehydrogenase gamma-subunit | |
| | | | | precursor | |
| 1292 | Z68907 | 2015 | 100 | NAD (H)-specific isocitrate | Homo sapiens |
| | | | | dehydrogenase gamma subunit | |
| | | | | precursor | |
| 1293 | AAH12265 | 846 | 100 | BC012265 Similar to cofilin 1, | Homo sapiens |
| | | | | non-muscle | |
| 1293 | AAH12318 | 846 | 100 | BC012318 Similar to cofilin 1, | Homo sapiens |
| | | | | non-muscle | |
| 1293 | AAH18256 | 846 | 100 | BC018256 Similar to cofilin 1, | Homo sapiens |
| | | | | non-muscle | |
| 1294 | AB069964 | 861 | 100 | ubiquitin-conjugating enzyme 9 | Gallus |
| | | | | | gallus |
| 1294 | AF461016_1 | 861 | 100 | AF461016 ubiquitin-conjugating | Gallus |
| | | | | enzyme | gallus |
| 1294 | U88561 | 861 | 100 | E2 ubiquitin conjugating enzyme | Xenopus |
| | | | | | laevis |
| 1295 | AAH06249 | 989 | 100 | BC006249 guanylate kinase 1 | Homo sapiens |
| 1295 | AAH09914 | 989 | 100 | BC009914 guanylate kinase 1 | Homo sapiens |
| 1295 | U66895 | 989 | 100 | guanylate kinase | Homo sapiens |
| 1296 | AF095770_1 | 224 | 94 | AF095770 PTH-responsive | Homo sapiens |
| | | | | osteosarcoma D1 protein | |
| 1297 | AAH01120 | 1369 | 99 | BC001120 lectin, galactoside- | Homo sapiens |
| | | | | binding, soluble, 3 (galectin 3) | |
| 1297 | AF031425 | 1369 | 99 | galectin 3 | Homo sapiens |
| 1297 | M35368 | 1377 | 100 | galactose-specific lectin | Homo sapiens |
| 1298 | AF241786_1 | 1125 | 100 | AF241786 NPD013 | Homo sapiens |
| 1299 | AAH02503 | 547 | 70 | BC002503 spermidine/spermine N1- | Homo sapiens |
| | | | | acetyltransferase | |
| 1299 | AAH08424 | 547 | 70 | BC008424 spermdine/spermine N1- | Homo sapiens |
| | | | | acetyltransferase | |
| 1299 | AF251292_1 | 1059 | 100 | AF251292 DC21 | Homo sapiens |
| 1300 | AF135157_1 | 190 | 63 | AF135157 complement C1q A chain | Homo sapiens |
| | | | | precursor | |
| 1300 | AF260332_1 | 1550 | 100 | AF260332 DC33 | Homo sapiens |
| 1300 | BC030153 | 190 | 63 | complement component 1, q | Homo sapiens |
| | | | | subcomponent, alpha polypeptide | |
| 1301 | AAH00589 | 770 | 100 | BC000589 CGI-39 protein; cell | Homo sapiens |
| | | | | death-regulatory protein GRIM19 | |
| 1301 | AAH09189 | 770 | 100 | BC009189 CGI-39 protein; cell | Homo sapiens |
| | | | | death-regulatory protein GRIM19 | |
| 1301 | AF261134_1 | 1196 | 100 | AF261134 CDA016 | Homo sapiens |
| 1302 | AAH12296 | 1135 | 99 | BC012296 hypothetical protein | Homo sapiens |
| | | | | FLJ21174 | |
| 1302 | AF271783_1 | 1141 | 100 | AF271783 NPD017 | Homo sapiens |
| 1302 | AF314542_1 | 1141 | 100 | AF314542 B lymphocyte activation- | Homo sapiens |
| | | | | related protein | |
| 1303 | AK090427 | 13940 | 99 | FLJ00343 protein | Homo sapiens |
| 1303 | L44140 | 14011 | 100 | filamin | Homo sapiens |
| 1303 | X53416 | 14004 | 99 | actin-binding protein | Homo sapiens |
| 1304 | AAH12341 | 1492 | 100 | BC012341 Similar to M5-14 protein | Homo sapiens |
| 1304 | AL136622 | 1492 | 100 | hypothetical protein | Homo sapiens |
| 1304 | AL390090 | 1492 | 100 | c3orf1 hypothetical protein, M5-14 | Homo sapiens |
| | | | | similar to (AE003703) 140up | |
| | | | | gene product Drosophila | |
| | | | | melanogaster | |
| 1305 | AC005624 | 824 | 100 | MY18_HUMAN | Homo sapiens |
| 1305 | AF078077 | 824 | 100 | growth arrest and DNA-damage- | Homo sapiens |
| | | | | inducible protein GADD45beta | |
| 1305 | AF087853_1 | 824 | 100 | AF087853 growth arrest and DNA | Homo sapiens |
| | | | | damage inducible protein beta | |
| 1306 | AF020185 | 478 | 100 | protein inhibitor of nitric oxide | Mus musculus |
| | | | | synthase | |
| 1306 | BC008106 | 478 | 100 | dynein, cytoplasmic, light | Mus musculus |
| | | | | polypeptide | |
| 1306 | BC034258 | 478 | 100 | dynein, cytoplasmic, light | Mus musculus |
| | | | | polypeptide | |
| 1307 | AAH01539 | 1466 | 100 | BC001539 dickkopf (Xenopus | Homo sapiens |
| | | | | laevis) homolog 1 | |
| 1307 | AF177394_1 | 1466 | 100 | AF177394 dickkopf-1 | Homo sapiens |
| 1307 | AF261158 | 1466 | 100 | dickkopf homolog 1 | Homo sapiens |
| 1308 | AAC33279 | 1396 | 100 | AC005559 basigin | Homo sapiens |
| 1308 | AAH09040 | 1396 | 100 | BC009040 basigin (OK blood group) | Homo sapiens |
| 1308 | AF042855 | 1396 | 100 | EMMPRIN | Homo sapiens |
| 1309 | L47125 | 3057 | 100 | glypican | Homo sapiens |
| 1309 | L47176 | 3057 | 100 | GTR2-2 gene product | Homo sapiens |
| 1309 | Z37987 | 3057 | 100 | MXR7 | Homo sapiens |
| 1310 | D83476 | 4306 | 77 | xtld protein | Xenopus |
| | | | | | laevis |
| 1310 | L24755 | 4948 | 92 | bone morphogenetic protein | Mus musculus |
| 1310 | U50330 | 5416 | 100 | procollagen C-proteinase | Homo sapiens |
| 1311 | AAH01287 | 669 | 100 | BC001287 histidine triad | Homo sapiens |
| | | | | nucleotide-binding protein | |
| 1311 | AAH07090 | 669 | 100 | BC007090 histidine triad | Homo sapiens |
| | | | | nucleotide-binding protein | |
| 1311 | U51004 | 669 | 100 | protein kinase C inhibitor | Homo sapiens |
| 1312 | AB003306 | 1280 | 94 | PSMB5 | Mus musculus |
| 1312 | AF060091_1 | 1280 | 94 | AF060091 proteasome subunit X | Mus musculus |
| 1312 | X95586 | 1370 | 100 | proteasome | Homo sapiens |
| 1313 | AF102848_1 | 2178 | 100 | AF102848 keratin 23 | Homo sapiens |
| 1313 | AK002047 | 2151 | 98 | unnamed protein product | Homo sapiens |
| 1313 | BC028356 | 2155 | 99 | type I intermediate filament | Homo sapiens |
| | | | | cytokeratin | |
| 1314 | AAH00097 | 3483 | 100 | BC000097 transforming growth | Homo sapiens |
| | | | | factor, beta-induced, 68 kD | |
| 1314 | AAR04972 | 3483 | 100 | BC004972 transforming growth | Homo sapiens |
| | | | | factor, beta-induced, 68 kD | |
| 1314 | AY149344 | 3483 | 100 | transforming growth factor, beta- | Homo sapiens |
| | | | | induced, 68 kDa | |
| 1315 | AF414110_1 | 635 | 100 | AF414110 histone variant H2A.F/Z | Danio rerio |
| 1315 | AF414111_1 | 635 | 100 | AF414111 histone variant H2A.F/Z | Danio rerio |
| 1315 | V00414 | 635 | 100 | histone H2A | Gallus |
| | | | | | gallus |
| 1316 | BC011457 | 957 | 100 | Unknown (protein for MGC: 7976) | Mus musculus |
| 1316 | BC019761 | 957 | 100 | putative membrane protein | Mus musculus |
| 1316 | BC020098 | 957 | 100 | putative membrane protein | Mus musculus |
| 1317 | AAH00140 | 3579 | 100 | BC000140 propionyl Coenzyme A | Homo sapiens |
| | | | | carboxylase, alpha polypeptide | |
| 1317 | AF385926_1 | 3579 | 100 | AF385926 propionyl-CoA | Homo sapiens |
| | | | | carboxylase alpha subunit | |
| 1317 | AY035808 | 3579 | 100 | propionyl-CoA carboxylase alpha | Homo sapiens |
| | | | | polypeptide precursor | |
| 1318 | AF372216_1 | 562 | 99 | AF372216 tropomyosin alpha | Rattus |
| | | | | isoform | norvegicus |
| 1318 | M19267 | 565 | 100 | tropomyosin | Homo sapiens |
| 1318 | M19715 | 565 | 100 | skeletal muscle tropomyosin | Homo sapiens |
| 1319 | AAH00191 | 704 | 100 | BC000191 hypothetical protein | Homo sapiens |
| 1319 | AAH14329 | 704 | 100 | BC014329 Unknown (protein for | Homo sapiens |
| | | | | MGC: 22862) | |
| 1319 | AF212248_1 | 704 | 100 | AF212248 CDA09 | Homo sapiens |
| 1320 | AAH00255 | 783 | 100 | BC000255 Unknown (protein for | Homo sapiens |
| | | | | MGC: 2495) | |
| 1320 | AF320778_1 | 783 | 100 | AF320778 cervical cancer oncogene 3 | Homo sapiens |
| 1320 | AY032594 | 783 | 100 | hepatitis C virus core-binding | Homo sapiens |
| | | | | protein 6 | |
| 1321 | AAH00271 | 799 | 100 | BC000271 Unknown (protein for | Homo sapiens |
| | | | | MGC: 3204) | |
| 1321 | AAH01434 | 799 | 100 | BC001434 Unknown (protein for | Homo sapiens |
| | | | | MGC: 2477) | |
| 1321 | AK007390 | 449 | 82 | unnamed protein product | Mus musculus |
| 1322 | AAH00421 | 1243 | 100 | BC000421 lysosomal-associated | Homo sapiens |
| | | | | protein transmembrane 4 alpha | |
| 1322 | AAH03158 | 1240 | 99 | BC003158 lysosomal-associated | Homo sapiens |
| | | | | protein transmembrane 4 alpha | |
| 1322 | D14696 | 1243 | 100 | KIAA0108 | Homo sapiens |
| 1323 | AAH00461 | 1738 | 100 | BC000461 eukaryotic translation | Homo sapiens |
| | | | | initiation factor 2, subunit 2 | |
| | | | | (beta, 38 kD) | |
| 1323 | AAH00934 | 1735 | 99 | BC000934 eukaryotic translation | Homo sapiens |
| | | | | initiation factor 2, subunit 2 | |
| | | | | (beta, 38 kD) | |
| 1323 | AL031668 | 1738 | 100 | dJ64K7.2 (eukaryotic translation | Homo sapiens |
| | | | | initiation factor 2, subunit 2 | |
| | | | | (beta, 38 kD)) | |
| 1324 | AAH00466 | 1059 | 100 | BC000466 NADH dehydrogenase | Homo sapiens |
| | | | | (ubiquinone) 1 beta subcomplex, 8 | |
| | | | | (19 kD, ASHI) | |
| 1324 | AAH19276 | 1059 | 100 | BC019276 NADH dehydrogenase | Homo sapiens |
| | | | | (ubiquinone) 1 beta subcomplex, 8 | |
| | | | | (19 kD, ASHI) | |
| 1324 | AF044958 | 1059 | 100 | NADH: ubiquinone oxidoreductase | Homo sapiens |
| | | | | ASHI subunit | |
| 1325 | AAH00490 | 1821 | 100 | BC000490 eukaryotic translation | Homo sapiens |
| | | | | initiation factor 3, subunit 5 | |
| | | | | (epsilon, 47 KD) | |
| 1325 | AK095574 | 1774 | 98 | unnamed protein product | Homo sapiens |
| 1325 | U94855 | 1821 | 100 | translation initiation factor 3 | Homo sapiens |
| | | | | 47 kDa subunit | |
| 1326 | AAH00502 | 970 | 100 | BC000502 ribosomal protein L17 | Homo sapiens |
| 1326 | AAH17831 | 970 | 100 | BC017831 ribosomal protein L17 | Homo sapiens |
| 1326 | X53777 | 970 | 100 | putative ribosomal protein (AA 1-184) | Homo sapiens |
| 1327 | AAH00505 | 808 | 100 | BC000505 microsomal glutathione | Homo sapiens |
| | | | | S-transferase 3 | |
| 1327 | AAH03034 | 808 | 100 | BC003034 microsomal glutathione | Homo sapiens |
| | | | | S-transferase 3 | |
| 1327 | AAH05964 | 808 | 100 | BC005964 microsomal glutathione | Homo sapiens |
| | | | | S-transferase 3 | |
| 1328 | AAH00509 | 1425 | 100 | BC000509 proteasome (prosome, | Homo sapiens |
| | | | | macropain) subunit, beta type, 7 | |
| 1328 | AAH17116 | 1393 | 100 | BC017116 proteasome (prosome, | Homo sapiens |
| | | | | macropain) subunit, beta type, 7 | |
| 1328 | D38048 | 1421 | 99 | proteasome subunit z | Homo sapiens |
| 1329 | BC033015 | 4539 | 100 | RAS p21 protein activator (GTPase | Homo sapiens |
| | | | | activating protein) 1 | |
| 1329 | M23379 | 4539 | 100 | GTPase-activating protein | Homo sapiens |
| 1329 | M23612 | 4555 | 100 | GTPase-activating protein | Homo sapiens |
| 1330 | BC003860 | 2254 | 100 | protease (prosome, macropain) 26S | Mus musculus |
| | | | | subunit, ATPase 1 | |
| 1330 | D50696 | 2254 | 100 | proteasomal ATPase (S4) | Rattus |
| | | | | | norvegicus |
| 1330 | U39302 | 2254 | 100 | P26s4 | Mus musculus |
| 1331 | AAH00522 | 2131 | 100 | BC000522 Similar to serine (or | Homo sapiens |
| | | | | cysteine) proteinase inhibitor, | |
| | | | | clade F (alpha-2 antiplasmin, | |
| | | | | pigment epithelium derived | |
| | | | | factor). member 1 | |
| 1331 | AF400442_1 | 2125 | 99 | AF400442 pigment epithelium- | Homo sapiens |
| | | | | derived factor | |
| 1331 | M76979 | 2119 | 99 | pigment epithelial- | Homo sapiens |
| | | | | differentiating factor | |
| 1332 | AAH00529 | 1616 | 100 | BC000529 prostate differentiation | Homo sapiens |
| | | | | factor | |
| 1332 | AAH08962 | 1616 | 100 | BC008962 Unknown (protein for | Homo sapiens |
| | | | | MGC: 4145) | |
| 1332 | AF003934 | 1613 | 99 | prostate differentiation factor | Homo sapiens |
| 1333 | AAH00548 | 806 | 100 | BC000548 receptor (calcitonin) | Homo sapiens |
| | | | | activity modifying protein 1 | |
| 1333 | AF181550_1 | 596 | 71 | AF181550 receptor activity | Rattus |
| | | | | modifying protein 1 | norvegicus |
| 1333 | AJ001014 | 806 | 100 | RAMP1 | Homo sapiens |
| 1334 | AAH00554 | 1409 | 100 | BC000554 Similar to integral | Homo sapiens |
| | | | | membrane protein 2B | |
| 1334 | AF136973_1 | 1409 | 100 | AF136973 putative transmembrane | Homo sapiens |
| | | | | protein E3-16 | |
| 1334 | AF152462_1 | 1409 | 100 | AF152462 transmembrane protein | Homo sapiens |
| | | | | BRI | |
| 1335 | AAH00601 | 479 | 100 | BC000601 DKFZP564K247 protein | Homo sapiens |
| 1335 | AAH09583 | 479 | 100 | BC009583 DKFZP564K247 protein | Homo sapiens |
| 1335 | AAH09594 | 479 | 100 | BC009594 DKFZP564K247 protein | Homo sapiens |
| 1336 | AAH00691 | 906 | 100 | BC000691 brain specific protein | Homo sapiens |
| 1336 | AF132972_1 | 902 | 99 | AF132972 CGI-38 protein | Homo sapiens |
| 1336 | BC010788 | 888 | 96 | RIKEN cDNA 2700055K07 gene | Mus musculus |
| 1337 | AAH00720 | 798 | 100 | BC000720 ubiquitously-expressed | Homo sapiens |
| | | | | transcript | |
| 1337 | AAH08890 | 798 | 100 | BC008890 ubiquitously-expressed | Homo sapiens |
| | | | | transcript | |
| 1337 | AF092737_1 | 798 | 100 | AF092737 ubiquitously expressed | Homo sapiens |
| | | | | transcript | |
| 1338 | AAH01016 | 958 | 100 | BC001016 NADH dehydrogenase | Homo sapiens |
| | | | | (ubiquinone) 1 alpha subcomplex, | |
| | | | | 8 (19 kD, PGIV) | |
| 1338 | AF044953_1 | 958 | 100 | AF044953 NADH: ubiquinone | Homo sapiens |
| | | | | oxidoreductase PGIV subunit | |
| 1338 | X59697 | 866 | 88 | 19 kDa subunit of NADH: ubiquinone | Bos taurus |
| | | | | oxidoreductase complex (complex | |
| | | | | I) | |
| 1339 | AAH01066 | 763 | 100 | BC001066 hypothetical protein | Homo sapiens |
| 1339 | AAH21986 | 763 | 100 | BC021986 mitochondrial ribosomal | Homo sapiens |
| | | | | protein L27 | |
| 1339 | AB049647 | 763 | 100 | mitochondrial ribosomal protein | Homo sapiens |
| | | | | L27 (L27mt) | |
| 1340 | AAH01101 | 2975 | 100 | BC001101 HSPC025 | Homo sapiens |
| 1340 | AAH07510 | 2975 | 100 | BC007510 HSPC025 | Homo sapiens |
| 1340 | BC029265 | 2975 | 100 | eukaryotic translation initiation | Homo sapiens |
| | | | | factor 3, subunit 6 interacting | |
| | | | | protein | |
| 1341 | AAH01150 | 2803 | 100 | BC001150 metalloprotease 1 | Homo sapiens |
| | | | | (pitrilysin family) | |
| 1341 | AAH05025 | 2802 | 99 | BC005025 Similar to | Homo sapiens |
| | | | | metalloprotease 1 (pitrilysin | |
| | | | | family) | |
| 1341 | AB029027 | 2803 | 100 | KIAA1104 protein | Homo sapiens |
| 1342 | AAH01288 | 2064 | 100 | BC001288 Similar to decay | Homo sapiens |
| | | | | accelerating factor for | |
| | | | | complement (CD55, Cromer blood | |
| | | | | group system) | |
| 1342 | M30142 | 2064 | 100 | decay-accelerating factor A | Homo sapiens |
| 1342 | M31516 | 2064 | 100 | decay-accelerating factor | Homo sapiens |
| 1343 | AAH01387 | 833 | 100 | BC001387 similar to rat HREV107 | Homo sapiens |
| 1343 | AB030814 | 833 | 100 | H-REV107 protein homolog | Homo sapiens |
| 1343 | AF317086 | 833 | 100 | HREV107-3 | Homo sapiens |
| 1344 | AAH01420 | 800 | 100 | BC001420 HN1 protein | Homo sapiens |
| 1344 | AF177862_1 | 800 | 100 | AF177862 HN1 protein | Homo sapiens |
| 1344 | AF348672 | 800 | 100 | hematological and neurological | Homo sapiens |
| | | | | expressed 1 protein | |
| 1345 | AAH01426 | 482 | 100 | BC001426 Similar to ubiquinol- | Homo sapiens |
| | | | | cytochrome c reductase hinge | |
| | | | | protein | |
| 1345 | AAH01934 | 482 | 100 | BC001934 Similar to ubiquinol- | Homo sapiens |
| | | | | cytochrome c reductase hinge | |
| | | | | protein | |
| 1345 | AAH15177 | 482 | 100 | BC015177 Unknown (protein for | Homo sapiens |
| | | | | MGC: 10149) | |
| 1346 | AAH21173 | 429 | 100 | BC021173 normal mucosa of | Homo sapiens |
| | | | | esophagus specific 1 | |
| 1346 | AB026707 | 429 | 100 | FOAP-11 protein | Homo sapiens |
| 1346 | AF228422_1 | 429 | 100 | AF228422 normal mucosa of | Homo sapiens |
| | | | | esophagus specific 1 | |
| 1347 | AF274958_1 | 737 | 100 | AF274958 PNAS-101 | Homo sapiens |
| 1348 | AAH14908 | 1453 | 100 | BC014908 stem-loop (histone) | Homo sapiens |
| | | | | binding protein | |
| 1348 | AAH15703 | 1453 | 100 | BC015703 stem-loop (histone) | Homo sapiens |
| | | | | binding protein | |
| 1348 | Z71188 | 1453 | 100 | histone RNA hairpin-binding | Homo sapiens |
| | | | | protein | |
| 1349 | AB000491 | 2046 | 100 | proteasome p45/SUG | Rattus |
| | | | | | norvegicus |
| 1349 | D83521 | 2046 | 100 | proteasomal ATPase (rat SUG1) | Rattus |
| | | | | | norvegicus |
| 1349 | Z54219 | 2046 | 100 | mSUG1 protein | Mus musculus |
| 1350 | AK075215 | 831 | 100 | unnamed protein product | Homo sapiens |
| 1351 | AAH02481 | 480 | 100 | BC002481 HSPC162 protein | Homo sapiens |
| 1351 | AF132750_1 | 480 | 100 | AF132750 bithoraxoid-like protein | Homo sapiens |
| 1351 | AF178431_1 | 480 | 100 | AF178431 BITH | Homo sapiens |
| 1352 | AAH02559 | 3087 | 100 | BC002559 high-glucose-regulated | Homo sapiens |
| | | | | protein 8 | |
| 1352 | AK083882 | 3077 | 99 | unnamed protein product | Mus musculus |
| 1352 | BC014797 | 3077 | 99 | Unknown (protein for MGC: 11691) | Mus musculus |
| 1353 | AAH02589 | 2230 | 100 | BC002589 proteasome (prosome, | Homo sapiens |
| | | | | macropain) 26S subunit, ATPase, 2 | |
| 1353 | BC005462 | 2226 | 99 | proteasome (prosome, macropain) | Mus musculus |
| | | | | 26S subunit, ATPase 2 | |
| 1353 | D11094 | 2230 | 100 | MSS1 protein | Homo sapiens |
| 1354 | AAH02634 | 1769 | 100 | BC002634 Unknown (protein for | Homo sapiens |
| | | | | MGC: 4272) | |
| 1354 | AK003354 | 1732 | 96 | unnamed protein product | Mus musculus |
| 1354 | BC002128 | 1725 | 96 | Unknown (protein for MGC: 6737) | Mus musculus |
| 1355 | AAH02803 | 843 | 100 | BC002803 hypothetical protein | Homo sapiens |
| 1355 | AF151072_1 | 843 | 100 | AF151072 HSPC238 | Homo sapiens |
| 1355 | BC005559 | 711 | 78 | RIKEN cDNA 2500002L14 gene | Mus musculus |
| 1356 | AAH02911 | 856 | 100 | BC002911 Unknown (protein for | Homo sapiens |
| | | | | MGC: 11276) | |
| 1356 | AK003237 | 722 | 84 | unnamed protein product | Mus musculus |
| 1356 | BC031732 | 722 | 84 | DNA segment, Chr 7, Wayne State | Mus musculus |
| | | | | University 86, expressed | |
| 1357 | AAH02945 | 2197 | 100 | BC002945 Similar to hypothetical | Homo sapiens |
| | | | | protein FLJ10101 | |
| 1357 | AAH21095 | 1290 | 100 | BC021095 Unknown (protein for | Homo sapiens |
| | | | | MGC: 31800) | |
| 1357 | AK027586 | 1290 | 100 | unnamed protein product | Homo sapiens |
| 1358 | AAH02954 | 2552 | 100 | BC002954 UDP-glucose | Homo sapiens |
| | | | | pyrophosphorylase 2 | |
| 1358 | BC023810 | 2524 | 98 | UDP-glucose pyrophosphorylase 2 | Mus musculus |
| 1358 | U27460 | 2542 | 99 | uridine diphosphoglucose | Homo sapiens |
| | | | | pyrophosphorylase | |
| 1359 | AAH03005 | 886 | 100 | BC003005 unactive progesterone | Homo sapiens |
| | | | | receptor, 23 kD | |
| 1359 | BC003708 | 876 | 98 | telomerase binding protein, p23 | Mus musculus |
| 1359 | L24804 | 886 | 100 | p23 | Homo sapiens |
| 1360 | AAH03056 | 2132 | 100 | BC003056 HSPC028 protein | Homo sapiens |
| 1360 | AAH08453 | 2132 | 100 | BC008453 HSPC028 protein | Homo sapiens |
| 1360 | AAH09597 | 2132 | 100 | BC009597 HSPC028 protein | Homo sapiens |
| 1361 | L29075 | 151 | 24 | G-box binding factor | Dictyostelium |
| | | | | | discoideum |
| 1362 | AAH01773 | 591 | 100 | BC001773 Similar to ribosomal | Homo sapiens |
| | | | | protein L34 | |
| 1362 | AB061832 | 591 | 100 | ribosomal protein L34 | Homo sapiens |
| 1362 | BC028517 | 587 | 99 | Unknown (protein for MGC: 41239) | Mus musculus |
| 1363 | AAH01882 | 533 | 100 | BC001882 Similar to ribosomal | Homo sapiens |
| | | | | protein L5 | |
| 1363 | D10737 | 512 | 100 | ribosomal protein L5 | Gallus |
| | | | | | gallus |
| 1363 | X57016 | 512 | 100 | ribosomal protein L5 | Gallus |
| | | | | | gallus |
| 1364 | AAH01884 | 589 | 100 | BC001884 NADH dehydrogenase | Homo sapiens |
| | | | | (ubiquinone) Fe-S protein 5 | |
| | | | | (15 kD) (NADH-coenzyme Q | |
| | | | | reductase) | |
| 1364 | AF020352 | 589 | 100 | NADH: ubiquinone oxidoreductase 15 kDa | Homo sapiens |
| | | | | IP subunit | |
| 1364 | AF047434 | 589 | 100 | NADH-ubiquinone oxidoreductase | Homo sapiens |
| | | | | 15 kDa subunit; CI-15 protein | |
| 1365 | AAH01926 | 2197 | 100 | BC001926 creatine kinase, | Homo sapiens |
| | | | | mitochondrial 1 (ubiquitous) | |
| 1365 | AAH06467 | 2197 | 100 | BC006467 creatine kinase, | Homo sapiens |
| | | | | mitochondrial 1 (ubiquitous) | |
| 1365 | J04469 | 2197 | 100 | creatine kinase | Homo sapiens |
| 1366 | AAH03373 | 763 | 100 | BC003373 prefoldin 5 | Homo sapiens |
| 1366 | AB055803 | 763 | 100 | MM-1 alpha | Homo sapiens |
| 1366 | D89667 | 763 | 100 | c-myc binding protein | Homo sapiens |
| 1367 | AAH05939 | 998 | 100 | BC005939 prostaglandin D2 | Homo sapiens |
| | | | | synthase (21 kD, brain) | |
| 1367 | AY026356 | 998 | 100 | prostaglandin D synthase | Homo sapiens |
| 1367 | M98539 | 998 | 100 | prostaglandin D2 synthase | Homo sapiens |
| 1368 | AAH00045 | 1388 | 100 | BC000045 TONDU | Homo sapiens |
| 1368 | AAH03362 | 1388 | 100 | BC003362 TONDU | Homo sapiens |
| 1368 | Z97632 | 1388 | 100 | dJ196E23.1.1 (novel protein) | Homo sapiens |
| | | | | (isoform 1) | |
| 1369 | AAH03366 | 796 | 100 | BC003366 calcium-regulated heat- | Homo sapiens |
| | | | | stable protein (24 kD) | |
| 1369 | AF115345_1 | 787 | 99 | AF115345 calcium-regulated heat | Homo sapiens |
| | | | | stable protein CRHSP-24 | |
| 1369 | AF115346_1 | 785 | 97 | AF115346 calcium-regulated heat | Rattus sp. |
| | | | | stable protein CRHSP-24 | |
| 1370 | AAH03369 | 581 | 100 | BC003369 ribosomal protein, | Homo sapiens |
| | | | | large, P1 | |
| 1370 | AAH07590 | 581 | 100 | BC007590 ribosomal protein, | Homo sapiens |
| | | | | large, P1 | |
| 1370 | AB061836 | 581 | 100 | ribosomal protein P1 | Homo sapiens |
| 1371 | AAH03377 | 550 | 100 | BC003377 Similar to thioredoxin | Homo sapiens |
| 1371 | AF313911_1 | 550 | 100 | AF313911 thioredoxin | Homo sapiens |
| 1371 | AY004872 | 550 | 100 | thioredoxin | Homo sapiens |
| 1372 | BC032493 | 1086 | 100 | cysteine and glycine-rich protein 1 | Homo sapiens |
| 1372 | M33146 | 1086 | 100 | cysteine-rich protein | Homo sapiens |
| 1372 | M76378 | 1086 | 100 | cysteine-rich protein | Homo sapiens |
| 1373 | AAH03382 | 2620 | 100 | BC003382 sorting nexin 2 | Homo sapiens |
| 1373 | AF065482 | 2612 | 99 | sorting nexin 2 | Homo sapiens |
| 1373 | AK075929 | 2564 | 98 | unnamed protein product | Mus musculus |
| 1374 | AAH03394 | 1485 | 100 | BC003394 heterogeneous nuclear | Homo sapiens |
| | | | | ribonucleoprotein C (C1/C2) | |
| 1374 | AAH08364 | 1478 | 99 | BC008364 heterogeneous nuclear | Homo sapiens |
| | | | | ribonucleoprotein C (C1/C2) | |
| 1374 | AAH08423 | 1461 | 95 | BC008423 heterogeneous nuclear | Homo sapiens |
| | | | | ribonucleoprotein C (C1/C2) | |
| 1375 | AAH03501 | 2155 | 100 | BC003501 Similar to RIKEN cDNA | Homo sapiens |
| | | | | 2310001A20 gene | |
| 1375 | AB033767 | 2155 | 100 | brain-selective and closely | Homo sapiens |
| | | | | mapped on the counter allele of | |
| | | | | CMAP in cystatin cluster | |
| 1375 | AL035661 | 2155 | 100 | dJ568C11.2 (chromosome 20 open | Homo sapiens |
| | | | | reading frame 3) | |
| 1376 | AAH03512 | 3204 | 100 | BC003512 mesothelin | Homo sapiens |
| 1376 | AE006464_21 | 3189 | 99 | AE006464 pre-pro-megakarycyte | Homo sapiens |
| | | | | potentiating factor precursor | |
| 1376 | D49441 | 3192 | 99 | pre-pro-megakaryocyte | Homo sapiens |
| | | | | potentiating factor | |
| 1377 | AAH03077 | 5288 | 100 | BC003077 Similar to ATPase, Na+K+ | Homo sapiens |
| | | | | transporting, alpha 1 polypeptide | |
| 1377 | D00099 | 5288 | 100 | Na, K-ATPase alpha-subunit | Homo sapiens |
| 1377 | X04297 | 5288 | 100 | ATPase alpha subunit (aa 1-1023) | Homo sapiens |
| 1378 | AAH03079 | 786 | 100 | BC003079 16.7 Kd protein | Homo sapiens |
| 1378 | AAH15639 | 786 | 100 | BC015639 16.7 Kd protein | Homo sapiens |
| 1378 | AF078845 | 786 | 100 | 16.7 Kd protein | Homo sapiens |
| 1379 | AAH00161 | 1827 | 100 | BC000161 secretory carrier | Homo sapiens |
| | | | | membrane protein 3 | |
| 1379 | AAH05135 | 1827 | 100 | BC005135 secretory carrier | Homo sapiens |
| | | | | membrane protein 3 | |
| 1379 | AF005039 | 1817 | 99 | secretory carrier membrane | Homo sapiens |
| | | | | protein | |
| 1380 | AAH08704 | 887 | 100 | BC008704 cytochrome c oxidase | Homo sapiens |
| | | | | subunit IV | |
| 1380 | AAH21236 | 887 | 100 | BC021236 cytochrome c oxidase | Homo sapiens |
| | | | | subunit IV isoform 1 | |
| 1380 | X54802 | 887 | 100 | cytochrome-c oxidase subunit IV | Homo sapiens |
| 1381 | AAH00915 | 1731 | 100 | BC000915 PDZ and LIM domain 1 | Homo sapiens |
| | | | | (elfin) | |
| 1381 | AAH18755 | 1731 | 100 | BC018755 PDZ and LIM domain 1 | Homo sapiens |
| | | | | (elfin) | |
| 1381 | AJ310549 | 1731 | 100 | CLP-36 protein | Homo sapiens |
| 1382 | AF236636 | 1302 | 98 | uridine-cytidine kinase 2 | Mus musculus |
| 1382 | AF236637 | 1333 | 100 | uridine-cytidine kinase 2 | Homo sapiens |
| 1382 | BC023789 | 1302 | 98 | uridine-cytidine kinase 2 | Mus musculus |
| 1383 | AAH00176 | 940 | 100 | BC000176 RAP1B, member of RAS | Homo sapiens |
| | | | | oncogene family | |
| 1383 | AF493913_1 | 940 | 100 | AF493913 Ras family small GTP | Homo sapiens |
| | | | | binding protein RAP1B | |
| 1383 | BC033382 | 1016 | 91 | RAP1B, member of RAS oncogene | Mus musculus |
| | | | | family | |
| 1384 | D63519 | 870 | 100 | leptin | Homo sapiens |
| 1384 | D63710 | 870 | 100 | ob protein | Homo sapiens |
| 1384 | U43653 | 870 | 100 | obese protein | Homo sapiens |
| 1385 | BC002088 | 637 | 100 | ribosomal protein S25 | Mus musculus |
| 1385 | BC027208 | 637 | 100 | ribosomal protein S25 | Mus musculus |
| 1385 | X62482 | 637 | 100 | ribosomal protein S25 | Rattus |
| | | | | | Rattus |
| 1386 | AAH03662 | 2092 | 100 | BC003662 KIAA0111 gene product | Homo sapiens |
| 1386 | AAH04386 | 2092 | 100 | BC004386 KIAA0111 gene product | Homo sapiens |
| 1386 | AAH11151 | 2092 | 100 | BC011151 Similar to KIAA0111 gene | Homo sapiens |
| | | | | product | |
| 1387 | AF349038_1 | 1780 | 100 | AF349038 TFIID subunit TAFII55 | Homo sapiens |
| 1387 | BC032737 | 1780 | 100 | similar to TAF7 RNA polymerase | Homo sapiens |
| | | | | II, TATA box binding protein | |
| | | | | (TBP)-associated factor, 55 kD | |
| 1387 | X97999 | 1780 | 100 | transcription factor IID | Homo sapiens |
| 1388 | AAH02601 | 1673 | 100 | BC002601 nuclear factor of kappa | Hom sapiens |
| | | | | light polypeptide gene enhancer | |
| | | | | in B-cells inhibitor, alpha | |
| 1388 | AAH04983 | 1673 | 100 | BC004983 nuclear factor of kappa | Homo sapiens |
| | | | | light polypeptide gene enhancer | |
| | | | | in B-cells inhibitor, alpha | |
| 1388 | AY033600 | 1673 | 100 | NFKBIA | Homo sapiens |
| 1389 | AF211480_1 | 2102 | 100 | AF211480 CD001 | Homo sapiens |
| 1389 | AK002102 | 2086 | 100 | unnamed protein product | Homo sapiens |
| 1389 | AX191505 | 2086 | 100 | unnamed protein product | Homo sapiens |
| 1390 | AAH05110 | 4609 | 100 | BC005110 Unknown (protein for | Homo sapiens |
| | | | | MGC: 13217) | |
| 1390 | AK049371 | 4473 | 93 | unnamed protein product | Mus musculus |
| 1390 | AY044865 | 4609 | 100 | sorting nexin 14 | Homo sapiens |
| 1391 | AAH05118 | 592 | 100 | BC005118 Similar to putative | Homo sapiens |
| | | | | translation initiation factor | |
| 1391 | AAH08710 | 592 | 100 | BC008710 putative translation | Homo sapiens |
| | | | | initiation factor | |
| 1391 | AF083441_1 | 592 | 100 | AF083441 SUI1 isolog | Homo sapiens |
| 1392 | AAH05127 | 2190 | 100 | BC005127 adipose differentiation- | Homo sapiens |
| | | | | related protein | |
| 1392 | AF443203_1 | 2190 | 100 | AF443203 adipose differentiation- | Homo sapiens |
| | | | | related protein | |
| 1392 | AX025098 | 2181 | 99 | unnamed protein product | Homo sapiens |
| 1393 | AAH05143 | 964 | 100 | BC005143 vitamin A responsive; | Homo sapiens |
| | | | | cytoskeleton related | |
| 1393 | AAH20797 | 964 | 100 | BC020797 vitamin A responsive; | Homo sapiens |
| | | | | cytoskeleton related | |
| 1393 | AY102608 | 964 | 100 | JWA protein | Homo sapiens |
| 1394 | AAH05228 | 360 | 100 | BC005228 Unknown (protein for | Homo sapiens |
| | | | | MGC: 12250) | |
| 1395 | AF072506 | 2871 | 100 | envelope protein precursor | Homo sapiens |
| 1395 | AF208161 | 2868 | 99 | syncytin precursor | Homo sapiens |
| 1395 | AF513360_1 | 2868 | 99 | AF513360 enverin | Homo sapiens |
| 1396 | AAH05322 | 1866 | 100 | BC005322 decorin | Homo sapiens |
| 1396 | AF138300 | 1866 | 100 | decorin variant A | Homo sapiens |
| 1396 | AF491944_1 | 1866 | 100 | AF491944 decorin | Homo sapiens |
| 1397 | AAH01392 | 827 | 100 | BC001392 ribosomal protein S27a | Homo sapiens |
| 1397 | D83209 | 827 | 100 | ubiquitin extention protein | Cavia |
| | | | | | porcellus |
| 1397 | M24507 | 827 | 100 | ubiquitin | synthetic |
| | | | | | construct |
| 1398 | AAH05330 | 1306 | 100 | BC005330 tissue factor pathway | Homo sapiens |
| | | | | inhibitor 2 | |
| 1398 | AF217542 | 1306 | 100 | tissue factor pathway inhibitor 2 | Homo sapiens |
| 1398 | D29992 | 1306 | 100 | placental protein 5 (PP5) | Homo sapiens |
| 1399 | AAH05361 | 1334 | 100 | BC005361 proteasome (prosome, | Homo sapiens |
| | | | | macropain) subunit, alpha type, 4 | |
| 1399 | BC022445 | 1334 | 100 | proteasome (prosome, macropain) | Homo sapiens |
| | | | | subunit, alpha type, 4 | |
| 1399 | D00763 | 1334 | 100 | proteasome subunit C9 | Homo sapiens |
| 1400 | AAH05366 | 1298 | 100 | BC005366 ATP synthase, H+ | Homo sapiens |
| | | | | transporting, mitochondrial F0 | |
| | | | | complex, subunit b, isoform 1 | |
| 1400 | AAH16350 | 1298 | 100 | BC016350 Unknown (protein for | Homo sapiens |
| | | | | MGC: 24431) | |
| 1400 | AL390195 | 1298 | 100 | bA552M11.3 (ATP synthase, H+ | Homo sapiens |
| | | | | transporting, mitochondrial F0 | |
| | | | | complex, subunit b, isoform 1) | |
| 1401 | AAH05390 | 1993 | 100 | BC005390 Unknown (protein for | Homo sapiens |
| | | | | MGC: 12520) | |
| 1401 | AF006305 | 1993 | 100 | 26S proteasome regulatory subunit | Homo sapiens |
| 1401 | U36395 | 1993 | 100 | conserved ATPase domain protein | Spermophilus |
| | | | | 44 | tridecemlineatus |
| 1402 | AAH10370 | 1800 | 99 | BC010370 Putative prostate cancer | Homo sapiens |
| | | | | tumor suppressor | |
| 1402 | U42349 | 1826 | 100 | 39 kDa encoded by N33 | Homo sapiens |
| 1402 | U42360 | 1808 | 99 | N33 protein form 1 | Homo sapiens |
| 1403 | AAH05839 | 1492 | 100 | BC005839 follistatin-like 3 | Homo sapiens |
| | | | | (secreted glycoprotein) | |
| 1403 | BC033119 | 1492 | 100 | follistatin-like 3 (secreted | Homo sapiens |
| | | | | glycoprotein) | |
| 1403 | U76702 | 1492 | 100 | follistatin-related protein FLRG | Homo sapiens |
| 1404 | AAH05354 | 564 | 100 | BC005354 ribosomal protein, large | Homo sapiens |
| | | | | P2 | |
| 1404 | AAH05920 | 564 | 100 | BC005920 ribosomal protein, large | Homo sapiens |
| | | | | P2 | |
| 1404 | AAH07573 | 564 | 100 | BC007573 ribosomal protein, large | Homo sapiens |
| | | | | P2 | |
| 1405 | AAH17660 | 414 | 100 | BC017660 Unknown (protein for | Homo sapiens |
| | | | | MGC: 14608) | |
| 1405 | AJ249731 | 414 | 100 | putative G8.2 protein | Homo sapiens |
| 1405 | AP000503 | 414 | 100 | unknown function | Homo sapiens |
| 1406 | AAH05966 | 647 | 100 | BC005966 ring finger protein 7 | Homo sapiens |
| 1406 | AAH08627 | 647 | 100 | BC008627 ring finger protein 7 | Homo sapiens |
| 1406 | AF092878_1 | 647 | 100 | AF092878 zinc RING finger protein | Homo sapiens |
| | | | | SAG | |
| 1407 | AAH05975 | 1163 | 100 | BC005975 calcyclin binding | Homo sapiens |
| | | | | protein | |
| 1407 | AF314752_1 | 1163 | 100 | AF314752 calcyclin binding | Homo sapiens |
| | | | | protein | |
| 1407 | BC022352 | 1163 | 100 | Siah-interacting protein | Homo sapiens |
| 1408 | AAH06337 | 739 | 100 | BC006337 Unknown (protein for | Homo sapiens |
| | | | | MGC: 12798) | |
| 1408 | M37194 | 739 | 100 | clathrin-associated protein 17 | Rattus |
| | | | | | norvegicus |
| 1408 | X97074 | 729 | 98 | clathrin-associated protein | Homo sapiens |
| 1409 | AAH01928 | 3365 | 100 | BC001928 protein disulfide | Homo sapiens |
| | | | | isomerase related protein | |
| | | | | (calcium-binding protein, | |
| | | | | intestinal-related) | |
| 1409 | AAH06344 | 3365 | 100 | BC006344 protein disulfide | Homo sapiens |
| | | | | isomerase related protein | |
| | | | | (calcium-binding protein, | |
| | | | | intestinal-related) | |
| 1409 | AAH11754 | 3365 | 100 | BC011754 Similar to protein | Homo sapiens |
| | | | | disulfide isomerase related | |
| | | | | protein (calcium-binding protein, | |
| | | | | intestinal-related) | |
| 1410 | AF453478_1 | 1155 | 100 | AF453478 phosphopantetheine | Homo sapiens |
| | | | | adenylyltransferase/ | |
| | | | | dephosphocoenzyme A kinase | |
| 1410 | AY094602 | 1155 | 100 | bifunctional phosphopantetheine | Homo sapiens |
| | | | | adenylyl transferase/dephospho | |
| | | | | CoA kinase | |
| 1410 | BC020985 | 1155 | 100 | Unknown (protein for MGC: 9724) | Homo sapiens |
| 1411 | AAH06393 | 2797 | 100 | BC006393 Similar to | Homo sapiens |
| | | | | carboxypeptidase Z | |
| 1411 | AF017638 | 2419 | 86 | carboxypeptidase Z | Rattus |
| | | | | | norvegicus |
| 1411 | U83411 | 2787 | 99 | carboxypeptidase Z precursor | Homo sapiens |
| 1412 | AF126110_1 | 3913 | 100 | AF126110 fibulin-1 isoform D | Homo sapiens |
| | | | | precursor | |
| 1412 | U01244 | 3913 | 100 | fibulin-1D | Homo sapiens |
| 1412 | X70854 | 3392 | 84 | BH-90/fibulin | Mus musculus |
| 1413 | AAH02343 | 2494 | 100 | BC002343 Similar to nucleolin | Homo sapiens |
| 1413 | AAH06494 | 2494 | 100 | BC006494 Unknown (protein for | Homo sapiens |
| | | | | MGC: 1440) | |
| 1413 | AAH06516 | 2494 | 100 | BC006516 Unknown (protein for | Homo sapiens |
| | | | | MGC: 3588) | |
| 1414 | AAH20515 | 772 | 100 | BC020515 ribosomal protein S14 | Homo sapiens |
| 1414 | M11241 | 772 | 100 | ribosomal protein S14 | Cricetulus |
| | | | | | griseus |
| 1414 | M35008 | 772 | 100 | ribosomal protein S14 | Cricetulus |
| | | | | | griseus |
| 1415 | AAH08926 | 796 | 98 | BC008926 ribosomal protein L29 | Homo sapiens |
| 1415 | U10248 | 796 | 98 | ribosomal protein L29 | Homo sapiens |
| 1415 | U49083 | 796 | 98 | HIP | Homo sapiens |
| 1416 | AAH06791 | 1105 | 100 | BC006791 ribosomal protein L10a | Homo sapiens |
| 1416 | AAH11366 | 1105 | 100 | BC011366 Similar to ribosomal | Homo sapiens |
| | | | | protein L10a | |
| 1416 | X93352 | 1105 | 100 | ribosomal protein L10a | Rattus |
| | | | | | norvegicus |
| 1417 | AAH01418 | 1132 | 100 | BC001418 Similar to RIKEN cDNA | Homo sapiens |
| | | | | 1810017F10 gene | |
| 1417 | AAH13953 | 1132 | 100 | BC013953 Unknown (protein for | Homo sapiens |
| | | | | MGC: 2853) | |
| 1417 | AY027543 | 1132 | 100 | beta-casein-like protein | Homo sapiens |
| 1418 | AAH07038 | 1762 | 100 | BC007038 lumican | Homo sapiens |
| 1418 | BC035997 | 1762 | 100 | lumican | Homo sapiens |
| 1418 | U21128 | 1762 | 100 | lumican | Homo sapiens |
| 1419 | AAH03609 | 1441 | 100 | BC003609 thioredoxin peroxidase | Homo sapiens |
| | | | | (antioxidant enzyme) | |
| 1419 | AAH07107 | 1441 | 100 | BC007107 thioredoxin peroxidase | Homo sapiens |
| | | | | (antioxidant enzyme) | |
| 1419 | AAH16770 | 1441 | 100 | BC016770 thioredoxin peroxidase | Homo sapiens |
| | | | | (antioxidant enzyme) | |
| 1420 | AAH07282 | 952 | 100 | BC007282 Unknown (protein for | Homo sapiens |
| | | | | MGC: 15626) | |
| 1420 | AK014338 | 909 | 97 | unnamed protein product | Mus musculus |
| 1420 | M83751 | 937 | 98 | arginine-rich protein | Homo sapiens |
| 1421 | AAH07402 | 1005 | 100 | BC007402 apolipoprotein D | Homo sapiens |
| 1421 | J02611 | 1005 | 100 | apolipoprotein D precursor | Homo sapiens |
| 1421 | M16696 | 1005 | 100 | apolipoprotein D precursor | Homo sapiens |
| 1422 | AAH01603 | 849 | 100 | BC001603 Similar to ribosomal | Homo sapiens |
| | | | | protein L21 | |
| 1422 | AAH07505 | 849 | 100 | BC007505 Unknown (protein for | Homo sapiens |
| | | | | MGC: 4136) | |
| 1422 | X89401 | 849 | 100 | ribosomal protein L21 | Homo sapiens |
| 1423 | AAH07507 | 605 | 100 | BC007507 ribosomal protein S20 | Homo sapiens |
| 1423 | BC011323 | 605 | 100 | Similar to ribosomal protein S20 | Mus musculus |
| 1423 | X51537 | 605 | 100 | ribosomal protein S20 (AA 1-119) | Rattus |
| | | | | | Rattus |
| 1424 | AAH14459 | 791 | 100 | BC014459 Similar to ribosomal | Homo sapiens |
| | | | | protein L23a | |
| 1424 | BC029892 | 791 | 100 | ribosomal protein L23a | Mus musculus |
| 1424 | X65228 | 791 | 100 | ribosomal protein L23a | Rattus |
| | | | | | Rattus |
| 1425 | AJ420896 | 2114 | 99 | SPPL2a protein | Homo sapiens |
| 1425 | AK027446 | 2126 | 100 | unnamed protein product | Homo sapiens |
| 1425 | BC025740 | 2114 | 99 | Similar to hypothetical protein | Homo sapiens |
| | | | | FLJ14540 | |
| 1426 | AF036548 | 573 | 83 | RGC-32 | Rattus |
| | | | | | norvegicus |
| 1426 | AF036549_1 | 569 | 100 | AF036549 RGC32 | Homo sapiens |
| 1426 | AL354833 | 629 | 92 | bA157L14.2 (RGC32, a novel gene | Homo sapiens |
| | | | | induced by complement activation | |
| | | | | in oligodendrocytes) | |
| 1427 | AAH01024 | 2749 | 99 | BC001024 putative nucleotide | Homo sapiens |
| | | | | binding protein, estradiol- | |
| | | | | induced | |
| 1427 | AK027514 | 2756 | 100 | unnamed protein product | Homo sapiens |
| 1427 | AK027516 | 2753 | 99 | unnamed protein product | Homo sapiens |
| 1428 | AAH07834 | 563 | 100 | BC007834 Unknown (protein for | Homo sapiens |
| | | | | MGC: 14141) | |
| 1429 | AAH03061 | 2338 | 99 | BC003061 protease, cysteine, 1 | Homo sapiens |
| | | | | (legumain) | |
| 1429 | D55696 | 2335 | 99 | cysteine protease | Homo sapiens |
| 1429 | Y09862 | 2339 | 100 | legumain | Homo sapiens |
| 1430 | AAH08081 | 1095 | 100 | BC008081 Similar to KDEL (Lys- | Homo sapiens |
| | | | | Asp-Glu-Leu) endoplasmic | |
| | | | | reticulum protein retention | |
| | | | | receptor 2 | |
| 1430 | AAH14568 | 1095 | 100 | BC014568 Similar to KDEL (Lys- | Homo sapiens |
| | | | | Asp-Glu-Leu) endoplasmic | |
| | | | | reticulum protein retention | |
| | | | | receptor 2 | |
| 1430 | X63745 | 1095 | 100 | KDEL receptor | Homo sapiens |
| 1431 | AF004877 | 7574 | 99 | pro-alpha 2(I) collagen | Homo sapiens |
| 1431 | J03464 | 7554 | 99 | pre-pro-alpha-2 type I collagen | Homo sapiens |
| 1431 | Z74616 | 7596 | 100 | prepro-alpha2(I) collagen | Homo sapiens |
| 1432 | AAH08188 | 946 | 100 | BC008188 Unknown (protein for | Homo sapiens |
| | | | | MGC: 5243) | |
| 1432 | AF021819 | 946 | 100 | RNA-binding protein regulatory | Homo sapiens |
| | | | | subunit | |
| 1432 | AL034417 | 946 | 100 | bK215D11.1 (RNA-binding protein | Homo sapiens |
| | | | | regulatory subunit) | |
| 1433 | AAH08283 | 594 | 100 | BC008283 cholecystokinin | Homo sapiens |
| 1433 | L00354 | 594 | 100 | cholecystokinin | Homo sapiens |
| 1433 | M60458 | 569 | 95 | cholecystokinin | Macaca |
| | | | | | fascicularis |
| 1434 | AAH06794 | 693 | 99 | BC006794 Similar to interferon | Homo sapiens |
| | | | | induced transmembrane protein 3 | |
| | | | | (1-8U) | |
| 1434 | AAH08417 | 698 | 100 | BC008417 Similar to interferon | Homo sapiens |
| | | | | induced transmembrane protein 3 | |
| | | | | (1-8U) | |
| 1434 | BC022439 | 698 | 100 | interferon induced transmembrane | Homo sapiens |
| | | | | protein 3 (1-8U) | |
| 1435 | AAH05993 | 1421 | 100 | BC005993 Unknown (protein for | Homo sapiens |
| | | | | MGC: 14796) | |
| 1435 | AAH08691 | 1421 | 100 | BC008691 Unknown (protein for | Homo sapiens |
| | | | | MGC: 8886) | |
| 1435 | AAH15644 | 1421 | 100 | BC015644 Ras suppressor protein 1 | Homo sapiens |
| 1436 | AAH08938 | 1942 | 100 | BC008938 Similar to | Homo sapiens |
| | | | | histocompatibility 13 | |
| 1436 | AAH08959 | 1942 | 100 | BC008959 Similar to | Homo sapiens |
| | | | | histocompatibility 13 | |
| 1436 | AF483215_1 | 1934 | 99 | AF483215 minor histocompatibility | Homo sapiens |
| | | | | antigen H13 isoform 1 | |
| 1437 | AAH16320 | 2150 | 100 | BC016320 cathepsin D (lysosomal | Homo sapiens |
| | | | | aspartyl protease) | |
| 1437 | M11233 | 2150 | 100 | preprocathepsin D | Homo sapiens |
| 1437 | X05344 | 2150 | 100 | precursor polypeptide (AA −20 to | Homo sapiens |
| | | | | 392) | |
| 1438 | AAH08983 | 1364 | 100 | BC008983 complement component 1, | Homo sapiens |
| | | | | q subcomponent, beta polypeptide | |
| 1438 | X03084 | 1240 | 100 | C1q B-chain precursor | Homo sapiens |
| 1438 | X16874 | 1075 | 79 | precursor polypeptide (AA −25 to | Mus musculus |
| | | | | 228) | |
| 1439 | AAH09177 | 1014 | 100 | BC009177 ras homolog gene family, | Homo sapiens |
| | | | | member C | |
| 1439 | AF498972_1 | 1014 | 100 | AF498972 small GTP binding | Homo sapiens |
| | | | | protein RhoC | |
| 1439 | BC004627 | 1014 | 100 | ras homolog gene family, member C | Mus musculus |
| 1440 | AAH09200 | 1068 | 100 | BC009200 Rho GDP dissociation | Homo sapiens |
| | | | | inhibitor (GDI) beta | |
| 1440 | AF498927_1 | 1068 | 100 | AF498927 Rho GDP dissociation | Homo sapiens |
| | | | | inhibitor beta | |
| 1440 | X69549 | 1068 | 100 | Human rho GDP-dissociation | Homo sapiens |
| | | | | Inhibitor 2(IEF 8120) | |
| 1441 | AAH09201 | 1137 | 100 | BC009201 clathrin, light | Homo sapiens |
| | | | | polypeptide (Lca) | |
| 1441 | AAH19287 | 1137 | 100 | BC019287 clathrin, light | Homo sapiens |
| | | | | polypeptide (Lca) | |
| 1441 | AL158830 | 1137 | 100 | bA421H8.4.2 (clathrin, light | Homo sapiens |
| | | | | polypeptide (LCA)) | |
| 1442 | AAH09232 | 596 | 100 | BC009232 Similar to G antigen 8 | Homo sapiens |
| 1442 | AJ318880 | 596 | 100 | XAGE-2 protein | Homo sapiens |
| 1442 | AJ318891 | 596 | 100 | XAGE-2 protein | Homo sapiens |
| 1443 | AAH03378 | 1149 | 100 | BC003378 high-mobility group | Homo sapiens |
| | | | | (nonhistone chromosomal) protein 1 | |
| 1443 | BC030981 | 1149 | 100 | high-mobility group (nonhistone | Homo sapiens |
| | | | | chromosomal) protein 1 | |
| 1443 | X12597 | 1149 | 100 | HMG-1 protein (AA 1-215) | Homo sapiens |
| 1444 | AB002368 | 5586 | 100 | KIAA0370 | Homo sapiens |
| 1444 | AY026388 | 5765 | 100 | ran binding protein RanBP20 | Homo sapiens |
| 1444 | AY029528 | 5663 | 97 | RANBP20 | Mus musculus |
| 1445 | AAH09799 | 1308 | 100 | BC009799 amphiregulin | Homo sapiens |
| | | | | (schwannoma-derived growth | |
| | | | | factor) | |
| 1445 | M30703 | 1308 | 100 | amphiregulin | Homo sapiens |
| 1445 | M30704 | 1308 | 100 | amphiregulin | Homo sapiens |
| 1446 | AAH09869 | 511 | 100 | BC009869 Unknown (protein for | Homo sapiens |
| | | | | MGC: 16406) | |
| 1446 | AAH15491 | 511 | 100 | BC015491 Unknown (protein for | Homo sapiens |
| | | | | MGC: 8965) | |
| 1446 | BC022326 | 511 | 100 | Unknown (protein for MGC: 22741) | Homo sapiens |
| 1447 | AAH09898 | 1921 | 100 | BC009898 spermine synthase | Homo sapiens |
| 1447 | AD001528 | 1921 | 100 | spermidine aminopropyltransferase | Homo sapiens |
| 1447 | Z49099 | 1916 | 100 | spermine synthase | Homo sapiens |
| 1448 | AL365410 | 1055 | 100 | hypothetical protein | Homo sapiens |
| 1449 | AAH10016 | 1542 | 100 | BC010016 Similar to CD47 antigen | Homo sapiens |
| | | | | (Rh-related antigen, integrin- | |
| | | | | associated signal transducer) | |
| 1449 | AAH12884 | 1542 | 100 | BC012884 Unknown (protein for | Homo sapiens |
| | | | | MGC: 9240) | |
| 1449 | Z25521 | 1542 | 100 | integrin associated protein | Homo sapiens |
| 1450 | AAH10055 | 784 | 100 | BC010055 hypothetical protein | Homo sapiens |
| | | | | PRO2605 | |
| 1450 | AF116721_109 | 642 | 100 | AF116709 PRO2605 | Homo sapiens |
| 1451 | AAH04976 | 634 | 100 | BC004976 Unknown (protein for | Homo sapiens |
| | | | | IMAGE: 2958115) | |
| 1451 | AAH10076 | 634 | 100 | BC010076 Unknown (protein for | Homo sapiens |
| | | | | MGC: 19576) | |
| 1451 | AY061855 | 634 | 100 | mitochondrial ribosomal protein | Homo sapiens |
| | | | | S6 | |
| 1452 | AAH10129 | 907 | 100 | BC010129 Similar to hypothetical | Homo sapiens |
| | | | | protein R33729_1 | |
| 1452 | AAH14655 | 907 | 100 | BC014655 Unknown (protein for | Homo sapiens |
| | | | | MGC: 20383) | |
| 1452 | AL365374 | 894 | 100 | R33729_1 hypothetical protein | Homo sapiens |
| 1453 | AB028894 | 824 | 100 | ribosomal protein S11 | Mus musculus |
| 1453 | BC012641 | 824 | 100 | ribosomal protein S11 | Mus musculus |
| 1453 | U93864 | 824 | 100 | ribosomal protein S11 | Mus musculus |
| 1454 | BC002077 | 1058 | 100 | RAB1, member RAS oncogene family | Mus musculus |
| 1454 | X15744 | 1058 | 100 | GTP-binding protein | Mus musculus |
| 1454 | Y00094 | 1058 | 100 | Ypt1 protein (AA 1-205) | Mus musculus |
| 1455 | AAH02947 | 1482 | 100 | BC002947 folate receptor 1 | Homo sapiens |
| | | | | (adult) | |
| 1455 | U20391 | 1482 | 100 | folate receptor | Homo sapiens |
| 1455 | X62753 | 1482 | 100 | adult folate binding protein | Homo sapiens |
| 1456 | U65932 | 3016 | 100 | extracellular matrix protein 1 | Homo sapiens |
| 1456 | U65938 | 3016 | 100 | extracellular matrix protein 1 | Homo sapiens |
| 1456 | U68186 | 3016 | 100 | extracellular matrix protein 1 | Homo sapiens |
| 1457 | AAH10897 | 541 | 100 | BC010897 Similar to JM27 protein | Homo sapiens |
| 1457 | AF275258_1 | 541 | 100 | AF275258 PAGE-4 | Homo sapiens |
| 1457 | AJ005894 | 541 | 100 | JM27 | Homo sapiens |
| 1458 | AAH11171 | 2528 | 100 | BC011171 serine (or cysteine) | Homo sapiens |
| | | | | proteinase inhibitor, clade G (C1 | |
| | | | | inhibitor), member 1 | |
| 1458 | AF435921_1 | 2524 | 99 | AF435921 C1 esterase inhibitor | Homo sapiens |
| 1458 | X07427 | 2524 | 99 | C1 inhibitor | Homo sapiens |
| 1459 | D38112 | 1803 | 93 | cytochrome b | Homo sapiens |
| 1459 | U09500 | 1802 | 93 | cytochrome b | Homo sapiens |
| 1459 | V00662 | 1807 | 93 | cytochrome B | Homo sapiens |
| 1460 | AAH11514 | 696 | 100 | BC011514 Similar to ribosomal | Homo sapiens |
| | | | | protein L32 | |
| 1460 | K02060 | 696 | 100 | ribosomal protein L32-3A | Mus musculus |
| 1460 | X06483 | 696 | 100 | ribosomal protein L32 | Rattus |
| | | | | | norvegicus |
| 1461 | AAH11786 | 645 | 100 | BC011786 Unknown (protein for | Homo sapiens |
| | | | | MGC: 19839) | |
| 1461 | AF318382_1 | 645 | 100 | AF318382 unknown | Homo sapiens |
| 1461 | X07868 | 456 | 100 | 1.8 kb mRNA (AA 1-84) | Homo sapiens |
| 1462 | AAH11792 | 1649 | 100 | BC011792 Unknown (protein for | Homo sapiens |
| | | | | MGC: 19561) | |
| 1462 | AAH17408 | 1643 | 99 | BC017408 Unknown (protein for | Homo sapiens |
| | | | | MGC: 27221) | |
| 1462 | U16660 | 1603 | 97 | peroxisomal enoyl-CoA hydratase- | Homo sapiens |
| | | | | like protein | |
| 1463 | AAH00159 | 2163 | 100 | BC000159 keratin 17 | Homo sapiens |
| 1463 | AAH11901 | 2163 | 100 | BC01901 Similar to keratin 17 | Homo sapiens |
| 1463 | X62571 | 2163 | 100 | keratin related product | Homo sapiens |
| 1464 | AAH12132 | 3933 | 100 | BC012132 Similar to DEAD/H (Asp- | Homo sapiens |
| | | | | Glu-Ala-Asp/His) box polypeptide 1 | |
| 1464 | BC010624 | 3872 | 97 | Unknown (protein for MGC: 11570) | Mus musculus |
| 1464 | X70649 | 3933 | 100 | member of DEAD box protein family | Homo sapiens |
| 1465 | AF157623_1 | 2466 | 100 | AF157623 HTRA serine protease | Homo sapiens |
| 1465 | D87258 | 2466 | 100 | serin protease with IGF-binding | Homo sapiens |
| | | | | motif | |
| 1465 | Y07921 | 2466 | 100 | novel serine protease, PRSS11 | Homo sapiens |
| 1466 | AF242550_1 | 1030 | 100 | AF242550 cellular nucleic acid | Rattus |
| | | | | binding protein | norvegicus |
| 1466 | AF389887 | 1030 | 100 | zinc finger protein 9 | Homo sapiens |
| 1466 | D45254 | 1030 | 100 | Cellular Nucleic Acid Binding | Rattus |
| | | | | Protein | norvegicus |
| 1467 | AAH14277 | 1191 | 100 | BC014277 Similar to tissue | Homo sapiens |
| | | | | inhibitor of metalloproteinase 3 | |
| | | | | (Sorsby fundus dystrophy, | |
| | | | | pseudoinflammatory) | |
| 1467 | U33114 | 1191 | 100 | tissue inhibitor of | Homo sapiens |
| | | | | metalloproteinases-3 | |
| 1467 | X76227 | 1191 | 100 | tissue inhibitor of | Homo sapiens |
| | | | | metalloproteinases-3 | |
| 1468 | AAH12384 | 1017 | 100 | BC012384 Similar to programmed | Homo sapiens |
| | | | | cell death 6 | |
| 1468 | AF035606 | 1017 | 100 | calcium binding protein | Homo sapiens |
| 1468 | U58773_1 | 1017 | 100 | U58773 calcium binding protein | Homo sapiens |
| 1469 | AAH12464 | 755 | 100 | BC012464 prefoldin 2 | Homo sapiens |
| 1469 | AF117237_1 | 755 | 100 | AF117237 prefoldin subunit 2 | Homo sapiens |
| 1469 | BC026839 | 729 | 96 | Similar to prefoldin 2 | Mus musculus |
| 1470 | AAH08230 | 849 | 100 | BC008230 ribosomal protein L12 | Homo sapiens |
| 1470 | BC018321 | 848 | 99 | ribosomal protein L12 | Mus musculus |
| 1470 | L06505 | 849 | 100 | ribosomal protein L12 | Homo sapiens |
| 1471 | AAH12606 | 1665 | 100 | BC012606 Similar to proteasome | Homo sapiens |
| | | | | (prosome, macropain) 26S subunit, | |
| | | | | non-ATPase, 7 (Mov34 homolog) | |
| 1471 | D50063 | 1637 | 98 | proteasome subunit p40/Mov34 | Homo sapiens |
| | | | | protein | |
| 1471 | M64640 | 1597 | 96 | 36 kD protein | Mus musculus |
| 1472 | AAH11835 | 1485 | 100 | BC011835 Similar to ATPase, | Homo sapiens |
| | | | | Na+/K+ transporting, beta 3 | |
| | | | | polypeptide | |
| 1472 | AF005896 | 1485 | 100 | Na K-ATPase beta-3 subunit | Homo sapiens |
| 1472 | U51478 | 1485 | 100 | sodium/potassium-transporting | Homo sapiens |
| | | | | ATPase beta-3 subunit | |
| 1473 | AAH13041 | 5526 | 100 | BC013041 Unknown (protein for | Homo sapiens |
| | | | | MGC: 4781) | |
| 1473 | M58028 | 5526 | 100 | ubiquitin-activating enzyme E1 | Homo sapiens |
| 1473 | X56976 | 5516 | 99 | ubiquitin activating enzyme E1 | Homo sapiens |
| 1474 | AAH13162 | 491 | 100 | BC013162 Similar to heat shock | Homo sapiens |
| | | | | protein, 30 kDa | |
| 1474 | AF085359 | 491 | 100 | HSPC030 | Homo sapiens |
| 1474 | AF537132 | 491 | 100 | selenoprotein K | Homo sapiens |
| 1475 | AAH13175 | 992 | 100 | BC013175 Similar to novel RGD- | Homo sapiens |
| | | | | containing protein | |
| 1475 | BC029249 | 958 | 96 | dynactin 6 | Mus musculus |
| 1475 | D84145 | 992 | 100 | WS-3 | Homo sapiens |
| 1476 | BC003825 | 2344 | 100 | tubulin, beta 5 | Mus musculus |
| 1476 | X04663 | 2344 | 100 | beta-tubulin AA 1-444 (79 is 1st | Mus musculus |
| | | | | base in codon) | |
| 1476 | X07011 | 2344 | 100 | c(beta)7 tubulin (AA 1-444) | Gallus |
| | | | | | gallus |
| 1477 | A47413 | 3336 | 96 | CALPASTATINE HUMAINE | Homo sapiens |
| 1477 | AAH13579 | 3420 | 100 | BC013579 Similar to calpastatin | Homo sapiens |
| 1477 | D16217 | 3340 | 93 | calpastatin | Homo sapiens |
| 1478 | BC024378 | 567 | 100 | defender against cell death 1 | Mus musculus |
| 1478 | Y13335 | 567 | 100 | DAD-1 | Mus musculus |
| 1478 | Y13336 | 567 | 100 | DAD-1 | Rattus |
| | | | | | norvegicus |
| 1479 | AAH13975 | 3963 | 100 | BC013975 Similar to sema domain, | Homo sapiens |
| | | | | immunoglobulin domain (Ig), short | |
| | | | | basic domain, secreted, | |
| | | | | (semaphorin) 3B | |
| 1479 | BC024220 | 3963 | 100 | Unknown (protein for MGC: 1483) | Homo sapiens |
| 1479 | U28369 | 3951 | 99 | semaphorin V | Homo sapiens |
| 1480 | AAH14142 | 3503 | 100 | BC014142 Similar to | Homo sapiens |
| | | | | glucuronidase, beta | |
| 1480 | AX147652 | 3496 | 99 | unnamed protein product | Homo sapiens |
| 1480 | M15182 | 3496 | 99 | beta-glucuronidase precursor (EC | Homo sapiens |
| | | | | 3.2.1.31) | |
| 1481 | AB022163 | 2024 | 92 | mouse ortholog of the zebrafish | Mus musculus |
| | | | | hagoromo gene | |
| 1481 | AF281859 | 2217 | 100 | dactylin | Homo sapiens |
| 1481 | BC027031 | 2024 | 92 | f-box and WD-40 domain protein 4 | Mus musculus |
| 1482 | AAH01377 | 1114 | 100 | BC001377 DKFZP586G1722 protein | Homo sapiens |
| 1482 | AAH06514 | 1114 | 100 | BC006514 DKFZP586G1722 protein | Homo sapiens |
| 1482 | AAH14391 | 1114 | 100 | BC014391 DKFZP586G1722 protein | Homo sapiens |
| 1483 | AF159256_1 | 1147 | 100 | AF159256 Lps/Ran GTPase | Mus musculus |
| 1483 | AF306457_1 | 1147 | 100 | AF306457 GTPase | Rattus |
| | | | | | norvegicus |
| 1483 | BC014829 | 1147 | 100 | RAN, member RAS oncogene family | Mus musculus |
| 1484 | AAH15000 | 1347 | 100 | BC015000 Similar to major | Homo sapiens |
| | | | | histocompatibility complex, class | |
| | | | | II, DP beta 1 | |
| 1484 | M57466 | 1306 | 96 | light chain | Homo sapiens |
| 1484 | X01426 | 1301 | 96 | SB-2-beta precursor polypeptide | Homo sapiens |
| | | | | (aa −29 to 229) | |
| 1485 | AAH15039 | 1006 | 100 | BC015039 microfibrillar- | Homo sapiens |
| | | | | associated protein 2 | |
| 1485 | AL049569 | 1006 | 100 | dJ37C10.4 (microfibrillar- | Homo sapiens |
| | | | | associated protein 2 | |
| | | | | (microfibril-associated | |
| | | | | glycoprotein precursor, MGAP1)) | |
| 1485 | U19718 | 1006 | 100 | microfibril-associated | Homo sapiens |
| | | | | glycoprotein | |
| 1486 | AF117383_1 | 746 | 100 | AF117383 placental protein 13; | Homo sapiens |
| | | | | PP13 | |
| 1486 | AY055826 | 746 | 100 | placenta protein 13 | Homo sapiens |
| 1486 | BC022257 | 510 | 69 | Similar to placental protein 13- | Homo sapiens |
| | | | | like protein | |
| 1487 | AAH02387 | 2063 | 100 | BC002387 nucleosome assembly | Homo sapiens |
| | | | | protein 1-like 1 | |
| 1487 | AAH15599 | 2063 | 100 | BC015599 nucleosome assembly | Homo sapiens |
| | | | | protein 1-like 1 | |
| 1487 | AL162068 | 2063 | 100 | hypothetical protein | Homo sapiens |
| 1488 | A03911 | 2017 | 99 | glia-derived neurite-promoting | Homo sapiens |
| | | | | factor (GdNPF) | |
| 1488 | AAH15663 | 2040 | 100 | BC015663 Similar to serine (or | Homo sapiens |
| | | | | cysteine) proteinase inhibitor, | |
| | | | | clade E (nexin, plasminogen | |
| | | | | activator inhibitor type 1) | |
| | | | | member 2 | |
| 1488 | M17783 | 2016 | 99 | glia-derived nexin precursor | Homo sapiens |
| 1489 | AAH02403 | 3107 | 100 | BC002403 lectin, galactoside- | Homo sapiens |
| | | | | binding, soluble, 3 binding | |
| | | | | protein (galectin 6 binding | |
| | | | | protein) | |
| 1489 | AAH02998 | 3107 | 100 | BC002998 lectin, galactoside- | Homo sapiens |
| | | | | binding, soluble, 3 binding | |
| | | | | protein (galectin 6 binding | |
| | | | | protein) | |
| 1489 | AAH15761 | 3107 | 100 | BC015761 lectin, galactoside- | Homo sapiens |
| | | | | binding, soluble, 3 binding | |
| | | | | protein | |
| 1490 | AAH15032 | 2122 | 100 | BC015032 ribosomal protein L3 | Homo sapiens |
| 1490 | AAH15767 | 2122 | 100 | BC015767 ribosomal protein L3 | Homo sapiens |
| 1490 | BC008003 | 2122 | 100 | ribosomal protein L3 | Homo sapiens |
| 1491 | AF052694 | 2391 | 100 | alpha-tubulin | Meriones |
| | | | | | unguiculatus |
| 1491 | BC002219 | 2391 | 100 | Similar to tubulin alpha 1 | Mus musculus |
| 1491 | BC008117 | 2391 | 100 | tubulin alpha 1 | Mus musculus |
| 1492 | AAH21822 | 264 | 71 | BC021822 Unknown (protein for | Homo sapiens |
| | | | | MGC: 24986) | |
| 1492 | AK025116 | 265 | 71 | unnamed protein product | Homo sapiens |
| 1492 | AK090511 | 279 | 71 | unnamed protein product | Homo sapiens |
| 1493 | AAH02449 | 1196 | 99 | BC002449 Similar to CG10641 gene | Homo sapiens |
| | | | | product | |
| 1493 | AAH04128 | 1331 | 99 | BC004128 Unknown (protein for | Homo sapiens |
| | | | | IMAGE: 3940060) | |
| 1493 | AK023674 | 1199 | 100 | unnamed protein product | Homo sapiens |
| 1494 | AAH01537 | 2971 | 100 | BC001537 Unknown (protein for | Homo sapiens |
| | | | | MGC: 786) | |
| 1494 | AB021743 | 2968 | 99 | PR65 | Mus musculus |
| 1494 | 2C006606 | 2968 | 99 | protein phosphatase 2 (formerly | Mus musculus |
| | | | | 2A), regulatory subunit A (PR | |
| | | | | 65), alpha isoform | |
| 1495 | AAH09642 | 939 | 100 | BC009642 FXYD domain-containing | Homo sapiens |
| | | | | ion transport regulator 5 | |
| 1495 | AB072911 | 939 | 100 | dysadherin | Homo sapiens |
| 1495 | AF161462_1 | 923 | 98 | AF161462 HSPC113 | Homo sapiens |
| 1496 | AAH09687 | 869 | 100 | BC009687 epithelial membrane | Homo sapiens |
| | | | | protein 2 | |
| 1496 | AY057060 | 869 | 100 | epithelial membrane protein 2 | Homo sapiens |
| 1496 | U52100 | 869 | 100 | XMP | Homo sapiens |
| 1497 | AB012214 | 6727 | 78 | DNA cytosine 5 methyltransferase | Rattus |
| | | | | | Rattus |
| 1497 | AF180682 | 6869 | 100 | DNA (cytosine-5)- | Homo sapiens |
| | | | | methyltransferase | |
| 1497 | X63692 | 8587 | 100 | DNA (cytosine-5-)- | Homo sapiens |
| | | | | methyltransferase | |
| 1498 | AAH15961 | 961 | 100 | BC015961 adrenomedullin | Homo sapiens |
| 1498 | D14874 | 961 | 100 | adrenomedullin precursor | Homo sapiens |
| 1498 | S73906 | 961 | 100 | adrenomedullin; AM | Homo sapiens |
| 1499 | AAH15973 | 514 | 100 | BC015973 S100 calcium-binding | Homo sapiens |
| | | | | protein A10 (annexin II ligand, | |
| | | | | calpactin I, light polypeptide | |
| | | | | (p11)) | |
| 1499 | M38591 | 514 | 100 | cellular ligand of annexin II | Homo sapiens |
| 1499 | M81457 | 514 | 100 | calpactin I light chain | Homo sapiens |
| 1500 | AAH16056 | 1137 | 100 | BC016056 claudin 3 | Homo sapiens |
| 1500 | AB000714 | 1137 | 100 | RVP1 | Homo sapiens |
| 1500 | AF007189 | 1137 | 100 | claudin 3 | Homo sapiens |
| 1501 | AAH16142 | 1432 | 100 | BC016142 Similar to RIKEN cDNA | Homo sapiens |
| | | | | 2310032N20 gene | |
| 1501 | AAH20773 | 1432 | 100 | BC020773 Unknown (protein for | Homo sapiens |
| | | | | MGC: 22685) | |
| 1501 | AX405824 | 1432 | 100 | unnamed protein product | Homo sapiens |
| 1502 | AF315378_1 | 1955 | 98 | AF315378 suppressor of | Rattus |
| | | | | profilin/p41 of actin-related | norvegicus |
| | | | | complex 2/3 | |
| 1502 | BC039594 | 1964 | 99 | actin related protein 2/3 | Homo sapiens |
| | | | | complex, subunit 1A, 41 kDa | |
| 1502 | Y08999 | 1980 | 100 | Sop2p-like protein | Homo sapiens |
| 1503 | AF492677_1 | 383 | 100 | AF492677 odd homeobox 1 protein | Homo sapiens |
| | | | | isoform A | |
| 1503 | AF492680_1 | 383 | 100 | AF492680 odd homeobox 1 protein | Homo sapiens |
| | | | | isoform A | |
| 1503 | AF492681_1 | 383 | 100 | AF492681 odd homeobox 1 protein | Homo sapiens |
| | | | | isoform A | |
| 1504 | AK056992 | 2163 | 100 | unnamed protein product | Homo sapiens |
| 1504 | AX392121 | 2155 | 99 | unnamed protein product | Homo sapiens |
| 1504 | AY033237 | 2155 | 99 | alcohol dehydrogenase 8 | Homo sapiens |
| 1505 | AAH16748 | 485 | 100 | BC016748 ribosomal protein L37a | Homo sapiens |
| 1505 | X14069 | 485 | 100 | ribosomal protein L37a (AA 1-92 | Rattus |
| | | | | | Rattus |
| 1505 | X73331 | 485 | 100 | ribosomal protein L37a | Mus musculus |
| 1506 | AAH12807 | 3702 | 100 | BC012807 Unknown (protein for | Homo sapiens |
| | | | | MGC: 3483) | |
| 1506 | AAH14485 | 3702 | 100 | BC014485 Unknown (protein for | Homo sapiens |
| | | | | MGC: 23206) | |
| 1506 | AAH16753 | 3702 | 100 | BC016753 Unknown (protein for | Homo sapiens |
| | | | | MGC: 1138) | |
| 1507 | AAH15834 | 1724 | 100 | BC015834 annexin A2 | Homo sapiens |
| 1507 | AAH16774 | 1724 | 100 | BC016774 annexin A2 | Homo sapiens |
| 1507 | AAH21114 | 1724 | 100 | BC021114 annexin A2 | Homo sapiens |
| 1508 | AAH17343 | 644 | 100 | BC017343 ribosomal protein L31 | Homo sapiens |
| 1508 | BC008223 | 644 | 100 | ribosomal protein L31 | Mus musculus |
| 1508 | X04809 | 644 | 100 | ribosomal protein L31 (AA 1-125) | Rattus |
| | | | | | norvegicus |
| 1509 | AAH17378 | 2955 | 100 | BC017378 hexosaminidase B (beta | Homo sapiens |
| | | | | polypeptide) | |
| 1509 | AF378118_1 | 2955 | 100 | AF378118 cervical cancer proto- | Homo sapiens |
| | | | | oncogene 7 | |
| 1509 | M23294 | 2955 | 100 | beta-hexosaminidase beta-subunit | Homo sapiens |
| 1510 | AAH12168 | 1373 | 100 | BC012168 Similar to proteasome | Homo sapiens |
| | | | | (prosome, macropain) subunit, | |
| | | | | beta type, 4 | |
| 1510 | AAH17451 | 1373 | 100 | BC017451 proteasome (prosome, | Homo sapiens |
| | | | | macropain) subunit, beta type, 4 | |
| 1510 | AAH17486 | 1373 | 100 | BC017486 proteasome (prosome, | Homo sapiens |
| | | | | macropain) subunit, beta type, 4 | |
| 1511 | AAH06124 | 2618 | 100 | BC006124 IMP (inosine | Homo sapiens |
| | | | | monophosphate) dehydrogenase 2 | |
| 1511 | AAH12840 | 2618 | 100 | BC012840 IMP (inosine | Homo sapiens |
| | | | | monophosphate) dehydrogenase 2 | |
| 1511 | AAH15567 | 2618 | 100 | BC015567 IMP (inosine | Homo sapiens |
| | | | | monophosphate) dehydrogenase 2 | |
| 1512 | AAH07457 | 1103 | 100 | BC007457 hypothetical protein | Homo sapiens |
| 1512 | AAH20796 | 1103 | 100 | BC020796 hypothetical protein | Homo sapiens |
| | | | | HSPC177 | |
| 1512 | AAH21168 | 1103 | 100 | BC021168 hypothetical protein | Homo sapiens |
| | | | | HSPC177 | |
| 1513 | AAH04904 | 2894 | 99 | BC004904 nuclear RNA export | Homo sapiens |
| | | | | factor 1 | |
| 1513 | BC028041 | 2894 | 99 | nuclear RNA export factor 1 | Homo sapiens |
| 1513 | U80073 | 2905 | 100 | tip associating protein | Homo sapiens |
| 1514 | AF064238_1 | 4503 | 97 | AF064238 smoothelin large isoform | Homo sapiens |
| | | | | L2 | |
| 1514 | AY061971 | 4653 | 100 | smoothelin-B2 | Homo sapiens |
| 1514 | AY061972 | 4533 | 96 | smoothelin-B3 | Homo sapiens |
| 1515 | AAH17773 | 1214 | 100 | BC017773 triggering receptor | Homo sapiens |
| | | | | expressed on myeloid cells 1 | |
| 1515 | AF196329_1 | 1214 | 100 | AF196329 triggering receptor | Homo sapiens |
| | | | | expressed on monocytes 1 | |
| 1515 | AF287008_1 | 1214 | 100 | AF287008 triggering receptor | Homo sapiens |
| | | | | expressed on monocytes 1 | |
| 1516 | AAH17891 | 579 | 100 | BC017891 vesicle-associated | Homo sapiens |
| | | | | membrane protein 5 (myobrevin) | |
| 1516 | AF054825 | 579 | 100 | VAMP5 | Homo sapiens |
| 1516 | AF077197 | 579 | 100 | VAMP5-like protein | Homo sapiens |
| 1517 | AAH17931 | 819 | 100 | BC017931 Similar to RIKEN cDNA | Homo sapiens |
| | | | | 1110055A02 gene | |
| 1517 | AF400652_1 | 819 | 100 | AF400652 thyroid hormone receptor | Homo sapiens |
| | | | | interacting protein 3 | |
| 1517 | L40410 | 806 | 100 | thyroid receptor interactor | Homo sapiens |
| 1518 | AAH18732 | 1606 | 100 | BC018732 Unknown (protein for | Homo sapiens |
| | | | | MGC: 31847) | |
| 1518 | AF125533_1 | 1606 | 100 | AF125533 NADH-cytochrome b5 | Homo sapiens |
| | | | | reductase isoform | |
| 1518 | AX083421 | 1606 | 100 | unnamed protein product | Homo sapiens |
| 1519 | AAH02453 | 3263 | 100 | BC002453 heat shock 70 kD protein | Homo sapiens |
| | | | | 1A | |
| 1519 | AAH09322 | 3263 | 100 | BC009322 heat shock 70 kD protein | Homo sapiens |
| | | | | 1A | |
| 1519 | AAH18740 | 3263 | 100 | BC018740 heat shock 70 kD protein | Homo sapiens |
| | | | | 1A | |
| 1520 | 1680052_1 | 2856 | 100 | S62076 lysosomal enzyme beta-N- | Homo sapiens |
| | | | | acetylhexosaminidase A | |
| 1520 | AAH18927 | 2856 | 100 | BC018927 hexosaminidase A (alpha | Homo sapiens |
| | | | | polypeptide) | |
| 1520 | M16424 | 2856 | 100 | beta-hexosaminidase alpha chain | Homo sapiens |
| 1521 | AAH12992 | 2115 | 99 | BC012992 Similar to RNA binding | Homo sapiens |
| | | | | motif, single stranded | |
| | | | | interacting protein 1 | |
| 1521 | AAH12993 | 2115 | 99 | BC012993 Similar to RNA binding | Homo sapiens |
| | | | | motif, single stranded | |
| | | | | interacting protein 1 | |
| 1521 | AAH18951 | 2142 | 100 | BC018951 RNA binding motif, | Homo sapiens |
| | | | | single stranded interacting | |
| | | | | protein 1 | |
| 1522 | AAH16346 | 908 | 100 | BC016346 Unknown (protein for | Homo sapiens |
| | | | | MGC: 24392) | |
| 1522 | AAH16354 | 908 | 100 | BC016354 Unknown (protein for | Homo sapiens |
| | | | | MGC: 24480) | |
| 1522 | AAH18990 | 908 | 100 | BC018990 Unknown (protein for | Homo sapiens |
| | | | | MGC: 20176) | |
| 1523 | AAH01206 | 2235 | 99 | BC001206 Unknown (protein for | Homo sapiens |
| | | | | MGC: 3208) | |
| 1523 | AK025822 | 3505 | 99 | unnamed protein product | Homo sapiens |
| 1523 | AL833865 | 3414 | 100 | hypothetical protein | Homo sapiens |
| 1524 | AAH06772 | 769 | 100 | BC006772 ribosomal protein S13 | Homo sapiens |
| 1524 | BC029732 | 769 | 100 | ribosomal protein S13 | Homo sapiens |
| 1524 | X53378 | 769 | 100 | ribosomal protein S13 | Rattus |
| | | | | | Rattus |
| 1525 | J05192 | 1982 | 100 | alpha-actin | Homo sapiens |
| 1525 | X06801 | 1968 | 99 | alpha-actin (AA 1-377) | Rattus |
| | | | | | Rattus |
| 1525 | X13297 | 1968 | 99 | alpha-actin (AA 1-377) | Mus musculus |
| 1526 | AAH00260 | 1675 | 99 | BC000260 aldo-keto reductase | Homo sapiens |
| | | | | family 1, member B1 (aldose | |
| | | | | reductase) | |
| 1526 | AAH10391 | 1675 | 99 | BC010391 aldo-keto reductase | Homo sapiens |
| | | | | family 1, member B1 (aldose | |
| | | | | reductase) | |
| 1526 | J05017 | 1677 | 100 | aldose reductase | Homo sapiens |
| 1527 | M22246 | 2623 | 100 | aromatase precursor (EC | Homo sapiens |
| | | | | 1.14.14.1) | |
| 1527 | X13589 | 2623 | 100 | aromatase (AA 1-503) | Homo sapiens |
| 1527 | Y07508 | 2620 | 99 | aromatase (AA 1-503) | Homo sapiens |
| 1528 | M59916 | 3466 | 100 | acid sphingomyelinase | Homo sapiens |
| 1528 | M81780 | 3450 | 99 | acid sphingomyelinase | Homo sapiens |
| 1528 | X63600 | 3461 | 99 | acid sphingomyelinase | Homo sapiens |
| 1529 | AAH21993 | 1707 | 100 | BC021993 guanine nucleotide | Homo sapiens |
| | | | | binding protein (G protein), beta | |
| | | | | polypeptide 2-like 1 | |
| 1529 | M24193 | 1707 | 100 | MHC B complex protein 12.3 | Gallus |
| | | | | | gallus |
| 1529 | X75313 | 1707 | 100 | B complex protein mRNA 12-3 | Mus musculus |
| 1530 | AAH05358 | 642 | 100 | BC005358 non-histone chromosome | Homo sapiens |
| | | | | protein 2 (S. cerevisiae)-like 1 | |
| 1530 | AAH19282 | 642 | 100 | BC019282 non-histone chromosome | Homo sapiens |
| | | | | protein 2 (S. cerevisiae)-like 1 | |
| 1530 | BC026755 | 642 | 100 | Similar to sperm specific antigen 1 | Mus musculus |
| 1531 | AAH19296 | 2747 | 100 | BC019296 chaperonin containing | Homo sapiens |
| | | | | TCP1, subunit 7 (eta) | |
| 1531 | AF026292 | 2747 | 100 | chaperonin containing t-complex | Homo sapiens |
| | | | | polypeptide 1, eta subunit; CCT- | |
| | | | | eta | |
| 1531 | BC008255 | 2628 | 95 | chaperonin subunit 7 (eta) | Mus musculus |
| 1532 | BC035220 | 3879 | 99 | similar to complement component | Homo sapiens |
| 1532 | M14058 | 3885 | 100 | human complement C1r | Homo sapiens |
| 1532 | X04701 | 3879 | 99 | precursor of C1r (AA −17 to 688) | Homo sapiens |
| 1533 | J04080 | 3789 | 100 | complement component C1s | Homo sapiens |
| 1533 | M18767 | 3789 | 100 | complement subcomponent C1s | Homo sapiens |
| | | | | precursor | |
| 1533 | X06596 | 3789 | 100 | complement protein C1s precursor | Homo sapiens |
| 1534 | AAH03175 | 2072 | 100 | BC003175 N-myc downstream | Homo sapiens |
| | | | | regulated | |
| 1534 | AF004162 | 2072 | 100 | nickel-specific induction protein | Homo sapiens |
| 1534 | D87953 | 2072 | 100 | RTP | Homo sapiens |
| 1535 | AF085250 | 759 | 100 | calmodulin | Perca |
| | | | | | flavescens |
| 1535 | D83350 | 759 | 100 | calmodulin | Anas |
| | | | | | platyrhynchos |
| 1535 | K01945 | 759 | 100 | calmodulin (cDNA clone 71) | Xenopus |
| | | | | | laevis |
| 1536 | AAH07911 | 2282 | 100 | BC007911 calreticulin | Homo sapiens |
| 1536 | AAH20493 | 2282 | 100 | BC020493 calreticulin | Homo sapiens |
| 1536 | AY047586 | 2282 | 100 | calreticulin | Homo sapiens |
| 1537 | AAH16300 | 533 | 100 | BC016300 S100 calcium-binding | Homo sapiens |
| | | | | protein A4 (calcium protein, | |
| | | | | calvasculin, metastasin, murine | |
| | | | | placental homolog) | |
| 1537 | Z18950 | 533 | 100 | CAPL | Homo sapiens |
| 1537 | Z33457 | 533 | 100 | mts1 | Homo sapiens |
| 1538 | AAH01506 | 710 | 100 | BC001506 CD59 antigen p18-20 | Homo sapiens |
| | | | | (antigen identified by monoclonal | |
| | | | | antibodies 16.3A5, EJ16, EJ30, | |
| | | | | EL32 and G344) | |
| 1538 | X16447 | 710 | 100 | precursor polypeptide (AA −25 to | Homo sapiens |
| | | | | 103) | |
| 1538 | X17198 | 710 | 100 | CD59 antigen precursor | Homo sapiens |
| 1539 | AAH06290 | 898 | 100 | BC006290 chorionic gonadotropin, | Homo sapiens |
| | | | | beta polypeptide | |
| 1539 | BC022796 | 898 | 100 | Unknown (protein for MGC: 39080) | Homo sapiens |
| 1539 | BC030994 | 898 | 100 | chorionic gonadotropin, beta | Homo sapiens |
| | | | | polypeptide | |
| 1540 | J03225 | 1674 | 100 | lipoprotein-associated | Homo sapiens |
| | | | | coagulation inhibitor precursor | |
| 1540 | M58650 | 1674 | 100 | lipoprotein associated | Homo sapiens |
| | | | | coagulation inhibitor | |
| 1540 | M59499 | 1674 | 100 | lipoprotein-associated | Homo sapiens |
| | | | | coagulation inhibitor | |
| 1540 | M59499 | 1674 | 100 | lipoprotein-associated | Homo sapiens |
| | | | | coagulation inhibitor | |
| 1541 | AAH14167 | 1072 | 100 | BC014167 Unknown (protein for | Homo sapiens |
| | | | | MGC: 20892) | |
| 1541 | AAH20804 | 1072 | 100 | BC020804 ribosomal protein L13 | Homo sapiens |
| 1541 | BC027463 | 1072 | 100 | ribosomal protein L13 | Homo sapiens |
| 1542 | AAH21136 | 1333 | 100 | BC021136 Unknown (protein for | Homo sapiens |
| | | | | MGC: 32056) | |
| 1542 | AF097362_1 | 1333 | 100 | AF097362 gamma-interferon | Homo sapiens |
| | | | | inducible lysosomal thiol | |
| | | | | reductase | |
| 1542 | BC031020 | 1329 | 99 | interferon, gamma-inducible | Homo sapiens |
| | | | | protein 30 | |
| 1543 | AAH07008 | 929 | 100 | BC007008 crystallin, alpha B | Homo sapiens |
| 1543 | M28638 | 929 | 100 | alpha-B2-crystallin | Homo sapiens |
| 1543 | S45630 | 929 | 100 | alpha B-crystallin | Homo sapiens |
| 1544 | BC003775 | 1169 | 99 | casein kinase II, beta subunit | Mus musculus |
| 1544 | M30448 | 1174 | 100 | casein kinase II beta subunit | Homo sapiens |
| 1544 | M59458 | 1169 | 99 | casein kinase II beta subunit | Gallus |
| | | | | | gallus |
| 1545 | M77820 | 6209 | 50 | fibronectin | Xenopus |
| | | | | | laevis |
| 1545 | X02761 | 12570 | 99 | fibronectin precursor | Homo sapiens |
| 1545 | X15906 | 7305 | 65 | precursor polypeptide (AA −32 to | Rattus |
| | | | | 2445) | norvegicus |
| 1546 | BC032329 | 2740 | 99 | cytochrome P450, subfamily XIA | Homo sapiens |
| | | | | (cholesterol side chain cleavage) | |
| 1546 | M14565 | 2753 | 100 | cholesterol side-chain cleavage | Homo sapiens |
| | | | | enzyme P450scc (EC 1.14.15.67) | |
| 1546 | X05367 | 2734 | 99 | desmolase | Homo sapiens |
| 1547 | AF318372_1 | 592 | 99 | AF318372 unknown | Homo sapiens |
| 1547 | AJ318881 | 601 | 100 | XAGE-3 protein | Homo sapiens |
| 1547 | AJ318893 | 601 | 100 | XAGE-3 protein | Homo sapiens |
| 1548 | AAH18641 | 2406 | 100 | BC018641 eukaryotic translation | Homo sapiens |
| | | | | elongation factor 1 alpha 1 | |
| 1548 | AAH21686 | 2406 | 100 | BC021686 eukaryotic translation | Homo sapiens |
| | | | | elongation factor 1 alpha 1 | |
| 1548 | BC028674 | 2406 | 100 | eukaryotic translation elongation | Homo sapiens |
| | | | | factor 1 alpha 1 | |
| 1549 | AAH07044 | 2377 | 100 | BC007044 fibrinogen, gamma | Homo sapiens |
| | | | | polypeptide | |
| 1549 | AAH21674 | 2377 | 100 | BC021674 fibrinogen, gamma | Homo sapiens |
| | | | | polypeptide | |
| 1549 | AF350254_1 | 2377 | 100 | AF350254 fibrinogen gamma chain, | Homo sapiens |
| | | | | isoform gamma-A precursor | |
| 1550 | AAH07785 | 2357 | 100 | BC007785 Unknown (protein for | Homo sapiens |
| | | | | IMAGE: 2819608) | |
| 1550 | AAH21565 | 2387 | 100 | BC021565 Unknown (protein for | Homo sapiens |
| | | | | MGC: 31904) | |
| 1550 | AF121858_1 | 2387 | 100 | AF121858 sorting nexin 8 | Homo sapiens |
| 1551 | AAH05929 | 1222 | 100 | BC005929 proteoglycan 2, bone | Homo sapiens |
| | | | | narrow (natural killer cell | |
| | | | | activator, eosinophil granule | |
| | | | | major basic protein) | |
| 1551 | X14088 | 1222 | 100 | major basic protein precusor (AA | Homo sapiens |
| | | | | 1-222) | |
| 1551 | Y00809 | 1222 | 100 | major basic preproprotein (AA −15 | Homo sapiens |
| | | | | to 207) | |
| 1552 | AL050169 | 951 | 100 | hypothetical protein | Homo sapiens |
| 1552 | M34046 | 951 | 100 | placental protein 14 precursor | Homo sapiens |
| | | | | (PP14) | |
| 1552 | M61886 | 951 | 100 | pregnancy-associated endometrial | Homo sapiens |
| | | | | alpha2-globulin | |
| 1553 | AAH15641 | 2224 | 100 | BC015641 enolase 1, (alpha) | Homo sapiens |
| 1553 | BC022545 | 2224 | 100 | enolase 1, (alpha) | Homo sapiens |
| 1553 | BC027725 | 2224 | 100 | enolase 1, (alpha) | Homo sapiens |
| 1554 | AF272142 | 2649 | 100 | cytochrome P450 | Homo sapiens |
| 1554 | BC032594 | 2649 | 100 | cytochrome P450, subfamily IIJ | Homo sapiens |
| | | | | (arachidonic acid epoxygenase) | |
| | | | | polypeptide 2 | |
| 1554 | U37143 | 2649 | 100 | cytochrome P450 monooxygenase | Homo sapiens |
| | | | | CYP2J2 | |
| 1555 | AAH17123 | 1367 | 100 | BC017123 ribosomal protein S3A | Homo sapiens |
| 1555 | AAH19072 | 1367 | 100 | BC019072 ribosomal protein S3A | Homo sapiens |
| 1555 | BC030161 | 1367 | 100 | ribosomal protein S3A | Homo sapiens |
| 1556 | AB042200 | 2004 | 88 | low-density lipoprotein receptor- | Mus musculus |
| | | | | related protein 9 | |
| 1556 | AL834518 | 2154 | 99 | hypothetical protein | Homo sapiens |
| 1556 | AX359701 | 2268 | 99 | unnamed protein product | Homo sapiens |
| 1557 | BC040431 | 5104 | 99 | glucosidase, alpha; acid (Pompe | Homo sapiens |
| | | | | disease, glycogen storage disease | |
| | | | | type II) | |
| 1557 | M34424 | 5105 | 100 | acid alpha-glucosidase | Homo sapiens |
| 1557 | X55080 | 5105 | 100 | acid alpha-glucosidase | Homo sapiens |
| 1558 | AAH10914 | 768 | 100 | BC010914 Similar to hemoglobin, | Homo sapiens |
| | | | | gamma G | |
| 1558 | BC029387 | 768 | 100 | hemoglobin, gamma G | Homo sapiens |
| 1558 | M91037 | 768 | 100 | G-gamma globin | Homo sapiens |
| 1559 | AY148100 | 4568 | 100 | colony stimulating factor 3 | Homo sapiens |
| | | | | receptor (granulocyte) | |
| 1559 | M59818 | 4568 | 100 | granulocyte colony-stimulating | Homo sapiens |
| | | | | factor receptor | |
| 1559 | X55721 | 4568 | 100 | granulocyte colony stimulating | Homo sapiens |
| | | | | factor receptor 25-1 | |
| 1560 | AAH00013 | 1579 | 100 | BC000013 insulin-like growth | Homo sapiens |
| | | | | factor binding protein 3 | |
| 1560 | AAH18962 | 1579 | 100 | BC018962 insulin-like growth | Homo sapiens |
| | | | | factor binding protein 3 | |
| 1560 | M35878 | 1579 | 100 | growth factor-binding protein-3 | Homo sapiens |
| | | | | precursor | |
| 1561 | M23595 | 1413 | 100 | insulin-like growth factor | Homo sapiens |
| | | | | binding protein 1 | |
| 1561 | M59316 | 1413 | 100 | insulin-like growth factor | Homo sapiens |
| | | | | binding protein-1 | |
| 1561 | X12385 | 1413 | 100 | PP12 precorsor (AA −25 to 234) | Homo sapiens |
| 1562 | BC041218 | 676 | 100 | H3 histone, family 3B | Xenopus |
| | | | | | laevis |
| 1562 | M11393 | 676 | 100 | histone 3.3 | Gallus |
| | | | | | gallus |
| 1562 | Y00392 | 676 | 100 | histone H3.3B (AA 1-136) | Gallus |
| | | | | | gallus |
| 1563 | AF523281 | 1916 | 99 | MHC class Ib antigen | Homo sapiens |
| 1563 | AF523283 | 1916 | 99 | MHC class Ib antigen | Homo sapiens |
| 1563 | M20022 | 1922 | 100 | HLA-E class I protein precursor | Homo sapiens |
| 1564 | AAH03569 | 1980 | 100 | BC003569 E74-like factor 3 (ets | Homo sapiens |
| | | | | domain transcription factor, | |
| | | | | epithelial-specific) | |
| 1564 | AF017307 | 1980 | 100 | Ets-related transcription factor | Homo sapiens |
| 1564 | AF110184_1 | 1980 | 100 | AF110184 epithelium-restricted | Homo sapiens |
| | | | | Ets protein ESX | |
| 1565 | AF523305 | 1810 | 100 | MHC class Ib antigen | Homo sapiens |
| 1565 | AF523306 | 1810 | 100 | MHC class Ib antigen | Homo sapiens |
| 1565 | AF523309 | 1810 | 100 | MHC class Ib antigen | Homo sapiens |
| 1566 | AAH09507 | 836 | 99 | BC009507 Unknown (protein for | Homo sapiens |
| | | | | MGC: 3945) | |
| 1566 | AY168648 | 836 | 99 | ubiquitin-like protein ISG15 | Homo sapiens |
| 1566 | M13755 | 839 | 100 | 17-kDa protein | Homo sapiens |
| 1567 | AB033054 | 2567 | 94 | KIAA1228 protein | Homo sapiens |
| 1567 | AF099138 | 1352 | 50 | GLUT4 vesicle protein | Rattus |
| | | | | | norvegicus |
| 1567 | BC011482 | 1348 | 50 | Similar to membrane bound C2 | Mus musculus |
| | | | | domain containing protein | |
| 1568 | AAH10853 | 1129 | 100 | BC010853 Similar to integral type | Homo sapiens |
| | | | | I protein | |
| 1568 | AAH17495 | 1129 | 100 | BC017495 integral type I protein | Homo sapiens |
| 1568 | BC022232 | 1129 | 100 | integral type I protein | Homo sapiens |
| 1569 | AF357203_1 | 1832 | 97 | AF357203 MAP kinase phosphatase-1 | Rattus |
| | | | | | norvegicus |
| 1569 | BC022463 | 1886 | 100 | dual specificity phosphatase 1 | Homo sapiens |
| 1569 | X68277 | 1886 | 100 | protein-tyrosine phosphatase | Homo sapiens |
| 1570 | AAH09407 | 687 | 100 | BC009407 ribosomal protein S17 | Homo sapiens |
| 1570 | AAH19899 | 687 | 100 | BC019899 ribosomal protein S17 | Homo sapiens |
| 1570 | BC022370 | 687 | 100 | ribosomal protein S17 | Homo sapiens |
| 1571 | AAH15105 | 1370 | 100 | BC015105 proteasome (prosome, | Homo sapiens |
| | | | | macropain) subunit, alpha type, 1 | |
| 1571 | AAH15356 | 1370 | 100 | BC015356 proteasome (prosome, | Homo sapiens |
| | | | | macropain) subunit, alpha type, 1 | |
| 1571 | BC022372 | 1370 | 100 | proteasome (prosome, macropain) | Homo sapiens |
| | | | | subunit, alpha type, 1 | |
| 1572 | AAH03352 | 723 | 100 | BC003352 tumor protein, | Homo sapiens |
| | | | | translationally-controlled 1 | |
| 1572 | BC022436 | 723 | 100 | Similar to tumor protein, | Homo sapiens |
| | | | | translationally-controlled 1 | |
| 1572 | X16064 | 723 | 100 | tumor protein (AA 1-172) | Homo sapiens |
| 1573 | BC023006 | 3271 | 100 | Unknown (protein for MGC: 30059) | Homo sapiens |
| 1573 | M27024 | 3271 | 100 | heat shock protein | Homo sapiens |
| 1573 | X15183 | 3271 | 100 | 90 kDa heat-shock protein (AA 1-732) | Homo sapiens |
| 1574 | AAH08064 | 2806 | 100 | BC008064 monoamine oxidase A | Homo sapiens |
| 1574 | M68857 | 2806 | 100 | monoamine oxidase A | Homo sapiens |
| 1574 | M69226 | 2806 | 100 | monoamine oxidase A | Homo sapiens |
| 1575 | M84378 | 1952 | 100 | lymphocyte antigen | Homo sapiens |
| 1575 | M84379 | 1945 | 99 | MHC class I lymphocyte antigen | Homo sapiens |
| 1575 | Z27120 | 1946 | 99 | HLA-A2 SLU | Homo sapiens |
| 1576 | BC032350 | 1338 | 100 | major histocompatibility complex, | Homo sapiens |
| | | | | class II, DR alpha | |
| 1576 | M60334 | 1338 | 100 | cell surface glycoprotein | Homo sapiens |
| 1576 | Z84814 | 1338 | 100 | dJ172K2.4.1 (major | Homo sapiens |
| | | | | histocompatibility complex, class | |
| | | | | II, DR alpha, isoform 1) | |
| 1577 | AAH02476 | 803 | 100 | BC002476 non-metastatic cells 2, | Homo sapiens |
| | | | | protein (NM23B) expressed in | |
| 1577 | L16785 | 803 | 100 | c-myc transcription factor | Homo sapiens |
| 1577 | X58965 | 803 | 100 | NM23-H2 protein | Homo sapiens |
| 1578 | AAH11645 | 1585 | 100 | BC011645 Similar to Ras-related | Homo sapiens |
| | | | | associated with diabetes | |
| 1578 | L24564 | 1585 | 100 | Rad | Homo sapiens |
| 1578 | U46165 | 1565 | 99 | Rad GTPase | Homo sapiens |
| 1579 | AAH03358 | 1151 | 100 | BC003358 ribosonal protein L10 | Homo sapiens |
| 1579 | BC026276 | 1151 | 100 | ribosomal protein L10 | Homo sapiens |
| 1579 | L44140 | 1151 | 100 | QM | Homo sapiens |
| 1580 | A84527 | 2912 | 100 | unnamed protein product | unidentified |
| 1580 | AX024042 | 2912 | 100 | fusion between Aeguorea victoria | synthetic |
| | | | | and human | construct |
| 1580 | M62399 | 2912 | 100 | NF-kappa-B transcription factor | Homo sapiens |
| 1581 | AAH10860 | 2071 | 100 | BC010860 serine (or cysteine) | Homo sapiens |
| | | | | proteinase inhibitor, clade E | |
| | | | | (nexin, plasminogen activator | |
| | | | | inhibitor type 1), member 1 | |
| 1581 | AF386492_1 | 2071 | 100 | AF386492 serine-cysteine | Homo sapiens |
| | | | | proteinase inhibitor clade E | |
| | | | | member 1 | |
| 1581 | X04429 | 2071 | 100 | precursor polypeptide | Homo sapiens |
| 1582 | AAH00491 | 1316 | 100 | BC000491 proliferating cell | Homo sapiens |
| | | | | nuclear antigen | |
| 1582 | AF347680_1 | 1316 | 100 | AF347680 proliferating cell | Macaca |
| | | | | nuclear antigen | fascicularis |
| 1582 | AL121924 | 1316 | 100 | dJ746J20.2 (proliferating cell | Homo sapiens |
| | | | | nuclear antigen) | |
| 1583 | AC007165 | 2130 | 100 | interleukin-1 receptor type II | Homo sapiens |
| 1583 | AY124010 | 2130 | 100 | interleukin 1 receptor, type II | Homo sapiens |
| 1583 | X59770 | 2130 | 100 | type II interleukin-1 receptor | Homo sapiens |
| 1584 | BC041071 | 2366 | 100 | protein phosphatase 2 (formerly | Homo sapiens |
| | | | | 2A), regulatory subunit B (PR | |
| | | | | 52), alpha isoform | |
| 1584 | M64929 | 2366 | 100 | protein phosphatase-2A subunit- | Homo sapiens |
| | | | | alpha | |
| 1584 | U09356 | 2366 | 100 | protein phosphatase 2A1 B alpha | Oryctolagus |
| | | | | subunit | cuniculus |
| 1585 | M34421 | 2279 | 99 | pregnancy-specific beta-1 | Homo sapiens |
| | | | | glycoprotein precursor | |
| 1585 | M34481 | 2279 | 99 | pregnancy-specific beta-1- | Homo sapiens |
| | | | | glycoprotein 11 | |
| 1585 | M58591 | 2291 | 100 | PSG11 | Homo sapiens |
| 1586 | BC018254 | 703 | 97 | retinol binding protein 1, | Mus musculus |
| | | | | cellular | |
| 1586 | M11433 | 724 | 100 | retinol-binding protein | Homo sapiens |
| 1586 | X07437 | 724 | 100 | cellular retinol binding protein | Homo sapiens |
| 1587 | J03149 | 4237 | 83 | M-CSF receptor | Felis catus |
| 1587 | U63963 | 5158 | 100 | CSF-1 receptor | Homo sapiens |
| 1587 | X03663 | 5150 | 99 | put. c-fms precursor | Homo sapiens |
| 1588 | BC005580 | 902 | 100 | polymerase (RNA) II (DNA | Mus musculus |
| | | | | directed) polypeptide G | |
| 1588 | U52427 | 902 | 100 | RNA polymerase II seventh subunit | Homo sapiens |
| 1588 | Z71925 | 902 | 100 | RNA polymerase II RPB7 subunit- | Rattus |
| | | | | like protein | norvegicus |
| 1589 | AAH11601 | 658 | 100 | BC011601 interferon, alpha- | Homo sapiens |
| | | | | inducible protein (clone IFI-6- | |
| | | | | 16) | |
| 1589 | AAH15603 | 658 | 100 | BC015603 interferon, alpha- | Homo sapiens |
| | | | | inducible protein (clone IFI-6- | |
| | | | | 16) | |
| 1589 | D11384 | 652 | 98 | ORF | Pan |
| | | | | | troglodytes |
| 1590 | AF147083 | 1683 | 100 | gamma-glutamyl hydrolase | Homo sapiens |
| 1590 | BC025025 | 1683 | 100 | gamma-glutamyl hydrolase | Homo sapiens |
| | | | | (conjugase, | |
| | | | | folylpolygammaglutamyl hydrolase) | |
| 1590 | U55206 | 1683 | 100 | human gamma-glutamyl hydrolase | Homo sapiens |
| 1591 | AAH10418 | 1552 | 100 | BC010418 laminin receptor 1 | Homo sapiens |
| | | | | (67 kD, ribosomal protein SA) | |
| 1591 | AAH13827 | 1552 | 100 | BC013827 laminin receptor 1 | Homo sapiens |
| | | | | (67 kD, ribosomal protein SA) | |
| 1591 | U43901 | 1552 | 100 | 37 kD laminin receptor | Homo sapiens |
| | | | | precursor/p40 ribosome associated | |
| | | | | protein | |
| 1592 | BC026033 | 4101 | 100 | gelsolin (amyloidosis, Finnish | Homo sapiens |
| | | | | type) | |
| 1592 | X04412 | 4101 | 100 | plasma gelsolin | Homo sapiens |
| 1592 | X13871 | 3849 | 94 | gelsolin | Sus scrofa |
| 1593 | AF304463_1 | 2813 | 100 | AF304463 calcium transport | Homo sapiens |
| | | | | protein CaT1 | |
| 1593 | AF365927_1 | 2813 | 100 | AF365927 calcium transport | Homo sapiens |
| | | | | protein CaT1 | |
| 1593 | AF365928_1 | 2946 | 100 | AF365928 calcium transport | Homo sapiens |
| | | | | protein CaT1 | |
| 1594 | AY040542 | 2353 | 100 | sialic acid binding | Homo sapiens |
| | | | | immunoglobulin-like lectin 6 | |
| 1594 | D86358 | 2353 | 100 | CD33L1 | Homo sapiens |
| 1594 | U71382 | 2246 | 97 | OB binding protein-1 | Homo sapiens |
| 1595 | AF286534_1 | 1115 | 100 | AF286534 GTP-binding protein | Rattus |
| | | | | RAB11B | norvegicus |
| 1595 | AF498947_1 | 1115 | 100 | AF498947 small GTP binding | Homo sapiens |
| | | | | protein RAB11B | |
| 1595 | X79780 | 1110 | 99 | YPT3 | Homo sapiens |
| 1596 | BC010604 | 1253 | 99 | ribosomal protein S6 | Mus musculus |
| 1596 | BC027620 | 1256 | 100 | ribosomal protein S6 | Homo sapiens |
| 1596 | Z54209 | 1253 | 99 | rpS6 | Mus musculus |
| 1597 | AJ005654 | 703 | 100 | secondary lymphoid tissue | Homo sapiens |
| | | | | chemokine | |
| 1597 | BC027918 | 703 | 100 | small inducible cytokine | Homo sapiens |
| | | | | subfamily A (Cys—Cys), member 21 | |
| 1597 | U88320 | 703 | 100 | beta chemokine Exodus-2 | Homo sapiens |
| 1598 | AF148856_2 | 6613 | 98 | AF148856 unknown | Homo sapiens |
| 1598 | AF149422_2 | 6615 | 98 | AF149422 unknown | Homo sapiens |
| 1598 | U93564 | 6714 | 100 | putative p150 | Homo sapiens |
| 1599 | AK012013 | 1608 | 100 | unnamed protein product | Mus musculus |
| 1599 | BC003742 | 1608 | 100 | 26S proteasome-associated pad1 | Mus musculus |
| | | | | homolog | |
| 1599 | U86782 | 1608 | 100 | 26S proteasome-associated pad1 | Homo sapiens |
| | | | | homolog | |
| 1600 | BC041218 | 676 | 100 | H3 histone, family 3B | Xenopus |
| | | | | | laevis |
| 1600 | M11393 | 676 | 100 | histone 3.3 | Gallus |
| | | | | | gallus |
| 1600 | Y00392 | 676 | 100 | histone H3.3B (AA 1-136) | Gallus |
| | | | | | gallus |
| 1601 | AAH11262 | 2716 | 99 | BC011262 Unknown (protein for | Homo sapiens |
| | | | | MGC: 18226) | |
| 1601 | AF006043 | 2719 | 100 | 3-phosphoglycerate dehydrogenase | Homo sapiens |
| 1601 | AF171237_1 | 2719 | 100 | AF171237 3-phosphoglycerate | Homo sapiens |
| | | | | dehydrogenase | |
| 1602 | AF109197_1 | 1254 | 100 | AF109197 nuclear chloride channel | Homo sapiens |
| 1602 | AJ012008 | 1251 | 99 | RNCC protein | Homo sapiens |
| 1602 | U93205 | 1254 | 100 | nuclear chloride ion channel | Homo sapiens |
| | | | | protein | |
| 1603 | BC029402 | 1311 | 100 | proteasome (prosome, macropain) | Homo sapiens |
| | | | | subunit, alpha type, 3 | |
| 1603 | BC038990 | 1307 | 99 | Similar to proteasome (prosome, | Homo sapiens |
| | | | | macropain) subunit, alpha type, 3 | |
| 1603 | D00762 | 1307 | 99 | proteasome subunit C8 | Homo sapiens |
| 1604 | AB062400 | 671 | 100 | Similar to ribosomal protein S15a | Homo sapiens |
| 1604 | BC030569 | 671 | 100 | ribosomal protein S15a | Homo sapiens |
| 1604 | X77953 | 671 | 100 | ribosomal protein S15a | Rattus |
| | | | | | norvegicus |
| 1605 | AB062438 | 1926 | 100 | OK/SW-CL.30 | Homo sapiens |
| 1605 | BC025832 | 1581 | 90 | Unknown (protein for MGC: 37936) | Mus musculus |
| 1605 | BC036872 | 1912 | 99 | Unknown (protein for | Homo sapiens |
| | | | | IMAGE: 5225889) | |
| 1606 | AAH16706 | 2319 | 100 | BC016706 eukaryotic translation | Homo sapiens |
| | | | | initiation factor 3, subunit 6 | |
| | | | | (48 kD) | |
| 1606 | AAH21679 | 2319 | 100 | BC021679 eukaryotic translation | Homo sapiens |
| | | | | initiation factor 3, subunit 6 | |
| | | | | (48 kD) | |
| 1606 | BC029177 | 2319 | 100 | eukaryotic translation initiation | Mus musculus |
| | | | | factor 3, subunit 6 (48 kD) | |
| 1607 | AAH04138 | 1465 | 99 | BC004138 ribosomal protein L6 | Homo sapiens |
| 1607 | BC031009 | 1475 | 100 | ribosomal protein L6 | Homo sapiens |
| 1607 | BC032299 | 1465 | 99 | ribosomal protein L6 | Homo sapiens |
| 1608 | AAH15813 | 2331 | 100 | BC015813 eukaryotic translation | Homo sapiens |
| | | | | elongation factor 1 gamma | |
| 1608 | BC028179 | 2331 | 100 | eukaryotic translation elongation | Homo sapiens |
| | | | | factor 1 gamma | |
| 1608 | BC031012 | 2331 | 100 | eukaryotic translation elongation | Homo sapiens |
| | | | | factor 1 gamma | |
| 1609 | AAH04927 | 1051 | 100 | BC004927 Unknown (protein for | Homo sapiens |
| | | | | MGC: 10432) | |
| 1609 | AF013711 | 1051 | 100 | 22 kDa actin-binding protein | Homo sapiens |
| 1609 | D84342 | 1051 | 100 | SM22 alpha | Homo sapiens |
| 1610 | AB007510 | 12543 | 100 | PRP8 protein | Homo sapiens |
| 1610 | AB047391 | 12461 | 99 | pre-mRNA processing 8 protein | Mus musculus |
| 1610 | AF092565 | 12477 | 99 | splicing factor Prp8 | Homo sapiens |
| 1611 | AAH10034 | 3127 | 100 | BC010034 thyroid autoantigen 70 kD | Homo sapiens |
| | | | | (Ku antigen) | |
| 1611 | AAH12154 | 3127 | 100 | BC012154 Similar to thyroid | Homo sapiens |
| | | | | autoantigen 70 kD (Ku antigen) | |
| 1611 | AAH18259 | 3127 | 100 | BC018259 thyroid autoantigen 70 kD | Homo sapiens |
| | | | | (Ku antigen) | |
| 1612 | AL050369 | 2492 | 99 | hypothetical protein | Homo sapiens |
| 1612 | AY040822 | 2500 | 100 | U4/U6 snRNP-associated 61 KDa | Homo sapiens |
| | | | | protein | |
| 1612 | BC018376 | 2479 | 98 | Unknown (protein for MGC: 19247) | Mus musculus |
| 1613 | AF343440_1 | 454 | 98 | AF343440 lysosomal H+ | Canis |
| | | | | transporting-ATPase subunit M9.2 | familiaris |
| 1613 | AX400012 | 459 | 100 | unnamed protein product | Homo sapiens |
| 1613 | Y15286 | 459 | 100 | vacuolar proton-ATPase subunit | Homo sapiens |
| | | | | M9.2 | |
| 1614 | AAH01273 | 1831 | 99 | BC001273 galactose-4-epimerase, | Homo sapiens |
| | | | | UDP- | |
| 1614 | AF022382 | 1835 | 100 | UDP-galactose 4′ epimerase | Homo sapiens |
| 1614 | L41668 | 1835 | 100 | UDP-galactose-4-epimerase | Homo sapiens |
| 1615 | AAH05270 | 900 | 99 | BC005270 NADH dehydrogenase | Homo sapiens |
| | | | | (ubiquinone) Fe-S protein 4 | |
| | | | | (18 kD) (NADH-coenzyme Q | |
| | | | | reductase) | |
| 1615 | AF020351 | 904 | 100 | NADH: ubiquinone oxidoreductase 18 kDa | Homo sapiens |
| | | | | IP subunit | |
| 1615 | X63215 | 839 | 90 | NADH dehydrogenase | Bos taurus |
| 1616 | AAH04963 | 674 | 100 | BC004963 ATP synthase, H+ | Homo sapiens |
| | | | | transporting, mitochondrial F0 | |
| | | | | complex, subunit c (subunit 9), | |
| | | | | isoform 1 | |
| 1616 | AL080089 | 674 | 100 | hypothetical protein | Homo sapiens |
| 1616 | X69907 | 674 | 100 | P1 gene for c subunit of human | Homo sapiens |
| | | | | mitochondrial ATP synthase | |
| 1617 | AF100620_1 | 1492 | 100 | AF100620 transcription factor- | Homo sapiens |
| | | | | like protein MRGX | |
| 1617 | AF167174_1 | 1492 | 100 | AF167174 MSL3-2 protein | Homo sapiens |
| 1617 | AL049610 | 1492 | 100 | dJ1055C14.2 (KIAA0026 | Homo sapiens |
| | | | | (transcription factor-like | |
| | | | | protein MRGX)) | |
| 1618 | AAH15557 | 1854 | 99 | BC015557 Unknown (protein for | Homo sapiens |
| | | | | MGC: 1567) | |
| 1618 | BC021637 | 1140 | 66 | CD68 antigen | Mus musculus |
| 1618 | S57235 | 1858 | 100 | CD68 | Homo sapiens |
| 1619 | L27631 | 4010 | 100 | amyloid precursor-like protein 2 | Homo sapiens |
| 1619 | S60099 | 4010 | 100 | APPH | Homo sapiens |
| 1619 | Z22572 | 4010 | 100 | CDEI binding protein | Homo sapiens |
| 1620 | AAH08850 | 1273 | 99 | BC008850 Unknown (protein for | Homo sapiens |
| | | | | MGC: 14308) | |
| 1620 | AAH09599 | 1273 | 99 | BC009599 Unknown (protein for | Homo sapiens |
| | | | | MGC: 14690) | |
| 1620 | L16558 | 1276 | 100 | ribosomal protein L7 | Homo sapiens |
| 1621 | AF062482 | 845 | 99 | SDP3 | Mus musculus |
| 1621 | AF062483 | 852 | 100 | SDP3 | Homo sapiens |
| 1621 | AF520420 | 838 | 98 | sorting nexin 3 | Mus musculus |
| 1622 | AF063657 | 6995 | 100 | vascular endothelial growth | Homo sapiens |
| | | | | factor receptor | |
| 1622 | D28498 | 5882 | 82 | Fit-1 tyrosine kinase receptor | Rattus |
| | | | | | norvegicus |
| 1622 | X51602 | 6989 | 99 | flt gene product (AA 1-1338) | Homo sapiens |
| 1623 | AF149422_2 | 703 | 49 | AF149422 unknown | Homo sapiens |
| 1623 | U93572 | 710 | 50 | putative p150 | Homo sapiens |
| 1623 | U93574 | 709 | 49 | putative p150 | Homo sapiens |
| 1624 | AF029729 | 3066 | 100 | neuralized | Homo sapiens |
| 1624 | BC026336 | 3041 | 99 | neuralized-like (Drosophila) | Homo sapiens |
| 1624 | U87864 | 3066 | 100 | neuralized homolog | Homo sapiens |
| 1625 | AAH20782 | 643 | 100 | BC020782 glycoprotein hormones, | Homo sapiens |
| | | | | alpha polypeptide | |
| 1625 | J00152 | 643 | 100 | glycoprotein alpha subunit | Homo sapiens |
| 1625 | V00518 | 643 | 100 | chorionic gonadotropin | Homo sapiens |
| 1626 | AAH02722 | 2070 | 100 | BC002722 guanine nucleotide | Homo sapiens |
| | | | | binding protein (G protein), | |
| | | | | alpha stimulating activity | |
| | | | | polypeptide 1 | |
| 1626 | AF493897_1 | 2070 | 100 | AF493897 guanine nucleotide | Homo sapiens |
| | | | | binding protein alpha s long | |
| 1626 | X04408 | 2070 | 100 | alpha-S2 (aa 1-394) | Homo sapiens |
| 1627 | BC033714 | 1724 | 100 | heterogeneous nuclear | Homo sapiens |
| | | | | ribonucleoprotein A1 | |
| 1627 | D86729 | 1724 | 100 | TIS | Mus musculus |
| 1627 | M99167 | 1724 | 100 | RNA binding protein | Mus musculus |
| 1628 | AF072836 | 1827 | 100 | Sox-like transcriptional factor | Homo sapiens |
| 1628 | AF146223_1 | 620 | 100 | AF146223 HMG domain protein HMGX2 | Homo sapiens |
| 1628 | AF288679 | 620 | 100 | smarcel-related protein | Homo sapiens |
| 1629 | AAH21979 | 1360 | 100 | BC021979 ribosomal protein L7a | Homo sapiens |
| 1629 | BC032533 | 1360 | 100 | ribosomal protein L7a | Homo sapiens |
| 1629 | X15013 | 1360 | 100 | ribosomal protein L7a (AA 1-266) | Rattus |
| | | | | | Rattus |
| 1630 | X51538 | 693 | 100 | ribosomal protein S24 (AA 1-133) | Rattus |
| | | | | | Rattus |
| 1630 | X52445 | 693 | 100 | ribosomal protein S24 | Rattus |
| | | | | | norvegicus |
| 1630 | X52658 | 693 | 100 | ribosomal protein S19 (AA 1-133) | Mesocricetus |
| | | | | | auratus |
| 1631 | AAH01503 | 2789 | 100 | BC001503 prosaposin (variant | Homo sapiens |
| | | | | Gaucher disease and variant | |
| | | | | metachromatic leukodystrophy) | |
| 1631 | AAH04275 | 2789 | 100 | BC004275 prosaposin (variant | Homo sapiens |
| | | | | Gaucher disease and variant | |
| | | | | metachromatic leukodystrophy) | |
| 1631 | AAH07612 | 2789 | 100 | BC007612 prosaposin (variant | Homo sapiens |
| | | | | Gaucher disease and variant | |
| | | | | metachromatic leukodystrophy) | |
| 1632 | AAH11776 | 1369 | 100 | BC011776 Similar to tropomyosin 2 | Homo sapiens |
| | | | | (beta) | |
| 1632 | M74817 | 1369 | 100 | tropomyosin-1 | Homo sapiens |
| 1632 | M75165 | 1369 | 100 | tropomyosin | Homo sapiens |
| 1633 | AL023883 | 1173 | 99 | dJ404K8.1 (prolactin) | Homo sapiens |
| 1633 | BC015850 | 1185 | 100 | similar to prolactin precursor | Homo sapiens |
| 1633 | V00566 | 1185 | 100 | reading frame prolactin | Homo sapiens |
| 1634 | AK012601 | 247 | 64 | unnamed protein product | Mus musculus |
| 1634 | AK021409 | 286 | 69 | unnamed protein product | Mus musculus |
| 1634 | AK075801 | 246 | 64 | unnamed protein product | Mus musculus |
| 1635 | AF300943_1 | 9468 | 94 | AF300943 tyrosine phosphatase LAR | Mus musculus |
| 1635 | L11586 | 9484 | 95 | leukocyte common antigen-related | Rattus |
| | | | | phosphatase | norvegicus |
| 1635 | Y00815 | 9953 | 100 | put. LAR preprotein (AA −16 to | Homo sapiens |
| | | | | 1881) | |
| 1636 | AAH16046 | 2739 | 100 | BC016046 ATP synthase, H+ | Homo sapiens |
| | | | | transporting, mitochondrial F1 | |
| | | | | complex, alpha subunit, isoform | |
| | | | | 1, cardiac muscle | |
| 1636 | AAH19310 | 2739 | 100 | BC019310 ATP synthase, H+ | Homo sapiens |
| | | | | transporting, mitochondrial F1 | |
| | | | | complex, alpha subunit, isoform | |
| | | | | 1, cardiac muscle | |
| 1636 | X65460 | 2739 | 100 | ATP synthase alpha subunit | Homo sapiens |
| 1637 | AF237581_1 | 613 | 100 | AF237581 migration inhibitory | Homo sapiens |
| | | | | factor-related protein 14 variant P | |
| 1637 | M26311 | 613 | 100 | cystic fibrosis antigen | Homo sapiens |
| 1637 | X06233 | 613 | 100 | MRP-14 (AA 1-114) | Homo sapiens |
| 1638 | AAH15492 | 523 | 96 | BC015492 Unknown (protein for | Homo sapiens |
| | | | | MGC: 8967) | |
| 1638 | BC032626 | 280 | 64 | TLH29 protein precursor | Homo sapiens |
| 1638 | X67325 | 556 | 100 | ISG12 | Homo sapiens |
| 1639 | L00160 | 2135 | 100 | phosphoglycerate kinase | Homo sapiens |
| 1639 | M11968 | 2135 | 100 | phosphoglycerate kinase | Homo sapiens |
| 1639 | V00572 | 2135 | 100 | coding sequence | Homo sapiens |
| 1640 | AF075575 | 11112 | 100 | dysferlin | Homo sapiens |
| 1640 | AF188290_1 | 10355 | 93 | AF188290 dysferlin | Mus musculus |
| 1640 | AK074104 | 7939 | 98 | FLJ00175 protein | Homo sapiens |
| 1641 | AB052229 | 10414 | 100 | RNA polymerase II largest subunit | Cricetulus |
| | | | | | griseus |
| 1641 | D87294 | 10414 | 100 | RNA polymerase II largest subunit | Cricetulus |
| | | | | | griseus |
| 1641 | X63564 | 10414 | 100 | RNA polymerase II largest subunit | Homo sapiens |
| 1642 | AF070650 | 844 | 100 | F1F0-type ATP synthase subunit d | Homo sapiens |
| 1642 | AF087135 | 844 | 100 | F1FO-type ATPase subunit d | Homo sapiens |
| 1642 | BC038092 | 844 | 100 | ATP synthase, H+ transporting, | Homo sapiens |
| | | | | mitochondrial F0 complex, subunit d | |
| 1643 | U01153 | 4046 | 97 | GRP94 | Canis |
| | | | | | familiaris |
| 1643 | X15187 | 4112 | 100 | precursor polypeptide (AA −21 to | Homo sapiens |
| | | | | 782) | |
| 1643 | Y09136 | 4038 | 97 | gp96/GRP94 | Sus scrofa |
| 1644 | AF297036_1 | 670 | 100 | AF297036 ubiquitin fusion protein | Ophiophagus |
| | | | | | hannah |
| 1644 | BC014772 | 670 | 100 | ubiquitin A-52 residue ribosomal | Mus musculus |
| | | | | protein fusion product 1 | |
| 1644 | M24508 | 670 | 100 | ubiquitin | synthetic |
| | | | | | construct |
| 1645 | AK000385 | 211 | 44 | unnamed protein product | Homo sapiens |
| 1645 | AK097085 | 245 | 51 | unnamed protein product | Homo sapiens |
| 1645 | AK097214 | 201 | 59 | unnamed protein product | Homo sapiens |
| 1646 | AF100956 | 782 | 100 | ribosomal protein subunit S18 | Mus musculus |
| 1646 | AF110520 | 782 | 100 | RPS18 | Mus musculus |
| 1646 | M76763 | 782 | 100 | ribosomal protein | Mus musculus |
| 1647 | BC025077 | 930 | 100 | RIKEN cDNA 2010203J19 gene | Mus musculus |
| 1647 | L05092 | 930 | 100 | ribosomal protein L11 | Homo sapiens |
| 1647 | X62146 | 930 | 100 | ribosomal protein L11 | Rattus |
| | | | | | Rattus |
| 1648 | AB037890 | 6795 | 99 | pre-mRNA splicing factor SF3b 155 kDa | Mus musculus |
| | | | | subunit | |
| 1648 | AF054284 | 6808 | 100 | spliceosomal protein SAP 155 | Homo sapiens |
| 1648 | Y08997 | 6566 | 96 | 146 kDa nuclear protein | Xenopus |
| | | | | | laevis |
| 1649 | AF072097 | 624 | 100 | beta-2 microglobulin | Homo sapiens |
| 1649 | AY007153 | 624 | 100 | similar to Homo sapiens mRNA for | |
| | | | | beta 2-microglobulin with GenBank | |
| | | | | Accession Number AB021288 | |
| 1649 | BC032589 | 624 | 100 | beta-2-microglobulin | Homo sapiens |
| 1650 | AAH17839 | 1602 | 100 | BC017839 caspase 4, apoptosis- | Homo sapiens |
| | | | | related cysteine protease | |
| 1650 | U28977 | 1769 | 100 | Mih1/TX isoform beta | Homo sapiens |
| 1650 | U28978 | 1602 | 100 | Mih1/TX isoform gamma | Homo sapiens |
| 1651 | AAH07967 | 974 | 100 | BC007967 Unknown (protein for | Homo sapiens |
| | | | | MGC: 14460) | |
| 1651 | AAH12149 | 974 | 100 | BC012149 Unknown (protein for | Homo sapiens |
| | | | | MGC: 20365) | |
| 1651 | BC031906 | 974 | 100 | similar to ribosomal protein L9; | Homo sapiens |
| | | | | 60S ribosomal protein L9 | |
| 1652 | BC002145 | 745 | 100 | ribosomal protein S23 | Mus musculus |
| 1652 | D14530 | 745 | 100 | ribosomal protein | Homo sapiens |
| 1652 | X77398 | 745 | 100 | ribosomal protein S23 | Rattus |
| | | | | | norvegicus |
| 1653 | AF227256_1 | 405 | 100 | AF227256 NEDD8 | Bos taurus |
| 1653 | BC004625 | 398 | 98 | neural precursor cell expressed, | Mus musculus |
| | | | | developmentally down-regulated | |
| | | | | gene 8 | |
| 1653 | D23662 | 405 | 100 | ubiquitin-like protein | Homo sapiens |
| 1654 | U46034 | 2076 | 83 | stromelysin 3 | Rattus |
| | | | | | norvegicus |
| 1654 | X57766 | 2679 | 100 | stromelysin-3 precursor | Homo sapiens |
| 1654 | Z12604 | 2164 | 81 | stromelysin-3 | Mus musculus |
| 1655 | AAH01734 | 499 | 100 | BC001734 protein translocation | Homo sapiens |
| | | | | complex beta | |
| 1655 | AL137067 | 499 | 100 | bA13B9.2 (novel protein similar | Homo sapiens |
| | | | | to the beta subunit of transport | |
| | | | | protein SEC61) | |
| 1655 | L25085 | 499 | 100 | Sec61-complex beta-subunit | Homo sapiens |
| 1656 | AF165522_1 | 360 | 100 | AF165522 ras-related GTP-binding | Homo sapiens |
| | | | | protein 4b | |
| 1656 | AF217985_1 | 360 | 100 | AF217985 unknown | Homo sapiens |
| 1656 | AF498935_1 | 360 | 100 | AF498935 small GTP binding | Homo sapiens |
| | | | | protein RAB4B | |
| 1657 | AF006602 | 5436 | 94 | histone deacetylase mHDA1 | Mus musculus |
| 1657 | AF132608_1 | 5784 | 100 | AF132608 histone deacetylase 5 | Homo sapiens |
| 1657 | AF207748_1 | 5431 | 94 | AF207748 histone deacetylase 5 | Mus musculus |
| 1658 | AL050259 | 4009 | 100 | hypothetical protein | Homo sapiens |
| 1658 | BC032681 | 4009 | 100 | RAB2, member RAS oncogene family- | Home sapiens |
| | | | | like | |
| 1658 | Z97184 | 4009 | 100 | cICF0811.4.1 (RAB2, member RAS | Homo sapiens |
| | | | | oncogene family-like, isoform 1) | |
| 1659 | AF151855_1 | 1278 | 100 | AF151855 CGI-97 protein | Homo sapiens |
| 1659 | AK001779 | 1214 | 96 | unnamed protein product | Homo sapiens |
| 1659 | BC003849 | 1200 | 94 | RIKEN cDNA 4733401P19 gene | Mus musculus |
| 1660 | AAH00234 | 1377 | 100 | BC000234 nicotinamide N- | Homo sapiens |
| | | | | methyltransferase | |
| 1660 | U08021 | 1377 | 100 | nicotinamide N-methyltransferase | Homo sapiens |
| 1660 | U20971 | 1377 | 100 | nicotinamide N-methyltransferase | Homo sapiens |
| 1661 | AAH03137 | 1240 | 100 | BC003137 ribosomal protein S3 | Homo sapiens |
| 1661 | BC034149 | 1240 | 100 | ribosomal protein S3 | Homo sapiens |
| 1661 | U14992 | 1240 | 100 | ribosomal protein S3 | Homo sapiens |
| 1662 | AF090891_1 | 3913 | 99 | AF090891 PRO0105 | Homo sapiens |
| 1662 | AF268075_1 | 3913 | 99 | AF268075 TRAF6-binding protein | Homo sapiens |
| | | | | T6BP | |
| 1662 | U33821 | 3924 | 100 | tax1-binding protein TXBP151 | Homo sapiens |
| 1663 | AF186112_1 | 619 | 100 | AF186112 neurokinin B-like | Homo sapiens |
| | | | | protein ZNEUROK1 | |
| 1663 | AF216586_1 | 619 | 100 | AF216586 neurokinin B | Homo sapiens |
| 1663 | BC032145 | 619 | 100 | tachykinin 3 (neuromedin K, | Homo sapiens |
| | | | | neurokinin beta) | |
| 1664 | AAH08405 | 2044 | 90 | BC008405 Unknown (protein for | Homo sapiens |
| | | | | MGC: 14528) | |
| 1664 | M94891 | 2042 | 90 | pregnancy-specific beta-1 | Homo sapiens |
| | | | | glycoprotein | |
| 1664 | U18467 | 2252 | 100 | pregnancy-specific beta 1- | Homo sapiens |
| | | | | glycoprotein 7 precursor | |
| 1665 | AK002989 | 980 | 93 | unnamed protein product | Mus musculus |
| 1665 | D38076 | 1041 | 99 | Ran-BP1 (Ran-binding protein 1) | Homo sapiens |
| 1665 | X83617 | 1057 | 100 | RanBP1 | Homo sapiens |
| 1666 | AF152604 | 3398 | 100 | bromodomain-containing protein | Homo sapiens |
| 1666 | AF213969 | 3369 | 99 | bromodomain protein CELTIX1 | Homo sapiens |
| 1666 | AJ271881 | 3398 | 100 | bromodomain containing protein | Homo sapiens |
| 1667 | AF217973_1 | 329 | 68 | AF217973 unknown | Homo sapiens |
| 1667 | AK000496 | 330 | 66 | unnamed protein product | Homo sapiens |
| 1667 | AK090511 | 352 | 64 | unnamed protein product | Homo sapiens |
| 1668 | AK000917 | 2276 | 100 | unnamed protein product | Homo sapiens |
| 1668 | BC025560 | 2156 | 94 | Similar to hypothetical protein | Mus musculus |
| | | | | FLJ10055 | |
| 1668 | BC039867 | 2259 | 99 | hypothetical protein FLJ10055 | Homo sapiens |
| 1669 | AB039886 | 1071 | 100 | down-regulated in gastric cancer | Homo sapiens |
| 1669 | AY139182 | 995 | 100 | 18 kDa antrum mucosa protein | Homo sapiens |
| 1669 | AY139184 | 995 | 100 | 18 kDa antrum mucosa protein | Homo sapiens |
| 1670 | BC033721 | 3540 | 99 | Unknown (protein for MGC: 45264) | Homo sapiens |
| 1670 | X82157 | 3543 | 100 | hevin | Homo sapiens |
| 1670 | X86693 | 3504 | 99 | Hevin-like protein | Homo sapiens |
| 1671 | M84472 | 1675 | 100 | 17-beta-hydroxysteroid | Homo sapiens |
| | | | | dehydrogenase | |
| 1671 | U34879 | 1675 | 100 | 17-beta-hydroxysteroid | Homo sapiens |
| | | | | dehydrogenase | |
| 1671 | X13440 | 1675 | 100 | 17-HSD protein (AA 1-328) | Homo sapiens |
| 1672 | AAH12612 | 1812 | 99 | BC012612 Similar to cathepsin L | Homo sapiens |
| 1672 | M20496 | 1816 | 100 | preprocathepsin L precursor | Homo sapiens |
| 1672 | X12451 | 1816 | 100 | pro-(cathepsin L) | Homo sapiens |
| 1673 | AAH12355 | 3449 | 100 | BC012355 Similar to solute | Homo sapiens |
| | | | | carrier family 6 | |
| | | | | (neurotransmitter transporter, | |
| | | | | creatine), member 8 | |
| 1673 | U36341 | 3449 | 100 | creatine transporter | Homo sapiens |
| 1673 | Z66539 | 3449 | 100 | creatine transporter | Homo sapiens |
| 1674 | AF267852_1 | 749 | 100 | AF267852 placental protein 13- | Homo sapiens |
| | | | | like protein | |
| 1674 | AF367430 | 571 | 100 | placental protein 13-like | Homo sapiens |
| 1674 | BC022257 | 731 | 98 | Similar to placental protein 13- | Homo sapiens |
| | | | | like protein | |
| 1675 | AB001636 | 4072 | 99 | ATP-dependent RNA helicase #46 | Homo sapiens |
| 1675 | AF279891_1 | 4162 | 100 | AF279891 dead box protein 15 | Homo sapiens |
| 1675 | BC035974 | 4162 | 100 | DEAD/H (Asp-Glu-Ala-Asp/His) box | Homo sapiens |
| | | | | polypeptide 15 | |
| 1676 | AAH17321 | 687 | 100 | BC017321 Unknown (protein for | Homo sapiens |
| | | | | MGC: 29768) | |
| 1676 | BC002079 | 687 | 100 | Similar to ribosomal protein S12 | Mus musculus |
| 1676 | BC018362 | 687 | 100 | Unknown (protein for MGC: 19264) | Mus musculus |
| 1677 | AAH20619 | 1123 | 99 | BC020619 glutathione S- | Homo sapiens |
| | | | | transferase A3 | |
| 1677 | AF508266_1 | 1127 | 100 | AF508266 glutathione transferase | Homo sapiens |
| | | | | A3-3 | |
| 1677 | AL121969 | 1127 | 100 | dJ214M20.1 (glutathione S- | Homo sapiens |
| | | | | transferase A3) | |
| 1678 | AAH00359 | 923 | 100 | BC000359 signal peptidase complex | Homo sapiens |
| | | | | (18 kD) | |
| 1678 | AAH14508 | 923 | 100 | BC014508 Similar to signal | Homo sapiens |
| | | | | peptidase complex (18 kD) | |
| 1678 | AF061737 | 923 | 100 | microsomal signal peptidase | Homo sapiens |
| 1679 | AF179890_1 | 598 | 100 | AF179890 DNA polymerase delta | Homo sapiens |
| | | | | smallest subunit p12 | |
| 1679 | AF515709_1 | 492 | 83 | AF515709 small subunit DNA | Mus musculus |
| | | | | polymerase delta p12 | |
| 1679 | BC028520 | 492 | 83 | Unknown (protein for MGC: 41275) | Mus musculus |
| 1680 | AC005067_2 | 1340 | 73 | AC005067 Supported by Human EST | Homo sapiens |
| | | | | H08032.1 (NID: g872854), mouse EST | |
| | | | | AA870042.1 (NID: g2965487), and | |
| | | | | genscan | |
| 1680 | AF226732_1 | 2286 | 100 | AF226732 NPD007 | Homo sapiens |
| 1680 | BC018360 | 1199 | 68 | Unknown (protein for MGC: 18730) | Mus musculus |
| 1681 | AAH19040 | 897 | 100 | BC019040 Unknown (protein for | Homo sapiens |
| | | | | MGC: 20769) | |
| 1682 | AK023767 | 960 | 100 | unnamed protein product | Homo sapiens |
| 1682 | AL080169 | 979 | 100 | hypothetical protein | Homo sapiens |
| 1682 | BC016647 | 960 | 100 | Unknown (protein for MGC: 16844) | Homo sapiens |
| 1683 | AF309646_1 | 1147 | 88 | AF309646 Rab6-like protein WTH3 | Homo sapiens |
| 1683 | AF498941_1 | 1058 | 100 | AF498941 small GTP binding | Homo sapiens |
| | | | | protein RAB6C | |
| 1683 | AL136727 | 1147 | 88 | hypothetical protein | Homo sapiens |
| 1684 | AC005095_1 | 1373 | 100 | AC005095 pantophysin | Homo sapiens |
| 1684 | S72481 | 1373 | 100 | pantophysin | Homo sapiens |
| 1684 | X68194 | 1373 | 100 | h-Sp1 | Homo sapiens |
| 1685 | D14665 | 3850 | 99 | KIAA0021 protein | Homo sapiens |
| 1685 | U41765 | 3814 | 83 | metalloprotease/disintegrin/cysteine | Mus musculus |
| | | | | rich protein precursor | |
| 1685 | U41766 | 4476 | 100 | metalloprotease/disintegrin/cysteine- | Homo sapiens |
| | | | | rich protein precursor | |
| 1686 | U20770 | 1412 | 100 | cell surface glycoprotein | Homo sapiens |
| 1686 | U67274 | 1412 | 100 | metastasis suppressor | Homo sapiens |
| 1686 | X53795 | 1412 | 100 | inducible nembrane protein | Homo sapiens |
| 1687 | AF517934_1 | 2980 | 100 | AF517934 interleukin 2 receptor, | Homo sapiens |
| | | | | beta | |
| 1687 | AL022314 | 2980 | 100 | dJ1170K4.3 (Interleukin 2 | Homo sapiens |
| | | | | Receptor, Beta (IL-2 Receptor, | |
| | | | | CD122 antigen)) | |
| 1687 | BC025691 | 2980 | 100 | interleukin 2 receptor, beta | Homo sapiens |
| 1688 | AF072242 | 2167 | 100 | methyl-CpG binding protein MBD2 | Homo sapiens |
| 1688 | AF120994 | 2167 | 100 | methyl-CpG binding protein 2 | Homo sapiens |
| 1688 | BC032638 | 2167 | 100 | nethyl-CpG binding domain protein 2 | Homo sapiens |
| 1689 | AAH00486 | 607 | 100 | BC000486 small nuclear | Homo sapiens |
| | | | | ribonucleoprotein D2 polypeptide | |
| | | | | (16.5 kD) | |
| 1689 | AAH01930 | 607 | 100 | BC001930 small nuclear | Homo sapiens |
| | | | | ribonucleoprotein D2 polypeptide | |
| | | | | (16.5 kD) | |
| 1689 | AC00719_1 | 607 | 100 | AC007191 SMD2_HUMAN; SNRNP CORE | Homo sapiens |
| | | | | PROTEIN D2; SM-D2 | |
| 1690 | AAH18180 | 870 | 100 | BC018180 CGI-121 protein | Homo sapiens |
| 1690 | AF151879_1 | 870 | 100 | AF151879 CGI-121 protein | Homo sapiens |
| 1690 | BC029492 | 870 | 100 | CGI-121 protein | Homo sapiens |
| 1691 | U52373 | 4063 | 99 | MNB | Homo sapiens |
| 1691 | U58497 | 4037 | 99 | mp86 | Mus musculus |
| 1691 | X79769 | 4052 | 99 | Dual Specificity Yak1-related | Rattus |
| | | | | Kinase (Dyrk) | norvegicus |

### Example 3

### Initial Determination of Differential Regulation of Polynucleotides of the Invention in Pre-Eclampsia

A plurality of polynucleotide sequences were obtained from cDNA libraries obtained from both normal and pre-eclamptic placental tissues, us9ing SBH sequence signature analysis and Sanger sequencing techniques. The inserts of the library were amplified with PCR using primers specific for the vector sequences which flank the inserts. Clones from cDNA libraries were spotted on nylon membrane filters and screened with oligonucleotide probes (for example, 7-mers) to obtain signature sequences. The clones were clustered into groups of similar or identical sequences. Representative clones were selected for sequencing.

In some cases, the 5′ sequences of the amplified inserts was then deduced using a typical Sanger sequencing protocol. PCR products were purified and subjected to fluorescent dye terminator cycle sequencing. Single pass gel sequencing was done using a 377 Applied Biosystems (ABI) sequencer to obtain the novel nucleic acid sequences. In some cases, RACE (Rapid Amplification of cDNA Ends) was performed to further extend the sequence in the 5′ direction.

Relative expression of the gene may be approximated by a relative abundance of a transcript (number of copies of mRNA) of the gene in question in the cytoplasm of the cells of the tissues in question. Relative abundance of a transcript is calculated as an absolute number of cDNA clones from that transcript in the library created from the total mRNAs of the analyzed tissue over a total number of cDNA clones analyzed per tissue (e.g. 100,000 was the total number of cDNA clones analyzed per tissue. For example, if a gene was represented by 25,000 clones in a given placental library—i.e. it's cDNA cluster comprised 25,000 clones, relative expression of that gene is 25%, since 25% of all clones analyzed belong to that one gene.

Differential expression may be calculated for heterogeneous cDNA clusters which are clusters obtained through clustering of clones from two or more different libraries together. A cluster may be heterogeneous even if it's made up of clones from one library only, provided clustering was done for more than one library. More precisely, heterogeneity is the qualifier of the clustering experiment, rather that the qualifier of the cluster. For example, if two or more libraries were used in the clustering experiment, and a cluster comprises clones from only one of the two libraries, it can still be characterized as heterogeneous, with zero clones from all but one libraries. In this case pairwise comparisons were performed where two libraries were clustered at a time. For given initial conditions (100,000 clones per library, 200,000 total per clustering experiment), clusters of a size of 8 clones or greater were selected for further consideration. Out of these only those showing a differential ratio of clones of 2 times and above were selected as of statistically significant differential expression, thus keeping the confidence level at greater than or equal to 90%. This number measures the probability that the given cluster shows real differential expression. Note that since both libraries in the pair at hand were of the same initial size, therefore, no normalization of the cluster size on the total library size was necessary.

The following table shows the results of this process, indicating which polynucleotides of the invention are designated as up-regulated in pre-eclamptic placental tissues, and which are down-regulated in pre-eclamptic placental tissues.

TABLE 2

| | | | | Expression |
|---|---|---|---|---|
| | | | Expression | in Pre- |
| SEQ ID | | | in Normal | eclamptic |
| NO: | REGULATION | Cluster_ID | Placenta | Placenta |
| 1 | UP | 434 | 7 | 11 |
| 2 | UP | 376 | 108 | 148 |
| 3 | UP | 6623 | 6 | 10 |
| 4 | UP | 1996 | 6 | 15 |
| 5 | UP | 12951 | 17 | 32 |
| 6 | UP | 4704 | 18 | 23 |
| 7 | UP | 3930 | 0 | 8 |
| 8 | UP | 12138 | 25 | 29 |
| 9 | UP | 703 | 126 | 212 |
| 10 | DOWN | 305 | 190 | 180 |
| 11 | DOWN | 8 | 2878 | 1125 |
| 12 | UP | 2498 | 20 | 40 |
| 13 | DOWN | 12700 | 8 | 0 |
| 14 | DOWN | 6779 | 28 | 10 |
| 15 | DOWN | 1037 | 9 | 3 |
| 16 | UP | 8238 | 1 | 8 |
| 17 | UP | 11051 | 10 | 12 |
| 18 | DOWN | 9165 | 22 | 1 |
| 19 | DOWN | 8265 | 9 | 2 |
| 20 | DOWN | 39852 | 8 | 0 |
| 21 | DOWN | 13238 | 16 | 1 |
| 22 | UP | 3184 | 2 | 9 |
| 23 | DOWN | 15366 | 43 | 2 |
| 24 | DOWN | 32470 | 8 | 1 |
| 25 | DOWN | 6195 | 16 | 1 |
| 26 | UP | 69 | 3 | 14 |
| 27 | DOWN | 1550 | 14 | 4 |
| 28 | DOWN | 1544 | 9 | 3 |
| 29 | DOWN | 3129 | 23 | 18 |
| 30 | DOWN | 39826 | 11 | 0 |
| 31 | DOWN | 39825 | 11 | 0 |
| 32 | DOWN | 12237 | 42 | 26 |
| 33 | DOWN | 39614 | 14 | 0 |
| 34 | DOWN | 22223 | 8 | 1 |
| 35 | DOWN | 8274 | 8 | 2 |
| 36 | DOWN | 8993 | 16 | 1 |
| 37 | DOWN | 16337 | 11 | 1 |
| 38 | DOWN | 5959 | 18 | 16 |
| 39 | DOWN | 39367 | 10 | 1 |
| 40 | DOWN | 7336 | 23 | 4 |
| 41 | UP | 1052 | 22 | 73 |
| 42 | DOWN | 6260 | 18 | 1 |
| 43 | DOWN | 7782 | 8 | 0 |
| 44 | DOWN | 14057 | 8 | 0 |
| 45 | UP | 5438 | 14 | 27 |
| 46 | DOWN | 9433 | 15 | 2 |
| 47 | DOWN | 9442 | 18 | 8 |
| 48 | DOWN | 1722 | 36 | 10 |
| 49 | DOWN | 2710 | 31 | 9 |
| 50 | DOWN | 10245 | 14 | 11 |
| 51 | DOWN | 24399 | 15 | 2 |
| 52 | UP | 4592 | 2 | 10 |
| 53 | DOWN | 9466 | 23 | 3 |
| 54 | DOWN | 40893 | 9 | 0 |
| 55 | DOWN | 9639 | 9 | 1 |
| 56 | DOWN | 7143 | 10 | 1 |
| 57 | UP | 571 | 20 | 45 |
| 58 | UP | 396 | 2 | 30 |
| 59 | UP | 1195 | 10 | 17 |
| 60 | UP | 6656 | 2 | 9 |
| 61 | UP | 2970 | 2 | 10 |
| 62 | DOWN | 2929 | 21 | 18 |
| 63 | DOWN | 10556 | 37 | 17 |
| 64 | DOWN | 1033 | 55 | 42 |
| 65 | UP | 115 | 28 | 62 |
| 66 | UP | 1907 | 18 | 22 |
| 67 | UP | 326 | 5 | 32 |
| 68 | UP | 4657 | 12 | 30 |
| 69 | UP | 2228 | 78 | 145 |
| 70 | UP | 3061 | 2 | 8 |
| 71 | UP | 2145 | 55 | 154 |
| 72 | UP | 4072 | 21 | 31 |
| 73 | DOWN | 101 | 77 | 15 |
| 74 | DOWN | 1307 | 19 | 18 |
| 75 | UP | 2602 | 1 | 23 |
| 76 | UP | 2675 | 16 | 55 |
| 77 | UP | 5131 | 28 | 39 |
| 78 | DOWN | 2663 | 20 | 16 |
| 79 | DOWN | 3485 | 56 | 12 |
| 80 | UP | 4189 | 30 | 43 |
| 81 | DOWN | 2880 | 19 | 7 |
| 82 | UP | 3514 | 8 | 35 |
| 83 | UP | 1184 | 24 | 92 |
| 84 | DOWN | 1047 | 20 | 5 |
| 85 | UP | 37 | 33 | 76 |
| 86 | DOWN | 2085 | 17 | 4 |
| 87 | UP | 2655 | 1 | 16 |
| 88 | DOWN | 13007 | 8 | 1 |
| 89 | UP | 920 | 3 | 25 |
| 90 | DOWN | 50 | 71 | 11 |
| 91 | DOWN | 3413 | 97 | 25 |
| 92 | UP | 3610 | 5 | 17 |
| 93 | DOWN | 3705 | 10 | 2 |
| 94 | DOWN | 303 | 18 | 1 |
| 95 | DOWN | 6875 | 14 | 1 |
| 96 | UP | 4791 | 29 | 33 |
| 97 | UP | 1514 | 5 | 10 |
| 98 | UP | 848 | 6 | 29 |
| 99 | DOWN | 1305 | 27 | 7 |
| 100 | UP | 2659 | 29 | 79 |
| 101 | DOWN | 4498 | 37 | 14 |
| 102 | UP | 14168 | 13 | 18 |
| 103 | DOWN | 3391 | 8 | 1 |
| 104 | UP | 3013 | 10 | 15 |
| 105 | UP | 1182 | 2 | 32 |
| 106 | DOWN | 1177 | 103 | 7 |
| 107 | UP | 5258 | 4 | 11 |
| 108 | DOWN | 1739 | 20 | 1 |
| 109 | UP | 95 | 10 | 30 |
| 110 | UP | 664 | 19 | 20 |
| 111 | UP | 12341 | 1 | 8 |
| 112 | UP | 99 | 14 | 119 |
| 113 | UP | 168 | 23 | 53 |
| 114 | DOWN | 1492 | 32 | 11 |
| 115 | DOWN | 769 | 26 | 23 |
| 116 | UP | 87 | 6 | 16 |
| 117 | UP | 1314 | 1 | 27 |
| 118 | DOWN | 1147 | 126 | 100 |
| 119 | UP | 4205 | 6 | 23 |
| 120 | DOWN | 2994 | 19 | 9 |
| 121 | UP | 843 | 21 | 36 |
| 122 | UP | 1220 | 4 | 12 |
| 123 | DOWN | 482 | 23 | 3 |
| 124 | UP | 1061 | 2 | 9 |
| 125 | UP | 2205 | 10 | 42 |
| 126 | DOWN | 1730 | 21 | 18 |
| 127 | DOWN | 5983 | 51 | 11 |
| 128 | UP | 10559 | 23 | 94 |
| 129 | DOWN | 2158 | 28 | 5 |
| 130 | UP | 252 | 13 | 135 |
| 131 | DOWN | 4956 | 38 | 27 |
| 132 | DOWN | 7031 | 17 | 2 |
| 133 | DOWN | 1670 | 9 | 1 |
| 134 | DOWN | 30049 | 23 | 2 |
| 135 | DOWN | 27955 | 8 | 0 |
| 136 | DOWN | 7679 | 8 | 1 |
| 137 | UP | 2741 | 9 | 15 |
| 138 | DOWN | 7542 | 11 | 4 |
| 139 | UP | 8179 | 6 | 9 |
| 140 | UP | 7089 | 4 | 14 |
| 141 | UP | 6968 | 13 | 17 |
| 142 | UP | 7166 | 3 | 34 |
| 143 | UP | 7378 | 9 | 34 |
| 144 | DOWN | 6954 | 25 | 5 |
| 145 | DOWN | 3051 | 16 | 1 |
| 146 | DOWN | 5485 | 14 | 5 |
| 147 | UP | 5128 | 6 | 25 |
| 148 | UP | 6749 | 4 | 18 |
| 149 | DOWN | 5070 | 15 | 11 |
| 150 | UP | 1059 | 8 | 10 |
| 151 | UP | 4885 | 18 | 21 |
| 152 | UP | 4643 | 30 | 100 |
| 153 | DOWN | 5058 | 15 | 9 |
| 154 | UP | 5980 | 5 | 21 |
| 155 | UP | 2089 | 6 | 11 |
| 156 | DOWN | 5490 | 28 | 22 |
| 157 | UP | 4821 | 52 | 95 |
| 158 | UP | 61 | 13 | 27 |
| 159 | UP | 4710 | 7 | 37 |
| 160 | DOWN | 8083 | 8 | 1 |
| 161 | UP | 4878 | 9 | 43 |
| 162 | DOWN | 1857 | 25 | 21 |
| 163 | DOWN | 5346 | 11 | 3 |
| 164 | UP | 5124 | 21 | 55 |
| 165 | UP | 4804 | 41 | 123 |
| 166 | DOWN | 6555 | 10 | 3 |
| 167 | DOWN | 6869 | 8 | 2 |
| 168 | DOWN | 7961 | 13 | 8 |
| 169 | DOWN | 6854 | 58 | 13 |
| 170 | UP | 13272 | 3 | 15 |
| 171 | UP | 4796 | 66 | 85 |
| 172 | UP | 258 | 12 | 13 |
| 173 | UP | 5334 | 1 | 9 |
| 174 | UP | 10290 | 0 | 24 |
| 175 | DOWN | 5245 | 37 | 5 |
| 176 | DOWN | 12521 | 8 | 2 |
| 177 | UP | 1382 | 79 | 148 |
| 178 | DOWN | 12566 | 9 | 0 |
| 179 | UP | 6354 | 7 | 10 |
| 180 | UP | 4945 | 14 | 65 |
| 181 | DOWN | 5491 | 72 | 25 |
| 182 | DOWN | 10400 | 22 | 2 |
| 183 | UP | 5702 | 7 | 25 |
| 184 | DOWN | 3050 | 22 | 17 |
| 185 | DOWN | 6201 | 20 | 9 |
| 186 | UP | 4884 | 3 | 27 |
| 187 | DOWN | 7725 | 8 | 1 |
| 188 | UP | 5988 | 43 | 71 |
| 189 | UP | 11831 | 3 | 16 |
| 190 | DOWN | 4855 | 16 | 3 |
| 191 | UP | 786 | 25 | 27 |
| 192 | UP | 5263 | 4 | 19 |
| 193 | UP | 5556 | 16 | 20 |
| 194 | UP | 7758 | 1 | 8 |
| 195 | UP | 9188 | 1 | 17 |
| 196 | DOWN | 4698 | 18 | 16 |
| 197 | UP | 5654 | 0 | 8 |
| 198 | UP | 4937 | 3 | 10 |
| 199 | UP | 5890 | 2 | 18 |
| 200 | UP | 3983 | 5 | 46 |
| 201 | DOWN | 7240 | 45 | 19 |
| 202 | UP | 5555 | 3 | 8 |
| 203 | UP | 5276 | 4 | 9 |
| 204 | DOWN | 6687 | 49 | 22 |
| 205 | UP | 3547 | 31 | 93 |
| 206 | UP | 3513 | 14 | 54 |
| 207 | DOWN | 10446 | 8 | 1 |
| 208 | UP | 10774 | 8 | 13 |
| 209 | UP | 10793 | 1 | 7 |
| 210 | DOWN | 10988 | 26 | 1 |
| 211 | UP | 4366 | 1 | 8 |
| 212 | DOWN | 6243 | 119 | 96 |
| 213 | DOWN | 7248 | 8 | 0 |
| 214 | UP | 8984 | 2 | 10 |
| 215 | UP | 561 | 5 | 26 |
| 216 | DOWN | 10657 | 13 | 11 |
| 217 | UP | 9847 | 5 | 12 |
| 218 | UP | 10184 | 9 | 14 |
| 219 | UP | 5279 | 6 | 16 |
| 220 | UP | 12109 | 8 | 22 |
| 221 | UP | 3955 | 7 | 16 |
| 222 | UP | 8885 | 2 | 10 |
| 223 | UP | 10365 | 2 | 21 |
| 224 | UP | 1178 | 5 | 30 |
| 225 | UP | 4066 | 5 | 17 |
| 226 | UP | 1760 | 11 | 11 |
| 227 | UP | 5969 | 12 | 45 |
| 228 | DOWN | 4883 | 8 | 1 |
| 229 | UP | 5163 | 17 | 38 |
| 230 | UP | 10739 | 3 | 8 |
| 231 | DOWN | 12507 | 52 | 17 |
| 232 | DOWN | 1812 | 35 | 30 |
| 233 | UP | 134 | 0 | 18 |
| 234 | DOWN | 3197 | 10 | 2 |
| 235 | UP | 9077 | 8 | 12 |
| 236 | DOWN | 10016 | 21 | 17 |
| 237 | UP | 3788 | 3 | 11 |
| 238 | DOWN | 10011 | 8 | 1 |
| 239 | UP | 9599 | 3 | 9 |
| 240 | DOWN | 6566 | 64 | 1 |
| 241 | DOWN | 9865 | 19 | 1 |
| 242 | UP | 10885 | 17 | 26 |
| 243 | UP | 12292 | 5 | 22 |
| 244 | DOWN | 12390 | 45 | 22 |
| 245 | DOWN | 13573 | 49 | 4 |
| 246 | DOWN | 1203 | 35 | 4 |
| 247 | UP | 9768 | 19 | 33 |
| 248 | UP | 6552 | 3 | 12 |
| 249 | DOWN | 10363 | 9 | 4 |
| 250 | UP | 649 | 15 | 27 |
| 251 | UP | 12635 | 0 | 10 |
| 252 | DOWN | 464 | 17 | 6 |
| 253 | DOWN | 12809 | 21 | 2 |
| 254 | UP | 2868 | 11 | 34 |
| 255 | DOWN | 6423 | 136 | 7 |
| 256 | UP | 8249 | 6 | 13 |
| 257 | DOWN | 4665 | 24 | 14 |
| 258 | DOWN | 3521 | 18 | 17 |
| 259 | UP | 3001 | 18 | 25 |
| 260 | DOWN | 11740 | 8 | 2 |
| 261 | UP | 13122 | 5 | 16 |
| 262 | UP | 2177 | 48 | 72 |
| 263 | UP | 8444 | 11 | 24 |
| 264 | DOWN | 2187 | 57 | 5 |
| 265 | UP | 6962 | 61 | 148 |
| 266 | DOWN | 12538 | 7 | 2 |
| 267 | UP | 443 | 44 | 67 |
| 268 | DOWN | 13257 | 14 | 5 |
| 269 | DOWN | 13549 | 19 | 0 |
| 270 | DOWN | 4087 | 11 | 2 |
| 271 | UP | 45 | 3 | 20 |
| 272 | DOWN | 12290 | 13 | 9 |
| 273 | UP | 3570 | 11 | 49 |
| 274 | UP | 7142 | 2 | 9 |
| 275 | DOWN | 13596 | 37 | 12 |
| 276 | UP | 1975 | 34 | 251 |
| 277 | DOWN | 901 | 16 | 6 |
| 278 | UP | 5652 | 77 | 107 |
| 279 | DOWN | 10591 | 11 | 2 |
| 280 | DOWN | 11910 | 9 | 2 |
| 281 | UP | 12090 | 6 | 26 |
| 282 | DOWN | 4495 | 167 | 33 |
| 283 | UP | 3739 | 5 | 32 |
| 284 | UP | 5064 | 2 | 18 |
| 285 | DOWN | 10511 | 23 | 2 |
| 286 | UP | 1750 | 2 | 9 |
| 287 | DOWN | 2968 | 16 | 4 |
| 288 | UP | 12172 | 7 | 31 |
| 289 | UP | 11685 | 9 | 13 |
| 290 | UP | 12822 | 4 | 14 |
| 291 | UP | 4732 | 9 | 12 |
| 292 | UP | 12971 | 1 | 8 |
| 293 | UP | 12180 | 4 | 8 |
| 294 | UP | 6618 | 8 | 13 |
| 295 | DOWN | 12247 | 23 | 4 |
| 296 | UP | 9086 | 10 | 23 |
| 297 | UP | 11765 | 20 | 33 |
| 298 | UP | 12767 | 2 | 38 |
| 299 | DOWN | 2136 | 13 | 12 |
| 300 | UP | 1110 | 1 | 8 |
| 301 | UP | 10099 | 26 | 75 |
| 302 | DOWN | 4800 | 10 | 2 |
| 303 | DOWN | 13652 | 8 | 1 |
| 304 | UP | 4762 | 3 | 13 |
| 305 | UP | 1519 | 0 | 8 |
| 306 | UP | 223 | 1 | 17 |
| 307 | UP | 13680 | 1 | 8 |
| 308 | UP | 11884 | 1 | 8 |
| 309 | DOWN | 1681 | 16 | 13 |
| 310 | UP | 11800 | 16 | 36 |
| 311 | UP | 6565 | 111 | 125 |
| 312 | UP | 13555 | 9 | 11 |
| 313 | DOWN | 7878 | 15 | 4 |
| 314 | DOWN | 11630 | 12 | 3 |
| 315 | DOWN | 8596 | 29 | 12 |
| 316 | DOWN | 8005 | 17 | 8 |
| 317 | DOWN | 5037 | 29 | 12 |
| 318 | UP | 644 | 29 | 38 |
| 319 | UP | 7190 | 1 | 7 |
| 320 | UP | 12055 | 9 | 26 |
| 321 | DOWN | 4714 | 44 | 9 |
| 322 | UP | 4330 | 2 | 10 |
| 323 | UP | 6773 | 14 | 53 |
| 324 | DOWN | 9057 | 81 | 26 |
| 325 | DOWN | 2907 | 9 | 1 |
| 326 | UP | 2910 | 3 | 17 |
| 327 | DOWN | 9989 | 10 | 3 |
| 328 | UP | 1713 | 2 | 8 |
| 329 | UP | 3618 | 23 | 67 |
| 330 | UP | 2252 | 6 | 17 |
| 331 | DOWN | 12013 | 13 | 2 |
| 332 | UP | 6673 | 24 | 38 |
| 333 | DOWN | 2091 | 64 | 11 |
| 334 | DOWN | 11539 | 43 | 28 |
| 335 | DOWN | 14305 | 8 | 1 |
| 336 | UP | 8835 | 14 | 53 |
| 337 | DOWN | 12824 | 15 | 5 |
| 338 | DOWN | 13661 | 14 | 5 |
| 339 | UP | 8290 | 22 | 24 |
| 340 | DOWN | 12559 | 8 | 1 |
| 341 | UP | 10192 | 21 | 34 |
| 342 | UP | 8540 | 3 | 17 |
| 343 | DOWN | 563 | 18 | 8 |
| 344 | UP | 5319 | 3 | 11 |
| 345 | DOWN | 1131 | 19 | 13 |
| 346 | DOWN | 10034 | 25 | 18 |
| 347 | UP | 10644 | 0 | 8 |
| 348 | DOWN | 6564 | 15 | 10 |
| 349 | UP | 656 | 5 | 10 |
| 350 | DOWN | 13465 | 16 | 11 |
| 351 | DOWN | 13564 | 20 | 8 |
| 352 | UP | 9110 | 26 | 61 |
| 353 | DOWN | 6594 | 80 | 42 |
| 354 | UP | 1637 | 13 | 44 |
| 355 | UP | 13262 | 3 | 26 |
| 356 | UP | 484 | 16 | 69 |
| 357 | DOWN | 10314 | 63 | 11 |
| 358 | DOWN | 9880 | 8 | 0 |
| 359 | DOWN | 10993 | 17 | 4 |
| 360 | UP | 5788 | 7 | 18 |
| 361 | DOWN | 9892 | 109 | 54 |
| 362 | UP | 1602 | 38 | 83 |
| 363 | UP | 1191 | 21 | 44 |
| 364 | DOWN | 128 | 125 | 47 |
| 365 | UP | 239 | 166 | 949 |
| 366 | DOWN | 1202 | 111 | 90 |
| 367 | DOWN | 3724 | 22 | 3 |
| 368 | UP | 1080 | 1 | 9 |
| 369 | UP | 2376 | 33 | 94 |
| 370 | UP | 190 | 508 | 774 |
| 371 | UP | 3866 | 11 | 65 |
| 372 | DOWN | 27995 | 12 | 1 |
| 373 | UP | 4199 | 1 | 7 |
| 374 | UP | 311 | 2 | 21 |
| 375 | DOWN | 3585 | 20 | 14 |
| 376 | DOWN | 6583 | 9 | 2 |
| 377 | UP | 5646 | 9 | 33 |
| 378 | UP | 4746 | 1 | 27 |
| 379 | DOWN | 8891 | 33 | 2 |
| 380 | UP | 13605 | 8 | 13 |
| 381 | UP | 4479 | 16 | 118 |
| 382 | UP | 7523 | 2 | 9 |
| 383 | UP | 7575 | 1 | 16 |
| 384 | DOWN | 4842 | 18 | 3 |
| 385 | UP | 2071 | 31 | 72 |
| 386 | UP | 6828 | 1 | 24 |
| 387 | UP | 9973 | 18 | 38 |
| 388 | DOWN | 2141 | 104 | 61 |
| 389 | UP | 9003 | 10 | 16 |
| 390 | DOWN | 12691 | 25 | 2 |
| 391 | DOWN | 13237 | 19 | 6 |
| 392 | UP | 220 | 1 | 8 |
| 393 | UP | 5371 | 10 | 33 |
| 394 | UP | 12723 | 25 | 39 |
| 395 | DOWN | 9954 | 31 | 13 |
| 396 | UP | 1461 | 48 | 51 |
| 397 | DOWN | 10742 | 10 | 2 |
| 398 | UP | 5189 | 23 | 54 |
| 399 | UP | 2862 | 21 | 97 |
| 400 | UP | 12888 | 1 | 9 |
| 401 | UP | 8225 | 4 | 21 |
| 402 | DOWN | 5829 | 16 | 5 |
| 403 | DOWN | 1238 | 36 | 31 |
| 404 | UP | 2234 | 50 | 55 |
| 405 | UP | 10464 | 5 | 13 |
| 406 | UP | 1439 | 16 | 19 |
| 407 | DOWN | 6353 | 39 | 17 |
| 408 | UP | 8356 | 8 | 12 |
| 409 | UP | 12863 | 3 | 18 |
| 410 | UP | 5391 | 14 | 17 |
| 411 | DOWN | 660 | 29 | 3 |
| 412 | UP | 12828 | 3 | 19 |
| 413 | DOWN | 6367 | 16 | 1 |
| 414 | UP | 7641 | 16 | 30 |
| 415 | UP | 10503 | 2 | 10 |
| 416 | UP | 3687 | 10 | 17 |
| 417 | DOWN | 5524 | 37 | 15 |
| 418 | DOWN | 15 | 2577 | 450 |
| 419 | DOWN | 6087 | 11 | 7 |
| 420 | DOWN | 337 | 56 | 13 |
| 421 | UP | 38 | 11 | 24 |
| 422 | UP | 1940 | 55 | 67 |
| 423 | DOWN | 1002 | 22 | 7 |
| 424 | DOWN | 1922 | 22 | 10 |
| 425 | DOWN | 6192 | 58 | 24 |
| 426 | UP | 2041 | 33 | 60 |
| 427 | DOWN | 9351 | 31 | 1 |
| 428 | UP | 1758 | 10 | 16 |
| 429 | UP | 2954 | 8 | 35 |
| 430 | UP | 4097 | 34 | 66 |
| 431 | DOWN | 487 | 82 | 7 |
| 432 | DOWN | 6228 | 25 | 8 |
| 433 | UP | 235 | 45 | 99 |
| 434 | UP | 690 | 24 | 48 |
| 435 | UP | 1362 | 4 | 28 |
| 436 | UP | 1978 | 48 | 93 |
| 437 | UP | 691 | 2 | 8 |
| 438 | DOWN | 3805 | 11 | 1 |
| 439 | UP | 3639 | 98 | 115 |
| 440 | DOWN | 6919 | 8 | 1 |
| 441 | DOWN | 585 | 84 | 35 |
| 442 | DOWN | 4436 | 18 | 1 |
| 443 | UP | 8923 | 27 | 31 |
| 444 | UP | 24 | 226 | 377 |
| 445 | UP | 1211 | 67 | 106 |
| 446 | DOWN | 5473 | 8 | 0 |
| 447 | UP | 380 | 218 | 454 |
| 448 | DOWN | 2785 | 13 | 6 |
| 449 | UP | 6090 | 16 | 16 |
| 450 | UP | 177 | 23 | 34 |
| 451 | DOWN | 3632 | 25 | 11 |
| 452 | UP | 6400 | 2 | 10 |
| 453 | DOWN | 1660 | 49 | 13 |
| 454 | UP | 1934 | 24 | 95 |
| 455 | UP | 2680 | 1 | 12 |
| 456 | UP | 136 | 10 | 91 |
| 457 | DOWN | 9445 | 306 | 47 |
| 458 | DOWN | 4763 | 82 | 43 |
| 459 | DOWN | 1068 | 40 | 33 |
| 460 | DOWN | 1797 | 32 | 19 |
| 461 | DOWN | 8275 | 56 | 2 |
| 462 | UP | 265 | 0 | 8 |
| 463 | UP | 6547 | 2 | 11 |
| 464 | UP | 9244 | 12 | 36 |
| 465 | UP | 5676 | 9 | 15 |
| 466 | UP | 96 | 43 | 67 |
| 467 | UP | 6047 | 3 | 26 |
| 468 | UP | 3559 | 1 | 9 |
| 469 | UP | 6182 | 4 | 9 |
| 470 | DOWN | 8377 | 139 | 58 |
| 471 | UP | 1265 | 13 | 48 |
| 472 | DOWN | 12747 | 15 | 5 |
| 473 | DOWN | 14061 | 21 | 5 |
| 474 | UP | 365 | 89 | 367 |
| 475 | UP | 2363 | 68 | 88 |
| 476 | UP | 10195 | 14 | 18 |
| 477 | DOWN | 4520 | 42 | 12 |
| 478 | DOWN | 12435 | 12 | 3 |
| 479 | UP | 884 | 9 | 22 |
| 480 | UP | 4138 | 5 | 18 |
| 481 | UP | 1154 | 3 | 19 |
| 482 | UP | 2778 | 47 | 71 |
| 483 | DOWN | 852 | 16 | 15 |
| 484 | DOWN | 7188 | 110 | 20 |
| 485 | DOWN | 12168 | 9 | 1 |
| 486 | UP | 11591 | 1 | 15 |
| 487 | UP | 10039 | 2 | 12 |
| 488 | UP | 5194 | 11 | 32 |
| 489 | UP | 10061 | 1 | 7 |
| 490 | DOWN | 1994 | 30 | 20 |
| 491 | UP | 11941 | 8 | 78 |
| 492 | DOWN | 5733 | 12 | 8 |
| 493 | DOWN | 1299 | 12 | 4 |
| 494 | UP | 86 | 9 | 25 |
| 495 | UP | 292 | 123 | 155 |
| 496 | UP | 3644 | 1 | 19 |
| 497 | DOWN | 11458 | 19 | 13 |
| 498 | UP | 12641 | 1 | 7 |
| 499 | DOWN | 11674 | 75 | 9 |
| 500 | UP | 3089 | 0 | 8 |
| 501 | DOWN | 8552 | 9 | 3 |
| 502 | DOWN | 4808 | 25 | 1 |
| 503 | UP | 6540 | 16 | 33 |
| 504 | UP | 3966 | 13 | 33 |
| 505 | DOWN | 886 | 10 | 2 |
| 506 | UP | 3167 | 23 | 25 |
| 507 | UP | 1246 | 4 | 12 |
| 508 | UP | 3018 | 23 | 25 |
| 509 | UP | 506 | 2 | 8 |
| 510 | UP | 4683 | 4 | 20 |
| 511 | UP | 1165 | 41 | 78 |
| 512 | DOWN | 8148 | 8 | 0 |
| 513 | UP | 5746 | 4 | 9 |
| 514 | DOWN | 865 | 83 | 74 |
| 515 | UP | 3235 | 18 | 37 |
| 516 | DOWN | 5271 | 61 | 5 |
| 517 | DOWN | 4605 | 38 | 22 |
| 518 | UP | 517 | 64 | 95 |
| 519 | UP | 1341 | 23 | 35 |
| 520 | DOWN | 8858 | 32 | 13 |
| 521 | DOWN | 8205 | 10 | 1 |
| 522 | UP | 6977 | 12 | 22 |
| 523 | DOWN | 12678 | 15 | 9 |
| 524 | DOWN | 931 | 24 | 8 |
| 525 | DOWN | 12947 | 38 | 11 |
| 526 | UP | 8955 | 17 | 39 |
| 527 | DOWN | 9418 | 8 | 1 |
| 528 | UP | 1113 | 29 | 129 |
| 529 | DOWN | 3617 | 25 | 23 |
| 530 | UP | 5243 | 10 | 14 |
| 531 | UP | 4246 | 22 | 49 |
| 532 | UP | 8909 | 5 | 31 |
| 533 | UP | 1213 | 18 | 49 |
| 534 | UP | 13318 | 2 | 10 |
| 535 | UP | 574 | 1 | 7 |
| 536 | DOWN | 9166 | 9 | 1 |
| 537 | UP | 6211 | 5 | 37 |
| 538 | UP | 2771 | 1 | 15 |
| 539 | UP | 341 | 38 | 74 |
| 540 | DOWN | 4688 | 204 | 84 |
| 541 | UP | 3219 | 1 | 7 |
| 542 | UP | 6056 | 14 | 32 |
| 543 | DOWN | 6739 | 97 | 65 |
| 544 | UP | 629 | 172 | 487 |
| 545 | UP | 3239 | 62 | 106 |
| 546 | DOWN | 4958 | 1096 | 5 |
| 547 | DOWN | 2735 | 14 | 3 |
| 548 | UP | 6224 | 4 | 40 |
| 549 | UP | 3142 | 6 | 23 |
| 550 | DOWN | 2098 | 57 | 34 |
| 551 | UP | 1554 | 25 | 128 |
| 552 | UP | 2424 | 2 | 13 |
| 553 | UP | 127 | 2 | 8 |
| 554 | DOWN | 212 | 28 | 3 |
| 555 | UP | 4776 | 4 | 10 |
| 556 | DOWN | 327 | 18 | 1 |
| 557 | UP | 12520 | 1 | 10 |
| 558 | DOWN | 4604 | 11 | 4 |
| 559 | DOWN | 9810 | 16 | 1 |
| 560 | UP | 360 | 0 | 11 |
| 561 | DOWN | 10867 | 9 | 1 |
| 562 | UP | 1912 | 97 | 214 |
| 563 | DOWN | 751 | 123 | 120 |
| 564 | UP | 175 | 33 | 250 |
| 565 | DOWN | 5049 | 16 | 6 |
| 566 | UP | 65 | 115 | 329 |
| 567 | UP | 2609 | 17 | 42 |
| 568 | UP | 11967 | 5 | 16 |
| 569 | UP | 9230 | 12 | 30 |
| 570 | UP | 3946 | 33 | 96 |
| 571 | UP | 752 | 21 | 58 |
| 572 | UP | 4767 | 12 | 127 |
| 573 | DOWN | 9958 | 8 | 1 |
| 574 | UP | 1133 | 91 | 100 |
| 575 | UP | 5087 | 1 | 8 |
| 576 | UP | 12332 | 9 | 12 |
| 577 | UP | 383 | 11 | 45 |
| 578 | UP | 10259 | 4 | 18 |
| 579 | DOWN | 524 | 51 | 21 |
| 580 | DOWN | 11813 | 46 | 9 |
| 581 | DOWN | 6060 | 8 | 1 |
| 582 | DOWN | 923 | 196 | 162 |
| 583 | DOWN | 6619 | 8 | 1 |
| 584 | DOWN | 4150 | 16 | 10 |
| 585 | UP | 4185 | 25 | 30 |
| 586 | UP | 3764 | 16 | 19 |
| 587 | DOWN | 11835 | 47 | 8 |
| 588 | UP | 222 | 16 | 39 |
| 589 | UP | 393 | 7 | 37 |
| 590 | UP | 4352 | 26 | 90 |
| 591 | UP | 5384 | 30 | 50 |
| 592 | DOWN | 3019 | 20 | 16 |
| 593 | UP | 5001 | 4 | 17 |
| 594 | UP | 3370 | 14 | 31 |
| 595 | DOWN | 7069 | 24 | 19 |
| 596 | DOWN | 5126 | 59 | 33 |
| 597 | UP | 3532 | 2 | 10 |
| 598 | UP | 12 | 1645 | 2225 |
| 599 | UP | 193 | 21 | 22 |
| 600 | UP | 8086 | 20 | 24 |
| 601 | UP | 2586 | 47 | 299 |
| 602 | UP | 8780 | 23 | 72 |
| 603 | DOWN | 13928 | 45 | 22 |
| 604 | DOWN | 11269 | 24 | 1 |
| 605 | DOWN | 7070 | 58 | 37 |
| 606 | UP | 1557 | 42 | 97 |
| 607 | UP | 11999 | 5 | 19 |
| 608 | UP | 11661 | 22 | 33 |
| 609 | DOWN | 70 | 650 | 611 |
| 610 | DOWN | 498 | 20 | 16 |
| 611 | DOWN | 917 | 16 | 13 |
| 612 | UP | 8974 | 8 | 11 |
| 613 | UP | 2067 | 105 | 268 |
| 614 | UP | 2443 | 8 | 17 |
| 615 | UP | 91 | 53 | 98 |
| 616 | UP | 1585 | 9 | 34 |
| 617 | DOWN | 1283 | 14 | 10 |
| 618 | UP | 1977 | 59 | 112 |
| 619 | DOWN | 6535 | 8 | 1 |
| 620 | UP | 9417 | 19 | 32 |
| 621 | DOWN | 8959 | 18 | 17 |
| 622 | UP | 8095 | 3 | 22 |
| 623 | DOWN | 10468 | 8 | 1 |
| 624 | UP | 6664 | 13 | 18 |
| 625 | DOWN | 2971 | 15 | 4 |
| 626 | UP | 4386 | 19 | 50 |
| 627 | DOWN | 3843 | 321 | 16 |
| 628 | UP | 6223 | 2 | 9 |
| 629 | DOWN | 9914 | 12 | 4 |
| 630 | DOWN | 3261 | 8 | 1 |
| 631 | UP | 12511 | 12 | 48 |
| 632 | UP | 1883 | 2 | 8 |
| 633 | UP | 1918 | 3 | 32 |
| 634 | DOWN | 9695 | 63 | 4 |
| 635 | UP | 8882 | 10 | 28 |
| 636 | DOWN | 2315 | 300 | 277 |
| 637 | UP | 1286 | 33 | 46 |
| 638 | UP | 6004 | 81 | 149 |
| 639 | UP | 2236 | 44 | 83 |
| 640 | UP | 12400 | 2 | 9 |
| 641 | DOWN | 6716 | 130 | 7 |
| 642 | UP | 3715 | 9 | 9 |
| 643 | DOWN | 6931 | 20 | 14 |
| 644 | DOWN | 5278 | 82 | 13 |
| 645 | UP | 7811 | 1 | 15 |
| 646 | DOWN | 3783 | 124 | 76 |
| 647 | DOWN | 724 | 42 | 34 |
| 648 | UP | 13530 | 1 | 7 |
| 649 | UP | 666 | 1 | 9 |
| 650 | UP | 741 | 3 | 23 |
| 651 | DOWN | 7002 | 402 | 1 |
| 652 | DOWN | 9722 | 14 | 7 |
| 653 | DOWN | 8831 | 11 | 1 |
| 654 | UP | 100 | 25 | 63 |
| 655 | UP | 185 | 101 | 105 |
| 656 | UP | 13050 | 2 | 8 |
| 657 | UP | 1215 | 161 | 339 |
| 658 | UP | 1493 | 27 | 42 |
| 659 | UP | 2647 | 0 | 11 |
| 660 | UP | 11865 | 2 | 9 |
| 661 | UP | 335 | 2 | 29 |
| 662 | DOWN | 9974 | 9 | 1 |
| 663 | UP | 4325 | 0 | 8 |
| 664 | UP | 5859 | 9 | 25 |
| 665 | DOWN | 10755 | 7 | 2 |
| 666 | DOWN | 8227 | 27 | 7 |
| 667 | UP | 3716 | 7 | 46 |
| 668 | UP | 3304 | 5 | 24 |
| 669 | UP | 3045 | 3 | 12 |
| 670 | DOWN | 2017 | 13 | 2 |
| 671 | DOWN | 12994 | 8 | 0 |
| 672 | UP | 2386 | 54 | 111 |
| 673 | DOWN | 8884 | 98 | 11 |
| 674 | UP | 3949 | 30 | 58 |
| 675 | DOWN | 11971 | 21 | 19 |
| 676 | DOWN | 8080 | 8 | 1 |
| 677 | UP | 1932 | 37 | 71 |
| 678 | UP | 1735 | 1 | 8 |
| 679 | UP | 10004 | 5 | 14 |
| 680 | UP | 2313 | 2 | 8 |
| 681 | UP | 413 | 12 | 25 |
| 682 | UP | 824 | 51 | 102 |
| 683 | UP | 5494 | 87 | 133 |
| 684 | UP | 11842 | 11 | 49 |
| 685 | UP | 888 | 17 | 38 |
| 686 | DOWN | 9904 | 27 | 13 |
| 687 | UP | 6 | 141 | 4273 |
| 688 | DOWN | 4933 | 180 | 33 |
| 689 | DOWN | 4859 | 12 | 5 |
| 690 | DOWN | 7568 | 55 | 35 |
| 691 | DOWN | 10505 | 44 | 27 |
| 692 | DOWN | 2383 | 34 | 20 |
| 693 | UP | 182 | 2065 | 3955 |
| 694 | DOWN | 6129 | 143 | 40 |
| 695 | UP | 10187 | 9 | 21 |
| 696 | UP | 3 | 647 | 666 |
| 697 | UP | 3315 | 8 | 10 |
| 698 | DOWN | 12014 | 17 | 4 |
| 699 | UP | 1 | 2954 | 7028 |
| 700 | UP | 1206 | 16 | 233 |
| 701 | UP | 2821 | 30 | 83 |
| 702 | UP | 11882 | 2 | 9 |
| 703 | UP | 608 | 89 | 201 |
| 704 | DOWN | 2261 | 20 | 2 |
| 705 | UP | 7728 | 25 | 26 |
| 706 | UP | 5 | 3558 | 3991 |
| 707 | DOWN | 5075 | 66 | 0 |
| 708 | DOWN | 1709 | 8 | 0 |
| 709 | UP | 2058 | 10 | 19 |
| 710 | UP | 8913 | 14 | 33 |
| 711 | DOWN | 2605 | 23 | 3 |
| 712 | DOWN | 6388 | 8 | 0 |
| 713 | UP | 398 | 29 | 125 |
| 714 | UP | 146 | 17 | 54 |
| 715 | UP | 4 | 257 | 812 |
| 716 | UP | 5626 | 14 | 21 |
| 717 | UP | 2099 | 31 | 123 |
| 718 | UP | 9142 | 24 | 25 |
| 719 | UP | 11744 | 14 | 29 |
| 720 | UP | 123 | 198 | 1192 |
| 721 | UP | 2775 | 33 | 83 |
| 722 | UP | 2949 | 2 | 9 |
| 723 | UP | 3244 | 53 | 97 |
| 724 | UP | 153 | 3 | 16 |
| 725 | UP | 1034 | 11 | 48 |
| 726 | DOWN | 3515 | 14 | 6 |
| 727 | UP | 143 | 138 | 265 |
| 728 | DOWN | 9234 | 10 | 3 |
| 729 | DOWN | 60 | 264 | 83 |
| 730 | UP | 5906 | 9 | 26 |
| 731 | UP | 1288 | 13 | 23 |
| 732 | UP | 915 | 1 | 9 |
| 733 | DOWN | 9891 | 9 | 2 |
| 734 | UP | 33 | 40 | 272 |
| 735 | DOWN | 11571 | 74 | 13 |
| 736 | UP | 1023 | 2 | 20 |
| 737 | DOWN | 670 | 61 | 59 |
| 738 | UP | 5187 | 9 | 29 |
| 739 | UP | 2096 | 80 | 87 |
| 740 | DOWN | 5360 | 62 | 17 |
| 741 | DOWN | 12680 | 36 | 10 |
| 742 | UP | 5239 | 3 | 29 |
| 743 | DOWN | 6926 | 11 | 4 |
| 744 | UP | 4793 | 73 | 144 |
| 745 | UP | 167 | 2 | 10 |
| 746 | UP | 7706 | 5 | 11 |
| 747 | DOWN | 4344 | 20 | 9 |
| 748 | UP | 2896 | 57 | 115 |
| 749 | UP | 2766 | 6 | 15 |
| 750 | UP | 1652 | 20 | 45 |
| 751 | UP | 1295 | 5 | 15 |
| 752 | UP | 1733 | 11 | 17 |
| 753 | UP | 1728 | 65 | 176 |
| 754 | UP | 1958 | 3 | 25 |
| 755 | UP | 1092 | 32 | 225 |
| 756 | UP | 5222 | 27 | 59 |
| 757 | DOWN | 8928 | 130 | 10 |
| 758 | UP | 7681 | 14 | 15 |
| 759 | DOWN | 3953 | 21 | 6 |
| 760 | DOWN | 9050 | 7 | 1 |
| 761 | DOWN | 3793 | 72 | 53 |
| 762 | DOWN | 10133 | 8 | 1 |
| 763 | UP | 584 | 5 | 8 |
| 764 | DOWN | 12494 | 9 | 2 |
| 765 | UP | 4129 | 58 | 78 |
| 766 | UP | 3727 | 9 | 20 |
| 767 | DOWN | 5730 | 9 | 2 |
| 768 | UP | 2340 | 45 | 198 |
| 769 | UP | 4532 | 16 | 22 |
| 770 | UP | 1198 | 94 | 277 |
| 771 | DOWN | 10387 | 39 | 7 |
| 772 | DOWN | 12955 | 15 | 7 |
| 773 | UP | 10 | 4481 | 8914 |
| 774 | DOWN | 2895 | 69 | 50 |
| 775 | UP | 3498 | 21 | 25 |
| 776 | UP | 8557 | 11 | 26 |
| 777 | UP | 6368 | 53 | 105 |
| 778 | UP | 2195 | 53 | 143 |
| 779 | UP | 716 | 84 | 203 |
| 780 | DOWN | 3192 | 42 | 10 |
| 781 | UP | 215 | 33 | 284 |
| 782 | UP | 3858 | 11 | 26 |
| 783 | UP | 9024 | 22 | 24 |
| 784 | UP | 497 | 5 | 38 |
| 785 | DOWN | 503 | 102 | 93 |
| 786 | UP | 449 | 56 | 83 |
| 787 | UP | 789 | 26 | 34 |
| 788 | DOWN | 11806 | 28 | 16 |
| 789 | DOWN | 1262 | 11 | 6 |
| 790 | UP | 4113 | 17 | 30 |
| 791 | UP | 1638 | 108 | 330 |
| 792 | UP | 985 | 18 | 38 |
| 793 | DOWN | 4070 | 21 | 8 |
| 794 | UP | 109 | 177 | 272 |
| 795 | UP | 1561 | 55 | 135 |
| 796 | UP | 3164 | 17 | 47 |
| 797 | UP | 31 | 448 | 557 |
| 798 | DOWN | 1397 | 20 | 14 |
| 799 | UP | 2 | 550 | 1174 |
| 800 | UP | 5322 | 11 | 78 |
| 801 | DOWN | 4813 | 36 | 6 |
| 802 | DOWN | 7099 | 105 | 12 |
| 803 | UP | 10758 | 6 | 32 |
| 804 | DOWN | 4950 | 22 | 7 |
| 805 | UP | 9211 | 9 | 11 |
| 806 | UP | 4718 | 6 | 30 |
| 807 | DOWN | 2581 | 11 | 2 |
| 808 | UP | 338 | 21 | 28 |
| 809 | UP | 2850 | 69 | 161 |
| 810 | UP | 5703 | 0 | 17 |
| 811 | UP | 1736 | 102 | 110 |
| 812 | DOWN | 9390 | 10 | 3 |
| 813 | UP | 7952 | 14 | 19 |
| 814 | DOWN | 3792 | 10 | 3 |
| 815 | DOWN | 9887 | 31 | 6 |
| 816 | DOWN | 27 | 12 | 4 |
| 817 | DOWN | 3451 | 9 | 0 |
| 818 | UP | 3047 | 2 | 34 |
| 819 | DOWN | 5186 | 145 | 24 |
| 820 | UP | 34 | 67 | 81 |
| 821 | UP | 1508 | 7 | 38 |
| 822 | DOWN | 11292 | 211 | 13 |
| 823 | UP | 2430 | 6 | 19 |
| 824 | UP | 2554 | 55 | 63 |
| 825 | DOWN | 10869 | 9 | 0 |
| 826 | UP | 9553 | 16 | 25 |
| 827 | DOWN | 8832 | 19 | 4 |
| 828 | DOWN | 5761 | 22 | 2 |
| 829 | DOWN | 2378 | 7 | 1 |
| 830 | DOWN | 8304 | 8 | 1 |
| 831 | UP | 4823 | 11 | 19 |
| 832 | DOWN | 4229 | 8 | 1 |
| 833 | UP | 2722 | 2 | 7 |
| 834 | UP | 13325 | 4 | 8 |
| 835 | UP | 1114 | 1 | 22 |
| 836 | UP | 6459 | 1 | 7 |
| 837 | UP | 2953 | 3 | 11 |
| 838 | UP | 807 | 31 | 73 |
| 839 | UP | 2040 | 1 | 9 |
| 840 | UP | 1336 | 58 | 290 |
| 841 | UP | 6796 | 6 | 15 |
| 842 | UP | 1455 | 7 | 22 |
| 843 | UP | 2576 | 29 | 145 |
| 844 | UP | 1293 | 19 | 117 |
| 845 | DOWN | 3518 | 18 | 1 |
| 846 | DOWN | 3819 | 10 | 9 |
| 847 | UP | 4734 | 22 | 38 |
| 848 | UP | 10265 | 0 | 9 |
| 849 | UP | 10344 | 4 | 23 |
| 850 | DOWN | 12491 | 18 | 11 |
| 851 | UP | 11621 | 2 | 18 |
| 852 | UP | 8384 | 7 | 20 |

### Example 4

### Pfam Annotation of Polypeptides of the Invention

Using the Pfam software program (Sonnhammer et al., Nucleic Acids Res., Vol. 26(1) pp. 320-322 (1998) herein incorporated by reference), the polypeptides of the invention (SEQ ID NO: 853 through 1704) we determined. Further description of the Pfam models can be found at http://pfam.wustl.edu/.

The following table lists the polypeptides of SEQ ID NO 853 through 1704 that had significant Pfam model hits:

TABLE 3

| SEQ ID | | | | | | |
|---|---|---|---|---|---|---|
| NO: | Model | Repeats | Position | Score | E_value | Description |
| 853 | TPR | 1/1 | 297-330 | 14.0 | 0.036 | TPR Domain |
| 854 | Sterol_desat | 1/1 | 24-214 | 268.2 | 2.9e−81 | Sterol |
| | | | | | | desaturase |
| 856 | ras | 1/1 | 8-194 | 335.5 | 1.4e−98 | Ras family |
| 857 | thyroglobulin_1 | 1/1 | 7-86 | 140.8 | 2.5e−38 | Thyroglobulin |
| | | | | | | type-1 repeat |
| 860 | thiored | 2/2 | 65-173 | 131.1 | 3.3e−38 | Thioredoxin |
| 861 | GP120 | 1/1 | 21-44 | 5.6 | 0.81 | Envelope |
| | | | | | | glycoprotein |
| | | | | | | GP120 |
| 861 | TIMP | 1/1 | 69-246 | 484.3 | 1.9e−176 | Tissue |
| | | | | | | inhibitor of |
| | | | | | | metalloproteinase |
| 863 | globin | 1/1 | 20-160 | 203.0 | 4.6e−57 | Globin |
| 864 | COX1 | 1/1 | 5-461 | 867.8 | 3.4e−257 | Cytochrome C |
| | | | | | | and Quinol |
| | | | | | | oxidase |
| | | | | | | polypeptide |
| 866 | PGAM | 1/1 | 13-241 | 436.5 | 2.3e−127 | Phosphoglycerate |
| | | | | | | mutase family |
| 869 | 2OG-FeII_Oxy | 1/1 | 117-146 | 15.5 | 0.005 | 2OG-Fe(II) |
| | | | | | | oxygenase |
| | | | | | | superfamily |
| 870 | LIM | 1/1 | 281-337 | 78.5 | 8.5e−21 | LIM domain |
| 870 | PDZ | 1/1 | 30-109 | 86.7 | 3e−23 | PDZ domain |
| | | | | | | (Also known as |
| | | | | | | DHR or GLGF) |
| 873 | GDPD | 1/1 | 243-324 | 73.6 | 4.1e−21 | Glycerophosphoryl |
| | | | | | | diester |
| | | | | | | phosphodiest |
| 874 | rrm | 1/3 | 204-274 | 48.1 | 1.1e−12 | RNA recognition |
| | | | | | | motif. (a.k.a. |
| | | | | | | RRM, RBD, |
| 874 | rrm | 2/3 | 301-372 | 112.3 | 1.3e−31 | RNA recognition |
| | | | | | | motif. (a.k.a. |
| | | | | | | RRM, RBD, |
| 874 | rrm | 3/3 | 496-552 | 20.9 | 0.00011 | RNA recognition |
| | | | | | | motif. (a.k.a. |
| | | | | | | RRM, RBD, |
| 875 | DUF232 | 1/1 | 396-433 | 9.7 | 0.36 | Putative |
| | | | | | | transcriptional |
| | | | | | | regulator |
| 875 | bZIP | 1/2 | 233-274 | 9.9 | 0.27 | bZIP |
| | | | | | | transcription |
| | | | | | | factor |
| 875 | bZIP | 2/2 | 408-430 | 9.2 | 0.44 | bZIP |
| | | | | | | transcription |
| | | | | | | factor |
| 875 | filament | 1/1 | 181-496 | 550.9 | 8.8e−162 | Intermediate |
| | | | | | | filament |
| | | | | | | protein |
| 878 | Zip | 1/1 | 209-354 | 168.7 | 9.9e−47 | ZIP Zinc |
| | | | | | | transporter |
| 879 | OATP_C | 1/1 | 78-445 | 669.4 | 1.9e−197 | Organic Anion |
| | | | | | | Transporter |
| | | | | | | Polypeptide |
| | | | | | | (OATP |
| 879 | OATP_N | 1/1 | 520-662 | 180.8 | 2.2e−50 | Organic Anion |
| | | | | | | Transporter |
| | | | | | | Polypeptide |
| | | | | | | (OATP |
| 879 | kazal | 1/1 | 471-498 | 14.8 | 0.051 | Kazal-type |
| | | | | | | serine protease |
| | | | | | | inhibitor |
| | | | | | | domain |
| 880 | G-gamma | 1/1 | 13-67 | 98.2 | 1.1e−30 | GGL domain |
| 881 | JNK | 1/1 | 55-311 | 495.0 | 1.1e−156 | Jun-like |
| | | | | | | transcription |
| | | | | | | factor |
| 881 | PspA_IM30 | 1/1 | 344-382 | 9.1 | 0.32 | PspA/IM30 |
| | | | | | | family |
| 881 | bZIP | 1/1 | 320-384 | 77.5 | 1.6e−20 | bZIP |
| | | | | | | transcription |
| | | | | | | factor |
| 882 | chloroa_b-bind | 1/1 | 69-93 | 28.3 | 5.6e−07 | Chlorophyll A-B |
| | | | | | | binding protein |
| 883 | chloroa_b-bind | 1/1 | 82-133 | 103.6 | 1.1e−28 | Chlorophyll A-B |
| | | | | | | binding protein |
| 884 | Glyco_transf_25 | 1/1 | 35-219 | 252.2 | 7.1e−72 | Glycosyltransferase |
| | | | | | | family 25 |
| | | | | | | (LPS bi |
| 885 | chloroa_b-bind | 1/1 | 78-120 | 84.7 | 3.1e−23 | Chlorophyll A-B |
| | | | | | | binding protein |
| 888 | DEP | 1/1 | 454-528 | 120.9 | 2.4e−32 | Domain found in |
| | | | | | | Dishevelled, |
| | | | | | | Egl-10, and |
| 888 | DIX | 1/1 | 33-114 | 196.2 | 5.1e−55 | DIX domain |
| 888 | Dishevelled | 1/1 | 174-245 | 138.7 | 1.7e−46 | Dishevelled |
| | | | | | | specific domain |
| 888 | PDZ | 1/1 | 281-368 | 87.8 | 1.5e−23 | PDZ domain |
| | | | | | | (Also known as |
| | | | | | | DHR or GLGF) |
| 889 | DUF232 | 1/2 | 282-316 | 8.5 | 0.72 | Putative |
| | | | | | | transcriptional |
| | | | | | | regulator |
| 889 | K-box | 1/1 | 176-193 | 7.8 | 0.52 | K-box region |
| 889 | filament | 1/1 | 158-474 | 527.7 | 8.2e−155 | Intermediate |
| | | | | | | filament |
| | | | | | | protein |
| 890 | ank | 2/3 | 84-116 | 11.9 | 0.14 | Ankyrin repeat |
| 890 | ank | 3/3 | 117-149 | 20.6 | 0.00046 | Ankyrin repeat |
| 891 | Herpes_HEPA | 1/1 | 85-99 | 3.4 | 0.86 | Herpesvirus DNA |
| | | | | | | helicase/primase |
| | | | | | | complex |
| 891 | actin | 1/1 | 77-279 | 440.8 | 9.4e−134 | Actin |
| 892 | ig | 1/4 | 41-100 | 36.7 | 1.6e−08 | Immunoglobulin |
| | | | | | | domain |
| 892 | ig | 2/4 | 175-236 | 30.1 | 1.1e−06 | Immunoglobulin |
| | | | | | | domain |
| 892 | ig | 3/4 | 274-333 | 19.2 | 0.0012 | Immunoglobulin |
| | | | | | | domain |
| 893 | Ribosomal_L14 | 1/1 | 32-153 | 218.5 | 9.7e−62 | Ribosomal |
| | | | | | | protein |
| | | | | | | L14p/L23e |
| 894 | PMP22_Claudin | 1/1 | 73-223 | 278.8 | 6.8e−80 | PMP- |
| | | | | | | 22/EMP/MP20/Claudin |
| | | | | | | family |
| 895 | alk_phosphatase | 1/1 | 41-512 | 1065.8 | 0 | Alkaline |
| | | | | | | phosphatase |
| 897 | CPSF_A | 1/1 | 821-1105 | 508.3 | 5.8e−149 | CPSF A subunit |
| | | | | | | region |
| 898 | Neur_chan_memb | 1/2 | 29-124 | 93.3 | 1.2e−26 | Neurotransmitter- |
| | | | | | | gated ion- |
| | | | | | | channel tra |
| 898 | Neur_chan_memb | 2/2 | 220-245 | 20.5 | 3.4e−05 | Neurotransmitter- |
| | | | | | | gated ion- |
| | | | | | | channel tra |
| 899 | G-patch | 1/1 | 2311-2355 | 70.7 | 3.9e−19 | G-patch domain |
| 899 | dsrm | 1/1 | 2378-2414 | 38.2 | 2.9e−09 | Double-stranded |
| | | | | | | RNA binding |
| | | | | | | motif |
| 900 | serpin | 1/1 | 9-423 | 515.3 | 1e−151 | Serpin (serine |
| | | | | | | protease |
| | | | | | | inhibitor) |
| 901 | Lipoprotein_X | 1/21 | 224-246 | 10.2 | 0.086 | Mycoplasma |
| | | | | | | MG185/MG260 |
| | | | | | | protein |
| 901 | Lipoprotein_X | 10/21 | 477-501 | 11.6 | 0.035 | Mycoplasma |
| | | | | | | MG185/MG260 |
| | | | | | | protein |
| 901 | Lipoprotein_X | 11/21 | 502-521 | 13.8 | 0.0086 | Mycoplasma |
| | | | | | | MG185/MG260 |
| | | | | | | protein |
| 901 | Lipoprotein_X | 12/21 | 523-541 | 12.8 | 0.017 | Mycoplasma |
| | | | | | | MG185/MG260 |
| | | | | | | protein |
| 901 | Lipoprotein_X | 13/21 | 543-566 | 18.6 | 0.00042 | Mycoplasma |
| | | | | | | MG185/MG260 |
| | | | | | | protein |
| 901 | Lipoprotein_X | 14/21 | 567-596 | 21.7 | 5.8e−05 | Mycoplasma |
| | | | | | | MG185/MG260 |
| | | | | | | protein |
| 901 | Lipoprotein_X | 15/21 | 597-621 | 13.7 | 0.0092 | Mycoplasma |
| | | | | | | MG185/MG260 |
| | | | | | | protein |
| 901 | Lipoprotein_X | 16/21 | 622-649 | 21.5 | 6.5e−05 | Mycoplasma |
| | | | | | | MG185/MG260 |
| | | | | | | protein |
| 901 | Lipoprotein_X | 17/21 | 652-671 | 12.5 | 0.021 | Mycoplasma |
| | | | | | | MG185/MG260 |
| | | | | | | protein |
| 901 | Lipoprotein_X | 18/21 | 672-696 | 15.6 | 0.0027 | Mycoplasma |
| | | | | | | MG185/MG260 |
| | | | | | | protein |
| 901 | Lipoprotein_X | 2/21 | 247-271 | 11.9 | 0.03 | Mycoplasma |
| | | | | | | MG185/MG260 |
| | | | | | | protein |
| 901 | Lipoprotein_X | 20/21 | 725-754 | 8.5 | 0.25 | Mycoplasma |
| | | | | | | MG185/MG260 |
| | | | | | | protein |
| 901 | Lipoprotein_X | 3/21 | 273-298 | 20.2 | 0.00015 | Mycoplasma |
| | | | | | | MG185/MG260 |
| | | | | | | protein |
| 901 | Lipoprotein_X | 4/21 | 303-329 | 17.8 | 0.0007 | Mycoplasma |
| | | | | | | MG185/MG260 |
| | | | | | | protein |
| 901 | Lipoprotein_X | 5/21 | 332-356 | 16.5 | 0.0016 | Mycoplasma |
| | | | | | | MG185/MG260 |
| | | | | | | protein |
| 901 | Lipoprotein_X | 6/21 | 357-386 | 19.5 | 0.00023 | Mycoplasma |
| | | | | | | MG185/MG260 |
| | | | | | | protein |
| 901 | Lipoprotein_X | 7/21 | 387-419 | 23.1 | 2.3e−05 | Mycoplasma |
| | | | | | | MG185/MG260 |
| | | | | | | protein |
| 901 | Lipoprotein_X | 8/21 | 422-446 | 17.8 | 0.0007 | Mycoplasma |
| | | | | | | MG185/MG260 |
| | | | | | | protein |
| 901 | Lipoprotein_X | 9/21 | 447-476 | 18.8 | 0.00035 | Mycoplasma |
| | | | | | | MG185/MG260 |
| | | | | | | protein |
| 902 | Seryl_tRNA_N | 1/1 | 27-78 | 76.9 | 4.2e−19 | Seryl-tRNA |
| | | | | | | synthetase N- |
| | | | | | | terminal domain |
| 902 | tRNA-synt_2b | 1/1 | 224-391 | 201.2 | 9e−59 | tRNA synthetase |
| | | | | | | class II core |
| | | | | | | domain (G, |
| 903 | CH | 1/1 | 81-196 | 41.3 | 5.2e−10 | Calponin |
| | | | | | | homology (CH) |
| | | | | | | domain |
| 903 | IQ | 1/3 | 730-750 | 23.4 | 0.00011 | IQ calmodulin- |
| | | | | | | binding motif |
| 903 | IQ | 2/3 | 760-780 | 22.6 | 0.00018 | IQ calmodulin- |
| | | | | | | binding motif |
| 903 | IQ | 3/3 | 790-810 | 13.7 | 0.063 | IQ calmodulin- |
| | | | | | | binding motif |
| 903 | Peptidase_C57 | 1/1 | 1233-1240 | 5.8 | 0.74 | Vaccinia virus |
| | | | | | | I7 processing |
| | | | | | | peptidase |
| 903 | RasGAP | 1/1 | 977-1190 | 341.7 | 8.1e−99 | GTPase- |
| | | | | | | activator |
| | | | | | | protein for |
| | | | | | | Ras-like G |
| 903 | RasGAP_C | 1/1 | 1405-1540 | 227.6 | 1.8e−64 | RasGAP C- |
| | | | | | | terminus |
| 903 | WW | 1/1 | 650-664 | 13.9 | 0.041 | WW domain |
| 904 | Alpha_L_fucos | 1/1 | 20-414 | 1057.3 | 0 | Alpha-L- |
| | | | | | | fucosidase |
| 906 | hormone | 1/1 | 19-106 | 61.2 | 2.3e−22 | Somatotropin |
| | | | | | | hormone family |
| 907 | rrm | 1/1 | 307-378 | 84.1 | 2.8e−23 | RNA recognition |
| | | | | | | motif. (a.k.a. |
| | | | | | | RRM, RBD, or |
| 911 | cyclin | 1/1 | 75-202 | 169.3 | 6.5e−47 | Cyclin, N- |
| | | | | | | terminal domain |
| 911 | cyclin_C | 1/1 | 204-332 | 78.7 | 6.1e−20 | Cyclin, C- |
| | | | | | | terminal domain |
| 912 | RIIa | 1/1 | 54-91 | 63.4 | 3.5e−16 | Regulatory |
| | | | | | | subunit of type |
| | | | | | | II PKA R-subu |
| 912 | cNMP_binding | 1/2 | 181-269 | 106.8 | 7.1e−29 | Cyclic |
| | | | | | | nucleotide- |
| | | | | | | binding domain |
| 912 | cNMP_binding | 2/2 | 299-393 | 117.7 | 5.9e−32 | Cyclic |
| | | | | | | nucleotide- |
| | | | | | | binding domain |
| 913 | DSBA | 1/1 | 61-76 | 12.5 | 0.023 | DSBA |
| | | | | | | oxidoreductase |
| 913 | RNA_pol_L | 1/1 | 35-127 | 200.3 | 4.6e−60 | RNA polymerases |
| | | | | | | L/13 to 16 kDa |
| | | | | | | subunit |
| 914 | Collagen | 1/21 | 602-650 | 37.1 | 1.1e−08 | Collagen triple |
| | | | | | | helix repeat |
| | | | | | | (20 copies) |
| 914 | Collagen | 10/21 | 941-971 | 19.6 | 0.00068 | Collagen triple |
| | | | | | | helix repeat |
| | | | | | | (20 copies) |
| 914 | Collagen | 11/21 | 984-1012 | 14.7 | 0.015 | Collagen triple |
| | | | | | | helix repeat |
| | | | | | | (20 copies) |
| 914 | Collagen | 17/21 | 1249-1269 | 8.0 | 1 | Collagen triple |
| | | | | | | helix repeat |
| | | | | | | (20 copies) |
| 914 | Collagen | 21/21 | 1473-1517 | 34.0 | 7.4e−08 | 20/21 1434 |
| | | | | | | 1446 . . . 113 |
| 914 | Collagen | 3/21 | 676-735 | 49.9 | 3.2e−12 | Collagen triple |
| | | | | | | helix repeat |
| | | | | | | (20 copies) |
| 914 | Collagen | 4/21 | 736-780 | 39.1 | 3e−09 | Collagen triple |
| | | | | | | helix repeat |
| | | | | | | (20 copies) |
| 914 | Collagen | 5/21 | 781-840 | 51.4 | 1.2e−12 | Collagen triple |
| | | | | | | helix repeat |
| | | | | | | (20 copies) |
| 914 | Collagen | 6/21 | 854-885 | 17.1 | 0.0033 | Collagen triple |
| | | | | | | helix repeat |
| | | | | | | (20 copies) |
| 914 | Seryl_tRNA_N | 1/1 | 520-537 | 12.5 | 0.13 | Seryl-tRNA |
| | | | | | | synthetase N- |
| | | | | | | terminal domain |
| 915 | GNT-I | 1/1 | 45-477 | 1084.9 | 0 | GNT-I family |
| 916 | CBM_14 | 2/6 | 922-942 | 9.2 | 0.14 | Chitin binding |
| | | | | | | Peritrophin-A |
| | | | | | | domain |
| 916 | CBM_14 | 4/6 | 1763-1783 | 10.7 | 0.047 | 3/6 1643 |
| | | | | | | 1665 . . . 3558 |
| 916 | CBM_14 | 5/6 | 1923-1943 | 11.4 | 0.028 | 3/6 1643 |
| | | | | | | 1665 . . . 3558 |
| 916 | EGF | 10/47 | 748-783 | 31.4 | 3.7e−07 | EGF-like domain |
| 916 | EGF | 11/47 | 858-894 | 41.8 | 4.1e−10 | EGF-like domain |
| 916 | EGF | 12/47 | 900-936 | 37.7 | 5.9e−09 | EGF-like domain |
| 916 | EGF | 13/47 | 942-976 | 27.1 | 6e−06 | EGF-like domain |
| 916 | EGF | 14/47 | 1045-1081 | 37.0 | 9.4e−09 | EGF-like domain |
| 916 | EGF | 15/47 | 1163-1199 | 31.6 | 3.1e−07 | EGF-like domain |
| 916 | EGF | 16/47 | 1205-1242 | 17.7 | 0.0026 | EGF-like domain |
| 916 | EGF | 17/47 | 1248-1284 | 28.1 | 3.1e−06 | EGF-like domain |
| 916 | EGF | 18/47 | 1290-1326 | 27.1 | 6e−06 | EGF-like domain |
| 916 | EGF | 19/47 | 1332-1367 | 22.5 | 0.00012 | EGF-like domain |
| 916 | EGF | 2/47 | 250-276 | 18.0 | 0.0021 | EGF-like domain |
| 916 | EGF | 20/47 | 1373-1409 | 31.9 | 2.5e−07 | EGF-like domain |
| 916 | EGF | 21/47 | 1415-1446 | 29.2 | 1.5e−06 | EGF-like domain |
| 916 | EGF | 22/47 | 1457-1492 | 26.5 | 8.7e−06 | EGF-like domain |
| 916 | EGF | 23/47 | 1498-1533 | 27.5 | 4.6e−06 | EGF-like domain |
| 916 | EGF | 24/47 | 1539-1575 | 33.4 | 1e−07 | EGF-like domain |
| 916 | EGF | 25/47 | 1581-1616 | 25.9 | 1.3e−05 | EGF-like domain |
| 916 | EGF | 26/47 | 1622-1657 | 25.6 | 1.6e−05 | EGF-like domain |
| 916 | EGF | 27/47 | 1741-1777 | 36.3 | 1.5e−08 | EGF-like domain |
| 916 | EGF | 28/47 | 1783-1818 | 15.9 | 0.0086 | EGF-like domain |
| 916 | EGF | 29/47 | 1901-1937 | 30.4 | 6.8e−07 | EGF-like domain |
| 916 | EGF | 3/47 | 281-308 | 16.9 | 0.0045 | EGF-like domain |
| 916 | EGF | 30/47 | 1943-1976 | 29.5 | 1.3e−06 | EGF-like domain |
| 916 | EGF | 31/47 | 1984-2020 | 21.0 | 0.00031 | EGF-like domain |
| 916 | EGF | 32/47 | 2026-2059 | 17.6 | 0.0029 | EGF-like domain |
| 916 | EGF | 33/47 | 2065-2102 | 37.8 | 5.5e−09 | EGF-like domain |
| 916 | EGF | 34/47 | 2108-2142 | 25.1 | 2.2e−05 | EGF-like domain |
| 916 | EGF | 35/47 | 2148-2184 | 32.8 | 1.4e−07 | EGF-like domain |
| 916 | EGF | 36/47 | 2262-2295 | 22.9 | 9e−05 | EGF-like domain |
| 916 | EGF | 37/47 | 2301-2335 | 23.8 | 5.1e−05 | EGF-like domain |
| 916 | EGF | 38/47 | 2341-2376 | 26.0 | 1.2e−05 | EGF-like domain |
| 916 | EGF | 39/47 | 2382-2420 | 20.0 | 0.00061 | EGF-like domain |
| 916 | EGF | 4/47 | 381-417 | 20.2 | 0.00051 | EGF-like domain |
| 916 | EGF | 40/47 | 2426-2462 | 25.7 | 1.5e−05 | EGF-like domain |
| 916 | EGF | 41/47 | 2537-2573 | 30.4 | 6.9e−07 | EGF-like domain |
| 916 | EGF | 42/47 | 2579-2614 | 33.5 | 9e−08 | EGF-like domain |
| 916 | EGF | 43/47 | 2620-2650 | 18.0 | 0.0022 | EGF-like domain |
| 916 | EGF | 44/47 | 2659-2696 | 32.4 | 1.9e−07 | EGF-like domain |
| 916 | EGF | 45/47 | 2702-2736 | 16.6 | 0.0054 | EGF-like domain |
| 916 | EGF | 46/47 | 2742-2777 | 20.6 | 0.00041 | EGF-like domain |
| 916 | EGF | 47/47 | 2783-2817 | 13.8 | 0.034 | EGF-like domain |
| 916 | EGF | 5/47 | 423-459 | 30.1 | 8.6e−07 | EGF-like domain |
| 916 | EGF | 6/47 | 584-614 | 24.5 | 3.2e−05 | EGF-like domain |
| 916 | EGF | 7/47 | 625-659 | 23.3 | 6.8e−05 | EGF-like domain |
| 916 | EGF | 8/47 | 665-701 | 38.3 | 4e−09 | EGF-like domain |
| 916 | EGF | 9/47 | 707-742 | 42.0 | 3.7e−10 | EGF-like domain |
| 916 | TB | 1/10 | 324-366 | 72.5 | 2.4e−21 | TB domain |
| 916 | TB | 10/10 | 2477-2520 | 76.0 | 1.9e−22 | TB domain |
| 916 | TB | 2/10 | 474-519 | 69.6 | 2e−20 | TB domain |
| 916 | TB | 3/10 | 799-841 | 75.9 | 2e−22 | TB domain |
| 916 | TB | 4/10 | 991-1032 | 59.3 | 3.6e−17 | TB domain |
| 916 | TB | 6/10 | 1096-1138 | 71.2 | 6.2e−21 | TB domain |
| 916 | TB | 7/10 | 1678-1719 | 79.2 | 1.8e−23 | TB domain |
| 916 | TB | 8/10 | 1835-1878 | 79.5 | 1.5e−23 | TB domain |
| 916 | TB | 9/10 | 2199-2241 | 73.0 | 1.7e−21 | TB domain |
| 916 | wnt | 1/1 | 434-445 | 3.4 | 0.78 | wnt family |
| 917 | Fragilysin | 1/1 | 448-495 | 6.8 | 0.39 | Fragilysin |
| | | | | | | metallopeptidase |
| | | | | | | (M10C) en |
| 917 | Peptidase_M10 | 1/1 | 160-266 | 276.2 | 4.1e−79 | Matrixin |
| 917 | Peptidase_M10_N | 1/1 | 80-154 | 141.4 | 1.3e−48 | Matrix |
| | | | | | | metalloprotease, |
| | | | | | | N-terminal do |
| 917 | UPF0143 | 1/1 | 540-560 | 6.2 | 0.89 | Uncharacterised |
| | | | | | | protein family |
| | | | | | | (UPF01 |
| 917 | fn2 | 1/3 | 281-322 | 92.4 | 3.9e−34 | Fibronectin |
| | | | | | | type II domain |
| 917 | fn2 | 2/3 | 339-380 | 91.9 | 6.1e−34 | Fibronectin |
| | | | | | | type II domain |
| 917 | fn2 | 3/3 | 397-438 | 104.0 | 1.4e−38 | Fibronectin |
| | | | | | | type II domain |
| 917 | hemopexin | 1/4 | 523-566 | 42.4 | 2.7e−11 | Hemopexin |
| 917 | hemopexin | 2/4 | 568-611 | 54.2 | 6.8e−15 | Hemopexin |
| 917 | hemopexin | 3/4 | 616-663 | 73.0 | 1.3e−20 | Hemopexin |
| 917 | hemopexin | 4/4 | 665-708 | 31.9 | 4.4e−08 | Hemopexin |
| 918 | CD36 | 1/1 | 28-468 | 797.9 | 3.8e−236 | CD36 family |
| 920 | TSC22 | 1/1 | 141-200 | 146.4 | 4.8e−46 | TSC-22/dip/bun |
| | | | | | | family |
| 921 | AMOP | 1/1 | 483-646 | 319.0 | 7e−96 | AMOP domain |
| 921 | tsp_1 | 1/1 | 417-457 | 25.2 | 4.8e−06 | Thrombospondin |
| | | | | | | type 1 domain |
| 923 | Peptidase_C1 | 1/1 | 85-334 | 393.4 | 3.7e−117 | Papain family |
| | | | | | | cysteine |
| | | | | | | protease |
| 926 | secY | 1/3 | 87-108 | 11.6 | 0.013 | eubacterial |
| | | | | | | secY protein |
| 926 | secY | 3/3 | 278-472 | 28.2 | 1.7e−07 | eubacterial |
| | | | | | | secY protein |
| 927 | DUF173 | 1/1 | 583-629 | 16.4 | 0.0014 | Uncharacterized |
| | | | | | | ACR, COG1354 |
| 928 | arf | 1/1 | 24-202 | 423.3 | 2.1e−123 | ADP- |
| | | | | | | ribosylation |
| | | | | | | factor family |
| 930 | EF1BD | 1/1 | 175-261 | 198.7 | 1.5e−56 | EF-1 guanine |
| | | | | | | nucleotide |
| | | | | | | exchange domain |
| 931 | Myc-LZ | 1/1 | 360-390 | 8.5 | 0.75 | Myc leucine |
| | | | | | | zipper domain |
| 931 | bZIP | 1/1 | 327-391 | 21.5 | 0.00014 | bZIP |
| | | | | | | transcription |
| | | | | | | factor |
| 933 | CAP | 1/1 | 13-483 | 1121.9 | 0 | CAP protein |
| 934 | 7tm_1 | 1/1 | 179-431 | 255.5 | 4.1e−82 | 7 transmembrane |
| | | | | | | receptor |
| | | | | | | (rhodopsin |
| | | | | | | family) |
| 935 | NadA | 1/1 | 18-38 | 7.9 | 0.78 | Quinolinate |
| | | | | | | synthetase A |
| | | | | | | protein |
| 935 | RrnaAD | 1/1 | 133-169 | 5.2 | 0.78 | Ribosomal RNA |
| | | | | | | adenine |
| | | | | | | dimethylase |
| 935 | efhand | 1/4 | 34-62 | 39.6 | 4.8e−09 | EF hand |
| 935 | efhand | 2/4 | 70-98 | 38.4 | 1e−08 | EF hand |
| 935 | efhand | 3/4 | 107-135 | 42.1 | 9.8e−10 | EF hand |
| 935 | efhand | 4/4 | 143-171 | 41.2 | 1.7e−09 | EF hand |
| 936 | rrm | 1/2 | 107-176 | 59.4 | 5e−16 | RNA recognition |
| | | | | | | motif. (a.k.a. |
| | | | | | | RRM, RBD, or |
| 936 | rrm | 2/2 | 194-237 | 43.0 | 3.4e−11 | RNA recognition |
| | | | | | | motif. (a.k.a. |
| | | | | | | RRM, RBD, or |
| 939 | Vps55 | 1/1 | 26-154 | 54.2 | 2.2e−14 | Vacuolar |
| | | | | | | protein sorting |
| | | | | | | 55 |
| 940 | SH2 | 1/1 | 110-191 | 53.8 | 2.3e−17 | SH2 domain |
| 943 | CH | 1/1 | 57-169 | 79.5 | 1.4e−20 | Calponin |
| | | | | | | homology (CH) |
| | | | | | | domain |
| 943 | calponin | 1/1 | 206-231 | 51.5 | 2e−14 | Calponin family |
| | | | | | | repeat |
| 946 | PSI_PsaJ | 1/1 | 185-220 | 7.8 | 0.76 | Photosystem I |
| | | | | | | reaction centre |
| | | | | | | subunit IX/P |
| 947 | YT521-B | 1/1 | 436-526 | 210.7 | 2.2e−59 | YT521-B-like |
| | | | | | | family |
| 950 | HSP20 | 1/1 | 80-176 | 73.5 | 4.9e−19 | Hsp20/alpha |
| | | | | | | crystallin |
| | | | | | | family |
| 951 | CH | 1/2 | 55-158 | 99.5 | 4.2e−26 | Calponin |
| | | | | | | homology (CH) |
| | | | | | | domain |
| 951 | CH | 2/2 | 168-273 | 127.8 | 2e−34 | Calponin |
| | | | | | | homology (CH) |
| | | | | | | domain |
| 951 | efhand | 1/2 | 777-805 | 22.5 | 0.00025 | EF hand |
| 951 | efhand | 2/2 | 818-846 | 20.1 | 0.0012 | EF hand |
| 951 | spectrin | 1/4 | 299-409 | 62.5 | 7.6e−16 | Spectrin repeat |
| 951 | spectrin | 2/4 | 419-524 | 108.6 | 1.2e−28 | Spectrin repeat |
| 951 | spectrin | 3/4 | 535-646 | 56.3 | 4.1e−14 | Spectrin repeat |
| 951 | spectrin | 4/4 | 657-760 | 56.3 | 4.2e−14 | Spectrin repeat |
| 952 | abhydrolase | 1/1 | 172-407 | 50.2 | 2.1e−14 | alpha/beta |
| | | | | | | hydrolase fold |
| 953 | NIF | 1/1 | 164-338 | 358.8 | 1.4e−104 | NLI interacting |
| | | | | | | factor |
| 954 | PMP22_Claudin | 1/1 | 12-127 | 149.3 | 5.1e−42 | PMP- |
| | | | | | | 22/EMP/MP20/Claudin |
| | | | | | | family |
| 955 | Exo_endo_phos | 1/1 | 400-699 | 140.8 | 2.4e−38 | Endonuclease/Ex |
| | | | | | | onuclease/phosphatase |
| | | | | | | fa |
| 956 | rrm | 1/1 | 49-115 | 78.6 | 1.1e−21 | RNA recognition |
| | | | | | | motif. (a.k.a. |
| | | | | | | RRM, RBD, or |
| 956 | zf-CCHC | 1/1 | 140-157 | 20.4 | 0.00095 | Zinc knuckle |
| 958 | SH3 | 1/1 | 365-413 | 52.4 | 9.2e−13 | SH3 domain |
| 959 | ELH | 1/1 | 247-273 | 4.4 | 0.71 | Egg-laying |
| | | | | | | hormone |
| | | | | | | precursor |
| 959 | TGF-beta | 1/1 | 337-445 | 218.2 | 1.3e−62 | Transforming |
| | | | | | | growth factor |
| | | | | | | beta like |
| 959 | TGFb_propeptide | 1/1 | 61-293 | 194.0 | 2.3e−54 | TGF-beta |
| | | | | | | propeptide |
| 959 | Tub | 1/1 | 255-264 | 2.3 | 0.33 | Tub family |
| 960 | Hydrolase | 2/3 | 347-379 | 13.2 | 0.026 | haloacid |
| | | | | | | dehalogenase- |
| | | | | | | like hydrolase |
| 961 | PABP | 1/1 | 544-615 | 166.7 | 3.8e−46 | Poly-adenylate |
| | | | | | | binding |
| | | | | | | protein, unique |
| 961 | rrm | 1/4 | 14-85 | 98.4 | 1.7e−27 | RNA recognition |
| | | | | | | motif. (a.k.a. |
| | | | | | | RRM, RBD |
| 961 | rrm | 2/4 | 102-171 | 90.8 | 2.8e−25 | RNA recognition |
| | | | | | | motif. (a.k.a. |
| | | | | | | RRM, RBD |
| 961 | rrm | 3/4 | 194-264 | 110.9 | 3.6e−31 | RNA recognition |
| | | | | | | motif. (a.k.a. |
| | | | | | | RRM, RBD |
| 961 | rrm | 4/4 | 297-366 | 86.5 | 5.3e−24 | RNA recognition |
| | | | | | | motif. (a.k.a. |
| | | | | | | RRM, RBD |
| 962 | bZIP | 1/1 | 672-735 | 17.2 | 0.0023 | bZIP |
| | | | | | | transcription |
| | | | | | | factor |
| 964 | COLFI | 1/1 | 948-1165 | 561.6 | 5.7e−219 | Fibrillar |
| | | | | | | collagen C- |
| | | | | | | terminal domain |
| 964 | Collagen | 1/13 | 105-142 | 20.6 | 0.00035 | Collagen triple |
| | | | | | | helix repeat |
| | | | | | | (20 copies |
| 964 | Collagen | 10/13 | 657-716 | 44.5 | 9.7e−11 | Collagen triple |
| | | | | | | helix repeat |
| | | | | | | (20 copies |
| 964 | Collagen | 11/13 | 717-776 | 56.8 | 4e−14 | Collagen triple |
| | | | | | | helix repeat |
| | | | | | | (20 copies |
| 964 | Collagen | 12/13 | 780-839 | 56.3 | 5.6e−14 | Collagen triple |
| | | | | | | helix repeat |
| | | | | | | (20 copies |
| 964 | Collagen | 13/13 | 840-896 | 58.7 | 1.2e−14 | Collagen triple |
| | | | | | | helix repeat |
| | | | | | | (20 copies |
| 964 | Collagen | 2/13 | 171-230 | 64.5 | 3e−16 | Collagen triple |
| | | | | | | helix repeat |
| | | | | | | (20 copies |
| 964 | Collagen | 3/13 | 237-296 | 76.5 | 1.6e−19 | Collagen triple |
| | | | | | | helix repeat |
| | | | | | | (20 copies |
| 964 | Collagen | 4/13 | 297-356 | 66.3 | 9.5e−17 | Collagen triple |
| | | | | | | helix repeat |
| | | | | | | (20 copies |
| 964 | Collagen | 5/13 | 357-416 | 65.2 | 1.9e−16 | Collagen triple |
| | | | | | | helix repeat |
| | | | | | | (20 copies |
| 964 | Collagen | 6/13 | 417-476 | 65.6 | 1.5e−16 | Collagen triple |
| | | | | | | helix repeat |
| | | | | | | (20 copies |
| 964 | Collagen | 7/13 | 477-536 | 61.9 | 1.6e−15 | Collagen triple |
| | | | | | | helix repeat |
| | | | | | | (20 copies |
| 964 | Collagen | 8/13 | 537-596 | 67.3 | 5.2e−17 | Collagen triple |
| | | | | | | helix repeat |
| | | | | | | (20 copies |
| 964 | Collagen | 9/13 | 597-656 | 47.1 | 1.8e−11 | Collagen triple |
| | | | | | | helix repeat |
| | | | | | | (20 copies |
| 964 | DNA_po13_beta | 1/1 | 925-947 | 9.3 | 0.39 | DNA polymerase |
| | | | | | | III beta |
| | | | | | | subunit, N-term |
| 965 | DEAD | 1/1 | 205-430 | 273.3 | 4.3e−83 | DEAD/DEAH box |
| | | | | | | helicase |
| 965 | helicase_C | 1/1 | 477-548 | 130.3 | 1.4e−35 | Helicase |
| | | | | | | conserved c- |
| | | | | | | terminal domain |
| 966 | zf-RanBP | 1/1 | 85-114 | 32.7 | 2.8e−07 | Zn-finger in |
| | | | | | | Ran binding |
| | | | | | | protein and |
| | | | | | | others |
| 967 | DUF343 | 1/1 | 12-131 | 158.7 | 3.2e−44 | Protein of |
| | | | | | | unknown |
| | | | | | | function |
| | | | | | | (DUF343) |
| 969 | UPF0005 | 1/1 | 281-347 | 19.4 | 0.00018 | Uncharacterized |
| | | | | | | protein family |
| | | | | | | UPF0005 |
| 970 | GSHPx | 1/1 | 45-159 | 245.6 | 7e−70 | Glutathione |
| | | | | | | peroxidase |
| 970 | RTX | 1/1 | 48-57 | 5.2 | 0.22 | RTX N-terminal |
| | | | | | | domain |
| 971 | Insulin | 1/1 | 122-179 | 84.7 | 1.8e−21 | Insulin/IGF/Rel |
| | | | | | | axin family |
| 972 | 2OG-FeII_Oxy | 1/1 | 353-451 | 52.8 | 1.7e−13 | 2OG-Fe(II) |
| | | | | | | oxygenase |
| | | | | | | superfamily |
| 975 | ank | 2/7 | 61-93 | 33.7 | 8.2e−08 | Ankyrin repeat |
| 975 | ank | 3/7 | 94-126 | 22.1 | 0.00017 | Ankyrin repeat |
| 975 | ank | 4/7 | 127-159 | 29.4 | 1.4e−06 | Ankyrin repeat |
| 975 | ank | 5/7 | 160-192 | 44.2 | 8.8e−11 | Ankyrin repeat |
| 975 | ank | 6/7 | 193-225 | 38.0 | 4.9e−09 | Ankyrin repeat |
| 979 | Glyco_transf_29 | 1/1 | 86-373 | 419.0 | 4.3e−122 | Glycosyltransferase |
| | | | | | | family 29 |
| | | | | | | (sialyl |
| 980 | TIM | 1/1 | 82-254 | 383.9 | 3.8e−124 | Triosephosphate |
| | | | | | | isomerase |
| 981 | adh_short | 1/1 | 60-308 | 297.7 | 1.4e−85 | short chain |
| | | | | | | dehydrogenase |
| 982 | Ribosomal_L24e | 1/1 | 40-110 | 177.5 | 2.5e−51 | Ribosomal |
| | | | | | | protein L24e |
| 985 | annexin | 1/4 | 63-130 | 116.8 | 4.1e−31 | Annexin |
| 985 | annexin | 2/4 | 135-202 | 109.0 | 9e−29 | Annexin |
| 985 | annexin | 3/4 | 219-287 | 88.8 | 1.1e−22 | Annexin |
| 985 | annexin | 4/4 | 295-362 | 106.8 | 4.2e−28 | Annexin |
| 989 | Fz | 1/1 | 147-266 | 213.2 | 6.3e−72 | Fz domain |
| 989 | NTR | 1/1 | 298-402 | 80.9 | 1.2e−20 | NTR/C345C |
| | | | | | | module |
| 990 | WW | 1/1 | 151-180 | 48.0 | 1e−11 | WW domain |
| 991 | Glyco_hydro_35 | 1/1 | 6-66 | 89.4 | 4.7e−26 | Glycosyl |
| | | | | | | hydrolases |
| | | | | | | family 35 |
| 993 | PKI | 1/1 | 15-84 | 98.0 | 1.2e−27 | cAMP-dependent |
| | | | | | | protein kinase |
| | | | | | | inhibitor |
| 994 | RWP-RK | 1/1 | 63-74 | 8.4 | 0.71 | RWP-RK domain |
| 994 | Stathmin | 1/1 | 26-165 | 311.6 | 9.2e−90 | Stathmin family |
| 999 | Peptidase_M1 | 1/1 | 235-619 | 659.8 | 6.6e−228 | Peptidase |
| | | | | | | family M1 |
| 1000 | Peptidase_C1 | 1/1 | 256-483 | 323.3 | 3.8e−96 | Papain family |
| | | | | | | cysteine |
| | | | | | | protease |
| 1001 | IF-2B | 1/1 | 15-298 | 436.2 | 3e−127 | Initiation |
| | | | | | | factor 2 |
| | | | | | | subunit family |
| 1002 | Cu_amine_oxid | 1/2 | 315-722 | 751.6 | 3.3e−222 | Copper amine |
| | | | | | | oxidase, enzyme |
| | | | | | | domain |
| 1002 | Cu_amine_oxid | 2/2 | 723-759 | 61.9 | 1.3e−16 | Copper amine |
| | | | | | | oxidase, enzyme |
| | | | | | | domain |
| 1002 | Cu_amine_oxidN2 | 1/1 | 58-145 | 115.9 | 2.5e−32 | Copper amine |
| | | | | | | oxidase, N2 |
| | | | | | | domain |
| 1002 | Cu_amine_oxidN3 | 1/1 | 161-261 | 99.6 | 1.4e−28 | Copper amine |
| | | | | | | oxidase, N3 |
| | | | | | | domain |
| 1003 | DUF279 | 1/1 | 98-110 | 6.8 | 1 | Eukaryotic |
| | | | | | | protein of |
| | | | | | | unknown |
| | | | | | | function |
| 1003 | TRAPP_Bet3 | 1/1 | 58-238 | 418.5 | 6.1e−122 | Transport |
| | | | | | | protein |
| | | | | | | particle |
| | | | | | | (TRAPP) com |
| 1004 | zf-CCCH | 1/2 | 114-140 | 49.6 | 2.8e−13 | Zinc finger C- |
| | | | | | | x8-C-x5-C-x3-H |
| | | | | | | type (and simil |
| 1004 | zf-CCCH | 2/2 | 152-178 | 48.1 | 8e−13 | Zinc finger C- |
| | | | | | | x8-C-x5-C-x3-H |
| | | | | | | type (and simil |
| 1007 | Peptidase_M13 | 1/1 | 544-750 | 262.3 | 4.3e−81 | Peptidase |
| | | | | | | family M13 |
| 1007 | Peptidase_M4 | 1/1 | 582-594 | 11.6 | 0.079 | Thermolysin |
| | | | | | | metallopeptidase, |
| | | | | | | catalytic |
| 1008 | Cadherin_C_term | 1/1 | 661-817 | 280.3 | 2.5e−80 | Cadherin |
| | | | | | | cytoplasmic |
| | | | | | | region |
| 1008 | cadherin | 1/5 | 92-182 | 62.9 | 5.1e−17 | Cadherin domain |
| 1008 | cadherin | 2/5 | 196-289 | 109.3 | 1.3e−30 | Cadherin domain |
| 1008 | cadherin | 3/5 | 303-404 | 122.1 | 2.2e−34 | Cadherin domain |
| 1008 | cadherin | 4/5 | 417-510 | 94.1 | 3.7e−26 | Cadherin domain |
| 1008 | cadherin | 5/5 | 523-616 | 42.1 | 6.5e−11 | Cadherin domain |
| 1010 | KH | 1/3 | 32-78 | 57.3 | 5.9e−15 | KH domain |
| 1010 | KH | 2/3 | 116-165 | 63.1 | 1.2e−16 | KH domain |
| 1010 | KH | 3/3 | 298-346 | 58.3 | 3e−15 | KH domain |
| 1011 | Glyco_hydro_2_N | 1/1 | 78-101 | 19.8 | 0.00014 | Glycosyl |
| | | | | | | hydrolases |
| | | | | | | family 2, sugar b |
| 1012 | C2 | 1/3 | 158-241 | 86.9 | 1.3e−23 | C2 domain |
| 1012 | C2 | 3/3 | 724-757 | 21.5 | 9.4e−05 | C2 domain |
| 1013 | PA28_alpha | 1/1 | 32-95 | 155.4 | 1e−42 | Proteasome |
| | | | | | | activator pa28 |
| | | | | | | alpha subunit |
| 1013 | PA28_beta | 1/1 | 126-275 | 409.9 | 2.4e−119 | Proteasome |
| | | | | | | activator pa28 |
| | | | | | | beta subunit |
| 1013 | biopterin_H | 1/1 | 59-69 | 6.8 | 0.18 | Biopterin- |
| | | | | | | dependent |
| | | | | | | aromatic amino |
| | | | | | | acid h |
| 1014 | ras | 1/1 | 27-129 | 80.3 | 8.8e−23 | Ras family |
| 1015 | Rhabdo_glycop | 1/1 | 392-439 | 10.6 | 0.019 | Rhabdovirus |
| | | | | | | spike |
| | | | | | | glycoprotein |
| 1015 | ig | 1/3 | 90-174 | 22.9 | 0.00011 | Immunoglobulin |
| | | | | | | domain |
| 1015 | ig | 3/3 | 308-362 | 23.1 | 9.7e−05 | Immunoglobulin |
| | | | | | | domain |
| 1016 | EGF | 1/5 | 264-300 | 15.1 | 0.014 | EGF-like domain |
| 1016 | EGF | 2/5 | 306-343 | 35.3 | 2.9e−08 | EGF-like domain |
| 1016 | EGF | 3/5 | 349-380 | 20.9 | 0.00033 | EGF-like domain |
| 1016 | EGF | 4/5 | 389-425 | 19.1 | 0.0011 | EGF-like domain |
| 1016 | EGF | 5/5 | 431-467 | 15.5 | 0.011 | EGF-like domain |
| 1016 | TIL | 1/2 | 297-349 | 9.9 | 0.094 | Trypsin |
| | | | | | | Inhibitor like |
| | | | | | | cysteine rich |
| | | | | | | do |
| 1016 | Tissue_fac | 1/1 | 583-625 | 20.1 | 0.00014 | Tissue_factor |
| 1016 | Xlink | 1/1 | 33-64 | 7.8 | 0.008 | Extracellular |
| | | | | | | link domain |
| 1016 | lectin_c | 1/1 | 47-103 | 20.3 | 0.00082 | Lectin C-type |
| | | | | | | domain |
| 1018 | FH2 | 1/1 | 1-59 | 57.2 | 9.5e−15 | Formin Homology |
| | | | | | | 2 Domain |
| 1019 | GoLoco | 1/1 | 64-84 | 33.6 | 1.4e−08 | LGN motif, |
| | | | | | | putative GEF |
| | | | | | | specific for G- |
| | | | | | | alpha |
| 1019 | Rap_GAP | 1/1 | 277-464 | 424.3 | 1.1e−123 | Rap/ran-GAP |
| 1020 | Cys_knot | 1/1 | 297-394 | 114.6 | 1.8e−30 | Cystine−knot |
| | | | | | | domain |
| 1020 | IGFBP | 1/1 | 77-148 | 115.5 | 1.4e−31 | Insulin-like |
| | | | | | | growth factor |
| | | | | | | binding protein |
| 1020 | tsp_1 | 1/1 | 249-290 | 56.2 | 2.7e−15 | Thrombospondin |
| | | | | | | type 1 domain |
| 1020 | vwc | 2/2 | 151-214 | 102.8 | 1.9e−28 | von Willebrand |
| | | | | | | factor type C |
| | | | | | | domain |
| 1021 | rrm | 1/2 | 9-72 | 77.8 | 1.9e−21 | RNA recognition |
| | | | | | | motif. (a.k.a. |
| | | | | | | RRM, RBD, or |
| 1021 | rrm | 2/2 | 208-274 | 49.7 | 3.5e−13 | RNA recognition |
| | | | | | | motif. (a.k.a. |
| | | | | | | RRM, RBD, or |
| 1022 | Ets | 1/1 | 409-492 | 206.7 | 3.5e−58 | Ets-domain |
| 1023 | CRF | 1/1 | 192-230 | 70.0 | 9.6e−19 | Corticotropin- |
| | | | | | | releasing |
| | | | | | | factor family |
| 1024 | Exonuclease | 1/1 | 64-236 | 103.6 | 1.5e−27 | Exonuclease |
| 1026 | OPR | 1/1 | 59-88 | 50.1 | 9.8e−13 | Octicosapeptide |
| | | | | | | repeat |
| 1027 | SSF | 1/2 | 112-279 | 28.8 | 1.7e−07 | Sodium: solute |
| | | | | | | symporter |
| | | | | | | family |
| 1027 | SSF | 2/2 | 400-514 | 12.4 | 0.0086 | Sodium: solute |
| | | | | | | symporter |
| | | | | | | family |
| 1029 | Ribosomal_S9 | 1/1 | 19-151 | 294.9 | 3.2e−91 | Ribosomal |
| | | | | | | protein S9/S16 |
| 1031 | actin | 1/1 | 24-399 | 942.3 | 2.3e−286 | Actin |
| 1032 | gln-synt | 1/1 | 249-502 | 364.6 | 1.1e−105 | Glutamine |
| | | | | | | synthetase, |
| | | | | | | catalytic |
| | | | | | | domain |
| 1032 | gln-synt_N | 1/1 | 163-244 | 128.6 | 1.6e−37 | Glutamine |
| | | | | | | synthetase, |
| | | | | | | beta-Grasp |
| | | | | | | domain |
| 1034 | sushi | 1/3 | 71-117 | 12.5 | 0.15 | Sushi domain |
| | | | | | | (SCR repeat) |
| 1034 | sushi | 2/3 | 137-192 | 54.2 | 1.5e−12 | Sushi domain |
| | | | | | | (SCR repeat) |
| 1034 | sushi | 3/3 | 199-252 | 54.9 | 9.6e−13 | Sushi domain |
| | | | | | | (SCR repeat) |
| 1034 | trypsin | 1/1 | 515-786 | 155.7 | 1.2e−48 | Trypsin |
| 1034 | vwa | 1/1 | 304-502 | 221.2 | 7.1e−64 | von Willebrand |
| | | | | | | factor type A |
| | | | | | | domain |
| 1036 | efhand | 1/6 | 90-118 | 19.0 | 0.0023 | EF hand |
| 1036 | efhand | 2/6 | 126-154 | 9.6 | 0.91 | EF hand |
| 1036 | efhand | 4/6 | 210-238 | 21.3 | 0.00053 | EF hand |
| 1036 | efhand | 6/6 | 287-315 | 9.8 | 0.81 | EF hand |
| 1037 | bZIP | 1/1 | 75-135 | 78.1 | 1.1e−20 | bZIP |
| | | | | | | transcription |
| | | | | | | factor |
| 1038 | AhpC-TSA | 1/1 | 16-165 | 310.5 | 2e−89 | AhpC/TSA family |
| 1039 | IBR | 1/1 | 340-389 | 7.3 | 0.81 | IBR domain |
| 1039 | zf-C2H2 | 1/5 | 319-344 | 13.5 | 0.26 | Zinc finger, |
| | | | | | | C2H2 type |
| 1039 | zf-C2H2 | 2/5 | 350-374 | 22.3 | 0.0018 | Zinc finger, |
| | | | | | | C2H2 type |
| 1039 | zf-C2H2 | 3/5 | 380-402 | 26.1 | 0.00019 | Zinc finger, |
| | | | | | | C2H2 type |
| 1039 | zf-C2H2 | 4/5 | 408-430 | 25.7 | 0.00024 | Zinc finger, |
| | | | | | | C2H2 type |
| 1039 | zf-C2H2 | 5/5 | 438-461 | 23.5 | 0.00085 | Zinc finger, |
| | | | | | | C2H2 type |
| 1040 | EGF | 2/2 | 664-691 | 13.2 | 0.052 | EGF-like domain |
| 1040 | FAD_binding_7 | 1/1 | 388-412 | 6.0 | 1 | FAD binding |
| | | | | | | domain of DNA |
| | | | | | | photolyase |
| 1040 | Pep_M12B_propep | 1/1 | 86-201 | 164.7 | 7.6e−46 | Reprolysin |
| | | | | | | family |
| | | | | | | propeptide |
| 1040 | Reprolysin | 1/1 | 218-420 | 400.8 | 1.3e−116 | Reprolysin |
| | | | | | | (M12B) family |
| | | | | | | zinc metallo |
| 1040 | disintegrin | 1/1 | 437-512 | 102.0 | 8.2e−33 | Disintegrin |
| 1041 | Na_H_antiporter | 1/1 | 477-523 | 8.8 | 0.26 | Na+/H+ |
| | | | | | | antiporter |
| | | | | | | family |
| 1041 | Na_sulph_symp | 1/2 | 19-174 | 124.1 | 1.3e−34 | Sodium: sulfate |
| | | | | | | symporter |
| | | | | | | transmembran |
| 1041 | Na_sulph_symp | 2/2 | 225-585 | 200.5 | 2.5e−56 | Sodium: sulfate |
| | | | | | | symporter |
| | | | | | | transmembran |
| 1042 | MDM2 | 1/1 | 177-189 | 11.1 | 0.017 | p53-associated |
| | | | | | | protein (MDM2) |
| 1042 | NAD_binding_1 | 1/1 | 65-160 | 48.5 | 1.5e−12 | Oxidoreductase |
| | | | | | | NAD-binding |
| | | | | | | domain |
| 1043 | trypsin | 1/1 | 95-331 | 330.0 | 4e−104 | Trypsin |
| 1045 | EGF | 1/18 | 83-110 | 27.0 | 6.3e−06 | EGF-like domain |
| 1045 | EGF | 10/18 | 803-838 | 26.0 | 1.2e−05 | EGF-like domain |
| 1045 | EGF | 11/18 | 844-879 | 21.2 | 0.00027 | EGF-like domain |
| 1045 | EGF | 12/18 | 885-921 | 24.5 | 3.3e−05 | EGF-like domain |
| 1045 | EGF | 13/18 | 927-963 | 24.7 | 2.9e−05 | EGF-like domain |
| 1045 | EGF | 14/18 | 969-1006 | 22.1 | 0.00015 | EGF-like domain |
| 1045 | EGF | 15/18 | 1107-1144 | 20.2 | 0.00053 | EGF-like domain |
| 1045 | EGF | 16/18 | 1150-1185 | 30.4 | 6.7e−07 | EGF-like domain |
| 1045 | EGF | 17/18 | 1304-1339 | 30.8 | 5.2e−07 | EGF-like domain |
| 1045 | EGF | 18/18 | 1345-1384 | 31.5 | 3.5e−07 | EGF-like domain |
| 1045 | EGF | 2/18 | 310-345 | 30.6 | 6.1e−07 | EGF-like domain |
| 1045 | EGF | 4/18 | 556-592 | 29.8 | 1e−06 | EGF-like domain |
| 1045 | EGF | 5/18 | 598-634 | 28.1 | 3.2e−06 | EGF-like domain |
| 1045 | EGF | 6/18 | 640-661 | 9.2 | 0.68 | EGF-like domain |
| 1045 | EGF | 8/18 | 721-756 | 24.6 | 3e−05 | EGF-like domain |
| 1045 | EGF | 9/18 | 762-797 | 26.4 | 9.3e−06 | EGF-like domain |
| 1045 | Fanconia | 1/1 | 9-31 | 5.6 | 0.94 | Fanconi anaemia |
| | | | | | | group A protein |
| 1045 | TB | 1/4 | 246-289 | 54.5 | 1.1e−15 | TB domain |
| 1045 | TB | 2/4 | 367-407 | 70.6 | 9.5e−21 | TB domain |
| 1045 | TB | 3/4 | 1036-1079 | 74.2 | 7.2e−22 | TB domain |
| 1045 | TB | 4/4 | 1213-1255 | 76.3 | 1.5e−22 | TB domain |
| 1045 | VSP | 1/3 | 660-681 | 7.4 | 0.3 | |
| 1045 | VSP | 3/3 | 907-927 | 6.9 | 0.41 | |
| 1045 | granulin | 1/2 | 621-636 | 7.4 | 0.58 | Granulin |
| 1048 | CSD | 1/8 | 53-118 | 77.9 | 1.8e−20 | ‘Cold-shock’ |
| | | | | | | DNA-binding |
| | | | | | | domain |
| 1048 | CSD | 3/8 | 213-276 | 89.0 | 1.3e−23 | 2/8 180 |
| | | | | | | 207 . . . 4471 |
| 1048 | CSD | 4/8 | 337-365 | 9.7 | 0.31 | 2/8 180 |
| | | | | | | 207 . . . 4471 |
| 1048 | CSD | 5/8 | 376-424 | 28.5 | 1.6e−06 | 2/8 180 |
| | | | | | | 207 . . . 4471 |
| 1048 | CSD | 6/8 | 514-538 | 11.3 | 0.11 | 2/8 180 |
| | | | | | | 207 . . . 4471 |
| 1048 | CSD | 7/8 | 546-610 | 76.5 | 4.6e−20 | 2/8 180 |
| | | | | | | 207 . . . 4471 |
| 1048 | CSD | 8/8 | 701-766 | 74.9 | 1.2e−19 | 2/8 180 |
| | | | | | | 207 . . . 4471 |
| 1050 | DIL | 1/1 | 451-557 | 160.9 | 2.2e−44 | DIL domain |
| 1050 | FHA | 1/1 | 92-158 | 50.0 | 1.5e−11 | FHA domain |
| 1050 | PDZ | 1/1 | 675-758 | 86.3 | 4e−23 | PDZ domain |
| | | | | | | (Also known as |
| | | | | | | DHR or GLGF) |
| 1051 | Lamp | 1/1 | 65-438 | 832.2 | 1.7e−246 | Lysosome- |
| | | | | | | associated |
| | | | | | | membrane |
| | | | | | | glycoprot |
| 1052 | pp-binding | 1/1 | 103-170 | 69.7 | 1.1e−17 | Phosphopantetheine |
| | | | | | | attachment |
| | | | | | | site |
| 1053 | START | 1/1 | 174-324 | 58.6 | 4.1e−16 | START domain |
| 1055 | HSP70 | 1/1 | 620-643 | 4.7 | 0.87 | Hsp70 protein |
| 1055 | helicase_C | 1/1 | 643-732 | 50.3 | 8.6e−13 | Helicase |
| | | | | | | conserved C- |
| | | | | | | terminal domain |
| 1056 | Calpain_III | 1/1 | 385-542 | 387.0 | 2.6e−113 | Calpain large |
| | | | | | | subunit, domain |
| | | | | | | III |
| 1056 | Peptidase_C2 | 1/1 | 75-374 | 739.1 | 1.9e−218 | Calpain family |
| | | | | | | cysteine |
| | | | | | | protease |
| 1056 | Porin_2 | 1/1 | 683-706 | 5.9 | 0.69 | Porin subfamily |
| 1056 | efhand | 2/4 | 609-637 | 14.7 | 0.036 | EF hand |
| 1056 | efhand | 3/4 | 639-667 | 16.6 | 0.01 | EF hand |
| 1057 | KOW | 1/1 | 135-169 | 34.8 | 1.3e−07 | KOW motif |
| 1058 | TGF-beta | 1/1 | 260-367 | 121.4 | 2.7e−34 | Transforming |
| | | | | | | growth factor |
| | | | | | | beta like |
| | | | | | | domain |
| 1062 | ig | 1/2 | 65-142 | 13.7 | 0.039 | Immunoglobulin |
| | | | | | | domain |
| 1062 | ig | 2/2 | 172-235 | 21.2 | 0.00032 | Immunoglobulin |
| | | | | | | domain |
| 1063 | EGF | 2/2 | 436-451 | 12.1 | 0.1 | EGF-like domain |
| 1063 | Glyco_hydro_56 | 1/1 | 38-376 | 785.1 | 2.7e−232 | Hyaluronidase |
| 1064 | DUF232 | 1/2 | 278-329 | 14.6 | 0.017 | Putative |
| | | | | | | transcriptional |
| | | | | | | regulator |
| 1064 | KE2 | 1/1 | 315-347 | 9.6 | 0.39 | KE2 family |
| | | | | | | protein |
| 1064 | filament | 1/1 | 97-409 | 568.1 | 5.6e−167 | Intermediate |
| | | | | | | filament |
| | | | | | | protein |
| 1067 | PKD | 1/2 | 280-319 | 25.1 | 9.2e−07 | PKD domain |
| 1067 | PKD | 2/2 | 448-476 | 7.0 | 0.5 | PKD domain |
| 1068 | zf-B_box | 1/1 | 228-268 | 55.4 | 1.2e−12 | B-box zinc |
| | | | | | | finger |
| 1069 | ras | 1/1 | 25-216 | 372.2 | 1.9e−109 | Ras family |
| 1070 | IRS | 1/1 | 234-336 | 206.5 | 3.9e−58 | PTB domain |
| | | | | | | (IRS-1 type) |
| 1070 | PH | 1/1 | 71-184 | 47.1 | 7.1e−12 | PH domain |
| 1071 | Peptidase_M16 | 1/1 | 98-246 | 240.8 | 2e−68 | Insulinase |
| | | | | | | (Peptidase |
| | | | | | | family M16) |
| 1072 | CBM_20 | 1/1 | 279-356 | 31.5 | 4e−07 | Starch binding |
| | | | | | | domain |
| 1073 | PRA1 | 1/1 | 80-240 | 280.6 | 2e−80 | Prenylated rab |
| | | | | | | acceptor (PRA1) |
| 1075 | CLN3 | 1/1 | 48-449 | 889.8 | 8.1e−264 | CLN3 protein |
| 1075 | FA_desaturase | 1/1 | 52-83 | 11.7 | 0.019 | Fatty acid |
| | | | | | | desaturase |
| 1076 | UPF0146 | 1/1 | 2989-3007 | 7.2 | 0.81 | Uncharacterised |
| | | | | | | protein family |
| | | | | | | (UPF0146 |
| 1076 | laminin_B | 2/2 | 1715-1851 | 83.2 | 7.3e−22 | Laminin B |
| | | | | | | (Domain IV) |
| 1076 | laminin_EGF | 1/22 | 322-378 | 30.1 | 4.1e−07 | Laminin EGF- |
| | | | | | | like (Domains |
| | | | | | | III and V) |
| 1076 | laminin_EGF | 10/22 | 798-848 | 43.0 | 8.4e−11 | Laminin EGF- |
| | | | | | | like (Domains |
| | | | | | | III and V) |
| 1076 | laminin_EGF | 11/22 | 851-883 | 29.0 | 8.8e−07 | Laminin EGF- |
| | | | | | | like (Domains |
| | | | | | | III and V) |
| 1076 | laminin_EGF | 12/22 | 1460-1508 | 58.1 | 4e−15 | Laminin EGF- |
| | | | | | | like (Domains |
| | | | | | | III and V) |
| 1076 | laminin_EGF | 14/22 | 1550-1596 | 44.6 | 2.9e−11 | Laminin EGF- |
| | | | | | | like (Domains |
| | | | | | | III and V) |
| 1076 | laminin_EGF | 15/22 | 1599-1647 | 65.4 | 3.1e−17 | Laminin EGF- |
| | | | | | | like (Domains |
| | | | | | | III and V) |
| 1076 | laminin_EGF | 16/22 | 1853-1883 | 15.1 | 0.0083 | Laminin EGF- |
| | | | | | | like (Domains |
| | | | | | | III and V) |
| 1076 | laminin_EGF | 17/22 | 1886-1932 | 51.9 | 2.2e−13 | Laminin EGF- |
| | | | | | | like (Domains |
| | | | | | | III and V) |
| 1076 | laminin_EGF | 18/22 | 1935-1988 | 51.4 | 3.2e−13 | Laminin EGF- |
| | | | | | | like (Domains |
| | | | | | | III and V) |
| 1076 | laminin_EGF | 19/22 | 1991-2042 | 65.3 | 3.3e−17 | Laminin EGF- |
| | | | | | | like (Domains |
| | | | | | | III and V) |
| 1076 | laminin_EGF | 2/22 | 381-448 | 45.4 | 1.6e−11 | Laminin EGF- |
| | | | | | | like (Domains |
| | | | | | | III and V) |
| 1076 | laminin_EGF | 20/22 | 2045-2089 | 45.4 | 1.7e−11 | Laminin EGF- |
| | | | | | | like (Domains |
| | | | | | | III and V) |
| 1076 | laminin_EGF | 21/22 | 2092-2136 | 47.8 | 3.5e−12 | Laminin EGF- |
| | | | | | | like (Domains |
| | | | | | | III and V) |
| 1076 | laminin_EGF | 22/22 | 2139-2167 | 16.0 | 0.0048 | Laminin EGF- |
| | | | | | | like (Domains |
| | | | | | | III and V) |
| 1076 | laminin_EGF | 3/22 | 451-493 | 34.1 | 3e−08 | Laminin EGF- |
| | | | | | | like (Domains |
| | | | | | | III and V) |
| 1076 | laminin_EGF | 4/22 | 516-560 | 47.1 | 5.7e−12 | Laminin EGF- |
| | | | | | | like (Domains |
| | | | | | | III and V) |
| 1076 | laminin_EGF | 5/22 | 563-606 | 40.4 | 4.5e−10 | Laminin EGF- |
| | | | | | | like (Domains |
| | | | | | | III and V) |
| 1076 | laminin_EGF | 6/22 | 609-651 | 51.4 | 3.3e−13 | Laminin EGF- |
| | | | | | | like (Domains |
| | | | | | | III and V) |
| 1076 | laminin_EGF | 7/22 | 654-696 | 40.6 | 4e−10 | Laminin EGF- |
| | | | | | | like (Domains |
| | | | | | | III and V) |
| 1076 | laminin_EGF | 8/22 | 699-747 | 45.0 | 2.2e−11 | Laminin EGF- |
| | | | | | | like (Domains |
| | | | | | | III and V) |
| 1076 | laminin_EGF | 9/22 | 766-795 | 38.7 | 1.4e−09 | Laminin EGF- |
| | | | | | | like (Domains |
| | | | | | | III and V) |
| 1076 | laminin_G | 1/4 | 2898-2933 | 9.0 | 0.27 | Laminin G |
| | | | | | | domain |
| 1076 | laminin_G | 3/4 | 3392-3524 | 45.2 | 1.3e−11 | Laminin G |
| | | | | | | domain |
| 1076 | laminin_G | 4/4 | 3571-3698 | 60.6 | 5.7e−16 | Laminin G |
| | | | | | | domain |
| 1076 | laminin_Nterm | 1/1 | 67-320 | 285.9 | 3.8e−86 | Laminin N- |
| | | | | | | terminal |
| | | | | | | (Domain VI) |
| 1077 | KOW | 1/1 | 12-47 | 31.2 | 1.2e−06 | KOW motif |
| 1077 | Ribosomal_L27e | 1/1 | 59-143 | 213.6 | 7.4e−74 | Ribosomal L27e |
| | | | | | | protein family |
| 1078 | arf | 1/1 | 64-240 | 144.3 | 1e−39 | ADP- |
| | | | | | | ribosylation |
| | | | | | | factor family |
| 1080 | UPF0005 | 1/1 | 99-279 | 137.6 | 9.1e−39 | Uncharacterized |
| | | | | | | protein family |
| | | | | | | UPF0005 |
| 1081 | cyclin | 1/1 | 81-208 | 163.2 | 4.4e−45 | Cyclin, N- |
| | | | | | | terminal domain |
| 1081 | cyclin_C | 1/1 | 210-339 | 73.0 | 2.1e−18 | Cyclin, C- |
| | | | | | | terminal domain |
| 1082 | Oxysterol_BP | 1/1 | 442-858 | 772.2 | 2.1e−228 | Oxysterol- |
| | | | | | | binding protein |
| 1082 | PH | 1/1 | 150-242 | 87.5 | 2.9e−23 | PH domain |
| 1083 | DUF348 | 1/1 | 110-129 | 11.6 | 0.14 | Domain of |
| | | | | | | unknown |
| | | | | | | function |
| | | | | | | (DUF348) |
| 1083 | rnaseH | 1/1 | 600-733 | 28.5 | 1e−06 | RNase H |
| 1083 | rve | 1/1 | 863-963 | 27.5 | 9.7e−07 | Integrase core |
| | | | | | | domain |
| 1083 | rvp | 1/1 | 63-84 | 25.9 | 7.8e−06 | Retroviral |
| | | | | | | aspartyl |
| | | | | | | protease |
| 1083 | rvt | 1/1 | 185-357 | 46.1 | 1.4e−11 | Reverse |
| | | | | | | transcriptase |
| | | | | | | (RNA-dependent |
| | | | | | | DNA pol |
| 1084 | MAP1_LC3 | 1/1 | 40-143 | 255.6 | 6.1e−75 | Microtubule |
| | | | | | | associated |
| | | | | | | protein 1A/1B, |
| | | | | | | light |
| 1085 | 7tm_1 | 1/2 | 50-88 | 8.2 | 0.045 | 7 transmembrane |
| | | | | | | receptor |
| | | | | | | (rhodopsin |
| | | | | | | family) |
| 1089 | SlyX | 1/1 | 273-283 | 6.3 | 0.019 | SlyX |
| 1090 | aa_permeases | 2/2 | 359-410 | 12.5 | 0.012 | |
| 1092 | adh_short | 1/1 | 28-228 | 149.0 | 4.1e−41 | short chain |
| | | | | | | dehydrogenase |
| 1094 | Clusterin | 1/1 | 16-463 | 1269.5 | 0 | Clusterin |
| 1094 | Thymidylate_kin | 1/1 | 338-380 | 9.1 | 0.024 | Thymidylate |
| | | | | | | kinase |
| 1096 | COLFI | 1/1 | 1248-1466 | 568.8 | 9.2e−222 | Fibrillar |
| | | | | | | collagen C- |
| | | | | | | terminal domain |
| 1096 | Collagen | 1/18 | 112-161 | 27.3 | 5e−06 | Collagen triple |
| | | | | | | helix repeat |
| | | | | | | (20 copies) |
| 1096 | Collagen | 10/18 | 659-718 | 60.6 | 3.6e−15 | Collagen triple |
| | | | | | | helix repeat |
| | | | | | | (20 copies) |
| 1096 | Collagen | 11/18 | 719-778 | 70.2 | 8.4e−18 | Collagen triple |
| | | | | | | helix repeat |
| | | | | | | (20 copies) |
| 1096 | Collagen | 12/18 | 782-841 | 68.4 | 2.7e−17 | Collagen triple |
| | | | | | | helix repeat |
| | | | | | | (20 copies) |
| 1096 | Collagen | 13/18 | 842-901 | 60.4 | 4e−15 | Collagen triple |
| | | | | | | helix repeat |
| | | | | | | (20 copies) |
| 1096 | Collagen | 14/18 | 902-961 | 59.2 | 8.9e−15 | Collagen triple |
| | | | | | | helix repeat |
| | | | | | | (20 copies) |
| 1096 | Collagen | 15/18 | 962-1021 | 62.6 | 9.9e−16 | Collagen triple |
| | | | | | | helix repeat |
| | | | | | | (20 copies) |
| 1096 | Collagen | 16/18 | 1023-1081 | 54.4 | 1.8e−13 | Collagen triple |
| | | | | | | helix repeat |
| | | | | | | (20 copies) |
| 1096 | Collagen | 17/18 | 1082-1141 | 73.9 | 8.1e−19 | Collagen triple |
| | | | | | | helix repeat |
| | | | | | | (20 copies) |
| 1096 | Collagen | 18/18 | 1142-1195 | 40.5 | 1.2e−09 | Collagen triple |
| | | | | | | helix repeat |
| | | | | | | (20 copies) |
| 1096 | Collagen | 2/18 | 180-238 | 50.4 | 2.3e−12 | Collagen triple |
| | | | | | | helix repeat |
| | | | | | | (20 copies) |
| 1096 | Collagen | 3/18 | 239-298 | 75.7 | 2.5e−19 | Collagen triple |
| | | | | | | helix repeat |
| | | | | | | (20 copies) |
| 1096 | Collagen | 4/18 | 299-358 | 64.9 | 2.4e−16 | Collagen triple |
| | | | | | | helix repeat |
| | | | | | | (20 copies) |
| 1096 | Collagen | 5/18 | 359-418 | 61.6 | 1.9e−15 | Collagen triple |
| | | | | | | helix repeat |
| | | | | | | (20 copies) |
| 1096 | Collagen | 6/18 | 419-478 | 61.1 | 2.6e−15 | Collagen triple |
| | | | | | | helix repeat |
| | | | | | | (20 copies) |
| 1096 | Collagen | 7/18 | 479-538 | 63.9 | 4.4e−16 | Collagen triple |
| | | | | | | helix repeat |
| | | | | | | (20 copies) |
| 1096 | Collagen | 8/18 | 539-598 | 64.6 | 2.9e−16 | Collagen triple |
| | | | | | | helix repeat |
| | | | | | | (20 copies) |
| 1096 | Collagen | 9/18 | 599-658 | 62.1 | 1.4e−15 | Collagen triple |
| | | | | | | helix repeat |
| | | | | | | (20 copies) |
| 1098 | DUF410 | 1/1 | 66-315 | 513.5 | 1.6e−150 | Protein of |
| | | | | | | unknown |
| | | | | | | function |
| | | | | | | (DUF410) |
| 1098 | v110 | 1/1 | 306-326 | 8.7 | 0.55 | Viral family |
| | | | | | | 110 |
| 1100 | G-alpha | 1/1 | 59-407 | 820.0 | 8.4e−243 | G-protein alpha |
| | | | | | | subunit |
| 1100 | arf | 1/1 | 244-279 | 25.6 | 4e−06 | ADP- |
| | | | | | | ribosylation |
| | | | | | | factor family |
| 1101 | ECH | 1/1 | 367-539 | 73.0 | 1.4e−20 | Enoyl-coA |
| | | | | | | hydratase/isomerase |
| | | | | | | family |
| 1101 | chromo | 1/1 | 77-113 | 53.3 | 3.5e−14 | ‘chromo’ |
| | | | | | | (CHRromatin |
| | | | | | | Organization |
| | | | | | | MOdi |
| 1102 | MAM | 1/1 | 27-184 | 212.9 | 4.7e−60 | MAM domain |
| 1102 | Y_phosphatase | 1/2 | 923-1153 | 432.5 | 3.9e−126 | Protein- |
| | | | | | | tyrosine |
| | | | | | | phosphatase |
| 1102 | Y_phosphatase | 2/2 | 1213-1447 | 326.5 | 3.1e−94 | Protein- |
| | | | | | | tyrosine |
| | | | | | | phosphatase |
| 1102 | bromodomain | 1/1 | 1008-1024 | 7.8 | 0.79 | Bromodomain |
| 1102 | fn3 | 1/4 | 281-367 | 32.1 | 8e−08 | Fibronectin |
| | | | | | | type III domain |
| 1102 | fn3 | 2/4 | 379-470 | 8.2 | 0.67 | Fibronectin |
| | | | | | | type III domain |
| 1102 | fn3 | 3/4 | 482-575 | 42.5 | 8e−11 | Fibronectin |
| | | | | | | type III domain |
| 1102 | ig | 1/1 | 199-262 | 23.1 | 9.9e−05 | Immunoglobulin |
| | | | | | | domain |
| 1104 | PWWP | 1/1 | 114-186 | 123.0 | 4.9e−34 | PWWP domain |
| 1105 | Bcl-2 | 1/1 | 233-332 | 165.6 | 2.1e−66 | Apoptosis |
| | | | | | | regulator |
| | | | | | | proteins, Bcl-2 |
| | | | | | | family |
| 1106 | sugar_tr | 1/1 | 15-468 | 661.4 | 4.6e−195 | Sugar (and |
| | | | | | | other) |
| | | | | | | transporter |
| 1107 | C2 | 1/1 | 521-600 | 52.4 | 1.1e−13 | C2 domain |
| 1107 | Calpain_III | 1/1 | 353-498 | 269.1 | 1.9e−78 | Calpain large |
| | | | | | | subunit, domain |
| | | | | | | III |
| 1107 | Peptidase_C2 | 1/1 | 29-347 | 283.1 | 3.5e−81 | Calpain family |
| | | | | | | cysteine |
| | | | | | | protease |
| 1108 | DSPc | 1/1 | 317-457 | 244.7 | 1.3e−69 | Dual |
| | | | | | | specificity |
| | | | | | | phosphatase, |
| | | | | | | catalytic |
| 1108 | Rhodanese | 1/2 | 130-167 | 27.7 | 5.9e−07 | Rhodanese-like |
| | | | | | | domain |
| 1108 | Y_phosphatase | 1/1 | 387-436 | 6.3 | 0.81 | Protein- |
| | | | | | | tyrosine |
| | | | | | | phosphatase |
| 1109 | UPF0128 | 1/1 | 467-482 | 6.5 | 0.99 | Uncharacterised |
| | | | | | | protein family |
| | | | | | | (UPF0128) |
| 1109 | WH2 | 1/1 | 1160-1180 | 14.6 | 0.023 | WH2 motif |
| 1110 | rrm | 1/2 | 420-474 | 13.8 | 0.013 | RNA recognition |
| | | | | | | motif. (a.k.a. |
| | | | | | | RRM, RBD, or |
| 1110 | rrm | 2/2 | 504-572 | 26.8 | 2e−06 | RNA recognition |
| | | | | | | motif. (a.k.a. |
| | | | | | | RRM, RBD, or |
| 1112 | Fork_head | 1/1 | 55-191 | 93.0 | 6.1e−24 | Fork head |
| | | | | | | domain |
| 1113 | thyroglobulin_1 | 1/1 | 156-228 | 104.2 | 2.6e−27 | Thyroglobulin |
| | | | | | | type-1 repeat |
| 1114 | E1_DerP2_DerF2 | 1/1 | 51-178 | 185.6 | 8e−52 | ML domain |
| 1115 | ENV_polyprotein | 1/1 | 2-28 | 17.1 | 0.00015 | ENV polyprotein |
| | | | | | | (coat |
| | | | | | | polyprotein) |
| 1116 | heme_1 | 1/1 | 104-184 | 53.8 | 6.5e−14 | Heme/Steroid |
| | | | | | | binding domain |
| 1117 | IGFBP | 1/1 | 87-145 | 99.4 | 5.7e−27 | Insulin-like |
| | | | | | | growth factor |
| | | | | | | binding pr |
| 1117 | thyroglobulin_1 | 1/1 | 234-309 | 142.1 | 1e−38 | Thyroglobulin |
| | | | | | | type-1 repeat |
| 1119 | EGF | 2/6 | 181-216 | 26.3 | 9.7e−06 | EGF-like domain |
| 1119 | EGF | 3/6 | 222-256 | 23.6 | 5.9e−05 | EGF-like domain |
| 1119 | EGF | 4/6 | 262-296 | 20.1 | 0.00056 | EGF-like domain |
| 1119 | EGF | 5/6 | 302-329 | 12.5 | 0.078 | EGF-like domain |
| 1119 | TIL | 1/4 | 203-222 | 10.7 | 0.051 | Trypsin |
| | | | | | | Inhibitor like |
| | | | | | | cysteine rich |
| | | | | | | domain |
| 1119 | TIL | 2/4 | 241-262 | 15.7 | 0.0014 | Trypsin |
| | | | | | | Inhibitor like |
| | | | | | | cysteine rich |
| | | | | | | domain |
| 1119 | VSP | 2/2 | 282-304 | 10.4 | 0.045 | 1/2 242 |
| | | | | | | 266..129 |
| 1120 | ZU5 | 1/1 | 989-1093 | 218.3 | 1.1e−61 | ZU5 domain |
| 1120 | ank | 10/24 | 340-372 | 32.1 | 2.4e−07 | Ankyrin repeat |
| 1120 | ank | 11/24 | 373-405 | 35.8 | 2.2e−08 | Ankyrin repeat |
| 1120 | ank | 12/24 | 406-438 | 45.1 | 4.7e−11 | Ankyrin repeat |
| 1120 | ank | 13/24 | 439-471 | 37.2 | 8.7e−09 | Ankyrin repeat |
| 1120 | ank | 14/24 | 472-504 | 37.4 | 7.3e−09 | Ankyrin repeat |
| 1120 | ank | 15/24 | 505-537 | 38.3 | 4.1e−09 | Ankyrin repeat |
| 1120 | ank | 16/24 | 538-570 | 42.1 | 3.4e−10 | Ankyrin repeat |
| 1120 | ank | 17/24 | 571-603 | 40.2 | 1.2e−09 | Ankyrin repeat |
| 1120 | ank | 18/24 | 604-636 | 35.9 | 2e−08 | Ankyrin repeat |
| 1120 | ank | 19/24 | 637-669 | 44.4 | 7.4e−11 | Ankyrin repeat |
| 1120 | ank | 2/24 | 80-112 | 40.3 | 1.1e−09 | Ankyrin repeat |
| 1120 | ank | 20/24 | 670-702 | 29.0 | 1.9e−06 | Ankyrin repeat |
| 1120 | ank | 21/24 | 703-735 | 41.2 | 6.2e−10 | Ankyrin repeat |
| 1120 | ank | 22/24 | 736-768 | 38.5 | 3.7e−09 | Ankyrin repeat |
| 1120 | ank | 23/24 | 769-801 | 39.1 | 2.5e−09 | Ankyrin repeat |
| 1120 | ank | 3/24 | 113-145 | 42.6 | 2.4e−10 | Ankyrin repeat |
| 1120 | ank | 4/24 | 146-178 | 39.2 | 2.2e−09 | Ankyrin repeat |
| 1120 | ank | 5/24 | 179-202 | 20.6 | 0.00046 | Ankyrin repeat |
| 1120 | ank | 6/24 | 212-240 | 10.0 | 0.46 | Ankyrin repeat |
| 1120 | ank | 7/24 | 241-273 | 32.9 | 1.4e−07 | Ankyrin repeat |
| 1120 | ank | 8/24 | 274-306 | 44.7 | 6.2e−11 | Ankyrin repeat |
| 1120 | ank | 9/24 | 307-339 | 34.1 | 6.6e−08 | Ankyrin repeat |
| 1120 | death | 1/1 | 4098-4181 | 109.4 | 1e−30 | Death domain |
| 1121 | LIM | 1/2 | 30-87 | 77.0 | 2.2e−20 | LIM domain |
| 1121 | LIM | 2/2 | 90-147 | 40.0 | 6.9e−10 | LIM domain |
| 1121 | PDZ | 1/1 | 170-256 | 70.0 | 1.8e−18 | PDZ domain |
| | | | | | | (Also known as |
| | | | | | | DHR or GLGF) |
| 1121 | pkinase | 1/1 | 349-626 | 213.8 | 2.5e−60 | Protein kinase |
| | | | | | | domain |
| 1122 | Yippee | 1/1 | 23-131 | 263.8 | 2.2e−75 | Yippee putative |
| | | | | | | zinc-binding |
| | | | | | | protein |
| 1123 | Kunitz_BPTI | 1/1 | 29-79 | 94.2 | 1.6e−29 | Kunitz/Bovine |
| | | | | | | pancreatic |
| | | | | | | trypsin |
| | | | | | | inhibito |
| 1125 | NHL | 1/1 | 73-100 | 33.7 | 8e−08 | NHL repeat |
| 1126 | PUF | 1/8 | 861-895 | 33.8 | 4.8e−08 | Pumilio-family |
| | | | | | | RNA binding |
| | | | | | | repeat |
| 1126 | PUF | 2/8 | 897-931 | 32.3 | 1.4e−07 | Pumilio-family |
| | | | | | | RNA binding |
| | | | | | | repeat |
| 1126 | PUF | 3/8 | 933-967 | 33.2 | 7.5e−08 | Pumilio-family |
| | | | | | | RNA binding |
| | | | | | | repeat |
| 1126 | PUF | 4/8 | 969-1003 | 40.3 | 6.7e−10 | Pumilio-family |
| | | | | | | RNA binding |
| | | | | | | repeat |
| 1126 | PUF | 5/8 | 1005-1039 | 37.1 | 5.4e−09 | Pumilio-family |
| | | | | | | RNA binding |
| | | | | | | repeat |
| 1126 | PUF | 6/8 | 1041-1075 | 45.2 | 2.6e−11 | Pumilio-family |
| | | | | | | RNA binding |
| | | | | | | repeat |
| 1126 | PUF | 7/8 | 1077-1112 | 28.1 | 2.2e−06 | Pumilio-family |
| | | | | | | RNA binding |
| | | | | | | repeat |
| 1126 | PUF | 8/8 | 1120-1154 | 31.0 | 3.3e−07 | Pumilio-family |
| | | | | | | RNA binding |
| | | | | | | repeat |
| 1127 | Ribosomal_L7Ae | 1/1 | 30-112 | 35.4 | 3.5e−09 | Ribosomal |
| | | | | | | protein |
| | | | | | | L7Ae/L30e/S12e/ |
| | | | | | | Gadd4 |
| 1128 | S10_plectin | 1/1 | 21-118 | 254.1 | 1.9e−72 | Plectin/S10 |
| | | | | | | domain |
| 1133 | CBM_14 | 2/2 | 1273-1302 | 11.4 | 0.028 | Chitin binding |
| | | | | | | Peritrophin-A |
| | | | | | | domain |
| 1133 | Cys_knot | 2/2 | 2823-2919 | 100.4 | 3.4e−26 | Cystine-knot |
| | | | | | | domain |
| 1133 | EB | 2/3 | 914-927 | 11.8 | 0.19 | |
| 1133 | TIL | 1/5 | 401-454 | 41.0 | 1.5e−11 | Trypsin |
| | | | | | | Inhibitor like |
| | | | | | | cysteine rich |
| | | | | | | do |
| 1133 | TIL | 2/5 | 758-813 | 42.4 | 5.6e−12 | Trypsin |
| | | | | | | Inhibitor like |
| | | | | | | cysteine rich |
| | | | | | | do |
| 1133 | TIL | 3/5 | 882-933 | 27.5 | 2.6e−07 | Trypsin |
| | | | | | | Inhibitor like |
| | | | | | | cysteine rich |
| | | | | | | do |
| 1133 | TIL | 4/5 | 1252-1302 | 34.9 | 1.3e−09 | Trypsin |
| | | | | | | Inhibitor like |
| | | | | | | cysteine rich |
| | | | | | | do |
| 1133 | vwa | 1/3 | 1383-1559 | 173.2 | 9.1e−50 | von Willebrand |
| | | | | | | factor type A |
| | | | | | | domain |
| 1133 | vwa | 2/3 | 1604-1770 | 203.6 | 1e−58 | von Willebrand |
| | | | | | | factor type A |
| | | | | | | domain |
| 1133 | vwa | 3/3 | 1797-1970 | 184.6 | 3.9e−53 | von Willebrand |
| | | | | | | factor type A |
| | | | | | | domain |
| 1133 | vwc | 1/12 | 456-494 | 14.5 | 0.0079 | von Willebrand |
| | | | | | | factor type C |
| | | | | | | domain |
| 1133 | vwc | 11/12 | 2688-2750 | 79.2 | 1.3e−21 | von Willebrand |
| | | | | | | factor type C |
| | | | | | | domain |
| 1133 | vwc | 3/12 | 935-974 | 25.8 | 4.1e−06 | von Willebrand |
| | | | | | | factor type C |
| | | | | | | domain |
| 1133 | vwc | 4/12 | 1305-1341 | 8.5 | 0.43 | von Willebrand |
| | | | | | | factor type C |
| | | | | | | domain |
| 1133 | vwc | 6/12 | 2363-2431 | 89.2 | 1.7e−24 | von Willebrand |
| | | | | | | factor type C |
| | | | | | | domain |
| 1133 | vwc | 9/12 | 2537-2600 | 90.6 | 6.4e−25 | von Willebrand |
| | | | | | | factor type C |
| | | | | | | domain |
| 1133 | vwd | 1/4 | 141-285 | 133.1 | 5.4e−37 | von Willebrand |
| | | | | | | factor type D |
| | | | | | | domain |
| 1133 | vwd | 2/4 | 494-647 | 183.4 | 2e−51 | von Willebrand |
| | | | | | | factor type D |
| | | | | | | domain |
| 1133 | vwd | 3/4 | 973-1119 | 153.1 | 1e−42 | von Willebrand |
| | | | | | | factor type D |
| | | | | | | domain |
| 1133 | vwd | 4/4 | 2056-2208 | 138.7 | 1.3e−38 | von Willebrand |
| | | | | | | factor type D |
| | | | | | | domain |
| 1134 | thiored | 1/1 | 88-151 | 44.0 | 6.3e−12 | Thioredoxin |
| 1137 | pkinase | 1/1 | 78-352 | 279.4 | 4.6e−80 | Protein kinase |
| | | | | | | domain |
| 1138 | FGGY | 1/1 | 84-111 | 11.3 | 0.051 | FGGY family of |
| | | | | | | carbohydrate |
| | | | | | | kinases, N- |
| | | | | | | termi |
| 1138 | HSP70 | 1/1 | 85-683 | 1382.0 | 0 | Hsp70 protein |
| 1139 | Anti_proliferat | 1/1 | 61-221 | 345.4 | 6.3e−100 | BTG1 family |
| 1140 | EMP70 | 1/1 | 11-72 | 125.3 | 9.5e−38 | Endomembrane |
| | | | | | | protein 70 |
| 1141 | Transglut_core | 1/1 | 338-427 | 185.8 | 7.1e−52 | Transglutaminase- |
| | | | | | | like |
| | | | | | | superfamily |
| 1141 | Transglutamin_C | 1/2 | 547-651 | 107.0 | 2.6e−30 | Transglutaminase |
| | | | | | | family, C- |
| | | | | | | terminal i |
| 1141 | Transglutamin_C | 2/2 | 659-756 | 108.8 | 7.2e−31 | Transglutaminase |
| | | | | | | family, C- |
| | | | | | | terminal i |
| 1141 | Transglutamin_N | 1/1 | 74-195 | 221.3 | 1.4e−64 | Transglutaminase |
| | | | | | | family |
| 1143 | PCI | 1/1 | 433-513 | 57.3 | 3.8e−15 | PCI domain |
| 1144 | PCI | 1/1 | 781-864 | 106.1 | 2.7e−29 | PCI domain |
| 1145 | RTX | 1/1 | 916-952 | 3.3 | 1 | RTX N-terminal |
| | | | | | | domain |
| 1145 | SMC_C | 1/1 | 1090-1291 | 410.9 | 5.9e−121 | SMC family, C- |
| | | | | | | terminal domain |
| 1145 | SMC_N | 1/1 | 93-271 | 289.6 | 3.8e−83 | RecF/RecN/SMC N |
| | | | | | | terminal domain |
| 1145 | SlyX | 1/1 | 340-351 | 3.6 | 0.4 | SlyX |
| 1146 | PGM_PMM | 1/1 | 512-622 | 159.5 | 1.3e−44 | Phosphoglucomutase/ |
| | | | | | | phosphomannomutase, |
| | | | | | | C- |
| 1146 | PGM_PMM_I | 1/1 | 82-233 | 186.9 | 3.3e−52 | Phosphoglucomutase/ |
| | | | | | | phosphomannomutase, |
| | | | | | | al |
| 1146 | PGM_PMM_II | 1/1 | 264-374 | 193.6 | 7e−62 | Phosphoglucomutase/ |
| | | | | | | phosphomannomutase, |
| | | | | | | al |
| 1146 | PGM_PMM_III | 1/1 | 376-491 | 174.2 | 1.7e−48 | Phosphoglucomutase/ |
| | | | | | | phosphomannomutase, |
| | | | | | | al |
| 1147 | C2 | 1/2 | 101-165 | 49.6 | 7.2e−13 | C2 domain |
| 1148 | ig | 1/2 | 99-169 | 29.0 | 2.3e−06 | Immunoglobulin |
| | | | | | | domain |
| 1148 | ig | 2/2 | 205-273 | 40.2 | 1.8e−09 | Immunoglobulin |
| | | | | | | domain |
| 1151 | efhand | 1/2 | 254-280 | 10.6 | 0.48 | EF hand |
| 1151 | efhand | 2/2 | 304-332 | 10.6 | 0.48 | EF hand |
| 1152 | SNase | 1/5 | 137-230 | 56.1 | 3.4e−14 | Staphylococcal |
| | | | | | | nuclease |
| | | | | | | homologue |
| 1152 | SNase | 2/5 | 303-392 | 34.5 | 3e−08 | Staphylococcal |
| | | | | | | nuclease |
| | | | | | | homologue |
| 1152 | SNase | 3/5 | 405-560 | 54.9 | 7e−14 | Staphylococcal |
| | | | | | | nuclease |
| | | | | | | homologue |
| 1152 | SNase | 4/5 | 589-724 | 77.1 | 5.5e−20 | Staphylococcal |
| | | | | | | nuclease |
| | | | | | | homologue |
| 1152 | SNase | 5/5 | 914-959 | 20.9 | 0.00017 | Staphylococcal |
| | | | | | | nuclease |
| | | | | | | homologue |
| 1152 | TUDOR | 1/1 | 741-861 | 137.2 | 3e−37 | Tudor domain |
| 1154 | filament | 1/1 | 133-440 | 581.6 | 4.8e−171 | Intermediate |
| | | | | | | filament |
| | | | | | | protein |
| 1155 | Calsequestrin | 1/1 | 27-62 | 5.4 | 0.4 | Calsequestrin |
| 1155 | EGF | 11/16 | 1557-1569 | 11.9 | 0.12 | EGF-like domain |
| 1155 | EGF | 13/16 | 3876-3908 | 44.1 | 9.5e−11 | EGF-like domain |
| 1155 | EGF | 14/16 | 3916-3949 | 31.3 | 3.7e−07 | EGF-like domain |
| 1155 | EGF | 15/16 | 4136-4168 | 31.5 | 3.4e−07 | EGF-like domain |
| 1155 | EGF | 16/16 | 4175-4203 | 24.9 | 2.5e−05 | EGF-like domain |
| 1155 | EGF | 3/16 | 758-770 | 10.9 | 0.22 | EGF-like domain |
| 1155 | EGF | 5/16 | 870-879 | 9.8 | 0.44 | EGF-like domain |
| 1155 | SEA | 1/1 | 106-220 | 105.7 | 2.6e−30 | SEA domain |
| 1155 | ig | 1/22 | 447-508 | 35.8 | 3e−08 | Immunoglobulin |
| | | | | | | domain |
| 1155 | ig | 10/22 | 2479-2533 | 44.3 | 1.3e−10 | Immunoglobulin |
| | | | | | | domain |
| 1155 | ig | 11/22 | 2576-2630 | 39.3 | 3.1e−09 | Immunoglobulin |
| | | | | | | domain |
| 1155 | ig | 12/22 | 2672-2726 | 41.8 | 6.5e−10 | Immunoglobulin |
| | | | | | | domain |
| 1155 | ig | 13/22 | 2769-2823 | 43.0 | 3e−10 | Immunoglobulin |
| | | | | | | domain |
| 1155 | ig | 14/22 | 2869-2923 | 40.8 | 1.2e−09 | Immunoglobulin |
| | | | | | | domain |
| 1155 | ig | 15/22 | 2967-3021 | 44.4 | 1.2e−10 | Immunoglobulin |
| | | | | | | domain |
| 1155 | ig | 16/22 | 3064-3121 | 26.2 | 1.3e−05 | Immunoglobulin |
| | | | | | | domain |
| 1155 | ig | 17/22 | 3155-3215 | 27.0 | 8e−06 | Immunoglobulin |
| | | | | | | domain |
| 1155 | ig | 18/22 | 3254-3308 | 31.6 | 4.4e−07 | Immunoglobulin |
| | | | | | | domain |
| 1155 | ig | 19/22 | 3341-3395 | 37.4 | 1.1e−08 | Immunoglobulin |
| | | | | | | domain |
| 1155 | ig | 2/22 | 1719-1776 | 32.6 | 2.2e−07 | Immunoglobulin |
| | | | | | | domain |
| 1155 | ig | 20/22 | 3442-3496 | 30.4 | 9.1e−07 | Immunoglobulin |
| | | | | | | domain |
| 1155 | ig | 21/22 | 3531-3585 | 31.0 | 6.5e−07 | Immunoglobulin |
| | | | | | | domain |
| 1155 | ig | 22/22 | 3617-3671 | 32.3 | 2.7e−07 | Immunoglobulin |
| | | | | | | domain |
| 1155 | ig | 3/22 | 1814-1870 | 33.9 | 1e−07 | Immunoglobulin |
| | | | | | | domain |
| 1155 | ig | 4/22 | 1908-1963 | 27.2 | 6.9e−06 | Immunoglobulin |
| | | | | | | domain |
| 1155 | ig | 5/22 | 1998-2055 | 45.0 | 8.5e−11 | Immunoglobulin |
| | | | | | | domain |
| 1155 | ig | 6/22 | 2093-2147 | 47.6 | 1.6e−11 | Immunoglobulin |
| | | | | | | domain |
| 1155 | ig | 7/22 | 2194-2248 | 45.0 | 8.5e−11 | Immunoglobulin |
| | | | | | | domain |
| 1155 | ig | 8/22 | 2287-2341 | 37.2 | 1.2e−08 | Immunoglobulin |
| | | | | | | domain |
| 1155 | ig | 9/22 | 2383-2437 | 42.5 | 4.2e−10 | Immunoglobulin |
| | | | | | | domain |
| 1155 | laminin_B | 1/3 | 623-757 | 78.0 | 2.1e−20 | Laminin B |
| | | | | | | (Domain IV) |
| 1155 | laminin_B | 2/3 | 1018-1152 | 136.4 | 5.2e−37 | Laminin B |
| | | | | | | (Domain IV) |
| 1155 | laminin_B | 3/3 | 1424-1556 | 133.7 | 3.4e−36 | Laminin B |
| | | | | | | (Domain IV) |
| 1155 | laminin_EGF | 10/15 | 1591-1638 | 61.5 | 4e−16 | Laminin EGF- |
| | | | | | | like (Domains |
| | | | | | | III and V) |
| 1155 | laminin_EGF | 11/15 | 1641-1696 | 29.9 | 4.6e−07 | Laminin EGF- |
| | | | | | | like (Domains |
| | | | | | | III and V) |
| 1155 | laminin_EGF | 12/15 | 3883-3910 | 9.4 | 0.37 | Laminin EGF- |
| | | | | | | like (Domains |
| | | | | | | III and V) |
| 1155 | laminin_EGF | 15/15 | 4191-4205 | 10.4 | 0.19 | Laminin EGF- |
| | | | | | | like (Domains |
| | | | | | | III and V) |
| 1155 | laminin_EGF | 2/15 | 792-839 | 61.8 | 3.2e−16 | Laminin EGF- |
| | | | | | | like (Domains |
| | | | | | | III and V) |
| 1155 | laminin_EGF | 3/15 | 842-897 | 37.8 | 2.5e−09 | Laminin EGF- |
| | | | | | | like (Domains |
| | | | | | | III and V) |
| 1155 | laminin_EGF | 4/15 | 919-949 | 22.6 | 5.8e−05 | Laminin EGF- |
| | | | | | | like (Domains |
| | | | | | | III and V) |
| 1155 | laminin_EGF | 5/15 | 1153-1184 | 10.7 | 0.16 | Laminin EGF- |
| | | | | | | like (Domains |
| | | | | | | III and V) |
| 1155 | laminin_EGF | 6/15 | 1187-1234 | 67.3 | 9e−18 | Laminin EGF- |
| | | | | | | like (Domains |
| | | | | | | III and V) |
| 1155 | laminin_EGF | 7/15 | 1237-1291 | 32.9 | 6.5e−08 | Laminin EGF- |
| | | | | | | like (Domains |
| | | | | | | III and V) |
| 1155 | laminin_EGF | 8/15 | 1303-1350 | 55.4 | 2.3e−14 | Laminin EGF- |
| | | | | | | like (Domains |
| | | | | | | III and V) |
| 1155 | laminin_EGF | 9/15 | 1557-1576 | 14.6 | 0.012 | Laminin EGF- |
| | | | | | | like (Domains |
| | | | | | | III and V) |
| 1155 | laminin_G | 1/3 | 3720-3859 | 111.4 | 2e−30 | Laminin G |
| | | | | | | domain |
| 1155 | laminin_G | 2/3 | 3985-4116 | 128.9 | 2.2e−35 | Laminin G |
| | | | | | | domain |
| 1155 | laminin_G | 3/3 | 4262-4395 | 131.0 | 5.5e−36 | Laminin G |
| | | | | | | domain |
| 1155 | ldl_recept_a | 1/4 | 223-262 | 47.2 | 8.4e−13 | Low-density |
| | | | | | | lipoprotein |
| | | | | | | receptor domain |
| 1155 | ldl_recept_a | 2/4 | 309-347 | 54.8 | 4e−15 | Low-density |
| | | | | | | lipoprotein |
| | | | | | | receptor domain |
| 1155 | ldl_recept_a | 3/4 | 349-387 | 57.1 | 8.3e−16 | Low-density |
| | | | | | | lipoprotein |
| | | | | | | receptor domain |
| 1155 | ldl_recept_a | 4/4 | 392-431 | 44.0 | 8e−12 | Low-density |
| | | | | | | lipoprotein |
| | | | | | | receptor domain |
| 1158 | PX | 1/1 | 134-173 | 20.1 | 0.00098 | PX domain |
| 1158 | pkinase | 1/1 | 210-467 | 304.7 | 1.1e−87 | Protein kinase |
| | | | | | | domain |
| 1158 | pkinase_C | 1/1 | 468-542 | 56.0 | 5.6e−16 | Protein kinase |
| | | | | | | C terminal |
| | | | | | | domain |
| 1161 | DUF260 | 1/1 | 56-82 | 3.3 | 0.95 | Protein of |
| | | | | | | unknown |
| | | | | | | function DUF260 |
| 1163 | K-box | 1/2 | 83-110 | 6.9 | 1 | K-box region |
| 1163 | Retrotrans_gag | 1/1 | 193-286 | 78.5 | 2.9e−21 | Retrotransposon |
| | | | | | | gag protein |
| 1163 | zf-CCHC | 1/1 | 367-384 | 16.7 | 0.011 | Zinc knuckle |
| 1164 | 7tm_2 | 1/1 | 474-723 | 376.5 | 6.9e−112 | 7 transmembrane |
| | | | | | | receptor |
| | | | | | | (Secretin |
| | | | | | | family) |
| 1164 | DsbB | 1/1 | 475-504 | 6.7 | 0.91 | Disulfide bond |
| | | | | | | formation |
| | | | | | | protein DsbB |
| 1164 | EGF | 2/3 | 91-120 | 12.5 | 0.077 | EGF-like domain |
| 1164 | EGF | 3/3 | 143-175 | 16.2 | 0.0072 | EGF-like domain |
| 1164 | GPS | 1/1 | 421-472 | 84.0 | 7.2e−22 | Latrophilin/CL- |
| | | | | | | 1-like GPS |
| | | | | | | domain |
| 1165 | sugar_tr | 1/1 | 39-492 | 661.0 | 6.1e−195 | Sugar (and |
| | | | | | | other) |
| | | | | | | transporter |
| 1167 | MBD | 1/1 | 14-67 | 86.9 | 2.1e−22 | Methyl-CpG |
| | | | | | | binding domain |
| 1168 | aa_permeases | 1/1 | 356-408 | 8.3 | 0.18 | Amino acid |
| | | | | | | permease |
| 1170 | Reticulon | 1/1 | 223-414 | 368.1 | 2e−112 | Reticulon |
| 1172 | FKBP | 1/1 | 41-135 | 179.4 | 9.1e−54 | FKBP-type |
| | | | | | | peptidyl-prolyl |
| | | | | | | cis-trans |
| | | | | | | isomeras |
| 1174 | abhydrolase | 1/2 | 47-104 | 28.9 | 7e−08 | alpha/beta |
| | | | | | | hydrolase fold |
| 1174 | abhydrolase | 2/2 | 120-186 | 7.6 | 0.24 | alpha/beta |
| | | | | | | hydrolase fold |
| 1174 | abhydrolase_2 | 1/1 | 173-194 | 7.3 | 0.61 | Phospholipase/C |
| | | | | | | arboxylesterase |
| 1176 | DUF357 | 1/1 | 449-465 | 8.6 | 0.48 | Protein of |
| | | | | | | unknown |
| | | | | | | function |
| | | | | | | (DUF357) |
| 1176 | Furin-like | 1/1 | 627-644 | 8.0 | 0.23 | Furin-like |
| | | | | | | cysteine rich |
| | | | | | | region |
| 1176 | P | 1/1 | 470-601 | 250.4 | 2.5e−71 | Proprotein |
| | | | | | | convertase P- |
| | | | | | | domain |
| 1176 | Peptidase_S8 | 1/1 | 153-458 | 436.3 | 2.8e−127 | Subtilase |
| | | | | | | family |
| 1176 | VSP | 2/2 | 667-690 | 6.4 | 0.56 | 1/2 242 |
| | | | | | | 266..129 |
| 1179 | Exostosin | 1/1 | 217-503 | 327.6 | 1.4e−94 | Exostosin |
| | | | | | | family |
| 1182 | COLFI | 1/1 | 1661-1872 | 488.9 | 1.5e−190 | Fibrillar |
| | | | | | | collagen C- |
| | | | | | | terminal domain |
| 1182 | Collagen | 10/20 | 1009-1068 | 53.8 | 2.6e−13 | Collagen triple |
| | | | | | | helix repeat |
| | | | | | | (20 copies) |
| 1182 | Collagen | 11/20 | 1069-1104 | 26.4 | 9.2e−06 | Collagen triple |
| | | | | | | helix repeat |
| | | | | | | (20 copies) |
| 1182 | Collagen | 12/20 | 1105-1161 | 42.5 | 3.5e−10 | Collagen triple |
| | | | | | | helix repeat |
| | | | | | | (20 copies) |
| 1182 | Collagen | 13/20 | 1162-1221 | 54.6 | 1.6e−13 | Collagen triple |
| | | | | | | helix repeat |
| | | | | | | (20 copies) |
| 1182 | Collagen | 14/20 | 1222-1281 | 54.2 | 2e−13 | Collagen triple |
| | | | | | | helix repeat |
| | | | | | | (20 copies) |
| 1182 | Collagen | 15/20 | 1285-1344 | 42.9 | 2.6e−10 | Collagen triple |
| | | | | | | helix repeat |
| | | | | | | (20 copies) |
| 1182 | Collagen | 16/20 | 1345-1404 | 55.5 | 9.3e−14 | Collagen triple |
| | | | | | | helix repeat |
| | | | | | | (20 copies) |
| 1182 | Collagen | 17/20 | 1405-1457 | 34.2 | 6.5e−08 | Collagen triple |
| | | | | | | helix repeat |
| | | | | | | (20 copies) |
| 1182 | Collagen | 18/20 | 1459-1518 | 57.2 | 3.1e−14 | Collagen triple |
| | | | | | | helix repeat |
| | | | | | | (20 copies) |
| 1182 | Collagen | 19/20 | 1528-1587 | 59.0 | 9.7e−15 | Collagen triple |
| | | | | | | helix repeat |
| | | | | | | (20 copies) |
| 1182 | Collagen | 2/20 | 505-553 | 41.4 | 6.8e−10 | Collagen triple |
| | | | | | | helix repeat |
| | | | | | | (20 copies) |
| 1182 | Collagen | 20/20 | 1588-1607 | 11.1 | 0.14 | Collagen triple |
| | | | | | | helix repeat |
| | | | | | | (20 copies) |
| 1182 | Collagen | 3/20 | 593-648 | 34.1 | 6.9e−08 | Collagen triple |
| | | | | | | helix repeat |
| | | | | | | (20 copies) |
| 1182 | Collagen | 4/20 | 649-708 | 59.8 | 6e−15 | Collagen triple |
| | | | | | | helix repeat |
| | | | | | | (20 copies) |
| 1182 | Collagen | 5/20 | 709-768 | 58.8 | 1.1e−14 | Collagen triple |
| | | | | | | helix repeat |
| | | | | | | (20 copies) |
| 1182 | Collagen | 6/20 | 769-828 | 40.8 | 9.7e−10 | Collagen triple |
| | | | | | | helix repeat |
| | | | | | | (20 copies) |
| 1182 | Collagen | 7/20 | 832-887 | 34.5 | 5.5e−08 | Collagen triple |
| | | | | | | helix repeat |
| | | | | | | (20 copies) |
| 1182 | Collagen | 8/20 | 889-948 | 41.9 | 4.9e−10 | Collagen triple |
| | | | | | | helix repeat |
| | | | | | | (20 copies) |
| 1182 | Collagen | 9/20 | 949-1008 | 57.9 | 2e−14 | Collagen triple |
| | | | | | | helix repeat |
| | | | | | | (20 copies) |
| 1182 | TSPN | 1/1 | 75-266 | 290.0 | 1.7e−83 | Thrombospondin |
| | | | | | | N-terminal - |
| | | | | | | like domain |
| 1182 | laminin_G | 1/1 | 195-218 | 14.6 | 0.0071 | Laminin G |
| | | | | | | domain |
| 1185 | fibrinogen_C | 1/2 | 235-335 | 156.5 | 1e−43 | Fibrinogen beta |
| | | | | | | and gamma |
| | | | | | | chains, C-term |
| 1185 | fibrinogen_C | 2/2 | 377-451 | 67.0 | 3.5e−18 | Fibrinogen beta |
| | | | | | | and gamma |
| | | | | | | chains, C-term |
| 1187 | SH3 | 1/1 | 335-389 | 78.3 | 6.8e−20 | SH3 domain |
| 1188 | AT_hook | 1/1 | 73-85 | 8.9 | 1 | AT hook motif |
| 1188 | Vinculin | 1/1 | 43-891 | 1728.4 | 0 | Vinculin family |
| 1189 | annexin | 1/2 | 13-67 | 67.1 | 1e−16 | Annexin |
| 1189 | annexin | 2/2 | 75-142 | 109.4 | 6.9e−29 | Annexin |
| 1190 | Heme_oxygenase | 1/1 | 35-272 | 639.9 | 1.4e−188 | Heme oxygenase |
| 1192 | DED | 1/1 | 35-112 | 19.8 | 0.00037 | Death effector |
| | | | | | | domain |
| 1193 | proteasome | 1/1 | 38-226 | 230.9 | 1.9e−65 | Proteasome A- |
| | | | | | | type and B-type |
| 1194 | adh_short | 1/1 | 88-380 | 244.7 | 1.3e−69 | short chain |
| | | | | | | dehydrogenase |
| 1195 | V-ATPase_G | 1/1 | 13-118 | 205.7 | 6.9e−58 | Vacuolar (H+)- |
| | | | | | | ATPase G |
| | | | | | | subunit |
| 1197 | BTB | 2/2 | 490-594 | 81.6 | 4.7e−21 | BTB/POZ domain |
| 1197 | ras | 2/2 | 231-250 | 6.2 | 0.92 | Ras family |
| 1198 | AMP-binding | 1/1 | 1-245 | 224.3 | 1.8e−63 | AMP-binding |
| | | | | | | enzyme |
| 1199 | gla | 1/1 | 39-80 | 67.3 | 2.6e−19 | Vitamin K- |
| | | | | | | dependent |
| | | | | | | carboxylation/gamma- |
| 1200 | PDZ | 1/2 | 119-198 | 49.1 | 1.6e−12 | PDZ domain |
| | | | | | | (Also known as |
| | | | | | | DHR or GLGF) |
| 1200 | PDZ | 2/2 | 203-277 | 25.2 | 1.1e−05 | PDZ domain |
| | | | | | | (Also known as |
| | | | | | | DHR or GLGF) |
| 1201 | EPH_lbd | 1/1 | 20-200 | 399.0 | 3.8e−117 | Ephrin receptor |
| | | | | | | ligand binding |
| | | | | | | domain |
| 1201 | SAM | 1/1 | 908-972 | 93.9 | 3.6e−25 | SAM domain |
| | | | | | | (Sterile alpha |
| | | | | | | motif) |
| 1201 | fn3 | 1/2 | 327-417 | 39.0 | 8.6e−10 | Fibronectin |
| | | | | | | type III domain |
| 1201 | fn3 | 2/2 | 437-522 | 68.4 | 2.7e−18 | Fibronectin |
| | | | | | | type III domain |
| 1201 | pkinase | 1/1 | 618-874 | 256.1 | 4.6e−73 | Protein kinase |
| | | | | | | domain |
| 1202 | GTF2I | 1/6 | 230-305 | 172.6 | 6.9e−55 | GTF2I-like |
| | | | | | | repeat |
| 1202 | GTF2I | 2/6 | 438-513 | 172.9 | 5.3e−55 | GTF2I-like |
| | | | | | | repeat |
| 1202 | GTF2I | 3/6 | 543-618 | 182.0 | 6.2e−58 | GTF2I-like |
| | | | | | | repeat |
| 1202 | GTF2I | 4/6 | 648-723 | 183.6 | 1.8e−58 | GTF2I-like |
| | | | | | | repeat |
| 1202 | GTF2I | 5/6 | 810-885 | 177.3 | 2e−56 | GTF2I-like |
| | | | | | | repeat |
| 1202 | GTF2I | 6/6 | 945-1020 | 165.2 | 1.7e−52 | GTF2I-like |
| | | | | | | repeat |
| 1202 | ribonuc_red_sm | 1/1 | 869-898 | 6.0 | 0.57 | Ribonucleotide |
| | | | | | | reductase, |
| | | | | | | small chain |
| 1203 | GRAM | 1/1 | 13-96 | 65.5 | 1.1e−17 | GRAM domain |
| 1204 | FlpD | 1/1 | 607-622 | 5.5 | 1 | Methyl- |
| | | | | | | viologen- |
| | | | | | | reducing |
| | | | | | | hydrogenase, |
| 1204 | crystall | 1/6 | 971-997 | 23.0 | 0.00046 | Beta/Gamma |
| | | | | | | crystallin |
| 1204 | crystall | 2/6 | 1072-1119 | 49.7 | 6.5e−11 | Beta/Gamma |
| | | | | | | crystallin |
| 1204 | crystall | 3/6 | 1133-1126 | 95.3 | 1.2e−24 | Beta/Gamma |
| | | | | | | crystallin |
| 1204 | crystall | 4/6 | 1234-1317 | 62.7 | 8.2e−15 | Beta/Gamma |
| | | | | | | crystallin |
| 1204 | crystall | 5/6 | 1330-1409 | 59.9 | 5.5e−14 | Beta/Gamma |
| | | | | | | crystallin |
| 1204 | crystall | 6/6 | 1417-1497 | 84.0 | 3.1e−21 | Beta/Gamma |
| | | | | | | crystallin |
| 1204 | gpdh | 1/1 | 498-508 | 5.1 | 0.055 | Glyceraldehyde |
| | | | | | | 3-phosphate |
| | | | | | | dehydrogena |
| 1205 | hormone | 1/1 | 2-143 | 124.6 | 6.8e−46 | Somatotropin |
| | | | | | | hormone family |
| 1206 | histone | 1/1 | 30-146 | 188.4 | 1.1e−52 | Core histone |
| | | | | | | H2A/H2B/H3/H4 |
| 1208 | Gal-bind_lectin | 1/1 | 67-198 | 277.2 | 2.1e−79 | Galactoside- |
| | | | | | | binding lectin |
| 1209 | ENV_polyprotein | 1/1 | 1097-1140 | 63.8 | 2.4e−18 | ENV polyprotein |
| | | | | | | (coat |
| | | | | | | polyprotein) |
| 1209 | rnaseH | 1/1 | 662-769 | 70.8 | 2.1e−18 | RNase H |
| 1209 | rve | 1/1 | 885-1039 | 126.3 | 3.5e−35 | Integrase core |
| | | | | | | domain |
| 1209 | rvp | 1/1 | 20-117 | 72.3 | 7.6e−19 | Retroviral |
| | | | | | | aspartyl |
| | | | | | | protease |
| 1209 | rvt | 1/1 | 203-380 | 110.8 | 2.1e−29 | Reverse |
| | | | | | | transcriptase |
| | | | | | | (RNA-dependent |
| 1211 | Peptidase_C1 | 1/1 | 302-513 | 353.9 | 2.6e−105 | Papain family |
| | | | | | | cysteine |
| | | | | | | protease |
| 1211 | gpdh | 1/1 | 430-439 | 3.6 | 0.25 | Glyceraldehyde |
| | | | | | | 3-phosphate |
| | | | | | | dehydrogenase |
| 1212 | ANATO | 1/1 | 63-71 | 7.4 | 0.033 | Anaphylotoxin- |
| | | | | | | like domain |
| 1214 | kazal | 1/1 | 18-56 | 22.2 | 0.00062 | Kazal-type |
| | | | | | | serine protease |
| | | | | | | inhibitor |
| | | | | | | domain |
| 1215 | p34-Arc | 1/1 | 16-73 | 35.7 | 1e−08 | Arp2/3 complex, |
| | | | | | | 34 kD subunit |
| | | | | | | p34-Arc |
| 1216 | EGF | 2/3 | 64-100 | 25.9 | 1.3e−05 | EGF-like domain |
| 1216 | EGF | 3/3 | 106-140 | 13.2 | 0.05 | EGF-like domain |
| 1217 | kazal | 1/1 | 39-87 | 36.5 | 1.4e−07 | Kazal-type |
| | | | | | | serine protease |
| | | | | | | inhibitor |
| | | | | | | domain |
| 1218 | filament | 1/1 | 13-76 | 134.7 | 1.6e−36 | Intermediate |
| | | | | | | filament |
| | | | | | | protein |
| 1219 | pkinase | 1/1 | 99-361 | 290.7 | 1.8e−83 | Protein kinase |
| | | | | | | domain |
| 1221 | transmembrane4 | 1/1 | 22-70 | 52.3 | 1.5e−15 | Tetraspanin |
| | | | | | | family |
| 1222 | ferritin | 1/1 | 1-109 | 177.7 | 1.8e−49 | Ferritin-like |
| | | | | | | domain |
| 1223 | DUF260 | 1/1 | 33-68 | 4.8 | 0.23 | Protein of |
| | | | | | | unknown |
| | | | | | | function DUF260 |
| 1223 | bZIP | 1/1 | 16-51 | 10.2 | 0.22 | bZIP |
| | | | | | | transcription |
| | | | | | | factor |
| 1224 | hormone | 1/1 | 4-107 | 119.8 | 4.2e−44 | Somatotropin |
| | | | | | | hormone family |
| 1225 | sushi | 1/1 | 16-43 | 27.4 | 1.7e−05 | Sushi domain |
| | | | | | | (SCR repeat) |
| 1227 | L15 | 1/1 | 66-92 | 44.5 | 3.2e−10 | Ribosomal |
| | | | | | | protein L15 |
| 1229 | Dynein_heavy | 1/1 | 11-127 | 91.5 | 2.6e−26 | Dynein heavy |
| | | | | | | chain |
| 1231 | dCMP_cyt_deam | 1/1 | 34-140 | 98.5 | 1.3e−26 | Cytidine and |
| | | | | | | deoxycytidylate |
| | | | | | | deaminase |
| 1232 | adh_short | 1/1 | 38-146 | 99.8 | 3.4e−27 | short chain |
| | | | | | | dehydrogenase |
| 1233 | ldh | 1/1 | 44-129 | 143.7 | 3.2e−39 | lactate/malate |
| | | | | | | dehydrogenase, |
| | | | | | | NAD binding d |
| 1234 | HMG_box | 1/2 | 1-66 | 59.4 | 4.5e−15 | HMG (high |
| | | | | | | mobility group) |
| | | | | | | box |
| 1234 | HMG_box | 2/2 | 82-145 | 88.0 | 4.4e−23 | HMG (high |
| | | | | | | mobility group) |
| | | | | | | box |
| 1236 | EGF | 1/3 | 2-27 | 13.7 | 0.037 | EGF-like domain |
| 1236 | EGF | 2/3 | 33-68 | 30.1 | 8.6e−07 | EGF-like domain |
| 1236 | EGF | 3/3 | 74-110 | 28.1 | 3.1e−06 | EGF-like domain |
| 1236 | PSI | 1/1 | 19-47 | 6.7 | 0.78 | Plexin repeat |
| 1237 | cystatin | 1/1 | 2-93 | 124.3 | 5.4e−35 | Cystatin domain |
| 1239 | cadherin | 1/1 | 36-62 | 9.5 | 0.22 | Cadherin domain |
| 1240 | bZIP | 1/1 | 226-282 | 16.8 | 0.003 | bZIP |
| | | | | | | transcription |
| | | | | | | factor |
| 1242 | IF_tail | 1/1 | 1-85 | 167.1 | 1.9e−46 | Intermediate |
| | | | | | | filament tail |
| | | | | | | domain |
| 1244 | Ribosomal_S15 | 1/1 | 88-149 | 14.6 | 0.0094 | Ribosomal |
| | | | | | | protein S15 |
| 1246 | ldh | 1/1 | 1-42 | 82.0 | 4.4e−21 | lactate/malate |
| | | | | | | dehydrogenase, |
| | | | | | | NAD binding do |
| 1246 | ldh_C | 1/1 | 44-130 | 163.1 | 4.9e−45 | lactate/malate |
| | | | | | | dehydrogenase, |
| | | | | | | alpha/beta C-t |
| 1249 | W2 | 1/1 | 35-99 | 97.3 | 5.1e−29 | eIF4- |
| | | | | | | gamma/eIF5/eIF2- |
| | | | | | | epsilon |
| 1251 | Pep_M12B_propep | 1/1 | 120-198 | 75.4 | 2.7e−20 | Reprolysin |
| | | | | | | family |
| | | | | | | propeptide |
| 1251 | Reprolysin | 2/3 | 352-449 | 31.3 | 5.6e−07 | Reprolysin |
| | | | | | | (M12B) family |
| | | | | | | zinc metallo |
| 1251 | Tfb2 | 1/1 | 312-330 | 3.3 | 0.1 | Transcription |
| | | | | | | factor Tfb2 |
| 1251 | tsp_1 | 2/4 | 595-645 | 43.1 | 2.3e−11 | Thrombospondin |
| | | | | | | type 1 domain |
| 1251 | tsp_1 | 3/4 | 888-941 | 12.4 | 0.032 | Thrombospondin |
| | | | | | | type 1 domain |
| 1251 | tsp_1 | 4/4 | 947-998 | 24.6 | 7.2e−06 | Thrombospondin |
| | | | | | | type 1 domain |
| 1253 | ANATO | 1/1 | 60-66 | 4.4 | 0.46 | Anaphylotoxin- |
| | | | | | | like domain |
| 1255 | UPF0005 | 1/1 | 15-144 | 58.8 | 6.8e−16 | Uncharacterized |
| | | | | | | protein family |
| | | | | | | UPF0005 |
| 1256 | actin | 1/1 | 1-145 | 358.0 | 1.5e−108 | Actin |
| 1258 | OSCP | 1/1 | 37-209 | 257.9 | 1.9e−75 | ATP synthase |
| | | | | | | delta (OSCP) |
| | | | | | | subunit |
| 1259 | PA28_alpha | 1/1 | 9-72 | 135.5 | 9.4e−37 | Proteasome |
| | | | | | | activator pa28 |
| | | | | | | alpha subuni |
| 1259 | PA28_beta | 1/1 | 90-239 | 387.7 | 1.1e−112 | Proteasome |
| | | | | | | activator pa28 |
| | | | | | | beta subunit |
| 1260 | WD40 | 1/5 | 166-205 | 31.0 | 5.2e−07 | WD domain, G- |
| | | | | | | beta repeat |
| 1260 | WD40 | 2/5 | 216-255 | 24.2 | 4.5e−05 | WD domain, G- |
| | | | | | | beta repeat |
| 1260 | WD40 | 3/5 | 262-301 | 40.4 | 1.1e−09 | WD domain, G- |
| | | | | | | beta repeat |
| 1260 | WD40 | 4/5 | 306-345 | 33.9 | 7.8e−08 | WD domain, G- |
| | | | | | | beta repeat |
| 1260 | WD40 | 5/5 | 363-402 | 28.6 | 2.5e−06 | WD domain, G- |
| | | | | | | beta repeat |
| 1261 | WD40 | 1/5 | 6-38 | 13.2 | 0.059 | WD domain, G- |
| | | | | | | beta repeat |
| 1261 | WD40 | 2/5 | 42-80 | 41.8 | 4.4e−10 | WD domain, G- |
| | | | | | | beta repeat |
| 1261 | WD40 | 4/5 | 178-216 | 28.6 | 2.5e−06 | WD domain, G- |
| | | | | | | beta repeat |
| 1261 | WD40 | 5/5 | 275-313 | 24.1 | 4.6e-05 | WD domain, G- |
| | | | | | | beta repeat |
| 1262 | Big_1 | 1/3 | 1337-1373 | 8.9 | 0.16 | Bacterial Ig- |
| | | | | | | like domain |
| | | | | | | (group 1) |
| 1262 | CH | 1/2 | 72-177 | 97.6 | 1.3e−25 | Calponin |
| | | | | | | homology (CH) |
| | | | | | | domain |
| 1262 | CH | 2/2 | 195-297 | 78.2 | 3.2e−20 | Calponin |
| | | | | | | homology (CH) |
| | | | | | | domain |
| 1262 | Filamin | 1/24 | 306-399 | 127.4 | 2e−37 | Filamin/ABP280 |
| | | | | | | repeat |
| 1262 | Filamin | 10/24 | 1185-1274 | 96.0 | 7.1e−28 | Filamin/ABP280 |
| | | | | | | repeat |
| 1262 | Filamin | 11/24 | 1280-1374 | 120.5 | 2.5e−35 | Filamin/ABP280 |
| | | | | | | repeat |
| 1262 | Filamin | 12/24 | 1380-1467 | 139.6 | 3.8e−41 | Filamin/ABP280 |
| | | | | | | repeat |
| 1262 | Filamin | 13/24 | 1473-1563 | 137.8 | 1.3e−40 | Filamin/ABP280 |
| | | | | | | repeat |
| 1262 | Filamin | 14/24 | 1569-1660 | 124.8 | 1.2e−36 | Filamin/ABP280 |
| | | | | | | repeat |
| 1262 | Filamin | 15/24 | 1666-1756 | 121.9 | 9.1e−36 | Filamin/ABP280 |
| | | | | | | repeat |
| 1262 | Filamin | 16/24 | 1812-1868 | 76.6 | 5.4e−22 | Filamin/ABP280 |
| | | | | | | repeat |
| 1262 | Filamin | 17/24 | 1873-1960 | 145.4 | 6.6e−43 | Filamin/ABP280 |
| | | | | | | repeat |
| 1262 | Filamin | 18/24 | 1971-2046 | 63.2 | 6.8e−18 | Filamin/ABP280 |
| | | | | | | repeat |
| 1262 | Filamin | 19/24 | 2054-2141 | 135.8 | 5.4e−40 | Filamin/ABP280 |
| | | | | | | repeat |
| 1262 | Filamin | 2/24 | 406-498 | 122.7 | 5.3e−36 | Filamin/ABP280 |
| | | | | | | repeat |
| 1262 | Filamin | 20/24 | 2142-2237 | 78.2 | 1.8e−22 | Filamin/ABP280 |
| | | | | | | repeat |
| 1262 | Filamin | 21/24 | 2245-2332 | 154.6 | 1e−45 | Filamin/ABP280 |
| | | | | | | repeat |
| 1262 | Filamin | 22/24 | 2339-2427 | 95.4 | 1.1e−27 | Filamin/ABP280 |
| | | | | | | repeat |
| 1262 | Filamin | 23/24 | 2436-2523 | 103.7 | 3.2e−30 | Filamin/ABP280 |
| | | | | | | repeat |
| 1262 | Filamin | 24/24 | 2564-2653 | 112.4 | 7.5e−33 | Filamin/ABP280 |
| | | | | | | repeat |
| 1262 | Filamin | 3/24 | 504-595 | 129.1 | 6.1e−38 | Filamin/ABP280 |
| | | | | | | repeat |
| 1262 | Filamin | 4/24 | 601-688 | 106.5 | 4.4e−31 | Filamin/ABP280 |
| | | | | | | repeat |
| 1262 | Filamin | 5/24 | 697-788 | 94.3 | 2.4e−27 | Filamin/ABP280 |
| | | | | | | repeat |
| 1262 | Filamin | 6/24 | 794-891 | 128.8 | 7.3e−38 | Filamin/ABP280 |
| | | | | | | repeat |
| 1262 | Filamin | 7/24 | 897-990 | 127.9 | 1.4e−37 | Filamin/ABP280 |
| | | | | | | repeat |
| 1262 | Filamin | 8/24 | 996-1086 | 86.5 | 5.7e−25 | Filamin/ABP280 |
| | | | | | | repeat |
| 1262 | Filamin | 9/24 | 1092-1179 | 163.5 | 2.1e−48 | Filamin/ABP280 |
| | | | | | | repeat |
| 1262 | Lipase_3 | 1/1 | 977-992 | 6.2 | 0.79 | Lipase (class |
| | | | | | | 3) |
| 1263 | CUE | 1/1 | 296-337 | 45.9 | 5.6e−12 | CUE domain |
| 1264 | fibrinogen_C | 1/1 | 28-242 | 252.8 | 4.6e−72 | Fibrinogen beta |
| | | | | | | and gamma |
| | | | | | | chains, C-term |
| 1265 | HLH | 1/1 | 80-132 | 74.3 | 1.7e−18 | Helix-loop- |
| | | | | | | helix DNA- |
| | | | | | | binding domain |
| 1266 | transmembrane4 | 1/1 | 11-221 | 270.7 | 2.9e−84 | Tetraspanin |
| | | | | | | family |
| 1267 | HSP70 | 1/1 | 39-91 | 5.5 | 0.5 | Hsp70 protein |
| 1267 | PC_rep | 1/8 | 409-442 | 18.5 | 0.0018 | Proteasome/cyclosome |
| | | | | | | repeat |
| 1267 | PC_rep | 2/8 | 443-479 | 31.3 | 4e−07 | Proteasome/cyclosome |
| | | | | | | repeat |
| 1267 | PC_rep | 3/8 | 480-514 | 30.5 | 6.9e−07 | Proteasome/cyclosome |
| | | | | | | repeat |
| 1267 | PC_rep | 4/8 | 517-551 | 17.8 | 0.0029 | Proteasome/cyclosome |
| | | | | | | repeat |
| 1267 | PC_rep | 5/8 | 560-589 | 11.4 | 0.19 | Proteasome/cyclosome |
| | | | | | | repeat |
| 1267 | PC_rep | 7/8 | 692-723 | 17.5 | 0.0035 | Proteasome/cyclosome |
| | | | | | | repeat |
| 1267 | PC_rep | 8/8 | 724-757 | 19.9 | 0.00071 | Proteasome/cyclosome |
| | | | | | | repeat |
| 1268 | annexin | 1/4 | 18-85 | 111.4 | 1.8e−29 | Annexin |
| 1268 | annexin | 2/4 | 90-157 | 105.7 | 9e−28 | Annexin |
| 1268 | annexin | 3/4 | 173-241 | 108.0 | 1.8e−28 | Annexin |
| 1268 | annexin | 4/4 | 249-316 | 122.3 | 8.8e−33 | Annexin |
| 1269 | PID | 1/1 | 50-176 | 162.3 | 8e−45 | Phosphotyrosine |
| | | | | | | interaction |
| | | | | | | domain |
| | | | | | | (PTB/PID) |
| 1270 | globin | 1/1 | 3-147 | 221.7 | 1.1e−62 | Globin |
| 1273 | Connexin43 | 1/1 | 301-320 | 39.9 | 8.9e−10 | Gap junction |
| | | | | | | alpha-1 protein |
| | | | | | | (Cx43) |
| 1273 | SMC_N | 1/1 | 133-144 | 6.5 | 0.73 | RecF/RecN/SMC N |
| | | | | | | terminal domain |
| 1273 | connexin | 1/1 | 9-239 | 576.9 | 1.2e−169 | Connexin |
| 1274 | trypsin | 1/1 | 9-231 | 318.0 | 2.6e−100 | Trypsin |
| 1276 | MAGE | 1/1 | 471-592 | 119.5 | 2e−32 | MAGE family |
| 1276 | SpoA | 1/1 | 518-535 | 8.3 | 0.88 | Surface |
| | | | | | | presentation of |
| | | | | | | antigens (SPOA) |
| 1277 | SH3 | 1/1 | 68-124 | 42.0 | 6.7e−10 | SH3 domain |
| 1279 | ATP1G1_PLM_MAT8 | 1/1 | 20-75 | 128.7 | 1.7e−38 | ATP1G1/PLM/MAT8 |
| | | | | | | family |
| 1280 | PAP2 | 1/1 | 100-251 | 179.5 | 5.5e−50 | PAP2 |
| | | | | | | superfamily |
| 1281 | cpn60_TCP1 | 1/1 | 39-529 | 628.8 | 3.1e−185 | TCP-1/cpn60 |
| | | | | | | chaperonin |
| | | | | | | family |
| 1282 | thiored | 1/2 | 24-132 | 167.5 | 3.5e−49 | Thioredoxin |
| 1282 | thiored | 2/2 | 159-270 | 187.1 | 4.3e−55 | Thioredoxin |
| 1283 | Astacin | 1/1 | 561-572 | 7.5 | 0.22 | Astacin |
| | | | | | | (Peptidase |
| | | | | | | family M12A) |
| 1283 | notch | 1/3 | 416-441 | 13.5 | 0.09 | Notch (DSL) |
| | | | | | | domain |
| 1283 | notch | 2/3 | 451-474 | 13.2 | 0.11 | Notch (DSL) |
| | | | | | | domain |
| 1283 | sushi | 1/4 | 1215-1280 | 23.2 | 0.00022 | Sushi domain |
| | | | | | | (SCR repeat) |
| 1283 | sushi | 2/4 | 1305-1342 | 15.8 | 0.02 | Sushi domain |
| | | | | | | (SCR repeat) |
| 1283 | sushi | 3/4 | 1346-1410 | 32.5 | 7.9e−07 | Sushi domain |
| | | | | | | (SCR repeat) |
| 1283 | sushi | 4/4 | 1415-1471 | 23.2 | 0.00022 | Sushi domain |
| | | | | | | (SCR repeat) |
| 1284 | BAG | 1/1 | 175-255 | 84.6 | 1.4e−22 | BAG domain |
| 1284 | ThiI | 1/1 | 206-223 | 7.2 | 0.53 | Thiamine |
| | | | | | | biosynthesis |
| | | | | | | protein (ThiI) |
| 1284 | Tropomyosin | 1/1 | 160-183 | 8.4 | 0.2 | Tropomyosin |
| 1284 | ubiquitin | 1/1 | 99-151 | 27.6 | 3.3e−06 | Ubiquitin |
| | | | | | | family |
| 1285 | bZIP | 1/1 | 186-250 | 35.3 | 1.6e−08 | bZIP |
| | | | | | | transcription |
| | | | | | | factor |
| 1286 | HSP70 | 1/1 | 30-635 | 1403.0 | 0 | Hsp70 protein |
| 1286 | Hydantoinase_A | 1/1 | 213-236 | 7.6 | 0.13 | Hydantoinase/oxoprolinase |
| 1287 | DSBA | 1/2 | 49-69 | 7.1 | 0.73 | DSBA |
| | | | | | | oxidoreductase |
| 1287 | DSBA | 2/2 | 357-426 | 7.8 | 0.48 | DSBA |
| | | | | | | oxidoreductase |
| 1287 | thiored | 1/2 | 24-133 | 190.7 | 3.5e−56 | Thioredoxin |
| 1287 | thiored | 2/2 | 375-485 | 183.5 | 5.2e−54 | Thioredoxin |
| 1288 | TNF | 1/1 | 153-280 | 59.0 | 2.6e−15 | TNF(Tumor |
| | | | | | | Necrosis |
| | | | | | | Factor) family |
| 1289 | Pyrophosphatase | 1/1 | 45-229 | 440.3 | 1e−128 | Inorganic |
| | | | | | | pyrophosphatase |
| 1290 | LIM | 1/3 | 384-442 | 56.5 | 1.5e−14 | LIM domain |
| 1290 | LIM | 2/3 | 444-501 | 58.1 | 5.2e−15 | LIM domain |
| 1290 | LIM | 3/3 | 504-570 | 37.3 | 4.2e−09 | LIM domain |
| 1292 | isodh | 1/1 | 56-379 | 301.7 | 2.7e−89 | Isocitrate/isopropylmalate |
| | | | | | | dehydrogenase |
| 1293 | cofilin_ADF | 1/1 | 19-154 | 195.2 | 1e−54 | Cofilin/tropomyosin- |
| | | | | | | type |
| | | | | | | actin-binding |
| | | | | | | pr |
| 1294 | UQ_con | 1/1 | 2-154 | 254.3 | 1.6e−72 | Ubiquitin- |
| | | | | | | conjugating |
| | | | | | | enzyme |
| 1295 | ABC_tran | 1/1 | 7-22 | 10.3 | 0.081 | ABC transporter |
| 1295 | APS_kinase | 1/1 | 5-20 | 8.3 | 0.24 | Adenylylsulfate |
| | | | | | | kinase |
| 1295 | DUF258 | 1/1 | 8-25 | 8.4 | 0.19 | Protein of |
| | | | | | | unknown |
| | | | | | | function, |
| | | | | | | DUF258 |
| 1295 | Guanylate_kin | 1/1 | 40-144 | 212.8 | 5.2e−60 | Guanylate |
| | | | | | | kinase |
| 1295 | Rad17 | 1/1 | 9-28 | 6.3 | 0.48 | Rad17 cell |
| | | | | | | cycle |
| | | | | | | checkpoint |
| | | | | | | protein |
| 1295 | SKI | 1/1 | 5-20 | 9.1 | 0.2 | Shikimate |
| | | | | | | kinase |
| 1295 | Viral_helicasel | 1/1 | 7-27 | 5.5 | 0.95 | Viral |
| | | | | | | (Superfamily 1) |
| | | | | | | RNA helicase |
| 1295 | cobW | 1/1 | 7-27 | 6.6 | 0.32 | Cobalamin |
| | | | | | | synthesis |
| | | | | | | protein/P47K |
| 1297 | Gal-bind_lectin | 1/1 | 117-247 | 215.3 | 2.8e−61 | Galactoside- |
| | | | | | | binding lectin |
| 1299 | Acetyltransf | 1/1 | 157-175 | 12.0 | 0.0055 | Acetyltransferase |
| | | | | | | (GNAT) |
| | | | | | | family |
| 1299 | PapB | 1/1 | 98-112 | 8.1 | 0.39 | Adhesin |
| | | | | | | biosynthesis |
| | | | | | | transcription |
| | | | | | | regu |
| 1303 | CH | 1/2 | 44-149 | 96.3 | 3.2e−25 | Calponin |
| | | | | | | homology (CH) |
| | | | | | | domain |
| 1303 | CH | 2/2 | 167-269 | 82.4 | 2.2e−21 | Calponin |
| | | | | | | homology (CH) |
| | | | | | | domain |
| 1303 | Filamin | 1/24 | 278-371 | 159.1 | 4.6e−47 | Filamin/ABP280 |
| | | | | | | repeat |
| 1303 | Filamin | 10/24 | 1157-1246 | 153.3 | 2.7e−45 | Filamin/ABP280 |
| | | | | | | repeat |
| 1303 | Filamin | 11/24 | 1252-1346 | 146.0 | 4.4e−43 | Filamin/ABP280 |
| | | | | | | repeat |
| 1303 | Filamin | 12/24 | 1352-1439 | 163.3 | 2.3e−48 | Filamin/ABP280 |
| | | | | | | repeat |
| 1303 | Filamin | 13/24 | 1445-1536 | 167.4 | 1.3e−49 | Filamin/ABP280 |
| | | | | | | repeat |
| 1303 | Filamin | 14/24 | 1542-1633 | 165.4 | 5.5e−49 | Filamin/ABP280 |
| | | | | | | repeat |
| 1303 | Filamin | 15/24 | 1639-1737 | 130.1 | 2.9e−38 | Filamin/ABP280 |
| | | | | | | repeat |
| 1303 | Filamin | 16/24 | 1779-1857 | 97.6 | 2.3e−28 | Filamin/ABP280 |
| | | | | | | repeat |
| 1303 | Filamin | 17/24 | 1862-1949 | 167.4 | 1.4e−49 | Filamin/ABP280 |
| | | | | | | repeat |
| 1303 | Filamin | 18/24 | 1951-2036 | 101.7 | 1.3e−29 | Filamin/ABP280 |
| | | | | | | repeat |
| 1303 | Filamin | 19/24 | 2044-2131 | 163.1 | 2.7e−48 | Filamin/ABP280 |
| | | | | | | repeat |
| 1303 | Filamin | 2/24 | 378-471 | 155.3 | 6.3e−46 | Filamin/ABP280 |
| | | | | | | repeat |
| 1303 | Filamin | 20/24 | 2132-2227 | 114.6 | 1.5e−33 | Filamin/ABP280 |
| | | | | | | repeat |
| 1303 | Filamin | 21/24 | 2235-2322 | 161.0 | 1.2e−47 | Filamin/ABP280 |
| | | | | | | repeat |
| 1303 | Filamin | 22/24 | 2329-2417 | 126.0 | 5.3e−37 | Filamin/ABP280 |
| | | | | | | repeat |
| 1303 | Filamin | 23/24 | 2426-2513 | 135.5 | 6.7e−40 | Filamin/ABP280 |
| | | | | | | repeat |
| 1303 | Filamin | 24/24 | 2554-2643 | 137.7 | 1.5e−40 | Filamin/ABP280 |
| | | | | | | repeat |
| 1303 | Filamin | 3/24 | 477-567 | 148.6 | 7e−44 | Filamin/ABP280 |
| | | | | | | repeat |
| 1303 | Filamin | 4/24 | 573-660 | 139.5 | 4.1e−41 | Filamin/ABP280 |
| | | | | | | repeat |
| 1303 | Filamin | 5/24 | 669-760 | 165.5 | 5.3e−49 | Filamin/ABP280 |
| | | | | | | repeat |
| 1303 | Filamin | 6/24 | 766-863 | 141.7 | 8.9e−42 | Filamin/ABP280 |
| | | | | | | repeat |
| 1303 | Filamin | 7/24 | 869-962 | 154.5 | 1.1e−45 | Filamin/ABP280 |
| | | | | | | repeat |
| 1303 | Filamin | 8/24 | 968-1058 | 113.1 | 4.6e−33 | Filamin/ABP280 |
| | | | | | | repeat |
| 1303 | Filamin | 9/24 | 1064-1151 | 169.7 | 2.8e−50 | Filamin/ABP280 |
| | | | | | | repeat |
| 1304 | CMD | 1/1 | 90-106 | 7.9 | 0.75 | Carboxymuconolactone |
| | | | | | | decarboxylase |
| 1305 | Ribosomal_L7Ae | 1/1 | 21-121 | 74.5 | 7.4e−21 | Ribosomal |
| | | | | | | protein |
| | | | | | | L7Ae/L30e/S12e/ |
| | | | | | | Gadd4 |
| 1306 | Dynein_light | 1/1 | 1-89 | 219.7 | 4.3e−62 | Dynein light |
| | | | | | | chain type 1 |
| 1308 | ig | 2/2 | 119-187 | 26.8 | 9.1e−06 | Immunoglobulin |
| | | | | | | domain |
| 1309 | Glypican | 1/1 | 4-578 | 1207.6 | 0 | Glypican |
| 1310 | Astacin | 1/1 | 128-321 | 395.9 | 4.8e−122 | Astacin |
| | | | | | | (Peptidase |
| | | | | | | family M12A) |
| 1310 | CUB | 1/5 | 322-431 | 204.4 | 6.2e−59 | CUB domain |
| 1310 | CUB | 2/5 | 435-544 | 228.2 | 6.1e−66 | CUB domain |
| 1310 | CUB | 3/5 | 591-700 | 210.5 | 9.9e−61 | CUB domain |
| 1310 | CUB | 4/5 | 747-856 | 206.0 | 2.2e−59 | CUB domain |
| 1310 | CUB | 5/5 | 860-973 | 173.6 | 7.4e−50 | CUB domain |
| 1310 | EGF | 1/2 | 551-587 | 33.2 | 1.1e−07 | EGF-like domain |
| 1310 | EGF | 2/2 | 707-742 | 27.2 | 5.5e−06 | EGF-like domain |
| 1310 | VSP | 1/1 | 572-589 | 5.5 | 1 | Giardia |
| | | | | | | variant- |
| | | | | | | specific |
| | | | | | | surface protein |
| 1311 | HIT | 1/1 | 14-125 | 223.0 | 4.4e−63 | HIT family |
| 1312 | proteasome | 1/1 | 56-238 | 165.7 | 3.1e−46 | Proteasome A- |
| | | | | | | type and B-type |
| 1313 | filament | 1/1 | 71-381 | 335.6 | 5.5e−97 | Intermediate |
| | | | | | | filament |
| | | | | | | protein |
| 1314 | Fasciclin | 1/4 | 134-238 | 61.9 | 1.2e−16 | Fasciclin |
| | | | | | | domain |
| 1314 | Fasciclin | 2/4 | 240-373 | 99.8 | 1.3e−27 | Fasciclin |
| | | | | | | domain |
| 1314 | Fasciclin | 3/4 | 432-500 | 10.5 | 0.078 | Fasciclin |
| | | | | | | domain |
| 1314 | Fasciclin | 4/4 | 502-634 | 235.2 | 9.5e−67 | Fasciclin |
| | | | | | | domain |
| 1315 | histone | 1/1 | 6-122 | 165.0 | 1.3e−45 | Core histone |
| | | | | | | H2A/H2B/H3/H4 |
| 1317 | ATP-grasp | 1/1 | 173-252 | 9.9 | 0.085 | ATP-grasp |
| | | | | | | domain |
| 1317 | Biotin_carb_C | 1/1 | 372-480 | 197.4 | 1.4e−56 | Biotin |
| | | | | | | carboxylase C- |
| | | | | | | terminal domain |
| 1317 | CPSase_L_D2 | 1/1 | 151-365 | 437.0 | 1.7e−127 | Carbamoyl- |
| | | | | | | phosphate |
| | | | | | | synthase L |
| | | | | | | chain, |
| 1317 | CPSase_L_chain | 1/1 | 37-149 | 205.2 | 9.8e−58 | Carbamoyl- |
| | | | | | | phosphate |
| | | | | | | synthase L |
| | | | | | | chain, |
| 1317 | HlyD | 1/1 | 633-660 | 7.1 | 0.94 | HlyD family |
| | | | | | | secretion |
| | | | | | | protein |
| 1317 | SLT_beta | 1/1 | 551-605 | 10.1 | 0.1 | Shiga-like |
| | | | | | | toxin beta |
| | | | | | | subunit |
| 1317 | biotin_lipoyl | 1/1 | 635-702 | 105.4 | 3e−28 | Biotin- |
| | | | | | | requiring |
| | | | | | | enzyme |
| 1317 | gpD | 1/1 | 169-182 | 6.2 | 0.53 | Bacteriophage |
| | | | | | | scaffolding |
| | | | | | | protein D |
| 1318 | Tropomyosin | 1/1 | 10-126 | 235.3 | 8.9e−67 | Tropomyosin |
| 1322 | Mtp | 1/1 | 36-233 | 438.5 | 5.7e−128 | Golgi 4- |
| | | | | | | transmembrane |
| | | | | | | spanning |
| | | | | | | transporter |
| 1323 | eIF5_eIF2B | 1/1 | 182-310 | 281.2 | 1.3e−80 | Domain found in |
| | | | | | | IF2B/IF5 |
| 1325 | BPL_C | 1/1 | 151-171 | 8.5 | 0.78 | Biotin protein |
| | | | | | | ligase C |
| | | | | | | terminal domain |
| 1325 | Mov34 | 1/1 | 87-196 | 149.7 | 5.1e−41 | Mov34/MPN/PAD-1 |
| | | | | | | family |
| 1326 | Ribosomal_L22 | 1/1 | 17-152 | 236.6 | 3.5e−67 | Ribosomal |
| | | | | | | protein |
| | | | | | | L22p/L17e |
| 1327 | MAPEG | 1/1 | 52-136 | 156.7 | 5.8e−45 | MAPEG family |
| 1328 | proteasome | 1/1 | 40-221 | 165.8 | 2.8e−46 | Proteasome A- |
| | | | | | | type and B-type |
| 1329 | C2 | 1/1 | 435-514 | 46.6 | 5.3e−12 | C2 domain |
| 1329 | PH | 1/1 | 314-416 | 90.8 | 3.3e−24 | PH domain |
| 1329 | RasGAP | 1/1 | 608-782 | 319.3 | 4.4e−92 | GTPase- |
| | | | | | | activator |
| | | | | | | protein for |
| | | | | | | Ras-like G |
| 1329 | SH2 | 1/2 | 20-95 | 112.0 | 1.4e−37 | SH2 domain |
| 1329 | SH2 | 2/2 | 190-265 | 116.7 | 3.6e−39 | SH2 domain |
| 1329 | SH3 | 1/1 | 121-178 | 45.0 | 9.8e−11 | SH3 domain |
| 1330 | AAA | 1/1 | 221-408 | 303.8 | 1.2e−90 | ATPase family |
| | | | | | | associated with |
| | | | | | | various |
| 1330 | IstB | 1/1 | 222-238 | 8.3 | 0.23 | IstB-like ATP |
| | | | | | | binding protein |
| 1330 | Parvo_NS1 | 1/1 | 222-240 | 7.8 | 0.17 | Parvovirus non- |
| | | | | | | structural |
| | | | | | | protein NS1 |
| 1330 | RNA_helicase | 1/1 | 222-242 | 12.8 | 0.016 | RNA helicase |
| 1331 | serpin | 1/1 | 51-415 | 283.6 | 3.8e−83 | Serpin (serine |
| | | | | | | protease |
| | | | | | | inhibitor) |
| 1332 | TGF-beta | 1/1 | 208-308 | 65.0 | 8.5e−18 | Transforming |
| | | | | | | growth factor |
| | | | | | | beta like domai |
| 1334 | BRICHOS | 1/1 | 137-231 | 132.0 | 5.2e−37 | BRICHOS domain |
| 1337 | DUF232 | 1/1 | 14-149 | 155.4 | 9.8e−43 | Putative |
| | | | | | | transcriptional |
| | | | | | | regulator |
| 1339 | Ribosomal_L27 | 1/1 | 31-101 | 71.7 | 5.3e−20 | Ribosomal L27 |
| | | | | | | protein |
| 1340 | TPR | 1/1 | 313-336 | 10.4 | 0.38 | TPR Domain |
| 1342 | sushi | 1/4 | 36-94 | 50.1 | 1.8e−11 | Sushi domain |
| | | | | | | (SCR repeat) |
| 1342 | sushi | 2/4 | 98-158 | 43.2 | 1.2e−09 | Sushi domain |
| | | | | | | (SCR repeat) |
| 1342 | sushi | 3/4 | 163-220 | 56.2 | 4.4e−13 | Sushi domain |
| | | | | | | (SCR repeat) |
| 1342 | sushi | 4/4 | 225-283 | 75.4 | 1.2e−18 | Sushi domain |
| | | | | | | (SCR repeat) |
| 1345 | UCR_hinge | 1/1 | 27-91 | 162.6 | 1.5e−46 | Ubiquinol- |
| | | | | | | cytochrome C |
| | | | | | | reductase hinge |
| | | | | | | prot |
| 1349 | AAA | 1/1 | 185-372 | 305.1 | 5e−91 | ATPase family |
| | | | | | | associated with |
| | | | | | | various |
| 1349 | IstB | 1/1 | 180-205 | 11.0 | 0.037 | IstB-like ATP |
| | | | | | | binding protein |
| 1349 | Parvo_NS1 | 1/1 | 186-207 | 15.6 | 0.00085 | Parvovirus non- |
| | | | | | | structural |
| | | | | | | protein NS1 |
| 1349 | RNA_helicase | 1/1 | 186-204 | 11.9 | 0.028 | RNA helicase |
| 1349 | Sigma54_activat | 1/1 | 186-203 | 10.4 | 0.049 | Sigma-54 |
| | | | | | | interaction |
| | | | | | | domain |
| 1351 | Robl_LC7 | 1/1 | 3-94 | 113.6 | 3.8e−30 | Roadblock/LC7 |
| | | | | | | domain |
| 1352 | YT521-B | 1/1 | 457-547 | 212.4 | 7e−60 | YT521-B-like |
| | | | | | | family |
| 1353 | AAA | 1/1 | 211-398 | 291.4 | 6.7e−87 | ATPase family |
| | | | | | | associated with |
| | | | | | | various ce |
| 1353 | RNA_helicase | 1/1 | 212-232 | 8.5 | 0.25 | RNA helicase |
| 1354 | WD40 | 1/6 | 3-41 | 19.4 | 0.001 | WD domain, G- |
| | | | | | | beta repeat |
| 1354 | WD40 | 2/6 | 47-87 | 40.1 | 1.3e−09 | WD domain, G- |
| | | | | | | beta repeat |
| 1354 | WD40 | 3/6 | 93-133 | 31.5 | 3.6e−07 | WD domain, G- |
| | | | | | | beta repeat |
| 1354 | WD40 | 4/6 | 140-195 | 42.6 | 2.5e−10 | WD domain, G- |
| | | | | | | beta repeat |
| 1354 | WD40 | 5/6 | 202-244 | 38.8 | 3.2e−09 | WD domain, G- |
| | | | | | | beta repeat |
| 1354 | WD40 | 6/6 | 253-290 | 14.7 | 0.023 | WD domain, G- |
| | | | | | | beta repeat |
| 1355 | zf-C3HC4 | 1/1 | 76-116 | 38.4 | 1.6e−10 | Zinc finger, |
| | | | | | | C3HC4 type |
| | | | | | | (RING finger) |
| 1358 | UDPGP | 1/1 | 43-462 | 892.9 | 9.5e−265 | UTP - glucose-1- |
| | | | | | | phosphate |
| | | | | | | uridylyltransferase |
| 1360 | W2 | 1/1 | 336-416 | 120.2 | 3.6e−36 | eIF4- |
| | | | | | | gamma/eIF5/eIF2- |
| | | | | | | epsilon |
| 1362 | Ribosomal_L34e | 1/1 | 5-103 | 229.8 | 4e−65 | Ribosomal |
| | | | | | | protein L34e |
| 1363 | Ribosomal_L18p | 1/1 | 26-99 | 118.5 | 1.3e−31 | Ribosomal |
| | | | | | | L18p/L5e family |
| 1365 | ATP-gua_Ptrans | 1/1 | 148-414 | 655.9 | 2.1e−193 | ATP: guanido |
| | | | | | | phosphotransferase, |
| | | | | | | C-ter |
| 1365 | ATP-gua_PtransN | 1/1 | 52-135 | 192.8 | 5.3e−54 | ATP: guanido |
| | | | | | | phosphotransferase, |
| | | | | | | N-ter |
| 1366 | DUF232 | 1/1 | 8-142 | 189.0 | 7.6e−53 | Putative |
| | | | | | | transcriptional |
| | | | | | | regulator |
| 1367 | Triabin | 1/1 | 119-136 | 12.1 | 0.017 | Triabin |
| 1367 | lipocalin | 1/1 | 38-186 | 149.1 | 3.9e−42 | Lipocalin/ |
| | | | | | | cytosolic |
| | | | | | | fatty-acid |
| | | | | | | binding pr |
| 1369 | CSD | 1/1 | 62-98 | 38.8 | 1.9e−09 | ‘Cold-shock’ |
| | | | | | | DNA-binding |
| | | | | | | domain |
| 1370 | 60s_ribosomal | 1/1 | 22-113 | 129.0 | 6.3e−37 | 60s Acidic |
| | | | | | | ribosomal |
| | | | | | | protein |
| 1371 | thiored | 1/1 | 2-106 | 155.7 | 1.2e−45 | Thioredoxin |
| 1372 | LIM | 1/2 | 28-85 | 62.3 | 3.3e−16 | LIM domain |
| 1372 | LIM | 2/2 | 137-194 | 70.7 | 1.3e−18 | LIM domain |
| 1373 | PX | 1/1 | 137-265 | 143.5 | 3.8e−39 | PX domain |
| 1373 | Sorting_nexin | 1/1 | 2-134 | 295.9 | 3.3e−91 | Sorting nexin, |
| | | | | | | N-terminal |
| | | | | | | domain |
| 1374 | rrm | 1/1 | 18-82 | 65.6 | 7.7e−18 | RNA recognition |
| | | | | | | motif. (a.k.a. |
| | | | | | | RRM, RBD, or |
| 1375 | NHL | 2/2 | 256-254 | 9.8 | 0.6 | NHL repeat |
| 1375 | Str_synth | 1/1 | 40-408 | 325.7 | 2.8e−97 | Strictosidine |
| | | | | | | synthase |
| 1377 | Cation_ATPase_C | 1/1 | 834-1012 | 238.1 | 4.2e−71 | Cation |
| | | | | | | transporting |
| | | | | | | ATPase, C- |
| | | | | | | N-terminu |
| 1377 | Cation_ATPase_N | 1/1 | 33-116 | 134.2 | 6.9e−37 | Cation |
| | | | | | | transporter/ATPase, |
| | | | | | | N-terminus |
| 1377 | E1-E2_ATPase | 1/1 | 135-366 | 391.6 | 5.1e−118 | E1-E2 ATPase |
| 1377 | Hydrolase | 1/2 | 370-633 | 31.3 | 3.4e−07 | haloacid |
| | | | | | | dehalogenase - |
| | | | | | | like hydrolase |
| 1377 | Hydrolase | 2/2 | 687-738 | 41.9 | 4.7e−10 | haloacid |
| | | | | | | dehalogenase- |
| | | | | | | like hydrolase |
| 1379 | SCAMP | 1/1 | 131-308 | 359.0 | 5e−104 | SCAMP family |
| 1380 | COX4 | 1/1 | 26-169 | 242.1 | 7.7e−69 | Cytochrome c |
| | | | | | | oxidase subunit |
| | | | | | | IV |
| 1381 | LIM | 1/1 | 260-315 | 45.6 | 1.8e−11 | LIM domain |
| 1381 | PDZ | 1/1 | 3-84 | 96.2 | 5.8e−26 | PDZ domain |
| | | | | | | (Also known as |
| | | | | | | DHR or GLGF) |
| 1382 | CoaE | 1/1 | 21-38 | 9.0 | 0.11 | Dephospho-CoA |
| | | | | | | kinase |
| 1382 | PRK | 1/1 | 22-229 | 230.6 | 2.3e−65 | Phosphoribulokinase/ |
| | | | | | | Uridine |
| | | | | | | kinase family |
| 1383 | ras | 1/1 | 61-240 | 329.8 | 7e−97 | Ras family |
| 1384 | Leptin | 1/1 | 40-185 | 392.4 | 1.4e−120 | Leptin |
| 1385 | DUF387 | 1/1 | 78-100 | 5.8 | 0.99 | Putative |
| | | | | | | transcriptional |
| | | | | | | regulators (Yp |
| 1385 | Ribosomal_S25 | 1/1 | 1-113 | 262.3 | 6.5e−75 | S25 ribosomal |
| | | | | | | protein |
| 1386 | DEAD | 1/1 | 51-254 | 242.3 | 1.5e−73 | DEAD/DEAH box |
| | | | | | | helicase |
| 1386 | helicase_C | 1/1 | 301-372 | 127.9 | 6.6e−35 | Helicase |
| | | | | | | conserved C- |
| | | | | | | terminal domain |
| 1388 | ank | 2/5 | 110-142 | 32.6 | 1.8e−07 | Ankyrin repeat |
| 1388 | ank | 3/5 | 143-175 | 16.5 | 0.0069 | Ankyrin repeat |
| 1388 | ank | 4/5 | 182-214 | 39.5 | 1.9e−09 | Ankyrin repeat |
| 1388 | ank | 5/5 | 216-248 | 41.0 | 7.1e−10 | Ankyrin repeat |
| 1390 | PX | 1/1 | 505-626 | 95.6 | 9.9e−25 | PX domain |
| 1390 | PXA | 1/1 | 122-253 | 42.5 | 2.7e−10 | PXA domain |
| 1391 | SUI1 | 1/1 | 9-109 | 225.9 | 6e−64 | Translation |
| | | | | | | initiation |
| | | | | | | factor SUI1 |
| 1392 | perilipin | 1/1 | 2-396 | 813.3 | 6.8e−244 | Perilipin |
| | | | | | | family |
| 1393 | PRA1 | 1/1 | 8-55 | 15.3 | 0.0047 | Prenylated rab |
| | | | | | | acceptor (PRA1) |
| 1395 | ENV_polyprotein | 1/1 | 265-486 | 159.2 | 1.6e−46 | ENV polyprotein |
| | | | | | | (coat |
| | | | | | | polyprotein) |
| 1396 | LRR | 1/10 | 82-105 | 16.8 | 0.007 | Leucine Rich |
| | | | | | | Repeat |
| 1396 | LRR | 2/10 | 106-129 | 12.9 | 0.085 | Leucine Rich |
| | | | | | | Repeat |
| 1396 | LRR | 3/10 | 130-149 | 14.8 | 0.025 | Leucine Rich |
| | | | | | | Repeat |
| 1396 | LRR | 4/10 | 151-174 | 9.1 | 1 | Leucine Rich |
| | | | | | | Repeat |
| 1396 | LRR | 7/10 | 222-245 | 17.1 | 0.0055 | Leucine Rich |
| | | | | | | Repeat |
| 1396 | LRR | 8/10 | 246-269 | 9.8 | 0.65 | Leucine Rich |
| | | | | | | Repeat |
| 1396 | LRR | 9/10 | 270-292 | 15.3 | 0.018 | Leucine Rich |
| | | | | | | Repeat |
| 1396 | LRRNT | 1/2 | 53-80 | 43.3 | 2.8e−10 | Leucine rich |
| | | | | | | repeat N- |
| | | | | | | terminal domain |
| 1397 | Ribosomal_S27 | 1/1 | 101-147 | 119.0 | 8.7e−32 | Ribosomal |
| | | | | | | protein S27a |
| 1397 | ubiquitin | 1/1 | 1-74 | 153.1 | 4.8e−42 | Ubiquitin |
| | | | | | | family |
| 1398 | Kunitz_BPTI | 1/3 | 36-86 | 93.6 | 2.6e−29 | Kunitz/Bovine |
| | | | | | | pancreatic |
| | | | | | | trypsin |
| | | | | | | inhibito |
| 1398 | Kunitz_BPTI | 2/3 | 96-149 | 38.7 | 2.1e−11 | Kunitz/Bovine |
| | | | | | | pancreatic |
| | | | | | | trypsin |
| | | | | | | inhibito |
| 1398 | Kunitz_BPTI | 3/3 | 158-208 | 85.3 | 1.3e−26 | Kunitz/Bovine |
| | | | | | | pancreatic |
| | | | | | | trypsin |
| | | | | | | inhibito |
| 1398 | kinesin | 1/1 | 221-231 | 5.8 | 0.8 | Kinesin motor |
| | | | | | | domain |
| 1399 | proteasome | 1/1 | 28-216 | 186.1 | 4.6e−52 | Proteasome A- |
| | | | | | | type and B-type |
| 1401 | AAA | 1/1 | 169-356 | 316.0 | 2.7e−94 | ATPase family |
| | | | | | | associated with |
| | | | | | | various |
| 1401 | IstB | 1/1 | 170-186 | 7.9 | 0.31 | IstB-like ATP |
| | | | | | | binding protein |
| 1401 | Parvo_NS1 | 1/1 | 171-188 | 6.5 | 0.4 | Parvovirus non- |
| | | | | | | structural |
| | | | | | | protein NS1 |
| 1401 | RNA_helicase | 1/1 | 170-191 | 11.7 | 0.032 | RNA helicase |
| 1401 | Sigma54_activat | 1/1 | 171-187 | 7.1 | 0.44 | Sigma-54 |
| | | | | | | interaction |
| | | | | | | domain |
| 1403 | TB | 1/1 | 46-71 | 8.6 | 0.35 | TB domain |
| 1403 | kazal | 1/2 | 121-167 | 48.1 | 1.4e−10 | Kazal-type |
| | | | | | | serine protease |
| | | | | | | inhibitor |
| | | | | | | domain |
| 1403 | kazal | 2/2 | 195-243 | 36.5 | 1.4e−07 | Kazal-type |
| | | | | | | serine protease |
| | | | | | | inhibitor |
| | | | | | | domain |
| 1404 | 60s_ribosomal | 1/1 | 17-114 | 114.8 | 1e−32 | 60s Acidic |
| | | | | | | ribosomal |
| | | | | | | protein |
| 1404 | Peptidase_M4_C | 1/1 | 12-32 | 6.5 | 0.31 | Thermolysin |
| | | | | | | metallopeptidase, |
| | | | | | | alpha-he |
| 1406 | zf-C3HC4 | 1/1 | 79-102 | 10.3 | 0.069 | Zinc finger, |
| | | | | | | C3HC4 type |
| | | | | | | (RING finger) |
| 1408 | Adap_comp_sub | 1/1 | 58-95 | 7.6 | 0.054 | Adaptor |
| | | | | | | complexes |
| | | | | | | medium subunit |
| | | | | | | famil |
| 1408 | Clat_adaptor_s | 1/1 | 1-142 | 325.8 | 4.8e−94 | Clathrin |
| | | | | | | adaptor complex |
| | | | | | | small chain |
| 1409 | Calsequestrin | 2/2 | 235-348 | 20.2 | 1.6e−05 | Calsequestrin |
| 1409 | DSBA | 1/3 | 83-104 | 10.2 | 0.1 | DSBA |
| | | | | | | oxidoreductase |
| 1409 | DSBA | 2/3 | 194-225 | 7.4 | 0.62 | DSBA |
| | | | | | | oxidoreductase |
| 1409 | DSBA | 3/3 | 547-576 | 16.2 | 0.0021 | DSBA |
| | | | | | | oxidoreductase |
| 1409 | thiored | 1/3 | 61-169 | 184.7 | 2.2e−54 | Thioredoxin |
| 1409 | thiored | 2/3 | 176-284 | 193.4 | 5.2e−57 | Thioredoxin |
| 1409 | thiored | 3/3 | 524-636 | 182.5 | 1e−53 | Thioredoxin |
| 1410 | APS_kinase | 1/1 | 21-39 | 10.8 | 0.042 | Adenylylsulfate |
| | | | | | | kinase |
| 1410 | CoaE | 1/1 | 20-198 | 321.3 | 5e−96 | Dephospho-CoA |
| | | | | | | kinase |
| 1410 | PRK | 1/1 | 21-39 | 18.5 | 0.00032 | Phosphoribulokinase/ |
| | | | | | | Uridine |
| | | | | | | kinase |
| 1410 | UPF0042 | 1/1 | 19-48 | 7.4 | 0.33 | Uncharacterised |
| | | | | | | P-loop ATPase |
| | | | | | | protein |
| 1411 | Fz | 1/1 | 1-21 | 22.8 | 7.9e−07 | Fz domain |
| 1411 | Zn_carbOpept | 1/2 | 50-160 | 69.8 | 6.6e−18 | Zinc |
| | | | | | | carboxypeptidase |
| 1411 | Zn_carbOpept | 2/2 | 172-431 | 90.9 | 1.3e−23 | Zinc |
| | | | | | | carboxypeptidase |
| 1412 | ANATO | 1/3 | 36-69 | 41.2 | 5.9e−15 | Anaphylotoxin- |
| | | | | | | like domain |
| 1412 | ANATO | 2/3 | 77-110 | 29.6 | 1.4e−10 | Anaphylotoxin- |
| | | | | | | like domain |
| 1412 | ANATO | 3/3 | 112-144 | 37.0 | 2.3e−13 | Anaphylotoxin- |
| | | | | | | like domain |
| 1412 | EGF | 1/8 | 180-214 | 16.3 | 0.0067 | EGF-like domain |
| 1412 | EGF | 3/8 | 312-354 | 19.9 | 0.00063 | EGF-like domain |
| 1412 | EGF | 4/8 | 360-397 | 40.6 | 8.8e−10 | EGF-like domain |
| 1412 | EGF | 5/8 | 403-439 | 25.9 | 1.3e−05 | EGF-like domain |
| 1412 | EGF | 6/8 | 445-479 | 13.2 | 0.05 | EGF-like domain |
| 1412 | EGF | 7/8 | 485-523 | 29.3 | 1.4e−06 | EGF-like domain |
| 1413 | rrm | 1/3 | 192-233 | 32.9 | 3.1e−08 | RNA recognition |
| | | | | | | motif. (a.k.a. |
| | | | | | | RRM, RBD, or |
| 1413 | rrm | 2/3 | 260-327 | 66.4 | 4.4e−18 | RNA recognition |
| | | | | | | motif. (a.k.a. |
| | | | | | | RRM, RBD, or |
| 1413 | rrm | 3/3 | 346-414 | 75.0 | 1.3e−20 | RNA recognition |
| | | | | | | motif. (a.k.a. |
| | | | | | | RRM, RBD, or |
| 1414 | Ribosomal_S11 | 1/1 | 29-147 | 234.9 | 3.4e−72 | Ribosomal |
| | | | | | | protein S11 |
| 1415 | Ribosomal_L29e | 1/1 | 25-64 | 83.6 | 4.4e−23 | Ribosomal L29e |
| | | | | | | protein family |
| 1416 | Ribosomal_L1 | 1/1 | 5-213 | 348.7 | 6.4e−127 | Ribosomal |
| | | | | | | protein |
| | | | | | | L1p/L10e family |
| 1418 | LRR | 1/10 | 67-90 | 15.2 | 0.019 | Leucine Rich |
| | | | | | | Repeat |
| 1418 | LRR | 10/10 | 305-330 | 13.9 | 0.044 | Leucine Rich |
| | | | | | | Repeat |
| 1418 | LRR | 2/10 | 91-116 | 11.0 | 0.3 | Leucine Rich |
| | | | | | | Repeat |
| 1418 | LRR | 4/10 | 138-159 | 16.1 | 0.011 | Leucine Rich |
| | | | | | | Repeat |
| 1418 | LRR | 5/10 | 160-184 | 12.9 | 0.088 | Leucine Rich |
| | | | | | | Repeat |
| 1418 | LRR | 6/10 | 185-204 | 16.6 | 0.0078 | Leucine Rich |
| | | | | | | Repeat |
| 1418 | LRR | 7/10 | 206-229 | 17.1 | 0.0057 | Leucine Rich |
| | | | | | | Repeat |
| 1418 | LRR | 8/10 | 230-253 | 9.7 | 0.71 | Leucine Rich |
| | | | | | | Repeat |
| 1418 | LRR | 9/10 | 255-277 | 12.7 | 0.096 | Leucine Rich |
| | | | | | | Repeat |
| 1418 | LRRNT | 1/1 | 36-66 | 40.5 | 1.7e−09 | Leucine rich |
| | | | | | | repeat N- |
| | | | | | | terminal domain |
| 1419 | AhpC-TSA | 1/1 | 81-230 | 247.7 | 1.6e−70 | AhpC/TSA family |
| 1419 | Syndecan | 1/1 | 18-49 | 6.4 | 0.062 | Syndecan domain |
| 1421 | Triabin | 1/1 | 120-135 | 17.7 | 0.00038 | Triabin |
| 1421 | lipocalin | 1/1 | 41-185 | 111.9 | 2.6e−31 | Lipocalin/ |
| | | | | | | cytosolic |
| | | | | | | fatty-acid |
| | | | | | | binding pr |
| 1422 | Ribosomal_L21e | 1/1 | 2-100 | 241.6 | 1.1e−68 | Ribosomal |
| | | | | | | protein L21e |
| 1423 | Ribosomal_S10 | 1/1 | 19-114 | 193.7 | 2.8e−54 | Ribosomal |
| | | | | | | protein |
| | | | | | | S10p/S20e |
| 1424 | Ribosomal_L23 | 1/1 | 74-153 | 112.4 | 5e−32 | Ribosomal |
| | | | | | | protein L23 |
| 1424 | Ribosomal_L23eN | 1/1 | 15-68 | 63.2 | 1.3e−16 | Ribosomal |
| | | | | | | protein L23, N- |
| | | | | | | terminal dom |
| 1425 | DUF435 | 1/1 | 98-383 | 445.3 | 5.3e−130 | Membrane |
| | | | | | | protein of |
| | | | | | | unknown |
| | | | | | | function (D |
| 1427 | FeoB | 1/1 | 247-262 | 12.0 | 0.0065 | Ferrous iron |
| | | | | | | transport |
| | | | | | | protein B |
| 1427 | MMR_HSR1 | 1/1 | 118-393 | 172.0 | 9.9e−48 | GTPase of |
| | | | | | | unknown |
| | | | | | | function |
| 1429 | Peptidase_C13 | 1/1 | 6-327 | 698.1 | 4.2e−206 | Peptidase C13 |
| | | | | | | family |
| 1430 | ER_lumen_recept | 1/1 | 1-202 | 527.3 | 1.1e−154 | ER lumen |
| | | | | | | protein |
| | | | | | | retaining |
| | | | | | | receptor |
| 1431 | COLFI | 1/1 | 1149-1365 | 538.4 | 6.8e−210 | Fibrillar |
| | | | | | | collagen C- |
| | | | | | | terminal domain |
| 1431 | Collagen | 1/19 | 33-72 | 26.5 | 8.5e−06 | Collagen triple |
| | | | | | | helix repeat |
| | | | | | | (20 copies) |
| 1431 | Collagen | 10/19 | 529-567 | 30.1 | 8.6e−07 | Collagen triple |
| | | | | | | helix repeat |
| | | | | | | (20 copies) |
| 1431 | Collagen | 11/19 | 568-627 | 47.1 | 1.9e−11 | Collagen triple |
| | | | | | | helix repeat |
| | | | | | | (20 copies) |
| 1431 | Collagen | 12/19 | 628-687 | 49.2 | 4.9e−12 | Collagen triple |
| | | | | | | helix repeat |
| | | | | | | (20 copies) |
| 1431 | Collagen | 13/19 | 691-750 | 61.7 | 1.8e−15 | Collagen triple |
| | | | | | | helix repeat |
| | | | | | | (20 copies) |
| 1431 | Collagen | 14/19 | 751-810 | 45.7 | 4.5e−11 | Collagen triple |
| | | | | | | helix repeat |
| | | | | | | (20 copies) |
| 1431 | Collagen | 15/19 | 811-870 | 42.8 | 2.7e−10 | Collagen triple |
| | | | | | | helix repeat |
| | | | | | | (20 copies) |
| 1431 | Collagen | 16/19 | 874-933 | 60.0 | 5.3e−15 | Collagen triple |
| | | | | | | helix repeat |
| | | | | | | (20 copies) |
| 1431 | Collagen | 17/19 | 934-990 | 41.1 | 8.1e−10 | Collagen triple |
| | | | | | | helix repeat |
| | | | | | | (20 copies) |
| 1431 | Collagen | 18/19 | 991-1050 | 51.5 | 1.1e−12 | Collagen triple |
| | | | | | | helix repeat |
| | | | | | | (20 copies) |
| 1431 | Collagen | 19/19 | 1051-1105 | 40.4 | 1.3e−09 | Collagen triple |
| | | | | | | helix repeat |
| | | | | | | (20 copies) |
| 1431 | Collagen | 2/19 | 90-147 | 42.6 | 3.1e−10 | Collagen triple |
| | | | | | | helix repeat |
| | | | | | | (20 copies) |
| 1431 | Collagen | 3/19 | 148-207 | 59.9 | 5.6e−15 | Collagen triple |
| | | | | | | helix repeat |
| | | | | | | (20 copies) |
| 1431 | Collagen | 4/19 | 208-267 | 48.5 | 7.5e−12 | Collagen triple |
| | | | | | | helix repeat |
| | | | | | | (20 copies) |
| 1431 | Collagen | 5/19 | 268-327 | 52.4 | 6.6e−13 | Collagen triple |
| | | | | | | helix repeat |
| | | | | | | (20 copies) |
| 1431 | Collagen | 6/19 | 328-387 | 54.7 | 1.5e−13 | Collagen triple |
| | | | | | | helix repeat |
| | | | | | | (20 copies) |
| 1431 | Collagen | 7/19 | 388-415 | 19.4 | 0.00075 | Collagen triple |
| | | | | | | helix repeat |
| | | | | | | (20 copies) |
| 1431 | Collagen | 8/19 | 418-465 | 40.7 | 1.1e−09 | Collagen triple |
| | | | | | | helix repeat |
| | | | | | | (20 copies) |
| 1431 | Collagen | 9/19 | 469-528 | 55.6 | 8.3e−14 | Collagen triple |
| | | | | | | helix repeat |
| | | | | | | (20 copies) |
| 1432 | DJ-1_PfpI | 1/1 | 4-173 | 203.7 | 1.7e−57 | DJ-1/PfpI |
| | | | | | | family |
| 1433 | Gastrin | 1/1 | 2-115 | 198.2 | 1.3e−55 | Gastrin/cholecy |
| | | | | | | stokinin family |
| 1435 | LRR | 1/7 | 41-63 | 14.2 | 0.038 | Leucine Rich |
| | | | | | | Repeat |
| 1435 | LRR | 2/7 | 64-86 | 13.8 | 0.049 | Leucine Rich |
| | | | | | | Repeat |
| 1435 | LRR | 3/7 | 87-109 | 11.7 | 0.19 | Leucine Rich |
| | | | | | | Repeat |
| 1435 | LRR | 4/7 | 110-134 | 13.6 | 0.055 | Leucine Rich |
| | | | | | | Repeat |
| 1435 | LRR | 5/7 | 135-157 | 15.6 | 0.015 | Leucine Rich |
| | | | | | | Repeat |
| 1435 | LRR | 6/7 | 158-180 | 13.4 | 0.061 | Leucine Rich |
| | | | | | | Repeat |
| 1435 | LRR | 7/7 | 181-203 | 17.7 | 0.0039 | Leucine Rich |
| | | | | | | Repeat |
| 1436 | DUF435 | 1/1 | 62-348 | 516.0 | 2.7e−151 | Membrane |
| | | | | | | protein of |
| | | | | | | unknown |
| | | | | | | function |
| | | | | | | (DUF435 |
| 1437 | asp | 1/1 | 39-427 | 645.9 | 2.2e−190 | Eukaryotic |
| | | | | | | aspartyl |
| | | | | | | protease |
| 1438 | Clq | 1/1 | 123-247 | 251.9 | 8.8e−72 | Clq domain |
| 1438 | Collagen | 1/2 | 33-68 | 27.2 | 5.4e−06 | Collagen triple |
| | | | | | | helix repeat |
| | | | | | | (20 copies) |
| 1438 | Collagen | 2/2 | 69-114 | 24.2 | 3.5e−05 | Collagen triple |
| | | | | | | helix repeat |
| | | | | | | (20 copies) |
| 1439 | ras | 1/1 | 7-193 | 319.3 | 9.6e−94 | Ras family |
| 1440 | Rho_GDI | 1/1 | 1-201 | 502.6 | 3e−147 | RHO protein GDP |
| | | | | | | dissociation |
| | | | | | | inhibitor |
| 1441 | Clathrin_lg_ch | 1/2 | 3-162 | 294.2 | 1.7e−84 | Clathrin light |
| | | | | | | chain |
| 1441 | Clathrin_lg_ch | 2/2 | 163-217 | 124.0 | 2.7e−33 | Clathrin light |
| | | | | | | chain |
| 1441 | Myc-LZ | 1/1 | 98-130 | 8.4 | 0.85 | Myc leucine |
| | | | | | | zipper domain |
| 1443 | HMG_box | 1/2 | 6-79 | 63.4 | 3.4e−16 | HMG (high |
| | | | | | | mobility group) |
| | | | | | | box |
| 1443 | HMG_box | 2/2 | 95-163 | 129.7 | 5.5e−35 | HMG (high |
| | | | | | | mobility group) |
| | | | | | | box |
| 1444 | IBN_NT | 1/1 | 31-97 | 24.8 | 2.7e−05 | Importin-beta |
| | | | | | | N-terminal |
| | | | | | | domain |
| 1445 | EGF | 1/1 | 146−181 | 23.8 | 5.2e−05 | EGF-like domain |
| 1447 | Spermine_synth | 1/1 | 109-366 | 384.7 | 7e−116 | Spermine/spermidine |
| | | | | | | synthase |
| 1449 | ig | 1/1 | 34-116 | 14.2 | 0.029 | Immunoglobulin |
| | | | | | | domain |
| 1451 | Ribosomal_S6 | 1/1 | 3-96 | 17.8 | 0.00053 | Ribosomal |
| | | | | | | protein S6 |
| 1453 | Ribosomal_S17 | 1/1 | 75-145 | 149.1 | 4e−43 | Ribosomal |
| | | | | | | protein S17 |
| 1454 | DUF196 | 1/1 | 87-106 | 7.3 | 0.84 | Uncharacterized |
| | | | | | | ACR, COG1343 |
| 1454 | ras | 1/1 | 13-206 | 381.5 | 3e−112 | Ras family |
| 1455 | Folate_rec | 1/1 | 6-249 | 561.2 | 7e−165 | Folate receptor |
| | | | | | | family |
| 1458 | serpin | 1/1 | 139-498 | 455.7 | 4.5e−134 | Serpin (serine |
| | | | | | | protease |
| | | | | | | inhibitor) |
| 1459 | cytochrome_b_C | 1/1 | 258-359 | 152.3 | 8.2e−42 | Cytochrome b(C- |
| | | | | | | terminal)/b6/pe |
| | | | | | | tD |
| 1459 | cytochrome_b_N | 1/1 | 1-204 | 376.5 | 1e−115 | Cytochrome b(N- |
| | | | | | | terminal)/b6/pe |
| | | | | | | tB |
| 1460 | Ribosomal_L32e | 1/1 | 16-125 | 267.6 | 1.4e−83 | Ribosomal |
| | | | | | | protein L32 |
| 1462 | ECH | 1/1 | 68-248 | 150.0 | 2.2e−43 | Enoyl-CoA |
| | | | | | | hydratase/isomerase |
| | | | | | | family |
| 1463 | filament | 1/1 | 83-394 | 595.8 | 2.5e−175 | Intermediate |
| | | | | | | filament |
| | | | | | | protein |
| 1464 | DEAD | 1/2 | 15-62 | 52.1 | 4.7e−15 | DEAD/DEAH box |
| | | | | | | helicase |
| 1464 | DEAD | 2/2 | 281-444 | 93.8 | 7.2e−28 | DEAD/DEAH box |
| | | | | | | helicase |
| 1464 | SPRY | 1/1 | 130-246 | 121.0 | 2e−32 | SPRY domain |
| 1464 | helicase_C | 1/1 | 539-610 | 108.3 | 2.6e−29 | Helicase |
| | | | | | | conserved C- |
| | | | | | | terminal domain |
| 1465 | IGFBP | 1/1 | 37-94 | 13.4 | 0.017 | Insulin-like |
| | | | | | | growth factor |
| | | | | | | binding protein |
| 1465 | PDZ | 1/1 | 370-465 | 50.8 | 5.4e−13 | PDZ domain |
| | | | | | | (Also known as |
| | | | | | | DHR or GLGF) |
| 1465 | kazal | 1/1 | 109-155 | 29.4 | 8.9e−06 | Kazal-type |
| | | | | | | serine protease |
| | | | | | | inhibitor |
| | | | | | | domain |
| 1465 | trypsin | 1/1 | 205-364 | 74.5 | 7.7e−23 | Trypsin |
| 1466 | zf-CCHC | 1/7 | 4-21 | 32.6 | 3.4e−07 | Zinc knuckle |
| 1466 | zf-CCHC | 2/7 | 52-69 | 27.6 | 8.5e−06 | Zinc knuckle |
| 1466 | Zf-CCHC | 3/7 | 72-89 | 30.5 | 1.3e−06 | Zinc knuckle |
| 1466 | zf-CCHC | 4/7 | 96-113 | 31.8 | 5.4e−07 | Zinc knuckle |
| 1466 | zf-CCHC | 5/7 | 117-134 | 30.8 | 1.1e−06 | Zinc knuckle |
| 1466 | zf-CCHC | 6/7 | 135-152 | 28.2 | 5.8e−06 | Zinc knuckle |
| 1466 | zf-CCHC | 7/7 | 156-173 | 31.3 | 7.9e−07 | Zinc knuckle |
| 1467 | TIMP | 1/1 | 22-194 | 472.6 | 3.7e−172 | Tissue |
| | | | | | | inhibitor of |
| | | | | | | metalloproteinase |
| 1468 | efhand | 1/5 | 27-55 | 21.1 | 0.00061 | EF hand |
| 1468 | efhand | 3/5 | 94-122 | 31.9 | 6.3e−07 | EF hand |
| 1468 | efhand | 4/5 | 130-158 | 10.4 | 0.54 | EF hand |
| 1468 | efhand | 5/5 | 160-176 | 9.8 | 0.82 | EF hand |
| 1469 | KE2 | 1/1 | 23-130 | 128.8 | 1e−34 | KE2 family |
| | | | | | | protein |
| 1470 | Ribosomal_L11 | 1/1 | 161-229 | 106.5 | 5.2e−28 | Ribosomal |
| | | | | | | protein L11, |
| | | | | | | RNA binding do |
| 1470 | Ribosomal_L11_N | 1/1 | 98-156 | 93.4 | 1.1e−25 | Ribosomal |
| | | | | | | protein L11, N- |
| | | | | | | terminal dom |
| 1471 | Mov34 | 1/1 | 4-118 | 168.8 | 9.3e−47 | Mov34/MPN/PAD-1 |
| | | | | | | family |
| 1472 | Na_K-ATPase | 1/1 | 1-274 | 477.9 | 7.9e−140 | Sodium/ |
| | | | | | | potassium |
| | | | | | | ATPase beta |
| | | | | | | chain |
| 1475 | hexapep | 1/3 | 26-43 | 13.4 | 0.11 | Bacterial |
| | | | | | | transferase |
| | | | | | | hexapeptide |
| | | | | | | (four rep |
| 1475 | hexapep | 3/3 | 110-127 | 12.8 | 0.16 | Bacterial |
| | | | | | | transferase |
| | | | | | | hexapeptide |
| | | | | | | (four rep |
| 1476 | tubulin | 1/1 | 45-244 | 374.8 | 1.4e−109 | Tubulin/FtsZ |
| | | | | | | family, GTPase |
| | | | | | | domain |
| 1476 | tubulin_C | 1/1 | 246-383 | 270.9 | 1.7e−77 | Tubulin/FtsZ |
| | | | | | | family, C- |
| | | | | | | terminal domain |
| 1477 | Calpain_inhib | 1/5 | 53-183 | 179.0 | 1.5e−51 | Calpain |
| | | | | | | inhibitor |
| 1477 | Calpain_inhib | 2/5 | 186-317 | 197.8 | 4.3e−57 | Calpain |
| | | | | | | inhibitor |
| 1477 | Calpain_inhib | 3/5 | 323-460 | 221.0 | 6.8e−64 | Calpain |
| | | | | | | inhibitor |
| 1477 | Calpain_inhib | 4/5 | 466-597 | 233.3 | 1.6e−67 | Calpain |
| | | | | | | inhibitor |
| 1478 | DAD | 1/1 | 1-113 | 258.5 | 5.9e−88 | DAD family |
| 1479 | PSI | 1/1 | 514-542 | 13.0 | 0.0067 | Plexin repeat |
| 1479 | Sema | 1/1 | 55-496 | 841.8 | 2.3e−249 | Sema domain |
| 1479 | ig | 1/1 | 584-645 | 22.3 | 0.00017 | Immunoglobulin |
| | | | | | | domain |
| 1480 | Glyco_hydro_2 | 1/1 | 226-327 | 140.8 | 1.8e−43 | Glycosyl |
| | | | | | | hydrolases |
| | | | | | | family 2, |
| | | | | | | immunog |
| 1480 | Glyco_hydro_2_C | 1/1 | 329-631 | 597.7 | 6.8e−176 | Glycosyl |
| | | | | | | hydrolases |
| | | | | | | family 2, TIM |
| | | | | | | bar |
| 1480 | Glyco_hydro_2_N | 1/1 | 22-224 | 379.7 | 3e−110 | Glycosyl |
| | | | | | | hydrolases |
| | | | | | | family 2, sugar b |
| 1481 | F-box | 1/1 | 232-279 | 22.4 | 0.00033 | F-box domain |
| 1481 | WD40 | 1/5 | 359-395 | 17.5 | 0.0035 | WD domain, G- |
| | | | | | | beta repeat |
| 1481 | WD40 | 2/5 | 398-434 | 29.1 | 1.7e−06 | WD domain, G- |
| | | | | | | beta repeat |
| 1483 | ras | 1/1 | 12-169 | 221.6 | 9.4e−65 | Ras family |
| 1484 | MHC_II_beta | 1/1 | 40-116 | 164.6 | 1.7e−45 | Class II |
| | | | | | | histocompatibility |
| | | | | | | antigen, |
| 1484 | ig | 1/1 | 136-201 | 40.6 | 1.4e−09 | Immunoglobulin |
| | | | | | | domain |
| 1486 | Gal-bind_lectin | 1/1 | 5-137 | 105.1 | 2.8e−29 | Galactoside- |
| | | | | | | binding lectin |
| 1487 | NAP | 1/1 | 75-348 | 644.5 | 5.8e−190 | Nucleosome |
| | | | | | | assembly |
| | | | | | | protein (NAP) |
| 1488 | serpin | 1/1 | 24-398 | 542.0 | 1.3e−159 | Serpin (serine |
| | | | | | | protease |
| | | | | | | inhibitor) |
| 1489 | SRCR | 1/1 | 27-124 | 129.9 | 2.4e−35 | Scavenger |
| | | | | | | receptor |
| | | | | | | cysteine-rich |
| | | | | | | domain |
| 1490 | Ribosomal_L3 | 1/1 | 50-348 | 664.8 | 4.5e−196 | Ribosomal |
| | | | | | | protein L3 |
| 1491 | tubulin | 1/1 | 49-246 | 376.0 | 6.1e−110 | Tubulin/FtsZ |
| | | | | | | family, GTPase |
| | | | | | | domain |
| 1491 | tubulin_C | 1/1 | 248-393 | 270.6 | 2e−77 | Tubulin/FtsZ |
| | | | | | | family, C- |
| | | | | | | terminal domain |
| 1493 | efhand | 1/2 | 127-155 | 27.6 | 9.5e−06 | EF hand |
| 1493 | efhand | 2/2 | 163-191 | 22.3 | 0.00029 | EF hand |
| 1494 | HEAT | 1/15 | 6-42 | 14.2 | 0.015 | HEAT repeat |
| 1494 | HEAT | 10/15 | 357-395 | 39.8 | 3.4e−10 | HEAT repeat |
| 1494 | HEAT | 11/15 | 396-434 | 32.3 | 6e−08 | HEAT repeat |
| 1494 | HEAT | 12/15 | 435-473 | 25.9 | 4.9e−06 | HEAT repeat |
| 1494 | HEAT | 13/15 | 474-512 | 30.3 | 2.3e−07 | HEAT repeat |
| 1494 | HEAT | 14/15 | 513-551 | 50.2 | 2.6e−13 | HEAT repeat |
| 1494 | HEAT | 2/15 | 43-80 | 24.1 | 1.6e−05 | HEAT repeat |
| 1494 | HEAT | 3/15 | 82-119 | 26.3 | 3.8e−06 | HEAT repeat |
| 1494 | HEAT | 4/15 | 120-157 | 23.0 | 3.5e−05 | HEAT repeat |
| 1494 | HEAT | 5/15 | 158-196 | 44.6 | 1.3e−11 | HEAT repeat |
| 1494 | HEAT | 6/15 | 197-235 | 40.9 | 1.7e−10 | HEAT repeat |
| 1494 | HEAT | 7/15 | 236-274 | 33.5 | 2.6e−08 | HEAT repeat |
| 1494 | HEAT | 8/15 | 275-313 | 47.4 | 1.9e−12 | HEAT repeat |
| 1494 | HEAT | 9/15 | 318-356 | 28.7 | 6.9e−07 | HEAT repeat |
| 1495 | ATP1G1_PLM_MAT8 | 1/1 | 133-177 | 70.5 | 1.7e−20 | ATP1G1/PLM/MAT8 |
| | | | | | | family |
| 1496 | PMP22_Claudin | 1/1 | 1-160 | 297.8 | 1.3e−85 | PMP- |
| | | | | | | 22/EMP/MP20/Claudin |
| | | | | | | family |
| 1497 | BAH | 1/2 | 755-880 | 142.7 | 6.4e−39 | BAH domain |
| 1497 | BAH | 2/2 | 966-1100 | 137.6 | 2.2e−37 | BAH domain |
| 1497 | DNA_methylase | 1/3 | 1139-1230 | 54.6 | 2.8e−14 | C-5 cytosine- |
| | | | | | | specific DNA |
| | | | | | | methylase |
| 1497 | DNA_methylase | 2/3 | 1258-1317 | 31.7 | 6.7e−08 | C-5 cytosine- |
| | | | | | | specific DNA |
| | | | | | | methylase |
| 1497 | DNA_methylase | 3/3 | 1519-1594 | 69.5 | 2.1e−18 | C-5 cytosine- |
| | | | | | | specific DNA |
| | | | | | | methylase |
| 1497 | zf-CXXC | 1/1 | 645-691 | 87.4 | 6.3e−36 | CXXC zinc |
| | | | | | | finger |
| 1498 | Adrenomedullin | 1/1 | 21-185 | 433.5 | 1.9e−126 | Adrenomedullin |
| 1499 | S_100 | 1/1 | 5-45 | 60.9 | 1.1e−15 | S-100/ICaBP |
| | | | | | | type calcium |
| | | | | | | binding domain |
| 1499 | efhand | 2/2 | 51-79 | 11.2 | 0.34 | EF hand |
| 1500 | PMP22_Claudin | 1/1 | 3-180 | 162.7 | 6.9e−46 | PMP- |
| | | | | | | 22/EMP/MP20/Claudin |
| | | | | | | family |
| 1501 | PNPase | 1/1 | 18-28 | 7.2 | 0.065 | Polyribonucleotide |
| | | | | | | nucleotidyltransferase, |
| 1501 | RNase_PH | 1/1 | 31-166 | 125.2 | 9.5e−35 | 3′ |
| | | | | | | exoribonuclease |
| | | | | | | family, domain 1 |
| 1501 | RNase_PH_C | 1/1 | 191-258 | 42.4 | 1.1e−10 | 3′ |
| | | | | | | exoribonuclease |
| | | | | | | family, domain 2 |
| 1502 | WD40 | 1/4 | 42-80 | 35.1 | 3.6e−08 | WD domain, G- |
| | | | | | | beta repeat |
| 1502 | WD40 | 2/4 | 132-170 | 24.2 | 4.4e−05 | WD domain, G- |
| | | | | | | beta repeat |
| 1503 | homeobox | 1/1 | 19-69 | 14.4 | 0.085 | Homeobox domain |
| 1504 | Fe-ADH | 1/2 | 4-205 | 121.3 | 4.4e−34 | Iron-containing |
| | | | | | | alcohol |
| | | | | | | dehydrogenase |
| 1504 | Fe-ADH | 2/2 | 228-290 | 64.2 | 1.3e−17 | Iron-containing |
| | | | | | | alcohol |
| | | | | | | dehydrogenase |
| 1505 | Ribosomal_L37ae | 1/1 | 2-92 | 223.3 | 3.5e−63 | Ribosomal L37ae |
| | | | | | | protein family |
| 1506 | HATPase_c | 1/1 | 35-188 | 64.9 | 4.5e−17 | Histidine |
| | | | | | | kinase-, DNA |
| | | | | | | gyrase B-, and |
| | | | | | | HSP90 |
| 1506 | HSP90 | 1/1 | 191-724 | 1284.2 | 0 | Hsp90 protein |
| 1507 | annexin | 1/4 | 35-102 | 88.7 | 1.2e−22 | Annexin |
| 1507 | annexin | 2/4 | 107-174 | 129.7 | 5.3e−35 | Annexin |
| 1507 | annexin | 3/4 | 191-259 | 65.5 | 2.8e−16 | Annexin |
| 1507 | annexin | 4/4 | 267-334 | 108.4 | 1.3e−28 | Annexin |
| 1508 | Ribosomal_L31e | 1/1 | 16-110 | 230.5 | 2.5e−71 | Ribosomal |
| | | | | | | protein L31e |
| 1509 | Glyco_hydro_20 | 1/1 | 200-518 | 724.9 | 3.6e−214 | Glycosyl |
| | | | | | | hydrolase |
| | | | | | | family 20, |
| | | | | | | catalyt |
| 1509 | Glyco_hydro_20b | 1/1 | 68-198 | 245.7 | 6.3e−70 | Glycosyl |
| | | | | | | hydrolase |
| | | | | | | family 20, |
| | | | | | | domain |
| 1510 | proteasome | 1/1 | 50-237 | 183.3 | 2.9e−51 | Proteasome A- |
| | | | | | | type and B-type |
| 1511 | CBS | 1/2 | 112-168 | 30.4 | 1.4e−06 | CBS domain |
| 1511 | CBS | 2/2 | 179-232 | 41.4 | 1.4e−09 | CBS domain |
| 1511 | FMN_dh | 1/1 | 358-387 | 15.5 | 0.00086 | FMN-dependent |
| | | | | | | dehydrogenase |
| 1511 | IMPDH | 1/1 | 20-514 | 683.5 | 1e−201 | IMP |
| | | | | | | dehydrogenase/ |
| | | | | | | GMP reductase |
| 1511 | NPD | 2/2 | 359-397 | 15.5 | 0.0008 | 1/2 52 |
| | | | | | | 91..140 |
| 1512 | Phage_terminase | 1/1 | 44-71 | 3.9 | 0.92 | Phage Terminase |
| 1513 | LRR | 2/2 | 232-256 | 13.7 | 0.05 | Leucine Rich |
| | | | | | | Repeat |
| 1513 | NTF2 | 1/2 | 326-362 | 27.6 | 3.3e−06 | Nuclear |
| | | | | | | transport |
| | | | | | | factor 2 (NTF2) |
| | | | | | | domain |
| 1513 | NTF2 | 2/2 | 397-476 | 36.9 | 9.3e−09 | Nuclear |
| | | | | | | transport |
| | | | | | | factor 2 (NTF2) |
| | | | | | | domain |
| 1513 | TAP_C | 1/1 | 496-559 | 141.0 | 3.9e−39 | TAP C-terminal |
| | | | | | | domain |
| 1514 | CH | 1/1 | 800-905 | 110.2 | 4e−29 | Calponin |
| | | | | | | homology (CH) |
| | | | | | | domain |
| 1515 | ig | 1/1 | 34-115 | 12.6 | 0.079 | Immunoglobulin |
| | | | | | | domain |
| 1516 | synaptobrevin | 1/1 | 2-90 | 118.1 | 1.1e−31 | Synaptobrevin |
| 1517 | zf-MYND | 1/1 | 11-34 | 9.7 | 0.079 | MYND finger |
| 1518 | FAD_binding_6 | 1/1 | 48-155 | 199.9 | 4.1e−56 | Oxidoreductase |
| | | | | | | FAD-binding |
| | | | | | | domain |
| 1518 | NAD_binding_1 | 1/1 | 176-290 | 160.8 | 1.8e−44 | Oxidoreductase |
| | | | | | | NAD-binding |
| | | | | | | domain |
| 1519 | HSP70 | 1/1 | 6-612 | 1407.8 | 0 | Hsp70 protein |
| 1520 | Glyco_hydro_20 | 1/1 | 167-489 | 702.5 | 2e−207 | Glycosyl |
| | | | | | | hydrolase |
| | | | | | | family 20, |
| | | | | | | catalyt |
| 1520 | Glyco_hydro_20b | 1/1 | 35-165 | 215.6 | 7.6e−61 | Glycosyl |
| | | | | | | hydrolase |
| | | | | | | family 20, |
| | | | | | | domain |
| 1521 | rrm | 1/2 | 64-123 | 79.5 | 6e−22 | RNA recognition |
| | | | | | | motif. (a.k.a. |
| | | | | | | RRM, RBD, or |
| 1521 | rrm | 2/2 | 143-207 | 51.8 | 8.7e−14 | RNA recognition |
| | | | | | | motif. (a.k.a. |
| | | | | | | RRM, RBD, or |
| 1522 | ferritin | 1/1 | 14-155 | 194.0 | 2.3e−54 | Ferritin-like |
| | | | | | | domain |
| 1523 | APG9 | 1/1 | 230-608 | 894.2 | 1.7e−268 | Autophagy |
| | | | | | | protein Apg9 |
| 1523 | Diphthamide_syn | 1/1 | 552-577 | 6.9 | 0.32 | Putative |
| | | | | | | diphthamide |
| | | | | | | synthesis |
| | | | | | | protei |
| 1524 | Ribosomal_S15 | 1/1 | 61-151 | 111.8 | 8.6e−31 | Ribosomal |
| | | | | | | protein S15 |
| 1525 | Herpes_HEPA | 1/1 | 10-24 | 3.4 | 0.86 | Herpesvirus DNA |
| | | | | | | helicase/primase |
| | | | | | | complex |
| 1525 | actin | 1/1 | 1-377 | 925.9 | 2.2e−281 | Actin |
| 1526 | aldo_ket_red | 1/1 | 7-296 | 529.8 | 9.4e−156 | Aldo/keto |
| | | | | | | reductase |
| | | | | | | family |
| 1527 | p450 | 1/1 | 48-488 | 355.7 | 1.2e−110 | Cytochrome P450 |
| 1528 | Metallophos | 1/1 | 199-461 | 70.5 | 1.4e−17 | Calcineurin- |
| | | | | | | like |
| | | | | | | phosphoesterase |
| 1529 | WD40 | 1/7 | 5-44 | 34.2 | 6.2e−08 | WD domain, G- |
| | | | | | | beta repeat |
| 1529 | WD40 | 2/7 | 53-91 | 46.5 | 2e−11 | WD domain, G- |
| | | | | | | beta repeat |
| 1529 | WD40 | 3/7 | 95-133 | 50.7 | 1.3e−12 | WD domain, G- |
| | | | | | | beta repeat |
| 1529 | WD40 | 4/7 | 138-178 | 40.3 | 1.2e−09 | WD domain, G- |
| | | | | | | beta repeat |
| 1529 | WD40 | 5/7 | 182-220 | 37.7 | 6.3e−09 | WD domain, G- |
| | | | | | | beta repeat |
| 1529 | WD40 | 6/7 | 224-260 | 22.5 | 0.00013 | WD domain, G- |
| | | | | | | beta repeat |
| 1529 | WD40 | 7/7 | 273-311 | 21.6 | 0.00024 | WD domain, G- |
| | | | | | | beta repeat |
| 1530 | Ribosomal_L7Ae | 1/1 | 20-114 | 114.8 | 7.2e−33 | Ribosomal |
| | | | | | | protein |
| | | | | | | L7Ae/L30e/S12e/ |
| | | | | | | Gadd4 |
| 1531 | cpn60_TCP1 | 1/1 | 32-524 | 583.9 | 9.7e−172 | TCP-1/cpn60 |
| | | | | | | chaperonin |
| | | | | | | family |
| 1532 | CUB | 1/2 | 23-138 | 79.9 | 2.7e−22 | CUB domain |
| 1532 | CUB | 2/2 | 193-302 | 160.5 | 5.2e−46 | CUB domain |
| 1532 | EGF | 1/1 | 161-189 | 17.9 | 0.0023 | EGF-like domain |
| 1532 | sushi | 2/3 | 309-371 | 45.5 | 2.8e−10 | Sushi domain |
| | | | | | | (SCR repeat) |
| 1532 | sushi | 3/3 | 376-447 | 50.7 | 1.2e−11 | Sushi domain |
| | | | | | | (SCR repeat) |
| 1532 | trypsin | 1/1 | 464-697 | 189.7 | 1.7e−59 | Trypsin |
| 1533 | CUB | 1/2 | 18-127 | 71.8 | 6.9e−20 | CUB domain |
| 1533 | CUB | 2/2 | 175-287 | 150.5 | 4.7e−43 | CUB domain |
| 1533 | EGF | 1/1 | 135-171 | 33.5 | 9.3e−08 | EGF-like domain |
| 1533 | sushi | 1/2 | 294-354 | 53.3 | 2.6e−12 | Sushi domain |
| | | | | | | (SCR repeat) |
| 1533 | sushi | 2/2 | 359-421 | 53.5 | 2.2e−12 | Sushi domain |
| | | | | | | (SCR repeat) |
| 1533 | trypsin | 1/1 | 438-675 | 217.2 | 3.1e−68 | Trypsin |
| 1534 | Ndr | 1/1 | 40-322 | 644.1 | 7.5e−190 | Ndr family |
| 1534 | abhydrolase | 1/2 | 95-178 | 11.7 | 0.013 | alpha/beta |
| | | | | | | hydrolase fold |
| 1535 | RrnaAD | 1/1 | 111-147 | 5.2 | 0.78 | Ribosomal RNA |
| | | | | | | adenine |
| | | | | | | dimethylase |
| 1535 | efhand | 1/4 | 12-40 | 39.6 | 4.8e−09 | EF hand |
| 1535 | efhand | 2/4 | 48-76 | 38.4 | 1e−08 | EF hand |
| 1535 | efhand | 3/4 | 85-113 | 42.1 | 9.8e−10 | EF hand |
| 1535 | efhand | 4/4 | 121-149 | 41.2 | 1.7e−09 | EF hand |
| 1536 | calreticulin | 1/1 | 21-332 | 774.0 | 2.2e−238 | Calreticulin |
| | | | | | | family |
| 1537 | S_100 | 1/1 | 5-48 | 88.6 | 1.2e−23 | S-100/ICaBP |
| | | | | | | type calcium |
| | | | | | | binding domain |
| 1537 | efhand | 1/1 | 54-82 | 17.2 | 0.0073 | EF hand |
| 1538 | UPAR_LY6 | 1/1 | 26-96 | 114.1 | 2.7e−30 | u-PAR/Ly-6 |
| | | | | | | domain |
| 1539 | Cys_knot | 1/1 | 22-130 | 187.9 | 1.7e−52 | Cystine-knot |
| | | | | | | domain |
| 1540 | Kunitz_BPTI | 1/3 | 54-104 | 88.8 | 9.4e−28 | Kunitz/Bovine |
| | | | | | | pancreatic |
| | | | | | | trypsin |
| | | | | | | inhibito |
| 1540 | Kunitz_BPTI | 2/3 | 125-175 | 88.2 | 1.5e−27 | Kunitz/Bovine |
| | | | | | | pancreatic |
| | | | | | | trypsin |
| | | | | | | inhibito |
| 1540 | Kunitz_BPTI | 3/3 | 217-267 | 90.8 | 2.1e−28 | Kunitz/Bovine |
| | | | | | | pancreatic |
| | | | | | | trypsin |
| | | | | | | inhibito |
| 1541 | Ribosomal_L13e | 1/1 | 7-185 | 463.8 | 1.4e−135 | Ribosomal |
| | | | | | | protein L13e |
| 1542 | GILT | 1/1 | 62-170 | 88.8 | 1.1e−22 | Gamma |
| | | | | | | interferon |
| | | | | | | inducible |
| | | | | | | lysosomal thiolr |
| 1543 | HSP20 | 1/1 | 60-162 | 164.4 | 2e−45 | Hsp20/alpha |
| | | | | | | crystallin |
| | | | | | | family |
| 1543 | crystallin | 1/1 | 1-59 | 136.2 | 3.7e−38 | Alpha |
| | | | | | | crystallin A |
| | | | | | | chain, N |
| | | | | | | terminal |
| 1544 | CK_II_beta | 1/1 | 5-183 | 508.1 | 6.6e−149 | Casein kinase |
| | | | | | | II regulatory |
| | | | | | | subunit |
| 1545 | fn1 | 1/12 | 81-116 | 64.8 | 1.2e−16 | Fibronectin |
| | | | | | | type I domain |
| 1545 | fn1 | 10/12 | 2204-2243 | 77.5 | 3e−20 | Fibronectin |
| | | | | | | type I domain |
| 1545 | fn1 | 11/12 | 2249-2286 | 72.3 | 9e−19 | Fibronectin |
| | | | | | | type I domain |
| 1545 | fn1 | 12/12 | 2293-2328 | 62.0 | 7.5e−16 | Fibronectin |
| | | | | | | type I domain |
| 1545 | fn1 | 2/12 | 126-164 | 73.9 | 3.1e−19 | Fibronectin |
| | | | | | | type I domain |
| 1545 | fn1 | 3/12 | 170-208 | 67.9 | 1.5e−17 | Fibronectin |
| | | | | | | type I domain |
| 1545 | fn1 | 4/12 | 215-254 | 80.4 | 4.5e−21 | Fibronectin |
| | | | | | | type I domain |
| 1545 | fn1 | 5/12 | 260-299 | 76.9 | 4.4e−20 | Fibronectin |
| | | | | | | type I domain |
| 1545 | fn1 | 6/12 | 337-371 | 51.4 | 7.8e−13 | Fibronectin |
| | | | | | | type I domain |
| 1545 | fn1 | 7/12 | 499-537 | 77.0 | 4.2e−20 | Fibronectin |
| | | | | | | type I domain |
| 1545 | fn1 | 8/12 | 547-584 | 63.8 | 2.3e−16 | Fibronectin |
| | | | | | | type I domain |
| 1545 | fn1 | 9/12 | 590-628 | 71.3 | 1.7e−18 | Fibronectin |
| | | | | | | type I domain |
| 1545 | fn2 | 1/2 | 389-430 | 94.2 | 7.6e−35 | Fibronectin |
| | | | | | | type II domain |
| 1545 | fn2 | 2/2 | 449-490 | 100.1 | 4.1e−37 | Fibronectin |
| | | | | | | type II domain |
| 1545 | fn3 | 1/16 | 637-720 | 53.0 | 7.5e−14 | Fibronectin |
| | | | | | | type III domain |
| 1545 | fn3 | 10/16 | 1477-1559 | 101.6 | 7e−28 | Fibronectin |
| | | | | | | type III domain |
| 1545 | fn3 | 11/16 | 1571-1650 | 81.6 | 4.1e−22 | Fibronectin |
| | | | | | | type III domain |
| 1545 | fn3 | 12/16 | 1661-1740 | 62.3 | 1.5e−16 | Fibronectin |
| | | | | | | type III domain |
| 1545 | fn3 | 13/16 | 1751-1830 | 79.0 | 2.3e−21 | Fibronectin |
| | | | | | | type III domain |
| 1545 | fn3 | 14/16 | 1843-1921 | 83.0 | 1.7e−22 | Fibronectin |
| | | | | | | type III domain |
| 1545 | fn3 | 15/16 | 1932-2011 | 92.8 | 2.4e−25 | Fibronectin |
| | | | | | | type III domain |
| 1545 | fn3 | 16/16 | 2102-2179 | 31.5 | 1.2e−07 | Fibronectin |
| | | | | | | type III domain |
| 1545 | fn3 | 2/16 | 749-829 | 59.7 | 8.8e−16 | Fibronectin |
| | | | | | | type III domain |
| 1545 | fn3 | 3/16 | 840-918 | 96.0 | 2.8e−26 | Fibronectin |
| | | | | | | type III domain |
| 1545 | fn3 | 4/16 | 936-1015 | 84.6 | 5.8e−23 | Fibronectin |
| | | | | | | type III domain |
| 1545 | fn3 | 5/16 | 1026-1104 | 80.6 | 8.3e−22 | Fibronectin |
| | | | | | | type III domain |
| 1545 | fn3 | 6/16 | 1125-1191 | 32.6 | 5.9e−08 | Fibronectin |
| | | | | | | type III domain |
| 1545 | fn3 | 7/16 | 1203-1285 | 74.5 | 4.7e−20 | Fibronectin |
| | | | | | | type III domain |
| 1545 | fn3 | 8/16 | 1296-1376 | 82.9 | 1.8e−22 | Fibronectin |
| | | | | | | type III domain |
| 1545 | fn3 | 9/16 | 1387-1466 | 86.1 | 2.2e−23 | Fibronectin |
| | | | | | | type III domain |
| 1546 | p450 | 1/1 | 52-511 | 482.4 | 2.7e−150 | Cytochrome P450 |
| 1548 | GTP_EFTU | 1/1 | 5-239 | 376.4 | 2.9e−109 | Elongation |
| | | | | | | factor Tu GTP |
| | | | | | | binding domain |
| 1548 | GTP_EFTU_D2 | 1/1 | 251-327 | 100.6 | 8e−27 | Elongation |
| | | | | | | factor Tu |
| | | | | | | domain 2 |
| 1548 | GTP_EFTU_D3 | 1/1 | 333-442 | 213.3 | 3.5e−60 | Elongation |
| | | | | | | factor Tu C- |
| | | | | | | terminal domain |
| 1549 | fibrinogen_C | 1/1 | 175-415 | 532.0 | 4.2e−156 | Fibrinogen beta |
| | | | | | | and gamma |
| | | | | | | chains, C-t |
| 1550 | PX | 1/1 | 65-178 | 106.7 | 4.6e−28 | PX domain |
| 1551 | lectin_c | 1/1 | 114-222 | 48.8 | 2.9e−11 | Lectin C-type |
| | | | | | | domain |
| 1552 | lipocalin | 1/1 | 32-178 | 129.6 | 1.8e−36 | Lipocalin/ |
| | | | | | | cytosolic |
| | | | | | | fatty-acid |
| | | | | | | binding pr |
| 1553 | enolase | 1/1 | 142-432 | 717.1 | 8.3e−212 | Enolase, C- |
| | | | | | | terminal TIM |
| | | | | | | barrel domain |
| 1553 | enolase_N | 1/1 | 2-134 | 280.0 | 3.1e−80 | Enolase, N- |
| | | | | | | terminal domain |
| 1554 | p450 | 1/1 | 44-498 | 543.8 | 1.6e−169 | Cytochrome P450 |
| 1555 | Ribosomal_S3Ae | 1/1 | 12-222 | 518.5 | 5e−152 | Ribosomal S3Ae |
| | | | | | | family |
| 1556 | ldl_recept_a | 1/3 | 34-61 | 24.8 | 5.7e−06 | Low-density |
| | | | | | | lipoprotein |
| | | | | | | receptor domain |
| 1556 | ldl_recept_a | 3/3 | 105-141 | 45.1 | 3.8e−12 | Low-density |
| | | | | | | lipoprotein |
| | | | | | | receptor domain |
| 1557 | Glyco_hydro_31 | 1/1 | 194-918 | 1402.0 | 0 | Glycosyl |
| | | | | | | hydrolases |
| | | | | | | family 31 |
| 1557 | trefoil | 1/1 | 81-130 | 53.4 | 2.2e−13 | Trefoil (P- |
| | | | | | | type) domain |
| 1558 | globin | 1/1 | 3-147 | 218.1 | 1.3e−61 | Globin |
| 1559 | ANATO | 1/1 | 96-102 | 5.5 | 0.18 | Anaphylotoxin- |
| | | | | | | like domain |
| 1559 | fn3 | 3/4 | 429-516 | 13.0 | 0.027 | Fibronectin |
| | | | | | | type III domain |
| 1559 | fn3 | 4/4 | 527-609 | 33.7 | 2.8e−08 | Fibronectin |
| | | | | | | type III domain |
| 1560 | IGFBP | 1/1 | 40-99 | 97.4 | 2e−26 | Insulin-like |
| | | | | | | growth factor |
| | | | | | | binding pr |
| 1560 | thyroglobulin_1 | 1/1 | 213-285 | 119.6 | 5.9e−32 | Thyroglobulin |
| | | | | | | type-1 repeat |
| 1561 | IGFBP | 1/1 | 30-89 | 86.8 | 2.1e−23 | Insulin-like |
| | | | | | | growth factor |
| | | | | | | binding pr |
| 1561 | thyroglobulin_1 | 1/1 | 176-251 | 144.3 | 2.1e−39 | Thyroglobulin |
| | | | | | | type-1 repeat |
| 1562 | histone | 1/1 | 17-135 | 100.3 | 3.6e−27 | Core histone |
| | | | | | | H2A/H2B/H3/H4 |
| 1563 | MHC_I | 1/1 | 22-200 | 473.5 | 1.7e−138 | Class I |
| | | | | | | Histocompatibility |
| | | | | | | antigen, |
| | | | | | | domains |
| 1563 | ig | 1/1 | 217-282 | 36.6 | 1.7e−08 | Immunoglobulin |
| | | | | | | domain |
| 1564 | AT_hook | 1/1 | 244-256 | 12.0 | 0.12 | AT hook motif |
| 1564 | Ets | 1/1 | 272-357 | 127.6 | 8.8e−35 | Ets-domain |
| 1564 | SAM_PNT | 1/1 | 48-132 | 123.5 | 4.9e−35 | Sterile alpha |
| | | | | | | motif |
| | | | | | | (SAM)/Pointed |
| | | | | | | domain |
| 1565 | MHC_I | 1/1 | 25-203 | 481.8 | 5.6e−141 | Class I |
| | | | | | | Histocompatibility |
| | | | | | | antigen, |
| | | | | | | domains |
| 1565 | ig | 1/1 | 220-285 | 33.3 | 1.4e−07 | Immunoglobulin |
| | | | | | | domain |
| 1566 | ubiquitin | 1/2 | 3-78 | 28.4 | 2e−06 | Ubiquitin |
| | | | | | | family |
| 1566 | ubiquitin | 2/2 | 82-155 | 84.4 | 3.8e−22 | Ubiquitin |
| | | | | | | family |
| 1567 | C2 | 1/2 | 310-396 | 92.2 | 3.8e−25 | C2 domain |
| 1567 | C2 | 2/2 | 461-539 | 30.4 | 2.6e−07 | C2 domain |
| 1568 | EMP24_GP25L | 1/1 | 8-211 | 142.6 | 7.2e−39 | emp24/gp25L/p24 |
| | | | | | | family |
| 1569 | DSPc | 1/1 | 173-311 | 261.6 | 1.1e−74 | Dual |
| | | | | | | specificity |
| | | | | | | phosphatase, |
| | | | | | | catalytic |
| 1569 | Rhodanese | 1/1 | 9-131 | 92.1 | 6e−26 | Rhodanese-like |
| | | | | | | domain |
| 1569 | Y_phosphatase | 1/1 | 237-290 | 19.8 | 0.00013 | Protein- |
| | | | | | | tyrosine |
| | | | | | | phosphatase |
| 1570 | Ribosomal_S17e | 1/1 | 1-122 | 305.6 | 2.5e−95 | Ribosomal S17 |
| 1571 | proteasome | 1/1 | 29-215 | 145.9 | 1.4e−40 | Proteasome A- |
| | | | | | | type and B-type |
| 1572 | TCTP | 1/1 | 1-135 | 287.4 | 4.2e−87 | Translationally |
| | | | | | | controlled |
| | | | | | | tumor prot |
| 1573 | HATPase_c | 1/1 | 1-96 | 27.5 | 1.8e−06 | Histidine |
| | | | | | | kinase-, DNA |
| | | | | | | gyrase B-, and H |
| 1573 | HSP90 | 1/1 | 99-635 | 1270.1 | 0 | Hsp90 protein |
| 1574 | Alpha_adaptin_C | 1/1 | 226-237 | 6.3 | 0.25 | Alpha adaptin |
| | | | | | | AP2, C-terminal |
| | | | | | | domain |
| 1574 | Amino_oxidase | 1/1 | 23-460 | 326.9 | 2.3e−94 | Flavin |
| | | | | | | containing |
| | | | | | | amine |
| | | | | | | oxidoreductas |
| 1574 | DAO | 1/1 | 14-46 | 21.0 | 9.2e−05 | FAD dependent |
| | | | | | | oxidoreductase |
| 1574 | FAD_binding_3 | 1/1 | 16-48 | 31.1 | 7.2e−09 | FAD binding |
| | | | | | | domain |
| 1574 | GMC_oxred | 1/1 | 14-44 | 8.1 | 0.11 | GMC |
| | | | | | | oxidoreductase |
| 1574 | Thi4 | 1/1 | 15-45 | 14.8 | 0.00093 | Thi4 family |
| 1574 | TrkA-N | 1/1 | 15-43 | 13.2 | 0.037 | TrkA-N domain |
| 1574 | pyr_redox | 1/1 | 15-50 | 21.8 | 1.6e−05 | Pyridine |
| | | | | | | nucleotide- |
| | | | | | | disulphide |
| | | | | | | oxidor |
| 1575 | MHC_I | 1/1 | 25-203 | 482.4 | 3.5e−141 | Class I |
| | | | | | | Histocompatibility |
| | | | | | | antigen, |
| | | | | | | domains |
| 1575 | ig | 1/1 | 220-285 | 36.3 | 2.2e−08 | Immunoglobulin |
| | | | | | | domain |
| 1576 | MHC_II_alpha | 1/1 | 29-109 | 158.0 | 1.4e−46 | Class II |
| | | | | | | histocompatibility |
| | | | | | | antigen, |
| | | | | | | alp |
| 1576 | ig | 1/1 | 125-190 | 32.0 | 3.4e−07 | Immunoglobulin |
| | | | | | | domain |
| 1577 | NDK | 1/1 | 5-152 | 396.9 | 2e−115 | Nucleoside |
| | | | | | | diphosphate |
| | | | | | | kinase |
| 1578 | AAA | 1/1 | 140-157 | 12.4 | 0.01 | ATPase family |
| | | | | | | associated with |
| | | | | | | various ce |
| 1578 | ras | 1/2 | 140-154 | 14.4 | 0.0034 | Ras family |
| 1578 | ras | 2/2 | 213-321 | 96.7 | 1.2e−27 | Ras family |
| 1579 | Ribosomal_L10e | 1/1 | 1-176 | 465.4 | 3.7e−138 | Ribosomal L10 |
| 1580 | RHD | 1/1 | 18-186 | 416.3 | 6.5e−125 | Rel homology |
| | | | | | | domain (RHD) |
| 1580 | TIG | 1/1 | 194-289 | 80.1 | 4.5e−20 | IPT/TIG domain |
| 1581 | serpin | 1/1 | 28-402 | 583.7 | 5.8e−172 | Serpin (serine |
| | | | | | | protease |
| | | | | | | inhibitor) |
| 1582 | PCNA | 1/1 | 1-125 | 319.7 | 3.3e−92 | Proliferating |
| | | | | | | cell nuclear |
| | | | | | | antigen, N- |
| | | | | | | termin |
| 1582 | PCNA_C | 1/1 | 127-254 | 317.2 | 1.9e−91 | Proliferating |
| | | | | | | cell nuclear |
| | | | | | | antigen, C- |
| | | | | | | termin |
| 1583 | Herpes_glycop_D | 1/1 | 352-386 | 7.7 | 0.43 | Herpesvirus |
| | | | | | | glycoprotein D |
| 1583 | ig | 1/3 | 43-110 | 19.4 | 0.001 | Immunoglobulin |
| | | | | | | domain |
| 1583 | ig | 2/3 | 165-209 | 18.5 | 0.0018 | Immunoglobulin |
| | | | | | | domain |
| 1583 | ig | 3/3 | 251-328 | 26.9 | 8.5e−06 | Immunoglobulin |
| | | | | | | domain |
| 1584 | WD40 | 2/6 | 84-123 | 9.0 | 0.94 | WD domain, G- |
| | | | | | | beta repeat |
| 1585 | ig | 1/4 | 85-121 | 10.1 | 0.39 | Immunoglobulin |
| | | | | | | domain |
| 1585 | ig | 3/4 | 255-312 | 16.3 | 0.0076 | Immunoglobulin |
| | | | | | | domain |
| 1585 | ig | 4/4 | 347-396 | 32.3 | 2.7e−07 | Immunoglobulin |
| | | | | | | domain |
| 1586 | lipocalin | 1/1 | 4-134 | 110.6 | 6e−31 | Lipocalin/ |
| | | | | | | cytosolic |
| | | | | | | fatty-acid |
| | | | | | | binding pr |
| 1587 | Grp1_Fun34_YaaH | 1/1 | 557-575 | 6.3 | 0.3 | GPR1/FUN34/yaaH |
| | | | | | | family |
| 1587 | IGA1 | 1/1 | 63-81 | 5.6 | 0.59 | Immunoglobulin |
| | | | | | | A1 protease |
| 1587 | ig | 1/4 | 35-85 | 13.5 | 0.043 | Immunoglobulin |
| | | | | | | domain |
| 1587 | ig | 2/4 | 217-280 | 31.4 | 4.9e−07 | Immunoglobulin |
| | | | | | | domain |
| 1587 | ig | 4/4 | 412-487 | 22.3 | 0.00016 | Immunoglobulin |
| | | | | | | domain |
| 1587 | pkinase | 1/1 | 582-910 | 253.7 | 2.5e−72 | Protein kinase |
| | | | | | | domain |
| 1588 | RNA_pol_Rpb7_N | 1/1 | 1-77 | 135.3 | 2.5e−39 | RNA polymerase |
| | | | | | | Rpb7, N- |
| | | | | | | terminal domain |
| 1588 | S1 | 1/1 | 78-161 | 69.4 | 1.4e−17 | S1 RNA binding |
| | | | | | | domain |
| 1590 | GATase | 1/1 | 204-249 | 8.3 | 0.33 | Glutamine |
| | | | | | | amidotransferase |
| | | | | | | class-I |
| 1591 | Ribosomal_S2 | 1/1 | 18-186 | 340.5 | 1.8e−98 | Ribosomal |
| | | | | | | protein S2 |
| 1592 | Gelsolin | 1/6 | 76-126 | 64.7 | 6.4e−17 | Gelsolin repeat |
| 1592 | Gelsolin | 2/6 | 198-238 | 70.6 | 1.3e−18 | Gelsolin repeat |
| 1592 | Gelsolin | 3/6 | 314-356 | 62.2 | 3.2e−16 | Gelsolin repeat |
| 1592 | Gelsolin | 4/6 | 453-504 | 73.6 | 1.7e−19 | Gelsolin repeat |
| 1592 | Gelsolin | 5/6 | 576-616 | 42.9 | 1.1e−10 | Gelsolin repeat |
| 1592 | Gelsolin | 6/6 | 679-721 | 63.4 | 1.5e−16 | Gelsolin repeat |
| 1593 | ion_trans | 1/1 | 239-410 | 59.6 | 5.4e−15 | Ion transport |
| | | | | | | protein |
| 1594 | ig | 1/2 | 148-206 | 24.5 | 4.1e−05 | Immunoglobulin |
| | | | | | | domain |
| 1594 | ig | 2/2 | 256-310 | 25.3 | 2.4e−05 | Immunoglobulin |
| | | | | | | domain |
| 1595 | DUF258 | 1/1 | 15-30 | 6.4 | 0.79 | Protein of |
| | | | | | | unknown |
| | | | | | | function, |
| | | | | | | DUF258 |
| 1595 | ras | 1/1 | 13-218 | 366.4 | 9.7e−108 | Ras family |
| 1596 | Exonuc_V_gamma | 1/1 | 187-229 | 5.0 | 0.51 | Exodeoxyribonuclease |
| | | | | | | V, gamma |
| | | | | | | subunit |
| 1596 | Ribosomal_S6e | 1/1 | 1-127 | 312.1 | 6.7e−90 | Ribosomal |
| | | | | | | protein S6e |
| 1597 | IL8 | 1/1 | 23-90 | 47.3 | 6.7e−12 | Small cytokines |
| | | | | | | (intecrine/chemokine), |
| | | | | | | inter |
| 1598 | Exo_endo_phos | 1/1 | 8-237 | 141.2 | 1.9e−38 | Endonuclease/Exonuclease/ |
| | | | | | | phosphatase |
| | | | | | | fa |
| 1598 | rvt | 1/1 | 508-773 | 263.0 | 3.9e−75 | Reverse |
| | | | | | | transcriptase |
| | | | | | | (RNA-dependent |
| | | | | | | DN |
| 1599 | Mov34 | 1/1 | 26-139 | 173.8 | 2.9e−48 | Mov34/MPN/PAD-1 |
| | | | | | | family |
| 1600 | histone | 1/1 | 19-137 | 100.3 | 3.6e−27 | Core histone |
| | | | | | | H2A/H2B/H3/H4 |
| 1601 | 2-Hacid_DH | 1/1 | 44-137 | 133.7 | 2.6e−36 | D-isomer |
| | | | | | | specific 2- |
| | | | | | | hydroxyacid |
| | | | | | | dehydrog |
| 1601 | 2-Hacid_DH_C | 1/1 | 139-320 | 199.8 | 4.1e−81 | D-isomer |
| | | | | | | specific 2- |
| | | | | | | hydroxyacid |
| | | | | | | dehydrog |
| 1601 | PDH | 1/1 | 187-213 | 8.1 | 0.35 | Prephenate |
| | | | | | | dehydrogenase |
| 1602 | GST_C | 1/1 | 127-217 | 11.9 | 0.048 | Glutathione S- |
| | | | | | | transferase, C- |
| | | | | | | terminal |
| 1603 | proteasome | 1/1 | 31-217 | 238.3 | 1.1e−67 | Proteasome A- |
| | | | | | | type and B-type |
| 1604 | Ribosomal_S8 | 1/1 | 5-130 | 192.1 | 6.6e−58 | Ribosomal |
| | | | | | | protein S8 |
| 1605 | PAE | 1/1 | 17-102 | 7.4 | 0.2 | Pectinacetylest |
| | | | | | | erase |
| 1606 | PCI | 1/1 | 328-408 | 80.5 | 7.2e−22 | PCI domain |
| 1607 | Ribosomal_L6e | 1/1 | 181-288 | 251.0 | 8e−73 | Ribosomal |
| | | | | | | protein L6e |
| 1607 | Ribosomal_L6e_N | 1/1 | 34-96 | 128.0 | 1.2e−36 | Ribosomal |
| | | | | | | protein L6, N- |
| | | | | | | terminal doma |
| 1608 | EF1G_domain | 1/1 | 275-381 | 303.5 | 2.3e−87 | Elongation |
| | | | | | | factor 1 gamma, |
| | | | | | | conserved dom |
| 1608 | GST_C | 1/1 | 86-198 | 113.3 | 8.8e−31 | Glutathione S- |
| | | | | | | transferase, C- |
| | | | | | | terminal do |
| 1608 | GST_N | 1/1 | 1-81 | 89.1 | 1.9e−24 | Glutathione S- |
| | | | | | | transferase, N- |
| | | | | | | terminal do |
| 1609 | CH | 1/1 | 25-138 | 75.3 | 2e−19 | Calponin |
| | | | | | | homology (CH) |
| | | | | | | domain |
| 1609 | calponin | 1/1 | 175-200 | 51.8 | 1.7e−14 | Calponin family |
| | | | | | | repeat |
| 1610 | E7 | 1/1 | 1561-1570 | 6.3 | 0.41 | E7 protein, |
| | | | | | | Early protein |
| 1610 | Glyco_hydro_16 | 1/1 | 1773-1802 | 9.3 | 0.15 | Glycosyl |
| | | | | | | hydrolases |
| | | | | | | family 16 |
| 1610 | Mov34 | 1/1 | 2099-2205 | 99.9 | 5e−26 | Mov34/MPN/PAD-1 |
| | | | | | | family |
| 1610 | toxin_3 | 1/2 | 955-973 | 8.9 | 0.15 | |
| 1611 | ATP-bind | 1/1 | 488-526 | 6.9 | 0.49 | Conserved |
| | | | | | | hypothetical |
| | | | | | | ATP binding |
| | | | | | | protei |
| 1611 | Ku_C | 1/1 | 471-559 | 143.1 | 4.9e−39 | Ku70/Ku80 C- |
| | | | | | | terminal arm |
| 1611 | Ku_N | 1/1 | 37-256 | 337.7 | 1.3e−97 | Ku70/Ku80 N- |
| | | | | | | terminal |
| | | | | | | alpha/beta |
| | | | | | | domain |
| 1611 | SAP | 1/1 | 573-607 | 39.5 | 1.5e−09 | SAP domain |
| 1611 | ku | 1/1 | 261-468 | 296.2 | 2.7e−92 | Ku70/Ku80 beta- |
| | | | | | | barrel domain |
| 1612 | Nop | 1/1 | 197-345 | 201.6 | 1.2e−56 | Putative snoRNA |
| | | | | | | binding domain |
| 1614 | 3Beta_HSD | 1/2 | 5-26 | 8.8 | 0.15 | 3-beta |
| | | | | | | hydroxysteroid |
| | | | | | | dehydrogenase/i |
| 1614 | 3Beta_HSD | 2/2 | 84-135 | 8.4 | 0.18 | 3-beta |
| | | | | | | hydroxysteroid |
| | | | | | | dehydrogenase/i |
| 1614 | Epimerase | 1/1 | 5-341 | 499.3 | 3e−146 | NAD dependent |
| | | | | | | epimerase/dehydratase f |
| 1614 | adh_short | 1/1 | 1-33 | 11.7 | 0.029 | short chain |
| | | | | | | dehydrogenase |
| 1616 | ATP-synt_C | 1/1 | 67-135 | 107.9 | 1.9e−28 | ATP synthase |
| | | | | | | subunit C |
| 1618 | Flavi_NS2A | 1/1 | 332-354 | 8.3 | 0.74 | Flavivirus non- |
| | | | | | | structural |
| | | | | | | protein NS2A |
| 1618 | Lamp | 1/1 | 1-354 | 612.3 | 2.8e−180 | Lysosome- |
| | | | | | | associated |
| | | | | | | membrane |
| | | | | | | glycoprote |
| 1618 | Peptidase_M50 | 1/1 | 3-341 | 6.7 | 0.82 | Peptidase |
| | | | | | | family M50 |
| 1619 | A4_EXTRA | 1/1 | 42-204 | 413.4 | 2e−209 | Amyloid A4 |
| | | | | | | extracellular |
| | | | | | | domain |
| 1619 | Coprogen_oxidas | 1/1 | 164-182 | 5.4 | 0.13 | Coproporphyrinogen |
| | | | | | | III oxidase |
| 1619 | Kunitz_BPTI | 1/1 | 310-360 | 95.4 | 6.6e−30 | Kunitz/Bovine |
| | | | | | | pancreatic |
| | | | | | | trypsin inhi |
| 1620 | Ribosomal_L30 | 1/1 | 88-140 | 98.3 | 2.3e−28 | Ribosomal |
| | | | | | | protein |
| | | | | | | L30p/L7e |
| 1621 | PX | 1/1 | 26-148 | 128.2 | 1.5e−34 | PX domain |
| 1622 | ig | 1/7 | 90-109 | 9.4 | 0.59 | Immunoglobulin |
| | | | | | | domain |
| 1622 | ig | 3/7 | 245-313 | 31.5 | 4.5e−07 | Immunoglobulin |
| | | | | | | domain |
| 1622 | ig | 4/7 | 359-404 | 16.6 | 0.006 | Immunoglobulin |
| | | | | | | domain |
| 1622 | ig | 6/7 | 570-638 | 31.6 | 4.4e−07 | Immunoglobulin |
| | | | | | | domain |
| 1622 | ig | 7/7 | 675-733 | 45.6 | 5.8e−11 | Immunoglobulin |
| | | | | | | domain |
| 1622 | pkinase | 1/1 | 827-1152 | 259.7 | 3.8e−74 | Protein kinase |
| | | | | | | domain |
| 1624 | zf-C3HC4 | 1/1 | 521-560 | 8.8 | 0.21 | Zinc finger, |
| | | | | | | C3HC4 type |
| | | | | | | (RING finger) |
| 1625 | hormone6 | 1/1 | 21-116 | 202.4 | 7e−57 | Glycoprotein |
| | | | | | | hormone |
| 1626 | G-alpha | 1/1 | 13-393 | 687.4 | 7.2e−203 | G-protein alpha |
| | | | | | | subunit |
| 1626 | arf | 1/1 | 213-296 | 26.1 | 2.9e−06 | ADP- |
| | | | | | | ribosylation |
| | | | | | | factor family |
| 1627 | rrm | 1/2 | 16-86 | 84.2 | 2.5e−23 | RNA recognition |
| | | | | | | motif. (a.k.a. |
| | | | | | | RRM, RBD, or |
| 1627 | rrm | 2/2 | 107-177 | 91.0 | 2.4e−25 | RNA recognition |
| | | | | | | motif. (a.k.a. |
| | | | | | | RRM, RBD, or |
| 1628 | HMG_box | 1/1 | 1-36 | 34.8 | 3.8e−08 | HMG (high |
| | | | | | | mobility group) |
| | | | | | | box |
| 1629 | OEP | 1/1 | 92-130 | 7.6 | 0.42 | Outer membrane |
| | | | | | | efflux protein |
| 1629 | Ribosomal_L7Ae | 1/1 | 122-216 | 102.0 | 4.7e−29 | Ribosomal |
| | | | | | | protein |
| | | | | | | L7Ae/L30e/S12e/ |
| | | | | | | Gadd4 |
| 1630 | Ribosomal_S24e | 1/1 | 23-105 | 192.0 | 8.1e−55 | Ribosomal |
| | | | | | | protein S24e |
| 1631 | SAPA | 1/2 | 21-54 | 85.7 | 5.9e−33 | Saposin A-type |
| | | | | | | domain |
| 1631 | SAPA | 2/2 | 491-524 | 78.1 | 6.4e−30 | Saposin A-type |
| | | | | | | domain |
| 1631 | Surfactant_B | 2/3 | 357-389 | 9.8 | 0.1 | 1/3 240 |
| | | | | | | 272 . . . 4578 |
| 1632 | Tropomyosin | 2/2 | 48-284 | 501.7 | 5.5e−147 | Tropomyosin |
| 1633 | hormone | 1/1 | 12-227 | 352.4 | 2.1e−130 | Somatotropin |
| | | | | | | hormone family |
| 1635 | DSPc | 2/2 | 1806-1840 | 8.7 | 0.36 | Dual |
| | | | | | | specificity |
| | | | | | | phosphatase, |
| | | | | | | catalytic |
| 1635 | Y_phosphatase | 1/2 | 1365-1596 | 454.3 | 1.1e−132 | Protein- |
| | | | | | | tyrosine |
| | | | | | | phosphatase |
| 1635 | Y_phosphatase | 2/2 | 1654-1887 | 413.6 | 1.8e−120 | Protein- |
| | | | | | | tyrosine |
| | | | | | | phosphatase |
| 1635 | fn3 | 1/8 | 309-391 | 73.7 | 8e−20 | Fibronectin |
| | | | | | | type III domain |
| 1635 | fn3 | 2/8 | 403-490 | 66.5 | 9.6e−18 | Fibronectin |
| | | | | | | type III domain |
| 1635 | fn3 | 3/8 | 502-584 | 70.5 | 6.7e−19 | Fibronectin |
| | | | | | | type III domain |
| 1635 | fn3 | 4/8 | 596-686 | 69.3 | 1.5e−18 | Fibronectin |
| | | | | | | type III domain |
| 1635 | fn3 | 5/8 | 698-799 | 58.7 | 1.7e−15 | Fibronectin |
| | | | | | | type III domain |
| 1635 | fn3 | 6/8 | 811-894 | 50.4 | 4.3e−13 | Fibronectin |
| | | | | | | type III domain |
| 1635 | fn3 | 7/8 | 905-990 | 67.0 | 6.8e−18 | Fibronectin |
| | | | | | | type III domain |
| 1635 | ig | 1/3 | 37-99 | 29.7 | 1.4e−06 | Immunoglobulin |
| | | | | | | domain |
| 1635 | ig | 2/3 | 139-199 | 23.2 | 9.1e−05 | Immunoglobulin |
| | | | | | | domain |
| 1635 | ig | 3/3 | 236-290 | 26.7 | 1e−05 | Immunoglobulin |
| | | | | | | domain |
| 1636 | ATP-synt_ab | 1/1 | 138-421 | 550.0 | 1.5e−161 | ATP synthase |
| | | | | | | alpha/beta |
| | | | | | | family, nucleot |
| 1636 | ATP-synt_ab_C | 1/1 | 423-531 | 138.7 | 9.9e−39 | ATP synthase |
| | | | | | | alpha/beta |
| | | | | | | chain, C termin |
| 1636 | ATP-synt_ab_N | 1/1 | 67-135 | 74.5 | 1e−23 | ATP synthase |
| | | | | | | alpha/beta |
| | | | | | | family, beta-ba |
| 1637 | S_100 | 1/1 | 8-51 | 87.9 | 1.9e−23 | S-100/ICaBP |
| | | | | | | type calcium |
| | | | | | | binding domain |
| 1637 | efhand | 1/1 | 58-86 | 23.8 | 0.00011 | EF hand |
| 1638 | GLTT | 1/1 | 40-68 | 7.6 | 0.44 | GLTT repeat (6 |
| | | | | | | copies) |
| 1639 | PGK | 1/1 | 2-418 | 1017.1 | 3.9e−302 | Phosphoglycerate |
| | | | | | | kinase |
| 1640 | C2 | 1/8 | 2-85 | 87.2 | 1.1e−23 | C2 domain |
| 1640 | C2 | 2/8 | 222-302 | 52.5 | 1.1e−13 | C2 domain |
| 1640 | C2 | 3/8 | 381-479 | 66.0 | 1.4e−17 | C2 domain |
| 1640 | C2 | 4/8 | 1154-1244 | 90.3 | 1.3e−24 | C2 domain |
| 1640 | C2 | 5/8 | 1338-1421 | 17.4 | 0.0014 | C2 domain |
| 1640 | C2 | 6/8 | 1580-1663 | 79.2 | 2.2e−21 | C2 domain |
| 1640 | C2 | 8/8 | 1904-1926 | 15.3 | 0.0059 | C2 domain |
| 1641 | RNA_pol_A | 1/2 | 246-895 | 1289.3 | 0 | RNA polymerase |
| | | | | | | alpha subunit |
| 1641 | RNA_pol_A2 | 1/1 | 1060-1477 | 762.0 | 2.4e−225 | RNA polymerase |
| | | | | | | A/beta′/A″ |
| | | | | | | subunit |
| 1643 | HATPase_c | 1/1 | 96-254 | 54.7 | 3.4e−14 | Histidine |
| | | | | | | kinase-, DNA |
| | | | | | | gyrase B-, and |
| | | | | | | HSP90 |
| 1643 | HSP90 | 1/1 | 257-783 | 1105.0 | 0 | Hsp90 protein |
| 1644 | Ribosomal_L40e | 1/1 | 77-128 | 111.5 | 1.6e−29 | Ribosomal L40e |
| | | | | | | family |
| 1644 | ubiquitin | 1/1 | 1-74 | 153.1 | 4.8e−42 | Ubiquitin |
| | | | | | | family |
| 1646 | Ribosomal_S13 | 1/1 | 14-142 | 274.7 | 1.2e−78 | Ribosomal |
| | | | | | | protein S13/S18 |
| 1647 | Ribosomal_L5 | 1/1 | 10-63 | 87.8 | 2.2e−22 | Ribosomal |
| | | | | | | protein L5 |
| 1647 | Ribosomal_L5_C | 1/1 | 67-166 | 210.1 | 1.7e−62 | ribosomal L5P |
| | | | | | | family C- |
| | | | | | | terminus |
| 1648 | Armadillo_seg | 1/2 | 1017-1044 | 10.7 | 0.3 | Armadillo/beta- |
| | | | | | | catenin-like |
| | | | | | | repeat |
| 1649 | ig | 1/1 | 38-102 | 33.8 | 1e−07 | Immunoglobulin |
| | | | | | | domain |
| 1650 | ICE_p10 | 1/1 | 244-329 | 180.7 | 2.3e−50 | ICE-like |
| | | | | | | protease |
| | | | | | | (caspase) p10 |
| | | | | | | domain |
| 1650 | ICE_p20 | 1/1 | 88-219 | 293.9 | 5.7e−88 | ICE-like |
| | | | | | | protease |
| | | | | | | (caspase) p20 |
| | | | | | | domain |
| 1650 | TAF | 1/1 | 133-143 | 4.8 | 1 | TATA box |
| | | | | | | binding protein |
| | | | | | | associated |
| | | | | | | factor ( |
| 1651 | Ribosomal_L6 | 1/2 | 12-87 | 84.5 | 1.9e−22 | Ribosomal |
| | | | | | | protein L6 |
| 1651 | Ribosomal_L6 | 2/2 | 99-178 | 66.8 | 2e−17 | Ribosomal |
| | | | | | | protein L6 |
| 1652 | Ribosomal_S12 | 1/1 | 7-142 | 279.8 | 3.4e−80 | Ribosomal |
| | | | | | | protein S12 |
| 1653 | ubiquitin | 1/1 | 1-74 | 116.3 | 4.1e−31 | Ubiquitin |
| | | | | | | family |
| 1654 | Fragilysin | 1/1 | 212-238 | 11.4 | 0.019 | Fragilysin |
| | | | | | | metallopeptidase |
| | | | | | | (M10C) en |
| 1654 | Peptidase_M10 | 1/1 | 98-204 | 163.6 | 3.3e−45 | Matrixin |
| 1654 | Peptidase_M10_N | 1/1 | 75-84 | 10.2 | 0.014 | Matrix |
| | | | | | | metalloprotease, |
| | | | | | | N-terminal do |
| 1654 | hemopexin | 1/4 | 298-341 | 58.5 | 3.3e−16 | Hemopexin |
| 1654 | hemopexin | 2/4 | 343-384 | 48.6 | 3.6e−13 | Hemopexin |
| 1654 | hemopexin | 3/4 | 387-434 | 54.2 | 7.1e−15 | Hemopexin |
| 1654 | hemopexin | 4/4 | 436-480 | 53.4 | 1.3e−14 | Hemopexin |
| 1655 | Sec61_beta | 1/1 | 49-94 | 98.9 | 2.4e−26 | Sec61beta |
| | | | | | | family |
| 1656 | ras | 1/1 | 66-134 | 36.7 | 7.9e−10 | Ras family |
| 1657 | Hist_deacetyl | 1/1 | 681-1028 | 374.2 | 1.3e−108 | Histone |
| | | | | | | deacetylase |
| | | | | | | family |
| 1658 | RA | 1/1 | 648-735 | 97.5 | 2.6e−25 | Ras association |
| | | | | | | (RalGDS/AF-6) |
| | | | | | | domain |
| 1658 | RasGEF | 1/1 | 240-462 | 194.3 | 1.9e−54 | RasGEF domain |
| 1658 | RasGEFN | 1/1 | 86-140 | 31.4 | 2.4e−07 | Guanine |
| | | | | | | nucleotide |
| | | | | | | exchange factor |
| | | | | | | for Ras-l |
| 1659 | UPF0023 | 1/1 | 5-244 | 332.7 | 4.1e−96 | Uncharacterized |
| | | | | | | protein family |
| | | | | | | UPF0023 |
| 1660 | NNMT_PNMT_TEMT | 1/1 | 1-259 | 593.3 | 1.5e−174 | NNMT/PNMT/TEMT |
| | | | | | | family |
| 1661 | KH | 1/1 | 47-95 | 22.7 | 6e−05 | KH domain |
| 1661 | Ribosomal_S3_C | 1/1 | 104-188 | 112.8 | 2.6e−34 | Ribosomal |
| | | | | | | protein S3, C- |
| | | | | | | terminal domai |
| 1663 | Neurokinin_B | 1/1 | 1-55 | 126.0 | 2.2e−37 | Neurokinin B |
| 1664 | ig | 1/4 | 85-121 | 9.2 | 0.71 | Immunoglobulin |
| | | | | | | domain |
| 1664 | ig | 2/4 | 162-219 | 8.8 | 0.89 | Immunoglobulin |
| | | | | | | domain |
| 1664 | ig | 3/4 | 255-312 | 18.4 | 0.0019 | Immunoglobulin |
| | | | | | | domain |
| 1664 | ig | 4/4 | 347-396 | 30.7 | 7.8e−07 | Immunoglobulin |
| | | | | | | domain |
| 1665 | Ran_BP1 | 1/1 | 37-161 | 306.1 | 4.3e−88 | RanBP1 domain |
| 1666 | bromodomain | 1/1 | 136-223 | 128.7 | 5e−36 | Bromodomain |
| 1668 | WD40 | 1/2 | 171-205 | 12.4 | 0.098 | WD domain, G- |
| | | | | | | beta repeat |
| 1669 | BRICHOS | 1/1 | 68-164 | 144.0 | 1.7e−40 | BRICHOS domain |
| 1670 | Osteopontin | 1/1 | 1-18 | 4.6 | 0.78 | Osteopontin |
| 1670 | TILa | 1/1 | 433-455 | 8.8 | 0.41 | TILa domain |
| 1670 | efhand | 1/1 | 626-654 | 10.9 | 0.4 | EF hand |
| 1670 | kazal | 1/2 | 456-509 | 71.9 | 1.3e−17 | Kazal-type |
| | | | | | | serine protease |
| | | | | | | inhibitor doma |
| 1671 | adh_short | 1/1 | 2-288 | 223.8 | 2.5e−63 | short chain |
| | | | | | | dehydrogenase |
| 1672 | Peptidase_C1 | 1/1 | 114-332 | 442.9 | 5.2e−132 | Papain family |
| | | | | | | cysteine |
| | | | | | | protease |
| 1673 | SNF | 1/1 | 52-597 | 1309.0 | 0 | Sodium: neurotransmitter |
| | | | | | | symporter |
| | | | | | | family |
| 1674 | Gal-bind_lectin | 1/1 | 5-137 | 103.2 | 1e−28 | Galactoside- |
| | | | | | | binding lectin |
| 1675 | helicase_C | 1/1 | 382-477 | 53.7 | 9.4e−14 | Helicase |
| | | | | | | conserved C- |
| | | | | | | terminal domain |
| 1676 | Ribosomal_L7Ae | 1/1 | 16-111 | 143.0 | 2.8e−41 | Ribosomal |
| | | | | | | protein |
| | | | | | | L7Ae/L30e/S12e/ |
| | | | | | | Gadd4 |
| 1677 | GST_C | 1/1 | 83-192 | 106.0 | 1e−28 | Glutathione S- |
| | | | | | | transferase, C- |
| | | | | | | terminal domain |
| 1677 | GST_N | 1/1 | 4-77 | 100.0 | 1.3e−27 | Glutathione S- |
| | | | | | | transferase, N- |
| | | | | | | terminal domain |
| 1678 | Peptidase_S26 | 1/1 | 86-244 | 185.1 | 7.9e−56 | Signal |
| | | | | | | peptidase I |
| 1679 | DNA_pol_delta_4 | 1/1 | 1-107 | 207.1 | 9.2e−63 | DNA polymerase |
| | | | | | | delta, subunit 4 |
| 1680 | Rhomboid | 1/1 | 99-250 | 78.6 | 6.6e−21 | Rhomboid family |
| 1683 | ras | 1/1 | 24-217 | 301.2 | 2.2e−88 | Ras family |
| 1684 | Synaptophysin | 1/1 | 35-260 | 245.2 | 8.9e−70 | Synaptophysin/ |
| | | | | | | synaptoporin |
| 1685 | Pep_M12B_propep | 1/1 | 88-204 | 200.2 | 3.2e−56 | Reprolysin |
| | | | | | | family |
| | | | | | | propeptide |
| 1685 | Reprolysin | 1/1 | 220-414 | 367.1 | 1.9e−106 | Reprolysin |
| | | | | | | (M12B) family |
| | | | | | | zinc metallo |
| 1685 | disintegrin | 1/1 | 431-507 | 82.2 | 3.1e−26 | Disintegrin |
| 1686 | transmembrane4 | 1/1 | 70-316 | 332.6 | 1e−103 | Tetraspanin |
| | | | | | | family |
| 1688 | MBD | 1/1 | 207-260 | 100.1 | 4.5e−26 | Methyl-CpG |
| | | | | | | binding domain |
| 1689 | Sm | 1/1 | 43-122 | 63.7 | 1.2e−16 | Sm protein |
| 1691 | pkinase | 1/2 | 162-383 | 183.1 | 4.5e−51 | Protein kinase |
| | | | | | | domain |
| 1691 | pkinase | 2/2 | 455-482 | 22.9 | 2.9e−05 | Protein kinase |
| | | | | | | domain |

### Example 5

### eMatrix Annotation of Polypeptides of the Invention

Using the eMATRIX software package (Stanford University, Stanford, Calif.) (Wu et al., J. Comp. Biol., vol. 6, pp. 219-235 (1999), herein incorporated by reference), the polypeptides of the invention (SEQ ID NO 853 through 1704) we analyzed. The following table describes the polypeptides of the invention that have significant eMatrix hits:

TABLE 4

| SEQ ID NO: | Domain_ID | Score | p-value | Domain_position | Model_ID | Model_Description |
|---|---|---|---|---|---|---|
| 856 | BL01115A | 10.22 | 6.705e−10 | 7-50 | BL01115 | GTP-binding nuclear |
| | | | | | | protein ran proteins. |
| 856 | BL01115B | 10.81 | 7.308e−09 | 87-130 | BL01115 | GTP-binding nuclear |
| | | | | | | protein ran proteins. |
| 856 | PR00449A | 13.20 | 3.045e−17 | 7-28 | PR00449 | TRANSFORMING PROTEIN |
| | | | | | | P21 RAS SIGNATURE |
| 856 | PR00449B | 14.34 | 6.318e−11 | 30-46 | PR00449 | TRANSFORMING PROTEIN |
| | | | | | | P21 RAS SIGNATURE |
| 856 | PR00449C | 17.27 | 2.895e−18 | 47-69 | PR00449 | TRANSFORMING PROTEIN |
| | | | | | | P21 RAS SIGNATURE |
| 856 | PR00449D | 10.79 | 2.800e−15 | 109-122 | PR00449 | TRANSFORMING PROTEIN |
| | | | | | | P21 RAS SIGNATURE |
| 856 | PR00449E | 13.50 | 9.100e−16 | 157-179 | PR00449 | TRANSFORMING PROTEIN |
| | | | | | | P21 RAS SIGNATURE |
| 857 | BL00222C | 22.97 | 1.333e−26 | 38-65 | BL00222 | Insulin-like growth |
| | | | | | | factor binding |
| | | | | | | proteins. |
| 857 | BL00484B | 9.04 | 2.385e−15 | 39-52 | BL00484 | Thyroglobulin type-1 |
| | | | | | | repeat proteins |
| | | | | | | proteins. |
| 857 | BL00484C | 17.01 | 7.158e−15 | 63-77 | BL00484 | Thyroglobulin type-1 |
| | | | | | | repeat proteins |
| | | | | | | proteins. |
| 860 | BL00194 | 12.16 | 3.348e−13 | 86-98 | BL00194 | Thioredoxin family |
| | | | | | | proteins. |
| 860 | PR00421B | 11.40 | 2.241e−11 | 93-102 | PR00421 | THIOREDOXIN FAMILY |
| | | | | | | SIGNATURE |
| 860 | PR00421C | 13.60 | 5.500e−09 | 136-147 | PR00421 | THIOREDOXIN FAMILY |
| | | | | | | SIGNATURE |
| 861 | BL00288A | 17.47 | 2.059e−36 | 64-93 | BL00288 | Tissue inhibitors of |
| | | | | | | metalloproteinases |
| | | | | | | proteins. |
| 861 | BL00288B | 9.44 | 7.000e−15 | 108-118 | BL00288 | Tissue inhibitors of |
| | | | | | | metalloproteinases |
| | | | | | | proteins. |
| 861 | BL00288C | 14.62 | 6.500e−18 | 127-141 | BL00288 | Tissue inhibitors of |
| | | | | | | metalloproteinases |
| | | | | | | proteins. |
| 861 | BL00288D | 25.76 | 1.000e−40 | 151-195 | BL00288 | Tissue inhibitors of |
| | | | | | | metalloproteinases |
| | | | | | | proteins. |
| 861 | BL00288E | 17.24 | 1.667e−26 | 197-219 | BL00288 | Tissue inhibitors of |
| | | | | | | metalloproteinases |
| | | | | | | proteins. |
| 862 | BL00229A | 23.57 | 9.182e−09 | 216-254 | BL00229 | Tau and MAP proteins |
| | | | | | | tubulin-binding domain |
| | | | | | | proteins. |
| 862 | BL00412D | 16.54 | 3.296e−09 | 175-225 | BL00412 | Neuromodulin (GAP-43) |
| | | | | | | proteins. |
| 862 | BL00412D | 16.54 | 4.398e−09 | 103-153 | BL00412 | Neuromodulin (GAP-43) |
| | | | | | | proteins. |
| 862 | BL00412D | 16.54 | 4.717e−10 | 107-157 | BL00412 | Neuromodulin (GAP-43) |
| | | | | | | proteins. |
| 862 | BL00412D | 16.54 | 5.684e−09 | 128-178 | BL00412 | Neuromodulin (GAP-43) |
| | | | | | | proteins. |
| 862 | BL00412D | 16.54 | 9.724e−09 | 90-140 | BL00412 | Neuromodulin (GAP-43) |
| | | | | | | proteins. |
| 862 | BL00826C | 7.63 | 6.400e−09 | 197-223 | BL00826 | MARCKS family proteins. |
| 863 | BL01033A | 16.94 | 3.250e−20 | 44-65 | BL01033 | Globins profile. |
| 863 | BL01033B | 13.81 | 7.000e−14 | 106-117 | BL01033 | Globins profile. |
| 863 | PR00611A | 15.91 | 5.829e−09 | 44-66 | PR00611 | ERYTHROCRUORIN FAMILY |
| | | | | | | SIGNATURE |
| 863 | PR00612A | 8.64 | 5.800e−14 | 34-46 | PR00612 | ALPHA HAEMOGLOBIN |
| | | | | | | SIGNATURE |
| 863 | PR00612B | 10.92 | 1.000e−13 | 51-61 | PR00612 | ALPHA HAEMOGLOBIN |
| | | | | | | SIGNATURE |
| 863 | PR00612C | 12.16 | 2.286e−13 | 66-75 | PR00612 | ALPHA HAEMOGLOBIN |
| | | | | | | SIGNATURE |
| 863 | PR00612D | 9.76 | 3.400e−15 | 93-106 | PR00612 | ALPHA HAEMOGLOBIN |
| | | | | | | SIGNATURE |
| 863 | PR00612E | 9.04 | 7.000e−19 | 141-157 | PR00612 | ALPHA HAEMOGLOBIN |
| | | | | | | SIGNATURE |
| 864 | BL00077A | 19.44 | 6.478e−34 | 5-45 | BL00077 | Heme-copper oxidase |
| | | | | | | catalytic subunit, |
| | | | | | | copper B binding regio. |
| 864 | BL00077B | 10.15 | 7.750e−36 | 67-112 | BL00077 | Heme-copper oxidase |
| | | | | | | catalytic subunit, |
| | | | | | | copper B binding regio. |
| 864 | BL00077C | 18.98 | 1.000e−40 | 127-177 | BL00077 | Heme-copper oxidase |
| | | | | | | catalytic subunit, |
| | | | | | | copper B binding regio. |
| 864 | BL00077D | 11.17 | 1.000e−40 | 200-254 | BL00077 | Heme-copper oxidase |
| | | | | | | catalytic subunit, |
| | | | | | | copper B binding regio. |
| 864 | BL00077E | 13.71 | 5.179e−38 | 265-313 | BL00077 | Heme-copper oxidase |
| | | | | | | catalytic subunit, |
| | | | | | | copper B binding regio. |
| 864 | BL00077F | 11.35 | 1.000e−40 | 348-398 | BL00077 | Heme-copper oxidase |
| | | | | | | catalytic subunit, |
| | | | | | | copper B binding regio. |
| 864 | BL00077G | 15.93 | 1.000e−40 | 413-461 | BL00077 | Heme-copper oxidase |
| | | | | | | catalytic subunit, |
| | | | | | | copper B binding regio. |
| 866 | BL00175A | 15.42 | 8.333e−20 | 15-34 | BL00175 | Phosphoglycerate mutase |
| | | | | | | family phosphohistidine |
| | | | | | | proteins. |
| 866 | BL00175B | 12.60 | 1.000e−12 | 64-76 | BL00175 | Phosphoglycerate mutase |
| | | | | | | family phosphohistidine |
| | | | | | | proteins. |
| 866 | BL00175C | 23.75 | 5.000e−25 | 88-119 | BL00175 | Phosphoglycerate mutase |
| | | | | | | family phosphohistidine |
| | | | | | | proteins. |
| 866 | BL00175D | 27.67 | 8.500e−40 | 173-224 | BL00175 | Phosphoglycerate mutase |
| | | | | | | family phosphohistidine |
| | | | | | | proteins. |
| 870 | BL00478B | 14.79 | 6.870e−14 | 300-314 | BL00478 | LIM domain proteins. |
| 870 | PD00289 | 9.97 | 6.276e−09 | 72-85 | PD00289 | PROTEIN SH3 DOMAIN |
| | | | | | | REPEAT PRESYNA. |
| 873 | PD01922B | 21.83 | 7.328e−14 | 251-286 | PD01922 | PROTEIN |
| | | | | | | PHOSPHODIESTERASE |
| | | | | | | HYDROL. |
| 874 | BL00030A | 14.39 | 6.294e−11 | 301-319 | BL00030 | Eukaryotic RNA-binding |
| | | | | | | region RNP-1 proteins. |
| 874 | BL00030B | 7.03 | 4.789e−09 | 340-349 | BL00030 | Eukaryotic RNA-binding |
| | | | | | | region RNP-1 proteins. |
| 874 | BL00048 | 6.39 | 1.563e−09 | 122-148 | BL00048 | Protamine P1 proteins. |
| 874 | BL00048 | 6.39 | 1.563e−09 | 123-149 | BL00048 | Protamine P1 proteins. |
| 874 | BL00048 | 6.39 | 1.711e−10 | 106-132 | BL00048 | Protamine P1 proteins. |
| 874 | BL00048 | 6.39 | 1.947e−10 | 117-143 | BL00048 | Protamine P1 proteins. |
| 874 | BL00048 | 6.39 | 2.125e−11 | 120-146 | BL00048 | Protamine P1 proteins. |
| 874 | BL00048 | 6.39 | 3.025e−09 | 121-147 | BL00048 | Protamine P1 proteins. |
| 874 | BL00048 | 6.39 | 3.132e−10 | 127-153 | BL00048 | Protamine P1 proteins. |
| 874 | BL00048 | 6.39 | 3.813e−09 | 110-136 | BL00048 | Protamine P1 proteins. |
| 874 | BL00048 | 6.39 | 4.197e−10 | 118-144 | BL00048 | Protamine P1 proteins. |
| 874 | BL00048 | 6.39 | 5.725e−09 | 108-134 | BL00048 | Protamine P1 proteins. |
| 874 | BL00048 | 6.39 | 6.329e−10 | 114-140 | BL00048 | Protamine P1 proteins. |
| 874 | BL00048 | 6.39 | 6.566e−10 | 116-142 | BL00048 | Protamine P1 proteins. |
| 874 | BL00048 | 6.39 | 6.963e−09 | 125-151 | BL00048 | Protamine P1 proteins. |
| 874 | BL00048 | 6.39 | 7.039e−10 | 119-145 | BL00048 | Protamine P1 proteins. |
| 874 | BL00048 | 6.39 | 7.075e−09 | 115-141 | BL00048 | Protamine P1 proteins. |
| 874 | BL00048 | 6.39 | 7.395e−10 | 112-138 | BL00048 | Protamine P1 proteins. |
| 874 | BL00048 | 6.39 | 7.618e−12 | 124-150 | BL00048 | Protamine P1 proteins. |
| 874 | BL00048 | 6.39 | 7.638e−09 | 126-152 | BL00048 | Protamine P1 proteins. |
| 874 | BL00048 | 6.39 | 9.213e−09 | 113-139 | BL00048 | Protamine P1 proteins. |
| 874 | BL00970C | 14.80 | 9.168e−09 | 99-136 | BL00970 | Nuclear transition |
| | | | | | | protein 2 proteins. |
| 874 | PR00308A | 5.90 | 4.646e−10 | 14-28 | PR00308 | TYPE I ANTIFREEZE |
| | | | | | | PROTEIN SIGNATURE |
| 874 | PR00308A | 5.90 | 4.706e−09 | 16-30 | PR00308 | TYPE I ANTIFREEZE |
| | | | | | | PROTEIN SIGNATURE |
| 874 | PR00308C | 3.83 | 8.831e−09 | 17-26 | PR00308 | TYPE I ANTIFREEZE |
| | | | | | | PROTEIN SIGNATURE |
| 874 | PR00308C | 3.83 | 8.892e−10 | 18-27 | PR00308 | TYPE I ANTIFREEZE |
| | | | | | | PROTEIN SIGNATURE |
| 874 | PR00308C | 3.83 | 8.892e−10 | 19-28 | PR00308 | TYPE I ANTIFREEZE |
| | | | | | | PROTEIN SIGNATURE |
| 874 | PR00308C | 3.83 | 8.892e−10 | 20-29 | PR00308 | TYPE I ANTIFREEZE |
| | | | | | | PROTEIN SIGNATURE |
| 874 | PR00833H | 2.30 | 6.250e−10 | 15-29 | PR00833 | POLLEN ALLERGEN POA PI |
| | | | | | | SIGNATURE |
| 875 | BL00226A | 12.77 | 4.484e−13 | 181-195 | BL00226 | Intermediate filaments |
| | | | | | | proteins. |
| 875 | BL00226B | 23.86 | 7.120e−33 | 282-329 | BL00226 | Intermediate filaments |
| | | | | | | proteins. |
| 875 | BL00226B | 23.86 | 8.744e−09 | 233-280 | BL00226 | Intermediate filaments |
| | | | | | | proteins. |
| 875 | BL00226C | 13.23 | 2.714e−24 | 348-378 | BL00226 | Intermediate filaments |
| | | | | | | proteins. |
| 875 | BL00226D | 19.10 | 2.216e−29 | 451-497 | BL00226 | Intermediate filaments |
| | | | | | | proteins. |
| 875 | BL00970B | 10.09 | 7.033e−09 | 11-36 | BL00970 | Nuclear transition |
| | | | | | | protein 2 proteins. |
| 879 | BL00282 | 16.88 | 3.640e−09 | 478-500 | BL00282 | Kazal serine protease |
| | | | | | | inhibitors family |
| | | | | | | proteins. |
| 880 | BL50058 | 27.23 | 9.654e−33 | 17-64 | BL50058 | G-protein gamma subunit |
| | | | | | | profile. |
| 880 | PR00321A | 11.91 | 6.727e−14 | 16-31 | PR00321 | GAMMA G-PROTEIN |
| | | | | | | (TRANSDUCIN) SIGNATURE |
| 880 | PR00321B | 12.62 | 3.647e−14 | 32-46 | PR00321 | GAMMA G-PROTEIN |
| | | | | | | (TRANSDUCIN) SIGNATURE |
| 880 | PR00321C | 15.39 | 8.816e−16 | 52-69 | PR00321 | GAMMA G-PROTEIN |
| | | | | | | (TRANSDUCIN) SIGNATURE |
| 881 | BL00036 | 9.02 | 3.571e−13 | 331-343 | BL00036 | bZIP transcription |
| | | | | | | factors basic domain |
| | | | | | | proteins. |
| 881 | BL00048 | 6.39 | 2.238e−09 | 6-32 | BL00048 | Protamine P1 proteins. |
| 881 | BL00048 | 6.39 | 5.145e−10 | 5-31 | BL00048 | Protamine P1 proteins. |
| 881 | PR00043A | 9.07 | 1.600e−25 | 309-329 | PR00043 | JUN TRANSCRIPTION |
| | | | | | | FACTOR SIGNATURE |
| 881 | PR00043B | 8.73 | 1.310e−22 | 331-347 | PR00043 | JUN TRANSCRIPTION |
| | | | | | | FACTOR SIGNATURE |
| 881 | PR00043C | 10.55 | 6.824e−16 | 349-361 | PR00043 | JUN TRANSCRIPTION |
| | | | | | | FACTOR SIGNATURE |
| 881 | PR00043D | 11.91 | 5.737e−17 | 364-378 | PR00043 | JUN TRANSCRIPTION |
| | | | | | | FACTOR SIGNATURE |
| 881 | PR00043E | 8.20 | 1.947e−18 | 378-393 | PR00043 | JUN TRANSCRIPTION |
| | | | | | | FACTOR SIGNATURE |
| 881 | PR00308C | 3.83 | 8.013e−09 | 210-219 | PR00308 | TYPE I ANTIFREEZE |
| | | | | | | PROTEIN SIGNATURE |
| 881 | PR00308C | 3.83 | 8.892e−10 | 211-220 | PR00308 | TYPE I ANTIFREEZE |
| | | | | | | PROTEIN SIGNATURE |
| 881 | PR00649B | 8.21 | 9.206e−10 | 212-229 | PR00649 | GPR6 ORPHAN RECEPTOR |
| | | | | | | SIGNATURE |
| 888 | PD00289 | 9.97 | 8.560e−10 | 328-341 | PD00289 | PROTEIN SH3 DOMAIN |
| | | | | | | REPEAT PRESYNA. |
| 888 | PF00424A | 14.34 | 6.266e−09 | 236-267 | PF00424 | REV protein (anti- |
| | | | | | | repression |
| | | | | | | transactivator |
| | | | | | | protein). |
| 889 | BL00226A | 12.77 | 4.960e−14 | 158-172 | BL00226 | Intermediate filaments |
| | | | | | | proteins. |
| 889 | BL00226B | 23.86 | 4.600e−33 | 263-310 | BL00226 | Intermediate filaments |
| | | | | | | proteins. |
| 889 | BL00226C | 13.23 | 8.125e−22 | 328-358 | BL00226 | Intermediate filaments |
| | | | | | | proteins. |
| 889 | BL00226D | 19.10 | 8.054e−29 | 429-475 | BL00226 | Intermediate filaments |
| | | | | | | proteins. |
| 889 | PR00581E | 3.48 | 6.494e−09 | 46-61 | PR00581 | PROSTANOID EP2 RECEPTOR |
| | | | | | | SIGNATURE |
| 891 | BL00406A | 9.95 | 1.000e−40 | 84-118 | BL00406 | Actins proteins. |
| 891 | BL00406B | 5.47 | 1.000e−40 | 160-214 | BL00406 | Actins proteins. |
| 891 | PR00190A | 7.24 | 1.375e−13 | 103-112 | PR00190 | ACTIN SIGNATURE |
| 891 | PR00190B | 9.98 | 1.000e−15 | 126-137 | PR00190 | ACTIN SIGNATURE |
| 891 | PR00190C | 11.49 | 4.789e−30 | 138-160 | PR00190 | ACTIN SIGNATURE |
| 891 | PR00190D | 19.23 | 3.520e−24 | 161-179 | PR00190 | ACTIN SIGNATURE |
| 891 | PR00190E | 7.16 | 1.450e−19 | 192-205 | PR00190 | ACTIN SIGNATURE |
| 891 | PR00190F | 7.80 | 4.349e−21 | 217-236 | PR00190 | ACTIN SIGNATURE |
| 892 | PD02870B | 18.83 | 8.094e−11 | 219-251 | PD02870 | RECEPTOR INTERLEUKIN-1 |
| | | | | | | PRECURSOR. |
| 893 | BL00049A | 13.86 | 8.333e−19 | 32-54 | BL00049 | Ribosomal protein L14 |
| | | | | | | proteins. |
| 893 | BL00049B | 18.42 | 6.806e−24 | 67-98 | BL00049 | Ribosomal protein L14 |
| | | | | | | proteins. |
| 893 | BL00049C | 17.38 | 2.688e−28 | 105-140 | BL00049 | Ribosomal protein L14 |
| | | | | | | proteins. |
| 893 | BL00049D | 13.47 | 5.765e−12 | 142-152 | BL00049 | Ribosomal protein L14 |
| | | | | | | proteins. |
| 894 | BL01221A | 17.26 | 3.739e−27 | 73-100 | BL01221 | PMP-22/EMP/MP20 |
| | | | | | | family proteins. |
| 894 | BL01221B | 13.29 | 7.231e−14 | 108-121 | BL01221 | PMP-22/EMP/MP20 |
| | | | | | | family proteins. |
| 894 | BL01221A | 17.26 | 3.739e−27 | 73-100 | BL01221 | PMP-22/EMP/MP20 |
| | | | | | | family proteins. |
| 894 | BL01221B | 13.29 | 7.231e−14 | 108-121 | BL01221 | PMP-22/EMP/MP20 |
| | | | | | | family proteins. |
| 894 | BL01221C | 26.20 | 8.333e−38 | 131-175 | BL01221 | PMP-22/EMP/MP20 |
| | | | | | | family proteins. |
| 894 | BL01221C | 26.20 | 8.405e−11 | 128-172 | BL01221 | PMP-22/EMP/MP20 |
| | | | | | | family proteins. |
| 894 | BL01221D | 13.99 | 7.577e−28 | 202-228 | BL01221 | PMP-22/EMP/MP20 |
| | | | | | | family proteins. |
| 895 | BL00123A | 10.80 | 6.786e−28 | 77-101 | BL00123 | Alkaline phosphatase |
| | | | | | | proteins. |
| 895 | BL00123B | 19.31 | 1.900e−40 | 115-157 | BL00123 | Alkaline phosphatase |
| | | | | | | proteins. |
| 895 | BL00123C | 24.61 | 1.000e−40 | 170-219 | BL00123 | Alkaline phosphatase |
| | | | | | | proteins. |
| 895 | BL00123D | 12.73 | 5.263e−15 | 241-253 | BL00123 | Alkaline phosphatase |
| | | | | | | proteins. |
| 895 | BL00123E | 22.25 | 1.000e−40 | 329-382 | BL00123 | Alkaline phosphatase |
| | | | | | | proteins. |
| 895 | BL00123F | 19.03 | 6.625e−33 | 389-423 | BL00123 | Alkaline phosphatase |
| | | | | | | proteins. |
| 895 | BL00123G | 26.01 | 1.000e−40 | 463-512 | BL00123 | Alkaline phosphatase |
| | | | | | | proteins. |
| 895 | PR00113A | 11.74 | 9.182e−25 | 77-97 | PR00113 | ALKALINE PHOSPHATASE |
| | | | | | | SIGNATURE |
| 895 | PR00113B | 14.32 | 9.526e−19 | 133-148 | PR00113 | ALKALINE PHOSPHATASE |
| | | | | | | SIGNATURE |
| 895 | PR00113C | 10.88 | 5.846e−25 | 180-200 | PR00113 | ALKALINE PHOSPHATASE |
| | | | | | | SIGNATURE |
| 895 | PR00113D | 6.87 | 6.063e−14 | 239-249 | PR00113 | ALKALINE PHOSPHATASE |
| | | | | | | SIGNATURE |
| 895 | PR00113E | 14.04 | 2.636e−35 | 330-360 | PR00113 | ALKALINE PHOSPHATASE |
| | | | | | | SIGNATURE |
| 898 | BL00236D | 25.66 | 6.860e−19 | 9-50 | BL00236 | Neurotransmitter-gated |
| | | | | | | ion-channels proteins. |
| 898 | PR00253A | 9.15 | 4.667e−15 | 25-45 | PR00253 | GAMMA-AMINOBUTYRIC ACID |
| | | | | | | (GABA) RECEPTOR |
| | | | | | | SIGNATURE |
| 898 | PR00253B | 13.47 | 4.162e−18 | 51-72 | PR00253 | GAMMA-AMINOBUTYRIC ACID |
| | | | | | | (GABA) RECEPTOR |
| | | | | | | SIGNATURE |
| 898 | PR00253C | 13.85 | 3.302e−16 | 85-106 | PR00253 | GAMMA-AMINOBUTYRIC ACID |
| | | | | | | (GABA) RECEPTOR |
| | | | | | | SIGNATURE |
| 898 | PR00253D | 16.68 | 1.000e−18 | 228-248 | PR00253 | GAMMA-AMINOBUTYRIC ACID |
| | | | | | | (GABA) RECEPTOR |
| | | | | | | SIGNATURE |
| 899 | BL00048 | 6.39 | 1.000e−08 | 1924-1950 | BL00048 | Protamine P1 proteins. |
| 899 | BL00048 | 6.39 | 1.132e−12 | 1873-1899 | BL00048 | Protamine P1 proteins. |
| 899 | BL00048 | 6.39 | 1.500e−11 | 1859-1885 | BL00048 | Protamine P1 proteins. |
| 899 | BL00048 | 6.39 | 2.303e−10 | 1996-2022 | BL00048 | Protamine P1 proteins. |
| 899 | BL00048 | 6.39 | 2.421e−10 | 1870-1896 | BL00048 | Protamine P1 proteins. |
| 899 | BL00048 | 6.39 | 2.421e−10 | 1965-1991 | BL00048 | Protamine P1 proteins. |
| 899 | BL00048 | 6.39 | 2.421e−10 | 1972-1998 | BL00048 | Protamine P1 proteins. |
| 899 | BL00048 | 6.39 | 2.456e−12 | 1858-1884 | BL00048 | Protamine P1 proteins. |
| 899 | BL00048 | 6.39 | 2.721e−12 | 1988-2014 | BL00048 | Protamine P1 proteins. |
| 899 | BL00048 | 6.39 | 3.000e−11 | 1866-1892 | BL00048 | Protamine P1 proteins. |
| 899 | BL00048 | 6.39 | 3.132e−10 | 1850-1876 | BL00048 | Protamine P1 proteins. |
| 899 | BL00048 | 6.39 | 3.475e−09 | 1875-1901 | BL00048 | Protamine P1 proteins. |
| 899 | BL00048 | 6.39 | 3.487e−10 | 1857-1883 | BL00048 | Protamine P1 proteins. |
| 899 | BL00048 | 6.39 | 3.605e−10 | 1935-1961 | BL00048 | Protamine P1 proteins. |
| 899 | BL00048 | 6.39 | 3.647e−12 | 1927-1953 | BL00048 | Protamine P1 proteins. |
| 899 | BL00048 | 6.39 | 3.813e−09 | 1855-1881 | BL00048 | Protamine P1 proteins. |
| 899 | BL00048 | 6.39 | 3.875e−11 | 1960-1986 | BL00048 | Protamine P1 proteins. |
| 899 | BL00048 | 6.39 | 3.875e−11 | 1967-1993 | BL00048 | Protamine P1 proteins. |
| 899 | BL00048 | 6.39 | 3.875e−11 | 1974-2000 | BL00048 | Protamine P1 proteins. |
| 899 | BL00048 | 6.39 | 4.044e−12 | 1871-1897 | BL00048 | Protamine P1 proteins. |
| 899 | BL00048 | 6.39 | 4.125e−11 | 1863-1889 | BL00048 | Protamine P1 proteins. |
| 899 | BL00048 | 6.39 | 4.323e−13 | 1860-1886 | BL00048 | Protamine P1 proteins. |
| 899 | BL00048 | 6.39 | 4.375e−09 | 1941-1967 | BL00048 | Protamine P1 proteins. |
| 899 | BL00048 | 6.39 | 4.713e−09 | 1961-1987 | BL00048 | Protamine P1 proteins. |
| 899 | BL00048 | 6.39 | 4.713e−09 | 1968-1994 | BL00048 | Protamine P1 proteins. |
| 899 | BL00048 | 6.39 | 4.713e−09 | 1975-2001 | BL00048 | Protamine P1 proteins. |
| 899 | BL00048 | 6.39 | 4.750e−11 | 1852-1878 | BL00048 | Protamine P1 proteins. |
| 899 | BL00048 | 6.39 | 4.838e−12 | 1861-1887 | BL00048 | Protamine P1 proteins. |
| 899 | BL00048 | 6.39 | 5.103e−12 | 1989-2015 | BL00048 | Protamine P1 proteins. |
| 899 | BL00048 | 6.39 | 5.125e−11 | 1958-1984 | BL00048 | Protamine P1 proteins. |
| 899 | BL00048 | 6.39 | 5.500e−10 | 1867-1893 | BL00048 | Protamine P1 proteins. |
| 899 | BL00048 | 6.39 | 5.500e−12 | 1874-1900 | BL00048 | Protamine P1 proteins. |
| 899 | BL00048 | 6.39 | 5.618e−10 | 1983-2009 | BL00048 | Protamine P1 proteins. |
| 899 | BL00048 | 6.39 | 5.632e−12 | 1981-2007 | BL00048 | Protamine P1 proteins. |
| 899 | BL00048 | 6.39 | 5.737e−10 | 2025-2051 | BL00048 | Protamine P1 proteins. |
| 899 | BL00048 | 6.39 | 5.855e−10 | 1932-1958 | BL00048 | Protamine P1 proteins. |
| 899 | BL00048 | 6.39 | 6.123e−13 | 1862-1888 | BL00048 | Protamine P1 proteins. |
| 899 | BL00048 | 6.39 | 6.125e−11 | 1869-1895 | BL00048 | Protamine P1 proteins. |
| 899 | BL00048 | 6.39 | 6.175e−09 | 1980-2006 | BL00048 | Protamine P1 proteins. |
| 899 | BL00048 | 6.39 | 6.294e−12 | 1853-1879 | BL00048 | Protamine P1 proteins. |
| 899 | BL00048 | 6.39 | 6.294e−12 | 1865-1891 | BL00048 | Protamine P1 proteins. |
| 899 | BL00048 | 6.39 | 6.329e−10 | 1856-1882 | BL00048 | Protamine P1 proteins. |
| 899 | BL00048 | 6.39 | 6.375e−11 | 1986-2012 | BL00048 | Protamine P1 proteins. |
| 899 | BL00048 | 6.39 | 6.500e−11 | 1979-2005 | BL00048 | Protamine P1 proteins. |
| 899 | BL00048 | 6.39 | 6.513e−09 | 1930-1956 | BL00048 | Protamine P1 proteins. |
| 899 | BL00048 | 6.39 | 6.566e−10 | 1951-1977 | BL00048 | Protamine P1 proteins. |
| 899 | BL00048 | 6.39 | 6.684e−10 | 2026-2052 | BL00048 | Protamine P1 proteins. |
| 899 | BL00048 | 6.39 | 6.738e−09 | 2002-2028 | BL00048 | Protamine P1 proteins. |
| 899 | BL00048 | 6.39 | 6.850e−09 | 1993-2019 | BL00048 | Protamine P1 proteins. |
| 899 | BL00048 | 6.39 | 6.875e−11 | 1868-1894 | BL00048 | Protamine P1 proteins. |
| 899 | BL00048 | 6.39 | 6.963e−09 | 1854-1880 | BL00048 | Protamine P1 proteins. |
| 899 | BL00048 | 6.39 | 7.221e−12 | 1982-2008 | BL00048 | Protamine P1 proteins. |
| 899 | BL00048 | 6.39 | 7.231e−13 | 1926-1952 | BL00048 | Protamine P1 proteins. |
| 899 | BL00048 | 6.39 | 7.638e−09 | 1946-1972 | BL00048 | Protamine P1 proteins. |
| 899 | BL00048 | 6.39 | 8.200e−09 | 1950-1976 | BL00048 | Protamine P1 proteins. |
| 899 | BL00048 | 6.39 | 8.375e−11 | 1990-2016 | BL00048 | Protamine P1 proteins. |
| 899 | BL00048 | 6.39 | 8.477e−13 | 1864-1890 | BL00048 | Protamine P1 proteins. |
| 899 | BL00048 | 6.39 | 8.500e−11 | 1928-1954 | BL00048 | Protamine P1 proteins. |
| 899 | BL00048 | 6.39 | 8.544e−12 | 1872-1898 | BL00048 | Protamine P1 proteins. |
| 899 | BL00048 | 6.39 | 8.579e−10 | 1936-1962 | BL00048 | Protamine P1 proteins. |
| 899 | BL00048 | 6.39 | 8.625e−11 | 2021-2047 | BL00048 | Protamine P1 proteins. |
| 899 | BL00048 | 6.39 | 8.875e−09 | 2030-2056 | BL00048 | Protamine P1 proteins. |
| 899 | BL00048 | 6.39 | 9.100e−09 | 1959-1985 | BL00048 | Protamine P1 proteins. |
| 899 | BL00048 | 6.39 | 9.100e−09 | 1966-1992 | BL00048 | Protamine P1 proteins. |
| 899 | BL00048 | 6.39 | 9.100e−09 | 1973-1999 | BL00048 | Protamine P1 proteins. |
| 899 | BL00048 | 6.39 | 9.213e−09 | 1908-1934 | BL00048 | Protamine P1 proteins. |
| 899 | BL00048 | 6.39 | 9.289e−10 | 1848-1874 | BL00048 | Protamine P1 proteins. |
| 899 | BL00048 | 6.39 | 9.289e−10 | 1925-1951 | BL00048 | Protamine P1 proteins. |
| 899 | BL00048 | 6.39 | 9.526e−10 | 2024-2050 | BL00048 | Protamine P1 proteins. |
| 899 | BL00048 | 6.39 | 9.603e−12 | 1851-1877 | BL00048 | Protamine P1 proteins. |
| 899 | BL00048 | 6.39 | 9.625e−11 | 1953-1979 | BL00048 | Protamine P1 proteins. |
| 899 | BL00048 | 6.39 | 9.625e−11 | 1987-2013 | BL00048 | Protamine P1 proteins. |
| 899 | BL00048 | 6.39 | 9.763e−10 | 1849-1875 | BL00048 | Protamine P1 proteins. |
| 899 | BL00048 | 6.39 | 9.775e−09 | 1997-2023 | BL00048 | Protamine P1 proteins. |
| 899 | BL00048 | 6.39 | 9.888e−09 | 1964-1990 | BL00048 | Protamine P1 proteins. |
| 899 | BL00048 | 6.39 | 9.888e−09 | 1971-1997 | BL00048 | Protamine P1 proteins. |
| 899 | BL00048 | 6.39 | 9.888e−09 | 1978-2004 | BL00048 | Protamine P1 proteins. |
| 899 | BL00348F | 23.19 | 8.169e−10 | 103-145 | BL00348 | p53 tumor antigen |
| | | | | | | proteins. |
| 899 | BL00970B | 10.09 | 6.736e−09 | 1952-1977 | BL00970 | Nuclear transition |
| | | | | | | protein 2 proteins. |
| 899 | DM00215 | 19.43 | 4.706e−11 | 1151-1183 | DM00215 | PROLINE-RICH PROTEIN 3. |
| 899 | DM00215 | 19.43 | 6.644e−09 | 1150-1182 | DM00215 | PROLINE-RICH PROTEIN 3. |
| 899 | DM00215 | 19.43 | 8.780e−09 | 1154-1186 | DM00215 | PROLINE-RICH PROTEIN 3. |
| 899 | DM00892C | 23.55 | 7.207e−28 | 2318-2351 | DM00892 | 3 RETROVIRAL |
| | | | | | | PROTEINASE. |
| 899 | DM01206B | 10.69 | 2.139e−09 | 1860-1879 | DM01206 | CORONAVIRUS |
| | | | | | | NUCLEOCAPSID PROTEIN. |
| 900 | BL00284A | 15.64 | 2.565e−18 | 35-58 | BL00284 | Serpins proteins. |
| 900 | BL00284B | 17.99 | 1.621e−13 | 173-193 | BL00284 | Serpins proteins. |
| 900 | BL00284C | 28.56 | 2.038e−29 | 202-243 | BL00284 | Serpins proteins. |
| 900 | BL00284D | 16.34 | 2.241e−17 | 315-341 | BL00284 | Serpins proteins. |
| 900 | BL00284E | 19.15 | 7.158e−21 | 399-423 | BL00284 | Serpins proteins. |
| 901 | BL01160B | 19.54 | 4.203e−09 | 982-1035 | BL01160 | Kinesin light chain |
| | | | | | | repeat proteins. |
| 901 | BL01160B | 19.54 | 8.932e−09 | 1480-1533 | BL01160 | Kinesin light chain |
| | | | | | | repeat proteins. |
| 901 | PR00381A | 9.55 | 1.321e−10 | 969-986 | PR00381 | KINESIN LIGHT CHAIN |
| | | | | | | SIGNATURE |
| 901 | PR00820B | 3.97 | 7.038e−09 | 1132-1146 | PR00820 | CBXX/CFQX PROTEIN |
| | | | | | | SIGNATURE |
| 902 | PR00981A | 11.62 | 4.240e−14 | 336-348 | PR00981 | SERYL-TRNA SYNTHETASE |
| | | | | | | SIGNATURE |
| 902 | PR00981B | 11.68 | 2.552e−15 | 348-361 | PR00981 | SERYL-TRNA SYNTHETASE |
| | | | | | | SIGNATURE |
| 902 | PR00981C | 15.34 | 3.919e−12 | 390-403 | PR00981 | SERYL-TRNA SYNTHETASE |
| | | | | | | SIGNATURE |
| 902 | PR00981D | 18.10 | 1.000e−15 | 407-423 | PR00981 | SERYL-TRNA SYNTHETASE |
| | | | | | | SIGNATURE |
| 902 | PR00981E | 13.40 | 6.667e−20 | 425-441 | PR00981 | SERYL-TRNA SYNTHETASE |
| | | | | | | SIGNATURE |
| 903 | BL00509B | 10.28 | 5.000e−09 | 234-244 | BL00509 | Ras GTPase-activating |
| | | | | | | proteins. |
| 903 | BL01052C | 18.51 | 7.204e−09 | 146-185 | BL01052 | Calponin family repeat |
| | | | | | | proteins. |
| 903 | BL01159 | 13.85 | 5.959e−10 | 650-664 | BL01159 | WW/rsp5/WWP domain |
| | | | | | | proteins. |
| 903 | PR00403B | 12.19 | 3.593e−09 | 650-664 | PR00403 | WW DOMAIN SIGNATURE |
| 904 | BL00385A | 11.67 | 6.727e−39 | 38-69 | BL00385 | Alpha-L-fucosidase |
| | | | | | | proteins. |
| 904 | BL00385B | 24.10 | 1.000e−40 | 79-130 | BL00385 | Alpha-L-fucosidase |
| | | | | | | proteins. |
| 904 | BL00385C | 13.73 | 1.000e−40 | 132-175 | BL00385 | Alpha-L-fucosidase |
| | | | | | | proteins. |
| 904 | BL00385D | 15.93 | 7.923e−29 | 176-199 | BL00385 | Alpha-L-fucosidase |
| | | | | | | proteins. |
| 904 | BL00385E | 16.21 | 1.000e−40 | 213-256 | BL00385 | Alpha-L-fucosidase |
| | | | | | | proteins. |
| 904 | BL00385F | 16.09 | 1.000e−40 | 257-302 | BL00385 | Alpha-L-fucosidase |
| | | | | | | proteins. |
| 904 | BL00385G | 15.69 | 2.800e−36 | 321-354 | BL00385 | Alpha-L-fucosidase |
| | | | | | | proteins. |
| 904 | BL00385H | 11.86 | 7.545e−28 | 355-378 | BL00385 | Alpha-L-fucosidase |
| | | | | | | proteins. |
| 904 | BL00385I | 16.17 | 1.000e−40 | 388-426 | BL00385 | Alpha-L-fucosidase |
| | | | | | | proteins. |
| 904 | PR00741A | 9.24 | 9.308e−20 | 93-108 | PR00741 | GLYCOSYL HYDROLASE |
| | | | | | | FAMILY 29 SIGNATURE |
| 904 | PR00741B | 14.23 | 1.409e−23 | 132-148 | PR00741 | GLYCOSYL HYDROLASE |
| | | | | | | FAMILY 29 SIGNATURE |
| 904 | PR00741C | 9.16 | 1.000e−25 | 151-169 | PR00741 | GLYCOSYL HYDROLASE |
| | | | | | | FAMILY 29 SIGNATURE |
| 904 | PR00741D | 16.11 | 1.621e−25 | 178-197 | PR00741 | GLYCOSYL HYDROLASE |
| | | | | | | FAMILY 29 SIGNATURE |
| 904 | PR00741E | 13.39 | 2.385e−21 | 218-234 | PR00741 | GLYCOSYL HYDROLASE |
| | | | | | | FAMILY 29 SIGNATURE |
| 904 | PR00741F | 14.66 | 8.000e−29 | 246-267 | PR00741 | GLYCOSYL HYDROLASE |
| | | | | | | FAMILY 29 SIGNATURE |
| 904 | PR00741G | 9.29 | 6.870e−26 | 321-342 | PR00741 | GLYCOSYL HYDROLASE |
| | | | | | | FAMILY 29 SIGNATURE |
| 904 | PR00741H | 10.32 | 1.409e−27 | 354-376 | PR00741 | GLYCOSYL HYDROLASE |
| | | | | | | FAMILY 29 SIGNATURE |
| 905 | BL00319C | 17.12 | 6.373e−11 | 371-404 | BL00319 | Amyloidogenic |
| | | | | | | glycoprotein |
| | | | | | | extracellular domain |
| | | | | | | proteins. |
| 905 | BL00319C | 17.12 | 9.171e−09 | 374-407 | BL00319 | Amyloidogenic |
| | | | | | | glycoprotein |
| | | | | | | extracellular domain |
| | | | | | | proteins. |
| 906 | BL00266A | 15.69 | 6.478e−12 | 41-67 | BL00266 | Somatotropin, prolactin |
| | | | | | | and related hormones |
| | | | | | | proteins. |
| 907 | BL00030A | 14.39 | 5.714e−09 | 307-325 | BL00030 | Eukaryotic RNA-binding |
| | | | | | | region RNP-1 proteins. |
| 907 | BL00048 | 6.39 | 1.225e−09 | 461-487 | BL00048 | Protamine P1 proteins. |
| 907 | BL00048 | 6.39 | 1.338e−09 | 452-478 | BL00048 | Protamine P1 proteins. |
| 907 | BL00048 | 6.39 | 1.829e−10 | 457-483 | BL00048 | Protamine P1 proteins. |
| 907 | BL00048 | 6.39 | 2.013e−09 | 552-578 | BL00048 | Protamine P1 proteins. |
| 907 | BL00048 | 6.39 | 2.125e−09 | 474-500 | BL00048 | Protamine P1 proteins. |
| 907 | BL00048 | 6.39 | 2.191e−12 | 454-480 | BL00048 | Protamine P1 proteins. |
| 907 | BL00048 | 6.39 | 2.575e−09 | 455-481 | BL00048 | Protamine P1 proteins. |
| 907 | BL00048 | 6.39 | 2.776e−10 | 450-476 | BL00048 | Protamine P1 proteins. |
| 907 | 9L00048 | 6.39 | 3.842e−10 | 453-479 | BL00048 | Protamine P1 proteins. |
| 907 | 9L00048 | 6.39 | 4.375e−11 | 456-482 | BL00048 | Protamine P1 proteins. |
| 907 | BL00048 | 6.39 | 4.488e−09 | 460-486 | BL00048 | Protamine P1 proteins. |
| 907 | BL00048 | 6.39 | 4.500e−11 | 440-466 | BL00048 | Protamine P1 proteins. |
| 907 | BL00048 | 6.39 | 4.825e−09 | 444-470 | BL00048 | Protamine P1 proteins. |
| 907 | BL00048 | 6.39 | 5.163e−09 | 555-581 | BL00048 | Protamine P1 proteins. |
| 907 | BL00048 | 6.39 | 5.382e−10 | 445-471 | BL00048 | Protamine P1 proteins. |
| 907 | BL00048 | 6.39 | 6.000e−11 | 447-473 | BL00048 | Protamine P1 proteins. |
| 907 | BL00048 | 6.39 | 6.447e−10 | 448-474 | BL00048 | Protamine P1 proteins. |
| 907 | BL00048 | 6.39 | 6.625e−09 | 449-475 | BL00048 | Protamine P1 proteins. |
| 907 | BL00048 | 6.39 | 6.625e−09 | 550-576 | BL00048 | Protamine P1 proteins. |
| 907 | BL00048 | 6.39 | 6.684e−09 | 458-484 | BL00048 | Protamine P1 proteins. |
| 907 | BL00048 | 6.39 | 7.300e−09 | 436-462 | BL00048 | Protamine P1 proteins. |
| 907 | BL00048 | 6.39 | 7.500e−11 | 442-468 | BL00048 | Protamine P1 proteins. |
| 907 | BL00048 | 6.39 | 7.525e−09 | 551-577 | BL00048 | Protamine P1 proteins. |
| 907 | BL00048 | 6.39 | 7.868e−10 | 451-477 | BL00048 | Protamine P1 proteins. |
| 907 | BL00048 | 6.39 | 7.975e−09 | 462-488 | BL00048 | Protamine P1 proteins. |
| 907 | BL00048 | 6.39 | 7.975e−09 | 476-502 | BL00048 | Protamine P1 proteins. |
| 907 | BL00048 | 6.39 | 8.000e−11 | 441-467 | BL00048 | Protamine P1 proteins. |
| 907 | 2L00048 | 6.39 | 8.500e−11 | 459-485 | BL00048 | Protamine P1 proteins. |
| 907 | BL00048 | 6.39 | 8.934e−10 | 438-464 | BL00048 | Protamine P1 proteins. |
| 907 | BL00048 | 6.39 | 9.053e−10 | 446-472 | BL00048 | Protamine P1 proteins. |
| 907 | BL00048 | 6.39 | 9.289e−10 | 443-469 | BL00048 | Protamine P1 proteins. |
| 907 | BL00048 | 6.39 | 9.408e−10 | 439-465 | BL00048 | Protamine P1 proteins. |
| 907 | PF01140D | 15.54 | 6.625e−10 | 559-593 | PF01140 | Matrix protein (MA), |
| | | | | | | p15. |
| 907 | PF01140D | 15.54 | 6.860e−09 | 435-469 | PF01140 | Matrix protein (MA), |
| | | | | | | p15. |
| 909 | BL00450D | 21.14 | 9.534e−09 | 33-56 | BL00450 | Aconitase family |
| | | | | | | proteins. |
| 909 | PR00764F | 16.89 | 9.766e−09 | 51-71 | PR00764 | COMPLEMENT C9 SIGNATURE |
| 910 | BL00048 | 6.39 | 4.938e−09 | 47-73 | BL00048 | Protamine P1 proteins. |
| 911 | BL00292A | 22.87 | 4.115e−25 | 105-138 | BL00292 | Cyclins proteins. |
| 911 | BL00292B | 20.31 | 8.826e−19 | 148-178 | BL00292 | Cyclins proteins. |
| 911 | PD02331A | 19.76 | 8.990e−11 | 121-167 | PD02331 | CYCLIN CELL CYCLE |
| | | | | | | DIVISION PROTE. |
| 912 | BL00888B | 14.79 | 1.310e−13 | 308-331 | BL00888 | Cyclic nucleotide- |
| | | | | | | binding domain |
| | | | | | | proteins. |
| 912 | BL00888B | 14.79 | 7.600e−19 | 190-213 | BL00888 | Cyclic nucleotide- |
| | | | | | | binding domain |
| | | | | | | proteins. |
| 912 | DM01513B | 6.81 | 2.833e−14 | 351-402 | DM01513 | CAMP-DEPENDENT PROTEIN |
| | | | | | | KINASE REGULATORY |
| | | | | | | CHAIN. |
| 912 | DM01513B | 6.81 | 9.871e−34 | 227-278 | DM01513 | CAMP-DEPENDENT PROTEIN |
| | | | | | | KINASE REGULATORY |
| | | | | | | CHAIN. |
| 912 | PR00103A | 9.59 | 3.483e−13 | 305-319 | PR00103 | CAMP-DEPENDENT PROTEIN |
| | | | | | | KINASE SIGNATURE |
| 912 | PR00103A | 9.59 | 5.846e−16 | 187-201 | PR00103 | CAMP-DEPENDENT PROTEIN |
| | | | | | | KINASE SIGNATURE |
| 912 | PR00103B | 13.39 | 5.950e−15 | 202-216 | PR00103 | CAMP-DEPENDENT PROTEIN |
| | | | | | | KINASE SIGNATURE |
| 912 | PR00103B | 13.39 | 7.300e−11 | 320-334 | PR00103 | CAMP-DEPENDENT PROTEIN |
| | | | | | | KINASE SIGNATURE |
| 912 | PR00103C | 15.68 | 2.800e−13 | 351-360 | PR00103 | CAMP-DEPENDENT PROTEIN |
| | | | | | | KINASE SIGNATURE |
| 912 | PR00103D | 10.83 | 2.980e−09 | 239-250 | PR00103 | CAMP-DEPENDENT PROTEIN |
| | | | | | | KINASE SIGNATURE |
| 912 | PR00103D | 10.83 | 3.700e−14 | 363-374 | PR00103 | CAMP-DEPENDENT PROTEIN |
| | | | | | | KINASE SIGNATURE |
| 912 | PR00103E | 17.80 | 2.227e−15 | 384-396 | PR00103 | CAMP-DEPENDENT PROTEIN |
| | | | | | | KINASE SIGNATURE |
| 913 | BL01154A | 18.70 | 3.000e−22 | 57-77 | BL01154 | RNA polymerases L/13 |
| | | | | | | to 16 Kd subunits |
| | | | | | | proteins. |
| 913 | BL01154B | 24.55 | 5.500e−36 | 78-113 | BL01154 | RNA polymerases L/13 |
| | | | | | | to 16 Kd subunits |
| | | | | | | proteins. |
| 914 | BL00048 | 6.39 | 9.100e−09 | 1321-1347 | BL00048 | Protamine P1 proteins. |
| 914 | BL00048 | 6.39 | 9.663e−09 | 1322-1348 | BL00048 | Protamine P1 proteins. |
| 914 | BL00291A | 4.49 | 6.690e−09 | 894-928 | BL00291 | Prion protein. |
| 914 | BL00415C | 7.09 | 3.182e−09 | 450-479 | BL00415 | Synapsins proteins. |
| 914 | BL00420A | 20.42 | 2.938e−09 | 856-884 | BL00420 | Speract receptor repeat |
| | | | | | | proteins domain |
| | | | | | | proteins. |
| 914 | BL00420A | 20.42 | 3.361e−10 | 763-791 | BL00420 | Speract receptor repeat |
| | | | | | | proteins domain |
| | | | | | | proteins. |
| 914 | BL00420A | 20.42 | 3.400e−14 | 1482-1510 | BL00420 | Speract receptor repeat |
| | | | | | | proteins domain |
| | | | | | | proteins. |
| 914 | BL00420A | 20.42 | 3.782e−11 | 617-645 | BL00420 | Speract receptor repeat |
| | | | | | | proteins domain |
| | | | | | | proteins. |
| 914 | BL00420A | 20.42 | 4.246e−10 | 808-836 | BL00420 | Speract receptor repeat |
| | | | | | | proteins domain |
| | | | | | | proteins. |
| 914 | BL00420A | 20.42 | 4.836e−10 | 1485-1513 | BL00420 | Speract receptor repeat |
| | | | | | | proteins domain |
| | | | | | | proteins. |
| 914 | BL00420A | 20.42 | 5.500e−12 | 614-642 | BL00420 | Speract receptor repeat |
| | | | | | | proteins domain |
| | | | | | | proteins. |
| 914 | BL00420A | 20.42 | 6.236e−11 | 679-707 | BL00420 | Speract receptor repeat |
| | | | | | | proteins domain |
| | | | | | | proteins. |
| 914 | BL00420A | 20.42 | 6.400e−09 | 859-887 | BL00420 | Speract receptor repeat |
| | | | | | | proteins domain |
| | | | | | | proteins. |
| 914 | BL00420A | 20.42 | 6.677e−09 | 802-830 | BL00420 | Speract receptor repeat |
| | | | | | | proteins domain |
| | | | | | | proteins. |
| 914 | BL00420A | 20.42 | 7.709e−11 | 620-648 | BL00420 | Speract receptor repeat |
| | | | | | | proteins domain |
| | | | | | | proteins. |
| 914 | BL00420A | 20.42 | 7.923e−09 | 641-669 | BL00420 | Speract receptor repeat |
| | | | | | | proteins domain |
| | | | | | | proteins. |
| 914 | BL00420A | 20.42 | 8.672e−10 | 796-824 | BL00420 | Speract receptor repeat |
| | | | | | | proteins domain |
| | | | | | | proteins. |
| 914 | BL00420A | 20.42 | 9.018e−11 | 644-672 | BL00420 | Speract receptor repeat |
| | | | | | | proteins domain |
| | | | | | | proteins. |
| 914 | BL00420A | 20.42 | 9.031e−09 | 694-722 | BL00420 | Speract receptor repeat |
| | | | | | | proteins domain |
| | | | | | | proteins. |
| 914 | BL00420A | 20.42 | 9.705e−10 | 938-966 | BL00420 | Speract receptor repeat |
| | | | | | | proteins domain |
| | | | | | | proteins. |
| 914 | BL00420A | 20.42 | 9.827e−12 | 721-749 | BL00420 | Speract receptor repeat |
| | | | | | | proteins domain |
| | | | | | | proteins. |
| 914 | BL00420A | 20.42 | 9.836e−11 | 697-725 | BL00420 | Speract receptor repeat |
| | | | | | | proteins domain |
| | | | | | | proteins. |
| 914 | BL01113A | 17.99 | 1.000e−08 | 805-831 | BL01113 | C1q domain proteins. |
| 914 | BL01113A | 17.99 | 1.000e−13 | 802-828 | BL01113 | C1q domain proteins. |
| 914 | BL01113A | 17.99 | 1.173e−09 | 814-840 | BL01113 | C1q domain proteins. |
| 914 | BL01113A | 17.99 | 1.346e−09 | 1028-1054 | BL01113 | C1q domain proteins. |
| 914 | BL01113A | 17.99 | 1.383e−10 | 1244-1270 | BL01113 | C1q domain proteins. |
| 914 | BL01113A | 17.99 | 1.462e−12 | 1473-1499 | BL01113 | C1q domain proteins. |
| 914 | BL01113A | 17.99 | 1.766e−10 | 685-711 | BL01113 | C1q domain proteins. |
| 914 | BL01113A | 17.99 | 2.023e−11 | 617-643 | BL01113 | C1q domain proteins. |
| 914 | BL01113A | 17.99 | 2.038e−09 | 817-843 | BL01113 | C1q domain proteins. |
| 914 | BL01113A | 17.99 | 2.212e−09 | 793-819 | BL01113 | C1q domain proteins. |
| 914 | BL01113A | 17.99 | 2.227e−11 | 947-973 | BL01113 | C1q domain proteins. |
| 914 | BL01113A | 17.99 | 2.385e−09 | 632-658 | BL01113 | C1q domain proteins. |
| 914 | BL01113A | 17.99 | 2.385e−12 | 1491-1517 | BL01113 | C1q domain proteins. |
| 914 | BL01113A | 17.99 | 2.432e−11 | 859-885 | BL01113 | C1q domain proteins. |
| 914 | BL01113A | 17.99 | 2.558e−09 | 620-646 | BL01113 | C1q domain proteins. |
| 914 | BL01113A | 17.99 | 2.731e−09 | 682-708 | BL01113 | C1q domain proteins. |
| 914 | BL01113A | 17.99 | 2.731e−09 | 897-923 | BL01113 | C1q domain proteins. |
| 914 | BL01113A | 17.99 | 3.489e−10 | 1488-1514 | BL01113 | C1q domain proteins. |
| 914 | BL01113A | 17.99 | 3.769e−17 | 856-882 | BL01113 | C1q domain proteins. |
| 914 | BL01113A | 17.99 | 4.068e−11 | 941-967 | BL01113 | C1q domain proteins. |
| 914 | BL01113A | 17.99 | 4.288e−09 | 676-702 | BL01113 | C1q domain proteins. |
| 914 | BL01113A | 17.99 | 4.447e−10 | 862-888 | BL01113 | C1q domain proteins. |
| 914 | BL01113A | 17.99 | 4.462e−09 | 1213-1239 | BL01113 | C1q domain proteins. |
| 914 | BL01113A | 17.99 | 4.462e−09 | 989-1015 | BL01113 | C1q domain proteins. |
| 914 | BL01113A | 17.99 | 4.638e−10 | 811-837 | BL01113 | C1q domain proteins. |
| 914 | BL01113A | 17.99 | 4.981e−09 | 748-774 | BL01113 | C1q domain proteins. |
| 914 | BL01113A | 17.99 | 5.213e−10 | 697-723 | BL01113 | C1q domain proteins. |
| 914 | BL01113A | 17.99 | 5.500e−09 | 611-637 | BL01113 | C1q domain proteins. |
| 914 | BL01113A | 17.99 | 5.500e−11 | 727-753 | BL01113 | C1q domain proteins. |
| 914 | BL01113A | 17.99 | 5.846e−09 | 826-852 | BL01113 | C1q domain proteins. |
| 914 | BL01113A | 17.99 | 5.909e−11 | 944-970 | BL01113 | C1q domain proteins. |
| 914 | BL01113A | 17.99 | 6.365e−09 | 1181-1207 | BL01113 | C1q domain proteins. |
| 914 | BL01113A | 17.99 | 6.365e−09 | 614-640 | BL01113 | C1q domain proteins. |
| 914 | BL01113A | 17.99 | 7.128e−10 | 938-964 | BL01113 | C1q domain proteins. |
| 914 | BL01113A | 17.99 | 7.404e−09 | 820-846 | BL01113 | C1q domain proteins. |
| 914 | BL01113A | 17.99 | 7.404e−09 | 850-876 | BL01113 | C1q domain proteins. |
| 914 | BL01113A | 17.99 | 7.511e−10 | 1497-1523 | BL01113 | C1q domain proteins. |
| 914 | BL01113A | 17.99 | 7.577e−09 | 1253-1279 | BL01113 | C1q domain proteins. |
| 914 | BL01113A | 17.99 | 7.702e−10 | 799-825 | BL01113 | C1q domain proteins. |
| 914 | BL01113A | 17.99 | 7.818e−14 | 623-649 | BL01113 | C1q domain proteins. |
| 914 | BL01113A | 17.99 | 7.955e−11 | 1485-1511 | BL01113 | C1q domain proteins. |
| 914 | BL01113A | 17.99 | 7.955e−11 | 796-822 | BL01113 | C1q domain proteins. |
| 914 | BL01113A | 17.99 | 8.096e−09 | 871-897 | BL01113 | C1q domain proteins. |
| 914 | BL01113A | 17.99 | 8.159e−11 | 808-834 | BL01113 | C1q domain proteins. |
| 914 | BL01113A | 17.99 | 8.364e−11 | 626-652 | BL01113 | C1q domain proteins. |
| 914 | BL01113A | 17.99 | 8.468e−10 | 865-891 | BL01113 | C1q domain proteins. |
| 914 | BL01113A | 17.99 | 8.541e−13 | 1247-1273 | BL01113 | C1q domain proteins. |
| 914 | BL01113A | 17.99 | 8.962e−09 | 1494-1520 | BL01113 | C1q domain proteins. |
| 914 | BL01113A | 17.99 | 8.962e−09 | 691-717 | BL01113 | C1q domain proteins. |
| 914 | BL01113A | 17.99 | 9.308e−09 | 703-729 | BL01113 | C1q domain proteins. |
| 914 | BL01113A | 17.99 | 9.386e−11 | 1482-1508 | BL01113 | C1q domain proteins. |
| 914 | BL01113A | 17.99 | 9.426e−10 | 706-732 | BL01113 | C1q domain proteins. |
| 914 | BL01113A | 17.99 | 9.591e−11 | 853-879 | BL01113 | C1q domain proteins. |
| 914 | BL01113A | 17.99 | 9.617e−10 | 911-937 | BL01113 | C1q domain proteins. |
| 914 | BL01113A | 17.99 | 9.654e−09 | 736-762 | BL01113 | C1q domain proteins. |
| 914 | BL01113A | 17.99 | 9.727e−14 | 986-1012 | BL01113 | C1q domain proteins. |
| 914 | BL01113A | 17.99 | 9.757e−13 | 602-628 | BL01113 | C1q domain proteins. |
| 914 | BL01113A | 17.99 | 9.809e−10 | 739-765 | BL01113 | C1q domain proteins. |
| 914 | PR00049D | 0.00 | 6.339e−09 | 1490-1504 | PR00049 | WILM'S TUMOUR PROTEIN |
| | | | | | | SIGNATURE |
| 914 | PR00049D | 0.00 | 8.322e−09 | 856-870 | PR00049 | WILM'S TUMOUR PROTEIN |
| | | | | | | SIGNATURE |
| 914 | PR00524F | 5.36 | 3.394e−09 | 906-919 | PR00524 | CHOLECYSTOKININ TYPE A |
| | | | | | | RECEPTOR SIGNATURE |
| 914 | PR00524F | 5.36 | 5.552e−10 | 1497-1510 | PR00524 | CHOLECYSTOKININ TYPE A |
| | | | | | | RECEPTOR SIGNATURE |
| 916 | BL00022B | 7.54 | 1.000e−09 | 2640-2646 | BL00022 | EGF-like domain |
| | | | | | | proteins. |
| 916 | BL00022B | 7.54 | 1.000e−09 | 444-450 | BL00022 | EGF-like domain |
| | | | | | | proteins. |
| 916 | BL00022B | 7.54 | 1.900e−09 | 2129-2135 | BL00022 | EGF-like domain |
| | | | | | | proteins. |
| 916 | BL00243H | 17.53 | 4.682e−12 | 253-278 | BL00243 | Integrins beta chain |
| | | | | | | cysteine-rich domain |
| | | | | | | proteins. |
| 916 | BL01177C | 17.39 | 3.000e−09 | 629-647 | BL01177 | Anaphylatoxin domain |
| | | | | | | proteins. |
| 916 | BL01177D | 17.50 | 3.000e−09 | 2097-2114 | BL01177 | Anaphylatoxin domain |
| | | | | | | proteins. |
| 916 | BL01177D | 17.50 | 9.333e−09 | 1321-1338 | BL01177 | Anaphylatoxin domain |
| | | | | | | proteins. |
| 916 | BL01187A | 9.98 | 2.125e−09 | 742-753 | BL01187 | Calcium-binding EGF- |
| | | | | | | like domain proteins |
| | | | | | | pattern proteins. |
| 916 | BL01187A | 9.98 | 2.286e−10 | 1575-1586 | BL01187 | Calcium-binding EGF- |
| | | | | | | like domain proteins |
| | | | | | | pattern proteins. |
| 916 | BL01187A | 9.98 | 2.286e−10 | 2059-2070 | BL01187 | Calcium-binding EGF- |
| | | | | | | like domain proteins |
| | | | | | | pattern proteins. |
| 916 | BL01187A | 9.98 | 2.875e−09 | 375-386 | BL01187 | Calcium-binding EGF- |
| | | | | | | like domain proteins |
| | | | | | | pattern proteins. |
| 916 | BL01187A | 9.98 | 2.895e−11 | 1367-1378 | BL01187 | Calcium-binding EGF- |
| | | | | | | like domain proteins |
| | | | | | | pattern proteins. |
| 916 | BL01187A | 9.98 | 3.455e−14 | 936-947 | BL01187 | Calcium-binding EGF- |
| | | | | | | like domain proteins |
| | | | | | | pattern proteins. |
| 916 | BL01187A | 9.98 | 3.571e−10 | 1492-1503 | BL01187 | Calcium-binding EGF- |
| | | | | | | like domain proteins |
| | | | | | | pattern proteins. |
| 916 | BL01187A | 9.98 | 4.000e−09 | 2142-2153 | BL01187 | Calcium-binding EGF- |
| | | | | | | like domain proteins |
| | | | | | | pattern proteins. |
| 916 | BL01187A | 9.98 | 4.000e−10 | 1039-1050 | BL01187 | Calcium-binding EGF- |
| | | | | | | like domain proteins |
| | | | | | | pattern proteins. |
| 916 | BL01187A | 9.98 | 4.316e−11 | 1937-1948 | BL01187 | Calcium-binding EGF- |
| | | | | | | like domain proteins |
| | | | | | | pattern proteins. |
| 916 | BL01187A | 9.98 | 4.375e−12 | 619-630 | BL01187 | Calcium-binding EGF- |
| | | | | | | like domain proteins |
| | | | | | | pattern proteins. |
| 916 | BL01187A | 9.98 | 4.750e−09 | 1451-1462 | BL01187 | Calcium-binding EGF- |
| | | | | | | like domain proteins |
| | | | | | | pattern proteins. |
| 916 | BL01187A | 9.98 | 5.125e−09 | 1409-1420 | BL01187 | Calcium-binding EGF- |
| | | | | | | like domain proteins |
| | | | | | | pattern proteins. |
| 916 | BL01187A | 9.98 | 5.286e−10 | 417-428 | BL01187 | Calcium-binding EGF- |
| | | | | | | like domain proteins |
| | | | | | | pattern proteins. |
| 916 | BL01187A | 9.98 | 5.500e−09 | 1616-1627 | BL01187 | Calcium-binding EGF- |
| | | | | | | like domain proteins |
| | | | | | | pattern proteins. |
| 916 | BL01187A | 9.98 | 5.714e−10 | 2335-2346 | BL01187 | Calcium-binding EGF- |
| | | | | | | like domain proteins |
| | | | | | | pattern proteins. |
| 916 | BL01187A | 9.98 | 5.875e−09 | 1242-1253 | BL01187 | Calcium-binding EGF- |
| | | | | | | like domain proteins |
| | | | | | | pattern proteins. |
| 916 | BL01187A | 9.98 | 6.250e−09 | 2020-2031 | BL01187 | Calcium-binding EGF- |
| | | | | | | like domain proteins |
| | | | | | | pattern proteins. |
| 916 | BL01187A | 9.98 | 6.625e−09 | 659-670 | BL01187 | Calcium-binding EGF- |
| | | | | | | like domain proteins |
| | | | | | | pattern proteins. |
| 916 | BL01187A | 9.98 | 7.000e−09 | 1284-1295 | BL01187 | Calcium-binding EGF- |
| | | | | | | like domain proteins |
| | | | | | | pattern proteins. |
| 916 | BL01187A | 9.98 | 7.750e−09 | 1777-1788 | BL01187 | Calcium-binding EGF- |
| | | | | | | like domain proteins |
| | | | | | | pattern proteins. |
| 916 | BL01187A | 9.98 | 8.125e−09 | 2295-2306 | BL01187 | Calcium-binding EGF- |
| | | | | | | like domain proteins |
| | | | | | | pattern proteins. |
| 916 | BL01187A | 9.98 | 9.143e−10 | 1199-1210 | BL01187 | Calcium-binding EGF- |
| | | | | | | like domain proteins |
| | | | | | | pattern proteins. |
| 916 | BL01187A | 9.98 | 9.250e−09 | 2614-2625 | BL01187 | Calcium-binding EGF- |
| | | | | | | like domain proteins |
| | | | | | | pattern proteins. |
| 916 | BL01187B | 12.04 | 1.257e−10 | 1592-1607 | BL01187 | Calcium-binding EGF- |
| | | | | | | like domain proteins |
| | | | | | | pattern proteins. |
| 916 | BL01187B | 12.04 | 1.300e−11 | 393-408 | BL01187 | Calcium-binding EGF- |
| | | | | | | like domain proteins |
| | | | | | | pattern proteins. |
| 916 | BL01187B | 12.04 | 1.391e−13 | 435-450 | BL01187 | Calcium-binding EGF- |
| | | | | | | like domain proteins |
| | | | | | | pattern proteins. |
| 916 | BL01187B | 12.04 | 1.450e−14 | 1511-1526 | BL01187 | Calcium-binding EGF- |
| | | | | | | like domain proteins |
| | | | | | | pattern proteins. |
| 916 | BL01187B | 12.04 | 1.667e−12 | 1913-1928 | BL01187 | Calcium-binding EGF- |
| | | | | | | like domain proteins |
| | | | | | | pattern proteins. |
| 916 | BL01187B | 12.04 | 2.000e−12 | 596-611 | BL01187 | Calcium-binding EGF- |
| | | | | | | like domain proteins |
| | | | | | | pattern proteins. |
| 916 | BL01187B | 12.04 | 2.059e−15 | 2273-2288 | BL01187 | Calcium-binding EGF- |
| | | | | | | like domain proteins |
| | | | | | | pattern proteins. |
| 916 | BL01187B | 12.04 | 2.059e−15 | 2631-2646 | BL01187 | Calcium-binding EGF- |
| | | | | | | like domain proteins |
| | | | | | | pattern proteins. |
| 916 | BL01187B | 12.04 | 2.565e−13 | 1427-1442 | BL01187 | Calcium-binding EGF- |
| | | | | | | like domain proteins |
| | | | | | | pattern proteins. |
| 916 | BL01187B | 12.04 | 2.957e−13 | 2160-2175 | BL01187 | Calcium-binding EGF- |
| | | | | | | like domain proteins |
| | | | | | | pattern proteins. |
| 916 | BL01187B | 12.04 | 2.957e−13 | 2672-2687 | BL01187 | Calcium-binding EGF- |
| | | | | | | like domain proteins |
| | | | | | | pattern proteins. |
| 916 | BL01187B | 12.04 | 3.077e−16 | 2590-2605 | BL01187 | Calcium-binding EGF- |
| | | | | | | like domain proteins |
| | | | | | | pattern proteins. |
| 916 | BL01187B | 12.04 | 3.348e−13 | 912-927 | BL01187 | Calcium-binding EGF- |
| | | | | | | like domain proteins |
| | | | | | | pattern proteins. |
| 916 | BL01187B | 12.04 | 3.700e−14 | 1633-1648 | BL01187 | Calcium-binding EGF- |
| | | | | | | like domain proteins |
| | | | | | | pattern proteins. |
| 916 | BL01187B | 12.04 | 4.130e−13 | 1753-1768 | BL01187 | Calcium-binding EGF- |
| | | | | | | like domain proteins |
| | | | | | | pattern proteins. |
| 916 | BL01187B | 12.04 | 4.150e−14 | 1955-1970 | BL01187 | Calcium-binding EGF- |
| | | | | | | like domain proteins |
| | | | | | | pattern proteins. |
| 916 | BL01187B | 12.04 | 4.176e−15 | 2352-2367 | BL01187 | Calcium-binding EGF- |
| | | | | | | like domain proteins |
| | | | | | | pattern proteins. |
| 916 | BL01187B | 12.04 | 4.300e−11 | 870-885 | BL01187 | Calcium-binding EGF- |
| | | | | | | like domain proteins |
| | | | | | | pattern proteins. |
| 916 | BL01187B | 12.04 | 4.333e−12 | 759-774 | BL01187 | Calcium-binding EGF- |
| | | | | | | like domain proteins |
| | | | | | | pattern proteins. |
| 916 | BL01187B | 12.04 | 4.522e−13 | 1343-1358 | BL01187 | Calcium-binding EGF- |
| | | | | | | like domain proteins |
| | | | | | | pattern proteins. |
| 916 | BL01187B | 12.04 | 4.667e−12 | 1217-1232 | BL01187 | Calcium-binding EGF- |
| | | | | | | like domain proteins |
| | | | | | | pattern proteins. |
| 916 | BL01187B | 12.04 | 5.050e−14 | 2438-2453 | BL01187 | Calcium-binding EGF- |
| | | | | | | like domain proteins |
| | | | | | | pattern proteins. |
| 916 | BL01187B | 12.04 | 5.235e−15 | 635-650 | BL01187 | Calcium-binding EGF- |
| | | | | | | like domain proteins |
| | | | | | | pattern proteins. |
| 916 | BL01187B | 12.04 | 5.667e−12 | 1794-1809 | BL01187 | Calcium-binding EGF- |
| | | | | | | like domain proteins |
| | | | | | | pattern proteins. |
| 916 | BL01187B | 12.04 | 6.087e−13 | 1470-1485 | BL01187 | Calcium-binding EGF- |
| | | | | | | like domain proteins |
| | | | | | | pattern proteins. |
| 916 | BL01187B | 12.04 | 6.100e−11 | 1057-1072 | BL01187 | Calcium-binding EGF- |
| | | | | | | like domain proteins |
| | | | | | | pattern proteins. |
| 916 | BL01187B | 12.04 | 6.400e−14 | 2712-2727 | BL01187 | Calcium-binding EGF- |
| | | | | | | like domain proteins |
| | | | | | | pattern proteins. |
| 916 | BL01187B | 12.04 | 6.667e−12 | 1996-2011 | BL01187 | Calcium-binding EGF- |
| | | | | | | like domain proteins |
| | | | | | | pattern proteins. |
| 916 | BL01187B | 12.04 | 6.824e−15 | 2120-2135 | BL01187 | Calcium-binding EGF- |
| | | | | | | like domain proteins |
| | | | | | | pattern proteins. |
| 916 | BL01187B | 12.04 | 6.870e−13 | 2549-2564 | BL01187 | Calcium-binding EGF- |
| | | | | | | like domain proteins |
| | | | | | | pattern proteins. |
| 916 | BL01187B | 12.04 | 7.333e−12 | 2312-2327 | BL01187 | Calcium-binding EGF- |
| | | | | | | like domain proteins |
| | | | | | | pattern proteins. |
| 916 | BL01187B | 12.04 | 7.686e−10 | 1385-1400 | BL01187 | Calcium-binding EGF- |
| | | | | | | like domain proteins |
| | | | | | | pattern proteins. |
| 916 | BL01187B | 12.04 | 7.750e−14 | 2078-2093 | BL01187 | Calcium-binding EGF- |
| | | | | | | like domain proteins |
| | | | | | | pattern proteins. |
| 916 | BL01187B | 12.04 | 7.750e−14 | 2794-2809 | BL01187 | Calcium-binding EGF- |
| | | | | | | like domain proteins |
| | | | | | | pattern proteins. |
| 916 | BL01187B | 12.04 | 8.000e−12 | 1302-1317 | BL01187 | Calcium-binding EGF- |
| | | | | | | like domain proteins |
| | | | | | | pattern proteins. |
| 916 | BL01187B | 12.04 | 8.500e−17 | 2753-2768 | BL01187 | Calcium-binding EGF- |
| | | | | | | like domain proteins |
| | | | | | | pattern proteins. |
| 916 | BL01187B | 12.04 | 8.650e−14 | 677-692 | BL01187 | Calcium-binding EGF- |
| | | | | | | like domain proteins |
| | | | | | | pattern proteins. |
| 916 | BL01187B | 12.04 | 8.667e−12 | 2396-2411 | BL01187 | Calcium-binding EGF- |
| | | | | | | like domain proteins |
| | | | | | | pattern proteins. |
| 916 | BL01187B | 12.04 | 8.826e−13 | 1175-1190 | BL01187 | Calcium-binding EGF- |
| | | | | | | like domain proteins |
| | | | | | | pattern proteins. |
| 916 | BL01187B | 12.04 | 8.826e−13 | 2036-2051 | BL01187 | Calcium-binding EGF- |
| | | | | | | like domain proteins |
| | | | | | | pattern proteins. |
| 916 | BL01187B | 12.04 | 8.826e−13 | 718-733 | BL01187 | Calcium-binding EGF- |
| | | | | | | like domain proteins |
| | | | | | | pattern proteins. |
| 916 | BL01187B | 12.04 | 9.550e−14 | 1260-1275 | BL01187 | Calcium-binding EGF- |
| | | | | | | like domain proteins |
| | | | | | | pattern proteins. |
| 916 | BL01187B | 12.04 | 9.609e−13 | 1551-1566 | BL01187 | Calcium-binding EGF- |
| | | | | | | like domain proteins |
| | | | | | | pattern proteins. |
| 916 | DM00215 | 19.43 | 1.000e−09 | 530-562 | DM00215 | PROLINE-RICH PROTEIN 3. |
| 916 | DM00215 | 19.43 | 6.063e−12 | 545-577 | DM00215 | PROLINE-RICH PROTEIN 3. |
| 916 | DM00864B | 11.34 | 4.896e−10 | 1960-1978 | DM00864 | EGF-LIKE DOMAIN. |
| 916 | DM00864B | 11.34 | 6.577e−09 | 2799-2817 | DM00864 | EGF-LIKE DOMAIN. |
| 916 | DM00864B | 11.34 | 7.179e−10 | 601-619 | DM00864 | EGF-LIKE DOMAIN. |
| 916 | DM00864B | 11.34 | 8.732e−09 | 1556-1574 | DM00864 | EGF-LIKE DOMAIN. |
| 916 | PR00010A | 11.79 | 4.115e−11 | 938-949 | PR00010 | TYPE II EGF-LIKE |
| | | | | | | SIGNATURE |
| 916 | PR00010C | 11.16 | 1.000e−10 | 640-650 | PR00010 | TYPE II EGF-LIKE |
| | | | | | | SIGNATURE |
| 916 | PR00010C | 11.16 | 1.214e−09 | 2443-2453 | PR00010 | TYPE II EGF-LIKE |
| | | | | | | SIGNATURE |
| 916 | PR00010C | 11.16 | 2.286e−09 | 2758-2768 | PR00010 | TYPE II EGF-LIKE |
| | | | | | | SIGNATURE |
| 916 | PR00010C | 11.16 | 2.667e−11 | 1348-1358 | PR00010 | TYPE II EGF-LIKE |
| | | | | | | SIGNATURE |
| 916 | PR00010C | 11.16 | 3.357e−09 | 1918-1928 | PR00010 | TYPE II EGF-LIKE |
| | | | | | | SIGNATURE |
| 916 | PR00010C | 11.16 | 3.455e−10 | 2125-2135 | PR00010 | TYPE II EGF-LIKE |
| | | | | | | SIGNATURE |
| 916 | PR00010C | 11.16 | 3.571e−09 | 2165-2175 | PR00010 | TYPE II EGF-LIKE |
| | | | | | | SIGNATURE |
| 916 | PR00010C | 11.16 | 4.429e−09 | 2717-2727 | PR00010 | TYPE II EGF-LIKE |
| | | | | | | SIGNATURE |
| 916 | PR00010C | 11.16 | 4.818e−10 | 2357-2367 | PR00010 | TYPE II EGF-LIKE |
| | | | | | | SIGNATURE |
| 916 | PR00010C | 11.16 | 5.333e−11 | 917-927 | PR00010 | TYPE II EGF-LIKE |
| | | | | | | SIGNATURE |
| 916 | PR00010C | 11.16 | 5.364e−10 | 440-450 | PR00010 | TYPE II EGF-LIKE |
| | | | | | | SIGNATURE |
| 916 | PR00010C | 11.16 | 5.500e−09 | 2317-2327 | PR00010 | TYPE II EGF-LIKE |
| | | | | | | SIGNATURE |
| 916 | PR00010C | 11.16 | 6.182e−10 | 2677-2687 | PR00010 | TYPE II EGF-LIKE |
| | | | | | | SIGNATURE |
| 916 | PR00010C | 11.16 | 6.357e−09 | 1638-1648 | PR00010 | TYPE II EGF-LIKE |
| | | | | | | SIGNATURE |
| 916 | PR00010C | 11.16 | 7.000e−11 | 2636-2646 | PR00010 | TYPE II EGF-LIKE |
| | | | | | | SIGNATURE |
| 916 | PR00010C | 11.16 | 7.545e−10 | 2595-2605 | PR00010 | TYPE II EGF-LIKE |
| | | | | | | SIGNATURE |
| 916 | PR00010C | 11.16 | 9.143e−09 | 1265-1275 | PR00010 | TYPE II EGF-LIKE |
| | | | | | | SIGNATURE |
| 916 | PR00010C | 11.16 | 9.727e−10 | 2278-2288 | PR00010 | TYPE II EGF-LIKE |
| | | | | | | SIGNATURE |
| 916 | PR00010C | 11.16 | 9.727e−10 | 2799-2809 | PR00010 | TYPE II EGF-LIKE |
| | | | | | | SIGNATURE |
| 916 | PR00907B | 11.29 | 1.000e−08 | 2790-2806 | PR00907 | THROMBOMODULIN |
| | | | | | | SIGNATURE |
| 916 | PR00907B | 11.29 | 2.756e−09 | 2627-2643 | PR00907 | THROMBOMODULIN |
| | | | | | | SIGNATURE |
| 916 | PR00907B | 11.29 | 5.957e−12 | 2708-2724 | PR00907 | THROMBOMODULIN |
| | | | | | | SIGNATURE |
| 916 | PR00907B | 11.29 | 7.805e−09 | 2586-2602 | PR00907 | THROMBOMODULIN |
| | | | | | | SIGNATURE |
| 916 | PR00907D | 5.59 | 8.609e−09 | 2278-2303 | PR00907 | THROMBOMODULIN |
| | | | | | | SIGNATURE |
| 916 | PR00907G | 11.63 | 2.135e−09 | 1592-1618 | PR00907 | THROMBOMODULIN |
| | | | | | | SIGNATURE |
| 916 | PR00907G | 11.63 | 3.189e−09 | 1633-1659 | PR00907 | THROMBOMODULIN |
| | | | | | | SIGNATURE |
| 916 | PR00907G | 11.63 | 3.189e−09 | 2273-2299 | PR00907 | THROMBOMODULIN |
| | | | | | | SIGNATURE |
| 916 | PR00907G | 11.63 | 3.673e−11 | 1260-1286 | PR00907 | THROMBOMODULIN |
| | | | | | | SIGNATURE |
| 916 | PR00907G | 11.63 | 5.546e−12 | 2120-2146 | PR00907 | THROMBOMODULIN |
| | | | | | | SIGNATURE |
| 916 | PR00907G | 11.63 | 6.514e−09 | 718-744 | PR00907 | THROMBOMODULIN |
| | | | | | | SIGNATURE |
| 916 | PR00907G | 11.63 | 8.378e−09 | 1470-1496 | PR00907 | THROMBOMODULIN |
| | | | | | | SIGNATURE |
| 916 | PR00907G | 11.63 | 9.321e−10 | 2396-2422 | PR00907 | THROMBOMODULIN |
| | | | | | | SIGNATURE |
| 916 | PR00907G | 11.63 | 9.406e−10 | 1343-1369 | PR00907 | THROMBOMODULIN |
| | | | | | | SIGNATURE |
| 917 | BL00023 | 24.31 | 2.969e−28 | 332-368 | BL00023 | Type II fibronectin |
| | | | | | | collagen-binding domain |
| | | | | | | proteins. |
| 917 | BL00023 | 24.31 | 4.682e−35 | 390-426 | BL00023 | Type II fibronectin |
| | | | | | | collagen-binding domain |
| | | | | | | proteins. |
| 917 | BL00023 | 24.31 | 9.526e−24 | 274-310 | BL00023 | Type II fibronectin |
| | | | | | | collagen-binding domain |
| | | | | | | proteins. |
| 917 | BL00024A | 11.49 | 2.800e−13 | 145-155 | BL00024 | Hemopexin domain |
| | | | | | | proteins. |
| 917 | BL00024B | 21.53 | 9.591e−33 | 166-199 | BL00024 | Hemopexin domain |
| | | | | | | proteins. |
| 917 | BL00024C | 22.98 | 1.000e−40 | 214-262 | BL00024 | Hemopexin domain |
| | | | | | | proteins. |
| 917 | BL00024D | 17.28 | 3.571e−35 | 442-473 | BL00024 | Hemopexin domain |
| | | | | | | proteins. |
| 917 | BL00024E | 7.58 | 6.625e−16 | 481-494 | BL00024 | Hemopexin domain |
| | | | | | | proteins. |
| 917 | BL00024F | 11.30 | 2.038e−22 | 523-543 | BL00024 | Hemopexin domain |
| | | | | | | proteins. |
| 917 | BL00024G | 13.31 | 7.158e−14 | 561-573 | BL00024 | Hemopexin domain |
| | | | | | | proteins. |
| 917 | BL00024H | 11.35 | 1.794e−11 | 581-592 | BL00024 | Hemopexin domain |
| | | | | | | proteins. |
| 917 | BL00024H | 11.35 | 4.600e−09 | 629-640 | BL00024 | Hemopexin domain |
| | | | | | | proteins. |
| 917 | BL00142 | 8.38 | 6.000e−12 | 448-458 | BL00142 | Neutral zinc |
| | | | | | | metallopeptidases, |
| | | | | | | zinc-binding region |
| | | | | | | proteins. |
| 917 | BL00546A | 19.62 | 8.105e−30 | 125-154 | BL00546 | Matrixins cysteine |
| | | | | | | switch. |
| 917 | BL00546B | 20.11 | 1.000e−40 | 215-258 | BL00546 | Matrixins cysteine |
| | | | | | | switch. |
| 917 | BL00546C | 16.41 | 5.714e−35 | 442-473 | BL00546 | Matrixins cysteine |
| | | | | | | switch. |
| 917 | BL00546D | 10.34 | 4.000e−16 | 481-494 | BL00546 | Matrixins cysteine |
| | | | | | | switch. |
| 917 | BL00546E | 10.23 | 8.269e−23 | 523-543 | BL00546 | Matrixins cysteine |
| | | | | | | switch. |
| 917 | BL00546F | 12.40 | 7.955e−14 | 561-573 | BL00546 | Matrixins cysteine |
| | | | | | | switch. |
| 917 | BL00546G | 16.84 | 8.269e−17 | 582-601 | BL00546 | Matrixins cysteine |
| | | | | | | switch. |
| 917 | BL00546H | 10.76 | 1.257e−10 | 678-688 | BL00546 | Matrixins cysteine |
| | | | | | | switch. |
| 917 | BL00546H | 10.76 | 4.073e−09 | 629-639 | BL00546 | Matrixins cysteine |
| | | | | | | switch. |
| 917 | PR00013A | 12.26 | 5.333e−09 | 336-345 | PR00013 | FIBRONECTIN TYPE II |
| | | | | | | REPEAT SIGNATURE |
| 917 | PR00013A | 12.26 | 5.500e−13 | 394-403 | PR00013 | FIBRONECTIN TYPE II |
| | | | | | | REPEAT SIGNATURE |
| 917 | PR00013A | 12.26 | 7.833e−09 | 278-287 | PR00013 | FIBRONECTIN TYPE II |
| | | | | | | REPEAT SIGNATURE |
| 917 | PR00013B | 14.75 | 1.237e−11 | 405-417 | PR00013 | FIBRONECTIN TYPE II |
| | | | | | | REPEAT SIGNATURE |
| 917 | PR00013B | 14.75 | 4.000e−09 | 347-359 | PR00013 | FIBRONECTIN TYPE II |
| | | | | | | REPEAT SIGNATURE |
| 917 | PR00013C | 12.29 | 1.000e−20 | 422-437 | PR00013 | FIBRONECTIN TYPE II |
| | | | | | | REPEAT SIGNATURE |
| 917 | PR00013C | 12.29 | 3.571e−15 | 364-379 | PR00013 | FIBRONECTIN TYPE II |
| | | | | | | REPEAT SIGNATURE |
| 917 | PR00013C | 12.29 | 7.800e−14 | 306-321 | PR00013 | FIBRONECTIN TYPE II |
| | | | | | | REPEAT SIGNATURE |
| 917 | PR00138A | 15.14 | 8.773e−16 | 145-158 | PR00138 | MATRIXIN SIGNATURE |
| 917 | PR00138B | 15.82 | 5.500e−18 | 192-207 | PR00138 | MATRIXIN SIGNATURE |
| 917 | PR00138C | 16.41 | 1.783e−32 | 215-243 | PR00138 | MATRIXIN SIGNATURE |
| 917 | PR00138D | 16.56 | 8.920e−28 | 448-473 | PR00138 | MATRIXIN SIGNATURE |
| 917 | PR00138E | 6.01 | 5.000e−16 | 481-494 | PR00138 | MATRIXIN SIGNATURE |
| 917 | PR00480B | 15.41 | 2.286e−09 | 443-461 | PR00480 | ASTACIN FAMILY |
| | | | | | | SIGNATURE |
| 918 | PF01130B | 21.40 | 1.537e−15 | 223-268 | PF01130 | CD36 family. |
| 918 | PF01130C | 22.91 | 1.851e−18 | 278-327 | PF01130 | CD36 family. |
| 918 | PF01130D | 26.65 | 1.955e−23 | 360-413 | PF01130 | CD36 family. |
| 920 | BL01289A | 12.18 | 8.200e−33 | 141-167 | BL01289 | TSC-22/dip/bun |
| | | | | | | family proteins. |
| 920 | BL01289B | 10.45 | 8.071e−30 | 168-197 | BL01289 | TSC-22/dip/bun |
| | | | | | | family proteins. |
| 920 | DM00475B | 12.12 | 5.891e−10 | 162-181 | DM00475 | w LOW TRANSPOSASE SAPA |
| | | | | | | 12K. |
| 921 | PD01719A | 12.89 | 2.111e−09 | 416-443 | PD01719 | PRECURSOR GLYCOPROTEIN |
| | | | | | | SIGNAL RE. |
| 921 | PR00711D | 7.91 | 9.697e−09 | 255-272 | PR00711 | ATRIAL NATRIURETIC |
| | | | | | | PEPTIDE SIGNATURE |
| 923 | BL00139A | 10.29 | 6.478e−14 | 107-116 | BL00139 | Eukaryotic thiol |
| | | | | | | (cysteine) proteases |
| | | | | | | cysteine proteins. |
| 923 | BL00139B | 10.19 | 6.400e−10 | 152-160 | BL00139 | Eukaryotic thiol |
| | | | | | | (cysteine) proteases |
| | | | | | | cysteine proteins. |
| 923 | BL00139D | 9.24 | 9.182e−18 | 298-314 | BL00139 | Eukaryotic thiol |
| | | | | | | (cysteine) proteases |
| | | | | | | cysteine proteins. |
| 923 | PR00705A | 10.55 | 1.529e−16 | 107-122 | PR00705 | PAPAIN CYSTEINE |
| | | | | | | PROTEASE (C1) FAMILY |
| | | | | | | SIGNATURE |
| 923 | PR00705B | 10.22 | 4.115e−10 | 283-293 | PR00705 | PAPAIN CYSTEINE |
| | | | | | | PROTEASE (C1) FAMILY |
| | | | | | | SIGNATURE |
| 925 | PF01140D | 15.54 | 6.547e−09 | 176-210 | PF01140 | Matrix protein (MA), |
| | | | | | | p15. |
| 926 | BL00755A | 13.96 | 4.750e−14 | 50-68 | BL00755 | Protein secY proteins. |
| 926 | BL00755B | 12.12 | 7.469e−18 | 87-106 | BL00755 | Protein secY proteins. |
| 926 | BL00755C | 14.21 | 6.885e−13 | 203-218 | BL00755 | Protein secY proteins. |
| 926 | BL00755D | 11.88 | 4.214e−17 | 291-309 | BL00755 | Protein secY proteins. |
| 926 | BL00755E | 26.66 | 1.923e−26 | 385-425 | BL00755 | Protein secY proteins. |
| 926 | PR00303B | 10.17 | 9.769e−09 | 86-106 | PR00303 | PREPROTEIN TRANSLOCASE |
| | | | | | | SECY SUBUNIT SIGNATURE |
| 928 | BL01019A | 13.20 | 1.000e−40 | 69-108 | BL01019 | ADP-ribosylation |
| | | | | | | factors family |
| | | | | | | proteins. |
| 928 | BL01019B | 19.49 | 1.000e−40 | 113-167 | BL01019 | ADP-ribosylation |
| | | | | | | factors family |
| | | | | | | proteins. |
| 928 | BL01019C | 12.52 | 3.842e−26 | 170-195 | BL01019 | ADP-ribosylation |
| | | | | | | factors family |
| | | | | | | proteins. |
| 928 | BL01020C | 15.35 | 8.200e−15 | 97-147 | BL01020 | SAR1 family proteins. |
| 928 | PR00328A | 10.62 | 5.213e−11 | 41-64 | PR00328 | GTP-BINDING SAR1 |
| | | | | | | PROTEIN SIGNATURE |
| 928 | PR00328C | 13.16 | 2.444e−12 | 96-121 | PR00328 | GTP-BINDING SAR1 |
| | | | | | | PROTEIN SIGNATURE |
| 930 | BL00824A | 13.78 | 8.650e−14 | 55-69 | BL00824 | Elongation factor 1 |
| | | | | | | beta/beta′/delta chain |
| | | | | | | proteins. |
| 930 | BL00824B | 9.21 | 2.080e−21 | 132-151 | BL00824 | Elongation factor 1 |
| | | | | | | beta/beta′/delta chain |
| | | | | | | proteins. |
| 930 | BL00824C | 14.58 | 1.000e−40 | 165-202 | BL00824 | Elongation factor 1 |
| | | | | | | beta/beta′/delta chain |
| | | | | | | proteins. |
| 930 | BL00824D | 14.04 | 6.192e−39 | 203-237 | BL00824 | Elongation factor 1 |
| | | | | | | beta/beta′/delta chain |
| | | | | | | proteins. |
| 930 | BL00824E | 12.49 | 3.333e−19 | 246-261 | BL00824 | Elongation factor 1 |
| | | | | | | beta/beta′/delta chain |
| | | | | | | proteins. |
| 931 | BL00291A | 4.49 | 9.192e−11 | 140-174 | BL00291 | Prion protein. |
| 931 | PD01234B | 15.53 | 5.179e−09 | 227-244 | PD01234 | PROTEIN NUCLEAR |
| | | | | | | BROMODOMAIN TRANS. |
| 931 | PR00049D | 0.00 | 7.504e−11 | 85-99 | PR00049 | WILM'S TUMOUR PROTEIN |
| | | | | | | SIGNATURE |
| 931 | PR00049D | 0.00 | 9.214e−10 | 270-284 | PR00049 | WILM'S TUMOUR PROTEIN |
| | | | | | | SIGNATURE |
| 931 | PR00910A | 2.51 | 9.571e−09 | 272-284 | PR00910 | LUTEOVIRUS ORF6 PROTEIN |
| | | | | | | SIGNATURE |
| 933 | BL00415O | 3.44 | 3.368e−09 | 289-326 | BL00415 | Synapsins proteins. |
| 933 | BL01088A | 7.26 | 4.000e−17 | 16-30 | BL01088 | CAP protein. |
| 933 | BL01088B | 15.91 | 1.000e−40 | 150-191 | BL01088 | CAP protein. |
| 933 | BL01088C | 9.97 | 6.684e−26 | 194-216 | BL01088 | CAP protein. |
| 933 | BL01088D | 14.32 | 4.130e−36 | 264-296 | BL01088 | CAP protein. |
| 933 | BL01088E | 15.83 | 1.375e−28 | 326-353 | BL01088 | CAP protein. |
| 933 | BL01088F | 14.83 | 1.000e−40 | 358-403 | BL01088 | CAP protein. |
| 933 | BL01088G | 12.70 | 1.000e−40 | 408-451 | BL01088 | CAP protein. |
| 933 | BL01088H | 12.10 | 7.353e−24 | 453-472 | BL01088 | CAP protein. |
| 933 | PR00049D | 0.00 | 6.143e−11 | 235-249 | PR00049 | WILM'S TUMOUR PROTEIN |
| | | | | | | SIGNATURE |
| 933 | PR00049D | 0.00 | 8.714e−11 | 236-250 | PR00049 | WILM'S TUMOUR PROTEIN |
| | | | | | | SIGNATURE |
| 933 | PR00049D | 0.00 | 8.714e−11 | 237-251 | PR00049 | WILM'S TUMOUR PROTEIN |
| | | | | | | SIGNATURE |
| 933 | PR00049D | 0.00 | 8.790e−11 | 238-252 | PR00049 | WILM'S TUMOUR PROTEIN |
| | | | | | | SIGNATURE |
| 934 | BL00237A | 27.68 | 8.875e−27 | 228-267 | BL00237 | G-protein coupled |
| | | | | | | receptors proteins. |
| 934 | BL00237B | 5.28 | 9.438e−11 | 339-350 | BL00237 | G-protein coupled |
| | | | | | | receptors proteins. |
| 934 | BL00237C | 13.19 | 3.172e−12 | 366-392 | BL00237 | G-protein coupled |
| | | | | | | receptors proteins. |
| 934 | BL00237D | 11.23 | 9.308e−11 | 423-439 | BL00237 | G-protein coupled |
| | | | | | | receptors proteins. |
| 934 | PR00237A | 11.48 | 8.714e−12 | 164-188 | PR00237 | RHODOPSIN-LIKE GPCR |
| | | | | | | SUPERFAMILY SIGNATURE |
| 934 | PR00237B | 13.50 | 4.724e−11 | 197-218 | PR00237 | RHODOPSIN-LIKE GPCR |
| | | | | | | SUPERFAMILY SIGNATURE |
| 934 | PR00237C | 15.69 | 8.800e−18 | 242-264 | PR00237 | RHODOPSIN-LIKE GPCR |
| | | | | | | SUPERFAMILY SIGNATURE |
| 934 | PR00237D | 8.94 | 8.875e−10 | 278-299 | PR00237 | RHODOPSIN-LIKE GPCR |
| | | | | | | SUPERFAMILY SIGNATURE |
| 934 | PR00237E | 13.03 | 7.000e−11 | 331-354 | PR00237 | RHODOPSIN-LIKE GPCR |
| | | | | | | SUPERFAMILY SIGNATURE |
| 934 | PR00237F | 13.57 | 6.516e−11 | 371-395 | PR00237 | RHODOPSIN-LIKE GPCR |
| | | | | | | SUPERFAMILY SIGNATURE |
| 934 | PR00237G | 19.63 | 1.621e−13 | 413-439 | PR00237 | RHODOPSIN-LIKE GPCR |
| | | | | | | SUPERFAMILY SIGNATURE |
| 934 | PR00425C | 13.23 | 3.036e−10 | 228-247 | PR00425 | BRADYKININ RECEPTOR |
| | | | | | | SIGNATURE |
| 934 | PR00571E | 19.50 | 2.333e−09 | 227-238 | PR00571 | ENDOTHELIN-B RECEPTOR |
| | | | | | | SIGNATURE |
| 934 | PR00908A | 7.78 | 5.909e−19 | 65-81 | PR00908 | THROMBIN RECEPTOR |
| | | | | | | SIGNATURE |
| 934 | PR00908B | 16.82 | 2.029e−20 | 148-164 | PR00908 | THROMBIN RECEPTOR |
| | | | | | | SIGNATURE |
| 934 | PR00908C | 9.93 | 1.310e−19 | 190-203 | PR00908 | THROMBIN RECEPTOR |
| | | | | | | SIGNATURE |
| 934 | PR00908D | 11.10 | 4.429e−19 | 269-283 | PR00908 | THROMBIN RECEPTOR |
| | | | | | | SIGNATURE |
| 934 | PR00908E | 3.45 | 1.000e−17 | 326-339 | PR00908 | THROMBIN RECEPTOR |
| | | | | | | SIGNATURE |
| 934 | PR00908F | 8.01 | 1.000e−23 | 355-373 | PR00908 | THROMBIN RECEPTOR |
| | | | | | | SIGNATURE |
| 934 | PR00908G | 13.24 | 5.865e−24 | 438-456 | PR00908 | THROMBIN RECEPTOR |
| | | | | | | SIGNATURE |
| 934 | PR00908H | 11.64 | 1.450e−23 | 467-484 | PR00908 | THROMBIN RECEPTOR |
| | | | | | | SIGNATURE |
| 935 | BL00018 | 7.41 | 1.000e−09 | 79-91 | BL00018 | EF-hand calcium-binding |
| | | | | | | domain proteins. |
| 935 | BL00018 | 7.41 | 3.400e−12 | 152-164 | BL00018 | EF-hand calcium-binding |
| | | | | | | domain proteins. |
| 935 | BL00303B | 26.15 | 6.344e−10 | 67-103 | BL00303 | S-100/ICaBP type |
| | | | | | | calcium binding |
| | | | | | | protein. |
| 935 | PR00450C | 12.22 | 4.480e−10 | 111-132 | PR00450 | RECOVERIN FAMILY |
| | | | | | | SIGNATURE |
| 935 | PR00450C | 12.22 | 5.320e−10 | 38-59 | PR00450 | RECOVERIN FAMILY |
| | | | | | | SIGNATURE |
| 935 | PR00450C | 12.22 | 6.520e−10 | 74-95 | PR00450 | RECOVERIN FAMILY |
| | | | | | | SIGNATURE |
| 936 | BL00030A | 14.39 | 4.500e−10 | 107-125 | BL00030 | Eukaryotic RNA-binding |
| | | | | | | region RNP-1 proteins. |
| 936 | BL00030B | 7.03 | 1.474e−09 | 146-155 | BL00030 | Eukaryotic RNA-binding |
| | | | | | | region RNP-1 proteins. |
| 940 | PR00401B | 12.94 | 3.667e−09 | 129-139 | PR00401 | SH2 DOMAIN SIGNATURE |
| 943 | BL01052A | 16.12 | 7.618e−32 | 35-66 | BL01052 | Calponin family repeat |
| | | | | | | proteins. |
| 943 | BL01052B | 15.31 | 8.031e−26 | 84-109 | BL01052 | Calponin family repeat |
| | | | | | | proteins. |
| 943 | BL01052C | 18.51 | 6.806e−40 | 119-158 | BL01052 | Calponin family repeat |
| | | | | | | proteins. |
| 943 | BL01052D | 10.26 | 1.000e−24 | 206-225 | BL01052 | Calponin family repeat |
| | | | | | | proteins. |
| 943 | PR00888A | 11.87 | 2.350e−16 | 35-49 | PR00888 | SMOOTH MUSCLE |
| | | | | | | PROTEIN/CALPONIN FAMILY |
| | | | | | | SIGNATURE |
| 943 | PR00888B | 13.72 | 2.350e−12 | 54-67 | PR00888 | SMOOTH MUSCLE |
| | | | | | | PROTEIN/CALPONIN FAMILY |
| | | | | | | SIGNATURE |
| 943 | PR00888C | 12.27 | 5.179e−18 | 84-99 | PR00888 | SMOOTH MUSCLE |
| | | | | | | PROTEIN/CALPONIN FAMILY |
| | | | | | | SIGNATURE |
| 943 | PR00888D | 16.09 | 4.273e−17 | 120-136 | PR00888 | SMOOTH MUSCLE |
| | | | | | | PROTEIN/CALPONIN FAMILY |
| | | | | | | SIGNATURE |
| 943 | PR00888E | 11.81 | 3.432e−16 | 136-151 | PR00888 | SMOOTH MUSCLE |
| | | | | | | PROTEIN/CALPONIN FAMILY |
| | | | | | | SIGNATURE |
| 943 | PR00888F | 7.44 | 4.825e−14 | 157-171 | PR00888 | SMOOTH MUSCLE |
| | | | | | | PROTEIN/CALPONIN FAMILY |
| | | | | | | SIGNATURE |
| 943 | PR00888G | 12.73 | 8.759e−14 | 194-207 | PR00888 | SMOOTH MUSCLE |
| | | | | | | PROTEIN/CALPONIN FAMILY |
| | | | | | | SIGNATURE |
| 943 | PR00888H | 9.97 | 5.154e−20 | 207-222 | PR00888 | SMOOTH MUSCLE |
| | | | | | | PROTEIN/CALPONIN FAMILY |
| | | | | | | SIGNATURE |
| 943 | PR00889E | 12.18 | 2.726e−12 | 203-218 | PR00889 | CALPONIN SIGNATURE |
| 943 | PR00890A | 8.61 | 9.775e−21 | 66-85 | PR00890 | SMOOTH MUSCLE PROTEIN |
| | | | | | | 22-ALPHA (TRANSGELIN) |
| | | | | | | SIGNATURE |
| 943 | PR00890B | 8.75 | 3.455e−17 | 94-109 | PR00890 | SMOOTH MUSCLE PROTEIN |
| | | | | | | 22-ALPHA (TRANSGELIN) |
| | | | | | | SIGNATURE |
| 943 | PR00890C | 8.22 | 1.000e−17 | 116-129 | PR00890 | SMOOTH MUSCLE PROTEIN |
| | | | | | | 22-ALPHA (TRANSGELIN) |
| | | | | | | SIGNATURE |
| 943 | PR00890D | 16.17 | 5.174e−13 | 150-159 | PR00890 | SMOOTH MUSCLE PROTEIN |
| | | | | | | 22-ALPHA (TRANSGELIN) |
| | | | | | | SIGNATURE |
| 943 | PR00890E | 14.34 | 3.813e−21 | 167-186 | PR00890 | SMOOTH MUSCLE PROTEIN |
| | | | | | | 22-ALPHA (TRANSGELIN) |
| | | | | | | SIGNATURE |
| 943 | PR00890F | 12.92 | 4.064e−14 | 193-205 | PR00890 | SMOOTH MUSCLE PROTEIN |
| | | | | | | 22-ALPHA (TRANSGELIN) |
| | | | | | | SIGNATURE |
| 947 | BL00415N | 4.29 | 2.248e−09 | 276-319 | BL00415 | Synapsins proteins. |
| 950 | BL01031C | 17.68 | 6.400e−10 | 125-149 | BL01031 | Heat shock hsp20 |
| | | | | | | proteins family |
| | | | | | | profile. |
| 950 | PR00299D | 8.56 | 6.318e−09 | 115-128 | PR00299 | ALPHA CRYSTALLIN |
| | | | | | | SIGNATURE |
| 950 | PR00299F | 13.20 | 2.139e−09 | 152-173 | PR00299 | ALPHA CRYSTALLIN |
| | | | | | | SIGNATURE |
| 951 | BL00019A | 12.56 | 4.789e−15 | 56-66 | BL00019 | Actinin-type actin- |
| | | | | | | binding domain |
| | | | | | | proteins. |
| 951 | BL00019B | 13.34 | 9.250e−23 | 81-103 | BL00019 | Actinin-type actin- |
| | | | | | | binding domain |
| | | | | | | proteins. |
| 951 | BL00019C | 14.66 | 7.300e−37 | 127-162 | BL00019 | Actinin-type actin- |
| | | | | | | binding domain |
| | | | | | | proteins. |
| 951 | BL00019D | 15.33 | 7.000e−33 | 186-215 | BL00019 | Actinin-type actin- |
| | | | | | | binding domain |
| | | | | | | proteins. |
| 951 | BL00019E | 15.91 | 4.000e−13 | 341-350 | BL00019 | Actinin-type actin- |
| | | | | | | binding domain |
| | | | | | | proteins. |
| 951 | BL00019F | 19.68 | 6.464e−19 | 392-413 | BL00019 | Actinin-type actin- |
| | | | | | | binding domain |
| | | | | | | proteins. |
| 951 | BL00303B | 26.15 | 8.172e−10 | 815-851 | BL00303 | S-100/ICaBP type |
| | | | | | | calcium binding |
| | | | | | | protein. |
| 951 | PF00435A | 32.05 | 2.421e−19 | 243-271 | PF00435 | Spectrin repeat |
| | | | | | | proteins. |
| 951 | PF00435B | 13.41 | 2.174e−11 | 508-523 | PF00435 | Spectrin repeat |
| | | | | | | proteins. |
| 951 | PF00435C | 20.73 | 9.036e−09 | 526-545 | PF00435 | Spectrin repeat |
| | | | | | | proteins. |
| 952 | PR00111A | 11.49 | 4.150e−12 | 171-186 | PR00111 | ALPHA/BETA HYDROLASE |
| | | | | | | FOLD SIGNATURE |
| 952 | PR00412A | 13.23 | 6.400e−11 | 151-169 | PR00412 | EPOXIDE HYDROLASE |
| | | | | | | SIGNATURE |
| 952 | PR00412B | 12.59 | 2.929e−10 | 171-186 | PR00412 | EPOXIDE HYDROLASE |
| | | | | | | SIGNATURE |
| 954 | BL01221A | 17.26 | 7.750e−20 | 12-39 | BL01221 | PMP-22/EMP/MP20 |
| | | | | | | family proteins. |
| 954 | BL01221B | 13.29 | 6.182e−12 | 46-59 | BL01221 | PMP-22/EMP/MP20 |
| | | | | | | family proteins. |
| 954 | BL01221C | 26.20 | 8.159e−26 | 69-113 | BL01221 | PMP-22/EMP/MP20 |
| | | | | | | family proteins. |
| 954 | BL01221C | 26.20 | 9.100e−28 | 72-116 | BL01221 | PMP-22/EMP/MP20 |
| | | | | | | family proteins. |
| 955 | PF00783A | 11.91 | 4.214e−13 | 577-586 | PF00783 | Inositol polyphosphate |
| | | | | | | phosphatase, catalytic |
| | | | | | | domain proteins |
| | | | | | | homologu. |
| 955 | PF00783B | 10.54 | 2.552e−10 | 658-667 | PF00783 | Inositol polyphosphate |
| | | | | | | phosphatase, catalytic |
| | | | | | | domain proteins |
| | | | | | | homologu. |
| 955 | PR00049D | 0.00 | 3.071e−10 | 1019-1033 | PR00049 | WILM'S TUMOUR PROTEIN |
| | | | | | | SIGNATURE |
| 955 | PR00049D | 0.00 | 7.407e−09 | 1060-1074 | PR00049 | WILM'S TUMOUR PROTEIN |
| | | | | | | SIGNATURE |
| 956 | BL00030B | 7.03 | 3.368e−09 | 83-92 | BL00030 | Eukaryotic RNA-binding |
| | | | | | | region RNP-1 proteins. |
| 956 | BL00048 | 6.39 | 1.415e−13 | 157-183 | BL00048 | Protamine P1 proteins. |
| 956 | BL00048 | 6.39 | 1.750e−11 | 151-177 | BL00048 | Protamine P1 proteins. |
| 956 | BL00048 | 6.39 | 1.750e−11 | 240-266 | BL00048 | Protamine P1 proteins. |
| 956 | BL00048 | 6.39 | 2.184e−10 | 190-216 | BL00048 | Protamine P1 proteins. |
| 956 | BL00048 | 6.39 | 2.421e−10 | 198-224 | BL00048 | Protamine P1 proteins. |
| 956 | BL00048 | 6.39 | 2.588e−12 | 173-199 | BL00048 | Protamine P1 proteins. |
| 956 | BL00048 | 6.39 | 2.800e−09 | 188-214 | BL00048 | Protamine P1 proteins. |
| 956 | BL00048 | 6.39 | 2.895e−10 | 184-210 | BL00048 | Protamine P1 proteins. |
| 956 | BL00048 | 6.39 | 3.077e−13 | 160-186 | BL00048 | Protamine P1 proteins. |
| 956 | BL00048 | 6.39 | 3.813e−09 | 227-253 | BL00048 | Protamine P1 proteins. |
| 956 | BL00048 | 6.39 | 3.912e−12 | 175-201 | BL00048 | Protamine P1 proteins. |
| 956 | BL00048 | 6.39 | 3.925e−09 | 186-212 | BL00048 | Protamine P1 proteins. |
| 956 | BL00048 | 6.39 | 4.246e−14 | 166-192 | BL00048 | Protamine P1 proteins. |
| 956 | BL00048 | 6.39 | 4.316e−10 | 152-178 | BL00048 | Protamine P1 proteins. |
| 956 | BL00048 | 6.39 | 4.713e−09 | 153-179 | BL00048 | Protamine P1 proteins. |
| 956 | BL00048 | 6.39 | 5.375e−11 | 182-208 | BL00048 | Protamine P1 proteins. |
| 956 | BL00048 | 6.39 | 5.585e−17 | 163-189 | BL00048 | Protamine P1 proteins. |
| 956 | BL00048 | 6.39 | 5.708e−13 | 162-188 | BL00048 | Protamine P1 proteins. |
| 956 | BL00048 | 6.39 | 6.063e−09 | 176-202 | BL00048 | Protamine P1 proteins. |
| 956 | BL00048 | 6.39 | 6.143e−16 | 161-187 | BL00048 | Protamine P1 proteins. |
| 956 | BL00048 | 6.39 | 6.288e−09 | 189-215 | BL00048 | Protamine P1 proteins. |
| 956 | BL00048 | 6.39 | 6.311e−14 | 158-184 | BL00048 | Protamine P1 proteins. |
| 956 | BL00048 | 6.39 | 6.803e−10 | 223-249 | BL00048 | Protamine P1 proteins. |
| 956 | BL00048 | 6.39 | 7.188e−09 | 225-251 | BL00048 | Protamine P1 proteins. |
| 956 | BL00048 | 6.39 | 7.413e−09 | 183-209 | BL00048 | Protamine P1 proteins. |
| 956 | BL00048 | 6.39 | 7.618e−12 | 156-182 | BL00048 | Protamine P1 proteins. |
| 956 | BL00048 | 6.39 | 7.632e−10 | 180-206 | BL00048 | Protamine P1 proteins. |
| 956 | BL00048 | 6.39 | 7.639e−14 | 165-191 | BL00048 | Protamine P1 proteins. |
| 956 | BL00048 | 6.39 | 8.000e−11 | 164-190 | BL00048 | Protamine P1 proteins. |
| 956 | BL00048 | 6.39 | 8.000e−11 | 177-203 | BL00048 | Protamine P1 proteins. |
| 956 | BL00048 | 6.39 | 8.015e−12 | 167-193 | BL00048 | Protamine P1 proteins. |
| 956 | BL00048 | 6.39 | 8.088e−09 | 170-196 | BL00048 | Protamine P1 proteins. |
| 956 | BL00048 | 6.39 | 8.105e−10 | 154-180 | BL00048 | Protamine P1 proteins. |
| 956 | BL00048 | 6.39 | 8.302e−17 | 159-185 | BL00048 | Protamine P1 proteins. |
| 956 | BL00048 | 6.39 | 8.375e−11 | 169-195 | BL00048 | Protamine P1 proteins. |
| 956 | BL00048 | 6.39 | 8.377e−14 | 174-200 | BL00048 | Protamine P1 proteins. |
| 956 | BL00048 | 6.39 | 8.425e−09 | 168-194 | BL00048 | Protamine P1 proteins. |
| 956 | BL00048 | 6.39 | 8.625e−11 | 181-207 | BL00048 | Protamine P1 proteins. |
| 956 | BL00048 | 6.39 | 9.100e−09 | 232-258 | BL00048 | Protamine P1 proteins. |
| 956 | BL00048 | 6.39 | 9.375e−11 | 194-220 | BL00048 | Protamine P1 proteins. |
| 956 | BL00048 | 6.39 | 9.625e−11 | 155-181 | BL00048 | Protamine P1 proteins. |
| 956 | BL00048 | 6.39 | 9.735e−12 | 172-198 | BL00048 | Protamine P1 proteins. |
| 956 | BL00048 | 6.39 | 9.875e−11 | 171-197 | BL00048 | Protamine P1 proteins. |
| 956 | BL00048 | 6.39 | 9.882e−10 | 192-218 | BL00048 | Protamine P1 proteins. |
| 956 | DM01206B | 10.69 | 1.570e−09 | 169-188 | DM01206 | CORONAVIRUS |
| | | | | | | NUCLEOCAPSID PROTEIN. |
| 956 | DM01206B | 10.69 | 4.452e−10 | 163-182 | DM01206 | CORONAVIRUS |
| | | | | | | NUCLEOCAPSID PROTEIN. |
| 956 | DM01206B | 10.69 | 4.532e−09 | 157-176 | DM01206 | CORONAVIRUS |
| | | | | | | NUCLEOCAPSID PROTEIN. |
| 956 | DM01206B | 10.69 | 4.532e−09 | 223-242 | DM01206 | CORONAVIRUS |
| | | | | | | NUCLEOCAPSID PROTEIN. |
| 956 | DM01206B | 10.69 | 5.899e−09 | 155-174 | DM01206 | CORONAVIRUS |
| | | | | | | NUCLEOCAPSID PROTEIN. |
| 956 | DM01206B | 10.69 | 7.411e−10 | 159-178 | DM01206 | CORONAVIRUS |
| | | | | | | NUCLEOCAPSID PROTEIN. |
| 956 | PD02784B | 26.46 | 9.745e−09 | 79-121 | PD02784 | PROTEIN NUCLEAR |
| | | | | | | RIBONUCLEOPROTEIN. |
| 957 | BL01160B | 19.54 | 5.653e−09 | 253-306 | BL01160 | Kinesin light chain |
| | | | | | | repeat proteins. |
| 957 | BL01160B | 19.54 | 5.710e−11 | 260-313 | BL01160 | Kinesin light chain |
| | | | | | | repeat proteins. |
| 958 | PR00049D | 0.00 | 6.445e−11 | 430-444 | PR00049 | WILM'S TUMOUR PROTEIN |
| | | | | | | SIGNATURE |
| 959 | BL00250A | 21.24 | 7.000e−35 | 340-375 | BL00250 | TGF-beta family |
| | | | | | | proteins. |
| 959 | BL00250B | 27.37 | 8.071e−32 | 409-444 | BL00250 | TGF-beta family |
| | | | | | | proteins. |
| 959 | PR00438A | 13.54 | 7.300e−11 | 365-374 | PR00438 | GROWTH FACTOR CYSTINE |
| | | | | | | KNOT SUPERFAMILY |
| | | | | | | SIGNATURE |
| 959 | PR00669F | 5.57 | 9.494e−09 | 357-374 | PR00669 | INHIBIN ALPHA CHAIN |
| | | | | | | SIGNATURE |
| 959 | PR00670A | 11.24 | 1.750e−24 | 21-38 | PR00670 | INHIBIN BETA A CHAIN |
| | | | | | | SIGNATURE |
| 959 | PR00670B | 13.06 | 1.360e−28 | 38-59 | PR00670 | INHIBIN BETA A CHAIN |
| | | | | | | SIGNATURE |
| 959 | PR00670C | 11.98 | 3.250e−19 | 83-96 | PR00670 | INHIBIN BETA A CHAIN |
| | | | | | | SIGNATURE |
| 959 | PR00670D | 13.39 | 1.231e−22 | 123-139 | PR00670 | INHIBIN BETA A CHAIN |
| | | | | | | SIGNATURE |
| 959 | PR00670E | 3.63 | 1.333e−25 | 268-289 | PR00670 | INHIBIN BETA A CHAIN |
| | | | | | | SIGNATURE |
| 959 | PR00670F | 10.10 | 4.818e−13 | 298-308 | PR00670 | INHIBIN BETA A CHAIN |
| | | | | | | SIGNATURE |
| 960 | BL00154C | 12.38 | 6.580e−12 | 79-97 | BL00154 | E1-E2 ATPases |
| | | | | | | phosphorylation site |
| | | | | | | proteins. |
| 960 | BL00154E | 20.37 | 9.820e−15 | 347-387 | BL00154 | E1-E2 ATPases |
| | | | | | | phosphorylation site |
| | | | | | | proteins. |
| 960 | BL00154F | 8.23 | 8.859e−10 | 502-525 | BL00154 | E1-E2 ATPases |
| | | | | | | phosphorylation site |
| | | | | | | proteins. |
| 960 | BL00592E | 12.53 | 9.100e−09 | 168-184 | BL00592 | Glycosyl hydrolases |
| | | | | | | family 9 proteins. |
| 960 | BL01228D | 17.44 | 7.150e−11 | 503-527 | BL01228 | Hypothetical cof family |
| | | | | | | proteins. |
| 960 | PR00119B | 13.94 | 6.694e−12 | 83-97 | PR00119 | P-TYPE CATION- |
| | | | | | | TRANSPORTING ATPASE |
| | | | | | | SUPERFAMILY SIGNATURE |
| 960 | PR00119D | 9.56 | 2.432e−09 | 363-373 | PR00119 | P-TYPE CATION- |
| | | | | | | TRANSPORTING ATPASE |
| | | | | | | SUPERFAMILY SIGNATURE |
| 960 | PR00121D | 16.72 | 4.621e−11 | 76-97 | PR00121 | SODIUM/POTASSIUM- |
| | | | | | | TRANSPORTING ATPASE |
| | | | | | | SIGNATURE |
| 961 | BL00030A | 14.39 | 5.765e−11 | 14-32 | BL00030 | Eukaryotic RNA-binding |
| | | | | | | region RNP-1 proteins. |
| 961 | BL00030A | 14.39 | 6.571e−09 | 297-315 | BL00030 | Eukaryotic RNA-binding |
| | | | | | | region RNP-1 proteins. |
| 961 | BL00030B | 7.03 | 4.316e−09 | 334-343 | BL00030 | Eukaryotic RNA-binding |
| | | | | | | region RNP-1 proteins. |
| 961 | BL00030B | 7.03 | 6.400e−10 | 232-241 | BL00030 | Eukaryotic RNA-binding |
| | | | | | | region RNP-1 proteins. |
| 961 | BL00030B | 7.03 | 8.579e−09 | 139-148 | BL00030 | Eukaryotic RNA-binding |
| | | | | | | region RNP-1 proteins. |
| 961 | PD02784A | 21.09 | 4.583e−09 | 94-130 | PD02784 | PROTEIN NUCLEAR |
| | | | | | | RIBONUCLEOPROTEIN. |
| 961 | PD02784B | 26.46 | 4.651e−09 | 49-91 | PD02784 | PROTEIN NUCLEAR |
| | | | | | | RIBONUCLEOPROTEIN. |
| 961 | PF00658A | 19.88 | 1.000e−40 | 84-123 | PF00658 | Poly-adenylate binding |
| | | | | | | protein, unique domain |
| | | | | | | proteins. |
| 961 | PF00658B | 28.57 | 1.000e−40 | 263-309 | PF00658 | Poly-adenylate binding |
| | | | | | | protein, unique domain |
| | | | | | | proteins. |
| 961 | PF00658C | 16.33 | 8.500e−36 | 579-615 | PF00658 | Poly-adenylate binding |
| | | | | | | protein, unique domain |
| | | | | | | proteins. |
| 962 | BL00036 | 9.02 | 5.737e−11 | 682-694 | BL00036 | bZIP transcription |
| | | | | | | factors basic domain |
| | | | | | | proteins. |
| 962 | PF00624I | 9.10 | 4.331e−09 | 507-536 | PF00624 | Flocculin repeat |
| | | | | | | proteins. |
| 962 | PF00624I | 9.10 | 5.125e−10 | 510-539 | PF00624 | Flocculin repeat |
| | | | | | | proteins. |
| 962 | PF00624I | 9.10 | 5.800e−10 | 511-540 | PF00624 | Flocculin repeat |
| | | | | | | proteins. |
| 962 | PF00624I | 9.10 | 6.457e−09 | 505-534 | PF00624 | Flocculin repeat |
| | | | | | | proteins. |
| 962 | PF00624I | 9.10 | 6.811e−09 | 502-531 | PF00624 | Flocculin repeat |
| | | | | | | proteins. |
| 962 | PF00624I | 9.10 | 8.441e−09 | 503-532 | PF00624 | Flocculin repeat |
| | | | | | | proteins. |
| 962 | PR00043B | 8.73 | 9.241e−11 | 682-698 | PR00043 | JUN TRANSCRIPTION |
| | | | | | | FACTOR SIGNATURE |
| 963 | BL01160B | 19.54 | 9.676e−10 | 308-361 | BL01160 | Kinesin light chain |
| | | | | | | repeat proteins. |
| 964 | BL00291A | 4.49 | 1.000e−09 | 540-574 | BL00291 | Prion protein. |
| 964 | BL00415N | 4.29 | 8.095e−09 | 347-390 | BL00415 | Synapsins proteins. |
| 964 | BL00420A | 20.42 | 1.000e−12 | 345-373 | BL00420 | Speract receptor repeat |
| | | | | | | proteins domain |
| | | | | | | proteins. |
| 964 | BL00420A | 20.42 | 1.148e−10 | 810-838 | BL00420 | Speract receptor repeat |
| | | | | | | proteins domain |
| | | | | | | proteins. |
| 964 | BL00420A | 20.42 | 1.295e−10 | 873-901 | BL00420 | Speract receptor repeat |
| | | | | | | proteins domain |
| | | | | | | proteins. |
| 964 | BL00420A | 20.42 | 1.590e−10 | 840-868 | BL00420 | Speract receptor repeat |
| | | | | | | proteins domain |
| | | | | | | proteins. |
| 964 | BL00420A | 20.42 | 1.692e−09 | 438-466 | BL00420 | Speract receptor repeat |
| | | | | | | proteins domain |
| | | | | | | proteins. |
| 964 | BL00420A | 20.42 | 1.738e−10 | 459-487 | BL00420 | Speract receptor repeat |
| | | | | | | proteins domain |
| 964 | BL00420A | 20.42 | 1.885e−10 | 207-235 | BL00420 | Speract receptor repeat |
| | | | | | | proteins domain |
| | | | | | | proteins. |
| 964 | BL00420A | 20.42 | 1.969e−09 | 204-232 | BL00420 | Speract receptor repeat |
| | | | | | | proteins domain |
| | | | | | | proteins. |
| 964 | BL00420A | 20.42 | 2.108e−09 | 198-226 | BL00420 | Speract receptor repeat |
| | | | | | | proteins domain |
| | | | | | | proteins. |
| 964 | BL00420A | 20.42 | 2.108e−09 | 747-775 | BL00420 | Speract receptor repeat |
| | | | | | | proteins domain |
| | | | | | | proteins. |
| 964 | BL00420A | 20.42 | 2.108e−09 | 867-895 | BL00420 | Speract receptor repeat |
| | | | | | | proteins domain |
| | | | | | | proteins. |
| 964 | BL00420A | 20.42 | 2.246e−09 | 216-244 | BL00420 | Speract receptor repeat |
| | | | | | | proteins domain |
| | | | | | | proteins. |
| 964 | BL00420A | 20.42 | 2.246e−09 | 669-697 | BL00420 | Speract receptor repeat |
| | | | | | | proteins domain |
| | | | | | | proteins. |
| 964 | BL00420A | 20.42 | 2.523e−09 | 642-670 | BL00420 | Speract receptor repeat |
| | | | | | | proteins domain |
| | | | | | | proteins. |
| 964 | BL00420A | 20.42 | 2.800e−09 | 219-247 | BL00420 | Speract receptor repeat |
| | | | | | | proteins domain |
| | | | | | | proteins. |
| 964 | BL00420A | 20.42 | 2.800e−09 | 516-544 | BL00420 | Speract receptor repeat |
| | | | | | | proteins domain |
| | | | | | | proteins. |
| 964 | BL00420A | 20.42 | 2.800e−09 | 564-592 | BL00420 | Speract receptor repeat |
| | | | | | | proteins domain |
| | | | | | | proteins. |
| 964 | BL00420A | 20.42 | 3.066e−10 | 798-826 | BL00420 | Speract receptor repeat |
| | | | | | | proteins domain |
| | | | | | | proteins. |
| 964 | BL00420A | 20.42 | 3.077e−09 | 687-715 | BL00420 | Speract receptor repeat |
| | | | | | | proteins domain |
| | | | | | | proteins. |
| 964 | BL00420A | 20.42 | 3.213e−10 | 552-580 | BL00420 | Speract receptor repeat |
| | | | | | | proteins domain |
| | | | | | | proteins. |
| 964 | BL00420A | 20.42 | 3.215e−09 | 330-358 | BL00420 | Speract receptor repeat |
| | | | | | | proteins domain |
| | | | | | | proteins. |
| 964 | BL00420A | 20.42 | 3.354e−09 | 588-616 | BL00420 | Speract receptor repeat |
| | | | | | | proteins domain |
| | | | | | | proteins. |
| 964 | BL00420A | 20.42 | 3.361e−10 | 627-655 | BL00420 | Speract receptor repeat |
| | | | | | | proteins domain |
| | | | | | | proteins. |
| 964 | BL00420A | 20.42 | 3.492e−09 | 240-268 | BL00420 | Speract receptor repeat |
| | | | | | | proteins domain |
| | | | | | | proteins. |
| 964 | BL00420A | 20.42 | 3.508e−10 | 327-355 | BL00420 | Speract receptor repeat |
| | | | | | | proteins domain |
| | | | | | | proteins. |
| 964 | BL00420A | 20.42 | 3.618e−11 | 807-835 | BL00420 | Speract receptor repeat |
| | | | | | | proteins domain |
| | | | | | | proteins. |
| 964 | BL00420A | 20.42 | 3.631e−09 | 106-134 | BL00420 | Speract receptor repeat |
| | | | | | | proteins domain |
| | | | | | | proteins. |
| 964 | BL00420A | 20.42 | 3.631e−09 | 339-367 | BL00420 | Speract receptor repeat |
| | | | | | | proteins domain |
| | | | | | | proteins. |
| 964 | BL00420A | 20.42 | 4.098e−10 | 366-394 | BL00420 | Speract receptor repeat |
| | | | | | | proteins domain |
| | | | | | | proteins. |
| 964 | BL00420A | 20.42 | 4.098e−10 | 675-703 | BL00420 | Speract receptor repeat |
| | | | | | | proteins domain |
| | | | | | | proteins. |
| 964 | BL00420A | 20.42 | 4.185e−09 | 348-376 | BL00420 | Speract receptor repeat |
| | | | | | | proteins domain |
| | | | | | | proteins. |
| 964 | BL00420A | 20.42 | 4.185e−09 | 486-514 | BL00420 | Speract receptor repeat |
| | | | | | | proteins domain |
| | | | | | | proteins. |
| 964 | BL00420A | 20.42 | 4.185e−09 | 756-784 | BL00420 | Speract receptor repeat |
| | | | | | | proteins domain |
| | | | | | | proteins. |
| 964 | BL00420A | 20.42 | 4.306e−13 | 720-748 | BL00420 | Speract receptor repeat |
| | | | | | | proteins domain |
| | | | | | | proteins. |
| 964 | BL00420A | 20.42 | 4.689e−10 | 480-508 | BL00420 | Speract receptor repeat |
| | | | | | | proteins domain |
| | | | | | | proteins. |
| 964 | BL00420A | 20.42 | 4.689e−10 | 570-598 | BL00420 | Speract receptor repeat |
| | | | | | | proteins domain |
| | | | | | | proteins. |
| 964 | BL00420A | 20.42 | 4.836e−10 | 195-223 | BL00420 | Speract receptor repeat |
| | | | | | | proteins domain |
| | | | | | | proteins. |
| 964 | BL00420A | 20.42 | 4.927e−11 | 705-733 | BL00420 | Speract receptor repeat |
| | | | | | | proteins domain |
| | | | | | | proteins. |
| 964 | BL00420A | 20.42 | 5.015e−09 | 189-217 | BL00420 | Speract receptor repeat |
| | | | | | | proteins domain |
| | | | | | | proteins. |
| 964 | BL00420A | 20.42 | 5.091e−11 | 180-208 | BL00420 | Speract receptor repeat |
| | | | | | | proteins domain |
| | | | | | | proteins. |
| 964 | BL00420A | 20.42 | 5.154e−09 | 861-889 | BL00420 | Speract receptor repeat |
| | | | | | | proteins domain |
| | | | | | | proteins. |
| 964 | BL00420A | 20.42 | 5.426e−10 | 774-802 | BL00420 | Speract receptor repeat |
| | | | | | | proteins domain |
| | | | | | | proteins. |
| 964 | BL00420A | 20.42 | 5.600e−14 | 408-436 | BL00420 | Speract receptor repeat |
| | | | | | | proteins domain |
| | | | | | | proteins. |
| 964 | BL00420A | 20.42 | 5.708e−09 | 372-400 | BL00420 | Speract receptor repeat |
| | | | | | | proteins domain |
| | | | | | | proteins. |
| 964 | BL00420A | 20.42 | 5.721e−10 | 651-679 | BL00420 | Speract receptor repeat |
| | | | | | | proteins domain |
| | | | | | | proteins. |
| 964 | BL00420A | 20.42 | 5.776e−13 | 426-454 | BL00420 | Speract receptor repeat |
| | | | | | | proteins domain |
| | | | | | | proteins. |
| 964 | BL00420A | 20.42 | 5.846e−09 | 300-328 | BL00420 | Speract receptor repeat |
| | | | | | | proteins domain |
| | | | | | | proteins. |
| 964 | BL00420A | 20.42 | 5.869e−10 | 109-137 | BL00420 | Speract receptor repeat |
| | | | | | | proteins domain |
| | | | | | | proteins. |
| 964 | BL00420A | 20.42 | 6.016e−10 | 537-565 | BL00420 | Speract receptor repeat |
| | | | | | | proteins domain |
| | | | | | | proteins. |
| 964 | BL00420A | 20.42 | 6.073e−11 | 801-829 | BL00420 | Speract receptor repeat |
| | | | | | | proteins domain |
| | | | | | | proteins. |
| 964 | BL00420A | 20.42 | 6.123e−09 | 546-574 | BL00420 | Speract receptor repeat |
| | | | | | | proteins domain |
| | | | | | | proteins. |
| 964 | BL00420A | 20.42 | 6.123e−09 | 603-631 | BL00420 | Speract receptor repeat |
| | | | | | | proteins domain |
| | | | | | | proteins. |
| 964 | BL00420A | 20.42 | 6.164e−10 | 306-334 | BL00420 | Speract receptor repeat |
| | | | | | | proteins domain |
| | | | | | | proteins. |
| 964 | BL00420A | 20.42 | 6.164e−10 | 483-511 | BL00420 | Speract receptor repeat |
| | | | | | | proteins domain |
| | | | | | | proteins. |
| 964 | BL00420A | 20.42 | 6.164e−10 | 549-577 | BL00420 | Speract receptor repeat |
| | | | | | | proteins domain |
| | | | | | | proteins. |
| 964 | BL00420A | 20.42 | 6.164e−10 | 777-805 | BL00420 | Speract receptor repeat |
| | | | | | | proteins domain |
| | | | | | | proteins. |
| 964 | BL00420A | 20.42 | 6.262e−09 | 183-211 | BL00420 | Speract receptor repeat |
| | | | | | | proteins domain |
| | | | | | | proteins. |
| 964 | BL00420A | 20.42 | 6.262e−09 | 837-865 | BL00420 | Speract receptor repeat |
| | | | | | | proteins domain |
| | | | | | | proteins. |
| 964 | BL00420A | 20.42 | 6.400e−09 | 741-769 | BL00420 | Speract receptor repeat |
| | | | | | | proteins domain |
| | | | | | | proteins. |
| 964 | BL00420A | 20.42 | 6.400e−11 | 843-871 | BL00420 | Speract receptor repeat |
| | | | | | | proteins domain |
| | | | | | | proteins. |
| 964 | BL00420A | 20.42 | 6.607e−10 | 186-214 | BL00420 | Speract receptor repeat |
| | | | | | | proteins domain |
| | | | | | | proteins. |
| 964 | BL00420A | 20.42 | 6.754e−10 | 423-451 | BL00420 | Speract receptor repeat |
| | | | | | | proteins domain |
| | | | | | | proteins. |
| 964 | BL00420A | 20.42 | 6.815e−09 | 753-781 | BL00420 | Speract receptor repeat |
| | | | | | | proteins domain |
| | | | | | | proteins. |
| 964 | BL00420A | 20.42 | 7.049e−10 | 318-346 | BL00420 | Speract receptor repeat |
| | | | | | | proteins domain |
| | | | | | | proteins. |
| 964 | BL00420A | 20.42 | 7.092e−09 | 789-817 | BL00420 | Speract receptor repeat |
| | | | | | | proteins domain |
| | | | | | | proteins. |
| 964 | BL00420A | 20.42 | 7.231e−09 | 471-499 | BL00420 | Speract receptor repeat |
| | | | | | | proteins domain |
| | | | | | | proteins. |
| 964 | BL00420A | 20.42 | 7.344e−10 | 390-418 | BL00420 | Speract receptor repeat |
| | | | | | | proteins domain |
| | | | | | | proteins. |
| 964 | BL00420A | 20.42 | 7.923e−09 | 243-271 | BL00420 | Speract receptor repeat |
| | | | | | | proteins domain |
| | | | | | | proteins. |
| 964 | BL00420A | 20.42 | 7.923e−09 | 261-289 | BL00420 | Speract receptor repeat |
| | | | | | | proteins domain |
| | | | | | | proteins. |
| 964 | BL00420A | 20.42 | 7.923e−12 | 417-445 | BL00420 | Speract receptor repeat |
| | | | | | | proteins domain |
| | | | | | | proteins. |
| 964 | BL00420A | 20.42 | 8.082e−10 | 813-841 | BL00420 | Speract receptor repeat |
| | | | | | | proteins domain |
| | | | | | | proteins. |
| 964 | BL00420A | 20.42 | 8.200e−11 | 282-310 | BL00420 | Speract receptor repeat |
| | | | | | | proteins domain |
| | | | | | | proteins. |
| 964 | BL00420A | 20.42 | 8.230e−10 | 771-799 | BL00420 | Speract receptor repeat |
| | | | | | | proteins domain |
| | | | | | | proteins. |
| 964 | BL00420A | 20.42 | 8.338e−09 | 420-448 | BL00420 | Speract receptor repeat |
| | | | | | | proteins domain |
| | | | | | | proteins. |
| 964 | BL00420A | 20.42 | 8.615e−09 | 513-541 | BL00420 | Speract receptor repeat |
| | | | | | | proteins domain |
| | | | | | | proteins. |
| 964 | BL00420A | 20.42 | 8.754e−09 | 525-553 | BL00420 | Speract receptor repeat |
| | | | | | | proteins domain |
| 964 | BL00420A | 20.42 | 8.892e−09 | 681-709 | BL00420 | Speract receptor repeat |
| | | | | | | proteins domain |
| | | | | | | proteins. |
| 964 | BL00420A | 20.42 | 8.898e−13 | 534-562 | BL00420 | Speract receptor repeat |
| | | | | | | proteins domain |
| | | | | | | proteins. |
| 964 | BL00420A | 20.42 | 9.262e−10 | 249-277 | BL00420 | Speract receptor repeat |
| | | | | | | proteins domain |
| | | | | | | proteins. |
| 964 | BL00420A | 20.42 | 9.262e−10 | 576-604 | BL00420 | Speract receptor repeat |
| | | | | | | proteins domain |
| | | | | | | proteins. |
| 964 | BL00420A | 20.42 | 9.308e−09 | 201-229 | BL00420 | Speract receptor repeat |
| | | | | | | proteins domain |
| | | | | | | proteins. |
| 964 | BL00420A | 20.42 | 9.446e−09 | 717-745 | BL00420 | Speract receptor repeat |
| | | | | | | proteins domain |
| | | | | | | proteins. |
| 964 | BL00420A | 20.42 | 9.557e−10 | 297-325 | BL00420 | Speract receptor repeat |
| | | | | | | proteins domain |
| | | | | | | proteins. |
| 964 | BL00420A | 20.42 | 9.585e−09 | 165-193 | BL00420 | Speract receptor repeat |
| | | | | | | proteins domain |
| | | | | | | proteins. |
| 964 | BL00420A | 20.42 | 9.705e−10 | 684-712 | BL00420 | Speract receptor repeat |
| | | | | | | proteins domain |
| | | | | | | proteins. |
| 964 | BL00420A | 20.42 | 9.723e−09 | 315-343 | BL00420 | Speract receptor repeat |
| | | | | | | proteins domain |
| | | | | | | proteins. |
| 964 | BL00420A | 20.42 | 9.836e−11 | 498-526 | BL00420 | Speract receptor repeat |
| | | | | | | proteins domain |
| | | | | | | proteins. |
| 964 | BL00420A | 20.42 | 9.852e−10 | 795-823 | BL00420 | Speract receptor repeat |
| | | | | | | proteins domain |
| | | | | | | proteins. |
| 964 | BL00420A | 20.42 | 9.862e−09 | 118-146 | BL00420 | Speract receptor repeat |
| | | | | | | proteins domain |
| | | | | | | proteins. |
| 964 | BL01113A | 17.99 | 1.000e−10 | 282-308 | BL01113 | C1q domain proteins. |
| 964 | BL01113A | 17.99 | 1.000e−10 | 669-695 | BL01113 | C1q domain proteins. |
| 964 | BL01113A | 17.99 | 1.000e−11 | 591-617 | BL01113 | C1q domain proteins. |
| 964 | BL01113A | 17.99 | 1.000e−12 | 813-839 | BL01113 | C1q domain proteins. |
| 964 | BL01113A | 17.99 | 1.000e−14 | 534-560 | BL01113 | C1q domain proteins. |
| 964 | BL01113A | 17.99 | 1.173e−09 | 357-383 | BL01113 | C1q domain proteins. |
| 964 | BL01113A | 17.99 | 1.191e−10 | 462-488 | BL01113 | C1q domain proteins. |
| 964 | BL01113A | 17.99 | 1.205e−11 | 798-824 | BL01113 | C1q domain proteins. |
| 964 | BL01113A | 17.99 | 1.243e−13 | 660-686 | BL01113 | C1q domain proteins. |
| 964 | BL01113A | 17.99 | 1.519e−09 | 621-647 | BL01113 | C1q domain proteins. |
| 964 | BL01113A | 17.99 | 1.692e−12 | 789-815 | BL01113 | C1q domain proteins. |
| 964 | BL01113A | 17.99 | 1.766e−10 | 339-365 | BL01113 | C1q domain proteins. |
| 964 | BL01113A | 17.99 | 1.766e−10 | 648-674 | BL01113 | C1q domain proteins. |
| 964 | BL01113A | 17.99 | 1.957e−10 | 822-848 | BL01113 | C1q domain proteins. |
| 964 | BL01113A | 17.99 | 1.957e−10 | 879-905 | BL01113 | C1q domain proteins. |
| 964 | BL01113A | 17.99 | 2.149e−10 | 831-857 | BL01113 | C1q domain proteins. |
| 964 | BL01113A | 17.99 | 2.154e−12 | 543-569 | BL01113 | C1q domain proteins. |
| 964 | BL01113A | 17.99 | 2.212e−09 | 231-257 | BL01113 | C1q domain proteins. |
| 964 | BL01113A | 17.99 | 2.212e−09 | 327-353 | BL01113 | C1q domain proteins. |
| 964 | BL01113A | 17.99 | 2.558e−09 | 372-398 | BL01113 | C1q domain proteins. |
| 964 | BL01113A | 17.99 | 2.703e−13 | 112-138 | BL01113 | C1q domain proteins. |
| 964 | BL01113A | 17.99 | 2.723e−10 | 189-215 | BL01113 | C1q domain proteins. |
| 964 | BL01113A | 17.99 | 2.723e−10 | 219-245 | BL01113 | C1q domain proteins. |
| 964 | BL01113A | 17.99 | 2.731e−09 | 555-581 | BL01113 | C1q domain proteins. |
| 964 | BL01113A | 17.99 | 2.742e−15 | 336-362 | BL01113 | C1q domain proteins. |
| 964 | BL01113A | 17.99 | 2.846e−12 | 109-135 | BL01113 | C1q domain proteins. |
| 964 | BL01113A | 17.99 | 2.904e−09 | 207-233 | BL01113 | C1q domain proteins. |
| 964 | BL01113A | 17.99 | 2.904e−09 | 306-332 | BL01113 | C1q domain proteins. |
| 964 | BL01113A | 17.99 | 2.904e−09 | 387-413 | BL01113 | C1q domain proteins. |
| 964 | BL01113A | 17.99 | 2.904e−09 | 564-590 | BL01113 | C1q domain proteins. |
| 964 | BL01113A | 17.99 | 2.915e−10 | 210-236 | BL01113 | C1q domain proteins. |
| 964 | BL01113A | 17.99 | 2.915e−10 | 840-866 | BL01113 | C1q domain proteins. |
| 964 | BL01113A | 17.99 | 2.946e−13 | 846-872 | BL01113 | C1q domain proteins. |
| 964 | BL01113A | 17.99 | 3.077e−09 | 405-431 | BL01113 | C1q domain proteins. |
| 964 | BL01113A | 17.99 | 3.298e−10 | 756-782 | BL01113 | C1q domain proteins. |
| 964 | BL01113A | 17.99 | 3.423e−09 | 495-521 | BL01113 | C1q domain proteins. |
| 964 | BL01113A | 17.99 | 3.423e−09 | 720-746 | BL01113 | C1q domain proteins. |
| 964 | BL01113A | 17.99 | 3.455e−11 | 771-797 | BL01113 | C1q domain proteins. |
| 964 | BL01113A | 17.99 | 3.613e−15 | 115-141 | BL01113 | C1q domain proteins. |
| 964 | BL01113A | 17.99 | 4.000e−14 | 738-764 | BL01113 | C1q domain proteins. |
| 964 | BL01113A | 17.99 | 4.064e−10 | 192-218 | BL01113 | C1q domain proteins. |
| 964 | BL01113A | 17.99 | 4.064e−10 | 345-371 | BL01113 | C1q domain proteins. |
| 964 | BL01113A | 17.99 | 4.064e−10 | 723-749 | BL01113 | C1q domain proteins. |
| 964 | BL01113A | 17.99 | 4.068e−11 | 540-566 | BL01113 | C1q domain proteins. |
| 964 | BL01113A | 17.99 | 4.194e−15 | 118-144 | BL01113 | C1q domain proteins. |
| 964 | BL01113A | 17.99 | 4.231e−12 | 456-482 | BL01113 | C1q domain proteins. |
| 964 | BL01113A | 17.99 | 4.255e−10 | 324-350 | BL01113 | C1q domain proteins. |
| 964 | BL01113A | 17.99 | 4.273e−11 | 195-221 | BL01113 | C1q domain proteins. |
| 964 | BL01113A | 17.99 | 4.288e−09 | 103-129 | BL01113 | C1q domain proteins. |
| 964 | BL01113A | 17.99 | 4.288e−09 | 127-153 | BL01113 | C1q domain proteins. |
| 964 | BL01113A | 17.99 | 4.462e−09 | 246-272 | BL01113 | C1q domain proteins. |
| 964 | BL01113A | 17.99 | 4.635e−09 | 204-230 | BL01113 | C1q domain proteins. |
| 964 | BL01113A | 17.99 | 4.638e−10 | 480-506 | BL01113 | C1q domain proteins. |
| 964 | BL01113A | 17.99 | 4.774e−15 | 297-323 | BL01113 | C1q domain proteins. |
| 964 | BL01113A | 17.99 | 4.808e−09 | 381-407 | BL01113 | C1q domain proteins. |
| 964 | BL01113A | 17.99 | 4.886e−11 | 249-275 | BL01113 | C1q domain proteins. |
| 964 | BL01113A | 17.99 | 4.923e−12 | 531-557 | BL01113 | C1q domain proteins. |
| 964 | BL01113A | 17.99 | 4.981e−09 | 375-401 | BL01113 | C1q domain proteins. |
| 964 | BL01113A | 17.99 | 4.981e−09 | 837-863 | BL01113 | C1q domain proteins. |
| 964 | BL01113A | 17.99 | 5.021e−10 | 705-731 | BL01113 | C1q domain proteins. |
| 964 | BL01113A | 17.99 | 5.091e−11 | 645-671 | BL01113 | C1q domain proteins. |
| 964 | BL01113A | 17.99 | 5.091e−11 | 762-788 | BL01113 | C1q domain proteins. |
| 964 | BL01113A | 17.99 | 5.091e−14 | 807-833 | BL01113 | C1q domain proteins. |
| 964 | BL01113A | 17.99 | 5.385e−12 | 525-551 | BL01113 | C1q domain proteins. |
| 964 | BL01113A | 17.99 | 5.404e−10 | 291-317 | BL01113 | C1q domain proteins. |
| 964 | BL01113A | 17.99 | 5.404e−10 | 852-878 | BL01113 | C1q domain proteins. |
| 964 | BL01113A | 17.99 | 5.500e−11 | 573-599 | BL01113 | C1q domain proteins. |
| 964 | BL01113A | 17.99 | 5.615e−12 | 693-719 | BL01113 | C1q domain proteins. |
| 964 | BL01113A | 17.99 | 5.673e−09 | 447-473 | BL01113 | C1q domain proteins. |
| 964 | BL01113A | 17.99 | 5.673e−09 | 489-515 | BL01113 | C1q domain proteins. |
| 964 | BL01113A | 17.99 | 5.846e−12 | 546-572 | BL01113 | C1q domain proteins. |
| 964 | BL01113A | 17.99 | 5.909e−11 | 582-608 | BL01113 | C1q domain proteins. |
| 964 | BL01113A | 17.99 | 5.979e−10 | 471-497 | BL01113 | C1q domain proteins. |
| 964 | BL01113A | 17.99 | 6.108e−13 | 237-263 | BL01113 | C1q domain proteins. |
| 964 | BL01113A | 17.99 | 6.114e−11 | 804-830 | BL01113 | C1q domain proteins. |
| 964 | BL01113A | 17.99 | 6.318e−11 | 216-242 | BL01113 | C1q domain proteins. |
| 964 | BL01113A | 17.99 | 6.362e−10 | 513-539 | BL01113 | C1q domain proteins. |
| 964 | BL01113A | 17.99 | 6.365e−09 | 333-359 | BL01113 | C1q domain proteins. |
| 964 | BL01113A | 17.99 | 6.365e−09 | 816-842 | BL01113 | C1q domain proteins. |
| 964 | BL01113A | 17.99 | 6.523e−11 | 708-734 | BL01113 | C1q domain proteins. |
| 964 | BL01113A | 17.99 | 6.538e−09 | 732-758 | BL01113 | C1q domain proteins. |
| 964 | BL01113A | 17.99 | 6.538e−12 | 171-197 | BL01113 | C1q domain proteins. |
| 964 | BL01113A | 17.99 | 6.712e−09 | 408-434 | BL01113 | C1q domain proteins. |
| 964 | BL01113A | 17.99 | 6.712e−09 | 603-629 | BL01113 | C1q domain proteins. |
| 964 | BL01113A | 17.99 | 6.745e−10 | 234-260 | BL01113 | C1q domain proteins. |
| 964 | BL01113A | 17.99 | 6.769e−12 | 636-662 | BL01113 | C1q domain proteins. |
| 964 | BL01113A | 17.99 | 6.936e−10 | 777-803 | BL01113 | C1q domain proteins. |
| 964 | BL01113A | 17.99 | 7.128e−10 | 702-728 | BL01113 | C1q domain proteins. |
| 964 | BL01113A | 17.99 | 7.128e−10 | 867-893 | BL01113 | C1q domain proteins. |
| 964 | BL01113A | 17.99 | 7.136e−11 | 537-563 | BL01113 | C1q domain proteins. |
| 964 | BL01113A | 17.99 | 7.231e−09 | 561-587 | BL01113 | C1q domain proteins. |
| 964 | BL01113A | 17.99 | 7.231e−09 | 681-707 | BL01113 | C1q domain proteins. |
| 964 | BL01113A | 17.99 | 7.231e−09 | 855-881 | BL01113 | C1q domain proteins. |
| 964 | BL01113A | 17.99 | 7.273e−14 | 714-740 | BL01113 | C1q domain proteins. |
| 964 | BL01113A | 17.99 | 7.319e−10 | 876-902 | BL01113 | C1q domain proteins. |
| 964 | BL01113A | 17.99 | 7.324e−13 | 186-212 | BL01113 | C1q domain proteins. |
| 964 | BL01113A | 17.99 | 7.404e−09 | 528-554 | BL01113 | C1q domain proteins. |
| 964 | BL01113A | 17.99 | 7.511e−10 | 121-147 | BL01113 | C1q domain proteins. |
| 964 | BL01113A | 17.99 | 7.511e−10 | 657-683 | BL01113 | C1q domain proteins. |
| 964 | BL01113A | 17.99 | 7.545e−11 | 330-356 | BL01113 | C1q domain proteins. |
| 964 | BL01113A | 17.99 | 7.577e−09 | 711-737 | BL01113 | C1q domain proteins. |
| 964 | BL01113A | 17.99 | 7.692e−12 | 729-755 | BL01113 | C1q domain proteins. |
| 964 | BL01113A | 17.99 | 7.692e−12 | 747-773 | BL01113 | C1q domain proteins. |
| 964 | BL01113A | 17.99 | 7.702e−10 | 768-794 | BL01113 | C1q domain proteins. |
| 964 | BL01113A | 17.99 | 7.750e−09 | 684-710 | BL01113 | C1q domain proteins. |
| 964 | BL01113A | 17.99 | 7.750e−09 | 792-818 | BL01113 | C1q domain proteins. |
| 964 | BL01113A | 17.99 | 7.923e−09 | 402-428 | BL01113 | C1q domain proteins. |
| 964 | BL01113A | 17.99 | 7.923e−09 | 549-575 | BL01113 | C1q domain proteins. |
| 964 | BL01113A | 17.99 | 7.923e−09 | 627-653 | BL01113 | C1q domain proteins. |
| 964 | BL01113A | 17.99 | 7.923e−09 | 663-689 | BL01113 | C1q domain proteins. |
| 964 | BL01113A | 17.99 | 7.923e−12 | 180-206 | BL01113 | C1q domain proteins. |
| 964 | BL01113A | 17.99 | 8.054e−13 | 567-593 | BL01113 | C1q domain proteins. |
| 964 | BL01113A | 17.99 | 8.085e−10 | 861-887 | BL01113 | C1q domain proteins. |
| 964 | BL01113A | 17.99 | 8.096e−09 | 315-341 | BL01113 | C1q domain proteins. |
| 964 | BL01113A | 17.99 | 8.096e−09 | 717-743 | BL01113 | C1q domain proteins. |
| 964 | BL01113A | 17.99 | 8.159e−11 | 600-626 | BL01113 | C1q domain proteins. |
| 964 | BL01113A | 17.99 | 8.277e−10 | 642-668 | BL01113 | C1q domain proteins. |
| 964 | BL01113A | 17.99 | 8.297e−13 | 240-266 | BL01113 | C1q domain proteins. |
| 964 | BL01113A | 17.99 | 8.385e−12 | 124-150 | BL01113 | C1q domain proteins. |
| 964 | BL01113A | 17.99 | 8.442e−09 | 774-800 | BL01113 | C1q domain proteins. |
| 964 | BL01113A | 17.99 | 8.468e−10 | 873-899 | BL01113 | C1q domain proteins. |
| 964 | BL01113A | 17.99 | 8.548e−15 | 174-200 | BL01113 | C1q domain proteins. |
| 964 | BL01113A | 17.99 | 8.548e−15 | 672-698 | BL01113 | C1q domain proteins. |
| 964 | BL01113A | 17.99 | 8.636e−14 | 384-410 | BL01113 | C1q domain proteins. |
| 964 | BL01113A | 17.99 | 8.773e−11 | 522-548 | BL01113 | C1q domain proteins. |
| 964 | BL01113A | 17.99 | 8.773e−11 | 552-578 | BL01113 | C1q domain proteins. |
| 964 | BL01113A | 17.99 | 8.788e−09 | 252-278 | BL01113 | C1q domain proteins. |
| 964 | BL01113A | 17.99 | 8.846e−12 | 177-203 | BL01113 | C1q domain proteins. |
| 964 | BL01113A | 17.99 | 9.043e−10 | 366-392 | BL01113 | C1q domain proteins. |
| 964 | BL01113A | 17.99 | 9.129e−15 | 183-209 | BL01113 | C1q domain proteins. |
| 964 | BL01113A | 17.99 | 9.135e−09 | 243-269 | BL01113 | C1q domain proteins. |
| 964 | BL01113A | 17.99 | 9.135e−09 | 465-491 | BL01113 | C1q domain proteins. |
| 964 | BL01113A | 17.99 | 9.182e−14 | 651-677 | BL01113 | C1q domain proteins. |
| 964 | BL01113A | 17.99 | 9.234e−10 | 501-527 | BL01113 | C1q domain proteins. |
| 964 | BL01113A | 17.99 | 9.308e−12 | 198-224 | BL01113 | C1q domain proteins. |
| 964 | BL01113A | 17.99 | 9.308e−12 | 516-542 | BL01113 | C1q domain proteins. |
| 964 | BL01113A | 17.99 | 9.386e−11 | 228-254 | BL01113 | C1q domain proteins. |
| 964 | BL01113A | 17.99 | 9.455e−14 | 426-452 | BL01113 | C1q domain proteins. |
| 964 | BL01113A | 17.99 | 9.538e−12 | 106-132 | BL01113 | C1q domain proteins. |
| 964 | BL01113A | 17.99 | 9.617e−10 | 606-632 | BL01113 | C1q domain proteins. |
| 964 | BL01113A | 17.99 | 9.654e−09 | 585-611 | BL01113 | C1q domain proteins. |
| 964 | BL01113A | 17.99 | 9.654e−09 | 843-869 | BL01113 | C1q domain proteins. |
| 964 | BL01113A | 17.99 | 9.757e−13 | 417-443 | BL01113 | C1q domain proteins. |
| 964 | BL01113A | 17.99 | 9.757e−13 | 435-461 | BL01113 | C1q domain proteins. |
| 964 | BL01113A | 17.99 | 9.809e−10 | 558-584 | BL01113 | C1q domain proteins. |
| 964 | BL01113A | 17.99 | 9.809e−10 | 699-725 | BL01113 | C1q domain proteins. |
| 964 | BL01208B | 15.83 | 1.900e−13 | 77-91 | BL01208 | VWFC domain proteins. |
| 964 | DM01418A | 20.83 | 1.000e−40 | 955-1002 | DM01418 | 352 FIBRILLAR COLLAGEN |
| | | | | | | CARBOXYL-TERMINAL. |
| 964 | DM01418B | 22.51 | 1.000e−40 | 1053-1094 | DM01418 | 352 FIBRILLAR COLLAGEN |
| | | | | | | CARBOXYL-TERMINAL. |
| 964 | DM01418C | 20.48 | 1.000e−40 | 1124-1165 | DM01418 | 352 FIBRILLAR COLLAGEN |
| | | | | | | CARBOXYL-TERMINAL. |
| 964 | PR00049D | 0.00 | 7.559e−09 | 176-190 | PR00049 | WILM'S TUMOUR PROTEIN |
| | | | | | | SIGNATURE |
| 964 | PR00524F | 5.36 | 8.468e−09 | 513-526 | PR00524 | CHOLECYSTOKININ TYPE A |
| | | | | | | RECEPTOR SIGNATURE |
| 965 | BL00039A | 18.44 | 4.600e−31 | 211-249 | BL00039 | DEAD-box subfamily ATP- |
| | | | | | | dependent helicases |
| | | | | | | proteins. |
| 965 | BL00039B | 19.19 | 4.913e−17 | 269-294 | BL00039 | DEAD-box subfamily ATP- |
| | | | | | | dependent helicases |
| | | | | | | proteins. |
| 965 | BL00039C | 15.63 | 8.000e−27 | 353-376 | BL00039 | DEAD-box subfamily ATP- |
| | | | | | | dependent helicases |
| | | | | | | proteins. |
| 965 | BL00039D | 21.67 | 1.783e−40 | 506-551 | BL00039 | DEAD-box subfamily ATP- |
| | | | | | | dependent helicases |
| | | | | | | proteins. |
| 965 | BL00048 | 6.39 | 6.400e−09 | 612-638 | BL00048 | Protamine P1 proteins. |
| 965 | BL00291A | 4.49 | 9.458e−10 | 630-664 | BL00291 | Prion protein. |
| 965 | PF00271 | 7.99 | 3.250e−09 | 537-544 | PF00271 | Helicases conserved C- |
| | | | | | | terminal domain |
| | | | | | | proteins. |
| 966 | PR00308C | 3.83 | 2.169e−09 | 10-19 | PR00308 | TYPE I ANTIFREEZE |
| | | | | | | PROTEIN SIGNATURE |
| 966 | PR00456E | 3.06 | 6.625e−10 | 12-26 | PR00456 | RIBOSOMAL PROTEIN P2 |
| | | | | | | SIGNATURE |
| 970 | BL00460A | 28.67 | 1.514e−24 | 57-91 | BL00460 | Glutathione peroxidases |
| | | | | | | selenocysteine |
| | | | | | | proteins. |
| 970 | BL00460B | 9.73 | 4.600e−23 | 99-116 | BL00460 | Glutathione peroxidases |
| | | | | | | selenocysteine |
| | | | | | | proteins. |
| 970 | BL00460C | 14.35 | 4.724e−21 | 137-159 | BL00460 | Glutathione peroxidases |
| | | | | | | selenocysteine |
| | | | | | | proteins. |
| 970 | BL00460D | 16.89 | 1.818e−22 | 185-204 | BL00460 | Glutathione peroxidases |
| | | | | | | selenocysteine |
| | | | | | | proteins. |
| 971 | BL00262A | 12.48 | 4.600e−15 | 124-141 | BL00262 | Insulin family |
| | | | | | | proteins. |
| 971 | BL00262B | 16.89 | 8.286e−17 | 160-179 | BL00262 | Insulin family |
| | | | | | | proteins. |
| 971 | PR00276A | 11.84 | 4.750e−13 | 161-170 | PR00276 | INSULIN A CHAIN |
| | | | | | | SIGNATURE |
| 971 | PR00276B | 8.02 | 7.828e−10 | 170-179 | PR00276 | INSULIN A CHAIN |
| | | | | | | SIGNATURE |
| 971 | PR00277A | 14.82 | 2.421e−13 | 121-134 | PR00277 | INSULIN B CHAIN |
| | | | | | | SIGNATURE |
| 971 | PR00277B | 12.79 | 2.350e−11 | 135-147 | PR00277 | INSULIN B CHAIN |
| | | | | | | SIGNATURE |
| 975 | PF00023A | 16.03 | 5.500e−10 | 132-147 | PF00023 | Ank repeat proteins. |
| 975 | PF00791B | 28.49 | 1.818e−11 | 132-186 | PF00791 | Domain present in ZO-1 |
| | | | | | | and Unc5-like netrin |
| | | | | | | receptors. |
| 975 | PF00791B | 28.49 | 5.046e−09 | 165-219 | PF00791 | Domain present in ZO-1 |
| | | | | | | and Unc5-like netrin |
| | | | | | | receptors. |
| 975 | PF00791B | 28.49 | 6.942e−10 | 99-153 | PF00791 | Domain present in ZO-1 |
| | | | | | | and Unc5-like netrin |
| | | | | | | receptors. |
| 979 | PF00777A | 8.25 | 4.000e−10 | 52-63 | PF00777 | Sialyltransferase |
| | | | | | | family. |
| 979 | PF00777B | 29.69 | 1.500e−28 | 89-131 | PF00777 | Sialyltransferase |
| | | | | | | family. |
| 979 | PF00777C | 18.60 | 1.000e−40 | 158-212 | PF00777 | Sialyltransferase |
| | | | | | | family. |
| 979 | PF00777D | 22.05 | 9.325e−39 | 291-336 | PF00777 | Sialyltransferase |
| | | | | | | family. |
| 980 | BL00171B | 26.59 | 1.125e−16 | 69-119 | BL00171 | Triosephosphate |
| | | | | | | isomerase proteins. |
| 980 | BL00171C | 14.53 | 6.294e−36 | 163-195 | BL00171 | Triosephosphate |
| | | | | | | isomerase proteins. |
| 980 | BL00171D | 15.75 | 1.563e−37 | 211-245 | BL00171 | Triosephosphate |
| | | | | | | isomerase proteins. |
| 981 | BL00061A | 9.41 | 4.600e−13 | 139-149 | BL00061 | Short-chain |
| | | | | | | dehydrogenases/reductases |
| | | | | | | family proteins. |
| 981 | BL00061B | 25.79 | 9.250e−27 | 187-224 | BL00061 | Short-chain |
| | | | | | | dehydrogenases/reductases |
| | | | | | | family proteins. |
| 981 | BL00061C | 7.86 | 1.563e−10 | 231-240 | BL00061 | Short-chain |
| | | | | | | dehydrogenases/reductases |
| | | | | | | family proteins. |
| 981 | PR00080A | 9.32 | 3.143e−12 | 139-150 | PR00080 | ALCOHOL DEHYDROGENASE |
| | | | | | | SUPERFAMILY SIGNATURE |
| 981 | PR00080C | 17.16 | 4.429e−13 | 207-226 | PR00080 | ALCOHOL DEHYDROGENASE |
| | | | | | | SUPERFAMILY SIGNATURE |
| 981 | PR00081A | 10.53 | 4.333e−14 | 63-80 | PR00081 | GLUCOSE/RIBITOL |
| | | | | | | DEHYDROGENASE FAMILY |
| | | | | | | SIGNATURE |
| 981 | PR00081B | 10.38 | 8.800e−14 | 139-150 | PR00081 | GLUCOSE/RIBITOL |
| | | | | | | DEHYDROGENASE FAMILY |
| | | | | | | SIGNATURE |
| 981 | PR00081D | 15.80 | 5.800e−12 | 207-226 | PR00081 | GLUCOSE/RIBITOL |
| | | | | | | DEHYDROGENASE FAMILY |
| | | | | | | SIGNATURE |
| 981 | PR00081E | 17.54 | 9.250e−12 | 230-247 | PR00081 | GLUCOSE/RIBITOL |
| | | | | | | DEHYDROGENASE FAMILY |
| | | | | | | SIGNATURE |
| 982 | BL01073 | 24.30 | 1.000e−40 | 51-100 | BL01073 | Ribosomal protein L24e |
| | | | | | | proteins. |
| 985 | BL00223A | 15.59 | 2.253e−11 | 298-331 | BL00223 | Annexins repeat |
| | | | | | | proteins domain |
| | | | | | | proteins. |
| 985 | BL00223A | 15.59 | 8.250e−27 | 138-171 | BL00223 | Annexins repeat |
| | | | | | | proteins domain |
| | | | | | | proteins. |
| 985 | BL00223A | 15.59 | 8.750e−27 | 66-99 | BL00223 | Annexins repeat |
| | | | | | | proteins domain |
| | | | | | | proteins. |
| 985 | BL00223B | 28.47 | 3.032e−37 | 208-257 | BL00223 | Annexins repeat |
| | | | | | | proteins domain |
| | | | | | | proteins. |
| 985 | BL00223C | 24.79 | 1.000e−40 | 285-339 | BL00223 | Annexins repeat |
| | | | | | | proteins domain |
| | | | | | | proteins. |
| 985 | BL00223C | 24.79 | 2.735e−15 | 125-179 | BL00223 | Annexins repeat |
| | | | | | | proteins domain |
| | | | | | | proteins. |
| 985 | BL00223C | 24.79 | 9.438e−16 | 53-107 | BL00223 | Annexins repeat |
| | | | | | | proteins domain |
| | | | | | | proteins. |
| 985 | PR00196A | 11.16 | 4.676e−10 | 147-169 | PR00196 | ANNEXIN FAMILY |
| | | | | | | SIGNATURE |
| 985 | PR00196A | 11.16 | 8.773e−23 | 75-97 | PR00196 | ANNEXIN FAMILY |
| | | | | | | SIGNATURE |
| 985 | PR00196B | 10.68 | 5.050e−17 | 115-131 | PR00196 | ANNEXIN FAMILY |
| | | | | | | SIGNATURE |
| 985 | PR00196C | 10.36 | 1.443e−13 | 70-91 | PR00196 | ANNEXIN FAMILY |
| | | | | | | SIGNATURE |
| 985 | PR00196C | 10.36 | 7.968e−21 | 142-163 | PR00196 | ANNEXIN FAMILY |
| | | | | | | SIGNATURE |
| 985 | PR00196C | 10.36 | 9.609e−11 | 302-323 | PR00196 | ANNEXIN FAMILY |
| | | | | | | SIGNATURE |
| 985 | PR00196D | 21.86 | 1.818e−23 | 226-252 | PR00196 | ANNEXIN FAMILY |
| | | | | | | SIGNATURE |
| 985 | PR00196E | 9.19 | 1.900e−09 | 74-94 | PR00196 | ANNEXIN FAMILY |
| | | | | | | SIGNATURE |
| 985 | PR00196E | 9.19 | 9.100e−22 | 306-326 | PR00196 | ANNEXIN FAMILY |
| | | | | | | SIGNATURE |
| 985 | PR00196E | 9.19 | 9.763e−10 | 146-166 | PR00196 | ANNEXIN FAMILY |
| | | | | | | SIGNATURE |
| 985 | PR00196F | 13.89 | 1.720e−14 | 334-349 | PR00196 | ANNEXIN FAMILY |
| | | | | | | SIGNATURE |
| 985 | PR00196G | 11.72 | 9.333e−14 | 350-363 | PR00196 | ANNEXIN FAMILY |
| | | | | | | SIGNATURE |
| 985 | PR00197B | 7.56 | 9.786e−12 | 75-97 | PR00197 | ANNEXIN TYPE I |
| | | | | | | SIGNATURE |
| 985 | PR00197D | 7.50 | 6.242e−10 | 302-323 | PR00197 | ANNEXIN TYPE I |
| | | | | | | SIGNATURE |
| 985 | PR00197D | 7.50 | 7.207e−11 | 70-91 | PR00197 | ANNEXIN TYPE I |
| | | | | | | SIGNATURE |
| 985 | PR00197D | 7.50 | 8.500e−15 | 142-163 | PR00197 | ANNEXIN TYPE I |
| | | | | | | SIGNATURE |
| 985 | PR00197E | 11.89 | 3.415e−14 | 226-252 | PR00197 | ANNEXIN TYPE I |
| | | | | | | SIGNATURE |
| 985 | PR00197F | 9.03 | 5.757e−17 | 306-326 | PR00197 | ANNEXIN TYPE I |
| | | | | | | SIGNATURE |
| 985 | PR00197F | 9.03 | 9.724e−09 | 146-166 | PR00197 | ANNEXIN TYPE I |
| | | | | | | SIGNATURE |
| 985 | PR00198B | 8.71 | 5.400e−12 | 75-97 | PR00198 | ANNEXIN TYPE II |
| | | | | | | SIGNATURE |
| 985 | PR00198D | 7.65 | 2.113e−10 | 302-323 | PR00198 | ANNEXIN TYPE II |
| | | | | | | SIGNATURE |
| 985 | PR00198D | 7.65 | 4.812e−13 | 142-163 | PR00198 | ANNEXIN TYPE II |
| | | | | | | SIGNATURE |
| 985 | PR00198D | 7.65 | 6.155e−09 | 70-91 | PR00198 | ANNEXIN TYPE II |
| | | | | | | SIGNATURE |
| 985 | PR00198E | 14.67 | 7.698e−14 | 226-252 | PR00198 | ANNEXIN TYPE II |
| | | | | | | SIGNATURE |
| 985 | PR00198G | 8.09 | 9.407e−10 | 146-166 | PR00198 | ANNEXIN TYPE II |
| | | | | | | SIGNATURE |
| 985 | PR00198G | 8.09 | 9.486e−16 | 306-326 | PR00198 | ANNEXIN TYPE II |
| | | | | | | SIGNATURE |
| 985 | PR00198H | 12.05 | 3.025e−09 | 350-363 | PR00198 | ANNEXIN TYPE II |
| | | | | | | SIGNATURE |
| 985 | PR00199B | 6.86 | 9.316e−17 | 75-97 | PR00199 | ANNEXIN TYPE III |
| | | | | | | SIGNATURE |
| 985 | PR00199B | 6.86 | 9.518e−09 | 147-169 | PR00199 | ANNEXIN TYPE III |
| | | | | | | SIGNATURE |
| 985 | PR00199C | 13.84 | 5.680e−11 | 115-131 | PR00199 | ANNEXIN TYPE III |
| | | | | | | SIGNATURE |
| 985 | PR00199D | 5.65 | 6.013e−12 | 142-163 | PR00199 | ANNEXIN TYPE III |
| | | | | | | SIGNATURE |
| 985 | PR00199D | 5.65 | 9.609e−09 | 302-323 | PR00199 | ANNEXIN TYPE III |
| | | | | | | SIGNATURE |
| 985 | PR00199D | 5.65 | 9.750e−14 | 70-91 | PR00199 | ANNEXIN TYPE III |
| | | | | | | SIGNATURE |
| 985 | PR00199F | 16.19 | 5.565e−15 | 226-252 | PR00199 | ANNEXIN TYPE III |
| | | | | | | SIGNATURE |
| 985 | PR00199G | 9.09 | 5.821e−14 | 307-332 | PR00199 | ANNEXIN TYPE III |
| | | | | | | SIGNATURE |
| 985 | PR00199H | 12.62 | 5.632e−09 | 350-363 | PR00199 | ANNEXIN TYPE III |
| | | | | | | SIGNATURE |
| 985 | PR00200B | 7.39 | 1.429e−10 | 147-169 | PR00200 | ANNEXIN TYPE IV |
| | | | | | | SIGNATURE |
| 985 | PR00200B | 7.39 | 9.372e−15 | 75-97 | PR00200 | ANNEXIN TYPE IV |
| | | | | | | SIGNATURE |
| 985 | PR00200D | 10.01 | 8.012e−13 | 115-131 | PR00200 | ANNEXIN TYPE IV |
| | | | | | | SIGNATURE |
| 985 | PR00200E | 10.00 | 4.522e−11 | 70-91 | PR00200 | ANNEXIN TYPE IV |
| | | | | | | SIGNATURE |
| 985 | PR00200E | 10.00 | 6.157e−12 | 302-323 | PR00200 | ANNEXIN TYPE IV |
| | | | | | | SIGNATURE |
| 985 | PR00200E | 10.00 | 7.446e−17 | 142-163 | PR00200 | ANNEXIN TYPE IV |
| | | | | | | SIGNATURE |
| 985 | PR00200F | 13.72 | 1.228e−17 | 226-252 | PR00200 | ANNEXIN TYPE IV |
| | | | | | | SIGNATURE |
| 985 | PR00200G | 9.43 | 3.473e−16 | 306-332 | PR00200 | ANNEXIN TYPE IV |
| | | | | | | SIGNATURE |
| 985 | PR00200G | 9.43 | 6.218e−09 | 146-172 | PR00200 | ANNEXIN TYPE IV |
| | | | | | | SIGNATURE |
| 985 | PR00200H | 13.68 | 9.775e−10 | 350-363 | PR00200 | ANNEXIN TYPE IV |
| | | | | | | SIGNATURE |
| 985 | PR00201A | 6.05 | 4.623e−15 | 75-97 | PR00201 | ANNEXIN TYPE V |
| | | | | | | SIGNATURE |
| 985 | PR00201A | 6.05 | 8.958e−10 | 147-169 | PR00201 | ANNEXIN TYPE V |
| | | | | | | SIGNATURE |
| 985 | PR00201C | 11.13 | 1.947e−17 | 115-131 | PR00201 | ANNEXIN TYPE V |
| | | | | | | SIGNATURE |
| 985 | PR00201D | 10.49 | 5.330e−09 | 302-323 | PR00201 | ANNEXIN TYPE V |
| | | | | | | SIGNATURE |
| 985 | PR00201D | 10.49 | 6.764e−13 | 142-163 | PR00201 | ANNEXIN TYPE V |
| | | | | | | SIGNATURE |
| 985 | PR00201E | 12.37 | 9.780e−17 | 226-252 | PR00201 | ANNEXIN TYPE V |
| | | | | | | SIGNATURE |
| 985 | PR00201G | 11.02 | 5.375e−21 | 306-332 | PR00201 | ANNEXIN TYPE V |
| | | | | | | SIGNATURE |
| 985 | PR00201H | 12.04 | 9.859e−12 | 350-363 | PR00201 | ANNEXIN TYPE V |
| | | | | | | SIGNATURE |
| 985 | PR00202B | 11.44 | 8.590e−15 | 74-97 | PR00202 | ANNEXIN TYPE VI |
| | | | | | | SIGNATURE |
| 985 | PR00202C | 13.34 | 5.438e−16 | 115-131 | PR00202 | ANNEXIN TYPE VI |
| | | | | | | SIGNATURE |
| 985 | PR00202D | 5.58 | 8.813e−13 | 142-163 | PR00202 | ANNEXIN TYPE VI |
| | | | | | | SIGNATURE |
| 985 | PR00202E | 13.00 | 6.663e−14 | 226-252 | PR00202 | ANNEXIN TYPE VI |
| | | | | | | SIGNATURE |
| 985 | PR00202G | 8.01 | 5.703e−10 | 146-172 | PR00202 | ANNEXIN TYPE VI |
| | | | | | | SIGNATURE |
| 985 | PR00202G | 8.01 | 6.357e−18 | 306-332 | PR00202 | ANNEXIN TYPE VI |
| | | | | | | SIGNATURE |
| 985 | PR00202H | 9.20 | 3.588e−10 | 350-363 | PR00202 | ANNEXIN TYPE VI |
| | | | | | | SIGNATURE |
| 990 | BL01159 | 13.85 | 5.776e−10 | 166-180 | BL01159 | WW/rsp5/WWP domain |
| | | | | | | proteins. |
| 990 | PR00403A | 16.82 | 9.561e−09 | 151-164 | PR00403 | WW DOMAIN SIGNATURE |
| 990 | PR00403B | 12.19 | 1.184e−11 | 166-180 | PR00403 | WW DOMAIN SIGNATURE |
| 991 | BL01182F | 10.07 | 4.162e−16 | 9-23 | BL01182 | Glycosyl hydrolases |
| | | | | | | family 35 proteins. |
| 991 | PR00742I | 13.54 | 6.455e−18 | 9-25 | PR00742 | GLYCOSYL HYDROLASE |
| | | | | | | FAMILY 35 SIGNATURE |
| 994 | BL00563A | 16.73 | 7.097e−40 | 26-64 | BL00563 | Stathmin family |
| | | | | | | proteins. |
| 994 | BL00563B | 6.08 | 1.000e−32 | 65-91 | BL00563 | Stathmin family |
| | | | | | | proteins. |
| 994 | BL00563C | 12.83 | 2.174e−37 | 95-124 | BL00563 | Stathmin family |
| | | | | | | proteins. |
| 994 | BL00563D | 11.38 | 1.000e−40 | 125-160 | BL00563 | Stathmin family |
| | | | | | | proteins. |
| 994 | BL00563D | 11.38 | 9.609e−09 | 74-109 | BL00563 | Stathmin family |
| | | | | | | proteins. |
| 994 | PR00345A | 13.46 | 4.316e−22 | 30-48 | PR00345 | STATHMIN FAMILY |
| | | | | | | SIGNATURE |
| 994 | PR00345B | 7.12 | 7.750e−35 | 59-87 | PR00345 | STATHMIN FAMILY |
| | | | | | | SIGNATURE |
| 994 | PR00345C | 4.54 | 5.714e−29 | 88-111 | PR00345 | STATHMIN FAMILY |
| | | | | | | SIGNATURE |
| 994 | PR00345D | 10.97 | 6.850e−31 | 112-135 | PR00345 | STATHMIN FAMILY |
| | | | | | | SIGNATURE |
| 994 | PR00345E | 8.54 | 3.571e−29 | 136-160 | PR00345 | STATHMIN FAMILY |
| | | | | | | SIGNATURE |
| 998 | PF00846A | 11.74 | 5.537e−09 | 108-154 | PF00846 | Hantavirus nucleocapsid |
| | | | | | | protein. |
| 999 | BL00142 | 8.38 | 9.438e−10 | 528-538 | BL00142 | Neutral zinc |
| | | | | | | metallopeptidases, |
| | | | | | | zinc-binding region |
| | | | | | | proteins. |
| 999 | PR00756A | 12.90 | 6.500e−18 | 367-382 | PR00756 | MEMBRANE ALANYL |
| | | | | | | DIPEPTIDASE (M1) FAMILY |
| | | | | | | SIGNATURE |
| 999 | PR00756B | 14.06 | 4.000e−14 | 415-430 | PR00756 | MEMBRANE ALANYL |
| | | | | | | DIPEPTIDASE (M1) FAMILY |
| | | | | | | SIGNATURE |
| 999 | PR00756C | 11.60 | 6.087e−13 | 492-502 | PR00756 | MEMBRANE ALANYL |
| | | | | | | DIPEPTIDASE (M1) FAMILY |
| | | | | | | SIGNATURE |
| 999 | PR00756D | 10.58 | 8.412e−21 | 528-543 | PR00756 | MEMBRANE ALANYL |
| | | | | | | DIPEPTIDASE (M1) FAMILY |
| | | | | | | SIGNATURE |
| 999 | PR00756E | 11.91 | 4.375e−16 | 547-559 | PR00756 | MEMBRANE ALANYL |
| | | | | | | DIPEPTIDASE (M1) FAMILY |
| | | | | | | SIGNATURE |
| 1000 | BL00139A | 10.29 | 8.457e−11 | 277-286 | BL00139 | Eukaryotic thiol |
| | | | | | | (cysteine) proteases |
| | | | | | | cysteine proteins. |
| 1000 | BL00139C | 9.23 | 3.769e−12 | 429-438 | BL00139 | Eukaryotic thiol |
| | | | | | | (cysteine) proteases |
| | | | | | | cysteine proteins. |
| 1000 | BL00139D | 9.24 | 3.769e−21 | 447-463 | BL00139 | Eukaryotic thiol |
| | | | | | | (cysteine) proteases |
| | | | | | | cysteine proteins. |
| 1000 | PR00705A | 10.55 | 6.000e−13 | 277-292 | PR00705 | PAPAIN CYSTEINE |
| | | | | | | PROTEASE (C1) FAMILY |
| | | | | | | SIGNATURE |
| 1000 | PR00705B | 10.22 | 6.143e−12 | 430-440 | PR00705 | PAPAIN CYSTEINE |
| | | | | | | PROTEASE (C1) FAMILY |
| | | | | | | SIGNATURE |
| 1000 | PR00705C | 11.87 | 6.824e−09 | 447-453 | PR00705 | PAPAIN CYSTEINE |
| | | | | | | PROTEASE (C1) FAMILY |
| | | | | | | SIGNATURE |
| 1001 | PF01008A | 20.14 | 9.333e−14 | 144-164 | PF01008 | Initiation factor 2 |
| | | | | | | subunit. |
| 1001 | PF01008B | 25.59 | 1.964e−32 | 192-234 | PF01008 | Initiation factor 2 |
| | | | | | | subunit. |
| 1001 | PF01008C | 12.25 | 9.667e−18 | 273-292 | PF01008 | Initiation factor 2 |
| | | | | | | subunit. |
| 1002 | BL01164A | 19.74 | 6.063e−23 | 324-353 | BL01164 | Copper amine oxidase |
| | | | | | | topaquinone proteins. |
| 1002 | BL01164B | 19.21 | 1.818e−21 | 364-390 | BL01164 | Copper amine oxidase |
| | | | | | | topaquinone proteins. |
| 1002 | BL01164C | 17.88 | 1.563e−23 | 391-419 | BL01164 | Copper amine oxidase |
| | | | | | | topaquinone proteins. |
| 1002 | BL01164D | 19.79 | 1.333e−33 | 470-504 | BL01164 | Copper amine oxidase |
| | | | | | | topaquinone proteins. |
| 1002 | BL01164E | 13.77 | 6.000e−18 | 530-547 | BL01164 | Copper amine oxidase |
| | | | | | | topaquinone proteins. |
| 1002 | BL01164F | 19.03 | 5.091e−28 | 677-706 | BL01164 | Copper amine oxidase |
| | | | | | | topaquinone proteins. |
| 1002 | PR00766A | 16.19 | 5.500e−23 | 86-105 | PR00766 | AMILORIDE-SENSITIVE |
| | | | | | | AMINE OXIDASE SIGNATURE |
| 1002 | PR00766B | 10.58 | 4.706e−25 | 115-134 | PR00766 | AMILORIDE-SENSITIVE |
| | | | | | | AMINE OXIDASE SIGNATURE |
| 1002 | PR00766C | 15.50 | 4.130e−23 | 204-224 | PR00766 | AMILORIDE-SENSITIVE |
| | | | | | | AMINE OXIDASE SIGNATURE |
| 1002 | PR00766D | 9.15 | 1.000e−26 | 227-247 | PR00766 | AMILORIDE-SENSITIVE |
| | | | | | | AMINE OXIDASE SIGNATURE |
| 1002 | PR00766E | 9.59 | 2.421e−27 | 361-382 | PR00766 | AMILORIDE-SENSITIVE |
| | | | | | | AMINE OXIDASE SIGNATURE |
| 1002 | PR00766F | 15.10 | 4.522e−28 | 439-462 | PR00766 | AMILORIDE-SENSITIVE |
| | | | | | | AMINE OXIDASE SIGNATURE |
| 1002 | PR00766G | 11.62 | 1.643e−27 | 678-698 | PR00766 | AMILORIDE-SENSITIVE |
| | | | | | | AMINE OXIDASE SIGNATURE |
| 1004 | PF00642 | 11.59 | 3.700e−16 | 166-176 | PF00642 | Zinc finger C-x8-C-x5- |
| | | | | | | C-x3-H type (and |
| | | | | | | similar). |
| 1004 | PF00642 | 11.59 | 7.900e−12 | 128-138 | PF00642 | Zinc finger C-x8-C-x5- |
| | | | | | | C-x3-H type (and |
| | | | | | | similar). |
| 1007 | BL00142 | 8.38 | 4.857e−09 | 582-592 | BL00142 | Neutral zinc |
| | | | | | | metallopeptidases, |
| | | | | | | zinc-binding region |
| | | | | | | proteins. |
| 1007 | PR00786A | 11.12 | 4.857e−13 | 536-548 | PR00786 | NEPRILYSIN |
| | | | | | | METALLOPROTEASE (M13) |
| | | | | | | FAMILY SIGNATURE |
| 1007 | PR00786B | 9.59 | 8.000e−17 | 554-566 | PR00786 | NEPRILYSIN |
| | | | | | | METALLOPROTEASE (M13) |
| | | | | | | FAMILY SIGNATURE |
| 1007 | PR00786C | 9.76 | 2.000e−22 | 575-591 | PR00786 | NEPRILYSIN |
| | | | | | | METALLOPROTEASE (M13) |
| | | | | | | FAMILY SIGNATURE |
| 1007 | PR00786D | 9.78 | 5.500e−14 | 648-659 | PR00786 | NEPRILYSIN |
| | | | | | | METALLOPROTEASE (M13) |
| | | | | | | FAMILY SIGNATURE |
| 1008 | BL00232A | 27.72 | 3.739e−26 | 82-114 | BL00232 | Cadherins extracellular |
| | | | | | | repeat proteins domain |
| | | | | | | proteins. |
| 1008 | BL00232B | 32.79 | 5.125e−40 | 174-221 | BL00232 | Cadherins extracellular |
| | | | | | | repeat proteins domain |
| | | | | | | proteins. |
| 1008 | BL00232B | 32.79 | 9.286e−19 | 281-328 | BL00232 | Cadherins extracellular |
| | | | | | | repeat proteins domain |
| | | | | | | proteins. |
| 1008 | BL00232C | 10.65 | 6.143e−16 | 279-296 | BL00232 | Cadherins extracellular |
| | | | | | | repeat proteins domain |
| | | | | | | proteins. |
| 1008 | BL00232D | 16.25 | 3.842e−13 | 796-810 | BL00232 | Cadherins extracellular |
| | | | | | | repeat proteins domain |
| | | | | | | proteins. |
| 1008 | PR00205A | 14.73 | 4.000e−12 | 206-221 | PR00205 | CADHERIN SIGNATURE |
| 1008 | PR00205B | 11.39 | 2.241e−16 | 279-296 | PR00205 | CADHERIN SIGNATURE |
| 1008 | PR00205C | 13.65 | 2.688e−10 | 540-554 | PR00205 | CADHERIN SIGNATURE |
| 1009 | BL00262A | 12.48 | 3.829e−09 | 57-74 | BL00262 | Insulin family |
| | | | | | | proteins. |
| 1009 | BL00262B | 16.89 | 4.977e−11 | 146-165 | BL00262 | Insulin family |
| | | | | | | proteins. |
| 1010 | PF00013 | 5.78 | 4.150e−09 | 127-138 | PF00013 | KH domain proteins |
| | | | | | | family of RNA binding |
| | | | | | | proteins. |
| 1011 | BL00719A | 16.66 | 7.488e−09 | 88-101 | BL00719 | Glycosyl hydrolases |
| | | | | | | family 2 proteins. |
| 1013 | BL00412D | 16.54 | 6.969e−09 | 75-125 | BL00412 | Neuromodulin (GAP-43) |
| | | | | | | proteins. |
| 1014 | PR00449D | 10.79 | 8.364e−12 | 55-68 | PR00449 | TRANSFORMING PROTEIN |
| | | | | | | P21 RAS SIGNATURE |
| 1014 | PR00449E | 13.50 | 4.706e−11 | 90-112 | PR00449 | TRANSFORMING PROTEIN |
| | | | | | | P21 RAS SIGNATURE |
| 1015 | PD02870B | 18.83 | 3.872e−09 | 345-377 | PD02870 | RECEPTOR INTERLEUKIN-1 |
| | | | | | | PRECURSOR. |
| 1016 | BL00022B | 7.54 | 1.000e−08 | 328-334 | BL00022 | EGF-like domain |
| | | | | | | proteins. |
| 1016 | BL01177C | 17.39 | 3.286e−09 | 394-412 | BL01177 | Anaphylatoxin domain |
| | | | | | | proteins. |
| 1016 | BL01177C | 17.39 | 5.119e−10 | 352-370 | BL01177 | Anaphylatoxin domain |
| | | | | | | proteins. |
| 1016 | BL01187A | 9.98 | 2.286e−13 | 343-354 | BL01187 | Calcium-binding EGF- |
| | | | | | | like domain proteins |
| | | | | | | pattern proteins. |
| 1016 | BL01187A | 9.98 | 4.750e−09 | 383-394 | BL01187 | Calcium-binding EGF- |
| | | | | | | like domain proteins |
| | | | | | | pattern proteins. |
| 1016 | BL01187B | 12.04 | 1.900e−09 | 276-291 | BL01187 | Calcium-binding EGF- |
| | | | | | | like domain proteins |
| | | | | | | pattern proteins. |
| 1016 | BL01187B | 12.04 | 3.667e−12 | 358-373 | BL01187 | Calcium-binding EGF- |
| | | | | | | like domain proteins |
| | | | | | | pattern proteins. |
| 1016 | BL01187B | 12.04 | 8.200e−14 | 443-458 | BL01187 | Calcium-binding EGF- |
| | | | | | | like domain proteins |
| | | | | | | pattern proteins. |
| 1016 | BL01187B | 12.04 | 8.826e−13 | 400-415 | BL01187 | Calcium-binding EGF- |
| | | | | | | like domain proteins |
| | | | | | | pattern proteins. |
| 1016 | PD00919A | 11.53 | 5.227e−09 | 306-317 | PD00919 | CALCIUM-BINDING |
| | | | | | | PRECURSOR SIGNAL R. |
| 1016 | PR00255E | 15.23 | 9.882e−09 | 586-608 | PR00255 | NATRIURETIC PEPTIDE |
| | | | | | | RECEPTOR SIGNATURE |
| 1016 | PR00352B | 13.51 | 6.400e−09 | 240-250 | PR00352 | 3FE-4S FERREDOXIN |
| | | | | | | SIGNATURE |
| 1016 | PR00907G | 11.63 | 1.243e−09 | 400-426 | PR00907 | THROMBOMODULIN |
| | | | | | | SIGNATURE |
| 1020 | BL00222B | 11.09 | 1.878e−12 | 114-129 | BL00222 | Insulin-like growth |
| | | | | | | factor binding |
| | | | | | | proteins. |
| 1020 | BL00222B | 11.09 | 3.250e−16 | 100-115 | BL00222 | Insulin-like growth |
| | | | | | | factor binding |
| | | | | | | proteins. |
| 1020 | BL00222C | 22.97 | 7.900e−25 | 158-185 | BL00222 | Insulin-like growth |
| | | | | | | factor binding |
| | | | | | | proteins. |
| 1020 | BL01185A | 8.11 | 6.318e−16 | 100-111 | BL01185 | C-terminal cystine knot |
| | | | | | | proteins. |
| 1020 | BL01185B | 21.14 | 1.000e−40 | 151-199 | BL01185 | C-terminal cystine knot |
| | | | | | | proteins. |
| 1020 | BL01185C | 15.86 | 2.038e−37 | 247-285 | BL01185 | C-terminal cystine knot |
| | | | | | | proteins. |
| 1020 | BL01185D | 23.45 | 1.000e−40 | 321-373 | BL01185 | C-terminal cystine knot |
| | | | | | | proteins. |
| 1020 | BL01208B | 15.83 | 5.500e−12 | 247-261 | BL01208 | VWFC domain proteins. |
| 1020 | BL01208C | 14.16 | 6.400e−11 | 280-290 | BL01208 | VWFC domain proteins. |
| 1021 | BL00030A | 14.39 | 9.571e−09 | 9-27 | BL00030 | Eukaryotic RNA-binding |
| | | | | | | region RNP-1 proteins. |
| 1021 | BL00030B | 7.03 | 1.000e−12 | 40-49 | BL00030 | Eukaryotic RNA-binding |
| | | | | | | region RNP-1 proteins. |
| 1021 | BL00048 | 6.39 | 1.000e−12 | 294-320 | BL00048 | Protamine P1 proteins. |
| 1021 | BL00048 | 6.39 | 2.059e−12 | 293-319 | BL00048 | Protamine P1 proteins. |
| 1021 | BL00048 | 6.39 | 2.324e−12 | 295-321 | BL00048 | Protamine P1 proteins. |
| 1021 | BL00048 | 6.39 | 2.913e−09 | 308-334 | BL00048 | Protamine P1 proteins. |
| 1021 | BL00048 | 6.39 | 2.938e−13 | 296-322 | BL00048 | Protamine P1 proteins. |
| 1021 | BL00048 | 6.39 | 3.125e−11 | 284-310 | BL00048 | Protamine P1 proteins. |
| 1021 | BL00048 | 6.39 | 3.250e−11 | 291-317 | BL00048 | Protamine P1 proteins. |
| 1021 | BL00048 | 6.39 | 3.625e−11 | 282-308 | BL00048 | Protamine P1 proteins. |
| 1021 | BL00048 | 6.39 | 3.700e−09 | 275-301 | BL00048 | Protamine P1 proteins. |
| 1021 | BL00048 | 6.39 | 3.724e−10 | 306-332 | BL00048 | Protamine P1 proteins. |
| 1021 | BL00048 | 6.39 | 3.750e−11 | 298-324 | BL00048 | Protamine P1 proteins. |
| 1021 | BL00048 | 6.39 | 4.046e−13 | 283-309 | BL00048 | Protamine P1 proteins. |
| 1021 | BL00048 | 6.39 | 4.176e−12 | 304-330 | BL00048 | Protamine P1 proteins. |
| 1021 | BL00048 | 6.39 | 4.671e−10 | 292-318 | BL00048 | Protamine P1 proteins. |
| 1021 | BL00048 | 6.39 | 5.015e−13 | 281-307 | BL00048 | Protamine P1 proteins. |
| 1021 | BL00048 | 6.39 | 5.125e−11 | 302-328 | BL00048 | Protamine P1 proteins. |
| 1021 | BL00048 | 6.39 | 5.145e−10 | 337-363 | BL00048 | Protamine P1 proteins. |
| 1021 | BL00048 | 6.39 | 5.500e−10 | 278-304 | BL00048 | Protamine P1 proteins. |
| 1021 | BL00048 | 6.39 | 5.846e−13 | 285-311 | BL00048 | Protamine P1 proteins. |
| 1021 | BL00048 | 6.39 | 5.974e−10 | 276-302 | BL00048 | Protamine P1 proteins. |
| 1021 | BL00048 | 6.39 | 6.092e−10 | 305-331 | BL00048 | Protamine P1 proteins. |
| 1021 | BL00048 | 6.39 | 6.329e−10 | 273-299 | BL00048 | Protamine P1 proteins. |
| 1021 | BL00048 | 6.39 | 6.400e−09 | 303-329 | BL00048 | Protamine P1 proteins. |
| 1021 | BL00048 | 6.39 | 7.492e−14 | 289-315 | BL00048 | Protamine P1 proteins. |
| 1021 | BL00048 | 6.39 | 7.500e−11 | 301-327 | BL00048 | Protamine P1 proteins. |
| 1021 | BL00048 | 6.39 | 7.785e−13 | 279-305 | BL00048 | Protamine P1 proteins. |
| 1021 | BL00048 | 6.39 | 8.377e−14 | 297-323 | BL00048 | Protamine P1 proteins. |
| 1021 | BL00048 | 6.39 | 8.538e−09 | 339-365 | BL00048 | Protamine P1 proteins. |
| 1021 | BL00048 | 6.39 | 8.627e−15 | 277-303 | BL00048 | Protamine P1 proteins. |
| 1021 | BL00048 | 6.39 | 8.672e−14 | 287-313 | BL00048 | Protamine P1 proteins. |
| 1021 | BL00048 | 6.39 | 8.816e−10 | 280-306 | BL00048 | Protamine P1 proteins. |
| 1021 | BL00048 | 6.39 | 8.941e−12 | 286-312 | BL00048 | Protamine P1 proteins. |
| 1021 | BL00048 | 6.39 | 9.000e−11 | 299-325 | BL00048 | Protamine P1 proteins. |
| 1021 | BL00048 | 6.39 | 9.100e−09 | 312-338 | BL00048 | Protamine P1 proteins. |
| 1021 | BL00048 | 6.39 | 9.308e−13 | 300-326 | BL00048 | Protamine P1 proteins. |
| 1021 | BL00048 | 6.39 | 9.723e−13 | 290-316 | BL00048 | Protamine P1 proteins. |
| 1021 | BL00048 | 6.39 | 9.735e−12 | 288-314 | BL00048 | Protamine P1 proteins. |
| 1021 | DM01206B | 10.69 | 1.000e−08 | 291-310 | DM01206 | CORONAVIRUS |
| | | | | | | NUCLEOCAPSID PROTEIN. |
| 1021 | DM01206B | 10.69 | 1.134e−11 | 287-306 | DM01206 | CORONAVIRUS |
| | | | | | | NUCLEOCAPSID PROTEIN. |
| 1021 | DM01206B | 10.69 | 3.284e−11 | 285-304 | DM01206 | CORONAVIRUS |
| | | | | | | NUCLEOCAPSID PROTEIN. |
| 1021 | DM01206B | 10.69 | 3.734e−09 | 310-329 | DM01206 | CORONAVIRUS |
| | | | | | | NUCLEOCAPSID PROTEIN. |
| 1021 | DM01206B | 10.69 | 4.714e−12 | 289-308 | DM01206 | CORONAVIRUS |
| | | | | | | NUCLEOCAPSID PROTEIN. |
| 1021 | DM01206B | 10.69 | 5.433e−11 | 283-302 | DM01206 | CORONAVIRUS |
| | | | | | | NUCLEOCAPSID PROTEIN. |
| 1021 | DM01206B | 10.69 | 5.438e−10 | 281-300 | DM01206 | CORONAVIRUS |
| | | | | | | NUCLEOCAPSID PROTEIN. |
| 1021 | DM01206B | 10.69 | 5.671e−09 | 346-365 | DM01206 | CORONAVIRUS |
| | | | | | | NUCLEOCAPSID PROTEIN. |
| 1021 | DM01206B | 10.69 | 5.970e−11 | 304-323 | DM01206 | CORONAVIRUS |
| | | | | | | NUCLEOCAPSID PROTEIN. |
| 1021 | DM01206B | 10.69 | 6.918e−10 | 306-325 | DM01206 | CORONAVIRUS |
| | | | | | | NUCLEOCAPSID PROTEIN. |
| 1021 | DM01206B | 10.69 | 7.714e−12 | 279-298 | DM01206 | CORONAVIRUS |
| | | | | | | NUCLEOCAPSID PROTEIN. |
| 1021 | DM01206B | 10.69 | 7.835e−09 | 293-312 | DM01206 | CORONAVIRUS |
| | | | | | | NUCLEOCAPSID PROTEIN. |
| 1021 | DM01206B | 10.69 | 7.949e−09 | 302-321 | DM01206 | CORONAVIRUS |
| | | | | | | NUCLEOCAPSID PROTEIN. |
| 1021 | DM01206B | 10.69 | 8.105e−13 | 308-327 | DM01206 | CORONAVIRUS |
| | | | | | | NUCLEOCAPSID PROTEIN. |
| 1021 | DM01206B | 10.69 | 8.105e−13 | 312-331 | DM01206 | CORONAVIRUS |
| | | | | | | NUCLEOCAPSID PROTEIN. |
| 1021 | DM01206B | 10.69 | 8.291e−09 | 277-296 | DM01206 | CORONAVIRUS |
| | | | | | | NUCLEOCAPSID PROTEIN. |
| 1022 | BL00345A | 13.96 | 3.903e−14 | 406-424 | BL00345 | Ets-domain proteins. |
| 1022 | BL00345B | 21.28 | 1.000e−40 | 439-489 | BL00345 | Ets-domain proteins. |
| 1022 | PD01234B | 15.53 | 3.893e−09 | 507-524 | PD01234 | PROTEIN NUCLEAR |
| | | | | | | BROMODOMAIN TRANS. |
| 1022 | PR00454A | 13.64 | 6.625e−13 | 410-423 | PR00454 | ETS DOMAIN SIGNATURE |
| 1022 | PR00454B | 20.78 | 3.625e−18 | 434-452 | PR00454 | ETS DOMAIN SIGNATURE |
| 1022 | PR00454C | 11.24 | 5.065e−22 | 453-471 | PR00454 | ETS DOMAIN SIGNATURE |
| 1022 | PR00454D | 10.89 | 5.950e−19 | 472-490 | PR00454 | ETS DOMAIN SIGNATURE |
| 1023 | BL00511 | 18.40 | 1.429e−31 | 193-224 | BL00511 | Corticotropin-releasing |
| | | | | | | factor family proteins. |
| 1027 | BL00456A | 22.59 | 8.527e−09 | 87-141 | BL00456 | Sodium: solute symporter |
| | | | | | | family proteins. |
| 1027 | BL00456C | 24.55 | 4.000e−22 | 223-277 | BL00456 | Sodium: solute symporter |
| | | | | | | family proteins. |
| 1029 | BL00360A | 16.97 | 6.478e−23 | 19-45 | BL00360 | Ribosomal protein S9 |
| | | | | | | proteins. |
| 1029 | BL00360B | 20.22 | 5.286e−29 | 62-97 | BL00360 | Ribosomal protein S9 |
| | | | | | | proteins. |
| 1029 | BL00360C | 17.65 | 8.105e−27 | 125-151 | BL00360 | Ribosomal protein S9 |
| | | | | | | proteins. |
| 1031 | BL00406A | 9.95 | 1.000e−40 | 31-65 | BL00406 | Actins proteins. |
| 1031 | BL00406B | 5.47 | 1.000e−40 | 107-161 | BL00406 | Actins proteins. |
| 1031 | BL00406C | 6.75 | 1.000e−40 | 166-220 | BL00406 | Actins proteins. |
| 1031 | BL00406D | 12.58 | 1.000e−40 | 293-347 | BL00406 | Actins proteins. |
| 1031 | BL00406E | 8.44 | 1.000e−40 | 350-399 | BL00406 | Actins proteins. |
| 1031 | PR00190A | 7.24 | 1.375e−13 | 50-59 | PR00190 | ACTIN SIGNATURE |
| 1031 | PR00190B | 9.98 | 1.000e−15 | 73-84 | PR00190 | ACTIN SIGNATURE |
| 1031 | PR00190C | 11.49 | 1.474e−30 | 85-107 | PR00190 | ACTIN SIGNATURE |
| 1031 | PR00190D | 19.23 | 1.450e−26 | 108-126 | PR00190 | ACTIN SIGNATURE |
| 1031 | PR00190E | 7.16 | 1.450e−19 | 139-152 | PR00190 | ACTIN SIGNATURE |
| 1031 | PR00190F | 7.80 | 2.286e−24 | 164-183 | PR00190 | ACTIN SIGNATURE |
| 1031 | PR00190G | 12.62 | 1.600e−21 | 260-276 | PR00190 | ACTIN SIGNATURE |
| 1032 | BL00180A | 13.20 | 5.065e−14 | 199-211 | BL00180 | Glutamine synthetase |
| | | | | | | proteins. |
| 1032 | BL00180B | 18.03 | 4.971e−14 | 235-253 | BL00180 | Glutamine synthetase |
| | | | | | | proteins. |
| 1032 | BL00180C | 12.14 | 4.600e−14 | 269-278 | BL00180 | Glutamine synthetase |
| | | | | | | proteins. |
| 1032 | BL00180D | 13.26 | 2.174e−24 | 286-307 | BL00180 | Glutamine synthetase |
| | | | | | | proteins. |
| 1032 | BL00180E | 17.60 | 1.000e−40 | 321-372 | BL00180 | Glutamine synthetase |
| | | | | | | proteins. |
| 1032 | BL00180F | 10.05 | 6.211e−17 | 385-397 | BL00180 | Glutamine synthetase |
| | | | | | | proteins. |
| 1032 | BL00180G | 10.20 | 8.435e−17 | 474-488 | BL00180 | Glutamine synthetase |
| | | | | | | proteins. |
| 1034 | BL00021B | 13.33 | 8.984e−12 | 545-562 | BL00021 | Kringle domain |
| | | | | | | proteins. |
| 1034 | BL00134A | 11.96 | 5.781e−15 | 545-561 | BL00134 | Serine proteases, |
| | | | | | | trypsin family, |
| | | | | | | histidine proteins. |
| 1034 | BL00134B | 15.99 | 4.194e−14 | 727-750 | BL00134 | Serine proteases, |
| | | | | | | trypsin family, |
| | | | | | | histidine proteins. |
| 1034 | BL00495N | 11.04 | 9.735e−11 | 719-753 | BL00495 | Apple domain proteins. |
| 1034 | BL01253G | 11.34 | 5.348e−09 | 726-739 | BL01253 | Type I fibronectin |
| | | | | | | domain proteins. |
| 1034 | PR00453A | 12.79 | 5.345e−14 | 303-320 | PR00453 | VON WILLEBRAND FACTOR |
| | | | | | | TYPE A DOMAIN SIGNATURE |
| 1034 | PR00453B | 14.65 | 4.682e−10 | 342-356 | PR00453 | VON WILLEBRAND FACTOR |
| | | | | | | TYPE A DOMAIN SIGNATURE |
| 1034 | PR00722A | 12.27 | 9.129e−13 | 546-561 | PR00722 | CHYMOTRYPSIN SERINE |
| | | | | | | PROTEASE FAMILY (S1) |
| | | | | | | SIGNATURE |
| 1034 | PR00722B | 12.51 | 4.000e−09 | 606-620 | PR00722 | CHYMOTRYPSIN SERINE |
| | | | | | | PROTEASE FAMILY (S1) |
| | | | | | | SIGNATURE |
| 1034 | PR00722C | 10.87 | 4.273e−11 | 726-738 | PR00722 | CHYMOTRYPSIN SERINE |
| | | | | | | PROTEASE FAMILY (S1) |
| | | | | | | SIGNATURE |
| 1036 | BL00018 | 7.41 | 7.300e−10 | 219-231 | BL00018 | EF-hand calcium-binding |
| | | | | | | domain proteins. |
| 1037 | BL00036 | 9.02 | 6.786e−13 | 86-98 | BL00036 | bZIP transcription |
| | | | | | | factors basic domain |
| | | | | | | proteins. |
| 1037 | PR00041C | 16.23 | 8.875e−16 | 36-47 | PR00041 | CAMP RESPONSE ELEMENT |
| | | | | | | BINDING (CREB) PROTEIN |
| | | | | | | SIGNATURE |
| 1037 | PR00041D | 7.95 | 4.750e−21 | 74-90 | PR00041 | CAMP RESPONSE ELEMENT |
| | | | | | | BINDING (CREB) PROTEIN |
| | | | | | | SIGNATURE |
| 1037 | PR00041E | 7.20 | 1.243e−27 | 92-112 | PR00041 | CAMP RESPONSE ELEMENT |
| | | | | | | BINDING (CREB) PROTEIN |
| | | | | | | SIGNATURE |
| 1037 | PR00041F | 8.53 | 2.125e−22 | 112-129 | PR00041 | CAMP RESPONSE ELEMENT |
| | | | | | | BINDING (CREB) PROTEIN |
| | | | | | | SIGNATURE |
| 1038 | PD00210 | 15.25 | 7.231e−16 | 47-62 | PD00210 | PROTEIN ANTIOXIDANT |
| | | | | | | PEROXIDASE RED. |
| 1039 | BL00028 | 16.07 | 6.143e−09 | 440-456 | BL00028 | Zinc finger, C2H2 type, |
| | | | | | | domain proteins. |
| 1039 | BL00028 | 16.07 | 8.826e−12 | 382-398 | BL00028 | Zinc finger, C2H2 type, |
| | | | | | | domain proteins. |
| 1039 | PD00066 | 13.92 | 6.870e−11 | 398-410 | PD00066 | PROTEIN ZINC-FINGER |
| | | | | | | METAL-BINDI. |
| 1039 | PR00048A | 10.52 | 9.280e−09 | 379-392 | PR00048 | C2H2-TYPE ZINC FINGER |
| | | | | | | SIGNATURE |
| 1039 | PR00308C | 3.83 | 8.714e−09 | 66-75 | PR00308 | TYPE I ANTIFREEZE |
| | | | | | | PROTEIN SIGNATURE |
| 1039 | PR00558C | 6.54 | 5.846e−09 | 37-56 | PR00558 | ALPHA-2A ADRENERGIC |
| | | | | | | RECEPTOR SIGNATURE |
| 1040 | BL00142 | 8.38 | 2.688e−10 | 351-361 | BL00142 | Neutral zinc |
| | | | | | | metallopeptidases, |
| | | | | | | zinc-binding region |
| | | | | | | proteins. |
| 1040 | BL00427 | 13.93 | 8.375e−25 | 454-508 | BL00427 | Disintegrins proteins. |
| 1040 | PR00289A | 13.62 | 7.525e−18 | 468-487 | PR00289 | DISINTEGRIN SIGNATURE |
| 1040 | PR00289B | 11.79 | 5.245e−10 | 497-509 | PR00289 | DISINTEGRIN SIGNATURE |
| 1040 | PR00451A | 6.49 | 5.696e−10 | 466-474 | PR00451 | CHITIN-BINDING DOMAIN |
| | | | | | | SIGNATURE |
| 1041 | BL01271A | 8.06 | 5.333e−21 | 147-166 | BL01271 | Sodium: sulfate |
| | | | | | | symporter family |
| | | | | | | proteins. |
| 1041 | BL01271A | 8.06 | 8.986e−09 | 487-506 | BL01271 | Sodium: sulfate |
| | | | | | | symporter family |
| | | | | | | proteins. |
| 1041 | BL01271B | 12.02 | 4.375e−19 | 246-270 | BL01271 | Sodium: sulfate |
| | | | | | | symporter family |
| | | | | | | proteins. |
| 1041 | BL01271C | 13.62 | 8.714e−20 | 438-459 | BL01271 | Sodium: sulfate |
| | | | | | | symporter family |
| | | | | | | proteins. |
| 1041 | BL01271D | 25.26 | 2.532e−32 | 511-565 | BL01271 | Sodium: sulfate |
| | | | | | | symporter family |
| | | | | | | proteins. |
| 1042 | PR00371E | 14.06 | 1.000e−10 | 66-75 | PR00371 | FLAVOPROTEIN PYRIDINE |
| | | | | | | NUCLEOTIDE CYTOCHROME |
| | | | | | | REDUCTASE SIGNATURE |
| 1042 | PR00371F | 10.12 | 5.135e−09 | 79-90 | PR00371 | FLAVOPROTEIN PYRIDINE |
| | | | | | | NUCLEOTIDE CYTOCHROME |
| | | | | | | REDUCTASE SIGNATURE |
| 1043 | BL00021B | 13.33 | 1.265e−20 | 120-137 | BL00021 | Kringle domain |
| | | | | | | proteins. |
| 1043 | BL00021C | 22.21 | 9.250e−10 | 203-224 | BL00021 | Kringle domain |
| | | | | | | proteins. |
| 1043 | BL00021D | 24.56 | 9.813e−25 | 290-331 | BL00021 | Kringle domain |
| | | | | | | proteins. |
| 1043 | BL00134A | 11.96 | 5.737e−19 | 120-136 | BL00134 | Serine proteases, |
| | | | | | | trypsin family, |
| | | | | | | histidine proteins. |
| 1043 | BL00134B | 15.99 | 7.231e−16 | 282-305 | BL00134 | Serine proteases, |
| | | | | | | trypsin family, |
| | | | | | | histidine proteins. |
| 1043 | BL00134C | 13.45 | 1.692e−11 | 318-331 | BL00134 | Serine proteases, |
| | | | | | | trypsin family, |
| | | | | | | histidine proteins. |
| 1043 | BL00495K | 12.58 | 8.600e−12 | 122-154 | BL00495 | Apple domain proteins. |
| 1043 | BL00495L | 11.94 | 6.757e−10 | 155-193 | BL00495 | Apple domain proteins. |
| 1043 | BL00495M | 8.50 | 4.058e−10 | 194-228 | BL00495 | Apple domain proteins. |
| 1043 | BL00495N | 11.04 | 1.409e−27 | 274-308 | BL00495 | Apple domain proteins. |
| 1043 | BL00495O | 13.75 | 7.459e−13 | 309-337 | BL00495 | Apple domain proteins. |
| 1043 | BL01253D | 4.84 | 5.500e−12 | 120-133 | BL01253 | Type I fibronectin |
| | | | | | | domain proteins. |
| 1043 | BL01253E | 16.01 | 8.579e−15 | 192-228 | BL01253 | Type I fibronectin |
| | | | | | | domain proteins. |
| 1043 | BL01253G | 11.34 | 1.000e−15 | 281-294 | BL01253 | Type I fibronectin |
| | | | | | | domain proteins. |
| 1043 | BL01253H | 13.15 | 5.500e−20 | 300-334 | BL01253 | Type I fibronectin |
| | | | | | | domain proteins. |
| 1043 | PR00722A | 12.27 | 8.941e−18 | 121-136 | PR00722 | CHYMOTRYPSIN SERINE |
| | | | | | | PROTEASE FAMILY (S1) |
| | | | | | | SIGNATURE |
| 1043 | PR00722B | 12.51 | 4.000e−09 | 180-194 | PR00722 | CHYMOTRYPSIN SERINE |
| | | | | | | PROTEASE FAMILY (S1) |
| | | | | | | SIGNATURE |
| 1043 | PR00722C | 10.87 | 5.235e−13 | 281-293 | PR00722 | CHYMOTRYPSIN SERINE |
| | | | | | | PROTEASE FAMILY (S1) |
| | | | | | | SIGNATURE |
| 1045 | BL00022B | 7.54 | 8.200e−09 | 865-871 | BL00022 | EGF-like domain |
| | | | | | | proteins. |
| 1045 | BL00279E | 37.11 | 9.241e−10 | 66-113 | BL00279 | Membrane attack complex |
| | | | | | | components/perforin |
| | | | | | | proteins. |
| 1045 | BL01187A | 9.98 | 1.000e−08 | 715-726 | BL01187 | Calcium-binding EGF- |
| | | | | | | like domain proteins |
| | | | | | | pattern proteins. |
| 1045 | BL01187A | 9.98 | 2.875e−09 | 921-932 | BL01187 | Calcium-binding EGF- |
| | | | | | | like domain proteins |
| | | | | | | pattern proteins. |
| 1045 | BL01187A | 9.98 | 7.000e−09 | 592-603 | BL01187 | Calcium-binding EGF- |
| | | | | | | like domain proteins |
| | | | | | | pattern proteins. |
| 1045 | BL01187A | 9.98 | 7.857e−10 | 756-767 | BL01187 | Calcium-binding EGF- |
| | | | | | | like domain proteins |
| | | | | | | pattern proteins. |
| 1045 | BL01187A | 9.98 | 9.526e−11 | 675-686 | BL01187 | Calcium-binding EGF- |
| | | | | | | like domain proteins |
| | | | | | | pattern proteins. |
| 1045 | BL01187B | 12.04 | 1.257e−10 | 1161-1176 | BL01187 | Calcium-binding EGF- |
| | | | | | | like domain proteins |
| | | | | | | pattern proteins. |
| 1045 | BL01187B | 12.04 | 1.391e−13 | 321-336 | BL01187 | Calcium-binding EGF- |
| | | | | | | like domain proteins |
| | | | | | | pattern proteins. |
| 1045 | BL01187B | 12.04 | 2.350e−14 | 856-871 | BL01187 | Calcium-binding EGF- |
| | | | | | | like domain proteins |
| | | | | | | pattern proteins. |
| 1045 | BL01187B | 12.04 | 4.130e−13 | 897-912 | BL01187 | Calcium-binding EGF- |
| | | | | | | like domain proteins |
| | | | | | | pattern proteins. |
| 1045 | BL01187B | 12.04 | 4.600e−11 | 939-954 | BL01187 | Calcium-binding EGF- |
| | | | | | | like domain proteins |
| | | | | | | pattern proteins. |
| 1045 | BL01187B | 12.04 | 4.913e−13 | 773-788 | BL01187 | Calcium-binding EGF- |
| | | | | | | like domain proteins |
| | | | | | | pattern proteins. |
| 1045 | BL01187B | 12.04 | 5.500e−14 | 814-829 | BL01187 | Calcium-binding EGF- |
| | | | | | | like domain proteins |
| | | | | | | pattern proteins. |
| 1045 | BL01187B | 12.04 | 7.000e−17 | 1360-1375 | BL01187 | Calcium-binding EGF- |
| | | | | | | like domain proteins |
| | | | | | | pattern proteins. |
| 1045 | BL01187B | 12.04 | 9.229e−10 | 1315-1330 | BL01187 | Calcium-binding EGF- |
| | | | | | | like domain proteins |
| | | | | | | pattern proteins. |
| 1045 | BL01187B | 12.04 | 9.609e−13 | 610-625 | BL01187 | Calcium-binding EGF- |
| | | | | | | like domain proteins |
| | | | | | | pattern proteins. |
| 1045 | BL01187B | 12.04 | 9.667e−12 | 732-747 | BL01187 | Calcium-binding EGF- |
| | | | | | | like domain proteins |
| | | | | | | pattern proteins. |
| 1045 | PD00919B | 9.47 | 7.485e−10 | 697-711 | PD00919 | CALCIUM-BINDING |
| | | | | | | PRECURSOR SIGNAL R. |
| 1045 | PR00009C | 14.11 | 9.118e−09 | 736-747 | PR00009 | TYPE I EGF SIGNATURE |
| 1045 | PR00010C | 11.16 | 1.857e−09 | 615-625 | PR00010 | TYPE II EGF-LIKE |
| | | | | | | SIGNATURE |
| 1045 | PR00010C | 11.16 | 4.857e−09 | 1365-1375 | PR00010 | TYPE II EGF-LIKE |
| | | | | | | SIGNATURE |
| 1045 | PR00010C | 11.16 | 7.545e−10 | 1320-1330 | PR00010 | TYPE II EGF-LIKE |
| | | | | | | SIGNATURE |
| 1045 | PR00010C | 11.16 | 7.667e−11 | 861-871 | PR00010 | TYPE II EGF-LIKE |
| | | | | | | SIGNATURE |
| 1045 | PR00011D | 14.03 | 2.696e−09 | 92-110 | PR00011 | TYPE III EGF-LIKE |
| | | | | | | SIGNATURE |
| 1045 | PR00907G | 11.63 | 7.973e−09 | 568-594 | PR00907 | THROMBOMODULIN |
| | | | | | | SIGNATURE |
| 1048 | BL00352A | 12.19 | 7.000e−14 | 377-391 | BL00352 | ‘Cold-shock’ DNA- |
| | | | | | | binding domain |
| | | | | | | proteins. |
| 1048 | BL00352B | 23.66 | 2.000e−20 | 737-775 | BL00352 | ‘Cold-shock’ DNA- |
| | | | | | | binding domain |
| | | | | | | proteins. |
| 1050 | BL00326A | 14.01 | 6.128e−09 | 1284-1317 | BL00326 | Tropomyosins proteins. |
| 1050 | BL00447C | 13.79 | 9.813e−09 | 198-212 | BL00447 | DNA polymerase family A |
| | | | | | | proteins. |
| 1050 | DM00215 | 19.43 | 7.712e−09 | 1026-1058 | DM00215 | PROLINE-RICH PROTEIN 3. |
| 1050 | PD00301B | 5.49 | 6.885e−09 | 1095-1105 | PD00301 | PROTEIN REPEAT MUSCLE |
| | | | | | | CALCIUM-BI. |
| 1050 | PF00992A | 16.67 | 1.355e−09 | 1297-1331 | PF00992 | Troponin. |
| 1050 | PF00992A | 16.67 | 4.434e−09 | 1289-1323 | PF00992 | Troponin. |
| 1050 | PF00992A | 16.67 | 7.513e−09 | 1298-1332 | PF00992 | Troponin. |
| 1050 | PF01140D | 15.54 | 1.628e−09 | 1073-1107 | PF011040 | Matrix protein (MA), |
| | | | | | | p15. |
| 1050 | PF01140D | 15.54 | 4.872e−09 | 1295-1329 | PF01140 | Matrix protein (MA), |
| | | | | | | p15. |
| 1051 | BL00310A | 14.05 | 7.000e−15 | 61-75 | BL00310 | Lysosome-associated |
| | | | | | | membrane glycoproteins |
| | | | | | | duplicated domain |
| | | | | | | proteins. |
| 1051 | BL00310B | 22.33 | 5.034e−32 | 113-148 | BL00310 | Lysosome-associated |
| | | | | | | membrane glycoproteins |
| | | | | | | duplicated domain |
| | | | | | | proteins. |
| 1051 | BL00310C | 26.35 | 3.400e−39 | 166-212 | BL00310 | Lysosome-associated |
| | | | | | | membrane glycoproteins |
| | | | | | | duplicated domain |
| | | | | | | proteins. |
| 1051 | BL00310D | 14.97 | 7.818e−28 | 238-264 | BL00310 | Lysosome-associated |
| | | | | | | membrane glycoproteins |
| | | | | | | duplicated domain |
| | | | | | | proteins. |
| 1051 | BL00310E | 19.92 | 2.250e−24 | 339-363 | BL00310 | Lysosome-associated |
| | | | | | | membrane glycoproteins |
| | | | | | | duplicated domain |
| | | | | | | proteins. |
| 1051 | BL00310F | 23.26 | 1.000e−40 | 374-428 | BL00310 | Lysosome-associated |
| | | | | | | membrane glycoproteins |
| | | | | | | duplicated domain |
| | | | | | | proteins. |
| 1051 | PR00336A | 12.86 | 9.727e−24 | 241-265 | PR00336 | LYSOSOME-ASSOCIATED |
| | | | | | | MEMBRANE GLYCOPROTEIN |
| | | | | | | SIGNATURE |
| 1051 | PR00336B | 15.42 | 4.000e−16 | 351-365 | PR00336 | LYSOSOME-ASSOCIATED |
| | | | | | | MEMBRANE GLYCOPROTEIN |
| | | | | | | SIGNATURE |
| 1051 | PR00336C | 14.47 | 9.333e−12 | 388-400 | PR00336 | LYSOSOME-ASSOCIATED |
| | | | | | | MEMBRANE GLYCOPROTEIN |
| | | | | | | SIGNATURE |
| 1051 | PR00336D | 9.96 | 4.150e−24 | 402-424 | PR00336 | LYSOSOME-ASSOCIATED |
| | | | | | | MEMBRANE GLYCOPROTEIN |
| | | | | | | SIGNATURE |
| 1051 | PR00336E | 11.59 | 3.382e−13 | 425-437 | PR00336 | LYSOSOME-ASSOCIATED |
| | | | | | | MEMBRANE GLYCOPROTEIN |
| | | | | | | SIGNATURE |
| 1052 | PF00550B | 10.27 | 2.588e−10 | 129-142 | PF00550 | Phosphopantetheine |
| | | | | | | attachment site |
| | | | | | | proteins. |
| 1055 | BL00690A | 6.87 | 3.455e−12 | 424-433 | BL00690 | DEAH-box subfamily ATP- |
| | | | | | | dependent helicases |
| | | | | | | proteins. |
| 1055 | BL00690B | 13.38 | 6.571e−16 | 453-470 | BL00690 | DEAH-box subfamily ATP- |
| | | | | | | dependent helicases |
| | | | | | | proteins. |
| 1055 | BL00690C | 7.51 | 7.429e−10 | 519-528 | BL00690 | DEAH-box subfamily ATP- |
| | | | | | | dependent helicases |
| | | | | | | proteins. |
| 1056 | PR00704A | 14.68 | 7.375e−22 | 60-83 | PR00704 | CALPAIN CYSTEINE |
| | | | | | | PROTEASE (C2) FAMILY |
| | | | | | | SIGNATURE |
| 1056 | PR00704B | 17.94 | 6.276e−23 | 105-127 | PR00704 | CALPAIN CYSTEINE |
| | | | | | | PROTEASE (C2) FAMILY |
| | | | | | | SIGNATURE |
| 1056 | PR00704C | 11.88 | 6.625e−20 | 129-145 | PR00704 | CALPAIN CYSTEINE |
| | | | | | | PROTEASE (C2) FAMILY |
| | | | | | | SIGNATURE |
| 1056 | PR00704D | 11.05 | 5.065e−29 | 165-190 | PR00704 | CALPAIN CYSTEINE |
| | | | | | | PROTEASE (C2) FAMILY |
| | | | | | | SIGNATURE |
| 1056 | PR00704E | 12.55 | 8.875e−28 | 195-218 | PR00704 | CALPAIN CYSTEINE |
| | | | | | | PROTEASE (C2) FAMILY |
| | | | | | | SIGNATURE |
| 1056 | PR00704F | 13.61 | 7.632e−30 | 220-247 | PR00704 | CALPAIN CYSTEINE |
| | | | | | | PROTEASE (C2) FAMILY |
| | | | | | | SIGNATURE |
| 1056 | PR00704G | 13.87 | 1.391e−25 | 350-371 | PR00704 | CALPAIN CYSTEINE |
| | | | | | | PROTEASE (C2) FAMILY |
| | | | | | | SIGNATURE |
| 1056 | PR00704H | 13.38 | 9.217e−22 | 400-417 | PR00704 | CALPAIN CYSTEINE |
| | | | | | | PROTEASE (C2) FAMILY |
| | | | | | | SIGNATURE |
| 1056 | PR00704I | 9.52 | 1.000e−30 | 508-536 | PR00704 | CALPAIN CYSTEINE |
| | | | | | | PROTEASE (C2) FAMILY |
| | | | | | | SIGNATURE |
| 1057 | BL01108A | 20.33 | 2.241e−22 | 135-167 | BL01108 | Ribosomal protein L24 |
| | | | | | | proteins. |
| 1057 | BL01108B | 11.40 | 8.457e−10 | 182-192 | BL01108 | Ribosomal protein L24 |
| | | | | | | proteins. |
| 1058 | BL00250A | 21.24 | 4.130e−27 | 263-298 | BL00250 | TGF-beta family |
| | | | | | | proteins. |
| 1058 | BL00250B | 27.37 | 3.000e−20 | 331-366 | BL00250 | TGF-beta family |
| | | | | | | proteins. |
| 1058 | PR00669A | 12.84 | 4.414e−31 | 110-134 | PR00669 | INHIBIN ALPHA CHAIN |
| | | | | | | SIGNATURE |
| 1058 | PR00669B | 8.27 | 6.927e−20 | 188-204 | PR00669 | INHIBIN ALPHA CHAIN |
| | | | | | | SIGNATURE |
| 1058 | PR00669C | 14.90 | 1.220e−21 | 206-223 | PR00669 | INHIBIN ALPHA CHAIN |
| | | | | | | SIGNATURE |
| 1058 | PR00669D | 7.36 | 5.974e−25 | 241-260 | PR00669 | INHIBIN ALPHA CHAIN |
| | | | | | | SIGNATURE |
| 1058 | PR00669E | 16.28 | 3.152e−23 | 263-280 | PR00669 | INHIBIN ALPHA CHAIN |
| | | | | | | SIGNATURE |
| 1058 | PR00669F | 5.57 | 6.077e−25 | 280-297 | PR00669 | INHIBIN ALPHA CHAIN |
| | | | | | | SIGNATURE |
| 1058 | PR00669G | 7.46 | 8.650e−26 | 324-343 | PR00669 | INHIBIN ALPHA CHAIN |
| | | | | | | SIGNATURE |
| 1058 | PR00669H | 19.13 | 3.893e−29 | 345-366 | PR00669 | INHIBIN ALPHA CHAIN |
| | | | | | | SIGNATURE |
| 1058 | PR00712A | 6.07 | 7.722e−10 | 4-20 | PR00712 | BRAIN NATRIURETIC |
| | | | | | | PEPTIDE SIGNATURE |
| 1059 | PR00854B | 7.30 | 2.519e−10 | 5-22 | PR00854 | PROSTAGLANDIN D |
| | | | | | | RECEPTOR SIGNATURE |
| 1063 | PR00846A | 15.43 | 1.818e−13 | 49-61 | PR00846 | GLYCOSYL HYDROLASE |
| | | | | | | FAMILY 56 SIGNATURE |
| 1063 | PR00846B | 13.97 | 7.750e−17 | 109-123 | PR00846 | GLYCOSYL HYDROLASE |
| | | | | | | FAMILY 56 SIGNATURE |
| 1063 | PR00846C | 12.18 | 5.696e−23 | 141-158 | PR00846 | GLYCOSYL HYDROLASE |
| | | | | | | FAMILY 56 SIGNATURE |
| 1063 | PR00846D | 17.96 | 4.130e−29 | 199-224 | PR00846 | GLYCOSYL HYDROLASE |
| | | | | | | FAMILY 56 SIGNATURE |
| 1063 | PR00846E | 12.07 | 3.700e−17 | 248-262 | PR00846 | GLYCOSYL HYDROLASE |
| | | | | | | FAMILY 56 SIGNATURE |
| 1063 | PR00846F | 14.27 | 5.875e−13 | 353-366 | PR00846 | GLYCOSYL HYDROLASE |
| | | | | | | FAMILY 56 SIGNATURE |
| 1064 | BL00226A | 12.77 | 2.543e−12 | 97-111 | BL00226 | Intermediate filaments |
| | | | | | | proteins. |
| 1064 | BL00226B | 23.86 | 1.000e−31 | 197-244 | BL00226 | Intermediate filaments |
| | | | | | | proteins. |
| 1064 | BL00226B | 23.86 | 7.600e−11 | 148-195 | BL00226 | Intermediate filaments |
| | | | | | | proteins. |
| 1064 | BL00226C | 13.23 | 1.000e−22 | 263-293 | BL00226 | Intermediate filaments |
| | | | | | | proteins. |
| 1064 | BL00226D | 19.10 | 1.000e−40 | 364-410 | BL00226 | Intermediate filaments |
| | | | | | | proteins. |
| 1064 | PR00042D | 8.97 | 5.793e−09 | 325-346 | PR00042 | FOS TRANSFORMING |
| | | | | | | PROTEIN SIGNATURE |
| 1066 | BL00610B | 23.65 | 9.265e−09 | 38-87 | BL00610 | Sodium: neurotransmitter |
| | | | | | | symporter family |
| | | | | | | proteins. |
| 1067 | BL00965B | 17.77 | 9.735e−11 | 146-172 | BL00965 | Phosphomannose |
| | | | | | | isomerase type I |
| | | | | | | proteins. |
| 1067 | BL00965C | 23.78 | 1.225e−14 | 341-389 | BL00965 | Phosphomannose |
| | | | | | | isomerase type I |
| | | | | | | proteins. |
| 1067 | PF00801B | 23.63 | 6.344e−11 | 289-316 | PF00801 | PKD domain proteins. |
| 1067 | PR00049D | 0.00 | 5.689e−11 | 319-333 | PR00049 | WILM'S TUMOUR PROTEIN |
| | | | | | | SIGNATURE |
| 1067 | PR00049D | 0.00 | 6.643e−10 | 321-335 | PR00049 | WILM'S TUMOUR PROTEIN |
| | | | | | | SIGNATURE |
| 1067 | PR00049D | 0.00 | 9.786e−10 | 323-337 | PR00049 | WILM'S TUMOUR PROTEIN |
| | | | | | | SIGNATURE |
| 1067 | PR00910A | 2.51 | 8.393e−09 | 323-335 | PR00910 | LUTEOVIRUS ORF6 PROTEIN |
| | | | | | | SIGNATURE |
| 1069 | BL01019A | 13.20 | 9.520e−13 | 58-97 | BL01019 | ADP-ribosylation |
| | | | | | | factors family |
| | | | | | | proteins. |
| 1069 | BL01115A | 10.22 | 5.075e−11 | 24-67 | BL01115 | GTP-binding nuclear |
| | | | | | | protein ran proteins. |
| 1069 | PR00449A | 13.20 | 5.846e−21 | 24-45 | PR00449 | TRANSFORMING PROTEIN |
| | | | | | | P21 RAS SIGNATURE |
| 1069 | PR00449B | 14.34 | 5.500e−13 | 47-63 | PR00449 | TRANSFORMING PROTEIN |
| | | | | | | P21 RAS SIGNATURE |
| 1069 | PR00449C | 17.27 | 3.700e−24 | 65-87 | PR00449 | TRANSFORMING PROTEIN |
| | | | | | | P21 RAS SIGNATURE |
| 1069 | PR00449D | 10.79 | 4.273e−12 | 127-140 | PR00449 | TRANSFORMING PROTEIN |
| | | | | | | P21 RAS SIGNATURE |
| 1069 | PR00449E | 13.50 | 7.000e−19 | 162-184 | PR00449 | TRANSFORMING PROTEIN |
| | | | | | | P21 RAS SIGNATURE |
| 1070 | BL00415B | 9.91 | 9.404e−09 | 731-766 | BL00415 | Synapsins proteins. |
| 1070 | BL00415N | 4.29 | 7.832e−09 | 1057-1100 | BL00415 | Synapsins proteins. |
| 1070 | BL00904A | 8.30 | 6.489e−09 | 496-545 | BL00904 | Protein |
| | | | | | | prenyltransferases |
| | | | | | | alpha subunit repeat |
| | | | | | | proteins proteins. |
| 1070 | DM00215 | 19.43 | 3.746e−09 | 1043-1075 | DM00215 | PROLINE-RICH PROTEIN 3. |
| 1070 | PR00049D | 0.00 | 2.831e−09 | 1299-1313 | PR00049 | WILM'S TUMOUR PROTEIN |
| | | | | | | SIGNATURE |
| 1070 | PR00049D | 0.00 | 8.169e−09 | 1301-1315 | PR00049 | WILM'S TUMOUR PROTEIN |
| | | | | | | SIGNATURE |
| 1070 | PR00628A | 14.81 | 8.134e−19 | 233-252 | PR00628 | INSULIN RECEPTOR |
| | | | | | | SUBSTRATE-1 PTB DOMAIN |
| | | | | | | SIGNATURE |
| 1070 | PR00628B | 14.71 | 2.556e−15 | 254-274 | PR00628 | INSULIN RECEPTOR |
| | | | | | | SUBSTRATE-1 PTB DOMAIN |
| | | | | | | SIGNATURE |
| 1070 | PR00628C | 16.37 | 9.289e−16 | 276-292 | PR00628 | INSULIN RECEPTOR |
| | | | | | | SUBSTRATE-1 PTB DOMAIN |
| | | | | | | SIGNATURE |
| 1070 | PR00628D | 15.47 | 4.000e−15 | 293-305 | PR00628 | INSULIN RECEPTOR |
| | | | | | | SUBSTRATE-1 PTB DOMAIN |
| | | | | | | SIGNATURE |
| 1070 | PR00628F | 16.74 | 8.116e−18 | 317-341 | PR00628 | INSULIN RECEPTOR |
| | | | | | | SUBSTRATE-1 PTB DOMAIN |
| | | | | | | SIGNATURE |
| 1071 | BL00143A | 20.91 | 4.808e−22 | 116-142 | BL00143 | Insulinase family, |
| | | | | | | zinc-binding region |
| | | | | | | proteins. |
| 1071 | BL00143B | 14.41 | 3.077e−13 | 162-176 | BL00143 | Insulinase family, |
| | | | | | | zinc-binding region |
| | | | | | | proteins. |
| 1071 | BL00143C | 14.16 | 7.000e−11 | 264-276 | BL00143 | Insulinase family, |
| | | | | | | zinc-binding region |
| | | | | | | proteins. |
| 1072 | PF00686C | 12.33 | 8.740e−11 | 320-340 | PF00686 | Starch binding domain |
| | | | | | | proteins. |
| 1074 | DM00215 | 19.43 | 2.220e−09 | 41-73 | DM00215 | PROLINE-RICH PROTEIN 3. |
| 1074 | DM00215 | 19.43 | 3.593e−09 | 77-109 | DM00215 | PROLINE-RICH PROTEIN 3. |
| 1074 | DM00215 | 19.43 | 8.780e−09 | 84-116 | DM00215 | PROLINE-RICH PROTEIN 3. |
| 1074 | DM00215 | 19.43 | 9.847e−09 | 81-113 | DM00215 | PROLINE-RICH PROTEIN 3. |
| 1074 | PR00049D | 0.00 | 4.966e−09 | 77-91 | PR00049 | WILM'S TUMOUR PROTEIN |
| | | | | | | SIGNATURE |
| 1074 | PR00049D | 0.00 | 9.237e−09 | 101-115 | PR00049 | WILM'S TUMOUR PROTEIN |
| | | | | | | SIGNATURE |
| 1076 | BL00022B | 7.54 | 6.400e−09 | 672-678 | BL00022 | EGF-like domain |
| | | | | | | proteins. |
| 1076 | BL00243H | 17.53 | 9.526e−09 | 570-595 | BL00243 | Integrins beta chain |
| | | | | | | cysteine-rich domain |
| | | | | | | proteins. |
| 1076 | BL00243I | 31.77 | 4.549e−09 | 2113-2155 | BL00243 | Integrins beta chain |
| | | | | | | cysteine-rich domain |
| | | | | | | proteins. |
| 1076 | BL00790E | 29.58 | 7.964e−11 | 548-595 | BL00790 | Receptor tyrosine |
| | | | | | | kinase class V |
| | | | | | | proteins. |
| 1076 | BL01248 | 11.02 | 1.000e−11 | 346-358 | BL01248 | Laminin-type EGF-like |
| | | | | | | (LE) domain proteins. |
| 1076 | BL01248 | 11.02 | 2.895e−16 | 1905-1917 | BL01248 | Laminin-type EGF-like |
| | | | | | | (LE) domain proteins. |
| 1076 | BL01248 | 11.02 | 3.377e−09 | 539-551 | BL01248 | Laminin-type EGF-like |
| | | | | | | (LE) domain proteins. |
| 1076 | BL01248 | 11.02 | 5.415e−09 | 629-641 | BL01248 | Laminin-type EGF-like |
| | | | | | | (LE) domain proteins. |
| 1076 | BL01248 | 11.02 | 6.625e−10 | 2016-2028 | BL01248 | Laminin-type EGF-like |
| | | | | | | (LE) domain proteins. |
| 1076 | BL01248 | 11.02 | 7.000e−14 | 418-430 | BL01248 | Laminin-type EGF-like |
| | | | | | | (LE) domain proteins. |
| 1076 | BL01248 | 11.02 | 8.132e−09 | 2112-2124 | BL01248 | Laminin-type EGF-like |
| | | | | | | (LE) domain proteins. |
| 1076 | BL01248 | 11.02 | 8.302e−09 | 2146-2158 | BL01248 | Laminin-type EGF-like |
| | | | | | | (LE) domain proteins. |
| 1076 | BL01248 | 11.02 | 8.302e−09 | 575-587 | BL01248 | Laminin-type EGF-like |
| | | | | | | (LE) domain proteins. |
| 1076 | BL01248 | 11.02 | 9.491e−09 | 1574-1586 | BL01248 | Laminin-type EGF-like |
| | | | | | | (LE) domain proteins. |
| 1076 | PD00320A | 14.49 | 7.750e−09 | 352-365 | PD00320 | LAMININ CHAIN EGF-LIKE |
| | | | | | | DOMAIN P. |
| 1076 | PD00320A | 14.49 | 9.357e−09 | 827-840 | PD00320 | LAMININ CHAIN EGF-LIKE |
| | | | | | | DOMAIN P. |
| 1076 | PR00011A | 14.06 | 1.196e−17 | 1611-1629 | PR00011 | TYPE III EGF-LIKE |
| | | | | | | SIGNATURE |
| 1076 | PR00011A | 14.06 | 1.391e−10 | 575-593 | PR00011 | TYPE III EGF-LIKE |
| | | | | | | SIGNATURE |
| 1076 | PR00011A | 14.06 | 2.452e−12 | 462-480 | PR00011 | TYPE III EGF-LIKE |
| | | | | | | SIGNATURE |
| 1076 | PR00011A | 14.06 | 2.875e−16 | 711-729 | PR00011 | TYPE III EGF-LIKE |
| | | | | | | SIGNATURE |
| 1076 | PR00011A | 14.06 | 3.625e−16 | 1565-1583 | PR00011 | TYPE III EGF-LIKE |
| | | | | | | SIGNATURE |
| 1076 | PR00011A | 14.06 | 5.271e−13 | 863-881 | PR00011 | TYPE III EGF-LIKE |
| | | | | | | SIGNATURE |
| 1076 | PR00011A | 14.06 | 5.313e−16 | 812-830 | PR00011 | TYPE III EGF-LIKE |
| | | | | | | SIGNATURE |
| 1076 | PR00011A | 14.06 | 6.081e−12 | 2103-2121 | PR00011 | TYPE III EGF-LIKE |
| | | | | | | SIGNATURE |
| 1076 | PR00011A | 14.06 | 7.873e−14 | 1513-1531 | PR00011 | TYPE III EGF-LIKE |
| | | | | | | SIGNATURE |
| 1076 | PR00011A | 14.06 | 9.660e−15 | 1472-1490 | PR00011 | TYPE III EGF-LIKE |
| | | | | | | SIGNATURE |
| 1076 | PR00011A | 14.06 | 9.673e−14 | 2055-2073 | PR00011 | TYPE III EGF-LIKE |
| | | | | | | SIGNATURE |
| 1076 | PR00011A | 14.06 | 9.710e−12 | 530-548 | PR00011 | TYPE III EGF-LIKE |
| | | | | | | SIGNATURE |
| 1076 | PR00011A | 14.06 | 9.830e−15 | 2146-2164 | PR00011 | TYPE III EGF-LIKE |
| | | | | | | SIGNATURE |
| 1076 | PR00011B | 13.08 | 1.458e−13 | 1611-1629 | PR00011 | TYPE III EGF-LIKE |
| | | | | | | SIGNATURE |
| 1076 | PR00011B | 13.08 | 1.610e−13 | 812-830 | PR00011 | TYPE III EGF-LIKE |
| | | | | | | SIGNATURE |
| 1076 | PR00011B | 13.08 | 1.614e−17 | 2055-2073 | PR00011 | TYPE III EGF-LIKE |
| | | | | | | SIGNATURE |
| 1076 | PR00011B | 13.08 | 1.692e−15 | 575-593 | PR00011 | TYPE III EGF-LIKE |
| | | | | | | SIGNATURE |
| 1076 | PR00011B | 13.08 | 1.740e−09 | 530-548 | PR00011 | TYPE III EGF-LIKE |
| | | | | | | SIGNATURE |
| 1076 | PR00011B | 13.08 | 1.833e−14 | 2103-2121 | PR00011 | TYPE III EGF-LIKE |
| | | | | | | SIGNATURE |
| 1076 | PR00011B | 13.08 | 3.177e−12 | 462-480 | PR00011 | TYPE III EGF-LIKE |
| | | | | | | SIGNATURE |
| 1076 | PR00011B | 13.08 | 3.942e−15 | 1565-1583 | PR00011 | TYPE III EGF-LIKE |
| | | | | | | SIGNATURE |
| 1076 | PR00011B | 13.08 | 4.500e−14 | 2146-2164 | PR00011 | TYPE III EGF-LIKE |
| | | | | | | SIGNATURE |
| 1076 | PR00011B | 13.08 | 5.875e−16 | 711-729 | PR00011 | TYPE III EGF-LIKE |
| | | | | | | SIGNATURE |
| 1076 | PR00011B | 13.08 | 8.017e−13 | 1472-1490 | PR00011 | TYPE III EGF-LIKE |
| | | | | | | SIGNATURE |
| 1076 | PR00011B | 13.08 | 9.217e−10 | 1513-1531 | PR00011 | TYPE III EGF-LIKE |
| | | | | | | SIGNATURE |
| 1076 | PR00011B | 13.08 | 9.862e−11 | 863-881 | PR00011 | TYPE III EGF-LIKE |
| | | | | | | SIGNATURE |
| 1076 | PR00011C | 24.25 | 1.450e−10 | 1618-1646 | PR00011 | TYPE III EGF-LIKE |
| | | | | | | SIGNATURE |
| 1076 | PR00011C | 24.25 | 4.571e−09 | 766-794 | PR00011 | TYPE III EGF-LIKE |
| | | | | | | SIGNATURE |
| 1076 | PR00011C | 24.25 | 5.143e−09 | 2122-2150 | PR00011 | TYPE III EGF-LIKE |
| | | | | | | SIGNATURE |
| 1076 | PR00011C | 24.25 | 8.714e−09 | 546-574 | PR00011 | TYPE III EGF-LIKE |
| | | | | | | SIGNATURE |
| 1076 | PR00011D | 14.03 | 1.551e−14 | 462-480 | PR00011 | TYPE III EGF-LIKE |
| | | | | | | SIGNATURE |
| 1076 | PR00011D | 14.03 | 2.328e−11 | 1472-1490 | PR00011 | TYPE III EGF-LIKE |
| | | | | | | SIGNATURE |
| 1076 | PR00011D | 14.03 | 2.653e−14 | 2103-2121 | PR00011 | TYPE III EGF-LIKE |
| | | | | | | SIGNATURE |
| 1076 | PR00011D | 14.03 | 3.361e−11 | 530-548 | PR00011 | TYPE III EGF-LIKE |
| | | | | | | SIGNATURE |
| 1076 | PR00011D | 14.03 | 3.800e−15 | 1611-1629 | PR00011 | TYPE III EGF-LIKE |
| | | | | | | SIGNATURE |
| 1076 | PR00011D | 14.03 | 3.803e−11 | 863-881 | PR00011 | TYPE III EGF-LIKE |
| | | | | | | SIGNATURE |
| 1076 | PR00011D | 14.03 | 4.375e−16 | 2055-2073 | PR00011 | TYPE III EGF-LIKE |
| | | | | | | SIGNATURE |
| 1076 | PR00011D | 14.03 | 4.836e−11 | 812-830 | PR00011 | TYPE III EGF-LIKE |
| | | | | | | SIGNATURE |
| 1076 | PR00011D | 14.03 | 5.224e−14 | 575-593 | PR00011 | TYPE III EGF-LIKE |
| | | | | | | SIGNATURE |
| 1076 | PR00011D | 14.03 | 5.500e−17 | 1565-1583 | PR00011 | TYPE III EGF-LIKE |
| | | | | | | SIGNATURE |
| 1076 | PR00011D | 14.03 | 5.826e−09 | 1513-1531 | PR00011 | TYPE III EGF-LIKE |
| | | | | | | SIGNATURE |
| 1076 | PR00011D | 14.03 | 6.815e−10 | 665-683 | PR00011 | TYPE III EGF-LIKE |
| | | | | | | SIGNATURE |
| 1076 | PR00011D | 14.03 | 7.344e−11 | 620-638 | PR00011 | TYPE III EGF-LIKE |
| | | | | | | SIGNATURE |
| 1076 | PR00011D | 14.03 | 7.975e−16 | 711-729 | PR00011 | TYPE III EGF-LIKE |
| | | | | | | SIGNATURE |
| 1076 | PR00011D | 14.03 | 9.000e−15 | 2146-2164 | PR00011 | TYPE III EGF-LIKE |
| | | | | | | SIGNATURE |
| 1077 | BL01107A | 12.03 | 1.000e−40 | 10-52 | BL01107 | Ribosomal protein L27e |
| | | | | | | proteins. |
| 1077 | BL01107B | 16.28 | 1.000e−40 | 53-96 | BL01107 | Ribosomal protein L27e |
| | | | | | | proteins. |
| 1077 | BL01107C | 9.88 | 6.344e−26 | 122-143 | BL01107 | Ribosomal protein L27e |
| | | | | | | proteins. |
| 1077 | BL01170A | 12.34 | 9.124e−10 | 7-42 | BL01170 | Ribosomal protein L6e |
| | | | | | | proteins. |
| 1078 | BL01019A | 13.20 | 8.694e−18 | 107-146 | BL01019 | ADP-ribosylation |
| | | | | | | factors family |
| | | | | | | proteins. |
| 1078 | BL01019B | 19.49 | 8.603e−21 | 151-205 | BL01019 | ADP-ribosylation |
| | | | | | | factors family |
| | | | | | | proteins. |
| 1078 | BL01020C | 15.35 | 7.722e−20 | 135-185 | BL01020 | SAR1 family proteins. |
| 1078 | BL01115A | 10.22 | 9.151e−11 | 77-120 | BL01115 | GTP-binding nuclear |
| | | | | | | protein ran proteins. |
| 1078 | PR00328C | 13.16 | 2.111e−12 | 134-159 | PR00328 | GTP-BINDING SAR1 |
| | | | | | | PROTEIN SIGNATURE |
| 1078 | PR00328D | 12.56 | 8.071e−11 | 179-200 | PR00328 | GTP-BINDING SAR1 |
| | | | | | | PROTEIN SIGNATURE |
| 1080 | BL01243C | 14.19 | 5.111e−13 | 165-199 | BL01243 | Uncharacterized protein |
| | | | | | | family UPF0005 |
| | | | | | | proteins. |
| 1080 | BL01243D | 21.15 | 9.581e−11 | 247-281 | BL01243 | Uncharacterized protein |
| | | | | | | family UPF0005 |
| | | | | | | proteins. |
| 1081 | BL00292A | 22.87 | 6.786e−24 | 111-144 | BL00292 | Cyclins proteins. |
| 1081 | BL00292B | 20.31 | 3.739e−19 | 154-184 | BL00292 | Cyclins proteins. |
| 1082 | BL01013A | 25.14 | 1.000e−34 | 489-524 | BL01013 | Oxysterol-binding |
| | | | | | | protein family |
| | | | | | | proteins. |
| 1082 | BL01013B | 11.33 | 3.813e−14 | 554-564 | BL01013 | Oxysterol-binding |
| | | | | | | protein family |
| | | | | | | proteins. |
| 1082 | BL01013C | 9.97 | 6.211e−14 | 577-586 | BL01013 | Oxysterol-binding |
| | | | | | | protein family |
| | | | | | | proteins. |
| 1082 | BL01013D | 26.81 | 1.000e−37 | 775-818 | BL01013 | Oxysterol-binding |
| | | | | | | protein family |
| | | | | | | proteins. |
| 1083 | PF00075D | 10.71 | 8.929e−10 | 305-315 | PF00075 | RNase H. |
| 1085 | PR00237B | 13.50 | 6.000e−09 | 68-89 | PR00237 | RHODOPSIN-LIKE GPCR |
| | | | | | | SUPERFAMILY SIGNATURE |
| 1090 | BL00218B | 21.44 | 1.621e−10 | 103-134 | BL00218 | Amino acid permeases |
| | | | | | | proteins. |
| 1090 | BL00218D | 21.49 | 3.797e−11 | 271-315 | BL00218 | Amino acid permeases |
| | | | | | | proteins. |
| 1090 | BL00218E | 23.30 | 3.520e−10 | 352-391 | BL00218 | Amino acid permeases |
| | | | | | | proteins. |
| 1092 | BL00061A | 9.41 | 8.435e−09 | 107-117 | BL00061 | Short-chain |
| | | | | | | dehydrogenases/reductases |
| | | | | | | family proteins. |
| 1092 | BL00061B | 25.79 | 5.605e−09 | 171-208 | BL00061 | Short-chain |
| | | | | | | dehydrogenases/reductases |
| | | | | | | family proteins. |
| 1092 | BL00061C | 7.86 | 8.941e−09 | 213-222 | BL00061 | Short-chain |
| | | | | | | dehydrogenases/reductases |
| | | | | | | family proteins. |
| 1092 | PF00426W | 7.96 | 2.765e−09 | 25-51 | PF00426 | Outer Capsid protein |
| | | | | | | VP4 (Hemagglutinin). |
| 1092 | PR00080A | 9.32 | 6.226e−10 | 107-118 | PR00080 | ALCOHOL DEHYDROGENASE |
| | | | | | | SUPERFAMILY SIGNATURE |
| 1092 | PR00081A | 10.53 | 3.106e−10 | 31-48 | PR00081 | GLUCOSE/RIBITOL |
| | | | | | | DEHYDROGENASE FAMILY |
| | | | | | | SIGNATURE |
| 1092 | PR00081B | 10.38 | 6.727e−11 | 107-118 | PR00081 | GLUCOSE/RIBITOL |
| | | | | | | DEHYDROGENASE FAMILY |
| | | | | | | SIGNATURE |
| 1092 | PR00081E | 17.54 | 3.935e−10 | 212-229 | PR00081 | GLUCOSE/RIBITOL |
| | | | | | | DEHYDROGENASE FAMILY |
| | | | | | | SIGNATURE |
| 1093 | BL00115Z | 3.12 | 9.074e−09 | 157-205 | BL00115 | Eukaryotic RNA |
| | | | | | | polymerase II |
| | | | | | | heptapeptide repeat |
| | | | | | | proteins. |
| 1093 | PR00910A | 2.51 | 8.889e−10 | 203-215 | PR00910 | LUTEOVIRUS ORF6 PROTEIN |
| | | | | | | SIGNATURE |
| 1094 | BL00492A | 11.53 | 4.724e−18 | 16-32 | BL00492 | Clusterin proteins. |
| 1094 | BL00492B | 10.36 | 9.053e−28 | 40-62 | BL00492 | Clusterin proteins. |
| 1094 | BL00492C | 8.35 | 1.000e−38 | 66-99 | BL00492 | Clusterin proteins. |
| 1094 | BL00492D | 13.23 | 1.000e−40 | 100-136 | BL00492 | Clusterin proteins. |
| 1094 | BL00492E | 12.52 | 1.000e−40 | 142-190 | BL00492 | Clusterin proteins. |
| 1094 | BL00492F | 16.61 | 1.000e−40 | 232-273 | BL00492 | Clusterin proteins. |
| 1094 | BL00492G | 13.20 | 1.000e−40 | 299-348 | BL00492 | Clusterin proteins. |
| 1094 | BL00492H | 16.50 | 6.870e−40 | 427-463 | BL00492 | Clusterin proteins. |
| 1096 | BL00420A | 20.42 | 1.000e−08 | 248-276 | BL00420 | Speract receptor repeat |
| | | | | | | proteins domain |
| | | | | | | proteins. |
| 1096 | BL00420A | 20.42 | 1.000e−09 | 779-807 | BL00420 | Speract receptor repeat |
| | | | | | | proteins domain |
| | | | | | | proteins. |
| 1096 | BL00420A | 20.42 | 1.000e−10 | 1043-1071 | BL00420 | Speract receptor repeat |
| | | | | | | proteins domain |
| | | | | | | proteins. |
| 1096 | BL00420A | 20.42 | 1.138e−10 | 824-852 | BL00420 | Speract receptor repeat |
| | | | | | | proteins domain |
| | | | | | | proteins. |
| 1096 | BL00420A | 20.42 | 1.148e−10 | 377-405 | BL00420 | Speract receptor repeat |
| | | | | | | proteins domain |
| | | | | | | proteins. |
| 1096 | BL00420A | 20.42 | 1.164e−11 | 329-357 | BL00420 | Speract receptor repeat |
| | | | | | | proteins domain |
| | | | | | | proteins. |
| 1096 | BL00420A | 20.42 | 1.295e−10 | 608-636 | BL00420 | Speract receptor repeat |
| | | | | | | proteins domain |
| | | | | | | proteins. |
| 1096 | BL00420A | 20.42 | 1.491e−11 | 1145-1173 | BL00420 | Speract receptor repeat |
| | | | | | | proteins domain |
| | | | | | | proteins. |
| 1096 | BL00420A | 20.42 | 1.738e−10 | 887-915 | BL00420 | Speract receptor repeat |
| | | | | | | proteins domain |
| | | | | | | proteins. |
| 1096 | BL00420A | 20.42 | 1.831e−09 | 1040-1068 | BL00420 | Speract receptor repeat |
| | | | | | | proteins domain |
| | | | | | | proteins. |
| 1096 | BL00420A | 20.42 | 1.969e−09 | 530-558 | BL00420 | Speract receptor repeat |
| | | | | | | proteins domain |
| | | | | | | proteins. |
| 1096 | BL00420A | 20.42 | 2.108e−09 | 461-489 | BL00420 | Speract receptor repeat |
| | | | | | | proteins domain |
| | | | | | | proteins. |
| 1096 | BL00420A | 20.42 | 2.108e−09 | 842-870 | BL00420 | Speract receptor repeat |
| | | | | | | proteins domain |
| | | | | | | proteins. |
| 1096 | BL00420A | 20.42 | 2.108e−09 | 932-960 | BL00420 | Speract receptor repeat |
| | | | | | | proteins domain |
| | | | | | | proteins. |
| 1096 | BL00420A | 20.42 | 2.108e−09 | 956-984 | BL00420 | Speract receptor repeat |
| | | | | | | proteins domain |
| | | | | | | proteins. |
| 1096 | BL00420A | 20.42 | 2.246e−09 | 401-429 | BL00420 | Speract receptor repeat |
| | | | | | | proteins domain |
| | | | | | | proteins. |
| 1096 | BL00420A | 20.42 | 2.309e−11 | 347-375 | BL00420 | Speract receptor repeat |
| | | | | | | proteins domain |
| | | | | | | proteins. |
| 1096 | BL00420A | 20.42 | 2.309e−11 | 803-831 | BL00420 | Speract receptor repeat |
| | | | | | | proteins domain |
| | | | | | | proteins. |
| 1096 | BL00420A | 20.42 | 2.385e−09 | 833-861 | BL00420 | Speract receptor repeat |
| | | | | | | proteins domain |
| | | | | | | proteins. |
| 1096 | BL00420A | 20.42 | 2.473e−11 | 500-528 | BL00420 | Speract receptor repeat |
| | | | | | | proteins domain |
| | | | | | | proteins. |
| 1096 | BL00420A | 20.42 | 2.523e−09 | 1142-1170 | BL00420 | Speract receptor repeat |
| | | | | | | proteins domain |
| | | | | | | proteins. |
| 1096 | BL00420A | 20.42 | 2.662e−09 | 130-158 | BL00420 | Speract receptor repeat |
| | | | | | | proteins domain |
| | | | | | | proteins. |
| 1096 | BL00420A | 20.42 | 2.770e−10 | 674-702 | BL00420 | Speract receptor repeat |
| | | | | | | proteins domain |
| | | | | | | proteins. |
| 1096 | BL00420A | 20.42 | 3.077e−09 | 395-423 | BL00420 | Speract receptor repeat |
| | | | | | | proteins domain |
| | | | | | | proteins. |
| 1096 | BL00420A | 20.42 | 3.215e−09 | 133-161 | BL00420 | Speract receptor repeat |
| | | | | | | proteins domain |
| | | | | | | proteins. |
| 1096 | BL00420A | 20.42 | 3.215e−09 | 467-495 | BL00420 | Speract receptor repeat |
| | | | | | | proteins domain |
| | | | | | | proteins. |
| 1096 | BL00420A | 20.42 | 3.455e−11 | 551-579 | BL00420 | Speract receptor repeat |
| | | | | | | proteins domain |
| | | | | | | proteins. |
| 1096 | BL00420A | 20.42 | 3.618e−11 | 908-936 | BL00420 | Speract receptor repeat |
| | | | | | | proteins domain |
| | | | | | | proteins. |
| 1096 | BL00420A | 20.42 | 3.631e−09 | 1073-1101 | BL00420 | Speract receptor repeat |
| | | | | | | proteins domain |
| | | | | | | proteins. |
| 1096 | BL00420A | 20.42 | 3.631e−09 | 497-525 | BL00420 | Speract receptor repeat |
| | | | | | | proteins domain |
| | | | | | | proteins. |
| 1096 | BL00420A | 20.42 | 3.656e−10 | 539-567 | BL00420 | Speract receptor repeat |
| | | | | | | proteins domain |
| | | | | | | proteins. |
| 1096 | BL00420A | 20.42 | 3.803e−10 | 209-237 | BL00420 | Speract receptor repeat |
| | | | | | | proteins domain |
| | | | | | | proteins. |
| 1096 | BL00420A | 20.42 | 3.908e−09 | 515-543 | BL00420 | Speract receptor repeat |
| | | | | | | proteins domain |
| | | | | | | proteins. |
| 1096 | BL00420A | 20.42 | 3.908e−09 | 584-612 | BL00420 | Speract receptor repeat |
| | | | | | | proteins domain |
| | | | | | | proteins. |
| 1096 | BL00420A | 20.42 | 3.951e−10 | 881-909 | BL00420 | Speract receptor repeat |
| | | | | | | proteins domain |
| | | | | | | proteins. |
| 1096 | BL00420A | 20.42 | 4.046e−09 | 191-219 | BL00420 | Speract receptor repeat |
| | | | | | | proteins domain |
| | | | | | | proteins. |
| 1096 | BL00420A | 20.42 | 4.046e−09 | 341-369 | BL00420 | Speract receptor repeat |
| | | | | | | proteins domain |
| | | | | | | proteins. |
| 1096 | BL00420A | 20.42 | 4.246e−10 | 1013-1041 | BL00420 | Speract receptor repeat |
| | | | | | | proteins domain |
| | | | | | | proteins. |
| 1096 | BL00420A | 20.42 | 4.273e−11 | 299-327 | BL00420 | Speract receptor repeat |
| | | | | | | proteins domain |
| | | | | | | proteins. |
| 1096 | BL00420A | 20.42 | 4.323e−09 | 1046-1074 | BL00420 | Speract receptor repeat |
| | | | | | | proteins domain |
| | | | | | | proteins. |
| 1096 | BL00420A | 20.42 | 4.323e−09 | 242-270 | BL00420 | Speract receptor repeat |
| | | | | | | proteins domain |
| | | | | | | proteins. |
| 1096 | BL00420A | 20.42 | 4.490e−13 | 566-594 | BL00420 | Speract receptor repeat |
| | | | | | | proteins domain |
| | | | | | | proteins. |
| 1096 | BL00420A | 20.42 | 4.738e−09 | 440-468 | BL00420 | Speract receptor repeat |
| | | | | | | proteins domain |
| | | | | | | proteins. |
| 1096 | BL00420A | 20.42 | 4.738e−09 | 641-669 | BL00420 | Speract receptor repeat |
| | | | | | | proteins domain |
| | | | | | | proteins. |
| 1096 | BL00420A | 20.42 | 4.927e−11 | 1103-1131 | BL00420 | Speract receptor repeat |
| | | | | | | proteins domain |
| | | | | | | proteins. |
| 1096 | BL00420A | 20.42 | 4.984e−10 | 707-735 | BL00420 | Speract receptor repeat |
| | | | | | | proteins domain |
| | | | | | | proteins. |
| 1096 | BL00420A | 20.42 | 4.984e−10 | 878-906 | BL00420 | Speract receptor repeat |
| | | | | | | proteins domain |
| | | | | | | proteins. |
| 1096 | BL00420A | 20.42 | 5.154e−09 | 1166-1194 | BL00420 | Speract receptor repeat |
| | | | | | | proteins domain |
| | | | | | | proteins. |
| 1096 | BL00420A | 20.42 | 5.154e−09 | 443-471 | BL00420 | Speract receptor repeat |
| | | | | | | proteins domain |
| | | | | | | proteins. |
| 1096 | BL00420A | 20.42 | 5.154e−09 | 647-675 | BL00420 | Speract receptor repeat |
| | | | | | | proteins domain |
| | | | | | | proteins. |
| 1096 | BL00420A | 20.42 | 5.292e−09 | 710-738 | BL00420 | Speract receptor repeat |
| | | | | | | proteins domain |
| | | | | | | proteins. |
| 1096 | BL00420A | 20.42 | 5.292e−09 | 935-963 | BL00420 | Speract receptor repeat |
| | | | | | | proteins domain |
| | | | | | | proteins. |
| 1096 | BL00420A | 20.42 | 5.426e−10 | 869-897 | BL00420 | Speract receptor repeat |
| | | | | | | proteins domain |
| | | | | | | proteins. |
| 1096 | BL00420A | 20.42 | 5.569e−09 | 127-155 | BL00420 | Speract receptor repeat |
| | | | | | | proteins domain |
| | | | | | | proteins. |
| 1096 | BL00420A | 20.42 | 5.869e−10 | 872-900 | BL00420 | Speract receptor repeat |
| | | | | | | proteins domain |
| | | | | | | proteins. |
| 1096 | BL00420A | 20.42 | 5.909e−11 | 968-996 | BL00420 | Speract receptor repeat |
| | | | | | | proteins domain |
| | | | | | | proteins. |
| 1096 | BL00420A | 20.42 | 6.123e−09 | 1124-1152 | BL00420 | Speract receptor repeat |
| | | | | | | proteins domain |
| | | | | | | proteins. |
| 1096 | BL00420A | 20.42 | 6.123e−09 | 488-516 | BL00420 | Speract receptor repeat |
| | | | | | | proteins domain |
| | | | | | | proteins. |
| 1096 | BL00420A | 20.42 | 6.123e−09 | 590-618 | BL00420 | Speract receptor repeat |
| | | | | | | proteins domain |
| | | | | | | proteins. |
| 1096 | BL00420A | 20.42 | 6.143e−13 | 653-681 | BL00420 | Speract receptor repeat |
| | | | | | | proteins domain |
| | | | | | | proteins. |
| 1096 | BL00420A | 20.42 | 6.262e−09 | 791-819 | BL00420 | Speract receptor repeat |
| | | | | | | proteins domain |
| | | | | | | proteins. |
| 1096 | BL00420A | 20.42 | 6.400e−09 | 371-399 | BL00420 | Speract receptor repeat |
| | | | | | | proteins domain |
| | | | | | | proteins. |
| 1096 | BL00420A | 20.42 | 6.400e−11 | 572-600 | BL00420 | Speract receptor repeat |
| | | | | | | proteins domain |
| | | | | | | proteins. |
| 1096 | BL00420A | 20.42 | 6.459e−10 | 263-291 | BL00420 | Speract receptor repeat |
| | | | | | | proteins domain |
| | | | | | | proteins. |
| 1096 | BL00420A | 20.42 | 6.538e−09 | 359-387 | BL00420 | Speract receptor repeat |
| | | | | | | proteins domain |
| | | | | | | proteins. |
| 1096 | BL00420A | 20.42 | 6.815e−09 | 521-549 | BL00420 | Speract receptor repeat |
| | | | | | | proteins domain |
| | | | | | | proteins. |
| 1096 | BL00420A | 20.42 | 7.092e−09 | 1160-1188 | BL00420 | Speract receptor repeat |
| | | | | | | proteins domain |
| | | | | | | proteins. |
| 1096 | BL00420A | 20.42 | 7.092e−09 | 731-759 | BL00420 | Speract receptor repeat |
| | | | | | | proteins domain |
| | | | | | | proteins. |
| 1096 | BL00420A | 20.42 | 7.092e−09 | 890-918 | BL00420 | Speract receptor repeat |
| | | | | | | proteins domain |
| | | | | | | proteins. |
| 1096 | BL00420A | 20.42 | 7.197e−10 | 317-345 | BL00420 | Speract receptor repeat |
| | | | | | | proteins domain |
| | | | | | | proteins. |
| 1096 | BL00420A | 20.42 | 7.197e−10 | 425-453 | BL00420 | Speract receptor repeat |
| | | | | | | proteins domain |
| | | | | | | proteins. |
| 1096 | BL00420A | 20.42 | 7.231e−09 | 836-864 | BL00420 | Speract receptor repeat |
| | | | | | | proteins domain |
| | | | | | | proteins. |
| 1096 | BL00420A | 20.42 | 7.369e−09 | 218-246 | BL00420 | Speract receptor repeat |
| | | | | | | proteins domain |
| | | | | | | proteins. |
| 1096 | BL00420A | 20.42 | 7.508e−09 | 1148-1176 | BL00420 | Speract receptor repeat |
| | | | | | | proteins domain |
| | | | | | | proteins. |
| 1096 | BL00420A | 20.42 | 7.508e−09 | 992-1020 | BL00420 | Speract receptor repeat |
| | | | | | | proteins domain |
| | | | | | | proteins. |
| 1096 | BL00420A | 20.42 | 7.646e−09 | 227-255 | BL00420 | Speract receptor repeat |
| | | | | | | proteins domain |
| | | | | | | proteins. |
| 1096 | BL00420A | 20.42 | 7.646e−09 | 491-519 | BL00420 | Speract receptor repeat |
| | | | | | | proteins domain |
| | | | | | | proteins. |
| 1096 | BL00420A | 20.42 | 7.787e−10 | 185-213 | BL00420 | Speract receptor repeat |
| | | | | | | proteins domain |
| | | | | | | proteins. |
| 1096 | BL00420A | 20.42 | 8.036e−11 | 722-750 | BL00420 | Speract receptor repeat |
| | | | | | | proteins domain |
| | | | | | | proteins. |
| 1096 | BL00420A | 20.42 | 8.071e−15 | 410-438 | BL00420 | Speract receptor repeat |
| | | | | | | proteins domain |
| | | | | | | proteins. |
| 1096 | BL00420A | 20.42 | 8.082e−10 | 206-234 | BL00420 | Speract receptor repeat |
| | | | | | | proteins domain |
| | | | | | | proteins. |
| 1096 | BL00420A | 20.42 | 8.230e−10 | 518-546 | BL00420 | Speract receptor repeat |
| | | | | | | proteins domain |
| | | | | | | proteins. |
| 1096 | BL00420A | 20.42 | 8.338e−09 | 812-840 | BL00420 | Speract receptor repeat |
| | | | | | | proteins domain |
| | | | | | | proteins. |
| 1096 | BL00420A | 20.42 | 8.347e−13 | 944-972 | BL00420 | Speract receptor repeat |
| | | | | | | proteins domain |
| | | | | | | proteins. |
| 1096 | BL00420A | 20.42 | 8.377e−10 | 284-312 | BL00420 | Speract receptor repeat |
| | | | | | | proteins domain |
| | | | | | | proteins. |
| 1096 | BL00420A | 20.42 | 8.377e−10 | 587-615 | BL00420 | Speract receptor repeat |
| | | | | | | proteins domain |
| | | | | | | proteins. |
| 1096 | BL00420A | 20.42 | 8.477e−09 | 542-570 | BL00420 | Speract receptor repeat |
| | | | | | | proteins domain |
| | | | | | | proteins. |
| 1096 | BL00420A | 20.42 | 8.477e−09 | 635-663 | BL00420 | Speract receptor repeat |
| | | | | | | proteins domain |
| | | | | | | proteins. |
| 1096 | BL00420A | 20.42 | 8.525e−10 | 773-801 | BL00420 | Speract receptor repeat |
| | | | | | | proteins domain |
| | | | | | | proteins. |
| 1096 | BL00420A | 20.42 | 8.531e−13 | 485-513 | BL00420 | Speract receptor repeat |
| | | | | | | proteins domain |
| | | | | | | proteins. |
| 1096 | BL00420A | 20.42 | 8.615e−12 | 605-633 | BL00420 | Speract receptor repeat |
| | | | | | | proteins domain |
| | | | | | | proteins. |
| 1096 | BL00420A | 20.42 | 8.754e−09 | 758-786 | BL00420 | Speract receptor repeat |
| | | | | | | proteins domain |
| | | | | | | proteins. |
| 1096 | BL00420A | 20.42 | 8.892e−09 | 230-258 | BL00420 | Speract receptor repeat |
| | | | | | | proteins domain |
| | | | | | | proteins. |
| 1096 | BL00420A | 20.42 | 8.892e−09 | 506-534 | BL00420 | Speract receptor repeat |
| | | | | | | proteins domain |
| | | | | | | proteins. |
| 1096 | BL00420A | 20.42 | 8.892e−09 | 911-939 | BL00420 | Speract receptor repeat |
| | | | | | | proteins domain |
| | | | | | | proteins. |
| 1096 | BL00420A | 20.42 | 9.031e−09 | 398-426 | BL00420 | Speract receptor repeat |
| | | | | | | proteins domain |
| | | | | | | proteins. |
| 1096 | BL00420A | 20.42 | 9.031e−09 | 677-705 | BL00420 | Speract receptor repeat |
| | | | | | | proteins domain |
| | | | | | | proteins. |
| 1096 | BL00420A | 20.42 | 9.031e−09 | 860-888 | BL00420 | Speract receptor repeat |
| | | | | | | proteins domain |
| | | | | | | proteins. |
| 1096 | BL00420A | 20.42 | 9.115e−10 | 947-975 | BL00420 | Speract receptor repeat |
| | | | | | | proteins domain |
| | | | | | | proteins. |
| 1096 | BL00420A | 20.42 | 9.135e−12 | 800-828 | BL00420 | Speract receptor repeat |
| | | | | | | proteins domain |
| | | | | | | proteins. |
| 1096 | BL00420A | 20.42 | 9.169e−09 | 1121-1149 | BL00420 | Speract receptor repeat |
| | | | | | | proteins domain |
| | | | | | | proteins. |
| 1096 | BL00420A | 20.42 | 9.182e−11 | 629-657 | BL00420 | Speract receptor repeat |
| | | | | | | proteins domain |
| | | | | | | proteins. |
| 1096 | BL00420A | 20.42 | 9.262e−10 | 136-164 | BL00420 | Speract receptor repeat |
| | | | | | | proteins domain |
| | | | | | | proteins. |
| 1096 | BL00420A | 20.42 | 9.345e−11 | 197-225 | BL00420 | Speract receptor repeat |
| | | | | | | proteins domain |
| | | | | | | proteins. |
| 1096 | BL00420A | 20.42 | 9.585e−09 | 407-435 | BL00420 | Speract receptor repeat |
| | | | | | | proteins domain |
| | | | | | | proteins. |
| 1096 | BL00420A | 20.42 | 9.585e−09 | 971-999 | BL00420 | Speract receptor repeat |
| | | | | | | proteins domain |
| | | | | | | proteins. |
| 1096 | BL00420A | 20.42 | 9.852e−10 | 719-747 | BL00420 | Speract receptor repeat |
| | | | | | | proteins domain |
| | | | | | | proteins. |
| 1096 | BL01113A | 17.99 | 1.000e−08 | 1022-1048 | BL01113 | C1q domain proteins. |
| 1096 | BL01113A | 17.99 | 1.000e−09 | 593-619 | BL01113 | C1q domain proteins. |
| 1096 | BL01113A | 17.99 | 1.173e−09 | 1145-1171 | BL01113 | C1q domain proteins. |
| 1096 | BL01113A | 17.99 | 1.346e−09 | 461-487 | BL01113 | C1q domain proteins. |
| 1096 | BL01113A | 17.99 | 1.519e−09 | 524-550 | BL01113 | C1q domain proteins. |
| 1096 | BL01113A | 17.99 | 1.614e−11 | 139-165 | BL01113 | C1q domain proteins. |
| 1096 | BL01113A | 17.99 | 1.614e−11 | 584-610 | BL01113 | C1q domain proteins. |
| 1096 | BL01113A | 17.99 | 1.692e−09 | 209-235 | BL01113 | C1q domain proteins. |
| 1096 | BL01113A | 17.99 | 1.692e−09 | 410-436 | BL01113 | C1q domain proteins. |
| 1096 | BL01113A | 17.99 | 1.692e−09 | 644-670 | BL01113 | C1q domain proteins. |
| 1096 | BL01113A | 17.99 | 1.818e−11 | 896-922 | BL01113 | C1q domain proteins. |
| 1096 | BL01113A | 17.99 | 1.957e−10 | 815-841 | BL01113 | C1q domain proteins. |
| 1096 | BL01113A | 17.99 | 2.038e−09 | 233-259 | BL01113 | C1q domain proteins. |
| 1096 | BL01113A | 17.99 | 2.154e−12 | 515-541 | BL01113 | C1q domain proteins. |
| 1096 | BL01113A | 17.99 | 2.227e−11 | 191-217 | BL01113 | C1q domain proteins. |
| 1096 | BL01113A | 17.99 | 2.241e−16 | 130-156 | BL01113 | C1q domain proteins. |
| 1096 | BL01113A | 17.99 | 2.532e−10 | 419-445 | BL01113 | C1q domain proteins. |
| 1096 | BL01113A | 17.99 | 2.558e−09 | 566-592 | BL01113 | C1q domain proteins. |
| 1096 | BL01113A | 17.99 | 2.731e−09 | 1040-1066 | BL01113 | C1q domain proteins. |
| 1096 | BL01113A | 17.99 | 2.731e−09 | 365-391 | BL01113 | C1q domain proteins. |
| 1096 | BL01113A | 17.99 | 2.846e−12 | 716-742 | BL01113 | C1q domain proteins. |
| 1096 | BL01113A | 17.99 | 2.846e−12 | 806-832 | BL01113 | C1q domain proteins. |
| 1096 | BL01113A | 17.99 | 2.904e−09 | 521-547 | BL01113 | C1q domain proteins. |
| 1096 | BL01113A | 17.99 | 2.915e−10 | 965-991 | BL01113 | C1q domain proteins. |
| 1096 | BL01113A | 17.99 | 3.045e−11 | 218-244 | BL01113 | C1q domain proteins. |
| 1096 | BL01113A | 17.99 | 3.077e−09 | 995-1021 | BL01113 | C1q domain proteins. |
| 1096 | BL01113A | 17.99 | 3.106e−10 | 491-517 | BL01113 | C1q domain proteins. |
| 1096 | BL01113A | 17.99 | 3.250e−09 | 338-364 | BL01113 | C1q domain proteins. |
| 1096 | BL01113A | 17.99 | 3.250e−11 | 482-508 | BL01113 | C1q domain proteins. |
| 1096 | BL01113A | 17.99 | 3.298e−10 | 473-499 | BL01113 | C1q domain proteins. |
| 1096 | BL01113A | 17.99 | 3.298e−10 | 560-586 | BL01113 | C1q domain proteins. |
| 1096 | BL01113A | 17.99 | 3.298e−10 | 929-955 | BL01113 | C1q domain proteins. |
| 1096 | BL01113A | 17.99 | 3.489e−10 | 284-310 | BL01113 | C1q domain proteins. |
| 1096 | BL01113A | 17.99 | 3.489e−10 | 377-403 | BL01113 | C1q domain proteins. |
| 1096 | BL01113A | 17.99 | 3.489e−10 | 638-664 | BL01113 | C1q domain proteins. |
| 1096 | BL01113A | 17.99 | 3.596e−09 | 989-1015 | BL01113 | C1q domain proteins. |
| 1096 | BL01113A | 17.99 | 3.659e−11 | 911-937 | BL01113 | C1q domain proteins. |
| 1096 | BL01113A | 17.99 | 3.681e−10 | 242-268 | BL01113 | C1q domain proteins. |
| 1096 | BL01113A | 17.99 | 3.769e−09 | 124-150 | BL01113 | C1q domain proteins. |
| 1096 | BL01113A | 17.99 | 3.769e−09 | 467-493 | BL01113 | C1q domain proteins. |
| 1096 | BL01113A | 17.99 | 3.769e−09 | 917-943 | BL01113 | C1q domain proteins. |
| 1096 | BL01113A | 17.99 | 3.769e−12 | 197-223 | BL01113 | C1q domain proteins. |
| 1096 | BL01113A | 17.99 | 4.064e−10 | 773-799 | BL01113 | C1q domain proteins. |
| 1096 | BL01113A | 17.99 | 4.068e−11 | 569-595 | BL01113 | C1q domain proteins. |
| 1096 | BL01113A | 17.99 | 4.115e−09 | 878-904 | BL01113 | C1q domain proteins. |
| 1096 | BL01113A | 17.99 | 4.162e−13 | 548-574 | BL01113 | C1q domain proteins. |
| 1096 | BL01113A | 17.99 | 4.288e−09 | 1031-1057 | BL01113 | C1q domain proteins. |
| 1096 | BL01113A | 17.99 | 4.447e−10 | 653-679 | BL01113 | C1q domain proteins. |
| 1096 | BL01113A | 17.99 | 4.447e−10 | 710-736 | BL01113 | C1q domain proteins. |
| 1096 | BL01113A | 17.99 | 4.462e−09 | 662-688 | BL01113 | C1q domain proteins. |
| 1096 | BL01113A | 17.99 | 4.477e−11 | 926-952 | BL01113 | C1q domain proteins. |
| 1096 | BL01113A | 17.99 | 4.635e−09 | 422-448 | BL01113 | C1q domain proteins. |
| 1096 | BL01113A | 17.99 | 4.638e−10 | 452-478 | BL01113 | C1q domain proteins. |
| 1096 | BL01113A | 17.99 | 4.682e−11 | 133-159 | BL01113 | C1q domain proteins. |
| 1096 | BL01113A | 17.99 | 4.682e−11 | 920-946 | BL01113 | C1q domain proteins. |
| 1096 | BL01113A | 17.99 | 4.692e−12 | 809-835 | BL01113 | C1q domain proteins. |
| 1096 | BL01113A | 17.99 | 4.774e−15 | 428-454 | BL01113 | C1q domain proteins. |
| 1096 | BL01113A | 17.99 | 4.808e−09 | 374-400 | BL01113 | C1q domain proteins. |
| 1096 | BL01113A | 17.99 | 4.808e−09 | 890-916 | BL01113 | C1q domain proteins. |
| 1096 | BL01113A | 17.99 | 4.808e−17 | 136-162 | BL01113 | C1q domain proteins. |
| 1096 | BL01113A | 17.99 | 4.892e−13 | 887-913 | BL01113 | C1q domain proteins. |
| 1096 | BL01113A | 17.99 | 5.154e−12 | 404-430 | BL01113 | C1q domain proteins. |
| 1096 | BL01113A | 17.99 | 5.295e−11 | 1181-1207 | BL01113 | C1q domain proteins. |
| 1096 | BL01113A | 17.99 | 5.327e−09 | 497-523 | BL01113 | C1q domain proteins. |
| 1096 | BL01113A | 17.99 | 5.327e−09 | 674-700 | BL01113 | C1q domain proteins. |
| 1096 | BL01113A | 17.99 | 5.500e−09 | 821-847 | BL01113 | C1q domain proteins. |
| 1096 | BL01113A | 17.99 | 5.500e−09 | 944-970 | BL01113 | C1q domain proteins. |
| 1096 | BL01113A | 17.99 | 5.500e−11 | 1166-1192 | BL01113 | C1q domain proteins. |
| 1096 | BL01113A | 17.99 | 5.596e−10 | 386-412 | BL01113 | C1q domain proteins. |
| 1096 | BL01113A | 17.99 | 5.787e−10 | 335-361 | BL01113 | C1q domain proteins. |
| 1096 | BL01113A | 17.99 | 6.019e−09 | 263-289 | BL01113 | C1q domain proteins. |
| 1096 | BL01113A | 17.99 | 6.019e−09 | 914-940 | BL01113 | C1q domain proteins. |
| 1096 | BL01113A | 17.99 | 6.077e−12 | 188-214 | BL01113 | C1q domain proteins. |
| 1096 | BL01113A | 17.99 | 6.077e−12 | 290-316 | BL01113 | C1q domain proteins. |
| 1096 | BL01113A | 17.99 | 6.114e−11 | 224-250 | BL01113 | C1q domain proteins. |
| 1096 | BL01113A | 17.99 | 6.170e−10 | 791-817 | BL01113 | C1q domain proteins. |
| 1096 | BL01113A | 17.99 | 6.192e−09 | 380-406 | BL01113 | C1q domain proteins. |
| 1096 | BL01113A | 17.99 | 6.308e−12 | 557-583 | BL01113 | C1q domain proteins. |
| 1096 | BL01113A | 17.99 | 6.308e−12 | 731-757 | BL01113 | C1q domain proteins. |
| 1096 | BL01113A | 17.99 | 6.362e−10 | 221-247 | BL01113 | C1q domain proteins. |
| 1096 | BL01113A | 17.99 | 6.362e−10 | 872-898 | BL01113 | C1q domain proteins. |
| 1096 | BL01113A | 17.99 | 6.455e−14 | 842-868 | BL01113 | C1q domain proteins. |
| 1096 | BL01113A | 17.99 | 6.538e−09 | 293-319 | BL01113 | C1q domain proteins. |
| 1096 | BL01113A | 17.99 | 6.553e−10 | 1142-1168 | BL01113 | C1q domain proteins. |
| 1096 | BL01113A | 17.99 | 6.745e−10 | 575-601 | BL01113 | C1q domain proteins. |
| 1096 | BL01113A | 17.99 | 6.769e−12 | 1046-1072 | BL01113 | C1q domain proteins. |
| 1096 | BL01113A | 17.99 | 6.936e−10 | 947-973 | BL01113 | C1q domain proteins. |
| 1096 | BL01113A | 17.99 | 7.058e−09 | 608-634 | BL01113 | C1q domain proteins. |
| 1096 | BL01113A | 17.99 | 7.128e−10 | 248-274 | BL01113 | C1q domain proteins. |
| 1096 | BL01113A | 17.99 | 7.128e−10 | 938-964 | BL01113 | C1q domain proteins. |
| 1096 | BL01113A | 17.99 | 7.231e−12 | 121-147 | BL01113 | C1q domain proteins. |
| 1096 | BL01113A | 17.99 | 7.319e−10 | 182-208 | BL01113 | C1q domain proteins. |
| 1096 | BL01113A | 17.99 | 7.319e−10 | 563-589 | BL01113 | C1q domain proteins. |
| 1096 | BL01113A | 17.99 | 7.341e−11 | 974-1000 | BL01113 | C1q domain proteins. |
| 1096 | BL01113A | 17.99 | 7.404e−09 | 539-565 | BL01113 | C1q domain proteins. |
| 1096 | BL01113A | 17.99 | 7.511e−10 | 1001-1027 | BL01113 | C1q domain proteins. |
| 1096 | BL01113A | 17.99 | 7.511e−10 | 206-232 | BL01113 | C1q domain proteins. |
| 1096 | BL01113A | 17.99 | 7.511e−10 | 851-877 | BL01113 | C1q domain proteins. |
| 1096 | BL01113A | 17.99 | 7.545e−14 | 239-265 | BL01113 | C1q domain proteins. |
| 1096 | BL01113A | 17.99 | 7.545e−14 | 299-325 | BL01113 | C1q domain proteins. |
| 1096 | BL01113A | 17.99 | 7.577e−09 | 1175-1201 | BL01113 | C1q domain proteins. |
| 1096 | BL01113A | 17.99 | 7.702e−10 | 656-682 | BL01113 | C1q domain proteins. |
| 1096 | BL01113A | 17.99 | 7.750e−09 | 1127-1153 | BL01113 | C1q domain proteins. |
| 1096 | BL01113A | 17.99 | 7.750e−09 | 203-229 | BL01113 | C1q domain proteins. |
| 1096 | BL01113A | 17.99 | 7.750e−09 | 332-358 | BL01113 | C1q domain proteins. |
| 1096 | BL01113A | 17.99 | 7.923e−09 | 485-511 | BL01113 | C1q domain proteins. |
| 1096 | BL01113A | 17.99 | 7.955e−11 | 1010-1036 | BL01113 | C1q domain proteins. |
| 1096 | BL01113A | 17.99 | 8.085e−10 | 551-577 | BL01113 | C1q domain proteins. |
| 1096 | BL01113A | 17.99 | 8.096e−09 | 998-1024 | BL01113 | C1q domain proteins. |
| 1096 | BL01113A | 17.99 | 8.159e−11 | 1151-1177 | BL01113 | C1q domain proteins. |
| 1096 | BL01113A | 17.99 | 8.277e−10 | 506-532 | BL01113 | C1q domain proteins. |
| 1096 | BL01113A | 17.99 | 8.385e−12 | 1118-1144 | BL01113 | C1q domain proteins. |
| 1096 | BL01113A | 17.99 | 8.442e−09 | 145-171 | BL01113 | C1q domain proteins. |
| 1096 | BL01113A | 17.99 | 8.442e−09 | 254-280 | BL01113 | C1q domain proteins. |
| 1096 | BL01113A | 17.99 | 8.442e−09 | 941-967 | BL01113 | C1q domain proteins. |
| 1096 | BL01113A | 17.99 | 8.468e−10 | 1160-1186 | BL01113 | C1q domain proteins. |
| 1096 | BL01113A | 17.99 | 8.568e−11 | 695-721 | BL01113 | C1q domain proteins. |
| 1096 | BL01113A | 17.99 | 8.615e−09 | 215-241 | BL01113 | C1q domain proteins. |
| 1096 | BL01113A | 17.99 | 8.615e−09 | 650-676 | BL01113 | C1q domain proteins. |
| 1096 | BL01113A | 17.99 | 8.615e−12 | 647-673 | BL01113 | C1q domain proteins. |
| 1096 | BL01113A | 17.99 | 8.660e−10 | 683-709 | BL01113 | C1q domain proteins. |
| 1096 | BL01113A | 17.99 | 8.660e−10 | 758-784 | BL01113 | C1q domain proteins. |
| 1096 | BL01113A | 17.99 | 8.773e−11 | 1121-1147 | BL01113 | C1q domain proteins. |
| 1096 | BL01113A | 17.99 | 8.788e−09 | 281-307 | BL01113 | C1q domain proteins. |
| 1096 | BL01113A | 17.99 | 8.851e−10 | 413-439 | BL01113 | C1q domain proteins. |
| 1096 | BL01113A | 17.99 | 8.962e−09 | 641-667 | BL01113 | C1q domain proteins. |
| 1096 | BL01113A | 17.99 | 8.962e−09 | 818-844 | BL01113 | C1q domain proteins. |
| 1096 | BL01113A | 17.99 | 8.962e−09 | 956-982 | BL01113 | C1q domain proteins. |
| 1096 | BL01113A | 17.99 | 9.043e−10 | 1157-1183 | BL01113 | C1q domain proteins. |
| 1096 | BL01113A | 17.99 | 9.043e−10 | 308-334 | BL01113 | C1q domain proteins. |
| 1096 | BL01113A | 17.99 | 9.043e−10 | 623-649 | BL01113 | C1q domain proteins. |
| 1096 | BL01113A | 17.99 | 9.077e−12 | 185-211 | BL01113 | C1q domain proteins. |
| 1096 | BL01113A | 17.99 | 9.135e−09 | 389-415 | BL01113 | C1q domain proteins. |
| 1096 | BL01113A | 17.99 | 9.135e−09 | 488-514 | BL01113 | C1q domain proteins. |
| 1096 | BL01113A | 17.99 | 9.182e−11 | 740-766 | BL01113 | C1q domain proteins. |
| 1096 | BL01113A | 17.99 | 9.308e−09 | 443-469 | BL01113 | C1q domain proteins. |
| 1096 | BL01113A | 17.99 | 9.426e−10 | 1025-1051 | BL01113 | C1q domain proteins. |
| 1096 | BL01113A | 17.99 | 9.426e−10 | 536-562 | BL01113 | C1q domain proteins. |
| 1096 | BL01113A | 17.99 | 9.455e−14 | 800-826 | BL01113 | C1q domain proteins. |
| 1096 | BL01113A | 17.99 | 9.481e−09 | 704-730 | BL01113 | C1q domain proteins. |
| 1096 | BL01113A | 17.99 | 9.514e−13 | 1043-1069 | BL01113 | C1q domain proteins. |
| 1096 | BL01113A | 17.99 | 9.591e−11 | 476-502 | BL01113 | C1q domain proteins. |
| 1096 | BL01113A | 17.99 | 9.617e−10 | 194-220 | BL01113 | C1q domain proteins. |
| 1096 | BL01113A | 17.99 | 9.617e−10 | 395-421 | BL01113 | C1q domain proteins. |
| 1096 | BL01113A | 17.99 | 9.617e−10 | 770-796 | BL01113 | C1q domain proteins. |
| 1096 | BL01113A | 17.99 | 9.654e−09 | 200-226 | BL01113 | C1q domain proteins. |
| 1096 | BL01113A | 17.99 | 9.654e−09 | 230-256 | BL01113 | C1q domain proteins. |
| 1096 | BL01113A | 17.99 | 9.757e−13 | 127-153 | BL01113 | C1q domain proteins. |
| 1096 | BL01113A | 17.99 | 9.757e−13 | 142-168 | BL01113 | C1q domain proteins. |
| 1096 | BL01113A | 17.99 | 9.809e−10 | 722-748 | BL01113 | C1q domain proteins. |
| 1096 | BL01113A | 17.99 | 9.827e−09 | 545-571 | BL01113 | C1q domain proteins. |
| 1096 | BL01113A | 17.99 | 9.827e−09 | 692-718 | BL01113 | C1q domain proteins. |
| 1096 | BL01208B | 15.83 | 5.950e−13 | 84-98 | BL01208 | VWFC domain proteins. |
| 1096 | DM01418A | 20.83 | 1.000e−40 | 1255-1302 | DM01418 | 352 FIBRILLAR COLLAGEN |
| | | | | | | CARBOXYL-TERMINAL. |
| 1096 | DM01418B | 22.51 | 1.000e−40 | 1354-1395 | DM01418 | 352 FIBRILLAR COLLAGEN |
| | | | | | | CARBOXYL-TERMINAL. |
| 1096 | DM01418C | 20.48 | 5.500e−40 | 1425-1466 | DM01418 | 352 FIBRILLAR COLLAGEN |
| | | | | | | CARBOXYL-TERMINAL. |
| 1096 | PR00049D | 0.00 | 5.271e−09 | 1181-1195 | PR00049 | WILM'S TUMOUR PROTEIN |
| | | | | | | SIGNATURE |
| 1096 | PR00049D | 0.00 | 5.424e−09 | 1180-1194 | PR00049 | WILM'S TUMOUR PROTEIN |
| | | | | | | SIGNATURE |
| 1096 | PR00049D | 0.00 | 6.186e−09 | 142-156 | PR00049 | WILM'S TUMOUR PROTEIN |
| | | | | | | SIGNATURE |
| 1096 | PR00049D | 0.00 | 8.571e−10 | 1177-1191 | PR00049 | WILM'S TUMOUR PROTEIN |
| | | | | | | SIGNATURE |
| 1100 | BL01019A | 13.20 | 4.156e−12 | 234-273 | BL01019 | ADP-ribosylation |
| | | | | | | factors family |
| | | | | | | proteins. |
| 1100 | PR00318A | 7.84 | 1.600e−19 | 88-103 | PR00318 | ALPHA G-PROTEIN |
| | | | | | | (TRANSDUCIN) SIGNATURE |
| 1100 | PR00318B | 14.79 | 9.000e−28 | 221-243 | PR00318 | ALPHA G-PROTEIN |
| | | | | | | (TRANSDUCIN) SIGNATURE |
| 1100 | PR000318C | 12.09 | 3.571e−24 | 250-267 | PR00318 | ALPHA G-PROTEIN |
| | | | | | | (TRANSDUCIN) SIGNATURE |
| 1100 | PR00318D | 16.28 | 1.563e−30 | 272-300 | PR00318 | ALPHA G-PROTEIN |
| | | | | | | (TRANSDUCIN) SIGNATURE |
| 1100 | PR00318E | 7.23 | 2.125e−13 | 318-327 | PR00318 | ALPHA G-PROTEIN |
| | | | | | | (TRANSDUCIN) SIGNATURE |
| 1100 | PR00440A | 9.18 | 5.645e−15 | 88-103 | PR00440 | G-PROTEIN ALPHA SUBUNIT |
| | | | | | | GROUP 12 SIGNATURE |
| 1100 | PR00440B | 13.27 | 6.260e−13 | 221-243 | PR00440 | G-PROTEIN ALPHA SUBUNIT |
| | | | | | | GROUP 12 SIGNATURE |
| 1100 | PR00440C | 9.54 | 6.885e−18 | 250-267 | PR00440 | G-PROTEIN ALPHA SUBUNIT |
| | | | | | | GROUP 12 SIGNATURE |
| 1100 | PR00440D | 8.15 | 1.303e−15 | 272-300 | PR00440 | G-PROTEIN ALPHA SUBUNIT |
| | | | | | | GROUP 12 SIGNATURE |
| 1100 | PR00440E | 11.16 | 3.880e−10 | 319-327 | PR00440 | G-PROTEIN ALPHA SUBUNIT |
| | | | | | | GROUP 12 SIGNATURE |
| 1100 | PR00441A | 10.69 | 1.375e−19 | 88-103 | PR00441 | G-PROTEIN ALPHA SUBUNIT |
| | | | | | | GROUP I SIGNATURE |
| 1100 | PR00441B | 16.16 | 4.000e−29 | 221-243 | PR00441 | G-PROTEIN ALPHA SUBUNIT |
| | | | | | | GROUP I SIGNATURE |
| 1100 | PR00441C | 14.17 | 1.409e−24 | 250-267 | PR00441 | G-PROTEIN ALPHA SUBUNIT |
| | | | | | | GROUP I SIGNATURE |
| 1100 | PR00441D | 14.44 | 9.250e−36 | 272-300 | PR00441 | G-PROTEIN ALPHA SUBUNIT |
| | | | | | | GROUP I SIGNATURE |
| 1100 | PR00441E | 8.35 | 1.214e−11 | 319-327 | PR00441 | G-PROTEIN ALPHA SUBUNIT |
| | | | | | | GROUP I SIGNATURE |
| 1100 | PR00441F | 11.65 | 6.850e−14 | 331-341 | PR00441 | G-PROTEIN ALPHA SUBUNIT |
| | | | | | | GROUP I SIGNATURE |
| 1100 | PR00441G | 11.67 | 2.543e−13 | 347-361 | PR00441 | G-PROTEIN ALPHA SUBUNIT |
| | | | | | | GROUP I SIGNATURE |
| 1100 | PR00442A | 6.46 | 2.385e−14 | 88-103 | PR00442 | G-PROTEIN ALPHA SUBUNIT |
| | | | | | | GROUP Q SIGNATURE |
| 1100 | PR00442B | 7.17 | 1.794e−18 | 221-244 | PR00442 | G-PROTEIN ALPHA SUBUNIT |
| | | | | | | GROUP Q SIGNATURE |
| 1100 | PR00442C | 17.61 | 9.654e−20 | 250-267 | PR00442 | G-PROTEIN ALPHA SUBUNIT |
| | | | | | | GROUP Q SIGNATURE |
| 1100 | PR00442D | 9.29 | 8.071e−18 | 272-300 | PR00442 | G-PROTEIN ALPHA SUBUNIT |
| | | | | | | GROUP Q SIGNATURE |
| 1100 | PR00442E | 7.23 | 1.106e−11 | 319-327 | PR00442 | G-PROTEIN ALPHA SUBUNIT |
| | | | | | | GROUP Q SIGNATURE |
| 1100 | PR00443A | 15.16 | 8.313e−18 | 88-103 | PR00443 | G-PROTEIN ALPHA SUBUNIT |
| | | | | | | GROUP S SIGNATURE |
| 1100 | PR00443C | 14.35 | 7.446e−16 | 221-243 | PR00443 | G-PROTEIN ALPHA SUBUNIT |
| | | | | | | GROUP S SIGNATURE |
| 1100 | PR00443D | 13.15 | 3.361e−17 | 250-267 | PR00443 | G-PROTEIN ALPHA SUBUNIT |
| | | | | | | GROUP S SIGNATURE |
| 1100 | PR00443E | 11.98 | 7.513e−17 | 272-300 | PR00443 | G-PROTEIN ALPHA SUBUNIT |
| | | | | | | GROUP S SIGNATURE |
| 1100 | PR00443F | 12.68 | 5.909e−10 | 319-327 | PR00443 | G-PROTEIN ALPHA SUBUNIT |
| | | | | | | GROUP S SIGNATURE |
| 1100 | PR00449A | 13.20 | 1.000e−08 | 87-108 | PR00449 | TRANSFORMING PROTEIN |
| | | | | | | P21 RAS SIGNATURE |
| 1101 | BL00166C | 18.93 | 4.600e−13 | 453-479 | BL00166 | Enoyl-CoA |
| | | | | | | hydratase/isomerase |
| | | | | | | proteins. |
| 1101 | BL00166D | 22.87 | 5.600e−16 | 504-539 | BL00166 | Enoyl-CoA |
| | | | | | | hydratase/isomerase |
| | | | | | | proteins. |
| 1101 | BL00598 | 14.45 | 6.625e−16 | 92-113 | BL00598 | Chromo domain proteins. |
| 1101 | PR00504B | 9.12 | 4.600e−13 | 86-100 | PR00504 | CHROMODOMAIN SIGNATURE |
| 1101 | PR00504C | 11.19 | 5.592e−09 | 101-113 | PR00504 | CHROMODOMAIN SIGNATURE |
| 1101 | PR00584E | 4.20 | 8.000e−09 | 126-133 | PR00584 | PROSTANOID EP3 RECEPTOR |
| | | | | | | TYPE 2 SIGNATURE |
| 1102 | BL00383A | 13.34 | 4.150e−14 | 926-940 | BL00383 | Tyrosine specific |
| | | | | | | protein phosphatases |
| | | | | | | proteins. |
| 1102 | BL00383B | 7.61 | 5.000e−09 | 951-959 | BL00383 | Tyrosine specific |
| | | | | | | protein phosphatases |
| | | | | | | proteins. |
| 1102 | BL00383C | 10.10 | 5.091e−12 | 981-991 | BL00383 | Tyrosine specific |
| | | | | | | protein phosphatases |
| | | | | | | proteins. |
| 1102 | BL00383D | 11.92 | 1.000e−14 | 1055-1067 | BL00383 | Tyrosine specific |
| | | | | | | protein phosphatases |
| | | | | | | proteins. |
| 1102 | BL00383D | 11.92 | 5.500e−09 | 1293-1305 | BL00383 | Tyrosine specific |
| | | | | | | protein phosphatases |
| | | | | | | proteins. |
| 1102 | BL00383E | 10.35 | 1.789e−09 | 1387-1397 | BL00383 | Tyrosine specific |
| | | | | | | protein phosphatases |
| | | | | | | proteins. |
| 1102 | BL00383E | 10.35 | 7.300e−14 | 1093-1103 | BL00383 | Tyrosine specific |
| | | | | | | protein phosphatases |
| | | | | | | proteins. |
| 1102 | BL00383F | 15.51 | 1.439e−09 | 1425-1440 | BL00383 | Tyrosine specific |
| | | | | | | protein phosphatases |
| | | | | | | proteins. |
| 1102 | BL00383F | 15.51 | 4.913e−14 | 1131-1146 | BL00383 | Tyrosine specific |
| | | | | | | protein phosphatases |
| | | | | | | proteins. |
| 1102 | BL00740A | 13.87 | 8.667e−13 | 36-48 | BL00740 | MAM domain proteins. |
| 1102 | BL00740B | 19.76 | 5.500e−16 | 167-187 | BL00740 | MAM domain proteins. |
| 1102 | BL00740C | 15.93 | 9.000e−11 | 681-691 | BL00740 | MAM domain proteins. |
| 1102 | BL00790H | 13.42 | 3.769e−09 | 498-523 | BL00790 | Receptor tyrosine |
| | | | | | | kinase class V |
| | | | | | | proteins. |
| 1102 | BL00790I | 20.01 | 2.607e−09 | 552-582 | BL00790 | Receptor tyrosine |
| | | | | | | kinase class V |
| | | | | | | proteins. |
| 1102 | PR00014C | 15.44 | 4.913e−09 | 331-349 | PR00014 | FIBRONECTIN TYPE III |
| | | | | | | REPEAT SIGNATURE |
| 1102 | PR00014C | 15.44 | 9.000e−11 | 541-559 | PR00014 | FIBRONECTIN TYPE III |
| | | | | | | REPEAT SIGNATURE |
| 1102 | PR00014D | 12.04 | 9.400e−11 | 559-573 | PR00014 | FIBRONECTIN TYPE III |
| | | | | | | REPEAT SIGNATURE |
| 1102 | PR00020A | 18.17 | 1.000e−17 | 34-52 | PR00020 | MAM DOMAIN SIGNATURE |
| 1102 | PR00020B | 15.52 | 4.971e−14 | 68-84 | PR00020 | MAM DOMAIN SIGNATURE |
| 1102 | PR00020C | 13.66 | 3.483e−13 | 95-106 | PR00020 | MAM DOMAIN SIGNATURE |
| 1102 | PR00020D | 12.70 | 2.543e−15 | 147-161 | PR00020 | MAM DOMAIN SIGNATURE |
| 1102 | PR00020E | 8.64 | 6.586e−13 | 165-178 | PR00020 | MAM DOMAIN SIGNATURE |
| 1102 | PR00700B | 16.80 | 4.162e−16 | 1257-1277 | PR00700 | PROTEIN TYROSINE |
| | | | | | | PHOSPHATASE SIGNATURE |
| 1102 | PR00700B | 16.80 | 6.400e−23 | 968-988 | PR00700 | PROTEIN TYROSINE |
| | | | | | | PHOSPHATASE SIGNATURE |
| 1102 | PR00700C | 13.17 | 3.077e−19 | 1051-1068 | PR00700 | PROTEIN TYROSINE |
| | | | | | | PHOSPHATASE SIGNATURE |
| 1102 | PR00700D | 12.47 | 7.136e−13 | 1384-1402 | PR00700 | PROTEIN TYROSINE |
| | | | | | | PHOSPHATASE SIGNATURE |
| 1102 | PR00700D | 12.47 | 7.158e−20 | 1090-1108 | PR00700 | PROTEIN TYROSINE |
| | | | | | | PHOSPHATASE SIGNATURE |
| 1102 | PR00700E | 17.57 | 9.550e−13 | 1121-1136 | PR00700 | PROTEIN TYROSINE |
| | | | | | | PHOSPHATASE SIGNATURE |
| 1102 | PR00700F | 11.18 | 6.824e−13 | 1137-1147 | PR00700 | PROTEIN TYROSINE |
| | | | | | | PHOSPHATASE SIGNATURE |
| 1104 | PF00855 | 13.75 | 1.000e−20 | 119-135 | PF00855 | PWWP domain proteins. |
| 1105 | BL50062B | 12.92 | 1.290e−12 | 280-290 | BL50062 | BCL2-like apoptosis |
| | | | | | | inhibitors (spans part |
| | | | | | | of BH3, BH1 and BH. |
| 1105 | BL50062C | 6.66 | 8.676e−09 | 325-333 | BL50062 | BCL2-like apoptosis |
| | | | | | | inhibitors (spans part |
| | | | | | | of BH3, BH1 and BH. |
| 1106 | BL00216B | 27.64 | 5.765e−35 | 123-172 | BL00216 | Sugar transport |
| | | | | | | proteins. |
| 1106 | PR00171A | 10.00 | 8.615e−13 | 24-34 | PR00171 | SUGAR TRANSPORTER |
| | | | | | | SIGNATURE |
| 1106 | PR00171B | 14.73 | 4.789e−17 | 124-143 | PR00171 | SUGAR TRANSPORTER |
| | | | | | | SIGNATURE |
| 1106 | PR00171C | 10.97 | 3.143e−13 | 283-293 | PR00171 | SUGAR TRANSPORTER |
| | | | | | | SIGNATURE |
| 1106 | PR00171D | 12.76 | 5.500e−19 | 370-391 | PR00171 | SUGAR TRANSPORTER |
| | | | | | | SIGNATURE |
| 1106 | PR00171E | 14.87 | 8.500e−13 | 393-405 | PR00171 | SUGAR TRANSPORTER |
| | | | | | | SIGNATURE |
| 1106 | PR00172A | 9.82 | 9.182e−26 | 273-294 | PR00172 | GLUCOSE TRANSPORTER |
| | | | | | | SIGNATURE |
| 1106 | PR00172B | 8.42 | 1.000e−23 | 308-329 | PR00172 | GLUCOSE TRANSPORTER |
| | | | | | | SIGNATURE |
| 1106 | PR00172C | 9.51 | 7.828e−19 | 339-359 | PR00172 | GLUCOSE TRANSPORTER |
| | | | | | | SIGNATURE |
| 1106 | PR00172D | 9.13 | 1.000e−25 | 370-393 | PR00172 | GLUCOSE TRANSPORTER |
| | | | | | | SIGNATURE |
| 1106 | PR00172E | 8.29 | 2.895e−21 | 403-421 | PR00172 | GLUCOSE TRANSPORTER |
| | | | | | | SIGNATURE |
| 1106 | PR00172F | 8.47 | 7.188e−20 | 433-453 | PR00172 | GLUCOSE TRANSPORTER |
| | | | | | | SIGNATURE |
| 1107 | PR00704A | 14.68 | 9.069e−20 | 14-37 | PR00704 | CALPAIN CYSTEINE |
| | | | | | | PROTEASE (C2) FAMILY |
| | | | | | | SIGNATURE |
| 1107 | PR00704B | 17.94 | 2.636e−22 | 54-76 | PR00704 | CALPAIN CYSTEINE |
| | | | | | | PROTEASE (C2) FAMILY |
| | | | | | | SIGNATURE |
| 1107 | PR00704C | 11.88 | 4.000e−16 | 78-94 | PR00704 | CALPAIN CYSTEINE |
| | | | | | | PROTEASE (C2) FAMILY |
| | | | | | | SIGNATURE |
| 1107 | PR00704D | 11.05 | 2.000e−30 | 120-145 | PR00704 | CALPAIN CYSTEINE |
| | | | | | | PROTEASE (C2) FAMILY |
| | | | | | | SIGNATURE |
| 1107 | PR00704E | 12.55 | 5.886e−25 | 150-173 | PR00704 | CALPAIN CYSTEINE |
| | | | | | | PROTEASE (C2) FAMILY |
| | | | | | | SIGNATURE |
| 1107 | PR00704F | 13.61 | 8.364e−25 | 175-202 | PR00704 | CALPAIN CYSTEINE |
| | | | | | | PROTEASE (C2) FAMILY |
| | | | | | | SIGNATURE |
| 1107 | PR00704G | 13.87 | 7.667e−24 | 322-343 | PR00704 | CALPAIN CYSTEINE |
| | | | | | | PROTEASE (C2) FAMILY |
| | | | | | | SIGNATURE |
| 1107 | PR00704H | 13.38 | 3.676e−19 | 373-390 | PR00704 | CALPAIN CYSTEINE |
| | | | | | | PROTEASE (C2) FAMILY |
| | | | | | | SIGNATURE |
| 1107 | PR00704I | 9.52 | 8.941e−26 | 464-492 | PR00704 | CALPAIN CYSTEINE |
| | | | | | | PROTEASE (C2) FAMILY |
| | | | | | | SIGNATURE |
| 1108 | BL00383E | 10.35 | 9.780e−12 | 402-412 | BL00383 | Tyrosine specific |
| | | | | | | protein phosphatases |
| | | | | | | proteins. |
| 1108 | BL00383F | 15.51 | 9.341e−09 | 239-254 | BL00383 | Tyrosine specific |
| | | | | | | protein phosphatases |
| | | | | | | proteins. |
| 1108 | PR00716C | 17.65 | 5.011e−09 | 147-167 | PR00716 | M-PHASE INDUCER |
| | | | | | | PHOSPHATASE SIGNATURE |
| 1112 | BL00657A | 19.39 | 8.091e−30 | 109-150 | BL00657 | Fork head domain |
| | | | | | | proteins. |
| 1112 | BL00657B | 22.27 | 7.750e−26 | 157-199 | BL00657 | Fork head domain |
| | | | | | | proteins. |
| 1112 | PR00053A | 8.86 | 8.050e−09 | 109-122 | PR00053 | FORK HEAD DOMAIN |
| | | | | | | SIGNATURE |
| 1112 | PR00053B | 13.70 | 7.698e−13 | 130-147 | PR00053 | FORK HEAD DOMAIN |
| | | | | | | SIGNATURE |
| 1112 | PR00053C | 12.80 | 2.125e−19 | 158-175 | PR00053 | FORK HEAD DOMAIN |
| | | | | | | SIGNATURE |
| 1113 | BL00484B | 9.04 | 3.400e−12 | 189-202 | BL00484 | Thyroglobulin type-1 |
| | | | | | | repeat proteins |
| | | | | | | proteins. |
| 1113 | BL00484C | 17.01 | 9.206e−12 | 208-222 | BL00484 | Thyroglobulin type-1 |
| | | | | | | repeat proteins |
| | | | | | | proteins. |
| 1117 | BL00222A | 11.34 | 5.125e−09 | 92-102 | BL00222 | Insulin-like growth |
| | | | | | | factor binding |
| | | | | | | proteins. |
| 1117 | BL00222B | 11.09 | 1.375e−16 | 111-126 | BL00222 | Insulin-like growth |
| | | | | | | factor binding |
| | | | | | | proteins. |
| 1117 | BL00222C | 22.97 | 1.667e−26 | 261-288 | BL00222 | Insulin-like growth |
| | | | | | | factor binding |
| | | | | | | proteins. |
| 1117 | BL00484B | 9.04 | 3.118e−17 | 262-275 | BL00484 | Thyroglobulin type-1 |
| | | | | | | repeat proteins |
| | | | | | | proteins. |
| 1117 | BL00484C | 17.01 | 4.536e−13 | 286-300 | BL00484 | Thyroglobulin type-1 |
| | | | | | | repeat proteins |
| | | | | | | proteins. |
| 1119 | BL00022B | 7.54 | 7.750e−10 | 203-209 | BL00022 | EGF-like domain |
| | | | | | | proteins. |
| 1119 | BL01187A | 9.98 | 2.714e−10 | 175-186 | BL01187 | Calcium-binding EGF- |
| | | | | | | like domain proteins |
| | | | | | | pattern proteins. |
| 1119 | BL01187A | 9.98 | 4.750e−09 | 216-227 | BL01187 | Calcium-binding EGF- |
| | | | | | | like domain proteins |
| | | | | | | pattern proteins. |
| 1119 | BL01187A | 9.98 | 4.750e−09 | 46-57 | BL01187 | Calcium-binding EGF- |
| | | | | | | like domain proteins |
| | | | | | | pattern proteins. |
| 1119 | BL01187B | 12.04 | 3.000e−12 | 272-287 | BL01187 | Calcium-binding EGF- |
| | | | | | | like domain proteins |
| | | | | | | pattern proteins. |
| 1119 | BL01187B | 12.04 | 5.304e−13 | 194-209 | BL01187 | Calcium-binding EGF- |
| | | | | | | like domain proteins |
| | | | | | | pattern proteins. |
| 1119 | BL01187B | 12.04 | 5.500e−14 | 232-247 | BL01187 | Calcium-binding EGF- |
| | | | | | | like domain proteins |
| | | | | | | pattern proteins. |
| 1119 | BL01187B | 12.04 | 9.400e−11 | 313-328 | BL01187 | Calcium-binding EGF- |
| | | | | | | like domain proteins |
| | | | | | | pattern proteins. |
| 1119 | PD02283C | 17.54 | 8.988e−09 | 58-85 | PD02283 | PROTEIN SPORULATION |
| | | | | | | REPEAT PRECU. |
| 1119 | PR00010C | 11.16 | 1.273e−10 | 237-247 | PR00010 | TYPE II EGF-LIKE |
| | | | | | | SIGNATURE |
| 1119 | PR00010C | 11.16 | 4.667e−11 | 199-209 | PR00010 | TYPE II EGF-LIKE |
| | | | | | | SIGNATURE |
| 1119 | PR00907B | 11.29 | 6.260e−10 | 309-325 | PR00907 | THROMBOMODULIN |
| | | | | | | SIGNATURE |
| 1119 | PR00907G | 11.63 | 3.676e−09 | 194-220 | PR00907 | THROMBOMODULIN |
| | | | | | | SIGNATURE |
| 1119 | PR00907G | 11.63 | 7.368e−10 | 232-258 | PR00907 | THROMBOMODULIN |
| | | | | | | SIGNATURE |
| 1120 | BL50017B | 17.60 | 5.263e−11 | 4112-4127 | BL50017 | Death domain proteins |
| | | | | | | profile. |
| 1120 | DM00784B | 17.87 | 6.311e−09 | 3961-3985 | DM00784 | APILLOMAVIRUS E4 |
| | | | | | | PROTEIN. |
| 1120 | DM00784B | 17.87 | 7.197e−09 | 3964-3988 | DM00784 | APILLOMAVIRUS E4 |
| | | | | | | PROTEIN. |
| 1120 | DM00784B | 17.87 | 9.705e−09 | 3968-3992 | DM00784 | APILLOMAVIRUS E4 |
| | | | | | | PROTEIN. |
| 1120 | PD00078B | 13.14 | 6.478e−09 | 701-713 | PD00078 | REPEAT PROTEIN ANK |
| | | | | | | NUCLEAR ANKYR. |
| 1120 | PD01364B | 13.94 | 2.421e−09 | 3974-3989 | PD01364 | MUCIN GLYCOPROTEIN |
| | | | | | | PRECURSOR MEM. |
| 1120 | PD01364B | 13.94 | 3.368e−09 | 3969-3984 | PD01364 | MUCIN GLYCOPROTEIN |
| | | | | | | PRECURSOR MEM. |
| 1120 | PD01364B | 13.94 | 6.684e−09 | 3967-3982 | PD01364 | MUCIN GLYCOPROTEIN |
| | | | | | | PRECURSOR MEM. |
| 1120 | PD01364B | 13.94 | 8.364e−10 | 3970-3985 | PD01364 | MUCIN GLYCOPROTEIN |
| | | | | | | PRECURSOR MEM. |
| 1120 | PF00023A | 16.03 | 1.000e−09 | 118-133 | PF00023 | Ank repeat proteins. |
| 1120 | PF00023A | 16.03 | 2.875e−10 | 85-100 | PF00023 | Ank repeat proteins. |
| 1120 | PF00023A | 16.03 | 3.000e−12 | 477-492 | PF00023 | Ank repeat proteins. |
| 1120 | PF00023A | 16.03 | 4.214e−09 | 576-591 | PF00023 | Ank repeat proteins. |
| 1120 | PF00023A | 16.03 | 4.375e−10 | 444-459 | PF00023 | Ank repeat proteins. |
| 1120 | PF00023A | 16.03 | 6.143e−09 | 279-294 | PF00023 | Ank repeat proteins. |
| 1120 | PF00023A | 16.03 | 7.000e−10 | 378-393 | PF00023 | Ank repeat proteins. |
| 1120 | PF00023A | 16.03 | 7.000e−11 | 675-690 | PF00023 | Ank repeat proteins. |
| 1120 | PF00023A | 16.03 | 9.679e−09 | 312-327 | PF00023 | Ank repeat proteins. |
| 1120 | PF00023B | 14.20 | 5.000e−10 | 704-713 | PF00023 | Ank repeat proteins. |
| 1120 | PF00023B | 14.20 | 5.500e−09 | 572-581 | PF00023 | Ank repeat proteins. |
| 1120 | PF00023B | 14.20 | 8.500e−10 | 407-416 | PF00023 | Ank repeat proteins. |
| 1120 | PF00023B | 14.20 | 8.773e−09 | 605-614 | PF00023 | Ank repeat proteins. |
| 1120 | PF00023B | 14.20 | 9.000e−10 | 242-251 | PF00023 | Ank repeat proteins. |
| 1120 | PF00023B | 14.20 | 9.182e−09 | 770-779 | PF00023 | Ank repeat proteins. |
| 1120 | PF00791A | 27.85 | 1.000e−40 | 168-222 | PF00791 | Domain present in ZO-1 |
| | | | | | | and Unc5-like netrin |
| | | | | | | receptors. |
| 1120 | PF00791B | 28.49 | 1.000e−40 | 279-333 | PF00791 | Domain present in ZO-1 |
| | | | | | | and Unc5-like netrin |
| | | | | | | receptors. |
| 1120 | PF00791B | 28.49 | 1.205e−14 | 118-172 | PF00791 | Domain present in ZO-1 |
| | | | | | | and Unc5-like netrin |
| | | | | | | receptors. |
| 1120 | PF00791B | 28.49 | 1.692e−13 | 151-205 | PF00791 | Domain present in ZO-1 |
| | | | | | | and Unc5-like netrin |
| | | | | | | receptors. |
| 1120 | PF00791B | 28.49 | 2.367e−17 | 411-465 | PF00791 | Domain present in ZO-1 |
| | | | | | | and Unc5-like netrin |
| | | | | | | receptors. |
| 1120 | PF00791B | 28.49 | 3.368e−12 | 85-139 | PF00791 | Domain present in ZO-1 |
| | | | | | | and Unc5-like netrin |
| | | | | | | receptors. |
| 1120 | PF00791B | 28.49 | 4.221e−12 | 213-267 | PF00791 | Domain present in ZO-1 |
| | | | | | | and Unc5-like netrin |
| | | | | | | receptors. |
| 1120 | PF00791B | 28.49 | 4.462e−13 | 609-663 | PF00791 | Domain present in ZO-1 |
| | | | | | | and Unc5-like netrin |
| | | | | | | receptors. |
| 1120 | PF00791B | 28.49 | 4.987e−17 | 477-531 | PF00791 | Domain present in ZO-1 |
| | | | | | | and Unc5-like netrin |
| | | | | | | receptors. |
| 1120 | PF00791B | 28.49 | 5.500e−14 | 543-597 | PF00791 | Domain present in ZO-1 |
| | | | | | | and Unc5-like netrin |
| | | | | | | receptors. |
| 1120 | PF00791B | 28.49 | 5.500e−16 | 246-300 | PF00791 | Domain present in ZO-1 |
| | | | | | | and Unc5-like netrin |
| | | | | | | receptors. |
| 1120 | PF00791B | 28.49 | 6.637e−13 | 345-399 | PF00791 | Domain present in ZO-1 |
| | | | | | | and Unc5-like netrin |
| | | | | | | receptors. |
| 1120 | PF00791B | 28.49 | 6.874e−12 | 708-762 | PF00791 | Domain present in ZO-1 |
| | | | | | | and Unc5-like netrin |
| | | | | | | receptors. |
| 1120 | PF00791B | 28.49 | 6.927e−16 | 741-795 | PF00791 | Domain present in ZO-1 |
| | | | | | | and Unc5-like netrin |
| | | | | | | receptors. |
| 1120 | PF00791B | 28.49 | 6.932e−14 | 642-696 | PF00791 | Domain present in ZO-1 |
| | | | | | | and Unc5-like netrin |
| | | | | | | receptors. |
| 1120 | PF00791B | 28.49 | 7.136e−14 | 378-432 | PF00791 | Domain present in ZO-1 |
| | | | | | | and Unc5-like netrin |
| | | | | | | receptors. |
| 1120 | PF00791B | 28.49 | 7.442e−12 | 444-498 | PF00791 | Domain present in ZO-1 |
| | | | | | | and Unc5-like netrin |
| | | | | | | receptors. |
| 1120 | PF00791B | 28.49 | 7.459e−15 | 312-366 | PF00791 | Domain present in ZO-1 |
| | | | | | | and Unc5-like netrin |
| | | | | | | receptors. |
| 1120 | PF00791B | 28.49 | 7.835e−17 | 510-564 | PF00791 | Domain present in ZO-1 |
| | | | | | | and Unc5-like netrin |
| | | | | | | receptors. |
| 1120 | PF00791B | 28.49 | 8.354e−16 | 675-729 | PF00791 | Domain present in ZO-1 |
| | | | | | | and Unc5-like netrin |
| | | | | | | receptors. |
| 1120 | PF00791B | 28.49 | 8.364e−14 | 576-630 | PF00791 | Domain present in ZO-1 |
| | | | | | | and Unc5-like netrin |
| | | | | | | receptors. |
| 1120 | PF00791C | 20.98 | 1.000e−10 | 132-170 | PF00791 | Domain present in ZO-1 |
| | | | | | | and Unc5-like netrin |
| | | | | | | receptors. |
| 1120 | PF00791C | 20.98 | 1.000e−10 | 326-364 | PF00791 | Domain present in ZO-1 |
| | | | | | | and Unc5-like netrin |
| | | | | | | receptors. |
| 1120 | PF00791C | 20.98 | 1.818e−12 | 557-595 | PF00791 | Domain present in ZO-1 |
| | | | | | | and Unc5-like netrin |
| | | | | | | receptors. |
| 1120 | PF00791C | 20.98 | 1.882e−09 | 99-137 | PF00791 | Domain present in ZO-1 |
| | | | | | | and Unc5-like netrin |
| | | | | | | receptors. |
| 1120 | PF00791C | 20.98 | 2.432e−12 | 392-430 | PF00791 | Domain present in ZO-1 |
| | | | | | | and Unc5-like netrin |
| | | | | | | receptors. |
| 1120 | PF00791C | 20.98 | 3.311e−16 | 623-661 | PF00791 | Domain present in ZO-1 |
| | | | | | | and Unc5-like netrin |
| | | | | | | receptors. |
| 1120 | PF00791C | 20.98 | 3.961e−15 | 227-265 | PF00791 | Domain present in ZO-1 |
| | | | | | | and Unc5-like netrin |
| | | | | | | receptors. |
| 1120 | PF00791C | 20.98 | 5.147e−09 | 491-529 | PF00791 | Domain present in ZO-1 |
| | | | | | | and Unc5-like netrin |
| | | | | | | receptors. |
| 1120 | PF00791C | 20.98 | 6.318e−12 | 689-727 | PF00791 | Domain present in ZO-1 |
| | | | | | | and Unc5-like netrin |
| | | | | | | receptors. |
| 1120 | PF00791C | 20.98 | 6.747e−13 | 260-298 | PF00791 | Domain present in ZO-1 |
| | | | | | | and Unc5-like netrin |
| | | | | | | receptors. |
| 1120 | PF00791C | 20.98 | 6.778e−14 | 722-760 | PF00791 | Domain present in ZO-1 |
| | | | | | | and Unc5-like netrin |
| | | | | | | receptors. |
| 1120 | PF00791C | 20.98 | 7.163e−11 | 293-331 | PF00791 | Domain present in ZO-1 |
| | | | | | | and Unc5-like netrin |
| | | | | | | receptors. |
| 1120 | PF00791C | 20.98 | 7.216e−10 | 359-397 | PF00791 | Domain present in ZO-1 |
| | | | | | | and Unc5-like netrin |
| | | | | | | receptors. |
| 1120 | PF00791C | 20.98 | 7.239e−12 | 755-793 | PF00791 | Domain present in ZO-1 |
| | | | | | | and Unc5-like netrin |
| | | | | | | receptors. |
| 1120 | PF00791C | 20.98 | 7.265e−09 | 165-203 | PF00791 | Domain present in ZO-1 |
| | | | | | | and Unc5-like netrin |
| | | | | | | receptors. |
| 1120 | PF00791C | 20.98 | 7.706e−26 | 590-628 | PF00791 | Domain present in ZO-1 |
| | | | | | | and Unc5-like netrin |
| | | | | | | receptors. |
| 1120 | PF00791C | 20.98 | 8.826e−11 | 524-562 | PF00791 | Domain present in ZO-1 |
| | | | | | | and Unc5-like netrin |
| | | | | | | receptors. |
| 1120 | PF00791C | 20.98 | 9.536e−10 | 425-463 | PF00791 | Domain present in ZO-1 |
| | | | | | | and Unc5-like netrin |
| | | | | | | receptors. |
| 1120 | PF00791D | 20.37 | 1.675e−36 | 1110-1152 | PF00791 | Domain present in ZO-1 |
| | | | | | | and Unc5-like netrin |
| | | | | | | receptors. |
| 1120 | PF00791E | 25.76 | 1.000e−40 | 1178-1230 | PF00791 | Domain present in ZO-1 |
| | | | | | | and Unc5-like netrin |
| | | | | | | receptors. |
| 1120 | PF00791F | 18.25 | 2.313e−19 | 1399-1423 | PF00791 | Domain present in ZO-1 |
| | | | | | | and Unc5-like netrin |
| | | | | | | receptors. |
| 1120 | PF00791G | 12.16 | 2.887e−11 | 1436-1451 | PF00791 | Domain present in ZO-1 |
| | | | | | | and Unc5-like netrin |
| | | | | | | receptors. |
| 1121 | BL00107A | 18.39 | 4.938e−21 | 459-489 | BL00107 | Protein kinases ATP- |
| | | | | | | binding region |
| | | | | | | proteins. |
| 1121 | BL00239B | 25.15 | 8.967e−19 | 394-441 | BL00239 | Receptor tyrosine |
| | | | | | | kinase class II |
| | | | | | | proteins. |
| 1121 | BL00239C | 18.75 | 9.169e−11 | 446-468 | BL00239 | Receptor tyrosine |
| | | | | | | kinase class II |
| | | | | | | proteins. |
| 1121 | BL00239E | 17.14 | 1.802e−10 | 516-565 | BL00239 | Receptor tyrosine |
| | | | | | | kinase class II |
| | | | | | | proteins. |
| 1121 | BL00239F | 28.15 | 6.082e−09 | 571-615 | BL00239 | Receptor tyrosine |
| | | | | | | kinase class II |
| | | | | | | proteins. |
| 1121 | BL00240E | 11.56 | 3.813e−11 | 445-482 | BL00240 | Receptor tyrosine |
| | | | | | | kinase class III |
| | | | | | | proteins. |
| 1121 | BL00240F | 17.74 | 3.172e−09 | 515-562 | BL00240 | Receptor tyrosine |
| | | | | | | kinase class III |
| | | | | | | proteins. |
| 1121 | BL00478B | 14.79 | 1.643e−13 | 51-65 | BL00478 | LIM domain proteins. |
| 1121 | BL00478B | 14.79 | 7.800e−11 | 109-123 | BL00478 | LIM domain proteins. |
| 1121 | BL00790M | 8.74 | 5.382e−13 | 450-471 | BL00790 | Receptor tyrosine |
| | | | | | | kinase class V |
| | | | | | | proteins. |
| 1121 | BL50001B | 17.40 | 1.000e−08 | 456-476 | BL50001 | Src homology 2 (SH2) |
| | | | | | | domain proteins |
| | | | | | | profile. |
| 1121 | PD00289 | 9.97 | 8.200e−11 | 219-232 | PD00289 | PROTEIN SH3 DOMAIN |
| | | | | | | REPEAT PRESYNA. |
| 1121 | PF00595 | 13.40 | 3.000e−11 | 216-226 | PF00595 | PDZ domain proteins |
| | | | | | | (Also known as DHR or |
| | | | | | | GLGF). |
| 1121 | PR00109A | 15.00 | 8.839e−10 | 423-436 | PR00109 | TYROSINE KINASE |
| | | | | | | CATALYTIC DOMAIN |
| | | | | | | SIGNATURE |
| 1121 | PR00109B | 12.27 | 7.511e−13 | 459-477 | PR00109 | TYROSINE KINASE |
| | | | | | | CATALYTIC DOMAIN |
| | | | | | | SIGNATURE |
| 1121 | PR00109E | 14.41 | 7.894e−10 | 590-612 | PR00109 | TYROSINE KINASE |
| | | | | | | CATALYTIC DOMAIN |
| | | | | | | SIGNATURE |
| 1123 | BL00280 | 24.61 | 3.172e−33 | 36-79 | BL00280 | Pancreatic trypsin |
| | | | | | | inhibitor (Kunitz) |
| | | | | | | family proteins. |
| 1123 | PR00759A | 14.51 | 3.880e−10 | 26-40 | PR00759 | BASIC PROTEASE (KUNITZ- |
| | | | | | | TYPE) INHIBITOR FAMILY |
| | | | | | | SIGNATURE |
| 1123 | PR00759B | 11.26 | 6.400e−13 | 54-64 | PR00759 | BASIC PROTEASE (KUNITZ- |
| | | | | | | TYPE) INHIBITOR FAMILY |
| | | | | | | SIGNATURE |
| 1123 | PR00759C | 14.15 | 2.364e−12 | 64-79 | PR00759 | BASIC PROTEASE (KUNITZ- |
| | | | | | | TYPE) INHIBITOR FAMILY |
| | | | | | | SIGNATURE |
| 1125 | PR00790I | 14.97 | 1.818e−32 | 77-100 | PR00790 | PEPTIDYL-GLYCINE ALPHA- |
| | | | | | | AMIDATING MONOOXYGENASE |
| | | | | | | SIGNATURE |
| 1126 | DM00406 | 7.73 | 2.000e−09 | 637-649 | DM00406 | GLIADIN. |
| 1126 | PD01234A | 11.06 | 1.450e−09 | 638-648 | PD01234 | PROTEIN NUCLEAR |
| | | | | | | BROMODOMAIN TRANS. |
| 1126 | PF00806B | 11.32 | 4.176e−12 | 980-989 | PF00806 | Pumilio-family RNA |
| | | | | | | binding domain proteins |
| | | | | | | (aka PUM-HD, Pumilio |
| | | | | | | homol. |
| 1126 | PF00806C | 7.81 | 2.452e−10 | 1131-1139 | PF00806 | Pumilio-family RNA |
| | | | | | | binding domain proteins |
| | | | | | | (aka PUM-HD, Pumilio |
| 1126 | PF00806C | 7.81 | 5.263e−11 | 1052-1060 | PF00806 | Pumilio-family RNA |
| | | | | | | binding domain proteins |
| | | | | | | (aka PUM-HD, Pumilio |
| | | | | | | homol. |
| 1126 | PF00806C | 7.81 | 7.632e−09 | 908-916 | PF00806 | Pumilio-family RNA |
| | | | | | | binding domain proteins |
| | | | | | | (aka PUM-HD, Pumilio |
| | | | | | | homol. |
| 1126 | PR00308C | 3.83 | 9.649e−09 | 490-499 | PR00308 | TYPE I ANTIFREEZE |
| | | | | | | PROTEIN SIGNATURE |
| 1130 | PD00301B | 5.49 | 5.154e−09 | 137-147 | PD00301 | PROTEIN REPEAT MUSCLE |
| | | | | | | CALCIUM-BI. |
| 1130 | PR00796I | 8.96 | 1.517e−09 | 98-123 | PR00796 | VIRAL SPIKE |
| | | | | | | GLYCOPROTEIN PRECURSOR |
| | | | | | | SIGNATURE |
| 1132 | BL00018 | 7.41 | 8.435e−09 | 28-40 | BL00018 | EF-hand calcium-binding |
| | | | | | | domain proteins. |
| 1133 | BL00203 | 13.94 | 7.415e−10 | 2460-2505 | BL00203 | Vertebrate |
| | | | | | | metallothioneins |
| | | | | | | proteins. |
| 1133 | BL01185C | 15.86 | 4.506e−10 | 2043-2081 | BL01185 | C-terminal cystine knot |
| | | | | | | proteins. |
| 1133 | BL01185C | 15.86 | 9.065e−10 | 960-998 | BL01185 | C-terminal cystine knot |
| | | | | | | proteins. |
| 1133 | BL01185D | 23.45 | 8.800e−16 | 2860-2912 | BL01185 | C-terminal cystine knot |
| | | | | | | proteins. |
| 1133 | BL01208B | 15.83 | 4.938e−10 | 775-789 | BL01208 | VWFC domain proteins. |
| 1133 | PD02576A | 27.60 | 1.000e−40 | 932-980 | PD02576 | PRECURSOR GLYCOPROTEIN |
| | | | | | | SIGNAL CELL. |
| 1133 | PD02576A | 27.60 | 4.857e−27 | 453-501 | PD02576 | PRECURSOR GLYCOPROTEIN |
| | | | | | | SIGNAL CELL. |
| 1133 | PD02576A | 27.60 | 7.882e−10 | 2534-2582 | PD02576 | PRECURSOR GLYCOPROTEIN |
| | | | | | | SIGNAL CELL. |
| 1133 | PD02576B | 25.43 | 1.667e−22 | 1495-1522 | PD02576 | PRECURSOR GLYCOPROTEIN |
| | | | | | | SIGNAL CELL. |
| 1133 | PF00094A | 11.09 | 4.333e−13 | 476-485 | PF00094 | von Willebrand factor |
| | | | | | | type D domain proteins. |
| 1133 | PF00094B | 10.43 | 3.647e−23 | 624-641 | PF00094 | von Willebrand factor |
| | | | | | | type D domain proteins. |
| 1133 | PF00094B | 10.43 | 8.440e−11 | 1096-1113 | PF00094 | von Willebrand factor |
| | | | | | | type D domain proteins. |
| 1133 | PF00094B | 10.43 | 9.571e−14 | 262-279 | PF00094 | von Willebrand factor |
| | | | | | | type D domain proteins. |
| 1133 | PF00094C | 12.88 | 5.500e−15 | 835-844 | PF00094 | von Willebrand factor |
| | | | | | | type D domain proteins. |
| 1133 | PR00453A | 12.79 | 4.656e−13 | 1382-1399 | PR00453 | VON WILLEBRAND FACTOR |
| | | | | | | TYPE A DOMAIN SIGNATURE |
| 1133 | PR00453A | 12.79 | 5.696e−10 | 1603-1620 | PR00453 | VON WILLEBRAND FACTOR |
| | | | | | | TYPE A DOMAIN SIGNATURE |
| 1133 | PR00453B | 14.65 | 5.135e−11 | 1642-1656 | PR00453 | VON WILLEBRAND FACTOR |
| | | | | | | TYPE A DOMAIN SIGNATURE |
| 1134 | PR00421B | 11.40 | 7.600e−09 | 94-103 | PR00421 | THIOREDOXIN FAMILY |
| | | | | | | SIGNATURE |
| 1135 | DM00215 | 19.43 | 5.729e−09 | 37-69 | DM00215 | PROLINE-RICH PROTEIN 3. |
| 1137 | BL00107A | 18.39 | 1.000e−20 | 197-227 | BL00107 | Protein kinases ATP- |
| | | | | | | binding region |
| | | | | | | proteins. |
| 1137 | BL00107B | 13.31 | 2.286e−10 | 268-283 | BL00107 | Protein kinases ATP- |
| | | | | | | binding region |
| | | | | | | proteins. |
| 1137 | BL00239B | 25.15 | 4.097e−09 | 124-171 | BL00239 | Receptor tyrosine |
| | | | | | | kinase class II |
| | | | | | | proteins. |
| 1137 | BL00240E | 11.56 | 5.371e−09 | 183-220 | BL00240 | Receptor tyrosine |
| | | | | | | kinase class III |
| | | | | | | proteins. |
| 1137 | BL00479C | 12.01 | 3.500e−09 | 246-258 | BL00479 | Phorbol esters/ |
| | | | | | | diacylglycerol binding |
| | | | | | | domain proteins. |
| 1137 | PR00109B | 12.27 | 3.471e−12 | 197-215 | PR00109 | TYROSINE KINASE |
| | | | | | | CATALYTIC DOMAIN |
| | | | | | | SIGNATURE |
| 1137 | PR00109D | 17.04 | 3.441e−10 | 269-291 | PR00109 | TYROSINE KINASE |
| | | | | | | CATALYTIC DOMAIN |
| | | | | | | SIGNATURE |
| 1138 | BL00297A | 17.58 | 2.895e−35 | 86-122 | BL00297 | Heat shock hsp70 |
| | | | | | | proteins family |
| | | | | | | proteins. |
| 1138 | BL00297B | 22.91 | 1.000e−40 | 126-174 | BL00297 | Heat shock hsp70 |
| | | | | | | proteins family |
| | | | | | | proteins. |
| 1138 | BL00297C | 9.51 | 1.000e−40 | 211-262 | BL00297 | Heat shock hsp70 |
| | | | | | | proteins family |
| | | | | | | proteins. |
| 1138 | BL00297D | 11.95 | 3.813e−40 | 270-309 | BL00297 | Heat shock hsp70 |
| | | | | | | proteins family |
| | | | | | | proteins. |
| 1138 | BL00297E | 18.56 | 6.478e−32 | 371-414 | BL00297 | Heat shock hsp70 |
| | | | | | | proteins family |
| | | | | | | proteins. |
| 1138 | BL00297F | 11.84 | 4.750e−25 | 434-461 | BL00297 | Heat shock hsp70 |
| | | | | | | proteins family |
| | | | | | | proteins. |
| 1138 | BL00297G | 21.87 | 1.000e−40 | 464-518 | BL00297 | Heat shock hsp70 |
| | | | | | | proteins family |
| | | | | | | proteins. |
| 1138 | BL00297H | 15.46 | 1.000e−40 | 527-580 | BL00297 | Heat shock hsp70 |
| | | | | | | proteins family |
| | | | | | | proteins. |
| 1138 | BL00933A | 17.50 | 7.857e−09 | 84-107 | BL00933 | FGGY family of |
| | | | | | | carbohydrate kinases |
| | | | | | | proteins. |
| 1138 | PR00301A | 14.84 | 3.455e−15 | 84-97 | PR00301 | 70 KD HEAT SHOCK |
| | | | | | | PROTEIN SIGNATURE |
| 1138 | PR00301B | 15.36 | 7.000e−15 | 112-124 | PR00301 | 70 KD HEAT SHOCK |
| | | | | | | PROTEIN SIGNATURE |
| 1138 | PR00301C | 8.62 | 3.903e−09 | 135-143 | PR00301 | 70 KD HEAT SHOCK |
| | | | | | | PROTEIN SIGNATURE |
| 1138 | PR00301D | 15.51 | 5.500e−27 | 219-239 | PR00301 | 70 KD HEAT SHOCK |
| | | | | | | PROTEIN SIGNATURE |
| 1138 | PR00301E | 8.11 | 1.900e−11 | 278-288 | PR00301 | 70 KD HEAT SHOCK |
| | | | | | | PROTEIN SIGNATURE |
| 1138 | PR00301F | 13.98 | 4.240e−18 | 410-426 | PR00301 | 70 KD HEAT SHOCK |
| | | | | | | PROTEIN SIGNATURE |
| 1138 | PR00301G | 13.78 | 9.609e−22 | 441-461 | PR00301 | 70 KD HEAT SHOCK |
| | | | | | | PROTEIN SIGNATURE |
| 1138 | PR00301H | 8.81 | 2.688e−22 | 464-483 | PR00301 | 70 KD HEAT SHOCK |
| | | | | | | PROTEIN SIGNATURE |
| 1138 | PR00301I | 12.76 | 7.500e−21 | 545-561 | PR00301 | 70 KD HEAT SHOCK |
| | | | | | | PROTEIN SIGNATURE |
| 1139 | BL00960A | 10.98 | 5.304e−12 | 64-75 | BL00960 | BTG1 family proteins. |
| 1139 | BL00960B | 24.47 | 1.000e−40 | 84-128 | BL00960 | BTG1 family proteins. |
| 1139 | BL00960C | 12.68 | 3.647e−26 | 148-169 | BL00960 | BTG1 family proteins. |
| 1139 | PD01234B | 15.53 | 9.679e−09 | 36-53 | PD01234 | PROTEIN NUCLEAR |
| | | | | | | BROMODOMAIN TRANS. |
| 1139 | PR00310A | 11.17 | 7.000e−27 | 66-90 | PR00310 | ANTI-PROLIFERATIVE |
| | | | | | | PROTEIN BTG1 FAMILY |
| | | | | | | SIGNATURE |
| 1139 | PR00310B | 10.59 | 4.000e−39 | 91-120 | PR00310 | ANTI-PROLIFERATIVE |
| | | | | | | PROTEIN BTG1 FAMILY |
| | | | | | | SIGNATURE |
| 1139 | PR00310C | 12.74 | 5.286e−35 | 121-150 | PR00310 | ANTI-PROLIFERATIVE |
| | | | | | | PROTEIN BTG1 FAMILY |
| | | | | | | SIGNATURE |
| 1139 | PR00310D | 9.10 | 1.540e−37 | 151-180 | PR00310 | ANTI-PROLIFERATIVE |
| | | | | | | PROTEIN BTG1 FAMILY |
| | | | | | | SIGNATURE |
| 1139 | PR00310E | 13.58 | 6.914e−24 | 197-216 | PR00310 | ANTI-PROLIFERATIVE |
| | | | | | | PROTIN BTG1 FAMILY |
| | | | | | | SIGNATURE |
| 1141 | BL00547A | 19.15 | 7.900e−22 | 89-115 | BL00547 | Transglutaminases |
| | | | | | | proteins. |
| 1141 | BL00547B | 29.05 | 1.000e−40 | 209-259 | BL00547 | Transglutaminases |
| | | | | | | proteins. |
| 1141 | BL00547C | 20.72 | 2.029e−29 | 281-318 | BL00547 | Transglutaminases |
| | | | | | | proteins. |
| 1141 | BL00547D | 20.72 | 1.000e−40 | 338-382 | BL00547 | Transglutaminases |
| | | | | | | proteins. |
| 1141 | BL00547E | 17.36 | 4.667e−37 | 396-430 | BL00547 | Transglutaminases |
| | | | | | | proteins. |
| 1141 | BL00547F | 20.84 | 9.700e−28 | 440-471 | BL00547 | Transglutaminases |
| | | | | | | proteins. |
| 1141 | BL00547G | 17.29 | 4.971e−31 | 489-526 | BL00547 | Transglutaminases |
| | | | | | | proteins. |
| 1141 | BL00547H | 14.36 | 9.471e−16 | 681-701 | BL00547 | Transglutaminases |
| | | | | | | proteins. |
| 1141 | PR00445A | 9.20 | 6.824e−09 | 356-363 | PR00445 | HUPF/HYPC HYDROGENASE |
| | | | | | | EXPRESSION/SYNTHESIS |
| | | | | | | FAMILY SIGNATURE |
| 1143 | BL00224B | 16.94 | 9.673e−09 | 589-641 | BL00224 | Clathrin light chain |
| | | | | | | proteins. |
| 1143 | BL00686 | 13.91 | 6.581e−09 | 820-868 | BL00686 | CBF-B/NF-YA subunit |
| | | | | | | proteins. |
| 1143 | PF00992A | 16.67 | 1.474e−09 | 819-853 | PF00992 | Troponin. |
| 1143 | PF00992A | 16.67 | 2.184e−09 | 798-832 | PF00992 | Troponin. |
| 1143 | PF00992A | 16.67 | 9.463e−11 | 812-846 | PF00992 | Troponin. |
| 1143 | PF00992A | 16.67 | 9.463e−11 | 853-887 | PF00992 | Troponin. |
| 1143 | PF01140D | 15.54 | 4.035e−09 | 797-831 | PF01140 | Matrix protein (MA), |
| | | | | | | p15. |
| 1143 | PF01140D | 15.54 | 6.850e−10 | 811-845 | PF01140 | Matrix protein (MA), |
| | | | | | | p15. |
| 1145 | BL00211A | 12.23 | 2.350e−09 | 122-133 | BL00211 | ABC transporters family |
| | | | | | | proteins. |
| 1145 | BL00617A | 25.53 | 7.000e−10 | 105-147 | BL00617 | RecF protein. |
| 1146 | BL00710 | 12.98 | 3.880e−16 | 182-196 | BL00710 | Phosphoglucomutase and |
| | | | | | | phosphomannomutase |
| | | | | | | phosphoserine signa. |
| 1146 | PR00509A | 14.01 | 1.964e−17 | 181-195 | PR00509 | PHOSPHOGLUCOMUTASE/ |
| | | | | | | PHOSPHOMANNOMUTASE FAMILY |
| | | | | | | SIGNATURE |
| 1146 | PR00509B | 15.05 | 7.353e−19 | 288-307 | PR00509 | PHOSPHOGLUCOMUTASE/ |
| | | | | | | PHOSPHOMANNOMUTASE FAMILY |
| | | | | | | SIGNATURE |
| 1146 | PR00509C | 9.78 | 8.875e−16 | 324-337 | PR00509 | PHOSPHOGLUCOMUTASE/ |
| | | | | | | PHOSPHOMANNOMUTASE FAMILY |
| | | | | | | SIGNATURE |
| 1146 | PR00509D | 9.21 | 3.000e−19 | 352-367 | PR00509 | PHOSPHOGLUCOMUTASE/ |
| | | | | | | PHOSPHOMANNOMUTASE FAMILY |
| | | | | | | SIGNATURE |
| 1147 | BL00415B | 9.91 | 9.206e−09 | 5-40 | BL00415 | Synapsins proteins. |
| 1150 | PR00676D | 10.15 | 9.536e−09 | 47-62 | PR00676 | MASPIN SIGNATURE |
| 1152 | BL01123D | 9.68 | 6.651e−11 | 217-232 | BL01123 | Thermonuclease family |
| | | | | | | proteins. |
| 1154 | BL00226A | 12.77 | 6.500e−16 | 133-147 | BL00226 | Intermediate filaments |
| | | | | | | proteins. |
| 1154 | BL00226B | 23.86 | 1.000e−40 | 229-276 | BL00226 | Intermediate filaments |
| | | | | | | proteins. |
| 1154 | BL00226B | 23.86 | 3.160e−11 | 180-227 | BL00226 | Intermediate filaments |
| | | | | | | proteins. |
| 1154 | BL00226C | 13.23 | 3.700e−25 | 294-324 | BL00226 | Intermediate filaments |
| | | | | | | proteins. |
| 1154 | BL00226D | 19.10 | 1.000e−40 | 395-441 | BL00226 | Intermediate filaments |
| | | | | | | proteins. |
| 1154 | BL01160B | 19.54 | 1.763e−09 | 180-233 | BL01160 | Kinesin light chain |
| | | | | | | repeat proteins. |
| 1154 | BL01312I | 22.76 | 1.758e−09 | 162-210 | BL01312 | Protein secA proteins. |
| 1155 | BL00022B | 7.54 | 1.000e−09 | 1154-1160 | BL00022 | EGF-like domain |
| | | | | | | proteins. |
| 1155 | BL00022B | 7.54 | 3.250e−10 | 1558-1564 | BL00022 | EGF-like domain |
| | | | | | | proteins. |
| 1155 | BL00240B | 24.70 | 8.660e−09 | 3622-3645 | BL00240 | Receptor tyrosine |
| | | | | | | kinase class III |
| | | | | | | proteins. |
| 1155 | BL00243H | 17.53 | 9.645e−09 | 4180-4205 | BL00243 | Integrins beta chain |
| | | | | | | cysteine-rich domain |
| | | | | | | proteins. |
| 1155 | BL00562 | 16.21 | 5.814e−09 | 4127-4157 | BL00562 | Cellulose-binding |
| | | | | | | domain proteins fungal |
| | | | | | | type. |
| 1155 | BL01209 | 9.31 | 2.174e−11 | 370-382 | BL01209 | LDL-receptor class A |
| | | | | | | (LDLRA) domain |
| | | | | | | proteins. |
| 1155 | BL01209 | 9.31 | 4.808e−10 | 414-426 | BL01209 | LDL-receptor class A |
| | | | | | | (LDLRA) domain |
| | | | | | | proteins. |
| 1155 | BL01209 | 9.31 | 6.304e−09 | 245-257 | BL01209 | LDL-receptor class A |
| | | | | | | (LDLRA) domain |
| | | | | | | proteins. |
| 1155 | BL01209 | 9.31 | 9.053e−15 | 330-342 | BL01209 | LDL-receptor class A |
| | | | | | | (LDLRA) domain |
| | | | | | | proteins. |
| 1155 | BL01248 | 11.02 | 3.829e−12 | 1206-1218 | BL01248 | Laminin-type EGF-like |
| | | | | | | (LE) domain proteins. |
| 1155 | BL01248 | 11.02 | 4.724e−13 | 811-823 | BL01248 | Laminin-type EGF-like |
| | | | | | | (LE) domain proteins. |
| 1155 | BL01248 | 11.02 | 8.650e−11 | 1610-1622 | BL01248 | Laminin-type EGF-like |
| | | | | | | (LE) domain proteins. |
| 1155 | DM00060 | 6.92 | 3.700e−10 | 1687-1696 | DM00060 | 338 kw NEUREXIN ALPHA |
| | | | | | | III CYSTEINE. |
| 1155 | DM00060 | 6.92 | 7.000e−11 | 888-897 | DM00060 | 338 kw NEUREXIN ALPHA |
| | | | | | | III CYSTEINE. |
| 1155 | DM00179 | 13.97 | 2.421e−10 | 3386-3395 | DM00179 | w KINASE ALPHA ADHESION |
| | | | | | | T-CELL. |
| 1155 | PD00320A | 14.49 | 3.455e−11 | 1586-1599 | PD00320 | LAMININ CHAIN EGF-LIKE |
| | | | | | | DOMAIN P. |
| 1155 | PD00919B | 9.47 | 8.015e−10 | 1662-1676 | PD00919 | CALCIUM-BINDING |
| | | | | | | PRECURSOR SIGNAL R. |
| 1155 | PD01270A | 17.22 | 3.903e−09 | 431-470 | PD01270 | RECEPTOR FC |
| | | | | | | IMMUNOGLOBULIN AFFIN. |
| 1155 | PD02870B | 18.83 | 3.968e−09 | 2516-2548 | PD02870 | RECEPTOR INTERLEUKIN-1 |
| | | | | | | PRECURSOR. |
| 1155 | PD02870B | 18.83 | 6.300e−10 | 1853-1885 | PD02870 | RECEPTOR INTERLEUKIN-1 |
| | | | | | | PRECURSOR. |
| 1155 | PD02870B | 18.83 | 6.400e−10 | 3198-3230 | PD02870 | RECEPTOR INTERLEUKIN-1 |
| | | | | | | PRECURSOR. |
| 1155 | PD02870B | 18.83 | 6.553e−09 | 2130-2162 | PD02870 | RECEPTOR INTERLEUKIN-1 |
| | | | | | | PRECURSOR. |
| 1155 | PD02870B | 18.83 | 6.649e−09 | 2420-2452 | PD02870 | RECEPTOR INTERLEUKIN-1 |
| | | | | | | PRECURSOR. |
| 1155 | PD02870B | 18.83 | 7.798e−09 | 1759-1791 | PD02870 | RECEPTOR INTERLEUKIN-1 |
| | | | | | | PRECURSOR. |
| 1155 | PD02870B | 18.83 | 9.000e−10 | 2613-2645 | PD02870 | RECEPTOR INTERLEUKIN-1 |
| | | | | | | PRECURSOR. |
| 1155 | PD02870B | 18.83 | 9.809e−09 | 2709-2741 | PD02870 | RECEPTOR INTERLEUKIN-1 |
| | | | | | | PRECURSOR. |
| 1155 | PD02870D | 15.74 | 1.383e−09 | 1853-1887 | PD02870 | RECEPTOR INTERLEUKIN-1 |
| | | | | | | PRECURSOR. |
| 1155 | PR00010A | 11.79 | 1.000e−08 | 4132-4143 | PR00010 | TYPE II EGF-LIKE |
| | | | | | | SIGNATURE |
| 1155 | PR00010C | 11.16 | 9.333e−11 | 3893-3903 | PR00010 | TYPE II EGF-LIKE |
| | | | | | | SIGNATURE |
| 1155 | PR00011A | 14.06 | 9.384e−09 | 1314-1332 | PR00011 | TYPE III EGF-LIKE |
| | | | | | | SIGNATURE |
| 1155 | PR00011B | 13.08 | 6.055e−09 | 1314-1332 | PR00011 | TYPE III EGF-LIKE |
| | | | | | | SIGNATURE |
| 1155 | PR00011C | 24.25 | 8.000e−09 | 920-948 | PR00011 | TYPE III EGF-LIKE |
| | | | | | | SIGNATURE |
| 1155 | PR00011C | 24.25 | 9.518e−11 | 1321-1349 | PR00011 | TYPE III EGF-LIKE |
| | | | | | | SIGNATURE |
| 1155 | PR00011D | 14.03 | 1.000e−11 | 1314-1332 | PR00011 | TYPE III EGF-LIKE |
| | | | | | | SIGNATURE |
| 1155 | PR00011D | 14.03 | 9.169e−10 | 4185-4203 | PR00011 | TYPE III EGF-LIKE |
| | | | | | | SIGNATURE |
| 1155 | PR00261A | 11.02 | 4.296e−12 | 405-426 | PR00261 | LOW DENSITY LIPOPROTEIN |
| | | | | | | (LDL) RECEPTOR |
| | | | | | | SIGNATURE |
| 1155 | PR00261A | 11.02 | 5.895e−17 | 321-342 | PR00261 | LOW DENSITY LIPOPROTEIN |
| | | | | | | (LDL) RECEPTOR |
| | | | | | | SIGNATURE |
| 1155 | PR00261A | 11.02 | 8.676e−13 | 236-257 | PR00261 | LOW DENSITY LIPOPROTEIN |
| | | | | | | (LDL) RECEPTOR |
| | | | | | | SIGNATURE |
| 1155 | PR00261A | 11.02 | 8.984e−15 | 361-382 | PR00261 | LOW DENSITY LIPOPROTEIN |
| | | | | | | (LDL) RECEPTOR |
| | | | | | | SIGNATURE |
| 1155 | PR00261B | 14.12 | 2.356e−12 | 361-382 | PR00261 | LOW DENSITY LIPOPROTEIN |
| | | | | | | (LDL) RECEPTOR |
| | | | | | | SIGNATURE |
| 1155 | PR00261B | 14.12 | 2.588e−14 | 405-426 | PR00261 | LOW DENSITY LIPOPROTEIN |
| | | | | | | (LDL) RECEPTOR |
| | | | | | | SIGNATURE |
| 1155 | PR00261B | 14.12 | 5.500e−17 | 321-342 | PR00261 | LOW DENSITY LIPOPROTEIN |
| | | | | | | (LDL) RECEPTOR |
| | | | | | | SIGNATURE |
| 1155 | PR00261B | 14.12 | 7.662e−11 | 236-257 | PR00261 | LOW DENSITY LIPOPROTEIN |
| | | | | | | (LDL) RECEPTOR |
| | | | | | | SIGNATURE |
| 1155 | PR00261C | 11.37 | 2.826e−13 | 361-382 | PR00261 | LOW DENSITY LIPOPROTEIN |
| | | | | | | (LDL) RECEPTOR |
| | | | | | | SIGNATURE |
| 1155 | PR00261C | 11.37 | 6.373e−14 | 405-426 | PR00261 | LOW DENSITY LIPOPROTEIN |
| | | | | | | (LDL) RECEPTOR |
| | | | | | | SIGNATURE |
| 1155 | PR00261C | 11.37 | 8.500e−20 | 321-342 | PR00261 | LOW DENSITY LIPOPROTEIN |
| | | | | | | (LDL) RECEPTOR |
| | | | | | | SIGNATURE |
| 1155 | PR00261C | 11.37 | 9.014e−12 | 236-257 | PR00261 | LOW DENSITY LIPOPROTEIN |
| | | | | | | (LDL) RECEPTOR |
| | | | | | | SIGNATURE |
| 1155 | PR00261D | 12.47 | 1.305e−16 | 321-342 | PR00261 | LOW DENSITY LIPOPROTEIN |
| | | | | | | (LDL) RECEPTOR |
| | | | | | | SIGNATURE |
| 1155 | PR00261D | 12.47 | 5.000e−15 | 361-382 | PR00261 | LOW DENSITY LIPOPROTEIN |
| | | | | | | (LDL) RECEPTOR |
| | | | | | | SIGNATURE |
| 1155 | PR00261D | 12.47 | 8.734e−14 | 236-257 | PR00261 | LOW DENSITY LIPOPROTEIN |
| | | | | | | (LDL) RECEPTOR |
| | | | | | | SIGNATURE |
| 1155 | PR00261D | 12.47 | 9.859e−14 | 405-426 | PR00261 | LOW DENSITY LIPOPROTEIN |
| | | | | | | (LDL) RECEPTOR |
| | | | | | | SIGNATURE |
| 1155 | PR00261E | 11.08 | 3.727e−12 | 361-382 | PR00261 | LOW DENSITY LIPOPROTEIN |
| | | | | | | (LDL) RECEPTOR |
| | | | | | | SIGNATURE |
| 1155 | PR00261E | 11.08 | 6.906e−13 | 321-342 | PR00261 | LOW DENSITY LIPOPROTEIN |
| | | | | | | (LDL) RECEPTOR |
| | | | | | | SIGNATURE |
| 1155 | PR00261E | 11.08 | 7.171e−11 | 236-257 | PR00261 | LOW DENSITY LIPOPROTEIN |
| | | | | | | (LDL) RECEPTOR |
| | | | | | | SIGNATURE |
| 1155 | PR00261E | 11.08 | 7.545e−12 | 405-426 | PR00261 | LOW DENSITY LIPOPROTEIN |
| | | | | | | (LDL) RECEPTOR |
| | | | | | | SIGNATURE |
| 1155 | PR00261F | 11.57 | 5.355e−14 | 236-257 | PR00261 | LOW DENSITY LIPOPROTEIN |
| | | | | | | (LDL) RECEPTOR |
| | | | | | | SIGNATURE |
| 1155 | PR00261F | 11.57 | 6.280e−10 | 361-382 | PR00261 | LOW DENSITY LIPOPROTEIN |
| | | | | | | (LDL) RECEPTOR |
| | | | | | | SIGNATURE |
| 1155 | PR00261F | 11.57 | 7.589e−16 | 321-342 | PR00261 | LOW DENSITY LIPOPROTEIN |
| | | | | | | (LDL) RECEPTOR |
| | | | | | | SIGNATURE |
| 1155 | PR00261F | 11.57 | 9.603e−12 | 405-426 | PR00261 | LOW DENSITY LIPOPROTEIN |
| | | | | | | (LDL) RECEPTOR |
| | | | | | | SIGNATURE |
| 1155 | PR00764B | 13.56 | 5.091e−10 | 356-376 | PR00764 | COMPLEMENT C9 SIGNATURE |
| 1155 | PR00764B | 13.56 | 5.388e−09 | 316-336 | PR00764 | COMPLEMENT C9 SIGNATURE |
| 1158 | BL00107A | 18.39 | 1.750e−26 | 324-354 | BL00107 | Protein kinases ATP- |
| | | | | | | binding region |
| | | | | | | proteins. |
| 1158 | BL00107B | 13.31 | 5.500e−11 | 389-404 | BL00107 | Protein kinases ATP- |
| | | | | | | binding region |
| | | | | | | proteins. |
| 1158 | BL00479C | 12.01 | 3.571e−11 | 368-380 | BL00479 | Phorbol esters/ |
| | | | | | | diacylglycerol binding |
| | | | | | | domain proteins. |
| 1158 | PF00564B | 24.74 | 8.329e−13 | 275-325 | PF00564 | Octicosapeptide repeat |
| | | | | | | proteins. |
| 1158 | PR00109B | 12.27 | 1.882e−12 | 324-342 | PR00109 | TYROSINE KINASE |
| | | | | | | CATALYTIC DOMAIN |
| | | | | | | SIGNATURE |
| 1163 | PD00247C | 22.11 | 2.125e−09 | 70-97 | PD00247 | POLYPROTEIN COAT |
| | | | | | | PROTEIN TRANSMEMBRANE |
| | | | | | | PRECURSOR. |
| 1163 | PR00049D | 0.00 | 2.786e−10 | 41-55 | PR00049 | WILM'S TUMOUR PROTEIN |
| | | | | | | SIGNATURE |
| 1163 | PR00049D | 0.00 | 4.356e−09 | 42-56 | PR00049 | WILM'S TUMOUR PROTEIN |
| | | | | | | SIGNATURE |
| 1163 | PR00221H | 12.82 | 7.614e−09 | 372-385 | PR00221 | CAULIMOVIRUS COAT |
| | | | | | | PROTEIN SIGNATURE |
| 1164 | BL00649B | 20.68 | 2.966e−10 | 484-529 | BL00649 | G-protein coupled |
| | | | | | | receptors family 2 |
| | | | | | | proteins. |
| 1164 | BL00649C | 17.82 | 5.255e−13 | 542-567 | BL00649 | G-protein coupled |
| | | | | | | receptors family 2 |
| | | | | | | proteins. |
| 1164 | BL00649E | 15.34 | 1.000e−08 | 624-653 | BL00649 | G-protein coupled |
| | | | | | | receptors family 2 |
| | | | | | | proteins. |
| 1164 | BL00649G | 13.52 | 5.208e−09 | 706-731 | BL00649 | G-protein coupled |
| | | | | | | receptors family 2 |
| | | | | | | proteins. |
| 1164 | BL01187A | 9.98 | 1.000e−10 | 137-148 | BL01187 | Calcium-binding EGF- |
| | | | | | | like domain proteins |
| | | | | | | pattern proteins. |
| 1164 | BL01187B | 12.04 | 1.529e−15 | 105-120 | BL01187 | Calcium-binding EGF- |
| | | | | | | like domain proteins |
| | | | | | | pattern proteins. |
| 1164 | BL01187B | 12.04 | 6.250e−17 | 156-171 | BL01187 | Calcium-binding EGF- |
| | | | | | | like domain proteins |
| | | | | | | pattern proteins. |
| 1164 | PR00010C | 11.16 | 7.214e−09 | 110-120 | PR00010 | TYPE II EGF-LIKE |
| | | | | | | SIGNATURE |
| 1164 | PR00249C | 17.08 | 5.708e−10 | 544-567 | PR00249 | SECRETIN-LIKE GPCR |
| | | | | | | SUPERFAMILY SIGNATURE |
| 1164 | PR00249D | 13.09 | 7.429e−09 | 582-607 | PR00249 | SECRETIN-LIKE GPCR |
| | | | | | | SUPERFAMILY SIGNATURE |
| 1165 | BL00216B | 27.64 | 5.000e−34 | 147-196 | BL00216 | Sugar transport |
| | | | | | | proteins. |
| 1165 | PR00171A | 10.00 | 1.429e−11 | 48-58 | PR00171 | SUGAR TRANSPORTER |
| | | | | | | SIGNATURE |
| 1165 | PR00171B | 14.73 | 3.813e−18 | 148-167 | PR00171 | SUGAR TRANSPORTER |
| | | | | | | SIGNATURE |
| 1165 | PR00171C | 10.97 | 3.143e−13 | 307-317 | PR00171 | SUGAR TRANSPORTER |
| | | | | | | SIGNATURE |
| 1165 | PR00171D | 12.76 | 7.750e−20 | 394-415 | PR00171 | SUGAR TRANSPORTER |
| | | | | | | SIGNATURE |
| 1165 | PR00171E | 14.87 | 2.800e−11 | 417-429 | PR00171 | SUGAR TRANSPORTER |
| | | | | | | SIGNATURE |
| 1165 | PR00172A | 9.82 | 5.091e−26 | 297-318 | PR00172 | GLUCOSE TRANSPORTER |
| | | | | | | SIGNATURE |
| 1165 | PR00172B | 8.42 | 9.500e−24 | 332-353 | PR00172 | GLUCOSE TRANSPORTER |
| | | | | | | SIGNATURE |
| 1165 | PR00172C | 9.51 | 5.000e−22 | 363-383 | PR00172 | GLUCOSE TRANSPORTER |
| | | | | | | SIGNATURE |
| 1165 | PR00172D | 9.13 | 6.824e−28 | 394-417 | PR00172 | GLUCOSE TRANSPORTER |
| | | | | | | SIGNATURE |
| 1165 | PR00172E | 8.29 | 2.500e−23 | 427-445 | PR00172 | GLUCOSE TRANSPORTER |
| | | | | | | SIGNATURE |
| 1165 | PR00172F | 8.47 | 7.750e−24 | 457-477 | PR00172 | GLUCOSE TRANSPORTER |
| | | | | | | SIGNATURE |
| 1167 | PD00301A | 10.24 | 1.000e−08 | 277-287 | PD00301 | PROTEIN REPEAT MUSCLE |
| | | | | | | CALCIUM-BI. |
| 1168 | BL00218D | 21.49 | 7.324e−11 | 274-318 | BL00218 | Amino acid permeases |
| | | | | | | proteins. |
| 1168 | BL00218E | 23.30 | 3.475e−09 | 355-394 | BL00218 | Amino acid permeases |
| | | | | | | proteins. |
| 1170 | PD00301A | 10.24 | 6.400e−09 | 73-83 | PD00301 | PROTEIN REPEAT MUSCLE |
| | | | | | | CALCIUM-BI. |
| 1170 | PR00049D | 0.00 | 8.627e−09 | 175-189 | PR00049 | WILM'S TUMOUR PROTEIN |
| | | | | | | SIGNATURE |
| 1172 | BL00453A | 15.57 | 1.000e−16 | 56-70 | BL00453 | FKBP-type peptidyl- |
| | | | | | | prolyl cis-trans |
| | | | | | | isomerase proteins. |
| 1172 | BL00453B | 23.86 | 7.231e−26 | 81-114 | BL00453 | FKBP-type peptidyl- |
| | | | | | | prolyl cis-trans |
| | | | | | | isomerase proteins. |
| 1172 | BL00453C | 9.72 | 4.214e−13 | 122-134 | BL00453 | FKBP-type peptidyl- |
| | | | | | | prolyl cis-trans |
| | | | | | | isomerase proteins. |
| 1176 | BL00136A | 14.35 | 1.720e−11 | 177-189 | BL00136 | Serine proteases, |
| | | | | | | subtilase family, |
| | | | | | | aspartic acid proteins. |
| 1176 | BL00136B | 9.63 | 2.500e−12 | 218-230 | BL00136 | Serine proteases, |
| | | | | | | subtilase family, |
| | | | | | | aspartic acid proteins. |
| 1176 | BL00136C | 8.52 | 9.609e−11 | 392-402 | BL00136 | Serine proteases, |
| | | | | | | subtilase family, |
| | | | | | | aspartic acid proteins. |
| 1176 | PR00723A | 13.37 | 5.200e−17 | 170-189 | PR00723 | SUBTILISIN SERINE |
| | | | | | | PROTEASE FAMILY (S8) |
| | | | | | | SIGNATURE |
| 1176 | PR00723B | 11.22 | 5.050e−13 | 216-229 | PR00723 | SUBTILISIN SERINE |
| | | | | | | PROTEASE FAMILY (S8) |
| | | | | | | SIGNATURE |
| 1176 | PR00723C | 10.64 | 2.929e−13 | 391-407 | PR00723 | SUBTILISIN SERINE |
| | | | | | | PROTEASE FAMILY (S8) |
| | | | | | | SIGNATURE |
| 1182 | BL00420A | 20.42 | 1.138e−09 | 505-533 | BL00420 | Speract receptor repeat |
| | | | | | | proteins domain |
| | | | | | | proteins. |
| 1182 | BL00420A | 20.42 | 1.148e−10 | 1129-1157 | BL00420 | Speract receptor repeat |
| | | | | | | proteins domain |
| | | | | | | proteins. |
| 1182 | BL00420A | 20.42 | 1.173e−12 | 1564-1592 | BL00420 | Speract receptor repeat |
| | | | | | | proteins domain |
| | | | | | | proteins. |
| 1182 | BL00420A | 20.42 | 1.554e−09 | 1069-1097 | BL00420 | Speract receptor repeat |
| | | | | | | proteins domain |
| | | | | | | proteins. |
| 1182 | BL00420A | 20.42 | 1.554e−09 | 928-956 | BL00420 | Speract receptor repeat |
| | | | | | | proteins domain |
| | | | | | | proteins. |
| 1182 | BL00420A | 20.42 | 1.692e−09 | 1507-1535 | BL00420 | Speract receptor repeat |
| | | | | | | proteins domain |
| | | | | | | proteins. |
| 1182 | BL00420A | 20.42 | 1.692e−09 | 1558-1586 | BL00420 | Speract receptor repeat |
| | | | | | | proteins domain |
| | | | | | | proteins. |
| 1182 | BL00420A | 20.42 | 1.738e−10 | 1087-1115 | BL00420 | Speract receptor repeat |
| | | | | | | proteins domain |
| | | | | | | proteins. |
| 1182 | BL00420A | 20.42 | 1.918e−13 | 1378-1406 | BL00420 | Speract receptor repeat |
| | | | | | | proteins domain |
| | | | | | | proteins. |
| 1182 | BL00420A | 20.42 | 1.969e−09 | 1423-1451 | BL00420 | Speract receptor repeat |
| | | | | | | proteins domain |
| | | | | | | proteins. |
| 1182 | BL00420A | 20.42 | 2.328e−10 | 1252-1280 | BL00420 | Speract receptor repeat |
| | | | | | | proteins domain |
| | | | | | | proteins. |
| 1182 | BL00420A | 20.42 | 2.328e−10 | 1297-1325 | BL00420 | Speract receptor repeat |
| | | | | | | proteins domain |
| | | | | | | proteins. |
| 1182 | BL00420A | 20.42 | 2.385e−09 | 655-683 | BL00420 | Speract receptor repeat |
| | | | | | | proteins domain |
| | | | | | | proteins. |
| 1182 | BL00420A | 20.42 | 2.385e−12 | 1294-1322 | BL00420 | Speract receptor repeat |
| | | | | | | proteins domain |
| | | | | | | proteins. |
| 1182 | BL00420A | 20.42 | 2.475e−10 | 1486-1514 | BL00420 | Speract receptor repeat |
| | | | | | | proteins domain |
| | | | | | | proteins. |
| 1182 | BL00420A | 20.42 | 2.523e−09 | 1126-1154 | BL00420 | Speract receptor repeat |
| | | | | | | proteins domain |
| | | | | | | proteins. |
| 1182 | BL00420A | 20.42 | 2.623e−10 | 1057-1085 | BL00420 | Speract receptor repeat |
| | | | | | | proteins domain |
| | | | | | | proteins. |
| 1182 | BL00420A | 20.42 | 2.662e−09 | 673-701 | BL00420 | Speract receptor repeat |
| | | | | | | proteins domain |
| | | | | | | proteins. |
| 1182 | BL00420A | 20.42 | 2.918e−10 | 754-782 | BL00420 | Speract receptor repeat |
| | | | | | | proteins domain |
| | | | | | | proteins. |
| 1182 | BL00420A | 20.42 | 3.213e−10 | 1420-1448 | BL00420 | Speract receptor repeat |
| | | | | | | proteins domain |
| | | | | | | proteins. |
| 1182 | BL00420A | 20.42 | 3.492e−09 | 1009-1037 | BL00420 | Speract receptor repeat |
| | | | | | | proteins domain |
| | | | | | | proteins. |
| 1182 | BL00420A | 20.42 | 3.631e−09 | 670-698 | BL00420 | Speract receptor repeat |
| | | | | | | proteins domain |
| | | | | | | proteins. |
| 1182 | BL00420A | 20.42 | 3.769e−09 | 1459-1487 | BL00420 | Speract receptor repeat |
| | | | | | | proteins domain |
| | | | | | | proteins. |
| 1182 | BL00420A | 20.42 | 3.945e−11 | 922-950 | BL00420 | Speract receptor repeat |
| | | | | | | proteins domain |
| | | | | | | proteins. |
| 1182 | BL00420A | 20.42 | 3.951e−10 | 1579-1607 | BL00420 | Speract receptor repeat |
| | | | | | | proteins domain |
| | | | | | | proteins. |
| 1182 | BL00420A | 20.42 | 4.046e−09 | 1339-1367 | BL00420 | Speract receptor repeat |
| | | | | | | proteins domain |
| | | | | | | proteins. |
| 1182 | BL00420A | 20.42 | 4.246e−10 | 1555-1583 | BL00420 | Speract receptor repeat |
| | | | | | | proteins domain |
| | | | | | | proteins. |
| 1182 | BL00420A | 20.42 | 4.635e−12 | 787-815 | BL00420 | Speract receptor repeat |
| | | | | | | proteins domain |
| | | | | | | proteins. |
| 1182 | BL00420A | 20.42 | 4.689e−10 | 871-899 | BL00420 | Speract receptor repeat |
| | | | | | | proteins domain |
| | | | | | | proteins. |
| 1182 | BL00420A | 20.42 | 4.836e−10 | 820-848 | BL00420 | Speract receptor repeat |
| | | | | | | proteins domain |
| | | | | | | proteins. |
| 1182 | BL00420A | 20.42 | 4.984e−10 | 1489-1517 | BL00420 | Speract receptor repeat |
| | | | | | | proteins domain |
| | | | | | | proteins. |
| 1182 | BL00420A | 20.42 | 4.984e−10 | 796-824 | BL00420 | Speract receptor repeat |
| | | | | | | proteins domain |
| | | | | | | proteins. |
| 1182 | BL00420A | 20.42 | 5.154e−12 | 817-845 | BL00420 | Speract receptor repeat |
| | | | | | | proteins domain |
| | | | | | | proteins. |
| 1182 | BL00420A | 20.42 | 5.292e−09 | 838-866 | BL00420 | Speract receptor repeat |
| | | | | | | proteins domain |
| | | | | | | proteins. |
| 1182 | BL00420A | 20.42 | 5.431e−09 | 1183-1211 | BL00420 | Speract receptor repeat |
| | | | | | | proteins domain |
| | | | | | | proteins. |
| 1182 | BL00420A | 20.42 | 5.569e−09 | 1093-1121 | BL00420 | Speract receptor repeat |
| | | | | | | proteins domain |
| | | | | | | proteins. |
| 1182 | BL00420A | 20.42 | 5.574e−10 | 724-752 | BL00420 | Speract receptor repeat |
| | | | | | | proteins domain |
| | | | | | | proteins. |
| 1182 | BL00420A | 20.42 | 5.574e−10 | 751-779 | BL00420 | Speract receptor repeat |
| | | | | | | proteins domain |
| | | | | | | proteins. |
| 1182 | BL00420A | 20.42 | 5.721e−10 | 1567-1595 | BL00420 | Speract receptor repeat |
| | | | | | | proteins domain |
| | | | | | | proteins. |
| 1182 | BL00420A | 20.42 | 5.745e−11 | 994-1022 | BL00420 | Speract receptor repeat |
| | | | | | | proteins domain |
| | | | | | | proteins. |
| 1182 | BL00420A | 20.42 | 5.846e−12 | 991-1019 | BL00420 | Speract receptor repeat |
| | | | | | | proteins domain |
| | | | | | | proteins. |
| 1182 | BL00420A | 20.42 | 5.869e−10 | 1372-1400 | BL00420 | Speract receptor repeat |
| | | | | | | proteins domain |
| | | | | | | proteins. |
| 1182 | BL00420A | 20.42 | 5.985e−09 | 1012-1040 | BL00420 | Speract receptor repeat |
| | | | | | | proteins domain |
| | | | | | | proteins. |
| 1182 | BL00420A | 20.42 | 6.459e−10 | 1045-1073 | BL00420 | Speract receptor repeat |
| | | | | | | proteins domain |
| | | | | | | proteins. |
| 1182 | BL00420A | 20.42 | 6.607e−10 | 805-833 | BL00420 | Speract receptor repeat |
| | | | | | | proteins domain |
| | | | | | | proteins. |
| 1182 | BL00420A | 20.42 | 6.677e−09 | 958-986 | BL00420 | Speract receptor repeat |
| | | | | | | proteins domain |
| | | | | | | proteins. |
| 1182 | BL00420A | 20.42 | 6.815e−09 | 1369-1397 | BL00420 | Speract receptor repeat |
| | | | | | | proteins domain |
| | | | | | | proteins. |
| 1182 | BL00420A | 20.42 | 6.815e−09 | 1471-1499 | BL00420 | Speract receptor repeat |
| | | | | | | proteins domain |
| | | | | | | proteins. |
| 1182 | BL00420A | 20.42 | 6.815e−09 | 895-923 | BL00420 | Speract receptor repeat |
| | | | | | | proteins domain |
| | | | | | | proteins. |
| 1182 | BL00420A | 20.42 | 6.885e−12 | 1321-1349 | BL00420 | Speract receptor repeat |
| | | | | | | proteins domain |
| | | | | | | proteins. |
| 1182 | BL00420A | 20.42 | 6.954e−09 | 1483-1511 | BL00420 | Speract receptor repeat |
| | | | | | | proteins domain |
| | | | | | | proteins. |
| 1182 | BL00420A | 20.42 | 7.049e−10 | 1180-1208 | BL00420 | Speract receptor repeat |
| | | | | | | proteins domain |
| | | | | | | proteins. |
| 1182 | BL00420A | 20.42 | 7.049e−10 | 847-875 | BL00420 | Speract receptor repeat |
| | | | | | | proteins domain |
| | | | | | | proteins. |
| 1182 | BL00420A | 20.42 | 7.055e−11 | 1504-1532 | BL00420 | Speract receptor repeat |
| | | | | | | proteins domain |
| | | | | | | proteins. |
| 1182 | BL00420A | 20.42 | 7.218e−11 | 1315-1343 | BL00420 | Speract receptor repeat |
| | | | | | | proteins domain |
| | | | | | | proteins. |
| 1182 | BL00420A | 20.42 | 7.231e−09 | 1285-1313 | BL00420 | Speract receptor repeat |
| | | | | | | proteins domain |
| | | | | | | proteins. |
| 1182 | BL00420A | 20.42 | 7.231e−09 | 850-878 | BL00420 | Speract receptor repeat |
| | | | | | | proteins domain |
| | | | | | | proteins. |
| 1182 | BL00420A | 20.42 | 7.245e−13 | 1099-1127 | BL00420 | Speract receptor repeat |
| | | | | | | proteins domain |
| | | | | | | proteins. |
| 1182 | BL00420A | 20.42 | 7.369e−09 | 1162-1190 | BL00420 | Speract receptor repeat |
| | | | | | | proteins domain |
| | | | | | | proteins. |
| 1182 | BL00420A | 20.42 | 7.369e−09 | 634-662 | BL00420 | Speract receptor repeat |
| | | | | | | proteins domain |
| | | | | | | proteins. |
| 1182 | BL00420A | 20.42 | 7.429e−13 | 1249-1277 | BL00420 | Speract receptor repeat |
| | | | | | | proteins domain |
| | | | | | | proteins. |
| 1182 | BL00420A | 20.42 | 7.492e−10 | 721-749 | BL00420 | Speract receptor repeat |
| | | | | | | proteins domain |
| | | | | | | proteins. |
| 1182 | BL00420A | 20.42 | 7.785e−09 | 1048-1076 | BL00420 | Speract receptor repeat |
| | | | | | | proteins domain |
| | | | | | | proteins. |
| 1182 | BL00420A | 20.42 | 7.785e−09 | 748-776 | BL00420 | Speract receptor repeat |
| | | | | | | proteins domain |
| | | | | | | proteins. |
| 1182 | BL00420A | 20.42 | 7.934e−10 | 904-932 | BL00420 | Speract receptor repeat |
| | | | | | | proteins domain |
| | | | | | | proteins. |
| 1182 | BL00420A | 20.42 | 8.062e−09 | 1171-1199 | BL00420 | Speract receptor repeat |
| | | | | | | proteins domain |
| | | | | | | proteins. |
| 1182 | BL00420A | 20.42 | 8.338e−09 | 1522-1550 | BL00420 | Speract receptor repeat |
| | | | | | | proteins domain |
| | | | | | | proteins. |
| 1182 | BL00420A | 20.42 | 8.377e−10 | 1132-1160 | BL00420 | Speract receptor repeat |
| | | | | | | proteins domain |
| | | | | | | proteins. |
| 1182 | BL00420A | 20.42 | 8.615e−09 | 1117-1145 | BL00420 | Speract receptor repeat |
| | | | | | | proteins domain |
| | | | | | | proteins. |
| 1182 | BL00420A | 20.42 | 8.615e−09 | 1306-1334 | BL00420 | Speract receptor repeat |
| | | | | | | proteins domain |
| | | | | | | proteins. |
| 1182 | BL00420A | 20.42 | 8.672e−10 | 1270-1298 | BL00420 | Speract receptor repeat |
| | | | | | | proteins domain |
| | | | | | | proteins. |
| 1182 | BL00420A | 20.42 | 8.691e−11 | 1375-1403 | BL00420 | Speract receptor repeat |
| | | | | | | proteins domain |
| | | | | | | proteins. |
| 1182 | BL00420A | 20.42 | 8.754e−09 | 529-557 | BL00420 | Speract receptor repeat |
| | | | | | | proteins domain |
| | | | | | | proteins. |
| 1182 | BL00420A | 20.42 | 8.967e−10 | 1492-1520 | BL00420 | Speract receptor repeat |
| | | | | | | proteins domain |
| | | | | | | proteins. |
| 1182 | BL00420A | 20.42 | 9.031e−09 | 1156-1184 | BL00420 | Speract receptor repeat |
| | | | | | | proteins domain |
| | | | | | | proteins. |
| 1182 | BL00420A | 20.42 | 9.031e−09 | 931-959 | BL00420 | Speract receptor repeat |
| | | | | | | proteins domain |
| | | | | | | proteins. |
| 1182 | BL00420A | 20.42 | 9.115e−10 | 781-809 | BL00420 | Speract receptor repeat |
| | | | | | | proteins domain |
| | | | | | | proteins. |
| 1182 | BL00420A | 20.42 | 9.115e−10 | 910-938 | BL00420 | Speract receptor repeat |
| | | | | | | proteins domain |
| | | | | | | proteins. |
| 1182 | BL00420A | 20.42 | 9.169e−09 | 925-953 | BL00420 | Speract receptor repeat |
| | | | | | | proteins domain |
| | | | | | | proteins. |
| 1182 | BL00420A | 20.42 | 9.182e−11 | 1414-1442 | BL00420 | Speract receptor repeat |
| | | | | | | proteins domain |
| | | | | | | proteins. |
| 1182 | BL00420A | 20.42 | 9.262e−10 | 1411-1439 | BL00420 | Speract receptor repeat |
| | | | | | | proteins domain |
| | | | | | | proteins. |
| 1182 | BL00420A | 20.42 | 9.446e−09 | 1402-1430 | BL00420 | Speract receptor repeat |
| | | | | | | proteins domain |
| | | | | | | proteins. |
| 1182 | BL00420A | 20.42 | 9.449e−13 | 1318-1346 | BL00420 | Speract receptor repeat |
| | | | | | | proteins domain |
| | | | | | | proteins. |
| 1182 | BL00420A | 20.42 | 9.585e−09 | 1123-1151 | BL00420 | Speract receptor repeat |
| | | | | | | proteins domain |
| | | | | | | proteins. |
| 1182 | BL00420A | 20.42 | 9.585e−09 | 637-665 | BL00420 | Speract receptor repeat |
| | | | | | | proteins domain |
| | | | | | | proteins. |
| 1182 | BL00420A | 20.42 | 9.705e−10 | 919-947 | BL00420 | Speract receptor repeat |
| | | | | | | proteins domain |
| | | | | | | proteins. |
| 1182 | BL00420A | 20.42 | 9.723e−09 | 1477-1505 | BL00420 | Speract receptor repeat |
| | | | | | | proteins domain |
| | | | | | | proteins. |
| 1182 | BL00420A | 20.42 | 9.852e−10 | 526-554 | BL00420 | Speract receptor repeat |
| | | | | | | proteins domain |
| | | | | | | proteins. |
| 1182 | BL00420A | 20.42 | 9.862e−09 | 649-677 | BL00420 | Speract receptor repeat |
| | | | | | | proteins domain |
| | | | | | | proteins. |
| 1182 | BL00604F | 5.96 | 7.020e−09 | 1433-1477 | BL00604 | Synaptophysin/ |
| | | | | | | synaptoporin proteins. |
| 1182 | BL01113A | 17.99 | 1.000e−09 | 1123-1149 | BL01113 | c1q domain proteins. |
| 1182 | BL01113A | 17.99 | 1.000e−09 | 1405-1431 | BL01113 | c1q domain proteins. |
| 1182 | BL01113A | 17.99 | 1.000e−10 | 1396-1422 | BL01113 | C1q domain proteins. |
| 1182 | BL01113A | 17.99 | 1.000e−10 | 514-540 | BL01113 | c1q domain proteins. |
| 1182 | BL01113A | 17.99 | 1.191e−10 | 730-756 | BL01113 | c1q domain proteins. |
| 1182 | BL01113A | 17.99 | 1.231e−12 | 1492-1518 | BL01113 | c1q domain proteins. |
| 1182 | BL01113A | 17.99 | 1.243e−13 | 1177-1203 | BL01113 | c1q domain proteins. |
| 1182 | BL01113A | 17.99 | 1.519e−09 | 886-912 | BL01113 | c1q domain proteins. |
| 1182 | BL01113A | 17.99 | 1.545e−14 | 1138-1164 | BL01113 | c1q domain proteins. |
| 1182 | BL01113A | 17.99 | 1.614e−11 | 640-666 | BL01113 | c1q domain proteins. |
| 1182 | BL01113A | 17.99 | 1.614e−11 | 805-831 | BL01113 | c1q domain proteins. |
| 1182 | BL01113A | 17.99 | 1.614e−11 | 985-1011 | BL01113 | c1q domain proteins. |
| 1182 | BL01113A | 17.99 | 1.818e−11 | 751-777 | BL01113 | c1q domain proteins. |
| 1182 | BL01113A | 17.99 | 1.865e−09 | 523-549 | BL01113 | c1q domain proteins. |
| 1182 | BL01113A | 17.99 | 1.865e−09 | 979-1005 | BL01113 | c1q domain proteins. |
| 1182 | BL01113A | 17.99 | 1.923e−12 | 1387-1413 | BL01113 | c1q domain proteins. |
| 1182 | BL01113A | 17.99 | 1.957e−10 | 535-561 | BL01113 | c1q domain proteins. |
| 1182 | BL01113A | 17.99 | 1.957e−10 | 655-681 | BL01113 | c1q domain proteins. |
| 1182 | BL01113A | 17.99 | 2.023e−11 | 715-741 | BL01113 | c1q domain proteins. |
| 1182 | BL01113A | 17.99 | 2.038e−09 | 718-744 | BL01113 | c1q domain proteins. |
| 1182 | BL01113A | 17.99 | 2.216e−13 | 1372-1398 | BL01113 | c1q domain proteins. |
| 1182 | BL01113A | 17.99 | 2.385e−09 | 1450-1476 | BL01113 | c1q domain proteins. |
| 1182 | BL01113A | 17.99 | 2.452e−15 | 673-699 | BL01113 | c1q domain proteins. |
| 1182 | BL01113A | 17.99 | 2.459e−13 | 1225-1251 | BL01113 | c1q domain proteins. |
| 1182 | BL01113A | 17.99 | 2.532e−10 | 1084-1110 | BL01113 | c1q domain proteins. |
| 1182 | BL01113A | 17.99 | 2.532e−10 | 1468-1494 | BL01113 | c1q domain proteins. |
| 1182 | BL01113A | 17.99 | 2.532e−10 | 754-780 | BL01113 | c1q domain proteins. |
| 1182 | BL01113A | 17.99 | 2.558e−09 | 712-738 | BL01113 | c1q domain proteins. |
| 1182 | BL01113A | 17.99 | 2.558e−09 | 811-837 | BL01113 | c1q domain proteins. |
| 1182 | BL01113A | 17.99 | 2.636e−11 | 1048-1074 | BL01113 | c1q domain proteins. |
| 1182 | BL01113A | 17.99 | 2.723e−10 | 1561-1587 | BL01113 | c1q domain proteins. |
| 1182 | BL01113A | 17.99 | 2.731e−09 | 1108-1134 | BL01113 | c1q domain proteins. |
| 1182 | BL01113A | 17.99 | 2.915e−10 | 1081-1107 | BL01113 | c1q domain proteins. |
| 1182 | BL01113A | 17.99 | 3.045e−11 | 727-753 | BL01113 | c1q domain proteins. |
| 1182 | BL01113A | 17.99 | 3.298e−10 | 1585-1611 | BL01113 | c1q domain proteins. |
| 1182 | BL01113A | 17.99 | 3.298e−10 | 1594-1620 | BL01113 | c1q domain proteins. |
| 1182 | BL01113A | 17.99 | 3.432e−13 | 787-813 | BL01113 | c1q domain proteins. |
| 1182 | BL01113A | 17.99 | 3.455e−11 | 922-948 | BL01113 | c1q domain proteins. |
| 1182 | BL01113A | 17.99 | 3.455e−14 | 1555-1581 | BL01113 | c1q domain proteins. |
| 1182 | BL01113A | 17.99 | 3.489e−10 | 688-714 | BL01113 | c1q domain proteins. |
| 1182 | BL01113A | 17.99 | 3.596e−09 | 1411-1437 | BL01113 | c1q domain proteins. |
| 1182 | BL01113A | 17.99 | 3.596e−09 | 685-711 | BL01113 | c1q domain proteins. |
| 1182 | BL01113A | 17.99 | 3.596e−09 | 739-765 | BL01113 | c1q domain proteins. |
| 1182 | BL01113A | 17.99 | 3.769e−09 | 1117-1143 | BL01113 | c1q domain proteins. |
| 1182 | BL01113A | 17.99 | 3.769e−09 | 517-543 | BL01113 | C1q domain proteins. |
| 1182 | BL01113A | 17.99 | 3.769e−09 | 736-762 | BL01113 | C1q domain proteins. |
| 1182 | BL01113A | 17.99 | 3.872e−10 | 724-750 | BL01113 | C1q domain proteins. |
| 1182 | BL01113A | 17.99 | 3.942e−09 | 1384-1410 | BL01113 | C1q domain proteins. |
| 1182 | BL01113A | 17.99 | 3.942e−09 | 1414-1440 | BL01113 | C1q domain proteins. |
| 1182 | BL01113A | 17.99 | 4.064e−10 | 1051-1077 | BL01113 | C1q domain proteins. |
| 1182 | BL01113A | 17.99 | 4.068e−11 | 931-957 | BL01113 | C1q domain proteins. |
| 1182 | BL01113A | 17.99 | 4.255e−10 | 1375-1401 | BL01113 | C1q domain proteins. |
| 1182 | BL01113A | 17.99 | 4.288e−09 | 625-651 | BL01113 | C1q domain proteins. |
| 1182 | BL01113A | 17.99 | 4.447e−10 | 1480-1506 | BL01113 | C1q domain proteins. |
| 1182 | BL01113A | 17.99 | 4.447e−10 | 1570-1596 | BL01113 | C1q domain proteins. |
| 1182 | BL01113A | 17.99 | 4.462e−12 | 1000-1026 | BL01113 | C1q domain proteins. |
| 1182 | BL01113A | 17.99 | 4.462e−12 | 1249-1275 | BL01113 | C1q domain proteins. |
| 1182 | BL01113A | 17.99 | 4.462e−12 | 895-921 | BL01113 | C1q domain proteins. |
| 1182 | BL01113A | 17.99 | 4.477e−11 | 1318-1344 | BL01113 | C1q domain proteins. |
| 1182 | BL01113A | 17.99 | 4.635e−09 | 1027-1053 | BL01113 | C1q domain proteins. |
| 1182 | BL01113A | 17.99 | 4.635e−09 | 1255-1281 | BL01113 | C1q domain proteins. |
| 1182 | BL01113A | 17.99 | 4.635e−09 | 601-627 | BL01113 | C1q domain proteins. |
| 1182 | BL01113A | 17.99 | 4.808e−09 | 1279-1305 | BL01113 | C1q domain proteins. |
| 1182 | BL01113A | 17.99 | 4.830e−10 | 1057-1083 | BL01113 | C1q domain proteins. |
| 1182 | BL01113A | 17.99 | 4.830e−10 | 1312-1338 | BL01113 | C1q domain proteins. |
| 1182 | BL01113A | 17.99 | 4.830e−10 | 595-621 | BL01113 | C1q domain proteins. |
| 1182 | BL01113A | 17.99 | 4.830e−10 | 742-768 | BL01113 | C1q domain proteins. |
| 1182 | BL01113A | 17.99 | 4.886e−11 | 1267-1293 | BL01113 | C1q domain proteins. |
| 1182 | BL01113A | 17.99 | 4.892e−13 | 796-822 | BL01113 | C1q domain proteins. |
| 1182 | BL01113A | 17.99 | 4.923e−12 | 814-840 | BL01113 | C1q domain proteins. |
| 1182 | BL01113A | 17.99 | 4.981e−09 | 1213-1239 | BL01113 | C1q domain proteins. |
| 1182 | BL01113A | 17.99 | 5.021e−10 | 907-933 | BL01113 | C1q domain proteins. |
| 1182 | BL01113A | 17.99 | 5.091e−11 | 1489-1515 | BL01113 | C1q domain proteins. |
| 1182 | BL01113A | 17.99 | 5.154e−09 | 1321-1347 | BL01113 | C1q domain proteins. |
| 1182 | BL01113A | 17.99 | 5.154e−09 | 1429-1455 | BL01113 | C1q domain proteins. |
| 1182 | BL01113A | 17.99 | 5.295e−11 | 1045-1071 | BL01113 | C1q domain proteins. |
| 1182 | BL01113A | 17.99 | 5.295e−11 | 1369-1395 | BL01113 | C1q domain proteins. |
| 1182 | BL01113A | 17.99 | 5.295e−11 | 1558-1584 | BL01113 | C1q domain proteins. |
| 1182 | BL01113A | 17.99 | 5.378e−13 | 1303-1329 | BL01113 | C1q domain proteins. |
| 1182 | BL01113A | 17.99 | 5.378e−13 | 1357-1383 | BL01113 | C1q domain proteins. |
| 1182 | BL01113A | 17.99 | 5.385e−12 | 1354-1380 | BL01113 | C1q domain proteins. |
| 1182 | BL01113A | 17.99 | 5.385e−12 | 529-555 | BL01113 | C1q domain proteins. |
| 1182 | BL01113A | 17.99 | 5.404e−10 | 1063-1089 | BL01113 | C1q domain proteins. |
| 1182 | BL01113A | 17.99 | 5.500e−09 | 1135-1161 | BL01113 | C1q domain proteins. |
| 1182 | BL01113A | 17.99 | 5.500e−09 | 898-924 | BL01113 | C1q domain proteins. |
| 1182 | BL01113A | 17.99 | 5.615e−12 | 1477-1503 | BL01113 | C1q domain proteins. |
| 1182 | BL01113A | 17.99 | 5.622e−13 | 766-792 | BL01113 | C1q domain proteins. |
| 1182 | BL01113A | 17.99 | 5.636e−14 | 1564-1590 | BL01113 | C1q domain proteins. |
| 1182 | BL01113A | 17.99 | 5.705e−11 | 781-807 | BL01113 | C1q domain proteins. |
| 1182 | BL01113A | 17.99 | 5.787e−10 | 604-630 | BL01113 | C1q domain proteins. |
| 1182 | BL01113A | 17.99 | 5.846e−09 | 1285-1311 | BL01113 | C1q domain proteins. |
| 1182 | BL01113A | 17.99 | 6.019e−09 | 823-849 | BL01113 | C1q domain proteins. |
| 1182 | BL01113A | 17.99 | 6.114e−11 | 1348-1374 | BL01113 | C1q domain proteins. |
| 1182 | BL01113A | 17.99 | 6.182e−14 | 1495-1521 | BL01113 | C1q domain proteins. |
| 1182 | BL01113A | 17.99 | 6.192e−09 | 1402-1428 | BL01113 | C1q domain proteins. |
| 1182 | BL01113A | 17.99 | 6.365e−09 | 904-930 | BL01113 | C1q domain proteins. |
| 1182 | BL01113A | 17.99 | 6.455e−14 | 994-1020 | BL01113 | C1q domain proteins. |
| 1182 | BL01113A | 17.99 | 6.538e−09 | 1186-1212 | BL01113 | C1q domain proteins. |
| 1182 | BL01113A | 17.99 | 6.538e−09 | 772-798 | BL01113 | C1q domain proteins. |
| 1182 | BL01113A | 17.99 | 6.553e−10 | 1453-1479 | BL01113 | C1q domain proteins. |
| 1182 | BL01113A | 17.99 | 6.553e−10 | 709-735 | BL01113 | C1q domain proteins. |
| 1182 | BL01113A | 17.99 | 6.595e−13 | 721-747 | BL01113 | C1q domain proteins. |
| 1182 | BL01113A | 17.99 | 6.712e−09 | 1501-1527 | BL01113 | C1q domain proteins. |
| 1182 | BL01113A | 17.99 | 6.727e−11 | 508-534 | BL01113 | C1q domain proteins. |
| 1182 | BL01113A | 17.99 | 6.769e−12 | 790-816 | BL01113 | C1q domain proteins. |
| 1182 | BL01113A | 17.99 | 7.000e−12 | 1129-1155 | BL01113 | C1q domain proteins. |
| 1182 | BL01113A | 17.99 | 7.058e−09 | 1519-1545 | BL01113 | C1q domain proteins. |
| 1182 | BL01113A | 17.99 | 7.058e−09 | 901-927 | BL01113 | C1q domain proteins. |
| 1182 | BL01113A | 17.99 | 7.058e−09 | 910-936 | BL01113 | C1q domain proteins. |
| 1182 | BL01113A | 17.99 | 7.081e−13 | 682-708 | BL01113 | C1q domain proteins. |
| 1182 | BL01113A | 17.99 | 7.128e−10 | 1327-1353 | BL01113 | C1q domain proteins. |
| 1182 | BL01113A | 17.99 | 7.136e−11 | 1423-1449 | BL01113 | C1q domain proteins. |
| 1182 | BL01113A | 17.99 | 7.231e−09 | 619-645 | BL01113 | C1q domain proteins. |
| 1182 | BL01113A | 17.99 | 7.231e−09 | 703-729 | BL01113 | C1q domain proteins. |
| 1182 | BL01113A | 17.99 | 7.273e−14 | 1297-1323 | BL01113 | C1q domain proteins. |
| 1182 | BL01113A | 17.99 | 7.341e−11 | 1258-1284 | BL01113 | C1q domain proteins. |
| 1182 | BL01113A | 17.99 | 7.341e−11 | 610-636 | BL01113 | C1q domain proteins. |
| 1182 | BL01113A | 17.99 | 7.387e−15 | 1363-1389 | BL01113 | C1q domain proteins. |
| 1182 | BL01113A | 17.99 | 7.511e−10 | 1054-1080 | BL01113 | C1q domain proteins. |
| 1182 | BL01113A | 17.99 | 7.511e−10 | 1075-1101 | BL01113 | C1q domain proteins. |
| 1182 | BL01113A | 17.99 | 7.545e−11 | 1420-1446 | BL01113 | C1q domain proteins. |
| 1182 | BL01113A | 17.99 | 7.545e−11 | 1459-1485 | BL01113 | C1q domain proteins. |
| 1182 | BL01113A | 17.99 | 7.545e−11 | 784-810 | BL01113 | C1q domain proteins. |
| 1182 | BL01113A | 17.99 | 7.545e−14 | 1114-1140 | BL01113 | C1q domain proteins. |
| 1182 | BL01113A | 17.99 | 7.568e−13 | 1381-1407 | BL01113 | C1q domain proteins. |
| 1182 | BL01113A | 17.99 | 7.577e−09 | 1210-1236 | BL01113 | C1q domain proteins. |
| 1182 | BL01113A | 17.99 | 7.577e−09 | 1336-1362 | BL01113 | C1q domain proteins. |
| 1182 | BL01113A | 17.99 | 7.577e−09 | 631-657 | BL01113 | C1q domain proteins. |
| 1182 | BL01113A | 17.99 | 7.750e−09 | 1183-1209 | BL01113 | C1q domain proteins. |
| 1182 | BL01113A | 17.99 | 7.750e−09 | 1252-1278 | BL01113 | C1q domain proteins. |
| 1182 | BL01113A | 17.99 | 7.923e−09 | 1102-1128 | BL01113 | C1q domain proteins. |
| 1182 | BL01113A | 17.99 | 7.923e−09 | 1513-1539 | BL01113 | C1q domain proteins. |
| 1182 | BL01113A | 17.99 | 7.923e−12 | 880-906 | BL01113 | C1q domain proteins. |
| 1182 | BL01113A | 17.99 | 8.085e−10 | 1465-1491 | BL01113 | C1q domain proteins. |
| 1182 | BL01113A | 17.99 | 8.096e−09 | 1306-1332 | BL01113 | C1q domain proteins. |
| 1182 | BL01113A | 17.99 | 8.138e−16 | 1486-1512 | BL01113 | C1q domain proteins. |
| 1182 | BL01113A | 17.99 | 8.154e−12 | 1147-1173 | BL01113 | C1q domain proteins. |
| 1182 | BL01113A | 17.99 | 8.269e−09 | 958-984 | BL01113 | C1q domain proteins. |
| 1182 | BL01113A | 17.99 | 8.277e−10 | 1417-1443 | BL01113 | C1q domain proteins. |
| 1182 | BL01113A | 17.99 | 8.364e−11 | 1567-1593 | BL01113 | C1q domain proteins. |
| 1182 | BL01113A | 17.99 | 8.468e−10 | 532-558 | BL01113 | C1q domain proteins. |
| 1182 | BL01113A | 17.99 | 8.615e−12 | 1132-1158 | BL01113 | C1q domain proteins. |
| 1182 | BL01113A | 17.99 | 8.636e−14 | 1288-1314 | BL01113 | C1q domain proteins. |
| 1182 | BL01113A | 17.99 | 8.660e−10 | 889-915 | BL01113 | C1q domain proteins. |
| 1182 | BL01113A | 17.99 | 8.788e−09 | 691-717 | BL01113 | C1q domain proteins. |
| 1182 | BL01113A | 17.99 | 8.788e−09 | 760-786 | BL01113 | C1q domain proteins. |
| 1182 | BL01113A | 17.99 | 8.846e−12 | 1099-1125 | BL01113 | C1q domain proteins. |
| 1182 | BL01113A | 17.99 | 8.977e−11 | 1072-1098 | BL01113 | C1q domain proteins. |
| 1182 | BL01113A | 17.99 | 9.043e−10 | 1573-1599 | BL01113 | C1q domain proteins. |
| 1182 | BL01113A | 17.99 | 9.129e−15 | 1378-1404 | BL01113 | C1q domain proteins. |
| 1182 | BL01113A | 17.99 | 9.135e−09 | 1462-1488 | BL01113 | C1q domain proteins. |
| 1182 | BL01113A | 17.99 | 9.135e−09 | 859-885 | BL01113 | C1q domain proteins. |
| 1182 | BL01113A | 17.99 | 9.234e−10 | 1216-1242 | BL01113 | C1q domain proteins. |
| 1182 | BL01113A | 17.99 | 9.270e−13 | 775-801 | BL01113 | C1q domain proteins. |
| 1182 | BL01113A | 17.99 | 9.270e−13 | 991-1017 | BL01113 | C1q domain proteins. |
| 1182 | BL01113A | 17.99 | 9.426e−10 | 1066-1092 | BL01113 | C1q domain proteins. |
| 1182 | BL01113A | 17.99 | 9.426e−10 | 799-825 | BL01113 | C1q domain proteins. |
| 1182 | BL01113A | 17.99 | 9.769e−12 | 1003-1029 | BL01113 | C1q domain proteins. |
| 1182 | BL01113A | 17.99 | 9.769e−12 | 505-531 | BL01113 | C1q domain proteins. |
| 1182 | BL01113A | 17.99 | 9.827e−09 | 1180-1206 | BL01113 | C1q domain proteins. |
| 1182 | BL01113A | 17.99 | 9.827e−09 | 1588-1614 | BL01113 | C1q domain proteins. |
| 1182 | BL01113A | 17.99 | 9.827e−09 | 955-981 | BL01113 | C1q domain proteins. |
| 1182 | DM01418A | 20.83 | 1.000e−40 | 1668-1715 | DM01418 | 352 FIBRILLAR COLLAGEN |
| | | | | | | CARBOXYL-TERMINAL. |
| 1182 | DM01418B | 22.51 | 5.821e−39 | 1763-1804 | DM01418 | 352 FIBRILLAR COLLAGEN |
| | | | | | | CARBOXYL-TERMINAL. |
| 1182 | DM01418C | 20.48 | 1.000e−34 | 1831-1872 | DM01418 | 352 FIBRILLAR COLLAGEN |
| | | | | | | CARBOXYL-TERMINAL. |
| 1185 | BL00514C | 17.41 | 9.082e−28 | 264-300 | BL00514 | Fibrinogen beta and |
| | | | | | | gamma chains C-terminal |
| | | | | | | domain proteins. |
| 1185 | BL00514D | 15.35 | 3.118e−11 | 305-317 | BL00514 | Fibrinogen beta and |
| | | | | | | gamma chains C-terminal |
| | | | | | | domain proteins. |
| 1185 | BL00514G | 15.98 | 1.000e−14 | 391-420 | BL00514 | Fibrinogen beta and |
| | | | | | | gamma chains C-terminal |
| | | | | | | domain proteins. |
| 1185 | BL00514H | 14.95 | 7.545e−13 | 426-450 | BL00514 | Fibrinogen beta and |
| | | | | | | gamma chains C-terminal |
| | | | | | | domain proteins. |
| 1187 | BL50002A | 14.19 | 4.750e−12 | 339-357 | BL50002 | Src homology 3 (SH3) |
| | | | | | | domain proteins |
| | | | | | | profile. |
| 1187 | PR00452B | 11.65 | 5.500e−09 | 349-364 | PR00452 | SH3 DOMAIN SIGNATURE |
| 1188 | BL00663A | 11.51 | 8.364e−22 | 44-64 | BL00663 | Vinculin family talin- |
| | | | | | | binding region |
| | | | | | | proteins. |
| 1188 | BL00663B | 27.86 | 1000e−40 | 69-122 | BL00663 | Vinculin family talin- |
| | | | | | | binding region |
| | | | | | | proteins. |
| 1188 | BL00663C | 22.59 | 1.000e−40 | 131-185 | BL00663 | Vinculin family talin- |
| | | | | | | binding region |
| | | | | | | proteins. |
| 1188 | BL00663D | 24.77 | 1.000e−40 | 206-252 | BL00663 | Vinculin family talin- |
| | | | | | | binding region |
| | | | | | | proteins. |
| 1188 | BL00663E | 21.19 | 2.200e−33 | 254-291 | BL00663 | Vinculin family talin- |
| | | | | | | binding region |
| | | | | | | proteins. |
| 1188 | BL00663F | 20.78 | 7.000e−40 | 321-361 | BL00663 | Vinculin family talin- |
| | | | | | | binding region |
| | | | | | | proteins. |
| 1188 | BL00663G | 24.17 | 1.000e−40 | 393-442 | BL00663 | Vinculin family talin- |
| | | | | | | binding region |
| 1188 | BL00663H | 27.09 | 1.000e−40 | 465-517 | BL00663 | Vinculin family talin- |
| | | | | | | binding region |
| | | | | | | proteins. |
| 1188 | BL00663I | 27.27 | 1.000e−40 | 543-596 | BL00663 | Vinculin family talin- |
| | | | | | | binding region |
| | | | | | | proteins. |
| 1188 | BL00663J | 18.16 | 1.000e−40 | 725-761 | BL00663 | Vinculin family talin- |
| | | | | | | binding region |
| | | | | | | proteins. |
| 1188 | BL00663K | 21.52 | 1.000e−40 | 770-824 | BL00663 | Vinculin family talin- |
| | | | | | | binding region |
| | | | | | | proteins. |
| 1188 | BL00663L | 20.67 | 9.053e−38 | 837-872 | BL00663 | Vinculin family talin- |
| | | | | | | binding region |
| | | | | | | proteins. |
| 1188 | PR00805A | 11.15 | 1.692e−31 | 36-60 | PR00805 | ALPHA-CATENIN SIGNATURE |
| 1188 | PR00805B | 14.82 | 1.409e−25 | 134-152 | PR00805 | ALPHA-CATENIN SIGNATURE |
| 1188 | PR00805C | 8.43 | 1.300e−23 | 153-171 | PR00805 | ALPHA-CATENIN SIGNATURE |
| 1188 | PR00805D | 7.82 | 1.257e−31 | 329-353 | PR00805 | ALPHA-CATENIN SIGNATURE |
| 1188 | PR00805E | 10.38 | 1.375e−27 | 397-417 | PR00805 | ALPHA-CATENIN SIGNATURE |
| 1188 | PR00805F | 11.72 | 1.375e−31 | 518-542 | PR00805 | ALPHA-CATENIN SIGNATURE |
| 1188 | PR00805G | 11.78 | 1.000e−31 | 633-657 | PR00805 | ALPHA-CATENIN SIGNATURE |
| 1188 | PR00805H | 14.97 | 1.161e−31 | 735-757 | PR00805 | ALPHA-CATENIN SIGNATURE |
| 1188 | PR00805I | 12.05 | 1.188e−32 | 799-823 | PR00805 | ALPHA-CATENIN SIGNATURE |
| 1188 | PR00805J | 7.44 | 2.800e−25 | 879-897 | PR00805 | ALPHA-CATENIN SIGNATURE |
| 1188 | PR00806C | 11.07 | 2.258e−09 | 734-751 | PR00806 | VINCULIN SIGNATURE |
| 1188 | PR00929C | 5.26 | 9.069e−09 | 71-81 | PR00929 | AT-HOOK-LIKE DOMAIN |
| | | | | | | SIGNATURE |
| 1189 | BL00223A | 15.59 | 1.911e−11 | 78-111 | BL00223 | Annexins repeat |
| | | | | | | proteins domain |
| | | | | | | proteins. |
| 1189 | BL00223C | 24.79 | 5.725e−40 | 65-119 | BL00223 | Annexins repeat |
| | | | | | | proteins domain |
| | | | | | | proteins. |
| 1189 | PR00196C | 10.36 | 6.311e−13 | 82-103 | PR00196 | ANNEXIN FAMILY |
| | | | | | | SIGNATURE |
| 1189 | PR00196D | 21.86 | 6.400e−09 | 6-32 | PR00196 | ANNEXIN FAMILY |
| | | | | | | SIGNATURE |
| 1189 | PR00196E | 9.19 | 7.128e−18 | 86-106 | PR00196 | ANNEXIN FAMILY |
| | | | | | | SIGNATURE |
| 1189 | PR00196F | 13.89 | 1.000e−14 | 114-129 | PR00196 | ANNEXIN FAMILY |
| | | | | | | SIGNATURE |
| 1189 | PR00196G | 11.72 | 1.439e−11 | 130-143 | PR00196 | ANNEXIN FAMILY |
| | | | | | | SIGNATURE |
| 1189 | PR00197D | 7.50 | 1.297e−10 | 82-103 | PR00197 | ANNEXIN TYPE I |
| | | | | | | SIGNATURE |
| 1189 | PR00197F | 9.03 | 3.314e−17 | 86-106 | PR00197 | ANNEXIN TYPE I |
| | | | | | | SIGNATURE |
| 1189 | PR00198D | 7.65 | 1.175e−09 | 82-103 | PR00198 | ANNEXIN TYPE II |
| | | | | | | SIGNATURE |
| 1189 | PR00198G | 8.09 | 3.132e−14 | 86-106 | PR00198 | ANNEXIN TYPE II |
| | | | | | | SIGNATURE |
| 1189 | PR00198H | 12.05 | 9.663e−09 | 130-143 | PR00198 | ANNEXIN TYPE II |
| | | | | | | SIGNATURE |
| 1189 | PR00199D | 5.65 | 8.699e−11 | 82-103 | PR00199 | ANNEXIN TYPE III |
| | | | | | | SIGNATURE |
| 1189 | PR00199G | 9.09 | 2.588e−20 | 87-112 | PR00199 | ANNEXIN TYPE III |
| | | | | | | SIGNATURE |
| 1189 | PR00199H | 12.62 | 3.000e−15 | 130-143 | PR00199 | ANNEXIN TYPE III |
| | | | | | | SIGNATURE |
| 1189 | PR00200E | 10.00 | 1.489e−11 | 82-103 | PR00200 | ANNEXIN TYPE IV |
| | | | | | | SIGNATURE |
| 1189 | PR00200G | 9.43 | 1.000e−18 | 86-112 | PR00200 | ANNEXIN TYPE IV |
| | | | | | | SIGNATURE |
| 1189 | PR00200H | 13.68 | 2.987e−11 | 130-143 | PR00200 | ANNEXIN TYPE IV |
| | | | | | | SIGNATURE |
| 1189 | PR00201D | 10.49 | 8.981e−09 | 82-103 | PR00201 | ANNEXIN TYPE V |
| | | | | | | SIGNATURE |
| 1189 | PR00201G | 11.02 | 1.625e−21 | 86-112 | PR00201 | ANNEXIN TYPE V |
| | | | | | | SIGNATURE |
| 1189 | PR00201H | 12.04 | 8.024e−11 | 130-143 | PR00201 | ANNEXIN TYPE V |
| | | | | | | SIGNATURE |
| 1189 | PR00202G | 8.01 | 9.538e−21 | 86-112 | PR00202 | ANNEXIN TYPE VI |
| | | | | | | SIGNATURE |
| 1189 | PR00202H | 9.20 | 2.800e−10 | 130-143 | PR00202 | ANNEXIN TYPE VI |
| | | | | | | SIGNATURE |
| 1190 | BL00593A | 13.25 | 6.700e−30 | 38-63 | BL00593 | Heme oxygenase |
| | | | | | | proteins. |
| 1190 | BL00593B | 13.38 | 1.000e−40 | 72-115 | BL00593 | Heme oxygenase |
| | | | | | | proteins. |
| 1190 | BL00593C | 10.02 | 1.000e−40 | 144-188 | BL00593 | Heme oxygenase |
| | | | | | | proteins. |
| 1190 | BL00593D | 19.91 | 1.000e−40 | 192-244 | BL00593 | Heme oxygenase |
| | | | | | | proteins. |
| 1190 | BL00593E | 13.32 | 4.000e−24 | 292-314 | BL00593 | Heme oxygenase |
| | | | | | | proteins. |
| 1190 | PR00088A | 13.37 | 1.237e−29 | 38-63 | PR00088 | HAEM OXYGENASE |
| | | | | | | SIGNATURE |
| 1190 | PR00088B | 14.55 | 5.615e−29 | 73-98 | PR00088 | HAEM OXYGENASE |
| | | | | | | SIGNATURE |
| 1190 | PR00088C | 13.00 | 7.395e−30 | 99-124 | PR00088 | HAEM OXYGENASE |
| | | | | | | SIGNATURE |
| 1190 | PR00088D | 16.75 | 1.000e−27 | 152-173 | PR00088 | HAEM OXYGENASE |
| | | | | | | SIGNATURE |
| 1190 | PR00088E | 11.90 | 4.900e−25 | 174-195 | PR00088 | HAEM OXYGENASE |
| | | | | | | SIGNATURE |
| 1190 | PR00088F | 12.62 | 3.025e−24 | 196-217 | PR00088 | HAEM OXYGENASE |
| | | | | | | SIGNATURE |
| 1190 | PR00088G | 10.67 | 7.429e−21 | 228-246 | PR00088 | HAEM OXYGENASE |
| | | | | | | SIGNATURE |
| 1193 | BL00388A | 23.14 | 1.257e−33 | 15-60 | BL00388 | Proteasome A-type |
| | | | | | | subunits proteins. |
| 1193 | BL00388B | 31.38 | 7.429e−28 | 74-115 | BL00388 | Proteasome A-type |
| | | | | | | subunits proteins. |
| 1193 | BL00388C | 18.79 | 9.710e−17 | 129-150 | BL00388 | Proteasome A-type |
| | | | | | | subunits proteins. |
| 1193 | BL00388D | 20.71 | 3.382e−20 | 157-187 | BL00388 | Proteasome A-type |
| | | | | | | subunits proteins. |
| 1193 | PF00227 | 14.68 | 5.950e−09 | 22-33 | PF00227 | Proteasome A-type and |
| | | | | | | B-type. |
| 1194 | BL00061B | 25.79 | 2.636e−17 | 219-256 | BL00061 | Short-chain |
| | | | | | | dehydrogenases/reductases |
| | | | | | | family proteins. |
| 1194 | PR00080C | 17.16 | 8.714e−13 | 239-258 | PR00080 | ALCOHOL DEHYDROGENASE |
| | | | | | | SUPERFAMILY SIGNATURE |
| 1194 | PR00081A | 10.53 | 5.979e−10 | 91-108 | PR00081 | GLUCOSE/RIBITOL |
| | | | | | | DEHYDROGENASE FAMILY |
| | | | | | | SIGNATURE |
| 1194 | PR00081C | 15.13 | 9.654e−11 | 213-229 | PR00081 | GLUCOSE/RIBITOL |
| | | | | | | DEHYDROGENASE FAMILY |
| | | | | | | SIGNATURE |
| 1194 | PR00081D | 15.80 | 1.231e−10 | 239-258 | PR00081 | GLUCOSE/RIBITOL |
| | | | | | | DEHYDROGENASE FAMILY |
| | | | | | | SIGNATURE |
| 1195 | BL00326A | 14.01 | 9.686e−09 | 34-67 | BL00326 | Tropomyosins proteins. |
| 1197 | PF00651 | 15.00 | 7.857e−10 | 345-357 | PF00651 | BTB (also known as BR- |
| | | | | | | C/Ttk) domain proteins. |
| 1199 | PR00001A | 12.78 | 9.379e−15 | 38-51 | PR00001 | COAGULATION FACTOR GLA |
| | | | | | | DOMAIN SIGNATURE |
| 1199 | PR00001B | 10.75 | 6.932e−13 | 52-65 | PR00001 | COAGULATION FACTOR GLA |
| | | | | | | DOMAIN SIGNATURE |
| 1199 | PR00001C | 16.60 | 2.459e−11 | 66-80 | PR00001 | COAGULATION FACTOR GLA |
| | | | | | | DOMAIN SIGNATURE |
| 1200 | PD00289 | 9.97 | 4.103e−09 | 160-173 | PD00289 | PROTEIN SH3 DOMAIN |
| | | | | | | REPEAT PRESYNA. |
| 1201 | BL00107A | 18.39 | 3.118e−20 | 733-763 | BL00107 | Protein kinases ATP- |
| | | | | | | binding region |
| | | | | | | proteins. |
| 1201 | BL00107B | 13.31 | 8.615e−12 | 803-818 | BL00107 | Protein kinases ATP- |
| | | | | | | binding region |
| | | | | | | proteins. |
| 1201 | BL00239B | 25.15 | 9.224e−21 | 667-714 | BL00239 | Receptor tyrosine |
| | | | | | | kinase class II |
| | | | | | | proteins. |
| 1201 | BL00239C | 18.75 | 7.474e−13 | 720-742 | BL00239 | Receptor tyrosine |
| | | | | | | kinase class II |
| | | | | | | proteins. |
| 1201 | BL00239D | 16.81 | 2.373e−11 | 745-770 | BL00239 | Receptor tyrosine |
| | | | | | | kinase class II |
| | | | | | | proteins. |
| 1201 | BL00239E | 17.14 | 4.682e−24 | 775-824 | BL00239 | Receptor tyrosine |
| | | | | | | kinase class II |
| | | | | | | proteins. |
| 1201 | BL00239F | 28.15 | 2.929e−20 | 829-873 | BL00239 | Receptor tyrosine |
| | | | | | | kinase class II |
| | | | | | | proteins. |
| 1201 | BL00240E | 11.56 | 7.808e−17 | 719-756 | BL00240 | Receptor tyrosine |
| | | | | | | kinase class III |
| | | | | | | proteins. |
| 1201 | BL00240F | 17.74 | 9.241e−19 | 774-821 | BL00240 | Receptor tyrosine |
| | | | | | | kinase class III |
| | | | | | | proteins. |
| 1201 | BL00240G | 28.45 | 2.611e−11 | 821-873 | BL00240 | Receptor tyrosine |
| | | | | | | kinase class III |
| | | | | | | proteins. |
| 1201 | BL00790A | 19.74 | 5.371e−17 | 20-41 | BL00790 | Receptor tyrosine |
| | | | | | | kinase class V |
| | | | | | | proteins. |
| 1201 | BL00790B | 21.59 | 1.000e−40 | 53-104 | BL00790 | Receptor tyrosine |
| | | | | | | kinase class V |
| | | | | | | proteins. |
| 1201 | BL00790C | 16.65 | 1.000e−40 | 161-214 | BL00790 | Receptor tyrosine |
| | | | | | | kinase class V |
| | | | | | | proteins. |
| 1201 | BL00790D | 12.41 | 2.703e−22 | 238-262 | BL00790 | Receptor tyrosine |
| | | | | | | kinase class V |
| | | | | | | proteins. |
| 1201 | BL00790E | 29.58 | 6.400e−39 | 271-318 | BL00790 | Receptor tyrosine |
| | | | | | | kinase class V |
| | | | | | | proteins. |
| 1201 | BL00790F | 15.90 | 7.250e−22 | 337-363 | BL00790 | Receptor tyrosine |
| | | | | | | kinase class V |
| | | | | | | proteins. |
| 1201 | BL00790G | 22.06 | 2.846e−34 | 373-416 | BL00790 | Receptor tyrosine |
| | | | | | | kinase class V |
| | | | | | | proteins. |
| 1201 | BL00790H | 13.42 | 1.771e−23 | 452-477 | BL00790 | Receptor tyrosine |
| | | | | | | kinase class V |
| | | | | | | proteins. |
| 1201 | BL00790I | 20.01 | 8.941e−27 | 499-529 | BL00790 | Receptor tyrosine |
| | | | | | | kinase class V |
| | | | | | | proteins. |
| 1201 | BL00790J | 14.21 | 1.000e−40 | 588-627 | BL00790 | Receptor tyrosine |
| | | | | | | kinase class V |
| | | | | | | proteins. |
| 1201 | BL00790K | 9.30 | 1.000e−40 | 640-693 | BL00790 | Receptor tyrosine |
| | | | | | | kinase class V |
| | | | | | | proteins. |
| 1201 | BL00790L | 11.16 | 3.423e−24 | 704-723 | BL00790 | Receptor tyrosine |
| | | | | | | kinase class V |
| | | | | | | proteins. |
| 1201 | BL00790M | 8.74 | 4.977e−26 | 724-745 | BL00790 | Receptor tyrosine |
| | | | | | | kinase class V |
| | | | | | | proteins. |
| 1201 | BL00790N | 13.25 | 8.071e−31 | 746-772 | BL00790 | Receptor tyrosine |
| | | | | | | kinase class V |
| | | | | | | proteins. |
| 1201 | BL00790O | 7.68 | 1.000e−40 | 782-814 | BL00790 | Receptor tyrosine |
| | | | | | | kinase class V |
| | | | | | | proteins. |
| 1201 | BL00790P | 12.33 | 1.281e−30 | 815-839 | BL00790 | Receptor tyrosine |
| | | | | | | kinase class V |
| | | | | | | proteins. |
| 1201 | BL00790Q | 15.61 | 1.000e−40 | 840-888 | BL00790 | Receptor tyrosine |
| | | | | | | kinase class V |
| | | | | | | proteins. |
| 1201 | BL00790R | 16.20 | 4.000e−39 | 931-974 | BL00790 | Receptor tyrosine |
| | | | | | | kinase class V |
| | | | | | | proteins. |
| 1201 | BL50001B | 17.40 | 3.647e−13 | 730-750 | BL50001 | Src homology 2 (SH2) |
| | | | | | | domain proteins |
| | | | | | | profile. |
| 1201 | BL50001C | 10.17 | 1.000e−09 | 784-794 | BL50001 | Src homology 2 (SH2) |
| | | | | | | domain proteins |
| | | | | | | profile. |
| 1201 | PR00014D | 12.04 | 3.077e−12 | 506-520 | PR00014 | FIBRONECTIN TYPE III |
| | | | | | | REPEAT SIGNATURE |
| 1201 | PR00109A | 15.00 | 1.409e−12 | 696-709 | PR00109 | TYROSINE KINASE |
| | | | | | | CATALYTIC DOMAIN |
| | | | | | | SIGNATURE |
| 1201 | PR00109B | 12.27 | 1.000e−19 | 733-751 | PR00109 | TYROSINE KINASE |
| | | | | | | CATALYTIC DOMAIN |
| | | | | | | SIGNATURE |
| 1201 | PR00109C | 12.85 | 5.235e−12 | 785-795 | PR00109 | TYROSINE KINASE |
| | | | | | | CATALYTIC DOMAIN |
| | | | | | | SIGNATURE |
| 1201 | PR00109D | 17.04 | 2.038e−20 | 804-826 | PR00109 | TYROSINE KINASE |
| | | | | | | CATALYTIC DOMAIN |
| | | | | | | SIGNATURE |
| 1201 | PR00109E | 14.41 | 8.500e−18 | 848-870 | PR00109 | TYROSINE KINASE |
| | | | | | | CATALYTIC DOMAIN |
| | | | | | | SIGNATURE |
| 1203 | DM00215 | 19.43 | 9.390e−09 | 157-189 | DM00215 | PROLINE-RICH PROTEIN 3. |
| 1203 | PR00049D | 0.00 | 7.254e−09 | 175-189 | PR00049 | WILM'S TUMOUR PROTEIN |
| | | | | | | SIGNATURE |
| 1204 | BL00225A | 13.82 | 3.127e−09 | 1133-1153 | BL00225 | Crystallins beta and |
| | | | | | | gamma ‘Greek key’ motif |
| | | | | | | proteins. |
| 1204 | BL00225A | 13.82 | 5.829e−12 | 1417-1437 | BL00225 | Crystallins beta and |
| | | | | | | gamma ‘Greek key’ motif |
| | | | | | | proteins. |
| 1204 | BL00225B | 18.06 | 2.575e−19 | 1270-1304 | BL00225 | Crystallins beta and |
| | | | | | | gamma ‘Greek key’ motif |
| | | | | | | proteins. |
| 1204 | BL00225B | 18.06 | 4.808e−14 | 970-1004 | BL00225 | Crystallins beta and |
| | | | | | | gamma ‘Greek key’ motif |
| | | | | | | proteins. |
| 1204 | BL00225B | 18.06 | 5.500e−14 | 1451-1485 | BL00225 | Crystallins beta and |
| | | | | | | gamma ‘Greek key’ motif |
| | | | | | | proteins. |
| 1204 | BL00225B | 18.06 | 7.517e−24 | 1179-1213 | BL00225 | Crystallins beta and |
| | | | | | | gamma ‘Greek key’ motif |
| | | | | | | proteins. |
| 1204 | BL00225B | 18.06 | 8.200e−19 | 1072-1106 | BL00225 | Crystallins beta and |
| | | | | | | gamma ‘Greek key’ motif |
| | | | | | | proteins. |
| 1204 | BL00225B | 18.06 | 8.297e−20 | 1361-1395 | BL00225 | Crystallins beta and |
| | | | | | | gamma ‘Greek key’ motif |
| | | | | | | proteins. |
| 1205 | BL00266B | 24.48 | 8.457e−21 | 19-56 | BL00266 | Somatotropin, prolactin |
| | | | | | | and related hormones |
| | | | | | | proteins. |
| 1205 | PR00836A | 14.40 | 2.800e−13 | 19-32 | PR00836 | SOMATOTROPIN HORMONE |
| | | | | | | FAMILY SIGNATURE |
| 1205 | PR00836B | 16.59 | 5.814e−10 | 41-59 | PR00836 | SOMATOTROPIN HORMONE |
| | | | | | | FAMILY SIGNATURE |
| 1205 | PR00836C | 11.95 | 1.000e−12 | 119-135 | PR00836 | SOMATOTROPIN HORMONE |
| | | | | | | FAMILY SIGNATURE |
| 1206 | BL00046 | 12.95 | 1.000e−40 | 59-113 | BL00046 | Histone H2A proteins. |
| 1206 | PR00620A | 11.03 | 2.636e−27 | 40-62 | PR00620 | HISTONE H2A SIGNATURE |
| 1206 | PR00620B | 8.47 | 1.750e−20 | 69-84 | PR00620 | HISTONE H2A SIGNATURE |
| 1206 | PR00620C | 10.19 | 2.125e−17 | 84-97 | PR00620 | HISTONE H2A SIGNATURE |
| 1206 | PR00620D | 11.39 | 1.692e−19 | 98-112 | PR00620 | HISTONE H2A SIGNATURE |
| 1206 | PR00620E | 8.91 | 2.500e−16 | 112-124 | PR00620 | HISTONE H2A SIGNATURE |
| 1206 | PR00620F | 7.29 | 7.750e−22 | 126-144 | PR00620 | HISTONE H2A SIGNATURE |
| 1208 | BL00309A | 19.16 | 7.545e−20 | 75-99 | BL00309 | Vertebrate galactoside- |
| | | | | | | binding lectin |
| | | | | | | proteins. |
| 1208 | BL00309B | 12.11 | 6.000e−16 | 101-115 | BL00309 | Vertebrate galactoside- |
| | | | | | | binding lectin |
| | | | | | | proteins. |
| 1208 | BL00309C | 18.65 | 5.000e−24 | 123-147 | BL00309 | Vertebrate galactoside- |
| | | | | | | binding lectin |
| | | | | | | proteins. |
| 1209 | PF00075B | 12.56 | 4.073e−10 | 260-270 | PF00075 | RNase H. |
| 1209 | PF00075C | 11.58 | 9.786e−11 | 295-306 | PF00075 | RNase H. |
| 1209 | PF00075D | 10.71 | 7.000e−11 | 327-337 | PF00075 | RNase H. |
| 1209 | PF00075F | 12.87 | 4.750e−11 | 685-695 | PF00075 | RNase H. |
| 1209 | PF00075J | 15.78 | 8.350e−11 | 980-997 | PF00075 | RNase H. |
| 1209 | PF00429 | 31.08 | 3.492e−17 | 1105-1154 | PF00429 | ENV polyprotein (coat |
| | | | | | | polyprotein). |
| 1210 | BL00272C | 8.27 | 8.326e−09 | 95-106 | BL00272 | Snake toxins proteins. |
| 1210 | BL00983C | 12.69 | 4.981e−09 | 91-106 | BL00983 | Ly-6/u-PAR domain |
| | | | | | | proteins. |
| 1211 | BL00139A | 10.29 | 7.261e−14 | 320-329 | BL00139 | Eukaryotic thiol |
| | | | | | | (cysteine) proteases |
| | | | | | | cysteine proteins. |
| 1211 | BL00139C | 9.23 | 1.000e−11 | 461-470 | BL00139 | Eukaryotic thiol |
| | | | | | | (cysteine) proteases |
| | | | | | | cysteine proteins. |
| 1211 | BL00139D | 9.24 | 5.125e−17 | 477-493 | BL00139 | Eukaryotic thiol |
| | | | | | | (cysteine) proteases |
| | | | | | | cysteine proteins. |
| 1211 | PR00705A | 10.55 | 6.294e−16 | 320-335 | PR00705 | PAPAIN CYSTEINE |
| | | | | | | PROTEASE (C1) FAMILY |
| | | | | | | SIGNATURE |
| 1211 | PR00705B | 10.22 | 1.844e−09 | 462-472 | PR00705 | PAPAIN CYSTEINE |
| | | | | | | PROTEASE (C1) FAMILY |
| | | | | | | SIGNATURE |
| 1214 | BL00282 | 16.88 | 6.294e−11 | 12-34 | BL00282 | Kazal serine protease |
| | | | | | | inhibitors family |
| | | | | | | proteins. |
| 1216 | BL01177C | 17.39 | 2.875e−20 | 109-127 | BL01177 | Anaphylatoxin domain |
| | | | | | | proteins. |
| 1216 | BL01187A | 9.98 | 1.000e−12 | 100-111 | BL01187 | Calcium-binding EGF- |
| | | | | | | like domain proteins |
| | | | | | | pattern proteins. |
| 1216 | BL01187A | 9.98 | 6.625e−09 | 58-69 | BL01187 | Calcium-binding EGF- |
| | | | | | | like domain proteins |
| | | | | | | pattern proteins. |
| 1216 | BL01187B | 12.04 | 3.250e−14 | 76-91 | BL01187 | Calcium-binding EGF- |
| | | | | | | like domain proteins |
| | | | | | | pattern proteins. |
| 1216 | BL01187B | 12.04 | 6.478e−13 | 115-130 | BL01187 | Calcium-binding EGF- |
| | | | | | | like domain proteins |
| | | | | | | pattern proteins. |
| 1216 | PR00010A | 11.79 | 9.308e−09 | 102-113 | PR00010 | TYPE II EGF-LIKE |
| | | | | | | SIGNATURE |
| 1216 | PR00010C | 11.16 | 5.714e−09 | 81-91 | PR00010 | TYPE II EGF-LIKE |
| | | | | | | SIGNATURE |
| 1218 | BL00226D | 19.10 | 1.000e−40 | 31-77 | BL00226 | Intermediate filaments |
| | | | | | | proteins. |
| 1219 | BL00107A | 18.39 | 6.192e−12 | 211-241 | BL00107 | Protein kinases ATP- |
| | | | | | | binding region |
| | | | | | | proteins. |
| 1219 | BL00239B | 25.15 | 8.909e−17 | 146-193 | BL00239 | Receptor tyrosine |
| | | | | | | kinase class II |
| | | | | | | proteins. |
| 1219 | BL00240D | 23.07 | 3.483e−09 | 135-189 | BL00240 | Receptor tyrosine |
| | | | | | | kinase class III |
| | | | | | | proteins. |
| 1219 | BL00240E | 11.56 | 6.185e−12 | 197-234 | BL00240 | Receptor tyrosine |
| | | | | | | kinase class III |
| | | | | | | proteins. |
| 1219 | BL00479C | 12.01 | 2.000e−09 | 263-275 | BL00479 | Phorbol esters/ |
| | | | | | | diacylglycerol binding |
| | | | | | | domain proteins. |
| 1219 | BL00790M | 8.74 | 3.571e−09 | 202-223 | BL00790 | Receptor tyrosine |
| | | | | | | kinase class V |
| | | | | | | proteins. |
| 1219 | PR00109A | 15.00 | 1.486e−09 | 175-188 | PR00109 | TYROSINE KINASE |
| | | | | | | CATALYTIC DOMAIN |
| | | | | | | SIGNATURE |
| 1219 | PR00109B | 12.27 | 5.596e−13 | 211-229 | PR00109 | TYROSINE KINASE |
| | | | | | | CATALYTIC DOMAIN |
| | | | | | | SIGNATURE |
| 1221 | BL00421E | 20.97 | 5.125e−16 | 41-70 | BL00421 | Transmembrane 4 family |
| | | | | | | proteins. |
| 1221 | PR00259D | 13.50 | 2.241e−21 | 44-70 | PR00259 | TRANSMEMBRANE FOUR |
| | | | | | | FAMILY SIGNATURE |
| 1222 | BL00540B | 18.82 | 1.000e−40 | 50-104 | BL00540 | Ferritin iron-binding |
| | | | | | | regions proteins. |
| 1222 | BL00540C | 13.00 | 7.750e−14 | 115-126 | BL00540 | Ferritin iron-binding |
| | | | | | | regions proteins. |
| 1223 | BL00824B | 9.21 | 7.750e−22 | 89-108 | BL00824 | Elongation factor 1 |
| | | | | | | beta/beta'/delta chain |
| | | | | | | proteins. |
| 1224 | BL00266A | 15.69 | 9.000e−19 | 30-56 | BL00266 | Somatotropin, prolactin |
| | | | | | | and related hormones |
| | | | | | | proteins. |
| 1224 | BL00266B | 24.48 | 7.261e−16 | 74-111 | BL00266 | Somatotropin, prolactin |
| | | | | | | and related hormones |
| | | | | | | proteins. |
| 1224 | PR00836A | 14.40 | 9.379e−11 | 74-87 | PR00836 | SOMATOTROPIN HORMONE |
| | | | | | | FAMILY SIGNATURE |
| 1225 | PF00084C | 11.25 | 3.571e−10 | 34-43 | PF00084 | Sushi domain proteins |
| | | | | | | (SCR repeat proteins. |
| 1227 | BL00475C | 13.06 | 3.700e−17 | 57-73 | BL00475 | Ribosomal protein L15 |
| | | | | | | proteins. |
| 1228 | PR00702G | 9.63 | 2.625e−09 | 22-45 | PR00702 | ACRIFLAVIN RESISTANCE |
| | | | | | | PROTEIN FAMILY |
| | | | | | | SIGNATURE |
| 1231 | PD02269B | 18.48 | 2.667e−27 | 74-100 | PD02269 | CYTIDINE DEAMINASE |
| | | | | | | HYDROLASE ZINC AMINOHY. |
| 1231 | PD02269C | 16.36 | 7.882e−17 | 119-131 | PD02269 | CYTIDINE DEAMINASE |
| | | | | | | HYDROLASE ZINC AMINOHY. |
| 1232 | BL00061A | 9.41 | 4.600e−13 | 105-115 | BL00061 | Short-chain |
| | | | | | | dehydrogenases/reductases |
| | | | | | | family proteins. |
| 1232 | PR00080A | 9.32 | 3.143e−12 | 105-116 | PR00080 | ALCOHOL DEHYDROGENASE |
| | | | | | | SUPERFAMILY SIGNATURE |
| 1232 | PR00081B | 10.38 | 8.800e−14 | 105-116 | PR00081 | GLUCOSE/RIBITOL |
| | | | | | | DEHYDROGENASE FAMILY |
| | | | | | | SIGNATURE |
| 1233 | BL00064A | 21.16 | 1.000e−33 | 48-85 | BL00064 | L-lactate dehydrogenase |
| | | | | | | proteins. |
| 1233 | BL00957A | 8.31 | 9.609e−09 | 48-63 | BL00957 | NAD-dependent glycerol- |
| | | | | | | 3-phosphate |
| | | | | | | dehydrogenase proteins. |
| 1233 | PR00086A | 16.12 | 4.000e−26 | 48-72 | PR00086 | L-LACTATE DEHYDROGENASE |
| | | | | | | SIGNATURE |
| 1233 | PR00086B | 13.67 | 4.462e−24 | 73-97 | PR00086 | L-LACTATE DEHYDROGENASE |
| | | | | | | SIGNATURE |
| 1234 | BL00353B | 11.47 | 1.000e−40 | 65-114 | BL00353 | HMG1/2 proteins. |
| 1234 | BL00353C | 14.83 | 5.680e−24 | 115-161 | BL00353 | HMG1/2 proteins. |
| 1234 | BL00353C | 14.83 | 7.254e−09 | 31-77 | BL00353 | HMG1/2 proteins. |
| 1234 | PR00886A | 10.08 | 2.929e−30 | 27-49 | PR00886 | HIGH MOBILITY GROUP |
| | | | | | | (HMG1/HMG2) PROTEIN |
| | | | | | | SIGNATURE |
| 1234 | PR00886B | 9.88 | 6.625e−27 | 50-70 | PR00886 | HIGH MOBILITY GROUP |
| | | | | | | (HMG1/HMG2) PROTEIN |
| | | | | | | SIGNATURE |
| 1234 | PR00886C | 11.84 | 1.500e−22 | 97-115 | PR00886 | HIGH MOBILITY GROUP |
| | | | | | | (HMG1/HMG2) PROTEIN |
| | | | | | | SIGNATURE |
| 1234 | PR00886D | 15.72 | 4.830e−14 | 115-134 | PR00886 | HIGH MOBILITY GROUP |
| | | | | | | (HMG1/HMG2) PROTEIN |
| | | | | | | SIGNATURE |
| 1234 | PR00886E | 8.85 | 9.316e−09 | 50-70 | PR00886 | HIGH MOBILITY GROUP |
| | | | | | | (HMG1/HMG2) PROTEIN |
| | | | | | | SIGNATURE |
| 1235 | BL00740A | 13.87 | 7.200e−10 | 64-76 | BL00740 | MAM domain proteins. |
| 1235 | PR00020A | 18.17 | 6.684e−10 | 62-80 | PR00020 | MAM DOMAIN SIGNATURE |
| 1236 | BL01187A | 9.98 | 3.250e−09 | 68-79 | BL01187 | Calcium-binding EGF- |
| | | | | | | like domain proteins |
| | | | | | | pattern proteins. |
| 1236 | BL01187A | 9.98 | 3.571e−10 | 27-38 | BL01187 | Calcium-binding EGF- |
| | | | | | | like domain proteins |
| | | | | | | pattern proteins. |
| 1236 | BL01187B | 12.04 | 1.391e−13 | 46-61 | BL01187 | Calcium-binding EGF- |
| | | | | | | like domain proteins |
| | | | | | | pattern proteins. |
| 1236 | BL01187B | 12.04 | 2.000e−12 | 5-20 | BL01187 | Calcium-binding EGF- |
| | | | | | | like domain proteins |
| | | | | | | pattern proteins. |
| 1236 | BL01187B | 12.04 | 4.375e−09 | 86-101 | BL01187 | Calcium-binding EGF- |
| | | | | | | like domain proteins |
| | | | | | | pattern proteins. |
| 1236 | PR00010C | 11.16 | 7.000e−09 | 51-61 | PR00010 | TYPE II EGF-LIKE |
| | | | | | | SIGNATURE |
| 1236 | PR00907G | 11.63 | 6.514e−09 | 46-72 | PR00907 | THROMBOMODULIN |
| | | | | | | SIGNATURE |
| 1236 | PR00907G | 11.63 | 8.622e−09 | 5-31 | PR00907 | THROMBOMODULIN |
| | | | | | | SIGNATURE |
| 1237 | BL00287 | 17.35 | 1.947e−20 | 25-48 | BL00287 | Cysteine proteases |
| | | | | | | inhibitors proteins. |
| 1245 | DM00303A | 13.20 | 3.382e−11 | 52-101 | DM00303 | 6 LEA 11-MER REPEAT |
| | | | | | | REPEAT. |
| 1246 | BL00064C | 17.28 | 1.000e−40 | 18-62 | BL00064 | L-lactate dehydrogenase |
| | | | | | | proteins. |
| 1246 | BL00064D | 14.19 | 8.875e−32 | 63-92 | BL00064 | L-lactate dehydrogenase |
| | | | | | | proteins. |
| 1246 | BL00068C | 29.08 | 3.089e−17 | 14-60 | BL00068 | Malate dehydrogenase |
| | | | | | | proteins. |
| 1246 | PR00086C | 16.22 | 6.400e−25 | 15-35 | PR00086 | L-LACTATE DEHYDROGENASE |
| | | | | | | SIGNATURE |
| 1246 | PR00086D | 15.53 | 4.600e−24 | 39-57 | PR00086 | L-LACTATE DEHYDROGENASE |
| | | | | | | SIGNATURE |
| 1246 | PR00086E | 11.21 | 8.364e−18 | 69-82 | PR00086 | L-LACTATE DEHYDROGENASE |
| | | | | | | SIGNATURE |
| 1251 | BL00142 | 8.38 | 4.429e−09 | 430-440 | BL00142 | Neutral zinc |
| | | | | | | metallopeptidases, |
| | | | | | | zinc-binding region |
| | | | | | | proteins. |
| 1251 | PD01719A | 12.89 | 3.429e−14 | 594-621 | PD01719 | PRECURSOR GLYCOPROTEIN |
| | | | | | | SIGNAL RE. |
| 1251 | PD01719A | 12.89 | 9.408e−10 | 943-970 | PD01719 | PRECURSOR GLYCOPROTEIN |
| | | | | | | SIGNAL RE. |
| 1254 | BL00319C | 17.12 | 1.592e−09 | 43-76 | BL00319 | Amyloidogenic |
| | | | | | | glycoprotein |
| | | | | | | extracellular domain |
| | | | | | | proteins. |
| 1254 | BL00319C | 17.12 | 2.625e−10 | 106-139 | BL00319 | Amyloidogenic |
| | | | | | | glycoprotein |
| | | | | | | extracellular domain |
| | | | | | | proteins. |
| 1254 | BL00319C | 17.12 | 3.013e−09 | 100-133 | BL00319 | Amyloidogenic |
| | | | | | | glycoprotein |
| | | | | | | extracellular domain |
| | | | | | | proteins. |
| 1254 | BL00319C | 17.12 | 3.857e−12 | 101-134 | BL00319 | Amyloidogenic |
| | | | | | | glycoprotein |
| | | | | | | extracellular domain |
| | | | | | | proteins. |
| 1254 | BL00319C | 17.12 | 4.671e−09 | 107-140 | BL00319 | Amyloidogenic |
| | | | | | | glycoprotein |
| | | | | | | extracellular domain |
| | | | | | | proteins. |
| 1254 | BL00319C | 17.12 | 4.875e−10 | 97-130 | BL00319 | Amyloidogenic |
| | | | | | | glycoprotein |
| | | | | | | extracellular domain |
| | | | | | | proteins. |
| 1254 | BL00319C | 17.12 | 5.836e−11 | 44-77 | BL00319 | Amyloidogenic |
| | | | | | | glycoprotein |
| | | | | | | extracellular domain |
| | | | | | | proteins. |
| 1254 | BL00319C | 17.12 | 7.625e−10 | 104-137 | BL00319 | Amyloidogenic |
| | | | | | | glycoprotein |
| | | | | | | extracellular domain |
| | | | | | | proteins. |
| 1254 | BL00319C | 17.12 | 8.105e−09 | 98-131 | BL00319 | Amyloidogenic |
| | | | | | | glycoprotein |
| | | | | | | extracellular domain |
| | | | | | | proteins. |
| 1254 | BL00319C | 17.12 | 8.800e−13 | 102-135 | BL00319 | Amyloidogenic |
| | | | | | | glycoprotein |
| | | | | | | extracellular domain |
| | | | | | | proteins. |
| 1254 | BL00319C | 17.12 | 8.857e−12 | 105-138 | BL00319 | Amyloidogenic |
| | | | | | | glycoprotein |
| | | | | | | extracellular domain |
| | | | | | | proteins. |
| 1254 | BL00319C | 17.12 | 9.289e−09 | 39-72 | BL00319 | Amyloidogenic |
| | | | | | | glycoprotein |
| | | | | | | extracellular domain |
| | | | | | | proteins. |
| 1254 | BL00319C | 17.12 | 9.289e−09 | 96-129 | BL00319 | Amyloidogenic |
| | | | | | | glycoprotein |
| | | | | | | extracellular domain |
| | | | | | | proteins. |
| 1254 | BL00412D | 16.54 | 3.935e−10 | 66-116 | BL00412 | Neuromodulin (GAP-43) |
| | | | | | | proteins. |
| 1254 | BL00412D | 16.54 | 6.283e−10 | 93-143 | BL00412 | Neuromodulin (GAP-43) |
| | | | | | | proteins. |
| 1254 | BL00412D | 16.54 | 6.420e−11 | 68-118 | BL00412 | Neuromodulin (GAP-43) |
| | | | | | | proteins. |
| 1254 | BL00824B | 9.21 | 2.216e−09 | 107-126 | BL00824 | Elongation factor 1 |
| | | | | | | beta/beta′/delta chain |
| | | | | | | proteins. |
| 1254 | BL00824B | 9.21 | 2.338e−09 | 106-125 | BL00824 | Elongation factor 1 |
| | | | | | | beta/beta′/delta chain |
| | | | | | | proteins. |
| 1254 | BL00824B | 9.21 | 4.770e−09 | 105-124 | BL00824 | Elongation factor 1 |
| | | | | | | beta/beta′/delta chain |
| | | | | | | proteins. |
| 1254 | BL00824B | 9.21 | 5.257e−09 | 45-64 | BL00824 | Elongation factor 1 |
| | | | | | | beta/beta′/delta chain |
| | | | | | | proteins. |
| 1254 | BL00824B | 9.21 | 8.435e−10 | 102-121 | BL00824 | Elongation factor 1 |
| | | | | | | beta/beta′/delta chain |
| | | | | | | proteins. |
| 1254 | BL00884B | 12.47 | 9.664e−09 | 76-119 | BL00884 | Osteopontin proteins. |
| 1254 | BL01115D | 14.49 | 6.924e−09 | 14-45 | BL01115 | GTP-binding nuclear |
| | | | | | | protein ran proteins. |
| 1254 | PD00301A | 10.24 | 1.900e−09 | 116-126 | PD00301 | PROTEIN REPEAT MUSCLE |
| | | | | | | CALCIUM-BI. |
| 1254 | PD00919F | 11.63 | 5.107e−09 | 33-61 | PD00919 | CALCIUM-BINDING |
| | | | | | | PRECURSOR SIGNAL R. |
| 1254 | PF00922A | 19.17 | 4.951e−10 | 92-125 | PF00922 | Vesiculovirus |
| | | | | | | phosphoprotein. |
| 1254 | PF00922A | 19.17 | 5.966e−09 | 94-127 | PF00922 | Vesiculovirus |
| | | | | | | phosphoprotein. |
| 1254 | PF00922A | 19.17 | 6.690e−09 | 91-124 | PF00922 | Vesiculovirus |
| | | | | | | phosphoprotein. |
| 1254 | PF00922A | 19.17 | 9.561e−10 | 93-126 | PF00922 | Vesiculovirus |
| | | | | | | phosphoprotein. |
| 1255 | PR00121B | 7.83 | 9.250e−09 | 87-107 | PR00121 | SODIUM/POTASSIUM- |
| | | | | | | TRANSPORTING ATPASE |
| | | | | | | SIGNATURE |
| 1256 | BL00406B | 5.47 | 1.000e−40 | 72-126 | BL00406 | Actins proteins. |
| 1256 | PR00190A | 7.24 | 1.375e−13 | 15-24 | PR00190 | ACTIN SIGNATURE |
| 1256 | PR00190B | 9.98 | 1.000e−15 | 38-49 | PR00190 | ACTIN SIGNATURE |
| 1256 | PR00190C | 11.49 | 1.474e−30 | 50-72 | PR00190 | ACTIN SIGNATURE |
| 1256 | PR00190D | 19.23 | 1.450e−26 | 73-91 | PR00190 | ACTIN SIGNATURE |
| 1256 | PR00190E | 7.16 | 1.450e−19 | 104-117 | PR00190 | ACTIN SIGNATURE |
| 1258 | BL00389A | 11.02 | 7.188e−10 | 37-49 | BL00389 | ATP synthase delta |
| | | | | | | (OSCP) subunit |
| | | | | | | proteins. |
| 1258 | BL00389B | 17.02 | 6.211e−13 | 107-128 | BL00389 | ATP synthase delta |
| | | | | | | (OSCP) subunit |
| | | | | | | proteins. |
| 1258 | BL00389C | 20.13 | 6.760e−25 | 168-205 | BL00389 | ATP synthase delta |
| | | | | | | (OSCP) subunit |
| | | | | | | proteins. |
| 1258 | PR00125A | 16.03 | 8.364e−15 | 37-56 | PR00125 | ATP SYNTHASE DELTA |
| | | | | | | SUBUNIT SIGNATURE |
| 1258 | PR00125B | 12.78 | 8.125e−10 | 106-117 | PR00125 | ATP SYNTHASE DELTA |
| | | | | | | SUBUNIT SIGNATURE |
| 1258 | PR00125D | 11.00 | 5.345e−11 | 169-184 | PR00125 | ATP SYNTHASE DELTA |
| | | | | | | SUBUNIT SIGNATURE |
| 1258 | PR00125E | 13.56 | 9.250e−16 | 184-202 | PR00125 | ATP SYNTHASE DELTA |
| | | | | | | SUBUNIT SIGNATURE |
| 1260 | BL00678 | 9.67 | 1.947e−09 | 194-204 | BL00678 | Trp-Asp (WD) repeat |
| | | | | | | proteins proteins. |
| 1260 | BL00678 | 9.67 | 9.053e−09 | 290-300 | BL00678 | Trp-Asp (WD) repeat |
| | | | | | | proteins proteins. |
| 1260 | PR00320A | 16.74 | 2.862e−11 | 288-302 | PR00320 | G-PROTEIN BETA WD-40 |
| | | | | | | REPEAT SIGNATURE |
| 1260 | PR00320A | 16.74 | 2.976e−09 | 332-346 | PR00320 | G-PROTEIN BETA WD-40 |
| | | | | | | REPEAT SIGNATURE |
| 1260 | PR00320A | 16.74 | 5.714e−13 | 192-206 | PR00320 | G-PROTEIN BETA WD-40 |
| | | | | | | REPEAT SIGNATURE |
| 1260 | PR00320B | 12.19 | 1.783e−13 | 288-302 | PR00320 | G-PROTEIN BETA WD-40 |
| | | | | | | REPEAT SIGNATURE |
| 1260 | PR00320B | 12.19 | 5.114e−10 | 192-206 | PR00320 | G-PROTEIN BETA WD-40 |
| | | | | | | REPEAT SIGNATURE |
| 1260 | PR00320C | 13.01 | 1.783e−11 | 332-346 | PR00320 | G-PROTEIN BETA WD-40 |
| | | | | | | REPEAT SIGNATURE |
| 1260 | PR00320C | 13.01 | 3.400e−09 | 192-206 | PR00320 | G-PROTEIN BETA WD-40 |
| | | | | | | REPEAT SIGNATURE |
| 1260 | PR00320C | 13.01 | 9.640e−10 | 288-302 | PR00320 | G-PROTEIN BETA WD-40 |
| | | | | | | REPEAT SIGNATURE |
| 1261 | BL00678 | 9.67 | 7.158e−09 | 69-79 | BL00678 | Trp-Asp (WD) repeat |
| | | | | | | proteins proteins. |
| 1261 | PR00320A | 16.74 | 6.707e−09 | 203-217 | PR00320 | G-PROTEIN BETA WD-40 |
| | | | | | | REPEAT SIGNATURE |
| 1261 | PR00320A | 16.74 | 9.122e−09 | 67-81 | PR00320 | G-PROTEIN BETA WD-40 |
| | | | | | | REPEAT SIGNATURE |
| 1261 | PR00320B | 12.19 | 1.514e−10 | 67-81 | PR00320 | G-PROTEIN BETA WD-40 |
| | | | | | | REPEAT SIGNATURE |
| 1261 | PR00320C | 13.01 | 2.500e−09 | 67-81 | PR00320 | G-PROTEIN BETA WD-40 |
| | | | | | | REPEAT SIGNATURE |
| 1261 | PR00320C | 13.01 | 4.960e−10 | 203-217 | PR00320 | G-PROTEIN BETA WD-40 |
| | | | | | | REPEAT SIGNATURE |
| 1262 | BL00019A | 12.56 | 3.475e−13 | 73-83 | BL00019 | Actinin-type actin- |
| | | | | | | binding domain |
| | | | | | | proteins. |
| 1262 | BL00019B | 13.34 | 4.288e−10 | 2002-2024 | BL00019 | Actinin-type actin- |
| | | | | | | binding domain |
| | | | | | | proteins. |
| 1262 | BL00019B | 13.34 | 9.135e−10 | 98-120 | BL00019 | Actinin-type actin- |
| | | | | | | binding domain |
| | | | | | | proteins. |
| 1262 | BL00019C | 14.66 | 4.800e−30 | 146-181 | BL00019 | Actinin-type actin- |
| | | | | | | binding domain |
| | | | | | | proteins. |
| 1262 | BL00019D | 15.33 | 2.623e−13 | 2465-2494 | BL00019 | Actinin-type actin- |
| | | | | | | binding domain |
| | | | | | | proteins. |
| 1262 | BL00019D | 15.33 | 8.105e−09 | 210-239 | BL00019 | Actinin-type actin- |
| | | | | | | binding domain |
| | | | | | | proteins. |
| 1262 | BL00019F | 19.68 | 6.609e−09 | 182-203 | BL00019 | Actinin-type actin- |
| | | | | | | binding domain |
| | | | | | | proteins. |
| 1264 | BL00514C | 17.41 | 4.000e−29 | 59-95 | BL00514 | Fibrinogen beta and |
| | | | | | | gamma chains C-terminal |
| | | | | | | domain proteins. |
| 1264 | BL00514D | 15.35 | 7.511e−12 | 100-112 | BL00514 | Fibrinogen beta and |
| | | | | | | gamma chains C-terminal |
| | | | | | | domain proteins. |
| 1264 | BL00514E | 14.28 | 3.951e−10 | 119-135 | BL00514 | Fibrinogen beta and |
| | | | | | | gamma chains C-terminal |
| | | | | | | domain proteins. |
| 1264 | BL00514G | 15.98 | 2.268e−16 | 184-213 | BL00514 | Fibrinogen beta and |
| | | | | | | gamma chains C-terminal |
| | | | | | | domain proteins. |
| 1264 | BL00514H | 14.95 | 7.245e−15 | 217-241 | BL00514 | Fibrinogen beta and |
| | | | | | | gamma chains C-terminal |
| | | | | | | domain proteins. |
| 1265 | BL00038B | 16.97 | 4.273e−11 | 112-132 | BL00038 | Myc-type, ‘helix-loop- |
| | | | | | | helix’ dimerization |
| | | | | | | domain proteins. |
| 1266 | BL00421A | 11.79 | 3.864e−20 | 8-26 | BL00421 | Transmembrane 4 family |
| | | | | | | proteins. |
| 1266 | BL00421B | 17.62 | 5.737e−39 | 62-100 | BL00421 | Transmembrane 4 family |
| | | | | | | proteins. |
| 1266 | BL00421C | 12.89 | 3.667e−11 | 146-157 | BL00421 | Transmembrane 4 family |
| | | | | | | proteins. |
| 1266 | BL00421E | 20.97 | 4.115e−26 | 192-221 | BL00421 | Transmembrane 4 family |
| | | | | | | proteins. |
| 1266 | BL00456C | 24.55 | 7.223e−09 | 52-106 | BL00456 | Sodium: solute symporter |
| | | | | | | family proteins. |
| 1266 | PD02680A | 23.51 | 9.341e−10 | 60-110 | PD02680 | TRANSPORT GLUCONATE |
| | | | | | | TRANSMEMBRANE PROTEIN |
| | | | | | | PERMEA. |
| 1266 | PR00259A | 9.27 | 2.241e−22 | 12-35 | PR00259 | TRANSMEMBRANE FOUR |
| | | | | | | FAMILY SIGNATURE |
| 1266 | PR00259B | 14.81 | 8.071e−28 | 56-82 | PR00259 | TRANSMEMBRANE FOUR |
| | | | | | | FAMILY SIGNATURE |
| 1266 | PR00259C | 16.40 | 2.688e−30 | 83-111 | PR00259 | TRANSMEMBRANE FOUR |
| | | | | | | FAMILY SIGNATURE |
| 1266 | PR00259D | 13.50 | 4.316e−26 | 195-221 | PR00259 | TRANSMEMBRANE FOUR |
| | | | | | | FAMILY SIGNATURE |
| 1268 | BL00223A | 15.59 | 4.783e−14 | 252-285 | BL00223 | Annexins repeat |
| | | | | | | proteins domain |
| | | | | | | proteins. |
| 1268 | BL00223A | 15.59 | 5.500e−21 | 93-126 | BL00223 | Annexins repeat |
| | | | | | | proteins domain |
| | | | | | | proteins. |
| 1268 | BL00223A | 15.59 | 5.500e−32 | 21-54 | BL00223 | Annexins repeat |
| | | | | | | proteins domain |
| | | | | | | proteins. |
| 1268 | BL00223B | 28.47 | 1.000e−40 | 162-211 | BL00223 | Annexins repeat |
| | | | | | | proteins domain |
| | | | | | | proteins. |
| 1268 | BL00223C | 24.79 | 1.000e−40 | 239-293 | BL00223 | Annexins repeat |
| | | | | | | proteins domain |
| | | | | | | proteins. |
| 1268 | BL00223C | 24.79 | 1.424e−14 | 80-134 | BL00223 | Annexins repeat |
| | | | | | | proteins domain |
| | | | | | | proteins. |
| 1268 | BL00223C | 24.79 | 2.515e−10 | 8-62 | BL00223 | Annexins repeat |
| | | | | | | proteins domain |
| | | | | | | proteins. |
| 1268 | PR00196A | 11.16 | 6.704e−10 | 102-124 | PR00196 | ANNEXIN FAMILY |
| | | | | | | SIGNATURE |
| 1268 | PR00196A | 11.16 | 9.100e−21 | 30-52 | PR00196 | ANNEXIN FAMILY |
| | | | | | | SIGNATURE |
| 1268 | PR00196B | 10.68 | 3.700e−17 | 70-86 | PR00196 | ANNEXIN FAMILY |
| | | | | | | SIGNATURE |
| 1268 | PR00196C | 10.36 | 2.174e−23 | 97-118 | PR00196 | ANNEXIN FAMILY |
| | | | | | | SIGNATURE |
| 1268 | PR00196C | 10.36 | 5.167e−15 | 25-46 | PR00196 | ANNEXIN FAMILY |
| | | | | | | SIGNATURE |
| 1268 | PR00196C | 10.36 | 7.344e−13 | 256-277 | PR00196 | ANNEXIN FAMILY |
| | | | | | | SIGNATURE |
| 1268 | PR00196D | 21.86 | 3.032e−24 | 180-206 | PR00196 | ANNEXIN FAMILY |
| | | | | | | SIGNATURE |
| 1268 | PR00196E | 9.19 | 1.118e−10 | 101-121 | PR00196 | ANNEXIN FAMILY |
| | | | | | | SIGNATURE |
| 1268 | PR00196E | 9.19 | 8.333e−23 | 260-280 | PR00196 | ANNEXIN FAMILY |
| | | | | | | SIGNATURE |
| 1268 | PR00196F | 13.89 | 2.714e−15 | 288-303 | PR00196 | ANNEXIN FAMILY |
| | | | | | | SIGNATURE |
| 1268 | PR00196F | 13.89 | 4.188e−09 | 129-144 | PR00196 | ANNEXIN FAMILY |
| | | | | | | SIGNATURE |
| 1268 | PR00196G | 11.72 | 3.000e−14 | 304-317 | PR00196 | ANNEXIN FAMILY |
| | | | | | | SIGNATURE |
| 1268 | PR00196G | 11.72 | 9.217e−10 | 229-242 | PR00196 | ANNEXIN FAMILY |
| | | | | | | SIGNATURE |
| 1268 | PR00197B | 7.56 | 2.206e−09 | 30-52 | PR00197 | ANNEXIN TYPE I |
| 1268 | PR00197D | 7.50 | 1.250e−15 | 25-46 | PR00197 | ANNEXIN TYPE I |
| | | | | | | SIGNATURE |
| 1268 | PR00197D | 7.50 | 1.542e−12 | 256-277 | PR00197 | ANNEXIN TYPE I |
| | | | | | | SIGNATURE |
| 1268 | PR00197D | 7.50 | 1.844e−18 | 97-118 | PR00197 | ANNEXIN TYPE I |
| | | | | | | SIGNATURE |
| 1268 | PR00197E | 11.89 | 8.463e−14 | 180-206 | PR00197 | ANNEXIN TYPE I |
| | | | | | | SIGNATURE |
| 1268 | PR00197F | 9.03 | 5.250e−16 | 260-280 | PR00197 | ANNEXIN TYPE I |
| | | | | | | SIGNATURE |
| 1268 | PR00197F | 9.03 | 7.894e−10 | 101-121 | PR00197 | ANNEXIN TYPE I |
| | | | | | | SIGNATURE |
| 1268 | PR00198B | 8.71 | 9.357e−10 | 30-52 | PR00198 | ANNEXIN TYPE II |
| | | | | | | SIGNATURE |
| 1268 | PR00198C | 14.32 | 6.718e−10 | 70-86 | PR00198 | ANNEXIN TYPE II |
| | | | | | | SIGNATURE |
| 1268 | PR00198D | 7.65 | 2.271e−13 | 256-277 | PR00198 | ANNEXIN TYPE II |
| | | | | | | SIGNATURE |
| 1268 | PR00198D | 7.65 | 8.333e−15 | 97-118 | PR00198 | ANNEXIN TYPE II |
| | | | | | | SIGNATURE |
| 1268 | PR00198D | 7.65 | 9.894e−13 | 25-46 | PR00198 | ANNEXIN TYPE II |
| | | | | | | SIGNATURE |
| 1268 | PR00198E | 14.67 | 6.381e−11 | 180-206 | PR00198 | ANNEXIN TYPE II |
| | | | | | | SIGNATURE |
| 1268 | PR00198G | 8.09 | 4.656e−09 | 101-121 | PR00198 | ANNEXIN TYPE II |
| | | | | | | SIGNATURE |
| 1268 | PR00198G | 8.09 | 7.943e−16 | 260-280 | PR00198 | ANNEXIN TYPE II |
| | | | | | | SIGNATURE |
| 1268 | PR00198H | 12.05 | 1.462e−10 | 304-317 | PR00198 | ANNEXIN TYPE II |
| | | | | | | SIGNATURE |
| 1268 | PR00199B | 6.86 | 1.574e−13 | 30-52 | PR00199 | ANNEXIN TYPE III |
| | | | | | | SIGNATURE |
| 1268 | PR00199B | 6.86 | 8.313e−09 | 102-124 | PR00199 | ANNEXIN TYPE III |
| | | | | | | SIGNATURE |
| 1268 | PR00199C | 13.84 | 1.857e−09 | 70-86 | PR00199 | ANNEXIN TYPE III |
| | | | | | | SIGNATURE |
| 1268 | PR00199D | 5.65 | 3.443e−15 | 97-118 | PR00199 | ANNEXIN TYPE III |
| | | | | | | SIGNATURE |
| 1268 | PR00199D | 5.65 | 5.375e−14 | 25-46 | PR00199 | ANNEXIN TYPE III |
| | | | | | | SIGNATURE |
| 1268 | PR00199D | 5.65 | 7.987e−13 | 256-277 | PR00199 | ANNEXIN TYPE III |
| | | | | | | SIGNATURE |
| 1268 | PR00199F | 16.19 | 5.636e−16 | 180-206 | PR00199 | ANNEXIN TYPE III |
| | | | | | | SIGNATURE |
| 1268 | PR00199G | 9.09 | 6.365e−09 | 102-127 | PR00199 | ANNEXIN TYPE III |
| | | | | | | SIGNATURE |
| 1268 | PR00199G | 9.09 | 8.364e−21 | 261-286 | PR00199 | ANNEXIN TYPE III |
| | | | | | | SIGNATURE |
| 1268 | PR00199H | 12.62 | 5.339e−12 | 304-317 | PR00199 | ANNEXIN TYPE III |
| | | | | | | SIGNATURE |
| 1268 | PR00200A | 4.93 | 2.125e−14 | 5-15 | PR00200 | ANNEXIN TYPE IV |
| | | | | | | SIGNATURE |
| 1268 | PR00200B | 7.39 | 1.643e−29 | 30-52 | PR00200 | ANNEXIN TYPE IV |
| | | | | | | SIGNATURE |
| 1268 | PR00200C | 8.76 | 1.500e−12 | 54-62 | PR00200 | ANNEXIN TYPE IV |
| | | | | | | SIGNATURE |
| 1268 | PR00200D | 10.01 | 2.286e−22 | 70-86 | PR00200 | ANNEXIN TYPE IV |
| | | | | | | SIGNATURE |
| 1268 | PR00200E | 10.00 | 1.110e−27 | 97-118 | PR00200 | ANNEXIN TYPE IV |
| | | | | | | SIGNATURE |
| 1268 | PR00200E | 10.00 | 2.111e−14 | 25-46 | PR00200 | ANNEXIN TYPE IV |
| | | | | | | SIGNATURE |
| 1268 | PR00200E | 10.00 | 2.859e−11 | 256-277 | PR00200 | ANNEXIN TYPE IV |
| | | | | | | SIGNATURE |
| 1268 | PR00200F | 13.72 | 1.118e−35 | 180-206 | PR00200 | ANNEXIN TYPE IV |
| | | | | | | SIGNATURE |
| 1268 | PR00200G | 9.43 | 1.000e−34 | 260-286 | PR00200 | ANNEXIN TYPE IV |
| | | | | | | SIGNATURE |
| 1268 | PR00200G | 9.43 | 1.983e−09 | 101-127 | PR00200 | ANNEXIN TYPE IV |
| | | | | | | SIGNATURE |
| 1268 | PR00200G | 9.43 | 5.294e−11 | 29-55 | PR00200 | ANNEXIN TYPE IV |
| | | | | | | SIGNATURE |
| 1268 | PR00200H | 13.68 | 1.766e−18 | 304-317 | PR00200 | ANNEXIN TYPE IV |
| | | | | | | SIGNATURE |
| 1268 | PR00201A | 6.05 | 4.770e−16 | 30-52 | PR00201 | ANNEXIN TYPE V |
| | | | | | | SIGNATURE |
| 1268 | PR00201C | 11.13 | 9.316e−12 | 70-86 | PR00201 | ANNEXIN TYPE V |
| | | | | | | SIGNATURE |
| 1268 | PR00201D | 10.49 | 1.456e−15 | 97-118 | PR00201 | ANNEXIN TYPE V |
| | | | | | | SIGNATURE |
| 1268 | PR00201D | 10.49 | 6.179e−09 | 25-46 | PR00201 | ANNEXIN TYPE V |
| | | | | | | SIGNATURE |
| 1268 | PR00201E | 12.37 | 4.103e−15 | 180-206 | PR00201 | ANNEXIN TYPE V |
| | | | | | | SIGNATURE |
| 1268 | PR00201G | 11.02 | 8.402e−10 | 29-55 | PR00201 | ANNEXIN TYPE V |
| | | | | | | SIGNATURE |
| 1268 | PR00201G | 11.02 | 9.419e−26 | 260-286 | PR00201 | ANNEXIN TYPE V |
| | | | | | | SIGNATURE |
| 1268 | PR00201G | 11.02 | 9.912e−11 | 101-127 | PR00201 | ANNEXIN TYPE V |
| | | | | | | SIGNATURE |
| 1268 | PR00201H | 12.04 | 4.375e−14 | 304-317 | PR00201 | ANNEXIN TYPE V |
| | | | | | | SIGNATURE |
| 1268 | PR00202B | 11.44 | 2.763e−11 | 29-52 | PR00202 | ANNEXIN TYPE VI |
| | | | | | | SIGNATURE |
| 1268 | PR00202C | 13.34 | 3.800e−11 | 70-86 | PR00202 | ANNEXIN TYPE VI |
| | | | | | | SIGNATURE |
| 1268 | PR00202D | 5.58 | 1.908e−09 | 25-46 | PR00202 | ANNEXIN TYPE VI |
| | | | | | | SIGNATURE |
| 1268 | PR00202D | 5.58 | 3.224e−15 | 97-118 | PR00202 | ANNEXIN TYPE VI |
| | | | | | | SIGNATURE |
| 1268 | PR00202E | 13.00 | 4.643e−16 | 180-206 | PR00202 | ANNEXIN TYPE VI |
| | | | | | | SIGNATURE |
| 1268 | PR00202G | 8.01 | 4.833e−28 | 260-286 | PR00202 | ANNEXIN TYPE VI |
| | | | | | | SIGNATURE |
| 1268 | PR00202G | 8.01 | 5.881e−09 | 101-127 | PR00202 | ANNEXIN TYPE VI |
| | | | | | | SIGNATURE |
| 1268 | PR00202G | 8.01 | 9.237e−09 | 29-55 | PR00202 | ANNEXIN TYPE VI |
| | | | | | | SIGNATURE |
| 1268 | PR00202H | 9.20 | 4.740e−11 | 304-317 | PR00202 | ANNEXIN TYPE VI |
| | | | | | | SIGNATURE |
| 1268 | PR00301D | 15.51 | 7.395e−09 | 38-58 | PR00301 | 70 KD HEAT SHOCK |
| | | | | | | PROTEIN SIGNATURE |
| 1269 | BL01179A | 12.63 | 1.429e−11 | 209-220 | BL01179 | Phosphotyrosine |
| | | | | | | interaction domain |
| | | | | | | proteins (PID) profile. |
| 1269 | BL01179B | 15.18 | 9.679e−11 | 400-414 | BL01179 | Phosphotyrosine |
| | | | | | | interaction domain |
| | | | | | | proteins (PID) profile. |
| 1270 | BL01033A | 16.94 | 7.923e−18 | 25-46 | BL01033 | Globins profile. |
| 1270 | BL01033B | 13.81 | 1.000e−15 | 93-104 | BL01033 | Globins profile. |
| 1270 | PR00612D | 9.76 | 3.415e−10 | 80-93 | PR00612 | ALPHA HAEMOGLOBIN |
| | | | | | | SIGNATURE |
| 1270 | PR00814A | 12.94 | 5.500e−22 | 30-46 | PR00814 | BETA HAEMOGLOBIN |
| | | | | | | SIGNATURE |
| 1270 | PR00814B | 9.18 | 3.250e−17 | 48-63 | PR00814 | BETA HAEMOGLOBIN |
| | | | | | | SIGNATURE |
| 1270 | PR00814C | 9.20 | 4.273e−16 | 72-89 | PR00814 | BETA HAEMOGLOBIN |
| | | | | | | SIGNATURE |
| 1270 | PR00814D | 15.25 | 2.286e−09 | 94-99 | PR00814 | BETA HAEMOGLOBIN |
| | | | | | | SIGNATURE |
| 1270 | PR00814E | 10.17 | 8.071e−19 | 128-144 | PR00814 | BETA HAEMOGLOBIN |
| | | | | | | SIGNATURE |
| 1273 | BL00407A | 18.57 | 8.500e−35 | 11-47 | BL00407 | Connexins proteins. |
| 1273 | BL00407B | 14.23 | 1.000e−35 | 48-78 | BL00407 | Connexins proteins. |
| 1273 | BL00407C | 14.61 | 6.850e−31 | 79-106 | BL00407 | Connexins proteins. |
| 1273 | BL00407D | 17.61 | 5.091e−28 | 157-186 | BL00407 | Connexins proteins. |
| 1273 | BL00407E | 22.17 | 1.000e−40 | 195-239 | BL00407 | Connexins proteins. |
| 1273 | PR00206A | 11.35 | 9.053e−27 | 29-53 | PR00206 | CONNEXIN SIGNATURE |
| 1273 | PR00206B | 13.75 | 7.882e−30 | 60-82 | PR00206 | CONNEXIN SIGNATURE |
| 1273 | PR00206C | 15.16 | 3.842e−23 | 85-105 | PR00206 | CONNEXIN SIGNATURE |
| 1273 | PR00206D | 16.57 | 4.429e−26 | 159-185 | PR00206 | CONNEXIN SIGNATURE |
| 1273 | PR00206E | 15.09 | 1.857e−25 | 195-215 | PR00206 | CONNEXIN SIGNATURE |
| 1273 | PR00206F | 16.77 | 6.571e−23 | 216-239 | PR00206 | CONNEXIN SIGNATURE |
| 1274 | BL00021B | 13.33 | 3.786e−18 | 34-51 | BL00021 | Kringle domain |
| | | | | | | proteins. |
| 1274 | BL00021D | 24.56 | 4.759e−11 | 190-231 | BL00021 | Kringle domain |
| | | | | | | proteins. |
| 1274 | BL00134A | 11.96 | 9.609e−18 | 34-50 | BL00134 | Serine proteases, |
| | | | | | | trypsin family, |
| | | | | | | histidine proteins. |
| 1274 | BL00134B | 15.99 | 5.714e−19 | 185-208 | BL00134 | Serine proteases, |
| | | | | | | trypsin family, |
| | | | | | | histidine proteins. |
| 1274 | BL00134C | 13.45 | 4.750e−12 | 218-231 | BL00134 | Serine proteases, |
| | | | | | | trypsin family, |
| | | | | | | histidine proteins. |
| 1274 | BL00495K | 12.58 | 3.796e−09 | 36-68 | BL00495 | Apple domain proteins. |
| 1274 | BL01253D | 4.84 | 9.855e−12 | 34-47 | BL01253 | Type I fibronectin |
| | | | | | | domain proteins. |
| 1274 | BL01253G | 11.34 | 7.972e−14 | 184-197 | BL01253 | Type I fibronectin |
| | | | | | | domain proteins. |
| 1274 | BL01253H | 13.15 | 7.680e−12 | 200-234 | BL01253 | Type I fibronectin |
| | | | | | | domain proteins. |
| 1274 | PR00722A | 12.27 | 6.087e−16 | 35-50 | PR00722 | CHYMOTRYPSIN SERINE |
| | | | | | | PROTEASE FAMILY (S1) |
| | | | | | | SIGNATURE |
| 1274 | PR00722C | 10.87 | 5.500e−15 | 184-196 | PR00722 | CHYMOTRYPSIN SERINE |
| | | | | | | PROTEASE FAMILY (S1) |
| | | | | | | SIGNATURE |
| 1276 | PD01876C | 21.73 | 8.529e−11 | 461-513 | PD01876 | ANTIGEN MELANOMA- |
| | | | | | | ASSOCIATED MULTIGENE |
| | | | | | | FAMILY TUM. |
| 1276 | PR00411A | 15.95 | 4.724e−09 | 749-771 | PR00411 | PYRIDINE NUCLEOTIDE |
| | | | | | | DISULPHIDE REDUCTASE |
| | | | | | | CLASS-I SIGNATURE |
| 1279 | BL01310 | 14.74 | 1.321e−36 | 28-63 | BL01310 | ATP1G1/PLM/MAT8 |
| | | | | | | family proteins. |
| 1281 | BL00296B | 15.98 | 6.625e−09 | 83-136 | BL00296 | Chaperonins cpn60 |
| | | | | | | proteins. |
| 1281 | BL00750A | 20.07 | 1.310e−30 | 32-74 | BL00750 | Chaperonins TCP-1 |
| | | | | | | proteins. |
| 1281 | BL00750B | 16.17 | 7.517e−37 | 76-125 | BL00750 | Chaperonins TCP-1 |
| | | | | | | proteins. |
| 1281 | BL00750C | 25.65 | 2.909e−15 | 157-188 | BL00750 | Chaperonins TCP-1 |
| | | | | | | proteins. |
| 1281 | BL00750D | 16.16 | 9.719e−11 | 206-224 | BL00750 | Chaperonins TCP-1 |
| | | | | | | proteins. |
| 1281 | BL00750E | 24.59 | 2.406e−21 | 296-332 | BL00750 | Chaperonins TCP-1 |
| | | | | | | proteins. |
| 1281 | BL00750F | 18.40 | 4.938e−25 | 371-411 | BL00750 | Chaperonins TCP-1 |
| | | | | | | proteins. |
| 1281 | BL00750G | 20.12 | 2.324e−28 | 432-471 | BL00750 | Chaperonins TCP-1 |
| | | | | | | proteins. |
| 1281 | BL00750H | 21.44 | 2.875e−25 | 491-525 | BL00750 | Chaperonins TCP-1 |
| | | | | | | proteins. |
| 1281 | PR00304A | 9.20 | 5.737e−15 | 41-57 | PR00304 | TAILLESS COMPLEX |
| | | | | | | POLYPEPTIDE 1 |
| | | | | | | (CHAPERONE) SIGNATURE |
| 1281 | PR00304B | 11.60 | 9.700e−18 | 63-81 | PR00304 | TAILLESS COMPLEX |
| | | | | | | POLYPEPTIDE 1 |
| | | | | | | (CHAPERONE) SIGNATURE |
| 1281 | PR00304C | 8.69 | 2.575e−17 | 93-112 | PR00304 | TAILLESS COMPLEX |
| | | | | | | POLYPEPTIDE 1 |
| | | | | | | (CHAPERONE) SIGNATURE |
| 1281 | PR00304D | 11.04 | 4.405e−17 | 374-396 | PR00304 | TAILLESS COMPLEX |
| | | | | | | POLYPEPTIDE 1 |
| | | | | | | (CHAPERONE) SIGNATURE |
| 1281 | PR00304E | 7.79 | 9.217e−13 | 408-420 | PR00304 | TAILLESS COMPLEX |
| | | | | | | POLYPEPTIDE 1 |
| | | | | | | (CHAPERONE) SIGNATURE |
| 1282 | BL00194 | 12.16 | 3.813e−15 | 47-59 | BL00194 | Thioredoxin family |
| | | | | | | proteins. |
| 1282 | BL00194 | 12.16 | 4.857e−16 | 182-194 | BL00194 | Thioredoxin family |
| | | | | | | proteins. |
| 1282 | PR00421A | 10.15 | 4.000e−10 | 46-54 | PR00421 | THIOREDOXIN FAMILY |
| | | | | | | SIGNATURE |
| 1282 | PR00421A | 10.15 | 4.857e−10 | 181-189 | PR00421 | THIOREDOXIN FAMILY |
| | | | | | | SIGNATURE |
| 1282 | PR00421B | 11.40 | 3.348e−12 | 189-198 | PR00421 | THIOREDOXIN FAMILY |
| | | | | | | SIGNATURE |
| 1282 | PR00421B | 11.40 | 9.379e−11 | 54-63 | PR00421 | THIOREDOXIN FAMILY |
| | | | | | | SIGNATURE |
| 1282 | PR00421C | 13.60 | 5.714e−10 | 233-244 | PR00421 | THIOREDOXIN FAMILY |
| | | | | | | SIGNATURE |
| 1283 | BL00142 | 8.38 | 6.625e−10 | 559-569 | BL00142 | Neutral zinc |
| | | | | | | metallopeptidases, |
| | | | | | | zinc-binding region |
| | | | | | | proteins. |
| 1283 | PF00084B | 9.45 | 5.500e−09 | 1370-1381 | PF00084 | Sushi domain proteins |
| | | | | | | (SCR repeat proteins. |
| 1283 | PR00014A | 8.22 | 8.773e−09 | 687-696 | PR00014 | FIBRONECTIN TYPE III |
| | | | | | | REPEAT SIGNATURE |
| 1283 | PR00480B | 15.41 | 2.286e−09 | 554-572 | PR00480 | ASTACIN FAMILY |
| | | | | | | SIGNATURE |
| 1284 | BL00299 | 28.84 | 4.188e−19 | 92-143 | BL00299 | Ubiquitin domain |
| | | | | | | proteins. |
| 1285 | BL00036 | 9.02 | 4.214e−13 | 197-209 | BL00036 | bZIP transcription |
| | | | | | | factors basic domain |
| | | | | | | proteins. |
| 1285 | PR00042A | 10.04 | 2.500e−22 | 113-130 | PR00042 | FOS TRANSFORMING |
| | | | | | | PROTEIN SIGNATURE |
| 1285 | PR00042B | 10.70 | 1.191e−21 | 181-197 | PR00042 | FOS TRANSFORMING |
| | | | | | | PROTEIN SIGNATURE |
| 1285 | PR00042C | 8.29 | 1.273e−22 | 198-214 | PR00042 | FOS TRANSFORMING |
| | | | | | | PROTEIN SIGNATURE |
| 1285 | PR00042D | 8.97 | 2.500e−27 | 216-237 | PR00042 | FOS TRANSFORMING |
| | | | | | | PROTEIN SIGNATURE |
| 1285 | PR00042E | 9.69 | 8.200e−29 | 237-260 | PR00042 | FOS TRANSFORMING |
| | | | | | | PROTEIN SIGNATURE |
| 1285 | PR00043B | 8.73 | 5.652e−09 | 197-213 | PR00043 | JUN TRANSCRIPTION |
| | | | | | | FACTOR SIGNATURE |
| 1286 | BL00297A | 17.58 | 4.375e−37 | 31-67 | BL00297 | Heat shock hsp70 |
| | | | | | | proteins family |
| | | | | | | proteins. |
| 1286 | BL00297B | 22.91 | 4.316e−38 | 71-119 | BL00297 | Heat shock hsp70 |
| | | | | | | proteins family |
| | | | | | | proteins. |
| 1286 | BL00297C | 9.51 | 1.000e−40 | 160-211 | BL00297 | Heat shock hsp70 |
| | | | | | | proteins family |
| | | | | | | proteins. |
| 1286 | BL00297D | 11.95 | 1.000e−40 | 220-259 | BL00297 | Heat shock hsp70 |
| | | | | | | proteins family |
| | | | | | | proteins. |
| 1286 | BL00297E | 18.56 | 6.400e−35 | 316-359 | BL00297 | Heat shock hsp70 |
| | | | | | | proteins family |
| | | | | | | proteins |
| 1286 | BL00297F | 11.84 | 1.692e−30 | 380-407 | BL00297 | Heat shock hsp70 |
| | | | | | | proteins family |
| | | | | | | proteins. |
| 1286 | BL00297G | 21.87 | 1.000e−40 | 412-466 | BL00297 | Heat shock hsp70 |
| | | | | | | proteins family |
| | | | | | | proteins. |
| 1286 | BL00297H | 15.46 | 1.000e−40 | 475-528 | BL00297 | Heat shock hsp70 |
| | | | | | | proteins family |
| | | | | | | proteins. |
| 1286 | PR00301A | 14.84 | 5.846e−17 | 29-42 | PR00301 | 70 KD HEAT SHOCK |
| | | | | | | PROTEIN SIGNATURE |
| 1286 | PR00301B | 15.36 | 3.118e−16 | 57-69 | PR00301 | 70 KD HEAT SHOCK |
| | | | | | | PROTEIN SIGNATURE |
| 1286 | PR00301D | 15.51 | 2.227e−26 | 168-188 | PR00301 | 70 KD HEAT SHOCK |
| | | | | | | PROTEIN SIGNATURE |
| 1286 | PR00301E | 8.11 | 4.789e−13 | 228-238 | PR00301 | 70 KD HEAT SHOCK |
| | | | | | | PROTEIN SIGNATURE |
| 1286 | PR00301F | 13.98 | 1.857e−19 | 355-371 | PR00301 | 70 KD HEAT SHOCK |
| | | | | | | PROTEIN SIGNATURE |
| 1286 | PR00301G | 13.78 | 1.000e−25 | 387-407 | PR00301 | 70 KD HEAT SHOCK |
| | | | | | | PROTEIN SIGNATURE |
| 1286 | PR00301H | 8.81 | 5.909e−20 | 412-431 | PR00301 | 70 KD HEAT SHOCK |
| | | | | | | PROTEIN SIGNATURE |
| 1286 | PR00301I | 12.76 | 5.500e−18 | 493-509 | PR00301 | 70 KD HEAT SHOCK |
| | | | | | | PROTEIN SIGNATURE |
| 1287 | BL00194 | 12.16 | 3.813e−15 | 398-410 | BL00194 | Thioredoxin family |
| | | | | | | proteins. |
| 1287 | BL00194 | 12.16 | 5.091e−17 | 49-61 | BL00194 | Thioredoxin family |
| | | | | | | proteins. |
| 1287 | PR00421A | 10.15 | 1.429e−10 | 397-405 | PR00421 | THIOREDOXIN FAMILY |
| | | | | | | SIGNATURE |
| 1287 | PR00421A | 10.15 | 3.160e−09 | 48-56 | PR00421 | THIOREDOXIN FAMILY |
| | | | | | | SIGNATURE |
| 1287 | PR00421B | 11.40 | 1.000e−13 | 56-65 | PR00421 | THIOREDOXIN FAMILY |
| | | | | | | SIGNATURE |
| 1287 | PR00421B | 11.40 | 3.348e−12 | 405-414 | PR00421 | THIOREDOXIN FAMILY |
| | | | | | | SIGNATURE |
| 1287 | PR00421C | 13.60 | 5.286e−10 | 446-457 | PR00421 | THIOREDOXIN FAMILY |
| | | | | | | SIGNATURE |
| 1287 | PR00421C | 13.60 | 5.500e−09 | 96-107 | PR00421 | THIOREDOXIN FAMILY |
| | | | | | | SIGNATURE |
| 1288 | BL00251A | 14.27 | 1.383e−10 | 121-138 | BL00251 | TNF family proteins. |
| 1288 | BL00251B | 11.40 | 5.200e−17 | 171-190 | BL00251 | TNF family proteins. |
| 1288 | BL00251C | 13.97 | 5.250e−14 | 238-256 | BL00251 | TNF family proteins. |
| 1288 | BL00251D | 12.03 | 1.529e−09 | 269-280 | BL00251 | TNF family proteins. |
| 1289 | BL00387A | 14.69 | 3.423e−14 | 44-58 | BL00387 | Inorganic |
| | | | | | | pyrophosphatase |
| | | | | | | proteins. |
| 1289 | BL00387B | 27.02 | 4.103e−31 | 72-109 | BL00387 | Inorganic |
| | | | | | | pyrophosphatase |
| | | | | | | proteins. |
| 1289 | BL00387C | 20.60 | 1.310e−24 | 133-163 | BL00387 | Inorganic |
| | | | | | | pyrophosphatase |
| | | | | | | proteins. |
| 1289 | BL00387D | 21.31 | 6.786e−23 | 167-199 | BL00387 | Inorganic |
| | | | | | | pyrophosphatase |
| | | | | | | proteins. |
| 1290 | BL00115Z | 3.12 | 7.022e−09 | 255-303 | BL00115 | Eukaryotic RNA |
| | | | | | | polymerase II |
| | | | | | | heptapeptide repeat |
| | | | | | | proteins. |
| 1290 | BL00478B | 14.79 | 3.500e−12 | 405-419 | BL00478 | LIM domain proteins. |
| 1290 | BL00478B | 14.79 | 3.739e−14 | 463-477 | BL00478 | LIM domain proteins. |
| 1290 | BL00478B | 14.79 | 6.000e−12 | 530-544 | BL00478 | LIM domain proteins. |
| 1290 | DM00984B | 15.18 | 4.822e−25 | 420-474 | DM00984 | w MYOD MYOBLAST |
| | | | | | | DETERMINATION SHORT. |
| 1290 | DM00984C | 7.66 | 8.036e−11 | 475-488 | DM00984 | w MYOD MYOBLAST |
| | | | | | | DETERMINATION SHORT. |
| 1292 | BL00470A | 16.25 | 6.077e−11 | 57-77 | BL00470 | Isocitrate and |
| | | | | | | isopropylmalate |
| | | | | | | dehydrogenases |
| | | | | | | proteins. |
| 1292 | BL00470C | 15.43 | 4.375e−11 | 162-176 | BL00470 | Isocitrate and |
| | | | | | | isopropylmalate |
| | | | | | | dehydrogenases |
| | | | | | | proteins. |
| 1292 | BL00470D | 21.75 | 8.500e−13 | 269-299 | BL00470 | Isocitrate and |
| | | | | | | isopropylmalate |
| | | | | | | dehydrogenases |
| | | | | | | proteins. |
| 1293 | BL00325A | 24.83 | 2.125e−26 | 61-92 | BL00325 | Actin-depolymerizing |
| | | | | | | proteins. |
| 1293 | BL00325B | 21.66 | 1.000e−40 | 93-138 | BL00325 | Actin-depolymerizing |
| | | | | | | proteins. |
| 1293 | PR00006A | 13.46 | 2.000e−31 | 17-41 | PR00006 | COFILIN/DESTRIN FAMILY |
| | | | | | | SIGNATURE |
| 1293 | PR00006B | 4.38 | 1.391e−27 | 79-99 | PR00006 | COFILIN/DESTRIN FAMILY |
| | | | | | | SIGNATURE |
| 1293 | PR00006C | 9.30 | 1.500e−28 | 100-121 | PR00006 | COFILIN/DESTRIN FAMILY |
| | | | | | | SIGNATURE |
| 1293 | PR00006D | 14.99 | 9.750e−21 | 122-138 | PR00006 | COFILIN/DESTRIN FAMILY |
| | | | | | | SIGNATURE |
| 1293 | PR00006E | 9.61 | 1.462e−14 | 153-165 | PR00006 | COFILIN/DESTRIN FAMILY |
| | | | | | | SIGNATURE |
| 1294 | BL00183 | 28.97 | 6.586e−33 | 50-97 | BL00183 | Ubiquitin-conjugating |
| | | | | | | enzymes proteins. |
| 1295 | BL00113A | 12.74 | 7.508e−09 | 8-24 | BL00113 | Adenylate kinase |
| | | | | | | proteins. |
| 1295 | BL00211A | 12.23 | 1.000e−09 | 9-20 | BL00211 | ABC transporters family |
| | | | | | | proteins. |
| 1295 | BL00567A | 10.66 | 8.714e−09 | 6-24 | BL00567 | Phosphoribulokinase |
| | | | | | | proteins. |
| 1295 | BL00674B | 4.46 | 9.392e−09 | 4-25 | BL00674 | AAA-protein family |
| | | | | | | proteins. |
| 1295 | BL00856A | 17.25 | 4.176e−11 | 8-20 | BL00856 | Guanylate kinase |
| | | | | | | proteins. |
| 1295 | BL00856B | 9.61 | 2.565e−22 | 36-56 | BL00856 | Guanylate kinase |
| | | | | | | proteins. |
| 1295 | BL00856C | 29.21 | 1.409e−38 | 64-111 | BL00856 | Guanylate kinase |
| | | | | | | proteins. |
| 1295 | PR00449A | 13.20 | 3.596e−10 | 5-26 | PR00449 | TRANSFORMING PROTEIN |
| | | | | | | P21 RAS SIGNATURE |
| 1297 | BL00291A | 4.49 | 3.586e−09 | 55-89 | BL00291 | Prion protein. |
| 1297 | BL00309A | 19.16 | 5.065e−16 | 125-149 | BL00309 | Vertebrate galactoside- |
| | | | | | | binding lectin |
| | | | | | | proteins. |
| 1297 | BL00309B | 12.11 | 4.500e−16 | 150-164 | BL00309 | Vertebrate galactoside- |
| | | | | | | binding lectin |
| | | | | | | proteins. |
| 1297 | BL00309C | 18.65 | 1.000e−22 | 171-195 | BL00309 | Vertebrate galactoside- |
| | | | | | | binding lectin |
| | | | | | | proteins. |
| 1297 | BL00604F | 5.96 | 4.709e−09 | 23-67 | BL00604 | Synaptophysin/ |
| | | | | | | synaptoporin proteins. |
| 1297 | DM00250B | 13.84 | 3.593e−12 | 40-63 | DM00250 | kw ANNEXIN ANTIGEN |
| | | | | | | PROLINE TUMOR. |
| 1300 | BL01113A | 17.99 | 5.091e−11 | 94-120 | BL01113 | C1q domain proteins. |
| 1303 | BL00019A | 12.56 | 3.475e−13 | 45-55 | BL00019 | Actinin-type actin- |
| | | | | | | binding domain |
| | | | | | | proteins. |
| 1303 | BL00019B | 13.34 | 8.096e−10 | 70-92 | BL00019 | Actinin-type actin- |
| | | | | | | binding domain |
| | | | | | | proteins. |
| 1303 | BL00019B | 13.34 | 8.548e−16 | 1992-2014 | BL00019 | Actinin-type actin- |
| | | | | | | binding domain |
| | | | | | | proteins. |
| 1303 | BL00019C | 14.66 | 5.171e−32 | 118-153 | BL00019 | Actinin-type actin- |
| | | | | | | binding domain |
| | | | | | | proteins. |
| 1303 | BL00019D | 15.33 | 2.703e−23 | 2455-2484 | BL00019 | Actinin-type actin- |
| | | | | | | binding domain |
| | | | | | | proteins. |
| 1303 | BL00019F | 19.68 | 3.195e−15 | 154-175 | BL00019 | Actinin-type actin- |
| | | | | | | binding domain |
| | | | | | | proteins. |
| 1303 | PR00401C | 13.16 | 1.750e−09 | 1717-1728 | PR00401 | SH2 DOMAIN SIGNATURE |
| 1306 | BL01239 | 16.10 | 1.000e−40 | 17-70 | BL01239 | Dynein light chain type |
| | | | | | | 1 proteins. |
| 1307 | BL00463 | 8.22 | 5.071e−09 | 125-136 | BL00463 | Fungal Zn(2)-Cys(6) |
| | | | | | | binuclear cluster |
| | | | | | | domain proteins. |
| 1309 | BL01207A | 12.21 | 9.069e−18 | 66-81 | BL01207 | Glypicans proteins. |
| 1309 | BL01207B | 23.69 | 3.423e−37 | 193-238 | BL01207 | Glypicans proteins. |
| 1309 | BL01207C | 19.08 | 9.308e−39 | 252-287 | BL01207 | Glypicans proteins. |
| 1309 | BL01207D | 23.23 | 5.655e−29 | 423-457 | BL01207 | Glypicans proteins. |
| 1309 | BL01207E | 13.70 | 7.375e−16 | 496-512 | BL01207 | Glypicans proteins. |
| 1310 | BL00142 | 8.38 | 1.857e−09 | 210-220 | BL00142 | Neutral zinc |
| | | | | | | metallopeptidases, |
| | | | | | | zinc-binding region |
| | | | | | | proteins. |
| 1310 | BL01177C | 17.39 | 5.333e−11 | 712-730 | BL01177 | Anaphylatoxin domain |
| | | | | | | proteins. |
| 1310 | BL01180A | 15.41 | 4.130e−11 | 220-231 | BL01180 | CUB domain proteins |
| | | | | | | profile. |
| 1310 | BL01180B | 11.58 | 3.647e−11 | 488-498 | BL01180 | CUB domain proteins |
| | | | | | | profile. |
| 1310 | BL01180B | 11.58 | 4.000e−09 | 644-654 | BL01180 | CUB domain proteins |
| | | | | | | profile. |
| 1310 | BL01180B | 11.58 | 4.750e−09 | 800-810 | BL01180 | CUB domain proteins |
| | | | | | | profile. |
| 1310 | BL01180B | 11.58 | 6.625e−09 | 917-927 | BL01180 | CUB domain proteins |
| | | | | | | profile. |
| 1310 | BL01180B | 11.58 | 8.364e−13 | 375-385 | BL01180 | CUB domain proteins |
| | | | | | | profile. |
| 1310 | BL01180C | 17.69 | 1.000e−10 | 394-407 | BL01180 | CUB domain proteins |
| | | | | | | profile. |
| 1310 | BL01180C | 17.69 | 4.500e−09 | 936-949 | BL01180 | CUB domain proteins |
| | | | | | | profile. |
| 1310 | BL01180C | 17.69 | 8.000e−09 | 663-676 | BL01180 | CUB domain proteins |
| | | | | | | profile. |
| 1310 | BL01187B | 12.04 | 2.174e−13 | 718-733 | BL01187 | Calcium-binding EGF- |
| | | | | | | like domain proteins |
| | | | | | | pattern proteins. |
| 1310 | BL01187B | 12.04 | 6.250e−17 | 563-578 | BL01187 | Calcium-binding EGF- |
| | | | | | | like domain proteins |
| | | | | | | pattern proteins. |
| 1310 | PR00010C | 11.16 | 4.273e−10 | 723-733 | PR00010 | TYPE II EGF-LIKE |
| | | | | | | SIGNATURE |
| 1310 | PR00480A | 19.40 | 3.250e−17 | 151-169 | PR00480 | ASTACIN FAMILY |
| | | | | | | SIGNATURE |
| 1310 | PR00480B | 15.41 | 9.143e−21 | 205-223 | PR00480 | ASTACIN FAMILY |
| | | | | | | SIGNATURE |
| 1310 | PR00480C | 12.53 | 1.300e−17 | 224-241 | PR00480 | ASTACIN FAMILY |
| | | | | | | SIGNATURE |
| 1310 | PR00480D | 14.26 | 7.545e−17 | 263-278 | PR00480 | ASTACIN FAMILY |
| | | | | | | SIGNATURE |
| 1310 | PR00480E | 14.96 | 6.727e−12 | 305-318 | PR00480 | ASTACIN FAMILY |
| | | | | | | SIGNATURE |
| 1311 | BL00892A | 18.17 | 8.412e−30 | 27-57 | BL00892 | HIT family proteins. |
| 1311 | BL00892B | 16.86 | 1.964e−22 | 93-116 | BL00892 | HIT family proteins. |
| 1311 | PR00332A | 10.15 | 9.625e−18 | 18-34 | PR00332 | HISTIDINE TRIAD FAMILY |
| | | | | | | SIGNATURE |
| 1311 | PR00332B | 13.62 | 1.000e−23 | 39-57 | PR00332 | HISTIDINE TRIAD FAMILY |
| | | | | | | SIGNATURE |
| 1311 | PR00332C | 7.37 | 2.200e−13 | 106-116 | PR00332 | HISTIDINE TRIAD FAMILY |
| | | | | | | SIGNATURE |
| 1312 | BL00854A | 33.93 | 9.036e−29 | 64-109 | BL00854 | Proteasome B-type |
| | | | | | | subunits proteins. |
| 1312 | BL00854C | 29.92 | 1.794e−14 | 171-199 | BL00854 | Proteasome B-type |
| | | | | | | subunits proteins. |
| 1312 | BL00854D | 13.76 | 2.800e−09 | 222-231 | BL00854 | Proteasome B-type |
| | | | | | | subunits proteins. |
| 1312 | PR00141A | 11.36 | 3.613e−17 | 67-82 | PR00141 | PROTEASOME COMPONENT |
| | | | | | | SIGNATURE |
| 1312 | PR00141B | 11.15 | 5.304e−14 | 188-199 | PR00141 | PROTEASOME COMPONENT |
| | | | | | | SIGNATURE |
| 1312 | PR00141C | 11.15 | 3.368e−10 | 199-210 | PR00141 | PROTEASOME COMPONENT |
| | | | | | | SIGNATURE |
| 1312 | PR00141D | 12.45 | 1.923e−12 | 224-235 | PR00141 | PROTEASOME COMPONENT |
| | | | | | | SIGNATURE |
| 1313 | BL00226A | 12.77 | 1.360e−14 | 71-85 | BL00226 | Intermediate filaments |
| | | | | | | proteins. |
| 1313 | BL00226B | 23.86 | 7.545e−17 | 171-218 | BL00226 | Intermediate filaments |
| | | | | | | proteins. |
| 1313 | BL00226C | 13.23 | 5.800e−13 | 236-266 | BL00226 | Intermediate filaments |
| | | | | | | proteins. |
| 1313 | BL00226D | 19.10 | 1.500e−30 | 336-382 | BL00226 | Intermediate filaments |
| | | | | | | proteins. |
| 1314 | DM01753B | 6.47 | 1.247e−09 | 276-285 | DM01753 | 6 kw OSTEOBLAST MAJOR |
| | | | | | | IMMUNOGENIC MPB70. |
| 1314 | DM01753B | 6.47 | 1.750e−13 | 538-547 | DM01753 | 6 kw OSTEOBLAST MAJOR |
| | | | | | | IMMUNOGENIC MPB70. |
| 1314 | DM01753B | 6.47 | 8.767e−09 | 139-148 | DM01753 | 6 kw OSTEOBLAST MAJOR |
| | | | | | | IMMUNOGENIC MPB70. |
| 1314 | DM01753C | 17.64 | 1.735e−09 | 199-237 | DM01753 | 6 kw OSTEOBLAST MAJOR |
| | | | | | | IMMUNOGENIC MPB70. |
| 1315 | BL00046 | 12.95 | 1.000e−40 | 36-90 | BL00046 | Histone H2A proteins. |
| 1315 | PD01672F | 8.62 | 7.029e−09 | 35-68 | PD01672 | + TRANSPORT EXCHANGER |
| | | | | | | NA H TRANS. |
| 1315 | PR00620A | 11.03 | 1.429e−24 | 16-38 | PR00620 | HISTONE H2A SIGNATURE |
| 1315 | PR00620B | 8.47 | 8.909e−17 | 46-61 | PR00620 | HISTONE H2A SIGNATURE |
| 1315 | PR00620C | 10.19 | 2.862e−15 | 61-74 | PR00620 | HISTONE H2A SIGNATURE |
| 1315 | PR00620D | 11.39 | 3.172e−15 | 75-89 | PR00620 | HISTONE H2A SIGNATURE |
| 1315 | PR00620E | 8.91 | 3.025e−12 | 89-101 | PR00620 | HISTONE H2A SIGNATURE |
| 1315 | PR00620F | 7.29 | 6.226e−16 | 102-120 | PR00620 | HISTONE H2A SIGNATURE |
| 1317 | BL00188 | 30.29 | 9.036e−32 | 640-685 | BL00188 | Biotin-requiring |
| | | | | | | enzymes attachment site |
| | | | | | | proteins. |
| 1317 | BL00189A | 19.58 | 5.279e−12 | 651-685 | BL00189 | 2-oxo acid |
| | | | | | | dehydrogenases |
| | | | | | | acyltransferase |
| | | | | | | component lipoyl bi. |
| 1317 | BL00866B | 36.29 | 2.286e−31 | 234-279 | BL00866 | Carbamoyl-phosphate |
| | | | | | | synthase subdomain |
| | | | | | | proteins. |
| 1317 | BL00866C | 23.26 | 1.621e−18 | 320-354 | BL00866 | Carbamoyl-phosphate |
| | | | | | | synthase subdomain |
| | | | | | | proteins. |
| 1317 | BL00866C | 23.26 | 5.034e−18 | 9-43 | BL00866 | Carbamoyl-phosphate |
| | | | | | | synthase subdomain |
| | | | | | | proteins. |
| 1317 | PD00187 | 10.12 | 3.118e−15 | 188-203 | PD00187 | LIGASE BIOSYNTHESIS |
| | | | | | | SYNTHETASE. |
| 1318 | BL00226B | 23.86 | 6.128e−09 | 77-124 | BL00226 | Intermediate filaments |
| | | | | | | proteins. |
| 1318 | BL00326D | 8.76 | 1.000e−40 | 62-102 | BL00326 | Tropomyosins proteins. |
| 1318 | BL00326D | 8.76 | 7.729e−09 | 6-46 | BL00326 | Tropomyosins proteins. |
| 1318 | PR00194D | 9.57 | 1.300e−23 | 17-40 | PR00194 | TROPOMYOSIN SIGNATURE |
| 1318 | PR00194E | 8.74 | 3.250e−30 | 73-98 | PR00194 | TROPOMYOSIN SIGNATURE |
| 1323 | PD01485A | 12.50 | 7.250e−10 | 129-141 | PD01485 | INITIATION FACTOR |
| | | | | | | PROTEIN TRANSLATION |
| | | | | | | BIO. |
| 1323 | PD01485B | 23.41 | 1.333e−37 | 211-251 | PD01485 | INITIATION FACTOR |
| | | | | | | PROTEIN TRANSLATION |
| | | | | | | BIO. |
| 1323 | PD01485C | 20.40 | 4.600e−36 | 257-291 | PD01485 | INITIATION FACTOR |
| | | | | | | PROTEIN TRANSLATION |
| | | | | | | BIO. |
| 1325 | PR00308A | 5.90 | 6.918e−11 | 45-59 | PR00308 | TYPE I ANTIFREEZE |
| | | | | | | PROTEIN SIGNATURE |
| 1325 | PR00308C | 3.83 | 7.312e−09 | 48-57 | PR00308 | TYPE I ANTIFREEZE |
| | | | | | | PROTEIN SIGNATURE |
| 1325 | PR00456E | 3.06 | 9.316e−09 | 41-55 | PR00456 | RIBOSOMAL PROTEIN P2 |
| | | | | | | SIGNATURE |
| 1326 | BL00464A | 29.41 | 9.700e−23 | 17-53 | BL00464 | Ribosomal protein L22 |
| | | | | | | proteins. |
| 1326 | BL00464B | 28.48 | 4.960e−35 | 106-150 | BL00464 | Ribosomal protein L22 |
| | | | | | | proteins. |
| 1328 | BL00854A | 33.93 | 4.405e−24 | 48-93 | BL00854 | Proteasome B-type |
| | | | | | | subunits proteins. |
| 1328 | BL00854C | 29.92 | 5.615e−12 | 154-182 | BL00854 | Proteasome B-type |
| | | | | | | subunits proteins. |
| 1328 | PR00141A | 11.36 | 8.800e−11 | 51-66 | PR00141 | PROTEASOME COMPONENT |
| | | | | | | SIGNATURE |
| 1329 | BL00509A | 14.33 | 3.400e−12 | 520-530 | BL00509 | Ras GTPase-activating |
| | | | | | | proteins. |
| 1329 | BL00509B | 10.28 | 4.960e−11 | 771-781 | BL00509 | Ras GTPase-activating |
| | | | | | | proteins. |
| 1329 | PF00168C | 27.49 | 7.375e−09 | 653-678 | PF00168 | C2 domain proteins. |
| 1329 | PR00401A | 14.00 | 7.075e−09 | 20-34 | PR00401 | SH2 DOMAIN SIGNATURE |
| 1329 | PR00401E | 14.10 | 5.235e−09 | 84-98 | PR00401 | SH2 DOMAIN SIGNATURE |
| 1330 | BL00113A | 12.74 | 8.338e−09 | 223-239 | BL00113 | Adenylate kinase |
| | | | | | | proteins. |
| 1330 | BL00674A | 16.91 | 2.636e−17 | 184-204 | BL00674 | AAA-protein family |
| | | | | | | proteins. |
| 1330 | BL00674B | 4.46 | 6.294e−25 | 219-240 | BL00674 | AAA-protein family |
| | | | | | | proteins. |
| 1330 | BL00674C | 22.60 | 3.077e−35 | 252-294 | BL00674 | AAA-protein family |
| | | | | | | proteins. |
| 1330 | BL00674D | 23.41 | 1.000e−40 | 311-357 | BL00674 | AAA-protein family |
| | | | | | | proteins. |
| 1330 | BL00674E | 15.24 | 9.143e−17 | 389-408 | BL00674 | AAA-protein family |
| | | | | | | proteins. |
| 1330 | BL00675A | 24.86 | 4.488e−09 | 222-265 | BL00675 | Sigma-54 interaction |
| | | | | | | domain proteins ATP- |
| | | | | | | binding region A |
| | | | | | | proteins. |
| 1330 | PR00300A | 9.56 | 3.318e−11 | 222-240 | PR00300 | ATP-DEPENDENT CLP |
| | | | | | | PROTEASE ATP-BINDING |
| | | | | | | SUBUNIT SIGNATURE |
| 1330 | PR00819B | 10.83 | 9.100e−10 | 221-236 | PR00819 | CBXX/CFQX SUPERFAMILY |
| | | | | | | SIGNATURE |
| 1330 | PR00830A | 8.41 | 9.018e−13 | 226-245 | PR00830 | ENDOPEPTIDASE LA (LON) |
| | | | | | | SERINE PROTEASE (S16) |
| | | | | | | SIGNATURE |
| 1330 | PR00918A | 13.76 | 7.181e−10 | 216-236 | PR00918 | CALICIVIRUS NON- |
| | | | | | | STRUCTURAL POLYPROTEIN |
| | | | | | | FAMILY SIGNATURE |
| 1331 | BL00284A | 15.64 | 8.714e−16 | 77-100 | BL00284 | Serpins proteins. |
| 1331 | BL00284B | 17.99 | 8.091e−12 | 176-196 | BL00284 | Serpins proteins. |
| 1331 | BL00284C | 28.56 | 3.700e−26 | 203-244 | BL00284 | Serpins proteins. |
| 1331 | BL00284D | 16.34 | 7.279e−12 | 312-338 | BL00284 | Serpins proteins. |
| 1331 | BL00284E | 19.15 | 1.333e−17 | 391-415 | BL00284 | Serpins proteins. |
| 1332 | BL00250A | 21.24 | 6.000e−15 | 211-246 | BL00250 | TGF-beta family |
| | | | | | | proteins. |
| 1332 | BL00250B | 27.37 | 1.305e−09 | 272-307 | BL00250 | TGF-beta family |
| | | | | | | proteins. |
| 1339 | BL00831A | 16.12 | 5.598e−09 | 32-60 | BL00831 | Ribosomal protein L27 |
| | | | | | | proteins. |
| 1339 | BL00831B | 21.15 | 6.348e−19 | 61-100 | BL00831 | Ribosomal protein L27 |
| | | | | | | proteins. |
| 1339 | PR00063A | 11.71 | 1.614e−09 | 34-58 | PR00063 | RIBOSOMAL PROTEIN L27 |
| | | | | | | SIGNATURE |
| 1339 | PR00063B | 15.24 | 9.700e−11 | 59-83 | PR00063 | RIBOSOMAL PROTEIN L27 |
| | | | | | | SIGNATURE |
| 1342 | BL00652A | 3.16 | 4.000e−09 | 22-28 | BL00652 | TNFR/NGFR family |
| | | | | | | cysteine-rich region |
| | | | | | | proteins. |
| 1342 | PF00084B | 9.45 | 4.375e−10 | 122-133 | PF00084 | Sushi domain proteins |
| | | | | | | (SCR repeat proteins. |
| 1342 | PF00084C | 11.25 | 3.571e−10 | 274-283 | PF00084 | Sushi domain proteins |
| | | | | | | (SCR repeat proteins. |
| 1349 | BL00113A | 12.74 | 9.031e−09 | 187-203 | BL00113 | Adenylate kinase |
| | | | | | | proteins. |
| 1349 | BL00516B | 29.10 | 1.000e−08 | 314-356 | BL00516 | Alkylbase DNA |
| | | | | | | glycosidases alkA |
| | | | | | | family proteins. |
| 1349 | BL00674A | 16.91 | 7.000e−15 | 148-168 | BL00674 | AAA-protein family |
| | | | | | | proteins. |
| 1349 | BL00674B | 4.46 | 4.000e−27 | 183-204 | BL00674 | AAA-protein family |
| | | | | | | proteins. |
| 1349 | BL00674C | 22.60 | 4.273e−38 | 216-258 | BL00674 | AAA-protein family |
| | | | | | | proteins. |
| 1349 | BL00674D | 23.41 | 6.500e−39 | 275-321 | BL00674 | AAA-protein family |
| | | | | | | proteins. |
| 1349 | BL00674E | 15.24 | 6.478e−16 | 353-372 | BL00674 | AAA-protein family |
| | | | | | | proteins. |
| 1349 | BL00675A | 24.86 | 8.538e−09 | 186-229 | BL00675 | Sigma-54 interaction |
| | | | | | | domain proteins ATP- |
| | | | | | | binding region A |
| | | | | | | proteins. |
| 1349 | PR00300A | 9.56 | 4.409e−11 | 186-204 | PR00300 | ATP-DEPENDENT CLP |
| | | | | | | PROTEASE ATP-BINDING |
| | | | | | | SUBUNIT SIGNATURE |
| 1349 | PR00819B | 10.83 | 1.318e−09 | 185-200 | PR00819 | CBXX/CFQX SUPERFAMILY |
| | | | | | | SIGNATURE |
| 1349 | PR00830A | 8.41 | 5.810e−12 | 190-209 | PR00830 | ENDOPEPTIDASE LA (LON) |
| | | | | | | SERINE PROTEASE (S16) |
| | | | | | | SIGNATURE |
| 1349 | PR00918A | 13.76 | 5.988e−10 | 180-200 | PR00918 | CALICIVIRUS NON- |
| | | | | | | STRUCTURAL POLYPROTEIN |
| | | | | | | FAMILY SIGNATURE |
| 1352 | BL00904A | 8.30 | 9.936e−09 | 320-369 | BL00904 | Protein |
| | | | | | | prenyltransferases |
| | | | | | | alpha subunit repeat |
| | | | | | | proteins proteins. |
| 1352 | DM00215 | 19.43 | 6.464e−10 | 304-336 | DM00215 | PROLINE-RICH PROTEIN 3. |
| 1353 | BL00674A | 16.91 | 5.500e−13 | 174-194 | BL00674 | AAA-protein family |
| | | | | | | proteins. |
| 1353 | BL00674A | 16.91 | 5.696e−09 | 1-21 | BL00674 | AAA-protein family |
| | | | | | | proteins. |
| 1353 | BL00674B | 4.46 | 6.500e−24 | 209-230 | BL00674 | AAA-protein family |
| | | | | | | proteins. |
| 1353 | BL00674C | 22.60 | 5.846e−35 | 242-284 | BL00674 | AAA-protein family |
| | | | | | | proteins. |
| 1353 | BL00674D | 23.41 | 1.000e−40 | 301-347 | BL00674 | AAA-protein family |
| | | | | | | proteins. |
| 1353 | BL00674E | 15.24 | 8.826e−16 | 379-398 | BL00674 | AAA-protein family |
| | | | | | | proteins. |
| 1353 | BL00675A | 24.86 | 5.613e−09 | 212-255 | BL00675 | Sigma-54 interaction |
| | | | | | | domain proteins ATP- |
| | | | | | | binding region A |
| | | | | | | proteins. |
| 1353 | PR00300A | 9.56 | 1.887e−10 | 212-230 | PR00300 | ATP-DEPENDENT CLP |
| | | | | | | PROTEASE ATP-BINDING |
| | | | | | | SUBUNIT SIGNATURE |
| 1353 | PR00819B | 10.83 | 1.529e−09 | 211-226 | PR00819 | CBXX/CFQX SUPERFAMILY |
| | | | | | | SIGNATURE |
| 1353 | PR00830A | 8.41 | 2.714e−11 | 216-235 | PR00830 | ENDOPEPTIDASE LA (LON) |
| | | | | | | SERINE PROTEASE (S16) |
| | | | | | | SIGNATURE |
| 1353 | PR00887F | 12.74 | 7.889e−09 | 267-284 | PR00887 | STRUCTURE-SPECIFIC |
| | | | | | | RECOGNITION PROTEIN |
| | | | | | | SIGNATURE |
| 1354 | BL00678 | 9.67 | 1.000e−08 | 233-243 | BL00678 | Trp-Asp (WD) repeat |
| | | | | | | proteins proteins. |
| 1354 | BL00678 | 9.67 | 7.158e−09 | 184-194 | BL00678 | Trp-Asp (WD) repeat |
| | | | | | | proteins proteins. |
| 1354 | PR00320A | 16.74 | 2.853e−10 | 74-88 | PR00320 | G-PROTEIN BETA WD-40 |
| | | | | | | REPEAT SIGNATURE |
| 1354 | PR00320A | 16.74 | 4.706e−10 | 28-42 | PR00320 | G-PROTEIN BETA WD-40 |
| | | | | | | REPEAT SIGNATURE |
| 1354 | PR00320A | 16.74 | 6.049e−09 | 231-245 | PR00320 | G-PROTEIN BETA WD-40 |
| | | | | | | REPEAT SIGNATURE |
| 1354 | PR00320B | 12.19 | 8.875e−09 | 182-196 | PR00320 | G-PROTEIN BETA WD-40 |
| | | | | | | REPEAT SIGNATURE |
| 1354 | PR00320C | 13.01 | 5.320e−10 | 74-88 | PR00320 | G-PROTEIN BETA WD-40 |
| | | | | | | REPEAT SIGNATURE |
| 1354 | PR00320C | 13.01 | 5.680e−10 | 28-42 | PR00320 | G-PROTEIN BETA WD-40 |
| | | | | | | REPEAT SIGNATURE |
| 1354 | PR00320C | 13.01 | 9.500e−12 | 182-196 | PR00320 | G-PROTEIN BETA WD-40 |
| | | | | | | REPEAT SIGNATURE |
| 1357 | BL00115Z | 3.12 | 8.412e−09 | 172-220 | BL00115 | Eukaryotic RNA |
| | | | | | | polymerase II |
| | | | | | | heptapeptide repeat |
| | | | | | | proteins. |
| 1357 | BL00412C | 10.28 | 6.932e−09 | 183-216 | BL00412 | Neuromodulin (GAP-43) |
| | | | | | | proteins. |
| 1362 | BL01145A | 13.73 | 1.000e−40 | 3-44 | BL01145 | Ribosomal protein L34e |
| | | | | | | proteins. |
| 1362 | BL01145B | 14.65 | 2.636e−20 | 88-110 | BL01145 | Ribosomal protein L34e |
| | | | | | | proteins. |
| 1363 | PR00058A | 16.66 | 1.375e−26 | 20-39 | PR00058 | RIBOSOMAL PROTEIN L5 |
| | | | | | | SIGNATURE |
| 1363 | PR00058B | 8.75 | 1.529e−27 | 43-62 | PR00058 | RIBOSOMAL PROTEIN L5 |
| | | | | | | SIGNATURE |
| 1365 | BL00112A | 22.23 | 1.000e−40 | 94-135 | BL00112 | ATP: guanido |
| | | | | | | phosphotransferases |
| | | | | | | proteins. |
| 1365 | BL00112B | 15.87 | 9.217e−31 | 155-186 | BL00112 | ATP: guanido |
| | | | | | | phosphotransferases |
| | | | | | | proteins. |
| 1365 | BL00112C | 21.77 | 1.000e−40 | 187-240 | BL00112 | ATP: guanido |
| | | | | | | phosphotransferases |
| | | | | | | proteins. |
| 1365 | BL00112D | 12.22 | 1.000e−40 | 242-286 | BL00112 | ATP: guanido |
| | | | | | | phosphotransferases |
| | | | | | | proteins. |
| 1365 | BL00112E | 17.75 | 3.455e−37 | 304-333 | BL00112 | ATP: guanido |
| | | | | | | phosphotransferases |
| | | | | | | proteins. |
| 1365 | BL00112F | 21.37 | 1.000e−40 | 350-400 | BL00112 | ATP: guanido |
| | | | | | | phosphotransferases |
| | | | | | | proteins. |
| 1366 | PR00800A | 15.23 | 9.565e−09 | 99-118 | PR00800 | AROMATIC-L-AMINO-ACID |
| | | | | | | DECARBOXYLASE SIGNATURE |
| 1367 | BL00213A | 12.95 | 9.526e−10 | 37-50 | BL00213 | Lipocalin proteins. |
| 1367 | BL00213B | 8.78 | 8.000e−10 | 120-130 | BL00213 | Lipocalin proteins. |
| 1367 | PR00179A | 13.78 | 5.680e−10 | 37-49 | PR00179 | LIPOCALIN SIGNATURE |
| 1367 | PR00179B | 9.56 | 1.000e−12 | 120-132 | PR00179 | LIPOCALIN SIGNATURE |
| 1367 | PR00179C | 19.02 | 1.000e−10 | 148-163 | PR00179 | LIPOCALIN SIGNATURE |
| 1369 | PR00050A | 11.28 | 5.865e−09 | 65-80 | PR00050 | COLD SHOCK PROTEIN |
| | | | | | | SIGNATURE |
| 1370 | PR00456F | 5.86 | 6.400e−12 | 101-112 | PR00456 | RIBOSOMAL PROTEIN P2 |
| | | | | | | SIGNATURE |
| 1371 | BL00194 | 12.16 | 9.438e−15 | 24-36 | BL00194 | Thioredoxin family |
| | | | | | | proteins. |
| 1371 | PR00421A | 10.15 | 2.286e−10 | 23-31 | PR00421 | THIOREDOXIN FAMILY |
| | | | | | | SIGNATURE |
| 1371 | PR00421B | 11.40 | 6.625e−13 | 31-40 | PR00421 | THIOREDOXIN FAMILY |
| | | | | | | SIGNATURE |
| 1371 | PR00421C | 13.60 | 8.615e−12 | 70-81 | PR00421 | THIOREDOXIN FAMILY |
| | | | | | | SIGNATURE |
| 1372 | BL00478B | 14.79 | 3.348e−14 | 157-171 | BL00478 | LIM domain proteins. |
| 1372 | BL00478B | 14.79 | 4.913e−14 | 48-62 | BL00478 | LIM domain proteins. |
| 1374 | BL00030B | 7.03 | 8.105e−09 | 50-59 | BL00030 | Eukaryotic RNA-binding |
| | | | | | | region RNP-1 proteins. |
| 1377 | BL00154A | 11.86 | 5.500e−16 | 188-205 | BL00154 | E1-E2 ATPases |
| | | | | | | phosphorylation site |
| | | | | | | proteins. |
| 1377 | BL00154B | 15.44 | 2.800e−31 | 332-368 | BL00154 | E1-E2 ATPases |
| | | | | | | phosphorylation site |
| | | | | | | proteins. |
| 1377 | BL00154C | 12.38 | 6.000e−23 | 370-388 | BL00154 | E1-E2 ATPases |
| | | | | | | phosphorylation site |
| | | | | | | proteins. |
| 1377 | BL00154D | 12.57 | 3.769e−13 | 508-518 | BL00154 | E1-E2 ATPases |
| | | | | | | phosphorylation site |
| | | | | | | proteins. |
| 1377 | BL00154E | 20.37 | 8.615e−38 | 593-633 | BL00154 | E1-E2 ATPases |
| | | | | | | phosphorylation site |
| | | | | | | proteins. |
| 1377 | BL00154F | 8.23 | 3.813e−27 | 712-735 | BL00154 | E1-E2 ATPases |
| | | | | | | phosphorylation site |
| | | | | | | proteins. |
| 1377 | BL00154G | 21.18 | 9.526e−30 | 738-771 | BL00154 | E1-E2 ATPases |
| | | | | | | phosphorylation site |
| | | | | | | proteins. |
| 1377 | BL01047B | 19.73 | 8.000e−09 | 721-741 | BL01047 | Heavy-metal-associated |
| | | | | | | domain proteins. |
| 1377 | BL01228D | 17.44 | 3.684e−12 | 713-737 | BL01228 | Hypothetical cof family |
| | | | | | | proteins. |
| 1377 | PR00119A | 17.34 | 3.000e−15 | 214-228 | PR00119 | P-TYPE CATION- |
| | | | | | | TRANSPORTING ATPASE |
| | | | | | | SUPERFAMILY SIGNATURE |
| 1377 | PR00119B | 13.94 | 2.800e−20 | 374-388 | PR00119 | P-TYPE CATION- |
| | | | | | | TRANSPORTING ATPASE |
| | | | | | | SUPERFAMILY SIGNATURE |
| 1377 | PR00119C | 11.01 | 4.500e−12 | 587-598 | PR00119 | P-TYPE CATION- |
| | | | | | | TRANSPORTING ATPASE |
| | | | | | | SUPERFAMILY SIGNATURE |
| 1377 | PR00119D | 9.56 | 3.571e−13 | 609-619 | PR00119 | P-TYPE CATION- |
| | | | | | | TRANSPORTING ATPASE |
| | | | | | | SUPERFAMILY SIGNATURE |
| 1377 | PR00119E | 8.48 | 5.200e−24 | 715-734 | PR00119 | P-TYPE CATION- |
| | | | | | | TRANSPORTING ATPASE |
| | | | | | | SUPERFAMILY SIGNATURE |
| 1377 | PR00119F | 11.81 | 7.750e−13 | 739-751 | PR00119 | P-TYPE CATION- |
| | | | | | | TRANSPORTING ATPASE |
| | | | | | | SUPERFAMILY SIGNATURE |
| 1377 | PR00120C | 9.90 | 5.800e−19 | 715-731 | PR00120 | H+-TRANSPORTING ATPASE |
| | | | | | | (PROTON PUMP) SIGNATURE |
| 1377 | PR00121A | 6.71 | 9.053e−18 | 103-117 | PR00121 | SODIUM/POTASSIUM- |
| | | | | | | TRANSPORTING ATPASE |
| | | | | | | SIGNATURE |
| 1377 | PR00121B | 7.83 | 4.214e−26 | 130-150 | PR00121 | SODIUM/POTASSIUM- |
| | | | | | | TRANSPORTING ATPASE |
| | | | | | | SIGNATURE |
| 1377 | PR00121C | 9.40 | 1.000e−24 | 294-316 | PR00121 | SODIUM/POTASSIUM- |
| | | | | | | TRANSPORTING ATPASE |
| | | | | | | SIGNATURE |
| 1377 | PR00121D | 16.72 | 1.209e−28 | 367-388 | PR00121 | SODIUM/POTASSIUM- |
| | | | | | | TRANSPORTING ATPASE |
| | | | | | | SIGNATURE |
| 1377 | PR00121E | 13.97 | 4.667e−23 | 505-523 | PR00121 | SODIUM/POTASSIUM- |
| | | | | | | TRANSPORTING ATPASE |
| | | | | | | SIGNATURE |
| 1377 | PR00121F | 6.70 | 7.750e−27 | 787-808 | PR00121 | SODIUM/POTASSIUM- |
| | | | | | | TRANSPORTING ATPASE |
| | | | | | | SIGNATURE |
| 1377 | PR00121G | 6.89 | 8.875e−26 | 854-874 | PR00121 | SODIUM/POTASSIUM- |
| | | | | | | TRANSPORTING ATPASE |
| | | | | | | SIGNATURE |
| 1377 | PR00121H | 12.14 | 5.950e−26 | 916-936 | PR00121 | SODIUM/POTASSIUM- |
| | | | | | | TRANSPORTING ATPASE |
| | | | | | | SIGNATURE |
| 1377 | PR00121I | 15.47 | 8.500e−28 | 950-974 | PR00121 | SODIUM/POTASSIUM- |
| | | | | | | TRANSPORTING ATPASE |
| | | | | | | SIGNATURE |
| 1379 | PR00833H | 2.30 | 6.423e−09 | 329-343 | PR00833 | POLLEN ALLERGEN POA PI |
| | | | | | | SIGNATURE |
| 1381 | BL00478B | 14.79 | 2.800e−11 | 279-293 | BL00478 | LIM domain proteins. |
| 1382 | BL01294A | 5.15 | 1.571e−09 | 23-34 | BL01294 | Uncharacterized protein |
| | | | | | | family UPF0038 |
| | | | | | | proteins. |
| 1382 | PR00988A | 6.39 | 8.500e−17 | 20-37 | PR00988 | URIDINE KINASE |
| | | | | | | SIGNATURE |
| 1382 | PR00988B | 11.60 | 2.915e−09 | 57-68 | PR00988 | URIDINE KINASE |
| | | | | | | SIGNATURE |
| 1382 | PR00988C | 13.64 | 6.108e−14 | 104-119 | PR00988 | URIDINE KINASE |
| | | | | | | SIGNATURE |
| 1382 | PR00988D | 5.95 | 6.878e−10 | 160-170 | PR00988 | URIDINE KINASE |
| | | | | | | SIGNATURE |
| 1382 | PR00988E | 8.27 | 3.872e−11 | 174-185 | PR00988 | URIDINE KINASE |
| | | | | | | SIGNATURE |
| 1382 | PR00988F | 12.23 | 7.828e−15 | 196-209 | PR00988 | URIDINE KINASE |
| | | | | | | SIGNATURE |
| 1383 | BL01115A | 10.22 | 2.768e−10 | 60-103 | BL01115 | GTP-binding nuclear |
| | | | | | | protein ran proteins. |
| 1383 | BL01215A | 9.75 | 3.968e−09 | 57-83 | BL01215 | Mrp family proteins. |
| 1383 | PR00449A | 13.20 | 4.706e−19 | 60-81 | PR00449 | TRANSFORMING PROTEIN |
| | | | | | | P21 RAS SIGNATURE |
| 1383 | PR00449B | 14.34 | 7.000e−15 | 83-99 | PR00449 | TRANSFORMING PROTEIN |
| | | | | | | P21 RAS SIGNATURE |
| 1383 | PR00449C | 17.27 | 8.579e−18 | 100-122 | PR00449 | TRANSFORMING PROTEIN |
| | | | | | | P21 RAS SIGNATURE |
| 1383 | PR00449D | 10.79 | 3.769e−16 | 163-176 | PR00449 | TRANSFORMING PROTEIN |
| | | | | | | P21 RAS SIGNATURE |
| 1383 | PR00449E | 13.50 | 1.450e−16 | 199-221 | PR00449 | TRANSFORMING PROTEIN |
| | | | | | | P21 RAS SIGNATURE |
| 1384 | PR00495A | 9.18 | 3.700e−25 | 27-45 | PR00495 | OBESITY FACTOR (LEPTIN) |
| | | | | | | SIGNATURE |
| 1384 | PR00495B | 10.99 | 1.099e−24 | 47-65 | PR00495 | OBESITY FACTOR (LEPTIN) |
| | | | | | | SIGNATURE |
| 1384 | PR00495C | 10.22 | 7.492e−26 | 74-94 | PR00495 | OBESITY FACTOR (LEPTIN) |
| | | | | | | SIGNATURE |
| 1384 | PR00495D | 11.96 | 3.571e−24 | 97-115 | PR00495 | OBESITY FACTOR (LEPTIN) |
| | | | | | | SIGNATURE |
| 1384 | PR00495E | 13.09 | 5.114e−26 | 118-138 | PR00495 | OBESITY FACTOR (LEPTIN) |
| | | | | | | SIGNATURE |
| 1384 | PR00495F | 7.55 | 3.492e−24 | 147-167 | PR00495 | OBESITY FACTOR (LEPTIN) |
| | | | | | | SIGNATURE |
| 1384 | PR00495G | 6.74 | 1.529e−21 | 168-183 | PR00495 | OBESITY FACTOR (LEPTIN) |
| | | | | | | SIGNATURE |
| 1386 | BL00039A | 18.44 | 4.600e−30 | 57-95 | BL00039 | DEAD-box subfamily ATP- |
| | | | | | | dependent helicases |
| | | | | | | proteins. |
| 1386 | BL00039B | 19.19 | 2.200e−20 | 97-122 | BL00039 | DEAD-box subfamily ATP- |
| | | | | | | dependent helicases |
| | | | | | | proteins. |
| 1386 | BL00039C | 15.63 | 2.500e−21 | 181-204 | BL00039 | DEAD-box subfamily ATP- |
| | | | | | | dependent helicases |
| | | | | | | proteins. |
| 1386 | BL00039D | 21.67 | 1.000e−40 | 330-375 | BL00039 | DEAD-box subfamily ATP- |
| | | | | | | dependent helicases |
| | | | | | | proteins. |
| 1386 | DM01537B | 21.63 | 1.786e−11 | 304-350 | DM01537 | kw SKI2W SKI2 NUCLEOLAR |
| | | | | | | HELICASE. |
| 1386 | PF00271 | 7.99 | 1.000e−08 | 361-368 | PF00271 | Helicases conserved C- |
| | | | | | | terminal domain |
| | | | | | | proteins. |
| 1388 | PD00078B | 13.14 | 7.600e−12 | 141-153 | PD00078 | REPEAT PROTEIN ANK |
| | | | | | | NUCLEAR ANKYR. |
| 1388 | PF00023A | 16.03 | 1.750e−10 | 187-202 | PF00023 | Ank repeat proteins. |
| 1388 | PF00023A | 16.03 | 3.250e−09 | 148-163 | PF00023 | Ank repeat proteins. |
| 1388 | PF00023B | 14.20 | 9.100e−12 | 144-153 | PF00023 | Ank repeat proteins. |
| 1390 | BL00492D | 13.23 | 5.865e−09 | 315-351 | BL00492 | Clusterin proteins. |
| 1390 | PR00497A | 6.92 | 5.553e−10 | 554-571 | PR00497 | NEUTROPHIL CYTOSOL |
| | | | | | | FACTOR P40 SIGNATURE |
| 1391 | BL01118A | 12.46 | 4.000e−13 | 36-50 | BL01118 | Translation initiation |
| | | | | | | factor SUI1 proteins. |
| 1391 | BL01118B | 26.75 | 9.308e−33 | 53-90 | BL01118 | Translation initiation |
| | | | | | | factor SUI1 proteins. |
| 1395 | PF00429 | 31.08 | 7.300e−15 | 383-432 | PF00429 | ENV polyprotein (coat |
| | | | | | | polyprotein) |
| 1395 | PR00415D | 12.72 | 8.403e−09 | 328-343 | PR00415 | ACONITASE FAMILY |
| | | | | | | SIGNATURE |
| 1395 | PR00783F | 12.33 | 3.700e−09 | 446-466 | PR00783 | MAJOR INTRINSIC PROTEIN |
| | | | | | | FAMILY SIGNATURE |
| 1396 | DM01022C | 19.84 | 3.571e−09 | 103-122 | DM01022 | LRR REPEAT. |
| 1396 | PR00019A | 11.19 | 2.565e−10 | 131-144 | PR00019 | LEUCINE-RICH REPEAT |
| | | | | | | SIGNATURE |
| 1396 | PR00019B | 11.36 | 4.000e−11 | 268-281 | PR00019 | LEUCINE-RICH REPEAT |
| | | | | | | SIGNATURE |
| 1396 | PR00019B | 11.36 | 4.600e−10 | 220-233 | PR00019 | LEUCINE-RICH REPEAT |
| | | | | | | SIGNATURE |
| 1396 | PR00019B | 11.36 | 7.840e−09 | 128-141 | PR00019 | LEUCINE-RICH REPEAT |
| | | | | | | SIGNATURE |
| 1397 | BL00299 | 28.84 | 1.000e−40 | 16-67 | BL00299 | Ubiquitin domain |
| | | | | | | proteins. |
| 1397 | PD01457A | 16.51 | 1.000e−40 | 77-121 | PD01457 | RIBOSOMAL PROTEIN 40S |
| | | | | | | ZINC-FINGER METAL. |
| 1397 | PD01457B | 13.00 | 1.000e−31 | 124-148 | PD01457 | RIBOSOMAL PROTEIN 40S |
| | | | | | | ZINC-FINGER METAL. |
| 1397 | PR00348A | 7.86 | 2.000e−26 | 11-31 | PR00348 | UBIQUITIN SIGNATURE |
| 1397 | PR00348B | 5.78 | 2.800e−27 | 32-52 | PR00348 | UBIQUITIN SIGNATURE |
| 1397 | PR00348C | 20.03 | 1.643e−27 | 53-74 | PR00348 | UBIQUITIN SIGNATURE |
| 1398 | BL00280 | 24.61 | 3.000e−29 | 165-208 | BL00280 | Pancreatic trypsin |
| | | | | | | inhibitor (Kunitz) |
| | | | | | | family proteins. |
| 1398 | BL00280 | 24.61 | 4.857e−34 | 43-86 | BL00280 | Pancreatic trypsin |
| | | | | | | inhibitor (Kunitz) |
| | | | | | | family proteins. |
| 1398 | BL00280 | 24.61 | 5.215e−10 | 104-147 | BL00280 | Pancreatic trypsin |
| | | | | | | inhibitor (Kunitz) |
| | | | | | | family proteins. |
| 1398 | PR00759A | 14.51 | 3.045e−11 | 33-47 | PR00759 | BASIC PROTEASE (KUNITZ- |
| | | | | | | TYPE) INHIBITOR FAMILY |
| | | | | | | SIGNATURE |
| 1398 | PR00759A | 14.51 | 7.900e−09 | 155-169 | PR00759 | BASIC PROTEASE (KUNITZ- |
| | | | | | | TYPE) INHIBITOR FAMILY |
| | | | | | | SIGNATURE |
| 1398 | PR00759B | 11.26 | 1.231e−09 | 183-193 | PR00759 | BASIC PROTEASE (KUNITZ- |
| | | | | | | TYPE) INHIBITOR FAMILY |
| | | | | | | SIGNATURE |
| 1398 | PR00759B | 11.26 | 1.290e−10 | 61-71 | PR00759 | BASIC PROTEASE (KUNITZ- |
| | | | | | | TYPE) INHIBITOR FAMILY |
| | | | | | | SIGNATURE |
| 1398 | PR00759C | 14.15 | 3.483e−13 | 193-208 | PR00759 | BASIC PROTEASE (KUNITZ- |
| | | | | | | TYPE) INHIBITOR FAMILY |
| | | | | | | SIGNATURE |
| 1398 | PR00759C | 14.15 | 5.636e−12 | 71-86 | PR00759 | BASIC PROTEASE (KUNITZ- |
| | | | | | | TYPE) INHIBITOR FAMILY |
| | | | | | | SIGNATURE |
| 1399 | BL00388A | 23.14 | 5.875e−40 | 5-50 | BL00388 | Proteasome A-type |
| | | | | | | subunits proteins. |
| 1399 | BL00388B | 31.38 | 6.538e−29 | 64-105 | BL00388 | Proteasome A-type |
| | | | | | | subunits proteins. |
| 1399 | BL00388C | 18.79 | 2.000e−22 | 119-140 | BL00388 | Proteasome A-type |
| | | | | | | subunits proteins. |
| 1399 | BL00388D | 20.71 | 1.391e−26 | 147-177 | BL00388 | Proteasome A-type |
| | | | | | | subunits proteins. |
| 1399 | BL00854A | 33.93 | 9.000e−11 | 36-81 | BL00854 | Proteasome B-type |
| | | | | | | subunits proteins. |
| 1399 | PF00227 | 14.68 | 5.950e−09 | 12-23 | PF00227 | Proteasome A-type and |
| | | | | | | B-type. |
| 1401 | BL00113A | 12.74 | 9.031e−09 | 171-187 | BL00113 | Adenylate kinase |
| | | | | | | proteins. |
| 1401 | BL00674A | 16.91 | 4.857e−14 | 132-152 | BL00674 | AAA-protein family |
| | | | | | | proteins. |
| 1401 | BL00674B | 4.46 | 5.235e−25 | 167-188 | BL00674 | AAA-protein family |
| | | | | | | proteins. |
| 1401 | BL00674C | 22.60 | 9.308e−35 | 200-242 | BL00674 | AAA-protein family |
| | | | | | | proteins. |
| 1401 | BL00674D | 23.41 | 4.176e−40 | 259-305 | BL00674 | AAA-protein family |
| | | | | | | proteins. |
| 1401 | BL00674E | 15.24 | 8.200e−20 | 337-356 | BL00674 | AAA-protein family |
| | | | | | | proteins. |
| 1401 | PR00051A | 10.68 | 8.382e−09 | 167-187 | PR00051 | BACTERIAL CHROMOSOMAL |
| | | | | | | REPLICATION INITIATOR |
| | | | | | | (DNAA) SIGNATURE |
| 1401 | PR00300A | 9.56 | 4.682e−11 | 170-188 | PR00300 | ATP-DEPENDENT CLP |
| | | | | | | PROTEASE ATP-BINDING |
| | | | | | | SUBUNIT SIGNATURE |
| 1401 | PR00819B | 10.83 | 2.482e−09 | 169-184 | PR00819 | CBXX/CFQX SUPERFAMILY |
| | | | | | | SIGNATURE |
| 1401 | PR00830A | 8.41 | 9.673e−13 | 174-193 | PR00830 | ENDOPEPTIDASE LA (LON) |
| | | | | | | SERINE PROTEASE (S16) |
| | | | | | | SIGNATURE |
| 1403 | BL00282 | 16.88 | 5.800e−09 | 127-149 | BL00282 | Kazal serine protease |
| | | | | | | inhibitors family |
| | | | | | | proteins. |
| 1403 | BL00612B | 11.35 | 4.600e−09 | 169-201 | BL00612 | Osteonectin domain |
| | | | | | | proteins. |
| 1404 | BL00229A | 23.57 | 3.591e−09 | 60-98 | BL00229 | Tau and MAP proteins |
| | | | | | | tubulin-binding domain |
| | | | | | | proteins. |
| 1404 | PR00456F | 5.86 | 6.400e−12 | 102-113 | PR00456 | RIBOSOMAL PROTEIN P2 |
| | | | | | | SIGNATURE |
| 1408 | BL00989A | 11.66 | 4.000e−12 | 5-18 | BL00989 | Clathrin adaptor |
| | | | | | | complexes small chain |
| | | | | | | proteins. |
| 1408 | BL00989B | 26.51 | 1.000e−40 | 66-116 | BL00989 | Clathrin adaptor |
| | | | | | | complexes small chain |
| | | | | | | proteins. |
| 1408 | PR00317F | 10.90 | 5.935e−09 | 103-117 | PR00317 | EPENDYMIN SIGNATURE |
| 1409 | BL00194 | 12.16 | 3.813e−15 | 547-559 | BL00194 | Thioredoxin family |
| | | | | | | proteins. |
| 1409 | BL00194 | 12.16 | 6.727e−17 | 198-210 | BL00194 | Thioredoxin family |
| | | | | | | proteins. |
| 1409 | BL00194 | 12.16 | 8.071e−16 | 83-95 | BL00194 | Thioredoxin family |
| | | | | | | proteins. |
| 1409 | PR00421A | 10.15 | 1.429e−10 | 546-554 | PR00421 | THIOREDOXIN FAMILY |
| | | | | | | SIGNATURE |
| 1409 | PR00421A | 10.15 | 3.143e−10 | 82-90 | PR00421 | THIOREDOXIN FAMILY |
| | | | | | | SIGNATURE |
| 1409 | PR00421A | 10.15 | 6.571e−10 | 197-205 | PR00421 | THIOREDOXIN FAMILY |
| | | | | | | SIGNATURE |
| 1409 | PR00421B | 11.40 | 1.783e−12 | 205-214 | PR00421 | THIOREDOXIN FAMILY |
| | | | | | | SIGNATURE |
| 1409 | PR00421B | 11.40 | 7.000e−14 | 90-99 | PR00421 | THIOREDOXIN FAMILY |
| | | | | | | SIGNATURE |
| 1409 | PR00421B | 11.40 | 7.750e−13 | 554-563 | PR00421 | THIOREDOXIN FAMILY |
| | | | | | | SIGNATURE |
| 1409 | PR00421C | 13.60 | 1.346e−09 | 596-607 | PR00421 | THIOREDOXIN FAMILY |
| | | | | | | SIGNATURE |
| 1409 | PR00421C | 13.60 | 7.231e−09 | 248-259 | PR00421 | THIOREDOXIN FAMILY |
| | | | | | | SIGNATURE |
| 1409 | PR00421C | 13.60 | 9.308e−09 | 133-144 | PR00421 | THIOREDOXIN FAMILY |
| | | | | | | SIGNATURE |
| 1410 | BL00567A | 10.66 | 6.459e−11 | 21-39 | BL00567 | Phosphoribulokinase |
| | | | | | | proteins. |
| 1410 | BL01294A | 5.15 | 3.872e−12 | 22-33 | BL01294 | Uncharacterized protein |
| | | | | | | family UPF0038 |
| | | | | | | proteins. |
| 1410 | BL01294B | 7.90 | 5.065e−12 | 46-60 | BL01294 | Uncharacterized protein |
| | | | | | | family UPF0038 |
| | | | | | | proteins. |
| 1410 | BL01294C | 22.06 | 5.219e−26 | 71-108 | BL01294 | Uncharacterized protein |
| | | | | | | family UPF0038 |
| | | | | | | proteins. |
| 1410 | BL01294E | 20.47 | 3.800e−23 | 157-197 | BL01294 | Uncharacterized protein |
| | | | | | | family UPF0038 |
| | | | | | | proteins. |
| 1410 | PR00988A | 6.39 | 6.276e−12 | 19-36 | PR00988 | URIDINE KINASE |
| | | | | | | SIGNATURE |
| 1411 | BL00132A | 26.07 | 3.329e−09 | 50-90 | BL00132 | zinc carboxypeptidases, |
| | | | | | | zinc-binding region 1 |
| | | | | | | proteins. |
| 1411 | PR00765A | 12.09 | 2.500e−09 | 76-88 | PR00765 | CARBOXYPEPTIDASE A |
| | | | | | | METALLOPROTEASE (M14) |
| | | | | | | FAMILY SIGNATURE |
| 1411 | PR00765B | 15.57 | 2.800e−12 | 102-116 | PR00765 | CARBOXYPEPTIDASE A |
| | | | | | | METALLOPROTEASE (M14) |
| | | | | | | FAMILY SIGNATURE |
| 1411 | PR00765C | 12.55 | 3.659e−09 | 181-189 | PR00765 | CARBOXYPEPTIDASE A |
| | | | | | | METALLOPROTEASE (M14) |
| | | | | | | FAMILY SIGNATURE |
| 1411 | PR00765D | 14.16 | 9.053e−11 | 240-253 | PR00765 | CARBOXYPEPTIDASE A |
| | | | | | | METALLOPROTEASE (M14) |
| | | | | | | FAMILY SIGNATURE |
| 1412 | BL00203 | 13.94 | 5.979e−10 | 91-136 | BL00203 | Vertebrate |
| | | | | | | metallothioneins |
| | | | | | | proteins. |
| 1412 | BL00243I | 31.77 | 2.521e−09 | 503-545 | BL00243 | Integrins beta chain |
| | | | | | | cysteine-rich domain |
| | | | | | | proteins. |
| 1412 | BL01177B | 13.61 | 6.400e−16 | 200-215 | BL01177 | Anaphylatoxin domain |
| | | | | | | proteins. |
| 1412 | BL01177C | 17.39 | 2.875e−20 | 448-466 | BL01177 | Anaphylatoxin domain |
| | | | | | | proteins. |
| 1412 | BL01177C | 17.39 | 5.286e−09 | 492-510 | BL01177 | Anaphylatoxin domain |
| | | | | | | proteins. |
| 1412 | BL01177D | 17.50 | 3.739e−17 | 474-491 | BL01177 | Anaphylatoxin domain |
| | | | | | | proteins. |
| 1412 | BL01177E | 20.64 | 1.375e−26 | 496-522 | 8L01177 | Anaphylatoxin domain |
| | | | | | | proteins. |
| 1412 | BL01187A | 9.98 | 1.000e−12 | 439-450 | BL01187 | Calcium-binding EGF- |
| | | | | | | like domain proteins |
| | | | | | | pattern proteins. |
| 1412 | BL01187A | 9.98 | 4.789e−11 | 260-271 | BL01187 | Calcium-binding EGF- |
| | | | | | | like domain proteins |
| | | | | | | pattern proteins. |
| 1412 | BL01187A | 9.98 | 4.857e−10 | 479-490 | BL01187 | Calcium-binding EGF- |
| | | | | | | like domain proteins |
| | | | | | | pattern proteins. |
| 1412 | BL01187A | 9.98 | 6.625e−09 | 397-408 | BL01187 | Calcium-binding EGF- |
| | | | | | | like domain proteins |
| | | | | | | pattern proteins. |
| 1412 | BL01187A | 9.98 | 9.571e−10 | 523-534 | BL01187 | Calcium-binding EGF- |
| | | | | | | like domain proteins |
| | | | | | | pattern proteins. |
| 1412 | BL01187A | 9.98 | 9.625e−09 | 214-225 | BL01187 | Calcium-binding EGF- |
| | | | | | | like domain proteins |
| | | | | | | pattern proteins. |
| 1412 | BL01187B | 12.04 | 3.250e−14 | 415-430 | BL01187 | Calcium-binding EGF- |
| | | | | | | like domain proteins |
| | | | | | | pattern proteins. |
| 1412 | BL01187B | 12.04 | 4.600e−10 | 190-205 | BL01187 | Calcium-binding EGF- |
| | | | | | | like domain proteins |
| | | | | | | pattern proteins. |
| 1412 | BL01187B | 12.04 | 5.333e−12 | 373-388 | BL01187 | Calcium-binding EGF- |
| | | | | | | like domain proteins |
| | | | | | | pattern proteins. |
| 1412 | BL01187B | 12.04 | 5.500e−09 | 498-513 | BL01187 | Calcium-binding EGF- |
| | | | | | | like domain proteins |
| | | | | | | pattern proteins. |
| 1412 | BL01187B | 12.04 | 6.478e−13 | 454-469 | BL01187 | Calcium-binding EGF- |
| | | | | | | like domain proteins |
| | | | | | | pattern proteins. |
| 1412 | PR00010A | 11.79 | 9.308e−09 | 441-452 | PR00010 | TYPE II EGF-LIKE |
| | | | | | | SIGNATURE |
| 1412 | PR00010C | 11.16 | 5.714e−09 | 420-430 | PR00010 | TYPE II EGF-LIKE |
| | | | | | | SIGNATURE |
| 1412 | PR00538B | 10.45 | 8.688e−09 | 108-127 | PR00538 | MUSCARINIC M1 RECEPTOR |
| | | | | | | SIGNATURE |
| 1412 | PR00907B | 11.29 | 9.766e−10 | 494-510 | PR00907 | THROMBOMODULIN |
| | | | | | | SIGNATURE |
| 1413 | BL00030A | 14.39 | 6.143e−13 | 346-364 | BL00030 | Eukaryotic RNA-binding |
| | | | | | | region RNP-1 proteins. |
| 1413 | BL00030A | 14.39 | 9.000e−10 | 260-278 | BL00030 | Eukaryotic RNA-binding |
| | | | | | | region RNP-1 proteins. |
| 1413 | BL00030B | 7.03 | 6.211e−09 | 295-304 | BL00030 | Eukaryotic RNA-binding |
| | | | | | | region RNP-1 proteins. |
| 1413 | BL00030B | 7.03 | 8.800e−10 | 382-391 | BL00030 | Eukaryotic RNA-binding |
| | | | | | | region RNP-1 proteins. |
| 1413 | BL00291A | 4.49 | 7.506e−10 | 418-452 | BL00291 | Prion protein. |
| 1413 | BL00412D | 16.54 | 7.245e−09 | 130-180 | BL00412 | Neuromodulin (GAP-43) |
| | | | | | | proteins. |
| 1414 | BL00054A | 25.71 | 9.591e−34 | 29-69 | BL00054 | Ribosomal protein S11 |
| | | | | | | proteins. |
| 1414 | BL00054B | 16.72 | 1.000e−36 | 106-147 | BL00054 | Ribosomal protein S11 |
| | | | | | | proteins. |
| 1416 | BL01199 | 8.49 | 7.923e−12 | 124-137 | BL01199 | Ribosomal protein L1 |
| | | | | | | proteins. |
| 1418 | PR00019A | 11.19 | 7.261e−10 | 256-269 | PR00019 | LEUCINE-RICH REPEAT |
| | | | | | | SIGNATURE |
| 1418 | PR00019A | 11.19 | 7.600e−12 | 186-199 | PR00019 | LEUCINE-RICH REPEAT |
| | | | | | | SIGNATURE |
| 1418 | PR00019B | 11.36 | 3.880e−09 | 253-266 | PR00019 | LEUCINE-RICH REPEAT |
| | | | | | | SIGNATURE |
| 1418 | PR00019B | 11.36 | 5.000e−11 | 183-196 | PR00019 | LEUCINE-RICH REPEAT |
| | | | | | | SIGNATURE |
| 1419 | PD00210 | 15.25 | 3.400e−15 | 112-127 | PD00210 | PROTEIN ANTIOXIDANT |
| | | | | | | PEROXIDASE RED. |
| 1421 | BL00213A | 12.95 | 4.316e−10 | 40-53 | BL00213 | Lipocalin proteins. |
| 1421 | PR00179A | 13.78 | 2.080e−10 | 40-52 | PR00179 | LIPOCALIN SIGNATURE |
| 1421 | PR00179B | 9.56 | 7.375e−12 | 120-132 | PR00179 | LIPOCALIN SIGNATURE |
| 1422 | BL01171 | 19.94 | 1.000e−40 | 30-67 | BL01171 | Ribosomal protein L21e |
| | | | | | | proteins. |
| 1423 | BL00361A | 11.94 | 9.206e−13 | 52-67 | BL00361 | Ribosomal protein S10 |
| | | | | | | proteins. |
| 1423 | BL00361B | 18.34 | 5.750e−22 | 69-91 | BL00361 | Ribosomal protein S10 |
| | | | | | | proteins. |
| 1423 | PR00971A | 13.33 | 2.432e−09 | 19-32 | PR00971 | RIBOSOMAL PROTEIN S10 |
| | | | | | | FAMILY SIGNATURE |
| 1423 | PR00971B | 11.31 | 2.742e−13 | 54-69 | PR00971 | RIBOSOMAL PROTEIN S10 |
| | | | | | | FAMILY SIGNATURE |
| 1423 | PR00971C | 11.85 | 6.906e−16 | 76-90 | PR00971 | RIBOSOMAL PROTEIN S10 |
| | | | | | | FAMILY SIGNATURE |
| 1424 | BL00050A | 23.71 | 9.250e−27 | 94-126 | BL00050 | Ribosomal protein L23 |
| | | | | | | proteins. |
| 1424 | BL00050B | 14.81 | 8.125e−12 | 133-146 | BL00050 | Ribosomal protein L23 |
| | | | | | | proteins. |
| 1424 | PR00925B | 3.73 | 3.089e−10 | 12-24 | PR00925 | NONHISTONE CHROMOSOMAL |
| | | | | | | PROTEIN HMG17 FAMILY |
| | | | | | | SIGNATURE |
| 1427 | PR00326A | 8.75 | 5.979e−14 | 245-265 | PR00326 | GTP1/OBG GTP-BINDING |
| | | | | | | PROTEIN FAMILY |
| | | | | | | SIGNATURE |
| 1427 | PR00449A | 13.20 | 9.813e−11 | 243-264 | PR00449 | TRANSFORMING PROTEIN |
| | | | | | | P21 RAS SIGNATURE |
| 1429 | PR00776A | 10.77 | 1.844e−29 | 30-54 | PR00776 | HEMOGLOBINASE (C13) |
| | | | | | | CYSTEINE PROTEASE |
| | | | | | | SIGNATURE |
| 1429 | PR00776B | 15.39 | 5.154e−30 | 55-84 | PR00776 | HEMOGLOBINASE (C13) |
| | | | | | | CYSTEINE PROTEASE |
| | | | | | | SIGNATURE |
| 1429 | PR00776C | 11.51 | 9.357e−18 | 102-117 | PR00776 | HEMOGLOBINASE (C13) |
| | | | | | | CYSTEINE PROTEASE |
| | | | | | | SIGNATURE |
| 1429 | PR00776D | 11.56 | 7.231e−22 | 131-150 | PR00776 | HEMOGLOBINASE (C13) |
| | | | | | | CYSTEINE PROTEASE |
| | | | | | | SIGNATURE |
| 1429 | PR00776E | 12.29 | 1.375e−19 | 179-195 | PR00776 | HEMOGLOBINASE (C13) |
| | | | | | | CYSTEINE PROTEASE |
| | | | | | | SIGNATURE |
| 1430 | BL00951A | 15.10 | 7.750e−39 | 2-37 | BL00951 | ER lumen protein |
| | | | | | | retaining receptor |
| | | | | | | proteins. |
| 1430 | BL00951B | 14.23 | 3.100e−31 | 38-68 | BL00951 | ER lumen protein |
| | | | | | | retaining receptor |
| | | | | | | proteins. |
| 1430 | BL00951C | 19.35 | 1.000e−40 | 93-141 | BL00951 | ER lumen protein |
| | | | | | | retaining receptor |
| | | | | | | proteins. |
| 1430 | BL00951D | 13.94 | 6.000e−38 | 142-176 | BL00951 | ER lumen protein |
| | | | | | | retaining receptor |
| | | | | | | proteins. |
| 1430 | PR00660A | 13.28 | 9.591e−21 | 4-23 | PR00660 | ER LUMEN PROTEIN |
| | | | | | | RETAINING RECEPTOR |
| | | | | | | SIGNATURE |
| 1430 | PR00660B | 13.72 | 4.000e−24 | 31-51 | PR00660 | ER LUMEN PROTEIN |
| | | | | | | RETAINING RECEPTOR |
| | | | | | | SIGNATURE |
| 1430 | PR00660C | 11.32 | 6.211e−17 | 52-68 | PR00660 | ER LUMEN PROTEIN |
| | | | | | | RETAINING RECEPTOR |
| | | | | | | SIGNATURE |
| 1430 | PR00660D | 12.01 | 7.750e−26 | 120-140 | PR00660 | ER LUMEN PROTEIN |
| | | | | | | RETAINING RECEPTOR |
| | | | | | | SIGNATURE |
| 1430 | PR00660E | 11.35 | 3.625e−16 | 142-156 | PR00660 | ER LUMEN PROTEIN |
| | | | | | | RETAINING RECEPTOR |
| | | | | | | SIGNATURE |
| 1430 | PR00660F | 12.40 | 7.818e−22 | 156-174 | PR00660 | ER LUMEN PROTEIN |
| | | | | | | RETAINING RECEPTOR |
| | | | | | | SIGNATURE |
| 1430 | PR00660G | 11.77 | 8.500e−20 | 184-199 | PR00660 | ER LUMEN PROTEIN |
| | | | | | | RETAINING RECEPTOR |
| | | | | | | SIGNATURE |
| 1431 | BL00291A | 4.49 | 7.103e−09 | 42-76 | BL00291 | Prion protein. |
| 1431 | BL00420A | 20.42 | 1.000e−08 | 1000-1028 | BL00420 | Speract receptor repeat |
| | | | | | | proteins domain |
| | | | | | | proteins. |
| 1431 | BL00420A | 20.42 | 1.000e−09 | 241-269 | BL00420 | Speract receptor repeat |
| | | | | | | proteins domain |
| | | | | | | proteins. |
| 1431 | BL00420A | 20.42 | 1.000e−09 | 538-566 | BL00420 | Speract receptor repeat |
| | | | | | | proteins domain |
| | | | | | | proteins. |
| 1431 | BL00420A | 20.42 | 1.138e−09 | 667-695 | BL00420 | Speract receptor repeat |
| | | | | | | proteins domain |
| | | | | | | proteins. |
| 1431 | BL00420A | 20.42 | 1.138e−09 | 934-962 | BL00420 | Speract receptor repeat |
| | | | | | | proteins domain |
| | | | | | | proteins. |
| 1431 | BL00420A | 20.42 | 1.415e−09 | 166-194 | BL00420 | Speract receptor repeat |
| | | | | | | proteins domain |
| | | | | | | proteins. |
| 1431 | BL00420A | 20.42 | 1.590e−10 | 514-542 | BL00420 | Speract receptor repeat |
| | | | | | | proteins domain |
| | | | | | | proteins. |
| 1431 | BL00420A | 20.42 | 1.655e−11 | 685-713 | BL00420 | Speract receptor repeat |
| | | | | | | proteins domain |
| | | | | | | proteins. |
| 1431 | BL00420A | 20.42 | 1.692e−09 | 349-377 | BL00420 | Speract receptor repeat |
| | | | | | | proteins domain |
| | | | | | | proteins. |
| 1431 | BL00420A | 20.42 | 1.692e−09 | 616-644 | BL00420 | Speract receptor repeat |
| | | | | | | proteins domain |
| | | | | | | proteins. |
| 1431 | BL00420A | 20.42 | 1.692e−09 | 631-659 | BL00420 | Speract receptor repeat |
| | | | | | | proteins domain |
| | | | | | | proteins. |
| 1431 | BL00420A | 20.42 | 1.831e−09 | 688-716 | BL00420 | Speract receptor repeat |
| | | | | | | proteins domain |
| | | | | | | proteins. |
| 1431 | BL00420A | 20.42 | 1.885e−10 | 562-590 | BL00420 | Speract receptor repeat |
| | | | | | | proteins domain |
| | | | | | | proteins. |
| 1431 | BL00420A | 20.42 | 1.969e−09 | 682-710 | BL00420 | Speract receptor repeat |
| | | | | | | proteins domain |
| | | | | | | proteins. |
| 1431 | BL00420A | 20.42 | 2.108e−09 | 817-845 | BL00420 | Speract receptor repeat |
| | | | | | | proteins domain |
| | | | | | | proteins. |
| 1431 | BL00420A | 20.42 | 2.246e−09 | 451-479 | BL00420 | Speract receptor repeat |
| | | | | | | proteins domain |
| | | | | | | proteins. |
| 1431 | BL00420A | 20.42 | 2.662e−09 | 334-362 | BL00420 | Speract receptor repeat |
| | | | | | | proteins domain |
| | | | | | | proteins. |
| 1431 | BL00420A | 20.42 | 2.800e−09 | 424-452 | BL00420 | Speract receptor repeat |
| | | | | | | proteins domain |
| | | | | | | proteins. |
| 1431 | BL00420A | 20.42 | 2.964e−11 | 400-428 | BL00420 | Speract receptor repeat |
| | | | | | | proteins domain |
| | | | | | | proteins. |
| 1431 | BL00420A | 20.42 | 2.964e−11 | 919-947 | BL00420 | Speract receptor repeat |
| | | | | | | proteins domain |
| | | | | | | proteins. |
| 1431 | BL00420A | 20.42 | 3.077e−09 | 895-923 | BL00420 | Speract receptor repeat |
| | | | | | | proteins domain |
| | | | | | | proteins. |
| 1431 | BL00420A | 20.42 | 3.077e−09 | 94-122 | BL00420 | Speract receptor repeat |
| | | | | | | proteins domain |
| | | | | | | proteins. |
| 1431 | BL00420A | 20.42 | 3.215e−09 | 478-506 | BL00420 | Speract receptor repeat |
| | | | | | | proteins domain |
| | | | | | | proteins. |
| 1431 | BL00420A | 20.42 | 3.361e−10 | 163-191 | BL00420 | Speract receptor repeat |
| | | | | | | proteins domain |
| | | | | | | proteins. |
| 1431 | BL00420A | 20.42 | 3.361e−10 | 781-809 | BL00420 | Speract receptor repeat |
| | | | | | | proteins domain |
| | | | | | | proteins. |
| 1431 | BL00420A | 20.42 | 3.423e−12 | 328-356 | BL00420 | Speract receptor repeat |
| | | | | | | proteins domain |
| | | | | | | proteins. |
| 1431 | BL00420A | 20.42 | 3.508e−10 | 853-881 | BL00420 | Speract receptor repeat |
| | | | | | | proteins domain |
| | | | | | | proteins. |
| 1431 | BL00420A | 20.42 | 3.631e−09 | 304-332 | BL00420 | Speract receptor repeat |
| | | | | | | proteins domain |
| | | | | | | proteins. |
| 1431 | BL00420A | 20.42 | 3.769e−09 | 712-740 | BL00420 | Speract receptor repeat |
| | | | | | | proteins domain |
| | | | | | | proteins. |
| 1431 | BL00420A | 20.42 | 3.908e−09 | 487-515 | BL00420 | Speract receptor repeat |
| | | | | | | proteins domain |
| | | | | | | proteins. |
| 1431 | BL00420A | 20.42 | 4.323e−09 | 751-779 | BL00420 | Speract receptor repeat |
| | | | | | | proteins domain |
| | | | | | | proteins. |
| 1431 | BL00420A | 20.42 | 4.462e−09 | 640-668 | BL00420 | Speract receptor repeat |
| | | | | | | proteins domain |
| | | | | | | proteins. |
| 1431 | BL00420A | 20.42 | 4.541e−10 | 115-143 | BL00420 | Speract receptor repeat |
| | | | | | | proteins domain |
| | | | | | | proteins. |
| 1431 | BL00420A | 20.42 | 4.738e−09 | 415-443 | BL00420 | Speract receptor repeat |
| | | | | | | proteins domain |
| | | | | | | proteins. |
| 1431 | BL00420A | 20.42 | 4.877e−09 | 190-218 | BL00420 | Speract receptor repeat |
| | | | | | | proteins domain |
| | | | | | | proteins. |
| 1431 | BL00420A | 20.42 | 5.015e−09 | 319-347 | BL00420 | Speract receptor repeat |
| | | | | | | proteins domain |
| | | | | | | proteins. |
| 1431 | BL00420A | 20.42 | 5.091e−11 | 787-815 | BL00420 | Speract receptor repeat |
| | | | | | | proteins domain |
| | | | | | | proteins. |
| 1431 | BL00420A | 20.42 | 5.569e−09 | 322-350 | BL00420 | Speract receptor repeat |
| | | | | | | proteins domain |
| | | | | | | proteins. |
| 1431 | BL00420A | 20.42 | 5.582e−11 | 550-578 | BL00420 | Speract receptor repeat |
| | | | | | | proteins domain |
| | | | | | | proteins. |
| 1431 | BL00420A | 20.42 | 5.846e−09 | 244-272 | BL00420 | Speract receptor repeat |
| | | | | | | proteins domain |
| | | | | | | proteins. |
| 1431 | BL00420A | 20.42 | 6.016e−10 | 172-200 | BL00420 | Speract receptor repeat |
| | | | | | | proteins domain |
| | | | | | | proteins. |
| 1431 | BL00420A | 20.42 | 6.073e−11 | 208-236 | BL00420 | Speract receptor repeat |
| | | | | | | proteins domain |
| | | | | | | proteins. |
| 1431 | BL00420A | 20.42 | 6.400e−09 | 226-254 | BL00420 | Speract receptor repeat |
| | | | | | | proteins domain |
| | | | | | | proteins. |
| 1431 | BL00420A | 20.42 | 6.538e−09 | 448-476 | BL00420 | Speract receptor repeat |
| | | | | | | proteins domain |
| | | | | | | proteins. |
| 1431 | BL00420A | 20.42 | 6.607e−10 | 193-221 | BL00420 | Speract receptor repeat |
| | | | | | | proteins domain |
| | | | | | | proteins. |
| 1431 | BL00420A | 20.42 | 6.607e−10 | 628-656 | BL00420 | Speract receptor repeat |
| | | | | | | proteins domain |
| | | | | | | proteins. |
| 1431 | BL00420A | 20.42 | 6.677e−09 | 136-164 | BL00420 | Speract receptor repeat |
| | | | | | | proteins domain |
| | | | | | | proteins. |
| 1431 | BL00420A | 20.42 | 6.727e−11 | 256-284 | BL00420 | Speract receptor repeat |
| | | | | | | proteins domain |
| | | | | | | proteins. |
| 1431 | BL00420A | 20.42 | 6.754e−10 | 778-806 | BL00420 | Speract receptor repeat |
| | | | | | | proteins domain |
| | | | | | | proteins. |
| 1431 | BL00420A | 20.42 | 6.902e−10 | 901-929 | BL00420 | Speract receptor repeat |
| | | | | | | proteins domain |
| | | | | | | proteins. |
| 1431 | BL00420A | 20.42 | 7.369e−09 | 301-329 | BL00420 | Speract receptor repeat |
| | | | | | | proteins domain |
| | | | | | | proteins. |
| 1431 | BL00420A | 20.42 | 7.369e−09 | 493-521 | BL00420 | Speract receptor repeat |
| | | | | | | proteins domain |
| | | | | | | proteins. |
| 1431 | BL00420A | 20.42 | 7.508e−09 | 154-182 | BL00420 | Speract receptor repeat |
| | | | | | | proteins domain |
| | | | | | | proteins. |
| 1431 | BL00420A | 20.42 | 7.646e−09 | 439-467 | BL00420 | Speract receptor repeat |
| | | | | | | proteins domain |
| | | | | | | proteins. |
| 1431 | BL00420A | 20.42 | 7.646e−09 | 952-980 | BL00420 | Speract receptor repeat |
| | | | | | | proteins domain |
| | | | | | | proteins. |
| 1431 | BL00420A | 20.42 | 7.750e−12 | 460-488 | BL00420 | Speract receptor repeat |
| | | | | | | proteins domain |
| | | | | | | proteins. |
| 1431 | BL00420A | 20.42 | 7.873e−11 | 583-611 | BL00420 | Speract receptor repeat |
| | | | | | | proteins domain |
| | | | | | | proteins. |
| 1431 | BL00420A | 20.42 | 7.923e−09 | 48-76 | BL00420 | Speract receptor repeat |
| | | | | | | proteins domain |
| | | | | | | proteins. |
| 1431 | BL00420A | 20.42 | 7.923e−09 | 865-893 | BL00420 | Speract receptor repeat |
| | | | | | | proteins domain |
| | | | | | | proteins. |
| 1431 | BL00420A | 20.42 | 7.923e−09 | 922-950 | BL00420 | Speract receptor repeat |
| | | | | | | proteins domain |
| | | | | | | proteins. |
| 1431 | BL00420A | 20.42 | 8.062e−09 | 283-311 | BL00420 | Speract receptor repeat |
| | | | | | | proteins domain |
| | | | | | | proteins. |
| 1431 | BL00420A | 20.42 | 8.062e−09 | 637-665 | BL00420 | Speract receptor repeat |
| | | | | | | proteins domain |
| | | | | | | proteins. |
| 1431 | BL00420A | 20.42 | 8.062e−09 | 838-866 | BL00420 | Speract receptor repeat |
| | | | | | | proteins domain |
| | | | | | | proteins. |
| 1431 | BL00420A | 20.42 | 8.200e−11 | 595-623 | BL00420 | Speract receptor repeat |
| | | | | | | proteins domain |
| | | | | | | proteins. |
| 1431 | BL00420A | 20.42 | 8.377e−10 | 313-341 | BL00420 | Speract receptor repeat |
| | | | | | | proteins domain |
| | | | | | | proteins. |
| 1431 | BL00420A | 20.42 | 8.377e−10 | 397-425 | BL00420 | Speract receptor repeat |
| | | | | | | proteins domain |
| | | | | | | proteins. |
| 1431 | BL00420A | 20.42 | 8.377e−10 | 721-749 | BL00420 | Speract receptor repeat |
| | | | | | | proteins domain |
| | | | | | | proteins. |
| 1431 | BL00420A | 20.42 | 8.525e−10 | 517-545 | BL00420 | Speract receptor repeat |
| | | | | | | proteins domain |
| | | | | | | proteins. |
| 1431 | BL00420A | 20.42 | 8.615e−09 | 352-380 | BL00420 | Speract receptor repeat |
| | | | | | | proteins domain |
| | | | | | | proteins. |
| 1431 | BL00420A | 20.42 | 8.615e−09 | 406-434 | BL00420 | Speract receptor repeat |
| | | | | | | proteins domain |
| | | | | | | proteins. |
| 1431 | BL00420A | 20.42 | 8.691e−11 | 127-155 | BL00420 | Speract receptor repeat |
| | | | | | | proteins domain |
| | | | | | | proteins. |
| 1431 | BL00420A | 20.42 | 8.754e−09 | 373-401 | BL00420 | Speract receptor repeat |
| | | | | | | proteins domain |
| | | | | | | proteins. |
| 1431 | BL00420A | 20.42 | 8.754e−09 | 457-485 | BL00420 | Speract receptor repeat |
| | | | | | | proteins domain |
| | | | | | | proteins. |
| 1431 | BL00420A | 20.42 | 8.754e−09 | 475-503 | BL00420 | Speract receptor repeat |
| | | | | | | proteins domain |
| | | | | | | proteins. |
| 1431 | BL00420A | 20.42 | 8.820e−10 | 331-359 | BL00420 | Speract receptor repeat |
| | | | | | | proteins domain |
| | | | | | | proteins. |
| 1431 | BL00420A | 20.42 | 8.855e−11 | 913-941 | BL00420 | Speract receptor repeat |
| | | | | | | proteins domain |
| | | | | | | proteins. |
| 1431 | BL00420A | 20.42 | 8.892e−09 | 292-320 | BL00420 | Speract receptor repeat |
| | | | | | | proteins domain |
| | | | | | | proteins. |
| 1431 | BL00420A | 20.42 | 9.031e−09 | 427-455 | BL00420 | Speract receptor repeat |
| | | | | | | proteins domain |
| | | | | | | proteins. |
| 1431 | BL00420A | 20.42 | 9.031e−09 | 565-593 | BL00420 | Speract receptor repeat |
| | | | | | | proteins domain |
| | | | | | | proteins. |
| 1431 | BL00420A | 20.42 | 9.031e−09 | 910-938 | BL00420 | Speract receptor repeat |
| | | | | | | proteins domain |
| | | | | | | proteins. |
| 1431 | BL00420A | 20.42 | 9.262e−10 | 253-281 | BL00420 | Speract receptor repeat |
| | | | | | | proteins domain |
| | | | | | | proteins. |
| 1431 | BL00420A | 20.42 | 9.446e−09 | 856-884 | BL00420 | Speract receptor repeat |
| | | | | | | proteins domain |
| | | | | | | proteins. |
| 1431 | BL00420A | 20.42 | 9.509e−11 | 100-128 | BL00420 | Speract receptor repeat |
| | | | | | | proteins domain |
| | | | | | | proteins. |
| 1431 | BL00420A | 20.42 | 9.509e−11 | 874-902 | BL00420 | Speract receptor repeat |
| | | | | | | proteins domain |
| | | | | | | proteins. |
| 1431 | BL00420A | 20.42 | 9.585e−09 | 799-827 | BL00420 | Speract receptor repeat |
| | | | | | | proteins domain |
| | | | | | | proteins. |
| 1431 | BL00420A | 20.42 | 9.723e−09 | 376-404 | BL00420 | Speract receptor repeat |
| | | | | | | proteins domain |
| | | | | | | proteins. |
| 1431 | BL00420A | 20.42 | 9.723e−09 | 481-509 | BL00420 | Speract receptor repeat |
| | | | | | | proteins domain |
| | | | | | | proteins. |
| 1431 | BL00420A | 20.42 | 9.852e−10 | 106-134 | BL00420 | Speract receptor repeat |
| | | | | | | proteins domain |
| | | | | | | proteins. |
| 1431 | BL00420A | 20.42 | 9.852e−10 | 238-266 | BL00420 | Speract receptor repeat |
| | | | | | | proteins domain |
| | | | | | | proteins. |
| 1431 | BL00420A | 20.42 | 9.852e−10 | 91-119 | BL00420 | Speract receptor repeat |
| | | | | | | proteins domain |
| | | | | | | proteins. |
| 1431 | BL01113A | 17.99 | 1.000e−08 | 397-423 | BL01113 | C1q domain proteins. |
| 1431 | BL01113A | 17.99 | 1.000e−09 | 898-924 | BL01113 | C1q domain proteins. |
| 1431 | BL01113A | 17.99 | 1.173e−09 | 145-171 | BL01113 | C1q domain proteins. |
| 1431 | BL01113A | 17.99 | 1.243e−13 | 313-339 | BL01113 | C1q domain proteins. |
| 1431 | BL01113A | 17.99 | 1.519e−09 | 418-444 | BL01113 | C1q domain proteins. |
| 1431 | BL01113A | 17.99 | 1.692e−09 | 136-162 | BL01113 | C1q domain proteins. |
| 1431 | BL01113A | 17.99 | 1.923e−12 | 493-519 | BL01113 | C1q domain proteins. |
| 1431 | BL01113A | 17.99 | 2.227e−11 | 391-417 | BL01113 | C1q domain proteins. |
| 1431 | BL01113A | 17.99 | 2.340e−10 | 39-65 | BL01113 | C1q domain proteins. |
| 1431 | BL01113A | 17.99 | 2.385e−09 | 1009-1035 | BL01113 | C1q domain proteins. |
| 1431 | BL01113A | 17.99 | 2.385e−09 | 910-936 | BL01113 | C1q domain proteins. |
| 1431 | BL01113A | 17.99 | 2.532e−10 | 304-330 | BL01113 | C1q domain proteins. |
| 1431 | BL01113A | 17.99 | 2.558e−09 | 289-315 | BL01113 | C1q domain proteins. |
| 1431 | BL01113A | 17.99 | 2.846e−12 | 45-71 | BL01113 | C1q domain proteins. |
| 1431 | BL01113A | 17.99 | 3.077e−12 | 190-216 | BL01113 | C1q domain proteins. |
| 1431 | BL01113A | 17.99 | 3.250e−09 | 382-408 | BL01113 | C1q domain proteins. |
| 1431 | BL01113A | 17.99 | 3.250e−09 | 478-504 | BL01113 | C1q domain proteins. |
| 1431 | BL01113A | 17.99 | 3.423e−09 | 51-77 | BL01113 | C1q domain proteins. |
| 1431 | BL01113A | 17.99 | 3.423e−09 | 799-825 | BL01113 | C1q domain proteins. |
| 1431 | BL01113A | 17.99 | 3.769e−09 | 808-834 | BL01113 | C1q domain proteins. |
| 1431 | BL01113A | 17.99 | 3.769e−09 | 94-120 | BL01113 | C1q domain proteins. |
| 1431 | BL01113A | 17.99 | 4.064e−10 | 127-153 | BL01113 | C1q domain proteins. |
| 1431 | BL01113A | 17.99 | 4.068e−11 | 48-74 | BL01113 | C1q domain proteins. |
| 1431 | BL01113A | 17.99 | 4.115e−09 | 634-660 | BL01113 | C1q domain proteins. |
| 1431 | BL01113A | 17.99 | 4.255e−10 | 460-486 | BL01113 | C1q domain proteins. |
| 1431 | BL01113A | 17.99 | 4.273e−11 | 208-234 | BL01113 | C1q domain proteins. |
| 1431 | BL01113A | 17.99 | 4.288e−09 | 172-198 | BL01113 | C1q domain proteins. |
| 1431 | BL01113A | 17.99 | 4.447e−10 | 295-321 | BL01113 | C1q domain proteins. |
| 1431 | BL01113A | 17.99 | 4.447e−10 | 781-807 | BL01113 | C1q domain proteins. |
| 1431 | BL01113A | 17.99 | 4.447e−10 | 922-948 | BL01113 | C1q domain proteins. |
| 1431 | BL01113A | 17.99 | 4.462e−09 | 433-459 | BL01113 | C1q domain proteins. |
| 1431 | BL01113A | 17.99 | 4.462e−09 | 805-831 | BL01113 | C1q domain proteins. |
| 1431 | BL01113A | 17.99 | 4.635e−09 | 121-147 | BL01113 | C1q domain proteins. |
| 1431 | BL01113A | 17.99 | 4.682e−11 | 286-312 | BL01113 | C1q domain proteins. |
| 1431 | BL01113A | 17.99 | 4.808e−09 | 250-276 | BL01113 | C1q domain proteins. |
| 1431 | BL01113A | 17.99 | 4.808e−09 | 445-471 | BL01113 | C1q domain proteins. |
| 1431 | BL01113A | 17.99 | 4.981e−09 | 163-189 | BL01113 | C1q domain proteins. |
| 1431 | BL01113A | 17.99 | 4.981e−09 | 166-192 | BL01113 | C1q domain proteins. |
| 1431 | BL01113A | 17.99 | 4.981e−09 | 667-693 | BL01113 | C1q domain proteins. |
| 1431 | BL01113A | 17.99 | 5.021e−10 | 1000-1026 | BL01113 | C1q domain proteins. |
| 1431 | BL01113A | 17.99 | 5.021e−10 | 520-546 | BL01113 | C1q domain proteins. |
| 1431 | BL01113A | 17.99 | 5.091e−11 | 97-123 | BL01113 | C1q domain proteins. |
| 1431 | BL01113A | 17.99 | 5.327e−09 | 151-177 | BL01113 | C1q domain proteins. |
| 1431 | BL01113A | 17.99 | 5.327e−09 | 352-378 | BL01113 | C1q domain proteins. |
| 1431 | BL01113A | 17.99 | 5.404e−10 | 184-210 | BL01113 | C1q domain proteins. |
| 1431 | BL01113A | 17.99 | 5.596e−10 | 1081-1107 | BL01113 | C1q domain proteins. |
| 1431 | BL01113A | 17.99 | 5.596e−10 | 469-495 | BL01113 | C1q domain proteins. |
| 1431 | BL01113A | 17.99 | 5.596e−10 | 856-882 | BL01113 | C1q domain proteins. |
| 1431 | BL01113A | 17.99 | 5.615e−12 | 640-666 | BL01113 | C1q domain proteins. |
| 1431 | BL01113A | 17.99 | 5.673e−09 | 148-174 | BL01113 | C1q domain proteins. |
| 1431 | BL01113A | 17.99 | 5.673e−09 | 865-891 | BL01113 | C1q domain proteins. |
| 1431 | BL01113A | 17.99 | 5.705e−11 | 106-132 | BL01113 | C1q domain proteins. |
| 1431 | BL01113A | 17.99 | 5.865e−13 | 193-219 | BL01113 | C1q domain proteins. |
| 1431 | BL01113A | 17.99 | 5.979e−10 | 1054-1080 | BL01113 | C1q domain proteins. |
| 1431 | BL01113A | 17.99 | 6.019e−09 | 847-873 | BL01113 | C1q domain proteins. |
| 1431 | BL01113A | 17.99 | 6.192e−09 | 142-168 | BL01113 | C1q domain proteins. |
| 1431 | BL01113A | 17.99 | 6.192e−09 | 310-336 | BL01113 | C1q domain proteins. |
| 1431 | BL01113A | 17.99 | 6.365e−09 | 583-609 | BL01113 | C1q domain proteins. |
| 1431 | BL01113A | 17.99 | 6.365e−09 | 850-876 | BL01113 | C1q domain proteins. |
| 1431 | BL01113A | 17.99 | 6.538e−09 | 541-567 | BL01113 | C1q domain proteins. |
| 1431 | BL01113A | 17.99 | 6.538e−09 | 57-83 | BL01113 | C1q domain proteins. |
| 1431 | BL01113A | 17.99 | 6.538e−12 | 547-573 | BL01113 | C1q domain proteins. |
| 1431 | BL01113A | 17.99 | 6.538e−12 | 796-822 | BL01113 | C1q domain proteins. |
| 1431 | BL01113A | 17.99 | 6.712e−09 | 370-396 | BL01113 | C1q domain proteins. |
| 1431 | BL01113A | 17.99 | 6.712e−09 | 853-879 | BL01113 | C1q domain proteins. |
| 1431 | BL01113A | 17.99 | 6.745e−10 | 631-657 | BL01113 | C1q domain proteins. |
| 1431 | BL01113A | 17.99 | 6.885e−09 | 256-282 | BL01113 | C1q domain proteins. |
| 1431 | BL01113A | 17.99 | 6.936e−10 | 199-225 | BL01113 | C1q domain proteins. |
| 1431 | BL01113A | 17.99 | 6.936e−10 | 337-363 | BL01113 | C1q domain proteins. |
| 1431 | BL01113A | 17.99 | 7.000e−12 | 1090-1116 | BL01113 | C1q domain proteins. |
| 1431 | BL01113A | 17.99 | 7.058e−09 | 322-348 | BL01113 | C1q domain proteins. |
| 1431 | BL01113A | 17.99 | 7.128e−10 | 139-165 | BL01113 | C1q domain proteins. |
| 1431 | BL01113A | 17.99 | 7.319e−10 | 328-354 | BL01113 | C1q domain proteins. |
| 1431 | BL01113A | 17.99 | 7.319e−10 | 874-900 | BL01113 | C1q domain proteins. |
| 1431 | BL01113A | 17.99 | 7.511e−10 | 658-684 | BL01113 | C1q domain proteins. |
| 1431 | BL01113A | 17.99 | 7.750e−09 | 100-126 | BL01113 | C1q domain proteins. |
| 1431 | BL01113A | 17.99 | 7.750e−09 | 472-498 | BL01113 | C1q domain proteins. |
| 1431 | BL01113A | 17.99 | 7.750e−11 | 451-477 | BL01113 | C1q domain proteins. |
| 1431 | BL01113A | 17.99 | 7.923e−12 | 892-918 | BL01113 | C1q domain proteins. |
| 1431 | BL01113A | 17.99 | 8.054e−13 | 649-675 | BL01113 | C1q domain proteins. |
| 1431 | BL01113A | 17.99 | 8.096e−09 | 793-819 | BL01113 | C1q domain proteins. |
| 1431 | BL01113A | 17.99 | 8.269e−09 | 532-558 | BL01113 | C1q domain proteins. |
| 1431 | BL01113A | 17.99 | 8.468e−10 | 130-156 | BL01113 | C1q domain proteins. |
| 1431 | BL01113A | 17.99 | 8.773e−11 | 904-930 | BL01113 | C1q domain proteins. |
| 1431 | BL01113A | 17.99 | 8.788e−09 | 394-420 | BL01113 | C1q domain proteins. |
| 1431 | BL01113A | 17.99 | 8.851e−10 | 925-951 | BL01113 | C1q domain proteins. |
| 1431 | BL01113A | 17.99 | 8.962e−09 | 118-144 | BL01113 | C1q domain proteins. |
| 1431 | BL01113A | 17.99 | 8.977e−11 | 613-639 | BL01113 | C1q domain proteins. |
| 1431 | BL01113A | 17.99 | 9.043e−10 | 556-582 | BL01113 | C1q domain proteins. |
| 1431 | BL01113A | 17.99 | 9.077e−12 | 919-945 | BL01113 | C1q domain proteins. |
| 1431 | BL01113A | 17.99 | 9.135e−09 | 1087-1113 | BL01113 | C1q domain proteins. |
| 1431 | BL01113A | 17.99 | 9.135e−09 | 175-201 | BL01113 | C1q domain proteins. |
| 1431 | BL01113A | 17.99 | 9.135e−09 | 901-927 | BL01113 | C1q domain proteins. |
| 1431 | BL01113A | 17.99 | 9.182e−11 | 868-894 | BL01113 | C1q domain proteins. |
| 1431 | BL01113A | 17.99 | 9.234e−10 | 787-813 | BL01113 | C1q domain proteins. |
| 1431 | BL01113A | 17.99 | 9.308e−09 | 346-372 | BL01113 | C1q domain proteins. |
| 1431 | BL01113A | 17.99 | 9.308e−09 | 928-954 | BL01113 | C1q domain proteins. |
| 1431 | BL01113A | 17.99 | 9.308e−12 | 883-909 | BL01113 | C1q domain proteins. |
| 1431 | BL01113A | 17.99 | 9.426e−10 | 319-345 | BL01113 | C1q domain proteins. |
| 1431 | BL01113A | 17.99 | 9.591e−11 | 859-885 | BL01113 | C1q domain proteins. |
| 1431 | BL01113A | 17.99 | 9.795e−11 | 484-510 | BL01113 | C1q domain proteins. |
| 1431 | BL01113A | 17.99 | 9.809e−10 | 406-432 | BL01113 | C1q domain proteins. |
| 1431 | BL01113A | 17.99 | 9.827e−09 | 241-267 | BL01113 | C1q domain proteins. |
| 1431 | DM01418A | 20.83 | 1.000e−40 | 1156-1203 | DM01418 | 352 FIBRILLAR COLLAGEN |
| | | | | | | CARBOXYL-TERMINAL. |
| 1431 | DM01418B | 22.51 | 1.000e−40 | 1253-1294 | DM01418 | 352 FIBRILLAR COLLAGEN |
| | | | | | | CARBOXYL-TERMINAL. |
| 1431 | DM01418C | 20.48 | 6.276e−39 | 1324-1365 | DM01418 | 352 FIBRILLAR COLLAGEN |
| | | | | | | CARBOXYL-TERMINAL. |
| 1431 | PR00049D | 0.00 | 1.571e−10 | 1086-1100 | PR00049 | WILM'S TUMOUR PROTEIN |
| | | | | | | SIGNATURE |
| 1431 | PR00049D | 0.00 | 9.571e−10 | 56-70 | PR00049 | WILM'S TUMOUR PROTEIN |
| | | | | | | SIGNATURE |
| 1433 | BL00259 | 9.80 | 9.400e−14 | 93-103 | BL00259 | Gastrin/ |
| | | | | | | cholecystokinin family |
| | | | | | | proteins. |
| 1433 | PD01115A | 12.27 | 9.750e−12 | 1-23 | PD01115 | PRECURSOR AMPHIBIAN |
| | | | | | | SKIN SIGNAL. |
| 1435 | PR00019B | 11.36 | 5.950e−10 | 179-192 | PR00019 | LEUCINE-RICH REPEAT |
| | | | | | | SIGNATURE |
| 1437 | BL00141A | 12.10 | 2.636e−19 | 110-125 | BL00141 | Eukaryotic and viral |
| | | | | | | aspartyl proteases |
| | | | | | | proteins. |
| 1437 | BL00141B | 12.14 | 7.231e−11 | 214-225 | BL00141 | Eukaryotic and viral |
| | | | | | | aspartyl proteases |
| | | | | | | proteins. |
| 1437 | BL00141C | 9.74 | 9.471e−09 | 266-275 | BL00141 | Eukaryotic and viral |
| | | | | | | aspartyl proteases |
| | | | | | | proteins. |
| 1437 | BL00141D | 6.28 | 5.154e−10 | 310-319 | BL00141 | Eukaryotic and viral |
| | | | | | | aspartyl proteases |
| | | | | | | proteins. |
| 1437 | BL00141E | 14.32 | 1.900e−23 | 402-425 | BL00141 | Eukaryotic and viral |
| | | | | | | aspartyl proteases |
| | | | | | | proteins. |
| 1437 | PR00792A | 11.54 | 4.273e−24 | 103-123 | PR00792 | PEPSIN (A1) ASPARTIC |
| | | | | | | PROTEASE FAMILY |
| | | | | | | SIGNATURE |
| 1437 | PR00792B | 12.78 | 4.682e−11 | 261-274 | PR00792 | PEPSIN (A1) ASPARTIC |
| | | | | | | PROTEASE FAMILY |
| | | | | | | SIGNATURE |
| 1437 | PR00792C | 9.10 | 1.000e−11 | 310-321 | PR00792 | PEPSIN (A1) ASPARTIC |
| | | | | | | PROTEASE FAMILY |
| | | | | | | SIGNATURE |
| 1437 | PR00792D | 12.74 | 5.000e−17 | 401-416 | PR00792 | PEPSIN (A1) ASPARTIC |
| | | | | | | PROTEASE FAMILY |
| | | | | | | SIGNATURE |
| 1438 | BL00420A | 20.42 | 1.000e−08 | 48-76 | BL00420 | Speract receptor repeat |
| | | | | | | proteins domain |
| | | | | | | proteins. |
| 1438 | BL00420A | 20.42 | 1.415e−09 | 72-100 | BL00420 | Speract receptor repeat |
| | | | | | | proteins domain |
| | | | | | | proteins. |
| 1438 | BL00420A | 20.42 | 1.590e−10 | 75-103 | BL00420 | Speract receptor repeat |
| | | | | | | proteins domain |
| | | | | | | proteins. |
| 1438 | BL00420A | 20.42 | 2.180e−10 | 45-73 | BL00420 | Speract receptor repeat |
| | | | | | | proteins domain |
| | | | | | | proteins. |
| 1438 | BL00420A | 20.42 | 4.109e−11 | 33-61 | BL00420 | Speract receptor repeat |
| | | | | | | proteins domain |
| | | | | | | proteins. |
| 1438 | BL00420A | 20.42 | 7.055e−11 | 84-112 | BL00420 | Speract receptor repeat |
| | | | | | | proteins domain |
| | | | | | | proteins. |
| 1438 | BL00420A | 20.42 | 8.082e−10 | 36-64 | BL00420 | Speract receptor repeat |
| | | | | | | proteins domain |
| | | | | | | proteins. |
| 1438 | BL00420A | 20.42 | 9.410e−10 | 87-115 | BL00420 | Speract receptor repeat |
| | | | | | | proteins domain |
| | | | | | | proteins. |
| 1438 | BL01113A | 17.99 | 1.000e−13 | 45-71 | BL01113 | C1q domain proteins. |
| 1438 | BL01113A | 17.99 | 1.519e−09 | 78-104 | BL01113 | C1q domain proteins. |
| 1438 | BL01113A | 17.99 | 4.405e−13 | 54-80 | BL01113 | C1q domain proteins. |
| 1438 | BL01113A | 17.99 | 5.050e−21 | 36-62 | BL01113 | C1q domain proteins. |
| 1438 | BL01113A | 17.99 | 5.154e−12 | 81-107 | BL01113 | C1q domain proteins. |
| 1438 | BL01113A | 17.99 | 5.655e−16 | 33-59 | BL01113 | C1q domain proteins. |
| 1438 | BL01113A | 17.99 | 6.538e−09 | 30-56 | BL01113 | C1q domain proteins. |
| 1438 | BL01113A | 17.99 | 7.818e−14 | 39-65 | BL01113 | C1q domain proteins. |
| 1438 | BL01113A | 17.99 | 8.085e−10 | 75-101 | BL01113 | C1q domain proteins. |
| 1438 | BL01113A | 17.99 | 8.154e−12 | 42-68 | BL01113 | C1q domain proteins. |
| 1438 | BL01113A | 17.99 | 8.851e−10 | 84-110 | BL01113 | C1q domain proteins. |
| 1438 | BL01113A | 17.99 | 9.481e−09 | 90-116 | BL01113 | C1q domain proteins. |
| 1438 | BL01113B | 18.26 | 6.400e−33 | 139-174 | BL01113 | C1q domain proteins. |
| 1438 | BL01113C | 13.18 | 1.462e−15 | 206-225 | BL01113 | C1q domain proteins. |
| 1438 | BL01113D | 7.47 | 1.000e−11 | 240-249 | BL01113 | C1q domain proteins. |
| 1438 | PR00007A | 19.33 | 4.938e−19 | 133-159 | PR00007 | COMPLEMENT C1Q DOMAIN |
| | | | | | | SIGNATURE |
| 1438 | PR00007B | 14.16 | 7.429e−20 | 160-179 | PR00007 | COMPLEMENT C1Q DOMAIN |
| | | | | | | SIGNATURE |
| 1438 | PR00007C | 15.60 | 1.225e−15 | 206-227 | PR00007 | COMPLEMENT C1Q DOMAIN |
| | | | | | | SIGNATURE |
| 1438 | PR00007D | 9.64 | 6.885e−11 | 238-248 | PR00007 | COMPLEMENT C1Q DOMAIN |
| | | | | | | SIGNATURE |
| 1439 | BL01115A | 10.22 | 6.786e−10 | 6-49 | BL01115 | GTP-binding nuclear |
| | | | | | | protein ran proteins. |
| 1439 | PR00449A | 13.20 | 3.045e−17 | 6-27 | PR00449 | TRANSFORMING PROTEIN |
| | | | | | | P21 RAS SIGNATURE |
| 1439 | PR00449B | 14.34 | 1.000e−10 | 29-45 | PR00449 | TRANSFORMING PROTEIN |
| | | | | | | P21 RAS SIGNATURE |
| 1439 | PR00449C | 17.27 | 2.895e−18 | 46-68 | PR00449 | TRANSFORMING PROTEIN |
| | | | | | | P21 RAS SIGNATURE |
| 1439 | PR00449D | 10.79 | 2.800e−15 | 108-121 | PR00449 | TRANSFORMING PROTEIN |
| | | | | | | P21 RAS SIGNATURE |
| 1439 | PR00449E | 13.50 | 4.240e−14 | 156-178 | PR00449 | TRANSFORMING PROTEIN |
| | | | | | | P21 RAS SIGNATURE |
| 1440 | PR00492A | 11.92 | 5.714e−18 | 57-72 | PR00492 | RHO PROTEIN GDP |
| | | | | | | DISSOCIATION INHIBITOR |
| | | | | | | SIGNATURE |
| 1440 | PR00492B | 9.77 | 6.684e−23 | 73-91 | PR00492 | RHO PROTEIN GDP |
| | | | | | | DISSOCIATION INHIBITOR |
| | | | | | | SIGNATURE |
| 1440 | PR00492C | 9.68 | 7.107e−21 | 119-135 | PR00492 | RHO PROTEIN GDP |
| | | | | | | DISSOCIATION INHIBITOR |
| | | | | | | SIGNATURE |
| 1440 | PR00492D | 14.82 | 2.350e−19 | 136-151 | PR00492 | RHO PROTEIN GDP |
| | | | | | | DISSOCIATION INHIBITOR |
| | | | | | | SIGNATURE |
| 1440 | PR00492E | 13.98 | 1.000e−16 | 152-164 | PR00492 | RHO PROTEIN GDP |
| | | | | | | DISSOCIATION INHIBITOR |
| | | | | | | SIGNATURE |
| 1440 | PR00492F | 13.70 | 7.943e−24 | 178-195 | PR00492 | RHO PROTEIN GDP |
| | | | | | | DISSOCIATION INHIBITOR |
| | | | | | | SIGNATURE |
| 1441 | BL00224A | 7.42 | 4.600e−25 | 26-47 | BL00224 | Clathrin light chain |
| | | | | | | proteins. |
| 1441 | BL00224B | 16.94 | 1.000e−40 | 106-158 | BL00224 | Clathrin light chain |
| | | | | | | proteins. |
| 1441 | BL00224C | 16.60 | 1.000e−40 | 163-216 | BL00224 | Clathrin light chain |
| | | | | | | proteins. |
| 1442 | PR00759B | 11.26 | 8.385e−09 | 74-84 | PR00759 | BASIC PROTEASE (KUNITZ- |
| | | | | | | TYPE) INHIBITOR FAMILY |
| | | | | | | SIGNATURE |
| 1443 | BL00353A | 9.60 | 1.000e−40 | 2-50 | BL00353 | HMG1/2 proteins. |
| 1443 | BL00353A | 9.60 | 5.661e−11 | 3-51 | BL00353 | HMG1/2 proteins. |
| 1443 | BL00353B | 11.47 | 1.000e−40 | 78-127 | BL00353 | HMG1/2 proteins. |
| 1443 | BL00353C | 14.83 | 1.000e−40 | 128-174 | BL00353 | HMG1/2 proteins. |
| 1443 | BL00412D | 16.54 | 3.939e−09 | 165-215 | BL00412 | Neuromodulin (GAP-43) |
| | | | | | | proteins. |
| 1443 | BL00412D | 16.54 | 4.620e−10 | 160-210 | BL00412 | Neuromodulin (GAP-43) |
| | | | | | | proteins. |
| 1443 | BL00412D | 16.54 | 5.776e−09 | 162-212 | BL00412 | Neuromodulin (GAP-43) |
| | | | | | | proteins. |
| 1443 | BL00412D | 16.54 | 6.283e−10 | 161-211 | BL00412 | Neuromodulin (GAP-43) |
| | | | | | | proteins. |
| 1443 | BL00412D | 16.54 | 7.061e−09 | 159-209 | BL00412 | Neuromodulin (GAP-43) |
| | | | | | | proteins. |
| 1443 | BL00412D | 16.54 | 7.648e−11 | 164-214 | BL00412 | Neuromodulin (GAP-43) |
| | | | | | | proteins. |
| 1443 | PR00886A | 10.08 | 1.643e−30 | 40-62 | PR00886 | HIGH MOBILITY GROUP |
| | | | | | | (HMG1/HMG2) PROTEIN |
| | | | | | | SIGNATURE |
| 1443 | PR00886B | 9.88 | 3.250e−26 | 63-83 | PR00886 | HIGH MOBILITY GROUP |
| | | | | | | (HMG1/HMG2) PROTEIN |
| | | | | | | SIGNATURE |
| 1443 | PR00886C | 11.84 | 3.250e−23 | 110-128 | PR00886 | HIGH MOBILITY GROUP |
| | | | | | | (HMG1/HMG2) PROTEIN |
| | | | | | | SIGNATURE |
| 1443 | PR00886D | 15.72 | 6.786e−24 | 128-147 | PR00886 | HIGH MOBILITY GROUP |
| | | | | | | (HMG1/HMG2) PROTEIN |
| | | | | | | SIGNATURE |
| 1443 | PR00886E | 8.85 | 5.101e−09 | 63-83 | PR00886 | HIGH MOBILITY GROUP |
| | | | | | | (HMG1/HMG2) PROTEIN |
| | | | | | | SIGNATURE |
| 1443 | PR00886E | 8.85 | 9.182e−26 | 147-167 | PR00886 | HIGH MOBILITY GROUP |
| | | | | | | (HMG1/HMG2) PROTEIN |
| | | | | | | SIGNATURE |
| 1444 | PR00690A | 10.86 | 9.866e−09 | 782-800 | PR00690 | ADHESIN FAMILY |
| | | | | | | SIGNATURE |
| 1446 | BL00048 | 6.39 | 6.625e−09 | 11-37 | BL00048 | Protamine P1 proteins. |
| 1449 | DM00179 | 13.97 | 7.158e−10 | 107-116 | DM00179 | w KINASE ALPHA ADHESION |
| | | | | | | T-CELL. |
| 1453 | BL00056A | 28.90 | 3.769e−32 | 75-114 | BL00056 | Ribosomal protein S17 |
| | | | | | | proteins. |
| 1453 | BL00056B | 20.86 | 6.727e−23 | 123-146 | BL00056 | Ribosomal protein S17 |
| | | | | | | proteins. |
| 1453 | PR00973A | 20.76 | 2.565e−20 | 89-112 | PR00973 | RIBOSOMAL PROTEIN S17 |
| | | | | | | FAMILY SIGNATURE |
| 1453 | PR00973B | 13.27 | 6.625e−12 | 123-133 | PR00973 | RIBOSOMAL PROTEIN S17 |
| | | | | | | FAMILY SIGNATURE |
| 1453 | PR00973C | 10.86 | 1.300e−09 | 133-140 | PR00973 | RIBOSOMAL PROTEIN S17 |
| | | | | | | FAMILY SIGNATURE |
| 1454 | BL01019A | 13.20 | 9.520e−13 | 46-85 | BL01019 | ADP-ribosylation |
| | | | | | | factors family |
| | | | | | | proteins. |
| 1454 | BL0115A | 10.22 | 5.075e−11 | 12-55 | BL01115 | GTP-binding nuclear |
| | | | | | | protein ran proteins. |
| 1454 | PR00449A | 13.20 | 5.846e−21 | 12-33 | PR00449 | TRANSFORMING PROTEIN |
| | | | | | | P21 RAS SIGNATURE |
| 1454 | PR00449B | 14.34 | 5.500e−13 | 35-51 | PR00449 | TRANSFORMING PROTEIN |
| | | | | | | P21 RAS SIGNATURE |
| 1454 | PR00449C | 17.27 | 3.700e−24 | 53-75 | PR00449 | TRANSFORMING PROTEIN |
| | | | | | | P21 RAS SIGNATURE |
| 1454 | PR00449D | 10.79 | 3.368e−13 | 115-128 | PR00449 | TRANSFORMING PROTEIN |
| | | | | | | P21 RAS SIGNATURE |
| 1454 | PR00449E | 13.50 | 6.684e−17 | 150-172 | PR00449 | TRANSFORMING PROTEIN |
| | | | | | | P21 RAS SIGNATURE |
| 1458 | BL00284A | 15.64 | 2.742e−15 | 166-189 | BL00284 | Serpins proteins. |
| 1458 | BL00284B | 17.99 | 6.182e−12 | 258-278 | BL00284 | Serpins proteins. |
| 1458 | BL00284C | 28.56 | 6.400e−25 | 285-326 | BL00284 | Serpins proteins. |
| 1458 | BL00284D | 16.34 | 7.070e−12 | 396-422 | BL00284 | Serpins proteins. |
| 1458 | BL00284E | 19.15 | 7.968e−16 | 474-498 | BL00284 | Serpins proteins. |
| 1458 | PD02080A | 10.03 | 9.750e−10 | 1-16 | PD02080 | T-CELL GLYCOPROTEIN CD8 |
| | | | | | | CHAIN SURFACE ALPHA |
| | | | | | | PRE. |
| 1458 | PR00743B | 14.95 | 8.831e−09 | 206-226 | PR00743 | GLYCOSYL HYDROLASE |
| | | | | | | FAMILY 36 SIGNATURE |
| 1459 | BL00192A | 11.90 | 4.857e−30 | 5-44 | BL00192 | Cytochrome b/b6 heme- |
| | | | | | | ligand proteins. |
| 1459 | BL00192B | 9.13 | 1.000e−40 | 62-107 | BL00192 | Cytochrome b/b6 heme- |
| | | | | | | ligand proteins. |
| 1459 | BL00192C | 7.84 | 9.325e−40 | 124-171 | BL00192 | Cytochrome b/b6 heme- |
| | | | | | | ligand proteins. |
| 1459 | BL00192D | 10.29 | 4.176e−36 | 196-231 | BL00192 | Cytochrome b/b6 heme- |
| | | | | | | ligand proteins. |
| 1459 | BL00192E | 13.29 | 1.000e−40 | 247-294 | BL00192 | Cytochrome b/b6 heme- |
| | | | | | | ligand proteins. |
| 1459 | BL00192F | 14.83 | 1.900e−32 | 318-367 | BL00192 | Cytochrome b/b6 heme- |
| | | | | | | ligand proteins. |
| 1460 | BL00580A | 17.63 | 7.652e−37 | 14-49 | BL00580 | Ribosomal protein L32e |
| | | | | | | proteins. |
| 1460 | BL00580B | 19.83 | 6.850e−40 | 50-86 | BL00580 | Ribosomal protein L32e |
| | | | | | | proteins. |
| 1462 | BL00166A | 14.82 | 7.429e−10 | 66-77 | BL00166 | Enoyl-CoA |
| | | | | | | hydratase/isomerase |
| | | | | | | proteins. |
| 1462 | BL00166B | 16.92 | 3.348e−17 | 104-125 | BL00166 | Enoyl-CoA |
| | | | | | | hydratase/isomerase |
| | | | | | | proteins. |
| 1462 | BL00166C | 18.93 | 9.129e−23 | 161-187 | BL00166 | Enoyl-CoA |
| | | | | | | hydratase/isomerase |
| | | | | | | proteins. |
| 1462 | BL00166D | 22.87 | 4.375e−22 | 213-248 | BL00166 | Enoyl-CoA |
| | | | | | | hydratase/isomerase |
| | | | | | | proteins. |
| 1463 | BL00226A | 12.77 | 2.080e−14 | 83-97 | BL00226 | Intermediate filaments |
| | | | | | | proteins. |
| 1463 | BL00226B | 23.86 | 5.050e−38 | 183-230 | BL00226 | Intermediate filaments |
| | | | | | | proteins. |
| 1463 | BL00226C | 13.23 | 6.850e−25 | 248-278 | BL00226 | Intermediate filaments |
| | | | | | | proteins. |
| 1463 | BL00226D | 19.10 | 1.000e−40 | 349-395 | BL00226 | Intermediate filaments |
| | | | | | | proteins. |
| 1463 | BL00226D | 19.10 | 9.100e−09 | 296-342 | BL00226 | Intermediate filaments |
| | | | | | | proteins. |
| 1464 | BL00039A | 18.44 | 1.871e−23 | 21-59 | BL00039 | DEAD-box subfamily ATP- |
| | | | | | | dependent helicases |
| | | | | | | proteins. |
| 1464 | BL00039B | 19.19 | 4.064e−11 | 277-302 | BL00039 | DEAD-box subfamily ATP- |
| | | | | | | dependent helicases |
| | | | | | | proteins. |
| 1464 | BL00039C | 15.63 | 1.720e−11 | 364-387 | BL00039 | DEAD-box subfamily ATP- |
| | | | | | | dependent helicases |
| | | | | | | proteins. |
| 1464 | BL00039D | 21.67 | 1.900e−29 | 568-613 | BL00039 | DEAD-box subfamily ATP- |
| | | | | | | dependent helicases |
| | | | | | | proteins. |
| 1465 | BL00222B | 11.09 | 4.420e−10 | 60-75 | BL00222 | Insulin-like growth |
| | | | | | | factor binding |
| | | | | | | proteins. |
| 1465 | BL00273 | 12.24 | 8.286e−09 | 64-76 | BL00273 | Heat-stable |
| | | | | | | enterotoxins proteins. |
| 1465 | BL00282 | 16.88 | 4.234e−12 | 111-133 | BL00282 | Kazal serine protease |
| | | | | | | inhibitors family |
| | | | | | | proteins. |
| 1465 | PR00290B | 9.78 | 4.326e−09 | 122-133 | PR00290 | KAZAL-TYPE SERINE |
| | | | | | | PROTEASE INHIBITOR |
| | | | | | | SIGNATURE |
| 1465 | PR00834A | 9.80 | 3.659e−11 | 213-225 | PR00834 | HTRA/DEGQ PROTEASE |
| | | | | | | FAMILY SIGNATURE |
| 1465 | PR00834B | 10.09 | 5.500e−14 | 234-254 | PR00834 | HTRA/DEGQ PROTEASE |
| | | | | | | FAMILY SIGNATURE |
| 1465 | PR00834C | 15.43 | 3.613e−20 | 275-299 | PR00834 | HTRA/DEGQ PROTEASE |
| | | | | | | FAMILY SIGNATURE |
| 1465 | PR00834D | 12.14 | 6.455e−18 | 313-330 | PR00834 | HTRA/DEGQ PROTEASE |
| | | | | | | FAMILY SIGNATURE |
| 1465 | PR00834E | 13.63 | 5.355e−13 | 335-352 | PR00834 | HTRA/DEGQ PROTEASE |
| | | | | | | FAMILY SIGNATURE |
| 1465 | PR00834F | 10.91 | 9.526e−12 | 427-439 | PR00834 | HTRA/DEGQ PROTEASE |
| | | | | | | FAMILY SIGNATURE |
| 1466 | PR00939A | 8.95 | 2.636e−09 | 135-144 | PR00939 | C2HC-TYPE ZINC-FINGER |
| | | | | | | SIGNATURE |
| 1466 | PR00939A | 8.95 | 8.714e−11 | 96-105 | PR00939 | C2HC-TYPE ZINC-FINGER |
| | | | | | | SIGNATURE |
| 1466 | PR00939B | 13.27 | 2.588e−09 | 81-89 | PR00939 | C2HC-TYPE ZINC-FINGER |
| | | | | | | SIGNATURE |
| 1467 | BL00288A | 17.47 | 3.333e−34 | 17-46 | BL00288 | Tissue inhibitors of |
| | | | | | | metalloproteinases |
| | | | | | | proteins. |
| 1467 | BL00288B | 9.44 | 5.500e−14 | 62-72 | BL00288 | Tissue inhibitors of |
| | | | | | | metalloproteinases |
| | | | | | | proteins. |
| 1467 | BL00288C | 14.62 | 3.829e−16 | 78-92 | BL00288 | Tissue inhibitors of |
| | | | | | | metalloproteinases |
| | | | | | | proteins. |
| 1467 | BL00288D | 25.76 | 1.000e−40 | 100-144 | BL00288 | Tissue inhibitors of |
| | | | | | | metalloproteinases |
| | | | | | | proteins. |
| 1467 | BL00288E | 17.24 | 4.333e−26 | 145-167 | BL00288 | Tissue inhibitors of |
| | | | | | | metalloproteinases |
| | | | | | | proteins. |
| 1468 | PR00450C | 12.22 | 4.214e−11 | 98-119 | PR00450 | RECOVERIN FAMILY |
| | | | | | | SIGNATURE |
| 1469 | DM01282A | 17.37 | 7.179e−10 | 62-83 | DM01282 | w INTEGRASE CP4-57 SYT |
| | | | | | | P4. |
| 1470 | BL00359A | 20.66 | 4.000e−21 | 106-141 | BL00359 | Ribosomal protein L11 |
| | | | | | | proteins. |
| 1470 | BL00359B | 23.07 | 4.231e−24 | 142-182 | BL00359 | Ribosomal protein L11 |
| | | | | | | proteins. |
| 1470 | BL00359C | 22.18 | 6.586e−22 | 197-230 | BL00359 | Ribosomal protein L11 |
| | | | | | | proteins. |
| 1470 | BL01108A | 20.33 | 1.000e−08 | 126-158 | BL01108 | Ribosomal protein L24 |
| | | | | | | proteins. |
| 1472 | BL00390 | 27.44 | 1.000e−40 | 16-70 | BL00390 | Sodium and potassium |
| | | | | | | ATPases beta subunits |
| | | | | | | proteins. |
| 1473 | BL00536A | 14.17 | 1.000e−40 | 449-490 | BL00536 | Ubiquitin-activating |
| | | | | | | enzyme proteins. |
| 1473 | BL00536A | 14.17 | 9.647e−11 | 53-94 | BL00536 | Ubiquitin-activating |
| | | | | | | enzyme proteins. |
| 1473 | BL00536B | 18.17 | 1.000e−40 | 498-541 | BL00536 | Ubiquitin-activating |
| | | | | | | enzyme proteins. |
| 1473 | BL00536B | 18.17 | 3.947e−09 | 97-140 | BL00536 | Ubiquitin-activating |
| | | | | | | enzyme proteins. |
| 1473 | BL00536C | 15.91 | 1.000e−40 | 559-597 | BL00536 | Ubiquitin-activating |
| | | | | | | enzyme proteins. |
| 1473 | BL00536D | 22.91 | 1.000e−40 | 621-664 | BL00536 | Ubiquitin-activating |
| | | | | | | enzyme proteins. |
| 1473 | BL00536E | 16.94 | 3.753e−09 | 744-774 | BL00536 | Ubiquitin-activating |
| | | | | | | enzyme proteins. |
| 1473 | BL00536E | 16.94 | 8.435e−34 | 841-871 | BL00536 | Ubiquitin-activating |
| | | | | | | enzyme proteins. |
| 1473 | BL00536F | 13.65 | 1.000e−40 | 872-910 | BL00536 | Ubiquitin-activating |
| | | | | | | enzyme proteins. |
| 1476 | BL00227A | 24.55 | 3.250e−39 | 1-34 | BL00227 | Tubulin subunits alpha, |
| | | | | | | beta, and gamma |
| | | | | | | proteins. |
| 1476 | BL00227B | 19.29 | 1.000e−40 | 50-104 | BL00227 | Tubulin subunits alpha, |
| | | | | | | beta, and gamma |
| | | | | | | proteins. |
| 1476 | BL00227C | 25.48 | 1.000e−40 | 111-162 | BL00227 | Tubulin subunits alpha, |
| | | | | | | beta, and gamma |
| | | | | | | proteins. |
| 1476 | BL00227D | 18.46 | 1.000e−40 | 220-273 | BL00227 | Tubulin subunits alpha, |
| | | | | | | beta, and gamma |
| | | | | | | proteins. |
| 1476 | BL00227E | 24.15 | 8.500e−34 | 324-358 | BL00227 | Tubulin subunits alpha, |
| | | | | | | beta, and gamma |
| | | | | | | proteins. |
| 1476 | BL00227F | 21.16 | 1.000e−40 | 372-425 | BL00227 | Tubulin subunits alpha, |
| | | | | | | beta, and gamma |
| | | | | | | proteins. |
| 1477 | PD01443B | 15.02 | 1.000e−40 | 20-70 | PD01443 | INHIBITOR CALPAIN |
| | | | | | | CALPASTATIN REPEAT |
| | | | | | | THIOL PROT. |
| 1477 | PD01443C | 9.45 | 3.455e−40 | 78-112 | PD01443 | INHIBITOR CALPAIN |
| | | | | | | CALPASTATIN REPEAT |
| | | | | | | THIOL PROT. |
| 1477 | PD01443D | 8.36 | 8.031e−26 | 113-134 | PD01443 | INHIBITOR CALPAIN |
| | | | | | | CALPASTATIN REPEAT |
| | | | | | | THIOL PROT. |
| 1477 | PD01443E | 9.22 | 1.273e−34 | 136-162 | PD01443 | INHIBITOR CALPAIN |
| | | | | | | CALPASTATIN REPEAT |
| | | | | | | THIOL PROT. |
| 1477 | PD01443F | 13.86 | 1.000e−40 | 163-213 | PD01443 | INHIBITOR CALPAIN |
| | | | | | | CALPASTATIN REPEAT |
| | | | | | | THIOL PROT. |
| 1477 | PD01443G | 11.15 | 1.000e−40 | 228-272 | PD01443 | INHIBITOR CALPAIN |
| | | | | | | CALPASTATIN REPEAT |
| | | | | | | THIOL PROT. |
| 1477 | PD01443H | 11.91 | 3.032e−31 | 273-298 | PD01443 | INHIBITOR CALPAIN |
| | | | | | | CALPASTATIN REPEAT |
| | | | | | | THIOL PROT. |
| 1477 | PD01443H | 11.91 | 6.032e−11 | 552-577 | PD01443 | INHIBITOR CALPAIN |
| | | | | | | CALPASTATIN REPEAT |
| | | | | | | THIOL PROT. |
| 1477 | PD01443H | 11.91 | 8.835e−13 | 415-440 | PD01443 | INHIBITOR CALPAIN |
| | | | | | | CALPASTATIN REPEAT |
| | | | | | | THIOL PROT. |
| 1477 | PD01443I | 10.25 | 1.000e−40 | 299-335 | PD01443 | INHIBITOR CALPAIN |
| | | | | | | CALPASTATIN REPEAT |
| | | | | | | THIOL PROT. |
| 1477 | PD01443J | 4.11 | 1.545e−15 | 523-556 | PD01443 | INHIBITOR CALPAIN |
| | | | | | | CALPASTATIN REPEAT |
| | | | | | | THIOL PROT. |
| 1477 | PD01443J | 4.11 | 2.756e−40 | 386-419 | PD01443 | INHIBITOR CALPAIN |
| | | | | | | CALPASTATIN REPEAT |
| | | | | | | THIOL PROT. |
| 1477 | PD01443J | 4.11 | 5.425e−10 | 243-276 | PD01443 | INHIBITOR CALPAIN |
| | | | | | | CALPASTATIN REPEAT |
| | | | | | | THIOL PROT. |
| 1477 | PD01443K | 9.19 | 1.000e−40 | 420-458 | PD01443 | INHIBITOR CALPAIN |
| | | | | | | CALPASTATIN REPEAT |
| | | | | | | THIOL PROT. |
| 1477 | PD01443K | 9.19 | 3.571e−23 | 278-316 | PD01443 | INHIBITOR CALPAIN |
| | | | | | | CALPASTATIN REPEAT |
| | | | | | | THIOL PROT. |
| 1477 | PD01443K | 9.19 | 9.914e−15 | 557-595 | PD01443 | INHIBITOR CALPAIN |
| | | | | | | CALPASTATIN REPEAT |
| | | | | | | THIOL PROT. |
| 1477 | PD01443L | 14.60 | 1.000e−40 | 459-501 | PD01443 | INHIBITOR CALPAIN |
| | | | | | | CALPASTATIN REPEAT |
| | | | | | | THIOL PROT. |
| 1477 | PD01443M | 11.28 | 1.000e−40 | 521-554 | PD01443 | INHIBITOR CALPAIN |
| | | | | | | CALPASTATIN REPEAT |
| | | | | | | THIOL PROT. |
| 1477 | PD01443M | 11.28 | 7.387e−09 | 241-274 | PD01443 | INHIBITOR CALPAIN |
| | | | | | | CALPASTATIN REPEAT |
| | | | | | | THIOL PROT. |
| 1477 | PD01443M | 11.28 | 7.861e−14 | 384-417 | PD01443 | INHIBITOR CALPAIN |
| | | | | | | CALPASTATIN REPEAT |
| | | | | | | THIOL PROT. |
| 1477 | PD01443N | 11.76 | 1000e−40 | 555-592 | PD01443 | INHIBITOR CALPAIN |
| | | | | | | CALPASTATIN REPEAT |
| | | | | | | THIOL PROT. |
| 1477 | PD01443N | 11.76 | 4.744e−09 | 142-179 | PD01443 | INHIBITOR CALPAIN |
| | | | | | | CALPASTATIN REPEAT |
| | | | | | | THIOL PROT. |
| 1477 | PD01443N | 11.76 | 7.341e−19 | 418-455 | PD01443 | INHIBITOR CALPAIN |
| | | | | | | CALPASTATIN REPEAT |
| | | | | | | THIOL PROT. |
| 1477 | PD01443N | 11.76 | 9.563e−14 | 276-313 | PD01443 | INHIBITOR CALPAIN |
| | | | | | | CALPASTATIN REPEAT |
| | | | | | | THIOL PROT. |
| 1477 | PD01443O | 6.25 | 4.600e−33 | 640-667 | PD01443 | INHIBITOR CALPAIN |
| | | | | | | CALPASTATIN REPEAT |
| | | | | | | THIOL PROT. |
| 1479 | PR00318C | 12.09 | 8.254e−09 | 268-285 | PR00318 | ALPHA G-PROTEIN |
| | | | | | | (TRANSDUCIN) SIGNATURE |
| 1479 | PR00440C | 9.54 | 7.000e−09 | 268-285 | PR00440 | G-PROTEIN ALPHA SUBUNIT |
| | | | | | | GROUP 12 SIGNATURE |
| 1480 | BL00719A | 16.66 | 6.885e−12 | 32-45 | BL00719 | Glycosyl hydrolases |
| | | | | | | family 2 proteins. |
| 1480 | BL00719B | 17.24 | 5.333e−22 | 207-231 | BL00719 | Glycosyl hydrolases |
| | | | | | | family 2 proteins. |
| 1480 | BL00719C | 30.44 | 3.000e−37 | 324-368 | BL00719 | Glycosyl hydrolases |
| | | | | | | family 2 proteins. |
| 1480 | BL00719D | 22.90 | 4.103e−37 | 369-406 | BL00719 | Glycosyl hydrolases |
| | | | | | | family 2 proteins. |
| 1480 | BL00719E | 16.22 | 2.909e−18 | 436-451 | BL00719 | Glycosyl hydrolases |
| | | | | | | family 2 proteins. |
| 1480 | BL00719F | 17.71 | 5.765e−14 | 533-548 | BL00719 | Glycosyl hydrolases |
| | | | | | | family 2 proteins. |
| 1480 | BL00719G | 25.30 | 4.000e−23 | 588-617 | BL00719 | Glycosyl hydrolases |
| | | | | | | family 2 proteins. |
| 1480 | PR00132A | 13.45 | 8.541e−13 | 126-141 | PR00132 | GLYCOSYL HYDROLASE |
| | | | | | | FAMILY 2 SIGNATURE |
| 1480 | PR00132B | 10.72 | 2.688e−18 | 338-352 | PR00132 | GLYCOSYL HYDROLASE |
| | | | | | | FAMILY 2 SIGNATURE |
| 1480 | PR00132C | 18.68 | 5.500e−20 | 369-387 | PR00132 | GLYCOSYL HYDROLASE |
| | | | | | | FAMILY 2 SIGNATURE |
| 1480 | PR00132D | 16.76 | 9.036e−19 | 437-452 | PR00132 | GLYCOSYL HYDROLASE |
| | | | | | | FAMILY 2 SIGNATURE |
| 1480 | PR00132E | 20.16 | 8.054e−14 | 534-549 | PR00132 | GLYCOSYL HYDROLASE |
| | | | | | | FAMILY 2 SIGNATURE |
| 1481 | BL00678 | 9.67 | 1.947e−09 | 423-433 | BL00678 | Trp-Asp (WD) repeat |
| | | | | | | proteins proteins. |
| 1481 | PF00646A | 14.37 | 8.594e−09 | 237-250 | PF00646 | F-box domain proteins. |
| 1481 | PR00320A | 16.74 | 2.059e−10 | 421-435 | PR00320 | G-PROTEIN BETA WD-40 |
| | | | | | | REPEAT SIGNATURE |
| 1481 | PR00320A | 16.74 | 3.382e−10 | 513-527 | PR00320 | G-PROTEIN BETA WD-40 |
| | | | | | | REPEAT SIGNATURE |
| 1481 | PR00320B | 12.19 | 4.857e−10 | 421-435 | PR00320 | G-PROTEIN BETA WD-40 |
| | | | | | | REPEAT SIGNATURE |
| 1481 | PR00320C | 13.01 | 7.480e−10 | 421-435 | PR00320 | G-PROTEIN BETA WD-40 |
| | | | | | | REPEAT SIGNATURE |
| 1481 | PR00320C | 13.01 | 7.900e−09 | 513-527 | PR00320 | G-PROTEIN BETA WD-40 |
| | | | | | | REPEAT SIGNATURE |
| 1483 | BL01115A | 10.22 | 1.000e−40 | 11-54 | BL01115 | GTP-binding nuclear |
| | | | | | | protein ran proteins. |
| 1483 | BL01115B | 10.81 | 1.000e−40 | 91-134 | BL01115 | GTP-binding nuclear |
| | | | | | | protein ran proteins. |
| 1483 | BL01115C | 10.52 | 2.200e−39 | 140-170 | BL01115 | GTP-binding nuclear |
| | | | | | | protein ran proteins. |
| 1483 | BL01115D | 14.49 | 1.360e−28 | 184-215 | BL01115 | GTP-binding nuclear |
| | | | | | | protein ran proteins. |
| 1483 | PR00442A | 6.46 | 8.013e−09 | 12-27 | PR00442 | G-PROTEIN ALPHA SUBUNIT |
| | | | | | | GROUP Q SIGNATURE |
| 1483 | PR00449A | 13.20 | 1.000e−16 | 11-32 | PR00449 | TRANSFORMING PROTEIN |
| | | | | | | P21 RAS SIGNATURE |
| 1483 | PR00449B | 14.34 | 5.320e−10 | 34-50 | PR00449 | TRANSFORMING PROTEIN |
| | | | | | | P21 RAS SIGNATURE |
| 1483 | PR00449C | 17.27 | 4.750e−16 | 52-74 | PR00449 | TRANSFORMING PROTEIN |
| | | | | | | P21 RAS SIGNATURE |
| 1483 | PR00449D | 10.79 | 9.053e−13 | 113-126 | PR00449 | TRANSFORMING PROTEIN |
| | | | | | | P21 RAS SIGNATURE |
| 1483 | PR00449E | 13.50 | 1.391e−15 | 146-168 | PR00449 | TRANSFORMING PROTEIN |
| | | | | | | P21 RAS SIGNATURE |
| 1483 | PR00627A | 10.81 | 1.265e−19 | 24-38 | PR00627 | GTP-BINDING NUCLEAR |
| | | | | | | PROTEIN RAN/TC4 FAMILY |
| | | | | | | SIGNATURE |
| 1483 | PR00627B | 11.05 | 6.538e−25 | 71-89 | PR00627 | GTP-BINDING NUCLEAR |
| | | | | | | PROTEIN RAN/TC4 FAMILY |
| | | | | | | SIGNATURE |
| 1483 | PR00627C | 11.68 | 9.400e−29 | 91-112 | PR00627 | GTP-BINDING NUCLEAR |
| | | | | | | PROTEIN RAN/TC4 FAMILY |
| | | | | | | SIGNATURE |
| 1483 | PR00627D | 12.54 | 1.000e−21 | 127-145 | PR00627 | GTP-BINDING NUCLEAR |
| | | | | | | PROTEIN RAN/TC4 FAMILY |
| | | | | | | SIGNATURE |
| 1483 | PR00627E | 14.59 | 1.529e−24 | 165-187 | PR00627 | GTP-BINDING NUCLEAR |
| | | | | | | PROTEIN RAN/TC4 FAMILY |
| | | | | | | SIGNATURE |
| 1484 | BL00290A | 20.89 | 6.143e−17 | 140-162 | BL00290 | Immunoglobulins and |
| | | | | | | major |
| | | | | | | histocompatibility |
| | | | | | | complex proteins. |
| 1484 | BL00290B | 13.17 | 4.750e−16 | 197-214 | BL00290 | Immunoglobulins and |
| | | | | | | major |
| | | | | | | histocompatibility |
| | | | | | | complex proteins. |
| 1484 | PF00969A | 22.07 | 9.719e−33 | 11-53 | PF00969 | Class II |
| | | | | | | histocompatibility |
| | | | | | | antigen, beta domain |
| | | | | | | proteins. |
| 1484 | PF00969B | 9.97 | 1.000e−40 | 55-90 | PF00969 | Class II |
| | | | | | | histocompatibility |
| | | | | | | antigen, beta domain |
| | | | | | | proteins. |
| 1484 | PF00969C | 27.72 | 1.000e−40 | 94-143 | PF00969 | Class II |
| | | | | | | histocompatibility |
| | | | | | | antigen, beta domain |
| | | | | | | proteins. |
| 1484 | PF00969D | 14.02 | 7.375e−35 | 151-180 | PF00969 | Class II |
| | | | | | | histocompatibility |
| | | | | | | antigen, beta domain |
| | | | | | | proteins. |
| 1484 | PF00969E | 11.49 | 7.686e−35 | 209-243 | PF00969 | Class II |
| | | | | | | histocompatibility |
| | | | | | | antigen, beta domain |
| | | | | | | proteins. |
| 1486 | BL00309C | 18.65 | 2.241e−09 | 62-86 | BL00309 | Vertebrate galactoside- |
| | | | | | | binding lectin |
| | | | | | | proteins. |
| 1487 | BL00824B | 9.21 | 3.676e−09 | 349-368 | BL00824 | Elongation factor 1 |
| | | | | | | beta/beta′/delta chain |
| | | | | | | proteins. |
| 1487 | PF00956A | 11.88 | 1.000e−13 | 121-131 | PF00956 | Nuclesosome assembly |
| | | | | | | protein (NAP). |
| 1487 | PF00956B | 23.14 | 1.000e−40 | 162-202 | PF00956 | Nuclesosome assembly |
| | | | | | | protein (NAP). |
| 1487 | PF00956C | 7.72 | 6.850e−22 | 216-233 | PF00956 | Nuclesosome assembly |
| | | | | | | protein (NAP). |
| 1487 | PF00956D | 7.51 | 3.700e−12 | 295-305 | PF00956 | Nuclesosome assembly |
| | | | | | | protein (NAP). |
| 1488 | BL00284A | 15.64 | 1.000e−18 | 50-73 | BL00284 | Serpins proteins. |
| 1488 | BL00284B | 17.99 | 1.273e−12 | 149-169 | BL00284 | Serpins proteins. |
| 1488 | BL00284C | 28.56 | 5.821e−27 | 179-220 | BL00284 | Serpins proteins. |
| 1488 | BL00284D | 16.34 | 1.500e−14 | 291-317 | BL00284 | Serpins proteins. |
| 1488 | BL00284E | 19.15 | 3.842e−21 | 374-398 | BL00284 | Serpins proteins. |
| 1489 | BL00420B | 22.67 | 3.951e−34 | 28-82 | BL00420 | Speract receptor repeat |
| | | | | | | proteins domain |
| | | | | | | proteins. |
| 1489 | BL00420C | 11.90 | 3.864e−10 | 113-123 | BL00420 | Speract receptor repeat |
| | | | | | | proteins domain |
| | | | | | | proteins. |
| 1489 | PR00258A | 11.46 | 8.054e−16 | 24-40 | PR00258 | SPERACT RECEPTOR |
| | | | | | | SIGNATURE |
| 1489 | PR00258B | 9.63 | 3.531e−15 | 43-54 | PR00258 | SPERACT RECEPTOR |
| | | | | | | SIGNATURE |
| 1489 | PR00258C | 9.05 | 4.643e−11 | 58-68 | PR00258 | SPERACT RECEPTOR |
| | | | | | | SIGNATURE |
| 1489 | PR00258D | 14.41 | 7.968e−15 | 89-103 | PR00258 | SPERACT RECEPTOR |
| | | | | | | SIGNATURE |
| 1489 | PR00258E | 13.33 | 1.486e−13 | 112-124 | PR00258 | SPERACT RECEPTOR |
| | | | | | | SIGNATURE |
| 1490 | BL00474A | 9.96 | 2.800e−15 | 46-56 | BL00474 | Ribosomal protein L3 |
| | | | | | | proteins. |
| 1490 | BL00474B | 21.72 | 7.500e−24 | 80-109 | BL00474 | Ribosomal protein L3 |
| | | | | | | proteins. |
| 1490 | BL00474C | 21.74 | 6.571e−34 | 211-245 | BL00474 | Ribosomal protein L3 |
| | | | | | | proteins. |
| 1490 | BL00474D | 29.47 | 8.650e−36 | 252-288 | BL00474 | Ribosomal protein L3 |
| | | | | | | proteins. |
| 1491 | BL00227A | 24.55 | 8.200e−36 | 1-34 | BL00227 | Tubulin subunits alpha, |
| | | | | | | beta, and gamma |
| | | | | | | proteins. |
| 1491 | BL00227B | 19.29 | 1.000e−40 | 52-106 | BL00227 | Tubulin subunits alpha, |
| | | | | | | beta, and gamma |
| | | | | | | proteins. |
| 1491 | BL00227C | 25.48 | 1.000e−40 | 113-164 | BL00227 | Tubulin subunits alpha, |
| | | | | | | beta, and gamma |
| | | | | | | proteins. |
| 1491 | BL00227D | 18.46 | 1.000e−40 | 222-275 | BL00227 | Tubulin subunits alpha, |
| | | | | | | beta, and gamma |
| | | | | | | proteins. |
| 1491 | BL00227E | 24.15 | 5.000e−37 | 326-360 | BL00227 | Tubulin subunits alpha, |
| | | | | | | beta, and gamma |
| | | | | | | proteins. |
| 1491 | BL00227F | 21.16 | 1.000e−40 | 382-435 | BL00227 | Tubulin subunits alpha, |
| | | | | | | beta, and gamma |
| | | | | | | proteins. |
| 1493 | BL00018 | 7.41 | 6.870e−09 | 172-184 | BL00018 | EF-hand calcium-binding |
| | | | | | | domain proteins. |
| 1493 | PR00049D | 0.00 | 9.214e−10 | 63-77 | PR00049 | WILM'S TUMOUR PROTEIN |
| | | | | | | SIGNATURE |
| 1494 | DM01111A | 10.80 | 5.500e−34 | 7-35 | DM01111 | 4 kw PHOSPHATASE |
| | | | | | | TRANSFORMING 61K PDF1. |
| 1494 | DM01111B | 9.44 | 1.000e−40 | 36-72 | DM01111 | 4 kw PHOSPHATASE |
| | | | | | | TRANSFORMING 61K PDF1. |
| 1494 | DM01111C | 9.35 | 9.100e−35 | 93-121 | DM01111 | 4 kw PHOSPHATASE |
| | | | | | | TRANSFORMING 61K PDF1. |
| 1494 | DM01111D | 16.76 | 1.000e−40 | 152-197 | DM01111 | 4 kw PHOSPHATASE |
| | | | | | | TRANSFORMING 61K PDF1. |
| 1494 | DM01111E | 17.28 | 1.000e−40 | 238-286 | DM01111 | 4 kw PHOSPHATASE |
| | | | | | | TRANSFORMING 61K PDF1. |
| 1494 | DM01111E | 17.28 | 7.632e−10 | 398-446 | DM01111 | 4 kw PHOSPHATASE |
| | | | | | | TRANSFORMING 61K PDF1. |
| 1494 | DM01111F | 18.22 | 6.684e−40 | 305-340 | DM01111 | 4 kw PHOSPHATASE |
| | | | | | | TRANSFORMING 61K PDF1. |
| 1494 | DM01111G | 10.39 | 1.000e−40 | 341-381 | DM01111 | 4 kw PHOSPHATASE |
| | | | | | | TRANSFORMING 61K PDF1. |
| 1494 | DM01111G | 10.39 | 5.376e−11 | 181-221 | DM01111 | 4 kw PHOSPHATASE |
| | | | | | | TRANSFORMING 61K PDF1. |
| 1494 | DM01111G | 10.39 | 9.622e−09 | 259-299 | DM01111 | 4 kw PHOSPHATASE |
| | | | | | | TRANSFORMING 61K PDF1. |
| 1494 | DM01111H | 12.57 | 1.000e−40 | 382-423 | DM01111 | 4 kw PHOSPHATASE |
| | | | | | | TRANSFORMING 61K PDF1. |
| 1494 | DM01111I | 15.32 | 5.846e−36 | 424-453 | DM01111 | 4 kw PHOSPHATASE |
| | | | | | | TRANSFORMING 61K PDF1. |
| 1494 | DM01111J | 19.19 | 8.941e−40 | 454-487 | DM01111 | 4 kw PHOSPHATASE |
| | | | | | | TRANSFORMING 61K PDF1. |
| 1494 | DM01111K | 19.10 | 4.000e−38 | 494-523 | DM01111 | 4 kw PHOSPHATASE |
| | | | | | | TRANSFORMING 61K PDF1. |
| 1494 | DM01111L | 11.93 | 3.500e−33 | 524-551 | DM01111 | 4 kw PHOSPHATASE |
| | | | | | | TRANSFORMING 61K PDF1. |
| 1494 | DM01111M | 10.67 | 8.500e−30 | 560-583 | DM01111 | 4 kw PHOSPHATASE |
| | | | | | | TRANSFORMING 61K PDF1. |
| 1495 | BL01310 | 14.74 | 2.432e−29 | 133-168 | BL01310 | ATP1G1/PLM/MAT8 |
| | | | | | | family proteins. |
| 1496 | BL01221A | 17.26 | 1.000e−22 | 1-28 | BL01221 | PMP-22/EMP/MP20 |
| | | | | | | family proteins. |
| 1496 | BL01221B | 13.29 | 4.818e−12 | 34-47 | BL01221 | PMP-22/EMP/MP20 |
| | | | | | | family proteins. |
| 1496 | BL01221C | 26.20 | 2.303e−12 | 56-100 | BL01221 | PMP-22/EMP/MP20 |
| | | | | | | family proteins. |
| 1496 | BL01221C | 26.20 | 5.500e−33 | 59-103 | BL01221 | PMP-22/EMP/MP20 |
| | | | | | | family proteins. |
| 1496 | BL01221D | 13.99 | 6.276e−27 | 139-165 | BL01221 | PMP-22/EMP/MP20 |
| | | | | | | family proteins. |
| 1497 | BL00094A | 13.03 | 6.192e−16 | 1139-1159 | BL00094 | C-5 cytosine-specific |
| | | | | | | DNA methylases |
| | | | | | | proteins. |
| 1497 | BL00094B | 16.39 | 1.000e−14 | 1218-1233 | BL00094 | C-5 cytosine-specific |
| | | | | | | DNA methylases |
| | | | | | | proteins. |
| 1497 | BL00094C | 9.14 | 2.800e−11 | 1260-1271 | BL00094 | C-5 cytosine-specific |
| | | | | | | DNA methylases |
| | | | | | | proteins. |
| 1497 | BL00094D | 14.24 | 8.636e−14 | 1298-1317 | BL00094 | C-5 cytosine-specific |
| | | | | | | DNA methylases |
| | | | | | | proteins. |
| 1497 | BL00094E | 14.45 | 8.920e−15 | 1546-1561 | BL00094 | C-5 cytosine-specific |
| | | | | | | DNA methylases |
| | | | | | | proteins. |
| 1497 | BL00094F | 13.12 | 9.280e−10 | 1573-1582 | BL00094 | C-5 cytosine-specific |
| | | | | | | DNA methylases |
| | | | | | | proteins. |
| 1497 | PF01140D | 15.54 | 3.407e−09 | 212-246 | PF01140 | Matrix protein (MA), |
| | | | | | | p15. |
| 1497 | PR00105A | 10.36 | 4.900e−13 | 1140-1156 | PR00105 | CYTOSINE-SPECIFIC DNA |
| | | | | | | METHYLTRANSFERASE |
| | | | | | | SIGNATURE |
| 1497 | PR00105B | 12.32 | 2.800e−12 | 1259-1273 | PR00105 | CYTOSINE-SPECIFIC DNA |
| | | | | | | METHYLTRANSFERASE |
| | | | | | | SIGNATURE |
| 1497 | PR00105C | 10.86 | 1.000e−10 | 1305-1318 | PR001015 | CYTOSINE-SPECIFIC DNA |
| | | | | | | METHYLTRANSFERASE |
| | | | | | | SIGNATURE |
| 1498 | PR00801A | 10.74 | 4.913e−25 | 2-22 | PR00801 | ADRENOMEDULLIN |
| | | | | | | SIGNATURE |
| 1498 | PR00801B | 11.34 | 1.094e−22 | 30-46 | PR00801 | ADRENOMEDULLIN |
| | | | | | | SIGNATURE |
| 1498 | PR00801C | 10.94 | 8.258e−22 | 49-67 | PR00801 | ADRENOMEDULLIN |
| | | | | | | SIGNATURE |
| 1498 | PR00801D | 13.48 | 8.463e−22 | 83-100 | PR00801 | ADRENOMEDULLIN |
| | | | | | | SIGNATURE |
| 1498 | PR00801E | 9.31 | 5.829e−26 | 103-122 | PR00801 | ADRENOMEDULLIN |
| | | | | | | SIGNATURE |
| 1498 | PR00801F | 17.66 | 3.596e−25 | 122-141 | PR00801 | ADRENOMEDULLIN |
| | | | | | | SIGNATURE |
| 1498 | PR00801G | 10.72 | 1.000e−21 | 141-157 | PR00801 | ADRENOMEDULLIN |
| | | | | | | SIGNATURE |
| 1498 | PR00801H | 10.32 | 3.512e−19 | 161-178 | PR00801 | ADRENOMEDULLIN |
| | | | | | | SIGNATURE |
| 1499 | BL00303A | 21.77 | 5.000e−26 | 4-40 | BL00303 | S-100/ICaBP type |
| | | | | | | calcium binding |
| | | | | | | protein. |
| 1499 | BL00303B | 26.15 | 1.900e−29 | 48-84 | BL00303 | S-100/ICaBP type |
| | | | | | | calcium binding |
| | | | | | | protein. |
| 1503 | BL00027 | 26.43 | 4.000e−11 | 27-69 | BL00027 | ‘Homeobox’ domain |
| | | | | | | proteins. |
| 1504 | BL00913B | 10.94 | 7.706e−11 | 86-101 | BL00913 | Iron-containing alcohol |
| | | | | | | dehydrogenases |
| | | | | | | proteins. |
| 1504 | BL00913C | 7.62 | 4.375e−11 | 136-145 | BL00913 | Iron-containing alcohol |
| | | | | | | dehydrogenases |
| | | | | | | proteins. |
| 1504 | BL00913D | 24.20 | 8.981e−17 | 170-203 | BL00913 | Iron-containing alcohol |
| | | | | | | dehydrogenases |
| | | | | | | proteins. |
| 1504 | BL00913E | 21.38 | 9.203e−09 | 269-302 | BL00913 | Iron-containing alcohol |
| | | | | | | dehydrogenases |
| | | | | | | proteins. |
| 1506 | BL00298A | 10.97 | 1.000e−40 | 13-57 | BL00298 | Heat shock hsp90 |
| | | | | | | proteins family |
| | | | | | | proteins. |
| 1506 | BL00298B | 15.64 | 1.000e−40 | 73-119 | BL00298 | Heat shock hsp90 |
| | | | | | | proteins family |
| | | | | | | proteins. |
| 1506 | BL00298C | 16.40 | 1.000e−40 | 120-163 | BL00298 | Heat shock hsp90 |
| | | | | | | proteins family |
| | | | | | | proteins. |
| 1506 | BL00298D | 17.97 | 1.000e−40 | 176-215 | BL00298 | Heat shock hsp90 |
| | | | | | | proteins family |
| | | | | | | proteins. |
| 1506 | BL00298E | 27.30 | 1.000e−40 | 267-321 | BL00298 | Heat shock hsp90 |
| | | | | | | proteins family |
| | | | | | | proteins. |
| 1506 | BL00298F | 11.21 | 1.000e−40 | 353-407 | BL00298 | Heat shock hsp90 |
| | | | | | | proteins family |
| | | | | | | proteins. |
| 1506 | BL00298G | 24.57 | 1.000e−40 | 410-464 | BL00298 | Heat shock hsp90 |
| | | | | | | proteins family |
| | | | | | | proteins. |
| 1506 | BL00298H | 20.50 | 1.000e−40 | 498-551 | BL00298 | Heat shock hsp90 |
| | | | | | | proteins family |
| | | | | | | proteins. |
| 1506 | BL00298I | 30.07 | 1.000e−40 | 602-655 | BL00298 | Heat shock hsp90 |
| | | | | | | proteins family |
| | | | | | | proteins. |
| 1506 | PR00775A | 9.90 | 1.692e−24 | 13-33 | PR00775 | 90 KD HEAT SHOCK |
| | | | | | | PROTEIN SIGNATURE |
| 1506 | PR00775B | 3.52 | 2.286e−29 | 34-56 | PR00775 | 90 KD HEAT SHOCK |
| | | | | | | PROTEIN SIGNATURE |
| 1506 | PR00775C | 10.68 | 1.474e−19 | 83-100 | PR00775 | 90 KD HEAT SHOCK |
| | | | | | | PROTEIN SIGNATURE |
| 1506 | PR00775D | 8.91 | 1.000e−22 | 101-118 | PR00775 | 90 KD HEAT SHOCK |
| | | | | | | PROTEIN SIGNATURE |
| 1506 | PR00775E | 8.06 | 1.000e−27 | 126-148 | PR00775 | 90 KD HEAT SHOCK |
| | | | | | | PROTEIN SIGNATURE |
| 1506 | PR00775F | 12.76 | 7.188e−21 | 177-194 | PR00775 | 90 KD HEAT SHOCK |
| | | | | | | PROTEIN SIGNATURE |
| 1506 | PR00775G | 10.64 | 5.800e−22 | 195-213 | PR00775 | 90 KD HEAT SHOCK |
| | | | | | | PROTEIN SIGNATURE |
| 1507 | BL00223A | 15.59 | 7.158e−12 | 270-303 | BL00223 | Annexins repeat |
| | | | | | | proteins domain |
| | | | | | | proteins. |
| 1507 | BL00223A | 15.59 | 8.113e−16 | 38-71 | BL00223 | Annexins repeat |
| | | | | | | proteins domain |
| | | | | | | proteins. |
| 1507 | BL00223A | 15.59 | 9.625e−32 | 110-143 | BL00223 | Annexins repeat |
| | | | | | | proteins domain |
| | | | | | | proteins. |
| 1507 | BL00223B | 28.47 | 1.000e−40 | 180-229 | BL00223 | Annexins repeat |
| | | | | | | proteins domain |
| | | | | | | proteins. |
| 1507 | BL00223C | 24.79 | 1.000e−40 | 257-311 | BL00223 | Annexins repeat |
| | | | | | | proteins domain |
| | | | | | | proteins. |
| 1507 | BL00223C | 24.79 | 6.760e−18 | 97-151 | BL00223 | Annexins repeat |
| | | | | | | proteins domain |
| | | | | | | proteins. |
| 1507 | PR00196A | 11.16 | 3.423e−15 | 119-141 | PR00196 | ANNEXIN FAMILY |
| | | | | | | SIGNATURE |
| 1507 | PR00196A | 11.16 | 7.136e−23 | 47-69 | PR00196 | ANNEXIN FAMILY |
| | | | | | | SIGNATURE |
| 1507 | PR00196B | 10.68 | 7.300e−17 | 87-103 | PR00196 | ANNEXIN FAMILY |
| | | | | | | SIGNATURE |
| 1507 | PR00196C | 10.36 | 6.870e−23 | 114-135 | PR00196 | ANNEXIN FAMILY |
| | | | | | | SIGNATURE |
| 1507 | PR00196C | 10.36 | 7.750e−12 | 274-295 | PR00196 | ANNEXIN FAMILY |
| | | | | | | SIGNATURE |
| 1507 | PR00196D | 21.86 | 4.103e−25 | 198-224 | PR00196 | ANNEXIN FAMILY |
| | | | | | | SIGNATURE |
| 1507 | PR00196E | 9.19 | 1.333e−23 | 278-298 | PR00196 | ANNEXIN FAMILY |
| | | | | | | SIGNATURE |
| 1507 | PR00196E | 9.19 | 7.353e−12 | 118-138 | PR00196 | ANNEXIN FAMILY |
| | | | | | | SIGNATURE |
| 1507 | PR00196F | 13.89 | 2.714e−15 | 306-321 | PR00196 | ANNEXIN FAMILY |
| | | | | | | SIGNATURE |
| 1507 | PR00196G | 11.72 | 4.353e−09 | 162-175 | PR00196 | ANNEXIN FAMILY |
| | | | | | | SIGNATURE |
| 1507 | PR00196G | 11.72 | 6.000e−16 | 322-335 | PR00196 | ANNEXIN FAMILY |
| | | | | | | SIGNATURE |
| 1507 | PR00197B | 7.56 | 7.652e−17 | 47-69 | PR00197 | ANNEXIN TYPE I |
| | | | | | | SIGNATURE |
| 1507 | PR00197C | 7.50 | 2.394e−10 | 87-103 | PR00197 | ANNEXIN TYPE I |
| | | | | | | SIGNATURE |
| 1507 | PR00197D | 7.50 | 2.500e−09 | 274-295 | PR00197 | ANNEXIN TYPE I |
| | | | | | | SIGNATURE |
| 1507 | PR00197D | 7.50 | 6.906e−18 | 114-135 | PR00197 | ANNEXIN TYPE I |
| | | | | | | SIGNATURE |
| 1507 | PR00197E | 11.89 | 9.077e−15 | 198-224 | PR00197 | ANNEXIN TYPE I |
| | | | | | | SIGNATURE |
| 1507 | PR00197F | 9.03 | 2.561e−09 | 118-138 | PR00197 | ANNEXIN TYPE I |
| | | | | | | SIGNATURE |
| 1507 | PR00197F | 9.03 | 9.690e−21 | 278-298 | PR00197 | ANNEXIN TYPE I |
| | | | | | | SIGNATURE |
| 1507 | PR00197H | 11.98 | 1.988e−09 | 322-335 | PR00197 | ANNEXIN TYPE I |
| | | | | | | SIGNATURE |
| 1507 | PR00198A | 10.33 | 1.818e−17 | 3-15 | PR00198 | ANNEXIN TYPE II |
| | | | | | | SIGNATURE |
| 1507 | PR00198B | 8.71 | 3.842e−29 | 47-69 | PR00198 | ANNEXIN TYPE II |
| | | | | | | SIGNATURE |
| 1507 | PR00198C | 14.32 | 9.679e−20 | 87-103 | PR00198 | ANNEXIN TYPE II |
| | | | | | | SIGNATURE |
| 1507 | PR00198D | 7.65 | 2.206e−10 | 274-295 | PR00198 | ANNEXIN TYPE II |
| | | | | | | SIGNATURE |
| 1507 | PR00198D | 7.65 | 4.000e−28 | 114-135 | PR00198 | ANNEXIN TYPE II |
| | | | | | | SIGNATURE |
| 1507 | PR00198E | 14.67 | 8.364e−35 | 198-224 | PR00198 | ANNEXIN TYPE II |
| | | | | | | SIGNATURE |
| 1507 | PR00198F | 7.98 | 1.082e−10 | 254-261 | PR00198 | ANNEXIN TYPE II |
| | | | | | | SIGNATURE |
| 1507 | PR00198G | 8.09 | 6.250e−27 | 278-298 | PR00198 | ANNEXIN TYPE II |
| | | | | | | SIGNATURE |
| 1507 | PR00198G | 8.09 | 8.319e−10 | 118-138 | PR00198 | ANNEXIN TYPE II |
| | | | | | | SIGNATURE |
| 1507 | PR00198H | 12.05 | 1.429e−18 | 322-335 | PR00198 | ANNEXIN TYPE II |
| | | | | | | SIGNATURE |
| 1507 | PR00199B | 6.86 | 1.971e−11 | 119-141 | PR00199 | ANNEXIN TYPE III |
| | | | | | | SIGNATURE |
| 1507 | PR00199B | 6.86 | 5.605e−15 | 47-69 | PR00199 | ANNEXIN TYPE III |
| | | | | | | SIGNATURE |
| 1507 | PR00199B | 6.86 | 8.634e−09 | 204-226 | PR00199 | ANNEXIN TYPE III |
| | | | | | | SIGNATURE |
| 1507 | PR00199C | 13.84 | 9.786e−09 | 87-103 | PR00199 | ANNEXIN TYPE III |
| | | | | | | SIGNATURE |
| 1507 | PR00199D | 5.65 | 1.281e−17 | 114-135 | PR00199 | ANNEXIN TYPE III |
| | | | | | | SIGNATURE |
| 1507 | PR00199D | 5.65 | 4.931e−10 | 274-295 | PR00199 | ANNEXIN TYPE III |
| | | | | | | SIGNATURE |
| 1507 | PR00199F | 16.19 | 5.862e−09 | 113-139 | PR00199 | ANNEXIN TYPE III |
| | | | | | | SIGNATURE |
| 1507 | PR00199F | 16.19 | 7.750e−12 | 198-224 | PR00199 | ANNEXIN TYPE III |
| | | | | | | SIGNATURE |
| 1507 | PR00199G | 9.09 | 5.860e−10 | 119-144 | PR00199 | ANNEXIN TYPE III |
| | | | | | | SIGNATURE |
| 1507 | PR00199G | 9.09 | 9.645e−17 | 279-304 | PR00199 | ANNEXIN TYPE III |
| | | | | | | SIGNATURE |
| 1507 | PR00199H | 12.62 | 4.044e−09 | 322-335 | PR00199 | ANNEXIN TYPE III |
| | | | | | | SIGNATURE |
| 1507 | PR00200B | 7.39 | 5.099e−11 | 47-69 | PR00200 | ANNEXIN TYPE IV |
| | | | | | | SIGNATURE |
| 1507 | PR00200B | 7.39 | 9.465e−11 | 119-141 | PR00200 | ANNEXIN TYPE IV |
| | | | | | | SIGNATURE |
| 1507 | PR00200D | 10.01 | 7.554e−11 | 87-103 | PR00200 | ANNEXIN TYPE IV |
| | | | | | | SIGNATURE |
| 1507 | PR00200E | 10.00 | 2.111e−14 | 114-135 | PR00200 | ANNEXIN TYPE IV |
| | | | | | | SIGNATURE |
| 1507 | PR00200E | 10.00 | 5.825e−10 | 274-295 | PR00200 | ANNEXIN TYPE IV |
| | | | | | | SIGNATURE |
| 1507 | PR00200F | 13.72 | 9.100e−10 | 198-224 | PR00200 | ANNEXIN TYPE IV |
| | | | | | | SIGNATURE |
| 1507 | PR00200G | 9.43 | 4.038e−20 | 278-304 | PR00200 | ANNEXIN TYPE IV |
| | | | | | | SIGNATURE |
| 1507 | PR00200G | 9.43 | 6.143e−12 | 118-144 | PR00200 | ANNEXIN TYPE IV |
| | | | | | | SIGNATURE |
| 1507 | PR00200H | 13.68 | 2.753e−11 | 322-335 | PR00200 | ANNEXIN TYPE IV |
| | | | | | | SIGNATURE |
| 1507 | PR00200H | 13.68 | 3.118e−09 | 162-175 | PR00200 | ANNEXIN TYPE IV |
| | | | | | | SIGNATURE |
| 1507 | PR00201A | 6.05 | 3.069e−12 | 119-141 | PR00201 | ANNEXIN TYPE V |
| | | | | | | SIGNATURE |
| 1507 | PR00201A | 6.05 | 6.838e−16 | 47-69 | PR00201 | ANNEXIN TYPE V |
| | | | | | | SIGNATURE |
| 1507 | PR00201D | 10.49 | 1.495e−12 | 114-135 | PR00201 | ANNEXIN TYPE V |
| | | | | | | SIGNATURE |
| 1507 | PR00201D | 10.49 | 6.940e−10 | 274-295 | PR00201 | ANNEXIN TYPE V |
| | | | | | | SIGNATURE |
| 1507 | PR00201E | 12.37 | 8.129e−11 | 198-224 | PR00201 | ANNEXIN TYPE V |
| | | | | | | SIGNATURE |
| 1507 | PR00201G | 11.02 | 1.231e−19 | 278-304 | PR00201 | ANNEXIN TYPE V |
| | | | | | | SIGNATURE |
| 1507 | PR00201G | 11.02 | 7.888e−12 | 118-144 | PR00201 | ANNEXIN TYPE V |
| | | | | | | SIGNATURE |
| 1507 | PR00201H | 12.04 | 7.413e−13 | 322-335 | PR00201 | ANNEXIN TYPE V |
| | | | | | | SIGNATURE |
| 1507 | PR00202B | 11.44 | 1.287e−12 | 118-141 | PR00202 | ANNEXIN TYPE VI |
| | | | | | | SIGNATURE |
| 1507 | PR00202B | 11.44 | 7.680e−11 | 46-69 | PR00202 | ANNEXIN TYPE VI |
| | | | | | | SIGNATURE |
| 1507 | PR00202D | 5.58 | 7.028e−09 | 274-295 | PR00202 | ANNEXIN TYPE VI |
| | | | | | | SIGNATURE |
| 1507 | PR00202D | 5.58 | 9.242e−12 | 114-135 | PR00202 | ANNEXIN TYPE VI |
| | | | | | | SIGNATURE |
| 1507 | PR00202E | 13.00 | 9.400e−10 | 198-224 | PR00202 | ANNEXIN TYPE VI |
| | | | | | | SIGNATURE |
| 1507 | PR00202G | 8.01 | 5.417e−11 | 118-144 | PR00202 | ANNEXIN TYPE VI |
| | | | | | | SIGNATURE |
| 1507 | PR00202G | 8.01 | 8.606e−25 | 278-304 | PR00202 | ANNEXIN TYPE VI |
| | | | | | | SIGNATURE |
| 1507 | PR00202H | 9.20 | 5.050e−10 | 322-335 | PR00202 | ANNEXIN TYPE VI |
| | | | | | | SIGNATURE |
| 1508 | BL01144 | 25.07 | 1.000e−40 | 22-73 | BL01144 | Ribosomal protein L31e |
| | | | | | | proteins. |
| 1509 | PR00738A | 16.27 | 2.500e−20 | 158-178 | PR00738 | GLYCOSYL HYDROLASE |
| | | | | | | FAMILY 20 SIGNATURE |
| 1509 | PR00738B | 11.01 | 2.241e−18 | 194-211 | PR00738 | GLYCOSYL HYDROLASE |
| | | | | | | FAMILY 20 SIGNATURE |
| 1509 | PR00738C | 11.37 | 7.500e−26 | 223-244 | PR00738 | GLYCOSYL HYDROLASE |
| | | | | | | FAMILY 20 SIGNATURE |
| 1509 | PR00738D | 14.00 | 4.000e−21 | 277-294 | PR00738 | GLYCOSYL HYDROLASE |
| | | | | | | FAMILY 20 SIGNATURE |
| 1509 | PR00738E | 14.48 | 8.364e−16 | 322-340 | PR00738 | GLYCOSYL HYDROLASE |
| | | | | | | FAMILY 20 SIGNATURE |
| 1509 | PR00738F | 11.93 | 6.500e−15 | 344-357 | PR00738 | GLYCOSYL HYDROLASE |
| | | | | | | FAMILY 20 SIGNATURE |
| 1509 | PR00738G | 15.05 | 1.000e−19 | 481-497 | PR00738 | GLYCOSYL HYDROLASE |
| | | | | | | FAMILY 20 SIGNATURE |
| 1509 | PR00738H | 11.43 | 5.846e−18 | 498-515 | PR00738 | GLYCOSYL HYDROLASE |
| | | | | | | FAMILY 20 SIGNATURE |
| 1510 | BL00854A | 33.93 | 1.818e−26 | 58-103 | BL00854 | Proteasome B-type |
| | | | | | | subunits proteins. |
| 1510 | BL00854C | 29.92 | 8.435e−19 | 168-196 | BL00854 | Proteasome B-type |
| | | | | | | subunits proteins. |
| 1511 | BL00487A | 8.84 | 2.125e−12 | 30-39 | BL00487 | IMP dehydrogenase/GMP |
| | | | | | | reductase proteins. |
| 1511 | BL00487B | 16.59 | 1.000e−40 | 61-98 | BL00487 | IMP dehydrogenase/GMP |
| | | | | | | reductase proteins. |
| 1511 | BL00487C | 10.90 | 8.043e−13 | 153-165 | BL00487 | IMP dehydrogenase/GMP |
| | | | | | | reductase proteins. |
| 1511 | BL00487D | 26.53 | 1.000e−40 | 242-294 | BL00487 | IMP dehydrogenase/GMP |
| | | | | | | reductase proteins. |
| 1511 | BL00487E | 16.12 | 1.000e−40 | 299-343 | BL00487 | IMP dehydrogenase/GMP |
| | | | | | | reductase proteins. |
| 1511 | BL00487F | 18.79 | 1.000e−40 | 380-420 | BL00487 | IMP dehydrogenase/GMP |
| | | | | | | reductase proteins. |
| 1511 | BL00487G | 26.82 | 9.500e−38 | 439-480 | BL00487 | IMP dehydrogenase/GMP |
| | | | | | | reductase proteins. |
| 1511 | BL00487H | 15.21 | 3.368e−17 | 490-506 | BL00487 | IMP dehydrogenase/GMP |
| | | | | | | reductase proteins. |
| 1511 | BL00557D | 17.76 | 1.297e−12 | 361-402 | BL00557 | FMN-dependent alpha- |
| | | | | | | hydroxy acid |
| | | | | | | dehydrogenases |
| | | | | | | proteins. |
| 1511 | BL00911G | 19.64 | 3.654e−09 | 359-389 | BL00911 | Dihydroorotate |
| | | | | | | dehydrogenase proteins. |
| 1513 | PR00019A | 11.19 | 4.000e−09 | 207-220 | PR00019 | LEUCINE-RICH REPEAT |
| | | | | | | SIGNATURE |
| 1513 | PR00019B | 11.36 | 1.720e−09 | 230-243 | PR00019 | LEUCINE-RICH REPEAT |
| | | | | | | SIGNATURE |
| 1513 | PR00019B | 11.36 | 9.640e−09 | 204-217 | PR00019 | LEUCINE-RICH REPEAT |
| | | | | | | SIGNATURE |
| 1514 | BL00019D | 15.33 | 3.520e−17 | 818-847 | BL00019 | Actinin-type actin- |
| | | | | | | binding domain |
| | | | | | | proteins. |
| 1514 | PD01351B | 13.72 | 4.981e−11 | 233-258 | PD01351 | PROTEIN REPEAT |
| | | | | | | NEUROFILAMENT TRIPL. |
| 1514 | PF00992A | 16.67 | 2.895e−09 | 607-641 | PF00992 | Troponin. |
| 1516 | BL00417A | 7.74 | 6.704e−13 | 5-32 | BL00417 | Synaptobrevin proteins. |
| 1516 | BL00417B | 18.48 | 2.414e−19 | 33-86 | BL00417 | Synaptobrevin proteins. |
| 1516 | PR00219A | 8.98 | 9.156e−13 | 10-29 | PR00219 | SYNAPTOBREVIN SIGNATURE |
| 1518 | BL00191J | 11.37 | 6.447e−17 | 128-149 | BL00191 | Cytochrome b5 family, |
| | | | | | | heme-binding domain |
| | | | | | | proteins. |
| 1518 | BL00191K | 17.38 | 4.951e−27 | 184-227 | BL00191 | Cytochrome b5 family, |
| | | | | | | heme-binding domain |
| | | | | | | proteins. |
| 1518 | BL00559I | 13.63 | 7.366e−21 | 58-86 | BL00559 | Eukaryotic |
| | | | | | | molybdopterin |
| | | | | | | oxidoreductases |
| | | | | | | proteins. |
| 1518 | BL00559J | 19.63 | 9.348e−16 | 128-179 | BL00559 | Eukaryotic |
| | | | | | | molybdopterin |
| | | | | | | oxidoreductases |
| | | | | | | proteins. |
| 1518 | BL00559K | 13.17 | 7.563e−17 | 201-227 | BL00559 | Eukaryotic |
| | | | | | | molybdopterin |
| | | | | | | oxidoreductases |
| | | | | | | proteins. |
| 1518 | BL00559L | 13.60 | 1.514e−14 | 270-287 | BL00559 | Eukaryotic |
| | | | | | | molybdopterin |
| | | | | | | oxidoreductases |
| | | | | | | proteins. |
| 1518 | PR00371D | 14.55 | 2.313e−13 | 180-199 | PR00371 | FLAVOPROTEIN PYRIDINE |
| | | | | | | NUCLEOTIDE CYTOCHROME |
| | | | | | | REDUCTASE SIGNATURE |
| 1518 | PR00406A | 11.92 | 4.375e−13 | 75-86 | PR00406 | CYTOCHROME B5 REDUCTASE |
| | | | | | | SIGNATURE |
| 1518 | PR00406C | 13.10 | 7.750e−12 | 141-155 | PR00406 | CYTOCHROME B5 REDUCTASE |
| | | | | | | SIGNATURE |
| 1518 | PR00406D | 10.02 | 7.387e−19 | 180-199 | PR00406 | CYTOCHROME B5 REDUCTASE |
| | | | | | | SIGNATURE |
| 1518 | PR00406E | 8.37 | 9.400e−09 | 218-229 | PR00406 | CYTOCHROME B5 REDUCTASE |
| | | | | | | SIGNATURE |
| 1518 | PR00406F | 3.97 | 4.240e−10 | 274-282 | PR00406 | CYTOCHROME B5 REDUCTASE |
| | | | | | | SIGNATURE |
| 1518 | PR00409F | 12.70 | 6.612e−10 | 180-199 | PR00409 | PHTHALATE DIOXYGENASE |
| | | | | | | REDUCTASE FAMILY |
| | | | | | | SIGNATURE |
| 1518 | PR00410D | 12.97 | 4.676e−09 | 180-199 | PR00410 | PHENOL HYDROXYLASE |
| | | | | | | REDUCTASE FAMILY |
| | | | | | | SIGNATURE |
| 1519 | BL00297A | 17.58 | 1.000e−40 | 7-43 | BL00297 | Heat shock hsp70 |
| | | | | | | proteins family |
| | | | | | | proteins. |
| 1519 | BL00297B | 22.91 | 1.000e−40 | 46-94 | BL00297 | Heat shock hsp70 |
| | | | | | | proteins family |
| | | | | | | proteins. |
| 1519 | BL00297C | 9.51 | 1.000e−40 | 134-185 | BL00297 | Heat shock hsp70 |
| | | | | | | proteins family |
| | | | | | | proteins. |
| 1519 | BL00297D | 11.95 | 1.000e−40 | 195-234 | BL00297 | Heat shock hsp70 |
| | | | | | | proteins family |
| | | | | | | proteins. |
| 1519 | BL00297E | 18.56 | 8.800e−40 | 292-335 | BL00297 | Heat shock hsp70 |
| | | | | | | proteins family |
| | | | | | | proteins. |
| 1519 | BL00297F | 11.84 | 6.400e−29 | 356-383 | BL00297 | Heat shock hsp70 |
| | | | | | | proteins family |
| | | | | | | proteins. |
| 1519 | BL00297G | 21.87 | 1.000e−40 | 390-444 | BL00297 | Heat shock hsp70 |
| | | | | | | proteins family |
| | | | | | | proteins. |
| 1519 | BL00297H | 15.46 | 1.000e−40 | 453-506 | BL00297 | Heat shock hsp70 |
| | | | | | | proteins family |
| | | | | | | proteins. |
| 1519 | PR00301A | 14.84 | 3.368e−16 | 5-18 | PR00301 | 70 KD HEAT SHOCK |
| | | | | | | PROTEIN SIGNATURE |
| 1519 | PR00301B | 15.36 | 1.692e−17 | 33-45 | PR00301 | 70 KD HEAT SHOCK |
| | | | | | | PROTEIN SIGNATURE |
| 1519 | PR00301C | 8.62 | 9.609e−10 | 55-63 | PR00301 | 70 KD HEAT SHOCK |
| | | | | | | PROTEIN SIGNATURE |
| 1519 | PR00301D | 15.51 | 5.500e−27 | 142-162 | PR00301 | 70 KD HEAT SHOCK |
| | | | | | | PROTEIN SIGNATURE |
| 1519 | PR00301E | 8.11 | 2.286e−14 | 203-213 | PR00301 | 70 KD HEAT SHOCK |
| | | | | | | PROTEIN SIGNATURE |
| 1519 | PR00301F | 13.98 | 1.429e−19 | 331-347 | PR00301 | 70 KD HEAT SHOCK |
| | | | | | | PROTEIN SIGNATURE |
| 1519 | PR00301G | 13.78 | 6.143e−25 | 363-383 | PR00301 | 70 KD HEAT SHOCK |
| | | | | | | PROTEIN SIGNATURE |
| 1519 | PR00301H | 8.81 | 2.421e−21 | 390-409 | PR00301 | 70 KD HEAT SHOCK |
| | | | | | | PROTEIN SIGNATURE |
| 1519 | PR00301I | 12.76 | 2.125e−22 | 471-487 | PR00301 | 70 KD HEAT SHOCK |
| | | | | | | PROTEIN SIGNATURE |
| 1520 | BL00960B | 24.47 | 9.471e−09 | 195-239 | BL00960 | BTG1 family proteins. |
| 1520 | PR00738A | 16.27 | 6.538e−17 | 125-145 | PR00738 | GLYCOSYL HYDROLASE |
| | | | | | | FAMILY 20 SIGNATURE |
| 1520 | PR00738B | 11.01 | 1.000e−18 | 161-178 | PR00738 | GLYCOSYL HYDROLASE |
| | | | | | | FAMILY 20 SIGNATURE |
| 1520 | PR00738C | 11.37 | 3.483e−23 | 190-211 | PR00738 | GLYCOSYL HYDROLASE |
| | | | | | | FAMILY 20 SIGNATURE |
| 1520 | PR00738D | 14.00 | 4.194e−19 | 245-262 | PR00738 | GLYCOSYL HYDROLASE |
| | | | | | | FAMILY 20 SIGNATURE |
| 1520 | PR00738E | 14.48 | 5.500e−16 | 290-308 | PR00738 | GLYCOSYL HYDROLASE |
| | | | | | | FAMILY 20 SIGNATURE |
| 1520 | PR00738F | 11.93 | 3.455e−14 | 312-325 | PR00738 | GLYCOSYL HYDROLASE |
| | | | | | | FAMILY 20 SIGNATURE |
| 1520 | PR00738G | 15.05 | 1.000e−20 | 452-468 | PR00738 | GLYCOSYL HYDROLASE |
| | | | | | | FAMILY 20 SIGNATURE |
| 1520 | PR00738H | 11.43 | 9.308e−18 | 469-486 | PR00738 | GLYCOSYL HYDROLASE |
| | | | | | | FAMILY 20 SIGNATURE |
| 1521 | BL00030B | 7.03 | 2.200e−10 | 103-112 | BL00030 | Eukaryotic RNA-binding |
| | | | | | | region RNP-1 proteins. |
| 1522 | BL00540A | 15.06 | 8.636e−40 | 5-45 | BL00540 | Ferritin iron-binding |
| | | | | | | regions proteins. |
| 1522 | BL00540B | 18.82 | 1.000e−40 | 96-150 | BL00540 | Ferritin iron-binding |
| | | | | | | regions proteins. |
| 1522 | BL00540C | 13.00 | 4.649e−12 | 161-172 | BL00540 | Ferritin iron-binding |
| | | | | | | regions proteins. |
| 1524 | BL00362 | 24.67 | 3.348e−35 | 88-130 | BL00362 | Ribosomal protein S15 |
| | | | | | | proteins. |
| 1525 | BL00406A | 9.95 | 1.000e−40 | 9-43 | BL00406 | Actins proteins. |
| 1525 | BL00406B | 5.47 | 1.000e−40 | 85-139 | BL00406 | Actins proteins. |
| 1525 | BL00406C | 6.75 | 1.000e−40 | 144-198 | BL00406 | Actins proteins. |
| 1525 | BL00406D | 12.58 | 1.000e−40 | 271-325 | BL00406 | Actins proteins. |
| 1525 | BL00406E | 8.44 | 1.000e−40 | 328-377 | BL00406 | Actins proteins. |
| 1525 | PR00190A | 7.24 | 1.375e−13 | 28-37 | PR00190 | ACTIN SIGNATURE |
| 1525 | PR00190B | 9.98 | 1.000e−15 | 51-62 | PR00190 | ACTIN SIGNATURE |
| 1525 | PR00190C | 11.49 | 4.789e−30 | 63-85 | PR00190 | ACTIN SIGNATURE |
| 1525 | PR00150D | 19.23 | 3.520e−24 | 86-104 | PR00190 | ACTIN SIGNATURE |
| 1525 | PR00190E | 7.16 | 1.450e−19 | 117-130 | PR00190 | ACTIN SIGNATURE |
| 1525 | PR00190F | 7.80 | 1.321e−24 | 142-161 | PR00190 | ACTIN SIGNATURE |
| 1525 | PR00190G | 12.62 | 1.600e−21 | 238-254 | PR00190 | ACTIN SIGNATURE |
| 1526 | BL00798A | 14.97 | 5.200e−17 | 7-21 | BL00798 | Aldo/keto reductase |
| | | | | | | family proteins. |
| 1526 | BL00798B | 16.01 | 5.500e−28 | 35-59 | BL00798 | Aldo/keto reductase |
| | | | | | | family proteins. |
| 1526 | BL00798C | 11.15 | 7.480e−15 | 68-80 | BL00798 | Aldo/keto reductase |
| | | | | | | family proteins. |
| 1526 | BL00798D | 7.65 | 5.235e−17 | 92-108 | BL00798 | Aldo/keto reductase |
| | | | | | | family proteins. |
| 1526 | BL00798E | 20.32 | 1.000e−40 | 177-214 | BL00798 | Aldo/keto reductase |
| | | | | | | family proteins. |
| 1526 | BL00798F | 23.30 | 1.000e−40 | 238-286 | BL00798 | Aldo/keto reductase |
| | | | | | | family proteins. |
| 1526 | PR00069A | 16.01 | 8.364e−29 | 35-59 | PR00069 | ALDO-KETO REDUCTASE |
| | | | | | | SIGNATURE |
| 1526 | PR00069B | 11.33 | 1.000e−21 | 95-113 | PR00069 | ALDO-KETO REDUCTASE |
| | | | | | | SIGNATURE |
| 1526 | PR00069C | 16.03 | 4.000e−19 | 145-162 | PR00069 | ALDO-KETO REDUCTASE |
| | | | | | | SIGNATURE |
| 1526 | PR00069D | 19.36 | 1.529e−32 | 181-210 | PR00069 | ALDO-KETO REDUCTASE |
| | | | | | | SIGNATURE |
| 1526 | PR00069E | 18.14 | 6.870e−25 | 228-252 | PR00069 | ALDO-KETO REDUCTASE |
| | | | | | | SIGNATURE |
| 1527 | BL00086 | 20.87 | 2.421e−21 | 427-458 | BL00086 | Cytochrome P450 |
| | | | | | | cysteine heme-iron |
| | | | | | | ligand proteins. |
| 1527 | PR00385A | 14.97 | 8.448e−11 | 306-323 | PR00385 | P450 SUPERFAMILY |
| | | | | | | SIGNATURE |
| 1527 | PR00385B | 10.22 | 4.300e−09 | 324-337 | PR00385 | P450 SUPERFAMILY |
| | | | | | | SIGNATURE |
| 1527 | PR00385D | 13.11 | 4.000e−09 | 428-437 | PR00385 | P450 SUPERFAMILY |
| | | | | | | SIGNATURE |
| 1527 | PR00385E | 12.66 | 6.571e−09 | 437-448 | PR00385 | P450 SUPERFAMILY |
| | | | | | | SIGNATURE |
| 1527 | PR00463E | 17.37 | 9.542e−11 | 315-341 | PR00463 | E-CLASS P450 GROUP I |
| | | | | | | SIGNATURE |
| 1527 | PR00464E | 18.28 | 4.696e−09 | 352-372 | PR00464 | E-CLASS P450 GROUP II |
| | | | | | | SIGNATURE |
| 1527 | PR00464I | 14.64 | 9.654e−09 | 437-460 | PR00464 | E-CLASS P450 GROUP II |
| | | | | | | SIGNATURE |
| 1528 | PR00049D | 0.00 | 9.929e−10 | 180-194 | PR00049 | WILM'S TUMOUR PROTEIN |
| | | | | | | SIGNATURE |
| 1529 | BL00678 | 9.67 | 1.692e−11 | 80-90 | BL00678 | Trp-Asp (WD) repeat |
| | | | | | | proteins proteins. |
| 1529 | BL00678 | 9.67 | 1.947e−09 | 167-177 | BL00678 | Trp-Asp (WD) repeat |
| | | | | | | proteins proteins. |
| 1529 | BL00678 | 9.67 | 8.615e−11 | 122-132 | BL00678 | Trp-Asp (WD) repeat |
| | | | | | | proteins proteins. |
| 1529 | PR00320A | 16.74 | 1.439e−09 | 207-221 | PR00320 | G-PROTEIN BETA WD-40 |
| | | | | | | REPEAT SIGNATURE |
| 1529 | PR00320A | 16.74 | 3.143e−13 | 78-92 | PR00320 | G-PROTEIN BETA WD-40 |
| | | | | | | REPEAT SIGNATURE |
| 1529 | PR00320A | 16.74 | 3.382e−10 | 31-45 | PR00320 | G-PROTEIN BETA WD-40 |
| | | | | | | REPEAT SIGNATURE |
| 1529 | PR00320A | 16.74 | 7.207e−11 | 165-179 | PR00320 | G-PROTEIN BETA WD-40 |
| | | | | | | REPEAT SIGNATURE |
| 1529 | PR00320A | 16.74 | 8.560e−12 | 120-134 | PR00320 | G-PROTEIN BETA WD-40 |
| | | | | | | REPEAT SIGNATURE |
| 1529 | PR00320B | 12.19 | 1.257e−10 | 31-45 | PR00320 | G-PROTEIN BETA WD-40 |
| | | | | | | REPEAT SIGNATURE |
| 1529 | PR00320B | 12.19 | 2.174e−13 | 120-134 | PR00320 | G-PROTEIN BETA WD-40 |
| | | | | | | REPEAT SIGNATURE |
| 1529 | PR00320B | 12.19 | 4.484e−11 | 165-179 | PR00320 | G-PROTEIN BETA WD-40 |
| | | | | | | REPEAT SIGNATURE |
| 1529 | PR00320B | 12.19 | 4.825e−09 | 207-221 | PR00320 | G-PROTEIN BETA WD-40 |
| | | | | | | REPEAT SIGNATURE |
| 1529 | PR00320B | 12.19 | 7.231e−12 | 78-92 | PR00320 | G-PROTEIN BETA WD-40 |
| | | | | | | REPEAT SIGNATURE |
| 1529 | PR00320C | 13.01 | 1.000e−10 | 78-92 | PR00320 | G-PROTEIN BETA WD-40 |
| | | | | | | REPEAT SIGNATURE |
| 1529 | PR00320C | 13.01 | 2.800e−10 | 120-134 | PR00320 | G-PROTEIN BETA WD-40 |
| | | | | | | REPEAT SIGNATURE |
| 1529 | PR00320C | 13.01 | 7.000e−12 | 207-221 | PR00320 | G-PROTEIN BETA WD-40 |
| | | | | | | REPEAT SIGNATURE |
| 1529 | PR00320C | 13.01 | 8.043e−11 | 165-179 | PR00320 | G-PROTEIN BETA WD-40 |
| | | | | | | REPEAT SIGNATURE |
| 1529 | PR00320C | 13.01 | 9.700e−09 | 31-45 | PR00320 | G-PROTEIN BETA WD-40 |
| | | | | | | REPEAT SIGNATURE |
| 1530 | BL01082 | 20.37 | 5.000e−34 | 51-90 | BL01082 | Ribosomal protein L7Ae |
| | | | | | | proteins. |
| 1530 | PR00881A | 9.94 | 3.455e−12 | 35-49 | PR00881 | RIBOSOMAL PROTEIN |
| | | | | | | L7A/RS6 FAMILY |
| | | | | | | SIGNATURE |
| 1530 | PR00881B | 9.01 | 4.484e−12 | 54-67 | PR00881 | RIBOSOMAL PROTEIN |
| | | | | | | L7A/RS6 FAMILY |
| | | | | | | SIGNATURE |
| 1530 | PR00881C | 12.76 | 9.526e−13 | 70-80 | PR00881 | RIBOSOMAL PROTEIN |
| | | | | | | L7A/RS6 FAMILY |
| | | | | | | SIGNATURE |
| 1530 | PR00881D | 11.60 | 2.440e−14 | 80-94 | PR00881 | RIBOSOMAL PROTEIN |
| | | | | | | L7A/RS6 FAMILY |
| | | | | | | SIGNATURE |
| 1530 | PR00883A | 6.49 | 5.500e−18 | 8-21 | PR00883 | HIGH MOBILITY GROUP- |
| | | | | | | LIKE NUCLEAR PROTEIN |
| | | | | | | SIGNATURE |
| 1530 | PR00883B | 8.76 | 8.364e−19 | 21-36 | PR00883 | HIGH MOBILITY GROUP- |
| | | | | | | LIKE NUCLEAR PROTEIN |
| | | | | | | SIGNATURE |
| 1530 | PR00883C | 8.52 | 2.000e−14 | 38-48 | PR00883 | HIGH MOBILITY GROUP- |
| | | | | | | LIKE NUCLEAR PROTEIN |
| | | | | | | SIGNATURE |
| 1530 | PR00883D | 13.33 | 2.200e−16 | 82-1694 | PR00883 | HIGH MOBILITY GROUP- |
| | | | | | | LIKE NUCLEAR PROTEIN |
| | | | | | | SIGNATURE |
| 1530 | PR00884D | 11.07 | 9.217e−10 | 80-90 | PR00884 | RIBOSOMAL PROTEIN HS6 |
| | | | | | | SIGNATURE |
| 1531 | BL00296A | 17.20 | 5.648e−10 | 12-65 | BL00296 | Chaperonins cpn60 |
| | | | | | | proteins. |
| 1531 | BL00296B | 15.98 | 4.115e−13 | 76-129 | BL00296 | Chaperonins cpn60 |
| | | | | | | proteins. |
| 1531 | BL00296F | 19.66 | 3.826e−09 | 476-513 | BL00296 | Chaperonins cpn60 |
| | | | | | | proteins. |
| 1531 | BL00750A | 20.07 | 8.286e−36 | 25-67 | BL00750 | Chaperonins TCP-1 |
| | | | | | | proteins. |
| 1531 | BL00750B | 16.17 | 2.000e−39 | 69-118 | BL00750 | Chaperonins TCP-1 |
| | | | | | | proteins. |
| 1531 | BL00750C | 25.65 | 8.579e−23 | 152-183 | BL00750 | Chaperonins TCP-1 |
| | | | | | | proteins. |
| 1531 | BL00750D | 16.16 | 5.875e−13 | 199-217 | BL00750 | Chaperonins TCP-1 |
| | | | | | | proteins. |
| 1531 | BL00750E | 24.59 | 8.313e−21 | 294-330 | BL00750 | Chaperonins TCP-1 |
| | | | | | | proteins. |
| 1531 | BL00750F | 18.40 | 3.880e−29 | 368-408 | BL00750 | Chaperonins TCP-1 |
| | | | | | | proteins. |
| 1531 | BL00750G | 20.12 | 4.300e−31 | 429-468 | BL00750 | Chaperonins TCP-1 |
| | | | | | | proteins. |
| 1531 | BL00750H | 21.44 | 2.731e−23 | 486-520 | BL00750 | Chaperonins TCP-1 |
| | | | | | | proteins. |
| 1531 | PR00298B | 13.59 | 7.353e−11 | 88-115 | PR00298 | 60 KD CHAPERONIN |
| | | | | | | SIGNATURE |
| 1531 | PR00304A | 9.20 | 3.605e−15 | 34-50 | PR00304 | TAILLESS COMPLEX |
| | | | | | | POLYPEPTIDE 1 |
| | | | | | | (CHAPERONE) SIGNATURE |
| 1531 | PR00304B | 11.60 | 2.059e−17 | 56-74 | PR00304 | TAILLESS COMPLEX |
| | | | | | | POLYPEPTIDE 1 |
| | | | | | | (CHAPERONE) SIGNATURE |
| 1531 | PR00304C | 8.69 | 1.250e−18 | 86-105 | PR00304 | TAILLESS COMPLEX |
| | | | | | | POLYPEPTIDE 1 |
| | | | | | | (CHAPERONE) SIGNATURE |
| 1531 | PR00304D | 11.04 | 9.757e−17 | 371-393 | PR00304 | TAILLESS COMPLEX |
| | | | | | | POLYPEPTIDE 1 |
| | | | | | | (CHAPERONE) SIGNATURE |
| 1531 | PR00304E | 7.79 | 8.043e−13 | 405-417 | PR00304 | TAILLESS COMPLEX |
| | | | | | | POLYPEPTIDE 1 |
| | | | | | | (CHAPERONE) SIGNATURE |
| 1532 | BL00021B | 13.33 | 2.435e−10 | 487-504 | BL00021 | Kringle domain |
| | | | | | | proteins. |
| 1532 | BL00021D | 24.56 | 1.105e−09 | 656-697 | BL00021 | Kringle domain |
| | | | | | | proteins. |
| 1532 | BL00022B | 7.54 | 9.100e−09 | 174-180 | BL00022 | EGF-like domain |
| | | | | | | proteins. |
| 1532 | BL00134A | 11.96 | 5.114e−14 | 487-503 | BL00134 | Serine proteases, |
| | | | | | | trypsin family, |
| | | | | | | histidine proteins. |
| 1532 | BL00134B | 15.99 | 3.382e−13 | 648-671 | BL00134 | Serine proteases, |
| | | | | | | trypsin family, |
| | | | | | | histidine proteins. |
| 1532 | BL00134C | 13.45 | 5.500e−12 | 684-697 | BL00134 | Serine proteases, |
| | | | | | | trypsin family, |
| | | | | | | histidine proteins. |
| 1532 | BL004950 | 13.75 | 6.040e−09 | 675-703 | BL00495 | Apple domain proteins. |
| 1532 | BL01187B | 12.04 | 6.538e−16 | 165-180 | BL01187 | Calcium-binding EGF- |
| | | | | | | like domain proteins |
| | | | | | | pattern proteins. |
| 1532 | BL01253G | 11.34 | 5.382e−11 | 647-660 | BL01253 | Type I fibronectin |
| | | | | | | domain proteins. |
| 1532 | BL01253H | 13.15 | 9.587e−09 | 666-700 | BL01253 | Type I fibronectin |
| | | | | | | domain proteins. |
| 1532 | PR00010C | 11.16 | 3.571e−09 | 170-180 | PR00010 | TYPE II EGF-LIKE |
| | | | | | | SIGNATURE |
| 1532 | PR00722A | 12.27 | 8.258e−13 | 488-503 | PR00722 | CHYMOTRYPSIN SERINE |
| | | | | | | PROTEASE FAMILY (S1) |
| | | | | | | SIGNATURE |
| 1532 | PR00722B | 12.51 | 3.625e−09 | 553-567 | PR00722 | CHYMOTRYPSIN SERINE |
| | | | | | | PROTEASE FAMILY (S1) |
| | | | | | | SIGNATURE |
| 1532 | PR00722C | 10.87 | 8.105e−12 | 647-659 | PR00722 | CHYMOTRYPSIN SERINE |
| | | | | | | PROTEASE FAMILY (S1) |
| | | | | | | SIGNATURE |
| 1533 | BL00021B | 13.33 | 5.431e−11 | 460-477 | BL00021 | Kringle domain |
| | | | | | | proteins. |
| 1533 | BL00134A | 11.96 | 4.094e−15 | 460-476 | BL00134 | Serine proteases, |
| | | | | | | trypsin family, |
| | | | | | | histidine proteins. |
| 1533 | BL00134B | 15.99 | 5.378e−12 | 626-649 | BL00134 | Serine proteases, |
| | | | | | | trypsin family, |
| | | | | | | histidine proteins. |
| 1533 | BL00134C | 13.45 | 5.125e−12 | 662-675 | BL00134 | Serine proteases, |
| | | | | | | trypsin family, |
| | | | | | | histidine proteins. |
| 1533 | BL00495K | 12.58 | 4.845e−09 | 462-494 | BL00495 | Apple domain proteins. |
| 1533 | BL01180B | 11.58 | 2.875e−09 | 65-75 | BL01180 | CUB domain proteins |
| | | | | | | profile. |
| 1533 | BL01187B | 12.04 | 9.550e−14 | 147-162 | BL01187 | Calcium-binding EGF- |
| | | | | | | like domain proteins |
| | | | | | | pattern proteins. |
| 1533 | BL01253D | 4.84 | 7.960e−09 | 460-473 | BL01253 | Type I fibronectin |
| | | | | | | domain proteins. |
| 1533 | PF00084B | 9.45 | 8.875e−10 | 379-390 | PF00084 | Sushi domain proteins |
| | | | | | | (SCR repeat proteins. |
| 1533 | PR00722A | 12.27 | 7.120e−15 | 461-476 | PR00722 | CHYMOTRYPSIN SERINE |
| | | | | | | PROTEASE FAMILY (S1) |
| | | | | | | SIGNATURE |
| 1533 | PR00722B | 12.51 | 6.625e−12 | 525-539 | PR00722 | CHYMOTRYPSIN SERINE |
| | | | | | | PROTEASE FAMILY (S1) |
| | | | | | | SIGNATURE |
| 1533 | PR00722C | 10.87 | 3.864e−11 | 625-637 | PR00722 | CHYMOTRYPSIN SERINE |
| | | | | | | PROTEASE FAMILY (S1) |
| | | | | | | SIGNATURE |
| 1533 | PR00907B | 11.29 | 8.364e−10 | 143-159 | PR00907 | THROMBOMODULIN |
| | | | | | | SIGNATURE |
| 1535 | BL00018 | 7.41 | 1.000e−09 | 57-69 | BL00018 | EF-hand calcium-binding |
| | | | | | | domain proteins. |
| 1535 | BL00018 | 7.41 | 3.400e−12 | 130-142 | BL00018 | EF-hand calcium-binding |
| | | | | | | domain proteins. |
| 1535 | BL00303B | 26.15 | 6.344e−10 | 45-81 | BL00303 | S-100/ICaBP type |
| | | | | | | calcium binding |
| | | | | | | protein. |
| 1535 | PR00450C | 12.22 | 4.480e−10 | 89-110 | PR00450 | RECOVERIN FAMILY |
| | | | | | | SIGNATURE |
| 1535 | PR00450C | 12.22 | 5.320e−10 | 16-37 | PR00450 | RECOVERIN FAMILY |
| | | | | | | SIGNATURE |
| 1535 | PR00450C | 12.22 | 6.520e−10 | 52-73 | PR00450 | RECOVERIN FAMILY |
| | | | | | | SIGNATURE |
| 1536 | BL00319C | 17.12 | 9.289e−09 | 383-416 | BL00319 | Amyloidogenic |
| | | | | | | glycoprotein |
| | | | | | | extracellular domain |
| | | | | | | proteins. |
| 1536 | BL00412D | 16.54 | 2.565e−10 | 364-414 | BL00412 | Neuromodulin (GAP-43) |
| | | | | | | proteins. |
| 1536 | BL00412D | 16.54 | 2.929e−09 | 350-400 | BL00412 | Neuromodulin (GAP-43) |
| | | | | | | proteins. |
| 1536 | BL00412D | 16.54 | 4.398e−09 | 348-398 | BL00412 | Neuromodulin (GAP-43) |
| | | | | | | proteins. |
| 1536 | BL00412D | 16.54 | 8.714e−09 | 354-404 | BL00412 | Neuromodulin (GAP-43) |
| | | | | | | proteins. |
| 1536 | BL00412D | 16.54 | 9.265e−09 | 339-389 | BL00412 | Neuromodulin (GAP-43) |
| | | | | | | proteins. |
| 1536 | BL00422C | 16.18 | 1.176e−09 | 384-411 | BL00422 | Granins proteins. |
| 1536 | BL00422C | 16.18 | 8.059e−09 | 376-403 | BL00422 | Granins proteins. |
| 1536 | BL00803A | 14.83 | 2.688e−14 | 35-48 | BL00803 | Calreticulin family |
| | | | | | | proteins. |
| 1536 | BL00803B | 17.08 | 8.714e−18 | 63-81 | BL00803 | Calreticulin family |
| | | | | | | proteins. |
| 1536 | BL00803C | 11.13 | 6.063e−26 | 91-113 | BL00803 | Calreticulin family |
| | | | | | | proteins. |
| 1536 | BL00803D | 16.08 | 1.000e−15 | 128-138 | BL00803 | Calreticulin family |
| | | | | | | proteins. |
| 1536 | BL00803E | 16.55 | 2.588e−31 | 166-196 | BL00803 | Calreticulin family |
| | | | | | | proteins. |
| 1536 | BL00803F | 10.95 | 2.000e−37 | 225-255 | BL00803 | Calreticulin family |
| | | | | | | proteins. |
| 1536 | BL00803F | 10.95 | 2.179e−11 | 191-221 | BL00803 | Calreticulin family |
| | | | | | | proteins. |
| 1536 | BL00803F | 10.95 | 7.268e−22 | 208-238 | BL00803 | Calreticulin family |
| | | | | | | proteins. |
| 1536 | BL00803F | 10.95 | 9.516e−9 | 242-272 | BL00803 | Calreticulin family |
| 1536 | BL00803G | 14.33 | 1.000e−40 | 258-302 | BL00803 | Calreticulin family |
| | | | | | | proteins. |
| 1536 | BL00803G | 14.33 | 1.127e−19 | 244-288 | BL00803 | Calreticulin family |
| | | | | | | proteins. |
| 1536 | BL00803G | 14.33 | 7.429e−16 | 272-316 | BL00803 | Calreticulin family |
| | | | | | | proteins. |
| 1536 | BL00803H | 16.02 | 4.000e−10 | 309-325 | BL00803 | Calreticulin family |
| | | | | | | proteins. |
| 1536 | BL00803H | 16.02 | 7.375e−14 | 337-353 | BL00803 | Calreticulin family |
| | | | | | | proteins. |
| 1536 | PD00301B | 5.49 | 6.538e−09 | 371-381 | PD00301 | PROTEIN REPEAT MUSCLE |
| | | | | | | CALCIUM-BI. |
| 1536 | PF00992A | 16.67 | 8.986e−10 | 363-397 | PF00992 | Troponin. |
| 1536 | PR00626A | 14.35 | 1.500e−18 | 100-118 | PR00626 | CALRETICULIN SIGNATURE |
| 1536 | PR00626B | 14.12 | 2.200e−20 | 126-142 | PR00626 | CALRETICULIN SIGNATURE |
| 1536 | PR00626C | 9.70 | 7.882e−14 | 232-245 | PR00626 | CALRETICULIN SIGNATURE |
| 1536 | PR00626C | 9.70 | 9.100e−18 | 215-228 | PR00626 | CALRETICULIN SIGNATURE |
| 1536 | PR00626D | 8.30 | 6.520e−09 | 208-230 | PR00626 | CALRETICULIN SIGNATURE |
| 1536 | PR00626D | 8.30 | 8.017e−13 | 256-278 | PR00626 | CALRETICULIN SIGNATURE |
| 1536 | PR00626D | 8.30 | 8.071e−30 | 242-264 | PR00626 | CALRETICULIN SIGNATURE |
| 1536 | PR00626E | 11.30 | 3.676e−19 | 266-285 | PR00626 | CALRETICULIN SIGNATURE |
| 1536 | PR00626E | 11.30 | 7.632e−24 | 280-299 | PR00626 | CALRETICULIN SIGNATURE |
| 1536 | PR00626F | 15.79 | 5.500e−21 | 313-333 | PR00626 | CALRETICULIN SIGNATURE |
| 1537 | BL00303A | 21.77 | 5.286e−30 | 4-40 | BL00303 | S-100/ICaBP type |
| | | | | | | calcium binding |
| | | | | | | protein. |
| 1537 | BL00303B | 26.15 | 1.000e−29 | 51-87 | BL00303 | S-100/ICaBP type |
| | | | | | | calcium binding |
| | | | | | | protein. |
| 1538 | BL00983A | 5.84 | 7.261e−10 | 12-20 | BL00983 | Ly-6/u-PAR domain |
| | | | | | | proteins. |
| 1538 | BL00983B | 8.19 | 1.643e−12 | 22-31 | BL00983 | Ly-6/u-PAR domain |
| | | | | | | proteins. |
| 1538 | BL00983C | 12.69 | 3.500e−15 | 80-95 | BL00983 | Ly-6/u-PAR domain |
| | | | | | | proteins. |
| 1539 | BL00261A | 23.97 | 5.500e−34 | 29-62 | BL00261 | Glycoprotein hormones |
| | | | | | | beta chain proteins. |
| 1539 | BL00261B | 25.64 | 6.885e−37 | 77-120 | BL00261 | Glycoprotein hormones |
| | | | | | | beta chain proteins. |
| 1539 | PR00438A | 13.54 | 1.000e−09 | 50-59 | PR00438 | GROWTH FACTOR CYSTINE |
| | | | | | | KNOT SUPERFAMILY |
| | | | | | | SIGNATURE |
| 1540 | BL00280 | 24.61 | 1.391e−37 | 132-175 | BL00280 | Pancreatic trypsin |
| | | | | | | inhibitor (Kunitz) |
| | | | | | | family proteins. |
| 1540 | BL00280 | 24.61 | 2.241e−33 | 61-104 | BL00280 | Pancreatic trypsin |
| | | | | | | inhibitor (Kunitz) |
| | | | | | | family proteins. |
| 1540 | BL00280 | 24.61 | 3.500e−29 | 224-267 | BL00280 | Pancreatic trypsin |
| | | | | | | inhibitor (Kunitz) |
| | | | | | | family proteins. |
| 1540 | PR00759A | 14.51 | 5.500e−11 | 122-136 | PR00759 | BASIC PROTEASE (KUNITZ- |
| | | | | | | TYPE) INHIBITOR FAMILY |
| | | | | | | SIGNATURE |
| 1540 | PR00759A | 14.51 | 8.579e−12 | 51-65 | PR00759 | BASIC PROTEASE (KUNITZ- |
| | | | | | | TYPE) INHIBITOR FAMILY |
| | | | | | | SIGNATURE |
| 1540 | PR00759B | 11.26 | 1.692e−11 | 79-89 | PR00759 | BASIC PROTEASE (KUNITZ- |
| | | | | | | TYPE) INHIBITOR FAMILY |
| | | | | | | SIGNATURE |
| 1540 | PR00759B | 11.26 | 2.385e−11 | 150-160 | PR00759 | BASIC PROTEASE (KUNITZ- |
| | | | | | | TYPE) INHIBITOR FAMILY |
| | | | | | | SIGNATURE |
| 1540 | PR00759B | 11.26 | 8.269e−11 | 242-252 | PR00759 | BASIC PROTEASE (KUNITZ- |
| | | | | | | TYPE) INHIBITOR FAMILY |
| | | | | | | SIGNATURE |
| 1540 | PR00759C | 14.15 | 1.000e−13 | 89-104 | PR00759 | BASIC PROTEASE (KUNITZ- |
| | | | | | | TYPE) INHIBITOR FAMILY |
| | | | | | | SIGNATURE |
| 1540 | PR00759C | 14.15 | 1.818e−15 | 160-175 | PR00759 | BASIC PROTEASE (KUNITZ- |
| | | | | | | TYPE) INHIBITOR FAMILY |
| | | | | | | SIGNATURE |
| 1540 | PR00759C | 14.15 | 3.727e−12 | 252-267 | PR00759 | BASIC PROTEASE (KUNITZ- |
| | | | | | | TYPE) INHIBITOR FAMILY |
| | | | | | | SIGNATURE |
| 1541 | BL01104A | 14.65 | 1.000e−40 | 13-51 | BL01104 | Ribosomal protein L13e |
| | | | | | | proteins. |
| 1541 | BL01104B | 15.12 | 1.000e−40 | 83-129 | BL01104 | Ribosomal protein L13e |
| | | | | | | proteins. |
| 1541 | BL01104C | 15.14 | 9.550e−28 | 174-201 | BL01104 | Ribosomal protein L13e |
| | | | | | | proteins. |
| 1543 | BL01031A | 10.55 | 4.429e−09 | 19-28 | BL01031 | Heat shock hsp20 |
| | | | | | | proteins family |
| | | | | | | profile. |
| 1543 | BL01031B | 15.78 | 5.200e−19 | 73-93 | BL01031 | Heat shock hsp20 |
| | | | | | | proteins family |
| | | | | | | profile. |
| 1543 | BL01031C | 17.68 | 8.412e−21 | 101-125 | BL01031 | Heat shock hsp20 |
| | | | | | | proteins family |
| | | | | | | profile. |
| 1543 | PR00299A | 13.53 | 8.448e−16 | 2-14 | PR00299 | ALPHA CRYSTALLIN |
| | | | | | | SIGNATURE |
| 1543 | PR00299B | 17.53 | 1.000e−16 | 19-31 | PR00299 | ALPHA CRYSTALLIN |
| | | | | | | SIGNATURE |
| 1543 | PR00299C | 8.47 | 2.317e−20 | 69-89 | PR00299 | ALPHA CRYSTALLIN |
| | | | | | | SIGNATURE |
| 1543 | PR00299D | 8.56 | 5.974e−16 | 91-104 | PR00299 | ALPHA CRYSTALLIN |
| | | | | | | SIGNATURE |
| 1543 | PR00299E | 12.26 | 6.455e−25 | 106-125 | PR00299 | ALPHA CRYSTALLIN |
| | | | | | | SIGNATURE |
| 1543 | PR00299F | 13.20 | 9.289e−21 | 128-149 | PR00299 | ALPHA CRYSTALLIN |
| | | | | | | SIGNATURE |
| 1543 | PR00299G | 5.58 | 4.825e−16 | 156-171 | PR00299 | ALPHA CRYSTALLIN |
| | | | | | | SIGNATURE |
| 1544 | BL01101A | 16.07 | 1.000e−40 | 9-53 | BL01101 | Casein kinase II |
| | | | | | | regulatory subunit |
| | | | | | | proteins. |
| 1544 | BL01101B | 10.94 | 9.000e−31 | 72-96 | BL01101 | Casein kinase II |
| | | | | | | regulatory subunit |
| | | | | | | proteins. |
| 1544 | BL01101C | 15.36 | 8.714e−37 | 97-126 | BL01101 | Casein kinase II |
| | | | | | | regulatory subunit |
| | | | | | | proteins. |
| 1544 | BL01101D | 16.87 | 1.000e−40 | 127-168 | BL01101 | Casein kinase II |
| | | | | | | regulatory subunit |
| | | | | | | proteins. |
| 1544 | PR00472A | 8.03 | 7.600e−23 | 8-24 | PR00472 | CASEIN KINASE II |
| | | | | | | REGULATORY SUBUNIT |
| | | | | | | FAMILY SIGNATURE |
| 1544 | PR00472B | 14.84 | 1.000e−19 | 25-39 | PR00472 | CASEIN KINASE II |
| | | | | | | REGULATORY SUBUNIT |
| | | | | | | FAMILY SIGNATURE |
| 1544 | PR00472C | 12.38 | 5.154e−28 | 80-101 | PR00472 | CASEIN KINASE II |
| | | | | | | REGULATORY SUBUNIT |
| | | | | | | FAMILY SIGNATURE |
| 1544 | PR00472D | 10.36 | 4.857e−28 | 105-126 | PR00472 | CASEIN KINASE II |
| | | | | | | REGULATORY SUBUNIT |
| | | | | | | FAMILY SIGNATURE |
| 1544 | PR00472E | 13.85 | 3.769e−27 | 127-148 | PR00472 | CASEIN KINASE II |
| | | | | | | REGULATORY SUBUNIT |
| | | | | | | FAMILY SIGNATURE |
| 1544 | PR00472F | 9.76 | 3.368e−23 | 155-172 | PR00472 | CASEIN KINASE II |
| | | | | | | REGULATORY SUBUNIT |
| | | | | | | FAMILY SIGNATURE |
| 1545 | BL00023 | 24.31 | 4.545e−27 | 382-418 | BL00023 | Type II fibronectin |
| | | | | | | collagen-binding domain |
| | | | | | | proteins. |
| 1545 | BL00023 | 24.31 | 8.920e−33 | 442-478 | BL00023 | Type II fibronectin |
| | | | | | | collagen-binding domain |
| | | | | | | proteins. |
| 1545 | BL01253A | 20.33 | 4.720e−09 | 589-617 | BL01253 | Type I fibronectin |
| | | | | | | domain proteins. |
| 1545 | PR00012A | 9.68 | 1.450e−14 | 221-232 | PR00012 | FIBRONECTIN TYPE I |
| | | | | | | REPEAT SIGNATURE |
| 1545 | PR00012A | 9.68 | 3.250e−14 | 2297-2308 | PR00012 | FIBRONECTIN TYPE I |
| | | | | | | REPEAT SIGNATURE |
| 1545 | PR00012A | 9.68 | 4.250e−13 | 596-607 | PR00012 | FIBRONECTIN TYPE I |
| | | | | | | REPEAT SIGNATURE |
| 1545 | PR00012A | 9.68 | 4.789e−09 | 85-96 | PR00012 | FIBRONECTIN TYPE I |
| | | | | | | REPEAT SIGNATURE |
| 1545 | PR00012A | 9.68 | 7.316e−09 | 174-185 | PR00012 | FIBRONECTIN TYPE I |
| | | | | | | REPEAT SIGNATURE |
| 1545 | PR00012A | 9.68 | 8.269e−10 | 266-277 | PR00012 | FIBRONECTIN TYPE I |
| | | | | | | REPEAT SIGNATURE |
| 1545 | PR00012B | 8.28 | 1.957e−10 | 572-580 | PR00012 | FIBRONECTIN TYPE I |
| | | | | | | REPEAT SIGNATURE |
| 1545 | PR00012B | 8.28 | 4.273e−12 | 2317-2325 | PR00012 | FIBRONECTIN TYPE I |
| | | | | | | REPEAT SIGNATURE |
| 1545 | PR00012B | 8.28 | 5.021e−10 | 105-113 | PR00012 | FIBRONECTIN TYPE I |
| | | | | | | REPEAT SIGNATURE |
| 1545 | PR00012B | 8.28 | 5.909e−12 | 525-533 | PR00012 | FIBRONECTIN TYPE I |
| | | | | | | REPEAT SIGNATURE |
| 1545 | PR00012B | 8.28 | 6.339e−09 | 196-204 | PR00012 | FIBRONECTIN TYPE I |
| | | | | | | REPEAT SIGNATURE |
| 1545 | PR00012B | 8.28 | 6.707e−11 | 2274-2282 | PR00012 | FIBRONECTIN TYPE I |
| | | | | | | REPEAT SIGNATURE |
| 1545 | PR00012B | 8.28 | 8.475e−09 | 287-295 | PR00012 | FIBRONECTIN TYPE I |
| | | | | | | REPEAT SIGNATURE |
| 1545 | PR00012B | 8.28 | 8.902e−11 | 616-624 | PR00012 | FIBRONECTIN TYPE I |
| | | | | | | REPEAT SIGNATURE |
| 1545 | PR00012B | 8.28 | 9.043e−10 | 152-160 | PR00012 | FIBRONECTIN TYPE I |
| | | | | | | REPEAT SIGNATURE |
| 1545 | PR00013A | 12.26 | 1.500e−09 | 386-395 | PR00013 | FIBRONECTIN TYPE II |
| | | | | | | REPEAT SIGNATURE |
| 1545 | PR00013A | 12.26 | 5.875e−12 | 446-455 | PR00013 | FIBRONECTIN TYPE II |
| | | | | | | REPEAT SIGNATURE |
| 1545 | PR00013B | 14.75 | 2.385e−13 | 457-469 | PR00013 | FIBRONECTIN TYPE II |
| | | | | | | REPEAT SIGNATURE |
| 1545 | PR00013C | 12.29 | 2.607e−18 | 474-489 | PR00013 | FIBRONECTIN TYPE II |
| | | | | | | REPEAT SIGNATURE |
| 1545 | PR00013C | 12.29 | 4.400e−14 | 414-429 | PR00013 | FIBRONECTIN TYPE II |
| | | | | | | REPEAT SIGNATURE |
| 1545 | PR00014A | 8.22 | 4.750e−11 | 1946-1955 | PR00014 | FIBRONECTIN TYPE III |
| | | | | | | REPEAT SIGNATURE |
| 1545 | PR00014A | 8.22 | 9.591e−09 | 1040-1049 | PR00014 | FIBRONECTIN TYPE III |
| | | | | | | REPEAT SIGNATURE |
| 1545 | PR00014B | 14.77 | 1.000e−08 | 1956-1966 | PR00014 | FIBRONECTIN TYPE III |
| | | | | | | REPEAT SIGNATURE |
| 1545 | PR00014B | 14.77 | 8.500e−09 | 864-874 | PR00014 | FIBRONECTIN TYPE III |
| | | | | | | REPEAT SIGNATURE |
| 1545 | PR00014C | 15.44 | 1.391e−09 | 797-815 | PR00014 | FIBRONECTIN TYPE III |
| | | | | | | REPEAT SIGNATURE |
| 1545 | PR00014C | 15.44 | 1.500e−11 | 2148-2166 | PR00014 | FIBRONECTIN TYPE III |
| | | | | | | REPEAT SIGNATURE |
| 1545 | PR00014C | 15.44 | 1.783e−09 | 1254-1272 | PR00014 | FIBRONECTIN TYPE III |
| | | | | | | REPEAT SIGNATURE |
| 1545 | PR00014C | 15.44 | 2.800e−10 | 688-706 | PR00014 | FIBRONECTIN TYPE III |
| | | | | | | REPEAT SIGNATURE |
| 1545 | PR00014C | 15.44 | 2.800e−12 | 1890-1908 | PR00014 | FIBRONECTIN TYPE III |
| | | | | | | REPEAT SIGNATURE |
| 1545 | PR00014C | 15.44 | 4.857e−13 | 1980-1998 | PR00014 | FIBRONECTIN TYPE III |
| | | | | | | REPEAT SIGNATURE |
| 1545 | PR00014C | 15.44 | 6.400e−10 | 1525-1543 | PR00014 | FIBRONECTIN TYPE III |
| | | | | | | REPEAT SIGNATURE |
| 1545 | PR00014C | 15.44 | 6.400e−10 | 1709-1727 | PR00014 | FIBRONECTIN TYPE III |
| | | | | | | REPEAT SIGNATURE |
| 1545 | PR00014C | 15.44 | 6.400e−10 | 887-905 | PR00014 | FIBRONECTIN TYPE III |
| | | | | | | REPEAT SIGNATURE |
| 1545 | PR00014C | 15.44 | 6.478e−09 | 1619-1637 | PR00014 | FIBRONECTIN TYPE III |
| | | | | | | REPEAT SIGNATURE |
| 1545 | PR00014C | 15.44 | 7.000e−11 | 1435-1453 | PR00014 | FIBRONECTIN TYPE III |
| | | | | | | REPEAT SIGNATURE |
| 1545 | PR00014C | 15.44 | 8.650e−10 | 1345-1363 | PR00014 | FIBRONECTIN TYPE III |
| | | | | | | REPEAT SIGNATURE |
| 1546 | BL00086 | 20.87 | 1.429e−20 | 452-483 | BL00086 | Cytochrome P450 |
| | | | | | | cysteine heme-iron |
| | | | | | | ligand proteins. |
| 1546 | PR00359I | 11.13 | 5.378e−09 | 462-473 | PR00359 | B-CLASS P450 SIGNATURE |
| 1546 | PR00385A | 14.97 | 7.882e−16 | 326-343 | PR00385 | P450 SUPERFAMILY |
| | | | | | | SIGNATURE |
| 1546 | PR00385C | 16.94 | 2.421e−10 | 379-390 | PR00385 | P450 SUPERFAMILY |
| | | | | | | SIGNATURE |
| 1546 | PR00385D | 13.11 | 5.000e−09 | 453-462 | PR00385 | P450 SUPERFAMILY |
| | | | | | | SIGNATURE |
| 1546 | PR00385E | 12.66 | 5.714e−09 | 462-473 | PR00385 | P450 SUPERFAMILY |
| | | | | | | SIGNATURE |
| 1546 | PR00408A | 14.69 | 5.680e−15 | 126-141 | PR00408 | MITOCHONDRIAL P450 |
| | | | | | | SIGNATURE |
| 1546 | PR00408B | 13.00 | 4.194e−09 | 142-152 | PR00408 | MITOCHONDRIAL P450 |
| | | | | | | SIGNATURE |
| 1546 | PR00408C | 13.23 | 5.655e−18 | 203-221 | PR00408 | MITOCHONDRIAL P450 |
| | | | | | | SIGNATURE |
| 1546 | PR00408D | 15.44 | 5.500e−21 | 326-343 | PR00408 | MITOCHONDRIAL P450 |
| | | | | | | SIGNATURE |
| 1546 | PR00408E | 9.47 | 4.545e−13 | 344-357 | PR00408 | MITOCHONDRIAL P450 |
| | | | | | | SIGNATURE |
| 1546 | PR00408F | 11.33 | 1.486e−18 | 372-390 | PR00408 | MITOCHONDRIAL P450 |
| | | | | | | SIGNATURE |
| 1546 | PR00408G | 13.17 | 9.667e−10 | 435-443 | PR00408 | MITOCHONDRIAL P450 |
| | | | | | | SIGNATURE |
| 1546 | PR00408H | 12.37 | 1.000e−12 | 453-462 | PR00408 | MITOCHONDRIAL P450 |
| | | | | | | SIGNATURE |
| 1546 | PR00408I | 8.49 | 2.125e−14 | 462-473 | PR00408 | MITOCHONDRIAL P450 |
| | | | | | | SIGNATURE |
| 1546 | PR00463G | 18.24 | 9.833e−10 | 418-442 | PR00463 | E-CLASS P450 GROUP I |
| | | | | | | SIGNATURE |
| 1546 | PR00463I | 15.02 | 8.525e−09 | 462-485 | PR00463 | E-CLASS P450 GROUP I |
| | | | | | | SIGNATURE |
| 1546 | PR00464C | 18.84 | 9.000e−11 | 315-343 | PR00464 | E-CLASS P450 GROUP II |
| | | | | | | SIGNATURE |
| 1546 | PR00464E | 18.28 | 7.511e−11 | 373-393 | PR00464 | E-CLASS P450 GROUP II |
| | | | | | | SIGNATURE |
| 1546 | PR00464I | 14.64 | 6.651e−11 | 462-485 | PR00464 | E-CLASS P450 GROUP II |
| | | | | | | SIGNATURE |
| 1546 | PR00465D | 14.64 | 6.824e−12 | 374-390 | PR00465 | E-CLASS P450 GROUP IV |
| | | | | | | SIGNATURE |
| 1546 | PR00465F | 13.37 | 6.940e−11 | 423-441 | PR00465 | E-CLASS P450 GROUP IV |
| | | | | | | SIGNATURE |
| 1548 | BL00301A | 12.41 | 3.842e−13 | 9-20 | BL00301 | GTP-binding elongation |
| | | | | | | factors proteins. |
| 1548 | BL00301B | 20.09 | 5.500e−31 | 90-121 | BL00301 | GTP-binding elongation |
| | | | | | | factors proteins. |
| 1548 | BL00301C | 11.73 | 8.200e−15 | 423-436 | BL00301 | GTP-binding elongation |
| | | | | | | factors proteins. |
| 1548 | BL00880 | 17.52 | 9.214e−09 | 99-148 | BL00880 | Acyl-CoA-binding |
| | | | | | | protein. |
| 1548 | BL01176B | 8.74 | 5.547e−12 | 87-124 | BL01176 | Initiation factor 2 |
| | | | | | | proteins. |
| 1548 | PR00315A | 11.81 | 1.818e−15 | 9-22 | PR00315 | GTP-BINDING ELONGATION |
| | | | | | | FACTOR SIGNATURE |
| 1548 | PR00315B | 11.66 | 5.800e−10 | 68-76 | PR00315 | GTP-BINDING ELONGATION |
| | | | | | | FACTOR SIGNATURE |
| 1548 | PR00315C | 13.85 | 7.750e−13 | 88-98 | PR00315 | GTP-BINDING ELONGATION |
| | | | | | | FACTOR SIGNATURE |
| 1548 | PR00315D | 10.84 | 2.895e−11 | 104-115 | PR00315 | GTP-BINDING ELONGATION |
| | | | | | | FACTOR SIGNATURE |
| 1548 | PR00315E | 12.29 | 3.864e−09 | 148-157 | PR00315 | GTP-BINDING ELONGATION |
| | | | | | | FACTOR SIGNATURE |
| 1549 | BL00514A | 11.68 | 6.885e−12 | 40-49 | BL00514 | Fibrinogen beta and |
| | | | | | | gamma chains C-terminal |
| | | | | | | domain proteins. |
| 1549 | BL00514B | 16.42 | 4.000e−15 | 150-165 | BL00514 | Fibrinogen beta and |
| | | | | | | gamma chains C-terminal |
| | | | | | | domain proteins. |
| 1549 | BL00514C | 17.41 | 1.000e−40 | 206-242 | BL00514 | Fibrinogen beta and |
| | | | | | | gamma chains C-terminal |
| | | | | | | domain proteins. |
| 1549 | BL00514D | 15.35 | 7.000e−16 | 251-263 | BL00514 | Fibrinogen beta and |
| | | | | | | gamma chains C-terminal |
| | | | | | | domain proteins. |
| 1549 | BL00514E | 14.28 | 4.000e−19 | 272-288 | BL00514 | Fibrinogen beta and |
| | | | | | | gamma chains C-terminal |
| | | | | | | domain proteins. |
| 1549 | BL00514F | 11.65 | 5.091e−15 | 304-318 | BL00514 | Fibrinogen beta and |
| | | | | | | gamma chains C-terminal |
| | | | | | | domain proteins. |
| 1549 | BL00514G | 15.98 | 1.947e−37 | 351-380 | BL00514 | Fibrinogen beta and |
| | | | | | | gamma chains C-terminal |
| | | | | | | domain proteins. |
| 1549 | BL00514H | 14.95 | 3.368e−28 | 390-414 | BL00514 | Fibrinogen beta and |
| | | | | | | gamma chains C-terminal |
| | | | | | | domain proteins. |
| 1549 | PR00171A | 10.00 | 8.875e−09 | 384-394 | PR00171 | SUGAR TRANSPORTER |
| | | | | | | SIGNATURE |
| 1551 | BL00615B | 12.25 | 3.423e−09 | 207-220 | BL00615 | C-type lectin domain |
| | | | | | | proteins. |
| 1551 | PR00770A | 13.71 | 1.231e−24 | 2-25 | PR00770 | EOSINOPHIL MAJOR BASIC |
| | | | | | | PROTEIN SIGNATURE |
| 1551 | PR00770B | 11.43 | 9.325e−17 | 88-103 | PR00770 | EOSINOPHIL MAJOR BASIC |
| | | | | | | PROTEIN SIGNATURE |
| 1551 | PR00770C | 12.19 | 5.886e−17 | 109-125 | PR00770 | EOSINOPHIL MAJOR BASIC |
| | | | | | | PROTEIN SIGNATURE |
| 1551 | PR00770D | 13.82 | 8.364e−21 | 126-142 | PR00770 | EOSINOPHIL MAJOR BASIC |
| | | | | | | PROTEIN SIGNATURE |
| 1551 | PR00770E | 8.53 | 1.000e−22 | 153-171 | PR00770 | EOSINOPHIL MAJOR BASIC |
| | | | | | | PROTEIN SIGNATURE |
| 1551 | PR00770F | 13.79 | 1.250e−26 | 172-191 | PR00770 | EOSINOPHIL MAJOR BASIC |
| | | | | | | PROTEIN SIGNATURE |
| 1551 | PR00770G | 7.65 | 3.512e−23 | 195-212 | PR00770 | EOSINOPHIL MAJOR BASIC |
| | | | | | | PROTEIN SIGNATURE |
| 1552 | BL00213A | 12.95 | 8.105e−10 | 31-44 | BL00213 | Lipocalin proteins. |
| 1552 | PR00179A | 13.78 | 5.050e−11 | 31-43 | PR00179 | LIPOCALIN SIGNATURE |
| 1552 | PR00179B | 9.56 | 5.034e−11 | 112-124 | PR00179 | LIPOCALIN SIGNATURE |
| 1552 | PR00179C | 19.02 | 8.909e−09 | 139-154 | PR00179 | LIPOCALIN SIGNATURE |
| 1553 | BL00164A | 11.58 | 1.529e−27 | 32-54 | BL00164 | Enolase proteins. |
| 1553 | BL00164B | 16.22 | 1.000e−40 | 98-140 | BL00164 | Enolase proteins. |
| 1553 | BL00164C | 15.66 | 1.000e−40 | 144-193 | BL00164 | Enolase proteins. |
| 1553 | BL00164D | 21.97 | 2.588e−38 | 220-262 | BL00164 | Enolase proteins. |
| 1553 | BL00164E | 8.80 | 9.100e−20 | 287-301 | BL00164 | Enolase proteins. |
| 1553 | BL00164F | 10.48 | 3.813e−39 | 313-348 | BL00164 | Enolase proteins. |
| 1553 | BL00164G | 12.13 | 1.000e−40 | 380-418 | BL00164 | Enolase proteins. |
| 1553 | PR00148A | 10.11 | 1.783e−18 | 35-49 | PR00148 | ENOLASE SIGNATURE |
| 1553 | PR00148B | 8.60 | 7.000e−21 | 107-123 | PR00148 | ENOLASE SIGNATURE |
| 1553 | PR00148C | 15.33 | 2.800e−16 | 164-177 | PR00148 | ENOLASE SIGNATURE |
| 1553 | PR00148D | 14.46 | 1.360e−15 | 317-328 | PR00148 | ENOLASE SIGNATURE |
| 1553 | PR00148E | 13.62 | 4.500e−17 | 340-354 | PR00148 | ENOLASE SIGNATURE |
| 1553 | PR00148F | 10.19 | 1.643e−23 | 369-386 | PR00148 | ENOLASE SIGNATURE |
| 1554 | BL00086 | 20.87 | 4.938e−24 | 438-469 | BL00086 | Cytochrome P450 |
| | | | | | | cysteine heme-iron |
| | | | | | | ligand proteins. |
| 1554 | PR00359F | 24.20 | 1.738e−09 | 382-409 | PR00359 | B-CLASS P450 SIGNATURE |
| 1554 | PR00359I | 11.13 | 3.189e−09 | 448-459 | PR00359 | B-CLASS P450 SIGNATURE |
| 1554 | PR00385A | 14.97 | 7.750e−18 | 311-328 | PR00385 | P450 SUPERFAMILY |
| | | | | | | SIGNATURE |
| 1554 | PR00385B | 10.22 | 8.650e−12 | 329-342 | PR00385 | P450 SUPERFAMILY |
| | | | | | | SIGNATURE |
| 1554 | PR00385C | 16.94 | 6.684e−10 | 364-375 | PR00385 | P450 SUPERFAMILY |
| | | | | | | SIGNATURE |
| 1554 | PR00385D | 13.11 | 1.857e−10 | 439-448 | PR00385 | P450 SUPERFAMILY |
| | | | | | | SIGNATURE |
| 1554 | PR00385E | 12.66 | 2.800e−11 | 448-459 | PR00385 | P450 SUPERFAMILY |
| | | | | | | SIGNATURE |
| 1554 | PR00408D | 15.44 | 9.211e−12 | 311-328 | PR00408 | MITOCHONDRIAL P450 |
| | | | | | | SIGNATURE |
| 1554 | PR00463A | 11.40 | 3.659e−10 | 72-91 | PR00463 | E-CLASS P450 GROUP I |
| | | | | | | SIGNATURE |
| 1554 | PR00463B | 17.50 | 9.077e−12 | 96-117 | PR00463 | E-CLASS P450 GROUP I |
| | | | | | | SIGNATURE |
| 1554 | PR00463C | 12.85 | 6.684e−18 | 187-205 | PR00463 | E-CLASS P450 GROUP I |
| | | | | | | SIGNATURE |
| 1554 | PR00463D | 14.02 | 8.875e−18 | 300-317 | PR00463 | E-CLASS P450 GROUP I |
| | | | | | | SIGNATURE |
| 1554 | PR00463E | 17.37 | 4.150e−25 | 320-346 | PR00463 | E-CLASS P450 GROUP I |
| | | | | | | SIGNATURE |
| 1554 | PR00463F | 17.63 | 1.818e−16 | 363-381 | PR00463 | E-CLASS P450 GROUP I |
| | | | | | | SIGNATURE |
| 1554 | PR00463G | 18.24 | 5.680e−19 | 404-428 | PR00463 | E-CLASS P450 GROUP I |
| | | | | | | SIGNATURE |
| 1554 | PR00463H | 12.41 | 5.154e−13 | 438-448 | PR00463 | E-CLASS P450 GROUP I |
| | | | | | | SIGNATURE |
| 1554 | PR00463I | 15.02 | 5.500e−20 | 448-471 | PR00463 | E-CLASS P450 GROUP I |
| | | | | | | SIGNATURE |
| 1554 | PR00464C | 18.84 | 4.000e−11 | 300-328 | PR00464 | E-CLASS P450 GROUP II |
| | | | | | | SIGNATURE |
| 1554 | PR00464D | 17.40 | 4.545e−12 | 329-346 | PR00464 | E-CLASS P450 GROUP II |
| | | | | | | SIGNATURE |
| 1554 | PR00464H | 13.32 | 3.423e−09 | 435-448 | PR00464 | E-CLASS P450 GROUP II |
| | | | | | | SIGNATURE |
| 1554 | PR00464I | 14.64 | 2.532e−10 | 448-471 | PR00464 | E-CLASS P450 GROUP II |
| | | | | | | SIGNATURE |
| 1554 | PR00465F | 13.37 | 3.400e−09 | 409-427 | PR00465 | E-CLASS P450 GROUP IV |
| | | | | | | SIGNATURE |
| 1555 | BL01191A | 15.57 | 1.000e−40 | 13-63 | BL01191 | Ribosomal protein S3Ae |
| | | | | | | proteins. |
| 1555 | BL01191B | 13.33 | 1.000e−40 | 89-139 | BL01191 | Ribosomal protein S3Ae |
| | | | | | | proteins. |
| 1555 | BL01191C | 16.50 | 1.000e−40 | 180-231 | BL01191 | Ribosomal protein S3Ae |
| | | | | | | proteins. |
| 1556 | BL01209 | 9.31 | 2.500e−14 | 124-136 | BL01209 | LDL-receptor class A |
| | | | | | | (LDLRA) domain |
| | | | | | | proteins. |
| 1556 | BL01209 | 9.31 | 8.342e−12 | 44-56 | BL01209 | LDL-receptor class A |
| | | | | | | (LDLRA) domain |
| | | | | | | proteins. |
| 1556 | PR00261A | 11.02 | 6.053e−17 | 115-136 | PR00261 | LOW DENSITY LIPOPROTEIN |
| | | | | | | (LDL) RECEPTOR |
| | | | | | | SIGNATURE |
| 1556 | PR00261A | 11.02 | 7.338e−12 | 35-56 | PR00261 | LOW DENSITY LIPOPROTEIN |
| | | | | | | (LDL) RECEPTOR |
| | | | | | | SIGNATURE |
| 1556 | PR00261B | 14.12 | 2.385e−15 | 115-136 | PR00261 | LOW DENSITY LIPOPROTEIN |
| | | | | | | (LDL) RECEPTOR |
| | | | | | | SIGNATURE |
| 1556 | PR00261B | 14.12 | 9.630e−12 | 35-56 | PR00261 | LOW DENSITY LIPOPROTEIN |
| | | | | | | (LDL) RECEPTOR |
| | | | | | | SIGNATURE |
| 1556 | PR00261C | 11.37 | 5.607e−09 | 78-99 | PR00261 | LOW DENSITY LIPOPROTEIN |
| | | | | | | (LDL) RECEPTOR |
| | | | | | | SIGNATURE |
| 1556 | PR00261C | 11.37 | 8.174e−13 | 35-56 | PR00261 | LOW DENSITY LIPOPROTEIN |
| | | | | | | (LDL) RECEPTOR |
| | | | | | | SIGNATURE |
| 1556 | PR00261C | 11.37 | 8.603e−17 | 115-136 | PR00261 | LOW DENSITY LIPOPROTEIN |
| | | | | | | (LDL) RECEPTOR |
| | | | | | | SIGNATURE |
| 1556 | PR00261D | 12.47 | 1.915e−16 | 115-136 | PR00261 | LOW DENSITY LIPOPROTEIN |
| | | | | | | (LDL) RECEPTOR |
| | | | | | | SIGNATURE |
| 1556 | PR00261D | 12.47 | 5.257e−11 | 35-56 | PR00261 | LOW DENSITY LIPOPROTEIN |
| | | | | | | (LDL) RECEPTOR |
| | | | | | | SIGNATURE |
| 1556 | PR00261E | 11.08 | 3.813e−13 | 115-136 | PR00261 | LOW DENSITY LIPOPROTEIN |
| | | | | | | (LDL) RECEPTOR |
| | | | | | | SIGNATURE |
| 1556 | PR00261E | 11.08 | 8.714e−11 | 35-56 | PR00261 | LOW DENSITY LIPOPROTEIN |
| | | | | | | (LDL) RECEPTOR |
| | | | | | | SIGNATURE |
| 1556 | PR00261F | 11.57 | 6.824e−12 | 115-136 | PR00261 | LOW DENSITY LIPOPROTEIN |
| | | | | | | (LDL) RECEPTOR |
| | | | | | | SIGNATURE |
| 1556 | PR00261F | 11.57 | 7.750e−12 | 35-56 | PR00261 | LOW DENSITY LIPOPROTEIN |
| | | | | | | (LDL) RECEPTOR |
| | | | | | | SIGNATURE |
| 1557 | BL00025 | 17.17 | 2.853e−17 | 89-109 | BL00025 | P-type ‘Trefoil’ domain |
| | | | | | | proteins. |
| 1557 | BL00129A | 26.21 | 1.000e−40 | 359-404 | BL00129 | Glycosyl hydrolases |
| | | | | | | family 31 proteins. |
| 1557 | BL00129B | 19.19 | 3.667e−25 | 473-499 | BL00129 | Glycosyl hydrolases |
| | | | | | | family 31 proteins. |
| 1557 | BL00129C | 15.12 | 6.211e−32 | 594-621 | BL00129 | Glycosyl hydrolases |
| | | | | | | family 31 proteins. |
| 1557 | BL00129D | 16.76 | 1.000e−40 | 632-675 | BL00129 | Glycosyl hydrolases |
| | | | | | | family 31 proteins. |
| 1557 | BL00129E | 22.60 | 1.692e−32 | 696-731 | BL00129 | Glycosyl hydrolases |
| | | | | | | family 31 proteins. |
| 1557 | BL00129F | 26.19 | 1.000e−29 | 827-864 | BL00129 | Glycosyl hydrolases |
| | | | | | | family 31 proteins. |
| 1557 | BL01103H | 11.98 | 7.840e−09 | 738-749 | BL01103 | Aspartate-semialdehyde |
| | | | | | | dehydrogenase proteins. |
| 1557 | PR00680B | 10.34 | 5.359e−10 | 99-111 | PR00680 | P-TYPE TREFOIL DOMAIN |
| | | | | | | SIGNATURE |
| 1558 | BL01033A | 16.94 | 2.929e−17 | 25-46 | BL01033 | Globins profile. |
| 1558 | BL01033B | 13.81 | 1.000e−15 | 93-104 | BL01033 | Globins profile. |
| 1558 | PR00612D | 9.76 | 2.976e−10 | 80-93 | PR00612 | ALPHA HAEMOGLOBIN |
| | | | | | | SIGNATURE |
| 1558 | PR00613B | 9.02 | 9.270e−10 | 25-48 | PR00613 | MYOGLOBIN SIGNATURE |
| 1558 | PR00613C | 10.22 | 9.630e−09 | 58-78 | PR00613 | MYOGLOBIN SIGNATURE |
| 1558 | PR00814A | 12.94 | 1.000e−22 | 30-46 | PR00814 | BETA HAEMOGLOBIN |
| | | | | | | SIGNATURE |
| 1558 | PR00814B | 9.18 | 3.571e−20 | 48-63 | PR00814 | BETA HAEMOGLOBIN |
| | | | | | | SIGNATURE |
| 1558 | PR00814C | 9.20 | 8.615e−19 | 72-89 | PR00814 | BETA HAEMOGLOBIN |
| | | | | | | SIGNATURE |
| 1558 | PR00814D | 15.25 | 2.286e−09 | 94-99 | PR00814 | BETA HEAMOGLOBIN |
| | | | | | | SIGNATURE |
| 1558 | PR00814E | 10.17 | 4.414e−14 | 128-144 | PR00814 | BETA HAEMOGLOBIN |
| | | | | | | SIGNATURE |
| 1559 | PR00014A | 8.22 | 7.955e−09 | 443-452 | PR00014 | FIBRONECTIN TYPE III |
| | | | | | | REPEAT SIGNATURE |
| 1559 | PR00014C | 15.44 | 8.826e−09 | 578-596 | PR00014 | FIBRONECTIN TYPE III |
| | | | | | | REPEAT SIGNATURE |
| 1560 | BL00222A | 11.34 | 9.438e−11 | 45-55 | BL00222 | Insulin-like growth |
| | | | | | | factor binding |
| | | | | | | proteins. |
| 1560 | BL00222B | 11.09 | 7.300e−17 | 65-80 | BL00222 | Insulin-like growth |
| | | | | | | factor binding |
| | | | | | | proteins. |
| 1560 | BL00222C | 22.97 | 1.409e−29 | 237-264 | BL00222 | Insulin-like growth |
| | | | | | | factor binding |
| | | | | | | proteins. |
| 1560 | BL00484B | 9.04 | 1.000e−17 | 238-251 | BL00484 | Thyroglobulin type-1 |
| | | | | | | repeat proteins |
| | | | | | | proteins. |
| 1560 | BL00484C | 17.01 | 5.737e−15 | 262-276 | BL00484 | Thyroglobulin type-1 |
| | | | | | | repeat proteins |
| | | | | | | proteins. |
| 1561 | BL00222B | 11.09 | 7.333e−15 | 55-70 | BL00222 | Insulin-like growth |
| | | | | | | factor binding |
| | | | | | | proteins. |
| 1561 | BL00222C | 22.97 | 2.161e−24 | 203-230 | BL00222 | Insulin-like growth |
| | | | | | | factor binding |
| | | | | | | proteins. |
| 1561 | BL00484B | 9.04 | 6.143e−16 | 204-217 | BL00484 | Thyroglobulin type-1 |
| | | | | | | repeat proteins |
| | | | | | | proteins. |
| 1561 | BL00484C | 17.01 | 1.375e−14 | 228-242 | BL00484 | Thyroglobulin type-1 |
| | | | | | | repeat proteins |
| | | | | | | proteins. |
| 1562 | BL00322A | 9.23 | 1.000e−40 | 30-81 | BL00322 | Histone H3 proteins. |
| 1562 | BL00322B | 13.68 | 1.000e−40 | 83-135 | BL00322 | Histone H3 proteins. |
| 1562 | PR00622A | 6.52 | 1.391e−19 | 3-17 | PR00622 | HISTONE H3 SIGNATURE |
| 1562 | PR00622B | 6.92 | 1.409e−19 | 17-31 | PR00622 | HISTONE H3 SIGNATURE |
| 1562 | PR00622C | 8.83 | 1.391e−28 | 34-55 | PR00622 | HISTONE H3 SIGNATURE |
| 1562 | PR00622D | 12.71 | 1.273e−22 | 58-75 | PR00622 | HISTONE H3 SIGNATURE |
| 1562 | PR00622E | 15.81 | 1.281e−21 | 80-98 | PR00622 | HISTONE H3 SIGNATURE |
| 1562 | PR00622F | 19.51 | 1.265e−22 | 98-114 | PR00622 | HISTONE H3 SIGNATURE |
| 1562 | PR00622G | 13.24 | 1.300e−28 | 114-135 | PR00622 | HISTONE H3 SIGNATURE |
| 1563 | BL00290A | 20.89 | 1.000e−14 | 221-243 | BL00290 | Immunoglobulins and |
| | | | | | | major |
| | | | | | | histocompatibility |
| | | | | | | complex proteins. |
| 1563 | BL00290A | 20.89 | 2.800e−16 | 31-53 | BL00290 | Immunoglobulins and |
| | | | | | | major |
| | | | | | | histocompatibility |
| | | | | | | complex proteins. |
| 1563 | BL00290B | 13.17 | 4.000e−21 | 278-295 | BL00290 | Immunoglobulins and |
| | | | | | | major |
| | | | | | | histocompatibility |
| | | | | | | complex proteins. |
| 1564 | BL00345B | 21.28 | 9.118e−30 | 304-354 | BL00345 | Ets-domain proteins. |
| 1564 | BL00354A | 3.83 | 8.875e−10 | 244-253 | BL00354 | HMG-I and HMG-Y DNA- |
| | | | | | | binding domain proteins |
| | | | | | | (A+T-hook). |
| 1564 | BL00354B | 3.16 | 6.468e−09 | 241-253 | BL00354 | HMG-I and HMG-Y DNA- |
| | | | | | | binding domain proteins |
| | | | | | | (A+T-hook). |
| 1564 | PR00454B | 20.78 | 8.380e−12 | 299-317 | PR00454 | ETS DOMAIN SIGNATURE |
| 1564 | PR00454C | 11.24 | 5.119e−14 | 318-336 | PR00454 | ETS DOMAIN SIGNATURE |
| 1564 | PR00454D | 10.89 | 5.860e−11 | 337-355 | PR00454 | ETS DOMAIN SIGNATURE |
| 1564 | PR00929A | 3.25 | 6.571e−09 | 244-254 | PR00929 | AT-HOOK-LIKE DOMAIN |
| | | | | | | SIGNATURE |
| 1564 | PR00929B | 4.38 | 5.018e−09 | 242-253 | PR00929 | AT-HOOK-LIKE DOMAIN |
| | | | | | | SIGNATURE |
| 1564 | PR00929C | 5.26 | 4.736e−10 | 242-252 | PR00929 | AT-HOOK-LIKE DOMAIN |
| | | | | | | SIGNATURE |
| 1564 | PR00930D | 5.16 | 9.407e−09 | 233-252 | PR00930 | HIGH MOBILITY GROUP |
| | | | | | | PROTEIN (HMGY) |
| | | | | | | SIGNATURE |
| 1565 | BL00290A | 20.89 | 7.882e−14 | 224-246 | BL00290 | Immunoglobulins and |
| | | | | | | major |
| | | | | | | histocompatibility |
| | | | | | | complex proteins. |
| 1565 | BL00290A | 20.89 | 9.526e−13 | 34-56 | BL00290 | Immunoglobulins and |
| | | | | | | major |
| | | | | | | histocompatibility |
| | | | | | | complex proteins. |
| 1565 | BL00290B | 13.17 | 6.625e−19 | 281-298 | BL00290 | Immunoglobulins and |
| | | | | | | major |
| | | | | | | histocompatibility |
| | | | | | | complex proteins. |
| 1565 | PR00818B | 6.95 | 1.614e−09 | 164-180 | PR00818 | ISLET AMYLOID PROTEIN |
| | | | | | | (AMYLIN) SIGNATURE |
| 1566 | BL00299 | 28.84 | 7.943e−26 | 97-148 | BL00299 | Ubiquitin domain |
| | | | | | | proteins. |
| 1568 | PF01105B | 25.12 | 2.016e−20 | 154-205 | PF01105 | emp24/gp25L/p24 family. |
| 1569 | BL00380F | 9.76 | 9.705e−09 | 38-48 | BL00380 | Rhodanese proteins. |
| 1569 | BL00383E | 10.35 | 1.692e−10 | 256-266 | BL00383 | Tyrosine specific |
| | | | | | | protein phosphatases |
| | | | | | | proteins. |
| 1569 | BL00415R | 7.93 | 7.655e−11 | 302-341 | BL00415 | Synapsins proteins. |
| 1569 | PR00700D | 12.47 | 4.462e−11 | 253-271 | PR00700 | PROTEIN TYROSINE |
| | | | | | | PHOSPHATASE SIGNATURE |
| 1569 | PR00716C | 17.65 | 7.750e−09 | 26-46 | PR00716 | M-PHASE INDUCER |
| | | | | | | PHOSPHATASE SIGNATURE |
| 1570 | BL00712A | 6.23 | 6.000e−25 | 2-21 | BL00712 | Ribosomal protein S17e |
| | | | | | | proteins. |
| 1570 | BL00712B | 12.56 | 1.000e−40 | 28-65 | BL00712 | Ribosomal protein S17e |
| | | | | | | proteins. |
| 1570 | BL00712C | 9.67 | 1.900e−13 | 98-107 | BL00712 | Ribosomal protein S17e |
| | | | | | | proteins. |
| 1571 | BL00388A | 23.14 | 3.483e−37 | 6-51 | BL00388 | Proteasome A-type |
| | | | | | | subunits proteins. |
| 1571 | BL00388B | 31.38 | 2.731e−29 | 62-103 | BL00388 | Proteasome A-type |
| | | | | | | subunits proteins. |
| 1571 | BL00388C | 18.79 | 2.385e−19 | 117-138 | BL00388 | Proteasome A-type |
| | | | | | | subunits proteins. |
| 1571 | BL00388D | 20.71 | 4.462e−24 | 144-174 | BL00388 | Proteasome A-type |
| | | | | | | subunits proteins. |
| 1572 | BL01002B | 7.39 | 3.118e−14 | 14-27 | BL01002 | Translationally |
| | | | | | | controlled tumor |
| | | | | | | protein. |
| 1572 | BL01002C | 21.97 | 6.143e−26 | 45-75 | BL01002 | Translationally |
| | | | | | | controlled tumor |
| | | | | | | protein. |
| 1572 | BL01002D | 18.24 | 4.706e−26 | 109-136 | BL01002 | Translationally |
| | | | | | | controlled tumor |
| | | | | | | protein. |
| 1573 | BL00298C | 16.40 | 1.000e−40 | 28-71 | BL00298 | Heat shock hsp90 |
| | | | | | | proteins family |
| | | | | | | proteins. |
| 1573 | BL00298D | 17.97 | 1.000e−40 | 84-123 | BL00298 | Heat shock hsp90 |
| | | | | | | proteins family |
| | | | | | | proteins. |
| 1573 | BL00298E | 27.30 | 1.000e−40 | 178-232 | BL00298 | Heat shock hsp90 |
| | | | | | | proteins family |
| | | | | | | proteins. |
| 1573 | BL00298F | 11.21 | 1.000e−40 | 264-318 | BL00298 | Heat shock hsp90 |
| | | | | | | proteins family |
| | | | | | | proteins. |
| 1573 | BL00298G | 24.57 | 1.000e−40 | 321-375 | BL00298 | Heat shock hsp90 |
| | | | | | | proteins family |
| | | | | | | proteins. |
| 1573 | BL00298H | 20.50 | 1.000e−40 | 409-462 | BL00298 | Heat shock hsp90 |
| | | | | | | proteins family |
| | | | | | | proteins. |
| 1573 | BL00298I | 30.07 | 1.000e−40 | 513-566 | BL00298 | Heat shock hsp90 |
| | | | | | | proteins family |
| | | | | | | proteins. |
| 1573 | BL00412D | 16.54 | 5.989e−10 | 121-171 | BL00412 | Neuromodulin (GAP-43) |
| | | | | | | proteins. |
| 1573 | PR00775D | 8.91 | 1.000e−22 | 9-26 | PR00775 | 90 KD HEAT SHOCK |
| | | | | | | PROTEIN SIGNATURE |
| 1573 | PR00775E | 8.06 | 5.500e−28 | 34-56 | PR00775 | 90 KD HEAT SHOCK |
| | | | | | | PROTEIN SIGNATURE |
| 1573 | PR00775F | 12.76 | 7.188e−21 | 85-102 | PR00775 | 90 KD HEAT SHOCK |
| | | | | | | PROTEIN SIGNATURE |
| 1573 | PR00775G | 10.64 | 1.750e−23 | 103-121 | PR00775 | 90 KD HEAT SHOCK |
| | | | | | | PROTEIN SIGNATURE |
| 1574 | BL00076A | 18.83 | 9.163e−13 | 14-43 | BL00076 | Pyridine nucleotide- |
| | | | | | | disulphide |
| | | | | | | oxidoreductases class- |
| | | | | | | I. |
| 1574 | BL00504A | 10.76 | 3.712e−10 | 15-36 | BL00504 | Fumarate reductase/ |
| | | | | | | succinate dehydrogenase |
| | | | | | | FAD-binding site |
| | | | | | | proteins. |
| 1574 | BL00623A | 12.60 | 5.500e−10 | 15-33 | BL00623 | GMC oxidoreductases |
| | | | | | | proteins. |
| 1574 | BL00836D | 22.30 | 1.574e−10 | 15-51 | BL00836 | Alanine dehydrogenase & |
| | | | | | | pyridine nucleotide |
| | | | | | | transhydrogenase. |
| 1574 | BL00977A | 20.76 | 4.051e−09 | 15-66 | BL00977 | FAD-dependent glycerol- |
| | | | | | | 3-phosphate |
| | | | | | | dehydrogenase proteins. |
| 1574 | BL00982A | 18.41 | 1.000e−13 | 17-48 | BL00982 | Bacterial-type phytoene |
| | | | | | | dehydrogenase proteins. |
| 1574 | BL01280A | 15.97 | 8.740e−09 | 15-55 | BL01280 | Glucose inhibited |
| | | | | | | division protein A |
| | | | | | | family proteins. |
| 1574 | PR00368A | 17.76 | 1.865e−10 | 15-37 | PR00368 | FAD-DEPENDENT PYRIDINE |
| | | | | | | NUCLEOTIDE REDUCTASE |
| | | | | | | SIGNATURE |
| 1574 | PR00368C | 15.74 | 3.656e−09 | 15-40 | PR00368 | FAD-DEPENDENT PYRIDINE |
| | | | | | | NUCLEOTIDE REDUCTASE |
| | | | | | | SIGNATURE |
| 1574 | PR00370A | 3.35 | 7.932e−10 | 15-30 | PR00370 | FLAVIN-CONTAINING |
| | | | | | | MONOOXYGENASE (FMO) |
| | | | | | | SIGNATURE |
| 1574 | PR00411A | 15.95 | 6.365e−10 | 15-37 | PR00411 | PYRIDINE NUCLEOTIDE |
| | | | | | | DISULPHIDE REDUCTASE |
| | | | | | | CLASS-I SIGNATURE |
| 1574 | PR00419A | 14.89 | 4.214e−09 | 15-37 | PR00419 | ADRENODOXIN REDUCTASE |
| | | | | | | FAMILY SIGNATURE |
| 1574 | PR00420A | 14.78 | 6.750e−16 | 15-37 | PR00420 | AROMATIC-RING |
| | | | | | | HYDROXYLASE |
| | | | | | | (FLAVOPROTEIN |
| | | | | | | MONOOXYGENASE) |
| | | | | | | SIGNATURE |
| 1574 | PR00469F | 16.51 | 6.241e−09 | 11-35 | PR00469 | PYRIDINE NUCLEOTIDE |
| | | | | | | DISULPHIDE REDUCTASE |
| | | | | | | CLASS-II SIGNATURE |
| 1574 | PR00757A | 6.64 | 2.324e−22 | 15-34 | PR00757 | FLAVIN-CONTAINING AMINE |
| | | | | | | OXIDASE SIGNATURE |
| 1574 | PR00757B | 12.54 | 2.286e−30 | 101-123 | PR00757 | FLAVIN-CONTAINING AMINE |
| | | | | | | OXIDASE SIGNATURE |
| 1574 | PR00757C | 13.48 | 1.600e−30 | 183-204 | PR00757 | FLAVIN-CONTAINING AMINE |
| | | | | | | OXIDASE SIGNATURE |
| 1574 | PR00757D | 6.41 | 3.571e−29 | 206-228 | PR00757 | FLAVIN-CONTAINING AMINE |
| | | | | | | OXIDASE SIGNATURE |
| 1574 | PR00757E | 11.48 | 1.000e−27 | 303-322 | PR00757 | FLAVIN-CONTAINING AMINE |
| | | | | | | OXIDASE SIGNATURE |
| 1574 | PR00757F | 15.60 | 1.129e−28 | 389-408 | PR00757 | FLAVIN-CONTAINING AMINE |
| | | | | | | OXIDASE SIGNATURE |
| 1574 | PR00757G | 4.34 | 5.345e−30 | 410-432 | PR00757 | FLAVIN-CONTAINING AMINE |
| | | | | | | OXIDASE SIGNATURE |
| 1574 | PR00757H | 9.22 | 1.096e−23 | 441-458 | PR00757 | FLAVIN-CONTAINING AMINE |
| | | | | | | OXIDASE SIGNATURE |
| 1574 | PR00757I | 12.66 | 3.368e−29 | 471-493 | PR00757 | FLAVIN-CONTAINING AMINE |
| | | | | | | OXIDASE SIGNATURE |
| 1575 | BL00290A | 20.89 | 2.421e−13 | 224-246 | BL00290 | Immunoglobulins and |
| | | | | | | major |
| | | | | | | histocompatibility |
| | | | | | | complex proteins. |
| 1575 | BL00290A | 20.89 | 4.600e−16 | 34-56 | BL00290 | Immunoglobulins and |
| | | | | | | major |
| | | | | | | histocompatibility |
| | | | | | | complex proteins. |
| 1575 | BL00290B | 13.17 | 4.000e−21 | 281-298 | BL00290 | Immunoglobulins and |
| | | | | | | major |
| | | | | | | histocompatibility |
| | | | | | | complex proteins. |
| 1576 | BL00290A | 20.89 | 6.684e−13 | 129-151 | BL00290 | Immunoglobulins and |
| | | | | | | major |
| | | | | | | histocompatibility |
| | | | | | | complex proteins. |
| 1576 | BL00290B | 13.17 | 9.308e−15 | 186-203 | BL00290 | Immunoglobulins and |
| | | | | | | major |
| | | | | | | histocompatibility |
| | | | | | | complex proteins. |
| 1577 | BL00469 | 22.22 | 1.000e−40 | 78-132 | BL00469 | Nucleoside diphosphate |
| | | | | | | kinases proteins. |
| 1578 | BL00113A | 12.74 | 4.600e−09 | 142-158 | BL00113 | Adenylate kinase |
| | | | | | | proteins. |
| 1578 | BL00291A | 4.49 | 8.448e−09 | 47-81 | BL00291 | Prion protein. |
| 1578 | BL00674B | 4.46 | 8.909e−11 | 138-159 | BL00674 | AAA-protein family |
| | | | | | | proteins. |
| 1578 | PR00300A | 9.56 | 5.442e−09 | 141-159 | PR00300 | ATP-DEPENDENT CLP |
| | | | | | | PROTEASE ATP-BINDING |
| | | | | | | SUBUNIT SIGNATURE |
| 1578 | PR00449A | 13.20 | 1.692e−10 | 139-160 | PR00449 | TRANSFORMING PROTEIN |
| | | | | | | P21 RAS SIGNATURE |
| 1578 | PR00449D | 10.79 | 6.400e−15 | 241-254 | PR00449 | TRANSFORMING PROTEIN |
| | | | | | | P21 RAS SIGNATURE |
| 1578 | PR00449E | 13.50 | 1.529e−11 | 276-298 | PR00449 | TRANSFORMING PROTEIN |
| | | | | | | P21 RAS SIGNATURE |
| 1578 | PR00830A | 8.41 | 8.644e−09 | 145-164 | PR00830 | ENDOPEPTIDASE LA (LON) |
| | | | | | | SERINE PROTEASE (S16) |
| | | | | | | SIGNATURE |
| 1579 | BL01257A | 11.15 | 1.000e−40 | 2-44 | BL01257 | Ribosomal protein L10e |
| | | | | | | proteins. |
| 1579 | BL01257B | 12.56 | 1.000e−40 | 62-101 | BL01257 | Ribosomal protein L10e |
| | | | | | | proteins. |
| 1579 | BL01257C | 11.43 | 1.000e−40 | 102-136 | BL01257 | Ribosomal protein L10e |
| | | | | | | proteins. |
| 1579 | BL01257D | 18.80 | 1.000e−40 | 137-176 | BL01257 | Ribosomal protein L10e |
| | | | | | | proteins. |
| 1579 | PF00774D | 10.59 | 9.250e−10 | 189-214 | PF00774 | Dihydropyridine |
| | | | | | | sensitive L-type |
| | | | | | | calcium channel (Beta |
| | | | | | | subuni. |
| 1580 | BL01204A | 17.74 | 1.000e−40 | 19-66 | BL01204 | NF-kappa-B/Rel/dorsal |
| | | | | | | domain proteins. |
| 1580 | BL01204B | 15.41 | 4.000e−16 | 113-126 | BL01204 | NF-kappa-B/Rel/dorsal |
| | | | | | | domain proteins. |
| 1580 | BL01204C | 13.93 | 9.727e−18 | 150-168 | BL01204 | NF-kappa-B/Rel/dorsal |
| | | | | | | domain proteins. |
| 1580 | BL01204D | 16.42 | 1.000e−40 | 185-228 | BL01204 | NF-kappa-B/Rel/dorsal |
| | | | | | | domain proteins. |
| 1580 | BL01204E | 13.83 | 8.875e−31 | 233-257 | BL01204 | NF-kappa-B/Rel/dorsal |
| | | | | | | domain proteins. |
| 1580 | PR00057A | 10.14 | 1.450e−24 | 25-42 | PR00057 | TRANSCRIPTION FACTOR |
| | | | | | | NF-KB SIGNATURE |
| 1580 | PR00057B | 11.81 | 5.629e−15 | 175-189 | PR00057 | TRANSCRIPTION FACTOR |
| | | | | | | NF-KB SIGNATURE |
| 1580 | PR00057C | 7.43 | 4.462e−25 | 208-228 | PR00057 | TRANSCRIPTION FACTOR |
| | | | | | | NF-KB SIGNATURE |
| 1580 | PR00057D | 11.94 | 1.563e−24 | 239-257 | PR00057 | TRANSCRIPTION FACTOR |
| | | | | | | NF-KB SIGNATURE |
| 1580 | PR00057E | 15.74 | 4.000e−14 | 280-294 | PR00057 | TRANSCRIPTION FACTOR |
| | | | | | | NF-KB SIGNATURE |
| 1581 | BL00284A | 15.64 | 7.231e−17 | 54-77 | BL00284 | Serpins proteins. |
| 1581 | BL00284B | 17.99 | 1.783e−15 | 155-175 | BL00284 | Serpins proteins. |
| 1581 | BL00284C | 28.56 | 4.913e−31 | 184-225 | BL00284 | Serpins proteins. |
| 1581 | BL00284D | 16.34 | 6.538e−18 | 296-322 | BL00284 | Serpins proteins. |
| 1581 | BL00284E | 19.15 | 8.875e−23 | 378-402 | BL00284 | Serpins proteins. |
| 1582 | BL01251A | 17.37 | 4.240e−31 | 1-36 | BL01251 | Proliferating cell |
| | | | | | | nuclear antigen |
| | | | | | | proteins. |
| 1582 | BL01251B | 17.25 | 1.000e−40 | 37-82 | BL01251 | Proliferating cell |
| | | | | | | nuclear antigen |
| | | | | | | proteins. |
| 1582 | BL01251C | 19.35 | 1.000e−40 | 108-157 | BL01251 | Proliferating cell |
| | | | | | | nuclear antigen |
| | | | | | | proteins. |
| 1582 | BL01251D | 23.72 | 1.000e−40 | 197-250 | BL01251 | Proliferating cell |
| | | | | | | nuclear antigen |
| | | | | | | proteins. |
| 1582 | PR00339A | 5.83 | 6.400e−21 | 10-29 | PR00339 | PROLIFERATING CELL |
| | | | | | | NUCLEAR ANTIGEN |
| | | | | | | (CYCLIN) SIGNATURE |
| 1582 | PR00339B | 9.94 | 3.250e−20 | 34-52 | PR00339 | PROLIFERATING CELL |
| | | | | | | NUCLEAR ANTIGEN |
| | | | | | | (CYCLIN) SIGNATURE |
| 1582 | PR00339C | 13.91 | 7.158e−29 | 56-80 | PR00339 | PROLIFERATING CELL |
| | | | | | | NUCLEAR ANTIGEN |
| | | | | | | (CYCLIN) SIGNATURE |
| 1582 | PR00339D | 12.90 | 8.650e−26 | 110-132 | PR00339 | PROLIFERATING CELL |
| | | | | | | NUCLEAR ANTIGEN |
| | | | | | | (CYCLIN) SIGNATURE |
| 1582 | PR00339E | 14.05 | 2.000e−18 | 203-217 | PR00339 | PROLIFERATING CELL |
| | | | | | | NUCLEAR ANTIGEN |
| | | | | | | (CYCLIN) SIGNATURE |
| 1582 | PR00339F | 10.56 | 1.000e−22 | 241-258 | PR00339 | PROLIFERATING CELL |
| | | | | | | NUCLEAR ANTIGEN |
| | | | | | | (CYCLIN) SIGNATURE |
| 1583 | BL00507A | 8.79 | 4.643e−09 | 309-329 | BL00507 | Nickel-dependent |
| | | | | | | hydrogenases large |
| | | | | | | subunit proteins. |
| 1583 | PD02870A | 14.10 | 1.563e−17 | 39-55 | PD02870 | RECEPTOR INTERLEUKIN-1 |
| | | | | | | PRECURSOR. |
| 1583 | PD02870B | 18.83 | 7.968e−34 | 93-125 | PD02870 | RECEPTOR INTERLEUKIN-1 |
| | | | | | | PRECURSOR. |
| 1583 | PD02870C | 24.41 | 9.419e−31 | 136-170 | PD02870 | RECEPTOR INTERLEUKIN-1 |
| | | | | | | PRECURSOR. |
| 1583 | PD02870D | 15.74 | 4.000e−33 | 192-226 | PD02870 | RECEPTOR INTERLEUKIN-1 |
| | | | | | | PRECURSOR. |
| 1583 | PD02870E | 12.75 | 1.545e−16 | 244-260 | PD02870 | RECEPTOR INTERLEUKIN-1 |
| | | | | | | PRECURSOR. |
| 1583 | PD02870F | 22.82 | 4.536e−22 | 301-325 | PD02870 | RECEPTOR INTERLEUKIN-1 |
| | | | | | | PRECURSOR. |
| 1584 | BL01024A | 10.26 | 1.000e−40 | 26-72 | BL01024 | Protein phosphatase 2A |
| | | | | | | regulatory subunit PR55 |
| | | | | | | proteins. |
| 1584 | BL01024B | 8.91 | 1.000e−40 | 90-130 | BL01024 | Protein phosphatase 2A |
| | | | | | | regulatory subunit PR55 |
| | | | | | | proteins. |
| 1584 | BL01024C | 7.80 | 1.000e−40 | 150-188 | BL01024 | Protein phosphatase 2A |
| | | | | | | regulatory subunit PR55 |
| | | | | | | proteins. |
| 1584 | BL01024D | 13.22 | 1.000e−40 | 189-225 | BL01024 | Protein phosphatase 2A |
| | | | | | | regulatory subunit PR55 |
| | | | | | | proteins. |
| 1584 | BL01024E | 11.96 | 1.000e−40 | 226-269 | BL01024 | Protein phosphatase 2A |
| | | | | | | regulatory subunit PR55 |
| | | | | | | proteins. |
| 1584 | BL01024F | 9.42 | 1.000e−40 | 270-320 | BL01024 | Protein phosphatase 2A |
| | | | | | | regulatory subunit PR55 |
| | | | | | | proteins. |
| 1584 | BL01024G | 11.09 | 1.000e−40 | 321-352 | BL01024 | Protein phosphatase 2A |
| | | | | | | regulatory subunit PR55 |
| | | | | | | proteins. |
| 1584 | BL01024H | 13.88 | 1.000e−40 | 393-445 | BL01024 | Protein phosphatase 2A |
| | | | | | | regulatory subunit PR55 |
| | | | | | | proteins. |
| 1584 | PR00600A | 11.61 | 6.000e−26 | 35-55 | PR00600 | PROTEIN PHOSPHATASE |
| | | | | | | PP2A 55 kd REGULATORY |
| | | | | | | SUBUNIT SIGNATURE |
| 1584 | PR00600B | 11.27 | 1.500e−37 | 70-98 | PR00600 | PROTEIN PHOSPHATASE |
| | | | | | | PP2A 55 kd REGULATORY |
| | | | | | | SUBUNIT SIGNATURE |
| 1584 | PR00600C | 10.70 | 3.842e−35 | 99-127 | PR00600 | PROTEIN PHOSPHATASE |
| | | | | | | PP2A 55 kd REGULATORY |
| | | | | | | SUBUNIT SIGNATURE |
| 1584 | PR00500D | 10.70 | 4.667e−36 | 176-203 | PR00600 | PROTEIN PHOSPHATASE |
| | | | | | | PP2A 55 kd REGULATORY |
| | | | | | | SUBUNIT SIGNATURE |
| 1584 | PR00600E | 12.87 | 1.000e−34 | 204-231 | PR00600 | PROTEIN PHOSPHATASE |
| | | | | | | PP2A 55 kd REGULATORY |
| | | | | | | SUBUNIT SIGNATURE |
| 1584 | PR00600F | 18.73 | 9.625e−36 | 232-260 | PR00600 | PROTEIN PHOSPHATASE |
| | | | | | | PP2A 55 kd REGULATORY |
| | | | | | | SUBUNIT SIGNATURE |
| 1584 | PR00600G | 8.77 | 6.143e−35 | 261-288 | PR00600 | PROTEIN PHOSPHATASE |
| | | | | | | PP2A 55 kd REGULATORY |
| | | | | | | SUBUNIT SIGNATURE |
| 1584 | PR00600H | 8.99 | 5.500e−36 | 289-316 | PR00600 | PROTEIN PHOSPHATASE |
| | | | | | | PP2A 55 kd REGULATORY |
| | | | | | | SUBUNIT SIGNATURE |
| 1584 | PR00600I | 10.13 | 6.625e−34 | 317-342 | PR00600 | PROTEIN PHOSPHATASE |
| | | | | | | PP2A 55 kd REGULATORY |
| | | | | | | SUBUNIT SIGNATURE |
| 1584 | PR00600J | 12.56 | 7.000e−37 | 343-369 | PR00600 | PROTEIN PHOSPHATASE |
| | | | | | | PP2A 55 kd REGULATORY |
| | | | | | | SUBUNIT SIGNATURE |
| 1584 | PR00600K | 11.12 | 3.647e−37 | 413-442 | PR00600 | PROTEIN PHOSPHATASE |
| | | | | | | PP2A 55 kd REGULATORY |
| | | | | | | SUBUNIT SIGNATURE |
| 1585 | DM00372A | 19.18 | 1.000e−40 | 19-63 | DM00372 | CARCINOEMBRYONIC |
| | | | | | | ANTIGEN PRECURSOR |
| | | | | | | AMINO-TERMINAL DOMAIN. |
| 1585 | DM00372B | 20.31 | 9.763e−35 | 82-126 | DM00372 | CARCINOEMBRYONIC |
| | | | | | | ANTIGEN PRECURSOR |
| | | | | | | AMINO-TERMINAL DOMAIN. |
| 1585 | DM00372C | 23.69 | 6.921e−23 | 321-356 | DM00372 | CARCINOEMBRYONIC |
| | | | | | | ANTIGEN PRECURSOR |
| | | | | | | AMINO-TERMINAL DOMAIN. |
| 1586 | BL00214A | 21.17 | 1.257e−18 | 5-30 | BL00214 | Cytosolic fatty-acid |
| | | | | | | binding proteins. |
| 1586 | BL00214B | 26.51 | 6.516e−26 | 46-90 | BL00214 | Cytosolic fatty-acid |
| | | | | | | binding proteins. |
| 1586 | PR00178A | 15.07 | 5.846e−17 | 6-26 | PR00178 | FATTY ACID-BINDING |
| | | | | | | PROTEIN SIGNATURE |
| 1586 | PR00178B | 10.52 | 5.800e−10 | 40-51 | PR00178 | FATTY ACID-BINDING |
| | | | | | | PROTEIN SIGNATURE |
| 1586 | PR00178C | 20.54 | 3.919e−19 | 66-93 | PR00178 | FATTY ACID-BINDING |
| | | | | | | PROTEIN SIGNATURE |
| 1586 | PR00178D | 13.52 | 3.132e−14 | 112-130 | PR00178 | FATTY ACID-BINDING |
| | | | | | | PROTEIN SIGNATURE |
| 1587 | BL00107A | 18.39 | 3.842e−18 | 768-798 | BL00107 | Protein kinases ATP- |
| | | | | | | binding region |
| | | | | | | proteins. |
| 1587 | BL00107B | 13.31 | 9.100e−14 | 835-850 | BL00107 | Protein kinases ATP- |
| | | | | | | binding region |
| | | | | | | proteins. |
| 1587 | BL00239B | 25.15 | 2.849e−14 | 634-681 | BL00239 | Receptor tyrosine |
| | | | | | | kinase class II |
| | | | | | | proteins. |
| 1587 | BL00239C | 18.75 | 3.684e−13 | 755-777 | BL00239 | Receptor tyrosine |
| | | | | | | kinase class II |
| | | | | | | proteins. |
| 1587 | BL00239D | 16.81 | 4.356e−11 | 780-805 | BL00239 | Receptor tyrosine |
| | | | | | | kinase class II |
| | | | | | | proteins. |
| 1587 | BL00239E | 17.14 | 6.891e−32 | 807-856 | BL00239 | Receptor tyrosine |
| | | | | | | kinase class II |
| | | | | | | proteins. |
| 1587 | BL00239F | 28.15 | 1.632e−19 | 862-906 | BL00239 | Receptor tyrosine |
| | | | | | | kinase class II |
| | | | | | | proteins. |
| 1587 | BL00240A | 22.72 | 6.516e−20 | 21-49 | BL00240 | Receptor tyrosine |
| | | | | | | kinase class III |
| | | | | | | proteins. |
| 1587 | BL00240B | 24.70 | 9.500e−20 | 417-440 | BL00240 | Receptor tyrosine |
| | | | | | | kinase class III |
| | | | | | | proteins. |
| 1587 | BL00240C | 22.58 | 1.000e−40 | 574-622 | BL00240 | Receptor tyrosine |
| | | | | | | kinase class III |
| | | | | | | proteins. |
| 1587 | BL00240D | 23.07 | 1.000e−40 | 623-677 | BL00240 | Receptor tyrosine |
| | | | | | | kinase class III |
| | | | | | | proteins. |
| 1587 | BL00240E | 11.56 | 1.000e−40 | 754-791 | BL00240 | Receptor tyrosine |
| | | | | | | kinase class III |
| | | | | | | proteins. |
| 1587 | BL00240F | 17.74 | 1.000e−40 | 806-853 | BL00240 | Receptor tyrosine |
| | | | | | | kinase class III |
| | | | | | | proteins. |
| 1587 | BL00240G | 28.45 | 1.000e−40 | 854-906 | BL00240 | Receptor tyrosine |
| | | | | | | kinase class III |
| | | | | | | proteins. |
| 1587 | BL00790M | 8.74 | 5.747e−09 | 759-780 | BL00790 | Receptor tyrosine |
| | | | | | | kinase class V |
| | | | | | | proteins. |
| 1587 | BL00790N | 13.25 | 1.098e-11 | 781-807 | BL00790 | Receptor tyrosine |
| | | | | | | kinase class V |
| | | | | | | proteins. |
| 1587 | BL00790O | 7.68 | 3.302e-15 | 814-846 | BL00790 | Receptor tyrosine |
| | | | | | | kinase class V |
| | | | | | | proteins. |
| 1587 | BL00790Q | 15.61 | 3.536e-10 | 873-921 | BL00790 | Receptor tyrosine |
| | | | | | | kinase class V |
| | | | | | | proteins. |
| 1587 | BL50001B | 17.40 | 4.130e-10 | 765-785 | BL50001 | Src homology 2 (SH2) |
| | | | | | | domain proteins |
| | | | | | | profile. |
| 1587 | BL50001D | 11.00 | 4.750e−09 | 835-845 | BL50001 | Src homology 2 (SH2) |
| | | | | | | domain proteins |
| | | | | | | profile. |
| 1587 | PR00109A | 15.00 | 8.615e−15 | 663-676 | PR00109 | TYROSINE KINASE |
| | | | | | | CATALYTIC DOMAIN |
| | | | | | | SIGNATURE |
| 1587 | PR00109B | 12.27 | 5.500e−18 | 768-786 | PR00109 | TYROSINE KINASE |
| | | | | | | CATALYTIC DOMAIN |
| | | | | | | SIGNATURE |
| 1587 | PR00109C | 12.85 | 2.500e−14 | 817-827 | PR00109 | TYROSINE KINASE |
| | | | | | | CATALYTIC DOMAIN |
| | | | | | | SIGNATURE |
| 1587 | PR00109D | 17.04 | 7.158e−23 | 836-858 | PR00109 | TYROSINE KINASE |
| | | | | | | CATALYTIC DOMAIN |
| | | | | | | SIGNATURE |
| 1587 | PR00109E | 14.41 | 1.321e−16 | 881-903 | PR00109 | TYROSINE KINASE |
| | | | | | | CATALYTIC DOMAIN |
| | | | | | | SIGNATURE |
| 1590 | BL00442B | 14.98 | 5.765e−09 | 237-248 | BL00442 | Glutamine |
| | | | | | | amidotransferases |
| | | | | | | class-I proteins. |
| 1591 | BL00962A | 11.87 | 1.600e−10 | 13-25 | BL00962 | Ribosomal protein S2 |
| | | | | | | proteins. |
| 1591 | BL00962B | 36.15 | 4.600e−36 | 28-81 | BL00962 | Ribosomal protein S2 |
| | | | | | | proteins. |
| 1591 | BL00962C | 15.90 | 9.591e−17 | 94-111 | BL00962 | Ribosomal protein S2 |
| | | | | | | proteins. |
| 1591 | BL00962D | 22.51 | 5.500e−35 | 119-162 | BL00962 | Ribosomal protein S2 |
| | | | | | | proteins. |
| 1591 | PR00395A | 16.93 | 6.464e−15 | 15-33 | PR00395 | RIBOSOMAL PROTEIN S2 |
| | | | | | | SIGNATURE |
| 1591 | PR00395B | 10.55 | 1.771e−10 | 45-54 | PR00395 | RIBOSOMAL PROTEIN S2 |
| | | | | | | SIGNATURE |
| 1591 | PR00395C | 16.17 | 1.000e−17 | 94-111 | PR00395 | RIBOSOMAL PROTEIN S2 |
| | | | | | | SIGNATURE |
| 1591 | PR00395D | 13.04 | 7.000e−17 | 119-136 | PR00395 | RIBOSOMAL PROTEIN S2 |
| | | | | | | SIGNATURE |
| 1591 | PR00395E | 14.46 | 4.103e−11 | 136-147 | PR00395 | RIBOSOMAL PROTEIN S2 |
| | | | | | | SIGNATURE |
| 1591 | PR00395F | 10.56 | 6.400e−16 | 157-171 | PR00395 | RIBOSOMAL PROTEIN S2 |
| | | | | | | SIGNATURE |
| 1592 | BL00740B | 19.76 | 3.813e−09 | 690-710 | BL00740 | MAM domain proteins. |
| 1592 | PR00597A | 12.96 | 1.000e−25 | 375-396 | PR00597 | GELSOLIN FAMILY |
| | | | | | | SIGNATURE |
| 1592 | PR00597A | 12.96 | 4.575e−09 | 742-763 | PR00597 | GELSOLIN FAMILY |
| | | | | | | SIGNATURE |
| 1592 | PR00597B | 9.78 | 5.629e−09 | 85-101 | PR00597 | GELSOLIN FAMILY |
| | | | | | | SIGNATURE |
| 1592 | PR00597B | 9.78 | 7.000e−20 | 464-480 | PR00597 | GELSOLIN FAMILY |
| | | | | | | SIGNATURE |
| 1592 | PR00597C | 14.19 | 9.000e−23 | 489-507 | PR00597 | GELSOLIN FAMILY |
| | | | | | | SIGNATURE |
| 1592 | PR00597D | 12.77 | 4.522e−24 | 522-542 | PR00597 | GELSOLIN FAMILY |
| | | | | | | SIGNATURE |
| 1592 | PR00597D | 12.77 | 7.723e−09 | 144-164 | PR00597 | GELSOLIN FAMILY |
| | | | | | | SIGNATURE |
| 1592 | PR00597E | 13.46 | 4.130e−21 | 576-596 | PR00597 | GELSOLIN FAMILY |
| | | | | | | SIGNATURE |
| 1592 | PR00597F | 16.29 | 4.522e−21 | 634-653 | PR00597 | GELSOLIN FAMILY |
| | | | | | | SIGNATURE |
| 1592 | PR00597G | 8.55 | 1.429e−28 | 690-712 | PR00597 | GELSOLIN FAMILY |
| | | | | | | SIGNATURE |
| 1592 | PR00597H | 15.32 | 5.500e−22 | 719-738 | PR00597 | GELSOLIN FAMILY |
| | | | | | | SIGNATURE |
| 1595 | BL01019A | 13.20 | 3.629e−09 | 46-85 | BL01019 | ADP-ribosylation |
| | | | | | | factors family |
| | | | | | | proteins. |
| 1595 | BL01115A | 10.22 | 2.688e−14 | 12-55 | BL01115 | GTP-binding nuclear |
| | | | | | | protein ran proteins. |
| 1595 | PR00449A | 13.20 | 7.000e−20 | 12-33 | PR00449 | TRANSFORMING PROTEIN |
| | | | | | | P21 RAS SIGNATURE |
| 1595 | PR00449B | 14.34 | 7.500e−12 | 35-51 | PR00449 | TRANSFORMING PROTEIN |
| | | | | | | P21 RAS SIGNATURE |
| 1595 | PR00449C | 17.27 | 5.500e−24 | 53-75 | PR00449 | TRANSFORMING PROTEIN |
| | | | | | | P21 RAS SIGNATURE |
| 1595 | PR00449D | 10.79 | 5.500e−14 | 115-128 | PR00449 | TRANSFORMING PROTEIN |
| | | | | | | P21 RAS SIGNATURE |
| 1595 | PR00449E | 13.50 | 5.263e−17 | 150-172 | PR00449 | TRANSFORMING PROTEIN |
| | | | | | | P21 RAS SIGNATURE |
| 1596 | BL00578A | 14.24 | 2.800e−37 | 1-31 | BL00578 | Ribosomal protein S6e |
| | | | | | | proteins. |
| 1596 | BL00578B | 15.15 | 1.000e−40 | 39-89 | BL00578 | Ribosomal protein S6e |
| | | | | | | proteins. |
| 1596 | BL00578C | 9.53 | 1.000e−40 | 120-157 | BL00578 | Ribosomal protein S6e |
| | | | | | | proteins. |
| 1596 | BL00578D | 16.68 | 1.000e−40 | 164-213 | BL00578 | Ribosomal protein S6e |
| | | | | | | proteins. |
| 1597 | BL00472B | 14.67 | 8.448e−12 | 31-48 | BL00472 | Small cytokines |
| | | | | | | (intercrine/chemokine) |
| | | | | | | C-C subfamily signatur. |
| 1597 | PR00436D | 12.23 | 1.106e−10 | 72-95 | PR00436 | INTERLEUKIN-8 SIGNATURE |
| 1598 | DM00895E | 15.72 | 3.333e−11 | 527-551 | DM00895 | 7 kw REVERSE |
| | | | | | | TRANSCRIPTASE RNA |
| | | | | | | POLYMERASE. |
| 1598 | DM01354A | 11.77 | 4.462e−29 | 13-40 | DM01354 | kw TRANSCRIPTASE |
| | | | | | | REVERSE II ORF2. |
| 1598 | DM01354B | 14.71 | 9.280e−33 | 41-68 | DM01354 | kw TRANSCRIPTASE |
| | | | | | | REVERSE II ORF2. |
| 1598 | DM01354C | 8.79 | 5.680e−21 | 69-86 | DM01354 | kw TRANSCRIPTASE |
| | | | | | | REVERSE II ORF2. |
| 1598 | DM01354D | 10.59 | 4.960e−32 | 87-114 | DM01354 | kw TRANSCRIPTASE |
| | | | | | | REVERSE II ORF2. |
| 1598 | DM01354E | 18.69 | 3.025e−28 | 115-144 | DM01354 | kw TRANSCRIPTASE |
| | | | | | | REVERSE II ORF2. |
| 1598 | DM01354F | 14.56 | 7.943e−37 | 145-180 | DM01354 | kw TRANSCRIPTASE |
| | | | | | | REVERSE II ORF2. |
| 1598 | DM01354G | 11.57 | 9.500e−37 | 181-211 | DM01354 | kw TRANSCRIPTASE |
| | | | | | | REVERSE II ORF2. |
| 1598 | DM01354H | 18.00 | 8.071e−30 | 212-251 | DM01354 | kw TRANSCRIPTASE |
| | | | | | | REVERSE II ORF2. |
| 1598 | DM01354I | 15.55 | 2.800e−39 | 252-292 | DM01354 | kw TRANSCRIPTASE |
| | | | | | | REVERSE II ORF2. |
| 1598 | DM01354J | 15.12 | 8.541e−31 | 293-324 | DM01354 | kw TRANSCRIPTASE |
| | | | | | | REVERSE II ORF2. |
| 1598 | DM01354K | 9.35 | 1.200e−30 | 325-356 | DM01354 | kw TRANSCRIPTASE |
| | | | | | | REVERSE II ORF2. |
| 1598 | DM01354L | 11.73 | 4.000e−30 | 357-383 | DM01354 | kw TRANSCRIPTASE |
| | | | | | | REVERSE II ORF2. |
| 1598 | DM01354M | 12.50 | 4.960e−33 | 384-413 | DM01354 | kw TRANSCRIPTASE |
| | | | | | | REVERSE II ORF2. |
| 1598 | DM01354N | 13.17 | 1.000e−40 | 414-458 | DM01354 | kw TRANSCRIPTASE |
| | | | | | | REVERSE II ORF2. |
| 1598 | DM01354O | 8.73 | 1.000e−40 | 465-510 | DM01354 | kw TRANSCRIPTASE |
| | | | | | | REVERSE II ORF2. |
| 1598 | DM01354P | 9.18 | 3.308e−40 | 511-544 | DM01354 | kw TRANSCRIPTASE |
| | | | | | | REVERSE II ORF2. |
| 1598 | DM01354Q | 9.23 | 1.000e−40 | 545-599 | DM01354 | kw TRANSCRIPTASE |
| | | | | | | REVERSE II ORF2. |
| 1598 | DM01354R | 8.50 | 5.714e−31 | 600-629 | DM01354 | kw TRANSCRIPTASE |
| | | | | | | REVERSE II ORF2. |
| 1598 | DM01354S | 11.61 | 7.300e−26 | 630-650 | DM01354 | kw TRANSCRIPTASE |
| | | | | | | REVERSE II ORF2. |
| 1598 | DM01354T | 9.48 | 1.000e−40 | 651-693 | DM01354 | kw TRANSCRIPTASE |
| | | | | | | REVERSE II ORF2. |
| 1598 | DM01354U | 12.24 | 7.171e−21 | 740-759 | DM01354 | kw TRANSCRIPTASE |
| | | | | | | REVERSE II ORF2. |
| 1598 | DM01354V | 12.97 | 1.000e−40 | 760-806 | DM01354 | kw TRANSCRIPTASE |
| | | | | | | REVERSE II ORF2. |
| 1598 | DM01354W | 12.64 | 6.760e−23 | 807-826 | DM01354 | kw TRANSCRIPTASE |
| | | | | | | REVERSE II ORF2. |
| 1598 | DM01354X | 13.86 | 1.000e−40 | 827-865 | DM01354 | kw TRANSCRIPTASE |
| | | | | | | REVERSE II ORF2. |
| 1598 | DM01354Y | 10.69 | 1.000e−40 | 876-915 | DM01354 | kw TRANSCRIPTASE |
| | | | | | | REVERSE II ORF2. |
| 1598 | DM01354Z | 9.06 | 1.000e−40 | 1190-1238 | DM01354 | kw TRANSCRIPTASE |
| | | | | | | REVERSE II ORF2. |
| 1600 | BL00322A | 9.23 | 1.000e−40 | 32-83 | BL00322 | Histone H3 proteins. |
| 1600 | BL00322B | 13.68 | 1.000e−40 | 85-137 | BL00322 | Histone H3 proteins. |
| 1600 | PR00622A | 6.52 | 1.391e−19 | 5-19 | PR00622 | HISTONE H3 SIGNATURE |
| 1600 | PR00622B | 6.92 | 1.409e−19 | 19-33 | PR00622 | HISTONE H3 SIGNATURE |
| 1600 | PR00622C | 8.83 | 1.391e−28 | 36-57 | PR00622 | HISTONE H3 SIGNATURE |
| 1600 | PR00622D | 12.71 | 1.273e−22 | 60-77 | PR00622 | HISTONE H3 SIGNATURE |
| 1600 | PR00622E | 15.81 | 1.281e−21 | 82-100 | PR00622 | HISTONE H3 SIGNATURE |
| 1600 | PR00622F | 19.51 | 1.265e−22 | 100-116 | PR00622 | HISTONE H3 SIGNATURE |
| 1600 | PR00622G | 13.24 | 1.300e−28 | 116-137 | PR00622 | HISTONE H3 SIGNATURE |
| 1601 | BL00065A | 31.92 | 4.682e−27 | 115-156 | BL00065 | D-isomer specific 2- |
| | | | | | | hydroxyacid |
| | | | | | | dehydrogenases NAD- |
| | | | | | | binding si. |
| 1601 | BL00065B | 16.97 | 7.652e−19 | 179-203 | BL00065 | D-isomer specific 2- |
| | | | | | | hydroxyacid |
| | | | | | | dehydrogenases NAD- |
| | | | | | | binding si. |
| 1601 | BL00065C | 27.25 | 6.000e−37 | 253-303 | BL00065 | D-isomer specific 2- |
| | | | | | | hydroxyacid |
| | | | | | | dehydrogenases NAD- |
| | | | | | | binding si. |
| 1601 | BL00071A | 5.81 | 8.258e−09 | 184-195 | BL00071 | Glyceraldehyde 3- |
| | | | | | | phosphate dehydrogenase |
| | | | | | | proteins. |
| 1603 | BL00388A | 23.14 | 5.500e−39 | 8-53 | BL00388 | Proteasome A-type |
| | | | | | | subunits proteins. |
| 1603 | BL00388B | 31.38 | 7.207e−27 | 66-107 | BL00388 | Proteasome A-type |
| | | | | | | subunits proteins. |
| 1603 | BL00388C | 18.79 | 4.706e−16 | 121-142 | BL00388 | Proteasome A-type |
| | | | | | | subunits proteins. |
| 1603 | BL00388D | 20.71 | 6.897e−23 | 149-179 | 8L00388 | Proteasome A-type |
| | | | | | | subunits proteins. |
| 1603 | PF00227 | 14.68 | 6.294e−10 | 15-26 | PF00227 | Proteasome A-type and |
| | | | | | | B-type. |
| 1604 | BL00053A | 8.83 | 5.320e−12 | 5-17 | BL00053 | Ribosomal protein S8 |
| | | | | | | proteins. |
| 1604 | BL00053B | 14.56 | 4.789e−14 | 58-75 | BL00053 | Ribosomal protein S8 |
| | | | | | | proteins. |
| 1604 | BL00053C | 16.71 | 5.500e−26 | 98-130 | BL00053 | Ribosomal protein S8 |
| | | | | | | proteins. |
| 1607 | BL01170A | 12.34 | 9.143e−40 | 139-174 | BL01170 | Ribosomal protein L6e |
| | | | | | | proteins. |
| 1607 | BL01170B | 19.54 | 5.821e−37 | 251-288 | BL01170 | Ribosomal protein L6e |
| | | | | | | proteins. |
| 1607 | PD01457A | 16.51 | 9.845e−09 | 67-111 | PD01457 | RIBOSOMAL PROTEIN 40S |
| | | | | | | ZINC-FINGER METAL. |
| 1608 | BL50040A | 12.98 | 1.450e−14 | 4-15 | BL50040 | Elongation factor 1 |
| | | | | | | gamma chain profile. |
| 1608 | BL50040B | 13.65 | 7.000e−30 | 53-78 | BL50040 | Elongation factor 1 |
| | | | | | | gamma chain profile. |
| 1608 | BL50040C | 22.62 | 3.739e−38 | 135-177 | BL50040 | Elongation factor 1 |
| | | | | | | gamma chain profile. |
| 1608 | BL50040D | 17.41 | 1.000e−40 | 273-322 | BL50040 | Elongation factor 1 |
| | | | | | | gamma chain profile. |
| 1608 | BL50040E | 18.79 | 1.000e−40 | 327-381 | BL50040 | Elongation factor 1 |
| | | | | | | gamma chain profile. |
| 1608 | BL50040F | 18.99 | 5.320e−40 | 384-421 | BL50040 | Elongation factor 1 |
| | | | | | | gamma chain profile. |
| 1608 | BL50040G | 8.15 | 1.900e−13 | 427-437 | BL50040 | Elongation factor 1 |
| | | | | | | gamma chain profile. |
| 1608 | DM01181C | 17.27 | 6.055e−12 | 125-162 | DM01181 | 7 kw 103-1A |
| | | | | | | LACTOYLGLUTATHIONE |
| | | | | | | GLUTATHIONE LYASE. |
| 1608 | PF00043 | 21.83 | 9.735e−11 | 57-86 | PF00043 | Glutathione S- |
| | | | | | | transferases. |
| 1609 | BL01052A | 16.12 | 2.875e−35 | 3-34 | BL01052 | Calponin family repeat |
| | | | | | | proteins. |
| 1609 | BL01052B | 15.31 | 5.219e−26 | 52-77 | BL01052 | Calponin family repeat |
| | | | | | | proteins. |
| 1609 | BL01052C | 18.51 | 1.000e−40 | 88-127 | BL01052 | Calponin family repeat |
| | | | | | | proteins. |
| 1609 | BL01052D | 10.26 | 4.462e−24 | 175-194 | BL01052 | Calponin family repeat |
| | | | | | | proteins. |
| 1609 | PR00888A | 11.87 | 7.750e−18 | 3-17 | PR00888 | SMOOTH MUSCLE |
| | | | | | | PROTEIN/CALPONIN FAMILY |
| | | | | | | SIGNATURE |
| 1609 | PR00888B | 13.72 | 1.321e−14 | 22-35 | PR00888 | SMOOTH MUSCLE |
| | | | | | | PROTEIN/CALPONIN FAMILY |
| | | | | | | SIGNATURE |
| 1609 | PR00888C | 12.27 | 2.286e−17 | 52-67 | PR00888 | SMOOTH MUSCLE |
| | | | | | | PROTEIN/CALPONIN FAMILY |
| | | | | | | SIGNATURE |
| 1609 | PR00888D | 16.09 | 9.550e−19 | 89-105 | PR00888 | SMOOTH MUSCLE |
| | | | | | | PROTEIN/CALPONIN FAMILY |
| | | | | | | SIGNATURE |
| 1609 | PR00888E | 11.81 | 2.800e−18 | 105-120 | PR00888 | SMOOTH MUSCLE |
| | | | | | | PROTEIN/CALPONIN FAMILY |
| | | | | | | SIGNATURE |
| 1609 | PR00888F | 7.44 | 4.600e−18 | 126-140 | PR00888 | SMOOTH MUSCLE |
| | | | | | | PROTEIN/CALPONIN FAMILY |
| | | | | | | SIGNATURE |
| 1609 | PR00888G | 12.73 | 9.438e−15 | 163-176 | PR00888 | SMOOTH MUSCLE |
| | | | | | | PROTEIN/CALPONIN FAMILY |
| | | | | | | SIGNATURE |
| 1609 | PR00888H | 9.97 | 8.269e−20 | 176-191 | PR00888 | SMOOTH MUSCLE |
| | | | | | | PROTEIN/CALPONIN FAMILY |
| | | | | | | SIGNATURE |
| 1609 | PR00889E | 12.18 | 7.300e−11 | 172-187 | PR00889 | CALPONIN SIGNATURE |
| 1609 | PR00890A | 8.61 | 1.000e−26 | 34-53 | PR00890 | SMOOTH MUSCLE PROTEIN |
| | | | | | | 22-ALPHA (TRANSGELIN) |
| | | | | | | SIGNATURE |
| 1609 | PR00890B | 8.75 | 6.318e−19 | 62-77 | PR00890 | SMOOTH MUSCLE PROTEIN |
| | | | | | | 22-ALPHA (TRANSGELIN) |
| | | | | | | SIGNATURE |
| 1609 | PR00890C | 8.22 | 1.600e−19 | 85-98 | PR00890 | SMOOTH MUSCLE PROTEIN |
| | | | | | | 22-ALPHA (TRANSGELIN) |
| | | | | | | SIGNATURE |
| 1609 | PR00890D | 16.17 | 1.130e−13 | 119-128 | PR00890 | SMOOTH MUSCLE PROTEIN |
| | | | | | | 22-ALPHA (TRANSGELIN) |
| | | | | | | SIGNATURE |
| 1609 | PR00890E | 14.34 | 1.429e−27 | 136-155 | PR00890 | SMOOTH MUSCLE PROTEIN |
| | | | | | | 22-ALPHA (TRANSGELIN) |
| | | | | | | SIGNATURE |
| 1609 | PR00890F | 12.92 | 1.643e−17 | 162-174 | PR00890 | SMOOTH MUSCLE PROTEIN |
| | | | | | | 22-ALPHA (TRANSGELIN) |
| | | | | | | SIGNATURE |
| 1612 | PR00194A | 7.86 | 7.652e−09 | 180-197 | PR00194 | TROPOMYOSIN SIGNATURE |
| 1614 | DM00934A | 20.07 | 8.663e−11 | 7-54 | DM00934 | kw DIHYDROFLAVONOL |
| | | | | | | YOL151W YDR541C |
| | | | | | | YGL157W. |
| 1616 | BL00605 | 27.67 | 1.000e−40 | 74-127 | BL00605 | ATP synthase c subunit |
| | | | | | | proteins. |
| 1616 | PR00124A | 8.81 | 2.125e−17 | 70-89 | PR00124 | ATP SYNTHASE C SUBUNIT |
| | | | | | | SIGNATURE |
| 1616 | PR00124A | 8.81 | 8.603e−09 | 72-91 | PR00124 | ATP SYNTHASE C SUBUNIT |
| | | | | | | SIGNATURE |
| 1616 | PR00124B | 14.66 | 1.000e−15 | 91-106 | PR00124 | ATP SYNTHASE C SUBUNIT |
| | | | | | | SIGNATURE |
| 1616 | PR00124C | 12.42 | 3.250e−27 | 108-133 | PR00124 | ATP SYNTHASE C SUBUNIT |
| | | | | | | SIGNATURE |
| 1618 | BL00310E | 19.92 | 8.875e−12 | 257-281 | BL00310 | Lysosome-associated |
| | | | | | | membrane glycoproteins |
| | | | | | | duplicated domain |
| | | | | | | proteins. |
| 1618 | BL00310F | 23.26 | 3.377e−10 | 291-345 | BL00310 | Lysosome-associated |
| | | | | | | membrane glycoproteins |
| | | | | | | duplicated domain |
| | | | | | | proteins. |
| 1618 | PR00336A | 12.86 | 6.455e−24 | 158-182 | PR00336 | LYSOSOME-ASSOCIATED |
| | | | | | | MEMBRANE GLYCOPROTEIN |
| | | | | | | SIGNATURE |
| 1618 | PR00336B | 15.42 | 9.027e−13 | 269-283 | PR00336 | LYSOSOME-ASSOCIATED |
| | | | | | | MEMBRANE GLYCOPROTEIN |
| | | | | | | SIGNATURE |
| 1618 | PR00336C | 14.47 | 9.438e−11 | 306-318 | PR00336 | LYSOSOME-ASSOCIATED |
| | | | | | | MEMBRANE GLYCOPROTEIN |
| | | | | | | SIGNATURE |
| 1618 | PR00336D | 9.96 | 5.705e−19 | 319-341 | PR00336 | LYSOSOME-ASSOCIATED |
| | | | | | | MEMBRANE GLYCOPROTEIN |
| | | | | | | SIGNATURE |
| 1618 | PR00336E | 11.59 | 1.711e−12 | 341-353 | PR00336 | LYSOSOME-ASSOCIATED |
| | | | | | | MEMBRANE GLYCOPROTEIN |
| | | | | | | SIGNATURE |
| 1619 | BL00280 | 24.61 | 5.091e−38 | 317-360 | BL00280 | Pancreatic trypsin |
| | | | | | | inhibitor (Kunitz) |
| | | | | | | family proteins. |
| 1619 | BL00319A | 17.62 | 1.000e−40 | 77-125 | BL00319 | Amyloidogenic |
| | | | | | | glycoprotein |
| | | | | | | extracellular domain |
| | | | | | | proteins. |
| 1619 | BL00319B | 17.02 | 2.440e−29 | 173-198 | BL00319 | Amyloidogenic |
| | | | | | | glycoprotein |
| | | | | | | extracellular domain |
| | | | | | | proteins. |
| 1619 | BL00319C | 17.12 | 6.143e−30 | 265-298 | BL00319 | Amyloidogenic |
| | | | | | | glycoprotein |
| | | | | | | extracellular domain |
| | | | | | | proteins. |
| 1619 | BL00319D | 21.29 | 1.000e−40 | 365-414 | BL00319 | Amyloidogenic |
| | | | | | | glycoprotein |
| | | | | | | extracellular domain |
| | | | | | | proteins. |
| 1619 | BL00319E | 12.66 | 1.000e−40 | 438-479 | BL00319 | Amyloidogenic |
| | | | | | | glycoprotein |
| | | | | | | extracellular domain |
| | | | | | | proteins. |
| 1619 | BL00319F | 13.95 | 1.000e−40 | 480-522 | BL00319 | Amyloidogenic |
| | | | | | | glycoprotein |
| | | | | | | extracellular domain |
| | | | | | | proteins. |
| 1619 | BL00319G | 16.93 | 1.000e−40 | 717-762 | BL00319 | Amyloidogenic |
| | | | | | | glycoprotein |
| | | | | | | extracellular domain |
| | | | | | | proteins. |
| 1619 | PR00203A | 13.98 | 3.829e−21 | 193-211 | PR00203 | AMYLOID A4 PROTEIN |
| | | | | | | PRECURSOR SIGNATURE |
| 1619 | PR00203B | 11.38 | 8.500e−28 | 381-404 | PR00203 | AMYLOID A4 PROTEIN |
| | | | | | | PRECURSOR SIGNATURE |
| 1619 | PR00203C | 6.37 | 4.667e−27 | 692-717 | PR00203 | AMYLOID A4 PROTEIN |
| | | | | | | PRECURSOR SIGNATURE |
| 1619 | PR00203D | 13.57 | 2.895e−30 | 738-760 | PR00203 | AMYLOID A4 PROTEIN |
| | | | | | | PRECURSOR SIGNATURE |
| 1619 | PR00759A | 14.51 | 2.440e−10 | 307-321 | PR00759 | BASIC PROTEASE (KUNITZ- |
| | | | | | | TYPE) INHIBITOR FAMILY |
| | | | | | | SIGNATURE |
| 1619 | PR00759B | 11.26 | 7.000e−13 | 335-345 | PR00759 | BASIC PROTEASE (KUNITZ- |
| | | | | | | TYPE) INHIBITOR FAMILY |
| | | | | | | SIGNATURE |
| 1619 | PR00759C | 14.15 | 1.000e−14 | 345-360 | PR00759 | BASIC PROTEASE (KUNITZ- |
| | | | | | | TYPE) INHIBITOR FAMILY |
| | | | | | | SIGNATURE |
| 1620 | BL00634 | 34.38 | 1.333e−37 | 93-143 | BL00634 | Ribosomal protein L30 |
| | | | | | | proteins. |
| 1622 | BL00107A | 18.39 | 9.400e−22 | 1012-1042 | BL00107 | Protein kinases ATP- |
| | | | | | | binding region |
| | | | | | | proteins. |
| 1622 | BL00107B | 13.31 | 7.300e−14 | 1079-1094 | BL00107 | Protein kinases ATP- |
| | | | | | | binding region |
| | | | | | | proteins. |
| 1622 | BL00239C | 18.75 | 1.913e−10 | 999-1021 | BL00239 | Receptor tyrosine |
| | | | | | | kinase class II |
| | | | | | | proteins. |
| 1622 | BL00239D | 16.81 | 9.043e−14 | 1024-1049 | BL00239 | Receptor tyrosine |
| | | | | | | kinase class II |
| | | | | | | proteins. |
| 1622 | BL00239E | 17.14 | 4.436e−32 | 1051-1100 | BL00239 | Receptor tyrosine |
| | | | | | | kinase class II |
| | | | | | | proteins. |
| 1622 | BL00239F | 28.15 | 2.770e−17 | 1106-1150 | BL00239 | Receptor tyrosine |
| | | | | | | kinase class II |
| | | | | | | proteins. |
| 1622 | BL00240A | 22.72 | 7.500e−18 | 32-60 | BL00240 | Receptor tyrosine |
| | | | | | | kinase class III |
| | | | | | | proteins. |
| 1622 | BL00240B | 24.70 | 5.333e−15 | 575-598 | BL00240 | Receptor tyrosine |
| | | | | | | kinase class III |
| | | | | | | proteins. |
| 1622 | BL00240B | 24.70 | 5.404e−09 | 452-475 | BL00240 | Receptor tyrosine |
| | | | | | | kinase class III |
| | | | | | | proteins. |
| 1622 | BL00240B | 24.70 | 6.651e−10 | 680-703 | BL00240 | Receptor tyrosine |
| | | | | | | kinase class III |
| | | | | | | proteins. |
| 1622 | BL00240C | 22.58 | 1.000e−40 | 819-867 | BL00240 | Receptor tyrosine |
| | | | | | | kinase class III |
| | | | | | | proteins. |
| 1622 | BL00240D | 23.07 | 1.000e−40 | 868-922 | BL00240 | Receptor tyrosine |
| | | | | | | kinase class III |
| | | | | | | proteins. |
| 1622 | BL00240E | 11.56 | 3.182e−40 | 998-1035 | BL00240 | Receptor tyrosine |
| | | | | | | kinase class III |
| | | | | | | proteins. |
| 1622 | BL00240F | 17.74 | 1.000e−40 | 1050-1097 | BL00240 | Receptor tyrosine |
| | | | | | | kinase class III |
| | | | | | | proteins. |
| 1622 | BL00240G | 28.45 | 1.000e−40 | 1098-1150 | BL00240 | Receptor tyrosine |
| | | | | | | kinase class III |
| | | | | | | proteins. |
| 1622 | BL00790M | 8.74 | 1.692e−09 | 1003-1024 | BL00790 | Receptor tyrosine |
| | | | | | | kinase class V |
| | | | | | | proteins. |
| 1622 | BL00790N | 13.25 | 6.929e−13 | 1025-1051 | BL00790 | Receptor tyrosine |
| | | | | | | kinase class V |
| | | | | | | proteins. |
| 1622 | BL00790O | 7.68 | 4.539e−14 | 1058-1090 | BL00790 | Receptor tyrosine |
| | | | | | | kinase class V |
| | | | | | | proteins. |
| 1622 | BL00790Q | 15.61 | 8.533e−15 | 1117-1165 | BL00790 | Receptor tyrosine |
| | | | | | | kinase class V |
| | | | | | | proteins. |
| 1622 | BL50001B | 17.40 | 1.000e−12 | 1009-1029 | BL50001 | Src homology 2 (SH2) |
| | | | | | | domain proteins |
| | | | | | | profile. |
| 1622 | BL50001D | 11.00 | 9.100e−10 | 1079-1089 | BL50001 | Src homology 2 (SH2) |
| | | | | | | domain proteins |
| | | | | | | profile. |
| 1622 | DM00179 | 13.97 | 8.826e−09 | 304-313 | DM00179 | w KINASE ALPHA ADHESION |
| | | | | | | T-CELL. |
| 1622 | PD02870B | 18.83 | 9.521e−09 | 621-653 | PD02870 | RECEPTOR INTERLEUKIN-1 |
| | | | | | | PRECURSOR. |
| 1622 | PD02870B | 18.83 | 9.617e−09 | 716-748 | PD02870 | RECEPTOR INTERLEUKIN-1 |
| | | | | | | PRECURSOR. |
| 1622 | PD02870D | 15.74 | 1.200e−10 | 621-655 | PD02870 | RECEPTOR INTERLEUKIN-1 |
| | | | | | | PRECURSOR. |
| 1622 | PD02870D | 15.74 | 5.117e−09 | 390-424 | PD02870 | RECEPTOR INTERLEUKIN-1 |
| | | | | | | PRECURSOR. |
| 1622 | PR00109A | 15.00 | 4.808e−11 | 909-922 | PR00109 | TYROSINE KINASE |
| | | | | | | CATALYTIC DOMAIN |
| | | | | | | SIGNATURE |
| 1622 | PR00109B | 12.27 | 5.714e−19 | 1012-1030 | PR00109 | TYROSINE KINASE |
| | | | | | | CATALYTIC DOMAIN |
| | | | | | | SIGNATURE |
| 1622 | PR00109C | 12.85 | 3.118e−12 | 1061-1071 | PR00109 | TYROSINE KINASE |
| | | | | | | CATALYTIC DOMAIN |
| | | | | | | SIGNATURE |
| 1622 | PR00109D | 17.04 | 2.286e−25 | 1080-1102 | PR00109 | TYROSINE KINASE |
| | | | | | | CATALYTIC DOMAIN |
| | | | | | | SIGNATURE |
| 1622 | PR00109E | 14.41 | 7.857e−19 | 1125-1147 | PR00109 | TYROSINE KINASE |
| | | | | | | CATALYTIC DOMAIN |
| | | | | | | SIGNATURE |
| 1622 | PR00573D | 15.57 | 1.000e−09 | 1284-1299 | PR00573 | INTERLEUKIN 8B RECEPTOR |
| | | | | | | SIGNATURE |
| 1623 | DM01354Z | 9.06 | 5.747e−14 | 1-49 | DM01354 | kw TRANSCRIPTASE |
| | | | | | | REVERSE II ORF2. |
| 1624 | BL00262A | 12.48 | 8.200e−09 | 523-540 | BL00262 | Insulin family |
| | | | | | | proteins. |
| 1624 | BL01282B | 30.49 | 5.243e−09 | 517-555 | BL01282 | BIR repeat proteins. |
| 1625 | BL00779A | 14.01 | 4.667e−27 | 31-57 | BL00779 | Glycoprotein hormones |
| | | | | | | alpha chain proteins. |
| 1625 | BL00779B | 6.45 | 1.429e−20 | 58-73 | BL00779 | Glycoprotein hormones |
| | | | | | | alpha chain proteins. |
| 1625 | BL00779C | 14.39 | 1.333e−37 | 82-115 | BL00779 | Glycoprotein hormones |
| | | | | | | alpha chain proteins. |
| 1625 | PR00274A | 9.96 | 2.500e−15 | 30-44 | PR00274 | GLYCOPROTEIN HORMONE |
| | | | | | | ALPHA CHAIN SIGNATURE |
| 1625 | PR00274B | 9.15 | 8.091e−26 | 50-69 | PR00274 | GLYCOPROTEIN HORMONE |
| | | | | | | ALPHA CHAIN SIGNATURE |
| 1625 | PR00274C | 13.49 | 5.959e−22 | 70-87 | PR00274 | GLYCOPROTEIN HORMONE |
| | | | | | | ALPHA CHAIN SIGNATURE |
| 1625 | PR00274D | 9.71 | 1.900e−23 | 99-115 | PR00274 | GLYCOPROTEIN HORMONE |
| | | | | | | ALPHA CHAIN SIGNATURE |
| 1626 | BL01019A | 13.20 | 7.353e−10 | 203-242 | BL01019 | ADP-ribosylation |
| | | | | | | factors family |
| | | | | | | proteins. |
| 1626 | PR00318A | 7.84 | 4.000e−18 | 42-57 | PR00318 | ALPHA G-PROTEIN |
| | | | | | | (TRANSDUCIN) SIGNATURE |
| 1626 | PR00318B | 14.79 | 4.429e−22 | 190-212 | PR00318 | ALPHA G-PROTEIN |
| | | | | | | (TRANSDUCIN) SIGNATURE |
| 1626 | PR00318C | 12.09 | 9.526e−22 | 219-236 | PR00318 | ALPHA G-PROTEIN |
| | | | | | | (TRANSDUCIN) SIGNATURE |
| 1626 | PR00318D | 16.28 | 3.625e−26 | 241-269 | PR00318 | ALPHA G-PROTEIN |
| | | | | | | (TRANSDUCIN) SIGNATURE |
| 1626 | PR00318E | 7.23 | 2.875e−11 | 287-296 | PR00318 | ALPHA G-PROTEIN |
| | | | | | | (TRANSDUCIN) SIGNATURE |
| 1626 | PR00440A | 9.18 | 9.416e−14 | 42-57 | PR00440 | G-PROTEIN ALPHA SUBUNIT |
| | | | | | | GROUP 12 SIGNATURE |
| 1626 | PR00440B | 13.27 | 7.078e−13 | 190-212 | PR00440 | G-PROTEIN ALPHA SUBUNIT |
| | | | | | | GROUP 12 SIGNATURE |
| 1626 | PR00440C | 9.54 | 7.197e−15 | 219-236 | PR00440 | G-PROTEIN ALPHA SUBUNIT |
| | | | | | | GROUP 12 SIGNATURE |
| 1626 | PR00440D | 8.15 | 5.081e−16 | 241-269 | PR00440 | G-PROTEIN ALPHA SUBUNIT |
| | | | | | | GROUP 12 SIGNATURE |
| 1626 | PR00440E | 11.16 | 3.160e−10 | 288-296 | PR00440 | G-PROTEIN ALPHA SUBUNIT |
| | | | | | | GROUP 12 SIGNATURE |
| 1626 | PR00441A | 10.69 | 5.725e−17 | 42-57 | PR00441 | G-PROTEIN ALPHA SUBUNIT |
| | | | | | | GROUP I SIGNATURE |
| 1626 | PR00441B | 16.16 | 7.709e−18 | 190-212 | PR00441 | G-PROTEIN ALPHA SUBUNIT |
| | | | | | | GROUP I SIGNATURE |
| 1626 | PR00441C | 14.17 | 5.245e−16 | 219-236 | PR00441 | G-PROTEIN ALPHA SUBUNIT |
| | | | | | | GROUP I SIGNATURE |
| 1626 | PR00441D | 14.44 | 9.847e−20 | 241-269 | PR00441 | G-PROTEIN ALPHA SUBUNIT |
| | | | | | | GROUP I SIGNATURE |
| 1626 | PR00441E | 8.35 | 8.377e−10 | 288-296 | PR00441 | G-PROTEIN ALPHA SUBUNIT |
| | | | | | | GROUP I SIGNATURE |
| 1626 | PR00442A | 6.46 | 6.865e−13 | 42-57 | PR00442 | G-PROTEIN ALPHA SUBUNIT |
| | | | | | | GROUP Q SIGNATURE |
| 1626 | PR00442B | 7.17 | 5.390e−13 | 190-213 | PR00442 | G-PROTEIN ALPHA SUBUNIT |
| | | | | | | GROUP Q SIGNATURE |
| 1626 | PR00442C | 17.61 | 5.985e−16 | 219-236 | PR00442 | G-PROTEIN ALPHA SUBUNIT |
| | | | | | | GROUP Q SIGNATURE |
| 1626 | PR00442D | 9.29 | 9.679e−13 | 241-269 | PR00442 | G-PROTEIN ALPHA SUBUNIT |
| | | | | | | GROUP Q SIGNATURE |
| 1626 | PR00442E | 7.23 | 1.273e−10 | 288-296 | PR00442 | G-PROTEIN ALPHA SUBUNIT |
| | | | | | | GROUP Q SIGNATURE |
| 1626 | PR00443A | 15.16 | 1.097e−19 | 42-57 | PR00443 | G-PROTEIN ALPHA SUBUNIT |
| | | | | | | GROUP S SIGNATURE |
| 1626 | PR00443B | 9.25 | 3.000e−29 | 94-117 | PR00443 | G-PROTEIN ALPHA SUBUNIT |
| | | | | | | GROUP S SIGNATURE |
| 1626 | PR00443C | 14.35 | 1.346e−29 | 190-212 | PR00443 | G-PROTEIN ALPHA SUBUNIT |
| | | | | | | GROUP S SIGNATURE |
| 1626 | PR00443D | 13.15 | 1.205e−25 | 219-236 | PR00443 | G-PROTEIN ALPHA SUBUNIT |
| | | | | | | GROUP S SIGNATURE |
| 1626 | PR00443E | 11.98 | 2.421e−36 | 241-269 | PR00443 | G-PROTEIN ALPHA SUBUNIT |
| | | | | | | GROUP S SIGNATURE |
| 1626 | PR00443F | 12.68 | 1.092e−11 | 288-296 | PR00443 | G-PROTEIN ALPHA SUBUNIT |
| | | | | | | GROUP S SIGNATURE |
| 1626 | PR00443G | 7.19 | 1.750e−29 | 334-356 | PR00443 | G-PROTEIN ALPHA SUBUNIT |
| | | | | | | GROUP S SIGNATURE |
| 1627 | BL00030A | 14.39 | 1.000e−12 | 16-34 | BL00030 | Eukaryotic RNA-binding |
| | | | | | | region RNP-1 proteins. |
| 1627 | BL00030A | 14.39 | 1.500e−10 | 107-125 | BL00030 | Eukaryotic RNA-binding |
| | | | | | | region RNP-1 proteins. |
| 1627 | BL00030B | 7.03 | 1.900e−11 | 146-155 | BL00030 | Eukaryotic RNA-binding |
| | | | | | | region RNP-1 proteins. |
| 1628 | BL00353C | 14.83 | 4.814e−09 | 1-47 | BL00353 | HMG1/2 proteins. |
| 1628 | PR00901L | 11.74 | 5.348e−09 | 77-90 | PR00901 | PHEROMONE B ALPHA-1 |
| | | | | | | RECEPTOR SIGNATURE |
| 1629 | BL01082 | 20.37 | 7.750e−40 | 152-191 | BL01082 | Ribosomal protein L7Ae |
| | | | | | | proteins. |
| 1629 | PR00881A | 9.94 | 7.517e−13 | 136-150 | PR00881 | RIBOSOMAL PROTEIN |
| | | | | | | L7A/RS6 FAMILY |
| | | | | | | SIGNATURE |
| 1629 | PR00881B | 9.01 | 4.000e−17 | 155-168 | PR00881 | RIBOSOMAL PROTEIN |
| | | | | | | L7A/RS6 FAMILY |
| | | | | | | SIGNATURE |
| 1629 | PR00881C | 12.76 | 1.000e−13 | 171-181 | PR00881 | RIBOSOMAL PROTEIN |
| | | | | | | L7A/RS6 FAMILY |
| | | | | | | SIGNATURE |
| 1629 | PR00881D | 11.60 | 8.500e−16 | 181-195 | PR00881 | RIBOSOMAL PROTEIN |
| | | | | | | L7A/RS6 FAMILY |
| | | | | | | SIGNATURE |
| 1629 | PR00882A | 8.80 | 3.160e−22 | 36-53 | PR00882 | RIBOSOMAL PROTEIN L7A |
| | | | | | | FAMILY SIGNATURE |
| 1629 | PR00882B | 9.47 | 1.000e−22 | 53-70 | PR00882 | RIBOSOMAL PROTEIN L7A |
| | | | | | | FAMILY SIGNATURE |
| 1629 | PR00882C | 15.18 | 2.969e−23 | 73-92 | PR00882 | RIBOSOMAL PROTEIN L7A |
| | | | | | | FAMILY SIGNATURE |
| 1629 | PR00882D | 14.85 | 6.500e−17 | 97-110 | PR00882 | RIBOSOMAL PROTEIN L7A |
| | | | | | | FAMILY SIGNATURE |
| 1629 | PR00882E | 14.99 | 2.216e−21 | 132-152 | PR00882 | RIBOSOMAL PROTEIN L7A |
| | | | | | | FAMILY SIGNATURE |
| 1629 | PR00882F | 13.81 | 4.273e−26 | 193-217 | PR00882 | RIBOSOMAL PROTEIN L7A |
| | | | | | | FAMILY SIGNATURE |
| 1629 | PR00882G | 10.27 | 8.500e−25 | 225-245 | PR00882 | RIBOSOMAL PROTEIN L7A |
| | | | | | | FAMILY SIGNATURE |
| 1629 | PR00884C | 7.05 | 7.409e−10 | 154-165 | PR00884 | RIBOSOMAL PROTEIN HS6 |
| | | | | | | SIGNATURE |
| 1630 | BL00529A | 22.06 | 1.000e−40 | 5-49 | BL00529 | Ribosomal protein S24e |
| | | | | | | proteins. |
| 1630 | BL00529B | 20.08 | 1.000e−40 | 55-99 | BL00529 | Ribosomal protein S24e |
| | | | | | | proteins. |
| 1631 | BL00612A | 12.09 | 7.171e−09 | 3-18 | BL00612 | Osteonectin domain |
| | | | | | | proteins. |
| 1631 | PD01469 | 20.59 | 1.346e−29 | 357-388 | PD01469 | GLYCOPROTEIN PROTEIN |
| | | | | | | PRECURSOR SA. |
| 1631 | PD01469 | 20.59 | 7.176e−19 | 451-482 | PD01469 | GLYCOPROTEIN PROTEIN |
| | | | | | | PRECURSOR SA. |
| 1632 | BL00326A | 14.01 | 6.651e−09 | 162-195 | BL00326 | Tropomyosins proteins. |
| 1632 | BL00326A | 14.01 | 9.308e−37 | 1-34 | BL00326 | Tropomyosins proteins. |
| 1632 | BL00326B | 7.68 | 1.000e−40 | 81-129 | BL00326 | Tropomyosins proteins. |
| 1632 | BL00326B | 7.68 | 3.838e−10 | 39-87 | BL00326 | Tropomyosins proteins. |
| 1632 | BL00326C | 9.99 | 1.000e−40 | 137-190 | BL00326 | Tropomyosins proteins. |
| 1632 | BL00326C | 9.99 | 2.116e−09 | 95-148 | BL00326 | Tropomyosins proteins. |
| 1632 | BL00326C | 9.99 | 3.306e−09 | 193-246 | BL00326 | Tropomyosins proteins. |
| 1632 | BL00326D | 8.76 | 1.000e−40 | 220-260 | BL00326 | Tropomyosins proteins. |
| 1632 | BL00326D | 8.76 | 7.511e−12 | 59-99 | BL00326 | Tropomyosins proteins. |
| 1632 | PR00194A | 7.86 | 4.857e−21 | 84-101 | PR00194 | TROPOMYOSIN SIGNATURE |
| 1632 | PR00194B | 10.24 | 6.143e−26 | 120-140 | PR00194 | TROPOMYOSIN SIGNATURE |
| 1632 | PR00194C | 6.38 | 1.450e−32 | 145-173 | PR00194 | TROPOMYOSIN SIGNATURE |
| 1632 | PR00194D | 9.57 | 1.788e−09 | 21-44 | PR00194 | TROPOMYOSIN SIGNATURE |
| 1632 | PR00194D | 9.57 | 6.400e−23 | 175-198 | PR00194 | TROPOMYOSIN SIGNATURE |
| 1632 | PR00194E | 8.74 | 1.000e−30 | 231-256 | PR00194 | TROPOMYOSIN SIGNATURE |
| 1633 | BL00266A | 15.69 | 5.200e−26 | 46-72 | BL00266 | Somatotropin, prolactin |
| | | | | | | and related hormones |
| | | | | | | proteins. |
| 1633 | BL00266B | 24.48 | 1.857e−29 | 86-123 | BL00266 | Somatotropin, prolactin |
| | | | | | | and related hormones |
| | | | | | | proteins. |
| 1633 | BL00266C | 13.66 | 5.304e−12 | 146-162 | BL00266 | Somatotropin, prolactin |
| | | | | | | and related hormones |
| | | | | | | proteins |
| 1633 | BL00266D | 12.72 | 1.818e−27 | 197-220 | BL00266 | Somatotropin, prolactin |
| | | | | | | and related hormones |
| | | | | | | proteins. |
| 1633 | PR00836A | 14.40 | 9.471e−14 | 86-99 | PR00836 | SOMATOTROPIN HORMONE |
| | | | | | | FAMILY SIGNATURE |
| 1633 | PR00836B | 16.59 | 3.571e−15 | 108-126 | PR00836 | SOMATOTROPIN HORMONE |
| | | | | | | FAMILY SIGNATURE |
| 1633 | PR00836C | 11.95 | 2.500e−17 | 190-206 | PR00836 | SOMATOTROPIN HORMONE |
| | | | | | | FAMILY SIGNATURE |
| 1633 | PR00836D | 13.05 | 2.688e−17 | 206-220 | PR00836 | SOMATOTROPIN HORMONE |
| | | | | | | FAMILY SIGNATURE |
| 1635 | BL00383A | 13.34 | 6.000e−09 | 1657-1671 | BL00383 | Tyrosine specific |
| | | | | | | protein phosphatases |
| | | | | | | proteins. |
| 1635 | BL00383A | 13.34 | 9.550e−14 | 1368-1382 | BL00383 | Tyrosine specific |
| | | | | | | protein phosphatases |
| | | | | | | proteins. |
| 1635 | BL00383B | 7.61 | 6.143e−12 | 1393-1401 | BL00383 | Tyrosine specific |
| | | | | | | protein phosphatases |
| | | | | | | proteins. |
| 1635 | BL00383B | 7.61 | 8.500e−09 | 1682-1690 | BL00383 | Tyrosine specific |
| | | | | | | protein phosphatases |
| | | | | | | proteins. |
| 1635 | BL00383C | 10.10 | 1.000e−10 | 1712-1722 | BL00383 | Tyrosine specific |
| | | | | | | protein phosphatases |
| | | | | | | proteins. |
| 1635 | BL00383C | 10.10 | 4.500e−13 | 1423-1433 | BL00383 | Tyrosine specific |
| | | | | | | protein phosphatases |
| | | | | | | proteins. |
| 1635 | BL00383D | 11.92 | 7.500e−12 | 1787-1799 | BL00383 | Tyrosine specific |
| | | | | | | protein phosphatases |
| | | | | | | proteins. |
| 1635 | BL00383D | 11.92 | 9.400e−13 | 1498-1510 | BL00383 | Tyrosine specific |
| | | | | | | protein phosphatases |
| | | | | | | proteins. |
| 1635 | BL00383E | 10.35 | 1.000e−14 | 1536-1546 | BL00383 | Tyrosine specific |
| | | | | | | protein phosphatases |
| | | | | | | proteins. |
| 1635 | BL00383E | 10.35 | 1.000e−14 | 1827-1837 | BL00383 | Tyrosine specific |
| | | | | | | protein phosphatases |
| | | | | | | proteins. |
| 1635 | BL00383F | 15.51 | 7.120e−13 | 1865-1880 | BL00383 | Tyrosine specific |
| | | | | | | protein phosphatases |
| | | | | | | proteins. |
| 1635 | BL00383F | 15.51 | 8.941e−16 | 1574-1589 | BL00383 | Tyrosine specific |
| | | | | | | protein phosphatases |
| | | | | | | proteins. |
| 1635 | BL00621G | 22.33 | 1.250e−10 | 1225-1278 | BL00621 | Tissue factor proteins. |
| 1635 | BL00790I | 20.01 | 1.536e−09 | 663-693 | BL00790 | Receptor tyrosine |
| | | | | | | kinase class V |
| | | | | | | proteins. |
| 1635 | BL00790I | 20.01 | 2.688e−10 | 776-806 | BL00790 | Receptor tyrosine |
| | | | | | | kinase class V |
| | | | | | | proteins. |
| 1635 | BL00790I | 20.01 | 2.800e−10 | 467-497 | BL00790 | Receptor tyrosine |
| | | | | | | kinase class V |
| | | | | | | proteins. |
| 1635 | BL00790I | 20.01 | 8.500e−12 | 561-591 | BL00790 | Receptor tyrosine |
| | | | | | | kinase class V |
| | | | | | | proteins. |
| 1635 | BL00790I | 20.01 | 9.735e−13 | 967-997 | BL00790 | Receptor tyrosine |
| | | | | | | kinase class V |
| | | | | | | proteins. |
| 1635 | PR00014B | 14.77 | 5.500e−09 | 431-441 | PR00014 | FIBRONECTIN TYPE III |
| | | | | | | REPEAT SIGNATURE |
| 1635 | PR00014C | 15.44 | 5.500e−10 | 550-568 | PR00014 | FIBRONECTIN TYPE III |
| | | | | | | REPEAT SIGNATURE |
| 1635 | PR00014C | 15.44 | 8.500e−14 | 956-974 | PR00014 | FIBRONECTIN TYPE III |
| | | | | | | REPEAT SIGNATURE |
| 1635 | PR00014D | 12.04 | 5.235e−10 | 878-892 | PR00014 | FIBRONECTIN TYPE III |
| | | | | | | REPEAT SIGNATURE |
| 1635 | PR00014D | 12.04 | 7.000e−11 | 670-684 | PR00014 | FIBRONECTIN TYPE III |
| | | | | | | REPEAT SIGNATURE |
| 1635 | PR00014D | 12.04 | 7.750e−09 | 974-988 | PR00014 | FIBRONECTIN TYPE III |
| | | | | | | REPEAT SIGNATURE |
| 1635 | PR00219C | 9.04 | 4.450e−09 | 1251-1270 | PR00219 | SYNAPTOBREVIN SIGNATURE |
| 1635 | PR00346H | 10.74 | 1.214e−09 | 1250-1273 | PR00346 | TISSUE FACTOR SIGNATURE |
| 1635 | PR00700A | 6.96 | 8.714e−11 | 1394-1401 | PR00700 | PROTEIN TYROSINE |
| | | | | | | PHOSPHATASE SIGNATURE |
| 1635 | PR00700B | 16.80 | 1.500e−24 | 1410-1430 | PR00700 | PROTEIN TYROSINE |
| | | | | | | PHOSPHATASE SIGNATURE |
| 1635 | PR00700B | 16.80 | 4.240e−21 | 1699-1719 | PR00700 | PROTEIN TYROSINE |
| | | | | | | PHOSPHATASE SIGNATURE |
| 1635 | PR00700C | 13.17 | 5.800e−18 | 1494-1511 | PR00700 | PROTEIN TYROSINE |
| | | | | | | PHOSPHATASE SIGNATURE |
| 1635 | PR00700C | 13.17 | 7.353e−17 | 1783-1800 | PR00700 | PROTEIN TYROSINE |
| | | | | | | PHOSPHATASE SIGNATURE |
| 1635 | PR00700D | 12.47 | 4.214e−22 | 1533-1551 | PR00700 | PROTEIN TYROSINE |
| | | | | | | PHOSPHATASE SIGNATURE |
| 1635 | PR00700D | 12.47 | 7.158e−20 | 1824-1842 | PR00700 | PROTEIN TYROSINE |
| | | | | | | PHOSPHATASE SIGNATURE |
| 1635 | PR00700E | 17.57 | 4.000e−14 | 1855-1870 | PR00700 | PROTEIN TYROSINE |
| | | | | | | PHOSPHATASE SIGNATURE |
| 1635 | PR00700E | 17.57 | 5.304e−12 | 1564-1579 | PR00700 | PROTEIN TYROSINE |
| | | | | | | PHOSPHATASE SIGNATURE |
| 1635 | PR00700F | 11.18 | 1.429e−12 | 1871-1881 | PR00700 | PROTEIN TYROSINE |
| | | | | | | PHOSPHATASE SIGNATURE |
| 1635 | PR00700F | 11.18 | 7.353e−13 | 1580-1590 | PR00700 | PROTEIN TYROSINE |
| | | | | | | PHOSPHATASE SIGNATURE |
| 1636 | BL00152A | 15.38 | 5.154e−21 | 134-159 | BL00152 | ATP synthase alpha and |
| | | | | | | beta subunits proteins. |
| 1636 | BL00152B | 21.40 | 4.000e−32 | 191-228 | BL00152 | ATP synthase alpha and |
| | | | | | | beta subunits proteins. |
| 1636 | BL00152C | 11.41 | 6.250e−12 | 291-302 | BL00152 | ATP synthase alpha and |
| | | | | | | beta subunits proteins. |
| 1636 | BL00152D | 29.00 | 1.000e−40 | 310-354 | BL00152 | ATP synthase alpha and |
| | | | | | | beta subunits proteins. |
| 1636 | BL00152E | 22.68 | 1.000e−32 | 362-399 | BL00152 | ATP synthase alpha and |
| | | | | | | beta subunits proteins. |
| 1637 | BL00018 | 7.41 | 4.130e−09 | 67-79 | BL00018 | EF-hand calcium-binding |
| | | | | | | domain proteins. |
| 1637 | BL00303A | 21.77 | 6.667e−26 | 7-43 | BL00303 | S-100/ICaBP type |
| | | | | | | calcium binding |
| | | | | | | protein. |
| 1637 | BL00303B | 26.15 | 1.000e−24 | 55-91 | BL00303 | S-100/ICaBP type |
| | | | | | | calcium binding |
| | | | | | | protein. |
| 1637 | PR00334B | 8.69 | 8.286e−09 | 90-113 | PR00334 | HMW KININOGEN SIGNATURE |
| 1639 | BL00111A | 9.65 | 3.000e−17 | 16-29 | BL00111 | Phosphoglycerate kinase |
| | | | | | | proteins. |
| 1639 | BL00111B | 12.16 | 7.923e−15 | 58-69 | BL00111 | Phosphoglycerate kinase |
| | | | | | | proteins. |
| 1639 | BL00111C | 15.75 | 6.850e−20 | 74-98 | BL00111 | Phosphoglycerate kinase |
| | | | | | | proteins. |
| 1639 | BL00111D | 16.01 | 9.400e−16 | 114-128 | BL00111 | Phosphoglycerate kinase |
| | | | | | | proteins. |
| 1639 | BL00111E | 11.18 | 1.750e−26 | 159-179 | BL00111 | Phosphoglycerate kinase |
| | | | | | | proteins. |
| 1639 | BL00111F | 14.32 | 1.000e−40 | 186-232 | BL00111 | Phosphoglycerate kinase |
| | | | | | | proteins. |
| 1639 | BL00111G | 26.36 | 3.769e−34 | 253-292 | BL00111 | Phosphoglycerate kinase |
| | | | | | | proteins. |
| 1639 | BL00111H | 20.83 | 3.455e−31 | 317-352 | BL00111 | Phosphoglycerate kinase |
| | | | | | | proteins. |
| 1639 | BL00111I | 9.95 | 1.643e−14 | 390-411 | BL00111 | Phosphoglycerate kinase |
| | | | | | | proteins. |
| 1639 | BL00461B | 14.59 | 1.437e−09 | 201-240 | BL00461 | 6-phosphogluconate |
| | | | | | | dehydrogenase proteins. |
| 1639 | PR00477A | 7.93 | 3.769e−20 | 13-29 | PR00477 | PHOSPHOGLYCERATE KINASE |
| | | | | | | FAMILY SIGNATURE |
| 1639 | PR00477B | 14.50 | 4.857e−22 | 34-56 | PR00477 | PHOSPHOGLYCERATE KINASE |
| | | | | | | FAMILY SIGNATURE |
| 1639 | PR00477C | 14.67 | 6.400e−17 | 114-129 | PR00477 | PHOSPHOGLYCERATE KINASE |
| | | | | | | FAMILY SIGNATURE |
| 1639 | PR00477D | 12.94 | 1.900e−28 | 157-179 | PR00477 | PHOSPHOGLYCERATE KINASE |
| | | | | | | FAMILY SIGNATURE |
| 1639 | PR00477E | 11.95 | 3.500e−22 | 186-208 | PR00477 | PHOSPHOGLYCERATE KINASE |
| | | | | | | FAMILY SIGNATURE |
| 1639 | PR00477F | 7.63 | 8.000e−20 | 209-228 | PR00477 | PHOSPHOGLYCERATE KINASE |
| | | | | | | FAMILY SIGNATURE |
| 1639 | PR00477G | 15.40 | 1.000e−24 | 333-358 | PR00477 | PHOSPHOGLYCERATE KINASE |
| | | | | | | FAMILY SIGNATURE |
| 1639 | PR00477H | 5.93 | 6.000e−15 | 369-380 | PR00477 | PHOSPHOGLYCERATE KINASE |
| | | | | | | FAMILY SIGNATURE |
| 1640 | PR00477I | 8.53 | 4.000e−20 | 392-409 | PR00477 | PHOSPHOGLYCERATE KINASE |
| | | | | | | FAMILY SIGNATURE |
| 1640 | PR00360B | 13.61 | 1.818e−09 | 40-53 | PR00360 | C2 DOMAIN SIGNATURE |
| 1640 | PR00360B | 13.61 | 6.455e−11 | 1193-1206 | PR00360 | C2 DOMAIN SIGNATURE |
| 1641 | BL00115A | 15.44 | 1.000e−40 | 50-88 | BL00115 | Eukaryotic RNA |
| | | | | | | polymerase II |
| | | | | | | heptapeptide repeat |
| | | | | | | proteins. |
| 1641 | BL00115B | 15.42 | 2.957e−36 | 89-120 | BL00115 | Eukaryotic RNA |
| | | | | | | polymerase II |
| | | | | | | heptapeptide repeat |
| | | | | | | proteins. |
| 1641 | BL00115C | 14.01 | 4.750e−24 | 182-203 | BL00115 | Eukaryotic RNA |
| | | | | | | polymerase II |
| | | | | | | heptapeptide repeat |
| | | | | | | proteins. |
| 1641 | BL00115D | 19.94 | 1.000e−40 | 224-264 | BL00115 | Eukaryotic RNA |
| | | | | | | polymerase II |
| | | | | | | heptapeptide repeat |
| | | | | | | proteins. |
| 1641 | BL00115E | 14.13 | 8.560e−31 | 297-325 | BL00115 | Eukaryotic RNA |
| | | | | | | polymerase II |
| | | | | | | heptapeptide repeat |
| | | | | | | proteins. |
| 1641 | BL00115F | 10.77 | 7.107e−36 | 326-356 | BL00115 | Eukaryotic RNA |
| | | | | | | polymerase II |
| | | | | | | heptapeptide repeat |
| | | | | | | proteins. |
| 1641 | BL00115G | 11.65 | 2.000e−34 | 357-384 | BL00115 | Eukaryotic RNA |
| | | | | | | polymerase II |
| | | | | | | heptapeptide repeat |
| | | | | | | proteins. |
| 1641 | BL00115H | 14.34 | 2.286e−35 | 385-417 | BL00115 | Eukaryotic RNA |
| | | | | | | polymerase II |
| | | | | | | heptapeptide repeat |
| | | | | | | proteins. |
| 1641 | BL00115I | 8.33 | 1.000e−40 | 443-497 | BL00115 | Eukaryotic RNA |
| | | | | | | polymerase II |
| | | | | | | heptapeptide repeat |
| | | | | | | proteins. |
| 1641 | BL00115J | 16.71 | 9.143e−31 | 498-523 | BL00115 | Eukaryotic RNA |
| | | | | | | polymerase II |
| | | | | | | heptapeptide repeat |
| | | | | | | proteins. |
| 1641 | BL00115K | 15.03 | 1.000e−40 | 524-565 | BL00115 | Eukaryotic RNA |
| | | | | | | polymerase II |
| | | | | | | heptapeptide repeat |
| | | | | | | proteins. |
| 1641 | BL00115L | 12.25 | 6.464e−37 | 566-597 | BL00115 | Eukaryotic RNA |
| | | | | | | polymerase II |
| | | | | | | heptapeptide repeat |
| | | | | | | proteins. |
| 1641 | BL00115M | 19.19 | 1.000e−40 | 622-664 | BL00115 | Eukaryotic RNA |
| | | | | | | polymerase II |
| | | | | | | heptapeptide repeat |
| | | | | | | proteins. |
| 1641 | BL00115N | 20.57 | 1.000e−40 | 665-713 | BL00115 | Eukaryotic RNA |
| | | | | | | polymerase II |
| | | | | | | heptapeptide repeat |
| | | | | | | proteins. |
| 1641 | BL00115O | 16.76 | 1.000e−40 | 735-784 | BL00115 | Eukaryotic RNA |
| | | | | | | polymerase II |
| | | | | | | heptapeptide repeat |
| | | | | | | proteins. |
| 1641 | BL00115P | 11.54 | 1.000e−40 | 785-824 | BL00115 | Eukaryotic RNA |
| | | | | | | polymerase II |
| | | | | | | heptapeptide repeat |
| | | | | | | proteins. |
| 1641 | BL00115Q | 18.08 | 2.575e−37 | 825-854 | BL00115 | Eukaryotic RNA |
| | | | | | | polymerase II |
| | | | | | | heptapeptide repeat |
| | | | | | | proteins. |
| 1641 | BL00115R | 6.50 | 2.385e−32 | 855-881 | BL00115 | Eukaryotic RNA |
| | | | | | | polymerase II |
| | | | | | | heptapeptide repeat |
| | | | | | | proteins. |
| 1641 | BL00115S | 18.24 | 6.333e−40 | 882-923 | BL00115 | Eukaryotic RNA |
| | | | | | | polymerase II |
| | | | | | | heptapeptide repeat |
| | | | | | | proteins. |
| 1641 | BL00115T | 8.45 | 1.000e−40 | 1083-1116 | BL00115 | Eukaryotic RNA |
| | | | | | | polymerase II |
| | | | | | | heptapeptide repeat |
| | | | | | | proteins. |
| 1641 | BL00115U | 10.34 | 2.688e−26 | 1117-1139 | BL00115 | Eukaryotic RNA |
| | | | | | | polymerase II |
| | | | | | | heptapeptide repeat |
| | | | | | | proteins. |
| 1641 | BL00115V | 21.32 | 1.000e−40 | 1140-1189 | BL00115 | Eukaryotic RNA |
| | | | | | | polymerase II |
| | | | | | | heptapeptide repeat |
| | | | | | | proteins. |
| 1641 | BL00115W | 21.01 | 1.000e−40 | 1208-1253 | BL00115 | Eukaryotic RNA |
| | | | | | | polymerase II |
| | | | | | | heptapeptide repeat |
| | | | | | | proteins. |
| 1641 | BL00115X | 21.13 | 5.500e−35 | 1324-1359 | BL00115 | Eukaryotic RNA |
| | | | | | | polymerase II |
| | | | | | | heptapeptide repeat |
| | | | | | | proteins. |
| 1641 | BL00115Y | 11.86 | 1.000e−40 | 1360-1405 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−08 | 1568-1616 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1604-1652 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1611-1659 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1618-1666 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1625-1673 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1632-1680 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1639-1687 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1646-1694 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1653-1701 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1660-1708 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1667-1715 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1674-1722 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1681-1729 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1688-1736 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1695-1743 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1702-1750 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1709-1757 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1716-1764 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1723-1771 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1730-1778 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1737-1785 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1744-1792 | BL00115 | Eukaryotic RNA |
| | | | | | | polymerase II |
| | | | | | | heptapeptide repeat |
| | | | | | | proteins |
| 1641 | BL00115Z | 3.12 | 1.000e−40 | 1751-1799 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1758-1806 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1765-1813 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1772-1820 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1779-1827 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1786-1834 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1793-1841 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1800-1848 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1814-1862 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1821-1869 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1828-1876 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1835-1883 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1842-1890 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1849-1897 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1856-1904 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1863-1911 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1870-1918 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1877-1925 | BL00115 | Eukaryotic RNA |
| | | | | | | polymerase II |
| | | | | | | heptapeptide repeat |
| | | | | | | proteins. |
| 1641 | BL00115Z | 3.12 | 1.000e−40 | 1884-1932 | BL00015 | Eukaryotic RNA |
| | | | | | | polymerase II |
| | | | | | | heptapeptide repeat |
| | | | | | | proteins. |
| 1641 | BL00115Z | 3.12 | 1.000e−40 | 1891-1939 | BL00115 | Eukaryotic RNA |
| | | | | | | polymerase II |
| | | | | | | heptapeptide repeat |
| | | | | | | proteins. |
| 1641 | BL00115Z | 3.12 | 1.493e−35 | 1597-1645 | BL00115 | Eukaryotic RNA |
| | | | | | | polymerase II |
| | | | | | | heptapeptide repeat |
| | | | | | | proteins. |
| 1641 | BL00115Z | 3.12 | 1.779e−17 | 1922-1970 | BL00115 | Eukaryotic RNA |
| | | | | | | polymerase II |
| | | | | | | heptapeptide repeat |
| | | | | | | proteins. |
| 1641 | BL00115Z | 3.12 | 2.636e−12 | 1561-1609 | BL00115 | Eukaryotic RNA |
| | | | | | | polymerase II |
| | | | | | | heptapeptide repeat |
| | | | | | | proteins. |
| 1641 | BL00115Z | 3.12 | 3.326e−24 | 1590-1638 | BL00115 | Eukaryotic RNA |
| | | | | | | polymerase II |
| | | | | | | heptapeptide repeat |
| | | | | | | proteins. |
| 1641 | BL00115Z | 3.12 | 3.380e−12 | 1554-1602 | BL00115 | Eukaryotic RNA |
| | | | | | | polymerase II |
| | | | | | | heptapeptide repeat |
| | | | | | | proteins. |
| 1641 | BL00115Z | 3.12 | 3.512e−10 | 1576-1624 | BL00115 | Eukaryotic RNA |
| | | | | | | polymerase II |
| | | | | | | heptapeptide repeat |
| | | | | | | proteins. |
| 1641 | BL00115Z | 3.12 | 4.030e−18 | 1583-1631 | BL00115 | Eukaryotic RNA |
| | | | | | | polymerase II |
| | | | | | | heptapeptide repeat |
| | | | | | | proteins. |
| 1641 | BL00115Z | 3.12 | 4.646e−30 | 1905-1953 | BL00115 | Eukaryotic RNA |
| | | | | | | polymerase II |
| | | | | | | heptapeptide repeat |
| | | | | | | proteins. |
| 1641 | BL00115Z | 3.12 | 5.760e−17 | 1908-1956 | BL00115 | Eukaryotic RNA |
| | | | | | | polymerase II |
| | | | | | | heptapeptide repeat |
| | | | | | | proteins. |
| 1641 | BL00115Z | 3.12 | 6.000e−23 | 1912-1960 | BL00115 | Eukaryotic RNA |
| | | | | | | polymerase II |
| | | | | | | heptapeptide repeat |
| | | | | | | proteins. |
| 1641 | BL00115Z | 3.12 | 6.302e−10 | 1894-1942 | BL00115 | Eukaryotic RNA |
| | | | | | | polymerase II |
| | | | | | | heptapeptide repeat |
| | | | | | | proteins. |
| 1641 | BL00115Z | 3.12 | 6.591e−40 | 1807-1855 | BL00115 | Eukaryotic RNA |
| | | | | | | polymerase II |
| | | | | | | heptapeptide repeat |
| | | | | | | proteins. |
| 1641 | BL00115Z | 3.12 | 7.154e−09 | 1547-1595 | BL00115 | Eukaryotic RNA |
| | | | | | | polymerase II |
| | | | | | | heptapeptide repeat |
| | | | | | | proteins. |
| 1641 | BL00115Z | 3.12 | 8.174e−38 | 1898-1946 | BL00115 | Eukaryotic RNA |
| | | | | | | polymerase II |
| | | | | | | heptapeptide repeat |
| | | | | | | proteins. |
| 1641 | BL00115Z | 3.12 | 8.566e−14 | 1901-1949 | BL00115 | Eukaryotic RNA |
| | | | | | | polymerase II |
| | | | | | | heptapeptide repeat |
| | | | | | | proteins. |
| 1641 | BL00115Z | 3.12 | 9.644e−18 | 1919-1967 | BL00115 | Eukaryotic RNA |
| | | | | | | polymerase II |
| | | | | | | heptapeptide repeat |
| | | | | | | proteins. |
| 1641 | BL00115Z | 3.12 | 9.903e−22 | 1915-1963 | BL00115 | Eukaryotic RNA |
| | | | | | | polymerase II |
| | | | | | | heptapeptide repeat |
| | | | | | | proteins. |
| 1643 | BL00298A | 10.97 | 1.000e−40 | 74-118 | BL00298 | Heat shock hsp90 |
| | | | | | | proteins family |
| | | | | | | proteins. |
| 1643 | BL00298B | 15.64 | 1.290e−39 | 134-180 | BL00298 | Heat shock hsp90 |
| | | | | | | proteins family |
| | | | | | | proteins. |
| 1643 | BL00298C | 16.40 | 2.286e−40 | 186-229 | BL00298 | Heat shock hsp90 |
| | | | | | | proteins family |
| | | | | | | proteins. |
| 1643 | BL00298D | 17.97 | 6.226e−33 | 242-281 | BL00298 | Heat shock hsp90 |
| | | | | | | proteins family |
| | | | | | | proteins. |
| 1643 | BL00298E | 27.30 | 1.000e−40 | 321-375 | BL00298 | Heat shock hsp90 |
| | | | | | | proteins family |
| | | | | | | proteins. |
| 1643 | BL00298F | 11.21 | 1.000e−40 | 409-463 | BL00298 | Heat shock hsp90 |
| | | | | | | proteins family |
| | | | | | | proteins. |
| 1643 | BL00298G | 24.57 | 5.345e−39 | 465-519 | BL00298 | Heat shock hsp90 |
| | | | | | | proteins family |
| | | | | | | proteins. |
| 1643 | BL00298H | 20.50 | 1.000e−40 | 553-606 | BL00298 | Heat shock hsp90 |
| | | | | | | proteins family |
| | | | | | | proteins. |
| 1643 | BL00298I | 30.07 | 7.818e−34 | 661-714 | BL00298 | Heat shock hsp90 |
| | | | | | | proteins family |
| | | | | | | proteins. |
| 1643 | BL00422C | 16.18 | 9.234e−10 | 295-322 | BL00422 | Granins proteins. |
| 1643 | PD01781B | 27.55 | 2.800e−09 | 758-801 | PD01781 | PROTEASE IMMUNOGLOBULIN |
| | | | | | | PRECURSO. |
| 1643 | PR00775A | 9.90 | 3.250e−22 | 74-94 | PR00775 | 90 KD HEAT SHOCK |
| | | | | | | PROTEIN SIGNATURE |
| 1643 | PR00775B | 3.52 | 3.700e−26 | 95-117 | PR00775 | 90 KD HEAT SHOCK |
| | | | | | | PROTEIN SIGNATURE |
| 1643 | PR00775C | 10.68 | 3.727e−16 | 144-161 | PR00775 | 90 KD HEAT SHOCK |
| | | | | | | PROTEIN SIGNATURE |
| 1643 | PR00775D | 8.91 | 9.640e−18 | 162-179 | PR00775 | 90 KD HEAT SHOCK |
| | | | | | | PROTEIN SIGNATURE |
| 1643 | PR00775E | 8.06 | 6.571e−27 | 192-214 | PR00775 | 90 KD HEAT SHOCK |
| | | | | | | PROTEIN SIGNATURE |
| 1643 | PR00775F | 12.76 | 2.800e−15 | 243-260 | PR00775 | 90 KD HEAT SHOCK |
| | | | | | | PROTEIN SIGNATURE |
| 1643 | PR00775G | 10.64 | 9.625e−19 | 261-279 | PR00775 | 90 KD HEAT SHOCK |
| | | | | | | PROTEIN SIGNATURE |
| 1644 | BL00299 | 28.84 | 1.000e−40 | 16-67 | BL00299 | Ubiquitin domain |
| | | | | | | proteins. |
| 1644 | PF01020 | 15.00 | 1.000e−40 | 80-128 | PF01020 | Ribosomal L40e family. |
| 1644 | PR00348A | 7.86 | 2.000e−26 | 11-31 | PR00348 | UBIQUITIN SIGNATURE |
| 1644 | PR00348B | 5.78 | 2.800e−27 | 32-52 | PR00348 | UBIQUITIN SIGNATURE |
| 1644 | PR00348C | 20.03 | 1.643e−27 | 53-74 | PR00348 | UBIQUITIN SIGNATURE |
| 1646 | BL00646A | 25.82 | 6.192e−29 | 14-61 | BL00646 | Ribosomal protein S13 |
| | | | | | | proteins. |
| 1646 | BL00646B | 21.42 | 6.100e−30 | 110-142 | BL00646 | Ribosomal protein S13 |
| | | | | | | proteins. |
| 1647 | BL00358A | 13.06 | 9.690e−11 | 19-29 | BL00358 | Ribosomal protein L5 |
| | | | | | | proteins. |
| 1647 | BL00358B | 22.76 | 2.742e−38 | 40-85 | BL00358 | Ribosomal protein L5 |
| | | | | | | proteins. |
| 1647 | BL00358C | 13.75 | 5.500e−15 | 99-112 | BL00358 | Ribosomal protein L5 |
| | | | | | | proteins. |
| 1647 | BL00358D | 14.26 | 5.500e−14 | 119-133 | BL00358 | Ribosomal protein L5 |
| | | | | | | proteins. |
| 1649 | BL00290A | 20.89 | 7.158e−13 | 42-64 | BL00290 | Immunoglobulins and |
| | | | | | | major |
| | | | | | | histocompatibility |
| | | | | | | complex proteins. |
| 1649 | BL00290B | 13.17 | 4.500e−12 | 98-115 | BL00290 | Immunoglobulins and |
| | | | | | | major |
| | | | | | | histocompatibility |
| | | | | | | complex proteins. |
| 1650 | BL01121A | 9.11 | 9.100e−12 | 90-100 | BL01121 | Caspase family |
| | | | | | | histidine proteins. |
| 1650 | BL01121B | 29.68 | 1.346e−26 | 103-138 | BL01121 | Caspase family |
| | | | | | | histidine proteins. |
| 1650 | BL01121C | 13.17 | 5.065e−15 | 150-165 | BL01121 | Caspase family |
| | | | | | | histidine proteins. |
| 1650 | BL01121D | 12.34 | 4.343e−19 | 197-214 | BL01121 | Caspase family |
| | | | | | | histidine proteins. |
| 1650 | BL01121E | 23.71 | 5.179e−31 | 247-281 | BL01121 | Caspase family |
| | | | | | | histidine proteins. |
| 1650 | BL01121F | 15.43 | 1.783e−12 | 293-305 | BL01121 | Caspase family |
| | | | | | | histidine proteins. |
| 1650 | PR00376A | 14.23 | 5.886e−14 | 88-101 | PR00376 | INTERLEUKIN-1B |
| | | | | | | CONVERTING ENZYME |
| | | | | | | SIGNATURE |
| 1650 | PR00376B | 15.57 | 9.660e−11 | 106-124 | PR00376 | INTERLEUKIN-1B |
| | | | | | | CONVERTING ENZYME |
| | | | | | | SIGNATURE |
| 1650 | PR00376C | 16.28 | 5.574e−10 | 124-142 | PR00376 | INTERLEUKIN-1B |
| | | | | | | CONVERTING ENZYME |
| | | | | | | SIGNATURE |
| 1650 | PR00376D | 12.74 | 1.250e−10 | 157-165 | PR00376 | INTERLEUKIN-1B |
| | | | | | | CONVERTING ENZYME |
| | | | | | | SIGNATURE |
| 1650 | PR00376E | 12.26 | 5.941e−18 | 197-215 | PR00376 | INTERLEUKIN-1B |
| | | | | | | CONVERTING ENZYME |
| | | | | | | SIGNATURE |
| 1650 | PR00376F | 13.77 | 4.833e−11 | 266-277 | PR00376 | INTERLEUKIN-1B |
| | | | | | | CONVERTING ENZYME |
| | | | | | | SIGNATURE |
| 1651 | BL00525A | 11.57 | 4.971e−11 | 12-30 | BL00525 | Ribosomal protein L6 |
| | | | | | | proteins. |
| 1651 | BL00700A | 19.34 | 9.308e−33 | 8-45 | BL00700 | Ribosonal protein L6 |
| | | | | | | proteins 2. |
| 1651 | BL00700B | 18.26 | 1.000e−40 | 62-100 | BL00700 | Ribosomal protein L6 |
| | | | | | | proteins 2. |
| 1651 | BL00700C | 11.76 | 4.000e−13 | 112-121 | BL00700 | Ribosomal protein L6 |
| | | | | | | proteins 2. |
| 1651 | BL00700D | 21.51 | 1.000e−40 | 141-184 | BL00700 | Ribosomal protein L6 |
| | | | | | | proteins 2. |
| 1652 | BL00055 | 15.98 | 2.200e−24 | 90-125 | BL00055 | Ribosomal protein S12 |
| | | | | | | proteins. |
| 1653 | BL00299 | 28.84 | 8.269e−33 | 16-67 | BL00299 | Ubiquitin domain |
| | | | | | | proteins. |
| 1653 | PR00348B | 5.78 | 2.000e−15 | 32-52 | PR00348 | UBIQUITIN SIGNATURE |
| 1653 | PR00348C | 20.03 | 8.477e−14 | 53-74 | PR00348 | UBIQUITIN SIGNATURE |
| 1654 | BL00024A | 11.49 | 5.000e−11 | 75-85 | BL00024 | Hemopexin domain |
| | | | | | | proteins. |
| 1654 | BL00024B | 21.53 | 3.432e−26 | 104-137 | BL00024 | Hemopexin domain |
| | | | | | | proteins. |
| 1654 | BL00024C | 22.98 | 1.000e−40 | 152-200 | BL00024 | Hemopexin domain |
| | | | | | | proteins. |
| 1654 | BL00024D | 17.28 | 4.375e−29 | 206-237 | BL00024 | Hemopexin domain |
| | | | | | | proteins. |
| 1654 | BL00024E | 7.58 | 2.406e−14 | 245-258 | BL00024 | Hemopexin domain |
| | | | | | | proteins. |
| 1654 | BL00024F | 11.30 | 6.786e−21 | 298-318 | BL00024 | Hemopexin domain |
| | | | | | | proteins. |
| 1654 | BL00024G | 13.31 | 2.333e−09 | 429-441 | BL00024 | Hemopexin domain |
| | | | | | | proteins. |
| 1654 | BL00024G | 13.31 | 9.308e−13 | 336-348 | BL00024 | Hemopexin domain |
| | | | | | | proteins. |
| 1654 | BL00024H | 11.35 | 2.174e−13 | 355-366 | BL00024 | Hemopexin domain |
| | | | | | | proteins. |
| 1654 | BL00024H | 11.35 | 3.600e−09 | 401-412 | BL00024 | Hemopexin domain |
| | | | | | | proteins. |
| 1654 | BL00142 | 8.38 | 2.636e−11 | 212-222 | BL00142 | Neutral zinc |
| | | | | | | metallopeptidases, |
| | | | | | | zinc-binding region |
| | | | | | | proteins. |
| 1654 | BL00546A | 19.62 | 8.154e−21 | 55-84 | BL00546 | Matrixins cysteine |
| | | | | | | switch. |
| 1654 | BL00546B | 20.11 | 1.000e−40 | 153-196 | BL00546 | Matrixins cysteine |
| | | | | | | switch. |
| 1654 | BL00546C | 16.41 | 2.575e−29 | 206-237 | BL00546 | Matrixins cysteine |
| | | | | | | switch. |
| 1654 | BL00546D | 10.34 | 2.459e−14 | 245-258 | BL00546 | Matrixins cysteine |
| | | | | | | switch. |
| 1654 | BL00546E | 10.23 | 9.727e−21 | 298-318 | BL00546 | Matrixins cysteine |
| | | | | | | switch. |
| 1654 | BL00546F | 12.40 | 6.700e−13 | 336-348 | BL00546 | Matrixins cysteine |
| | | | | | | switch. |
| 1654 | BL00546F | 12.40 | 8.059e−10 | 429-441 | BL00546 | Matrixins cysteine |
| | | | | | | switch. |
| 1654 | BL00546G | 16.84 | 2.091e−15 | 356-375 | BL00546 | Matrixins cysteine |
| | | | | | | switch. |
| 1654 | BL00546G | 16.84 | 3.368e−09 | 310-329 | BL00546 | Matrixins cysteine |
| | | | | | | switch. |
| 1654 | BL00546H | 10.76 | 1.514e−10 | 448-458 | BL00546 | Matrixins cysteine |
| | | | | | | switch. |
| 1654 | BL00546H | 10.76 | 6.333e−11 | 401-411 | BL00546 | Matrixins cysteine |
| | | | | | | switch. |
| 1654 | PR00138A | 15.14 | 7.000e−13 | 75-88 | PR00138 | MATRIXIN SIGNATURE |
| 1654 | PR00138B | 15.82 | 6.625e−16 | 130-145 | PR00138 | MATRIXIN SIGNATURE |
| 1654 | PR00138C | 16.41 | 9.053e−33 | 153-181 | PR00138 | MATRIXIN SIGNATURE |
| 1654 | PR00138D | 16.56 | 6.921e−25 | 212-237 | PR00138 | MATRIXIN SIGNATURE |
| 1654 | PR00138E | 6.01 | 2.091e−14 | 245-258 | PR00138 | MATRIXIN SIGNATURE |
| 1654 | PR00480B | 15.41 | 5.355e−11 | 207-225 | PR00480 | ASTACIN FAMILY |
| | | | | | | SIGNATURE |
| 1656 | PR00449E | 13.50 | 8.313e−18 | 68-90 | PR00449 | TRANSFORMING PROTEIN |
| | | | | | | P21 RAS SIGNATURE |
| 1657 | BL01160B | 19.54 | 2.068e−09 | 139-192 | BL01160 | Kinesin light chain |
| | | | | | | repeat proteins. |
| 1657 | PF00850D | 14.76 | 5.846e−16 | 829-852 | PF00850 | Histone deacetylase |
| | | | | | | family. |
| 1657 | PF00850E | 8.88 | 8.071e−23 | 863-888 | PF00850 | Histone deacetylase |
| | | | | | | family. |
| 1657 | PF00850F | 15.70 | 1.556e−10 | 901-933 | PF00850 | Histone deacetylase |
| | | | | | | family. |
| 1657 | PF00850G | 22.75 | 9.250e−11 | 940-981 | PF00850 | Histone deacetylase |
| | | | | | | family. |
| 1658 | BL00720B | 16.57 | 6.516e−17 | 347-370 | BL00720 | Guanine-nucleotide |
| | | | | | | dissociation |
| | | | | | | stimulators CDC25 |
| | | | | | | family sign. |
| 1658 | PR00761E | 14.32 | 9.500e−10 | 38-56 | PR00761 | BINDIN PRECURSOR |
| | | | | | | SIGNATURE |
| 1659 | BL01267A | 18.07 | 7.618e−28 | 12-40 | BL01267 | Uncharacterized protein |
| | | | | | | family UPF0023 |
| | | | | | | proteins. |
| 1659 | BL01267B | 18.76 | 6.936e−31 | 41-81 | BL01267 | Uncharacterized protein |
| | | | | | | family UPF0023 |
| | | | | | | proteins. |
| 1659 | BL01267C | 22.80 | 9.348e−23 | 125-178 | BL01267 | Uncharacterized protein |
| | | | | | | family UPF0023 |
| | | | | | | proteins. |
| 1660 | BL01100A | 12.62 | 1.947e−24 | 12-38 | BL01100 | NNMT/PNMT/TEMT family |
| | | | | | | of methyltransferases |
| | | | | | | proteins. |
| 1660 | BL01100B | 12.73 | 1.000e−40 | 42-85 | BL01100 | NNMT/PNMT/TEMT family |
| | | | | | | of methyltransferases |
| | | | | | | proteins. |
| 1660 | BL01100C | 10.34 | 6.211e−24 | 86-107 | BL01100 | NNMT/PNMT/TEMT family |
| | | | | | | of methyltransferases |
| | | | | | | proteins. |
| 1660 | BL01100D | 14.77 | 9.357e−23 | 110-134 | BL01100 | NNMT/PNMT/TEMT family |
| | | | | | | of methyltransferases |
| | | | | | | proteins. |
| 1660 | BL01100E | 12.25 | 1.000e−40 | 156-199 | BL01100 | NNMT/PNMT/TEMT family |
| | | | | | | of methyltransferases |
| | | | | | | proteins. |
| 1660 | BL01100F | 14.30 | 6.595e−28 | 200-232 | BL01100 | NNMT/PNMT/TEMT family |
| | | | | | | of methyltransferases |
| | | | | | | proteins. |
| 1660 | BL01100G | 14.70 | 9.780e−19 | 238-259 | BL01100 | NNMT/PNMT/TEMT family |
| | | | | | | of methyltransferases |
| | | | | | | proteins. |
| 1661 | BL00548 | 20.58 | 7.000e−19 | 66-95 | BL00548 | Ribosomal protein S3 |
| | | | | | | proteins. |
| 1661 | BL00765A | 13.27 | 1.000e−08 | 158-178 | BL00765 | Phosphoglucose |
| | | | | | | isomerase proteins. |
| 1661 | PF00013 | 5.78 | 4.706e−10 | 58-69 | PF00013 | KH domain proteins |
| | | | | | | family of RNA binding |
| | | | | | | proteins. |
| 1662 | PD01733B | 20.44 | 4.375e−09 | 410-464 | PD01733 | APOLIPOPROTEIN PLASMA |
| | | | | | | LIPID TRANSPORT H. |
| 1663 | BL00267 | 14.21 | 8.667e−10 | 81-90 | BL00267 | Tachykinin family |
| | | | | | | proteins. |
| 1664 | DM00372A | 19.18 | 1.000e−40 | 19-63 | DM00372 | CARCINOEMBRYONIC |
| | | | | | | ANTIGEN PRECURSOR |
| | | | | | | AMINO-TERMINAL DOMAIN. |
| 1664 | DM00372B | 20.31 | 9.229e−38 | 82-126 | DM00372 | CARCINOEMBRYONIC |
| | | | | | | ANTIGEN PRECURSOR |
| | | | | | | AMINO-TERMINAL DOMAIN. |
| 1664 | DM00372C | 23.69 | 2.552e−28 | 321-356 | DM00372 | CARCINOEMBRYONIC |
| | | | | | | ANTIGEN PRECURSOR |
| | | | | | | AMINO-TERMINAL DOMAIN. |
| 1664 | PD01270D | 24.66 | 1.000e−09 | 378-413 | PD01270 | RECEPTOR FC |
| | | | | | | IMMUNOGLOBULIN AFFIN. |
| 1665 | DM01269A | 23.35 | 1.600e−20 | 68-95 | DM01269 | 303 kw ACTIVATING RAN |
| | | | | | | GTPASE ISOZYME. |
| 1665 | DM01269B | 11.71 | 3.323e−09 | 138-147 | DM01269 | 303 kw ACTIVATING RAN |
| | | | | | | GTPASE ISOZYME. |
| 1665 | PF00638 | 11.91 | 4.600e−18 | 67-81 | PF00638 | RanBP1 domain proteins. |
| 1666 | BL00633B | 13.82 | 7.097e−16 | 166-190 | BL00633 | Bromodomain proteins. |
| 1666 | PR00503B | 9.96 | 9.571e−13 | 165-181 | PR00503 | BROMODOMAIN SIGNATURE |
| 1666 | PR00503C | 19.84 | 7.563e−09 | 181-199 | PR00503 | BROMODOMAIN SIGNATURE |
| 1666 | PR00503D | 20.81 | 2.313e−11 | 199-218 | PR00503 | BROMODOMAIN SIGNATURE |
| 1670 | BL00282 | 16.88 | 8.875e−12 | 464-486 | BL00282 | Kazal serine protease |
| | | | | | | inhibitors family |
| | | | | | | proteins. |
| 1670 | BL00612A | 12.09 | 3.136e−11 | 3-18 | BL00612 | Osteonectin domain |
| | | | | | | proteins. |
| 1670 | BL00612B | 11.35 | 6.625e−29 | 431-463 | BL00612 | Osteonectin domain |
| | | | | | | proteins. |
| 1670 | BL00612C | 9.90 | 1.000e−40 | 496-543 | BL00612 | Osteonectin domain |
| | | | | | | proteins. |
| 1670 | BL00612D | 10.06 | 1.000e−40 | 563-613 | BL00612 | Osteonectin domain |
| | | | | | | proteins. |
| 1670 | BL00612E | 13.12 | 1.000e−40 | 617-661 | BL00612 | Osteonectin domain |
| | | | | | | proteins. |
| 1671 | BL00061A | 9.41 | 6.684e−11 | 83-93 | BL00061 | Short-chain |
| | | | | | | dehydrogenases/reductases |
| | | | | | | family proteins. |
| 1671 | BL00061B | 25.79 | 7.158e−19 | 136-173 | BL00061 | Short-chain |
| | | | | | | dehydrogenases/reductases |
| | | | | | | family proteins. |
| 1671 | PR00080A | 9.32 | 9.667e−11 | 83-94 | PR00080 | ALCOHOL DEHYDROGENASE |
| | | | | | | SUPERFAMILY SIGNATURE |
| 1671 | PR00080C | 17.16 | 2.125e−10 | 156-175 | PR00080 | ALCOHOL DEHYDROGENASE |
| | | | | | | SUPERFAMILY SIGNATURE |
| 1671 | PR00081A | 10.53 | 7.000e−12 | 5-22 | PR00081 | GLUCOSE/RIBITOL |
| | | | | | | DEHYDROGENASE FAMILY |
| | | | | | | SIGNATURE |
| 1671 | PR00081B | 10.38 | 3.368e−10 | 83-94 | PR00081 | GLUCOSE/RIBITOL |
| | | | | | | DEHYDROGENASE FAMILY |
| | | | | | | SIGNATURE |
| 1671 | PR00081C | 15.13 | 6.586e−10 | 130-146 | PR00081 | GLUCOSE/RIBITOL |
| | | | | | | DEHYDROGENASE FAMILY |
| | | | | | | SIGNATURE |
| 1672 | BL00139A | 10.29 | 1.000e−14 | 132-141 | BL00139 | Eukaryotic thiol |
| | | | | | | (cysteine) proteases |
| | | | | | | cysteine proteins. |
| 1672 | BL00139B | 10.19 | 6.571e−09 | 175-183 | BL00139 | Eukaryotic thiol |
| | | | | | | (cysteine) proteases |
| | | | | | | cysteine proteins. |
| 1672 | BL00139C | 9.23 | 2.800e−10 | 275-284 | BL00139 | Eukaryotic thiol |
| | | | | | | (cysteine) proteases |
| | | | | | | cysteine proteins. |
| 1672 | BL00139D | 9.24 | 1.818e−18 | 295-311 | BL00139 | Eukaryotic thiol |
| | | | | | | (cysteine) proteases |
| | | | | | | cysteine proteins. |
| 1672 | PD01919E | 19.31 | 7.582e−10 | 73-98 | PD01919 | PROTEIN PROCESSING |
| | | | | | | TRANSPORT CAPSID |
| | | | | | | ASSEMBLY PR. |
| 1672 | PR00704C | 11.88 | 6.162e−09 | 132-148 | PR00704 | CALPAIN CYSTEINE |
| | | | | | | PROTEASE (C2) FAMILY |
| | | | | | | SIGNATURE |
| 1672 | PR00705A | 10.55 | 4.000e−21 | 132-147 | PR00705 | PAPAIN CYSTEINE |
| | | | | | | PROTEASE (C1) FAMILY |
| | | | | | | SIGNATURE |
| 1672 | PR00705B | 10.22 | 2.385e−10 | 276-286 | PR00705 | PAPAIN CYSTEINE |
| | | | | | | PROTEASE (C1) FAMILY |
| | | | | | | SIGNATURE |
| 1673 | BL00610A | 17.73 | 1.000e−40 | 60-109 | BL00610 | Sodium: neurotransmitter |
| | | | | | | symporter family |
| | | | | | | proteins. |
| 1673 | BL00610B | 23.65 | 1.000e−40 | 123-172 | BL00610 | Sodium: neurotransmitter |
| | | | | | | symporter family |
| | | | | | | proteins. |
| 1673 | BL00610C | 12.94 | 1.000e−40 | 233-284 | BL00610 | Sodium: neurotransmitter |
| | | | | | | symporter family |
| | | | | | | proteins. |
| 1673 | BL00610D | 20.97 | 1.000e−40 | 299-351 | BL00610 | Sodium: neurotransmitter |
| | | | | | | symporter family |
| | | | | | | proteins. |
| 1673 | BL00610E | 20.34 | 3.423e−39 | 392-434 | BL00610 | Sodium: neurotransmitter |
| | | | | | | symporter family |
| | | | | | | proteins. |
| 1673 | BL00610F | 29.02 | 1.000e−40 | 491-545 | BL00610 | Sodium: neurotransmitter |
| | | | | | | symporter family |
| | | | | | | proteins. |
| 1673 | BL00610G | 12.89 | 4.706e−25 | 551-573 | BL00610 | Sodium: neurotransmitter |
| | | | | | | symporter family |
| | | | | | | proteins. |
| 1673 | PR00176A | 16.82 | 8.500e−28 | 60-81 | PR00176 | SODIUM/NEUROTRANSMITTER |
| | | | | | | SYMPORTER SIGNATURE |
| 1673 | PR00176B | 7.31 | 7.250e−18 | 89-108 | PR00176 | SODIUM/NEUROTRANSMITTER |
| | | | | | | SYMPORTER SIGNATURE |
| 1673 | PR00176C | 10.84 | 8.714e−27 | 132-158 | PR00176 | SODIUM/NEUROTRANSMITTER |
| | | | | | | SYMPORTER SIGNATURE |
| 1673 | PR00176D | 9.02 | 1.783e−19 | 260-277 | PR00176 | SODIUM/NEUROTRANSMITTER |
| | | | | | | SYMPORTER SIGNATURE |
| 1673 | PR00176E | 11.41 | 7.000e−21 | 342-362 | PR00176 | SODIUM/NEUROTRANSMITTER |
| | | | | | | SYMPORTER SIGNATURE |
| 1673 | PR00176F | 10.73 | 2.333e−20 | 396-415 | PR00176 | SODIUM/NEUROTRANSMITTER |
| | | | | | | SYMPORTER SIGNATURE |
| 1673 | PR00176G | 12.48 | 7.857e−20 | 480-500 | PR00176 | SODIUM/NEUROTRANSMITTER |
| | | | | | | SYMPORTER SIGNATURE |
| 1673 | PR00176H | 15.27 | 5.500e−21 | 520-540 | PR00176 | SODIUM/NEUROTRANSMITTER |
| | | | | | | SYMPORTER SIGNATURE |
| 1674 | BL00309C | 18.65 | 1.621e−09 | 62-86 | BL00309 | Vertebrate galactoside- |
| | | | | | | binding lectin |
| | | | | | | proteins. |
| 1675 | BL00113A | 12.74 | 9.585e−09 | 157-173 | BL00113 | Adenylate kinase |
| | | | | | | proteins. |
| 1675 | BL00690A | 6.87 | 3.455e−12 | 160-169 | BL00690 | DEAH-box subfamily ATP- |
| | | | | | | dependent helicases |
| | | | | | | proteins. |
| 1675 | BL00690B | 13.38 | 4.938e−18 | 191-208 | BL00690 | DEAH-box subfamily ATP- |
| | | | | | | dependent helicases |
| | | | | | | proteins. |
| 1675 | BL00690C | 7.51 | 8.650e−11 | 257-266 | BL00690 | DEAH-box subfamily ATP- |
| | | | | | | dependent helicases |
| | | | | | | proteins. |
| 1675 | DM00406 | 7.73 | 9.514e−10 | 392-404 | DM00406 | GLIADIN. |
| 1676 | BL01082 | 20.37 | 3.500e−09 | 47-86 | BL01082 | Ribosomal protein L7Ae |
| | | | | | | proteins. |
| 1676 | BL01189A | 14.27 | 1.000e−40 | 35-70 | BL01189 | Ribosomal protein S12e |
| | | | | | | proteins. |
| 1676 | BL01189B | 13.49 | 1.000e−40 | 71-124 | BL01189 | Ribosomal protein S12e |
| | | | | | | proteins. |
| 1676 | PR00972A | 8.01 | 6.625e−17 | 21-34 | PR00972 | RIBOSOMAL PROTEIN S12E |
| | | | | | | FAMILY SIGNATURE |
| 1676 | PR00972B | 8.62 | 9.000e−21 | 35-51 | PR00972 | RIBOSOMAL PROTEIN S12E |
| | | | | | | FAMILY SIGNATURE |
| 1676 | PR00972C | 10.31 | 1.500e−26 | 76-97 | PR00972 | RIBOSOMAL PROTEIN S12E |
| | | | | | | FAMILY SIGNATURE |
| 1676 | PR00972D | 10.15 | 1.000e−18 | 101-114 | PR00972 | RIBOSOMAL PROTEIN S12E |
| | | | | | | FAMILY SIGNATURE |
| 1677 | PF00043 | 21.83 | 3.647e−22 | 54-83 | PF00043 | Glutathione S- |
| | | | | | | transferases. |
| 1677 | PR00455B | 17.47 | 5.227e−09 | 60-83 | PR00455 | TETR BACTERIAL |
| | | | | | | REGULATORY PROTEIN HTH |
| | | | | | | SIGNATURE |
| 1678 | BL00501A | 21.02 | 7.333e−18 | 106-131 | BL00501 | Signal peptidases I |
| | | | | | | serine proteins. |
| 1678 | BL00501B | 12.58 | 9.735e−11 | 132-147 | BL00501 | Signal peptidases I |
| | | | | | | serine proteins. |
| 1678 | BL00501C | 9.61 | 4.000e−10 | 156-167 | BL00501 | Signal peptidases I |
| | | | | | | serine proteins. |
| 1678 | BL00501D | 16.69 | 1.000e−15 | 179-198 | BL00501 | Signal peptidases I |
| | | | | | | serine proteins. |
| 1678 | PR00728A | 13.14 | 1.750e−20 | 125-140 | PR00728 | EUKARYOTIC SIGNAL |
| | | | | | | PEPTIDASE (S27) FAMILY |
| | | | | | | SIGNATURE |
| 1678 | PR00728B | 11.12 | 1.000e−15 | 184-195 | PR00728 | EUKARYOTIC SIGNAL |
| | | | | | | PEPTIDASE (S27) FAMILY |
| | | | | | | SIGNATURE |
| 1683 | BL01019A | 13.20 | 3.859e−10 | 57-96 | BL01019 | ADP-ribosylation |
| | | | | | | factors family |
| | | | | | | proteins. |
| 1683 | BL01115A | 10.22 | 9.735e−12 | 23-66 | BL01115 | GTP-binding nuclear |
| | | | | | | protein ran proteins. |
| 1683 | PR00449A | 13.20 | 3.400e−15 | 23-44 | PR00449 | TRANSFORMING PROTEIN |
| | | | | | | P21 RAS SIGNATURE |
| 1683 | PR00449B | 14.34 | 2.800e−10 | 46-62 | PR00449 | TRANSFORMING PROTEIN |
| | | | | | | P21 RAS SIGNATURE |
| 1683 | PR00449C | 17.27 | 8.941e−19 | 64-86 | PR00449 | TRANSFORMING PROTEIN |
| | | | | | | P21 RAS SIGNATURE |
| 1683 | PR00449D | 10.79 | 3.864e−12 | 126-139 | PR00449 | TRANSFORMING PROTEIN |
| | | | | | | P21 RAS SIGNATURE |
| 1683 | PR00449E | 13.50 | 6.478e−15 | 161-183 | PR00449 | TRANSFORMING PROTEIN |
| | | | | | | P21 RAS SIGNATURE |
| 1684 | BL00604A | 9.13 | 8.842e−20 | 49-103 | BL00604 | Synaptophysin/ |
| | | | | | | synaptoporin proteins. |
| 1684 | BL00604B | 9.95 | 1.329e−19 | 114-143 | BL00604 | Synaptophysin/ |
| | | | | | | synaptoporin proteins. |
| 1684 | BL00604C | 14.66 | 5.639e−12 | 144-175 | BL00604 | Synaptophysin/ |
| | | | | | | synaptoporin proteins. |
| 1684 | BL00604D | 12.28 | 5.410e−11 | 176-210 | BL00604 | Synaptophysin/ |
| | | | | | | synaptoporin proteins. |
| 1684 | BL00604E | 8.32 | 1.444e−23 | 210-251 | BL00604 | Synaptophysin/ |
| | | | | | | synaptoporin proteins. |
| 1684 | PR00220A | 10.93 | 8.244e−24 | 46-68 | PR00220 | SYNAPTOPHYSIN/SYNAPTOPORIN |
| | | | | | | FAMILY SIGNATURE |
| 1684 | PR00220B | 15.48 | 4.462e−24 | 70-95 | PR00220 | SYNAPTOPHYSIN/SYNAPTOPORIN |
| | | | | | | FAMILY SIGNATURE |
| 1684 | PR00220C | 11.05 | 4.477e−25 | 127-151 | PR00220 | SYNAPTOPHYSIN/SYNAPTOPORIN |
| | | | | | | FAMILY SIGNATURE |
| 1684 | PR00220D | 8.32 | 7.585e−26 | 159-182 | PR00220 | SYNAPTOPHYSIN/SYNAPTOPORIN |
| | | | | | | FAMILY SIGNATURE |
| 1684 | PR00220E | 3.46 | 6.932e−23 | 225-243 | PR00220 | SYNAPTOPHYSIN/SYNAPTOPORIN |
| | | | | | | FAMILY SIGNATURE |
| 1685 | BL00142 | 8.38 | 5.909e−11 | 352-362 | BL00142 | Neutral zinc |
| | | | | | | metallopeptidases, |
| | | | | | | zinc-binding region |
| | | | | | | proteins. |
| 1685 | BL00427 | 13.93 | 6.918e−24 | 449-503 | BL00427 | Disintegrins proteins. |
| 1685 | PR00289A | 13.62 | 5.000e−19 | 463-482 | PR00289 | DISINTEGRIN SIGNATURE |
| 1685 | PR00289B | 11.79 | 2.500e−12 | 492-504 | PR00289 | DISINTEGRIN SIGNATURE |
| 1685 | PR00480B | 15.41 | 8.500e−10 | 347-365 | PR00480 | ASTACIN FAMILY |
| | | | | | | SIGNATURE |
| 1686 | BL00421A | 11.79 | 4.600e−15 | 67-85 | BL00421 | Transmembrane 4 family |
| | | | | | | proteins. |
| 1686 | BL00421B | 17.62 | 9.000e−34 | 118-156 | BL00421 | Transmembrane 4 family |
| | | | | | | proteins. |
| 1686 | BL00421C | 12.89 | 2.333e−11 | 203-214 | BL00421 | Transmembrane 4 family |
| | | | | | | proteins. |
| 1686 | BL00421E | 20.97 | 4.500e−21 | 287-316 | BL00421 | Transmembrane 4 family |
| | | | | | | proteins. |
| 1686 | PR00174A | 8.67 | 8.105e−09 | 71-93 | PR00174 | LACY PROTON/SUGAR |
| | | | | | | SYMPORTER SIGNATURE |
| 1686 | PR00259A | 9.27 | 2.742e−21 | 71-94 | PR00259 | TRANSMEMBRANE FOUR |
| | | | | | | FAMILY SIGNATURE |
| 1686 | PR00259B | 14.81 | 6.727e−23 | 112-138 | PR00259 | TRANSMEMBRANE FOUR |
| | | | | | | FAMILY SIGNATURE |
| 1686 | PR00259C | 16.40 | 1.265e−22 | 139-167 | PR00259 | TRANSMEMBRANE FOUR |
| | | | | | | FAMILY SIGNATURE |
| 1686 | PR00259D | 13.50 | 6.250e−23 | 290-316 | PR00259 | TRANSMEMBRANE FOUR |
| | | | | | | FAMILY SIGNATURE |
| 1688 | BL00291A | 4.49 | 5.345e−09 | 138-172 | BL00291 | Prion protein. |
| 1688 | BL00291A | 4.49 | 7.724e−09 | 132-166 | BL00291 | Prion protein. |
| 1688 | DM01724 | 8.14 | 4.553e−09 | 156-175 | DM01724 | kw ALLERGEN POLLEN CIM1 |
| | | | | | | HOL-LI. |
| 1689 | PD01861A | 14.06 | 4.836e−11 | 60-83 | PD01861 | PROTEIN NUCLEAR |
| | | | | | | RIBONUCLEOPROTEIN SMALL |
| | | | | | | MRNA RNA. |
| 1691 | BL00107A | 18.39 | 6.760e−13 | 280-310 | BL00107 | Protein kinases ATP- |
| | | | | | | binding region |
| | | | | | | proteins. |
| 1691 | BL00107B | 13.31 | 1.000e−14 | 344-359 | BL00107 | Protein kinases ATP- |
| | | | | | | binding region |
| | | | | | | proteins. |
| 1691 | DM00191A | 8.16 | 4.840e−09 | 663-675 | DM00191 | w SPAC8A4.04C |
| | | | | | | RESISTANCE SPAC8A4.05C |
| | | | | | | DAUNORUBICIN. |

### Example 6

### Prediction of Signal Peptide Sequences contained in the Polypeptides of the Invention

The signal peptide regions for a plurality of the polypeptides of the invention were predicted using the Neural Network SignalP V1.1 program (Nielsen et al, (1997) Int. J. Neural Syst. 8, 581-599 herein incorporated by reference). One of skill in the art will recognize that the actual cleavage site may be different than that predicted by the computer program. The following table depicts the polypeptides of the invention that are predicted to have an N-terminal signal peptide, and lists the output from the SignalP program:

TABLE 5

| SEQ ID NO: | Max_score | Mean_score | Cleavage_Position |
|---|---|---|---|
| 865 | 0.979 | 0.879 | 34 |
| 870 | 0.896 | 0.685 | 23 |
| 876 | 0.979 | 0.854 | 33 |
| 893 | 0.941 | 0.838 | 15 |
| 895 | 0.989 | 0.619 | 44 |
| 897 | 0.954 | 0.805 | 25 |
| 898 | 0.897 | 0.486 | 21 |
| 904 | 0.959 | 0.826 | 28 |
| 906 | 0.931 | 0.729 | 30 |
| 919 | 0.958 | 0.608 | 39 |
| 921 | 0.980 | 0.932 | 26 |
| 923 | 0.970 | 0.872 | 22 |
| 930 | 0.943 | 0.602 | 23 |
| 934 | 0.897 | 0.678 | 23 |
| 937 | 0.956 | 0.597 | 43 |
| 938 | 0.882 | 0.547 | 25 |
| 939 | 0.967 | 0.532 | 16 |
| 942 | 0.926 | 0.509 | 36 |
| 943 | 0.896 | 0.540 | 25 |
| 954 | 0.997 | 0.951 | 36 |
| 959 | 0.979 | 0.751 | 39 |
| 960 | 0.983 | 0.637 | 36 |
| 964 | 0.964 | 0.819 | 26 |
| 968 | 0.943 | 0.645 | 38 |
| 969 | 0.969 | 0.597 | 30 |
| 970 | 0.958 | 0.821 | 30 |
| 973 | 0.945 | 0.587 | 37 |
| 974 | 0.910 | 0.623 | 25 |
| 988 | 0.991 | 0.971 | 27 |
| 994 | 0.914 | 0.628 | 26 |
| 999 | 0.970 | 0.659 | 21 |
| 1000 | 0.958 | 0.526 | 49 |
| 1004 | 0.979 | 0.941 | 19 |
| 1016 | 0.987 | 0.926 | 23 |
| 1025 | 0.894 | 0.561 | 28 |
| 1030 | 0.994 | 0.965 | 30 |
| 1033 | 0.962 | 0.865 | 26 |
| 1040 | 0.941 | 0.805 | 32 |
| 1041 | 0.981 | 0.795 | 45 |
| 1045 | 0.935 | 0.833 | 26 |
| 1046 | 0.962 | 0.558 | 46 |
| 1051 | 0.986 | 0.597 | 49 |
| 1058 | 0.975 | 0.901 | 19 |
| 1060 | 0.933 | 0.705 | 17 |
| 1062 | 0.982 | 0.513 | 49 |
| 1063 | 0.939 | 0.545 | 33 |
| 1066 | 0.945 | 0.754 | 47 |
| 1067 | 0.944 | 0.819 | 25 |
| 1072 | 0.989 | 0.673 | 44 |
| 1085 | 0.974 | 0.512 | 49 |
| 1087 | 0.958 | 0.770 | 16 |
| 1094 | 0.968 | 0.571 | 36 |
| 1095 | 0.968 | 0.617 | 42 |
| 1096 | 0.976 | 0.893 | 25 |
| 1102 | 0.961 | 0.933 | 20 |
| 1103 | 0.932 | 0.552 | 19 |
| 1114 | 0.980 | 0.568 | 47 |
| 1115 | 0.990 | 0.815 | 22 |
| 1119 | 0.985 | 0.935 | 21 |
| 1130 | 0.955 | 0.661 | 37 |
| 1134 | 0.985 | 0.614 | 21 |
| 1138 | 0.975 | 0.872 | 34 |
| 1140 | 0.965 | 0.749 | 23 |
| 1149 | 0.960 | 0.775 | 18 |
| 1151 | 0.991 | 0.727 | 34 |
| 1155 | 0.982 | 0.496 | 47 |
| 1156 | 0.944 | 0.794 | 29 |
| 1160 | 0.947 | 0.603 | 47 |
| 1162 | 0.954 | 0.745 | 31 |
| 1164 | 0.927 | 0.651 | 43 |
| 1194 | 0.948 | 0.680 | 27 |
| 1201 | 0.957 | 0.899 | 18 |
| 1207 | 0.975 | 0.935 | 18 |
| 1210 | 0.977 | 0.935 | 26 |
| 1211 | 0.985 | 0.822 | 47 |
| 1224 | 0.983 | 0.904 | 21 |
| 1228 | 0.951 | 0.544 | 47 |
| 1238 | 0.966 | 0.713 | 45 |
| 1255 | 0.939 | 0.495 | 39 |
| 1263 | 0.991 | 0.619 | 46 |
| 1266 | 0.992 | 0.806 | 39 |
| 1271 | 0.929 | 0.666 | 34 |
| 1279 | 0.966 | 0.857 | 20 |
| 1280 | 0.921 | 0.777 | 28 |
| 1282 | 0.993 | 0.927 | 19 |
| 1283 | 0.995 | 0.867 | 22 |
| 1286 | 0.965 | 0.894 | 18 |
| 1287 | 0.986 | 0.935 | 24 |
| 1288 | 0.979 | 0.733 | 32 |
| 1291 | 0.987 | 0.785 | 28 |
| 1304 | 0.920 | 0.712 | 24 |
| 1307 | 0.927 | 0.586 | 45 |
| 1308 | 0.979 | 0.963 | 18 |
| 1309 | 0.963 | 0.840 | 24 |
| 1310 | 0.967 | 0.839 | 22 |
| 1314 | 0.995 | 0.929 | 23 |
| 1316 | 0.978 | 0.873 | 24 |
| 1331 | 0.975 | 0.888 | 19 |
| 1332 | 0.961 | 0.796 | 29 |
| 1333 | 0.965 | 0.873 | 27 |
| 1339 | 0.910 | 0.631 | 25 |
| 1342 | 0.973 | 0.884 | 34 |
| 1346 | 0.933 | 0.725 | 29 |
| 1356 | 0.975 | 0.821 | 34 |
| 1365 | 0.929 | 0.632 | 39 |
| 1367 | 0.962 | 0.919 | 22 |
| 1376 | 0.955 | 0.849 | 33 |
| 1384 | 0.987 | 0.576 | 39 |
| 1389 | 0.984 | 0.889 | 26 |
| 1390 | 0.980 | 0.559 | 36 |
| 1395 | 0.974 | 0.933 | 22 |
| 1396 | 0.967 | 0.941 | 16 |
| 1398 | 0.943 | 0.829 | 24 |
| 1402 | 0.969 | 0.721 | 41 |
| 1403 | 0.934 | 0.815 | 26 |
| 1405 | 0.920 | 0.505 | 22 |
| 1409 | 0.985 | 0.939 | 24 |
| 1412 | 0.963 | 0.878 | 29 |
| 1417 | 0.957 | 0.640 | 35 |
| 1418 | 0.946 | 0.873 | 18 |
| 1419 | 0.975 | 0.645 | 37 |
| 1420 | 0.976 | 0.867 | 24 |
| 1421 | 0.985 | 0.917 | 20 |
| 1429 | 0.980 | 0.908 | 17 |
| 1430 | 0.910 | 0.712 | 28 |
| 1431 | 0.984 | 0.939 | 22 |
| 1433 | 0.974 | 0.908 | 20 |
| 1437 | 0.966 | 0.542 | 38 |
| 1438 | 0.981 | 0.941 | 27 |
| 1445 | 0.959 | 0.771 | 26 |
| 1449 | 0.988 | 0.897 | 18 |
| 1452 | 0.966 | 0.782 | 31 |
| 1455 | 0.978 | 0.944 | 22 |
| 1456 | 0.950 | 0.919 | 19 |
| 1458 | 0.974 | 0.913 | 22 |
| 1465 | 0.983 | 0.957 | 22 |
| 1467 | 0.977 | 0.937 | 20 |
| 1471 | 0.906 | 0.601 | 22 |
| 1478 | 0.959 | 0.491 | 45 |
| 1479 | 0.968 | 0.883 | 24 |
| 1480 | 0.976 | 0.916 | 22 |
| 1484 | 0.981 | 0.897 | 28 |
| 1485 | 0.972 | 0.956 | 17 |
| 1488 | 0.983 | 0.838 | 20 |
| 1489 | 0.965 | 0.869 | 18 |
| 1492 | 0.914 | 0.677 | 17 |
| 1495 | 0.945 | 0.825 | 21 |
| 1496 | 0.993 | 0.975 | 16 |
| 1498 | 0.957 | 0.891 | 19 |
| 1500 | 0.984 | 0.832 | 33 |
| 1509 | 0.996 | 0.921 | 42 |
| 1512 | 0.977 | 0.835 | 16 |
| 1515 | 0.986 | 0.945 | 20 |
| 1518 | 0.978 | 0.885 | 26 |
| 1520 | 0.996 | 0.924 | 22 |
| 1527 | 0.955 | 0.521 | 46 |
| 1528 | 0.991 | 0.502 | 48 |
| 1532 | 0.989 | 0.903 | 17 |
| 1533 | 0.987 | 0.971 | 15 |
| 1536 | 0.983 | 0.956 | 17 |
| 1538 | 0.978 | 0.915 | 25 |
| 1539 | 0.983 | 0.940 | 20 |
| 1540 | 0.965 | 0.799 | 28 |
| 1542 | 0.981 | 0.865 | 26 |
| 1545 | 0.939 | 0.502 | 9 |
| 1549 | 0.966 | 0.902 | 26 |
| 1551 | 0.990 | 0.965 | 16 |
| 1552 | 0.991 | 0.927 | 18 |
| 1554 | 0.979 | 0.895 | 31 |
| 1557 | 0.963 | 0.884 | 24 |
| 1559 | 0.977 | 0.922 | 24 |
| 1560 | 0.969 | 0.901 | 27 |
| 1561 | 0.980 | 0.946 | 25 |
| 1565 | 0.982 | 0.886 | 24 |
| 1568 | 0.962 | 0.801 | 27 |
| 1575 | 0.984 | 0.958 | 18 |
| 1576 | 0.938 | 0.818 | 25 |
| 1581 | 0.963 | 0.887 | 23 |
| 1583 | 0.970 | 0.935 | 13 |
| 1585 | 0.900 | 0.641 | 34 |
| 1587 | 0.968 | 0.902 | 19 |
| 1590 | 0.981 | 0.955 | 20 |
| 1592 | 0.966 | 0.909 | 27 |
| 1594 | 0.970 | 0.931 | 15 |
| 1597 | 0.977 | 0.905 | 23 |
| 1613 | 0.970 | 0.910 | 22 |
| 1618 | 0.976 | 0.840 | 21 |
| 1619 | 0.982 | 0.908 | 31 |
| 1622 | 0.978 | 0.879 | 26 |
| 1625 | 0.929 | 0.805 | 24 |
| 1631 | 0.991 | 0.972 | 16 |
| 1633 | 0.979 | 0.871 | 28 |
| 1635 | 0.959 | 0.816 | 19 |
| 1636 | 0.918 | 0.692 | 13 |
| 1643 | 0.990 | 0.901 | 21 |
| 1649 | 0.962 | 0.921 | 20 |
| 1654 | 0.979 | 0.865 | 31 |
| 1663 | 0.989 | 0.966 | 16 |
| 1667 | 0.980 | 0.916 | 37 |
| 1669 | 0.953 | 0.496 | 34 |
| 1670 | 0.988 | 0.951 | 16 |
| 1672 | 0.966 | 0.913 | 17 |
| 1681 | 0.927 | 0.671 | 20 |
| 1685 | 0.967 | 0.593 | 36 |
| 1695 | 0.986 | 0.919 | 18 |
| 1697 | 0.898 | 0.716 | 24 |
| 1702 | 0.940 | 0.837 | 19 |
| 1703 | 0.994 | 0.620 | 43 |

### Example 7

### Prediction of Transmembrane Regions of the Polypeptides of the Invention

Regions of the polypeptides of the invention that cross a cellular lipid bilayer or membrane were predicted using the TMPRED computer program (Hofmann et al. Biol. Chem. Hoppe-Seyler 374,166 hereby incorporated herein by reference). The following table describes the plurality of polypeptides of the invention that contain at least one predicted transmembrane domain:

TABLE 6

| | Number of | | |
|---|---|---|---|
| | Predicted | | Domain |
| | Transmembrane | Total | Location: |
| SEQ ID NO: | Regions | Score | Score |
| 854 | 1 | 2088 | 115-138: 2088 |
| 864 | 6 | 12660 | 56-78: 2631 |
| | | | 150-169: 1686 |
| | | | 243-258: 1737 |
| | | | 336-359: 1556 |
| | | | 379-394: 2572 |
| | | | 456-473: 2478 |
| 871 | 1 | 1947 | 434-454: 1947 |
| 873 | 4 | 8479 | 53-69: 2714 |
| | | | 136-152: 2014 |
| | | | 203-220: 1906 |
| | | | 509-526: 1845 |
| 875 | 1 | 1722 | 118-138: 1722 |
| 878 | 4 | 7527 | 68-86: 1551 |
| | | | 105-123: 1875 |
| | | | 272-290: 1556 |
| | | | 340-356: 2545 |
| 879 | 6 | 11423 | 127-148: 1678 |
| | | | 281-300: 2640 |
| | | | 393-412: 1662 |
| | | | 427-447: 1670 |
| | | | 543-559: 2249 |
| | | | 638-653: 1524 |
| 886 | 4 | 8588 | 104-120: 2019 |
| | | | 243-267: 2346 |
| | | | 335-350: 2222 |
| | | | 401-425: 2001 |
| 894 | 3 | 7997 | 141-156: 3090 |
| | | | 167-190: 1601 |
| | | | 207-226: 3306 |
| 896 | 1 | 2642 | 44-66: 2642 |
| 898 | 1 | 2578 | 87-106: 2578 |
| 908 | 1 | 1569 | 958-974: 1569 |
| 914 | 1 | 3089 | 502-524: 3089 |
| 918 | 1 | 2577 | 469-485: 2577 |
| 919 | 1 | 2819 | 233-254: 2819 |
| 926 | 3 | 6476 | 161-177: 2207 |
| | | | 254-270: 2180 |
| | | | 449-467: 2089 |
| 929 | 1 | 2228 | 121-136: 2228 |
| 934 | 6 | 12682 | 172-187: 2293 |
| | | | 237-258: 2056 |
| | | | 279-299: 1759 |
| | | | 329-351: 2812 |
| | | | 371-386: 2117 |
| | | | 408-429: 1645 |
| 937 | 1 | 2548 | 248-264: 2548 |
| 939 | 1 | 2181 | 93-114: 2181 |
| 941 | 2 | 3480 | 89-110: 1685 |
| | | | 135-154: 1795 |
| 946 | 1 | 2450 | 385-401: 2450 |
| 957 | 1 | 2151 | 97-117: 2151 |
| 960 | 3 | 5457 | 598-614: 1857 |
| | | | 707-724: 1544 |
| | | | 748-764: 2056 |
| 968 | 2 | 4661 | 49-71: 2672 |
| | | | 165-185: 1989 |
| 969 | 4 | 7843 | 89-105: 2039 |
| | | | 164-183: 2176 |
| | | | 224-243: 1514 |
| | | | 282-299: 2114 |
| 971 | 1 | 2123 | 100-118: 2123 |
| 978 | 1 | 2143 | 135-148: 2143 |
| 979 | 1 | 2131 | 49-66: 2131 |
| 986 | 1 | 1710 | 128-143: 1710 |
| 989 | 1 | 1979 | 112-130: 1979 |
| 997 | 1 | 1929 | 288-303: 1929 |
| 999 | 1 | 3084 | 178-199: 3084 |
| 1005 | 1 | 3072 | 73-90: 3072 |
| 1008 | 1 | 3073 | 641-660: 3073 |
| 1012 | 1 | 2019 | 910-925: 2019 |
| 1015 | 1 | 3454 | 396-416: 3454 |
| 1016 | 1 | 3369 | 581-605: 3369 |
| 1017 | 1 | 1618 | 644-663: 1618 |
| 1027 | 6 | 13013 | 80-96: 2887 |
| | | | 150-169: 2073 |
| | | | 227-243: 2086 |
| | | | 307-324: 1960 |
| | | | 404-419: 1773 |
| | | | 481-495: 2234 |
| 1028 | 1 | 1790 | 92-107: 1790 |
| 1030 | 1 | 1710 | 63-86: 1710 |
| 1035 | 1 | 2506 | 94-111: 2506 |
| 1040 | 1 | 1561 | 718-733: 1561 |
| 1041 | 4 | 8350 | 71-88: 1896 |
| | | | 291-307: 2812 |
| | | | 387-406: 1755 |
| | | | 474-490: 1887 |
| 1062 | 1 | 3368 | 258-278: 3368 |
| 1065 | 1 | 2643 | 336-358: 2643 |
| 1066 | 1 | 2406 | 49-70: 2406 |
| 1067 | 1 | 2700 | 493-510: 2700 |
| 1073 | 2 | 4203 | 135-168: 2597 |
| | | | 200-214: 1606 |
| 1075 | 4 | 6874 | 50-66: 1560 |
| | | | 139-155: 1995 |
| | | | 190-213: 1787 |
| | | | 291-305: 1532 |
| 1080 | 3 | 7420 | 116-132: 2923 |
| | | | 197-212: 2709 |
| | | | 223-239: 1788 |
| 1085 | 1 | 2097 | 195-216: 2097 |
| 1088 | 1 | 1996 | 74-89: 1996 |
| 1090 | 7 | 13484 | 68-88: 2007 |
| | | | 105-119: 1501 |
| | | | 141-159: 1687 |
| | | | 220-238: 2030 |
| | | | 296-311: 2033 |
| | | | 395-410: 1727 |
| | | | 452-471: 2499 |
| 1101 | 1 | 2000 | 403-423: 2000 |
| 1102 | 1 | 3504 | 743-764: 3504 |
| 1105 | 1 | 2219 | 351-369: 2219 |
| 1106 | 6 | 12120 | 68-88: 2342 |
| | | | 128-145: 1914 |
| | | | 185-206: 1521 |
| | | | 312-329: 2024 |
| | | | 365-380: 2531 |
| | | | 433-448: 1788 |
| 1111 | 1 | 1517 | 51-68: 1517 |
| 1113 | 1 | 3180 | 356-380: 3180 |
| 1116 | 1 | 2206 | 56-75: 2206 |
| 1124 | 2 | 4091 | 83-111: 2452 |
| | | | 130-148: 1639 |
| 1125 | 1 | 2998 | 160-181: 2998 |
| 1129 | 1 | 3163 | 779-799: 3163 |
| 1130 | 1 | 2744 | 102-126: 2744 |
| 1133 | 1 | 2029 | 112-129: 2029 |
| 1134 | 1 | 2206 | 736-757: 2206 |
| 1140 | 1 | 2750 | 87-99: 2750 |
| 1142 | 1 | 2922 | 105-123: 2922 |
| 1148 | 1 | 3113 | 294-316: 3113 |
| 1164 | 5 | 9520 | 483-502: 1826 |
| | | | 516-531: 1837 |
| | | | 584-605: 2275 |
| | | | 626-642: 1515 |
| | | | 674-690: 2067 |
| 1165 | 6 | 11860 | 92-112: 2331 |
| | | | 121-141: 1535 |
| | | | 209-230: 2031 |
| | | | 336-353: 1755 |
| | | | 389-406: 2107 |
| | | | 457-472: 2101 |
| 1166 | 4 | 7475 | 164-179: 1986 |
| | | | 229-248: 2015 |
| | | | 254-269: 1577 |
| | | | 286-306: 1897 |
| 1168 | 6 | 12530 | 109-122: 2345 |
| | | | 195-212: 1649 |
| | | | 344-366: 2101 |
| | | | 424-439: 2493 |
| | | | 455-474: 2000 |
| | | | 476-496: 1942 |
| 1170 | 1 | 2318 | 349-376: 2318 |
| 1175 | 1 | 1582 | 90-108: 1582 |
| 1176 | 1 | 1516 | 742-764: 1516 |
| 1179 | 1 | 2508 | 135-154: 2508 |
| 1185 | 1 | 1649 | 58-74: 1649 |
| 1190 | 1 | 2235 | 296-312: 2235 |
| 1194 | 1 | 2823 | 44-70: 2823 |
| 1196 | 1 | 2041 | 187-202: 2041 |
| 1199 | 1 | 3788 | 99-116: 3788 |
| 1201 | 1 | 1553 | 551-566: 1553 |
| 1207 | 4 | 8078 | 134-150: 2201 |
| | | | 237-258: 1652 |
| | | | 375-397: 2178 |
| | | | 484-501: 2047 |
| 1210 | 1 | 2222 | 118-132: 2222 |
| 1233 | 1 | 1704 | 49-67: 1704 |
| 1255 | 1 | 2677 | 84-106: 2677 |
| 1266 | 3 | 7330 | 59-76: 2685 |
| | | | 90-108: 1580 |
| | | | 195-221: 3065 |
| 1271 | 2 | 3771 | 634-650: 1766 |
| | | | 691-709: 2005 |
| 1273 | 2 | 4814 | 85-105: 2193 |
| | | | 216-241: 2621 |
| 1280 | 3 | 6011 | 58-74: 2642 |
| | | | 171-188: 1509 |
| | | | 228-250: 1860 |
| 1301 | 1 | 1651 | 112-131: 1651 |
| 1304 | 1 | 1960 | 187-209: 1960 |
| 1308 | 1 | 2729 | 212-229: 2729 |
| 1309 | 1 | 2154 | 564-579: 2154 |
| 1316 | 1 | 1634 | 90-105: 1634 |
| 1320 | 1 | 1847 | 134-150: 1847 |
| 1322 | 2 | 5403 | 83-98: 2412 |
| | | | 160-175: 2991 |
| 1324 | 1 | 2129 | 128-147: 2129 |
| 1327 | 1 | 1645 | 115-131: 1645 |
| 1333 | 1 | 2378 | 122-138: 2378 |
| 1334 | 1 | 2681 | 52-76: 2681 |
| 1342 | 1 | 1520 | 365-380: 1520 |
| 1343 | 1 | 2312 | 139-155: 2312 |
| 1356 | 1 | 2292 | 101-129: 2292 |
| 1376 | 1 | 1606 | 606-621: 1606 |
| 1377 | 4 | 8637 | 100-113: 2419 |
| | | | 294-313: 2702 |
| | | | 787-808: 1889 |
| | | | 985-1001: 1627 |
| 1379 | 2 | 5222 | 197-218: 2416 |
| | | | 277-297: 2806 |
| 1380 | 1 | 2398 | 102-120: 2398 |
| 1389 | 4 | 9882 | 177-192: 3257 |
| | | | 235-261: 2052 |
| | | | 276-291: 1761 |
| | | | 363-381: 2812 |
| 1393 | 2 | 4456 | 66-82: 2701 |
| | | | 110-126: 1755 |
| 1395 | 2 | 3335 | 263-279: 1511 |
| | | | 325-342: 1824 |
| 1400 | 1 | 1793 | 95-113: 1793 |
| 1402 | 2 | 4648 | 229-245: 1641 |
| | | | 313-333: 3007 |
| 1417 | 2 | 3802 | 92-114: 2061 |
| | | | 152-175: 1741 |
| 1425 | 6 | 13420 | 59-75: 1823 |
| | | | 112-127: 3392 |
| | | | 132-147: 1707 |
| | | | 180-193: 2193 |
| | | | 231-246: 1810 |
| | | | 331-346: 2495 |
| 1428 | 1 | 1923 | 60-78: 1923 |
| 1434 | 1 | 3041 | 111-129: 3041 |
| 1436 | 2 | 4541 | 103-118: 2057 |
| | | | 295-309: 2484 |
| 1444 | 1 | 1540 | 257-275: 1540 |
| 1445 | 1 | 2610 | 199-219: 2610 |
| 1449 | 3 | 6892 | 143-158: 1938 |
| | | | 181-198: 2860 |
| | | | 270-289: 2094 |
| 1455 | 1 | 2481 | 241-256: 2481 |
| 1459 | 5 | 10350 | 115-133: 2128 |
| | | | 183-195: 2109 |
| | | | 229-245: 1520 |
| | | | 288-307: 2678 |
| | | | 356-372: 1915 |
| 1478 | 1 | 2281 | 56-71: 2281 |
| 1484 | 1 | 2689 | 229-245: 2689 |
| 1495 | 1 | 2478 | 146-162: 2478 |
| 1496 | 2 | 5761 | 68-84: 2749 |
| | | | 144-163: 3012 |
| 1500 | 3 | 6144 | 81-99: 2768 |
| | | | 118-132: 1551 |
| | | | 166-182: 1825 |
| 1511 | 1 | 1528 | 76-91: 1528 |
| 1515 | 1 | 1775 | 201-233: 1775 |
| 1516 | 1 | 3335 | 71-93: 3335 |
| 1523 | 3 | 5593 | 128-145: 1794 |
| | | | 348-365: 1730 |
| | | | 438-455: 2069 |
| 1556 | 1 | 1521 | 147-166: 1521 |
| 1559 | 1 | 2828 | 628-650: 2828 |
| 1565 | 1 | 2718 | 306-332: 2718 |
| 1567 | 1 | 2660 | 54-72: 2660 |
| 1574 | 1 | 1922 | 504-519: 1922 |
| 1575 | 1 | 2773 | 306-332: 2773 |
| 1576 | 1 | 2661 | 218-239: 2661 |
| 1583 | 1 | 2635 | 352-369: 2635 |
| 1587 | 1 | 3196 | 518-538: 3196 |
| 1589 | 1 | 2059 | 78-99: 2059 |
| 1593 | 4 | 8614 | 164-178: 2254 |
| | | | 259-274: 2312 |
| | | | 325-340: 2378 |
| | | | 392-410: 1670 |
| 1594 | 1 | 2673 | 339-357: 2673 |
| 1616 | 1 | 2581 | 120-136: 2581 |
| 1618 | 1 | 3544 | 324-344: 3544 |
| 1619 | 1 | 1551 | 695-711: 1551 |
| 1622 | 1 | 1516 | 765-780: 1516 |
| 1635 | 1 | 3694 | 1258-1274: 3694 |
| 1638 | 1 | 1606 | 105-121: 1606 |
| 1640 | 1 | 4013 | 2045-2062: 4013 |
| 1645 | 1 | 1722 | 80-96: 1722 |
| 1655 | 1 | 2392 | 68-90: 2392 |
| 1673 | 8 | 14475 | 61-80: 1745 |
| | | | 129-154: 1690 |
| | | | 235-251: 1560 |
| | | | 262-277: 1971 |
| | | | 350-368: 2264 |
| | | | 398-415: 1642 |
| | | | 521-537: 1609 |
| | | | 559-577: 1994 |
| 1680 | 1 | 2286 | 145-162: 2286 |
| 1684 | 3 | 6111 | 131-149: 2670 |
| | | | 162-176: 1558 |
| | | | 223-243: 1883 |
| 1685 | 1 | 3127 | 709-725: 3127 |
| 1686 | 4 | 8443 | 73-88: 2001 |
| | | | 114-132: 2487 |
| | | | 143-168: 1531 |
| | | | 288-311: 2424 |
| 1687 | 1 | 2972 | 285-303: 2972 |
| 1701 | 1 | 3206 | 88-113: 3206 |

### Example 8

### Prediction of Chromosomal Location of the Polynucleotides of the Invention

Chromosomal location of the polynucleotide sequences of the invention was determined by using the BLASTN algorithm (Altschul et al. Nucleic Acids Res. 25 pp. 3389-3402 herein incorporated by reference). SEQ ID NO: 1 through 852 were aligned to the sequence of the human genome (Version date Nov. 3, 2002 from Genbank) using the BLASTN computer program. The chromosomal locations of the aligned human genome sequences were determined through the Genbank accession numbers of the aligned human genome sequences, which enabled retrieval of annotated chromosomal location from the Genbank database. The following table depicts the predicted chromosomal location of the polynucleotides of the invention (SEQ ID NO 1 through 852).

TABLE 7

| SEQ ID NO: | Chromsomal_location |
|---|---|
| 1 | 11q |
| 2 | 11q |
| 3 | 10 |
| 4 | 3 |
| 6 | 16 |
| 7 | 15 |
| 8 | 6 |
| 10 | 5 |
| 12 | 1 |
| 13 | 15 |
| 14 | 7 |
| 18 | 5 |
| 19 | 1 |
| 20 | 14 |
| 21 | 11 |
| 22 | Xp11.3-11.4 |
| 23 | 11 |
| 25 | 19 |
| 26 | 1 |
| 28 | 11 |
| 29 | 19 |
| 32 | 9 |
| 34 | 17 |
| 35 | 19 |
| 36 | 3 |
| 37 | 17 |
| 38 | 4 |
| 40 | 4 |
| 41 | 6 |
| 43 | 2 |
| 44 | 19 |
| 45 | 11 |
| 46 | X |
| 47 | 1 |
| 49 | 20 |
| 50 | 1 |
| 51 | 5 |
| 52 | 1 |
| 53 | 19 |
| 55 | 19 |
| 56 | 4 |
| 57 | 4 |
| 59 | 11q |
| 60 | 17 |
| 62 | 10 |
| 63 | 5 |
| 64 | 15 |
| 65 | 16 |
| 67 | 20 |
| 68 | Xq22.1-23 |
| 70 | 10 |
| 71 | 8 |
| 72 | 21 |
| 73 | 2 |
| 75 | 8 |
| 76 | 3 |
| 77 | 9 |
| 78 | 7 |
| 79 | 19 |
| 80 | 16 |
| 81 | 3 |
| 82 | 5 |
| 83 | 13 |
| 84 | 1p36.11-36.31 |
| 85 | 1p32.1-33 |
| 86 | 1p36.11-36.31 |
| 87 | 8 |
| 88 | 3 |
| 89 | 15 |
| 90 | 6p24.1-25.3 |
| 91 | 1 |
| 94 | 3 |
| 95 | 20 |
| 96 | 17 |
| 97 | 4 |
| 99 | 19 |
| 101 | 2 |
| 103 | 11q |
| 104 | 2 |
| 105 | X |
| 106 | 19 |
| 108 | 4 |
| 109 | 13q12.11-12.3 |
| 110 | 17 |
| 111 | 12 |
| 112 | 2 |
| 113 | X |
| 114 | 3 |
| 117 | 10 |
| 118 | 5 |
| 119 | 16 |
| 120 | 19 |
| 121 | X |
| 122 | 1 |
| 123 | 5 |
| 124 | 12 |
| 125 | 1 |
| 126 | 16 |
| 127 | 11q |
| 128 | 1 |
| 129 | 4 |
| 130 | 9q13-21.2 |
| 131 | 10 |
| 132 | 19 |
| 133 | 10 |
| 134 | 13 |
| 135 | 17 |
| 136 | 11q |
| 137 | 8 |
| 138 | 2 |
| 140 | 5 |
| 141 | 6 |
| 142 | 1 |
| 143 | 19 |
| 144 | 5 |
| 146 | 3 |
| 147 | 5 |
| 148 | 11 |
| 149 | 7 |
| 151 | 19 |
| 152 | 14 |
| 154 | 16 |
| 155 | 3 |
| 156 | 16 |
| 157 | 9p23-24.3 |
| 160 | 20 |
| 162 | 10 |
| 163 | 1 |
| 164 | 20p11.1-11.23 |
| 165 | 3 |
| 166 | 5 |
| 167 | 1 |
| 168 | 6 |
| 169 | 14 |
| 170 | 10 |
| 171 | 8 |
| 172 | 11 |
| 173 | 7 |
| 174 | 3 |
| 175 | 2 |
| 176 | 1p36.21-36.33 |
| 177 | 19 |
| 180 | 1q25.1-25.3 |
| 181 | X |
| 183 | 4 |
| 184 | 7 |
| 185 | 10 |
| 186 | 19 |
| 187 | 11q |
| 188 | 10 |
| 189 | Xp |
| 191 | 16 |
| 192 | 8 |
| 193 | 2 |
| 195 | 3p |
| 196 | 1 |
| 197 | 13 |
| 198 | 6q26-27 |
| 199 | 13 |
| 201 | 11 |
| 202 | 4 |
| 203 | 4 |
| 204 | 11 |
| 205 | 5 |
| 206 | 1 |
| 207 | 16 |
| 208 | 3 |
| 209 | 20 |
| 210 | 3 |
| 212 | 11 |
| 213 | 1 |
| 214 | 20 |
| 215 | 8 |
| 216 | 11q |
| 217 | 11q |
| 218 | 13 |
| 219 | 3 |
| 222 | 19 |
| 223 | 16 |
| 224 | 20 |
| 226 | 3 |
| 227 | 9q22.1-22.33 |
| 228 | 12 |
| 229 | 6p12.3-21.2 |
| 230 | 11q |
| 233 | 7 |
| 235 | 1 |
| 236 | 7 |
| 237 | 12 |
| 238 | 14 |
| 239 | 10 |
| 240 | 19 |
| 241 | 22q11.2 |
| 242 | 8 |
| 243 | 1q24 |
| 244 | 17 |
| 245 | 4 |
| 246 | 7 |
| 247 | 17 |
| 249 | 6 |
| 250 | 18 |
| 251 | 1 |
| 252 | 1 |
| 253 | 1 |
| 255 | Xq23 |
| 256 | 12 |
| 258 | 1 |
| 259 | 22 |
| 260 | X |
| 261 | 1p31.2-32.3 |
| 263 | 6 |
| 264 | 3 |
| 265 | 17 |
| 266 | 16 |
| 268 | 10 |
| 270 | 2 |
| 271 | 2 |
| 272 | 9 |
| 273 | 5 |
| 274 | 1 |
| 275 | 9 |
| 276 | 1 |
| 277 | 18 |
| 278 | 21 |
| 281 | 12 |
| 282 | 1 |
| 284 | 5 |
| 285 | 3 |
| 286 | 5 |
| 287 | 12 |
| 289 | 6 |
| 290 | 3 |
| 291 | 10 |
| 292 | 16 |
| 293 | 3 |
| 294 | 1p31.2-32.3 |
| 295 | 4 |
| 296 | 11q |
| 297 | 9 |
| 298 | 10 |
| 299 | 19 |
| 300 | 7 |
| 302 | 2 |
| 303 | 1 |
| 304 | 17 |
| 305 | 14 |
| 306 | 6q22.22-23.3 |
| 307 | 11 |
| 308 | 16 |
| 310 | 5 |
| 312 | 19 |
| 313 | 1 |
| 314 | 6 |
| 315 | 19 |
| 316 | 16 |
| 317 | 12 |
| 320 | 6 |
| 321 | 12 |
| 322 | 1 |
| 324 | 15 |
| 325 | 10 |
| 326 | 4 |
| 327 | 11 |
| 328 | 1 |
| 329 | 1q24.1-25.2 |
| 330 | 9 |
| 332 | 6q14.2-16.1 |
| 333 | 19 |
| 334 | 7 |
| 335 | 19 |
| 336 | 5 |
| 338 | 22 |
| 339 | 22 |
| 340 | 1 |
| 341 | 3 |
| 342 | 16 |
| 343 | 9 |
| 344 | 19 |
| 345 | 5 |
| 346 | 20p11.21-11.23 |
| 347 | Xp11.4-21.2 |
| 348 | 8 |
| 349 | 7 |
| 351 | 17 |
| 352 | 6 |
| 354 | 1 |
| 355 | 2 |
| 359 | 11 |
| 361 | 9 |
| 368 | 11 |
| 370 | 20 |
| 374 | 17 |
| 381 | 9 |
| 387 | 5 |
| 388 | 8 |
| 389 | 9 |
| 391 | 16 |
| 393 | 1 |
| 395 | 5 |
| 396 | 19 |
| 399 | 2 |
| 400 | 1 |
| 407 | 5 |
| 410 | 3 |
| 413 | 6 |
| 415 | 1 |
| 417 | 5 |
| 419 | 16 |
| 420 | 10 |
| 421 | 6 |
| 424 | X |
| 429 | 1 |
| 431 | 9 |
| 433 | 14 |
| 434 | 1 |
| 435 | 1 |
| 436 | 3 |
| 437 | 5 |
| 438 | 7 |
| 441 | 11 |
| 445 | 14 |
| 446 | 10 |
| 448 | 1 |
| 449 | 19 |
| 450 | X |
| 451 | X |
| 455 | 10 |
| 457 | Xq25-Xq26 |
| 460 | 14 |
| 461 | 17 |
| 463 | 15 |
| 466 | 3 |
| 468 | 18 |
| 471 | 10 |
| 473 | 11 |
| 474 | 1 |
| 477 | 5 |
| 478 | 3 |
| 480 | 19 |
| 488 | 10 |
| 495 | 5 |
| 498 | 19 |
| 500 | 1 |
| 505 | 9 |
| 506 | 2 |
| 507 | 1 |
| 509 | 8 |
| 516 | X |
| 520 | 1 |
| 522 | 1p36.1-36.2 |
| 526 | 1p36.13-36.31 |
| 531 | 5 |
| 532 | 7 |
| 533 | 5 |
| 535 | 5 |
| 540 | 9 |
| 545 | 16 |
| 548 | 12 |
| 549 | 8 |
| 557 | 7 |
| 559 | 4 |
| 561 | 2 |
| 562 | 1p35.1-36.13 |
| 563 | 3 |
| 564 | 3 |
| 566 | 12 |
| 570 | 10 |
| 571 | 3 |
| 572 | 3 |
| 573 | 15 |
| 574 | 13 |
| 582 | 4 |
| 585 | 11 |
| 586 | 1 |
| 591 | 3 |
| 592 | 14 |
| 595 | 1 |
| 596 | 19 |
| 597 | 3 |
| 598 | 11 |
| 601 | 12 |
| 604 | 1 |
| 606 | 11 |
| 607 | 17 |
| 609 | 11 |
| 611 | 17 |
| 613 | 10 |
| 618 | 3 |
| 619 | 16 |
| 624 | 8 |
| 627 | 3p21.3 |
| 629 | 10 |
| 631 | 6 |
| 637 | 3 |
| 638 | 6q16-21 |
| 639 | 11q |
| 641 | 2 |
| 646 | 11 |
| 648 | 7 |
| 651 | 4 |
| 654 | 4 |
| 655 | 9 |
| 656 | 14 |
| 660 | X |
| 663 | 6 |
| 667 | 6 |
| 669 | 12 |
| 670 | 20p12.1-13 |
| 671 | 11 |
| 672 | 1 |
| 676 | 11 |
| 682 | 20 |
| 687 | 19 |
| 689 | 17 |
| 693 | 2 |
| 696 | 9 |
| 699 | 11 |
| 703 | 15 |
| 704 | 14 |
| 705 | 17 |
| 707 | 1 |
| 710 | 1 |
| 714 | 1 |
| 715 | 21q21.1-q21.2 |
| 717 | 5 |
| 718 | 22 |
| 720 | 9 |
| 721 | 11 |
| 725 | 12q |
| 726 | 16 |
| 727 | 16 |
| 728 | 11 |
| 733 | 19 |
| 737 | 1 |
| 739 | 19 |
| 742 | 19 |
| 746 | 14 |
| 748 | 3 |
| 749 | 1 |
| 752 | 20 |
| 754 | 6 |
| 755 | 18 |
| 756 | 7 |
| 760 | 19 |
| 765 | Xq22.1-22.3 |
| 766 | 17 |
| 768 | 1 |
| 770 | 3 |
| 771 | 11 |
| 772 | 10 |
| 775 | 19 |
| 776 | 19 |
| 777 | 14 |
| 778 | 3 |
| 782 | 17 |
| 783 | 1 |
| 787 | Xq12-13.1 |
| 789 | 17 |
| 791 | 1 |
| 793 | 1 |
| 794 | 3 |
| 799 | 15 |
| 800 | X |
| 804 | 19 |
| 808 | 11 |
| 809 | 3 |
| 812 | 19 |
| 813 | X |
| 814 | 6 |
| 818 | 4 |
| 824 | 11q |
| 826 | 15 |
| 829 | 17 |
| 830 | 5 |
| 831 | 11 |
| 833 | 8 |
| 834 | 11 |
| 835 | 22q12.2-13.1 |
| 836 | 18 |
| 839 | 21q22.2 |

### Example 9

### Purification of the Polypeptides of the Invention

The polypeptides of the invention can be produced by first cloning the corresponding polynucleotides of the invention into a mammalian expression vector using established protocols that are familiar to those in skill of the art (for example, pCDNA3.1/His A, B, C Catalog Number V385-20 from Invitrogen, Carlsbad, Calif., herein incorporated by reference). Briefly, polynucleotides of the invention are amplified and isolated via agarose gel electrophoresis and gel excision. The polynucleotides are subsequently cloned into the pCDNA3.1/His A, B, C vectors to facilitate in-frame cloning with a polyhistidine metal-binding tag. The vectors with polynucleotides of the invention inserted can then be transfected into a mammalian cell line like COS-7 for high-level expression of the corresponding polypeptides of the invention.

The corresponding polypeptides of the invention that are produced by the transfected mammalian cell line can then be purified according to established protocols that are familiar to those of skill in the art (for example, the Ni-NTA Magnetic Agarose Beads protein purification protocol, Qiagen, Valencia, Calif., herein incorporated by reference). Briefly, assays utilizing Ni-NTA Magnetic Beads involve capture of the 6×His-tagged protein—from a cell lysate or a purified-protein solution—followed by washing, binding of interaction partners, further washing, and finally elution of the interacting partner from the still immobilized 6×His-tagged protein or elution of the interacting-partner-6×His-tagged-protein complex. Between each step, the beads are collected by attracting them to the side of the vessel, after placing near a magnet for 30-60 seconds. Purification procedures may even use crude cell extracts for binding of 6×His-tagged protein.

The resulting purified polypeptides of the invention are then quantified, lyophilized and stored at 4 degrees Celcius or below, and used for standards in diagnostic kits for pre-eclampsia.

### Example 10

### Production of Specific Antibodies for the Polypeptides of the Invention

Cells expressing any of the polypeptides of the invention are identified using antibodies specific for SEQ ID NO: 853 through 1704. Polyclonal antibodies are produced by DNA vaccination or by injection of peptide antigens into rabbits or other hosts. An animal, such as a rabbit, is immunized with a peptide specific for one of SEQ ID NO: 853 through 1704 conjugated to a carrier protein, such as BSA (bovine serum albumin) or KLH (keyhole limpet hemocyanin). The rabbit is initially immunized with conjugated peptide in complete Freund's adjuvant, followed by a booster shot every two weeks with injections of conjugated peptide in incomplete Freund's adjuvant. Antibodies specific for a polypeptide of the invention are affinity purified from rabbit serum using peptides corresponding to the polypeptides of the invention coupled to Affi-Gel 10 (Bio-Rad), and stored in phosphate-buffered saline with 0.1% sodium azide. To determine that the polyclonal antibodies are specific for a polypeptide of the invention, an expression vector encoding a polypeptide of the invention is introduced into mammalian cells. Western blot analysis of protein extracts of non-transfected cells and the cells containing a polypeptide of the invention is performed using the polyclonal antibody sample as the primary antibody and a horseradish peroxidase-labeled anti-rabbit antibody as the secondary antibody. Detection of a band in the cells containing a polypeptide of the invention and lack thereof in the control cells indicates that the polyclonal antibodies are specific for the polypeptide of the invention in question.

Monoclonal antibodies are produced by injecting mice with a peptide derived from a polypeptide of the invention, with or without adjuvant. Subsequently, the mouse is boosted every 2 weeks until an appropriate immune response has been identified (typically 1-6 months), at which point the spleen is removed. The spleen is minced to release splenocytes, which are fused (in the presence of polyethylene glycol) with murine myeloma cells. The resulting cells (hybridomas) are grown in culture and selected for antibody production by clonal selection. The antibodies are secreted into the culture supernatant, facilitating the screening process, such as screening by an enzyme-linked immunosorbent assay (ELISA). Alternatively, humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above.

### Example 11

### Determination of mRNA Expression of the Polynucleotides of the Invention in Normal and Pre-Eclampic Placental Tissues

Relative gene expression, at the mRNA level, across multiple placental tissues was measured using a real-time PCR system. This system consisted of a GeneAmp 5700 Sequence Detection System (Applied Biosystems) and GeneAmp 5700 SDS software (Applied Biosystems). The reaction mixture consists of SYBR Green PCR MasterMix (Applied Biosystems), oligos specific to one gene, and DEPC treated water. 20 ng mRNA equivalent cDNA (synthesized using MMLV RT enzyme from Invitrogen) is added to each reaction mixture. The samples are run on the GeneAmp 5700 Sequencing Detection System with a 30 second extension time and are analyzed at a fluorescence threshold of 0.5. Each cDNA is also run against an appropriate Housekeeping Gene Control that is used for normalization.

The real-time PCR protocol was performed on both SEQ ID NO: 357 and 551. After this analysis was performed, the relative expression ratios of the two polynucleotides of the invention were determined across a selection of placental tissues. The raw cycle count scores for each gene in the measured tissues were selected, with each score representing the number of doublings required to detect the message. Next, the inverse expression ratios were computed for each gene in each measured tissue using the following formula:
(2 raised to the power of the SEQ ID NO 347 raw cycle count score) divided by (2 raised to the power of the SEQ ID NO 551 raw cycle count score)

Finally, the computed ratios for each measured tissue were plotted on a log 10 scale as shown in FIG. 1 of the drawings. The following table relates the X-axis labels of FIG. 1 to the respective placental tissue source information.

TABLE 8

| Label on X-axis | |
|---|---|
| for FIG. 1 | Detailed Tissue Source Information |
| 10.1. PE BP | 23 weeks of pregnancy, severe pre-eclampsia, basal- |
| | plate region |
| 1.1 PE BP | 23 weeks of pregnancy, severe pre-eclampsia, basal- |
| | plate region |
| 7.1 PE BP | 24 weeks of pregnancy, severe pre-eclampsia, basal- |
| | plate region |
| 4.1 PE BP | 29 weeks of pregnancy, severe pre-eclampsia, basal- |
| | plate region |
| 5.1 PE BP | 32 weeks of pregnancy, severe pre-eclampsia, basal- |
| | plate region |
| 11.1 PE BP | 38 weeks of pregnancy, pre-eclampsia, basal-plate |
| | region |
| ECL003 | 34 weeks of pregnancy, non-pre-eclamptic placenta, |
| NP BP | basal-plate region |
| 9.1 NP BP | 39 weeks of pregnancy, non-pre-eclamptic placenta, |
| | basal-plate region |
| 8.1 NP BP | 40 weeks of pregnancy, non-pre-eclamptic placenta, |
| | basal-plate region |
| 6.3 NP BP | 41 weeks of pregnancy, non-pre-eclamptic placenta, |
| | basal-plate region |
| 13.1 NP BP | 39 weeks of pregnancy, elective Caesarian section, |
| | non-pre-eclamptic placenta, basal-plate region |

### Example 12

### Diagnostic Kit for Polypeptides of the Invention

A diagnostic kit can be made in order to determine the levels of selected polypeptides of the invention that are present in tissue samples obtained from a patient who is undergoing pregnancy. Such tissue samples may be from blood plasma, blood serum, amniotic fluid, or urine.

The components of the kit can be as follows: 1) one or more standards comprised of one or more polypeptides of the invention, prepared as described in example 9; 2) an antibody or a plurality of antibodies that are specific for the polypeptides of the invention that are being tested by the kit, prepared as described in example 10; 3) written instructions; 4) diluents for tissue samples and the standards; 5) a wash buffer; 6) color reagents; 7) stop solution; and 8) a microplate with bound antibody.

An example of such a kit is an ELISA (enzyme-linked immunosorbent assay) quantification that determines the concentration or concentrations of a polypeptide or polypeptides of the invention. The principle of the assay is to use the quantitative sandwich enzyme immunoassay technique where a Polyclonal antibody selective for binding a polypeptide of the invention is pre-coated onto a microplate into its wells. The standards and sample are then pipetted into the wells and any of the polypeptide of the invention that is present is bound to this immobilized Polyclonal antibody. Next, the wells are washed with washing buffer, and an enzyme-linked Polyclonal antibody that is specific for the polypeptide of the invention is added to the wells. Washing again is performed, then a substrate solution is added to the wells. Color subsequently develops in proportion to the amount of polypeptide of the invention that is bound in the first step. The color development is stopped using a stop solution, and the intensity of the color is measured by a microplate reader.

This results of this assay are used to determine one or more concentration levels of the polypeptides of the invention. The assay reaction is performed in the same tube or well, or may be in separte tubes or wells. The level or levels of polypeptides of the invention that are being determined by this assay can be used to diagnose those patients with pre-eclampsia.

### Example 13

### Diagnostic Chip for Polypeptides of the Invention in Tissue Samples

The levels of polypeptides of the invention in biological tissue samples can be determined through the use of ProteinChip technology (Wright Jr., et al. Expert Review of Molecular Diagnositcs 2002, Vol. 2 pp. 549-63 hereby incorporated herein by reference). This process includes the additon of a few microliters of tissue sample onto the ProteinChip surface, which has both chemical (e.g., anionic, catioinic, hydrophobic, hydrophilic, metal) and biochemical (antibody, receptor, DNA, enzyme probes attached to its surface. Subsequently, binding occurs between the polypeptides of the invention and their respective probes, and the chip is washed at least three times in order to elute unbound protein, lipids, salts, and other substances. Next, an energy-absorbent molecule (EAM) such as sinapinic acid is added to the chip, and the chip is placed into a ProteinChip Reader which measures the molecular mass of the bound polypeptides. Peaks are subsequently produced from the ProteinChip Reader that are used to quantify the amount of polypeptides of the invention in the tissue sample

1. A method of identifying a patient with a higher risk of preeclampsia comprising:
a) assaying a sample from said patient to determine the level of a mRNA or other polynucleotide within said sample that hybridizes specifically to a polynucleotide of SEQ ID NO: 1-852; and
b) comparing said level to a standard indicative of a higher risk of diagnosis of preeclampsia.

2. A method of identifying a patient with a higher risk of preeclampsia comprising:
a) contacting a sample from said patient with an antibody that specifically binds to a polypeptide of SEQ ID NO 853-1704 to determine the level of polypeptide within said sample; and
c) comparing said level to a standard indicative of a higher risk of diagnosis of preeclampsia.

3. The method of claim 1 or 2, wherein the specimen is serum.

4. The method of claim 1 or 2, wherein the specimen is plasma.

5. The method of claim 1 or 2, wherein the specimen is urine.

6. The method of claim 1 or 2, wherein the specimen is cervicovaginal mucous.

7. The method of claim 1 or 2, wherein the specimen is amniotic fluid.

8. The method of claim 1 or 2, wherein the specimen is fetal cells.

9. The method of claim 1, wherein said nucleotides further comprise any polymorphism or splice variants of the polynucleotide sequences of SEQ ID 1-852.

10. The method of claim 2, wherein said proteins further comprise any fragments of the polypeptide sequences of SEQ ID 853-1704.

11. A diagnostic kit for detecting preeclampsia comprising:
a) an antibody specific for any of the polypeptides of SEQ ID 853-1704 or fragments thereof; and
b) a standard for any of the polypeptides of SEQ ID 853-1704 indicative of a higher risk of diagnosis of preeclampsia.

12. A diagnostic kit for detecting preeclampsia comprising:
a) a polynucleotide sequence comprising any of SEQ ID 1-852 coupled to a surface; and
b) A standard for any of the polynucleotides of SEQ ID 1-852 indicative of a higher risk of diagnosis of preeclampsia.

13. A pharmaceutical composition comprising an antibody specific for any of the polypeptides of SEQ ID NO: 853-1704, effective to ameliorate signs or symptoms of preeclampsia.

14. The pharmaceutical composition of claim 13, wherein said antibody is a monoclonal antibody or fragment thereof.

15. A pharmaceutical composition comprising one or more purified polypeptides of SEQ ID NO: 853-1704, effective to ameliorate signs or symptoms of preeclampsia.

16. A method of treating preeclampsia comprising the steps of:
a) detecting increased levels of any of the polynucleotides of SEQ ID NO: 1-852 or the polypeptides of SEQ ID NO: 853-1704 in preeclamptic sample specimens; and
b) administering an effective dose of any of said compositions as in claims 13 or 14.

17. A method of treating preeclampsia comprising the steps of:
a) detecting decreased levels of any of the polynucleotides of SEQ ID NO: 1-852 or the polypeptides of SEQ ID NO: 853-1704 in preeclamptic sample specimens; and
b) administering an effective dose of any of said compositions as in claim 15.
